(19) United States
(12) Patent Application Publication
(10) Pub. No.: US 2022/0109108 A1
(43) Pub. Date: Apr. 7, 2022

(54) LIGHT EMITTING DEVICE AND POLYCYCLIC COMPOUND FOR THE SAME

(71) Applicant: Samsung Display Co., Ltd., Yongin-si (KR)

(72) Inventor: Takuya UNO, Yokohama (JP)

(73) Assignee: Samsung Display Co., Ltd., Yongin-si (KR)

(21) Appl. No.: 17/322,188

(22) Filed: May 17, 2021

(30) Foreign Application Priority Data
Sep. 22, 2020 (KR) 10-2020-0122259

(51) Int. Cl. H01L 51/00

(52) U.S. Cl. CPC H01L 51/0061; H01L 51/006; H01L 51/0054; H01L 51/0055; H01L 51/5056; H01L 51/0072; H01L 51/0073; H01L 51/0074; H01L 51/0056

(57) ABSTRACT

A light emitting device includes a first electrode, a second electrode disposed on the first electrode, and at least one functional layer disposed between the first electrode and the second electrode. The at least one functional layer includes a polycyclic compound represented by Formula 1, thereby providing a light emitting device having high luminous efficiency and improved life characteristics.

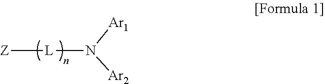

### CROSS-REFERENCE TO RELATED APPLICATION(S)

This application claims priority to and benefits of Korean Patent Application No. 10-2020-0122259 under 35 U.S.C. § 119, filed on Sep. 22, 2020 in the Korean Intellectual Property Office, the entire contents of which are incorporated herein by reference.

### BACKGROUND

### 1. Technical Field

The disclosure relates to a polycyclic compound used in a hole transport region and a light emitting device including the same.

### 2. Description of the Related Art

Active development continues for an organic electroluminescence display as an image display apparatus. The organic electroluminescence display includes a so-called self-luminescent light emitting device in which holes and electrons respectively injected from a first electrode and a second electrode recombine in an emission layer, and thus a luminescent material of the emission layer emits light to achieve display.

In the application of a light emitting device to a display apparatus, there is an ongoing demand for a light emitting device having low driving voltage, high luminous efficiency, and a long service life, and continuous development is required on materials for a light emitting device which is capable of stably attaining such characteristics.

In order to implement a light emitting device with high efficiency, development continues for materials of a hole transport region for suppressing the diffusion of exciton energy of the emission layer.

### SUMMARY

The disclosure provides a light emitting device exhibiting excellent luminous efficiency and long service life characteristics.

The disclosure also provides a polycyclic compound which is a material for a light emitting device having high efficiency and long service life characteristics.

An embodiment provides a polycyclic compound represented by Formula 1 below:

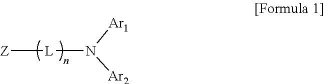

In Formula 1 above, n may e an integer from 0 to 3, L may be a substituted or unsubstituted arylene group having 6 to 40 ring-forming carbon atoms and excluding fluorene, or a substituted or unsubstituted heteroarylene group having 2 to 40 ring-forming carbon atoms and excluding N as a ring-forming atom, or may be bonded to Ar₁ or Ar₂ by using a single bond, O, S, or C(R₁)(R₂) as a linker to form a ring. Ar₁ and Ar₂ may each independently be a substituted or unsubstituted aryl group having 6 to 40 ring-forming carbon atoms and excluding triphenylene, or a substituted or unsubstituted heteroaryl group having 2 to 40 ring-forming carbon atoms, or may be bonded to L or an adjacent substituent by using a single bond, O, S, or C(R₁)(R₂) as a linker to form a ring, and R₁ and R₂ may each independently be a substituted or unsubstituted alkyl group having 1 to 20 carbon atoms, or a substituted or unsubstituted aryl group having 6 to 40 ring-forming carbon atoms, and Z may be a group represented by Formula 2-1 or Formula 2-2 below:

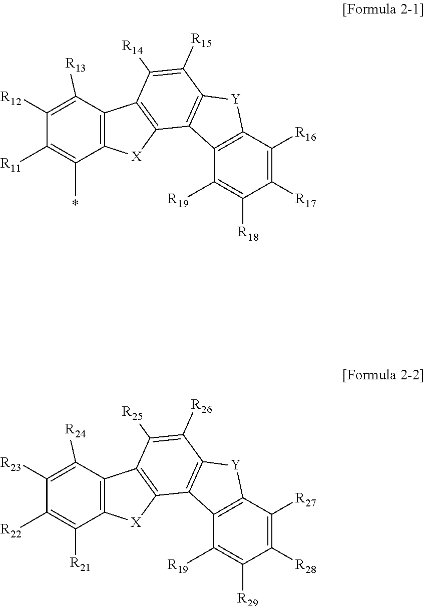

In Formula 2-1 and Formula 2-2 above, X and Y may each independently be O or S, and R₁₁ to R₁₉ and R₂₁ to R₂₉ may each independently be a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, or may be bonded to an adjacent group to form a ring, and * indicates a binding site to a neighboring atom.

In an embodiment, Formula 1 above may be represented by any one of Formula 1-1 to Formula 1-4 below.

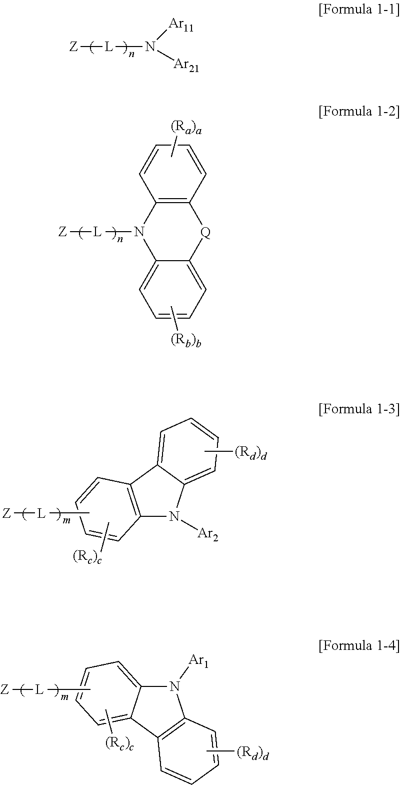

In Formula 1-1 above, Ar₁₁ and Ar₂₁ may each independently be a substituted or unsubstituted aryl group having 6 to 40 ring-forming carbon atoms and excluding triphenylene, or a substituted or unsubstituted heteroaryl group having 2 to 40 ring-forming carbon atoms and excluding N as a ring-forming atom. In Formula 1-2 above, Q may be a single bond, O, S, or C(R₁)(R₂), and a and b may each independently be an integer from 0 to 4, and in Formula 1-3 and Formula 1-4, m may be an integer from 0 to 2, c may be an integer from 0 to 3, d may be an integer from 0 to 4, and in Formula 1-2 to 1-4, Rₐ to R_{d} may each independently be a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms, or may be bonded to adjacent groups to form an aromatic ring. In Formula 1-1 to Formula 1-4 above, Z, L, n, R₁, R₂, Ar₁, and Ar₂ may be the same as defined in connection with Formula 1 above.

In an embodiment, Formula 2-1 above may be represented by any one among Formula 2-1A to Formula 2-1D below:

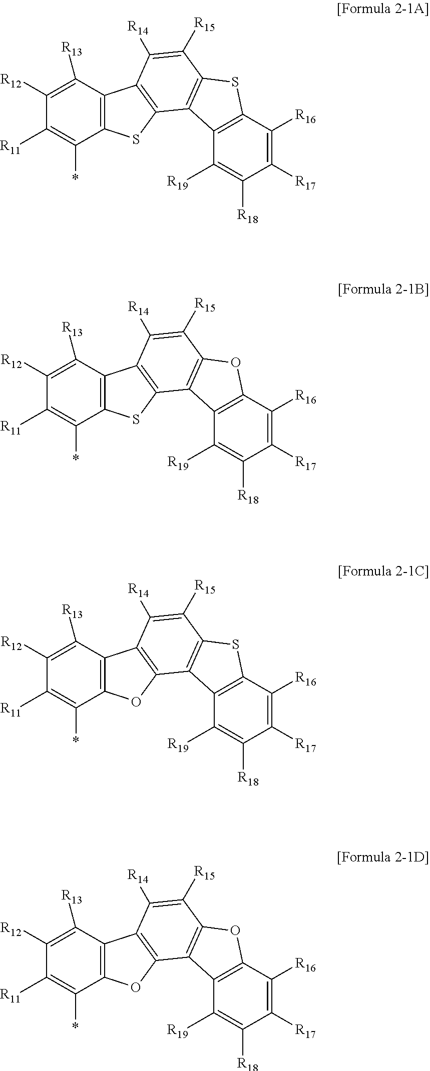

In Formula 2-1A to Formula 2-1D above, R₁₁ to R₁₉ and * may be the same as defined in connection with Formula 2-1 above.

In an embodiment, Formula 2-2 above may be represented by any one among Formula 2-2A to Formula 2-21D below:

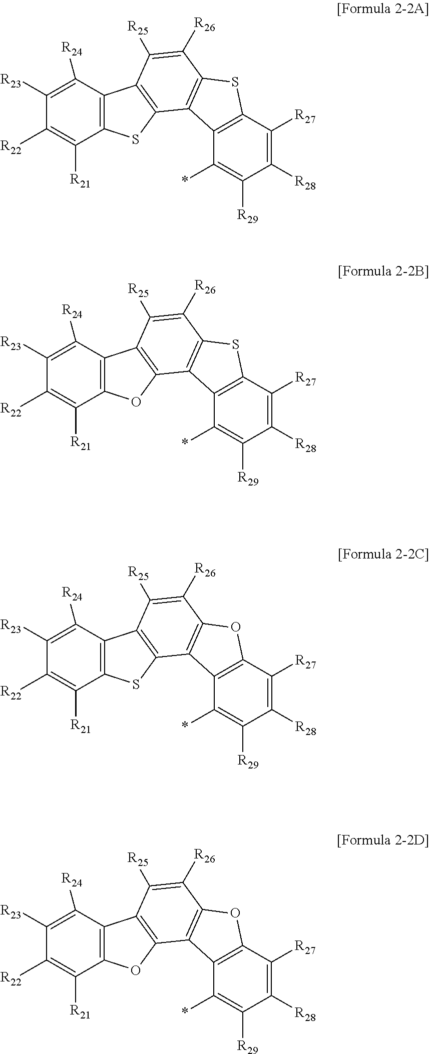

In Formula 2-2A to Formula 2-2D above, R₂₁ to R₂₉ and * may be the same as defined in connection with Formula 2-2 above.

In an embodiment, in Formula 1 above, L may be a direct linkage, an unsubstituted phenylene group, an unsubstituted divalent biphenyl group, an unsubstituted naphthalene group, an unsubstituted phenanthrene group, an unsubstituted dibenzofuranylene group, or an unsubstituted dibenzothiophenylene group.

In an embodiment, in Formula 2-1 above, two selected from among R₁₁ to R₁₃, R₁₄ and R₁₅, or two selected from among R₁₆ to R₁₉ may be bonded to each other to form a ring which is condensed with an adjacent benzene ring.

In an embodiment, in Formula 2-2 above, two selected from among R₂₁ to R₂₃, R₂₄ and R₂₅, or two selected from among R₂₆ to R₂₉ may be bonded to each other to form a ring which is condensed with an adjacent benzene ring.

In an embodiment, a polycyclic compound may be represented by Formula A below:

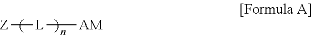

In Formula A above, n may be an integer from 0 to 3, L may be a substituted or unsubstituted arylene group having 6 to 40 ring-forming carbon atoms and excluding fluorene, or a substituted or unsubstituted heteroarylene group having 2 to 40 ring-forming carbon atoms and excluding N as a ring-forming atom, and AM may be a substituted or unsubstituted amine group, or a substituted or unsubstituted heterocyclic group including N as a ring-forming atom. Z may be a group represented by Formula 2-1 or Formula 2-2 below:

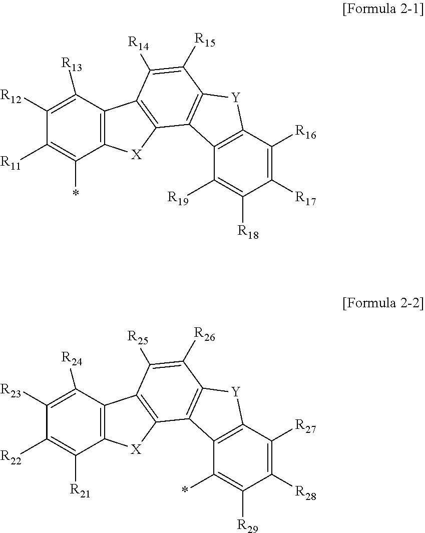

In Formula 2-1 and Formula 2-2 above, X and Y may each independently be O or S, R₁₁ to R₁₉ and R₂₁ to R₂₉ may each independently be a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, or may be bonded to an adjacent group to form a ring, and * indicates a binding site to a neighboring atom.

In an embodiment, the heterocyclic group may be a substituted or unsubstituted carbazole group, a substituted or unsubstituted phenoxazine group, a substituted or unsubstituted phenothiazine group, or substituted or unsubstituted acridine group.

In an embodiment, AM may be a group represented by any one among Formulae A1 to A3 below:

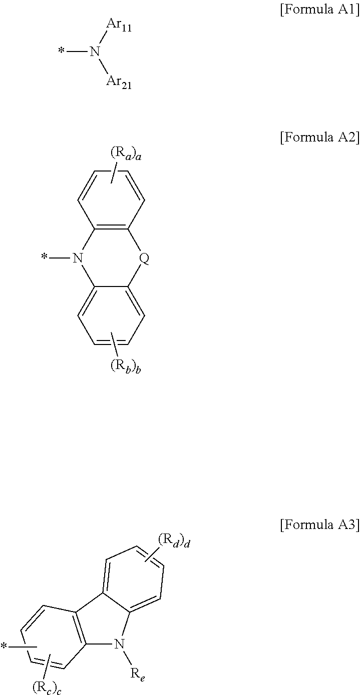

In Formula A1 above, Ar₁₁ and Ar₂₁ may each independently be a substituted or unsubstituted aryl group having 6 to 40 ring-forming carbon atoms and excluding triphenylene, or a substituted or unsubstituted heteroaryl group having 2 to 40 ring-forming carbon atoms and excluding N as a ring-forming atom. In Formula A2 above, Q may be a single bond, O, S, or C(R₁)(R₂), and R₁ and R₂ may each independently be a substituted or unsubstituted alkyl group having 1 to 20 carbon atoms, or a substituted or unsubstituted aryl group having 6 to 40 ring-forming carbon atoms. In Formula A2 and Formula A3 above, a, b, and d may each independently be an integer from 0 to 4, c may be an integer from 0 to 3, and Rₐ, R_{b}, R_{c}, R_{d}, and Rₑ may each independently be a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms, or may be bonded to an adjacent group to form an aromatic ring. In Formulae A1 to A3, * indicates a binding site to a neighboring atom.

In an embodiment, in Formula A above, L may be a direct linkage, a substituted or unsubstituted phenylene group, a substituted or unsubstituted divalent biphenyl group, a substituted or unsubstituted naphthalene group, a substituted or unsubstituted phenanthrene group, a substituted or unsubstituted dibenzofuranylene group, or a substituted or unsubstituted dibenzothiophenylene group.

In an embodiment, an organic electroluminescence device may include a first electrode, a second electrode disposed on the first electrode, and at least one functional layer disposed between the first electrode and the second electrode and including the above-described polycyclic compound of an embodiment.

In an embodiment, the at least one functional layer may include an emission layer, a hole transport region disposed between the first electrode and the emission layer, and an electron transport region disposed between the emission layer and the second electrode. The hole transport region may include the polycyclic compound.

In an embodiment, the hole transport region may include at least one of a hole injection layer, a hole transport layer, and an electron blocking layer, and at least one of the hole injection layer, the hole transport layer, and the electron blocking layer may include the polycyclic compound.

### BRIEF DESCRIPTION OF THE DRAWINGS

The accompanying drawings are included to provide a further understanding of the embodiment, and are incorporated in and constitute a part of this specification. The drawings illustrate embodiments of the disclosure, together with the description. The above and other aspects and features of the disclosure will become more apparent by describing in detail embodiments thereof with reference to the attached drawings, in which:

FIG. 1 is a plan view illustrating a display apparatus according to an embodiment;

FIG. 2 is a schematic cross-sectional view of a display apparatus according to an embodiment;

FIG. 3 is a schematic cross-sectional view illustrating a light emitting device according to an embodiment;

FIG. 4 is a schematic cross-sectional view illustrating a light emitting device according to an embodiment;

FIG. 5 is a schematic cross-sectional view illustrating a light emitting device according to an embodiment;

FIG. 6 is a schematic cross-sectional view illustrating a light emitting device according to an embodiment;

FIG. 7 is a schematic cross-sectional view illustrating a display apparatus according to an embodiment.

FIG. 8 is a schematic cross-sectional view illustrating a display apparatus according to an embodiment.

### DETAILED DESCRIPTION OF THE EMBODIMENTS

The embodiments may be modified in various forms, and thus embodiments will be represented in the drawings and described in detail. It should be understood, however, that it is not intended to limit the embodiments to the particular forms disclosed, but rather, is intended to cover all modifications, equivalents, and alternatives falling within the spirit and scope of the disclosure. The embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

The term “at least one of” is intended to include the meaning of “at least one selected from” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the invention. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within +20%, 10%, or 5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may mean substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. Each of the substituents described above may be substituted or unsubstituted. For example, a biphenyl group may be interpreted as an aryl group or a phenyl group substituted with a phenyl group.

In the specification, the phrase “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. The rings formed by being bonded to each other may be connected to another ring to form a spiro structure.

In the specification, the term “adjacent group” may mean a substituent substituted at an atom which is directly connected to an atom substituted with a corresponding substituent, another substituent substituted at an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. For example, two methyl groups in 4,5-dimethylphenanthrene may be interpreted as “adjacent groups” to each other.

In the specification, examples of a halogen atom may include a fluorine atom, a chlorine atom, a bromine atom, or an iodine atom.

In the specification, an alkyl group may be a linear, branched, or cyclic type. The number of carbon atoms in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments are not limited thereto.

In the specification, a hydrocarbon ring group may be any functional group or substituent derived from an aliphatic hydrocarbon ring. The hydrocarbon ring group may be a saturated hydrocarbon ring group having 5 to 20 ring-forming carbon atoms.

In the specification, an aryl group may be any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenylyl group, a terphenylyl group, a quaterphenylyl group, a quinquephenylyl group, a sexiphenylyl group, a triphenylenyl group, a pyrenyl group, a benzofluoranthenyl group, a chrysenyl group, etc., but embodiments are not limited thereto.

In the specification, a fluorenyl group may be substituted, and two substituents may be combined with each other to form a spiro structure. Examples of cases where the fluorenyl group is substituted are as follows. However, embodiments are not limited thereto.

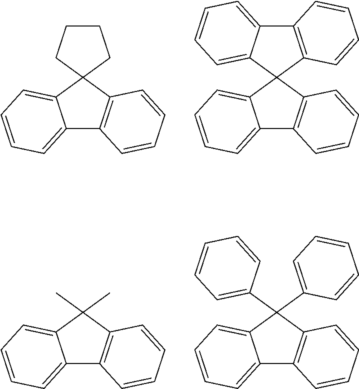

In the specification, a heterocyclic group may be any functional group or substituent derived from a ring including at least one of B, O, N, P, Si, and Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, a heterocyclic group may include at least one of B, O, N, P, Si, and S as a heteroatom. If the heterocyclic group includes two or more heteroatoms, the two or more heteroatoms may be the same or different. The heterocyclic group may be a monocyclic heterocyclic group or a polycyclic heterocyclic group and may be understood to include a heteroaryl group. The ring-forming carbon number of the heterocyclic group may be 2 to 30, 2 to 20, or 2 to 10.

In the specification, an aliphatic heterocyclic group may include one or more among B, O, N, P, Si, and S as a heteroatom. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments are not limited thereto.

In the specification, a heteroaryl group herein may include at least one of B, O, N, P, Si, and S as a heteroatom. When the heteroaryl group contains two or more heteroatoms, the two or more heteroatoms may be the same as or different from each other. The heteroaryl group may be a monocyclic heteroaryl group or polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a triazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazolyl group, a thiadiazole group, a phenothiazine group, a dibenzosilole group, a dibenzofuran group, etc., but embodiments are not limited thereto.

In the specification, the above description with respect to the aryl group may be applied to an arylene group except that the arylene group is a divalent group. The explanation on the aforementioned heteroaryl group may be applied to the heteroarylene group except that the heteroarylene group is a divalent group.

In the specification, a silyl group may include an alkylsilyl group and an arylsilyl group. Examples of the silyl group may include trimethylsilyl, triethylsilyl, t-butyldimethylsilyl, vinyldimethylsilyl, propyldimethylsilyl, triphenylsilyl, diphenylsilyl, phenylsilyl, etc. However, embodiments are not limited thereto.

In the specification, the number of carbon atoms in an amino group is not specifically limited, but may be 1 to 30. The amino group may include an alkyl amino group, an aryl amino group, or a heteroaryl amino group. Examples of the amino group include a methylamino group, a dimethylamino group, a phenylamino group, a diphenylamino group, a naphthylamino group, a 9-methyl-anthracenylamino group, a triphenylamino group, etc., but are not limited thereto.

In the specification, the number of ring-forming carbon atoms in a carbonyl group may be 1 to 40, 1 to 30, or 1 to 20. For example, the carbonyl group may have the following structures, but embodiments are not limited thereto.

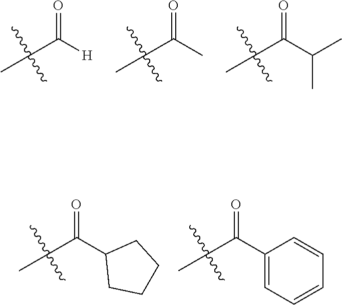

In the specification, the number of carbon atoms in a sulfinyl group and a sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, a thio group may include an alkylthio group and an arylthio group. The thio group may mean that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments are limited thereto.

In the specification, an oxy group may include an oxygen atom that is bonded to the alkyl group or the aryl group as defined above. The oxy group may include an alkoxy group and an aryl oxy group. The alkoxy group may be a linear chain, a branched chain, or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., without limitation.

In the specification, a boron group may include a boron atom that is bonded to the alkyl group or the aryl group as defined above. The boron group includes an alkyl boron group and an aryl boron group. Examples of the boron group may include a trimethylboron group, a triethylboron group, a t-butyldimethylboron group, a triphenylboron group, a diphenylboron group, a phenylboron group, etc., but embodiments are not limited thereto.

In the specification, an alkenyl group may be linear or branched. The number of carbon atoms in the alkenyl group is not specifically limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, etc., but embodiments are not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, etc., but embodiments are not limited thereto.

In the specification, the alkyl group among an alkylthio group, an alkylsulfoxy group, an alkylaryl group, an alkylamino group, an alkyl boron group, an alkyl silyl group, and an alkyl amine group may be the same as the examples of the alkyl group described above.

In the specification, the aryl group among an aryloxy group, an arylthio group, an arylsulfoxy group, an arylamino group, an arylboron group, an arylsilyl group, an arylamine group may be the same as the examples of the aryl group described above.

In the specification, a direct linkage may be a single bond.

In the specification,

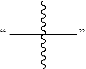

and

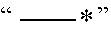 [SMILES: *C]

each indicate a binding site to a neighboring atom.

Hereinafter, embodiments will be described with reference to the accompanying drawings.

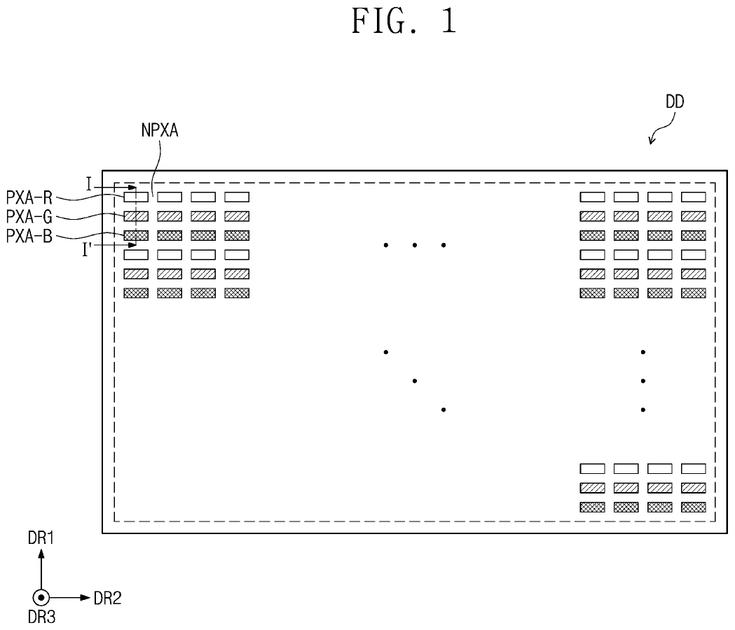

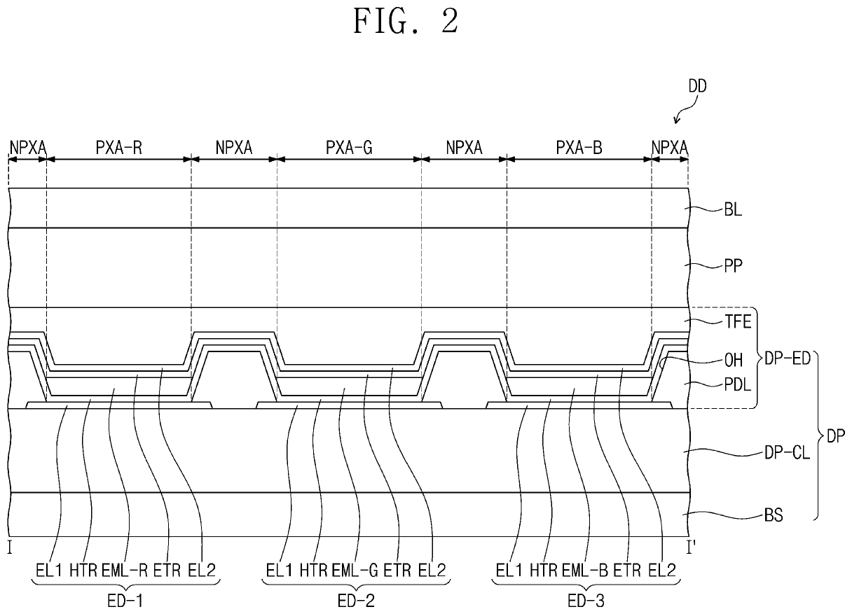

FIG. 1 is a plan view illustrating an embodiment of a display apparatus DD. FIG. 2 is a schematic cross-sectional view of the display apparatus DD of the embodiment. FIG. 2 is a schematic cross-sectional view illustrating a part taken along line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include multiple light emitting devices ED-1, ED-2, and ED-3. The optical layer PP may be disposed on the display panel DP and control light reflected from an external light at the display panel DP. The optical layer PP may include, for example, a polarization layer or a color filter layer. While not shown in the drawing, the optical layer PP may be omitted from the display apparatus DD in another embodiment.

A base substrate BL may be disposed on the optical layer PP. The base substrate BL may be a member which provides a base surface on which the optical layer PP disposed. The base substrate BL may be a glass substrate, a metal substrate, a plastic substrate, etc. However, embodiments are not limited thereto, and the base substrate BL may be an inorganic layer, an organic layer, or a composite material layer. While not shown in the drawings, in an embodiment, the base substrate BL may be omitted.

The display apparatus DD according to an embodiment may further include a filling layer (not shown). The filling layer (not shown) may be disposed between a display device layer DP-ED and the base substrate BL. The filling layer (not shown) may be an organic material layer. The filling layer (not shown) may include at least one of an acrylic-based resin, a silicone-based resin, and an epoxy-based resin.

The display panel DP may include a base layer BS, a circuit layer DP-CL provided on the base layer BS, and a display device layer DP-ED. The display device layer DP-ED may include a pixel defining layer PDL, the light emitting devices ED-1, ED-2, and ED-3 disposed between portions of the pixel defining layer PDL, and an encapsulation layer TFE disposed on the light emitting devices ED-1, ED-2, and ED-3.

The base layer BS may be a member which provides a base surface on which the display device layer DP-ED is disposed. The base layer BS may be a glass substrate, a metal substrate, a plastic substrate, etc. However, embodiments are not limited thereto, and the base layer BS may be an inorganic layer, an organic layer, or a composite material layer.

In an embodiment, the circuit layer DP-CL is disposed on the base layer BS, and the circuit layer DP-CL may include transistors (not shown). Each of the transistors (not shown) may include a control electrode, an input electrode, and an output electrode. For example, the circuit layer DP-CL may include a switching transistor and a driving transistor in order to drive the light emitting devices ED-1, ED-2, and ED-3 of the display device layer DP-ED.

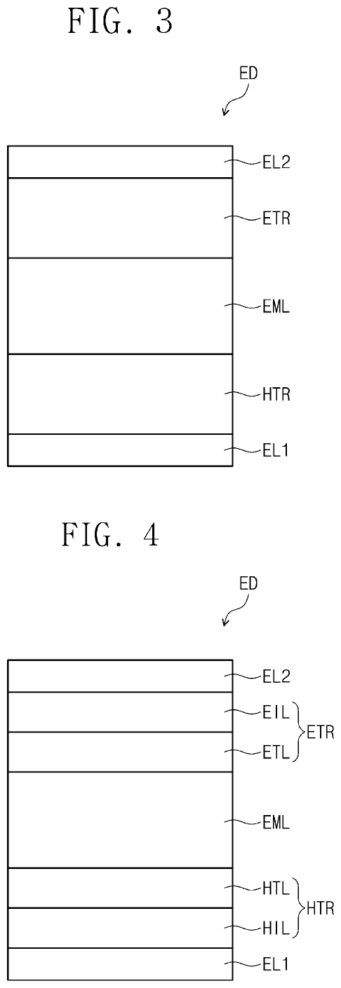

Each of the light emitting devices ED-1, ED-2, and ED-3 may have a structure of a light emitting device ED of an embodiment according to FIGS. 3 to 6, which will be described later. For example, the light emitting devices ED-1, ED-2, and ED-3 may each include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting devices ED-1, ED-2, and ED-3 in the openings OH defined in the pixel defining layer PDL, and the hole transport region HTR, the electron transport region ETR, and the second electrode EL2 are provided as common layers in the light emitting devices ED-1, ED-2, and ED-3. However, embodiments are not limited thereto, and unlike the feature illustrated in FIG. 2, the hole transport region HTR and the electron transport region ETR in an embodiment may be provided by being patterned inside the opening OH defined in the pixel defining film PDL. For example, the hole transport region HTR, the emission layers EML-R, EML-G, and EML-B, and the electron transport region ETR in an embodiment may be provided by being patterned using an inkjet printing method.

The encapsulation layer TFE may cover the light emitting devices ED-1, ED-2, and ED-3. The encapsulation layer TFE may seal the display device layer DP-ED. The encapsulation layer TFE may be a thin film encapsulation layer. The encapsulation layer TFE may be formed by laminating one layer or multiple layers. The encapsulation layer TFE may include at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film may protect the display device layer DP-ED from moisture and/or oxygen, and the encapsulation-organic film may protect the display device layer DP-ED from foreign substances such as dust particles. The encapsulation-inorganic film may include silicon nitride, silicon oxynitride, silicon oxide, titanium oxide, aluminum oxide, or the like, but embodiments are not limited thereto. The encapsulation-organic film may include an acrylic-based compound, an epoxy-based compound, or the like. The encapsulation-organic film may include a photopolymerizable organic material, but embodiments not limited thereto.

The encapsulation layer TFE may be disposed on the second electrode EL2 and may be disposed to fill the opening OH.

Referring to FIGS. 1 and 2, the display apparatus DD may include a non-light emitting region NPXA and light emitting regions PXA-R, PXA-G and PXA-B. The light emitting regions PXA-R, PXA-G, and PXA-B may each be a region which emits light generated from the light emitting devices ED-1, ED-2, and ED-3, respectively. The light emitting regions PXA-R, PXA-G, and PXA-B may be spaced apart from each other in a plane.

Each of the light emitting regions PXA-R, PXA-G, and PXA-B may be a region divided by pixel defining layer PDL. The non-light emitting regions NPXA may be regions between the adjacent light emitting regions PXA-R, PXA-G, and PXA-B, which correspond to portions of the pixel defining layer PDL. In the specification, each of the light emitting regions PXA-R, PXA-G, and PXA-B may correspond to a pixel. The pixel defining layer PDL may separate the light emitting devices ED-1, ED-2, and ED-3. The emission layers EML-R, EML-G, and EML-B of the light emitting devices ED-1, ED-2, and ED-3 may be disposed in openings OH defined by the pixel defining layer PDL and separated from each other.

The light emitting regions PXA-R, PXA-G, and PXA-B may be divided into groups according to the color of light generated from the light emitting devices ED-1, ED-2, and ED-3. In the display apparatus DD of an embodiment shown in FIGS. 1 and 2, three light emitting regions PXA-R, PXA-G, and PXA-B which emit red light, green light, and blue light, respectively are illustrated. For example, the display apparatus DD of an embodiment may include the red light emitting region PXA-R, the green light emitting region PXA-G, and the blue light emitting region PXA-B which are different.

In the display apparatus DD according to an embodiment, the light emitting devices ED-1, ED-2, and ED-3 may emit light in different wavelength regions. For example, in an embodiment, the display apparatus DD may include a first light emitting device ED-1 that emits red light, a second light emitting device ED-2 that emits green light, and a third light emitting device ED-3 that emits blue light. For example, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, embodiments are not limited thereto, and the first to the third light emitting devices ED-1, ED-2, and ED-3 may emit light in the same wavelength range or at least one light emitting device may emit light in a wavelength range different from the others. For example, the first to third light emitting devices ED-1, ED-2, and ED-3 may all emit blue light.

The light emitting regions PXA-R, PXA-G, and PXA-B in the display apparatus DD according to an embodiment may be arranged in a stripe form. Referring to FIG. 1, the red light emitting regions PXA-R, the green light emitting regions PXA-G, and the blue light emitting regions PXA-B each may be arranged along a second directional axis DR2. The red light emitting region PXA-R, the green light emitting region PXA-G, and the blue light emitting region PXA-B may be alternately arranged in this order along a first directional axis DR1.

FIGS. 1 and 2 illustrate that the light emitting regions PXA-R, PXA-G, and PXA-B have a similar area, but embodiments are not limited thereto, and the light emitting regions PXA-R, PXA-G, and PXA-B may have different areas from each other according to a wavelength range of the emitted light. For example, the areas of the light emitting regions PXA-R, PXA-G, and PXA-B may be areas in a plan view that are defined by the first directional axis DR1 and the second directional axis DR2.

The arrangement form the light emitting regions PXA-R, PXA-G, and PXA-B is not limited to what is illustrated in FIG. 1, and the order in which the red light emitting region PXA-R, the green light emitting region PXA-G, and the blue light emitting region PXA-B are arranged may be variously combined and provided according to characteristics of a display quality required in the display apparatus DD. For example, the arrangement of the light emitting regions PXA-R, PXA-G, and PXA-B may be a PenTile® arrangement or a diamond arrangement.

The areas of the light emitting regions PXA-R, PXA-G, and PXA-B may be different from each other. For example, in an embodiment, the area of the green light emitting region PXA-G may be smaller than that of the blue light emitting region PXA-B, but embodiments are not limited thereto.

Hereinafter, FIGS. 3 to 6 are schematic cross-sectional views illustrating light emitting devices according to an embodiment. The light emitting devices ED according to embodiments may each include a first electrode EL1, a second electrode EL2 facing the first electrode EL1, and at least one functional layer disposed between the first electrode EL1 and the second electrode EL2. The at least one functional layer may include a hole transport region HTR, an emission layer EML, and an electron transport region ETR that are sequentially stacked. For example, the light emitting devices ED of embodiments may each include the first electrode EL1, the hole transport region HTR, the emission layer EML, the electron transport region ETR, and the second electrode EL2, stacked in that order.

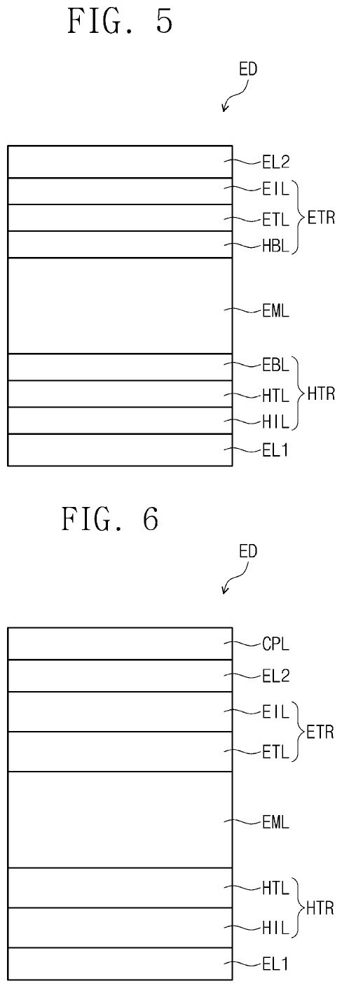

Compared to FIG. 3, FIG. 4 illustrates a schematic cross-sectional view of a light emitting device ED of an embodiment, in which a hole transport region HTR includes a hole injection layer HIL and a hole transport layer HTL, and an electron transport region ETR includes an electron injection layer EIL and an electron transport layer ETL. In comparison to FIG. 3, FIG. 5 illustrates a schematic cross-sectional view of a light emitting device ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared to FIG. 4, FIG. 6 illustrates a schematic cross-sectional view of a light emitting device ED of an embodiment including a capping layer CPL disposed on the second electrode EL2.

The light emitting device ED of an embodiment may include the polycyclic compound of an embodiment, which will be described below, in at least one functional layer of the hole transport region HTR, the emission layer EML, the electron transport region ETR, or the like.

In the light emitting device ED according to an embodiment, the first electrode EL1 has conductivity. The first electrode EL1 may be formed of a metal material, a metal alloy, or a conductive compound. The first electrode EL1 may be an anode or a cathode. However, embodiments are not limited thereto. The first electrode EL1 may be a pixel electrode. The first electrode EL1 may be a transmissive electrode, a transflective electrode, or a reflective electrode. If the first electrode EL1 is a transmissive electrode, the first electrode EL1 may be formed using a transparent metal oxide such as indium tin oxide (ITO), indium zinc oxide (IZO), zinc oxide (ZnO), and indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, W, a compound thereof, or a mixture thereof (e.g., a mixture of Ag and Mg). In other embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but embodiments are not limited thereto. As embodiments are not limited thereto, the first electrode EL1 may include the above-described metal materials, combinations of at least two metal materials of the above-described metal materials, oxides of the above-described metal materials, or the like. A thickness of the first electrode EL1 may be in a range of about 700 Å to about 10,000 Å. For example, the thickness of the first electrode EL1 may be in a range of about 1,000 Å to about 3,000 Å.

The hole transport region HTR is provided on the first electrode EL1. The hole transport region HTR may include at least one of a hole injection layer HIL, a hole transport layer HTL, a buffer layer (not shown), an emission-auxiliary layer (not shown), and an electron blocking layer EBL. A thickness of the hole transport region HTR may be, for example, in a range of about 50 Å to about 15,000 Å.

The hole transport region HTR may have a single layer formed of a single material, a single layer formed of different materials, or a multilayer structure including multiple layers formed of different materials.

For example, the hole transport region HTR may have a single layer structure of the hole injection layer HIL or the hole transport layer HTL, and may have a single layer structure formed of a hole injection material and a hole transport material. The hole transport region HTR may have a single layer structure formed of different materials, or a structure in which a hole injection layer HIL/hole transport layer HTL, a hole injection layer HIL/hole transport layer HTL/buffer layer (not shown), a hole injection layer HIL/buffer layer (not shown), a hole transport layer HTL/buffer layer (not shown), or a hole injection layer HIL/hole transport layer HTL/electron blocking layer EBL are stacked in order from the first electrode EL1, but embodiments are not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR in the light emitting device ED of an embodiment may include a polycyclic compound represented by Formula 1 below. The hole transport region HTR in the light emitting device ED of an embodiment may include at least one of the hole injection layer HIL, the hole transport layer HTL, and the electron blocking layer EBL, and at least one of the hole injection layer HIL, the hole transport layer HTL, and the electron blocking layer EBL may include the polycyclic compound represented by Formula 1. For example, the hole transport layer HTL in the light emitting device ED of an embodiment may include a polycyclic compound represented by Formula 1 below:

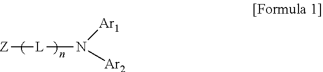

In Formula 1, Z may correspond to a benzobisdibenzoheterol moiety. For example, the polycyclic compound represented by Formula 1 of an embodiment may have a molecular structure in which a polycyclic heterocycle of the benzobisdibenzoheterol moiety

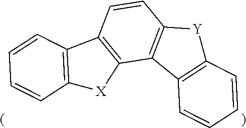

and an amine derivative

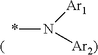

are bonded.

In Formula 1, n may be an integer from 0 to 3. In Formula 1, L may be a substituted or unsubstituted arylene group having 6 to 40 ring-forming carbon atoms and excluding fluorene, or a substituted or unsubstituted heteroarylene group having 2 to 40 ring-forming carbon atoms and excluding N as a ring-forming atom, or may be bonded to Ar₁ or Ar₂ by using a single bond, O, S, or C(R₁)(R₂) as a linker to form a ring. For example, at least one of L(s) may be bonded to Ar₁ or Ar₂ to form a ring, or may be bonded to a substituent of Ar₁ or Ar₂ to form a ring.

In the group C(R₁)(R₂), R₁ and R₂ may each independently be a substituted or unsubstituted alkyl group having 1 to 20 carbon atoms, or a substituted or unsubstituted aryl group having 6 to 40 ring-forming carbon atoms. For example, R₁ and R₂ may each be a methyl group. However, embodiments are not limited thereto.

In Formula 1, when n is 0, Z and N (nitrogen atom) of the amine derivative may be directly linked. For example, when n is 0, Z and N of the amine derivative may be linked to each other by a single bond.

When n is an integer of 2 or more, multiple L(s) may all be the same, or at least one of the L(s) may be different from the others.

In an embodiment, in Formula 1, L may be a direct linkage, a divalent aryl group, or a divalent heteroaryl group. For example, L may be a direct linkage, a substituted or unsubstituted phenylene group, a substituted or unsubstituted divalent biphenyl group, a substituted or unsubstituted naphthalene group, a substituted or unsubstituted phenanthrene group, a substituted or unsubstituted dibenzofuranylene group, or a substituted or unsubstituted dibenzothiophenylene group. For example, L may be a direct linkage, an unsubstituted phenylene group, an unsubstituted divalent biphenyl group, an unsubstituted naphthalene group, an unsubstituted phenanthrene group, an unsubstituted dibenzofuranylene group, or an unsubstituted dibenzothiophenylene group. However, embodiments are not limited thereto.

In Formula 1, Ar₁ and Ar₂ may each independently be a substituted or unsubstituted aryl group having 6 to 40 ring-forming carbon atoms and excluding triphenylene, or a substituted or unsubstituted heteroaryl group having 2 to 40 ring-forming carbon atoms, or may be bonded to an adjacent group, by using a single bond, O, S, or C(R₁)(R₂) as a linker, to form a ring.

When Ar₁ and Ar₂ are bonded to an adjacent group to form a ring, by using a single bond, O, S, or C(R₁)(R₂) as a linker, Ar₁ and Ar₂ may be bonded to L or to an adjacent substituent to form a ring. For example, by using a single bond, O, S, or C(R₁)(R₂) as a linker, Ar₁, Ar₂, and a nitrogen atom of the amine derivative may be bonded to form a ring, any one of Ar₁ and Ar₂ and a nitrogen atom of the amine derivative may be bonded to form a ring, or any one of Ar₁ and Ar₂, a nitrogen atom of the amine derivative, and L may be bonded to form a ring.

In the group C(R₁)(R₂), R₁ and R₂ may each independently be a substituted or unsubstituted alkyl group having 1 to 20 carbon atoms, or a substituted or unsubstituted aryl group having 6 to 40 ring-forming carbon atoms. For example, R₁ and R₂ may each be a methyl group. However, embodiments are not limited thereto.

In an embodiment, Ar₁ and Ar₂ may each independently be an aryl group such as a substituted or unsubstituted phenyl group, a substituted or unsubstituted naphthyl group, a substituted or unsubstituted biphenyl group, a substituted or unsubstituted phenanthrene group, a substituted or unsubstituted terphenyl group, or a substituted or unsubstituted fluorene group. The substituted or unsubstituted fluorene group may be a fluorene group substituted with an aryl group, or may be that two substituents substituted are bonded to form a spiro structure. In an embodiment, Ar₁ and Ar₂ may each independently be a heteroaryl group such as a substituted or unsubstituted dibenzofuran group, or a substituted or unsubstituted dibenzothiophene group. However, embodiments are not limited thereto.

In an embodiment, when Ar₁ and Ar₂ may each independently be a substituted or unsubstituted aryl group having 6 to 40 ring-forming carbon atoms and excluding triphenylene, or a substituted or unsubstituted heteroaryl group having 2 to 40 ring-forming carbon atoms, the case where a substituent is an amino group, a nitro group, or a carbazole group is excluded.

In an embodiment, Ar₁ and Ar₂ may each independently be substituted with a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group. In Formula 1, a substituent substituted at Ar₁ or Ar₂ may be bonded to an adjacent group to form a ring. For example, a substituent substituted at Ar₁ or Ar₂ may be bonded to Ar₁ or Ar₂ to form a ring, or may be bonded to an adjacent another substituent and Ar₁ or Ar₂ to form a ring. However, embodiments are not limited thereto.

In the polycyclic compound represented by Formula 1 of an embodiment, two selected from among L, Ar₁, and Ar₂ may be bonded to each other to form a ring. For example, L and Ar₁ may be bonded to each other to form a ring, L and Ar₂ may be bonded to each other to form a ring, or Ar₁ and Ar₂ may be bonded to each other to form a ring. In Formula 1, Z may be a group represented by Formula 2-1 or Formula 2-2 below. In Formula 2-1 and Formula 2-2, * indicates a binding site to a neighboring atom, such as to a nitrogen atom of the amine derivative or to L.

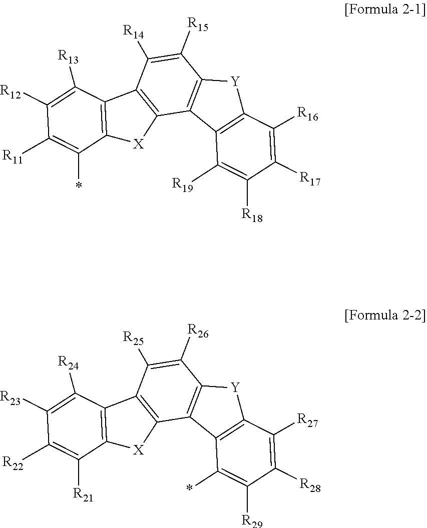

In Formula 2-1 and Formula 2-2, X and Y may each independently be O or S. For example, both X and Y may be S, both X and Y may be O, or one of X or Y may be O and the other may be S.

In Formula 2-1 and Formula 2-2, R₁₁ to R₁₉ and R₂₁ to R₂₉ may each independently be a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, or may be bonded to an adjacent group to form a ring.

For example, in Formula 2-1, R₁₁ to R₁₉ may all be hydrogen atoms. However, embodiments are not limited thereto, and at least one among R₁₁ to R₁₉ may be a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, and the others may be hydrogen atoms.

In Formula 2-1 and Formula 2-2, the case where R₁₁ to R₁₉ and R₂₁ to R₂₉ are amino groups or nitro groups is excluded. For example, in a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms, the case where a substituent is an amino group, a nitro group, or a carbazole group may be excluded.

In Formula 2-1, adjacent substituents among R₁₁ to R₁₉ may be bonded to each other to form a ring. For example, two selected from among R₁₁ to R₁₃, R₁₄ and R₁₅, or two selected from among R₁₆ to R₁₉ are bonded to each other to form a ring which is condensed with an adjacent benzene ring. Two selected from among R₁₁ to R₁₃, R₁₄ and R₁₅, or two selected from among R₁₆ to R₁₉ may be bonded to each other to form a benzene ring which may be bonded to a benzene ring of a benzobisdibenzoheterol skeleton to form a condensed ring.

For example, in Formula 2-2, R₂₁ to R₂₉ may all be hydrogen atoms. However, embodiments are not limited thereto, and at least one among R₂₁ to R₂₉ may be a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, and the others may be hydrogen atoms.

In Formula 2-2, adjacent substituents among R₂₁ to R₂₉ may be bonded to each other to form a ring. For example, two selected from among R₂₁ to R₂₃, R₂₄ and R₂₅, or two selected from among R₂₆ to R₂₉ are bonded to each other to form a ring which is condensed with an adjacent benzene ring. Two selected from among R₂₁ to R₂₃, R₂₄ and R₂₅, or two selected from among R₂₆ to R₂₉ may be bonded to each other to form a benzene ring which may be bonded to a benzene ring of a benzobisdibenzoheterol skeleton to form a condensed ring.

Formula 2-1 may be represented by any one among Formula 2-1A to Formula 2-1D below:

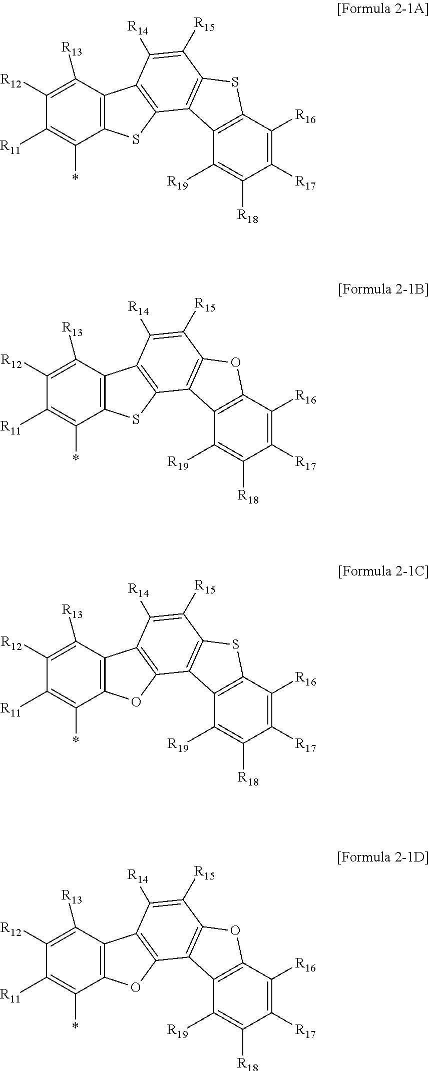

In Formula 2-1A to Formula 2-1D, R₁₁ to R₁₉ and * may be the same as defined in connection with Formula 2-1.

Formula 2-2 may be represented by any one of Formula 2-2A to Formula 2-2D below:

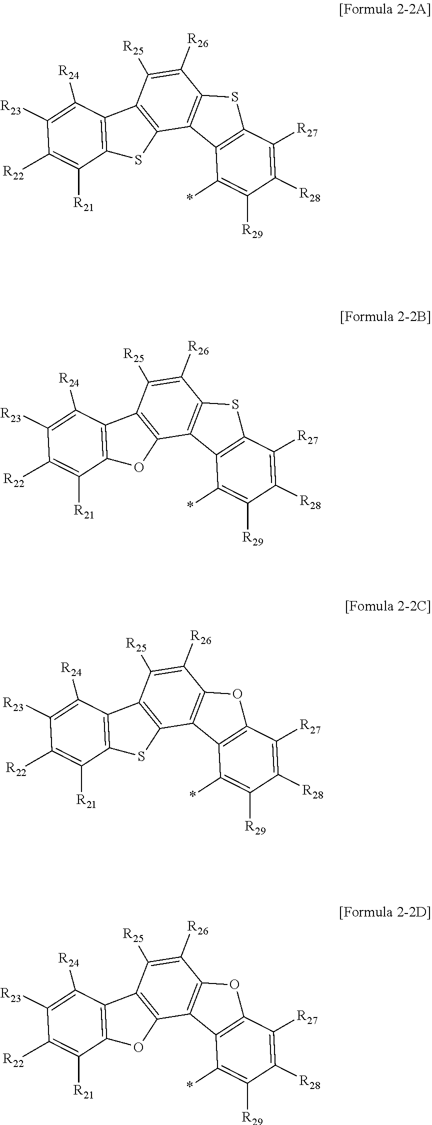

In Formula 2-2A to Formula 2-2D, R₂₁ to R₂₉ and * may be the same as defined in connection with Formula 2-2.

In an embodiment, Formula 1 may be represented by any one among Formula 1-1 to Formula 1-4 below:

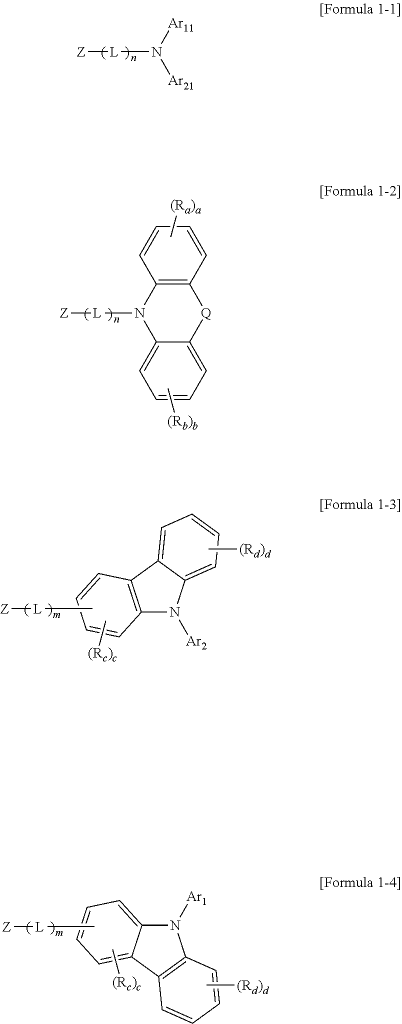

Formula 1-2 represents a case where Ar₁ and Ar₂ in Formula 1 are bonded to each other via Q as a linker to form a heterocycle along with N of the amine derivative. Formula 1-3 represents a case where Ar₁ in Formula 1, N of the amine derivative, and linker L are bonded to one another to form a ring, and Formula 1-4 represents a case where Ar₂ in Formula 1, N of the amine derivative, and linker L are bonded to one another to form a ring.

In Formula 1-1, Ar₁₁ and Ar₂₁ may each independently be a substituted or unsubstituted aryl group having 6 to 40 ring-forming carbon atoms and excluding triphenylene, or a substituted or unsubstituted heteroaryl group having 2 to 40 ring-forming carbon atoms and excluding N as a ring-forming atom.

In Formula 1-2, Q may be a single bond, O, S, or C(R₁)(R₂), and R₁ and R₂ may each independently be a substituted or unsubstituted alkyl group having 1 to 20 carbon atoms, or a substituted or unsubstituted aryl group having 6 to 40 ring-forming carbon atoms.

In Formula 1-2, a and b may each independently be an integer from 0 to 4, and Rₐ and R_{b} may each independently be a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, a substituted or unsubstituted heteroaryl group having 2 to 40 ring-forming carbon atoms, or bonded to an adjacent group to form an aromatic ring. When a is an integer of 2 or greater, multiple Rₐ(s) may all be the same or at least one may be different from the others. When b is an integer of 2 or more, multiple R_{b}(s) may all be the same or at least one may be different from the others.

In Formula 1-3 and Formula 1-4, m may be defined as an integer from 0 to (n−1). For example, in Formula 1-3 and Formula 1-4, m may be an integer from 0 to 2. When m is an integer of 2 or greater, multiple L(s) may all be the same or at least one may be different from the others.

In Formula 1-3 and Formula 1-4, c may be an integer from 0 to 3, and d may be an integer from 0 to 4. In Formula 1-3 and Formula 1-4, R_{c} and R_{d} may each independently be a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms. When c is an integer of 2 or greater, multiple R_{c}(s) may all be the same or at least one may be different from the others. When d is an integer of 2 or greater, multiple R_{d}(s) may all be the same or at least one may be different from the others. Further, Rₐ to R_{d} may each independently be bonded to an adjacent group to form an aromatic ring. For example, Rₐ to R_{d} may be bonded to adjacent groups to form an aromatic ring which is condensed to a substituted aromatic ring.

In Formula 1-1 to Formula 1-4, Z, L, n, R₁, R₂, Ar₁, and Ar₂ may be the same as defined in connection with Formula 1.

The polycyclic compound of an embodiment may be represented by Formula A below. In the description of the polycyclic compound represented by Formula A of an embodiment, the same as those described in Formula 1 may be applied with respect to the same symbols (or letters) as those indicated in the above-described polycyclic compound represented by Formula 1.

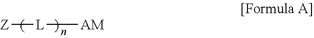

In Formula A, n may be an integer from 0 to 3, and L may be a substituted or unsubstituted arylene group having 6 to 40 ring-forming carbon atoms and excluding fluorene, or a substituted or unsubstituted heteroarylene group having 2 to 40 ring-forming carbon atoms and excluding N as a ring-forming atom. As described in the above-described polycyclic compound represented by Formula 1, in the polycyclic compound represented by Formula A of an embodiment, L may be a direct linkage, an unsubstituted phenylene group, an unsubstituted divalent biphenyl group, an unsubstituted naphthalene group, an unsubstituted phenanthrene group, an unsubstituted dibenzofuranylene group, or an unsubstituted dibenzothiophenylene group. However, embodiments are not limited thereto.

In Formula A, AM may be a substituted or unsubstituted amine group, or a substituted or unsubstituted heterocyclic group including N as a ring-forming atom. For example, AM in Formula A may be an amine derivative.

When AM is a substituted or unsubstituted amine group, an aryl group having 6 to 30 carbon atoms, a heteroaryl group having 2 to 30 carbon atoms, or the like may be included as a substituent. For example, AM may be an aryl amine group, but embodiments are not limited thereto.

When AM is a substituted or unsubstituted heterocyclic group, the heterocyclic group may be a substituted or unsubstituted carbazole group, a substituted or unsubstituted phenoxazine group, a substituted or unsubstituted phenothiazine group, or a substituted or unsubstituted acridine group.

In an embodiment, AM may be a group represented by any one among Formulae A1 to A3 below:

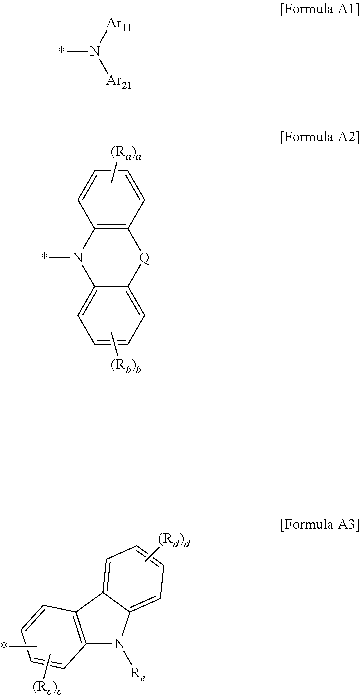

In Formula A1, Ar₁₁ and Ar₂₁ may each independently be a substituted or unsubstituted aryl group having 6 to 40 ring-forming carbon atoms and excluding triphenylene, or a substituted or unsubstituted heteroaryl group having 2 to 40 ring-forming carbon atoms and excluding N as a ring-forming atom.

In Formula A2, Q may be a single bond, O, S, or C(R₁)(R₂), and R₁ and R₂ may each independently be a substituted or unsubstituted alkyl group having 1 to 20 carbon atoms, or a substituted or unsubstituted aryl group having 6 to 40 ring-forming carbon atoms.

In Formula A2 and Formula A3 above, a, b, and d may each independently be an integer from 0 to 4, c may be an integer from 0 to 3, and Rₐ, R_{b}, R_{c}, R_{d}, and Rₑ may each independently be a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms, or may be bonded to an adjacent group to form an aromatic ring.

When a is an integer of 2 or greater, multiple Rₐ(s) may all be the same or at least one may be different from the others. When b to d each are an integer of 2 or greater, R_{b} to R_{d} may be equally explained.

In the polycyclic compound represented by Formula A of an embodiment, Z may be a benzobisdibenzoheterol moiety represented by Formula 2-1 or Formula 2-2 as described above in Formula 1.

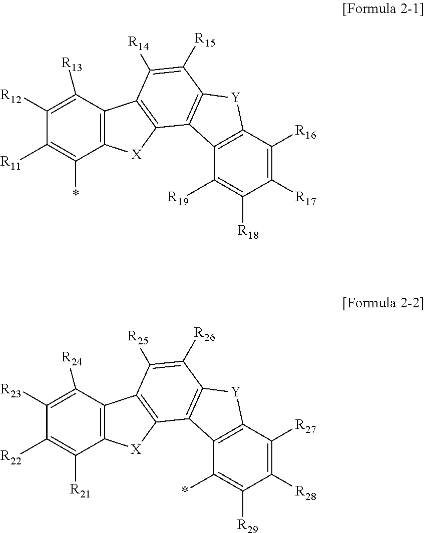

The same as those described in the polycyclic compound represented by Formula 1 as described above may be applied with respect to Formula 2-1 and Formula 2-2. In Formula 2-1 and Formula 2-2, * indicates a binding site to a neighboring atom. For example, in Formula 2-1 and Formula 2-2, * may indicate a binding site to AM or to L.

The polycyclic compound represented by Formula 1 or Formula A of an embodiment may be one selected from Compound Group 1A to Compound Group 1H below. The hole transport region HTR of the light emitting device ED of an embodiment may include at least one among the polycyclic compounds disclosed in Compound Group 1A to Compound Group 1H below:

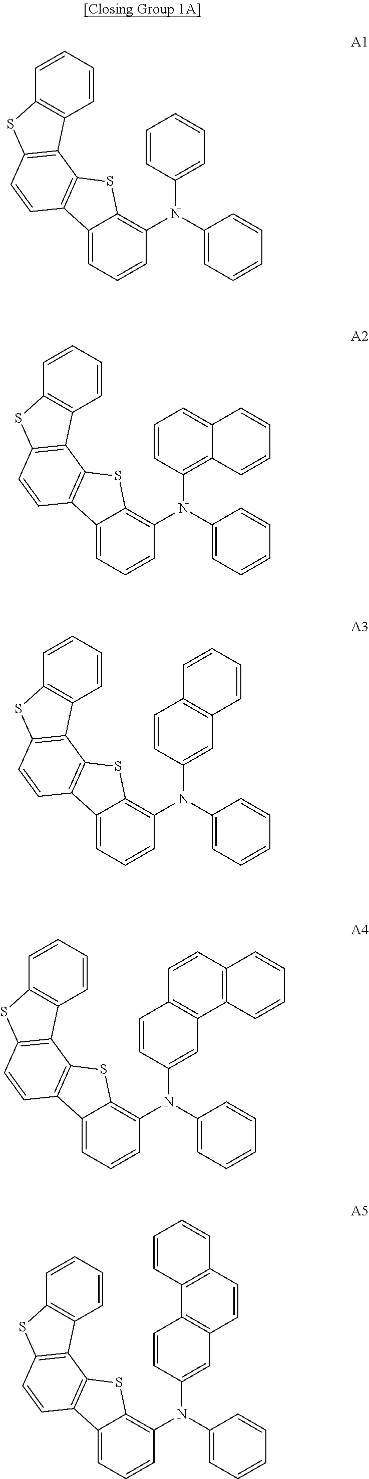 [SMILES: c1ccc(N(c2ccccc2)c2cccc3c2sc2c3ccc3sc4ccccc4c32)cc1 ; c1ccc(N(c2cccc3ccccc23)c2cccc3c2sc2c3ccc3sc4ccccc4c32)cc1 ; c1ccc(N(c2ccc3ccccc3c2)c2cccc3c2sc2c3ccc3sc4ccccc4c32)cc1 ; c1ccc(N(c2ccc3ccc4ccccc4c3c2)c2cccc3c2sc2c3ccc3sc4ccccc4c32)cc1 ; c1ccc(N(c2ccc3c(ccc4ccccc43)c2)c2cccc3c2sc2c3ccc3sc4ccccc4c32)cc1]

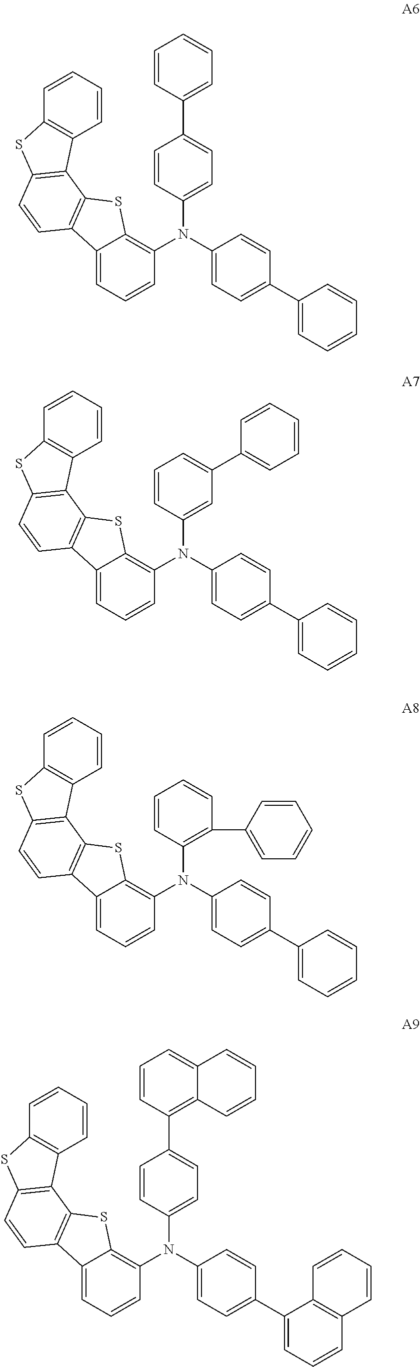 [SMILES: c1ccc(-c2ccc(N(c3ccc(-c4ccccc4)cc3)c3cccc4c3sc3c4ccc4sc5ccccc5c43)cc2)cc1 ; c1ccc(-c2ccc(N(c3cccc(-c4ccccc4)c3)c3cccc4c3sc3c4ccc4sc5ccccc5c43)cc2)cc1 ; c1ccc(-c2ccc(N(c3ccccc3-c3ccccc3)c3cccc4c3sc3c4ccc4sc5ccccc5c43)cc2)cc1 ; c1ccc2c(-c3ccc(N(c4ccc(-c5cccc6ccccc56)cc4)c4cccc5c4sc4c5ccc5sc6ccccc6c54)cc3)cccc2c1]

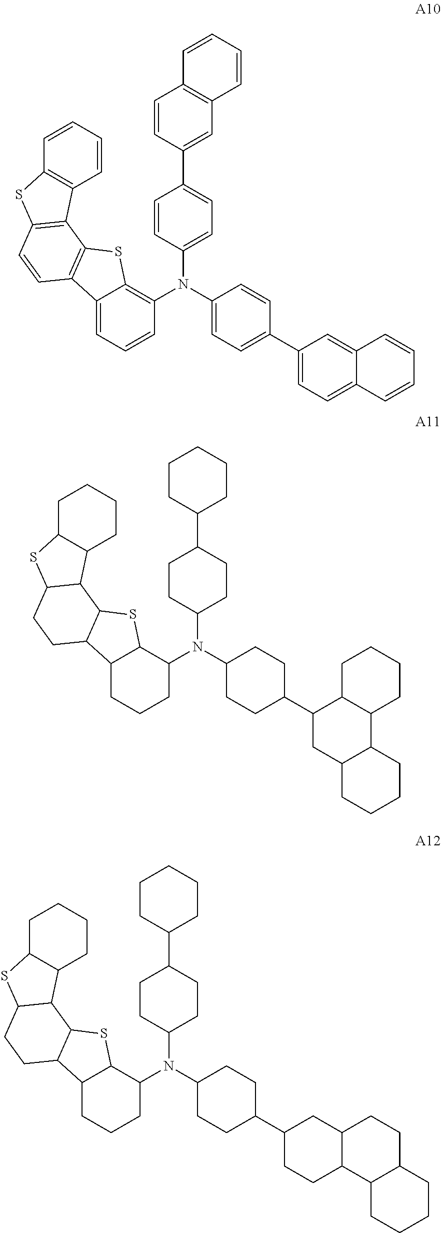 [SMILES: c1ccc2cc(-c3ccc(N(c4ccc(-c5ccc6ccccc6c5)cc4)c4cccc5c4sc4c5ccc5sc6ccccc6c54)cc3)ccc2c1 ; C1CCC(C2CCC(N(C3CCC(C4CC5CCCCC5C5CCCCC45)CC3)C3CCCC4C5CCC6SC7CCCCC7C6C5SC43)CC2)CC1 ; C1CCC(C2CCC(N(C3CCC(C4CCC5C(CCC6CCCCC65)C4)CC3)C3CCCC4C5CCC6SC7CCCCC7C6C5SC43)CC2)CC1]

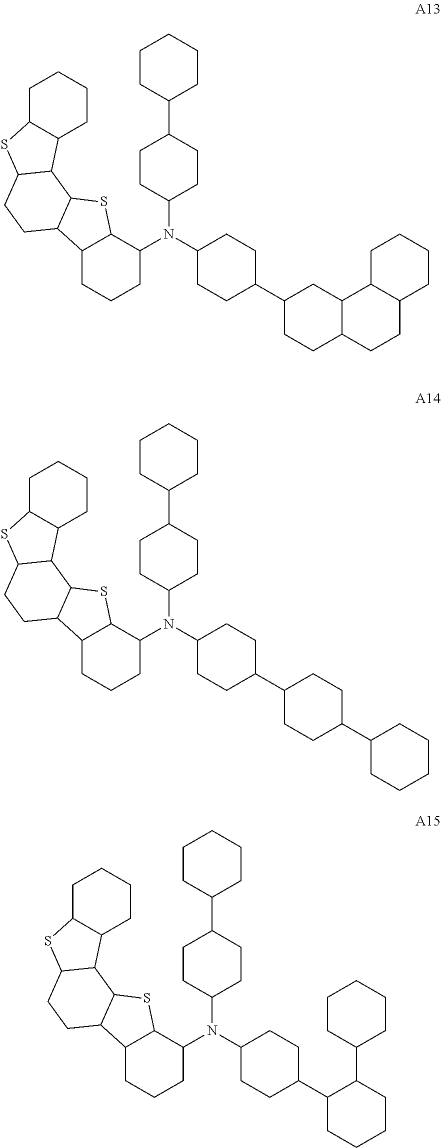 [SMILES: C1CCC(C2CCC(N(C3CCC(C4CCC5CCC6CCCCC6C5C4)CC3)C3CCCC4C5CCC6SC7CCCCC7C6C5SC43)CC2)CC1 ; C1CCC(C2CCC(C3CCC(N(C4CCC(C5CCCCC5)CC4)C4CCCC5C6CCC7SC8CCCCC8C7C6SC54)CC3)CC2)CC1 ; C1CCC(C2CCC(N(C3CCC(C4CCCCC4C4CCCCC4)CC3)C3CCCC4C5CCC6SC7CCCCC7C6C5SC43)CC2)CC1]

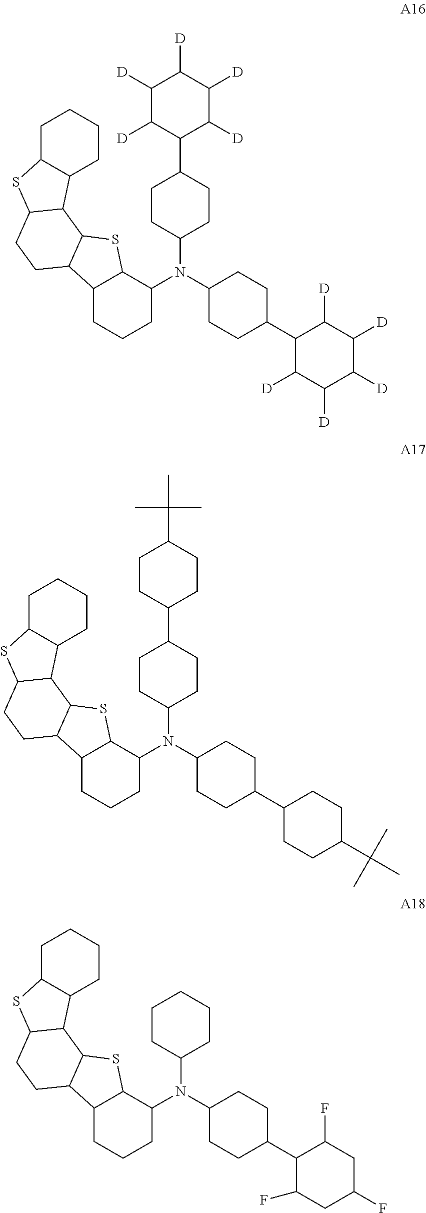 [SMILES: [2H]C1C([2H])C([2H])C(C2CCC(N(C3CCC(C4C([2H])C([2H])C([2H])C([2H])C4[2H])CC3)C3CCCC4C5CCC6SC7CCCCC7C6C5SC43)CC2)C([2H])C1[2H] ; CC(C)(C)C1CCC(C2CCC(N(C3CCC(C4CCC(C(C)(C)C)CC4)CC3)C3CCCC4C5CCC6SC7CCCCC7C6C5SC43)CC2)CC1 ; FC1CC(F)C(C2CCC(N(C3CCCCC3)C3CCCC4C5CCC6SC7CCCCC7C6C5SC43)CC2)C(F)C1]

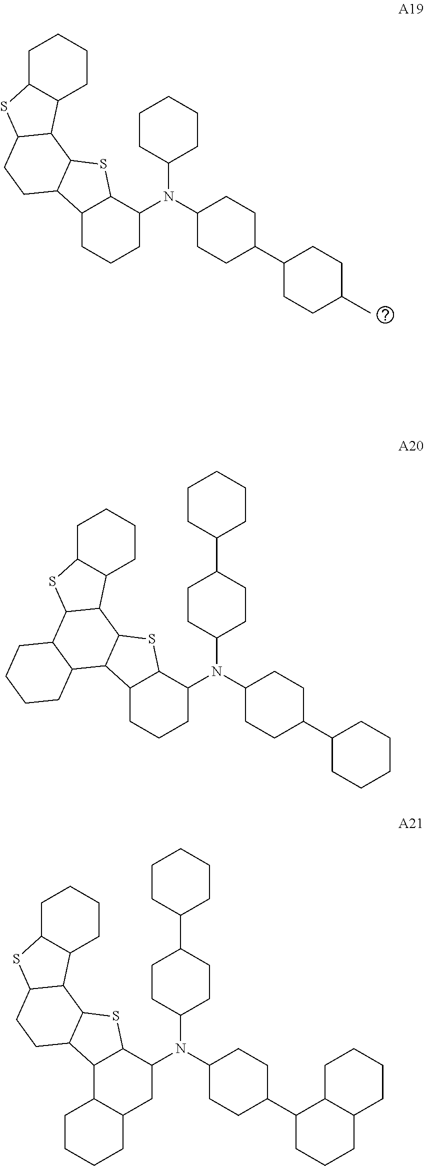 [SMILES: CC1CCC(C2CCC(N(C3CCCCC3)C3CCCC4C5CCC6SC7CCCCC7C6C5SC43)CC2)CC1 ; C1CCC(C2CCC(N(C3CCC(C4CCCCC4)CC3)C3CCCC4C3SC3C4C4CCCCC4C4SC5CCCCC5C43)CC2)CC1 ; C1CCC(C2CCC(N(C3CCC(C4CCCC5CCCCC54)CC3)C3CC4CCCCC4C4C5CCC6SC7CCCCC7C6C5SC43)CC2)CC1]

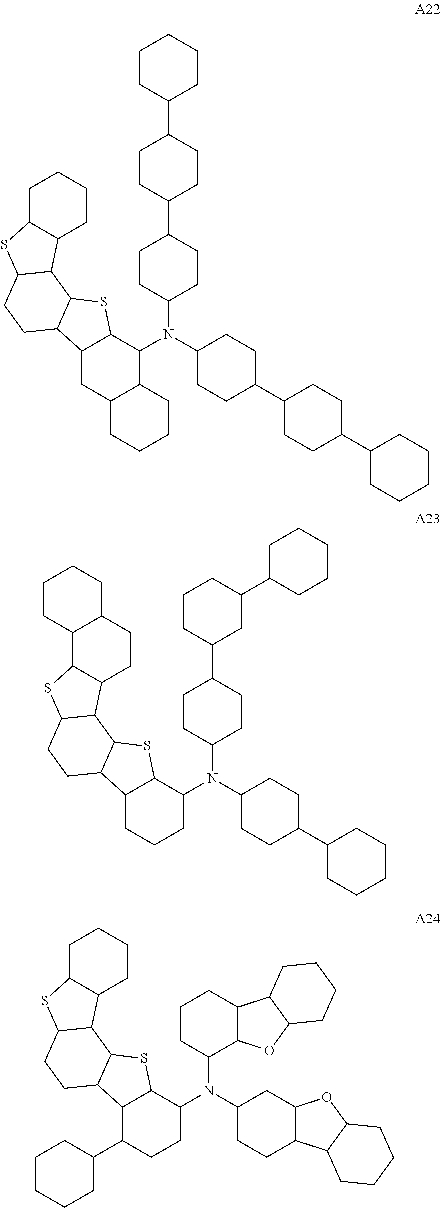 [SMILES: C1CCC(C2CCC(C3CCC(N(C4CCC(C5CCC(C6CCCCC6)CC5)CC4)C4C5CCCCC5CC5C6CCC7SC8CCCCC8C7C6SC54)CC3)CC2)CC1 ; C1CCC(C2CCC(N(C3CCC(C4CCCC(C5CCCCC5)C4)CC3)C3CCCC4C5CCC6SC7C8CCCCC8CCC7C6C5SC43)CC2)CC1 ; C1CCC(C2CCC(N(C3CCC4C(C3)OC3CCCCC34)C3CCCC4C5CCCCC5OC43)C3SC4C(CCC5SC6CCCCC6C54)C23)CC1]

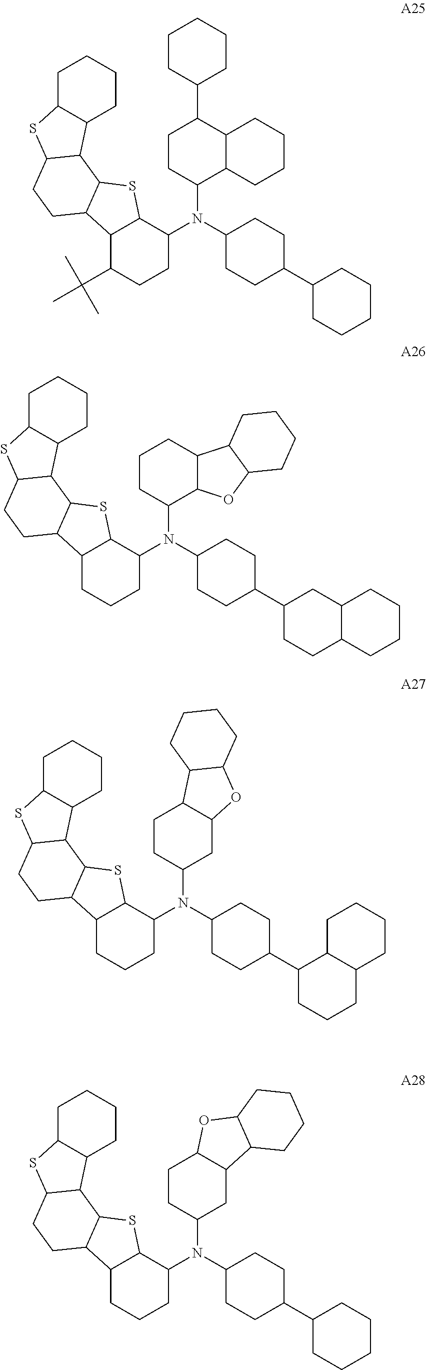 [SMILES: CC(C)(C)C1CCC(N(C2CCC(C3CCCCC3)CC2)C2CCC(C3CCCCC3)C3CCCCC32)C2SC3C(CCC4SC5CCCCC5C43)C21 ; C1CCC2CC(C3CCC(N(C4CCCC5C6CCCCC6OC54)C4CCCC5C6CCC7SC8CCCCC8C7C6SC54)CC3)CCC2C1 ; C1CCC2C(C1)CCCC2C1CCC(N(C2CCC3C(C2)OC2CCCCC23)C2CCCC3C4CCC5SC6CCCCC6C5C4SC32)CC1 ; C1CCC(C2CCC(N(C3CCC4OC5CCCCC5C4C3)C3CCCC4C5CCC6SC7CCCCC7C6C5SC43)CC2)CC1]

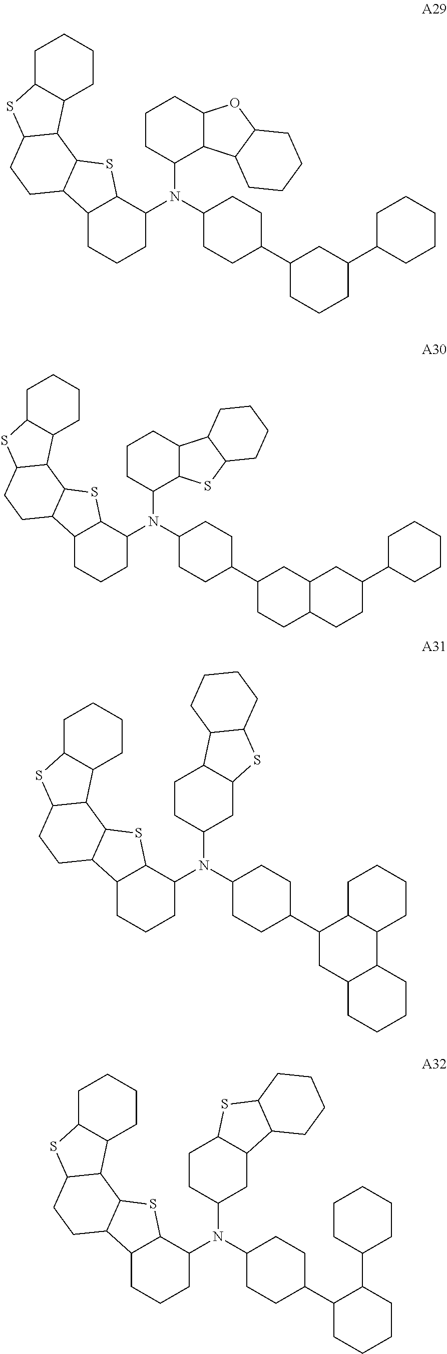 [SMILES: C1CCC(C2CCCC(C3CCC(N(C4CCCC5C6CCC7SC8CCCCC8C7C6SC54)C4CCCC5OC6CCCCC6C54)CC3)C2)CC1 ; C1CCC(C2CCC3CCC(C4CCC(N(C5CCCC6C7CCCCC7SC65)C5CCCC6C7CCC8SC9CCCCC9C8C7SC65)CC4)CC3C2)CC1 ; C1CCC2C(C1)CC(C1CCC(N(C3CCC4C(C3)SC3CCCCC34)C3CCCC4C5CCC6SC7CCCCC7C6C5SC43)CC1)C1CCCCC21 ; C1CCC(C2CCCCC2C2CCC(N(C3CCC4SC5CCCCC5C4C3)C3CCCC4C5CCC6SC7CCCCC7C6C5SC43)CC2)CC1]

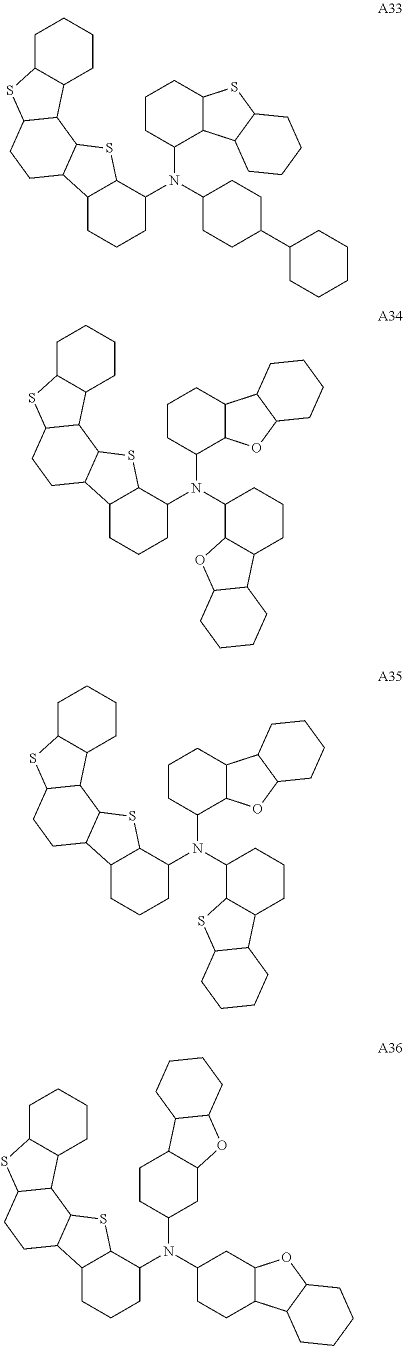 [SMILES: C1CCC(C2CCC(N(C3CCCC4C5CCC6SC7CCCCC7C6C5SC43)C3CCCC4SC5CCCCC5C43)CC2)CC1 ; C1CCC2C(C1)OC1C2CCCC1N(C1CCCC2C3CCCCC3OC21)C1CCCC2C3CCC4SC5CCCCC5C4C3SC21 ; C1CCC2C(C1)OC1C2CCCC1N(C1CCCC2C3CCCCC3SC21)C1CCCC2C3CCC4SC5CCCCC5C4C3SC21 ; C1CCC2C(C1)OC1CC(N(C3CCC4C(C3)OC3CCCCC34)C3CCCC4C5CCC6SC7CCCCC7C6C5SC43)CCC12]

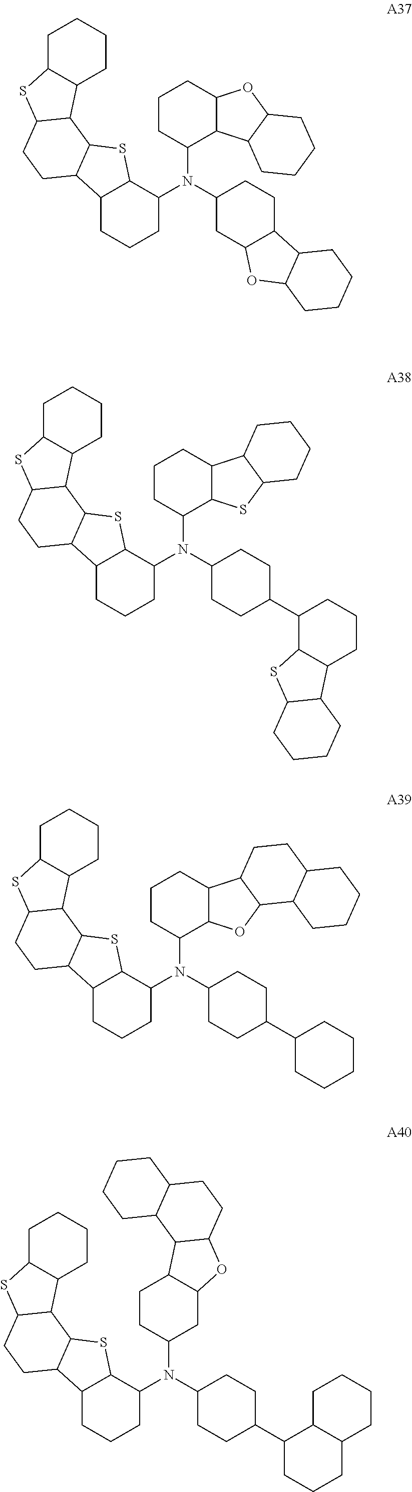 [SMILES: C1CCC2C(C1)OC1CC(N(C3CCCC4C5CCC6SC7CCCCC7C6C5SC43)C3CCCC4OC5CCCCC5C43)CCC12 ; C1CCC2C(C1)SC1C(C3CCC(N(C4CCCC5C6CCCCC6SC54)C4CCCC5C6CCC7SC8CCCCC8C7C6SC54)CC3)CCCC21 ; C1CCC(C2CCC(N(C3CCCC4C5CCC6CCCCC6C5OC43)C3CCCC4C5CCC6SC7CCCCC7C6C5SC43)CC2)CC1 ; C1CCC2C(C1)CCCC2C1CCC(N(C2CCC3C(C2)OC2CCC4CCCCC4C23)C2CCCC3C4CCC5SC6CCCCC6C5C4SC32)CC1]

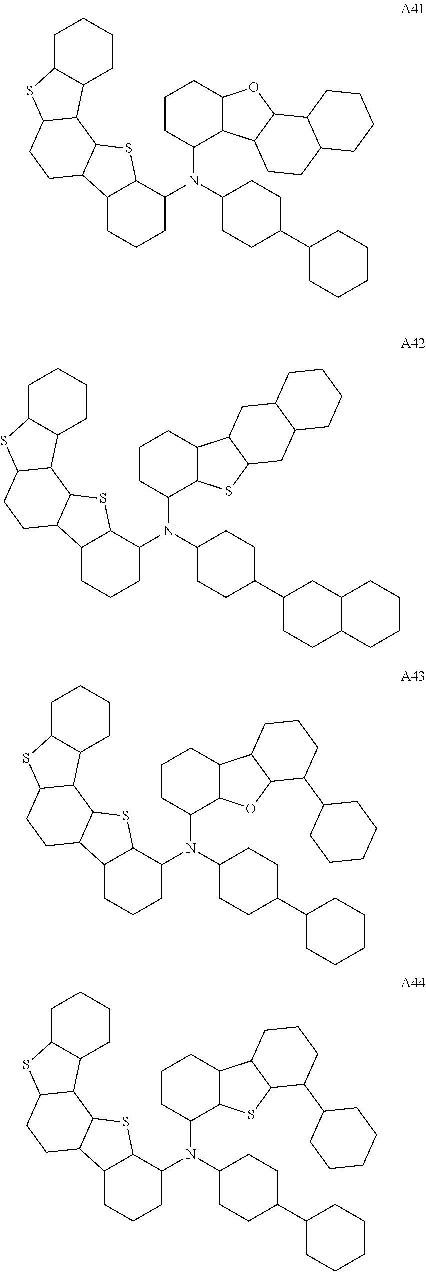 [SMILES: C1CCC(C2CCC(N(C3CCCC4C5CCC6SC7CCCCC7C6C5SC43)C3CCCC4OC5C6CCCCC6CCC5C43)CC2)CC1 ; C1CCC2CC(C3CCC(N(C4CCCC5C6CC7CCCCC7CC6SC54)C4CCCC5C6CCC7SC8CCCCC8C7C6SC54)CC3)CCC2C1 ; C1CCC(C2CCC(N(C3CCCC4C5CCCC(C6CCCCC6)C5OC43)C3CCCC4C5CCC6SC7CCCCC7C6C5SC43)CC2)CC1 ; C1CCC(C2CCC(N(C3CCCC4C5CCCC(C6CCCCC6)C5SC43)C3CCCC4C5CCC6SC7CCCCC7C6C5SC43)CC2)CC1]

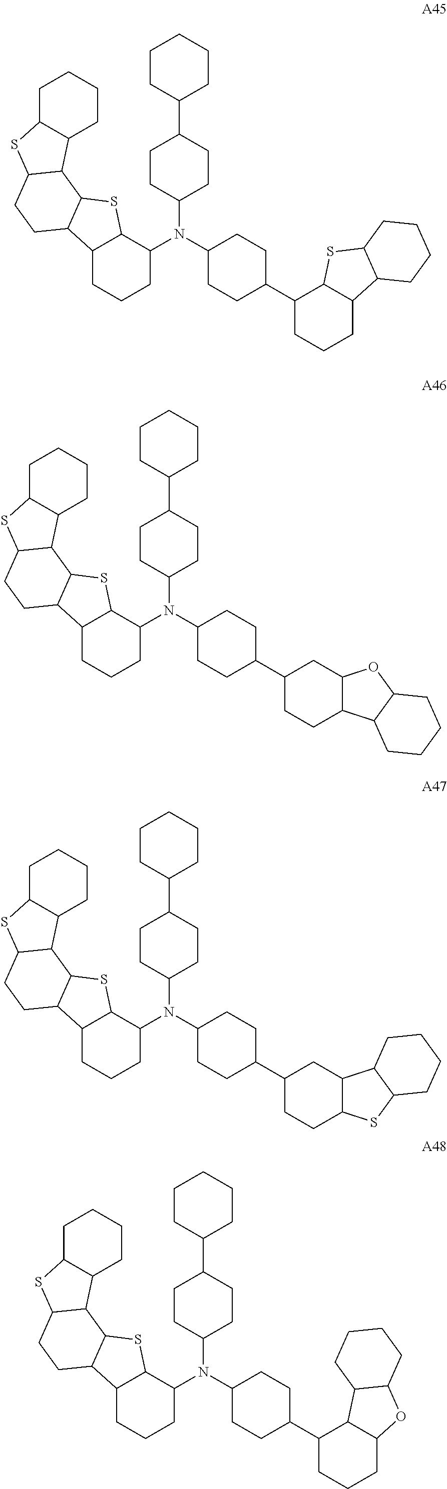 [SMILES: C1CCC(C2CCC(N(C3CCC(C4CCCC5C6CCCCC6SC45)CC3)C3CCCC4C5CCC6SC7CCCCC7C6C5SC43)CC2)CC1 ; C1CCC(C2CCC(N(C3CCC(C4CCC5C(C4)OC4CCCCC45)CC3)C3CCCC4C5CCC6SC7CCCCC7C6C5SC43)CC2)CC1 ; C1CCC(C2CCC(N(C3CCC(C4CCC5SC6CCCCC6C5C4)CC3)C3CCCC4C5CCC6SC7CCCCC7C6C5SC43)CC2)CC1 ; C1CCC(C2CCC(N(C3CCC(C4CCCC5OC6CCCCC6C54)CC3)C3CCCC4C5CCC6SC7CCCCC7C6C5SC43)CC2)CC1]

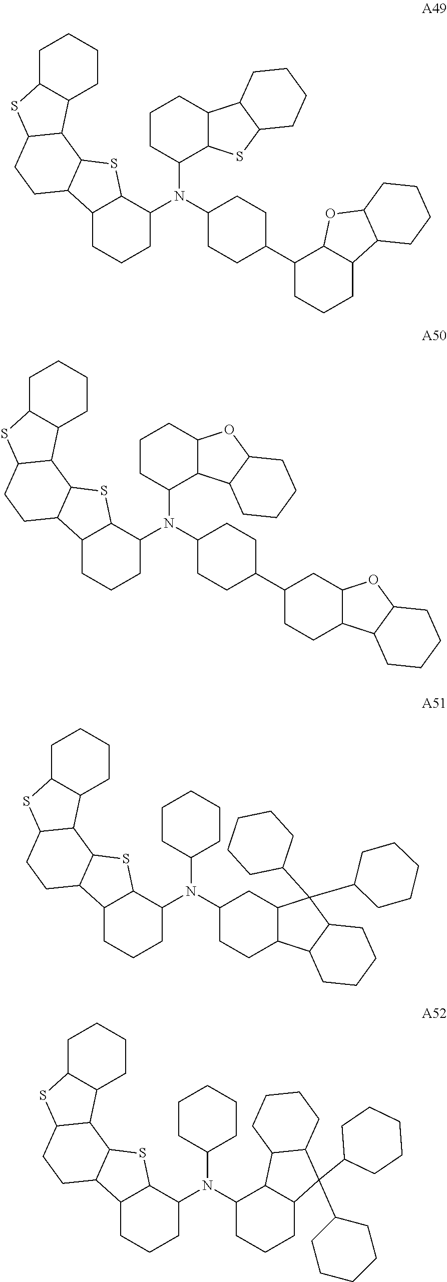 [SMILES: C1CCC2C(C1)OC1C(C3CCC(N(C4CCCC5C6CCCCC6SC54)C4CCCC5C6CCC7SC8CCCCC8C7C6SC54)CC3)CCCC21 ; C1CCC2C(C1)OC1CC(C3CCC(N(C4CCCC5C6CCC7SC8CCCCC8C7C6SC54)C4CCCC5OC6CCCCC6C54)CC3)CCC12 ; C1CCC(N(C2CCC3C4CCCCC4C(C4CCCCC4)(C4CCCCC4)C3C2)C2CCCC3C4CCC5SC6CCCCC6C5C4SC32)CC1 ; C1CCC(N(C2CCCC3C4CCC5SC6CCCCC6C5C4SC32)C2CCCC3C2C2CCCCC2C3(C2CCCCC2)C2CCCCC2)CC1]

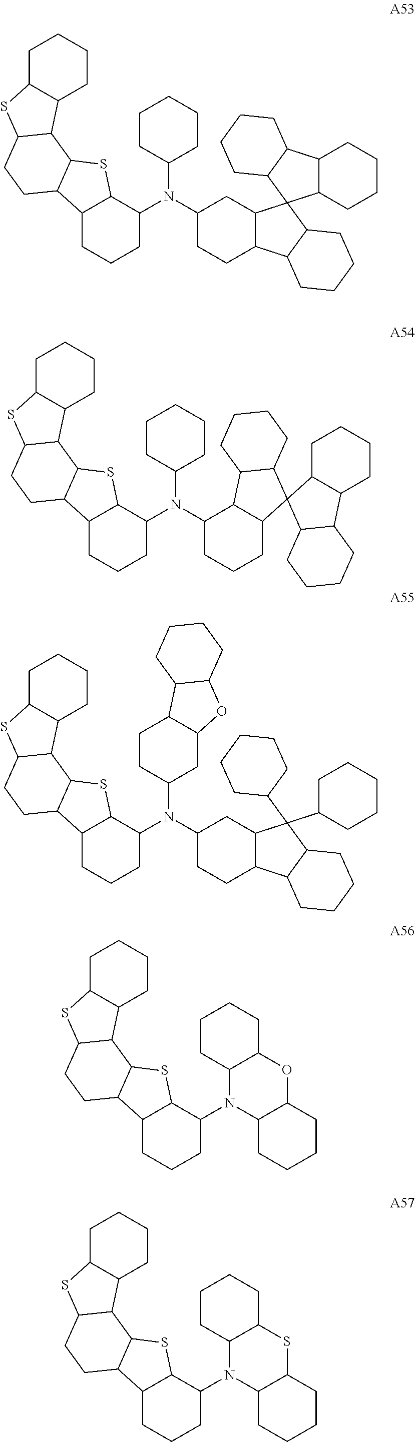 [SMILES: C1CCC(N(C2CCC3C4CCCCC4C4(C5CCCCC5C5CCCCC54)C3C2)C2CCCC3C4CCC5SC6CCCCC6C5C4SC32)CC1 ; C1CCC(N(C2CCCC3C4CCC5SC6CCCCC6C5C4SC32)C2CCCC3C2C2CCCCC2C32C3CCCCC3C3CCCCC32)CC1 ; C1CCC(C2(C3CCCCC3)C3CCCCC3C3CCC(N(C4CCC5C(C4)OC4CCCCC45)C4CCCC5C6CCC7SC8CCCCC8C7C6SC54)CC32)CC1 ; C1CCC2C(C1)OC1CCCCC1N2C1CCCC2C3CCC4SC5CCCCC5C4C3SC21 ; C1CCC2C(C1)SC1CCC3C4CCCC(N5C6CCCCC6SC6CCCCC65)C4SC3C12]

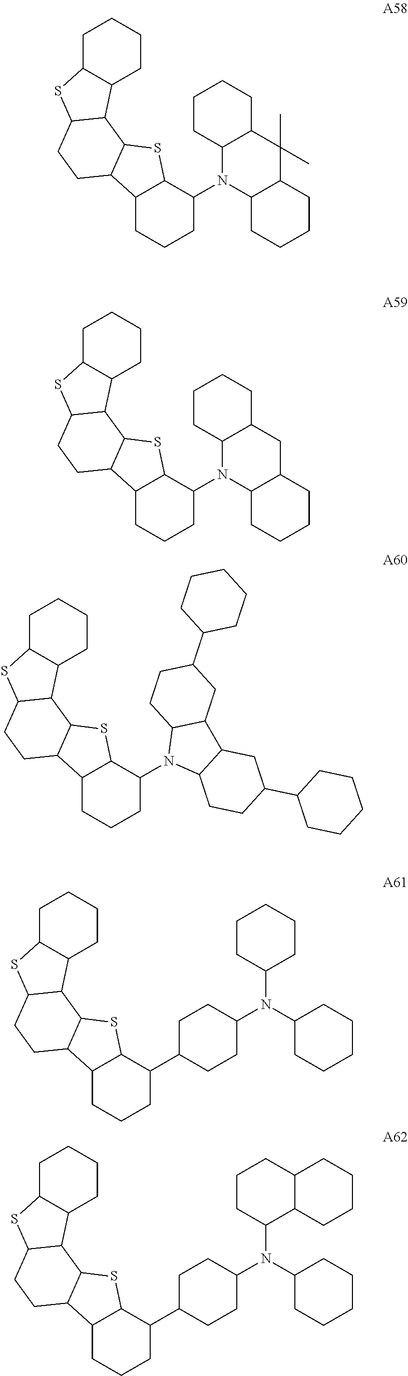

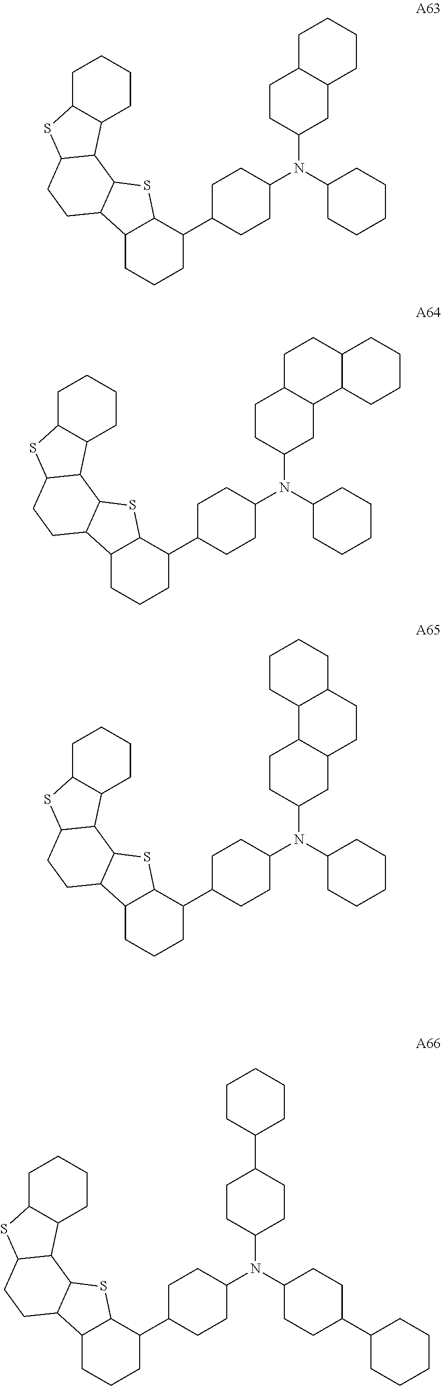 [SMILES: C1CCC(N(C2CCC(C3CCCC4C5CCC6SC7CCCCC7C6C5SC34)CC2)C2CCC3CCCCC3C2)CC1 ; C1CCC(N(C2CCC(C3CCCC4C5CCC6SC7CCCCC7C6C5SC34)CC2)C2CCC3CCC4CCCCC4C3C2)CC1 ; C1CCC(N(C2CCC(C3CCCC4C5CCC6SC7CCCCC7C6C5SC34)CC2)C2CCC3C(CCC4CCCCC43)C2)CC1 ; C1CCC(C2CCC(N(C3CCC(C4CCCCC4)CC3)C3CCC(C4CCCC5C6CCC7SC8CCCCC8C7C6SC45)CC3)CC2)CC1]

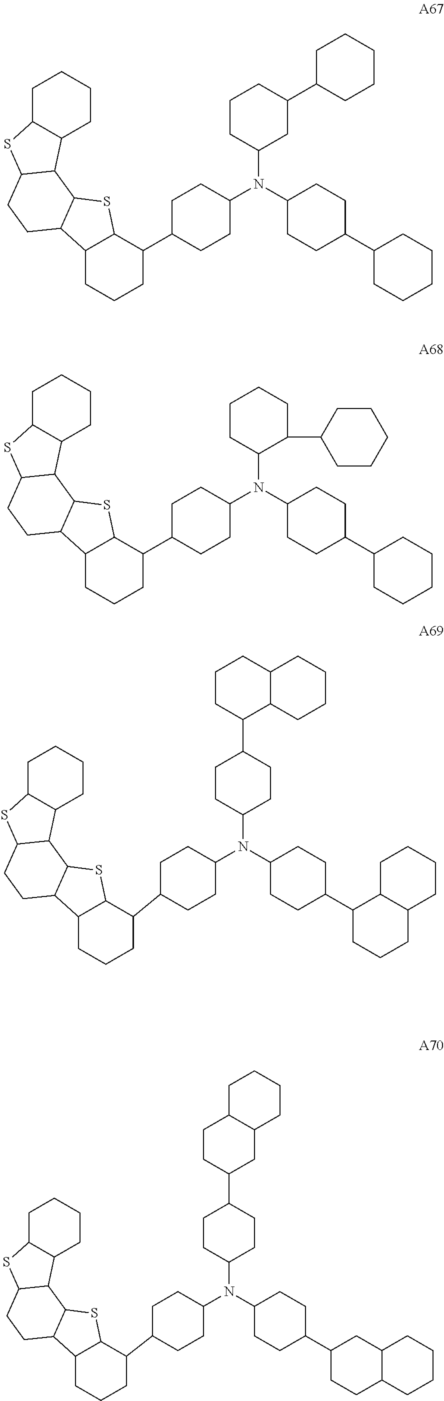 [SMILES: C1CCC(C2CCC(N(C3CCC(C4CCCC5C6CCC7SC8CCCCC8C7C6SC45)CC3)C3CCCC(C4CCCCC4)C3)CC2)CC1 ; C1CCC(C2CCC(N(C3CCC(C4CCCC5C6CCC7SC8CCCCC8C7C6SC45)CC3)C3CCCCC3C3CCCCC3)CC2)CC1 ; C1CCC2C(C1)CCCC2C1CCC(N(C2CCC(C3CCCC4CCCCC43)CC2)C2CCC(C3CCCC4C5CCC6SC7CCCCC7C6C5SC34)CC2)CC1 ; C1CCC2CC(C3CCC(N(C4CCC(C5CCC6CCCCC6C5)CC4)C4CCC(C5CCCC6C7CCC8SC9CCCCC9C8C7SC56)CC4)CC3)CCC2C1]

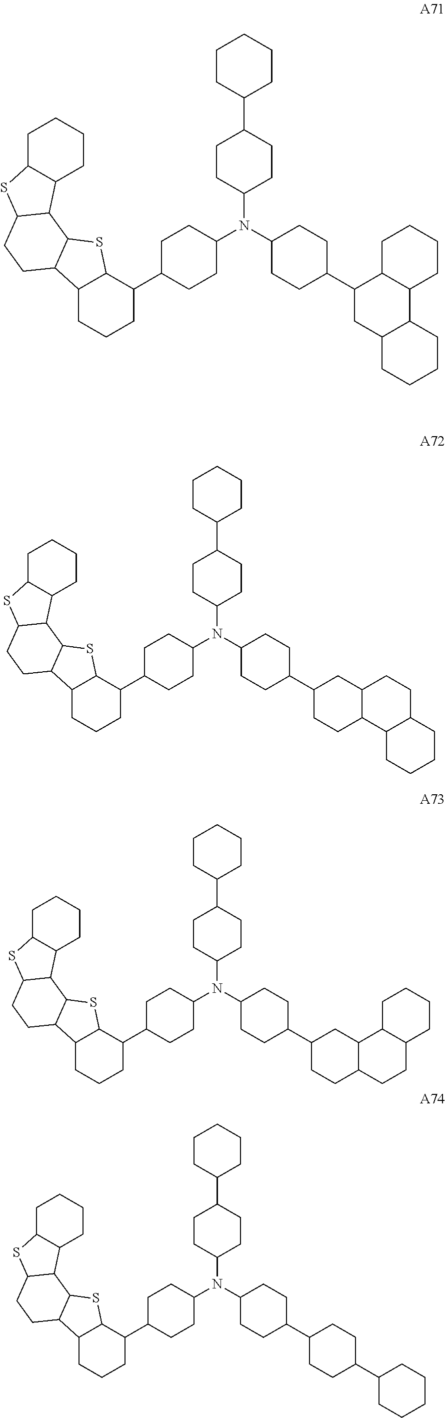 [SMILES: C1CCC(C2CCC(N(C3CCC(C4CC5CCCCC5C5CCCCC45)CC3)C3CCC(C4CCCC5C6CCC7SC8CCCCC8C7C6SC45)CC3)CC2)CC1 ; C1CCC(C2CCC(N(C3CCC(C4CCC5C(CCC6CCCCC65)C4)CC3)C3CCC(C4CCCC5C6CCC7SC8CCCCC8C7C6SC45)CC3)CC2)CC1 ; C1CCC(C2CCC(N(C3CCC(C4CCC5CCC6CCCCC6C5C4)CC3)C3CCC(C4CCCC5C6CCC7SC8CCCCC8C7C6SC45)CC3)CC2)CC1 ; C1CCC(C2CCC(C3CCC(N(C4CCC(C5CCCCC5)CC4)C4CCC(C5CCCC6C7CCC8SC9CCCCC9C8C7SC56)CC4)CC3)CC2)CC1]

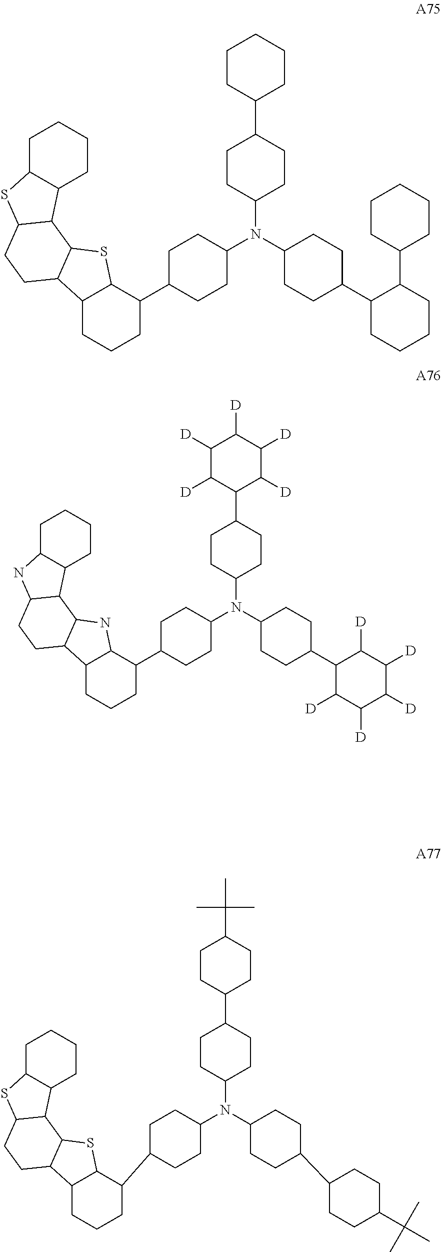 [SMILES: C1CCC(C2CCC(N(C3CCC(C4CCCCC4C4CCCCC4)CC3)C3CCC(C4CCCC5C6CCC7SC8CCCCC8C7C6SC45)CC3)CC2)CC1 ; [2H]C1C([2H])C([2H])C(C2CCC(N(C3CCC(C4CCCC5C6CCC7NC8CCCCC8C7C6NC45)CC3)C3CCC(C4C([2H])C([2H])C([2H])C([2H])C4[2H])CC3)CC2)C([2H])C1[2H] ; CC(C)(C)C1CCC(C2CCC(N(C3CCC(C4CCC(C(C)(C)C)CC4)CC3)C3CCC(C4CCCC5C6CCC7SC8CCCCC8C7C6SC45)CC3)CC2)CC1]

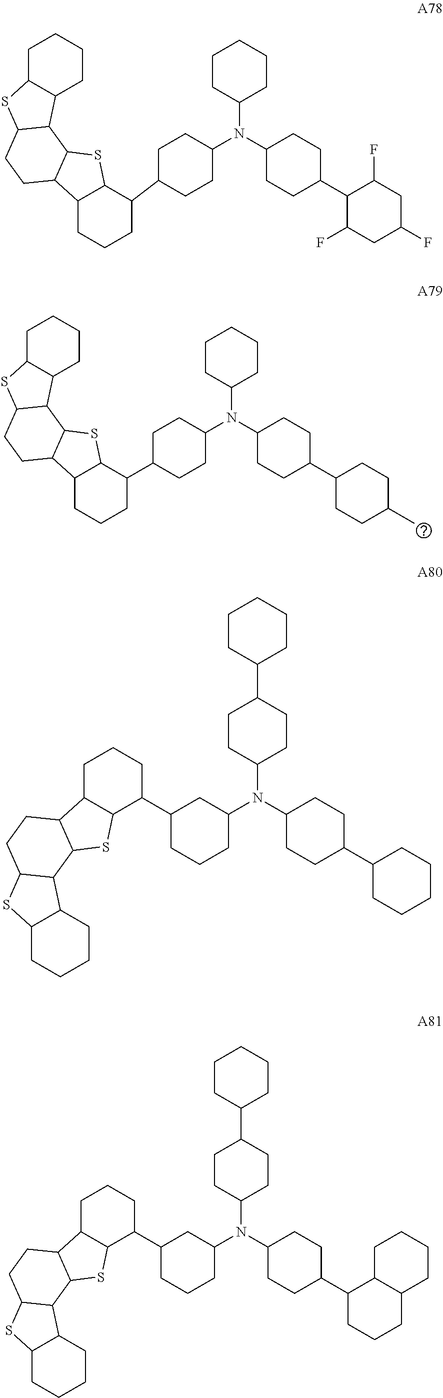 [SMILES: FC1CC(F)C(C2CCC(N(C3CCCCC3)C3CCC(C4CCCC5C6CCC7SC8CCCCC8C7C6SC45)CC3)CC2)C(F)C1 ; CC1CCC(C2CCC(N(C3CCCCC3)C3CCC(C4CCCC5C6CCC7SC8CCCCC8C7C6SC45)CC3)CC2)CC1 ; C1CCC(C2CCC(N(C3CCC(C4CCCCC4)CC3)C3CCCC(C4CCCC5C6CCC7SC8CCCCC8C7C6SC45)C3)CC2)CC1 ; C1CCC(C2CCC(N(C3CCC(C4CCCC5CCCCC54)CC3)C3CCCC(C4CCCC5C6CCC7SC8CCCCC8C7C6SC45)C3)CC2)CC1]

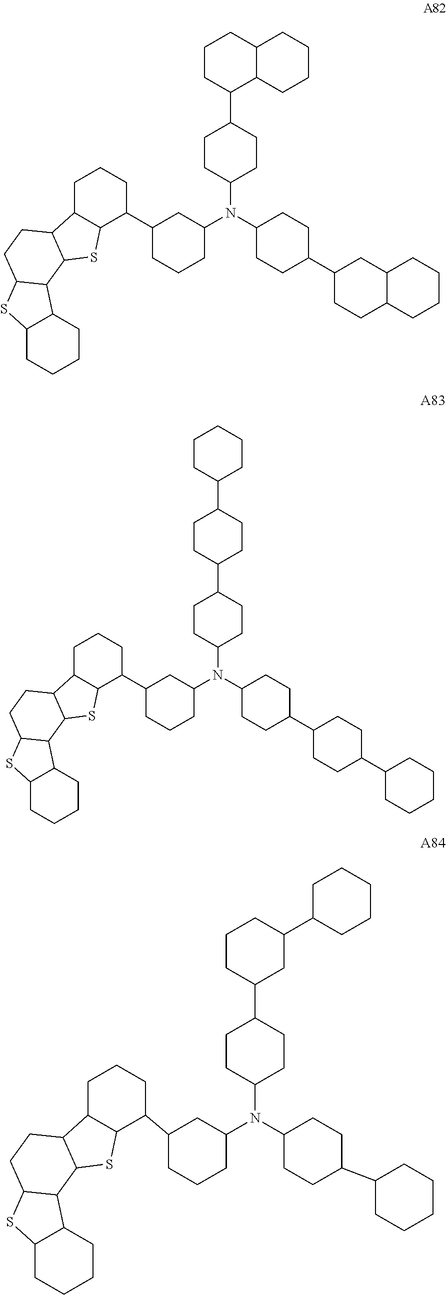 [SMILES: C1CCC2CC(C3CCC(N(C4CCC(C5CCCC6CCCCC65)CC4)C4CCCC(C5CCCC6C7CCC8SC9CCCCC9C8C7SC56)C4)CC3)CCC2C1 ; C1CCC(C2CCC(C3CCC(N(C4CCC(C5CCC(C6CCCCC6)CC5)CC4)C4CCCC(C5CCCC6C7CCC8SC9CCCCC9C8C7SC56)C4)CC3)CC2)CC1 ; C1CCC(C2CCC(N(C3CCC(C4CCCC(C5CCCCC5)C4)CC3)C3CCCC(C4CCCC5C6CCC7SC8CCCCC8C7C6SC45)C3)CC2)CC1]

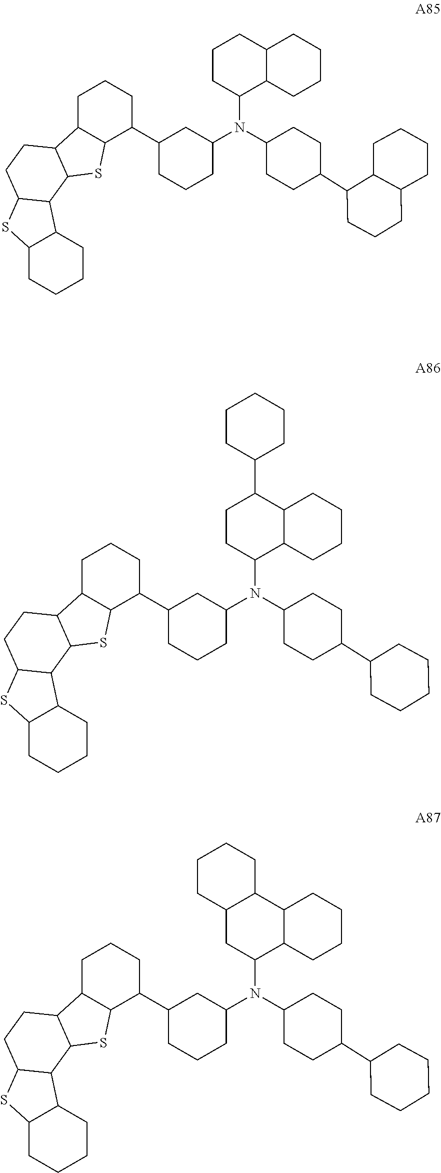 [SMILES: C1CCC2C(C1)CCCC2C1CCC(N(C2CCCC(C3CCCC4C5CCC6SC7CCCCC7C6C5SC34)C2)C2CCCC3CCCCC32)CC1 ; C1CCC(C2CCC(N(C3CCCC(C4CCCC5C6CCC7SC8CCCCC8C7C6SC45)C3)C3CCC(C4CCCCC4)C4CCCCC43)CC2)CC1 ; C1CCC(C2CCC(N(C3CCCC(C4CCCC5C6CCC7SC8CCCCC8C7C6SC45)C3)C3CC4CCCCC4C4CCCCC43)CC2)CC1]

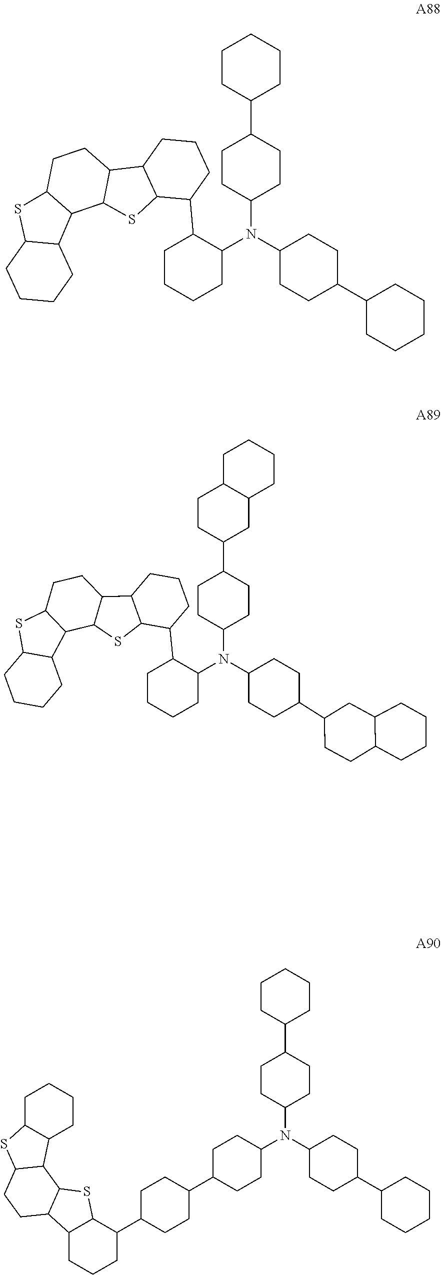 [SMILES: C1CCC(C2CCC(N(C3CCC(C4CCCCC4)CC3)C3CCCCC3C3CCCC4C5CCC6SC7CCCCC7C6C5SC43)CC2)CC1 ; C1CCC2CC(C3CCC(N(C4CCC(C5CCC6CCCCC6C5)CC4)C4CCCCC4C4CCCC5C6CCC7SC8CCCCC8C7C6SC54)CC3)CCC2C1 ; C1CCC(C2CCC(N(C3CCC(C4CCCCC4)CC3)C3CCC(C4CCC(C5CCCC6C7CCC8SC9CCCCC9C8C7SC56)CC4)CC3)CC2)CC1]

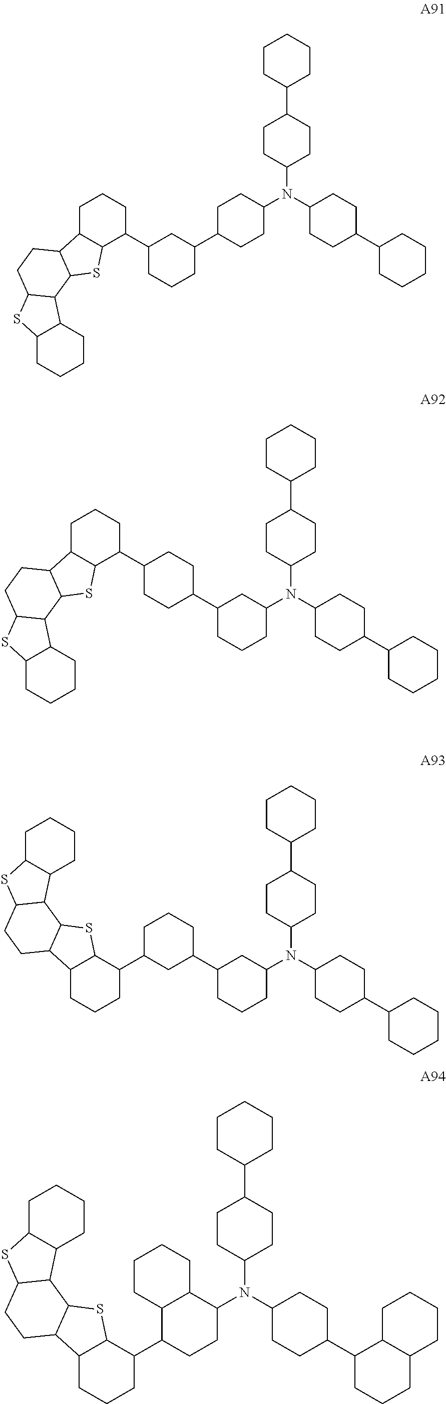 [SMILES: C1CCC(C2CCC(N(C3CCC(C4CCCCC4)CC3)C3CCC(C4CCCC(C5CCCC6C7CCC8SC9CCCCC9C8C7SC56)C4)CC3)CC2)CC1 ; C1CCC(C2CCC(N(C3CCC(C4CCCCC4)CC3)C3CCCC(C4CCC(C5CCCC6C7CCC8SC9CCCCC9C8C7SC56)CC4)C3)CC2)CC1 ; C1CCC(C2CCC(N(C3CCC(C4CCCCC4)CC3)C3CCCC(C4CCCC(C5CCCC6C7CCC8SC9CCCCC9C8C7SC56)C4)C3)CC2)CC1 ; C1CCC(C2CCC(N(C3CCC(C4CCCC5CCCCC54)CC3)C3CCC(C4CCCC5C6CCC7SC8CCCCC8C7C6SC45)C4CCCCC43)CC2)CC1]

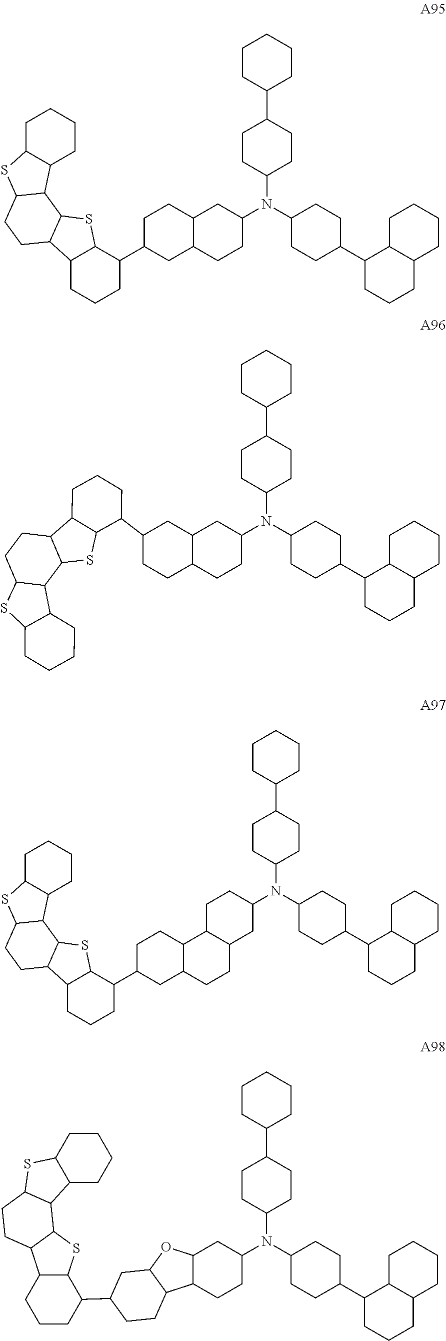 [SMILES: C1CCC(C2CCC(N(C3CCC(C4CCCC5CCCCC54)CC3)C3CCC4CC(C5CCCC6C7CCC8SC9CCCCC9C8C7SC56)CCC4C3)CC2)CC1 ; C1CCC(C2CCC(N(C3CCC(C4CCCC5CCCCC54)CC3)C3CCC4CCC(C5CCCC6C7CCC8SC9CCCCC9C8C7SC56)CC4C3)CC2)CC1 ; C1CCC(C2CCC(N(C3CCC(C4CCCC5CCCCC54)CC3)C3CCC4C(CCC5CC(C6CCCC7C8CCC9SC%10CCCCC%10C9C8SC67)CCC54)C3)CC2)CC1 ; C1CCC(C2CCC(N(C3CCC(C4CCCC5CCCCC54)CC3)C3CCC4C(C3)OC3CC(C5CCCC6C7CCC8SC9CCCCC9C8C7SC56)CCC34)CC2)CC1]

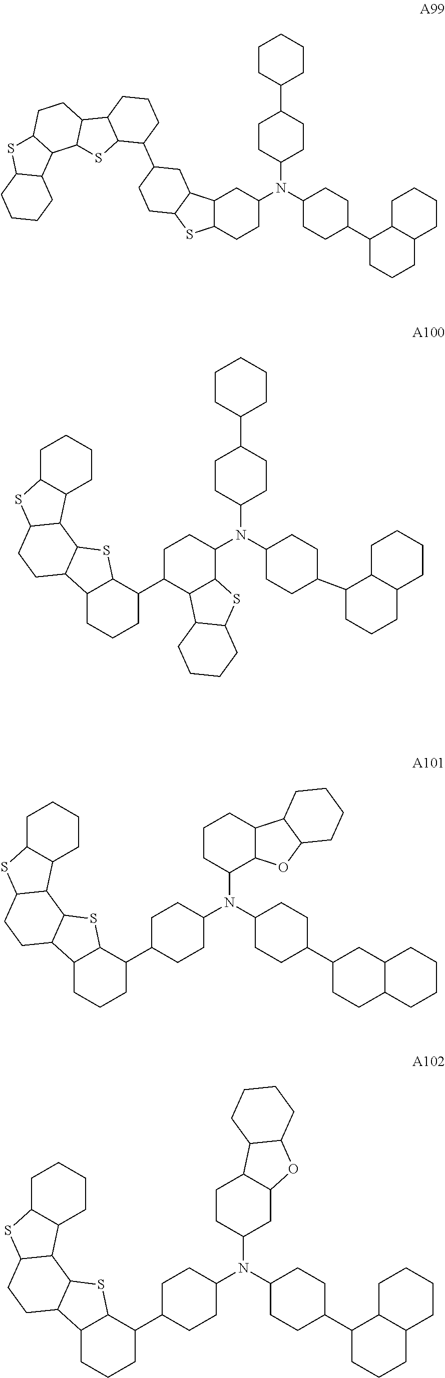 [SMILES: C1CCC(C2CCC(N(C3CCC(C4CCCC5CCCCC54)CC3)C3CCC4SC5CCC(C6CCCC7C8CCC9SC%10CCCCC%10C9C8SC67)CC5C4C3)CC2)CC1 ; C1CCC(C2CCC(N(C3CCC(C4CCCC5CCCCC54)CC3)C3CCC(C4CCCC5C6CCC7SC8CCCCC8C7C6SC54)C4C5CCCCC5SC43)CC2)CC1 ; C1CCC2CC(C3CCC(N(C4CCC(C5CCCC6C7CCC8SC9CCCCC9C8C7SC56)CC4)C4CCCC5C6CCCCC6OC54)CC3)CCC2C1 ; C1CCC2C(C1)CCCC2C1CCC(N(C2CCC(C3CCCC4C5CCC6SC7CCCCC7C6C5SC34)CC2)C2CCC3C(C2)OC2CCCCC23)CC1]

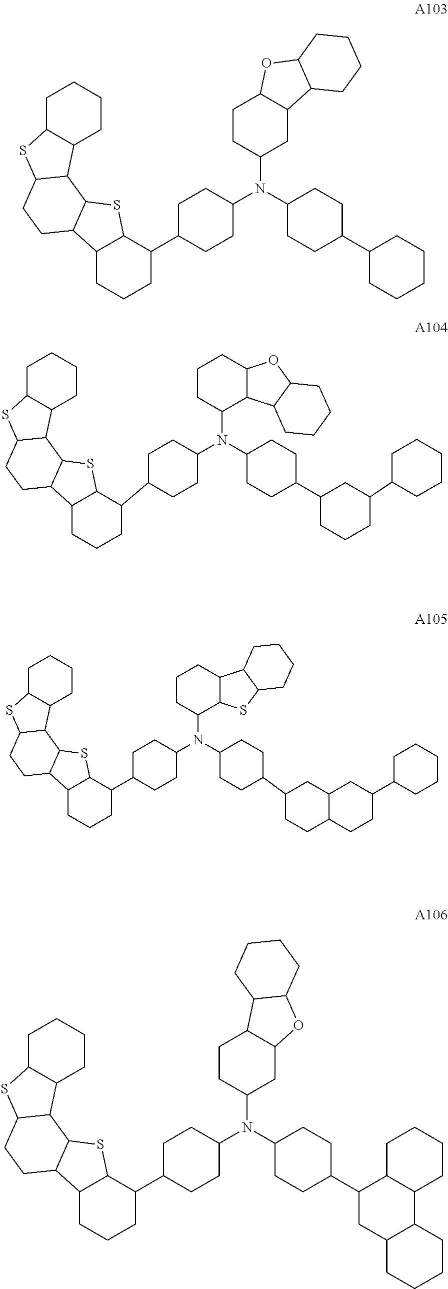 [SMILES: C1CCC(C2CCC(N(C3CCC(C4CCCC5C6CCC7SC8CCCCC8C7C6SC45)CC3)C3CCC4OC5CCCCC5C4C3)CC2)CC1 ; C1CCC(C2CCCC(C3CCC(N(C4CCC(C5CCCC6C7CCC8SC9CCCCC9C8C7SC56)CC4)C4CCCC5OC6CCCCC6C54)CC3)C2)CC1 ; C1CCC(C2CCC3CCC(C4CCC(N(C5CCC(C6CCCC7C8CCC9SC%10CCCCC%10C9C8SC67)CC5)C5CCCC6C7CCCCC7SC65)CC4)CC3C2)CC1 ; C1CCC2C(C1)CC(C1CCC(N(C3CCC(C4CCCC5C6CCC7SC8CCCCC8C7C6SC45)CC3)C3CCC4C(C3)OC3CCCCC34)CC1)C1CCCCC21]

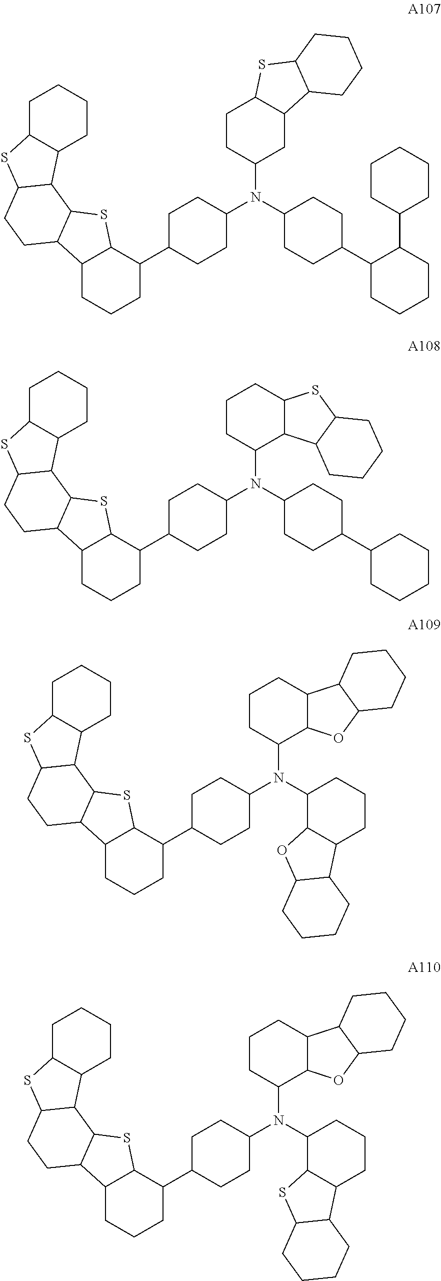 [SMILES: C1CCC(C2CCCCC2C2CCC(N(C3CCC(C4CCCC5C6CCC7SC8CCCCC8C7C6SC45)CC3)C3CCC4SC5CCCCC5C4C3)CC2)CC1 ; C1CCC(C2CCC(N(C3CCC(C4CCCC5C6CCC7SC8CCCCC8C7C6SC45)CC3)C3CCCC4SC5CCCCC5C43)CC2)CC1 ; C1CCC2C(C1)OC1C2CCCC1N(C1CCC(C2CCCC3C4CCC5SC6CCCCC6C5C4SC23)CC1)C1CCCC2C3CCCCC3OC21 ; C1CCC2C(C1)OC1C2CCCC1N(C1CCC(C2CCCC3C4CCC5SC6CCCCC6C5C4SC23)CC1)C1CCCC2C3CCCCC3SC21]

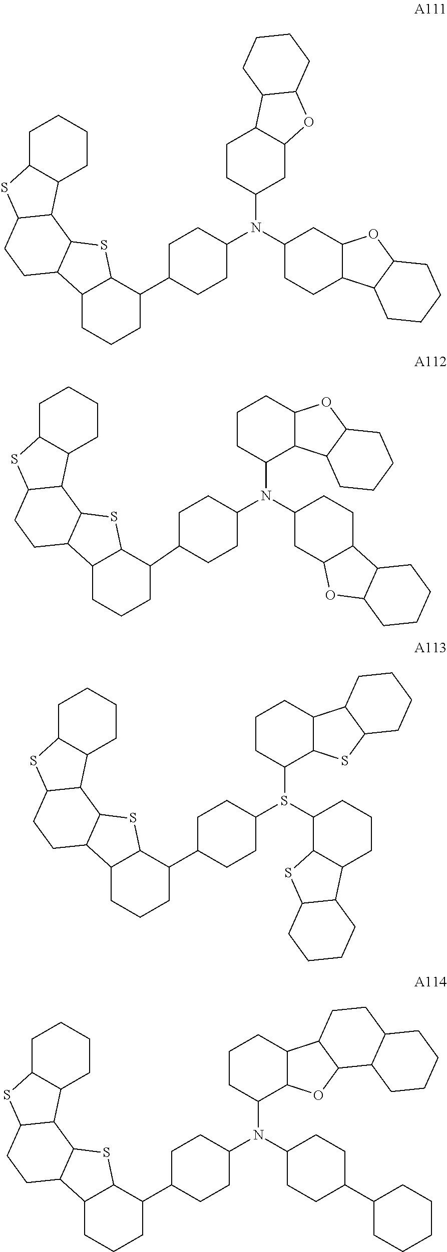

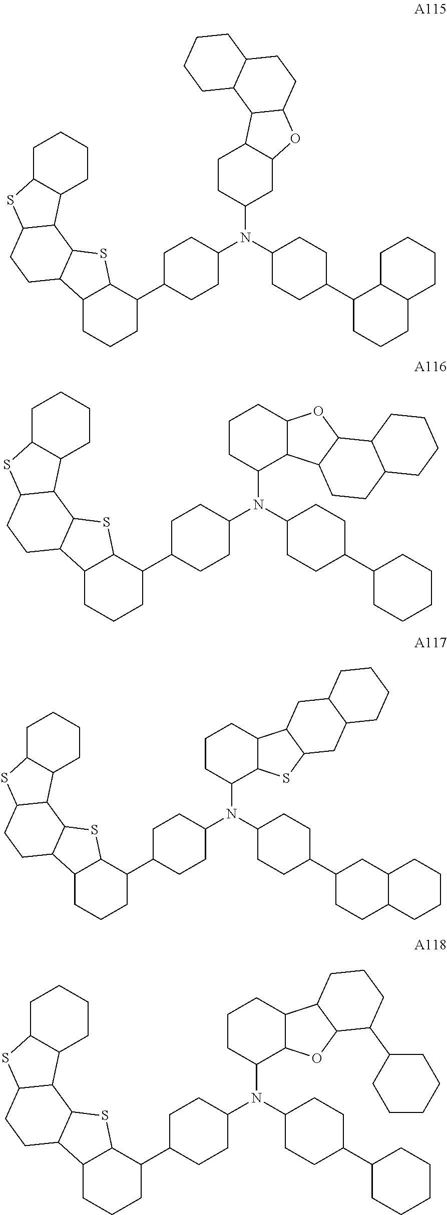 [SMILES: C1CCC2C(C1)CCCC2C1CCC(N(C2CCC(C3CCCC4C5CCC6SC7CCCCC7C6C5SC34)CC2)C2CCC3C(C2)OC2CCC4CCCCC4C23)CC1 ; C1CCC(C2CCC(N(C3CCC(C4CCCC5C6CCC7SC8CCCCC8C7C6SC45)CC3)C3CCCC4OC5C6CCCCC6CCC5C43)CC2)CC1 ; C1CCC2CC(C3CCC(N(C4CCC(C5CCCC6C7CCC8SC9CCCCC9C8C7SC56)CC4)C4CCCC5C6CC7CCCCC7CC6SC54)CC3)CCC2C1 ; C1CCC(C2CCC(N(C3CCC(C4CCCC5C6CCC7SC8CCCCC8C7C6SC45)CC3)C3CCCC4C5CCCC(C6CCCCC6)C5OC43)CC2)CC1]

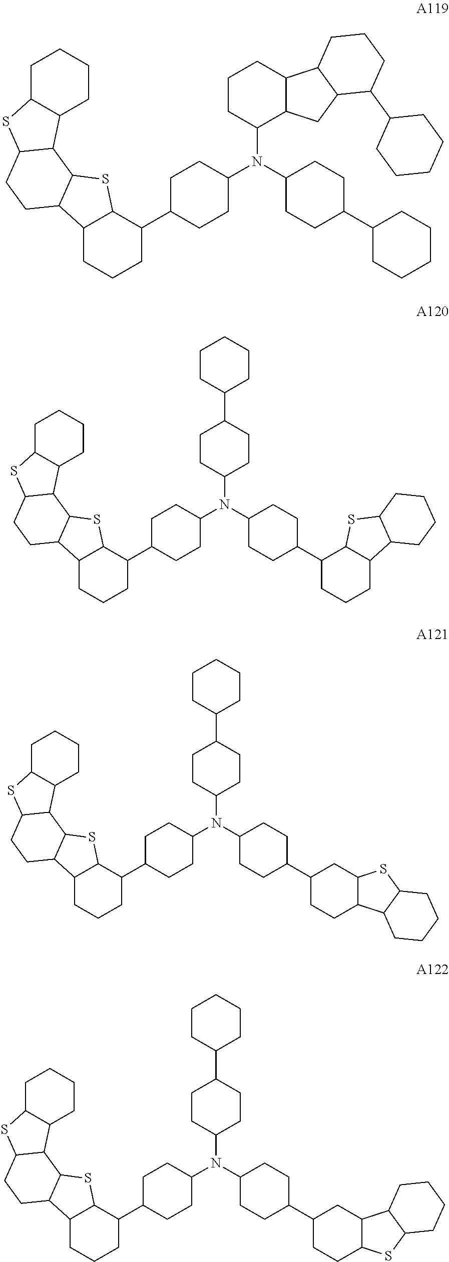 [SMILES: C1CCC(C2CCC(N(C3CCC(C4CCCC5C6CCC7SC8CCCCC8C7C6SC45)CC3)C3CCCC4C5CCCC(C6CCCCC6)C5CC43)CC2)CC1 ; C1CCC(C2CCC(N(C3CCC(C4CCCC5C6CCCCC6SC45)CC3)C3CCC(C4CCCC5C6CCC7SC8CCCCC8C7C6SC45)CC3)CC2)CC1 ; C1CCC(C2CCC(N(C3CCC(C4CCC5C(C4)SC4CCCCC45)CC3)C3CCC(C4CCCC5C6CCC7SC8CCCCC8C7C6SC45)CC3)CC2)CC1 ; C1CCC(C2CCC(N(C3CCC(C4CCC5SC6CCCCC6C5C4)CC3)C3CCC(C4CCCC5C6CCC7SC8CCCCC8C7C6SC45)CC3)CC2)CC1]

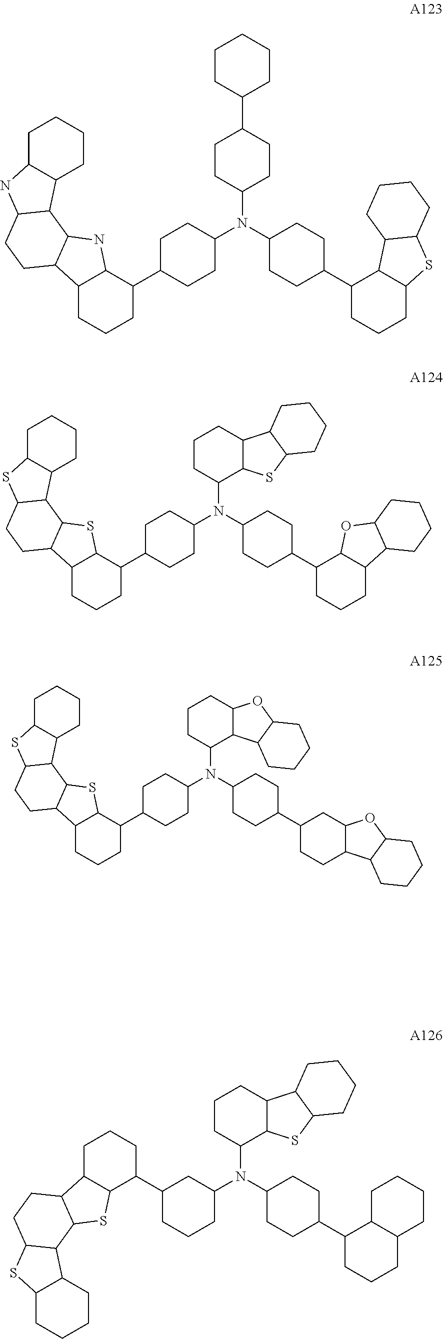 [SMILES: C1CCC(C2CCC(N(C3CCC(C4CCCC5C6CCC7NC8CCCCC8C7C6NC45)CC3)C3CCC(C4CCCC5SC6CCCCC6C54)CC3)CC2)CC1 ; C1CCC2C(C1)OC1C(C3CCC(N(C4CCC(C5CCCC6C7CCC8SC9CCCCC9C8C7SC56)CC4)C4CCCC5C6CCCCC6SC54)CC3)CCCC21 ; C1CCC2C(C1)OC1CC(C3CCC(N(C4CCC(C5CCCC6C7CCC8SC9CCCCC9C8C7SC56)CC4)C4CCCC5OC6CCCCC6C54)CC3)CCC12 ; C1CCC2C(C1)CCCC2C1CCC(N(C2CCCC(C3CCCC4C5CCC6SC7CCCCC7C6C5SC34)C2)C2CCCC3C4CCCCC4SC32)CC1]

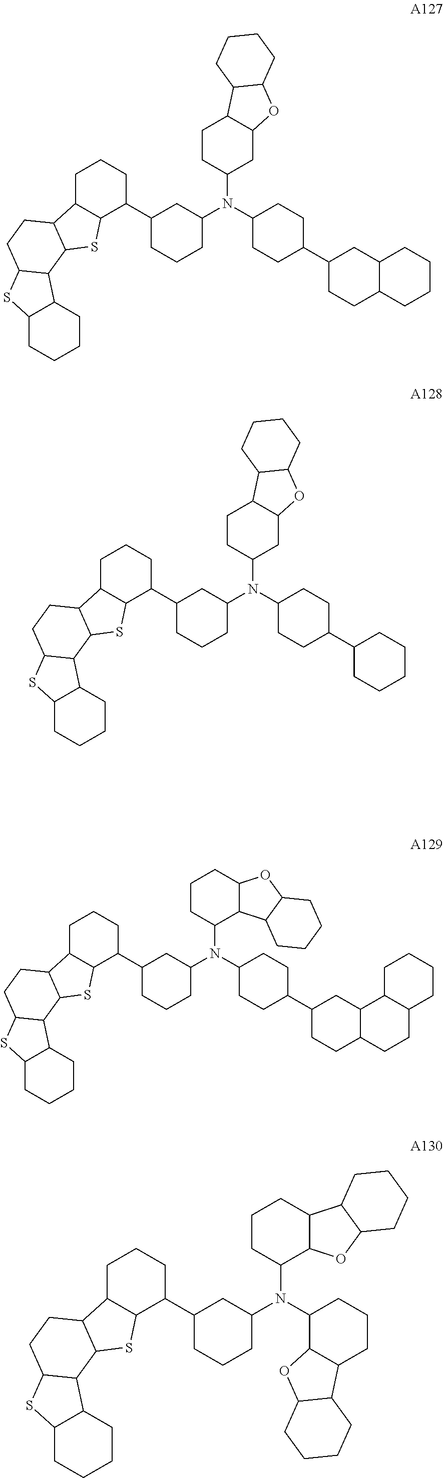 [SMILES: C1CCC2CC(C3CCC(N(C4CCCC(C5CCCC6C7CCC8SC9CCCCC9C8C7SC56)C4)C4CCC5C(C4)OC4CCCCC45)CC3)CCC2C1 ; C1CCC(C2CCC(N(C3CCCC(C4CCCC5C6CCC7SC8CCCCC8C7C6SC45)C3)C3CCC4C(C3)OC3CCCCC34)CC2)CC1 ; C1CCC2C(C1)CCC1CCC(C3CCC(N(C4CCCC(C5CCCC6C7CCC8SC9CCCCC9C8C7SC56)C4)C4CCCC5OC6CCCCC6C54)CC3)CC12 ; C1CCC2C(C1)OC1C2CCCC1N(C1CCCC(C2CCCC3C4CCC5SC6CCCCC6C5C4SC23)C1)C1CCCC2C3CCCCC3OC21]

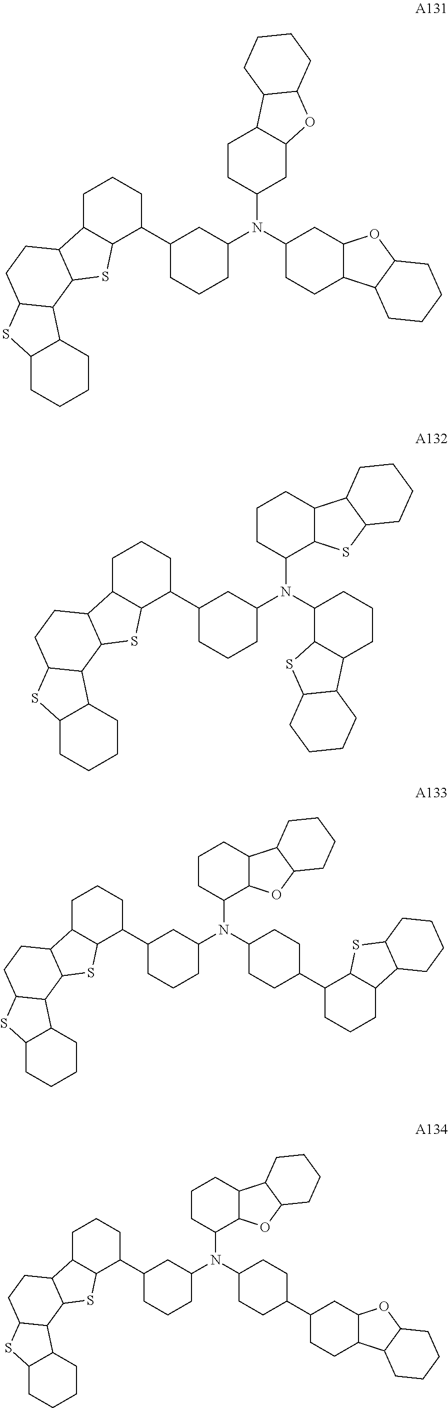 [SMILES: C1CCC2C(C1)OC1CC(N(C3CCCC(C4CCCC5C6CCC7SC8CCCCC8C7C6SC45)C3)C3CCC4C(C3)OC3CCCCC34)CCC12 ; C1CCC2C(C1)SC1C2CCCC1N(C1CCCC(C2CCCC3C4CCC5SC6CCCCC6C5C4SC23)C1)C1CCCC2C3CCCCC3SC21 ; C1CCC2C(C1)OC1C2CCCC1N(C1CCC(C2CCCC3C4CCCCC4SC23)CC1)C1CCCC(C2CCCC3C4CCC5SC6CCCCC6C5C4SC23)C1 ; C1CCC2C(C1)OC1CC(C3CCC(N(C4CCCC(C5CCCC6C7CCC8SC9CCCCC9C8C7SC56)C4)C4CCCC5C6CCCCC6OC54)CC3)CCC12]

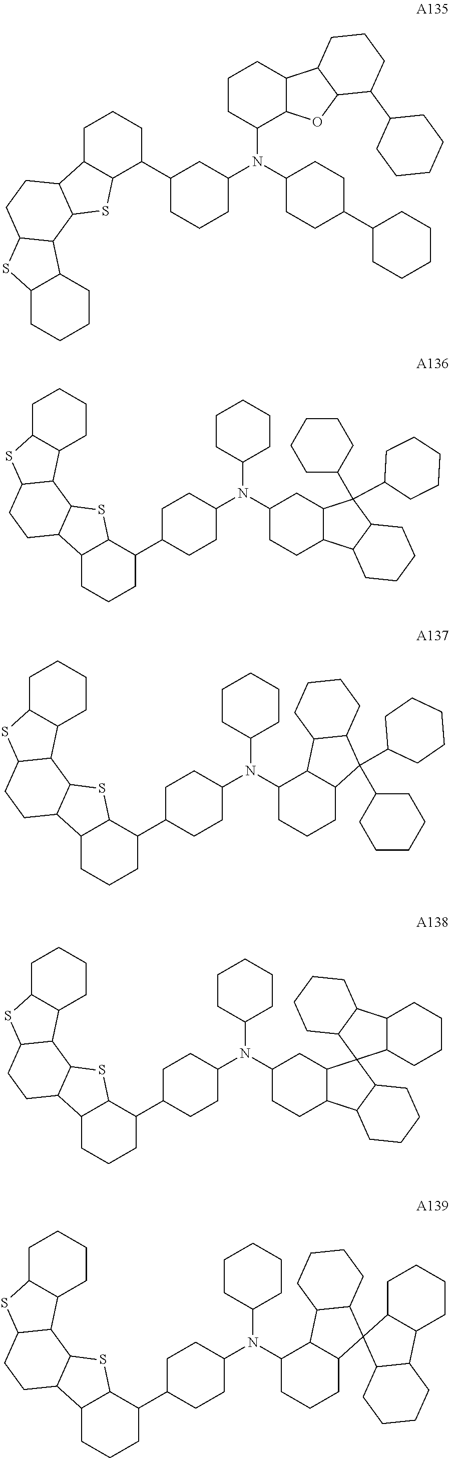 [SMILES: C1CCC(C2CCC(N(C3CCCC(C4CCCC5C6CCC7SC8CCCCC8C7C6SC45)C3)C3CCCC4C5CCCC(C6CCCCC6)C5OC43)CC2)CC1 ; C1CCC(N(C2CCC(C3CCCC4C5CCC6SC7CCCCC7C6C5SC34)CC2)C2CCC3C4CCCCC4C(C4CCCCC4)(C4CCCCC4)C3C2)CC1 ; C1CCC(N(C2CCC(C3CCCC4C5CCC6SC7CCCCC7C6C5SC34)CC2)C2CCCC3C2C2CCCCC2C3(C2CCCCC2)C2CCCCC2)CC1 ; C1CCC(N(C2CCC(C3CCCC4C5CCC6SC7CCCCC7C6C5SC34)CC2)C2CCC3C4CCCCC4C4(C5CCCCC5C5CCCCC54)C3C2)CC1 ; C1CCC(N(C2CCC(C3CCCC4C5CCC6SC7CCCCC7C6C5SC34)CC2)C2CCCC3C2C2CCCCC2C32C3CCCCC3C3CCCCC32)CC1]

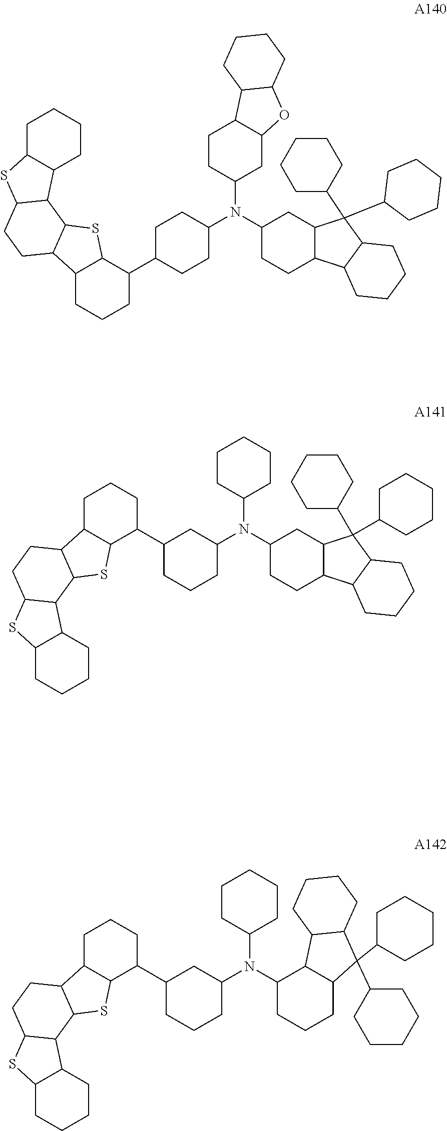 [SMILES: C1CCC(C2(C3CCCCC3)C3CCCCC3C3CCC(N(C4CCC(C5CCCC6C7CCC8SC9CCCCC9C8C7SC56)CC4)C4CCC5C(C4)OC4CCCCC45)CC32)CC1 ; C1CCC(N(C2CCCC(C3CCCC4C5CCC6SC7CCCCC7C6C5SC34)C2)C2CCC3C4CCCCC4C(C4CCCCC4)(C4CCCCC4)C3C2)CC1 ; C1CCC(N(C2CCCC(C3CCCC4C5CCC6SC7CCCCC7C6C5SC34)C2)C2CCCC3C2C2CCCCC2C3(C2CCCCC2)C2CCCCC2)CC1]

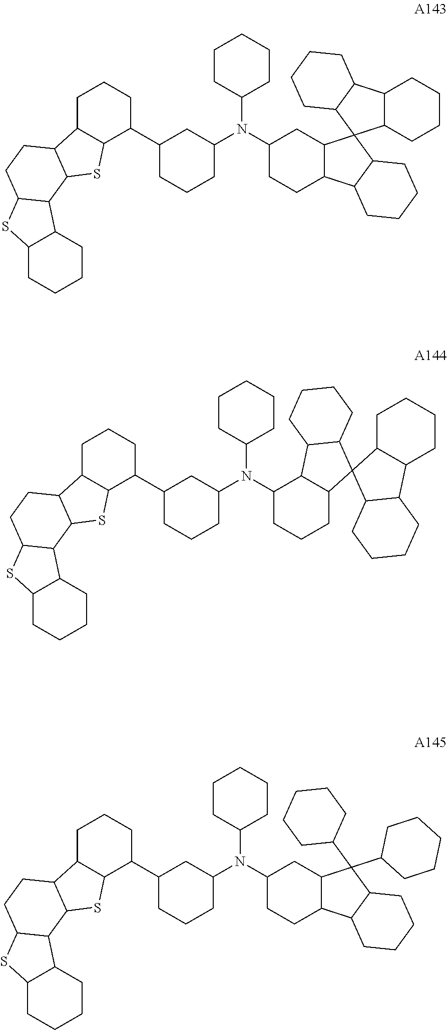 [SMILES: C1CCC(N(C2CCCC(C3CCCC4C5CCC6SC7CCCCC7C6C5SC34)C2)C2CCC3C4CCCCC4C4(C5CCCCC5C5CCCCC54)C3C2)CC1 ; C1CCC(N(C2CCCC(C3CCCC4C5CCC6SC7CCCCC7C6C5SC34)C2)C2CCCC3C2C2CCCCC2C32C3CCCCC3C3CCCCC32)CC1 ; C1CCC(N(C2CCCC(C3CCCC4C5CCC6SC7CCCCC7C6C5SC34)C2)C2CCC3C4CCCCC4C(C4CCCCC4)(C4CCCCC4)C3C2)CC1]

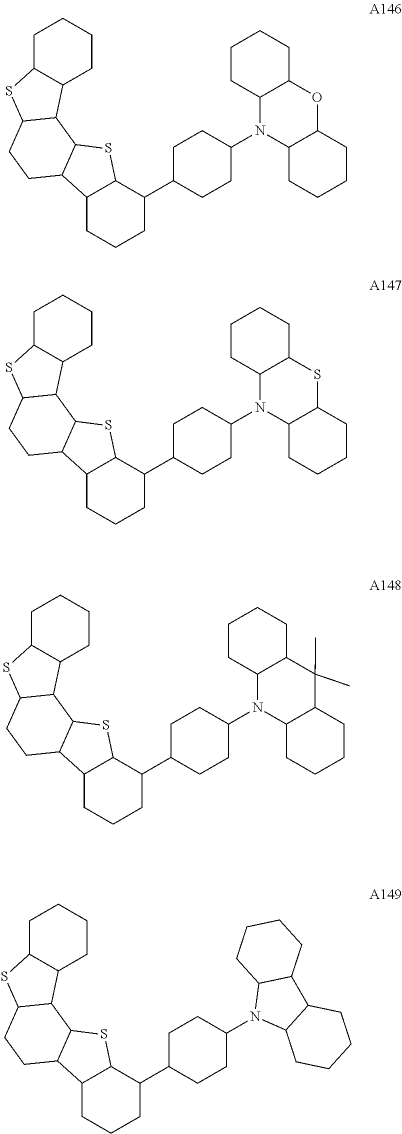 [SMILES: C1CCC2C(C1)OC1CCCCC1N2C1CCC(C2CCCC3C4CCC5SC6CCCCC6C5C4SC23)CC1 ; C1CCC2C(C1)SC1CCC3C4CCCC(C5CCC(N6C7CCCCC7SC7CCCCC76)CC5)C4SC3C12 ; CC1(C)C2CCCCC2N(C2CCC(C3CCCC4C5CCC6SC7CCCCC7C6C5SC34)CC2)C2CCCCC21 ; C1CCC2C(C1)SC1CCC3C4CCCC(C5CCC(N6C7CCCCC7C7CCCCC76)CC5)C4SC3C12]

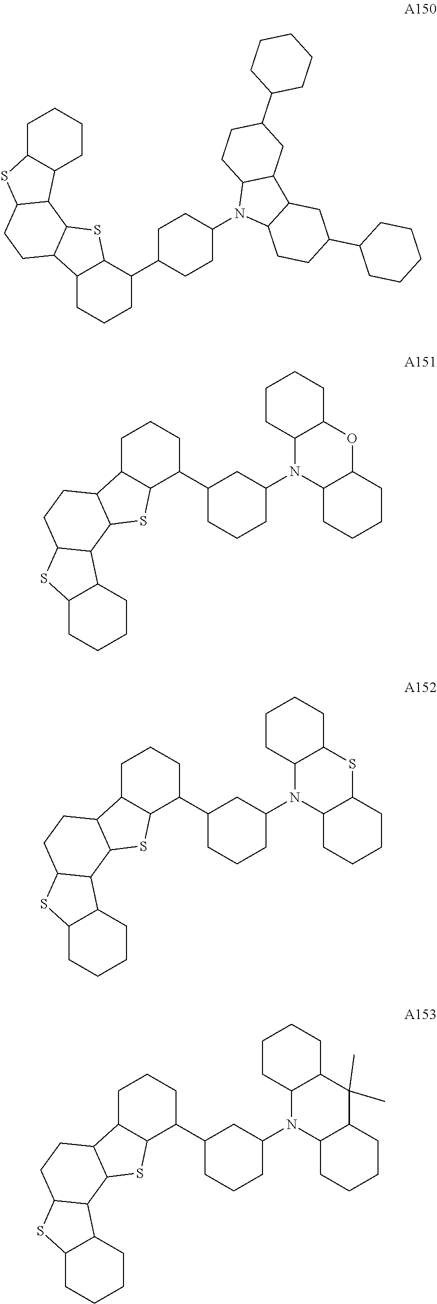 [SMILES: C1CCC(C2CCC3C(C2)C2CC(C4CCCCC4)CCC2N3C2CCC(C3CCCC4C5CCC6SC7CCCCC7C6C5SC34)CC2)CC1 ; C1CCC2C(C1)OC1CCCCC1N2C1CCCC(C2CCCC3C4CCC5SC6CCCCC6C5C4SC23)C1 ; C1CCC2C(C1)SC1CCC3C4CCCC(C5CCCC(N6C7CCCCC7SC7CCCCC76)C5)C4SC3C12 ; CC1(C)C2CCCCC2N(C2CCCC(C3CCCC4C5CCC6SC7CCCCC7C6C5SC34)C2)C2CCCCC21]

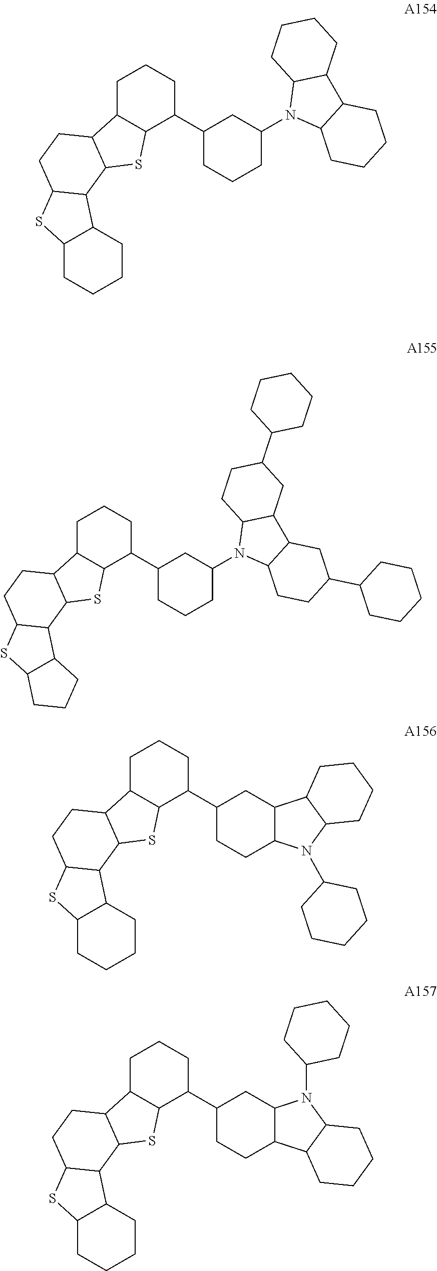 [SMILES: C1CCC2C(C1)SC1CCC3C4CCCC(C5CCCC(N6C7CCCCC7C7CCCCC76)C5)C4SC3C12 ; C1CCC(C2CCC3C(C2)C2CC(C4CCCCC4)CCC2N3C2CCCC(C3CCCC4C5CCC6SC7CCCC7C6C5SC34)C2)CC1 ; C1CCC(N2C3CCCCC3C3CC(C4CCCC5C6CCC7SC8CCCCC8C7C6SC45)CCC32)CC1 ; C1CCC(N2C3CCCCC3C3CCC(C4CCCC5C6CCC7SC8CCCCC8C7C6SC45)CC32)CC1]

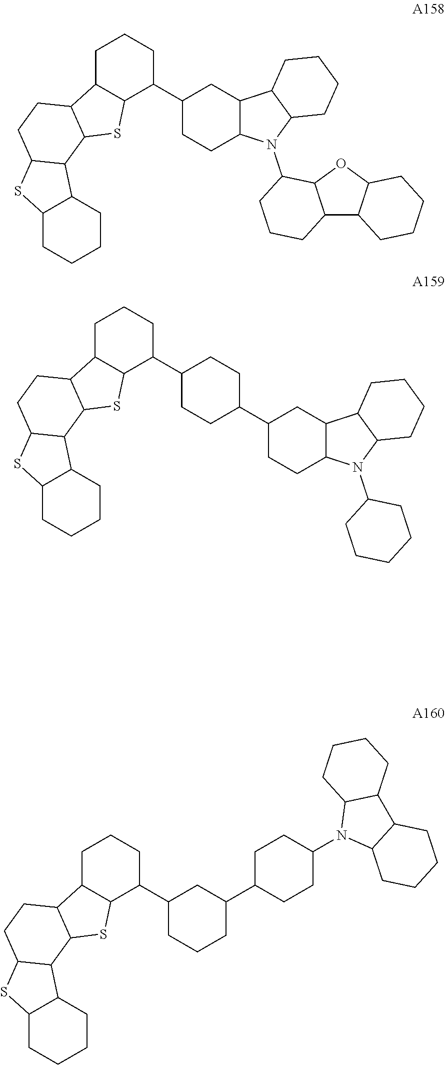 [SMILES: C1CCC2C(C1)OC1C2CCCC1N1C2CCCCC2C2CC(C3CCCC4C5CCC6SC7CCCCC7C6C5SC34)CCC21 ; C1CCC(N2C3CCCCC3C3CC(C4CCC(C5CCCC6C7CCC8SC9CCCCC9C8C7SC56)CC4)CCC32)CC1 ; C1CC(C2CCC(N3C4CCCCC4C4CCCCC43)CC2)CC(C2CCCC3C4CCC5SC6CCCCC6C5C4SC23)C1]

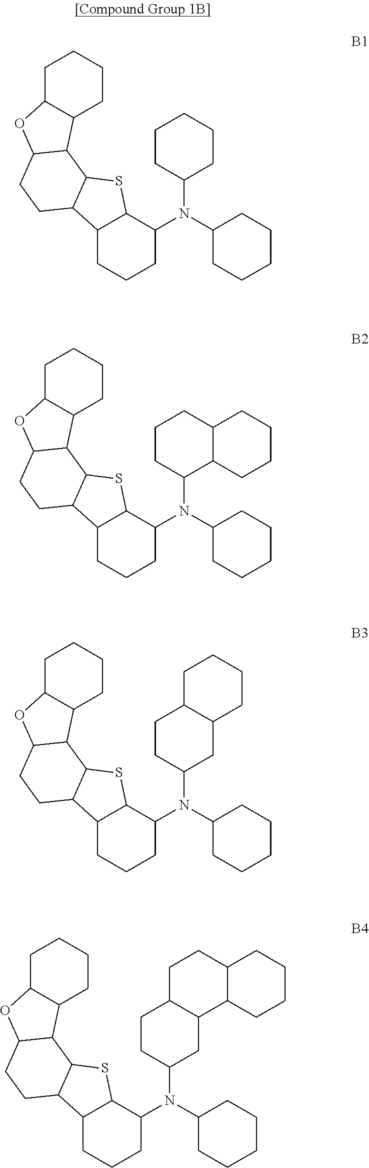 [SMILES: C1CCC(N(C2CCCCC2)C2CCCC3C4CCC5OC6CCCCC6C5C4SC32)CC1 ; C1CCC(N(C2CCCC3CCCCC32)C2CCCC3C4CCC5OC6CCCCC6C5C4SC32)CC1 ; C1CCC(N(C2CCC3CCCCC3C2)C2CCCC3C4CCC5OC6CCCCC6C5C4SC32)CC1 ; C1CCC(N(C2CCC3CCC4CCCCC4C3C2)C2CCCC3C4CCC5OC6CCCCC6C5C4SC32)CC1]

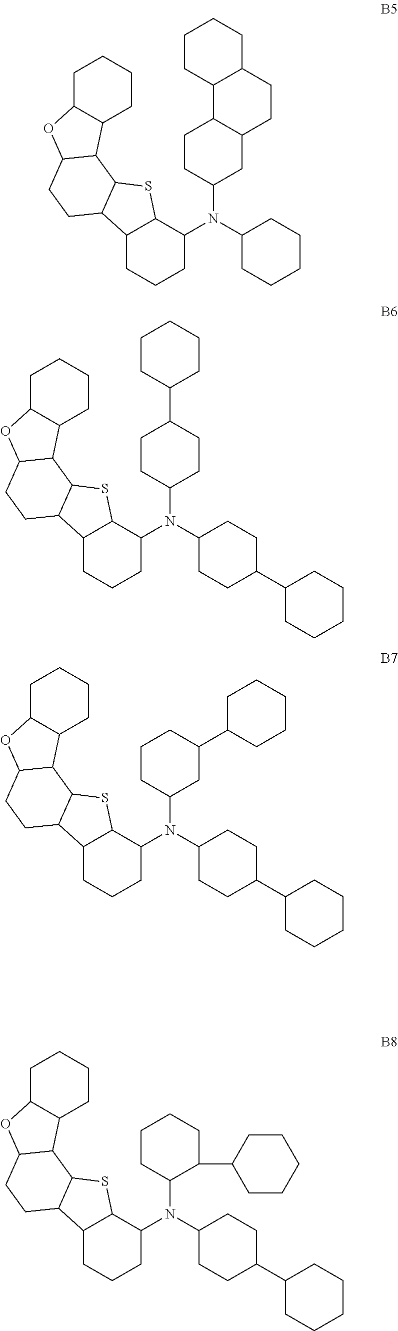

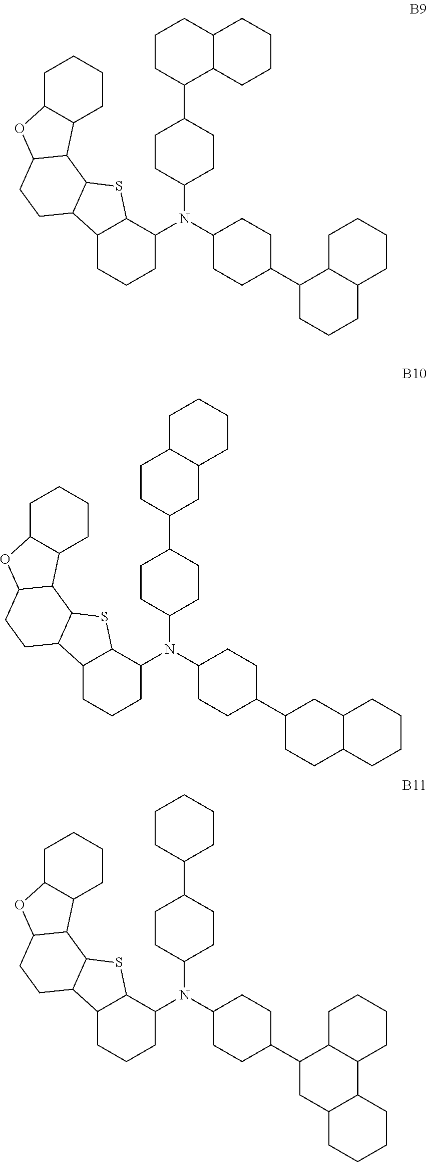 [SMILES: C1CCC2C(C1)CCCC2C1CCC(N(C2CCC(C3CCCC4CCCCC43)CC2)C2CCCC3C4CCC5OC6CCCCC6C5C4SC32)CC1 ; C1CCC2CC(C3CCC(N(C4CCC(C5CCC6CCCCC6C5)CC4)C4CCCC5C6CCC7OC8CCCCC8C7C6SC54)CC3)CCC2C1 ; C1CCC(C2CCC(N(C3CCC(C4CC5CCCCC5C5CCCCC45)CC3)C3CCCC4C5CCC6OC7CCCCC7C6C5SC43)CC2)CC1]

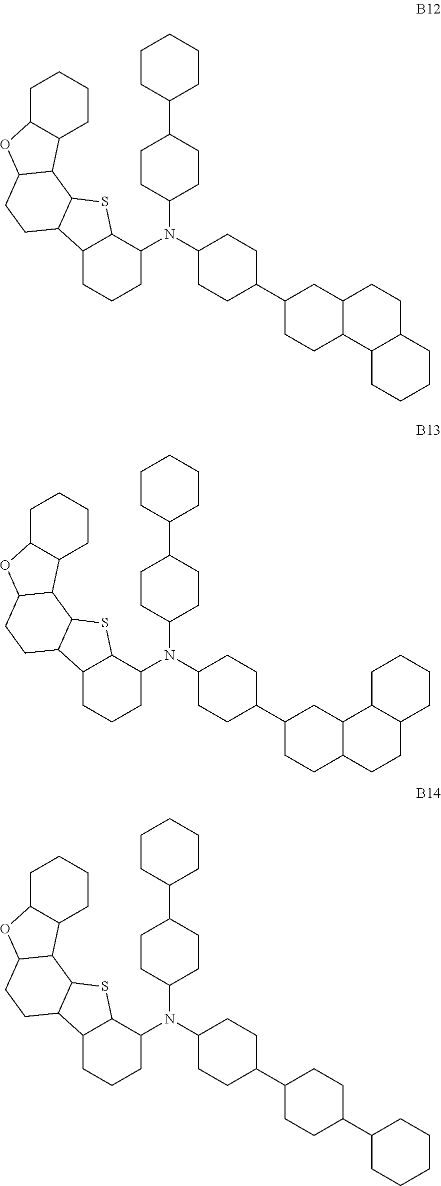 [SMILES: C1CCC(C2CCC(N(C3CCC(C4CCC5C(CCC6CCCCC65)C4)CC3)C3CCCC4C5CCC6OC7CCCCC7C6C5SC43)CC2)CC1 ; C1CCC(C2CCC(N(C3CCC(C4CCC5CCC6CCCCC6C5C4)CC3)C3CCCC4C5CCC6OC7CCCCC7C6C5SC43)CC2)CC1 ; C1CCC(C2CCC(C3CCC(N(C4CCC(C5CCCCC5)CC4)C4CCCC5C6CCC7OC8CCCCC8C7C6SC54)CC3)CC2)CC1]

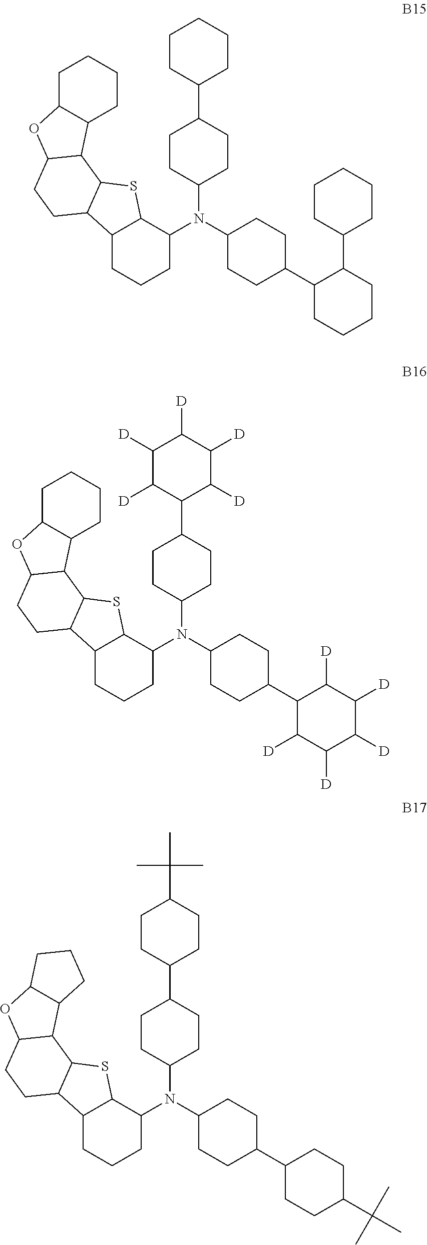 [SMILES: C1CCC(C2CCC(N(C3CCC(C4CCCCC4C4CCCCC4)CC3)C3CCCC4C5CCC6OC7CCCCC7C6C5SC43)CC2)CC1 ; [2H]C1C([2H])C([2H])C(C2CCC(N(C3CCC(C4C([2H])C([2H])C([2H])C([2H])C4[2H])CC3)C3CCCC4C5CCC6OC7CCCCC7C6C5SC43)CC2)C([2H])C1[2H] ; CC(C)(C)C1CCC(C2CCC(N(C3CCC(C4CCC(C(C)(C)C)CC4)CC3)C3CCCC4C5CCC6OC7CCCC7C6C5SC43)CC2)CC1]

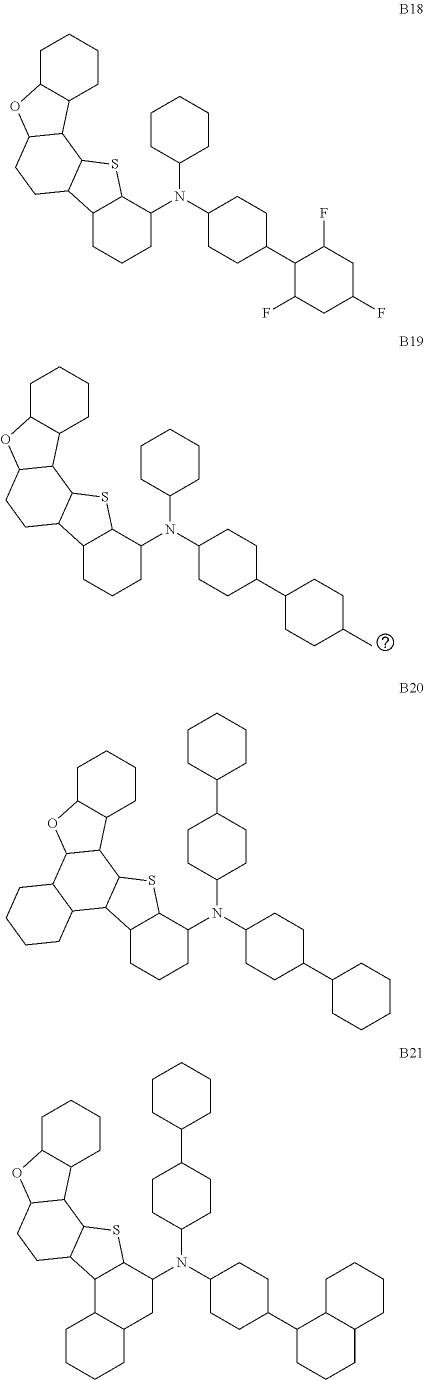 [SMILES: FC1CC(F)C(C2CCC(N(C3CCCCC3)C3CCCC4C5CCC6OC7CCCCC7C6C5SC43)CC2)C(F)C1 ; CC1CCC(C2CCC(N(C3CCCCC3)C3CCCC4C5CCC6OC7CCCCC7C6C5SC43)CC2)CC1 ; C1CCC(C2CCC(N(C3CCC(C4CCCCC4)CC3)C3CCCC4C3SC3C4C4CCCCC4C4OC5CCCCC5C43)CC2)CC1 ; C1CCC(C2CCC(N(C3CCC(C4CCCC5CCCCC54)CC3)C3CC4CCCCC4C4C5CCC6OC7CCCCC7C6C5SC43)CC2)CC1]

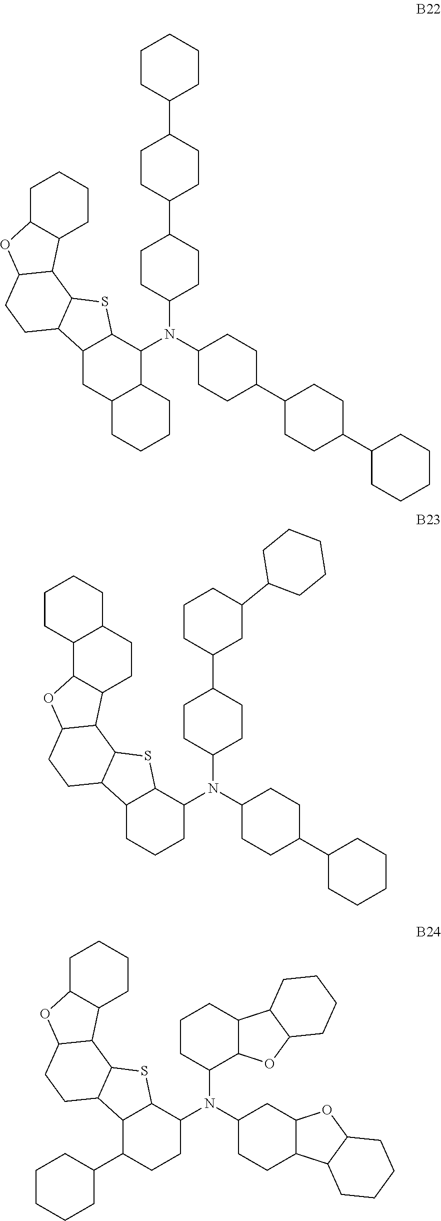 [SMILES: C1CCC(C2CCC(C3CCC(N(C4CCC(C5CCC(C6CCCCC6)CC5)CC4)C4C5CCCCC5CC5C6CCC7OC8CCCCC8C7C6SC54)CC3)CC2)CC1 ; C1CCC(C2CCC(N(C3CCC(C4CCCC(C5CCCCC5)C4)CC3)C3CCCC4C5CCC6OC7C8CCCCC8CCC7C6C5SC43)CC2)CC1 ; C1CCC(C2CCC(N(C3CCC4C(C3)OC3CCCCC34)C3CCCC4C5CCCCC5OC43)C3SC4C(CCC5OC6CCCCC6C54)C23)CC1]

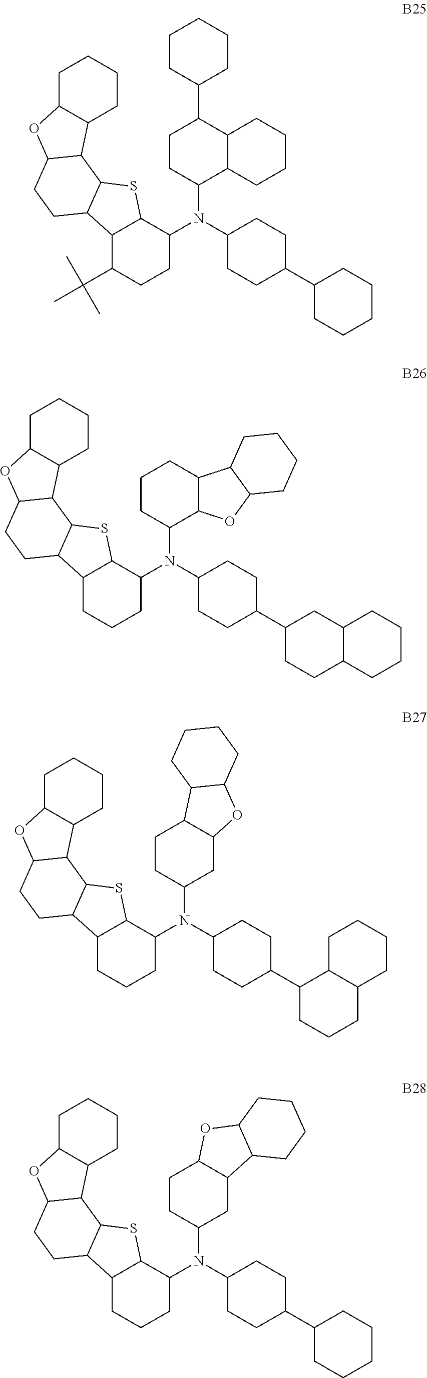 [SMILES: CC(C)(C)C1CCC(N(C2CCC(C3CCCCC3)CC2)C2CCC(C3CCCCC3)C3CCCCC32)C2SC3C(CCC4OC5CCCCC5C43)C21 ; C1CCC2CC(C3CCC(N(C4CCCC5C6CCCCC6OC54)C4CCCC5C6CCC7OC8CCCCC8C7C6SC54)CC3)CCC2C1 ; C1CCC2C(C1)CCCC2C1CCC(N(C2CCC3C(C2)OC2CCCCC23)C2CCCC3C4CCC5OC6CCCCC6C5C4SC32)CC1 ; C1CCC(C2CCC(N(C3CCC4OC5CCCCC5C4C3)C3CCCC4C5CCC6OC7CCCCC7C6C5SC43)CC2)CC1]

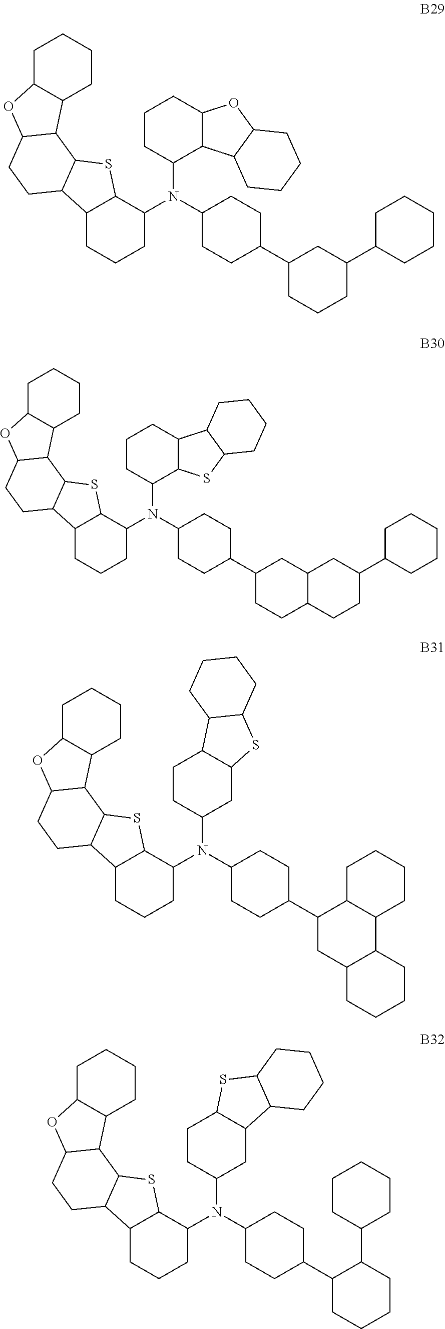 [SMILES: C1CCC(C2CCCC(C3CCC(N(C4CCCC5C6CCC7OC8CCCCC8C7C6SC54)C4CCCC5OC6CCCCC6C54)CC3)C2)CC1 ; C1CCC(C2CCC3CCC(C4CCC(N(C5CCCC6C7CCCCC7SC65)C5CCCC6C7CCC8OC9CCCCC9C8C7SC65)CC4)CC3C2)CC1 ; C1CCC2C(C1)CC(C1CCC(N(C3CCC4C(C3)SC3CCCCC34)C3CCCC4C5CCC6OC7CCCCC7C6C5SC43)CC1)C1CCCCC21 ; C1CCC(C2CCCCC2C2CCC(N(C3CCC4SC5CCCCC5C4C3)C3CCCC4C5CCC6OC7CCCCC7C6C5SC43)CC2)CC1]

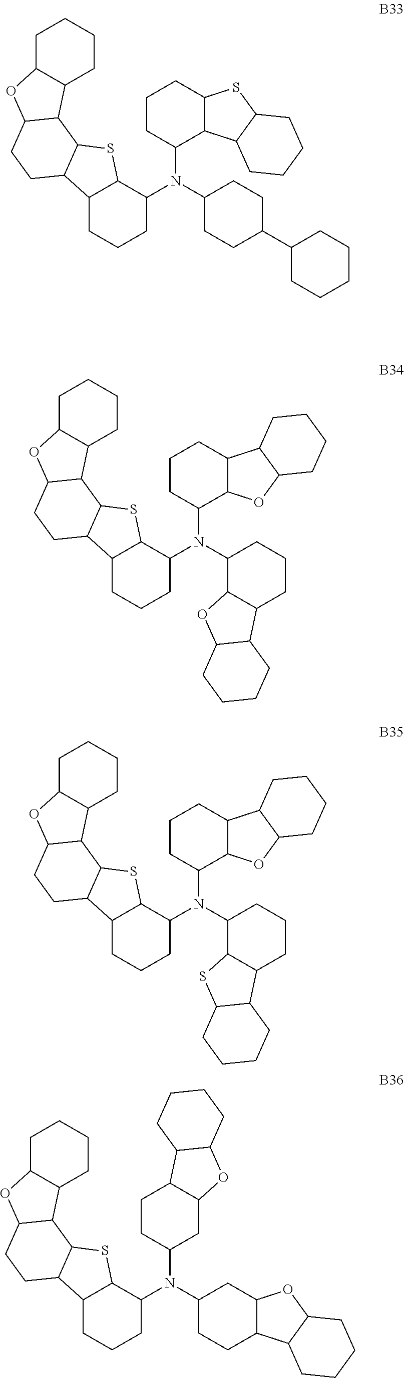

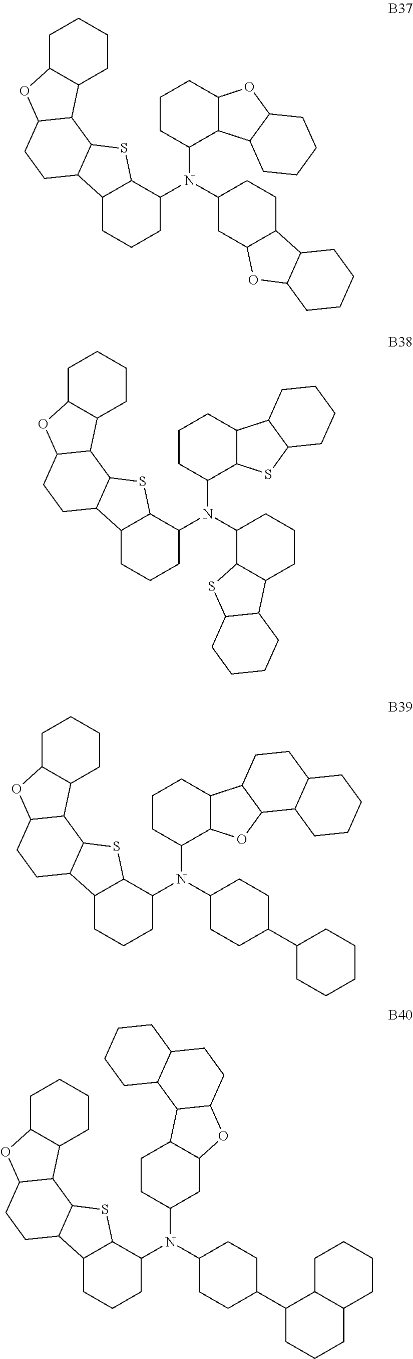 [SMILES: C1CCC2C(C1)OC1CC(N(C3CCCC4C5CCC6OC7CCCCC7C6C5SC43)C3CCCC4OC5CCCCC5C43)CCC12 ; C1CCC2C(C1)OC1CCC3C4CCCC(N(C5CCCC6C7CCCCC7SC65)C5CCCC6C7CCCCC7SC65)C4SC3C12 ; C1CCC(C2CCC(N(C3CCCC4C5CCC6CCCCC6C5OC43)C3CCCC4C5CCC6OC7CCCCC7C6C5SC43)CC2)CC1 ; C1CCC2C(C1)CCCC2C1CCC(N(C2CCC3C(C2)OC2CCC4CCCCC4C23)C2CCCC3C4CCC5OC6CCCCC6C5C4SC32)CC1]

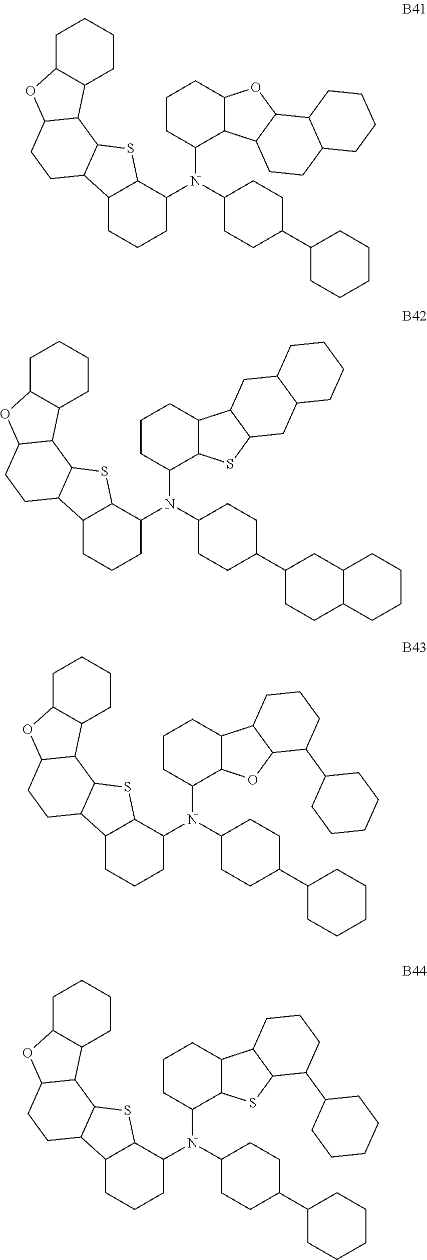 [SMILES: C1CCC(C2CCC(N(C3CCCC4C5CCC6OC7CCCCC7C6C5SC43)C3CCCC4OC5C6CCCCC6CCC5C43)CC2)CC1 ; C1CCC2CC(C3CCC(N(C4CCCC5C6CC7CCCCC7CC6SC54)C4CCCC5C6CCC7OC8CCCCC8C7C6SC54)CC3)CCC2C1 ; C1CCC(C2CCC(N(C3CCCC4C5CCCC(C6CCCCC6)C5OC43)C3CCCC4C5CCC6OC7CCCCC7C6C5SC43)CC2)CC1 ; C1CCC(C2CCC(N(C3CCCC4C5CCCC(C6CCCCC6)C5SC43)C3CCCC4C5CCC6OC7CCCCC7C6C5SC43)CC2)CC1]

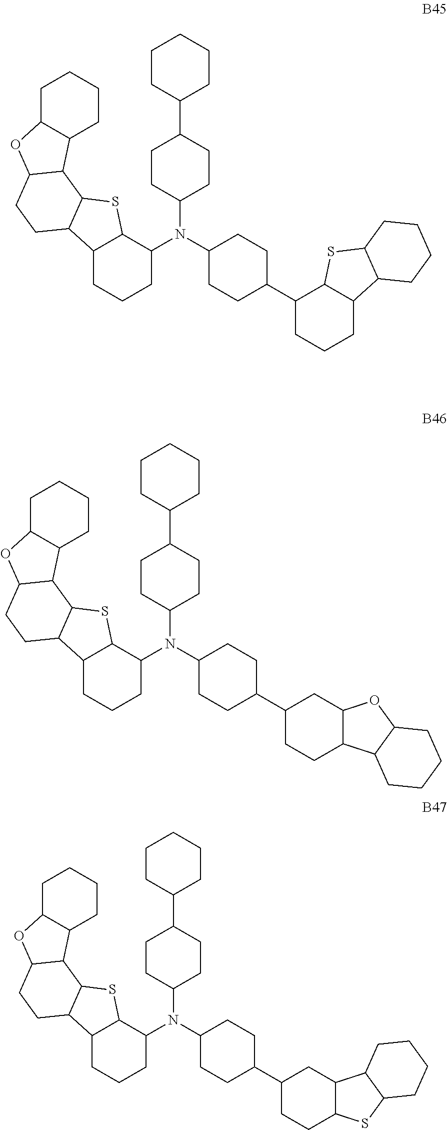 [SMILES: C1CCC(C2CCC(N(C3CCC(C4CCCC5C6CCCCC6SC45)CC3)C3CCCC4C5CCC6OC7CCCCC7C6C5SC43)CC2)CC1 ; C1CCC(C2CCC(N(C3CCC(C4CCC5C(C4)OC4CCCCC45)CC3)C3CCCC4C5CCC6OC7CCCCC7C6C5SC43)CC2)CC1 ; C1CCC(C2CCC(N(C3CCC(C4CCC5SC6CCCCC6C5C4)CC3)C3CCCC4C5CCC6OC7CCCCC7C6C5SC43)CC2)CC1]

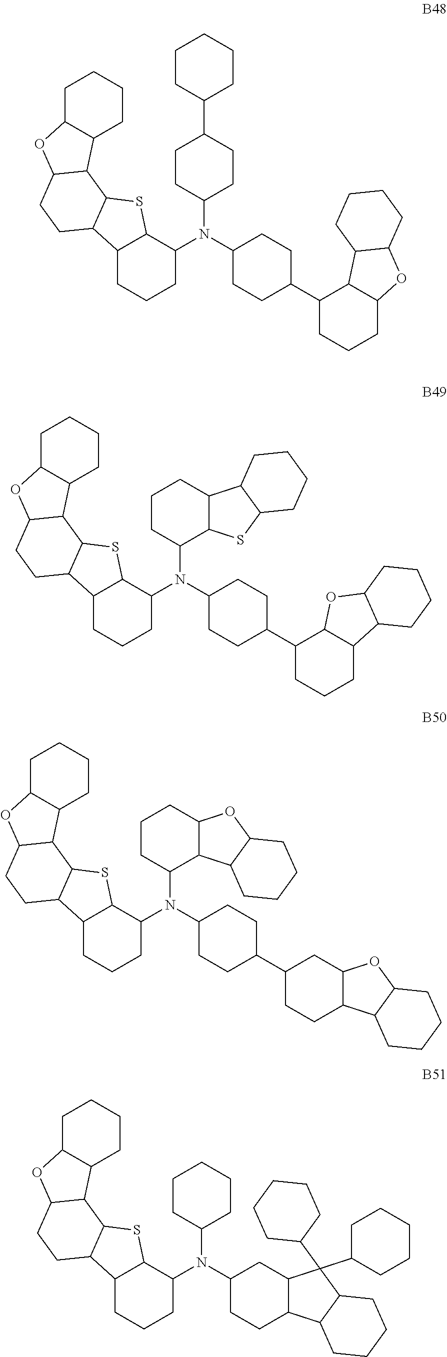 [SMILES: C1CCC(C2CCC(N(C3CCC(C4CCCC5OC6CCCCC6C54)CC3)C3CCCC4C5CCC6OC7CCCCC7C6C5SC43)CC2)CC1 ; C1CCC2C(C1)OC1C(C3CCC(N(C4CCCC5C6CCCCC6SC54)C4CCCC5C6CCC7OC8CCCCC8C7C6SC54)CC3)CCCC21 ; C1CCC2C(C1)OC1CC(C3CCC(N(C4CCCC5C6CCC7OC8CCCCC8C7C6SC54)C4CCCC5OC6CCCCC6C54)CC3)CCC12 ; C1CCC(N(C2CCC3C4CCCCC4C(C4CCCCC4)(C4CCCCC4)C3C2)C2CCCC3C4CCC5OC6CCCCC6C5C4SC32)CC1]

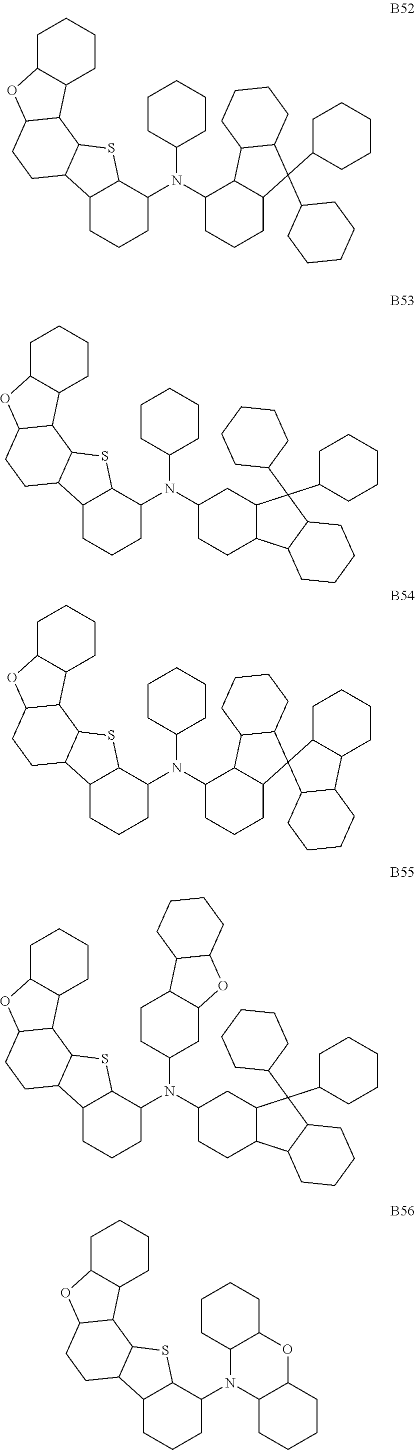 [SMILES: C1CCC(N(C2CCCC3C4CCC5OC6CCCCC6C5C4SC32)C2CCCC3C2C2CCCCC2C3(C2CCCCC2)C2CCCCC2)CC1 ; C1CCC(N(C2CCC3C4CCCCC4C(C4CCCCC4)(C4CCCCC4)C3C2)C2CCCC3C4CCC5OC6CCCCC6C5C4SC32)CC1 ; C1CCC(N(C2CCCC3C4CCC5OC6CCCCC6C5C4SC32)C2CCCC3C2C2CCCCC2C32C3CCCCC3C3CCCCC32)CC1 ; C1CCC(C2(C3CCCCC3)C3CCCCC3C3CCC(N(C4CCC5C(C4)OC4CCCCC45)C4CCCC5C6CCC7OC8CCCCC8C7C6SC54)CC32)CC1 ; C1CCC2C(C1)OC1CCC3C4CCCC(N5C6CCCCC6OC6CCCCC65)C4SC3C12]

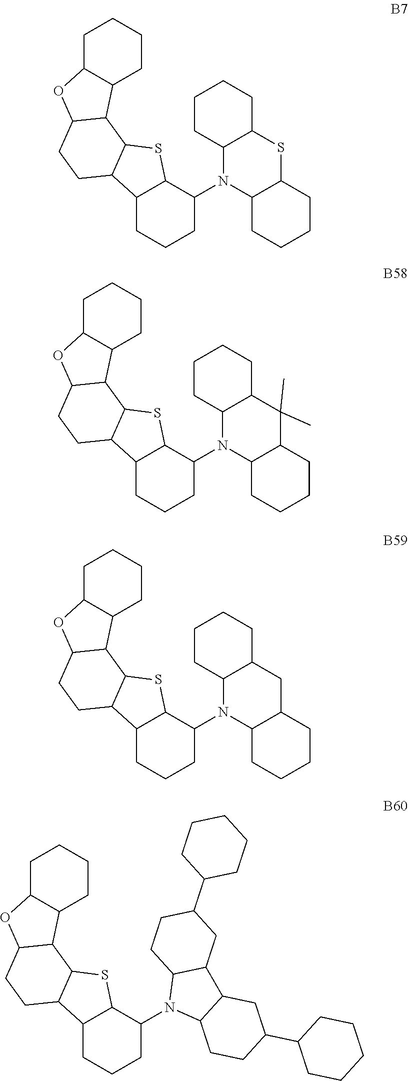 [SMILES: C1CCC2C(C1)OC1CCC3C4CCCC(N5C6CCCCC6SC6CCCCC65)C4SC3C12 ; CC1(C)C2CCCCC2N(C2CCCC3C4CCC5OC6CCCCC6C5C4SC32)C2CCCCC21 ; C1CCC2C(C1)CC1CCCCC1N2C1CCCC2C3CCC4OC5CCCCC5C4C3SC21 ; C1CCC(C2CCC3C(C2)C2CC(C4CCCCC4)CCC2N3C2CCCC3C4CCC5OC6CCCCC6C5C4SC32)CC1]

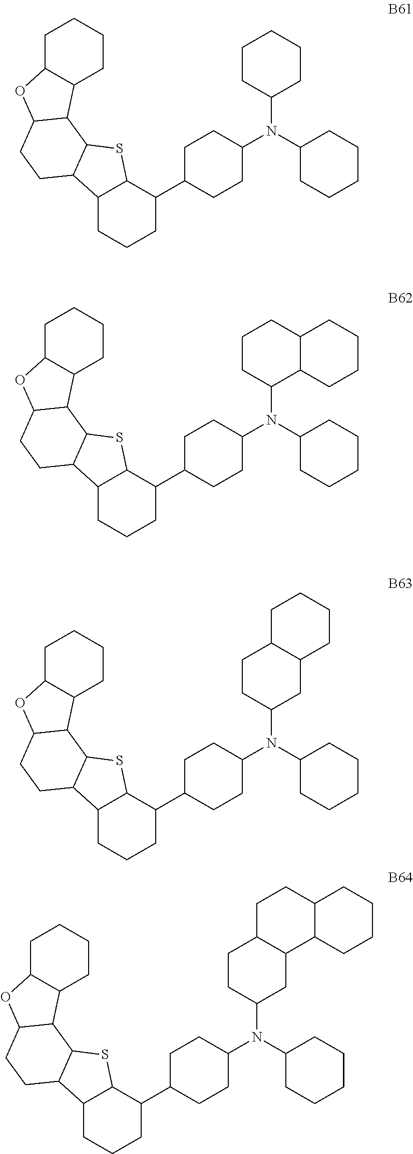 [SMILES: C1CCC(N(C2CCCCC2)C2CCC(C3CCCC4C5CCC6OC7CCCCC7C6C5SC34)CC2)CC1 ; C1CCC(N(C2CCC(C3CCCC4C5CCC6OC7CCCCC7C6C5SC34)CC2)C2CCCC3CCCCC32)CC1 ; C1CCC(N(C2CCC(C3CCCC4C5CCC6OC7CCCCC7C6C5SC34)CC2)C2CCC3CCCCC3C2)CC1 ; C1CCC(N(C2CCC(C3CCCC4C5CCC6OC7CCCCC7C6C5SC34)CC2)C2CCC3CCC4CCCCC4C3C2)CC1]

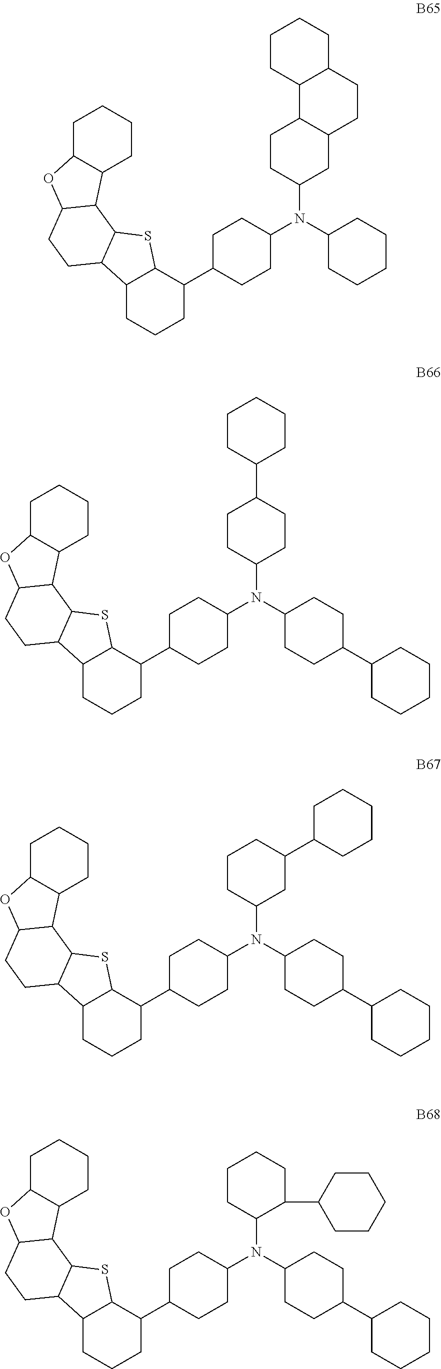 [SMILES: C1CCC(N(C2CCC(C3CCCC4C5CCC6OC7CCCCC7C6C5SC34)CC2)C2CCC3C(CCC4CCCCC43)C2)CC1 ; C1CCC(C2CCC(N(C3CCC(C4CCCCC4)CC3)C3CCC(C4CCCC5C6CCC7OC8CCCCC8C7C6SC45)CC3)CC2)CC1 ; C1CCC(C2CCC(N(C3CCC(C4CCCC5C6CCC7OC8CCCCC8C7C6SC45)CC3)C3CCCC(C4CCCCC4)C3)CC2)CC1 ; C1CCC(C2CCC(N(C3CCC(C4CCCC5C6CCC7OC8CCCCC8C7C6SC45)CC3)C3CCCCC3C3CCCCC3)CC2)CC1]

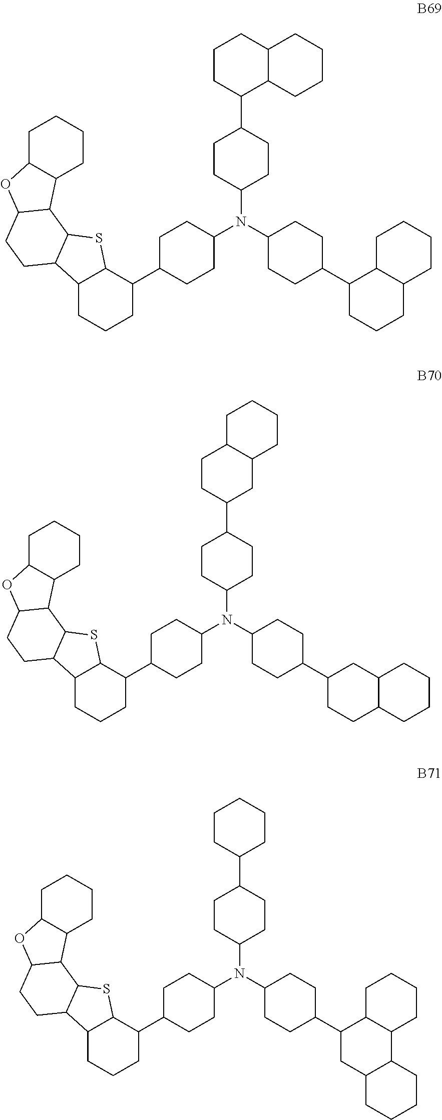 [SMILES: C1CCC2C(C1)CCCC2C1CCC(N(C2CCC(C3CCCC4CCCCC43)CC2)C2CCC(C3CCCC4C5CCC6OC7CCCCC7C6C5SC34)CC2)CC1 ; C1CCC2CC(C3CCC(N(C4CCC(C5CCC6CCCCC6C5)CC4)C4CCC(C5CCCC6C7CCC8OC9CCCCC9C8C7SC56)CC4)CC3)CCC2C1 ; C1CCC(C2CCC(N(C3CCC(C4CC5CCCCC5C5CCCCC45)CC3)C3CCC(C4CCCC5C6CCC7OC8CCCCC8C7C6SC45)CC3)CC2)CC1]

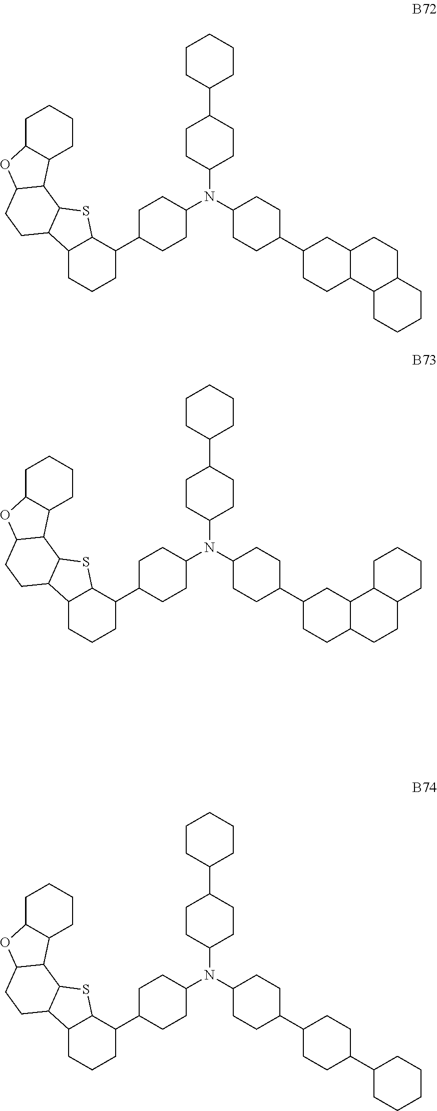 [SMILES: C1CCC(C2CCC(N(C3CCC(C4CCC5C(CCC6CCCCC65)C4)CC3)C3CCC(C4CCCC5C6CCC7OC8CCCCC8C7C6SC45)CC3)CC2)CC1 ; C1CCC(C2CCC(N(C3CCC(C4CCC5CCC6CCCCC6C5C4)CC3)C3CCC(C4CCCC5C6CCC7OC8CCCCC8C7C6SC45)CC3)CC2)CC1 ; C1CCC(C2CCC(C3CCC(N(C4CCC(C5CCCCC5)CC4)C4CCC(C5CCCC6C7CCC8OC9CCCCC9C8C7SC56)CC4)CC3)CC2)CC1]

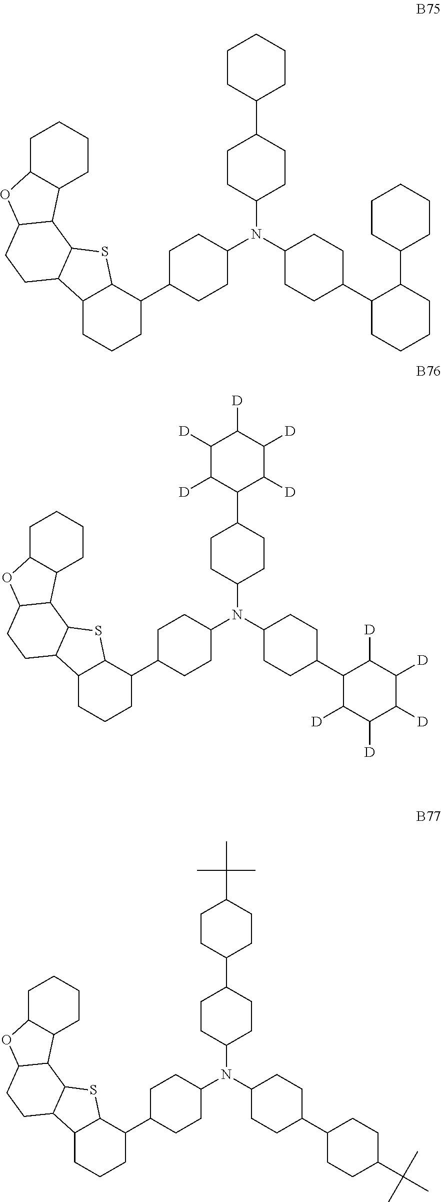 [SMILES: C1CCC(C2CCC(N(C3CCC(C4CCCCC4C4CCCCC4)CC3)C3CCC(C4CCCC5C6CCC7OC8CCCCC8C7C6SC45)CC3)CC2)CC1 ; [2H]C1C([2H])C([2H])C(C2CCC(N(C3CCC(C4CCCC5C6CCC7OC8CCCCC8C7C6SC45)CC3)C3CCC(C4C([2H])C([2H])C([2H])C([2H])C4[2H])CC3)CC2)C([2H])C1[2H] ; CC(C)(C)C1CCC(C2CCC(N(C3CCC(C4CCC(C(C)(C)C)CC4)CC3)C3CCC(C4CCCC5C6CCC7OC8CCCCC8C7C6SC45)CC3)CC2)CC1]

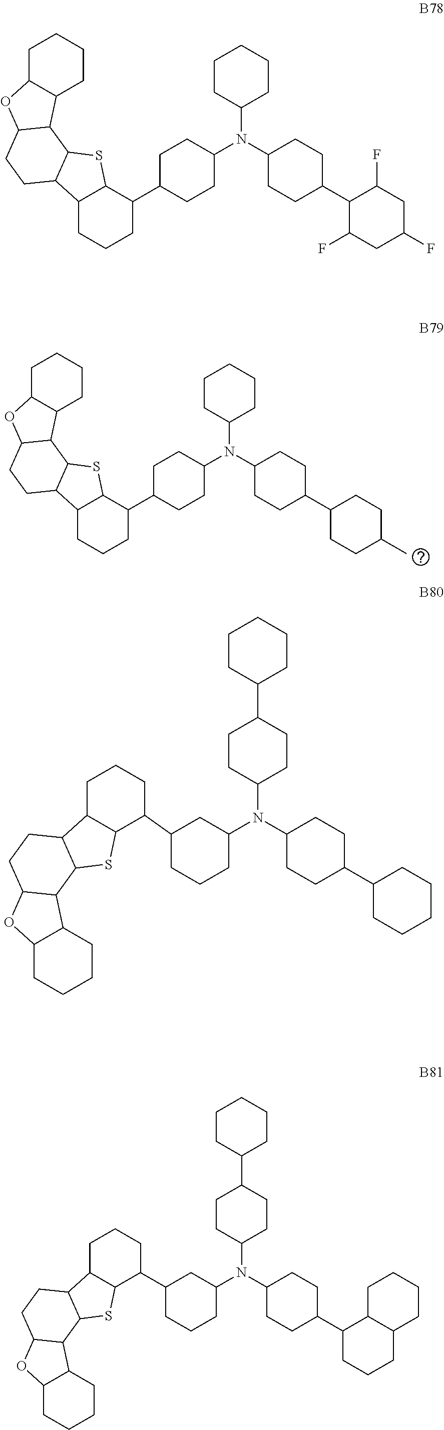 [SMILES: FC1CC(F)C(C2CCC(N(C3CCCCC3)C3CCC(C4CCCC5C6CCC7OC8CCCCC8C7C6SC45)CC3)CC2)C(F)C1 ; CC1CCC(C2CCC(N(C3CCCCC3)C3CCC(C4CCCC5C6CCC7OC8CCCCC8C7C6SC45)CC3)CC2)CC1 ; C1CCC(C2CCC(N(C3CCC(C4CCCCC4)CC3)C3CCCC(C4CCCC5C6CCC7OC8CCCCC8C7C6SC45)C3)CC2)CC1 ; C1CCC(C2CCC(N(C3CCC(C4CCCC5CCCCC54)CC3)C3CCCC(C4CCCC5C6CCC7OC8CCCCC8C7C6SC45)C3)CC2)CC1]

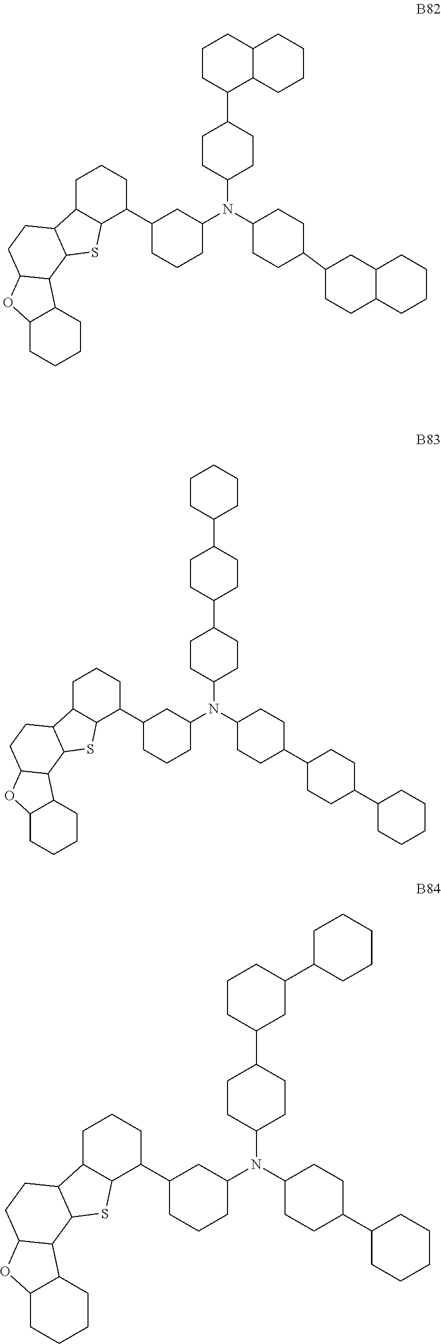 [SMILES: C1CCC2CC(C3CCC(N(C4CCC(C5CCCC6CCCCC65)CC4)C4CCCC(C5CCCC6C7CCC8OC9CCCCC9C8C7SC56)C4)CC3)CCC2C1 ; C1CCC(C2CCC(C3CCC(N(C4CCC(C5CCC(C6CCCCC6)CC5)CC4)C4CCCC(C5CCCC6C7CCC8OC9CCCCC9C8C7SC56)C4)CC3)CC2)CC1 ; C1CCC(C2CCC(N(C3CCC(C4CCCC(C5CCCCC5)C4)CC3)C3CCCC(C4CCCC5C6CCC7OC8CCCCC8C7C6SC45)C3)CC2)CC1]

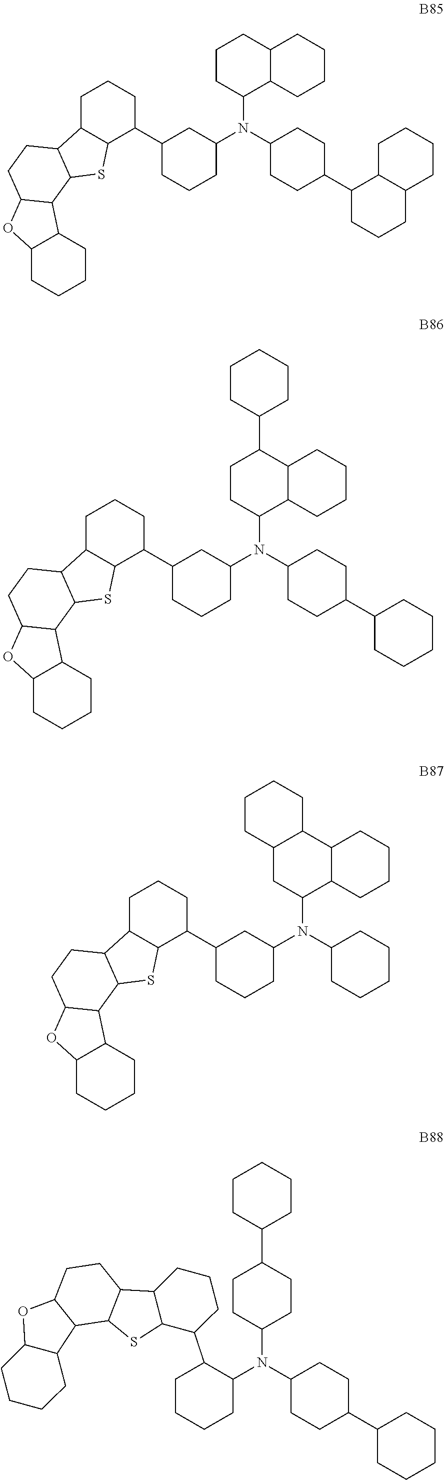 [SMILES: C1CCC2C(C1)CCCC2C1CCC(N(C2CCCC(C3CCCC4C5CCC6OC7CCCCC7C6C5SC34)C2)C2CCCC3CCCCC32)CC1 ; C1CCC(C2CCC(N(C3CCCC(C4CCCC5C6CCC7OC8CCCCC8C7C6SC45)C3)C3CCC(C4CCCCC4)C4CCCCC43)CC2)CC1 ; C1CCC(N(C2CCCC(C3CCCC4C5CCC6OC7CCCCC7C6C5SC34)C2)C2CC3CCCCC3C3CCCCC32)CC1 ; C1CCC(C2CCC(N(C3CCC(C4CCCCC4)CC3)C3CCCCC3C3CCCC4C5CCC6OC7CCCCC7C6C5SC43)CC2)CC1]

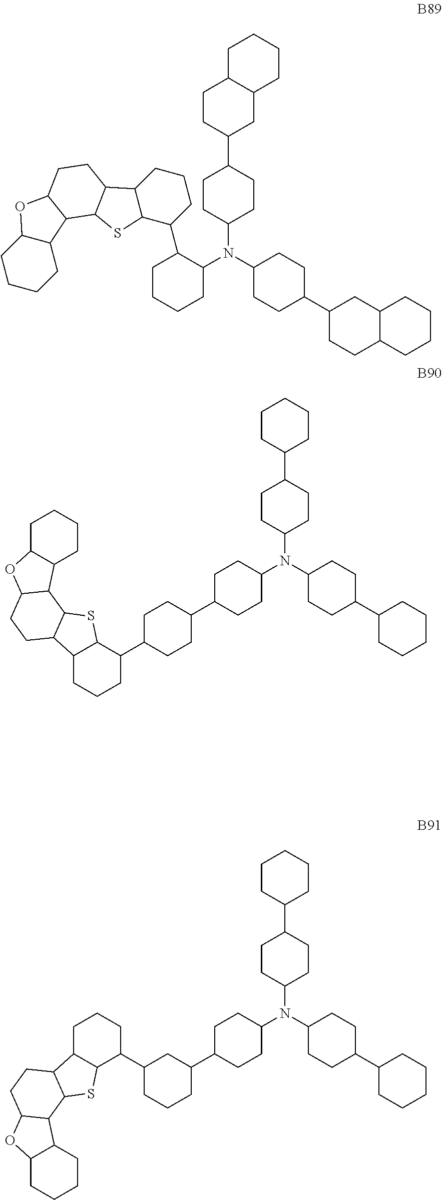 [SMILES: C1CCC2CC(C3CCC(N(C4CCC(C5CCC6CCCCC6C5)CC4)C4CCCCC4C4CCCC5C6CCC7OC8CCCCC8C7C6SC54)CC3)CCC2C1 ; C1CCC(C2CCC(N(C3CCC(C4CCCCC4)CC3)C3CCC(C4CCC(C5CCCC6C7CCC8OC9CCCCC9C8C7SC56)CC4)CC3)CC2)CC1 ; C1CCC(C2CCC(N(C3CCC(C4CCCCC4)CC3)C3CCC(C4CCCC(C5CCCC6C7CCC8OC9CCCCC9C8C7SC56)C4)CC3)CC2)CC1]

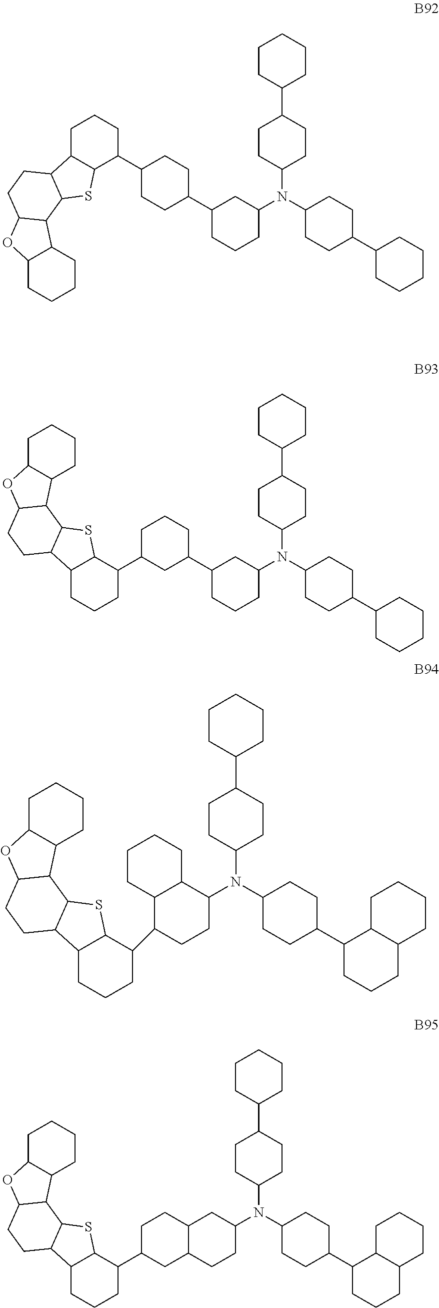 [SMILES: C1CCC(C2CCC(N(C3CCC(C4CCCCC4)CC3)C3CCCC(C4CCC(C5CCCC6C7CCC8OC9CCCCC9C8C7SC56)CC4)C3)CC2)CC1 ; C1CCC(C2CCC(N(C3CCC(C4CCCCC4)CC3)C3CCCC(C4CCCC(C5CCCC6C7CCC8OC9CCCCC9C8C7SC56)C4)C3)CC2)CC1 ; C1CCC(C2CCC(N(C3CCC(C4CCCC5CCCCC54)CC3)C3CCC(C4CCCC5C6CCC7OC8CCCCC8C7C6SC45)C4CCCCC43)CC2)CC1 ; C1CCC(C2CCC(N(C3CCC(C4CCCC5CCCCC54)CC3)C3CCC4CC(C5CCCC6C7CCC8OC9CCCCC9C8C7SC56)CCC4C3)CC2)CC1]

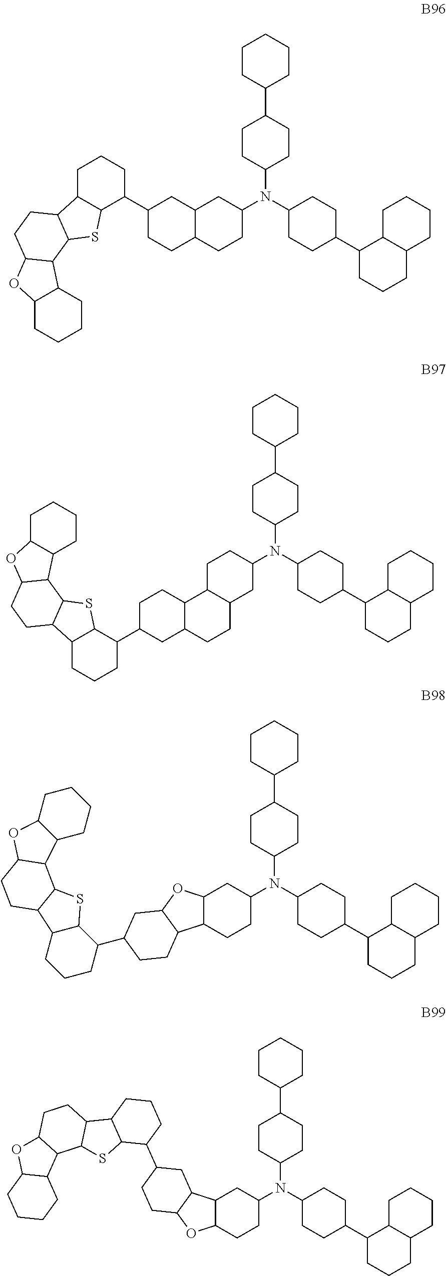 [SMILES: C1CCC(C2CCC(N(C3CCC(C4CCCC5CCCCC54)CC3)C3CCC4CCC(C5CCCC6C7CCC8OC9CCCCC9C8C7SC56)CC4C3)CC2)CC1 ; C1CCC(C2CCC(N(C3CCC(C4CCCC5CCCCC54)CC3)C3CCC4C(CCC5CC(C6CCCC7C8CCC9OC%10CCCCC%10C9C8SC67)CCC54)C3)CC2)CC1 ; C1CCC(C2CCC(N(C3CCC(C4CCCC5CCCCC54)CC3)C3CCC4C(C3)OC3CC(C5CCCC6C7CCC8OC9CCCCC9C8C7SC56)CCC34)CC2)CC1 ; C1CCC(C2CCC(N(C3CCC(C4CCCC5CCCCC54)CC3)C3CCC4OC5CCC(C6CCCC7C8CCC9OC%10CCCCC%10C9C8SC67)CC5C4C3)CC2)CC1]

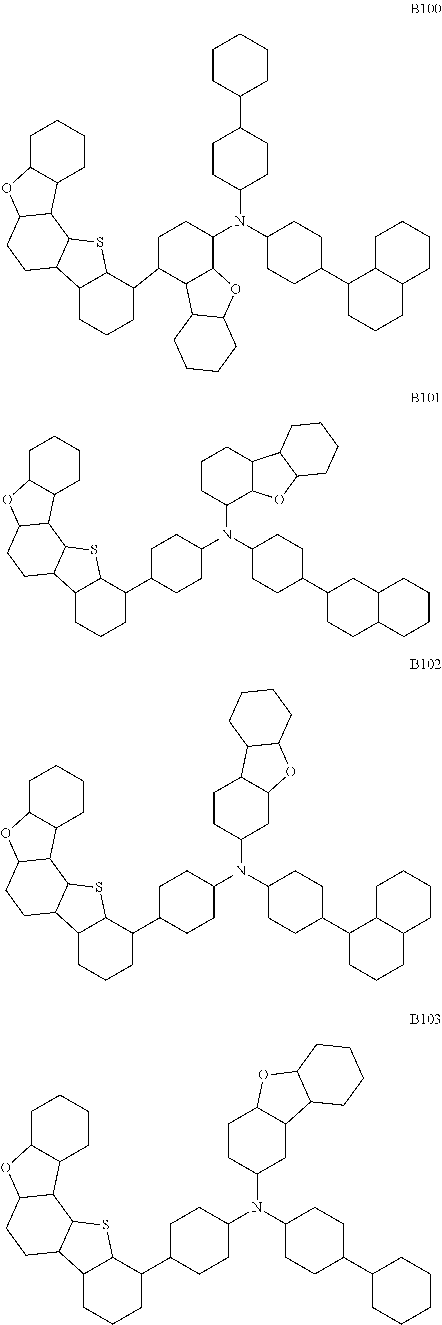 [SMILES: C1CCC(C2CCC(N(C3CCC(C4CCCC5CCCCC54)CC3)C3CCC(C4CCCC5C6CCC7OC8CCCCC8C7C6SC54)C4C5CCCCC5OC43)CC2)CC1 ; C1CCC2CC(C3CCC(N(C4CCC(C5CCCC6C7CCC8OC9CCCCC9C8C7SC56)CC4)C4CCCC5C6CCCCC6OC54)CC3)CCC2C1 ; C1CCC2C(C1)CCCC2C1CCC(N(C2CCC(C3CCCC4C5CCC6OC7CCCCC7C6C5SC34)CC2)C2CCC3C(C2)OC2CCCCC23)CC1 ; C1CCC(C2CCC(N(C3CCC(C4CCCC5C6CCC7OC8CCCCC8C7C6SC45)CC3)C3CCC4OC5CCCCC5C4C3)CC2)CC1]

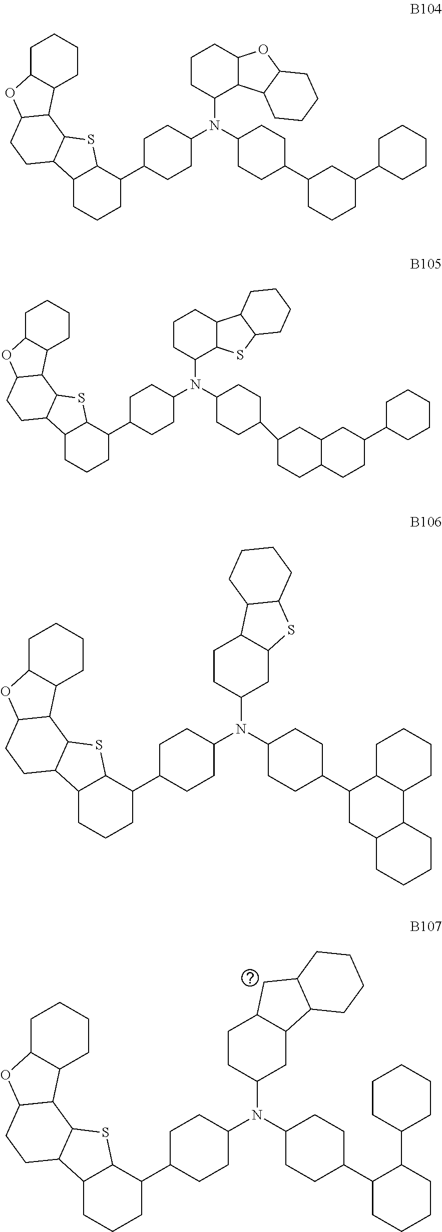 [SMILES: C1CCC(C2CCCC(C3CCC(N(C4CCC(C5CCCC6C7CCC8OC9CCCCC9C8C7SC56)CC4)C4CCCC5OC6CCCCC6C54)CC3)C2)CC1 ; C1CCC(C2CCC3CCC(C4CCC(N(C5CCC(C6CCCC7C8CCC9OC%10CCCCC%10C9C8SC67)CC5)C5CCCC6C7CCCCC7SC65)CC4)CC3C2)CC1 ; C1CCC2C(C1)CC(C1CCC(N(C3CCC(C4CCCC5C6CCC7OC8CCCCC8C7C6SC45)CC3)C3CCC4C(C3)SC3CCCCC34)CC1)C1CCCCC21 ; C1CCC(C2CCCCC2C2CCC(N(C3CCC(C4CCCC5C6CCC7OC8CCCCC8C7C6SC45)CC3)C3CCC4CC5CCCCC5C4C3)CC2)CC1]

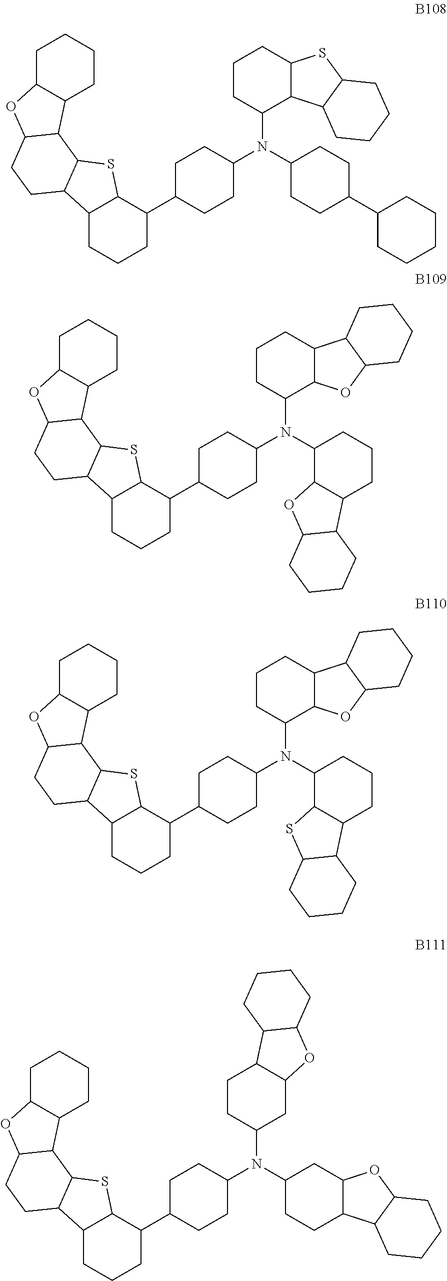

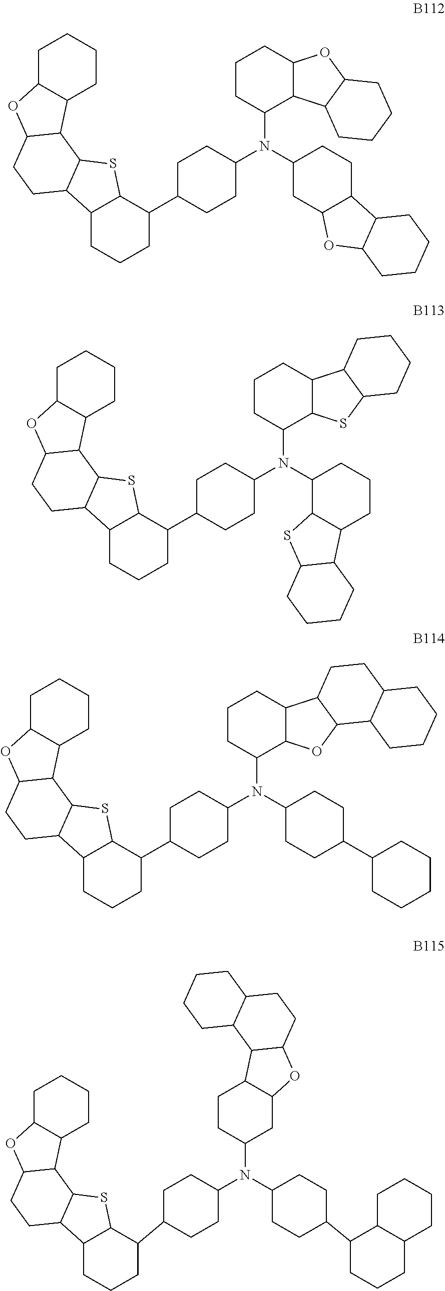 [SMILES: C1CCC2C(C1)OC1CC(N(C3CCC(C4CCCC5C6CCC7OC8CCCCC8C7C6SC45)CC3)C3CCCC4OC5CCCCC5C43)CCC12 ; C1CCC2C(C1)OC1CCC3C4CCCC(C5CCC(N(C6CCCC7C8CCCCC8SC76)C6CCCC7C8CCCCC8SC76)CC5)C4SC3C12 ; C1CCC(C2CCC(N(C3CCC(C4CCCC5C6CCC7OC8CCCCC8C7C6SC45)CC3)C3CCCC4C5CCC6CCCCC6C5OC43)CC2)CC1 ; C1CCC2C(C1)CCCC2C1CCC(N(C2CCC(C3CCCC4C5CCC6OC7CCCCC7C6C5SC34)CC2)C2CCC3C(C2)OC2CCC4CCCCC4C23)CC1]

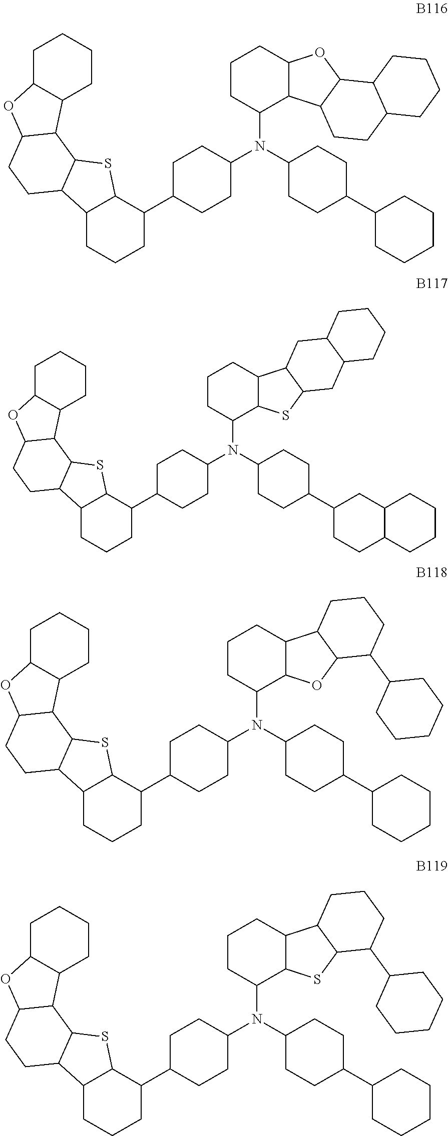

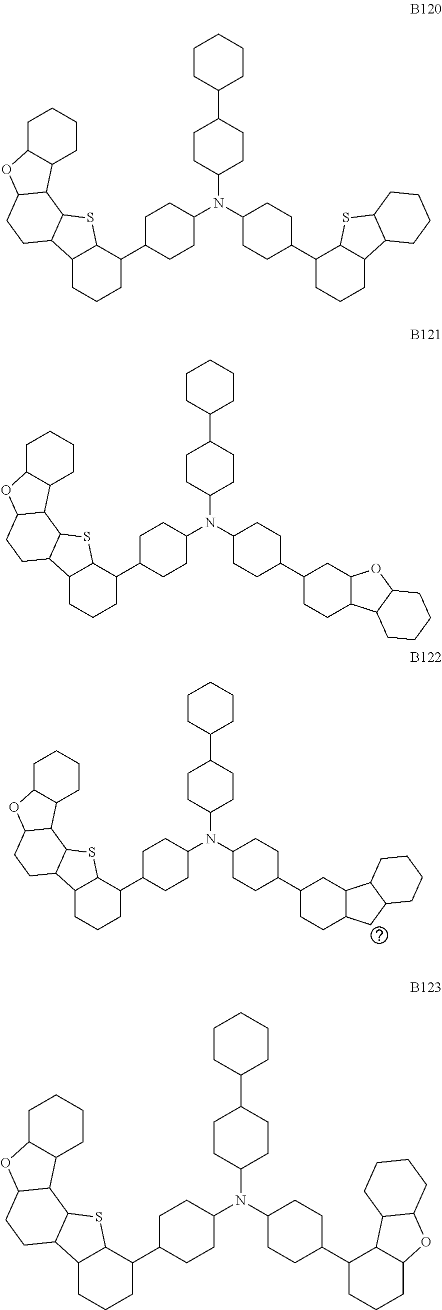 [SMILES: C1CCC(C2CCC(N(C3CCC(C4CCCC5C6CCCCC6SC45)CC3)C3CCC(C4CCCC5C6CCC7OC8CCCCC8C7C6SC45)CC3)CC2)CC1 ; C1CCC(C2CCC(N(C3CCC(C4CCC5C(C4)OC4CCCCC45)CC3)C3CCC(C4CCCC5C6CCC7OC8CCCCC8C7C6SC45)CC3)CC2)CC1 ; C1CCC(C2CCC(N(C3CCC(C4CCC5CC6CCCCC6C5C4)CC3)C3CCC(C4CCCC5C6CCC7OC8CCCCC8C7C6SC45)CC3)CC2)CC1 ; C1CCC(C2CCC(N(C3CCC(C4CCCC5C6CCC7OC8CCCCC8C7C6SC45)CC3)C3CCC(C4CCCC5OC6CCCCC6C54)CC3)CC2)CC1]

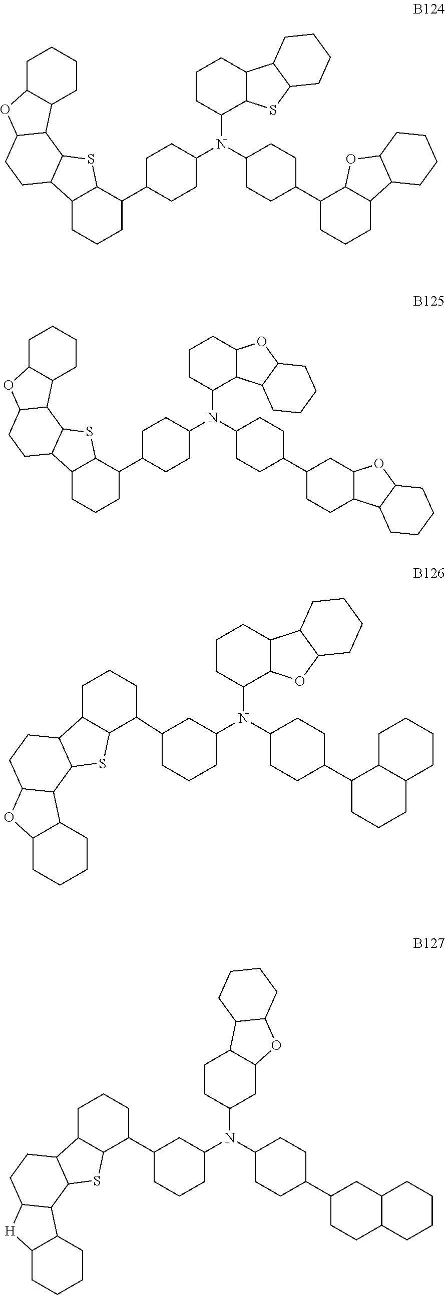

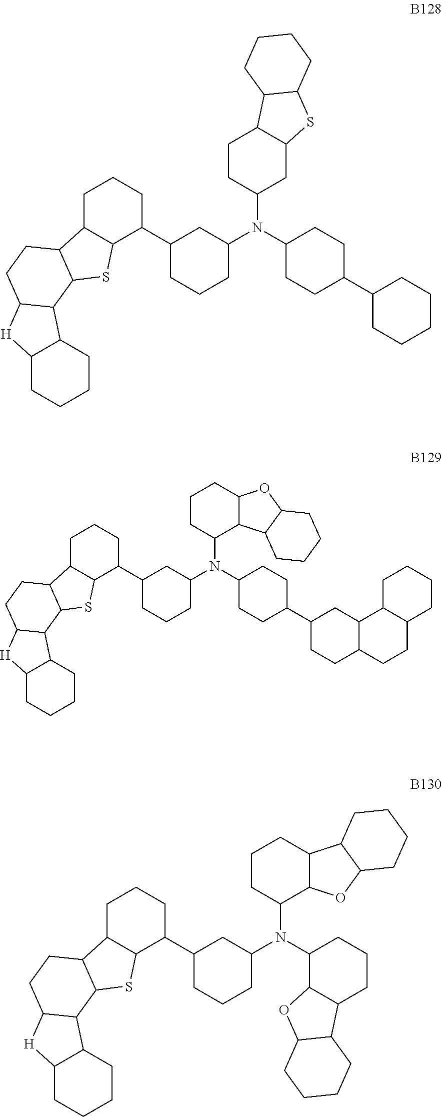

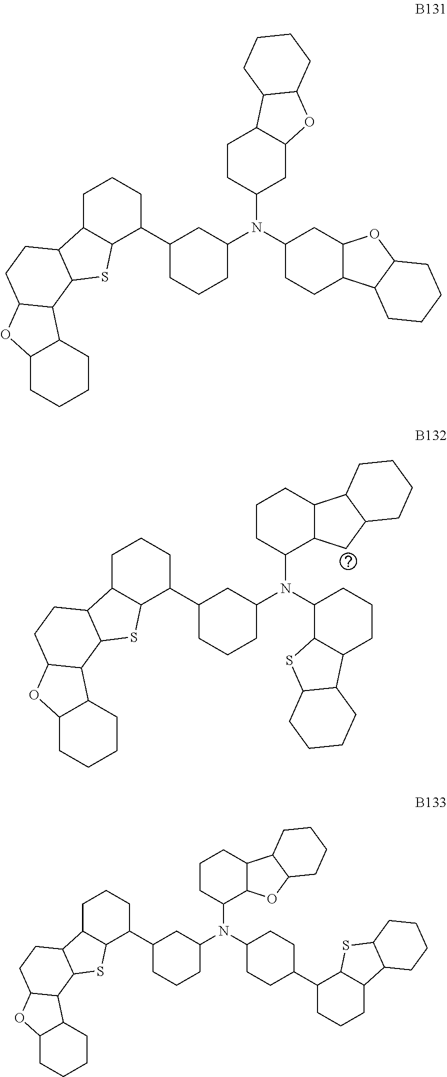 [SMILES: C1CCC2C(C1)OC1CC(N(C3CCCC(C4CCCC5C6CCC7OC8CCCCC8C7C6SC45)C3)C3CCC4C(C3)OC3CCCCC34)CCC12 ; C1CCC2C(C1)CC1C2CCCC1N(C1CCCC(C2CCCC3C4CCC5OC6CCCCC6C5C4SC23)C1)C1CCCC2C3CCCCC3SC21 ; C1CCC2C(C1)OC1C2CCCC1N(C1CCC(C2CCCC3C4CCCCC4SC23)CC1)C1CCCC(C2CCCC3C4CCC5OC6CCCCC6C5C4SC23)C1]

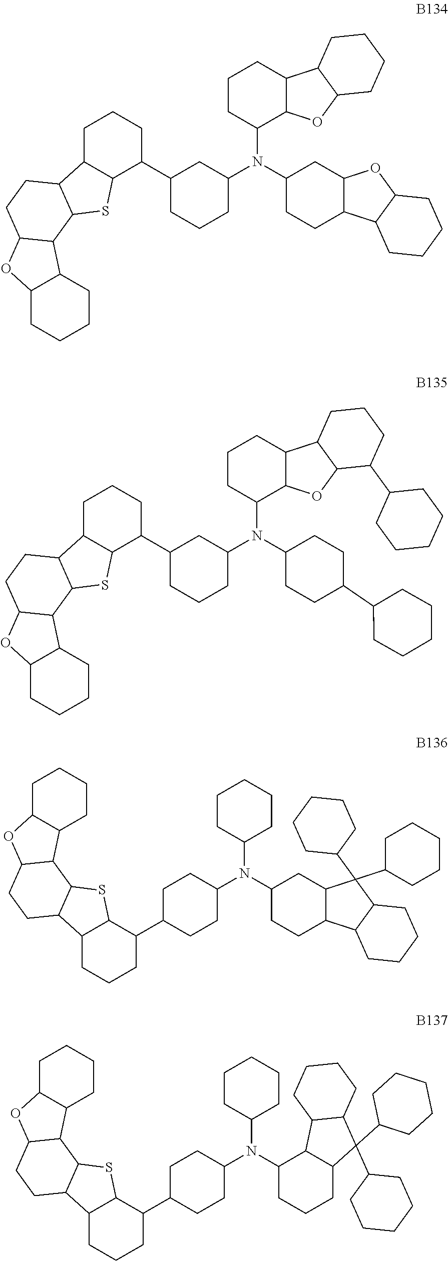 [SMILES: C1CCC2C(C1)OC1CC(N(C3CCCC(C4CCCC5C6CCC7OC8CCCCC8C7C6SC45)C3)C3CCCC4C5CCCCC5OC43)CCC12 ; C1CCC(C2CCC(N(C3CCCC(C4CCCC5C6CCC7OC8CCCCC8C7C6SC45)C3)C3CCCC4C5CCCC(C6CCCCC6)C5OC43)CC2)CC1 ; C1CCC(N(C2CCC(C3CCCC4C5CCC6OC7CCCCC7C6C5SC34)CC2)C2CCC3C4CCCCC4C(C4CCCCC4)(C4CCCCC4)C3C2)CC1 ; C1CCC(N(C2CCC(C3CCCC4C5CCC6OC7CCCCC7C6C5SC34)CC2)C2CCCC3C2C2CCCCC2C3(C2CCCCC2)C2CCCCC2)CC1]

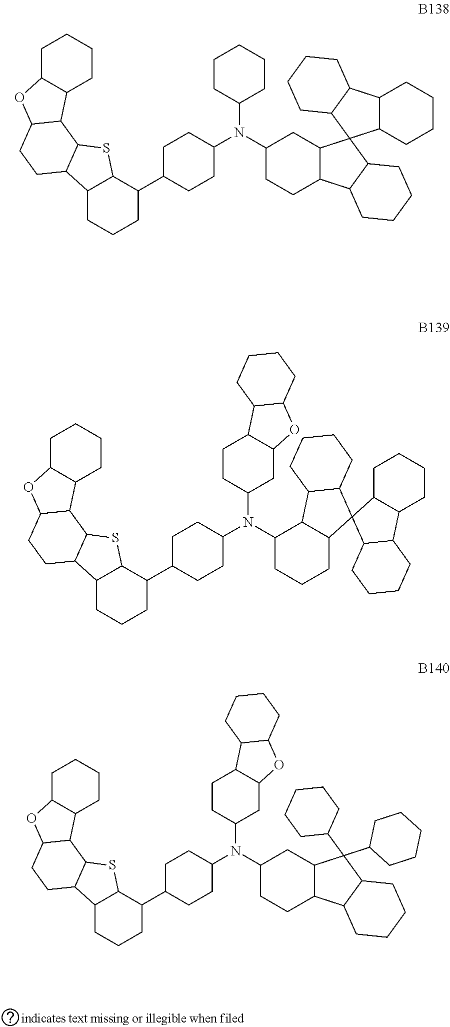

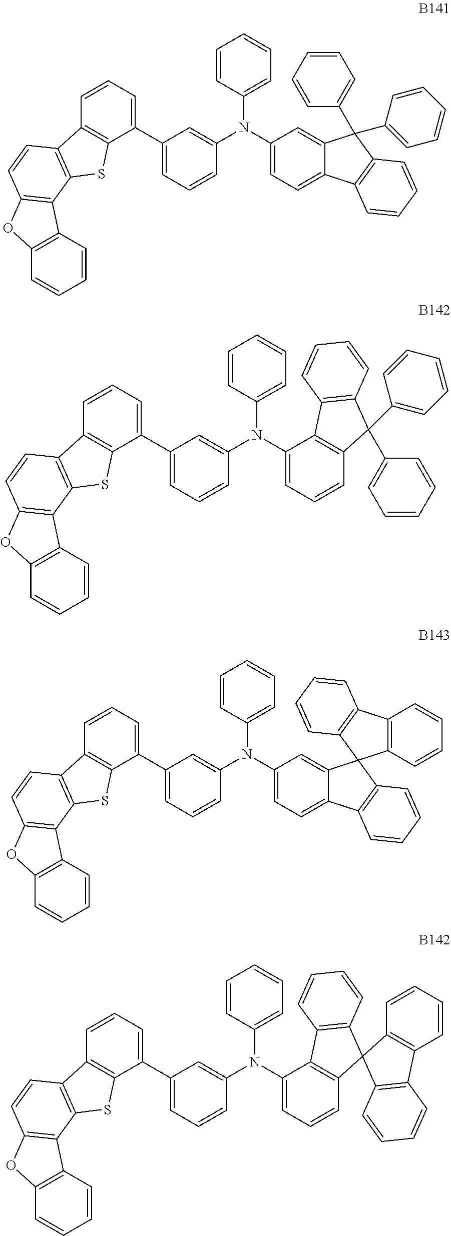 [SMILES: c1ccc(N(c2cccc(-c3cccc4c3sc3c4ccc4oc5ccccc5c43)c2)c2ccc3c(c2)C(c2ccccc2)(c2ccccc2)c2ccccc2-3)cc1 ; c1ccc(N(c2cccc(-c3cccc4c3sc3c4ccc4oc5ccccc5c43)c2)c2cccc3c2-c2ccccc2C3(c2ccccc2)c2ccccc2)cc1 ; c1ccc(N(c2cccc(-c3cccc4c3sc3c4ccc4oc5ccccc5c43)c2)c2ccc3c(c2)C2(c4ccccc4-c4ccccc42)c2ccccc2-3)cc1 ; c1ccc(N(c2cccc(-c3cccc4c3sc3c4ccc4oc5ccccc5c43)c2)c2cccc3c2-c2ccccc2C32c3ccccc3-c3ccccc32)cc1]

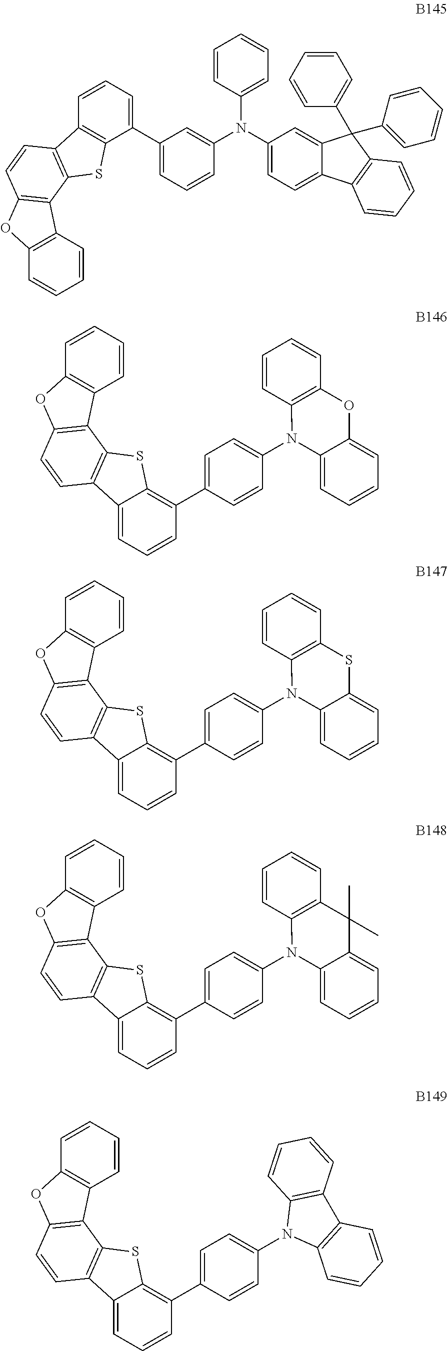 [SMILES: c1ccc(N(c2cccc(-c3cccc4c3sc3c4ccc4oc5ccccc5c43)c2)c2ccc3c(c2)C(c2ccccc2)(c2ccccc2)c2ccccc2-3)cc1 ; c1ccc2c(c1)Oc1ccccc1N2c1ccc(-c2cccc3c2sc2c3ccc3oc4ccccc4c32)cc1 ; c1ccc2c(c1)Sc1ccccc1N2c1ccc(-c2cccc3c2sc2c3ccc3oc4ccccc4c32)cc1 ; CC1(C)c2ccccc2N(c2ccc(-c3cccc4c3sc3c4ccc4oc5ccccc5c43)cc2)c2ccccc21 ; c1ccc2c(c1)oc1ccc3c4cccc(-c5ccc(-n6c7ccccc7c7ccccc76)cc5)c4sc3c12]

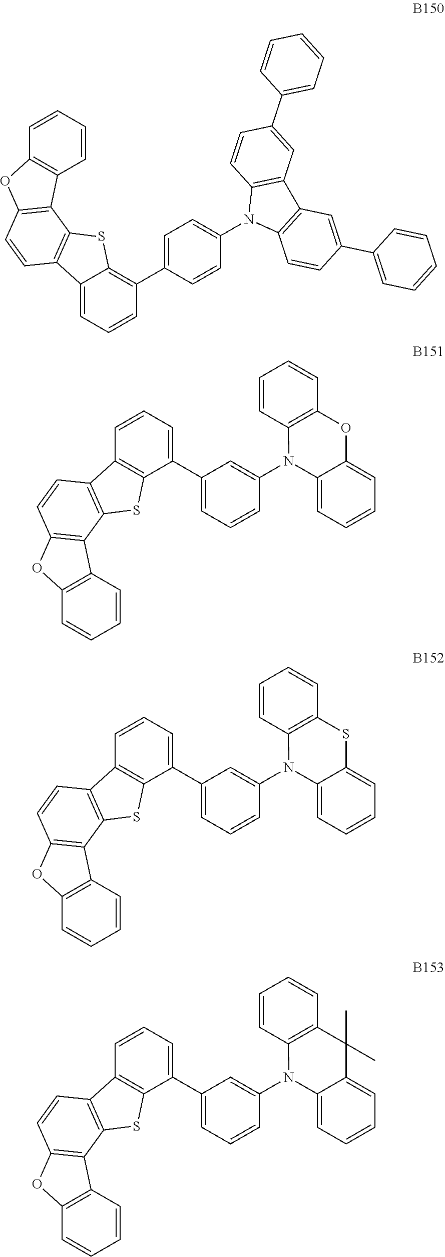 [SMILES: c1ccc(-c2ccc3c(c2)c2cc(-c4ccccc4)ccc2n3-c2ccc(-c3cccc4c3sc3c4ccc4oc5ccccc5c43)cc2)cc1 ; c1cc(-c2cccc3c2sc2c3ccc3oc4ccccc4c32)cc(N2c3ccccc3Oc3ccccc32)c1 ; c1cc(-c2cccc3c2sc2c3ccc3oc4ccccc4c32)cc(N2c3ccccc3Sc3ccccc32)c1 ; CC1(C)c2ccccc2N(c2cccc(-c3cccc4c3sc3c4ccc4oc5ccccc5c43)c2)c2ccccc21]

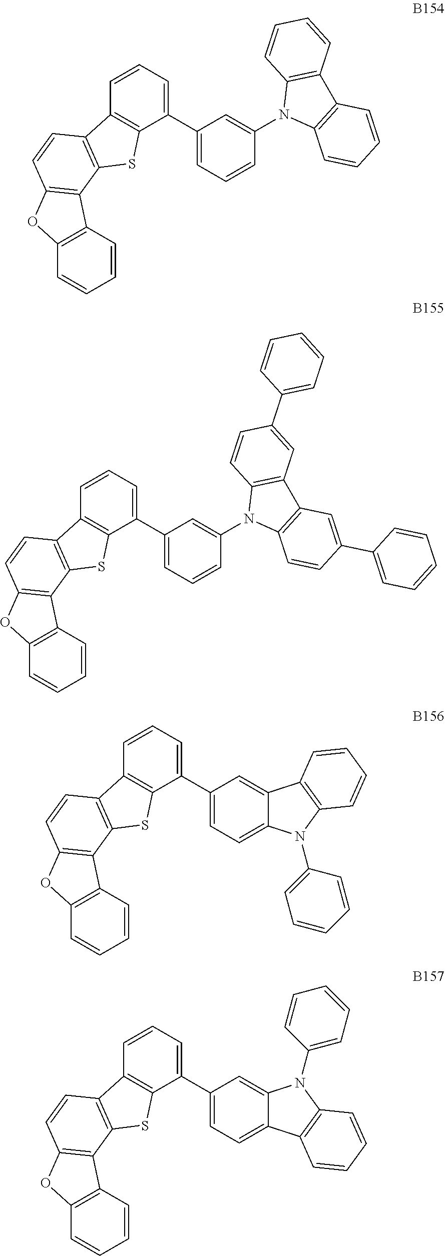 [SMILES: c1cc(-c2cccc3c2sc2c3ccc3oc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3sc3c4ccc4oc5ccccc5c43)c2)cc1 ; c1ccc(-n2c3ccccc3c3cc(-c4cccc5c4sc4c5ccc5oc6ccccc6c54)ccc32)cc1 ; c1ccc(-n2c3ccccc3c3ccc(-c4cccc5c4sc4c5ccc5oc6ccccc6c54)cc32)cc1]

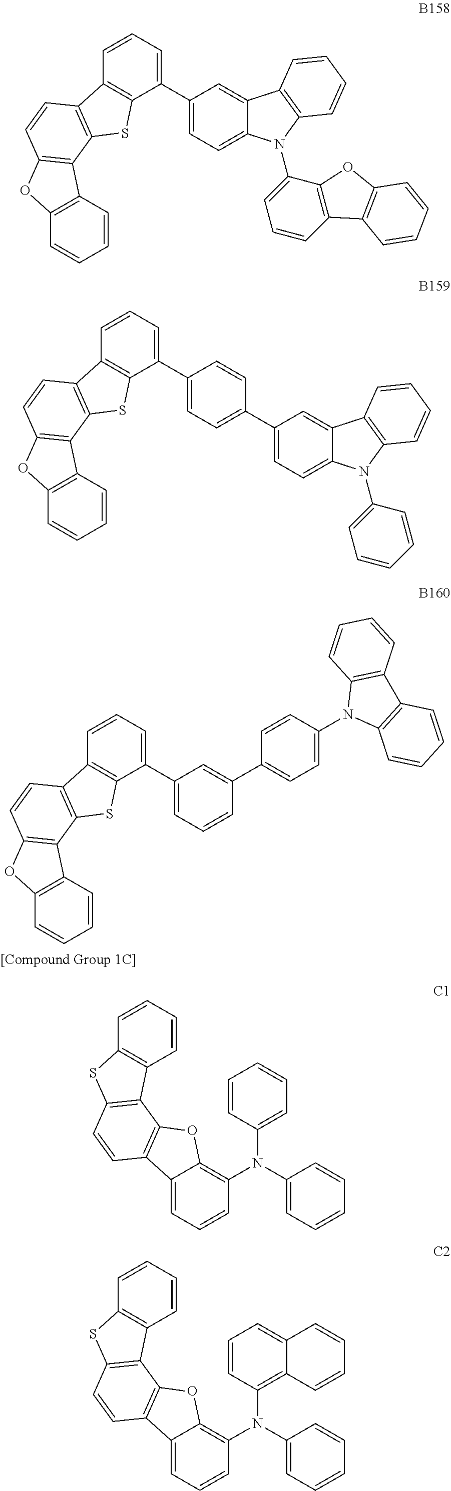 [SMILES: c1ccc2c(c1)oc1c(-n3c4ccccc4c4cc(-c5cccc6c5sc5c6ccc6oc7ccccc7c65)ccc43)cccc12 ; c1ccc(-n2c3ccccc3c3cc(-c4ccc(-c5cccc6c5sc5c6ccc6oc7ccccc7c65)cc4)ccc32)cc1 ; c1cc(-c2ccc(-n3c4ccccc4c4ccccc43)cc2)cc(-c2cccc3c2sc2c3ccc3oc4ccccc4c32)c1 ; c1ccc(N(c2ccccc2)c2cccc3c2oc2c3ccc3sc4ccccc4c32)cc1 ; c1ccc(N(c2cccc3ccccc23)c2cccc3c2oc2c3ccc3sc4ccccc4c32)cc1]

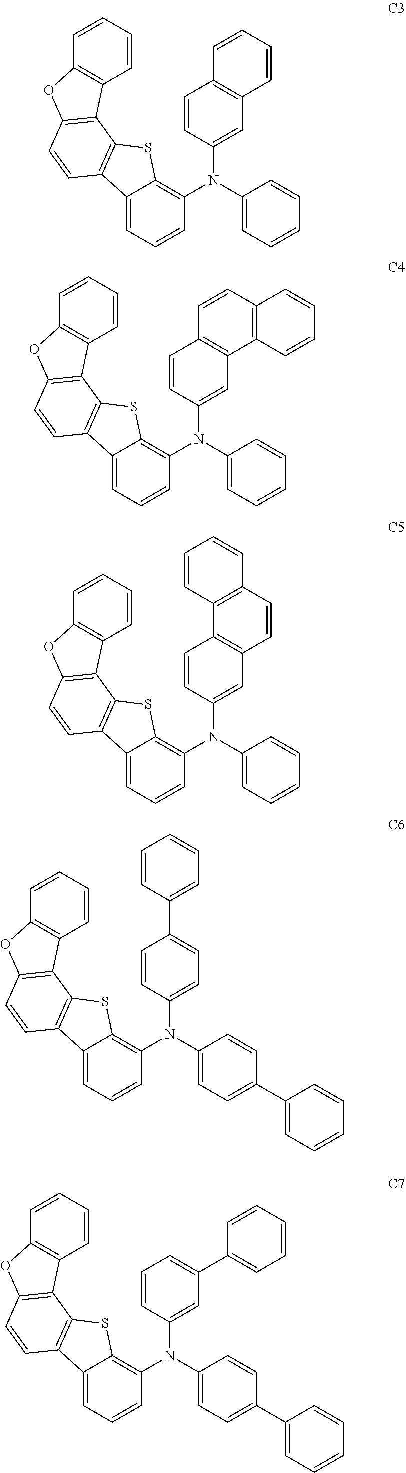

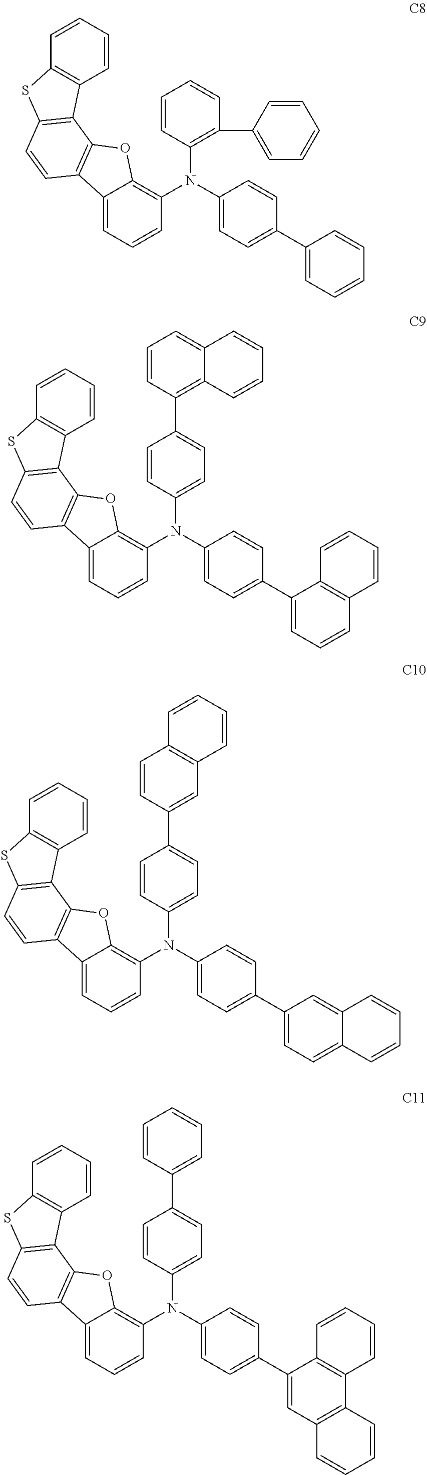 [SMILES: c1ccc(-c2ccc(N(c3ccccc3-c3ccccc3)c3cccc4c3oc3c4ccc4sc5ccccc5c43)cc2)cc1 ; c1ccc2c(-c3ccc(N(c4ccc(-c5cccc6ccccc56)cc4)c4cccc5c4oc4c5ccc5sc6ccccc6c54)cc3)cccc2c1 ; c1ccc2cc(-c3ccc(N(c4ccc(-c5ccc6ccccc6c5)cc4)c4cccc5c4oc4c5ccc5sc6ccccc6c54)cc3)ccc2c1 ; c1ccc(-c2ccc(N(c3ccc(-c4cc5ccccc5c5ccccc45)cc3)c3cccc4c3oc3c4ccc4sc5ccccc5c43)cc2)cc1]

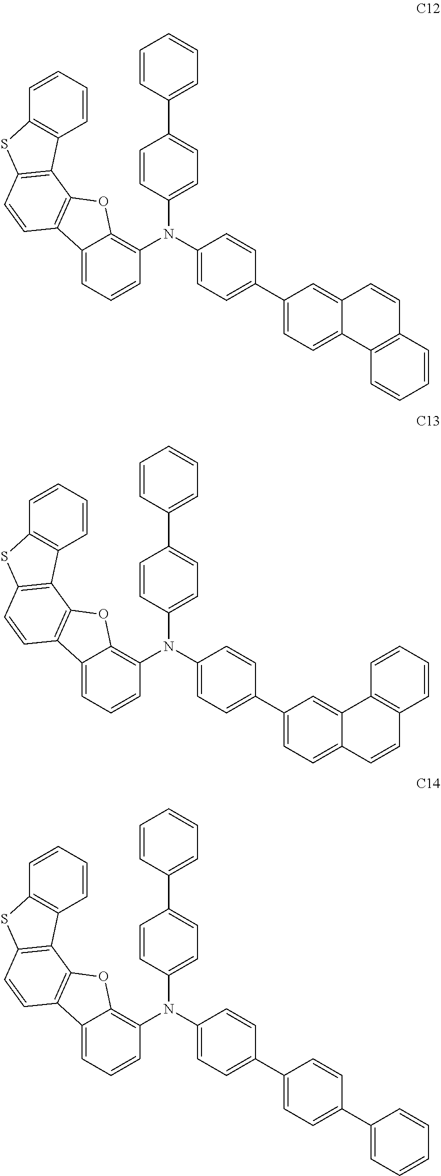 [SMILES: c1ccc(-c2ccc(N(c3ccc(-c4ccc5c(ccc6ccccc65)c4)cc3)c3cccc4c3oc3c4ccc4sc5ccccc5c43)cc2)cc1 ; c1ccc(-c2ccc(N(c3ccc(-c4ccc5ccc6ccccc6c5c4)cc3)c3cccc4c3oc3c4ccc4sc5ccccc5c43)cc2)cc1 ; c1ccc(-c2ccc(-c3ccc(N(c4ccc(-c5ccccc5)cc4)c4cccc5c4oc4c5ccc5sc6ccccc6c54)cc3)cc2)cc1]

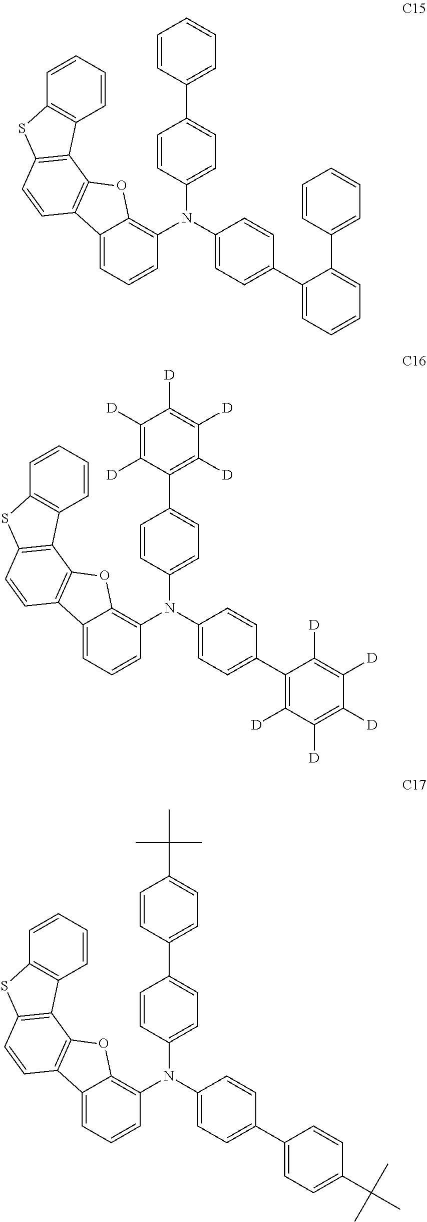 [SMILES: c1ccc(-c2ccc(N(c3ccc(-c4ccccc4-c4ccccc4)cc3)c3cccc4c3oc3c4ccc4sc5ccccc5c43)cc2)cc1 ; [2H]c1c([2H])c([2H])c(-c2ccc(N(c3ccc(-c4c([2H])c([2H])c([2H])c([2H])c4[2H])cc3)c3cccc4c3oc3c4ccc4sc5ccccc5c43)cc2)c([2H])c1[2H] ; CC(C)(C)c1ccc(-c2ccc(N(c3ccc(-c4ccc(C(C)(C)C)cc4)cc3)c3cccc4c3oc3c4ccc4sc5ccccc5c43)cc2)cc1]

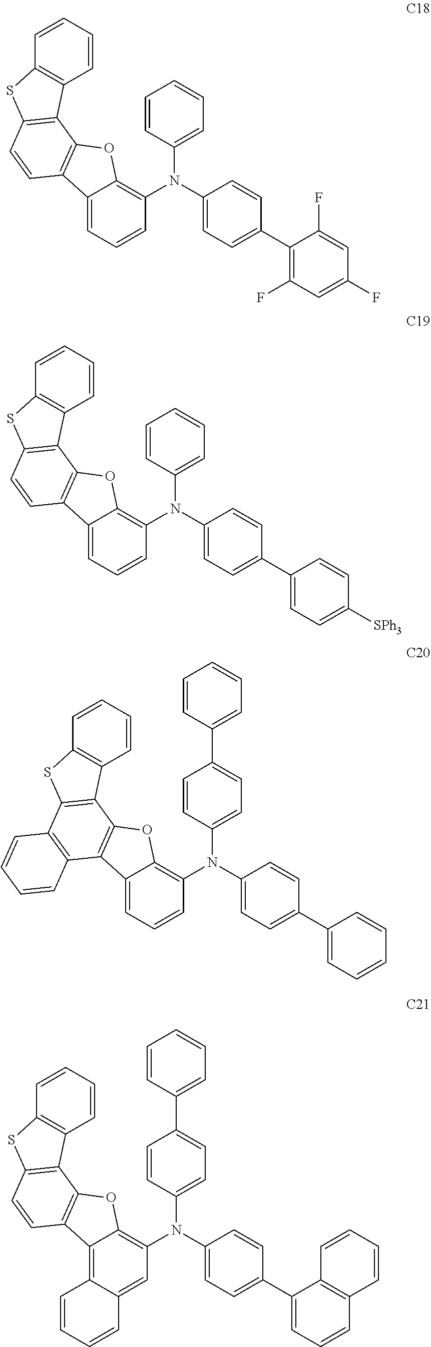

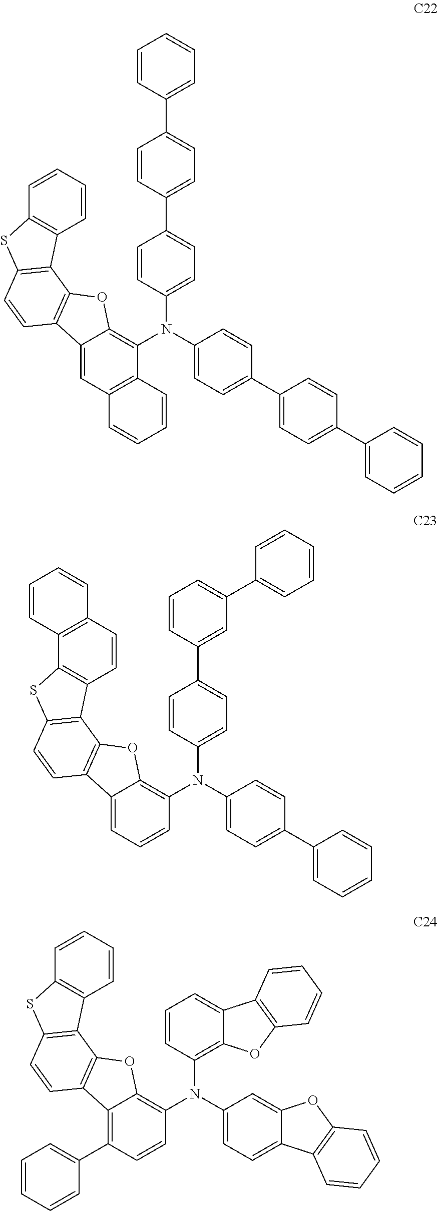 [SMILES: c1ccc(-c2ccc(-c3ccc(N(c4ccc(-c5ccc(-c6ccccc6)cc5)cc4)c4c5ccccc5cc5c4oc4c5ccc5sc6ccccc6c54)cc3)cc2)cc1 ; c1ccc(-c2ccc(N(c3ccc(-c4cccc(-c5ccccc5)c4)cc3)c3cccc4c3oc3c4ccc4sc5c6ccccc6ccc5c43)cc2)cc1 ; c1ccc(-c2ccc(N(c3ccc4c(c3)oc3ccccc34)c3cccc4c3oc3ccccc34)c3oc4c(ccc5sc6ccccc6c54)c23)cc1]

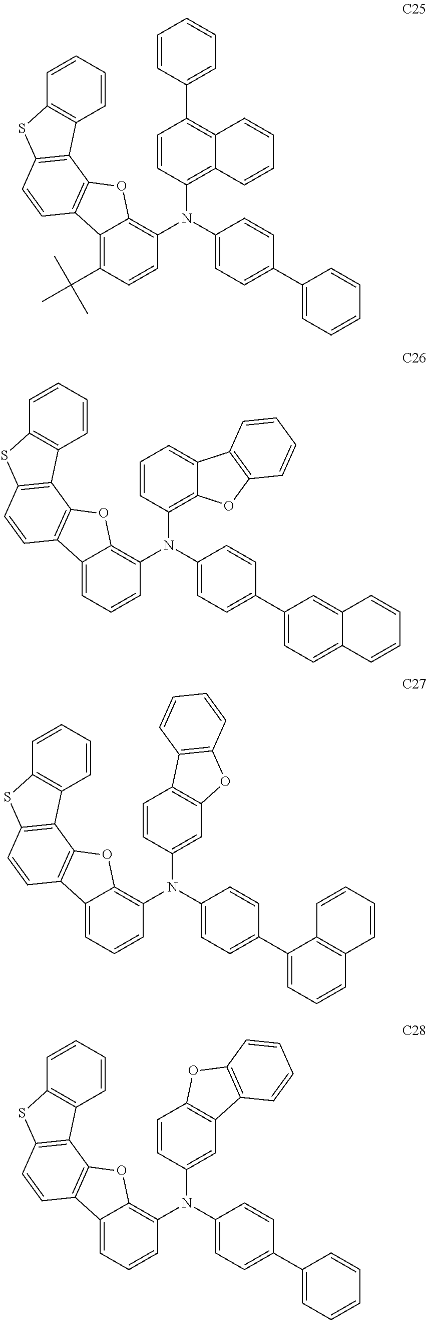 [SMILES: CC(C)(C)c1ccc(N(c2ccc(-c3ccccc3)cc2)c2ccc(-c3ccccc3)c3ccccc23)c2oc3c(ccc4sc5ccccc5c43)c12 ; c1ccc2cc(-c3ccc(N(c4cccc5c4oc4ccccc45)c4cccc5c4oc4c5ccc5sc6ccccc6c54)cc3)ccc2c1 ; c1ccc2c(-c3ccc(N(c4ccc5c(c4)oc4ccccc45)c4cccc5c4oc4c5ccc5sc6ccccc6c54)cc3)cccc2c1 ; c1ccc(-c2ccc(N(c3ccc4oc5ccccc5c4c3)c3cccc4c3oc3c4ccc4sc5ccccc5c43)cc2)cc1]

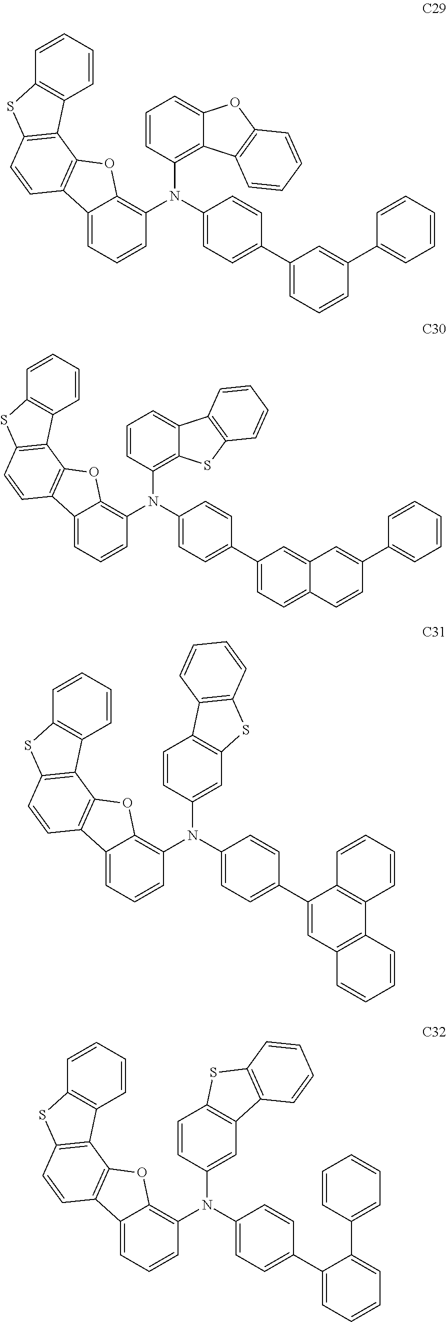 [SMILES: c1ccc(-c2cccc(-c3ccc(N(c4cccc5c4oc4c5ccc5sc6ccccc6c54)c4cccc5oc6ccccc6c45)cc3)c2)cc1 ; c1ccc(-c2ccc3ccc(-c4ccc(N(c5cccc6c5oc5c6ccc6sc7ccccc7c65)c5cccc6c5sc5ccccc56)cc4)cc3c2)cc1 ; c1ccc2c(c1)cc(-c1ccc(N(c3ccc4c(c3)sc3ccccc34)c3cccc4c3oc3c4ccc4sc5ccccc5c43)cc1)c1ccccc12 ; c1ccc(-c2ccccc2-c2ccc(N(c3ccc4sc5ccccc5c4c3)c3cccc4c3oc3c4ccc4sc5ccccc5c43)cc2)cc1]

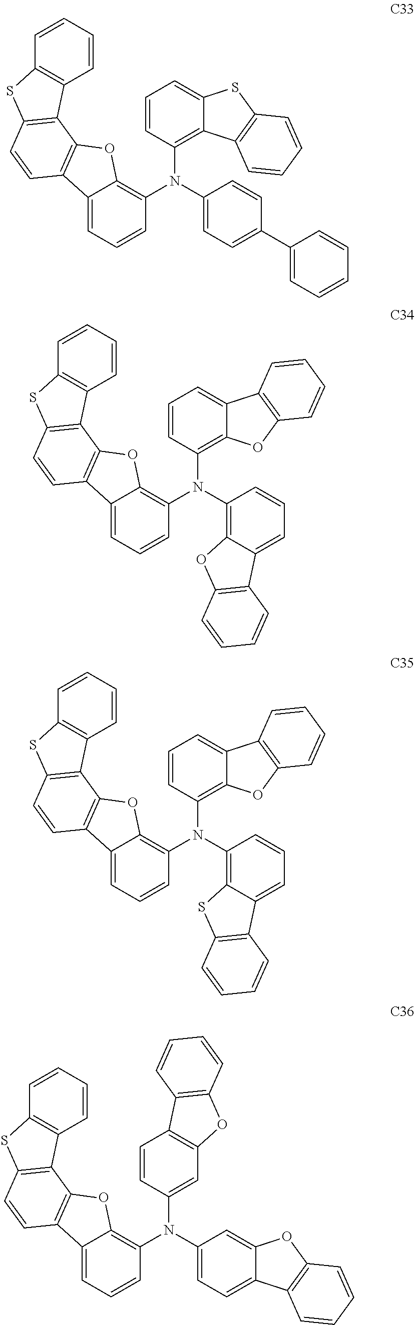

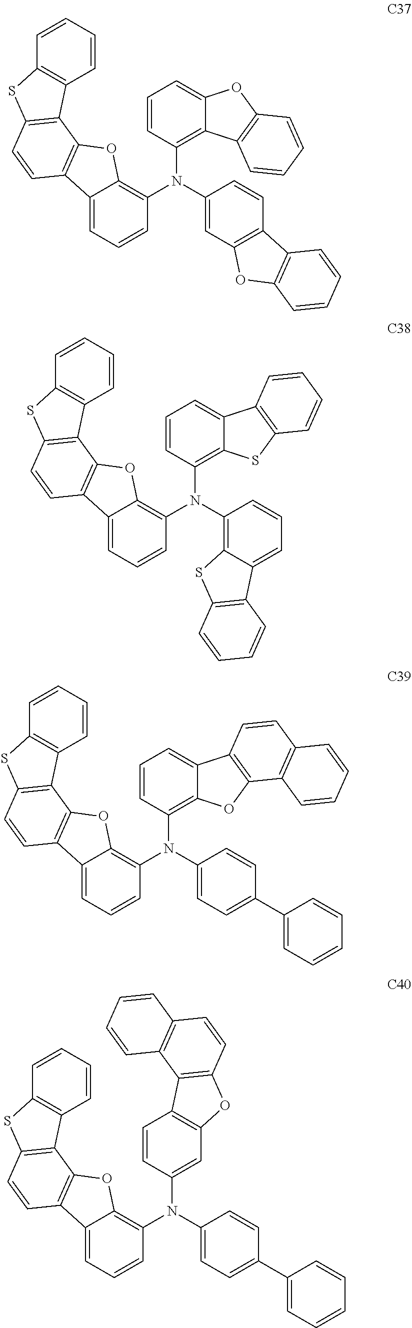 [SMILES: c1ccc2c(c1)oc1cc(N(c3cccc4c3oc3c4ccc4sc5ccccc5c43)c3cccc4oc5ccccc5c34)ccc12 ; c1ccc2c(c1)sc1c(N(c3cccc4c3oc3c4ccc4sc5ccccc5c43)c3cccc4c3sc3ccccc34)cccc12 ; c1ccc(-c2ccc(N(c3cccc4c3oc3c5ccccc5ccc43)c3cccc4c3oc3c4ccc4sc5ccccc5c43)cc2)cc1 ; c1ccc(-c2ccc(N(c3ccc4c(c3)oc3ccc5ccccc5c34)c3cccc4c3oc3c4ccc4sc5ccccc5c43)cc2)cc1]

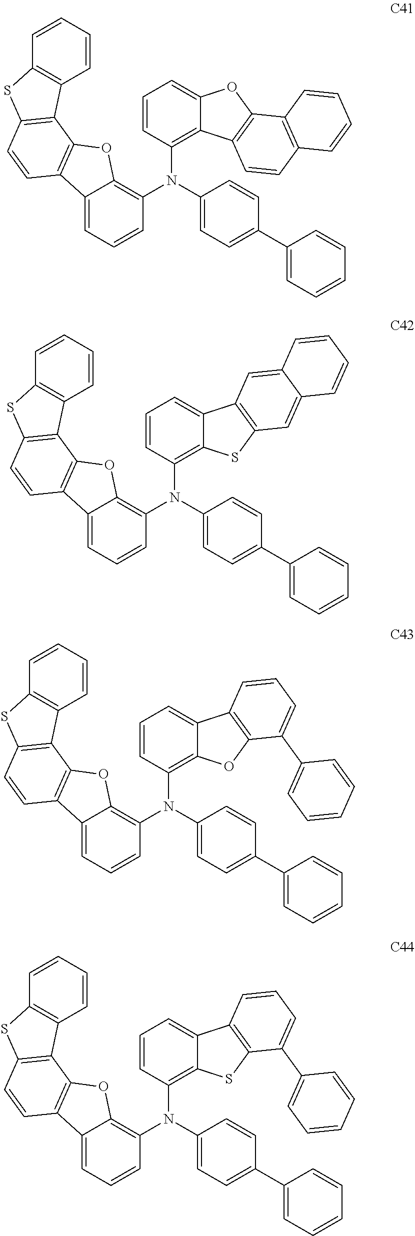

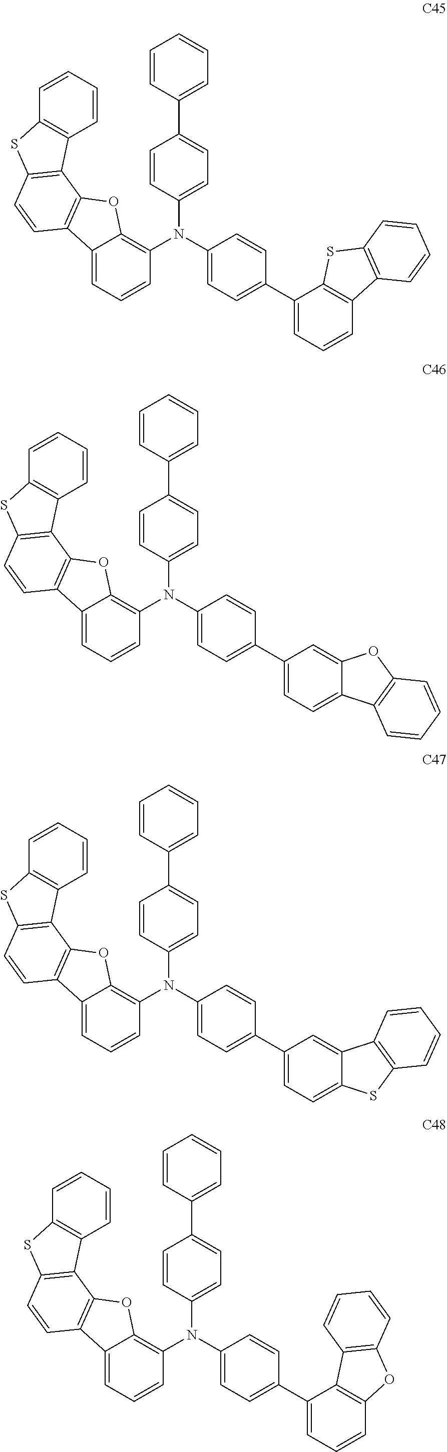 [SMILES: c1ccc(-c2ccc(N(c3ccc(-c4cccc5c4sc4ccccc45)cc3)c3cccc4c3oc3c4ccc4sc5ccccc5c43)cc2)cc1 ; c1ccc(-c2ccc(N(c3ccc(-c4ccc5c(c4)oc4ccccc45)cc3)c3cccc4c3oc3c4ccc4sc5ccccc5c43)cc2)cc1 ; c1ccc(-c2ccc(N(c3ccc(-c4ccc5sc6ccccc6c5c4)cc3)c3cccc4c3oc3c4ccc4sc5ccccc5c43)cc2)cc1 ; c1ccc(-c2ccc(N(c3ccc(-c4cccc5oc6ccccc6c45)cc3)c3cccc4c3oc3c4ccc4sc5ccccc5c43)cc2)cc1]

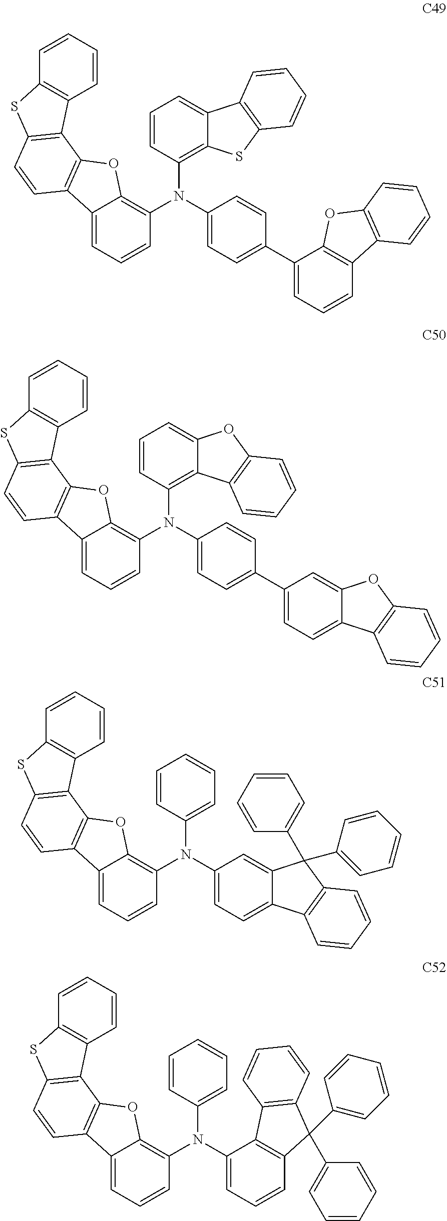 [SMILES: c1ccc2c(c1)oc1c(-c3ccc(N(c4cccc5c4oc4c5ccc5sc6ccccc6c54)c4cccc5c4sc4ccccc45)cc3)cccc12 ; c1ccc2c(c1)oc1cc(-c3ccc(N(c4cccc5c4oc4c5ccc5sc6ccccc6c54)c4cccc5oc6ccccc6c45)cc3)ccc12 ; c1ccc(N(c2ccc3c(c2)C(c2ccccc2)(c2ccccc2)c2ccccc2-3)c2cccc3c2oc2c3ccc3sc4ccccc4c32)cc1 ; c1ccc(N(c2cccc3c2-c2ccccc2C3(c2ccccc2)c2ccccc2)c2cccc3c2oc2c3ccc3sc4ccccc4c32)cc1]

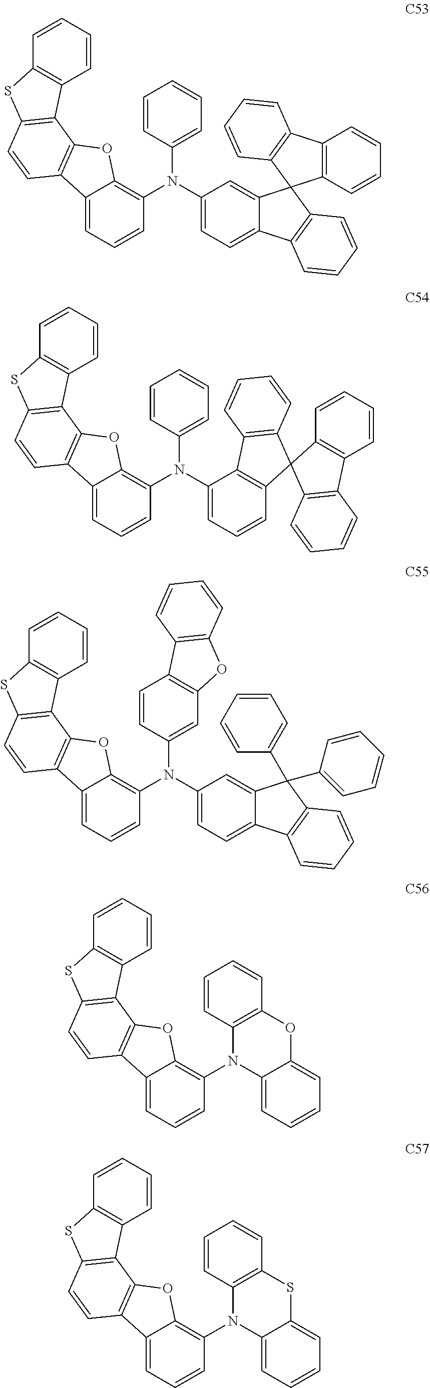 [SMILES: c1ccc(N(c2ccc3c(c2)C2(c4ccccc4-c4ccccc42)c2ccccc2-3)c2cccc3c2oc2c3ccc3sc4ccccc4c32)cc1 ; c1ccc(N(c2cccc3c2-c2ccccc2C32c3ccccc3-c3ccccc32)c2cccc3c2oc2c3ccc3sc4ccccc4c32)cc1 ; c1ccc(C2(c3ccccc3)c3ccccc3-c3ccc(N(c4ccc5c(c4)oc4ccccc45)c4cccc5c4oc4c5ccc5sc6ccccc6c54)cc32)cc1 ; c1ccc2c(c1)Oc1ccccc1N2c1cccc2c1oc1c2ccc2sc3ccccc3c21 ; c1ccc2c(c1)Sc1ccccc1N2c1cccc2c1oc1c2ccc2sc3ccccc3c21]

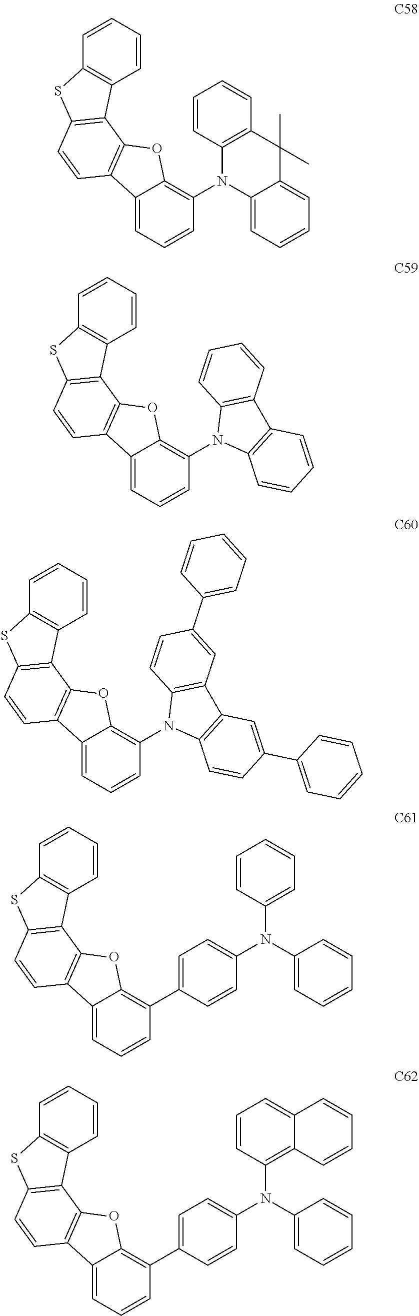

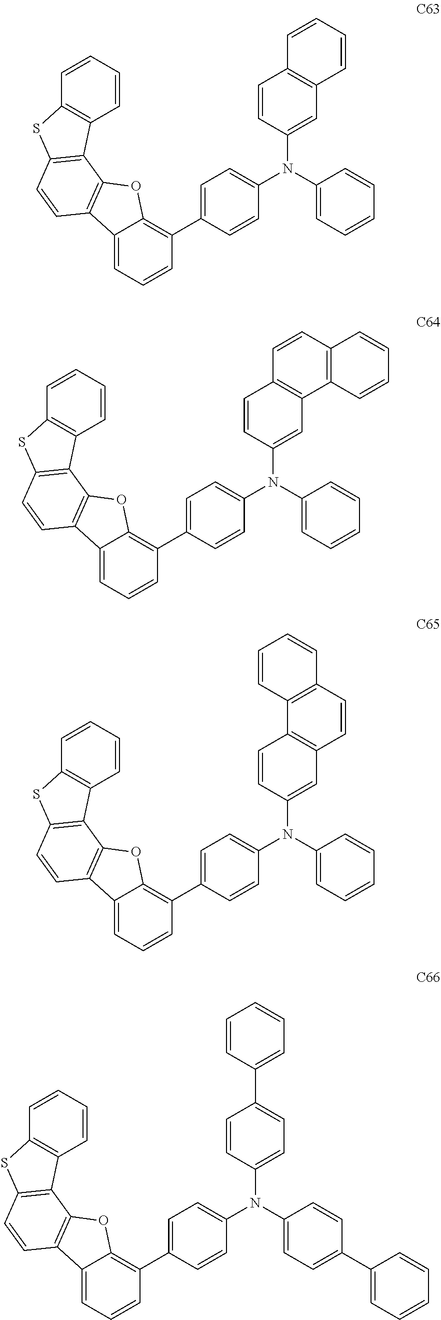 [SMILES: c1ccc(N(c2ccc(-c3cccc4c3oc3c4ccc4sc5ccccc5c43)cc2)c2ccc3ccccc3c2)cc1 ; c1ccc(N(c2ccc(-c3cccc4c3oc3c4ccc4sc5ccccc5c43)cc2)c2ccc3ccc4ccccc4c3c2)cc1 ; c1ccc(N(c2ccc(-c3cccc4c3oc3c4ccc4sc5ccccc5c43)cc2)c2ccc3c(ccc4ccccc43)c2)cc1 ; c1ccc(-c2ccc(N(c3ccc(-c4ccccc4)cc3)c3ccc(-c4cccc5c4oc4c5ccc5sc6ccccc6c54)cc3)cc2)cc1]

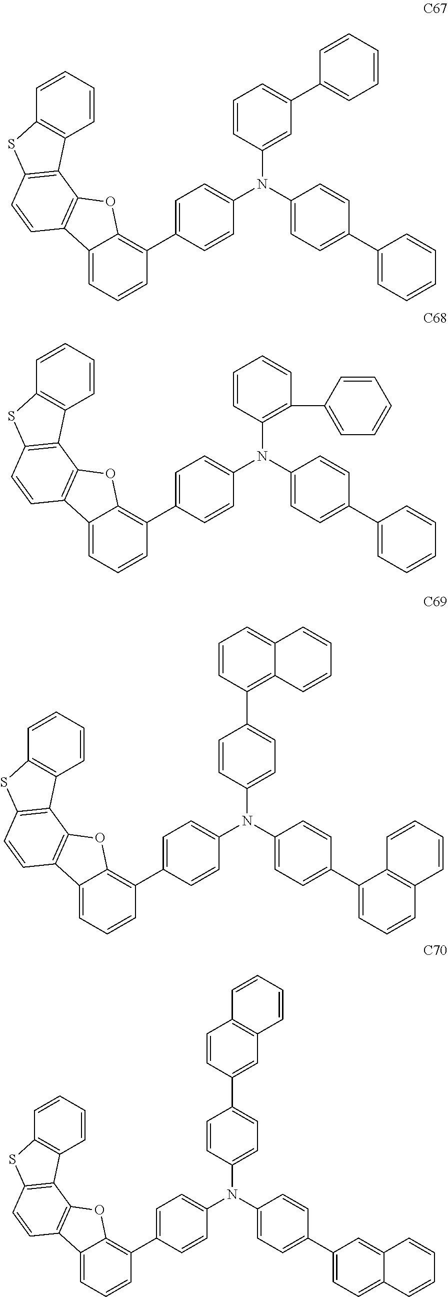 [SMILES: c1ccc(-c2ccc(N(c3ccc(-c4cccc5c4oc4c5ccc5sc6ccccc6c54)cc3)c3cccc(-c4ccccc4)c3)cc2)cc1 ; c1ccc(-c2ccc(N(c3ccc(-c4cccc5c4oc4c5ccc5sc6ccccc6c54)cc3)c3ccccc3-c3ccccc3)cc2)cc1 ; c1ccc2c(-c3ccc(N(c4ccc(-c5cccc6ccccc56)cc4)c4ccc(-c5cccc6c5oc5c6ccc6sc7ccccc7c65)cc4)cc3)cccc2c1 ; c1ccc2cc(-c3ccc(N(c4ccc(-c5ccc6ccccc6c5)cc4)c4ccc(-c5cccc6c5oc5c6ccc6sc7ccccc7c65)cc4)cc3)ccc2c1]

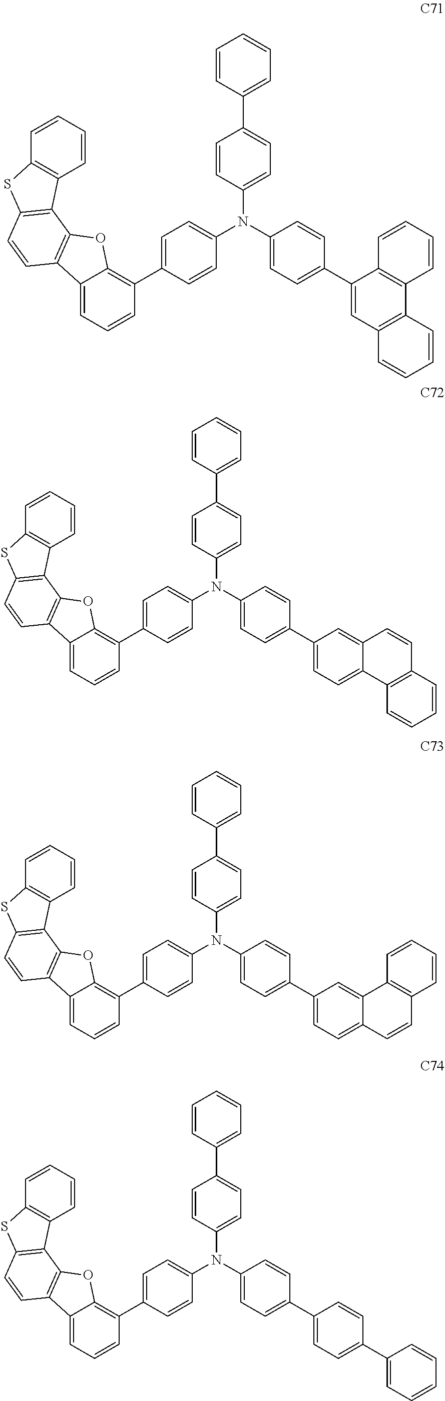 [SMILES: c1ccc(-c2ccc(N(c3ccc(-c4cc5ccccc5c5ccccc45)cc3)c3ccc(-c4cccc5c4oc4c5ccc5sc6ccccc6c54)cc3)cc2)cc1 ; c1ccc(-c2ccc(N(c3ccc(-c4ccc5c(ccc6ccccc65)c4)cc3)c3ccc(-c4cccc5c4oc4c5ccc5sc6ccccc6c54)cc3)cc2)cc1 ; c1ccc(-c2ccc(N(c3ccc(-c4ccc5ccc6ccccc6c5c4)cc3)c3ccc(-c4cccc5c4oc4c5ccc5sc6ccccc6c54)cc3)cc2)cc1 ; c1ccc(-c2ccc(-c3ccc(N(c4ccc(-c5ccccc5)cc4)c4ccc(-c5cccc6c5oc5c6ccc6sc7ccccc7c65)cc4)cc3)cc2)cc1]

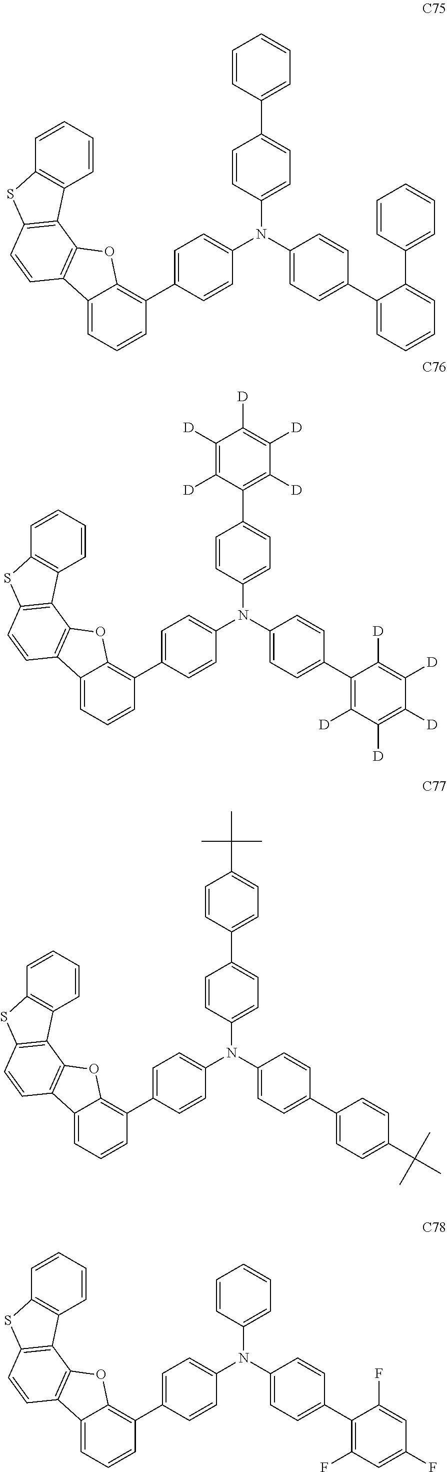 [SMILES: c1ccc(-c2ccc(N(c3ccc(-c4ccccc4-c4ccccc4)cc3)c3ccc(-c4cccc5c4oc4c5ccc5sc6ccccc6c54)cc3)cc2)cc1 ; [2H]c1c([2H])c([2H])c(-c2ccc(N(c3ccc(-c4c([2H])c([2H])c([2H])c([2H])c4[2H])cc3)c3ccc(-c4cccc5c4oc4c5ccc5sc6ccccc6c54)cc3)cc2)c([2H])c1[2H] ; CC(C)(C)c1ccc(-c2ccc(N(c3ccc(-c4ccc(C(C)(C)C)cc4)cc3)c3ccc(-c4cccc5c4oc4c5ccc5sc6ccccc6c54)cc3)cc2)cc1 ; Fc1cc(F)c(-c2ccc(N(c3ccccc3)c3ccc(-c4cccc5c4oc4c5ccc5sc6ccccc6c54)cc3)cc2)c(F)c1]

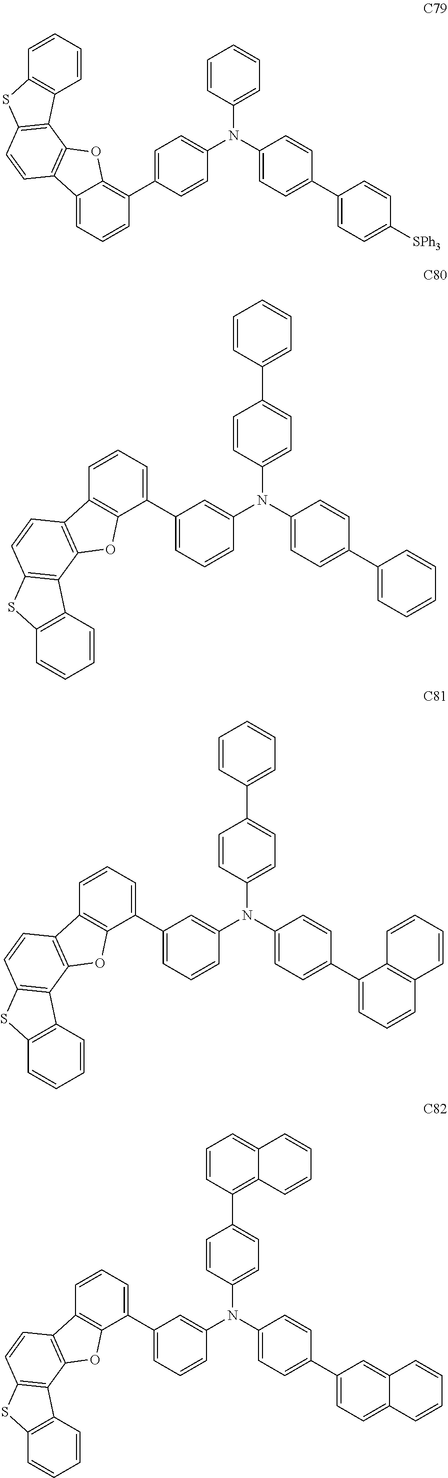

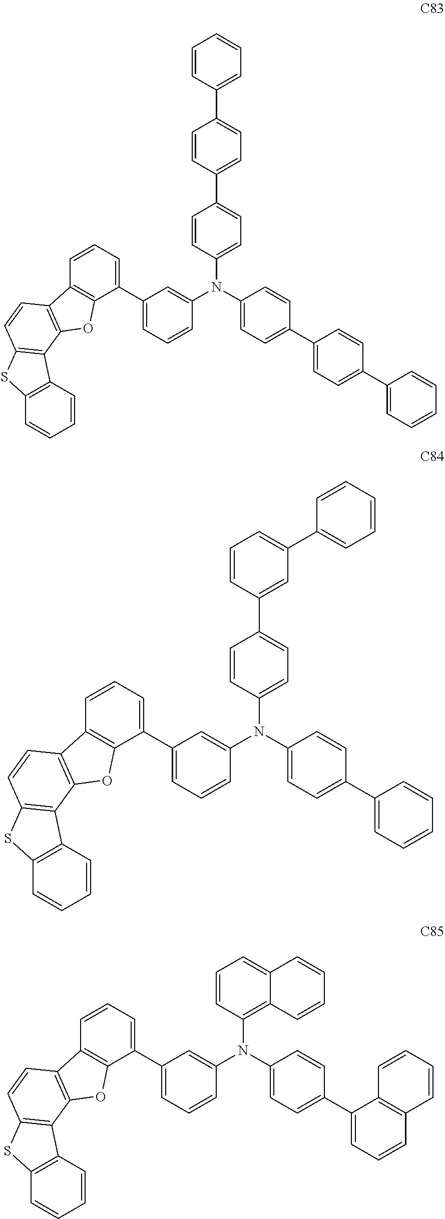 [SMILES: c1ccc(-c2ccc(-c3ccc(N(c4ccc(-c5ccc(-c6ccccc6)cc5)cc4)c4cccc(-c5cccc6c5oc5c6ccc6sc7ccccc7c65)c4)cc3)cc2)cc1 ; c1ccc(-c2ccc(N(c3ccc(-c4cccc(-c5ccccc5)c4)cc3)c3cccc(-c4cccc5c4oc4c5ccc5sc6ccccc6c54)c3)cc2)cc1 ; c1cc(-c2cccc3c2oc2c3ccc3sc4ccccc4c32)cc(N(c2ccc(-c3cccc4ccccc34)cc2)c2cccc3ccccc23)c1]

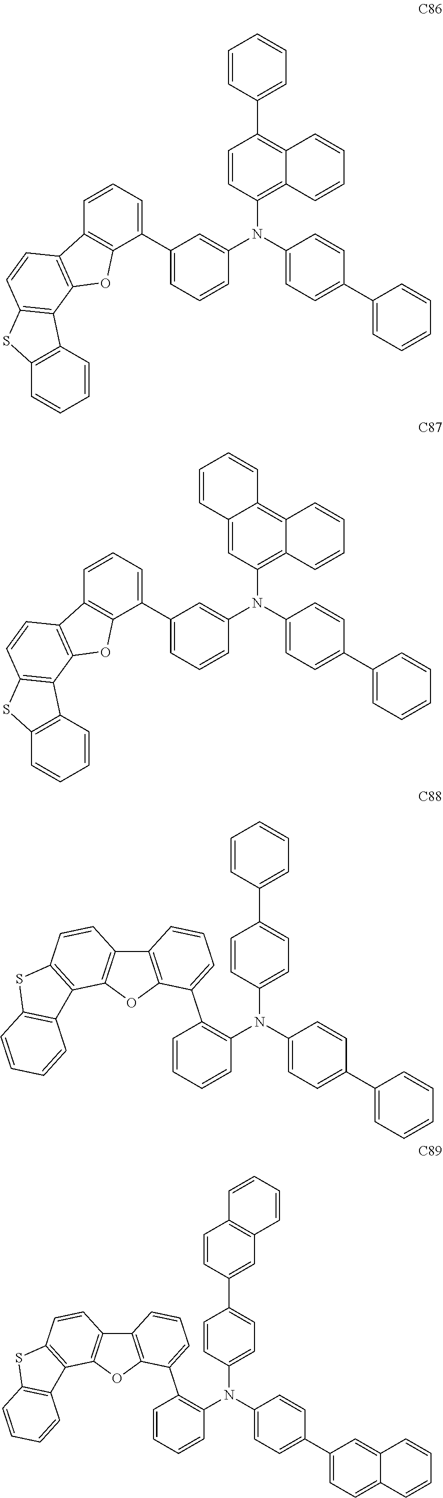 [SMILES: c1ccc(-c2ccc(N(c3cccc(-c4cccc5c4oc4c5ccc5sc6ccccc6c54)c3)c3ccc(-c4ccccc4)c4ccccc34)cc2)cc1 ; c1ccc(-c2ccc(N(c3cccc(-c4cccc5c4oc4c5ccc5sc6ccccc6c54)c3)c3cc4ccccc4c4ccccc34)cc2)cc1 ; c1ccc(-c2ccc(N(c3ccc(-c4ccccc4)cc3)c3ccccc3-c3cccc4c3oc3c4ccc4sc5ccccc5c43)cc2)cc1 ; c1ccc(N(c2ccc(-c3ccc4ccccc4c3)cc2)c2ccc(-c3ccc4ccccc4c3)cc2)c(-c2cccc3c2oc2c3ccc3sc4ccccc4c32)c1]

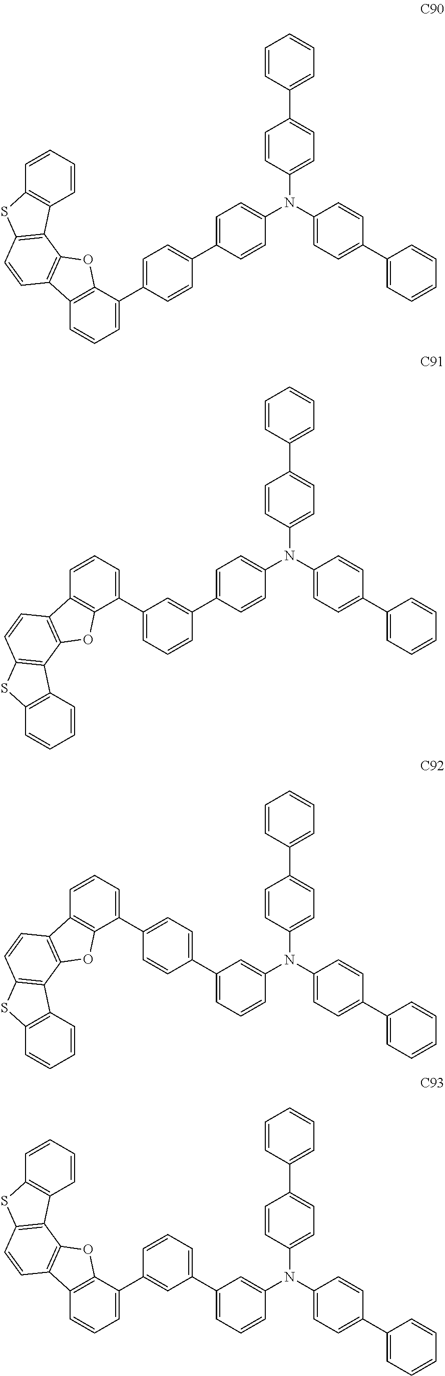 [SMILES: c1ccc(-c2ccc(N(c3ccc(-c4ccccc4)cc3)c3ccc(-c4ccc(-c5cccc6c5oc5c6ccc6sc7ccccc7c65)cc4)cc3)cc2)cc1 ; c1ccc(-c2ccc(N(c3ccc(-c4ccccc4)cc3)c3ccc(-c4cccc(-c5cccc6c5oc5c6ccc6sc7ccccc7c65)c4)cc3)cc2)cc1 ; c1ccc(-c2ccc(N(c3ccc(-c4ccccc4)cc3)c3cccc(-c4ccc(-c5cccc6c5oc5c6ccc6sc7ccccc7c65)cc4)c3)cc2)cc1 ; c1ccc(-c2ccc(N(c3ccc(-c4ccccc4)cc3)c3cccc(-c4cccc(-c5cccc6c5oc5c6ccc6sc7ccccc7c65)c4)c3)cc2)cc1]

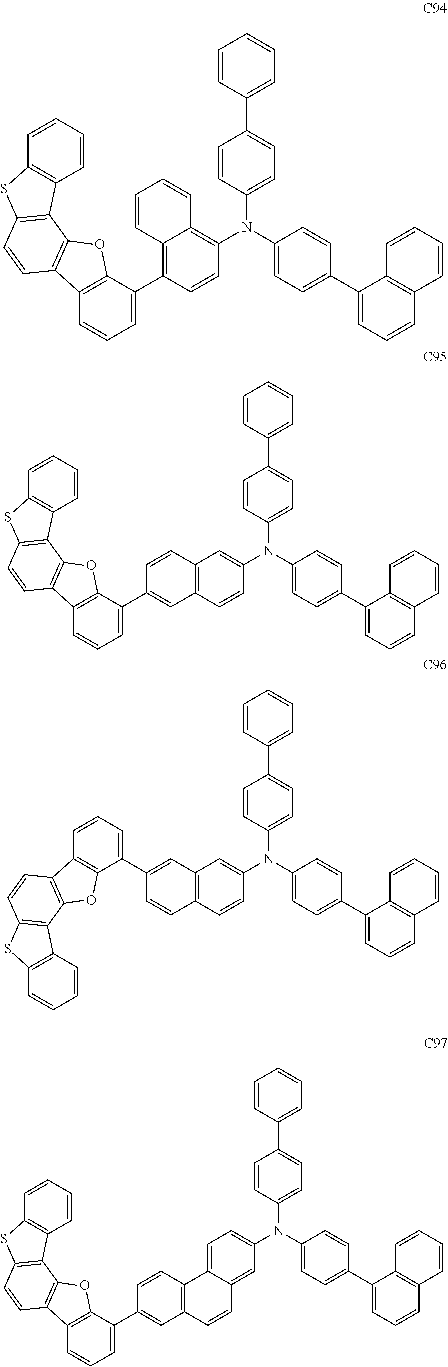 [SMILES: c1ccc(-c2ccc(N(c3ccc(-c4cccc5ccccc45)cc3)c3ccc(-c4cccc5c4oc4c5ccc5sc6ccccc6c54)c4ccccc34)cc2)cc1 ; c1ccc(-c2ccc(N(c3ccc(-c4cccc5ccccc45)cc3)c3ccc4cc(-c5cccc6c5oc5c6ccc6sc7ccccc7c65)ccc4c3)cc2)cc1 ; c1ccc(-c2ccc(N(c3ccc(-c4cccc5ccccc45)cc3)c3ccc4ccc(-c5cccc6c5oc5c6ccc6sc7ccccc7c65)cc4c3)cc2)cc1 ; c1ccc(-c2ccc(N(c3ccc(-c4cccc5ccccc45)cc3)c3ccc4c(ccc5cc(-c6cccc7c6oc6c7ccc7sc8ccccc8c76)ccc54)c3)cc2)cc1]

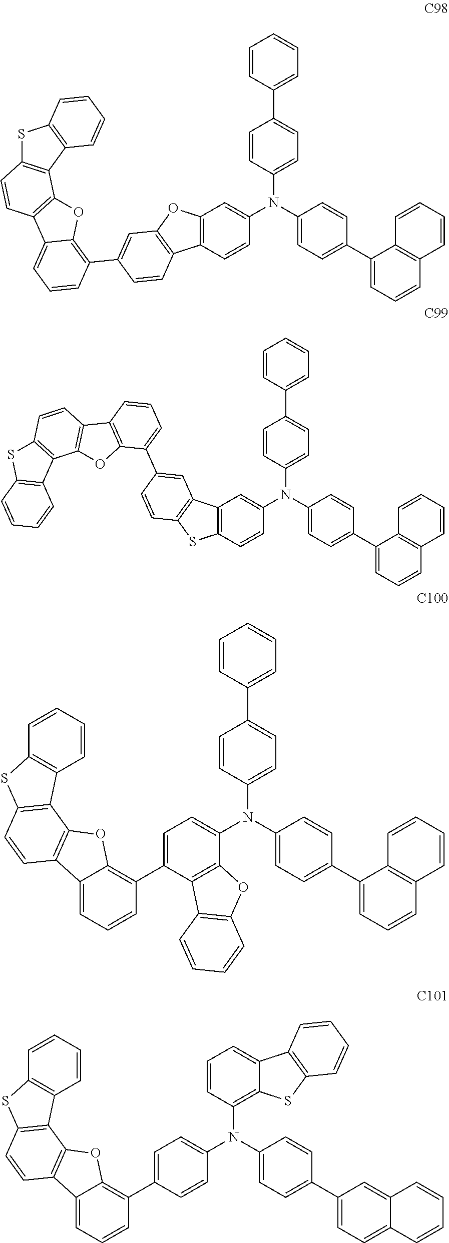 [SMILES: c1ccc(-c2ccc(N(c3ccc(-c4cccc5ccccc45)cc3)c3ccc4c(c3)oc3cc(-c5cccc6c5oc5c6ccc6sc7ccccc7c65)ccc34)cc2)cc1 ; c1ccc(-c2ccc(N(c3ccc(-c4cccc5ccccc45)cc3)c3ccc4sc5ccc(-c6cccc7c6oc6c7ccc7sc8ccccc8c76)cc5c4c3)cc2)cc1 ; c1ccc(-c2ccc(N(c3ccc(-c4cccc5ccccc45)cc3)c3ccc(-c4cccc5c4oc4c5ccc5sc6ccccc6c54)c4c3oc3ccccc34)cc2)cc1 ; c1ccc2cc(-c3ccc(N(c4ccc(-c5cccc6c5oc5c6ccc6sc7ccccc7c65)cc4)c4cccc5c4sc4ccccc45)cc3)ccc2c1]

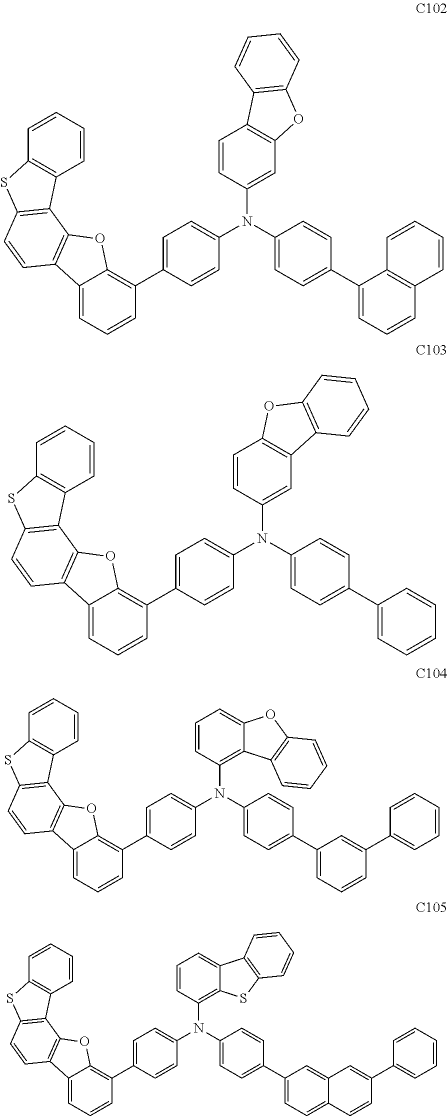 [SMILES: c1ccc2c(-c3ccc(N(c4ccc(-c5cccc6c5oc5c6ccc6sc7ccccc7c65)cc4)c4ccc5c(c4)oc4ccccc45)cc3)cccc2c1 ; c1ccc(-c2ccc(N(c3ccc(-c4cccc5c4oc4c5ccc5sc6ccccc6c54)cc3)c3ccc4oc5ccccc5c4c3)cc2)cc1 ; c1ccc(-c2cccc(-c3ccc(N(c4ccc(-c5cccc6c5oc5c6ccc6sc7ccccc7c65)cc4)c4cccc5oc6ccccc6c45)cc3)c2)cc1 ; c1ccc(-c2ccc3ccc(-c4ccc(N(c5ccc(-c6cccc7c6oc6c7ccc7sc8ccccc8c76)cc5)c5cccc6c5sc5ccccc56)cc4)cc3c2)cc1]

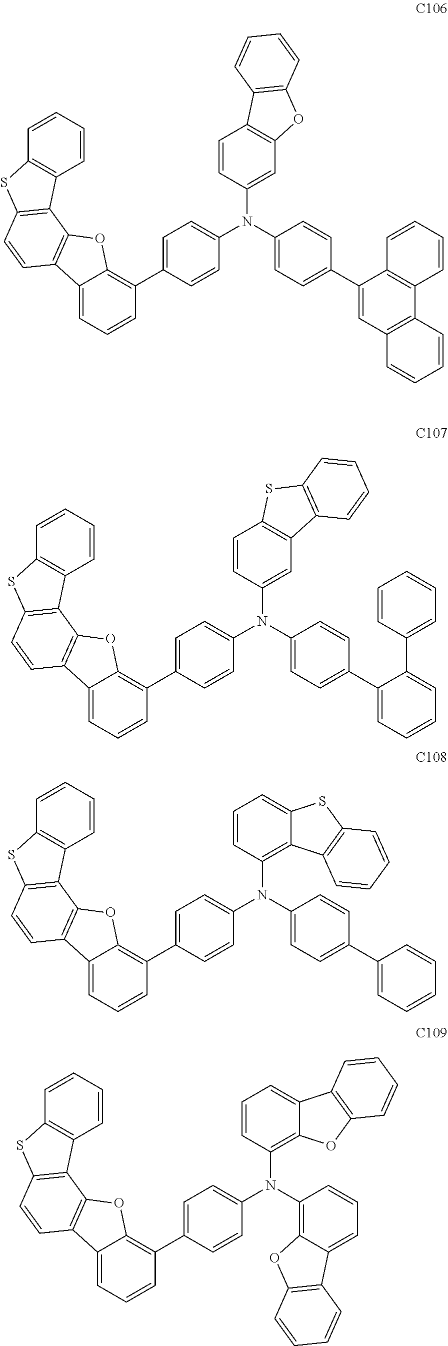 [SMILES: c1ccc2c(c1)cc(-c1ccc(N(c3ccc(-c4cccc5c4oc4c5ccc5sc6ccccc6c54)cc3)c3ccc4c(c3)oc3ccccc34)cc1)c1ccccc12 ; c1ccc(-c2ccccc2-c2ccc(N(c3ccc(-c4cccc5c4oc4c5ccc5sc6ccccc6c54)cc3)c3ccc4sc5ccccc5c4c3)cc2)cc1 ; c1ccc(-c2ccc(N(c3ccc(-c4cccc5c4oc4c5ccc5sc6ccccc6c54)cc3)c3cccc4sc5ccccc5c34)cc2)cc1 ; c1ccc2c(c1)oc1c(N(c3ccc(-c4cccc5c4oc4c5ccc5sc6ccccc6c54)cc3)c3cccc4c3oc3ccccc34)cccc12]

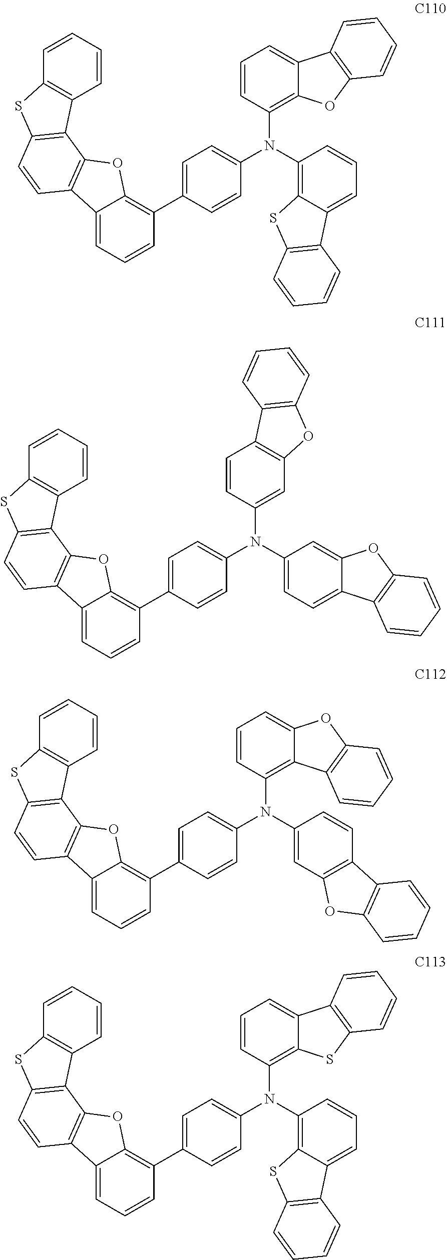

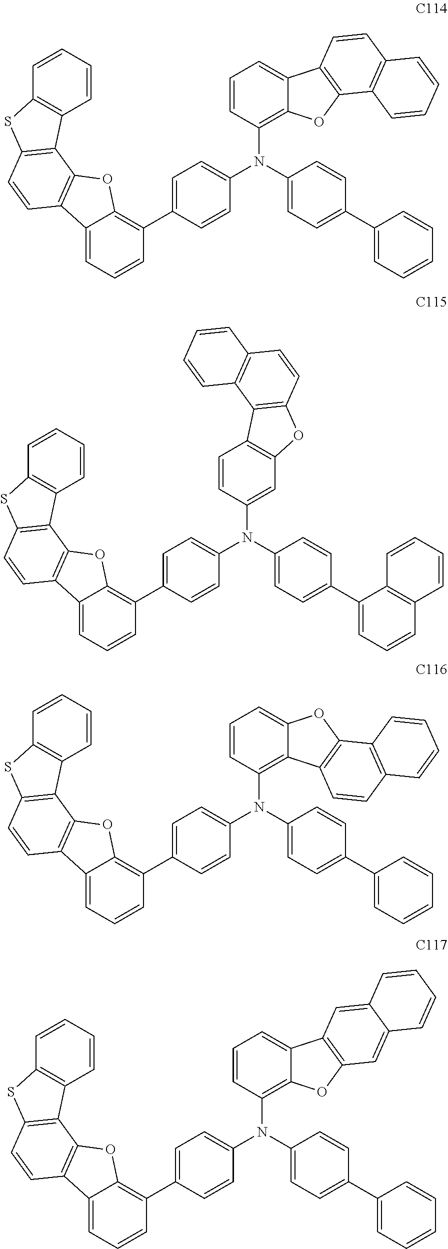 [SMILES: c1ccc(-c2ccc(N(c3ccc(-c4cccc5c4oc4c5ccc5sc6ccccc6c54)cc3)c3cccc4c3oc3c5ccccc5ccc43)cc2)cc1 ; c1ccc2c(-c3ccc(N(c4ccc(-c5cccc6c5oc5c6ccc6sc7ccccc7c65)cc4)c4ccc5c(c4)oc4ccc6ccccc6c45)cc3)cccc2c1 ; c1ccc(-c2ccc(N(c3ccc(-c4cccc5c4oc4c5ccc5sc6ccccc6c54)cc3)c3cccc4oc5c6ccccc6ccc5c34)cc2)cc1 ; c1ccc(-c2ccc(N(c3ccc(-c4cccc5c4oc4c5ccc5sc6ccccc6c54)cc3)c3cccc4c3oc3cc5ccccc5cc34)cc2)cc1]

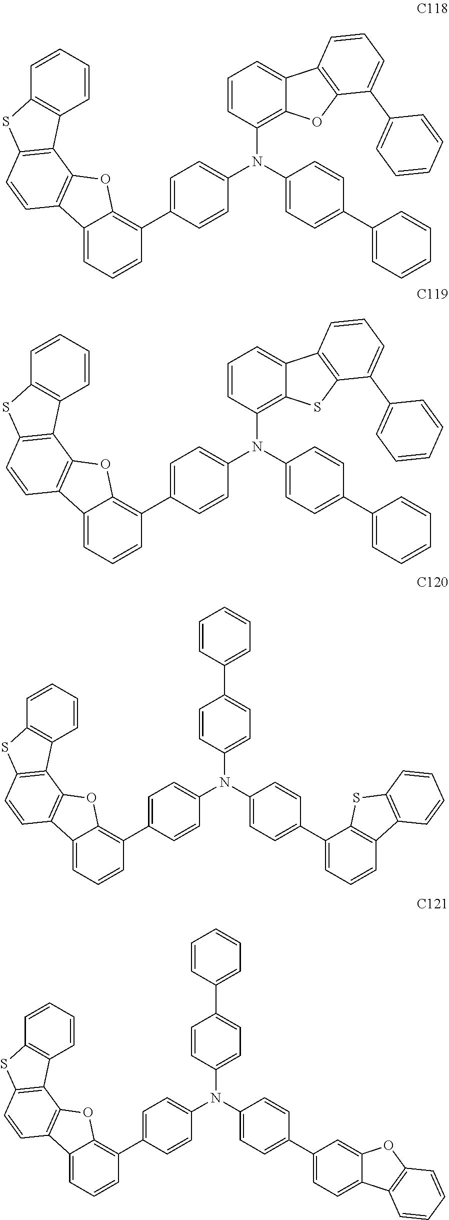 [SMILES: c1ccc(-c2ccc(N(c3ccc(-c4cccc5c4oc4c5ccc5sc6ccccc6c54)cc3)c3cccc4c3oc3c(-c5ccccc5)cccc34)cc2)cc1 ; c1ccc(-c2ccc(N(c3ccc(-c4cccc5c4oc4c5ccc5sc6ccccc6c54)cc3)c3cccc4c3sc3c(-c5ccccc5)cccc34)cc2)cc1 ; c1ccc(-c2ccc(N(c3ccc(-c4cccc5c4oc4c5ccc5sc6ccccc6c54)cc3)c3ccc(-c4cccc5c4sc4ccccc45)cc3)cc2)cc1 ; c1ccc(-c2ccc(N(c3ccc(-c4ccc5c(c4)oc4ccccc45)cc3)c3ccc(-c4cccc5c4oc4c5ccc5sc6ccccc6c54)cc3)cc2)cc1]

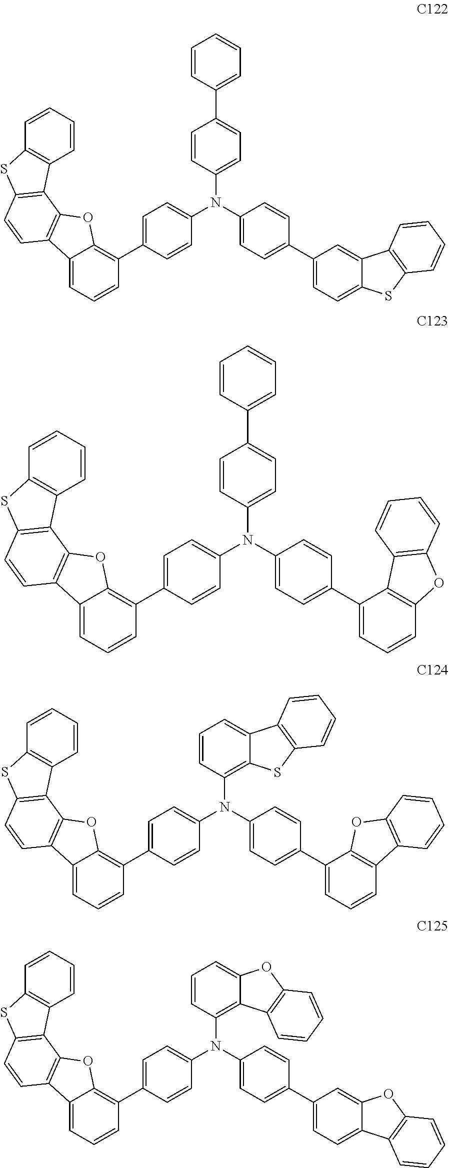 [SMILES: c1ccc(-c2ccc(N(c3ccc(-c4ccc5sc6ccccc6c5c4)cc3)c3ccc(-c4cccc5c4oc4c5ccc5sc6ccccc6c54)cc3)cc2)cc1 ; c1ccc(-c2ccc(N(c3ccc(-c4cccc5c4oc4c5ccc5sc6ccccc6c54)cc3)c3ccc(-c4cccc5oc6ccccc6c45)cc3)cc2)cc1 ; c1ccc2c(c1)oc1c(-c3ccc(N(c4ccc(-c5cccc6c5oc5c6ccc6sc7ccccc7c65)cc4)c4cccc5c4sc4ccccc45)cc3)cccc12 ; c1ccc2c(c1)oc1cc(-c3ccc(N(c4ccc(-c5cccc6c5oc5c6ccc6sc7ccccc7c65)cc4)c4cccc5oc6ccccc6c45)cc3)ccc12]

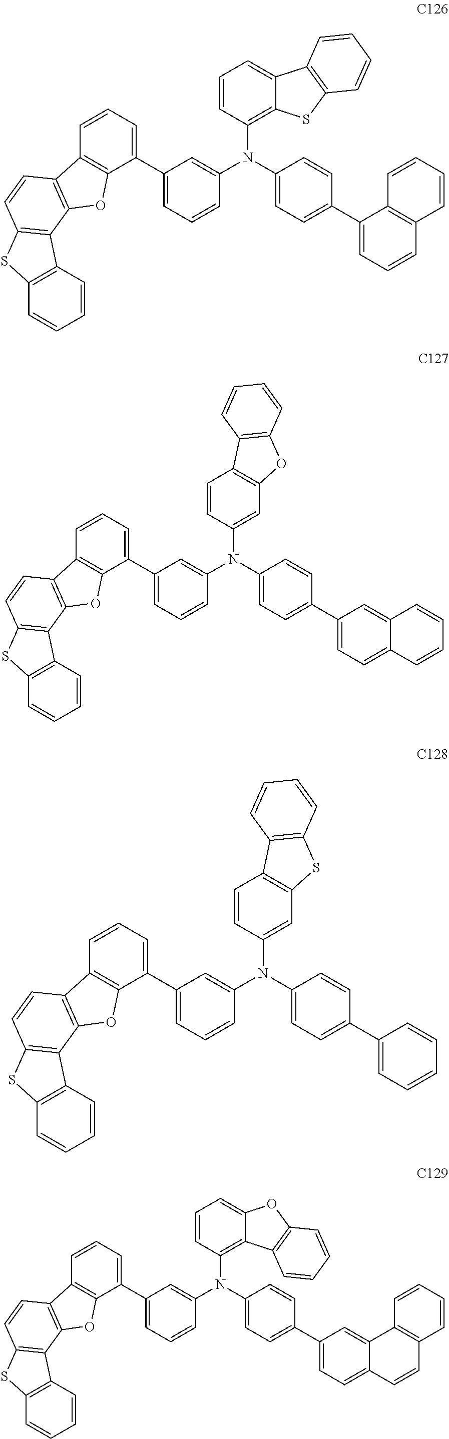 [SMILES: c1cc(-c2cccc3c2oc2c3ccc3sc4ccccc4c32)cc(N(c2ccc(-c3cccc4ccccc34)cc2)c2cccc3c2sc2ccccc23)c1 ; c1cc(-c2cccc3c2oc2c3ccc3sc4ccccc4c32)cc(N(c2ccc(-c3ccc4ccccc4c3)cc2)c2ccc3c(c2)oc2ccccc23)c1 ; c1ccc(-c2ccc(N(c3cccc(-c4cccc5c4oc4c5ccc5sc6ccccc6c54)c3)c3ccc4c(c3)sc3ccccc34)cc2)cc1 ; c1cc(-c2cccc3c2oc2c3ccc3sc4ccccc4c32)cc(N(c2ccc(-c3ccc4ccc5ccccc5c4c3)cc2)c2cccc3oc4ccccc4c23)c1]

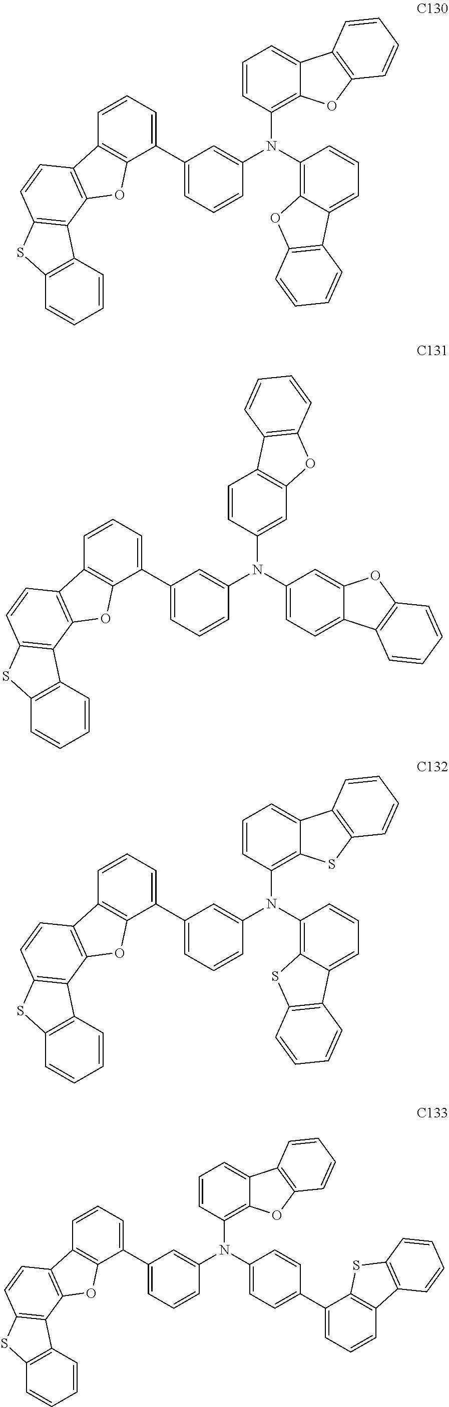 [SMILES: c1cc(-c2cccc3c2oc2c3ccc3sc4ccccc4c32)cc(N(c2cccc3c2oc2ccccc23)c2cccc3c2oc2ccccc23)c1 ; c1cc(-c2cccc3c2oc2c3ccc3sc4ccccc4c32)cc(N(c2ccc3c(c2)oc2ccccc23)c2ccc3c(c2)oc2ccccc23)c1 ; c1cc(-c2cccc3c2oc2c3ccc3sc4ccccc4c32)cc(N(c2cccc3c2sc2ccccc23)c2cccc3c2sc2ccccc23)c1 ; c1cc(-c2cccc3c2oc2c3ccc3sc4ccccc4c32)cc(N(c2ccc(-c3cccc4c3sc3ccccc34)cc2)c2cccc3c2oc2ccccc23)c1]

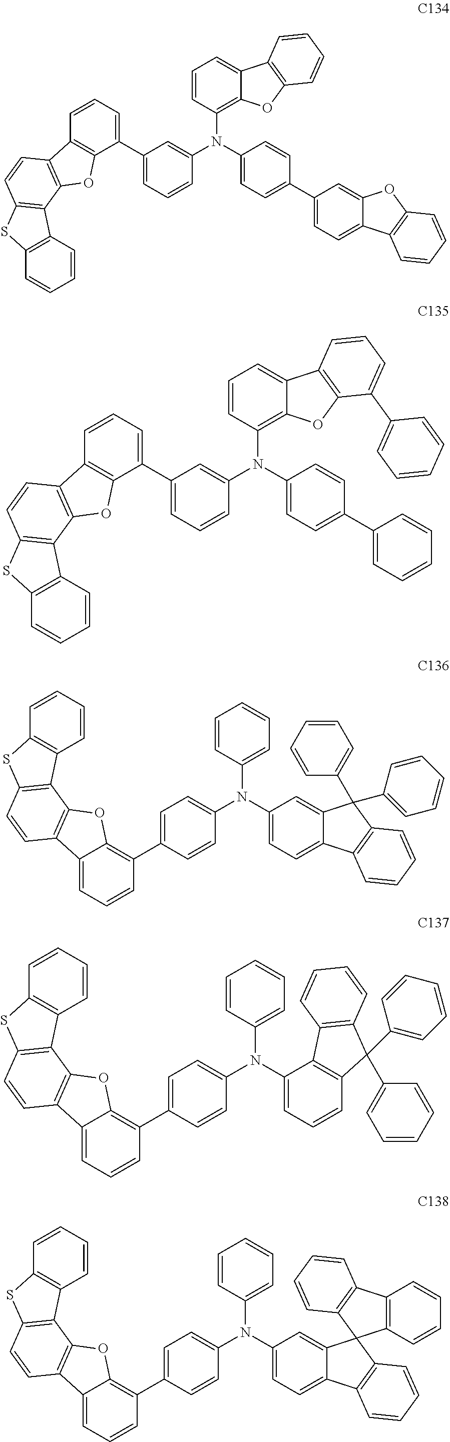 [SMILES: c1cc(-c2cccc3c2oc2c3ccc3sc4ccccc4c32)cc(N(c2ccc(-c3ccc4c(c3)oc3ccccc34)cc2)c2cccc3c2oc2ccccc23)c1 ; c1ccc(-c2ccc(N(c3cccc(-c4cccc5c4oc4c5ccc5sc6ccccc6c54)c3)c3cccc4c3oc3c(-c5ccccc5)cccc34)cc2)cc1 ; c1ccc(N(c2ccc(-c3cccc4c3oc3c4ccc4sc5ccccc5c43)cc2)c2ccc3c(c2)C(c2ccccc2)(c2ccccc2)c2ccccc2-3)cc1 ; c1ccc(N(c2ccc(-c3cccc4c3oc3c4ccc4sc5ccccc5c43)cc2)c2cccc3c2-c2ccccc2C3(c2ccccc2)c2ccccc2)cc1 ; c1ccc(N(c2ccc(-c3cccc4c3oc3c4ccc4sc5ccccc5c43)cc2)c2ccc3c(c2)C2(c4ccccc4-c4ccccc42)c2ccccc2-3)cc1]

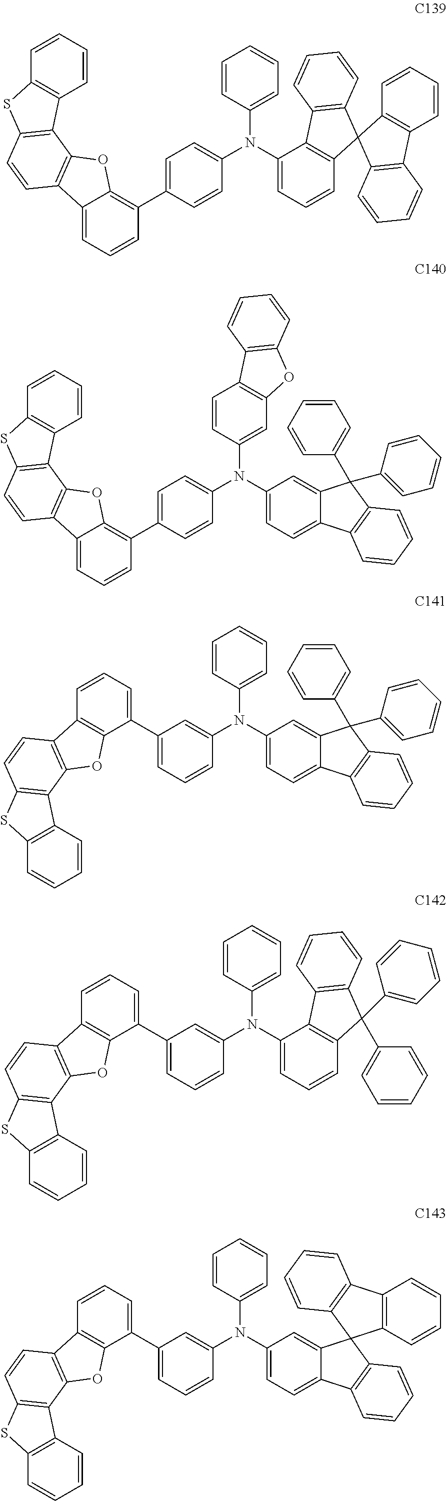 [SMILES: c1ccc(N(c2ccc(-c3cccc4c3oc3c4ccc4sc5ccccc5c43)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6c5oc5c6ccc6sc7ccccc7c65)cc4)c4ccc5c(c4)oc4ccccc45)cc32)cc1 ; c1ccc(N(c2cccc(-c3cccc4c3oc3c4ccc4sc5ccccc5c43)c2)c2ccc3c(c2)C(c2ccccc2)(c2ccccc2)c2ccccc2-3)cc1 ; c1ccc(N(c2cccc(-c3cccc4c3oc3c4ccc4sc5ccccc5c43)c2)c2cccc3c2-c2ccccc2C3(c2ccccc2)c2ccccc2)cc1 ; c1ccc(N(c2cccc(-c3cccc4c3oc3c4ccc4sc5ccccc5c43)c2)c2ccc3c(c2)C2(c4ccccc4-c4ccccc42)c2ccccc2-3)cc1]

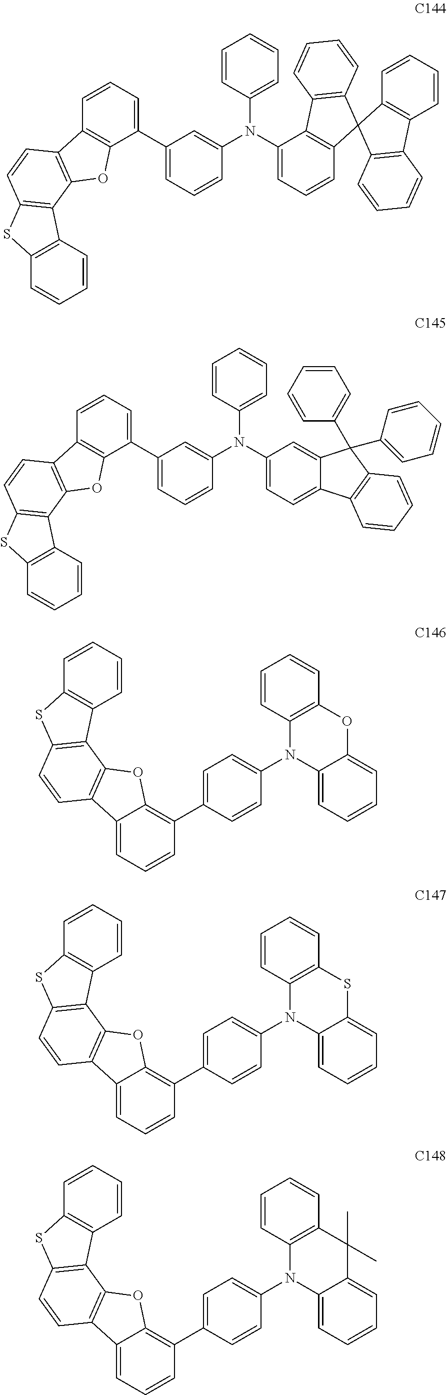 [SMILES: c1ccc(N(c2cccc(-c3cccc4c3oc3c4ccc4sc5ccccc5c43)c2)c2cccc3c2-c2ccccc2C32c3ccccc3-c3ccccc32)cc1 ; c1ccc(N(c2cccc(-c3cccc4c3oc3c4ccc4sc5ccccc5c43)c2)c2ccc3c(c2)C(c2ccccc2)(c2ccccc2)c2ccccc2-3)cc1 ; c1ccc2c(c1)Oc1ccccc1N2c1ccc(-c2cccc3c2oc2c3ccc3sc4ccccc4c32)cc1 ; c1ccc2c(c1)Sc1ccccc1N2c1ccc(-c2cccc3c2oc2c3ccc3sc4ccccc4c32)cc1 ; CC1(C)c2ccccc2N(c2ccc(-c3cccc4c3oc3c4ccc4sc5ccccc5c43)cc2)c2ccccc21]

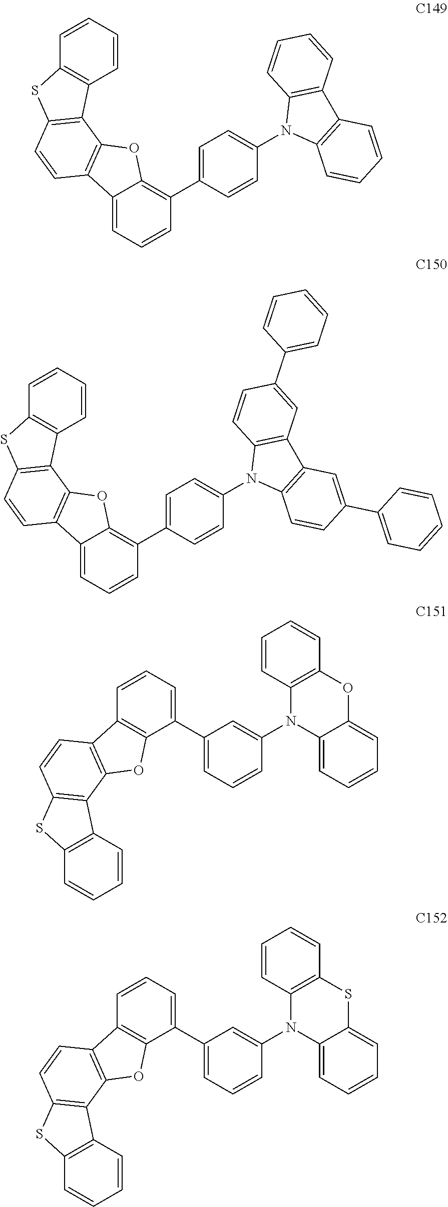 [SMILES: c1ccc2c(c1)sc1ccc3c4cccc(-c5ccc(-n6c7ccccc7c7ccccc76)cc5)c4oc3c12 ; c1ccc(-c2ccc3c(c2)c2cc(-c4ccccc4)ccc2n3-c2ccc(-c3cccc4c3oc3c4ccc4sc5ccccc5c43)cc2)cc1 ; c1cc(-c2cccc3c2oc2c3ccc3sc4ccccc4c32)cc(N2c3ccccc3Oc3ccccc32)c1 ; c1cc(-c2cccc3c2oc2c3ccc3sc4ccccc4c32)cc(N2c3ccccc3Sc3ccccc32)c1]

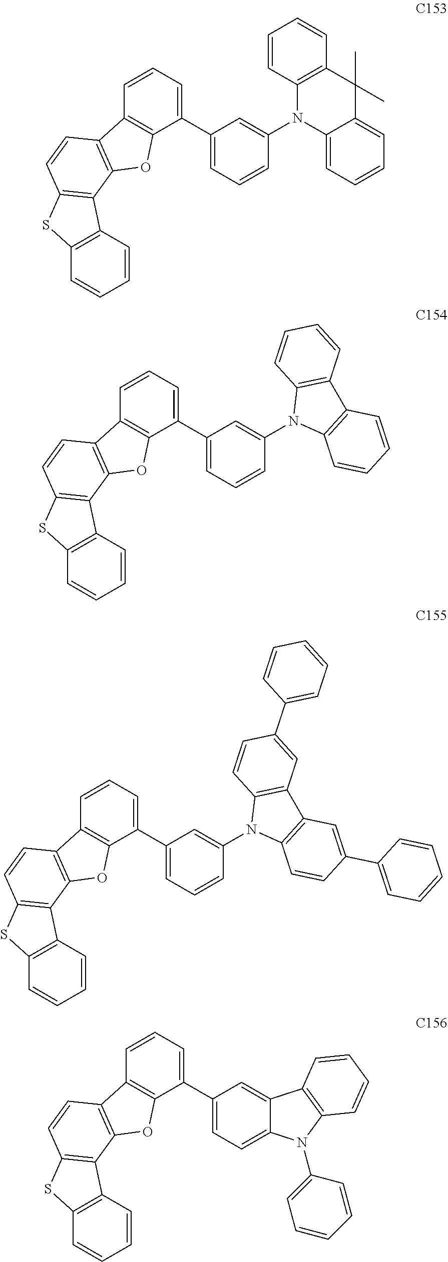 [SMILES: CC1(C)c2ccccc2N(c2cccc(-c3cccc4c3oc3c4ccc4sc5ccccc5c43)c2)c2ccccc21 ; c1cc(-c2cccc3c2oc2c3ccc3sc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3oc3c4ccc4sc5ccccc5c43)c2)cc1 ; c1ccc(-n2c3ccccc3c3cc(-c4cccc5c4oc4c5ccc5sc6ccccc6c54)ccc32)cc1]

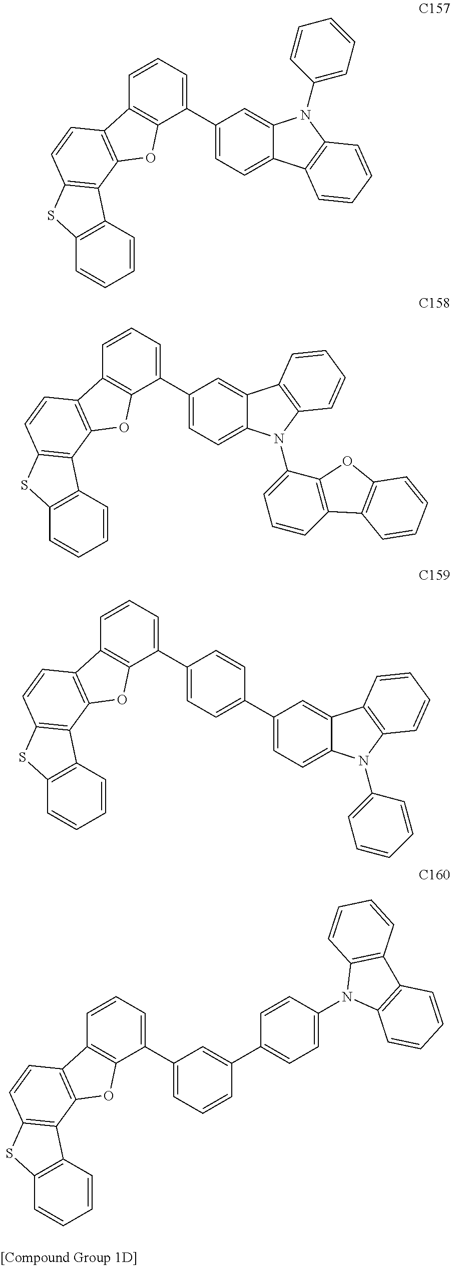

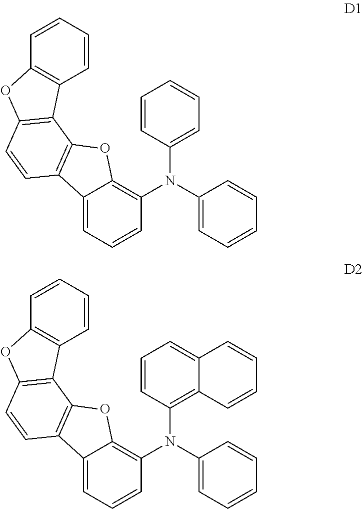 [SMILES: c1ccc(N(c2ccccc2)c2cccc3c2oc2c3ccc3oc4ccccc4c32)cc1 ; c1ccc(N(c2cccc3ccccc23)c2cccc3c2oc2c3ccc3oc4ccccc4c32)cc1]

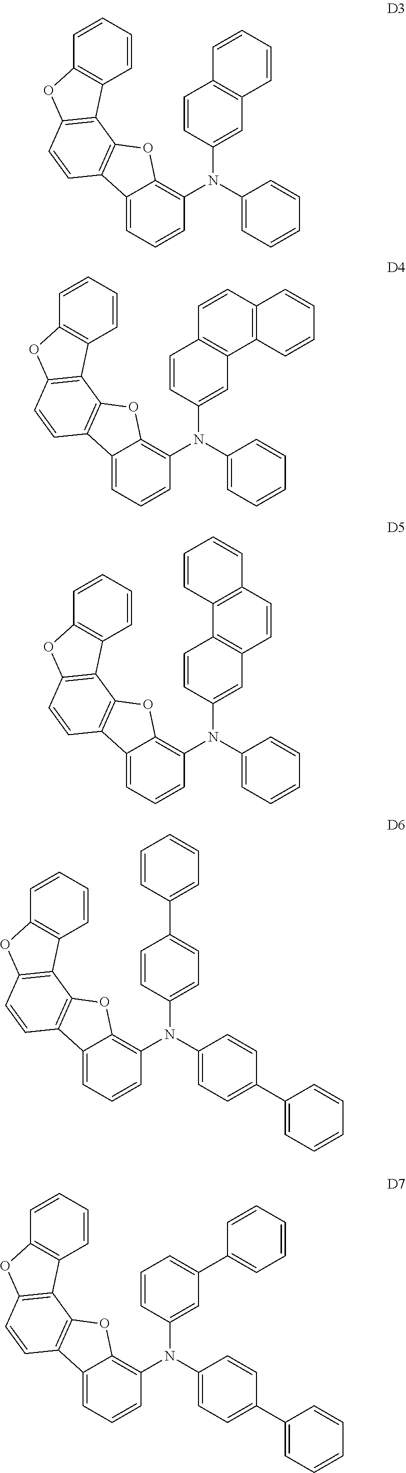

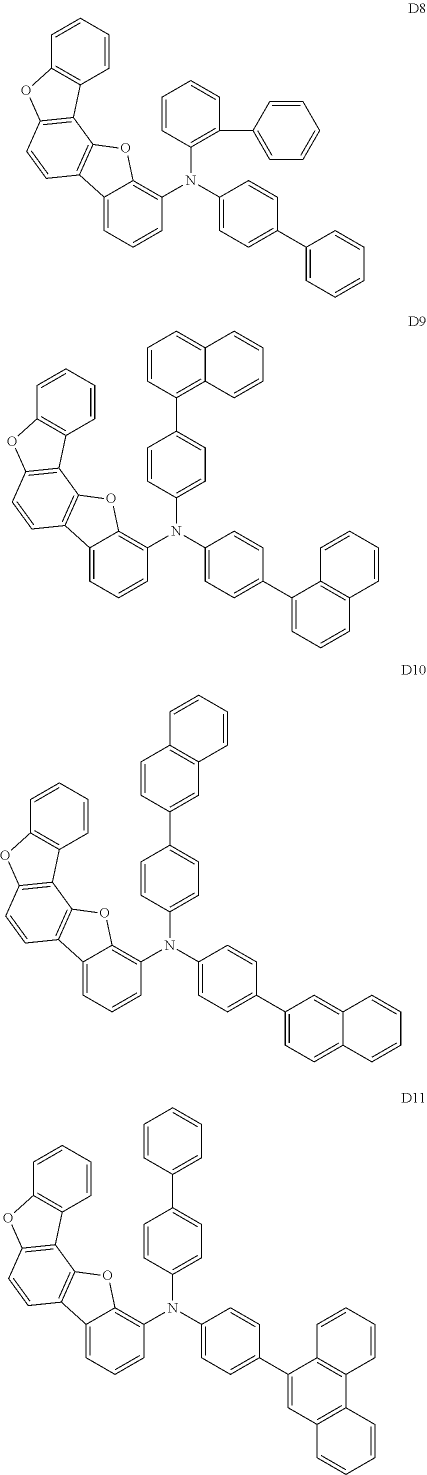 [SMILES: c1ccc(-c2ccc(N(c3ccccc3-c3ccccc3)c3cccc4c3oc3c4ccc4oc5ccccc5c43)cc2)cc1 ; c1ccc2c(-c3ccc(N(c4ccc(-c5cccc6ccccc56)cc4)c4cccc5c4oc4c5ccc5oc6ccccc6c54)cc3)cccc2c1 ; c1ccc2cc(-c3ccc(N(c4ccc(-c5ccc6ccccc6c5)cc4)c4cccc5c4oc4c5ccc5oc6ccccc6c54)cc3)ccc2c1 ; c1ccc(-c2ccc(N(c3ccc(-c4cc5ccccc5c5ccccc45)cc3)c3cccc4c3oc3c4ccc4oc5ccccc5c43)cc2)cc1]

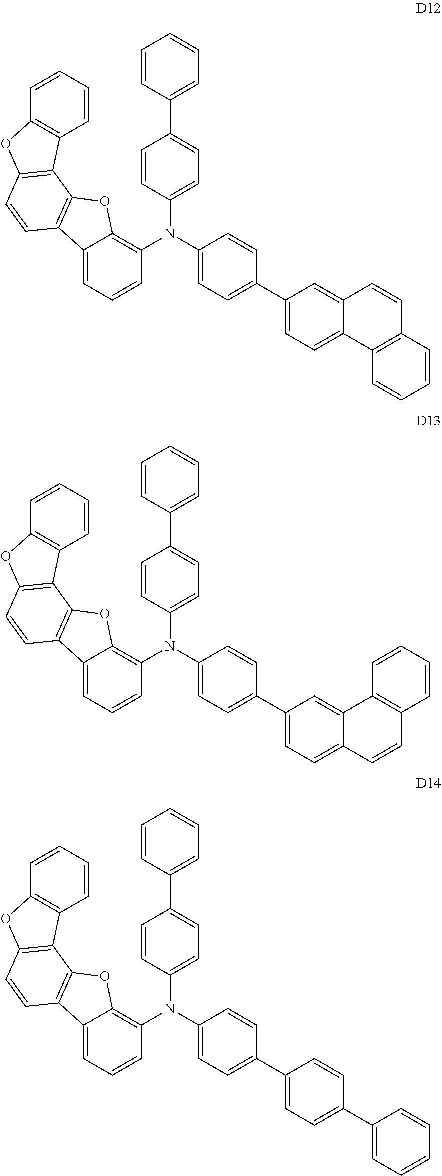 [SMILES: c1ccc(-c2ccc(N(c3ccc(-c4ccc5c(ccc6ccccc65)c4)cc3)c3cccc4c3oc3c4ccc4oc5ccccc5c43)cc2)cc1 ; c1ccc(-c2ccc(N(c3ccc(-c4ccc5ccc6ccccc6c5c4)cc3)c3cccc4c3oc3c4ccc4oc5ccccc5c43)cc2)cc1 ; c1ccc(-c2ccc(-c3ccc(N(c4ccc(-c5ccccc5)cc4)c4cccc5c4oc4c5ccc5oc6ccccc6c54)cc3)cc2)cc1]

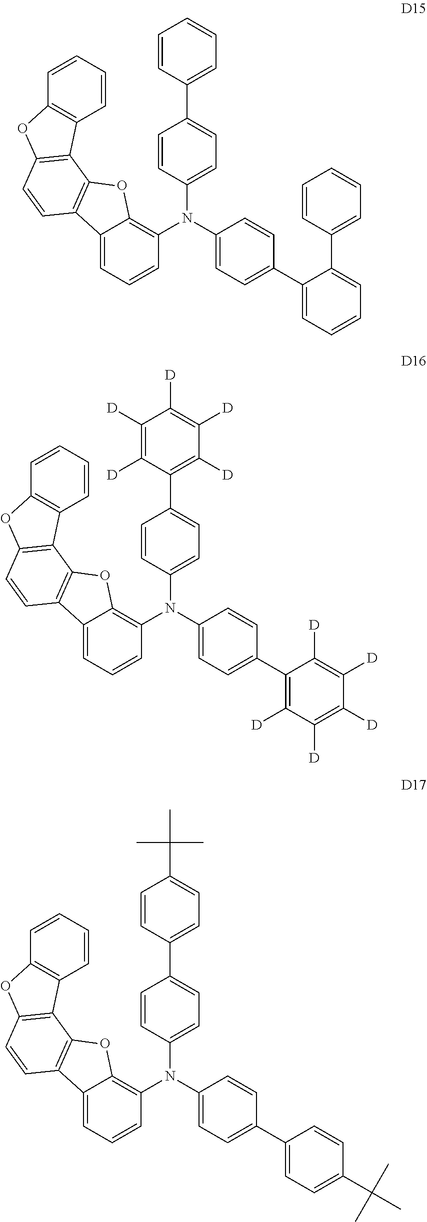 [SMILES: c1ccc(-c2ccc(N(c3ccc(-c4ccccc4-c4ccccc4)cc3)c3cccc4c3oc3c4ccc4oc5ccccc5c43)cc2)cc1 ; [2H]c1c([2H])c([2H])c(-c2ccc(N(c3ccc(-c4c([2H])c([2H])c([2H])c([2H])c4[2H])cc3)c3cccc4c3oc3c4ccc4oc5ccccc5c43)cc2)c([2H])c1[2H] ; CC(C)(C)c1ccc(-c2ccc(N(c3ccc(-c4ccc(C(C)(C)C)cc4)cc3)c3cccc4c3oc3c4ccc4oc5ccccc5c43)cc2)cc1]

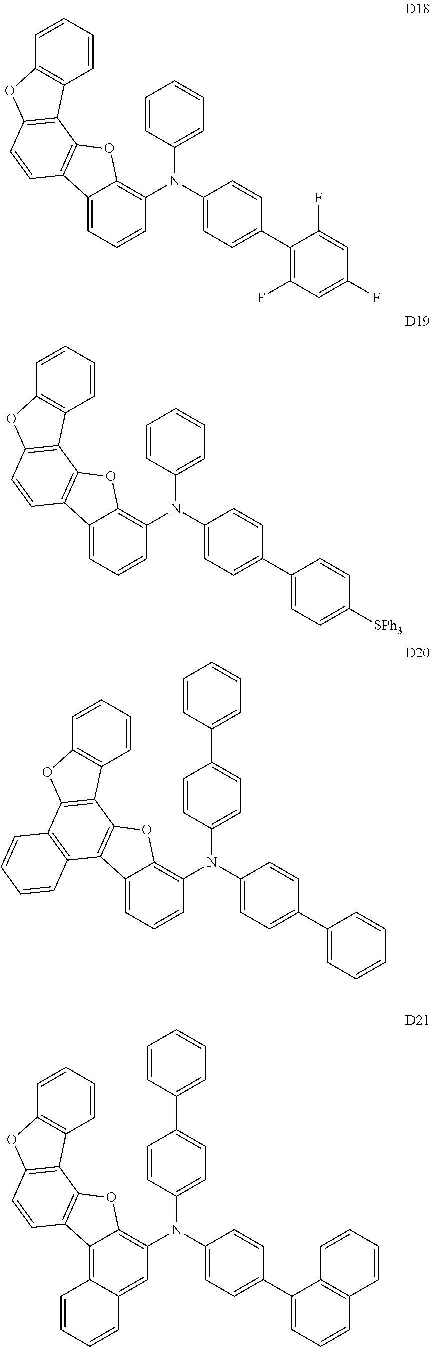

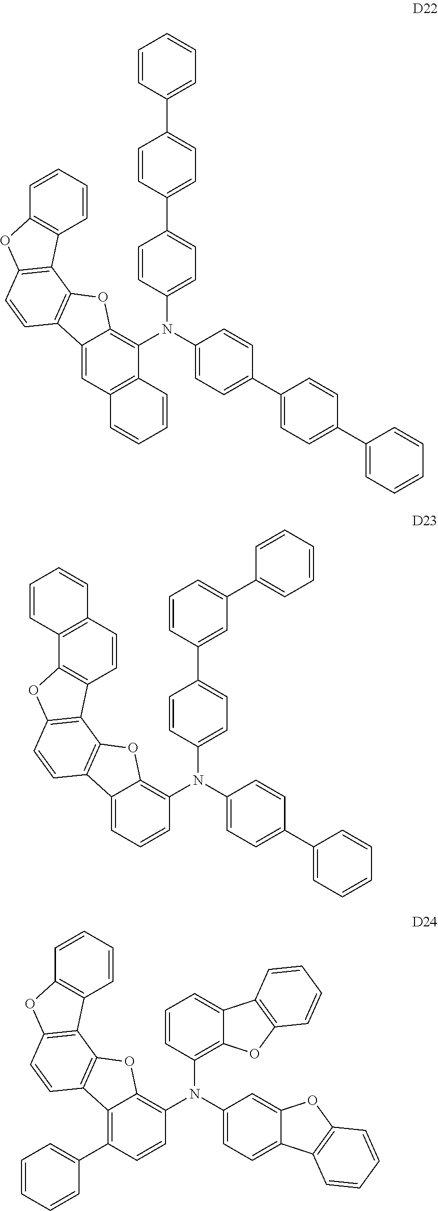 [SMILES: c1ccc(-c2ccc(-c3ccc(N(c4ccc(-c5ccc(-c6ccccc6)cc5)cc4)c4c5ccccc5cc5c4oc4c5ccc5oc6ccccc6c54)cc3)cc2)cc1 ; c1ccc(-c2ccc(N(c3ccc(-c4cccc(-c5ccccc5)c4)cc3)c3cccc4c3oc3c4ccc4oc5c6ccccc6ccc5c43)cc2)cc1 ; c1ccc(-c2ccc(N(c3ccc4c(c3)oc3ccccc34)c3cccc4c3oc3ccccc34)c3oc4c(ccc5oc6ccccc6c54)c23)cc1]

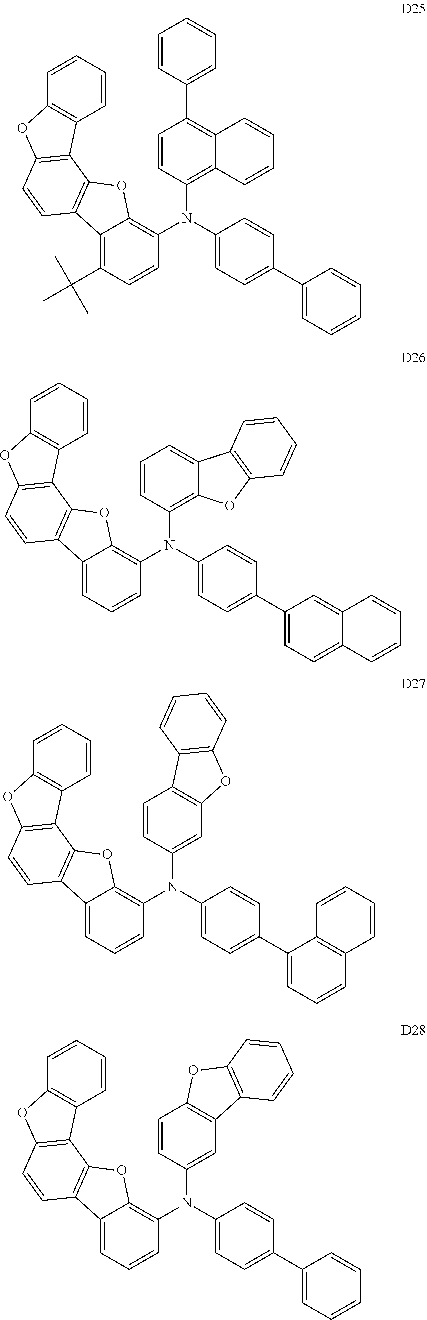 [SMILES: CC(C)(C)c1ccc(N(c2ccc(-c3ccccc3)cc2)c2ccc(-c3ccccc3)c3ccccc23)c2oc3c(ccc4oc5ccccc5c43)c12 ; c1ccc2cc(-c3ccc(N(c4cccc5c4oc4ccccc45)c4cccc5c4oc4c5ccc5oc6ccccc6c54)cc3)ccc2c1 ; c1ccc2c(-c3ccc(N(c4ccc5c(c4)oc4ccccc45)c4cccc5c4oc4c5ccc5oc6ccccc6c54)cc3)cccc2c1 ; c1ccc(-c2ccc(N(c3ccc4oc5ccccc5c4c3)c3cccc4c3oc3c4ccc4oc5ccccc5c43)cc2)cc1]

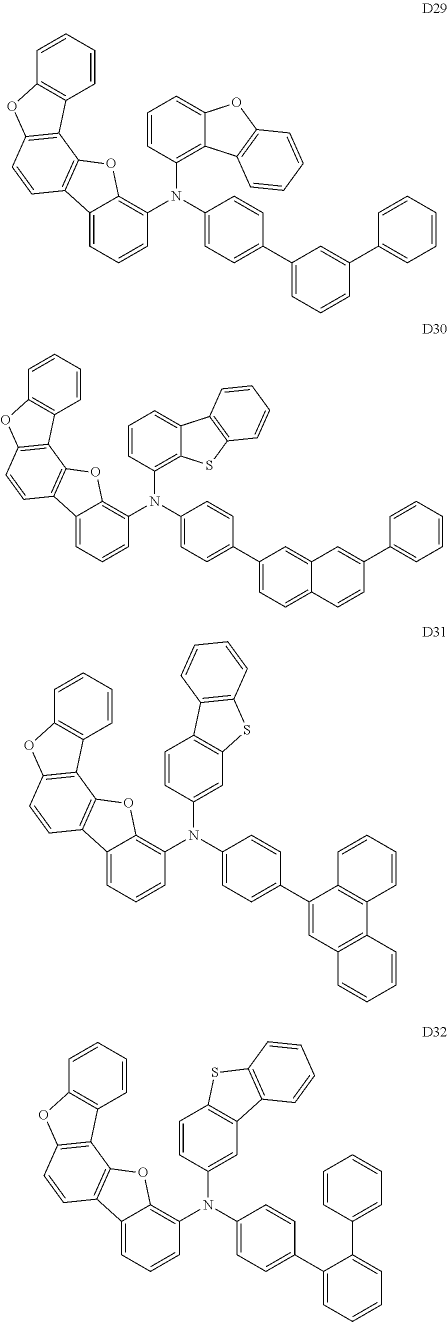 [SMILES: c1ccc(-c2cccc(-c3ccc(N(c4cccc5c4oc4c5ccc5oc6ccccc6c54)c4cccc5oc6ccccc6c45)cc3)c2)cc1 ; c1ccc(-c2ccc3ccc(-c4ccc(N(c5cccc6c5oc5c6ccc6oc7ccccc7c65)c5cccc6c5sc5ccccc56)cc4)cc3c2)cc1 ; c1ccc2c(c1)cc(-c1ccc(N(c3ccc4c(c3)sc3ccccc34)c3cccc4c3oc3c4ccc4oc5ccccc5c43)cc1)c1ccccc12 ; c1ccc(-c2ccccc2-c2ccc(N(c3ccc4sc5ccccc5c4c3)c3cccc4c3oc3c4ccc4oc5ccccc5c43)cc2)cc1]

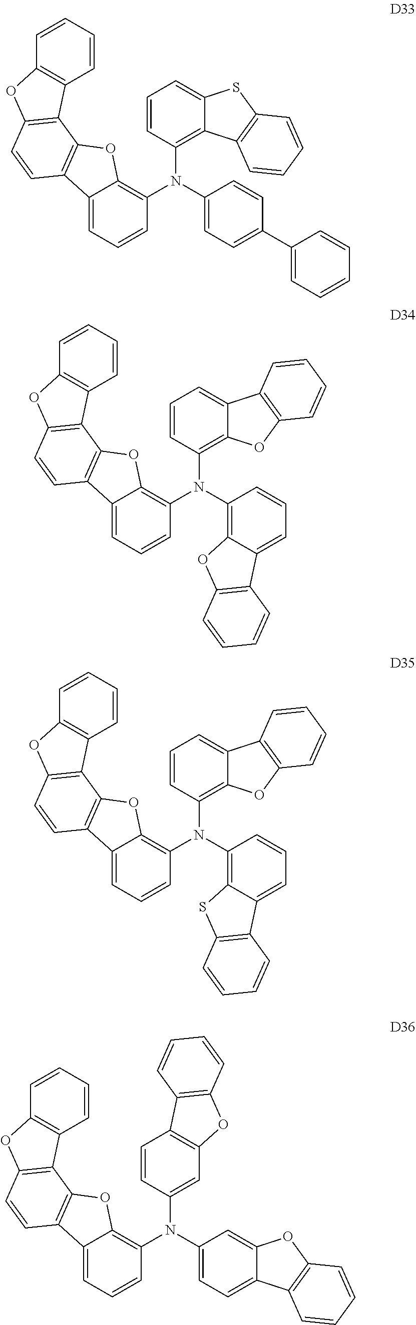

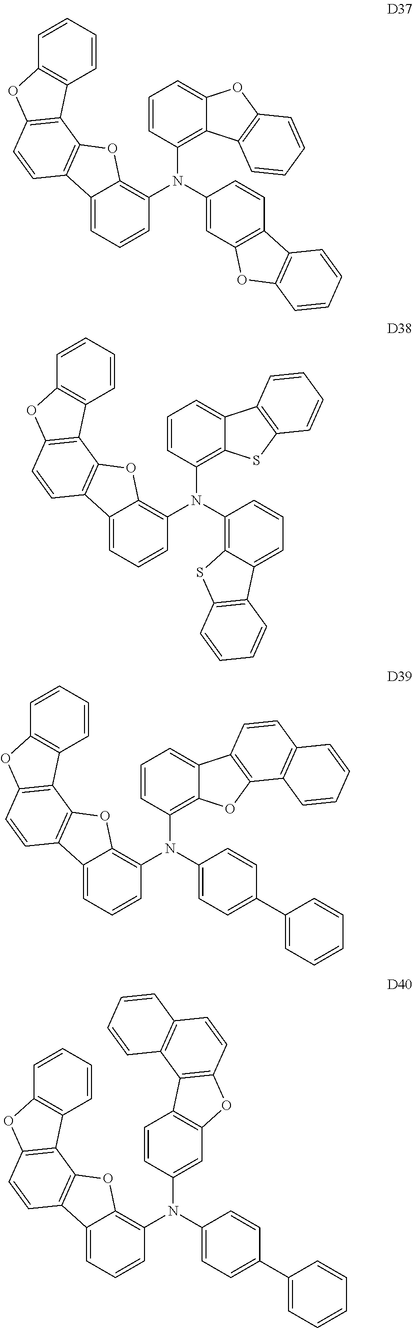 [SMILES: c1ccc2c(c1)oc1cc(N(c3cccc4c3oc3c4ccc4oc5ccccc5c43)c3cccc4oc5ccccc5c34)ccc12 ; c1ccc2c(c1)oc1ccc3c4cccc(N(c5cccc6c5sc5ccccc56)c5cccc6c5sc5ccccc56)c4oc3c12 ; c1ccc(-c2ccc(N(c3cccc4c3oc3c5ccccc5ccc43)c3cccc4c3oc3c4ccc4oc5ccccc5c43)cc2)cc1 ; c1ccc(-c2ccc(N(c3ccc4c(c3)oc3ccc5ccccc5c34)c3cccc4c3oc3c4ccc4oc5ccccc5c43)cc2)cc1]

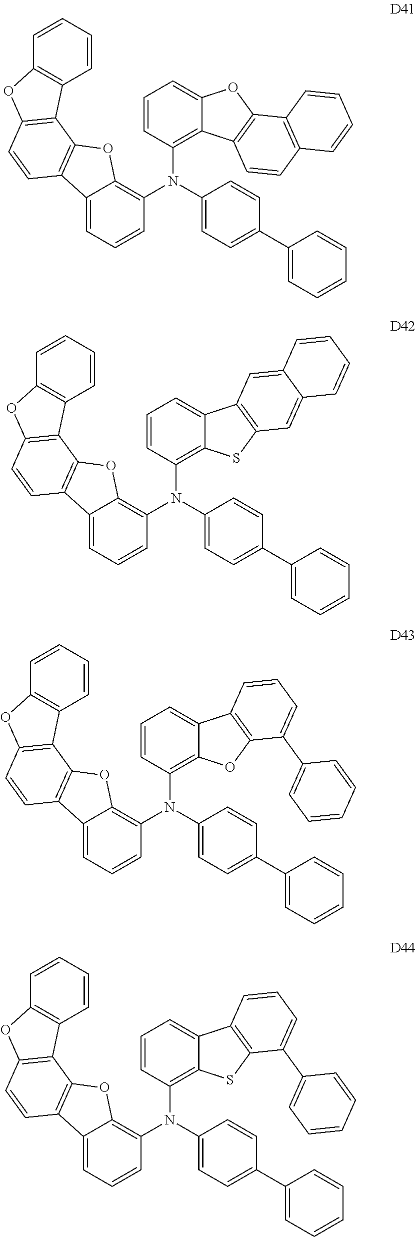

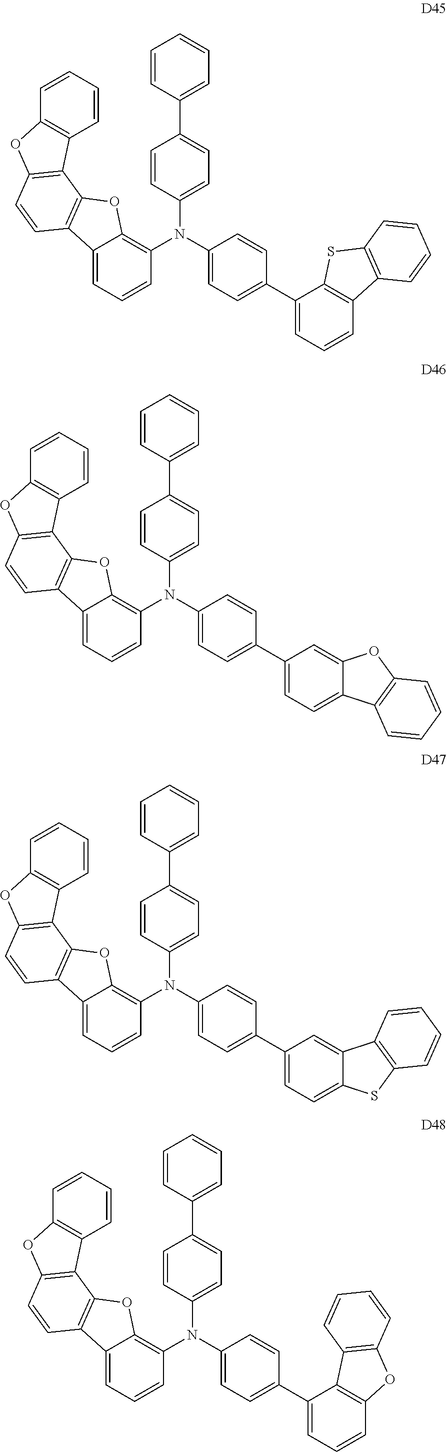 [SMILES: c1ccc(-c2ccc(N(c3ccc(-c4cccc5c4sc4ccccc45)cc3)c3cccc4c3oc3c4ccc4oc5ccccc5c43)cc2)cc1 ; c1ccc(-c2ccc(N(c3ccc(-c4ccc5c(c4)oc4ccccc45)cc3)c3cccc4c3oc3c4ccc4oc5ccccc5c43)cc2)cc1 ; c1ccc(-c2ccc(N(c3ccc(-c4ccc5sc6ccccc6c5c4)cc3)c3cccc4c3oc3c4ccc4oc5ccccc5c43)cc2)cc1 ; c1ccc(-c2ccc(N(c3ccc(-c4cccc5oc6ccccc6c45)cc3)c3cccc4c3oc3c4ccc4oc5ccccc5c43)cc2)cc1]

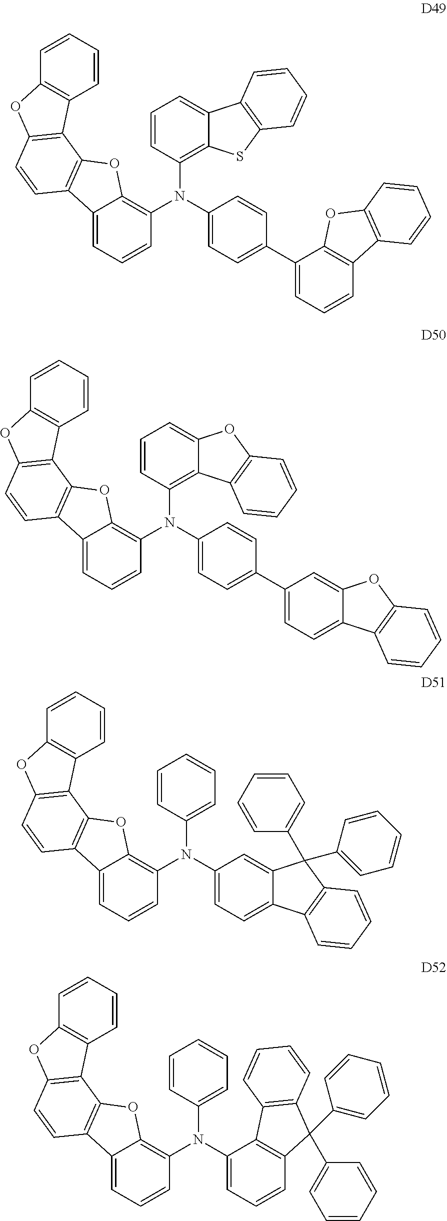 [SMILES: c1ccc2c(c1)oc1c(-c3ccc(N(c4cccc5c4oc4c5ccc5oc6ccccc6c54)c4cccc5c4sc4ccccc45)cc3)cccc12 ; c1ccc2c(c1)oc1cc(-c3ccc(N(c4cccc5c4oc4c5ccc5oc6ccccc6c54)c4cccc5oc6ccccc6c45)cc3)ccc12 ; c1ccc(N(c2ccc3c(c2)C(c2ccccc2)(c2ccccc2)c2ccccc2-3)c2cccc3c2oc2c3ccc3oc4ccccc4c32)cc1 ; c1ccc(N(c2cccc3c2-c2ccccc2C3(c2ccccc2)c2ccccc2)c2cccc3c2oc2c3ccc3oc4ccccc4c32)cc1]

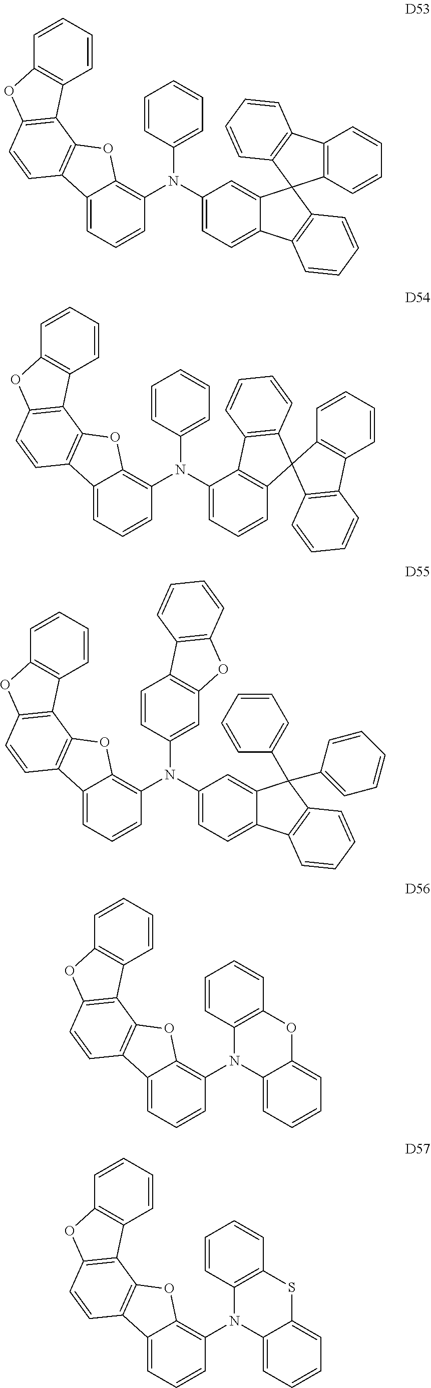 [SMILES: c1ccc(N(c2ccc3c(c2)C2(c4ccccc4-c4ccccc42)c2ccccc2-3)c2cccc3c2oc2c3ccc3oc4ccccc4c32)cc1 ; c1ccc(N(c2cccc3c2-c2ccccc2C32c3ccccc3-c3ccccc32)c2cccc3c2oc2c3ccc3oc4ccccc4c32)cc1 ; c1ccc(C2(c3ccccc3)c3ccccc3-c3ccc(N(c4ccc5c(c4)oc4ccccc45)c4cccc5c4oc4c5ccc5oc6ccccc6c54)cc32)cc1 ; c1ccc2c(c1)Oc1ccccc1N2c1cccc2c1oc1c2ccc2oc3ccccc3c21 ; c1ccc2c(c1)Sc1ccccc1N2c1cccc2c1oc1c2ccc2oc3ccccc3c21]

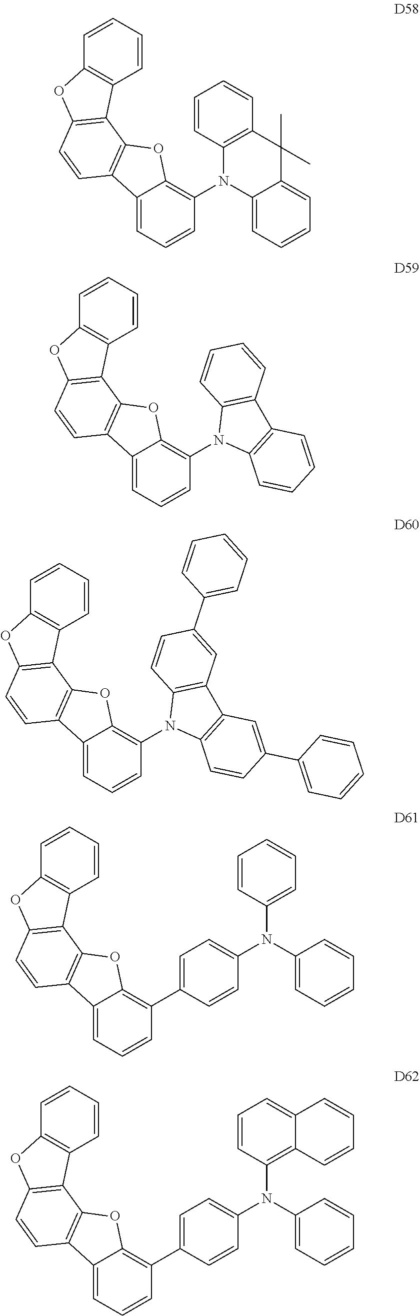

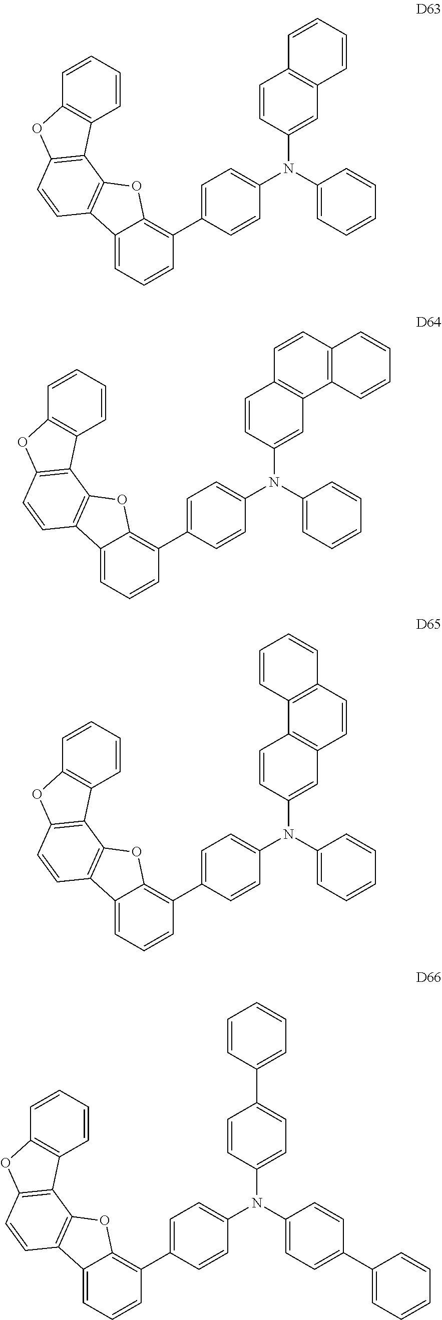 [SMILES: c1ccc(N(c2ccc(-c3cccc4c3oc3c4ccc4oc5ccccc5c43)cc2)c2ccc3ccccc3c2)cc1 ; c1ccc(N(c2ccc(-c3cccc4c3oc3c4ccc4oc5ccccc5c43)cc2)c2ccc3ccc4ccccc4c3c2)cc1 ; c1ccc(N(c2ccc(-c3cccc4c3oc3c4ccc4oc5ccccc5c43)cc2)c2ccc3c(ccc4ccccc43)c2)cc1 ; c1ccc(-c2ccc(N(c3ccc(-c4ccccc4)cc3)c3ccc(-c4cccc5c4oc4c5ccc5oc6ccccc6c54)cc3)cc2)cc1]

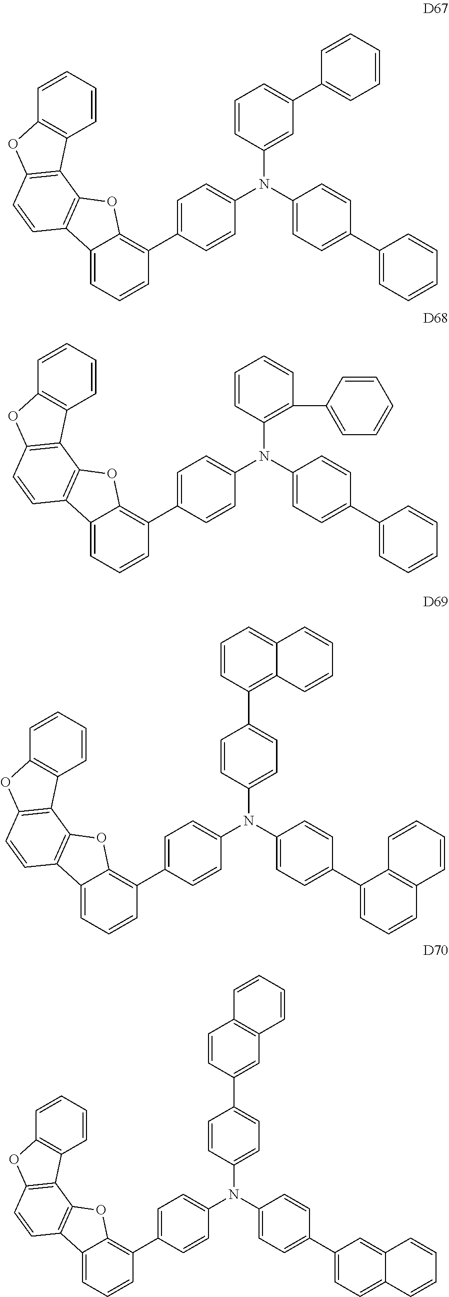 [SMILES: c1ccc(-c2ccc(N(c3ccc(-c4cccc5c4oc4c5ccc5oc6ccccc6c54)cc3)c3cccc(-c4ccccc4)c3)cc2)cc1 ; c1ccc(-c2ccc(N(c3ccc(-c4cccc5c4oc4c5ccc5oc6ccccc6c54)cc3)c3ccccc3-c3ccccc3)cc2)cc1 ; c1ccc2c(-c3ccc(N(c4ccc(-c5cccc6ccccc56)cc4)c4ccc(-c5cccc6c5oc5c6ccc6oc7ccccc7c65)cc4)cc3)cccc2c1 ; c1ccc2cc(-c3ccc(N(c4ccc(-c5ccc6ccccc6c5)cc4)c4ccc(-c5cccc6c5oc5c6ccc6oc7ccccc7c65)cc4)cc3)ccc2c1]

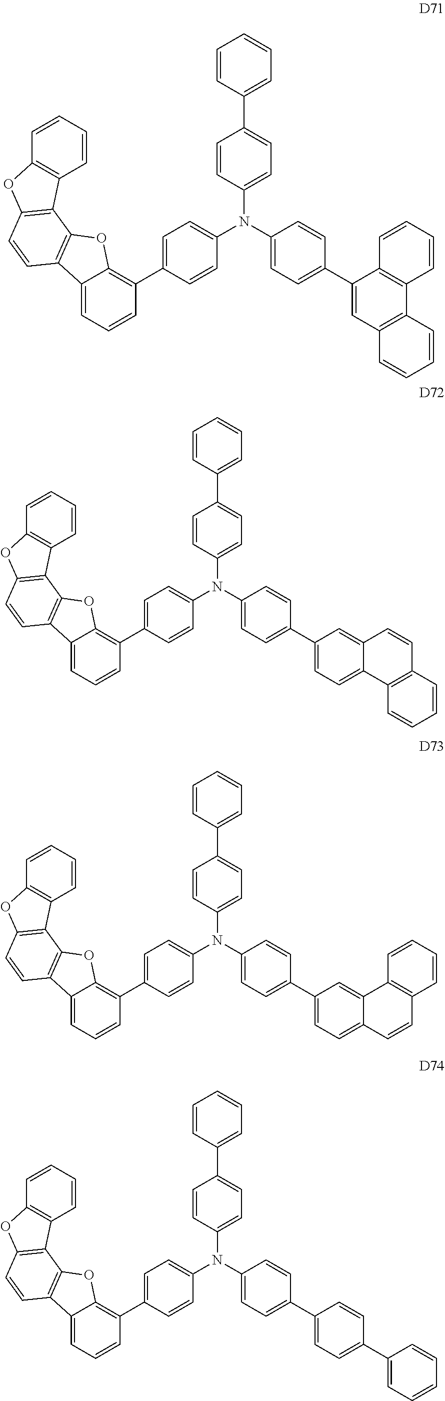 [SMILES: c1ccc(-c2ccc(N(c3ccc(-c4cc5ccccc5c5ccccc45)cc3)c3ccc(-c4cccc5c4oc4c5ccc5oc6ccccc6c54)cc3)cc2)cc1 ; c1ccc(-c2ccc(N(c3ccc(-c4ccc5c(ccc6ccccc65)c4)cc3)c3ccc(-c4cccc5c4oc4c5ccc5oc6ccccc6c54)cc3)cc2)cc1 ; c1ccc(-c2ccc(N(c3ccc(-c4ccc5ccc6ccccc6c5c4)cc3)c3ccc(-c4cccc5c4oc4c5ccc5oc6ccccc6c54)cc3)cc2)cc1 ; c1ccc(-c2ccc(-c3ccc(N(c4ccc(-c5ccccc5)cc4)c4ccc(-c5cccc6c5oc5c6ccc6oc7ccccc7c65)cc4)cc3)cc2)cc1]

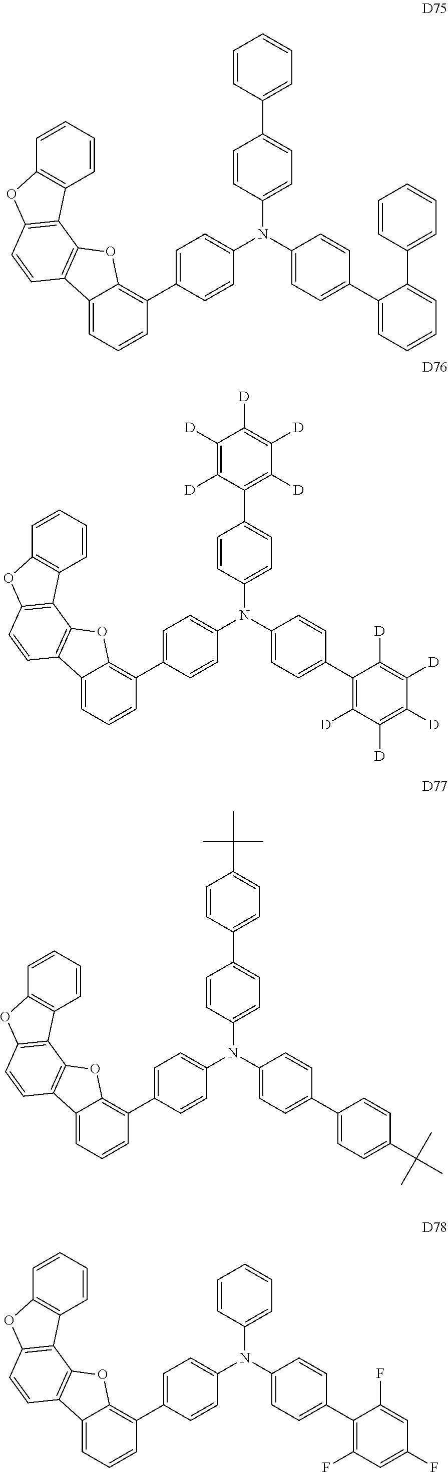 [SMILES: c1ccc(-c2ccc(N(c3ccc(-c4ccccc4-c4ccccc4)cc3)c3ccc(-c4cccc5c4oc4c5ccc5oc6ccccc6c54)cc3)cc2)cc1 ; [2H]c1c([2H])c([2H])c(-c2ccc(N(c3ccc(-c4c([2H])c([2H])c([2H])c([2H])c4[2H])cc3)c3ccc(-c4cccc5c4oc4c5ccc5oc6ccccc6c54)cc3)cc2)c([2H])c1[2H] ; CC(C)(C)c1ccc(-c2ccc(N(c3ccc(-c4ccc(C(C)(C)C)cc4)cc3)c3ccc(-c4cccc5c4oc4c5ccc5oc6ccccc6c54)cc3)cc2)cc1 ; Fc1cc(F)c(-c2ccc(N(c3ccccc3)c3ccc(-c4cccc5c4oc4c5ccc5oc6ccccc6c54)cc3)cc2)c(F)c1]

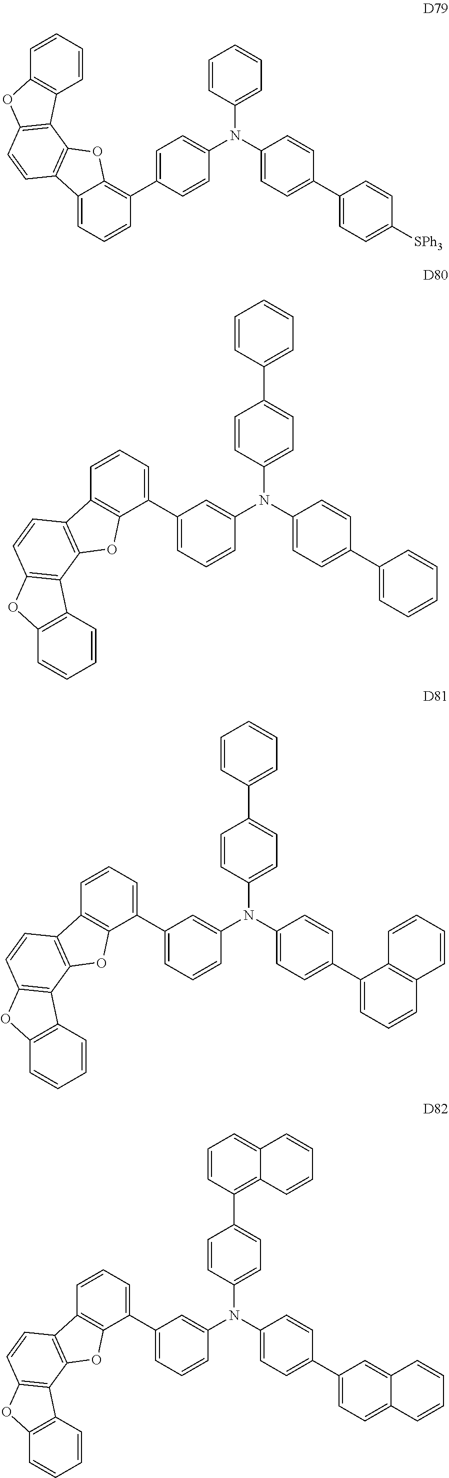

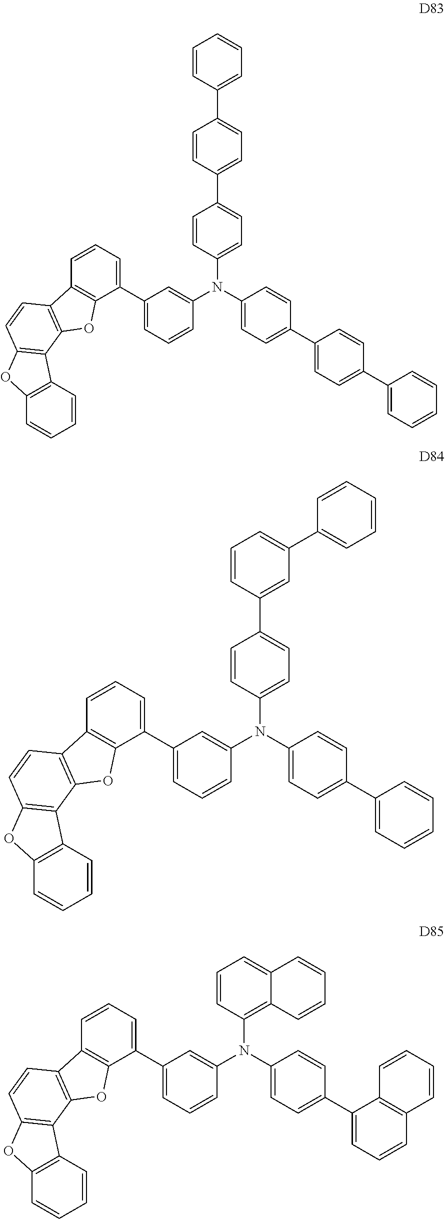 [SMILES: c1ccc(-c2ccc(-c3ccc(N(c4ccc(-c5ccc(-c6ccccc6)cc5)cc4)c4cccc(-c5cccc6c5oc5c6ccc6oc7ccccc7c65)c4)cc3)cc2)cc1 ; c1ccc(-c2ccc(N(c3ccc(-c4cccc(-c5ccccc5)c4)cc3)c3cccc(-c4cccc5c4oc4c5ccc5oc6ccccc6c54)c3)cc2)cc1 ; c1cc(-c2cccc3c2oc2c3ccc3oc4ccccc4c32)cc(N(c2ccc(-c3cccc4ccccc34)cc2)c2cccc3ccccc23)c1]

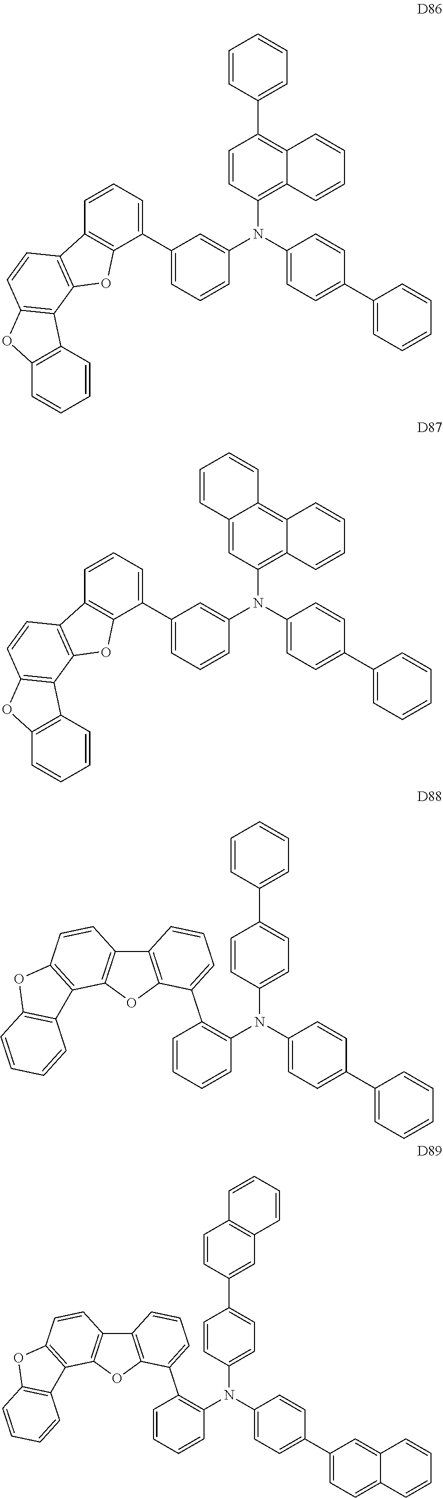 [SMILES: c1ccc(-c2ccc(N(c3cccc(-c4cccc5c4oc4c5ccc5oc6ccccc6c54)c3)c3ccc(-c4ccccc4)c4ccccc34)cc2)cc1 ; c1ccc(-c2ccc(N(c3cccc(-c4cccc5c4oc4c5ccc5oc6ccccc6c54)c3)c3cc4ccccc4c4ccccc34)cc2)cc1 ; c1ccc(-c2ccc(N(c3ccc(-c4ccccc4)cc3)c3ccccc3-c3cccc4c3oc3c4ccc4oc5ccccc5c43)cc2)cc1 ; c1ccc(N(c2ccc(-c3ccc4ccccc4c3)cc2)c2ccc(-c3ccc4ccccc4c3)cc2)c(-c2cccc3c2oc2c3ccc3oc4ccccc4c32)c1]

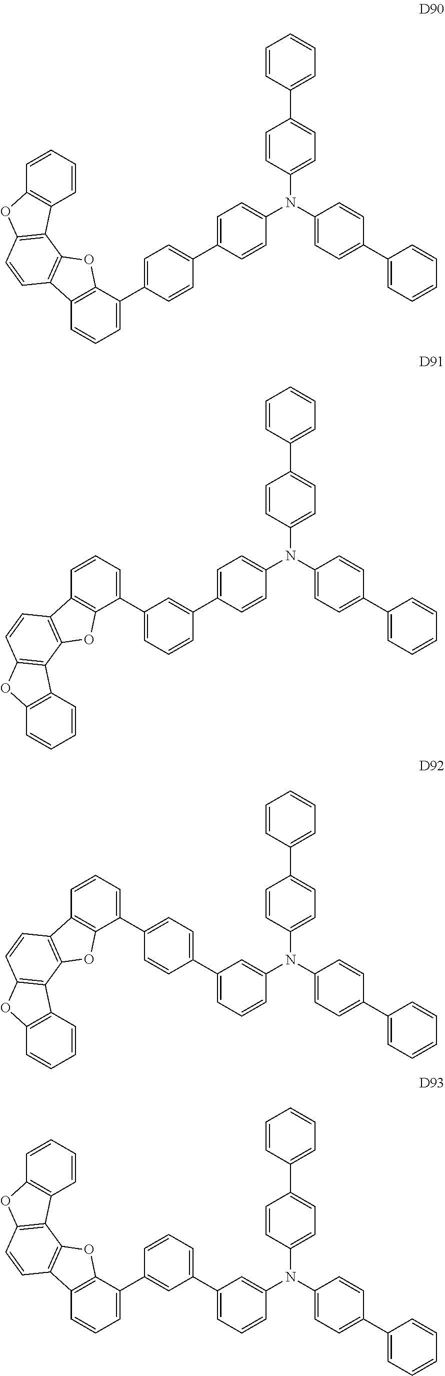 [SMILES: c1ccc(-c2ccc(N(c3ccc(-c4ccccc4)cc3)c3ccc(-c4ccc(-c5cccc6c5oc5c6ccc6oc7ccccc7c65)cc4)cc3)cc2)cc1 ; c1ccc(-c2ccc(N(c3ccc(-c4ccccc4)cc3)c3ccc(-c4cccc(-c5cccc6c5oc5c6ccc6oc7ccccc7c65)c4)cc3)cc2)cc1 ; c1ccc(-c2ccc(N(c3ccc(-c4ccccc4)cc3)c3cccc(-c4ccc(-c5cccc6c5oc5c6ccc6oc7ccccc7c65)cc4)c3)cc2)cc1 ; c1ccc(-c2ccc(N(c3ccc(-c4ccccc4)cc3)c3cccc(-c4cccc(-c5cccc6c5oc5c6ccc6oc7ccccc7c65)c4)c3)cc2)cc1]

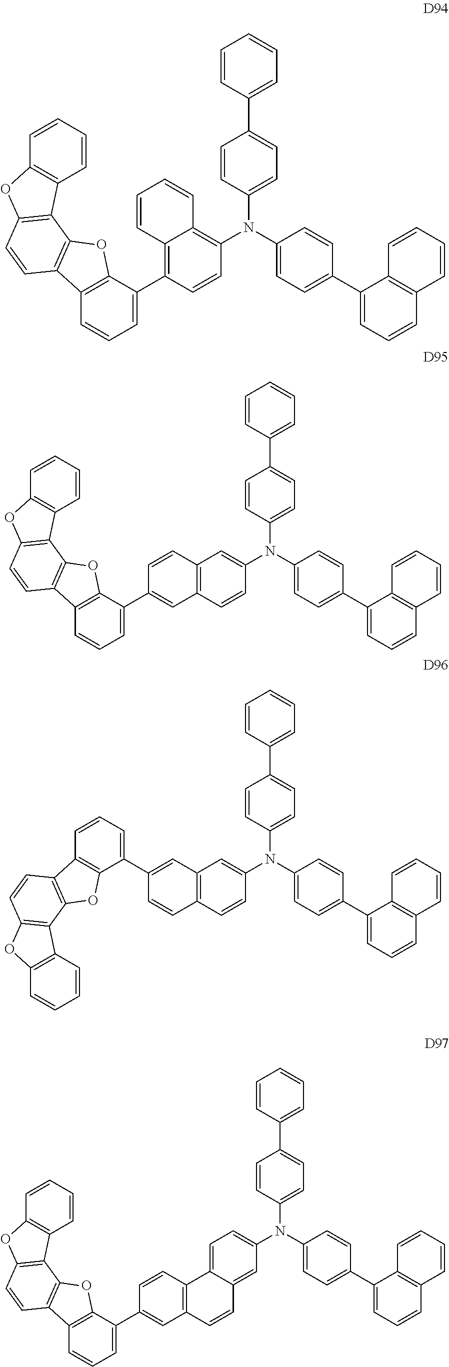 [SMILES: c1ccc(-c2ccc(N(c3ccc(-c4cccc5ccccc45)cc3)c3ccc(-c4cccc5c4oc4c5ccc5oc6ccccc6c54)c4ccccc34)cc2)cc1 ; c1ccc(-c2ccc(N(c3ccc(-c4cccc5ccccc45)cc3)c3ccc4cc(-c5cccc6c5oc5c6ccc6oc7ccccc7c65)ccc4c3)cc2)cc1 ; c1ccc(-c2ccc(N(c3ccc(-c4cccc5ccccc45)cc3)c3ccc4ccc(-c5cccc6c5oc5c6ccc6oc7ccccc7c65)cc4c3)cc2)cc1 ; c1ccc(-c2ccc(N(c3ccc(-c4cccc5ccccc45)cc3)c3ccc4c(ccc5cc(-c6cccc7c6oc6c7ccc7oc8ccccc8c76)ccc54)c3)cc2)cc1]

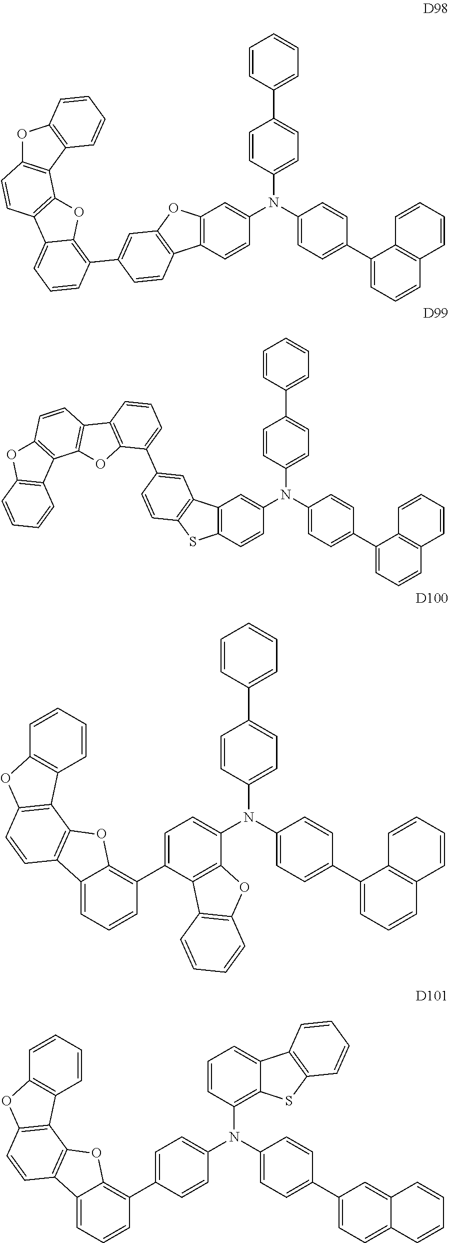 [SMILES: c1ccc(-c2ccc(N(c3ccc(-c4cccc5ccccc45)cc3)c3ccc4c(c3)oc3cc(-c5cccc6c5oc5c6ccc6oc7ccccc7c65)ccc34)cc2)cc1 ; c1ccc(-c2ccc(N(c3ccc(-c4cccc5ccccc45)cc3)c3ccc4sc5ccc(-c6cccc7c6oc6c7ccc7oc8ccccc8c76)cc5c4c3)cc2)cc1 ; c1ccc(-c2ccc(N(c3ccc(-c4cccc5ccccc45)cc3)c3ccc(-c4cccc5c4oc4c5ccc5oc6ccccc6c54)c4c3oc3ccccc34)cc2)cc1 ; c1ccc2cc(-c3ccc(N(c4ccc(-c5cccc6c5oc5c6ccc6oc7ccccc7c65)cc4)c4cccc5c4sc4ccccc45)cc3)ccc2c1]

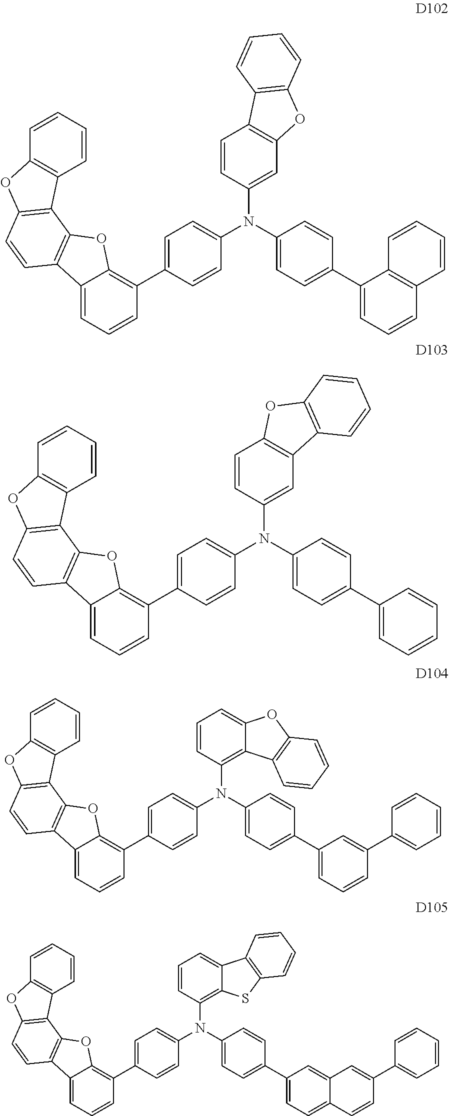 [SMILES: c1ccc2c(-c3ccc(N(c4ccc(-c5cccc6c5oc5c6ccc6oc7ccccc7c65)cc4)c4ccc5c(c4)oc4ccccc45)cc3)cccc2c1 ; c1ccc(-c2ccc(N(c3ccc(-c4cccc5c4oc4c5ccc5oc6ccccc6c54)cc3)c3ccc4oc5ccccc5c4c3)cc2)cc1 ; c1ccc(-c2cccc(-c3ccc(N(c4ccc(-c5cccc6c5oc5c6ccc6oc7ccccc7c65)cc4)c4cccc5oc6ccccc6c45)cc3)c2)cc1 ; c1ccc(-c2ccc3ccc(-c4ccc(N(c5ccc(-c6cccc7c6oc6c7ccc7oc8ccccc8c76)cc5)c5cccc6c5sc5ccccc56)cc4)cc3c2)cc1]

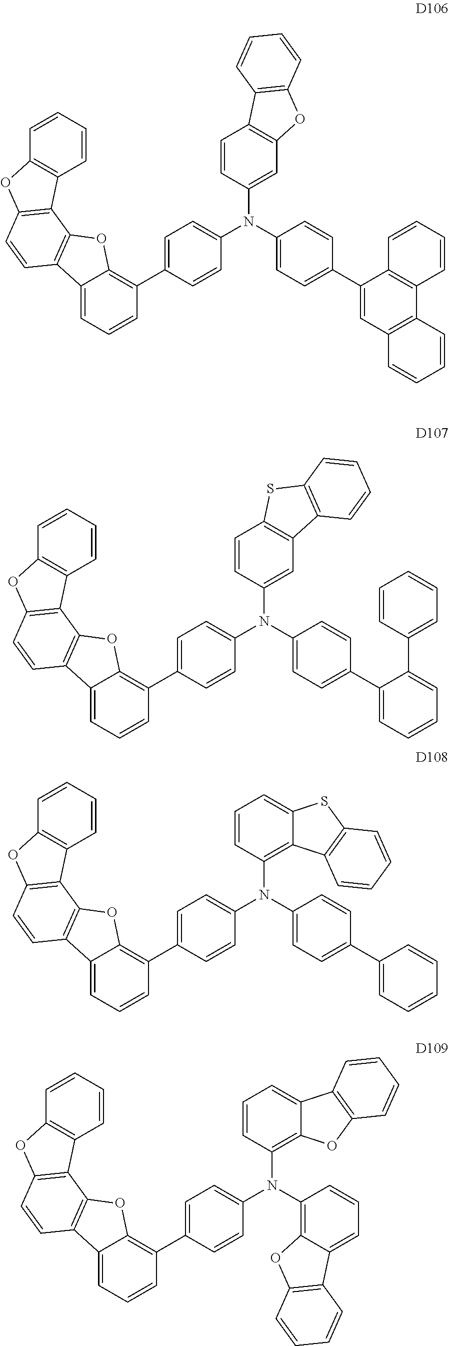 [SMILES: c1ccc2c(c1)cc(-c1ccc(N(c3ccc(-c4cccc5c4oc4c5ccc5oc6ccccc6c54)cc3)c3ccc4c(c3)oc3ccccc34)cc1)c1ccccc12 ; c1ccc(-c2ccccc2-c2ccc(N(c3ccc(-c4cccc5c4oc4c5ccc5oc6ccccc6c54)cc3)c3ccc4sc5ccccc5c4c3)cc2)cc1 ; c1ccc(-c2ccc(N(c3ccc(-c4cccc5c4oc4c5ccc5oc6ccccc6c54)cc3)c3cccc4sc5ccccc5c34)cc2)cc1 ; c1ccc2c(c1)oc1c(N(c3ccc(-c4cccc5c4oc4c5ccc5oc6ccccc6c54)cc3)c3cccc4c3oc3ccccc34)cccc12]

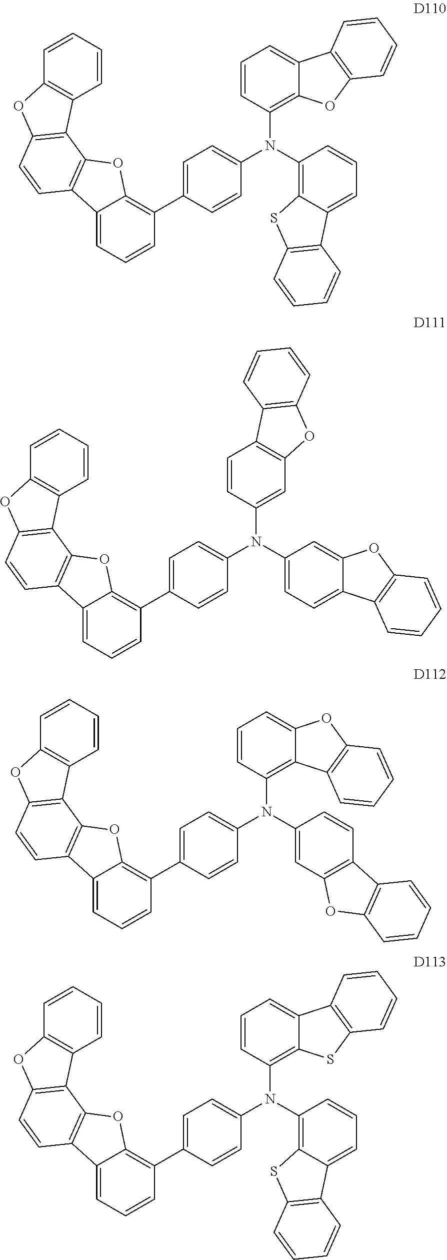

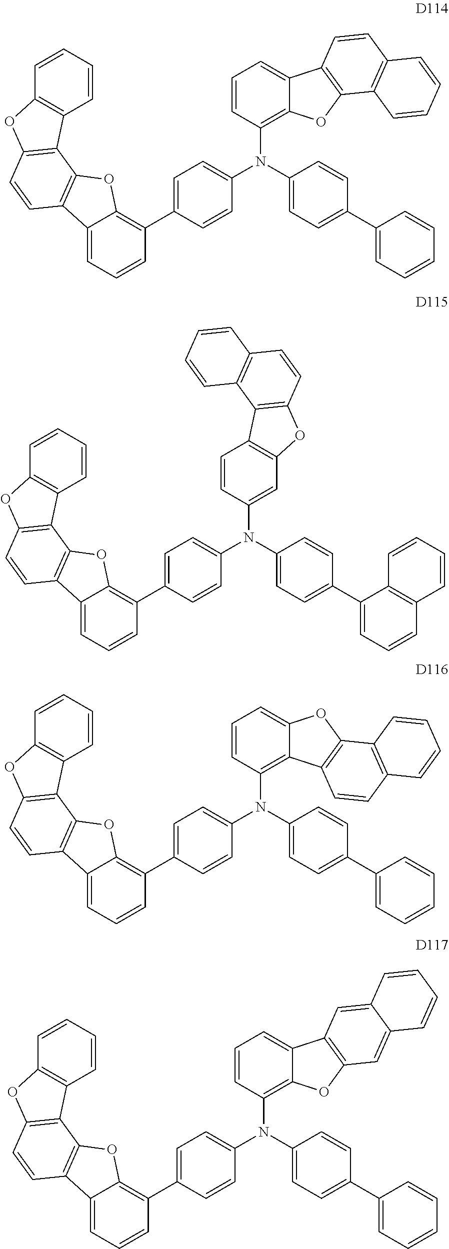 [SMILES: c1ccc(-c2ccc(N(c3ccc(-c4cccc5c4oc4c5ccc5oc6ccccc6c54)cc3)c3cccc4c3oc3c5ccccc5ccc43)cc2)cc1 ; c1ccc2c(-c3ccc(N(c4ccc(-c5cccc6c5oc5c6ccc6oc7ccccc7c65)cc4)c4ccc5c(c4)oc4ccc6ccccc6c45)cc3)cccc2c1 ; c1ccc(-c2ccc(N(c3ccc(-c4cccc5c4oc4c5ccc5oc6ccccc6c54)cc3)c3cccc4oc5c6ccccc6ccc5c34)cc2)cc1 ; c1ccc(-c2ccc(N(c3ccc(-c4cccc5c4oc4c5ccc5oc6ccccc6c54)cc3)c3cccc4c3oc3cc5ccccc5cc34)cc2)cc1]

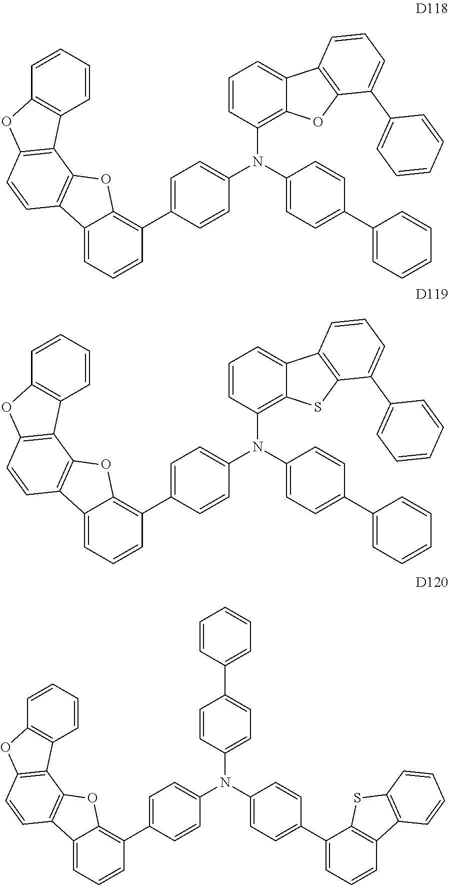 [SMILES: c1ccc(-c2ccc(N(c3ccc(-c4cccc5c4oc4c5ccc5oc6ccccc6c54)cc3)c3cccc4c3oc3c(-c5ccccc5)cccc34)cc2)cc1 ; c1ccc(-c2ccc(N(c3ccc(-c4cccc5c4oc4c5ccc5oc6ccccc6c54)cc3)c3cccc4c3sc3c(-c5ccccc5)cccc34)cc2)cc1 ; c1ccc(-c2ccc(N(c3ccc(-c4cccc5c4oc4c5ccc5oc6ccccc6c54)cc3)c3ccc(-c4cccc5c4sc4ccccc45)cc3)cc2)cc1]

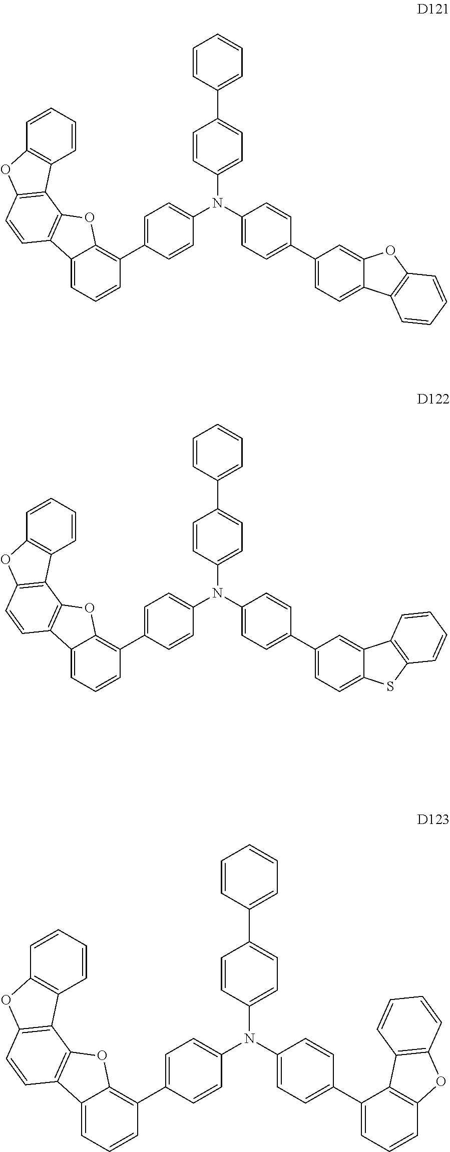 [SMILES: c1ccc(-c2ccc(N(c3ccc(-c4ccc5c(c4)oc4ccccc45)cc3)c3ccc(-c4cccc5c4oc4c5ccc5oc6ccccc6c54)cc3)cc2)cc1 ; c1ccc(-c2ccc(N(c3ccc(-c4ccc5sc6ccccc6c5c4)cc3)c3ccc(-c4cccc5c4oc4c5ccc5oc6ccccc6c54)cc3)cc2)cc1 ; c1ccc(-c2ccc(N(c3ccc(-c4cccc5c4oc4c5ccc5oc6ccccc6c54)cc3)c3ccc(-c4cccc5oc6ccccc6c45)cc3)cc2)cc1]

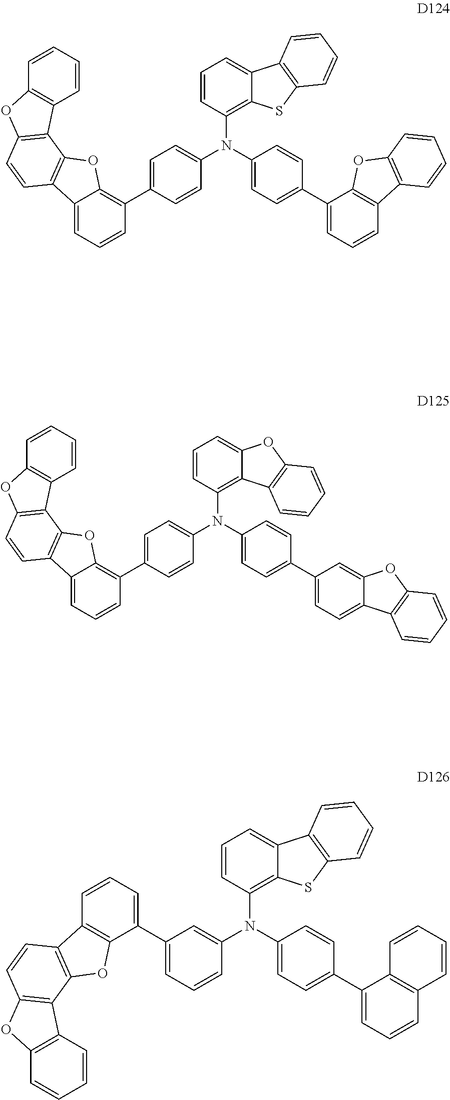 [SMILES: c1ccc2c(c1)oc1c(-c3ccc(N(c4ccc(-c5cccc6c5oc5c6ccc6oc7ccccc7c65)cc4)c4cccc5c4sc4ccccc45)cc3)cccc12 ; c1ccc2c(c1)oc1cc(-c3ccc(N(c4ccc(-c5cccc6c5oc5c6ccc6oc7ccccc7c65)cc4)c4cccc5oc6ccccc6c45)cc3)ccc12 ; c1cc(-c2cccc3c2oc2c3ccc3oc4ccccc4c32)cc(N(c2ccc(-c3cccc4ccccc34)cc2)c2cccc3c2sc2ccccc23)c1]

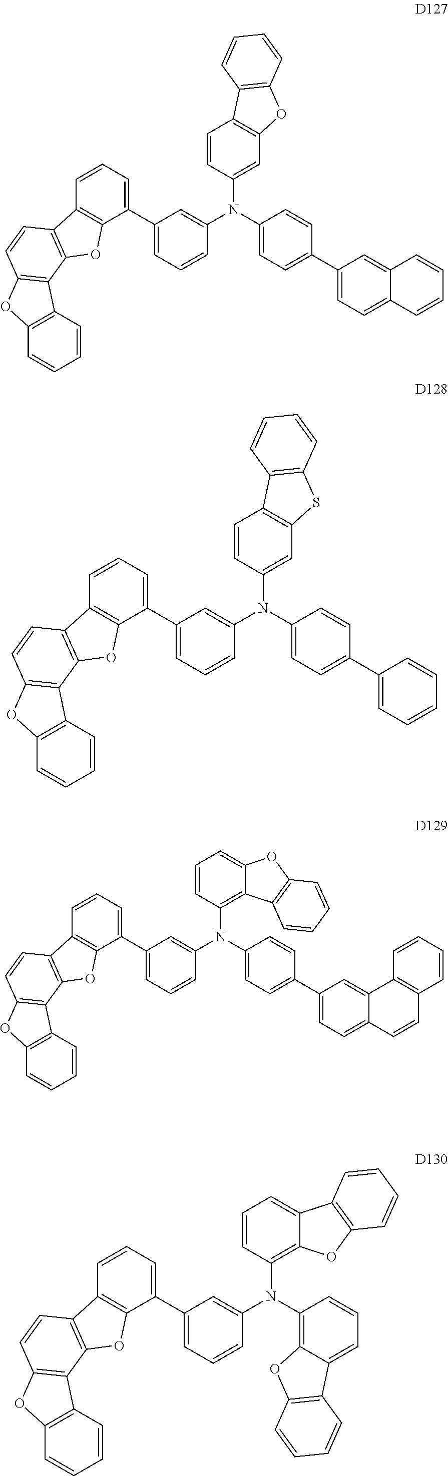 [SMILES: c1cc(-c2cccc3c2oc2c3ccc3oc4ccccc4c32)cc(N(c2ccc(-c3ccc4ccccc4c3)cc2)c2ccc3c(c2)oc2ccccc23)c1 ; c1ccc(-c2ccc(N(c3cccc(-c4cccc5c4oc4c5ccc5oc6ccccc6c54)c3)c3ccc4c(c3)sc3ccccc34)cc2)cc1 ; c1cc(-c2cccc3c2oc2c3ccc3oc4ccccc4c32)cc(N(c2ccc(-c3ccc4ccc5ccccc5c4c3)cc2)c2cccc3oc4ccccc4c23)c1 ; c1cc(-c2cccc3c2oc2c3ccc3oc4ccccc4c32)cc(N(c2cccc3c2oc2ccccc23)c2cccc3c2oc2ccccc23)c1]

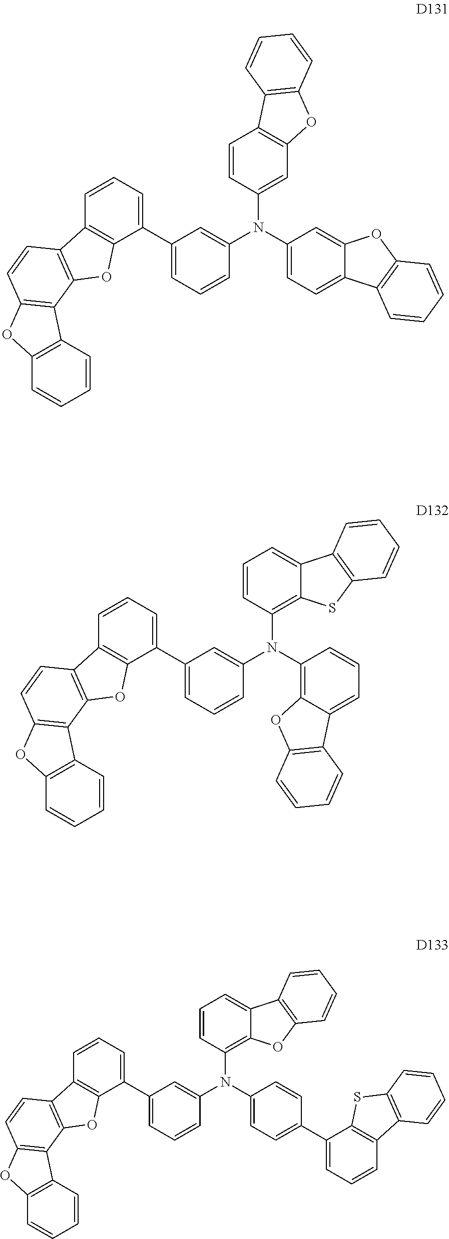 [SMILES: c1cc(-c2cccc3c2oc2c3ccc3oc4ccccc4c32)cc(N(c2ccc3c(c2)oc2ccccc23)c2ccc3c(c2)oc2ccccc23)c1 ; c1cc(-c2cccc3c2oc2c3ccc3oc4ccccc4c32)cc(N(c2cccc3c2oc2ccccc23)c2cccc3c2sc2ccccc23)c1 ; c1cc(-c2cccc3c2oc2c3ccc3oc4ccccc4c32)cc(N(c2ccc(-c3cccc4c3sc3ccccc34)cc2)c2cccc3c2oc2ccccc23)c1]

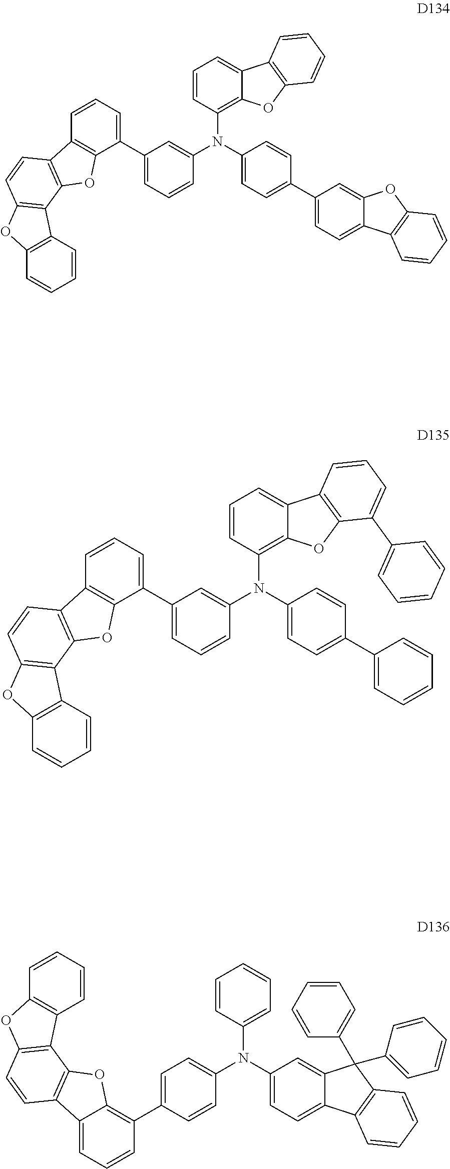 [SMILES: c1cc(-c2cccc3c2oc2c3ccc3oc4ccccc4c32)cc(N(c2ccc(-c3ccc4c(c3)oc3ccccc34)cc2)c2cccc3c2oc2ccccc23)c1 ; c1ccc(-c2ccc(N(c3cccc(-c4cccc5c4oc4c5ccc5oc6ccccc6c54)c3)c3cccc4c3oc3c(-c5ccccc5)cccc34)cc2)cc1 ; c1ccc(N(c2ccc(-c3cccc4c3oc3c4ccc4oc5ccccc5c43)cc2)c2ccc3c(c2)C(c2ccccc2)(c2ccccc2)c2ccccc2-3)cc1]

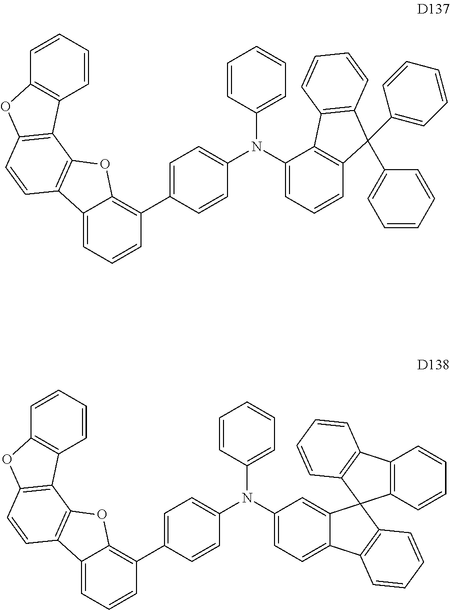 [SMILES: c1ccc(N(c2ccc(-c3cccc4c3oc3c4ccc4oc5ccccc5c43)cc2)c2cccc3c2-c2ccccc2C3(c2ccccc2)c2ccccc2)cc1 ; c1ccc(N(c2ccc(-c3cccc4c3oc3c4ccc4oc5ccccc5c43)cc2)c2ccc3c(c2)C2(c4ccccc4-c4ccccc42)c2ccccc2-3)cc1]

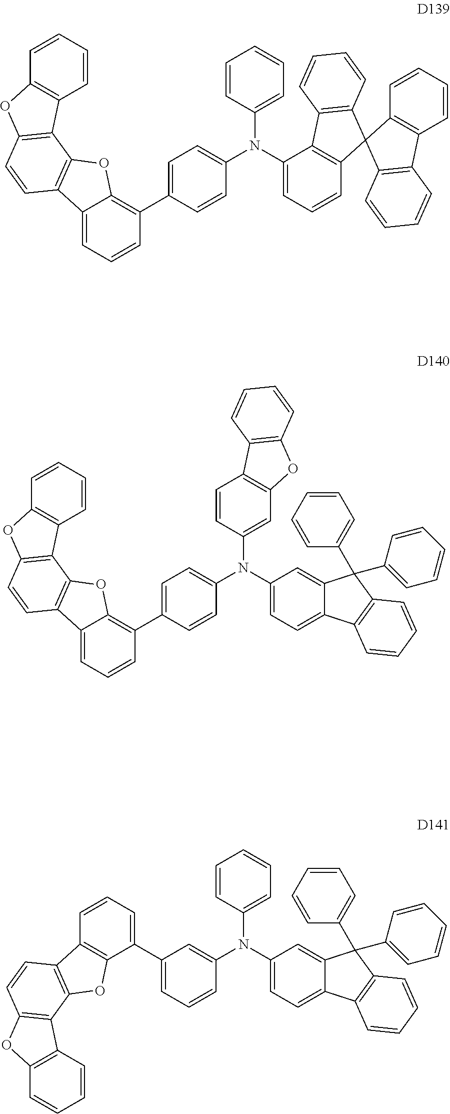 [SMILES: c1ccc(N(c2ccc(-c3cccc4c3oc3c4ccc4oc5ccccc5c43)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6c5oc5c6ccc6oc7ccccc7c65)cc4)c4ccc5c(c4)oc4ccccc45)cc32)cc1 ; c1ccc(N(c2cccc(-c3cccc4c3oc3c4ccc4oc5ccccc5c43)c2)c2ccc3c(c2)C(c2ccccc2)(c2ccccc2)c2ccccc2-3)cc1]

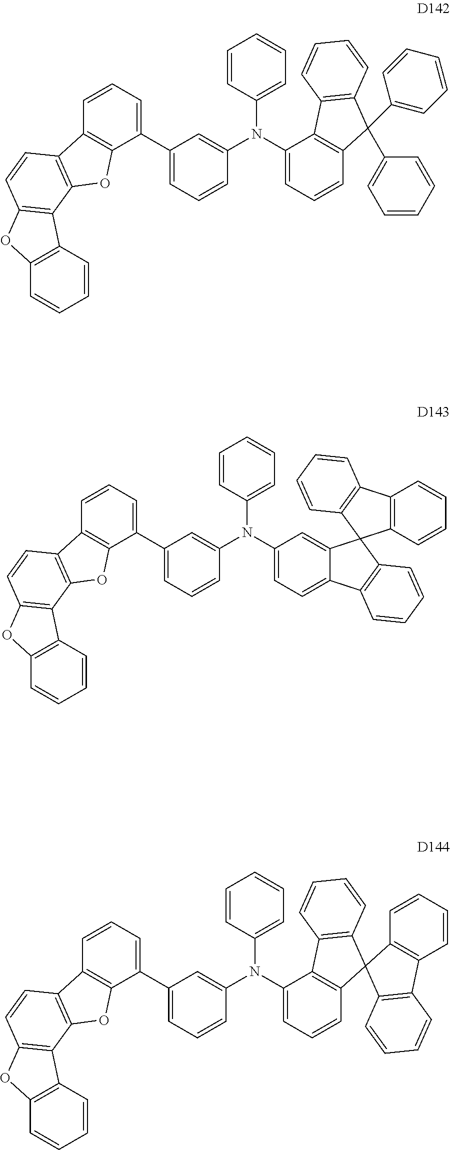 [SMILES: c1ccc(N(c2cccc(-c3cccc4c3oc3c4ccc4oc5ccccc5c43)c2)c2cccc3c2-c2ccccc2C3(c2ccccc2)c2ccccc2)cc1 ; c1ccc(N(c2cccc(-c3cccc4c3oc3c4ccc4oc5ccccc5c43)c2)c2ccc3c(c2)C2(c4ccccc4-c4ccccc42)c2ccccc2-3)cc1 ; c1ccc(N(c2cccc(-c3cccc4c3oc3c4ccc4oc5ccccc5c43)c2)c2cccc3c2-c2ccccc2C32c3ccccc3-c3ccccc32)cc1]

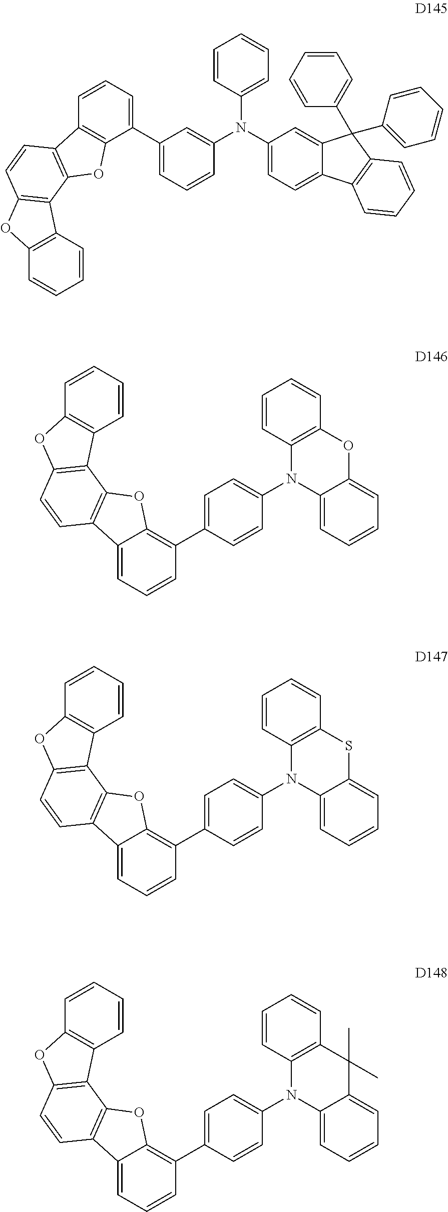 [SMILES: c1ccc(N(c2cccc(-c3cccc4c3oc3c4ccc4oc5ccccc5c43)c2)c2ccc3c(c2)C(c2ccccc2)(c2ccccc2)c2ccccc2-3)cc1 ; c1ccc2c(c1)Oc1ccccc1N2c1ccc(-c2cccc3c2oc2c3ccc3oc4ccccc4c32)cc1 ; c1ccc2c(c1)Sc1ccccc1N2c1ccc(-c2cccc3c2oc2c3ccc3oc4ccccc4c32)cc1 ; CC1(C)c2ccccc2N(c2ccc(-c3cccc4c3oc3c4ccc4oc5ccccc5c43)cc2)c2ccccc21]

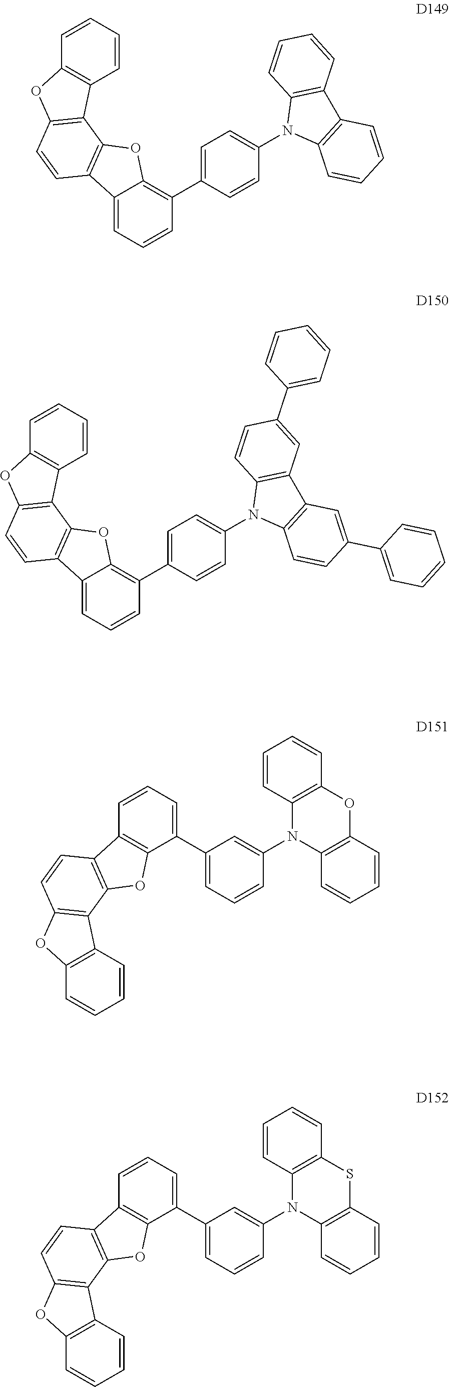 [SMILES: c1ccc2c(c1)oc1ccc3c4cccc(-c5ccc(-n6c7ccccc7c7ccccc76)cc5)c4oc3c12 ; c1ccc(-c2ccc3c(c2)c2cc(-c4ccccc4)ccc2n3-c2ccc(-c3cccc4c3oc3c4ccc4oc5ccccc5c43)cc2)cc1 ; c1cc(-c2cccc3c2oc2c3ccc3oc4ccccc4c32)cc(N2c3ccccc3Oc3ccccc32)c1 ; c1cc(-c2cccc3c2oc2c3ccc3oc4ccccc4c32)cc(N2c3ccccc3Sc3ccccc32)c1]

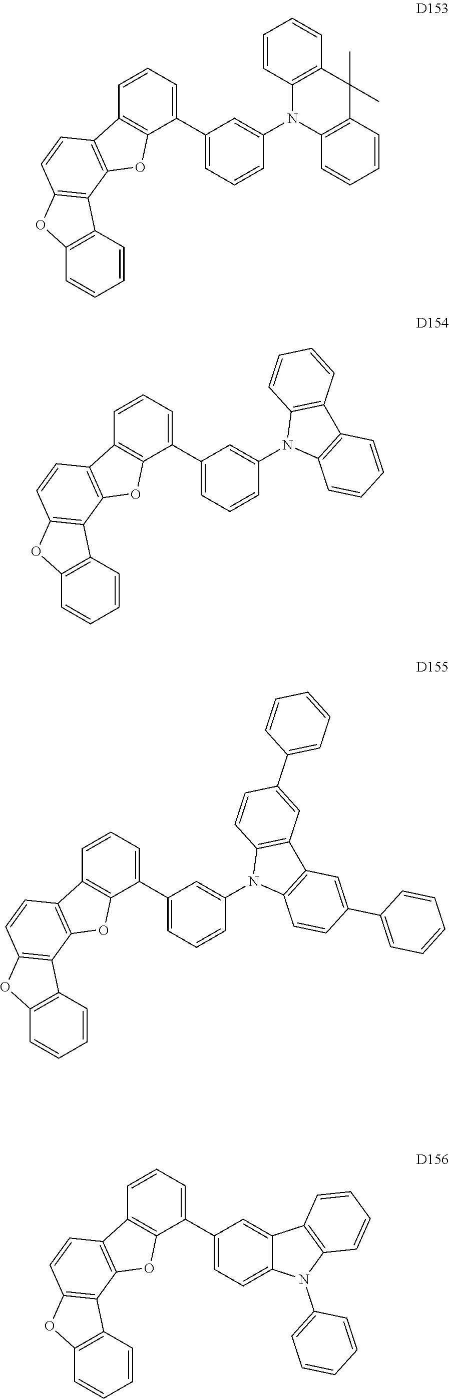 [SMILES: CC1(C)c2ccccc2N(c2cccc(-c3cccc4c3oc3c4ccc4oc5ccccc5c43)c2)c2ccccc21 ; c1cc(-c2cccc3c2oc2c3ccc3oc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3oc3c4ccc4oc5ccccc5c43)c2)cc1 ; c1ccc(-n2c3ccccc3c3cc(-c4cccc5c4oc4c5ccc5oc6ccccc6c54)ccc32)cc1]

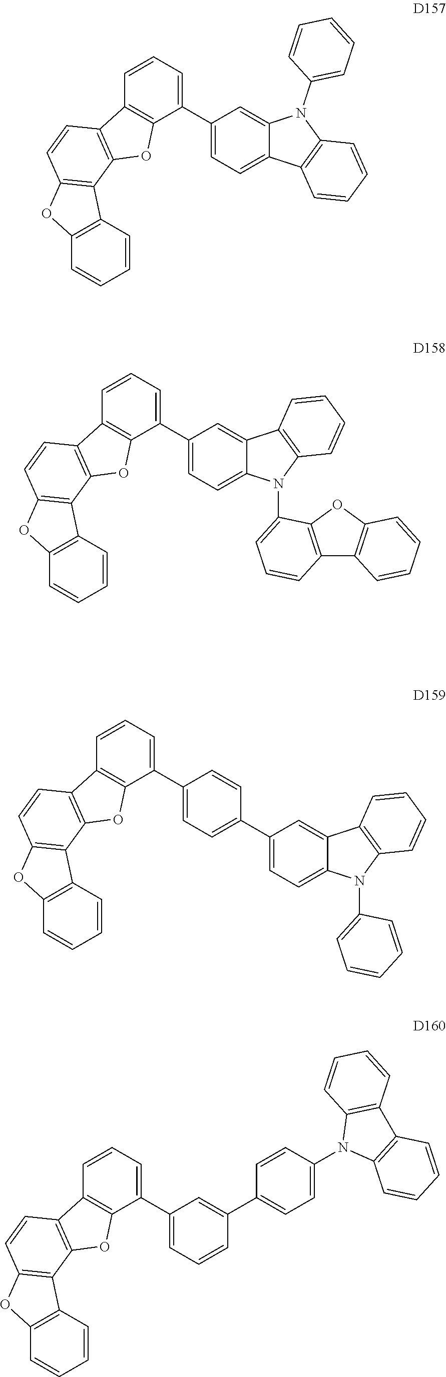 [SMILES: c1ccc(-n2c3ccccc3c3ccc(-c4cccc5c4oc4c5ccc5oc6ccccc6c54)cc32)cc1 ; c1ccc2c(c1)oc1c(-n3c4ccccc4c4cc(-c5cccc6c5oc5c6ccc6oc7ccccc7c65)ccc43)cccc12 ; c1ccc(-n2c3ccccc3c3cc(-c4ccc(-c5cccc6c5oc5c6ccc6oc7ccccc7c65)cc4)ccc32)cc1 ; c1cc(-c2ccc(-n3c4ccccc4c4ccccc43)cc2)cc(-c2cccc3c2oc2c3ccc3oc4ccccc4c32)c1]

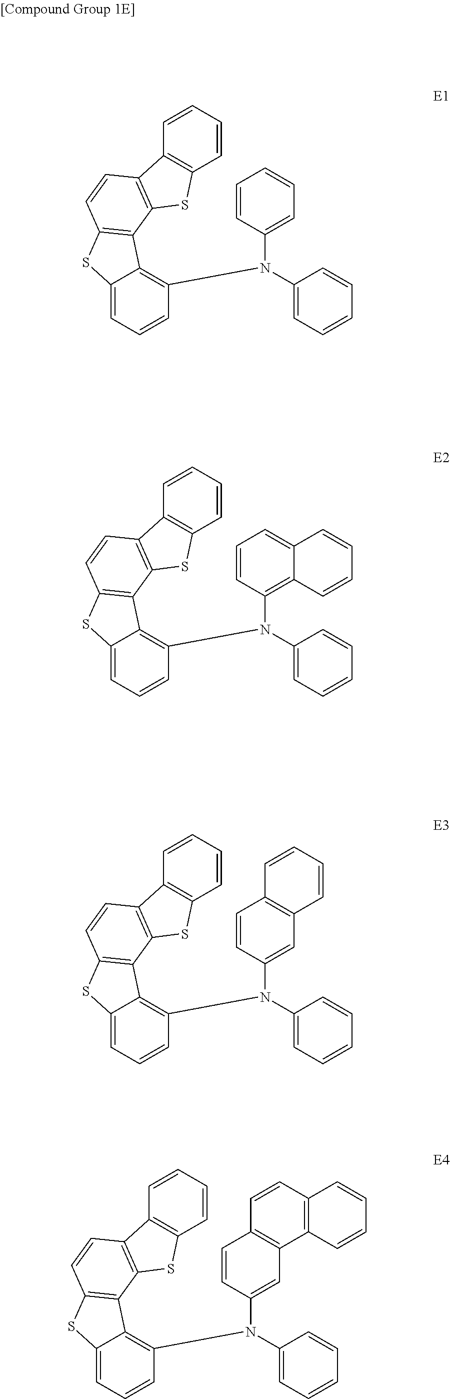 [SMILES: c1ccc(N(c2ccccc2)c2cccc3sc4ccc5c6ccccc6sc5c4c23)cc1 ; c1ccc(N(c2cccc3ccccc23)c2cccc3sc4ccc5c6ccccc6sc5c4c23)cc1 ; c1ccc(N(c2ccc3ccccc3c2)c2cccc3sc4ccc5c6ccccc6sc5c4c23)cc1 ; c1ccc(N(c2ccc3ccc4ccccc4c3c2)c2cccc3sc4ccc5c6ccccc6sc5c4c23)cc1]

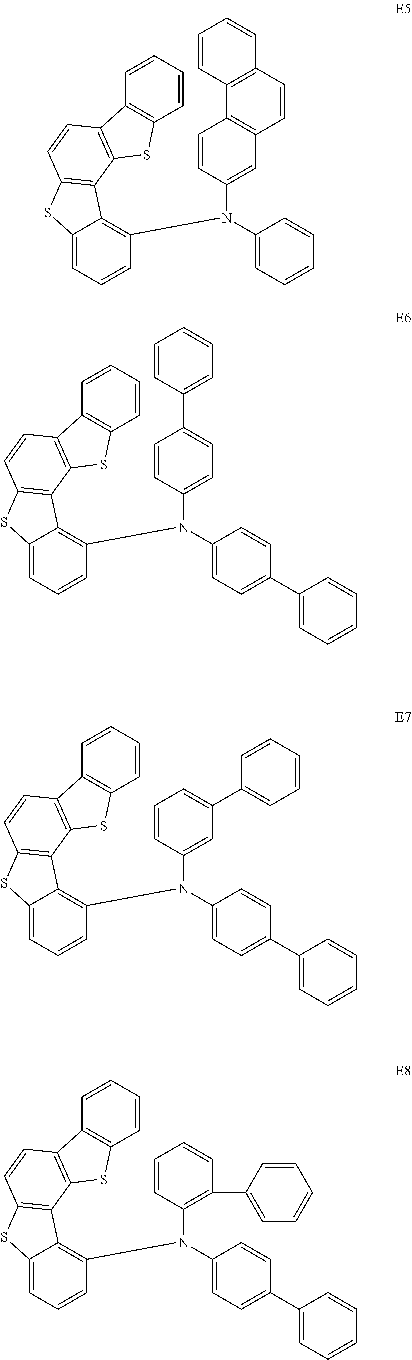 [SMILES: c1ccc(N(c2ccc3c(ccc4ccccc43)c2)c2cccc3sc4ccc5c6ccccc6sc5c4c23)cc1 ; c1ccc(-c2ccc(N(c3ccc(-c4ccccc4)cc3)c3cccc4sc5ccc6c7ccccc7sc6c5c34)cc2)cc1 ; c1ccc(-c2ccc(N(c3cccc(-c4ccccc4)c3)c3cccc4sc5ccc6c7ccccc7sc6c5c34)cc2)cc1 ; c1ccc(-c2ccc(N(c3ccccc3-c3ccccc3)c3cccc4sc5ccc6c7ccccc7sc6c5c34)cc2)cc1]

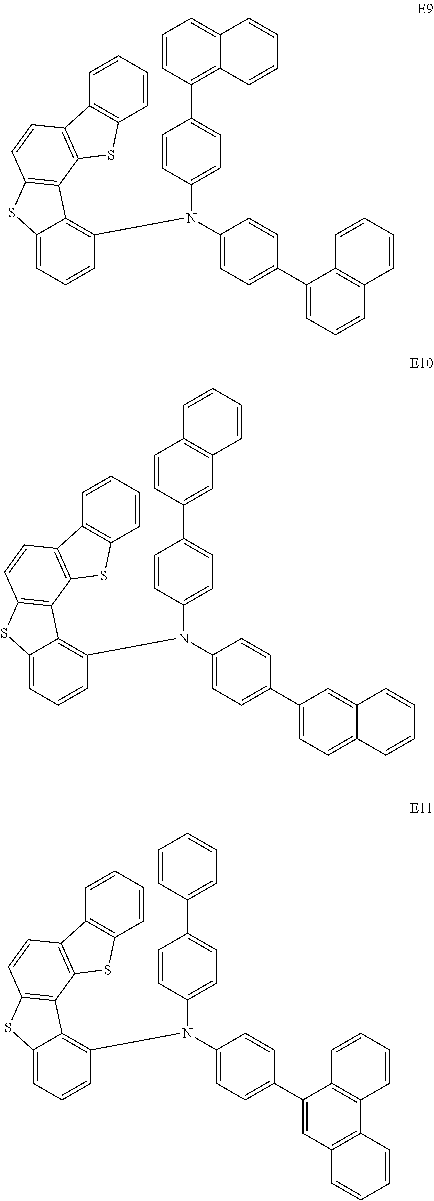 [SMILES: c1ccc2c(-c3ccc(N(c4ccc(-c5cccc6ccccc56)cc4)c4cccc5sc6ccc7c8ccccc8sc7c6c45)cc3)cccc2c1 ; c1ccc2cc(-c3ccc(N(c4ccc(-c5ccc6ccccc6c5)cc4)c4cccc5sc6ccc7c8ccccc8sc7c6c45)cc3)ccc2c1 ; c1ccc(-c2ccc(N(c3ccc(-c4cc5ccccc5c5ccccc45)cc3)c3cccc4sc5ccc6c7ccccc7sc6c5c34)cc2)cc1]

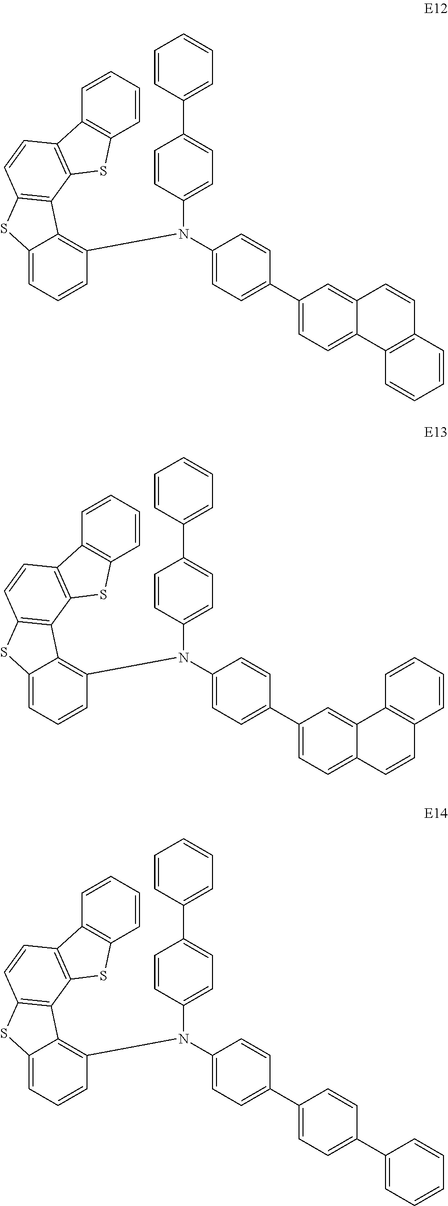 [SMILES: c1ccc(-c2ccc(N(c3ccc(-c4ccc5c(ccc6ccccc65)c4)cc3)c3cccc4sc5ccc6c7ccccc7sc6c5c34)cc2)cc1 ; c1ccc(-c2ccc(N(c3ccc(-c4ccc5ccc6ccccc6c5c4)cc3)c3cccc4sc5ccc6c7ccccc7sc6c5c34)cc2)cc1 ; c1ccc(-c2ccc(-c3ccc(N(c4ccc(-c5ccccc5)cc4)c4cccc5sc6ccc7c8ccccc8sc7c6c45)cc3)cc2)cc1]

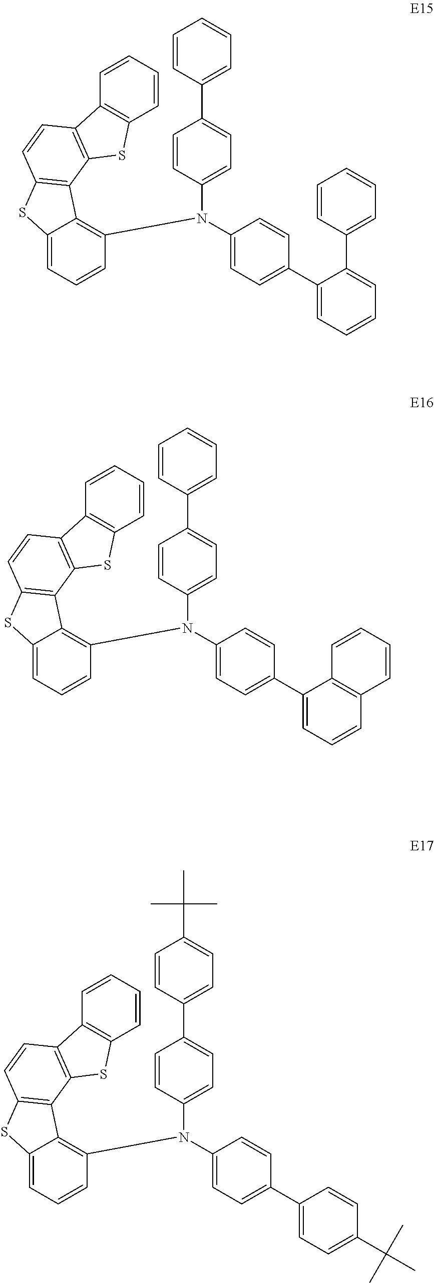 [SMILES: c1ccc(-c2ccc(N(c3ccc(-c4ccccc4-c4ccccc4)cc3)c3cccc4sc5ccc6c7ccccc7sc6c5c34)cc2)cc1 ; c1ccc(-c2ccc(N(c3ccc(-c4cccc5ccccc45)cc3)c3cccc4sc5ccc6c7ccccc7sc6c5c34)cc2)cc1 ; CC(C)(C)c1ccc(-c2ccc(N(c3ccc(-c4ccc(C(C)(C)C)cc4)cc3)c3cccc4sc5ccc6c7ccccc7sc6c5c34)cc2)cc1]

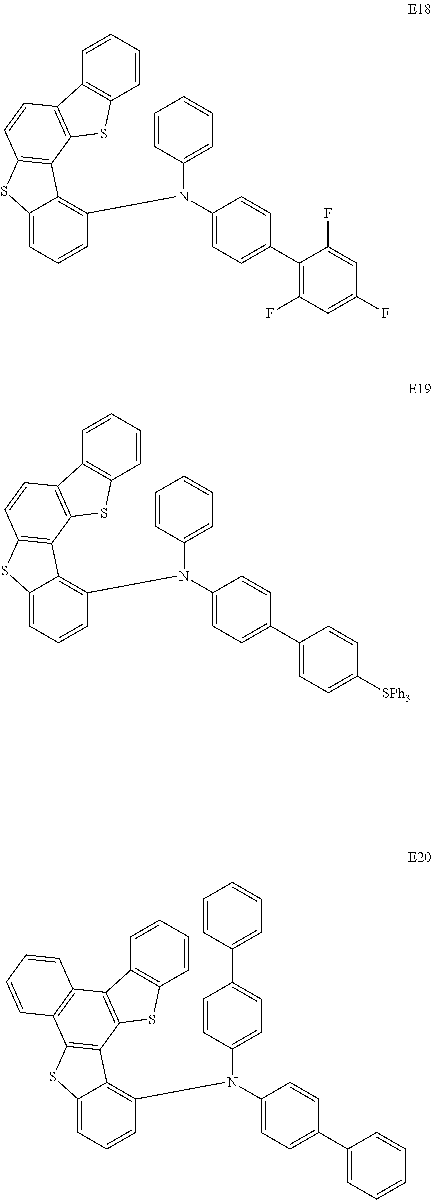

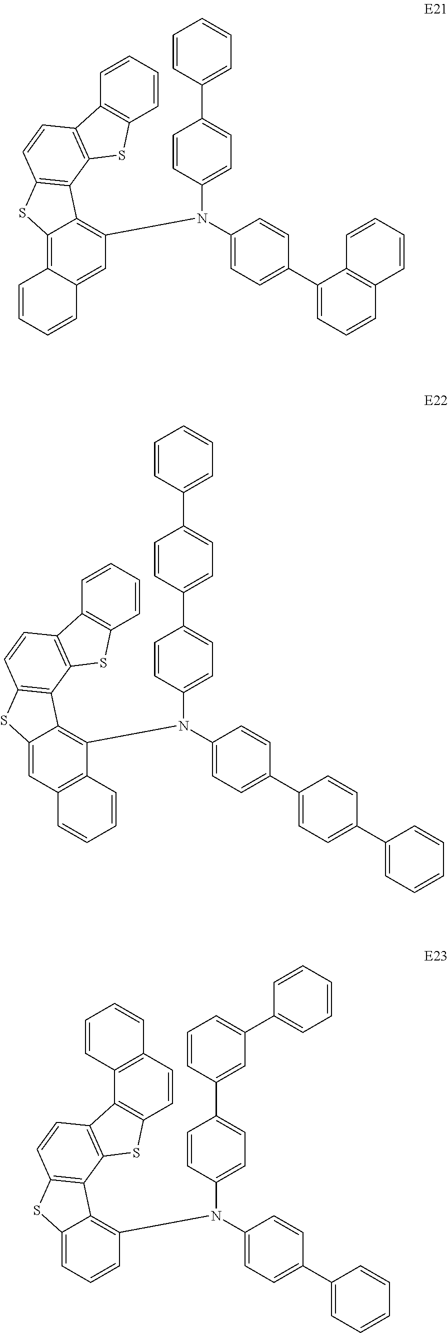 [SMILES: c1ccc(-c2ccc(N(c3ccc(-c4cccc5ccccc45)cc3)c3cc4ccccc4c4sc5ccc6c7ccccc7sc6c5c34)cc2)cc1 ; c1ccc(-c2ccc(-c3ccc(N(c4ccc(-c5ccc(-c6ccccc6)cc5)cc4)c4c5ccccc5cc5sc6ccc7c8ccccc8sc7c6c45)cc3)cc2)cc1 ; c1ccc(-c2ccc(N(c3ccc(-c4cccc(-c5ccccc5)c4)cc3)c3cccc4sc5ccc6c(sc7ccc8ccccc8c76)c5c34)cc2)cc1]

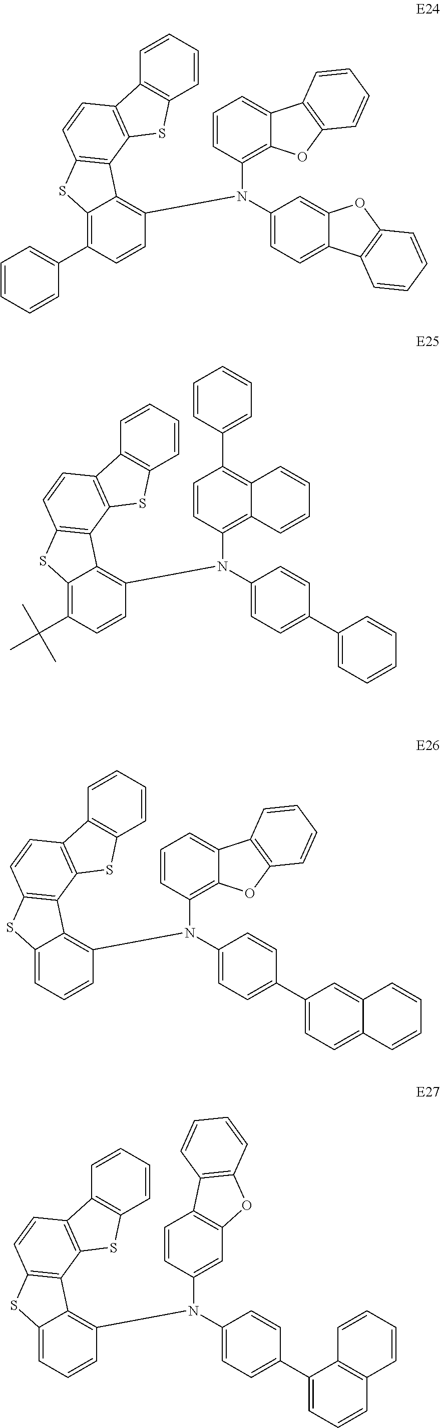 [SMILES: c1ccc(-c2ccc(N(c3ccc4c(c3)oc3ccccc34)c3cccc4c3oc3ccccc34)c3c2sc2ccc4c5ccccc5sc4c23)cc1 ; CC(C)(C)c1ccc(N(c2ccc(-c3ccccc3)cc2)c2ccc(-c3ccccc3)c3ccccc23)c2c1sc1ccc3c4ccccc4sc3c12 ; c1ccc2cc(-c3ccc(N(c4cccc5c4oc4ccccc45)c4cccc5sc6ccc7c8ccccc8sc7c6c45)cc3)ccc2c1 ; c1ccc2c(-c3ccc(N(c4ccc5c(c4)oc4ccccc45)c4cccc5sc6ccc7c8ccccc8sc7c6c45)cc3)cccc2c1]

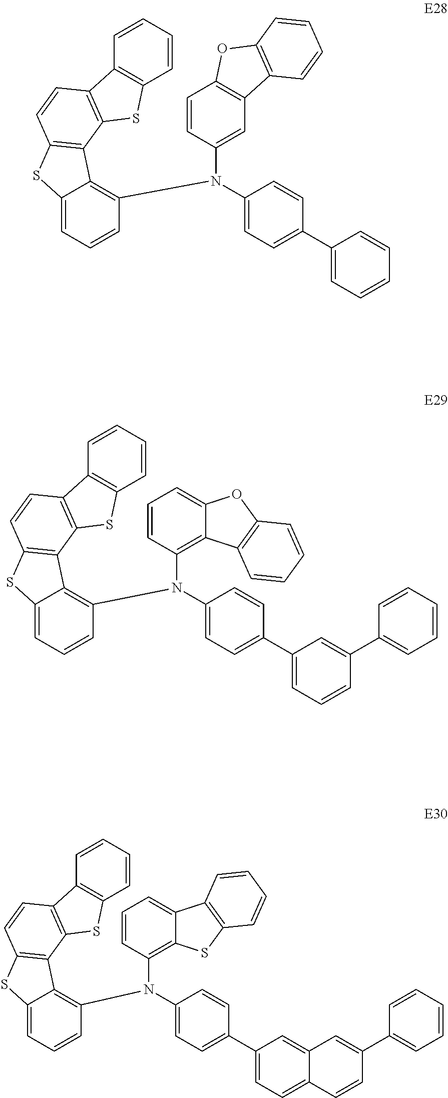 [SMILES: c1ccc(-c2ccc(N(c3ccc4oc5ccccc5c4c3)c3cccc4sc5ccc6c7ccccc7sc6c5c34)cc2)cc1 ; c1ccc(-c2cccc(-c3ccc(N(c4cccc5oc6ccccc6c45)c4cccc5sc6ccc7c8ccccc8sc7c6c45)cc3)c2)cc1 ; c1ccc(-c2ccc3ccc(-c4ccc(N(c5cccc6c5sc5ccccc56)c5cccc6sc7ccc8c9ccccc9sc8c7c56)cc4)cc3c2)cc1]

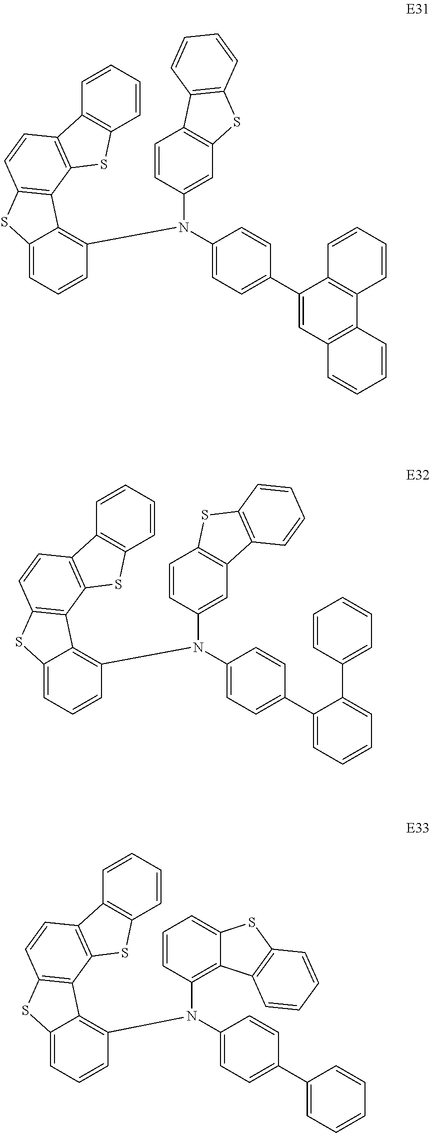 [SMILES: c1ccc2c(c1)cc(-c1ccc(N(c3ccc4c(c3)sc3ccccc34)c3cccc4sc5ccc6c7ccccc7sc6c5c34)cc1)c1ccccc12 ; c1ccc(-c2ccccc2-c2ccc(N(c3ccc4sc5ccccc5c4c3)c3cccc4sc5ccc6c7ccccc7sc6c5c34)cc2)cc1 ; c1ccc(-c2ccc(N(c3cccc4sc5ccccc5c34)c3cccc4sc5ccc6c7ccccc7sc6c5c34)cc2)cc1]

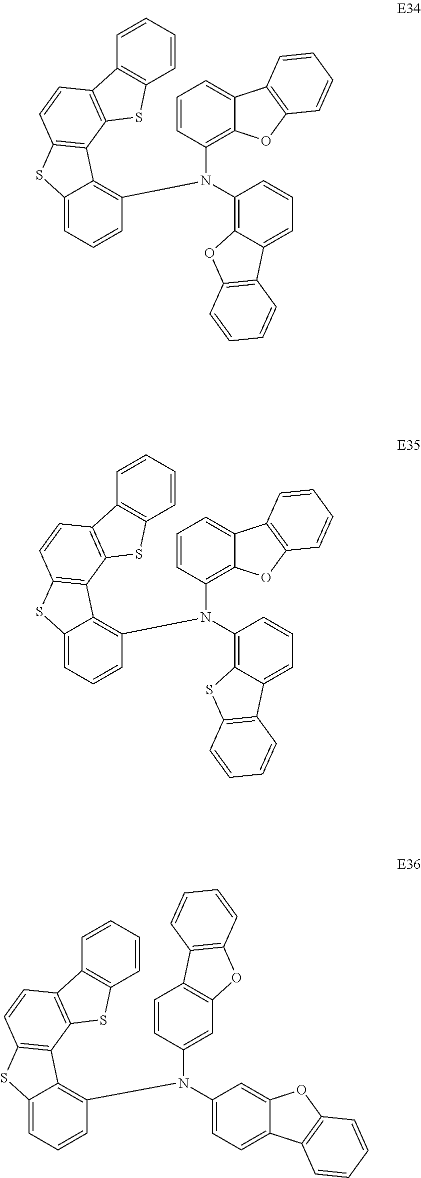 [SMILES: c1ccc2c(c1)oc1c(N(c3cccc4c3oc3ccccc34)c3cccc4sc5ccc6c7ccccc7sc6c5c34)cccc12 ; c1ccc2c(c1)oc1c(N(c3cccc4c3sc3ccccc34)c3cccc4sc5ccc6c7ccccc7sc6c5c34)cccc12 ; c1ccc2c(c1)oc1cc(N(c3ccc4c(c3)oc3ccccc34)c3cccc4sc5ccc6c7ccccc7sc6c5c34)ccc12]

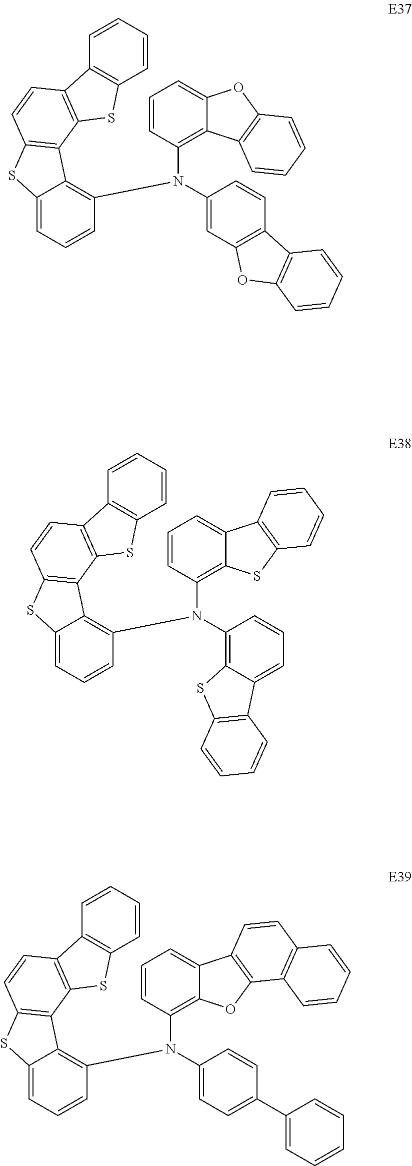 [SMILES: c1ccc2c(c1)oc1cc(N(c3cccc4oc5ccccc5c34)c3cccc4sc5ccc6c7ccccc7sc6c5c34)ccc12 ; c1ccc2c(c1)sc1c(N(c3cccc4c3sc3ccccc34)c3cccc4sc5ccc6c7ccccc7sc6c5c34)cccc12 ; c1ccc(-c2ccc(N(c3cccc4c3oc3c5ccccc5ccc43)c3cccc4sc5ccc6c7ccccc7sc6c5c34)cc2)cc1]

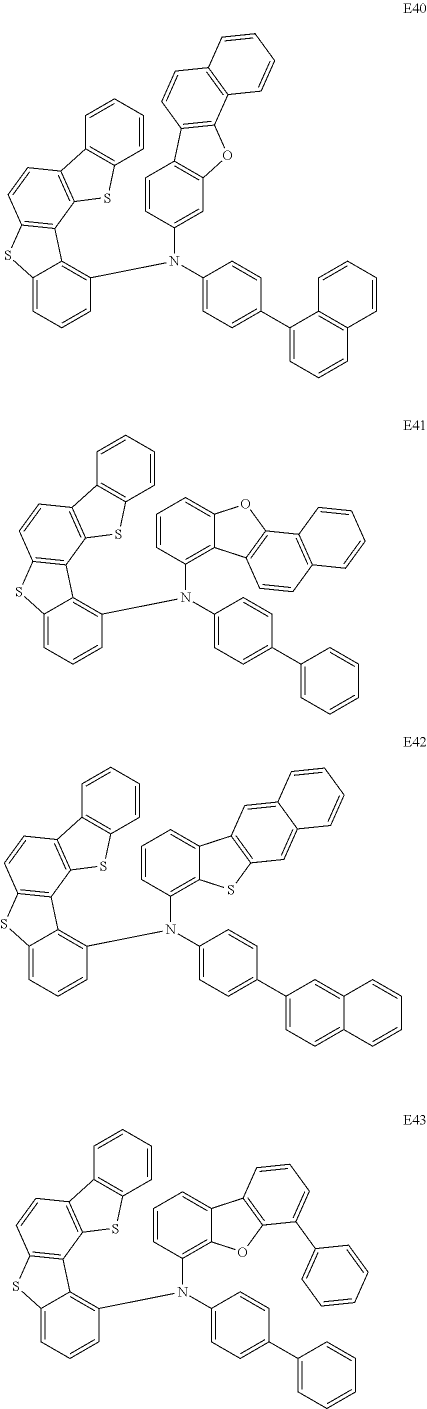 [SMILES: c1ccc2c(-c3ccc(N(c4ccc5c(c4)oc4c6ccccc6ccc54)c4cccc5sc6ccc7c8ccccc8sc7c6c45)cc3)cccc2c1 ; c1ccc(-c2ccc(N(c3cccc4oc5c6ccccc6ccc5c34)c3cccc4sc5ccc6c7ccccc7sc6c5c34)cc2)cc1 ; c1ccc2cc(-c3ccc(N(c4cccc5c4sc4cc6ccccc6cc45)c4cccc5sc6ccc7c8ccccc8sc7c6c45)cc3)ccc2c1 ; c1ccc(-c2ccc(N(c3cccc4c3oc3c(-c5ccccc5)cccc34)c3cccc4sc5ccc6c7ccccc7sc6c5c34)cc2)cc1]

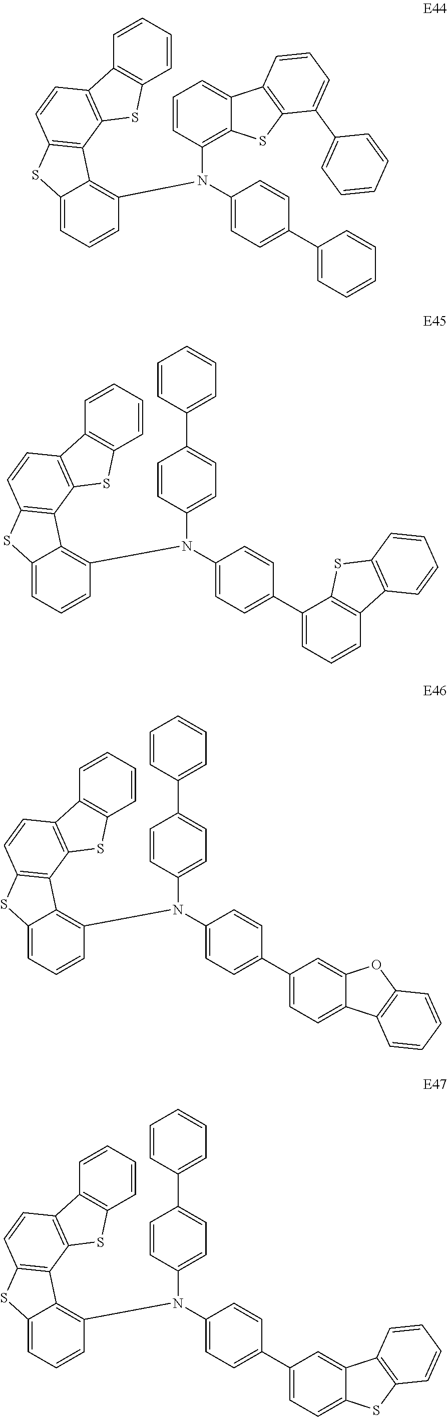 [SMILES: c1ccc(-c2ccc(N(c3cccc4c3sc3c(-c5ccccc5)cccc34)c3cccc4sc5ccc6c7ccccc7sc6c5c34)cc2)cc1 ; c1ccc(-c2ccc(N(c3ccc(-c4cccc5c4sc4ccccc45)cc3)c3cccc4sc5ccc6c7ccccc7sc6c5c34)cc2)cc1 ; c1ccc(-c2ccc(N(c3ccc(-c4ccc5c(c4)oc4ccccc45)cc3)c3cccc4sc5ccc6c7ccccc7sc6c5c34)cc2)cc1 ; c1ccc(-c2ccc(N(c3ccc(-c4ccc5sc6ccccc6c5c4)cc3)c3cccc4sc5ccc6c7ccccc7sc6c5c34)cc2)cc1]

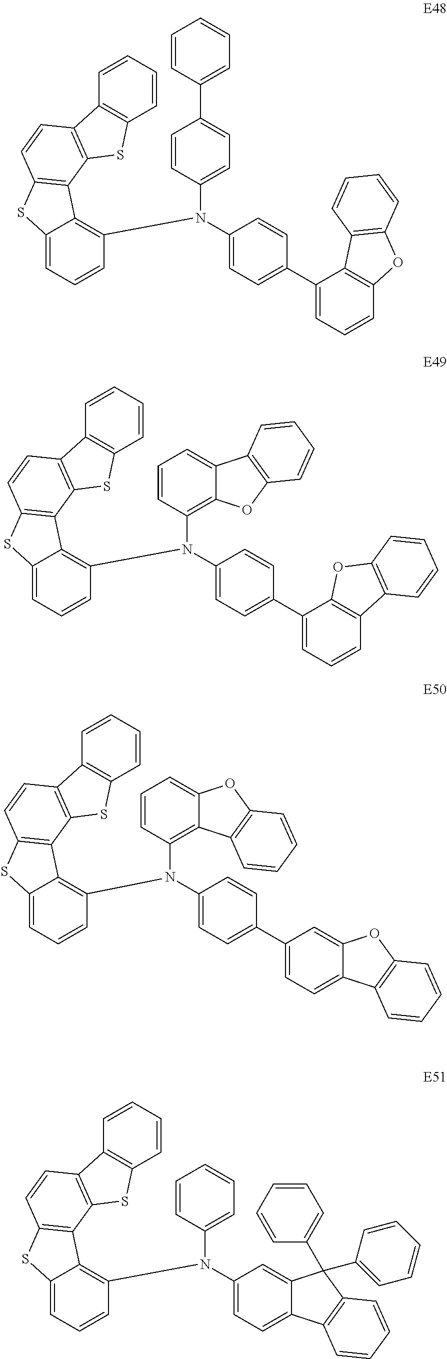 [SMILES: c1ccc(-c2ccc(N(c3ccc(-c4cccc5oc6ccccc6c45)cc3)c3cccc4sc5ccc6c7ccccc7sc6c5c34)cc2)cc1 ; c1ccc2c(c1)oc1c(-c3ccc(N(c4cccc5c4oc4ccccc45)c4cccc5sc6ccc7c8ccccc8sc7c6c45)cc3)cccc12 ; c1ccc2c(c1)oc1cc(-c3ccc(N(c4cccc5oc6ccccc6c45)c4cccc5sc6ccc7c8ccccc8sc7c6c45)cc3)ccc12 ; c1ccc(N(c2ccc3c(c2)C(c2ccccc2)(c2ccccc2)c2ccccc2-3)c2cccc3sc4ccc5c6ccccc6sc5c4c23)cc1]

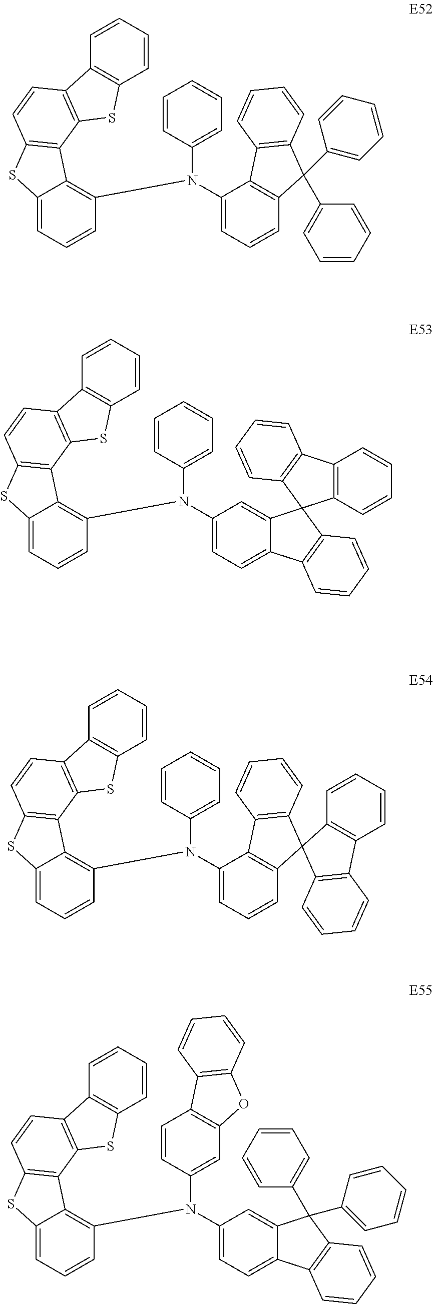 [SMILES: c1ccc(N(c2cccc3c2-c2ccccc2C3(c2ccccc2)c2ccccc2)c2cccc3sc4ccc5c6ccccc6sc5c4c23)cc1 ; c1ccc(N(c2ccc3c(c2)C2(c4ccccc4-c4ccccc42)c2ccccc2-3)c2cccc3sc4ccc5c6ccccc6sc5c4c23)cc1 ; c1ccc(N(c2cccc3c2-c2ccccc2C32c3ccccc3-c3ccccc32)c2cccc3sc4ccc5c6ccccc6sc5c4c23)cc1 ; c1ccc(C2(c3ccccc3)c3ccccc3-c3ccc(N(c4ccc5c(c4)oc4ccccc45)c4cccc5sc6ccc7c8ccccc8sc7c6c45)cc32)cc1]

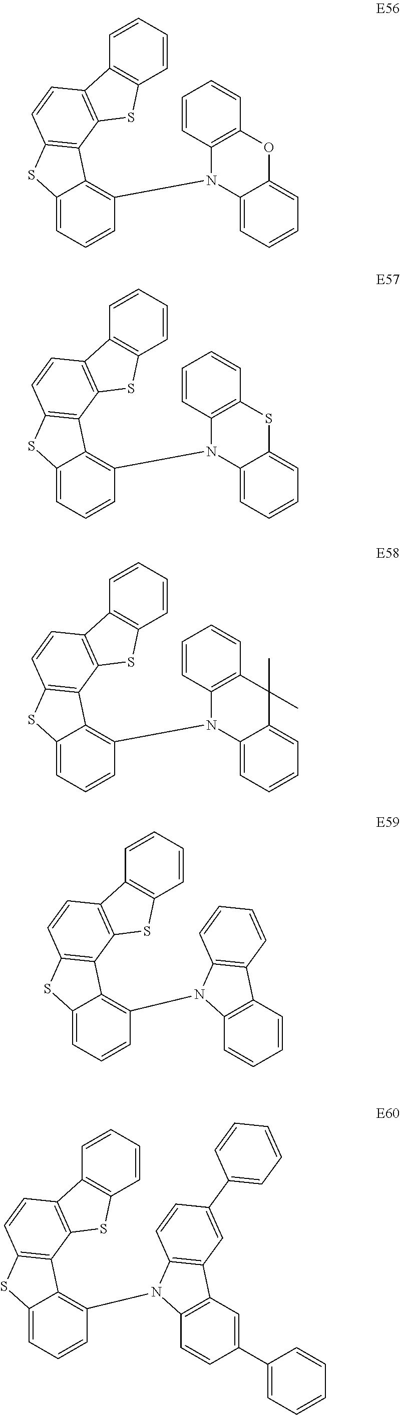 [SMILES: c1ccc2c(c1)Oc1ccccc1N2c1cccc2sc3ccc4c5ccccc5sc4c3c12 ; c1ccc2c(c1)Sc1ccccc1N2c1cccc2sc3ccc4c5ccccc5sc4c3c12 ; CC1(C)c2ccccc2N(c2cccc3sc4ccc5c6ccccc6sc5c4c23)c2ccccc21 ; c1ccc2c(c1)sc1c2ccc2sc3cccc(-n4c5ccccc5c5ccccc54)c3c21 ; c1ccc(-c2ccc3c(c2)c2cc(-c4ccccc4)ccc2n3-c2cccc3sc4ccc5c6ccccc6sc5c4c23)cc1]

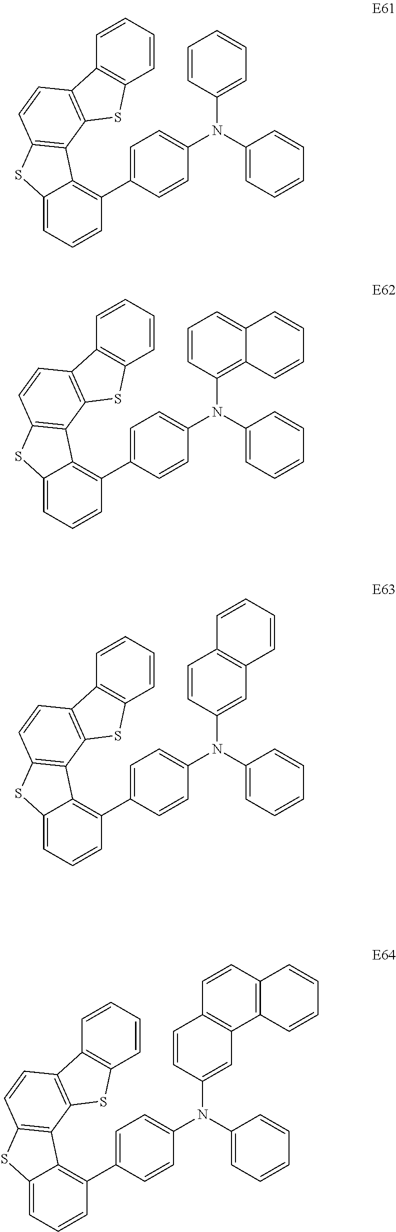 [SMILES: c1ccc(N(c2ccccc2)c2ccc(-c3cccc4sc5ccc6c7ccccc7sc6c5c34)cc2)cc1 ; c1ccc(N(c2ccc(-c3cccc4sc5ccc6c7ccccc7sc6c5c34)cc2)c2cccc3ccccc23)cc1 ; c1ccc(N(c2ccc(-c3cccc4sc5ccc6c7ccccc7sc6c5c34)cc2)c2ccc3ccccc3c2)cc1 ; c1ccc(N(c2ccc(-c3cccc4sc5ccc6c7ccccc7sc6c5c34)cc2)c2ccc3ccc4ccccc4c3c2)cc1]

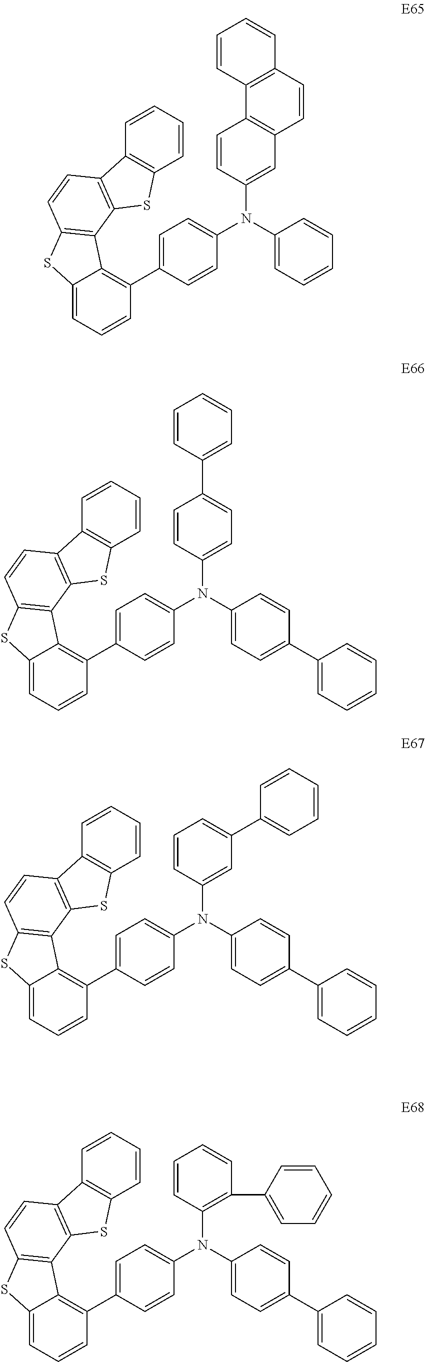 [SMILES: c1ccc(N(c2ccc(-c3cccc4sc5ccc6c7ccccc7sc6c5c34)cc2)c2ccc3c(ccc4ccccc43)c2)cc1 ; c1ccc(-c2ccc(N(c3ccc(-c4ccccc4)cc3)c3ccc(-c4cccc5sc6ccc7c8ccccc8sc7c6c45)cc3)cc2)cc1 ; c1ccc(-c2ccc(N(c3ccc(-c4cccc5sc6ccc7c8ccccc8sc7c6c45)cc3)c3cccc(-c4ccccc4)c3)cc2)cc1 ; c1ccc(-c2ccc(N(c3ccc(-c4cccc5sc6ccc7c8ccccc8sc7c6c45)cc3)c3ccccc3-c3ccccc3)cc2)cc1]

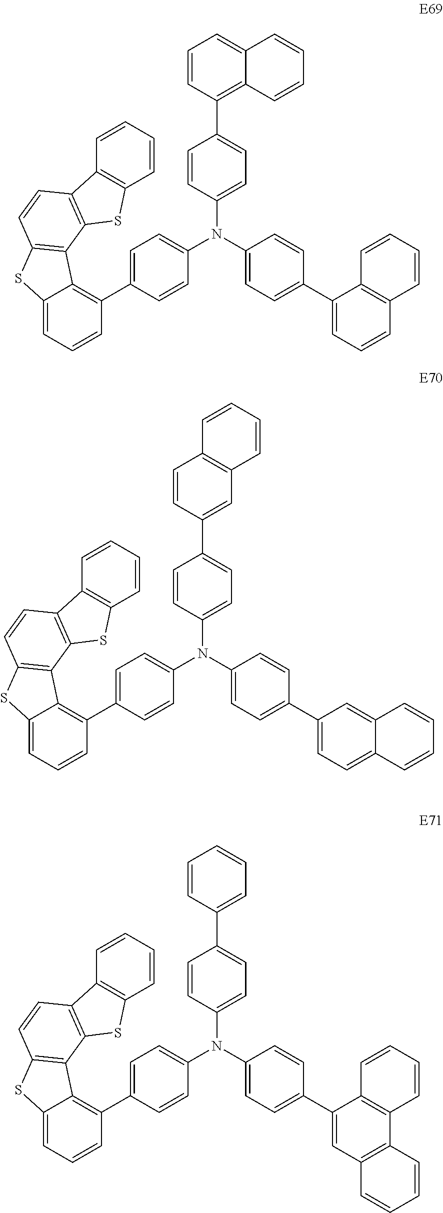 [SMILES: c1ccc2c(-c3ccc(N(c4ccc(-c5cccc6ccccc56)cc4)c4ccc(-c5cccc6sc7ccc8c9ccccc9sc8c7c56)cc4)cc3)cccc2c1 ; c1ccc2cc(-c3ccc(N(c4ccc(-c5ccc6ccccc6c5)cc4)c4ccc(-c5cccc6sc7ccc8c9ccccc9sc8c7c56)cc4)cc3)ccc2c1 ; c1ccc(-c2ccc(N(c3ccc(-c4cc5ccccc5c5ccccc45)cc3)c3ccc(-c4cccc5sc6ccc7c8ccccc8sc7c6c45)cc3)cc2)cc1]

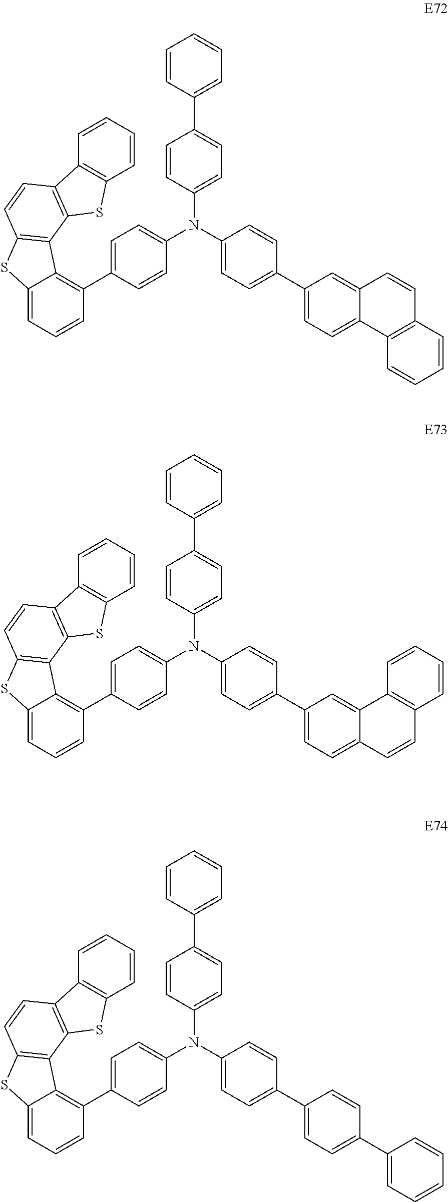 [SMILES: c1ccc(-c2ccc(N(c3ccc(-c4ccc5c(ccc6ccccc65)c4)cc3)c3ccc(-c4cccc5sc6ccc7c8ccccc8sc7c6c45)cc3)cc2)cc1 ; c1ccc(-c2ccc(N(c3ccc(-c4ccc5ccc6ccccc6c5c4)cc3)c3ccc(-c4cccc5sc6ccc7c8ccccc8sc7c6c45)cc3)cc2)cc1 ; c1ccc(-c2ccc(-c3ccc(N(c4ccc(-c5ccccc5)cc4)c4ccc(-c5cccc6sc7ccc8c9ccccc9sc8c7c56)cc4)cc3)cc2)cc1]

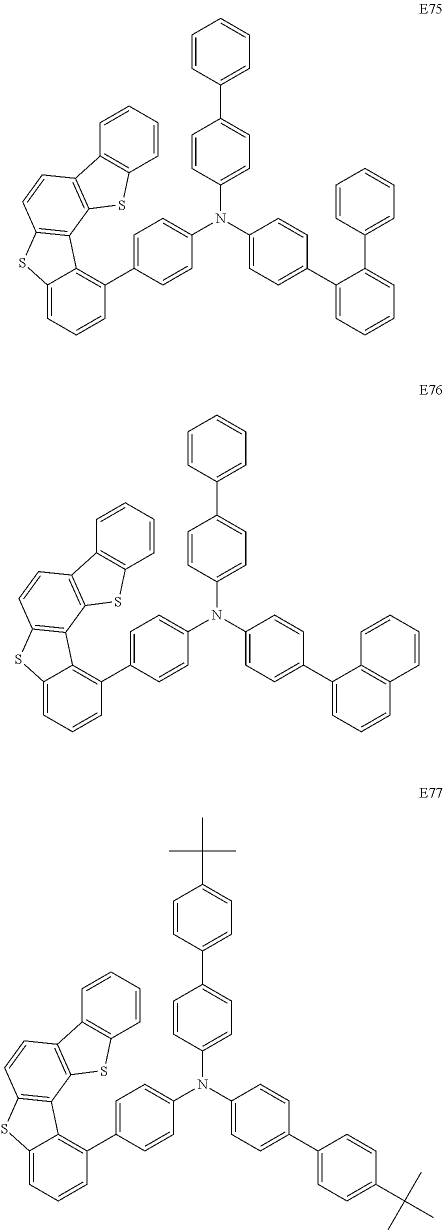 [SMILES: c1ccc(-c2ccc(N(c3ccc(-c4ccccc4-c4ccccc4)cc3)c3ccc(-c4cccc5sc6ccc7c8ccccc8sc7c6c45)cc3)cc2)cc1 ; c1ccc(-c2ccc(N(c3ccc(-c4cccc5ccccc45)cc3)c3ccc(-c4cccc5sc6ccc7c8ccccc8sc7c6c45)cc3)cc2)cc1 ; CC(C)(C)c1ccc(-c2ccc(N(c3ccc(-c4ccc(C(C)(C)C)cc4)cc3)c3ccc(-c4cccc5sc6ccc7c8ccccc8sc7c6c45)cc3)cc2)cc1]

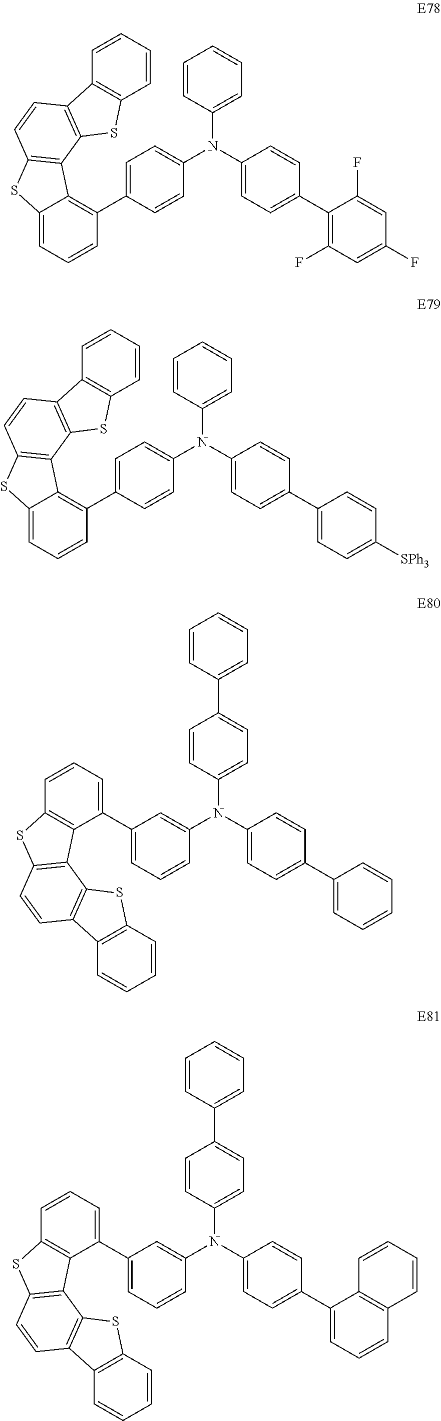

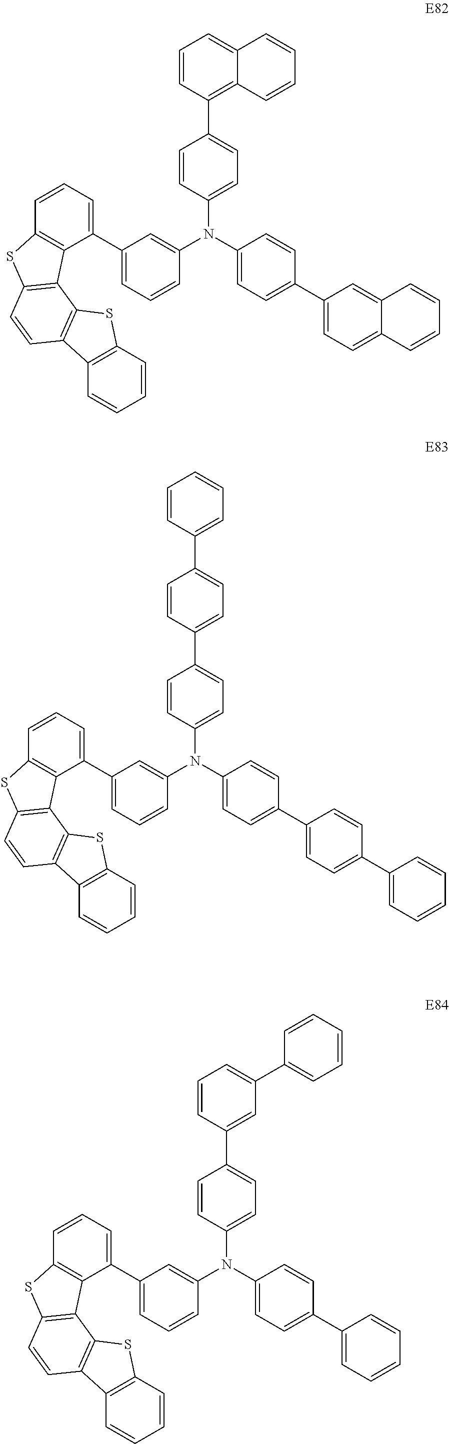 [SMILES: c1cc(-c2cccc3sc4ccc5c6ccccc6sc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6sc7ccc8c9ccccc9sc8c7c56)c4)cc3)cc2)cc1 ; c1ccc(-c2ccc(N(c3ccc(-c4cccc(-c5ccccc5)c4)cc3)c3cccc(-c4cccc5sc6ccc7c8ccccc8sc7c6c45)c3)cc2)cc1]

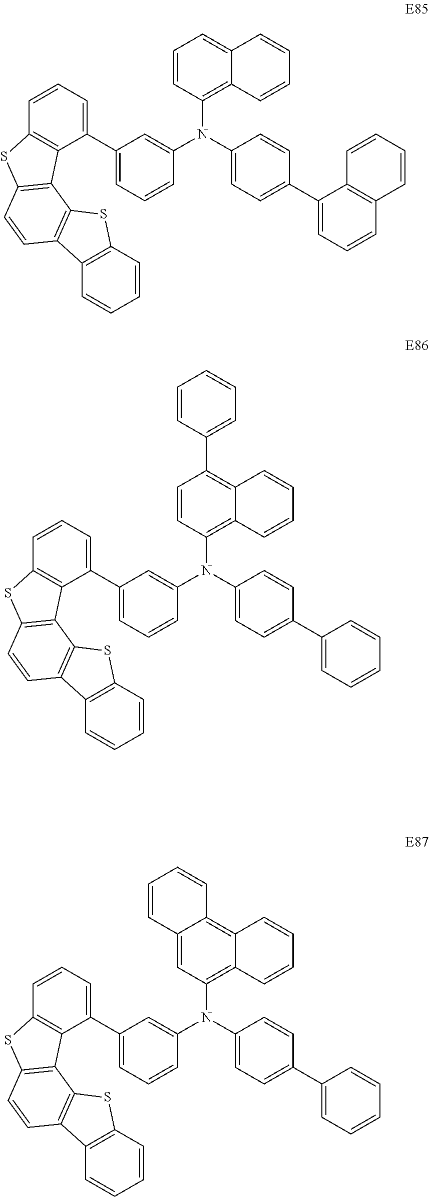 [SMILES: c1cc(-c2cccc3sc4ccc5c6ccccc6sc5c4c23)cc(N(c2ccc(-c3cccc4ccccc34)cc2)c2cccc3ccccc23)c1 ; c1ccc(-c2ccc(N(c3cccc(-c4cccc5sc6ccc7c8ccccc8sc7c6c45)c3)c3ccc(-c4ccccc4)c4ccccc34)cc2)cc1 ; c1ccc(-c2ccc(N(c3cccc(-c4cccc5sc6ccc7c8ccccc8sc7c6c45)c3)c3cc4ccccc4c4ccccc34)cc2)cc1]

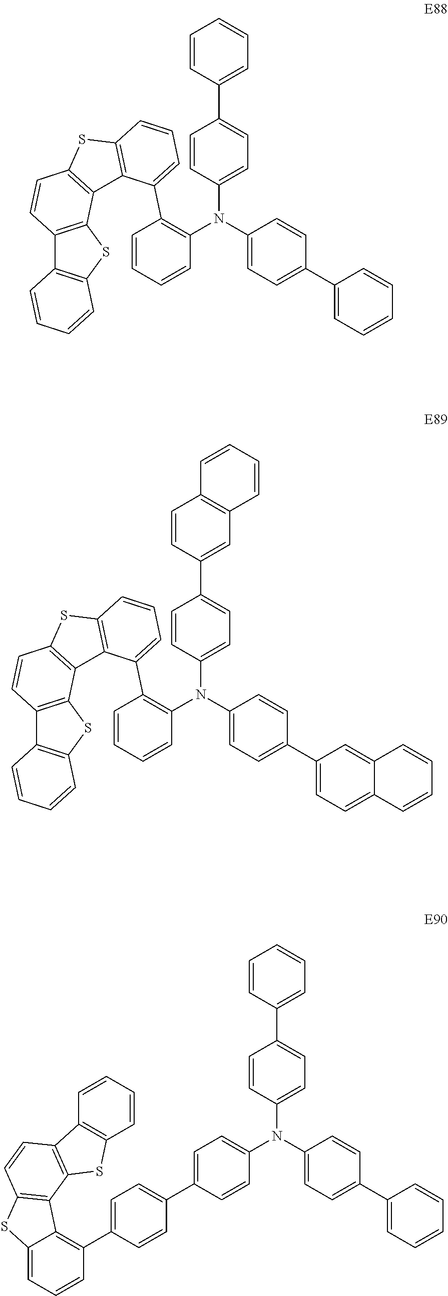 [SMILES: c1ccc(-c2ccc(N(c3ccc(-c4ccccc4)cc3)c3ccccc3-c3cccc4sc5ccc6c7ccccc7sc6c5c34)cc2)cc1 ; c1ccc(N(c2ccc(-c3ccc4ccccc4c3)cc2)c2ccc(-c3ccc4ccccc4c3)cc2)c(-c2cccc3sc4ccc5c6ccccc6sc5c4c23)c1 ; c1ccc(-c2ccc(N(c3ccc(-c4ccccc4)cc3)c3ccc(-c4ccc(-c5cccc6sc7ccc8c9ccccc9sc8c7c56)cc4)cc3)cc2)cc1]

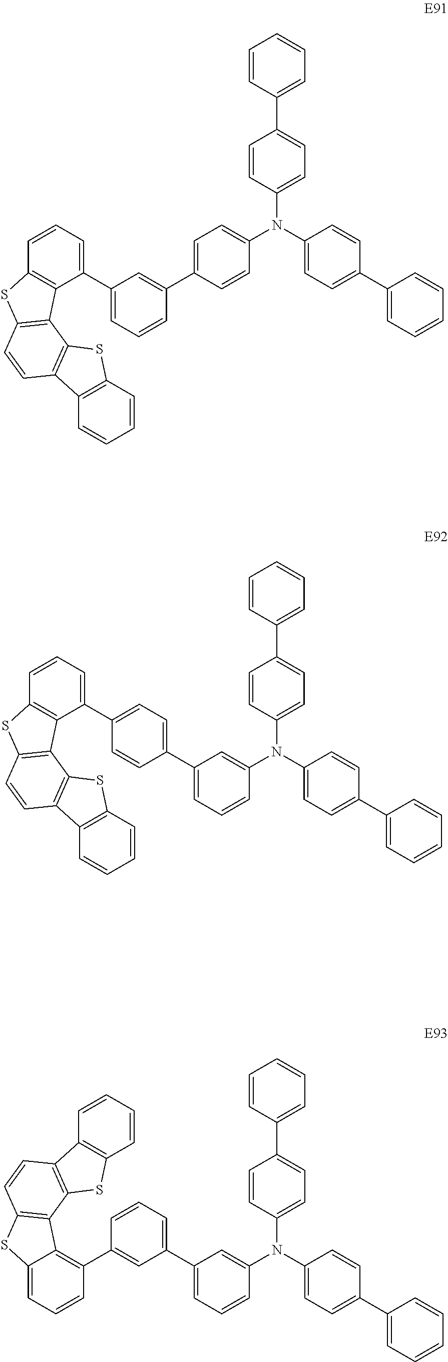 [SMILES: c1ccc(-c2ccc(N(c3ccc(-c4ccccc4)cc3)c3ccc(-c4cccc(-c5cccc6sc7ccc8c9ccccc9sc8c7c56)c4)cc3)cc2)cc1 ; c1ccc(-c2ccc(N(c3ccc(-c4ccccc4)cc3)c3cccc(-c4ccc(-c5cccc6sc7ccc8c9ccccc9sc8c7c56)cc4)c3)cc2)cc1 ; c1ccc(-c2ccc(N(c3ccc(-c4ccccc4)cc3)c3cccc(-c4cccc(-c5cccc6sc7ccc8c9ccccc9sc8c7c56)c4)c3)cc2)cc1]

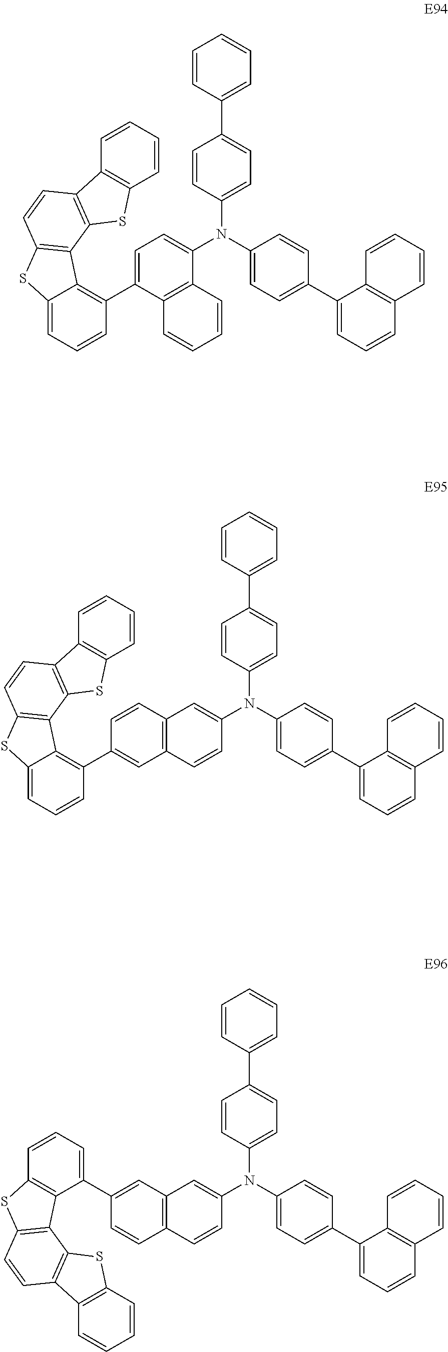 [SMILES: c1ccc(-c2ccc(N(c3ccc(-c4cccc5ccccc45)cc3)c3ccc(-c4cccc5sc6ccc7c8ccccc8sc7c6c45)c4ccccc34)cc2)cc1 ; c1ccc(-c2ccc(N(c3ccc(-c4cccc5ccccc45)cc3)c3ccc4cc(-c5cccc6sc7ccc8c9ccccc9sc8c7c56)ccc4c3)cc2)cc1 ; c1ccc(-c2ccc(N(c3ccc(-c4cccc5ccccc45)cc3)c3ccc4ccc(-c5cccc6sc7ccc8c9ccccc9sc8c7c56)cc4c3)cc2)cc1]

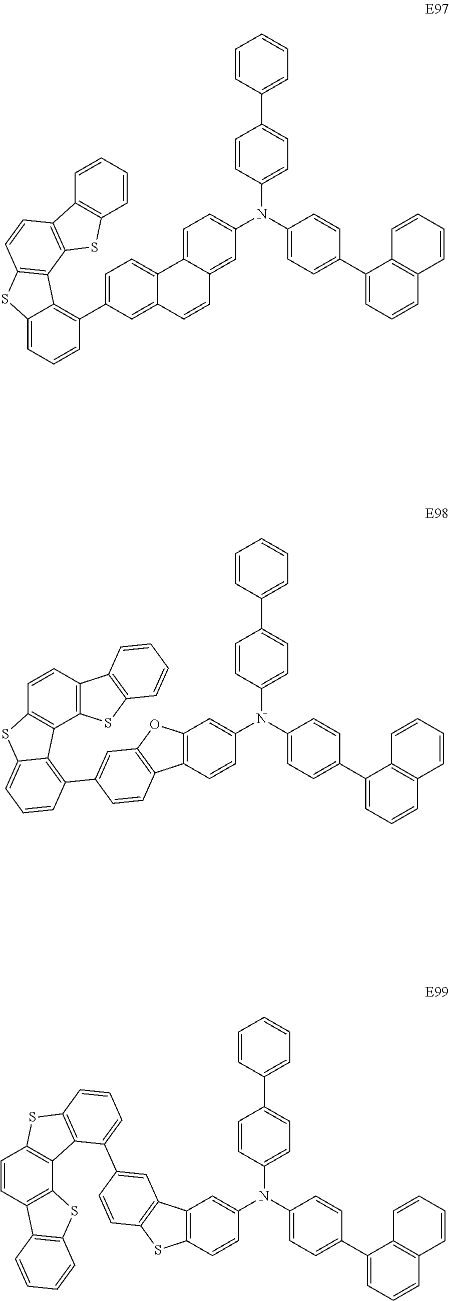 [SMILES: c1ccc(-c2ccc(N(c3ccc(-c4cccc5ccccc45)cc3)c3ccc4c(ccc5cc(-c6cccc7sc8ccc9c%10ccccc%10sc9c8c67)ccc54)c3)cc2)cc1 ; c1ccc(-c2ccc(N(c3ccc(-c4cccc5ccccc45)cc3)c3ccc4c(c3)oc3cc(-c5cccc6sc7ccc8c9ccccc9sc8c7c56)ccc34)cc2)cc1 ; c1ccc(-c2ccc(N(c3ccc(-c4cccc5ccccc45)cc3)c3ccc4sc5ccc(-c6cccc7sc8ccc9c%10ccccc%10sc9c8c67)cc5c4c3)cc2)cc1]

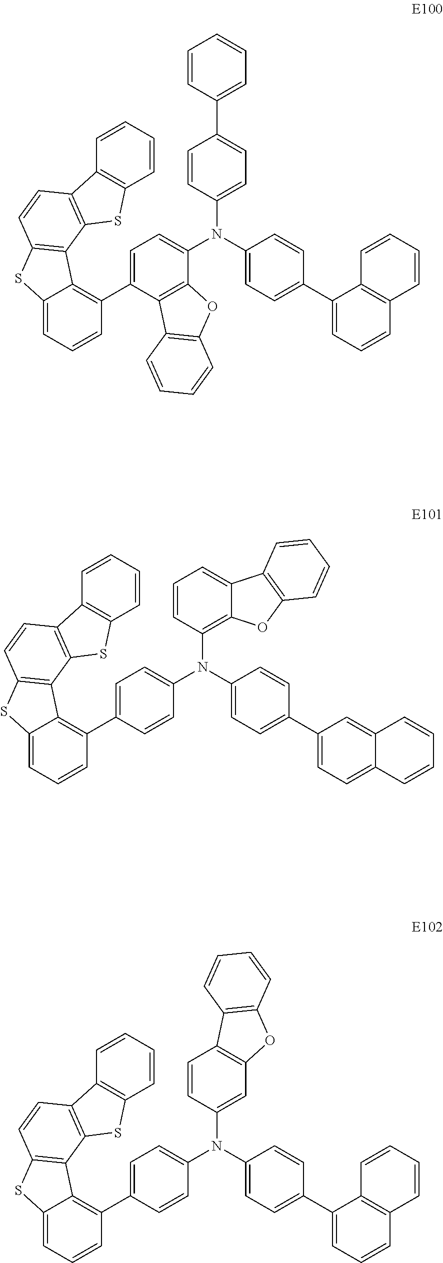 [SMILES: c1ccc(-c2ccc(N(c3ccc(-c4cccc5ccccc45)cc3)c3ccc(-c4cccc5sc6ccc7c8ccccc8sc7c6c45)c4c3oc3ccccc34)cc2)cc1 ; c1ccc2cc(-c3ccc(N(c4ccc(-c5cccc6sc7ccc8c9ccccc9sc8c7c56)cc4)c4cccc5c4oc4ccccc45)cc3)ccc2c1 ; c1ccc2c(-c3ccc(N(c4ccc(-c5cccc6sc7ccc8c9ccccc9sc8c7c56)cc4)c4ccc5c(c4)oc4ccccc45)cc3)cccc2c1]

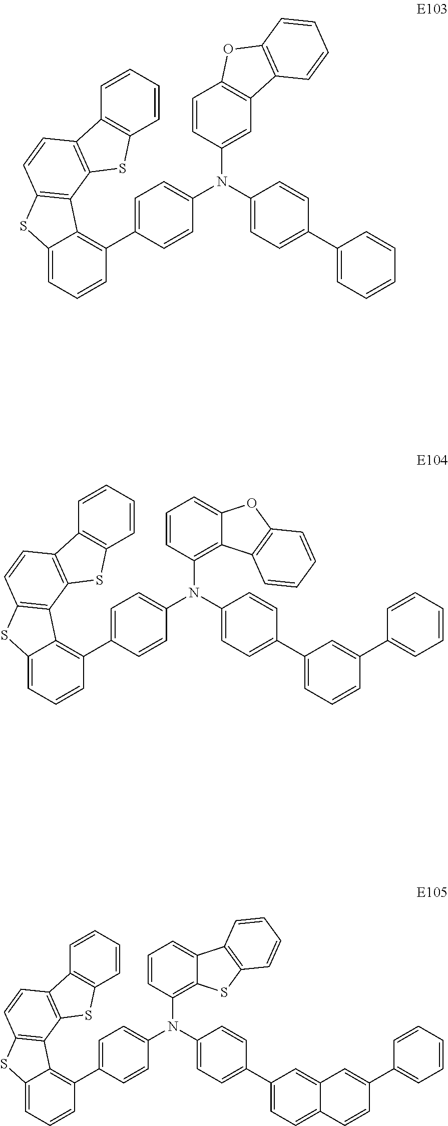 [SMILES: c1ccc(-c2ccc(N(c3ccc(-c4cccc5sc6ccc7c8ccccc8sc7c6c45)cc3)c3ccc4oc5ccccc5c4c3)cc2)cc1 ; c1ccc(-c2cccc(-c3ccc(N(c4ccc(-c5cccc6sc7ccc8c9ccccc9sc8c7c56)cc4)c4cccc5oc6ccccc6c45)cc3)c2)cc1 ; c1ccc(-c2ccc3ccc(-c4ccc(N(c5ccc(-c6cccc7sc8ccc9c%10ccccc%10sc9c8c67)cc5)c5cccc6c5sc5ccccc56)cc4)cc3c2)cc1]

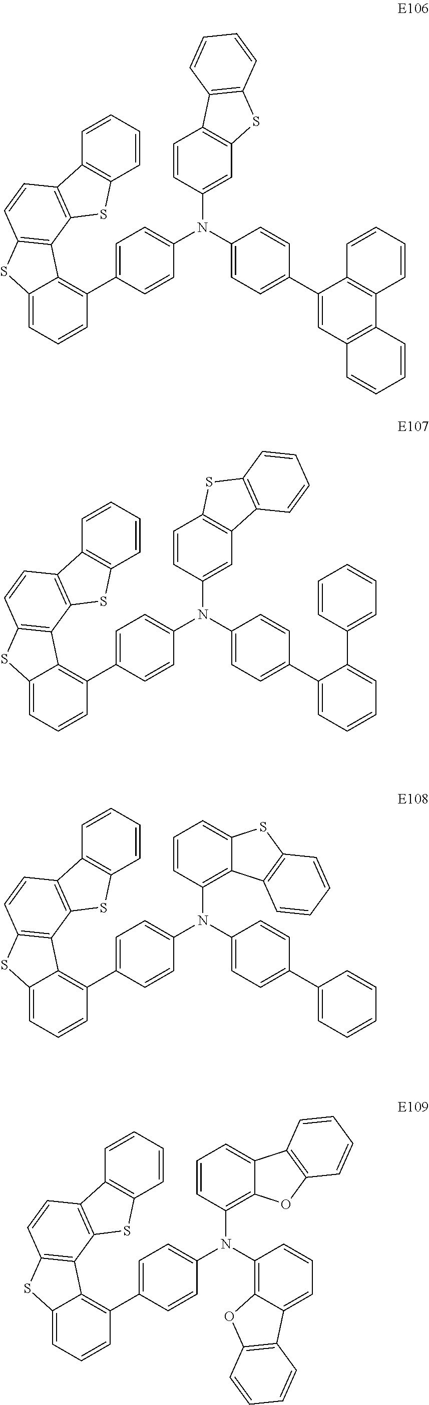 [SMILES: c1ccc2c(c1)cc(-c1ccc(N(c3ccc(-c4cccc5sc6ccc7c8ccccc8sc7c6c45)cc3)c3ccc4c(c3)sc3ccccc34)cc1)c1ccccc12 ; c1ccc(-c2ccccc2-c2ccc(N(c3ccc(-c4cccc5sc6ccc7c8ccccc8sc7c6c45)cc3)c3ccc4sc5ccccc5c4c3)cc2)cc1 ; c1ccc(-c2ccc(N(c3ccc(-c4cccc5sc6ccc7c8ccccc8sc7c6c45)cc3)c3cccc4sc5ccccc5c34)cc2)cc1 ; c1ccc2c(c1)oc1c(N(c3ccc(-c4cccc5sc6ccc7c8ccccc8sc7c6c45)cc3)c3cccc4c3oc3ccccc34)cccc12]

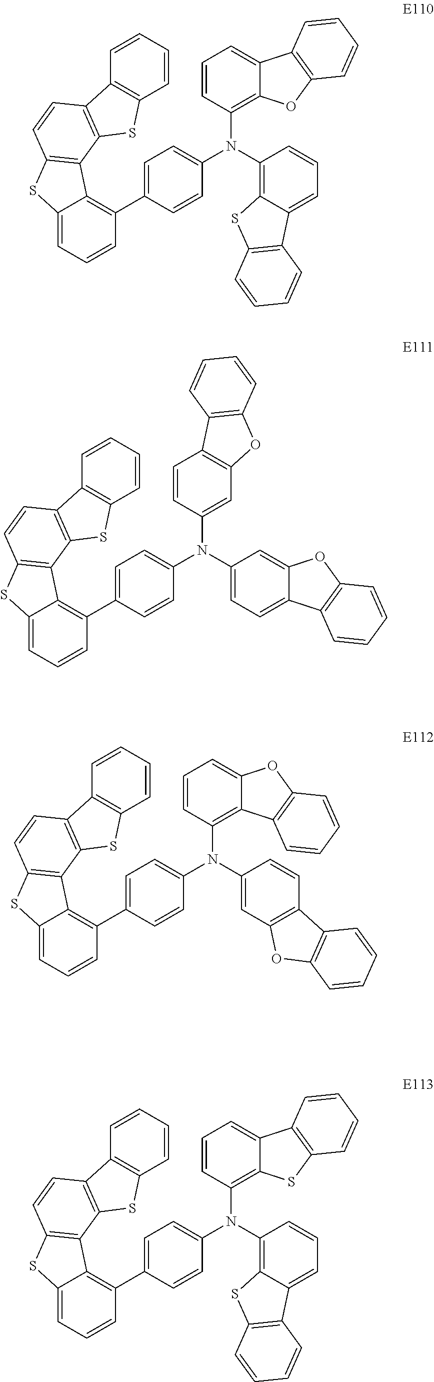 [SMILES: c1ccc2c(c1)oc1c(N(c3ccc(-c4cccc5sc6ccc7c8ccccc8sc7c6c45)cc3)c3cccc4c3sc3ccccc34)cccc12 ; c1ccc2c(c1)oc1cc(N(c3ccc(-c4cccc5sc6ccc7c8ccccc8sc7c6c45)cc3)c3ccc4c(c3)oc3ccccc34)ccc12 ; c1ccc2c(c1)oc1cc(N(c3ccc(-c4cccc5sc6ccc7c8ccccc8sc7c6c45)cc3)c3cccc4oc5ccccc5c34)ccc12 ; c1ccc2c(c1)sc1c(N(c3ccc(-c4cccc5sc6ccc7c8ccccc8sc7c6c45)cc3)c3cccc4c3sc3ccccc34)cccc12]

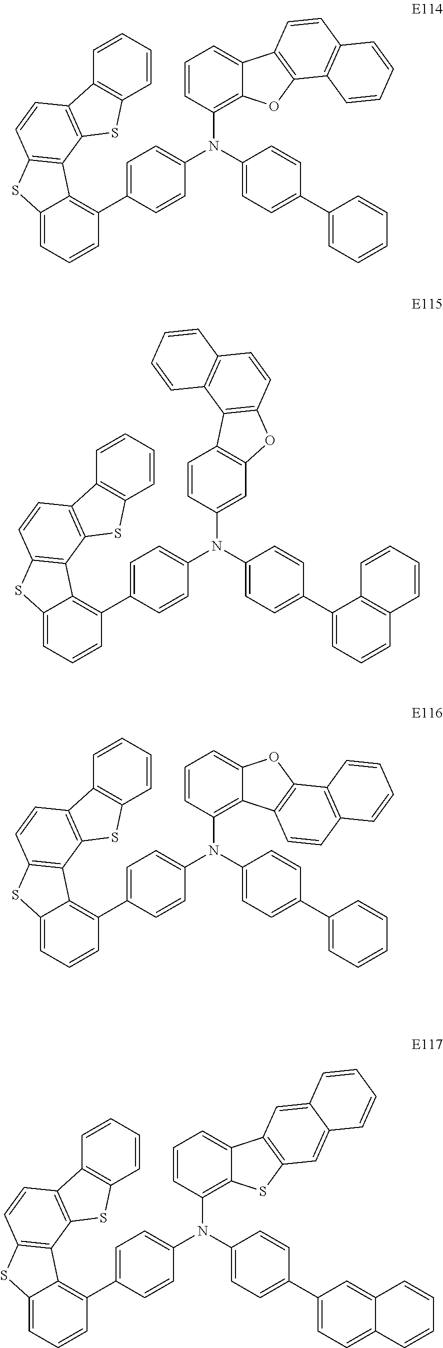 [SMILES: c1ccc(-c2ccc(N(c3ccc(-c4cccc5sc6ccc7c8ccccc8sc7c6c45)cc3)c3cccc4c3oc3c5ccccc5ccc43)cc2)cc1 ; c1ccc2c(-c3ccc(N(c4ccc(-c5cccc6sc7ccc8c9ccccc9sc8c7c56)cc4)c4ccc5c(c4)oc4ccc6ccccc6c45)cc3)cccc2c1 ; c1ccc(-c2ccc(N(c3ccc(-c4cccc5sc6ccc7c8ccccc8sc7c6c45)cc3)c3cccc4oc5c6ccccc6ccc5c34)cc2)cc1 ; c1ccc2cc(-c3ccc(N(c4ccc(-c5cccc6sc7ccc8c9ccccc9sc8c7c56)cc4)c4cccc5c4sc4cc6ccccc6cc45)cc3)ccc2c1]

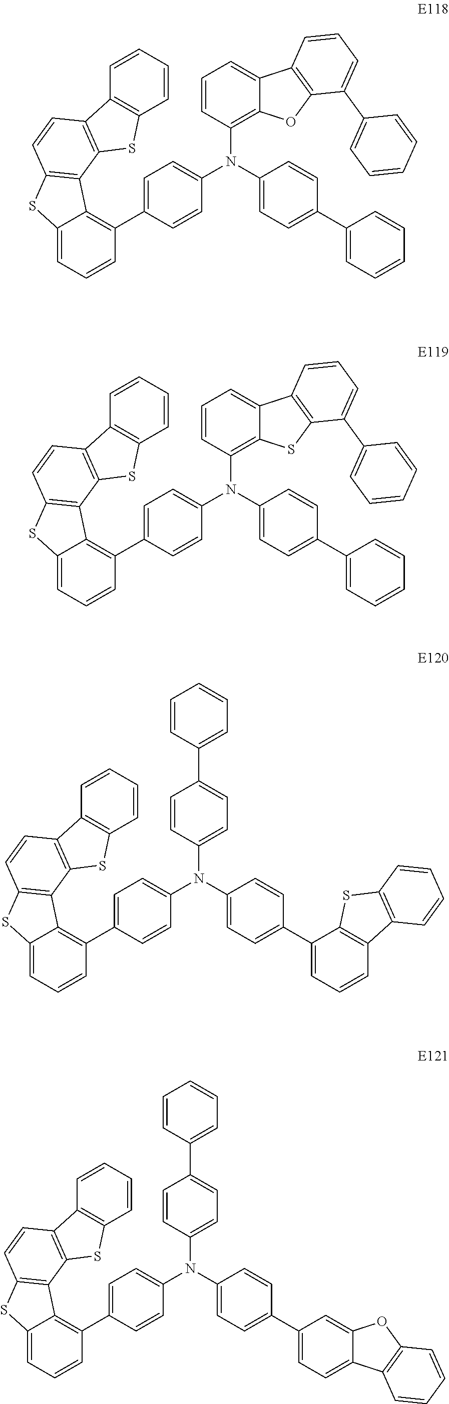 [SMILES: c1ccc(-c2ccc(N(c3ccc(-c4cccc5sc6ccc7c8ccccc8sc7c6c45)cc3)c3cccc4c3oc3c(-c5ccccc5)cccc34)cc2)cc1 ; c1ccc(-c2ccc(N(c3ccc(-c4cccc5sc6ccc7c8ccccc8sc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5sc6ccc7c8ccccc8sc7c6c45)cc3)cc2)cc1 ; c1ccc(-c2ccc(N(c3ccc(-c4ccc5c(c4)oc4ccccc45)cc3)c3ccc(-c4cccc5sc6ccc7c8ccccc8sc7c6c45)cc3)cc2)cc1]

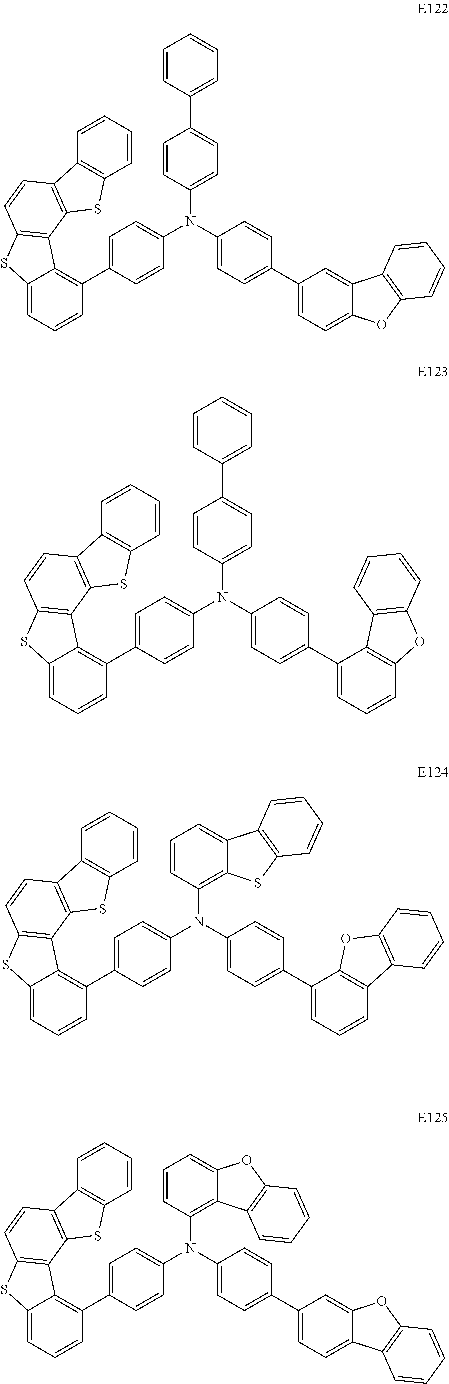 [SMILES: c1ccc(-c2ccc(N(c3ccc(-c4ccc5oc6ccccc6c5c4)cc3)c3ccc(-c4cccc5sc6ccc7c8ccccc8sc7c6c45)cc3)cc2)cc1 ; c1ccc(-c2ccc(N(c3ccc(-c4cccc5oc6ccccc6c45)cc3)c3ccc(-c4cccc5sc6ccc7c8ccccc8sc7c6c45)cc3)cc2)cc1 ; c1ccc2c(c1)oc1c(-c3ccc(N(c4ccc(-c5cccc6sc7ccc8c9ccccc9sc8c7c56)cc4)c4cccc5c4sc4ccccc45)cc3)cccc12 ; c1ccc2c(c1)oc1cc(-c3ccc(N(c4ccc(-c5cccc6sc7ccc8c9ccccc9sc8c7c56)cc4)c4cccc5oc6ccccc6c45)cc3)ccc12]

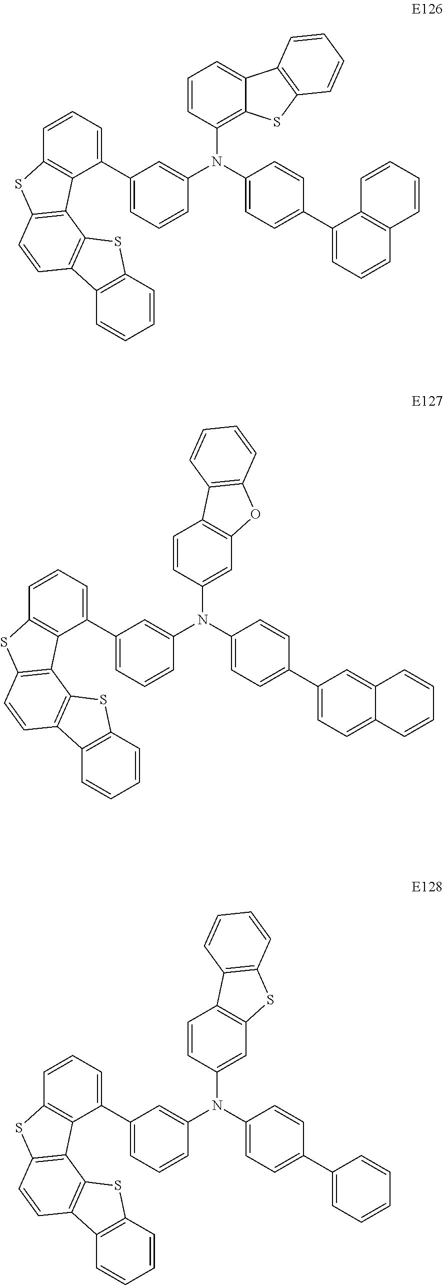 [SMILES: c1cc(-c2cccc3sc4ccc5c6ccccc6sc5c4c23)cc(N(c2ccc(-c3cccc4ccccc34)cc2)c2cccc3c2sc2ccccc23)c1 ; c1cc(-c2cccc3sc4ccc5c6ccccc6sc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5sc6ccc7c8ccccc8sc7c6c45)c3)c3ccc4c(c3)sc3ccccc34)cc2)cc1]

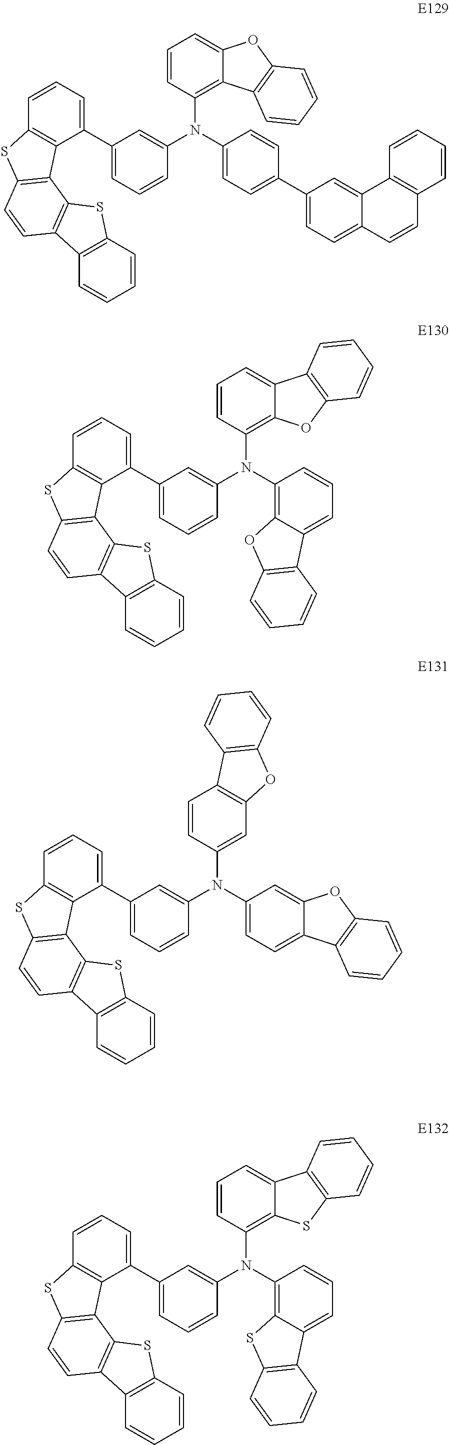 [SMILES: c1cc(-c2cccc3sc4ccc5c6ccccc6sc5c4c23)cc(N(c2ccc(-c3ccc4ccc5ccccc5c4c3)cc2)c2cccc3oc4ccccc4c23)c1 ; c1cc(-c2cccc3sc4ccc5c6ccccc6sc5c4c23)cc(N(c2cccc3c2oc2ccccc23)c2cccc3c2oc2ccccc23)c1 ; c1cc(-c2cccc3sc4ccc5c6ccccc6sc5c4c23)cc(N(c2ccc3c(c2)oc2ccccc23)c2ccc3c(c2)oc2ccccc23)c1 ; c1cc(-c2cccc3sc4ccc5c6ccccc6sc5c4c23)cc(N(c2cccc3c2sc2ccccc23)c2cccc3c2sc2ccccc23)c1]

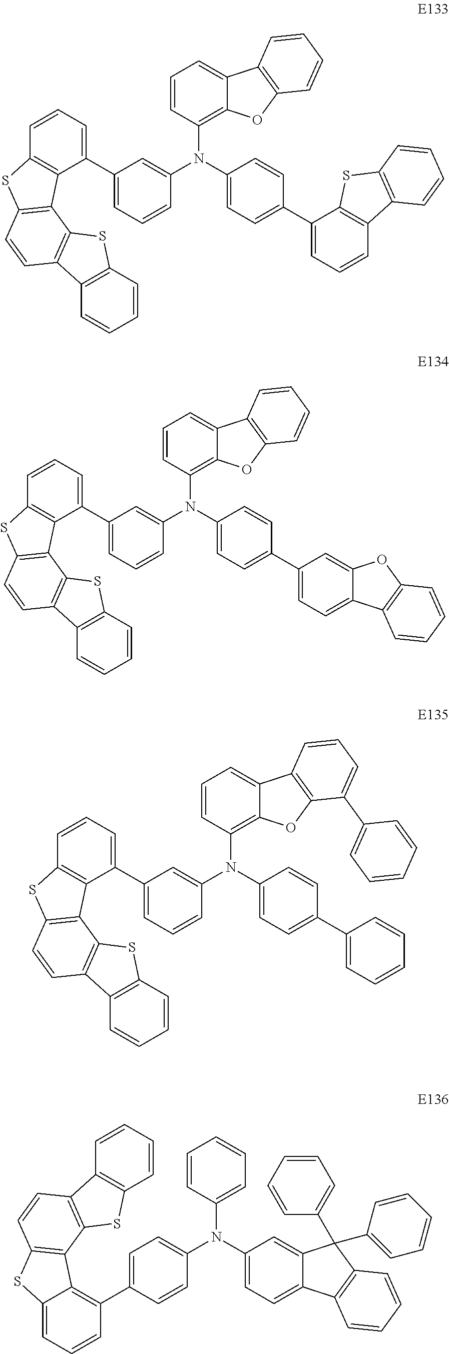 [SMILES: c1cc(-c2cccc3sc4ccc5c6ccccc6sc5c4c23)cc(N(c2ccc(-c3cccc4c3sc3ccccc34)cc2)c2cccc3c2oc2ccccc23)c1 ; c1cc(-c2cccc3sc4ccc5c6ccccc6sc5c4c23)cc(N(c2ccc(-c3ccc4c(c3)oc3ccccc34)cc2)c2cccc3c2oc2ccccc23)c1 ; c1ccc(-c2ccc(N(c3cccc(-c4cccc5sc6ccc7c8ccccc8sc7c6c45)c3)c3cccc4c3oc3c(-c5ccccc5)cccc34)cc2)cc1 ; c1ccc(N(c2ccc(-c3cccc4sc5ccc6c7ccccc7sc6c5c34)cc2)c2ccc3c(c2)C(c2ccccc2)(c2ccccc2)c2ccccc2-3)cc1]

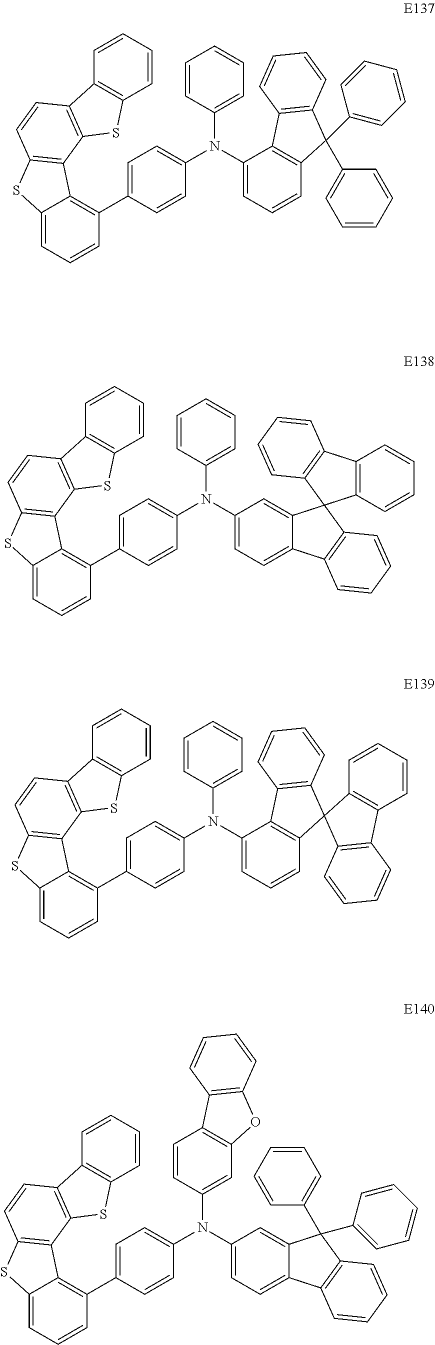 [SMILES: c1ccc(N(c2ccc(-c3cccc4sc5ccc6c7ccccc7sc6c5c34)cc2)c2cccc3c2-c2ccccc2C3(c2ccccc2)c2ccccc2)cc1 ; c1ccc(N(c2ccc(-c3cccc4sc5ccc6c7ccccc7sc6c5c34)cc2)c2ccc3c(c2)C2(c4ccccc4-c4ccccc42)c2ccccc2-3)cc1 ; c1ccc(N(c2ccc(-c3cccc4sc5ccc6c7ccccc7sc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6sc7ccc8c9ccccc9sc8c7c56)cc4)c4ccc5c(c4)oc4ccccc45)cc32)cc1]

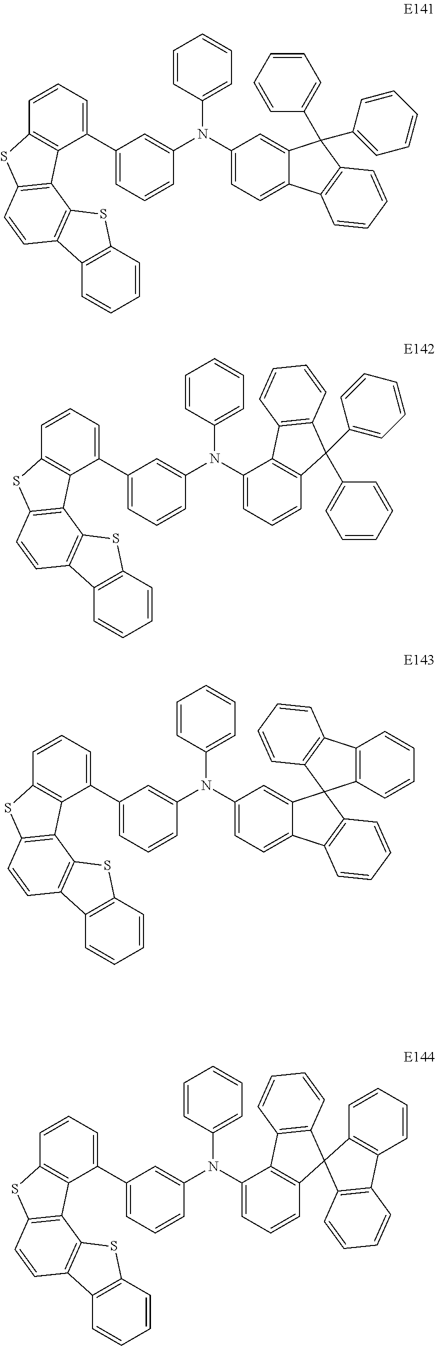 [SMILES: c1ccc(N(c2cccc(-c3cccc4sc5ccc6c7ccccc7sc6c5c34)c2)c2ccc3c(c2)C(c2ccccc2)(c2ccccc2)c2ccccc2-3)cc1 ; c1ccc(N(c2cccc(-c3cccc4sc5ccc6c7ccccc7sc6c5c34)c2)c2cccc3c2-c2ccccc2C3(c2ccccc2)c2ccccc2)cc1 ; c1ccc(N(c2cccc(-c3cccc4sc5ccc6c7ccccc7sc6c5c34)c2)c2ccc3c(c2)C2(c4ccccc4-c4ccccc42)c2ccccc2-3)cc1 ; c1ccc(N(c2cccc(-c3cccc4sc5ccc6c7ccccc7sc6c5c34)c2)c2cccc3c2-c2ccccc2C32c3ccccc3-c3ccccc32)cc1]

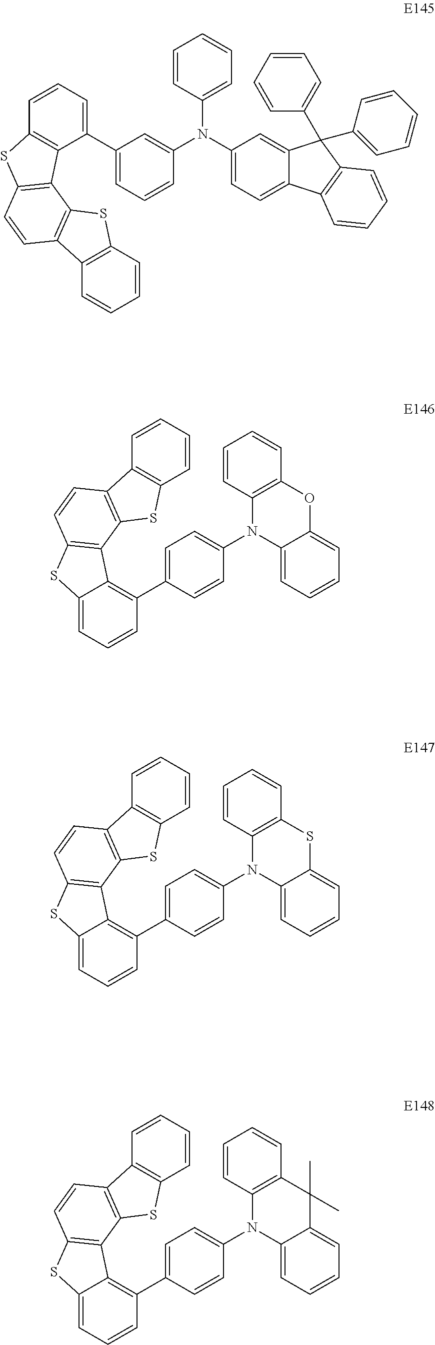 [SMILES: c1ccc(N(c2cccc(-c3cccc4sc5ccc6c7ccccc7sc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3sc4ccc5c6ccccc6sc5c4c23)cc1 ; c1ccc2c(c1)Sc1ccccc1N2c1ccc(-c2cccc3sc4ccc5c6ccccc6sc5c4c23)cc1 ; CC1(C)c2ccccc2N(c2ccc(-c3cccc4sc5ccc6c7ccccc7sc6c5c34)cc2)c2ccccc21]

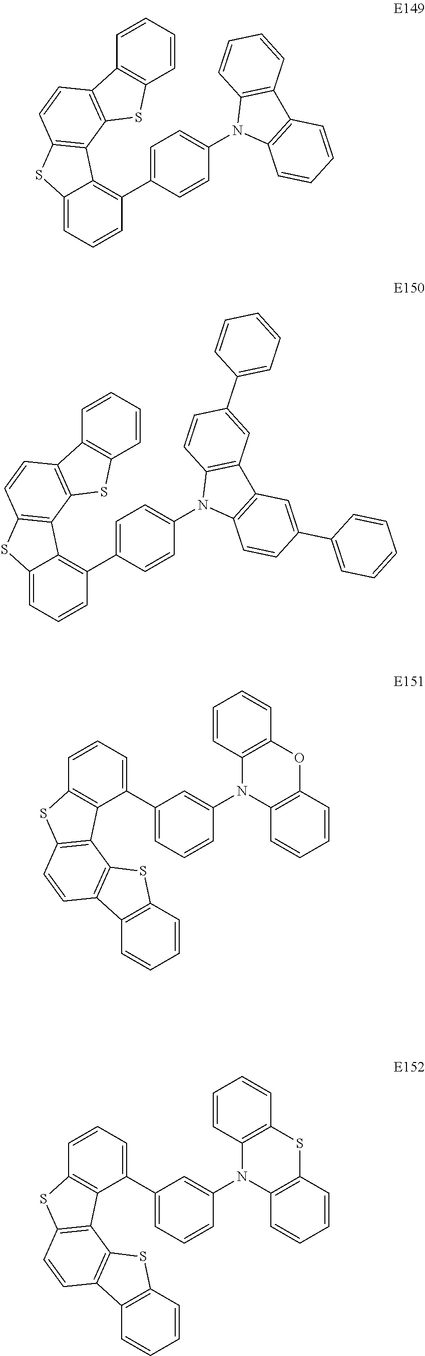 [SMILES: c1ccc2c(c1)sc1c2ccc2sc3cccc(-c4ccc(-n5c6ccccc6c6ccccc65)cc4)c3c21 ; c1ccc(-c2ccc3c(c2)c2cc(-c4ccccc4)ccc2n3-c2ccc(-c3cccc4sc5ccc6c7ccccc7sc6c5c34)cc2)cc1 ; c1cc(-c2cccc3sc4ccc5c6ccccc6sc5c4c23)cc(N2c3ccccc3Oc3ccccc32)c1 ; c1cc(-c2cccc3sc4ccc5c6ccccc6sc5c4c23)cc(N2c3ccccc3Sc3ccccc32)c1]

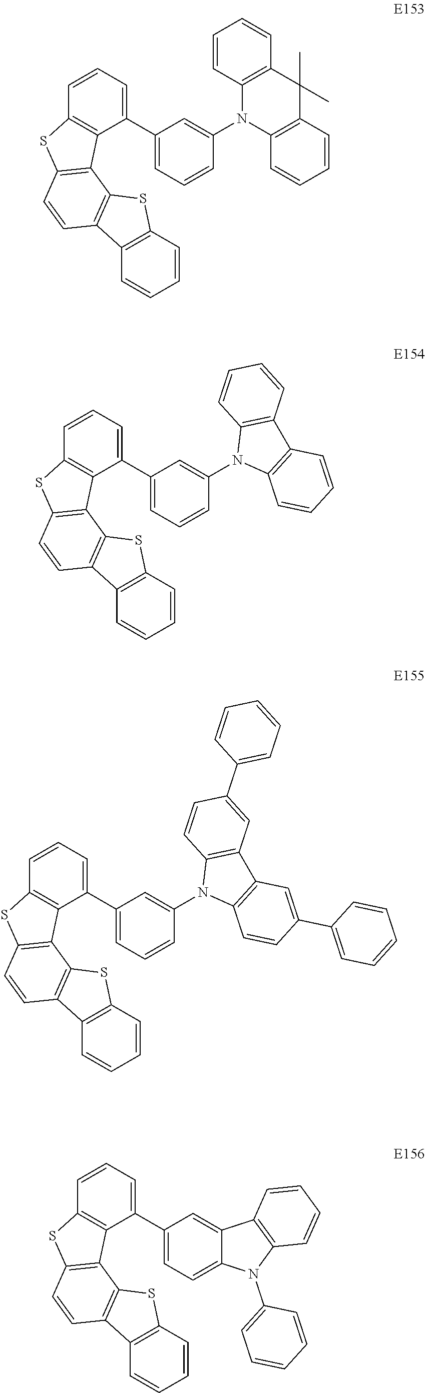 [SMILES: CC1(C)c2ccccc2N(c2cccc(-c3cccc4sc5ccc6c7ccccc7sc6c5c34)c2)c2ccccc21 ; c1cc(-c2cccc3sc4ccc5c6ccccc6sc5c4c23)cc(-n2c3ccccc3c3ccccc32)c1 ; c1ccc(-c2ccc3c(c2)c2cc(-c4ccccc4)ccc2n3-c2cccc(-c3cccc4sc5ccc6c7ccccc7sc6c5c34)c2)cc1 ; c1ccc(-n2c3ccccc3c3cc(-c4cccc5sc6ccc7c8ccccc8sc7c6c45)ccc32)cc1]

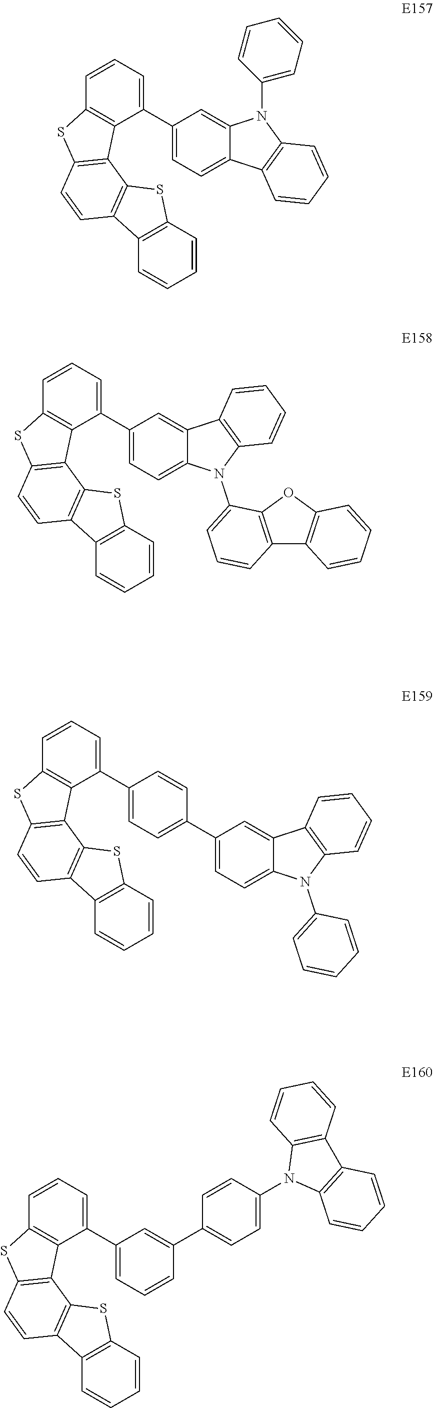 [SMILES: c1ccc(-n2c3ccccc3c3ccc(-c4cccc5sc6ccc7c8ccccc8sc7c6c45)cc32)cc1 ; c1ccc2c(c1)oc1c(-n3c4ccccc4c4cc(-c5cccc6sc7ccc8c9ccccc9sc8c7c56)ccc43)cccc12 ; c1ccc(-n2c3ccccc3c3cc(-c4ccc(-c5cccc6sc7ccc8c9ccccc9sc8c7c56)cc4)ccc32)cc1 ; c1cc(-c2ccc(-n3c4ccccc4c4ccccc43)cc2)cc(-c2cccc3sc4ccc5c6ccccc6sc5c4c23)c1]

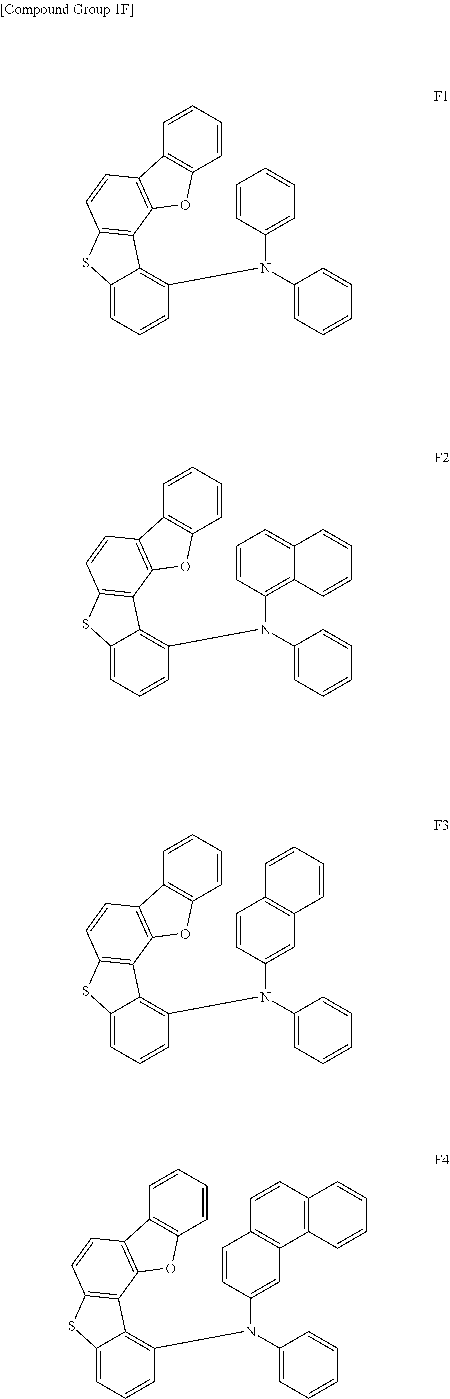 [SMILES: c1ccc(N(c2ccccc2)c2cccc3sc4ccc5c6ccccc6oc5c4c23)cc1 ; c1ccc(N(c2cccc3ccccc23)c2cccc3sc4ccc5c6ccccc6oc5c4c23)cc1 ; c1ccc(N(c2ccc3ccccc3c2)c2cccc3sc4ccc5c6ccccc6oc5c4c23)cc1 ; c1ccc(N(c2ccc3ccc4ccccc4c3c2)c2cccc3sc4ccc5c6ccccc6oc5c4c23)cc1]

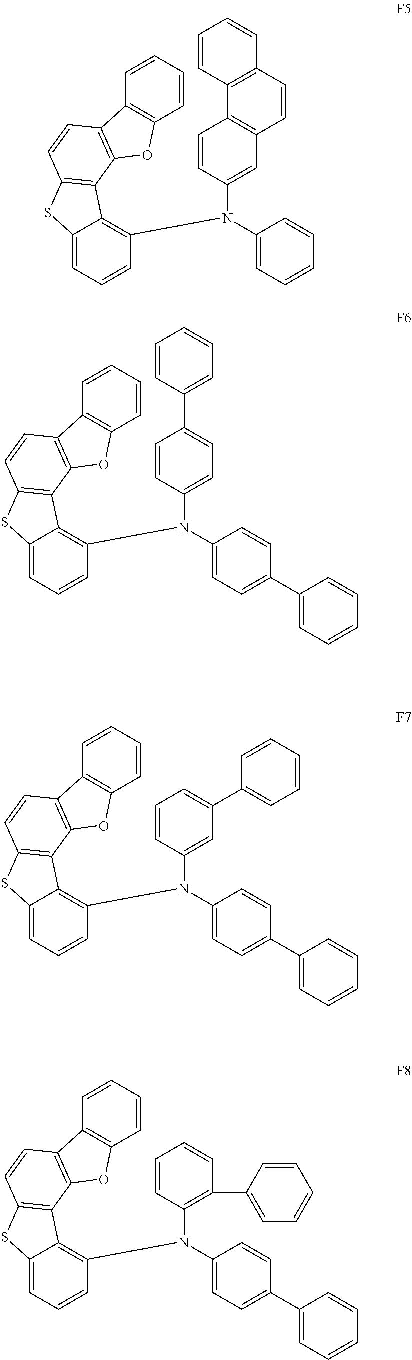 [SMILES: c1ccc(N(c2ccc3c(ccc4ccccc43)c2)c2cccc3sc4ccc5c6ccccc6oc5c4c23)cc1 ; c1ccc(-c2ccc(N(c3ccc(-c4ccccc4)cc3)c3cccc4sc5ccc6c7ccccc7oc6c5c34)cc2)cc1 ; c1ccc(-c2ccc(N(c3cccc(-c4ccccc4)c3)c3cccc4sc5ccc6c7ccccc7oc6c5c34)cc2)cc1 ; c1ccc(-c2ccc(N(c3ccccc3-c3ccccc3)c3cccc4sc5ccc6c7ccccc7oc6c5c34)cc2)cc1]

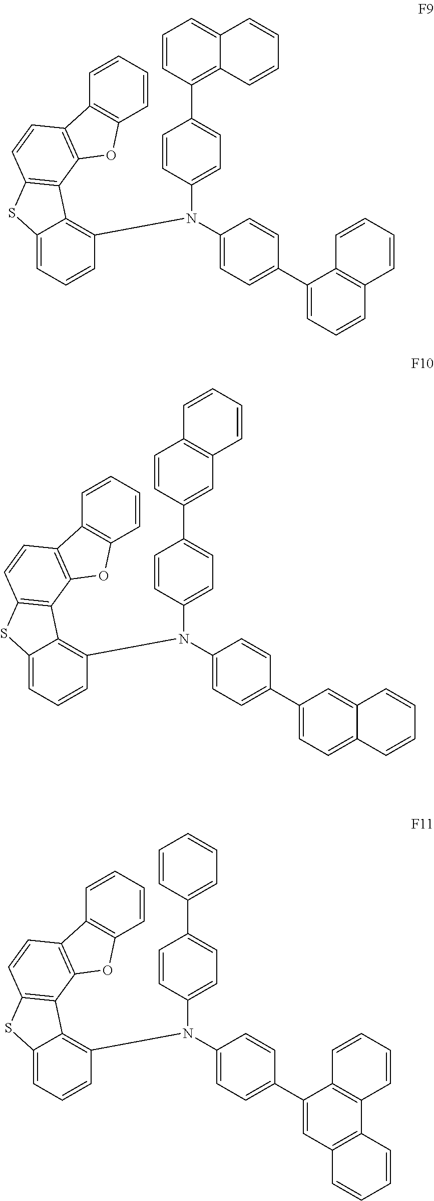 [SMILES: c1ccc2c(-c3ccc(N(c4ccc(-c5cccc6ccccc56)cc4)c4cccc5sc6ccc7c8ccccc8oc7c6c45)cc3)cccc2c1 ; c1ccc2cc(-c3ccc(N(c4ccc(-c5ccc6ccccc6c5)cc4)c4cccc5sc6ccc7c8ccccc8oc7c6c45)cc3)ccc2c1 ; c1ccc(-c2ccc(N(c3ccc(-c4cc5ccccc5c5ccccc45)cc3)c3cccc4sc5ccc6c7ccccc7oc6c5c34)cc2)cc1]

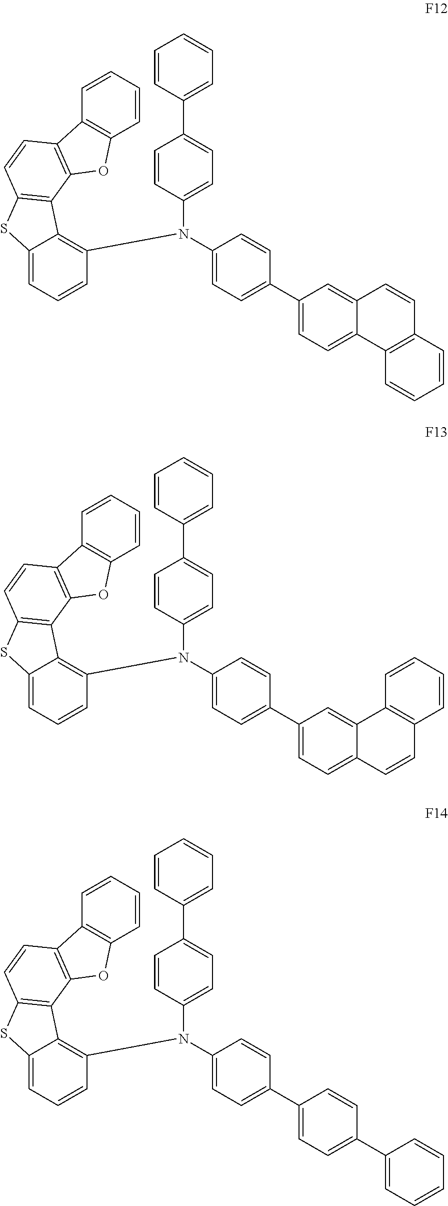 [SMILES: c1ccc(-c2ccc(N(c3ccc(-c4ccc5c(ccc6ccccc65)c4)cc3)c3cccc4sc5ccc6c7ccccc7oc6c5c34)cc2)cc1 ; c1ccc(-c2ccc(N(c3ccc(-c4ccc5ccc6ccccc6c5c4)cc3)c3cccc4sc5ccc6c7ccccc7oc6c5c34)cc2)cc1 ; c1ccc(-c2ccc(-c3ccc(N(c4ccc(-c5ccccc5)cc4)c4cccc5sc6ccc7c8ccccc8oc7c6c45)cc3)cc2)cc1]

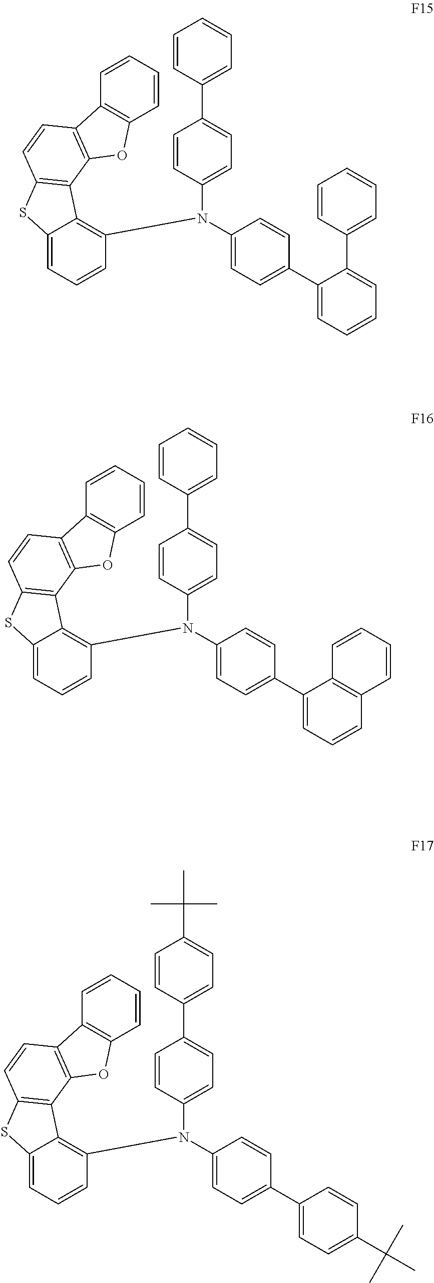 [SMILES: c1ccc(-c2ccc(N(c3ccc(-c4ccccc4-c4ccccc4)cc3)c3cccc4sc5ccc6c7ccccc7oc6c5c34)cc2)cc1 ; c1ccc(-c2ccc(N(c3ccc(-c4cccc5ccccc45)cc3)c3cccc4sc5ccc6c7ccccc7oc6c5c34)cc2)cc1 ; CC(C)(C)c1ccc(-c2ccc(N(c3ccc(-c4ccc(C(C)(C)C)cc4)cc3)c3cccc4sc5ccc6c7ccccc7oc6c5c34)cc2)cc1]

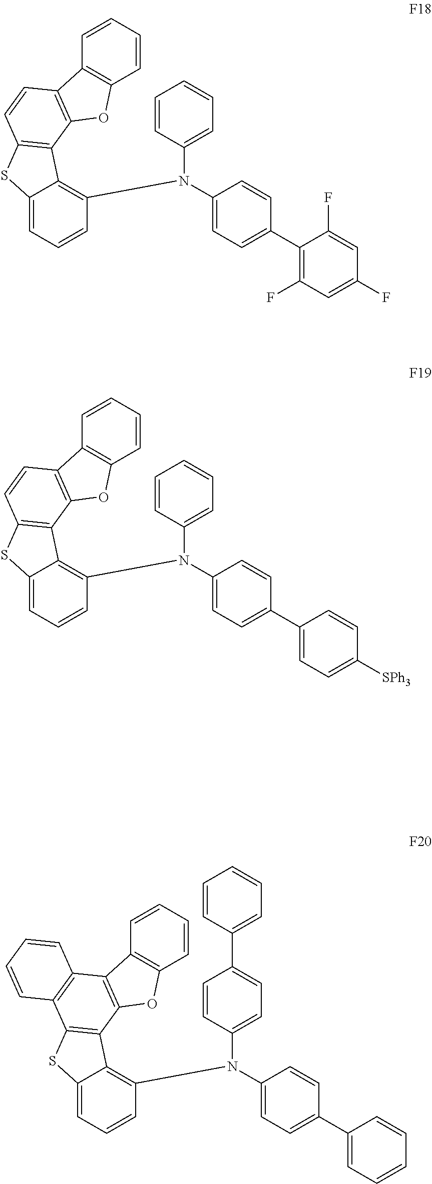

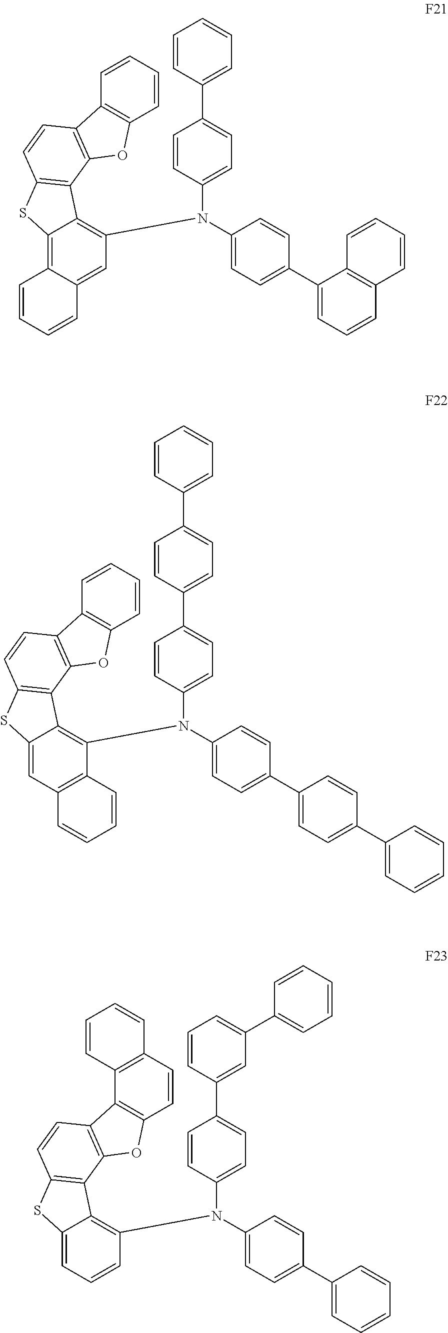 [SMILES: c1ccc(-c2ccc(N(c3ccc(-c4cccc5ccccc45)cc3)c3cc4ccccc4c4sc5ccc6c7ccccc7oc6c5c34)cc2)cc1 ; c1ccc(-c2ccc(-c3ccc(N(c4ccc(-c5ccc(-c6ccccc6)cc5)cc4)c4c5ccccc5cc5sc6ccc7c8ccccc8oc7c6c45)cc3)cc2)cc1 ; c1ccc(-c2ccc(N(c3ccc(-c4cccc(-c5ccccc5)c4)cc3)c3cccc4sc5ccc6c(oc7ccc8ccccc8c76)c5c34)cc2)cc1]

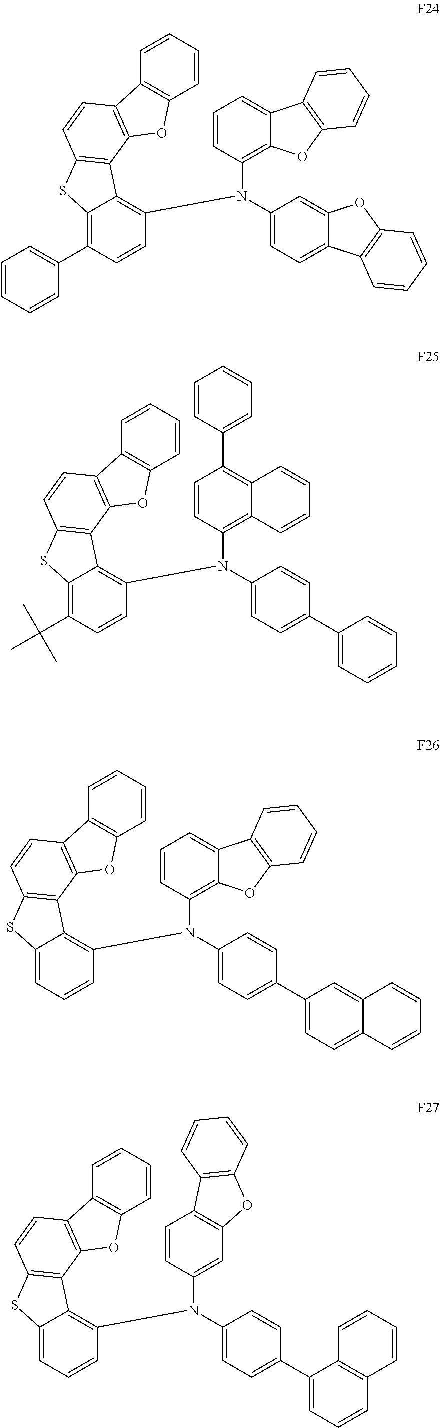 [SMILES: c1ccc(-c2ccc(N(c3ccc4c(c3)oc3ccccc34)c3cccc4c3oc3ccccc34)c3c2sc2ccc4c5ccccc5oc4c23)cc1 ; CC(C)(C)c1ccc(N(c2ccc(-c3ccccc3)cc2)c2ccc(-c3ccccc3)c3ccccc23)c2c1sc1ccc3c4ccccc4oc3c12 ; c1ccc2cc(-c3ccc(N(c4cccc5c4oc4ccccc45)c4cccc5sc6ccc7c8ccccc8oc7c6c45)cc3)ccc2c1 ; c1ccc2c(-c3ccc(N(c4ccc5c(c4)oc4ccccc45)c4cccc5sc6ccc7c8ccccc8oc7c6c45)cc3)cccc2c1]

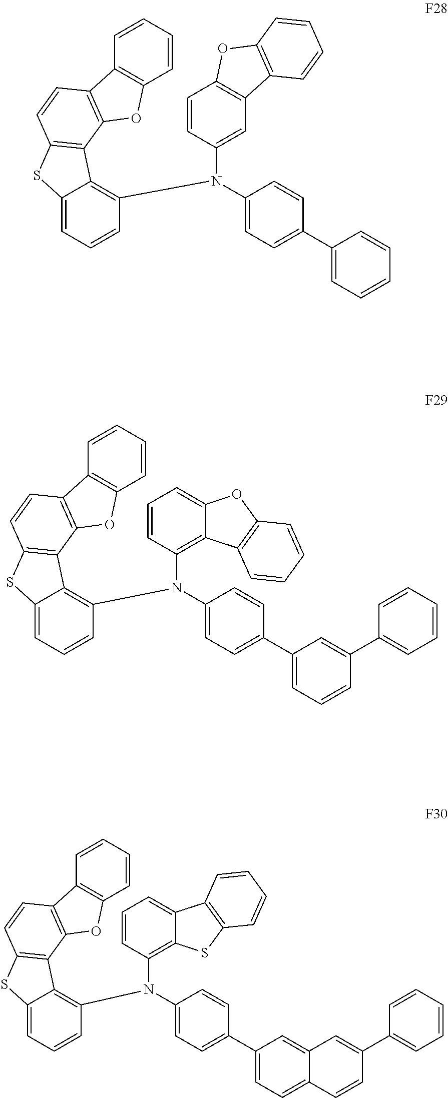 [SMILES: c1ccc(-c2ccc(N(c3ccc4oc5ccccc5c4c3)c3cccc4sc5ccc6c7ccccc7oc6c5c34)cc2)cc1 ; c1ccc(-c2cccc(-c3ccc(N(c4cccc5oc6ccccc6c45)c4cccc5sc6ccc7c8ccccc8oc7c6c45)cc3)c2)cc1 ; c1ccc(-c2ccc3ccc(-c4ccc(N(c5cccc6c5sc5ccccc56)c5cccc6sc7ccc8c9ccccc9oc8c7c56)cc4)cc3c2)cc1]

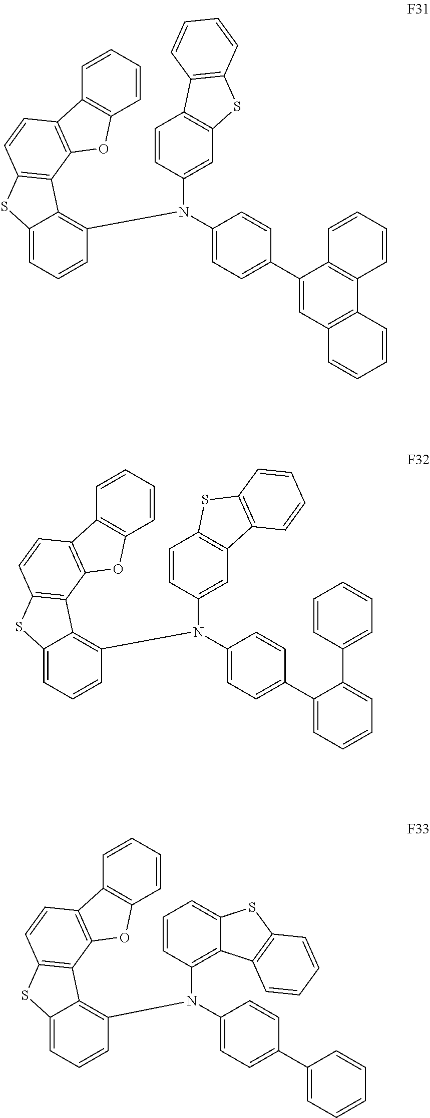 [SMILES: c1ccc2c(c1)cc(-c1ccc(N(c3ccc4c(c3)sc3ccccc34)c3cccc4sc5ccc6c7ccccc7oc6c5c34)cc1)c1ccccc12 ; c1ccc(-c2ccccc2-c2ccc(N(c3ccc4sc5ccccc5c4c3)c3cccc4sc5ccc6c7ccccc7oc6c5c34)cc2)cc1 ; c1ccc(-c2ccc(N(c3cccc4sc5ccccc5c34)c3cccc4sc5ccc6c7ccccc7oc6c5c34)cc2)cc1]

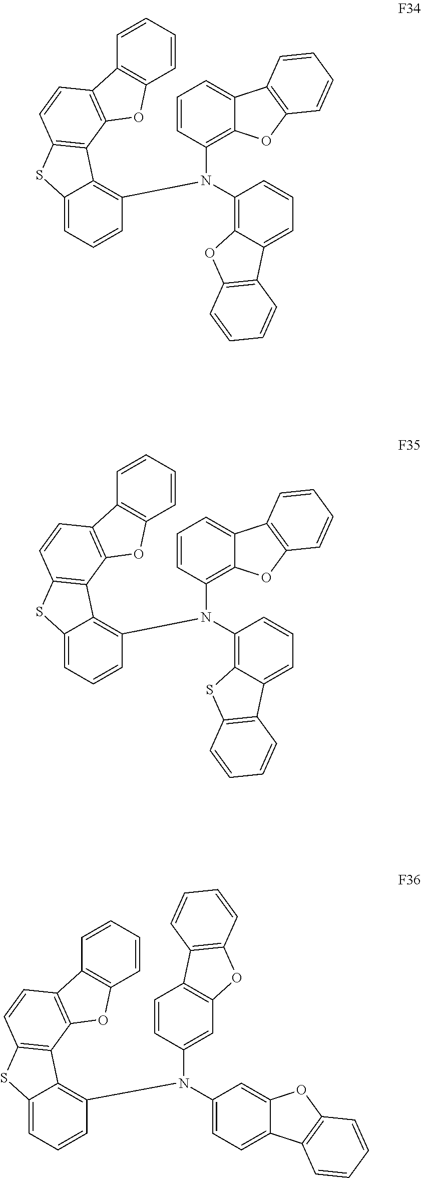 [SMILES: c1ccc2c(c1)oc1c(N(c3cccc4c3oc3ccccc34)c3cccc4sc5ccc6c7ccccc7oc6c5c34)cccc12 ; c1ccc2c(c1)oc1c(N(c3cccc4c3sc3ccccc34)c3cccc4sc5ccc6c7ccccc7oc6c5c34)cccc12 ; c1ccc2c(c1)oc1cc(N(c3ccc4c(c3)oc3ccccc34)c3cccc4sc5ccc6c7ccccc7oc6c5c34)ccc12]

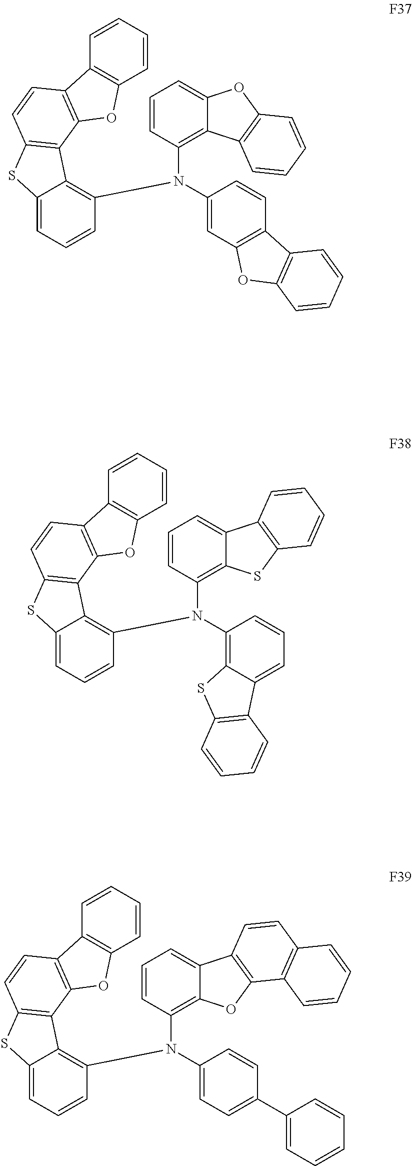 [SMILES: c1ccc2c(c1)oc1cc(N(c3cccc4oc5ccccc5c34)c3cccc4sc5ccc6c7ccccc7oc6c5c34)ccc12 ; c1ccc2c(c1)oc1c2ccc2sc3cccc(N(c4cccc5c4sc4ccccc45)c4cccc5c4sc4ccccc45)c3c21 ; c1ccc(-c2ccc(N(c3cccc4c3oc3c5ccccc5ccc43)c3cccc4sc5ccc6c7ccccc7oc6c5c34)cc2)cc1]

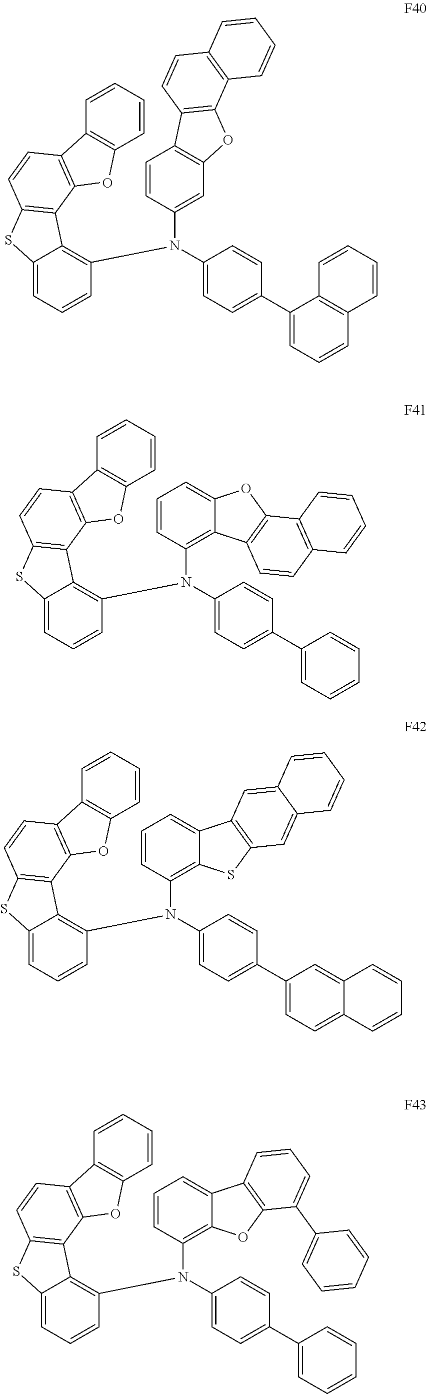 [SMILES: c1ccc2c(-c3ccc(N(c4ccc5c(c4)oc4c6ccccc6ccc54)c4cccc5sc6ccc7c8ccccc8oc7c6c45)cc3)cccc2c1 ; c1ccc(-c2ccc(N(c3cccc4oc5c6ccccc6ccc5c34)c3cccc4sc5ccc6c7ccccc7oc6c5c34)cc2)cc1 ; c1ccc2cc(-c3ccc(N(c4cccc5c4sc4cc6ccccc6cc45)c4cccc5sc6ccc7c8ccccc8oc7c6c45)cc3)ccc2c1 ; c1ccc(-c2ccc(N(c3cccc4c3oc3c(-c5ccccc5)cccc34)c3cccc4sc5ccc6c7ccccc7oc6c5c34)cc2)cc1]

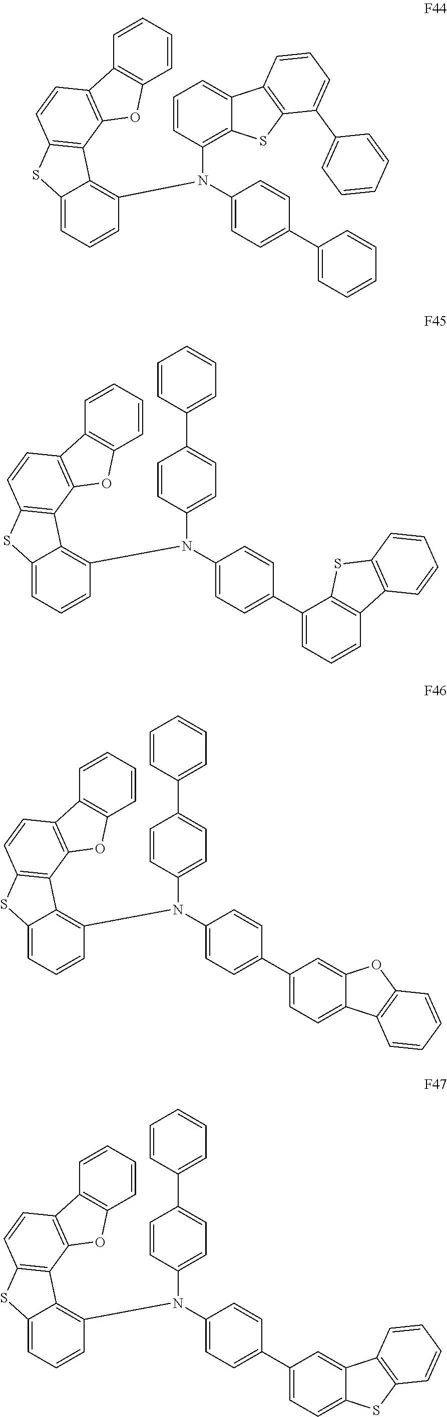 [SMILES: c1ccc(-c2ccc(N(c3cccc4c3sc3c(-c5ccccc5)cccc34)c3cccc4sc5ccc6c7ccccc7oc6c5c34)cc2)cc1 ; c1ccc(-c2ccc(N(c3ccc(-c4cccc5c4sc4ccccc45)cc3)c3cccc4sc5ccc6c7ccccc7oc6c5c34)cc2)cc1 ; c1ccc(-c2ccc(N(c3ccc(-c4ccc5c(c4)oc4ccccc45)cc3)c3cccc4sc5ccc6c7ccccc7oc6c5c34)cc2)cc1 ; c1ccc(-c2ccc(N(c3ccc(-c4ccc5sc6ccccc6c5c4)cc3)c3cccc4sc5ccc6c7ccccc7oc6c5c34)cc2)cc1]

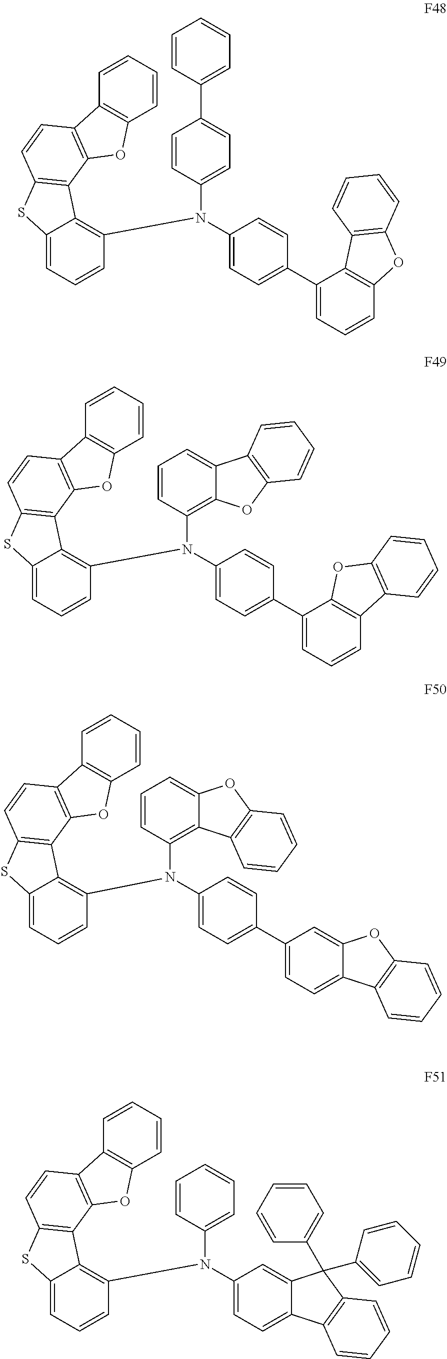 [SMILES: c1ccc(-c2ccc(N(c3ccc(-c4cccc5oc6ccccc6c45)cc3)c3cccc4sc5ccc6c7ccccc7oc6c5c34)cc2)cc1 ; c1ccc2c(c1)oc1c(-c3ccc(N(c4cccc5c4oc4ccccc45)c4cccc5sc6ccc7c8ccccc8oc7c6c45)cc3)cccc12 ; c1ccc2c(c1)oc1cc(-c3ccc(N(c4cccc5oc6ccccc6c45)c4cccc5sc6ccc7c8ccccc8oc7c6c45)cc3)ccc12 ; c1ccc(N(c2ccc3c(c2)C(c2ccccc2)(c2ccccc2)c2ccccc2-3)c2cccc3sc4ccc5c6ccccc6oc5c4c23)cc1]

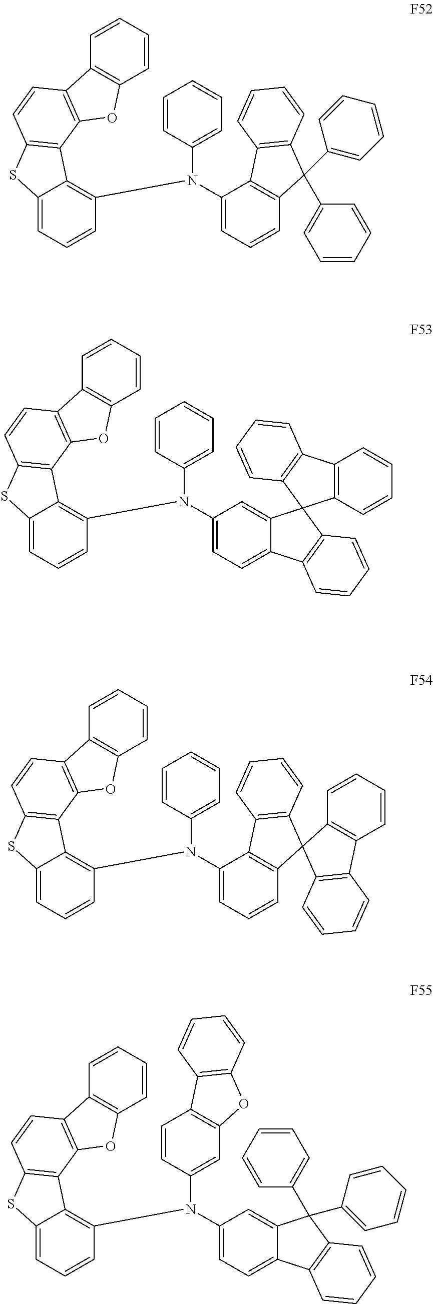 [SMILES: c1ccc(N(c2cccc3c2-c2ccccc2C3(c2ccccc2)c2ccccc2)c2cccc3sc4ccc5c6ccccc6oc5c4c23)cc1 ; c1ccc(N(c2ccc3c(c2)C2(c4ccccc4-c4ccccc42)c2ccccc2-3)c2cccc3sc4ccc5c6ccccc6oc5c4c23)cc1 ; c1ccc(N(c2cccc3c2-c2ccccc2C32c3ccccc3-c3ccccc32)c2cccc3sc4ccc5c6ccccc6oc5c4c23)cc1 ; c1ccc(C2(c3ccccc3)c3ccccc3-c3ccc(N(c4ccc5c(c4)oc4ccccc45)c4cccc5sc6ccc7c8ccccc8oc7c6c45)cc32)cc1]

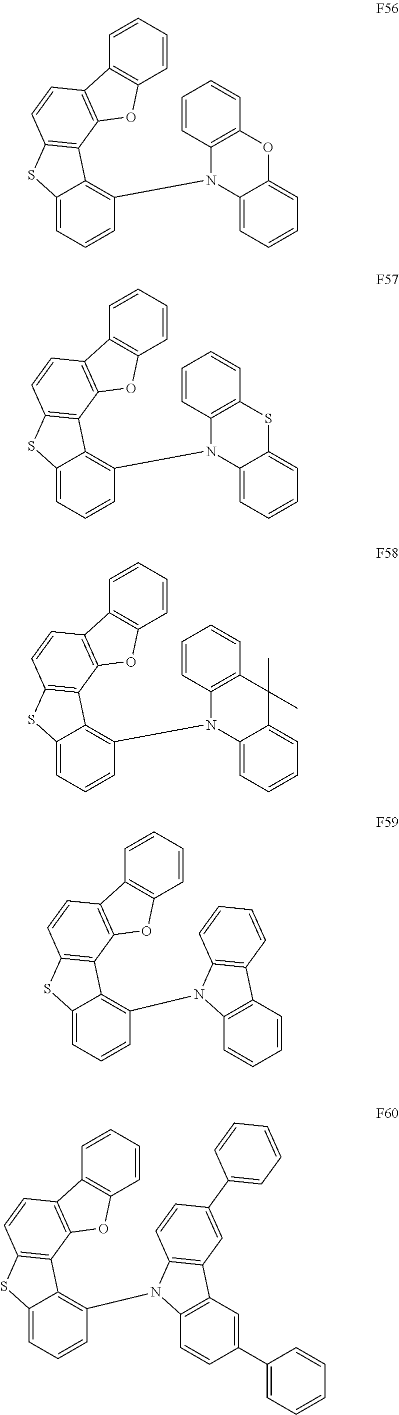 [SMILES: c1ccc2c(c1)Oc1ccccc1N2c1cccc2sc3ccc4c5ccccc5oc4c3c12 ; c1ccc2c(c1)Sc1ccccc1N2c1cccc2sc3ccc4c5ccccc5oc4c3c12 ; CC1(C)c2ccccc2N(c2cccc3sc4ccc5c6ccccc6oc5c4c23)c2ccccc21 ; c1ccc2c(c1)oc1c2ccc2sc3cccc(-n4c5ccccc5c5ccccc54)c3c21 ; c1ccc(-c2ccc3c(c2)c2cc(-c4ccccc4)ccc2n3-c2cccc3sc4ccc5c6ccccc6oc5c4c23)cc1]

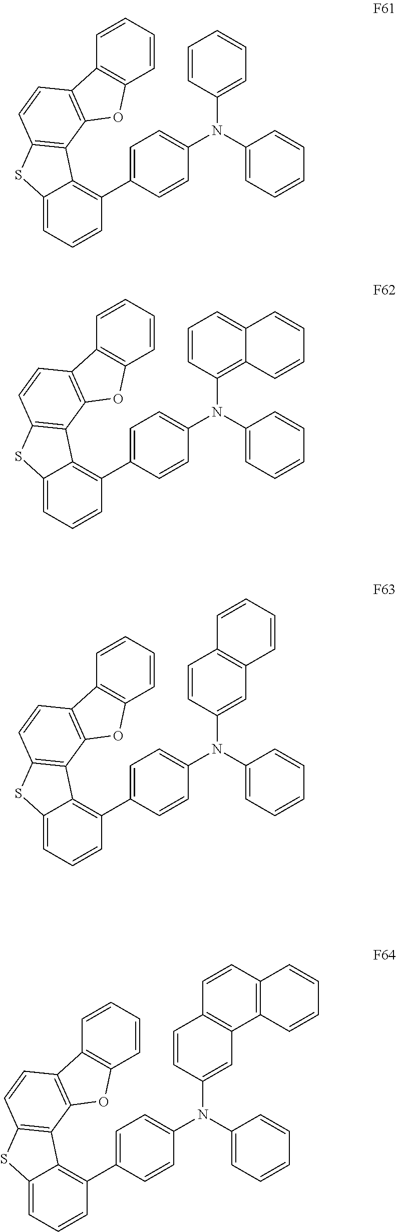 [SMILES: c1ccc(N(c2ccccc2)c2ccc(-c3cccc4sc5ccc6c7ccccc7oc6c5c34)cc2)cc1 ; c1ccc(N(c2ccc(-c3cccc4sc5ccc6c7ccccc7oc6c5c34)cc2)c2cccc3ccccc23)cc1 ; c1ccc(N(c2ccc(-c3cccc4sc5ccc6c7ccccc7oc6c5c34)cc2)c2ccc3ccccc3c2)cc1 ; c1ccc(N(c2ccc(-c3cccc4sc5ccc6c7ccccc7oc6c5c34)cc2)c2ccc3ccc4ccccc4c3c2)cc1]

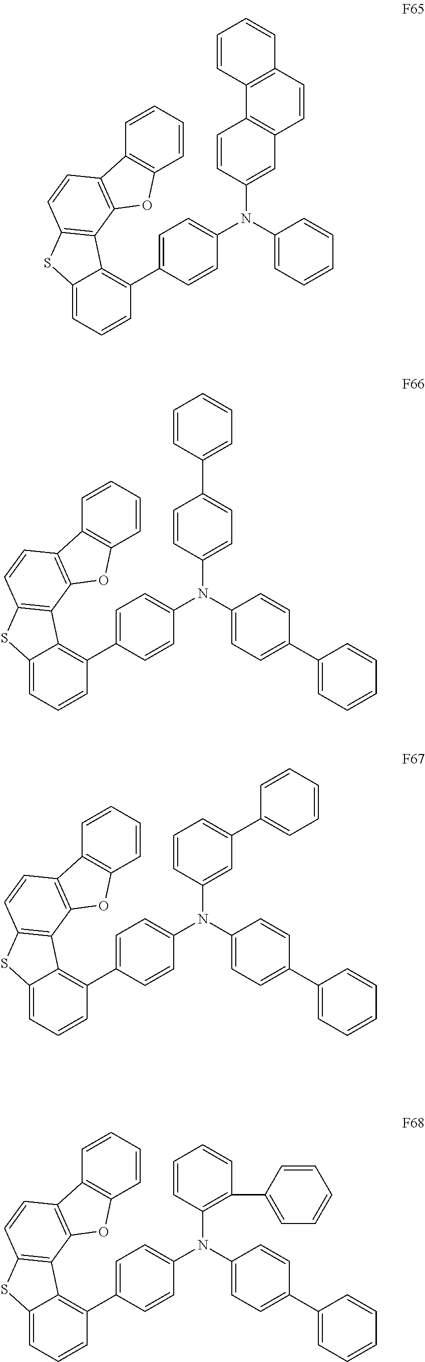 [SMILES: c1ccc(N(c2ccc(-c3cccc4sc5ccc6c7ccccc7oc6c5c34)cc2)c2ccc3c(ccc4ccccc43)c2)cc1 ; c1ccc(-c2ccc(N(c3ccc(-c4ccccc4)cc3)c3ccc(-c4cccc5sc6ccc7c8ccccc8oc7c6c45)cc3)cc2)cc1 ; c1ccc(-c2ccc(N(c3ccc(-c4cccc5sc6ccc7c8ccccc8oc7c6c45)cc3)c3cccc(-c4ccccc4)c3)cc2)cc1 ; c1ccc(-c2ccc(N(c3ccc(-c4cccc5sc6ccc7c8ccccc8oc7c6c45)cc3)c3ccccc3-c3ccccc3)cc2)cc1]

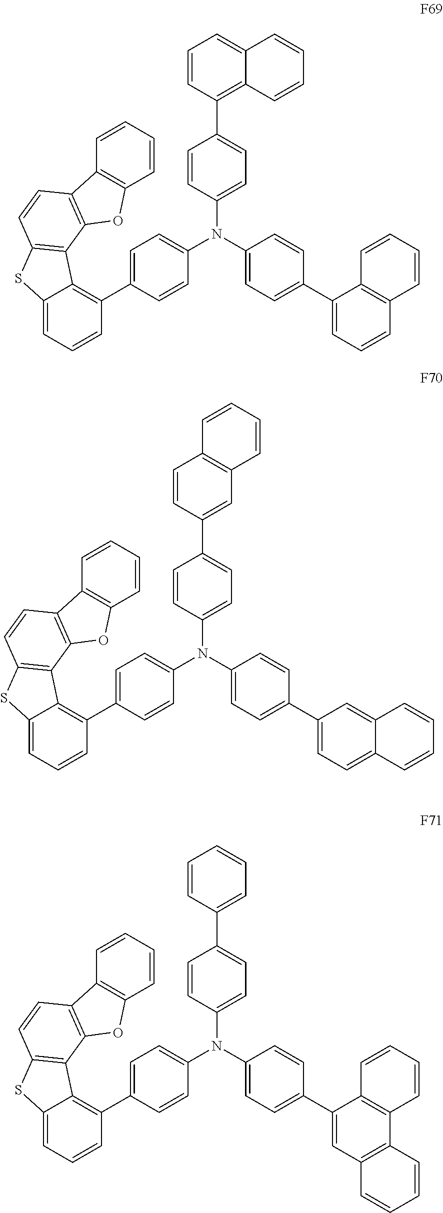 [SMILES: c1ccc2c(-c3ccc(N(c4ccc(-c5cccc6ccccc56)cc4)c4ccc(-c5cccc6sc7ccc8c9ccccc9oc8c7c56)cc4)cc3)cccc2c1 ; c1ccc2cc(-c3ccc(N(c4ccc(-c5ccc6ccccc6c5)cc4)c4ccc(-c5cccc6sc7ccc8c9ccccc9oc8c7c56)cc4)cc3)ccc2c1 ; c1ccc(-c2ccc(N(c3ccc(-c4cc5ccccc5c5ccccc45)cc3)c3ccc(-c4cccc5sc6ccc7c8ccccc8oc7c6c45)cc3)cc2)cc1]

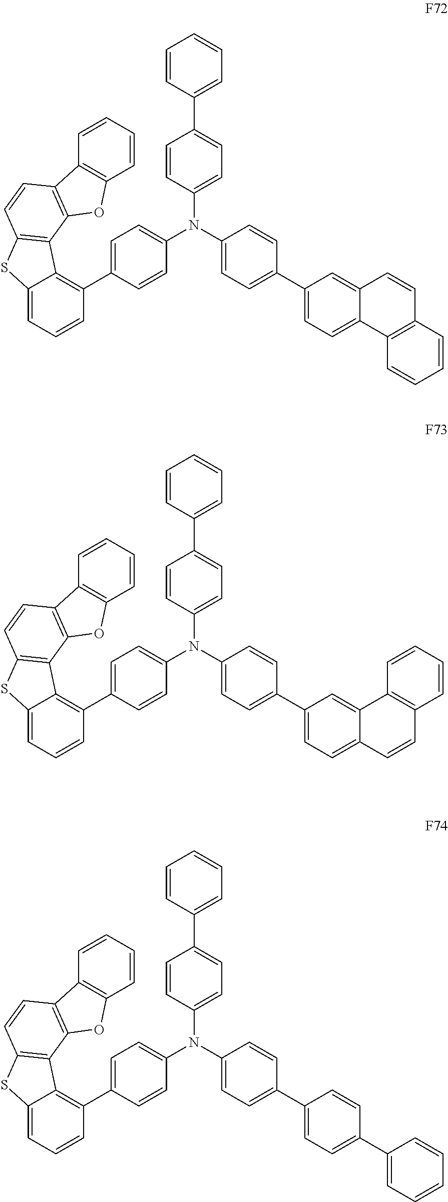 [SMILES: c1ccc(-c2ccc(N(c3ccc(-c4ccc5c(ccc6ccccc65)c4)cc3)c3ccc(-c4cccc5sc6ccc7c8ccccc8oc7c6c45)cc3)cc2)cc1 ; c1ccc(-c2ccc(N(c3ccc(-c4ccc5ccc6ccccc6c5c4)cc3)c3ccc(-c4cccc5sc6ccc7c8ccccc8oc7c6c45)cc3)cc2)cc1 ; c1ccc(-c2ccc(-c3ccc(N(c4ccc(-c5ccccc5)cc4)c4ccc(-c5cccc6sc7ccc8c9ccccc9oc8c7c56)cc4)cc3)cc2)cc1]

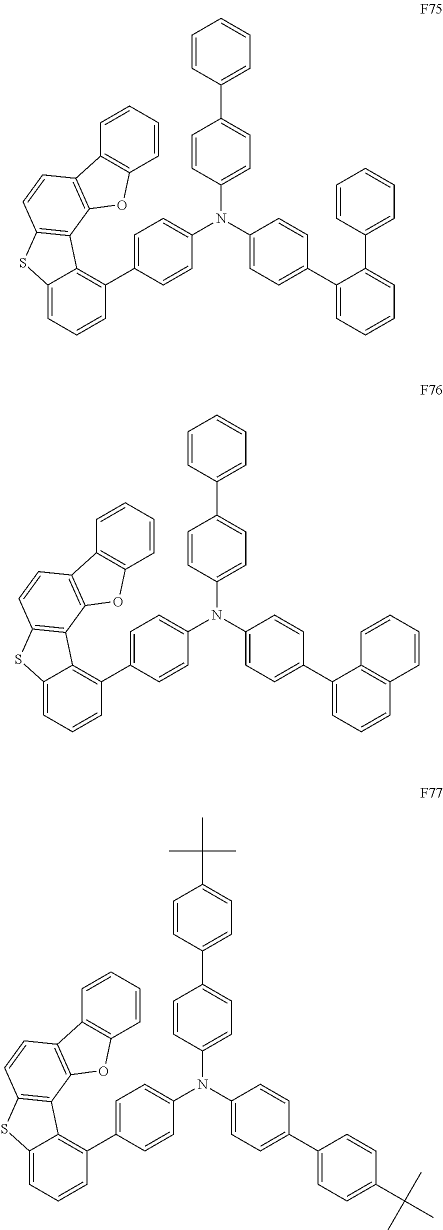 [SMILES: c1ccc(-c2ccc(N(c3ccc(-c4ccccc4-c4ccccc4)cc3)c3ccc(-c4cccc5sc6ccc7c8ccccc8oc7c6c45)cc3)cc2)cc1 ; c1ccc(-c2ccc(N(c3ccc(-c4cccc5ccccc45)cc3)c3ccc(-c4cccc5sc6ccc7c8ccccc8oc7c6c45)cc3)cc2)cc1 ; CC(C)(C)c1ccc(-c2ccc(N(c3ccc(-c4ccc(C(C)(C)C)cc4)cc3)c3ccc(-c4cccc5sc6ccc7c8ccccc8oc7c6c45)cc3)cc2)cc1]

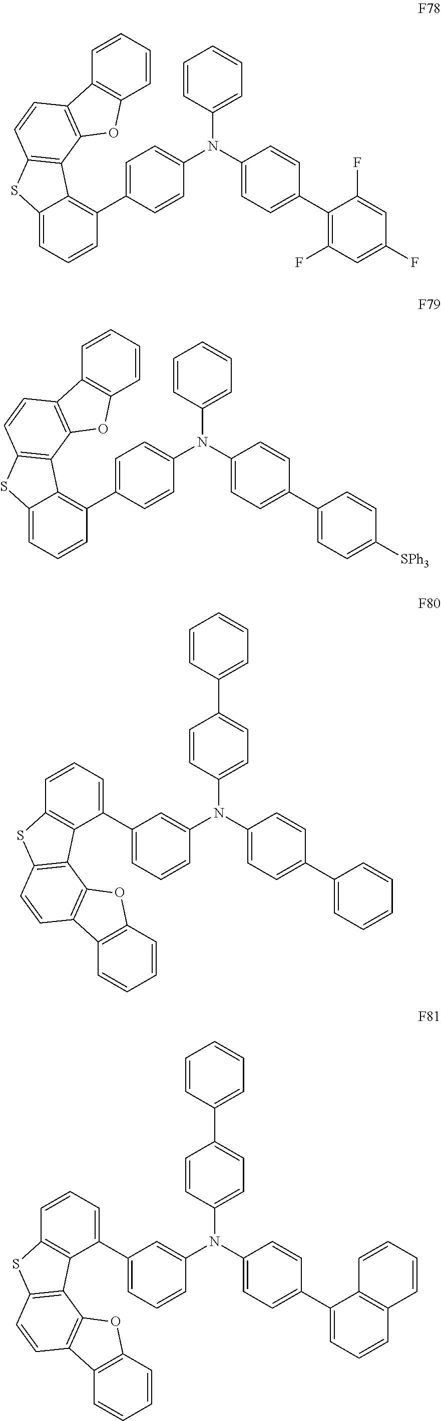

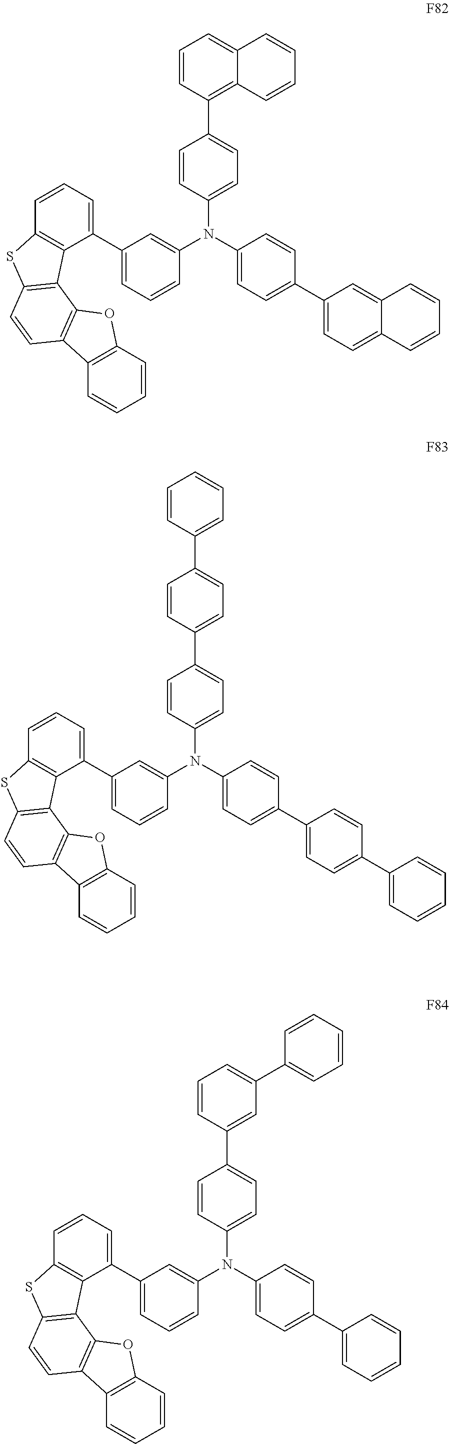 [SMILES: c1cc(-c2cccc3sc4ccc5c6ccccc6oc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6sc7ccc8c9ccccc9oc8c7c56)c4)cc3)cc2)cc1 ; c1ccc(-c2ccc(N(c3ccc(-c4cccc(-c5ccccc5)c4)cc3)c3cccc(-c4cccc5sc6ccc7c8ccccc8oc7c6c45)c3)cc2)cc1]

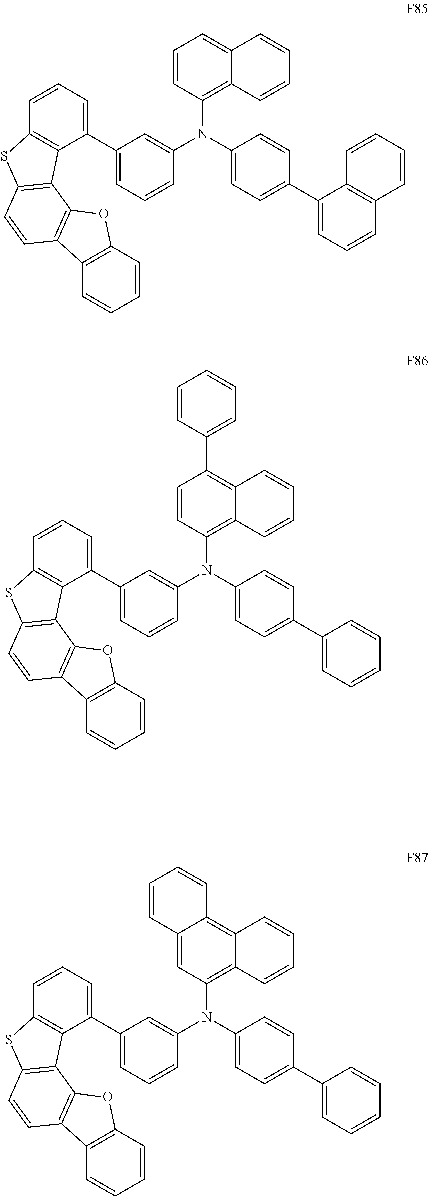 [SMILES: c1cc(-c2cccc3sc4ccc5c6ccccc6oc5c4c23)cc(N(c2ccc(-c3cccc4ccccc34)cc2)c2cccc3ccccc23)c1 ; c1ccc(-c2ccc(N(c3cccc(-c4cccc5sc6ccc7c8ccccc8oc7c6c45)c3)c3ccc(-c4ccccc4)c4ccccc34)cc2)cc1 ; c1ccc(-c2ccc(N(c3cccc(-c4cccc5sc6ccc7c8ccccc8oc7c6c45)c3)c3cc4ccccc4c4ccccc34)cc2)cc1]

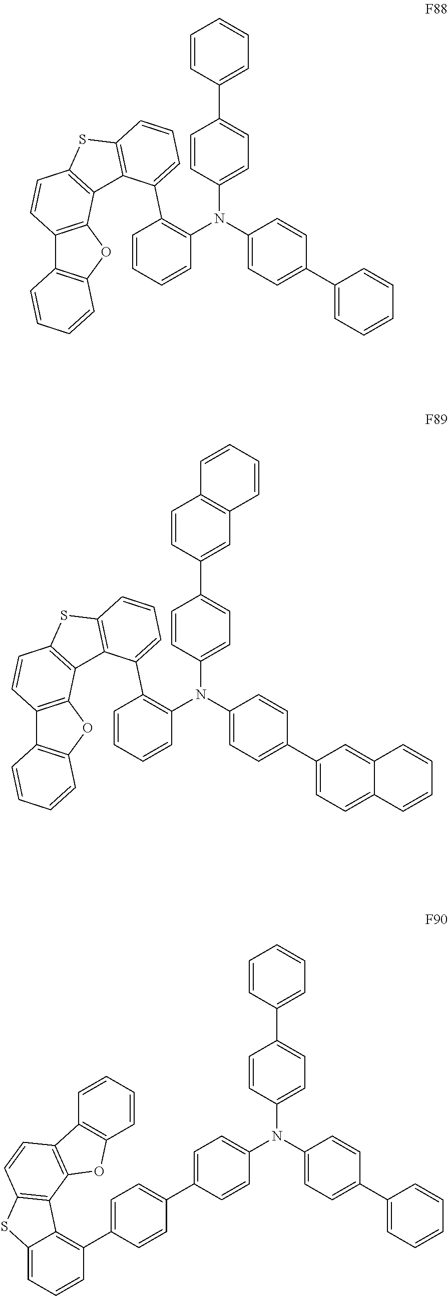 [SMILES: c1ccc(-c2ccc(N(c3ccc(-c4ccccc4)cc3)c3ccccc3-c3cccc4sc5ccc6c7ccccc7oc6c5c34)cc2)cc1 ; c1ccc(N(c2ccc(-c3ccc4ccccc4c3)cc2)c2ccc(-c3ccc4ccccc4c3)cc2)c(-c2cccc3sc4ccc5c6ccccc6oc5c4c23)c1 ; c1ccc(-c2ccc(N(c3ccc(-c4ccccc4)cc3)c3ccc(-c4ccc(-c5cccc6sc7ccc8c9ccccc9oc8c7c56)cc4)cc3)cc2)cc1]

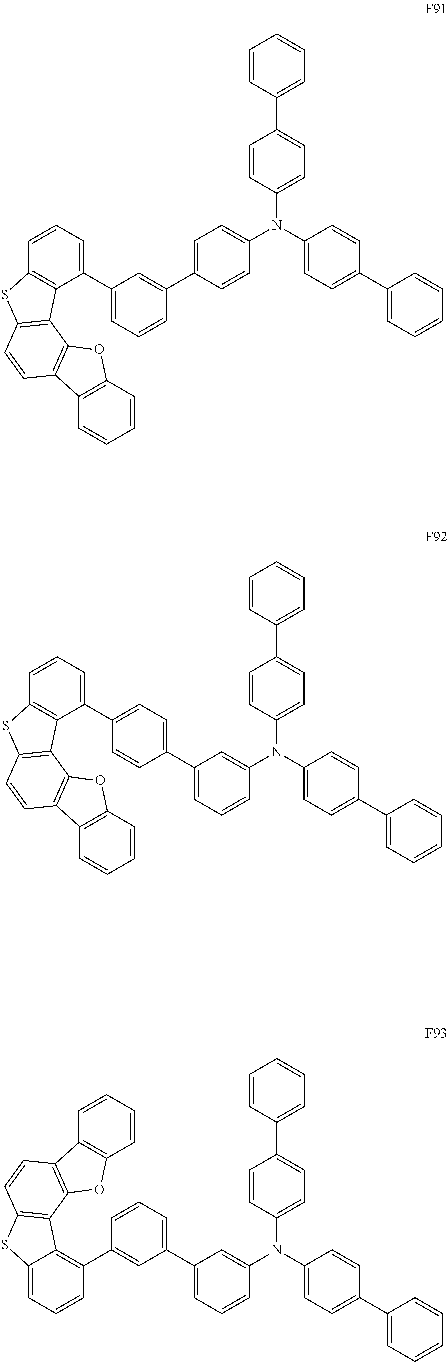 [SMILES: c1ccc(-c2ccc(N(c3ccc(-c4ccccc4)cc3)c3ccc(-c4cccc(-c5cccc6sc7ccc8c9ccccc9oc8c7c56)c4)cc3)cc2)cc1 ; c1ccc(-c2ccc(N(c3ccc(-c4ccccc4)cc3)c3cccc(-c4ccc(-c5cccc6sc7ccc8c9ccccc9oc8c7c56)cc4)c3)cc2)cc1 ; c1ccc(-c2ccc(N(c3ccc(-c4ccccc4)cc3)c3cccc(-c4cccc(-c5cccc6sc7ccc8c9ccccc9oc8c7c56)c4)c3)cc2)cc1]

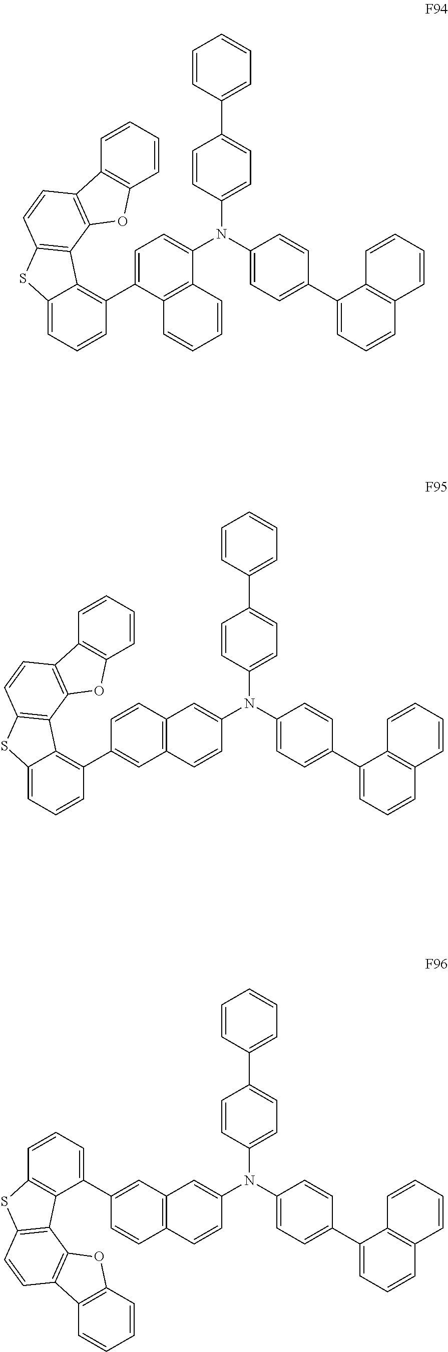 [SMILES: c1ccc(-c2ccc(N(c3ccc(-c4cccc5ccccc45)cc3)c3ccc(-c4cccc5sc6ccc7c8ccccc8oc7c6c45)c4ccccc34)cc2)cc1 ; c1ccc(-c2ccc(N(c3ccc(-c4cccc5ccccc45)cc3)c3ccc4cc(-c5cccc6sc7ccc8c9ccccc9oc8c7c56)ccc4c3)cc2)cc1 ; c1ccc(-c2ccc(N(c3ccc(-c4cccc5ccccc45)cc3)c3ccc4ccc(-c5cccc6sc7ccc8c9ccccc9oc8c7c56)cc4c3)cc2)cc1]

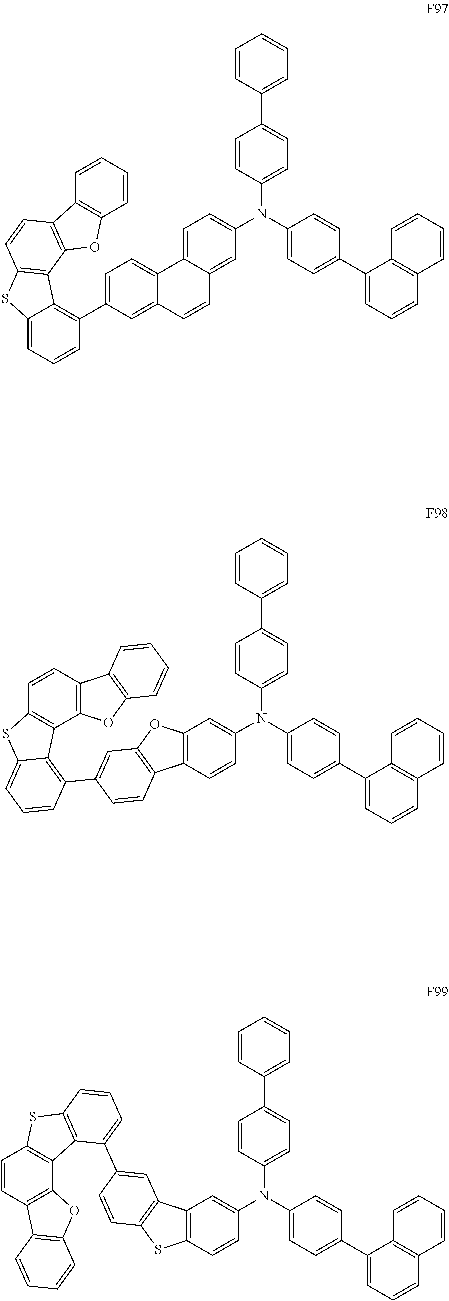 [SMILES: c1ccc(-c2ccc(N(c3ccc(-c4cccc5ccccc45)cc3)c3ccc4c(ccc5cc(-c6cccc7sc8ccc9c%10ccccc%10oc9c8c67)ccc54)c3)cc2)cc1 ; c1ccc(-c2ccc(N(c3ccc(-c4cccc5ccccc45)cc3)c3ccc4c(c3)oc3cc(-c5cccc6sc7ccc8c9ccccc9oc8c7c56)ccc34)cc2)cc1 ; c1ccc(-c2ccc(N(c3ccc(-c4cccc5ccccc45)cc3)c3ccc4sc5ccc(-c6cccc7sc8ccc9c%10ccccc%10oc9c8c67)cc5c4c3)cc2)cc1]

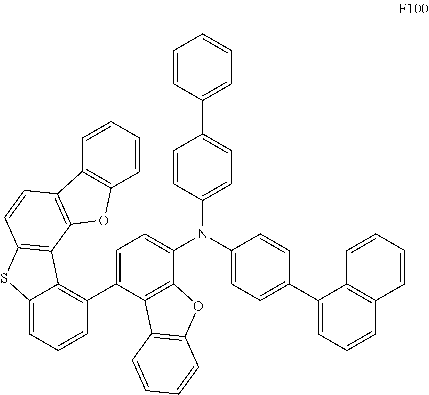 [SMILES: c1ccc(-c2ccc(N(c3ccc(-c4cccc5ccccc45)cc3)c3ccc(-c4cccc5sc6ccc7c8ccccc8oc7c6c45)c4c3oc3ccccc34)cc2)cc1]

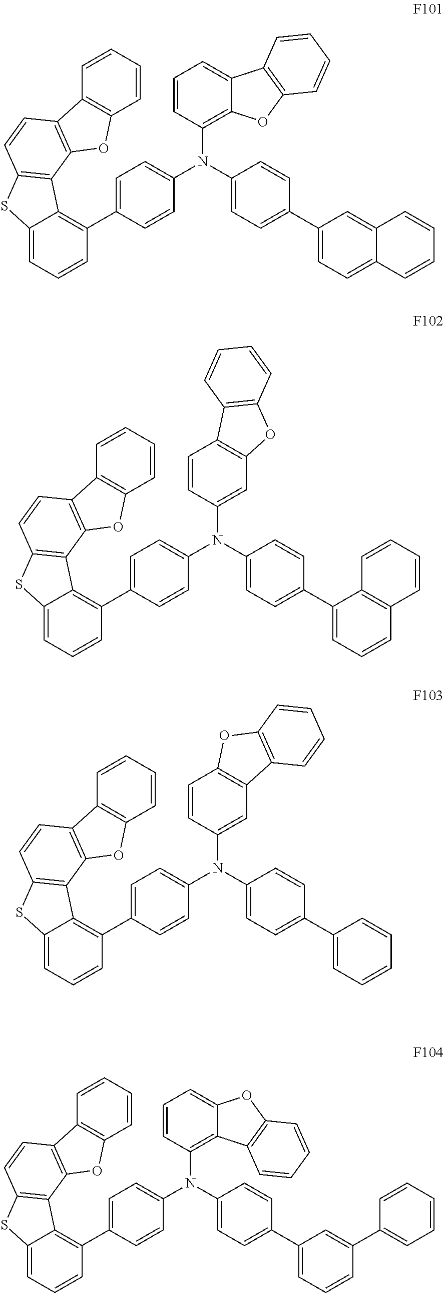 [SMILES: c1ccc2cc(-c3ccc(N(c4ccc(-c5cccc6sc7ccc8c9ccccc9oc8c7c56)cc4)c4cccc5c4oc4ccccc45)cc3)ccc2c1 ; c1ccc2c(-c3ccc(N(c4ccc(-c5cccc6sc7ccc8c9ccccc9oc8c7c56)cc4)c4ccc5c(c4)oc4ccccc45)cc3)cccc2c1 ; c1ccc(-c2ccc(N(c3ccc(-c4cccc5sc6ccc7c8ccccc8oc7c6c45)cc3)c3ccc4oc5ccccc5c4c3)cc2)cc1 ; c1ccc(-c2cccc(-c3ccc(N(c4ccc(-c5cccc6sc7ccc8c9ccccc9oc8c7c56)cc4)c4cccc5oc6ccccc6c45)cc3)c2)cc1]

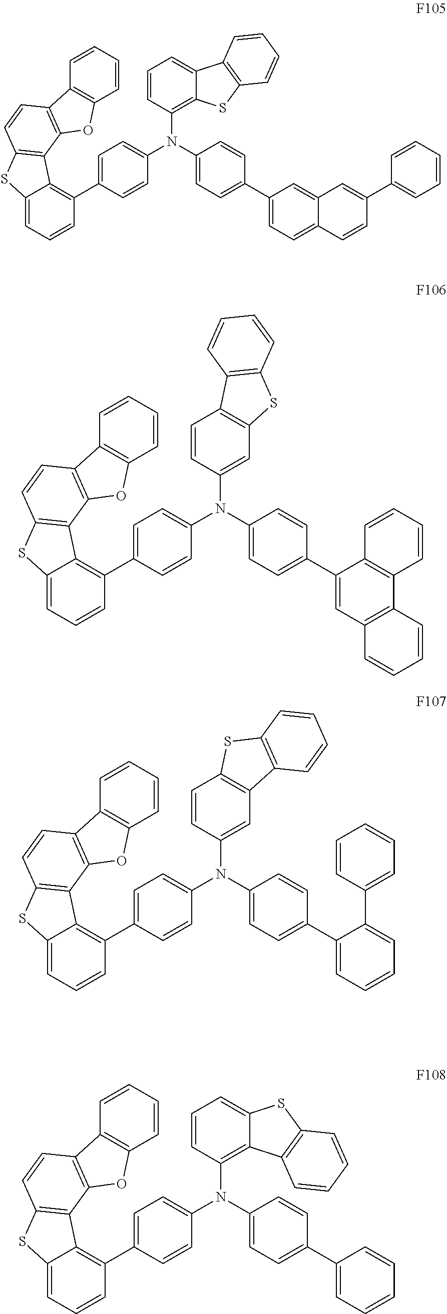 [SMILES: c1ccc(-c2ccc3ccc(-c4ccc(N(c5ccc(-c6cccc7sc8ccc9c%10ccccc%10oc9c8c67)cc5)c5cccc6c5sc5ccccc56)cc4)cc3c2)cc1 ; c1ccc2c(c1)cc(-c1ccc(N(c3ccc(-c4cccc5sc6ccc7c8ccccc8oc7c6c45)cc3)c3ccc4c(c3)sc3ccccc34)cc1)c1ccccc12 ; c1ccc(-c2ccccc2-c2ccc(N(c3ccc(-c4cccc5sc6ccc7c8ccccc8oc7c6c45)cc3)c3ccc4sc5ccccc5c4c3)cc2)cc1 ; c1ccc(-c2ccc(N(c3ccc(-c4cccc5sc6ccc7c8ccccc8oc7c6c45)cc3)c3cccc4sc5ccccc5c34)cc2)cc1]

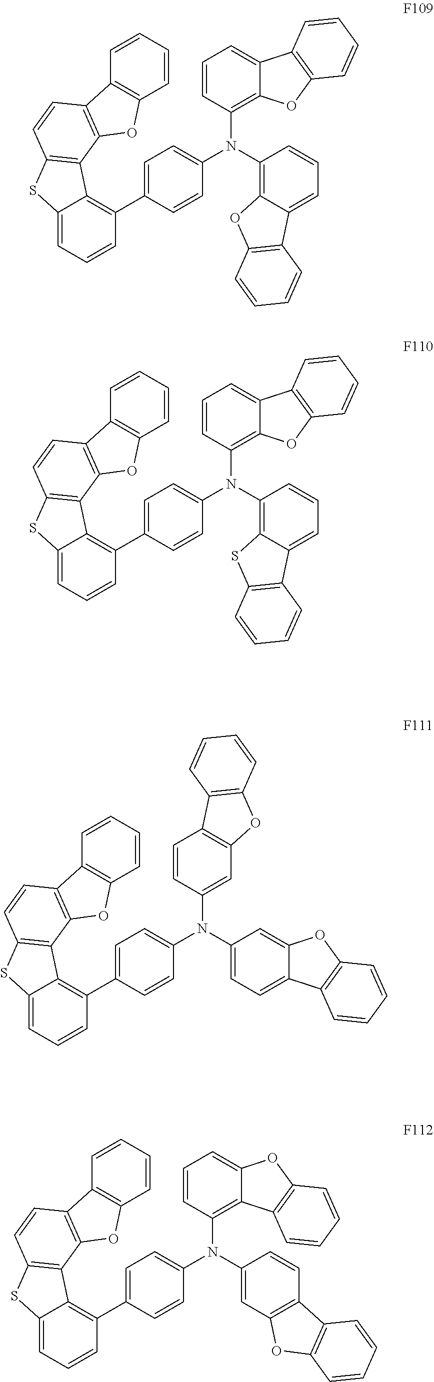 [SMILES: c1ccc2c(c1)oc1c(N(c3ccc(-c4cccc5sc6ccc7c8ccccc8oc7c6c45)cc3)c3cccc4c3oc3ccccc34)cccc12 ; c1ccc2c(c1)oc1c(N(c3ccc(-c4cccc5sc6ccc7c8ccccc8oc7c6c45)cc3)c3cccc4c3sc3ccccc34)cccc12 ; c1ccc2c(c1)oc1cc(N(c3ccc(-c4cccc5sc6ccc7c8ccccc8oc7c6c45)cc3)c3ccc4c(c3)oc3ccccc34)ccc12 ; c1ccc2c(c1)oc1cc(N(c3ccc(-c4cccc5sc6ccc7c8ccccc8oc7c6c45)cc3)c3cccc4oc5ccccc5c34)ccc12]

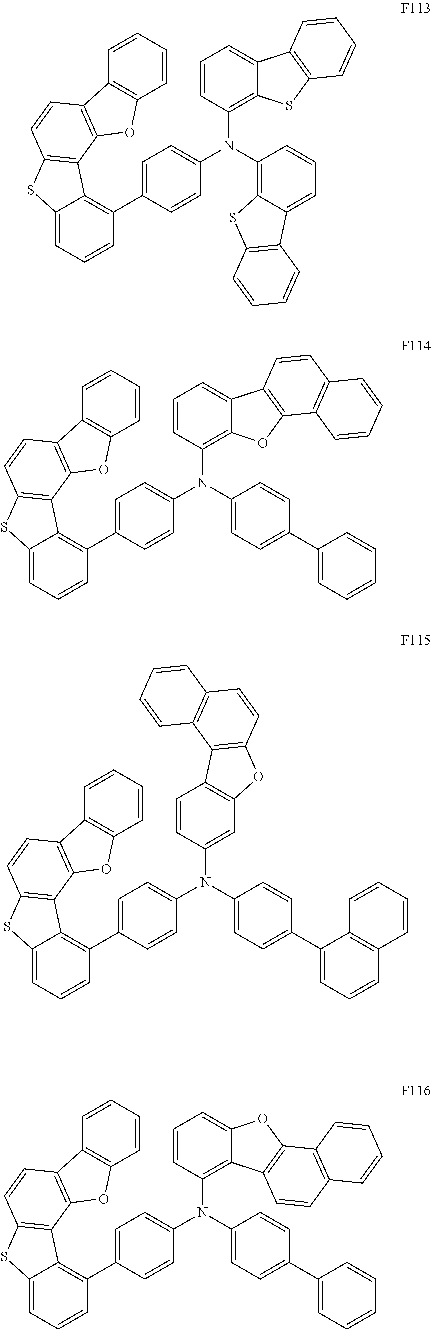 [SMILES: c1ccc2c(c1)oc1c2ccc2sc3cccc(-c4ccc(N(c5cccc6c5sc5ccccc56)c5cccc6c5sc5ccccc56)cc4)c3c21 ; c1ccc(-c2ccc(N(c3ccc(-c4cccc5sc6ccc7c8ccccc8oc7c6c45)cc3)c3cccc4c3oc3c5ccccc5ccc43)cc2)cc1 ; c1ccc2c(-c3ccc(N(c4ccc(-c5cccc6sc7ccc8c9ccccc9oc8c7c56)cc4)c4ccc5c(c4)oc4ccc6ccccc6c45)cc3)cccc2c1 ; c1ccc(-c2ccc(N(c3ccc(-c4cccc5sc6ccc7c8ccccc8oc7c6c45)cc3)c3cccc4oc5c6ccccc6ccc5c34)cc2)cc1]

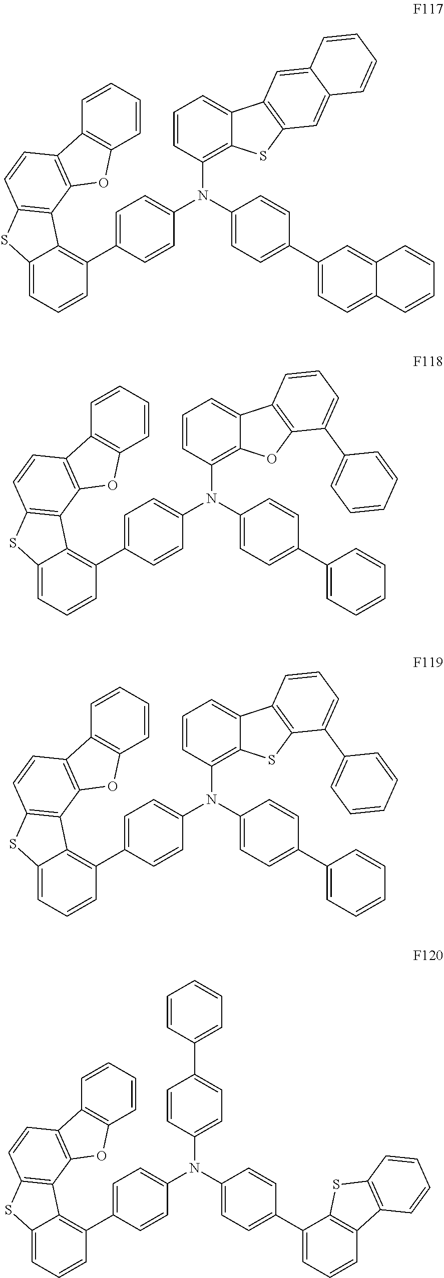 [SMILES: c1ccc2cc(-c3ccc(N(c4ccc(-c5cccc6sc7ccc8c9ccccc9oc8c7c56)cc4)c4cccc5c4sc4cc6ccccc6cc45)cc3)ccc2c1 ; c1ccc(-c2ccc(N(c3ccc(-c4cccc5sc6ccc7c8ccccc8oc7c6c45)cc3)c3cccc4c3oc3c(-c5ccccc5)cccc34)cc2)cc1 ; c1ccc(-c2ccc(N(c3ccc(-c4cccc5sc6ccc7c8ccccc8oc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5sc6ccc7c8ccccc8oc7c6c45)cc3)cc2)cc1]

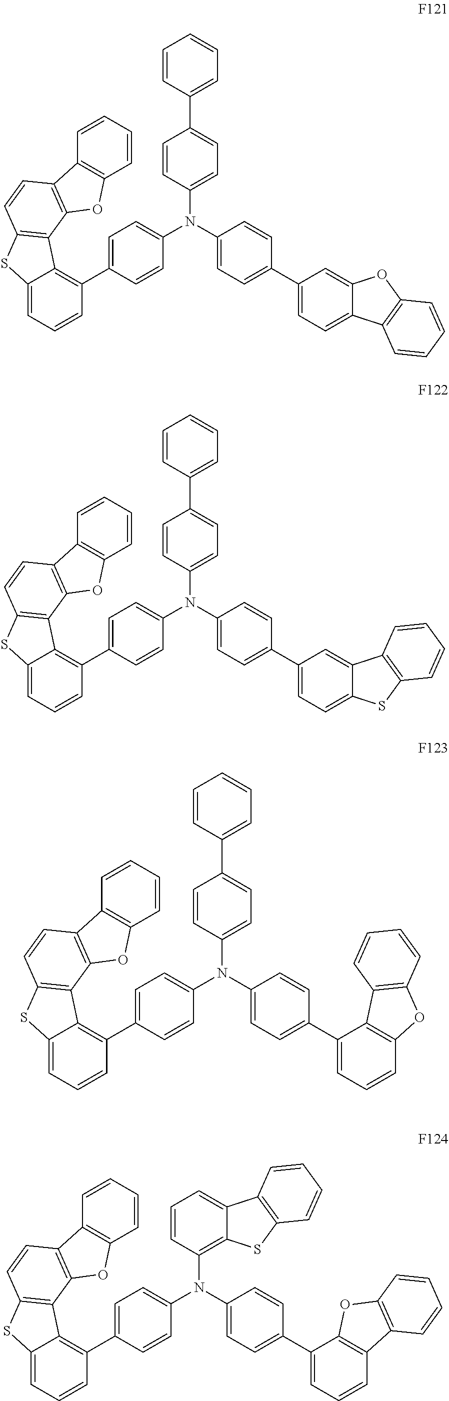 [SMILES: c1ccc(-c2ccc(N(c3ccc(-c4ccc5c(c4)oc4ccccc45)cc3)c3ccc(-c4cccc5sc6ccc7c8ccccc8oc7c6c45)cc3)cc2)cc1 ; c1ccc(-c2ccc(N(c3ccc(-c4ccc5sc6ccccc6c5c4)cc3)c3ccc(-c4cccc5sc6ccc7c8ccccc8oc7c6c45)cc3)cc2)cc1 ; c1ccc(-c2ccc(N(c3ccc(-c4cccc5oc6ccccc6c45)cc3)c3ccc(-c4cccc5sc6ccc7c8ccccc8oc7c6c45)cc3)cc2)cc1 ; c1ccc2c(c1)oc1c(-c3ccc(N(c4ccc(-c5cccc6sc7ccc8c9ccccc9oc8c7c56)cc4)c4cccc5c4sc4ccccc45)cc3)cccc12]

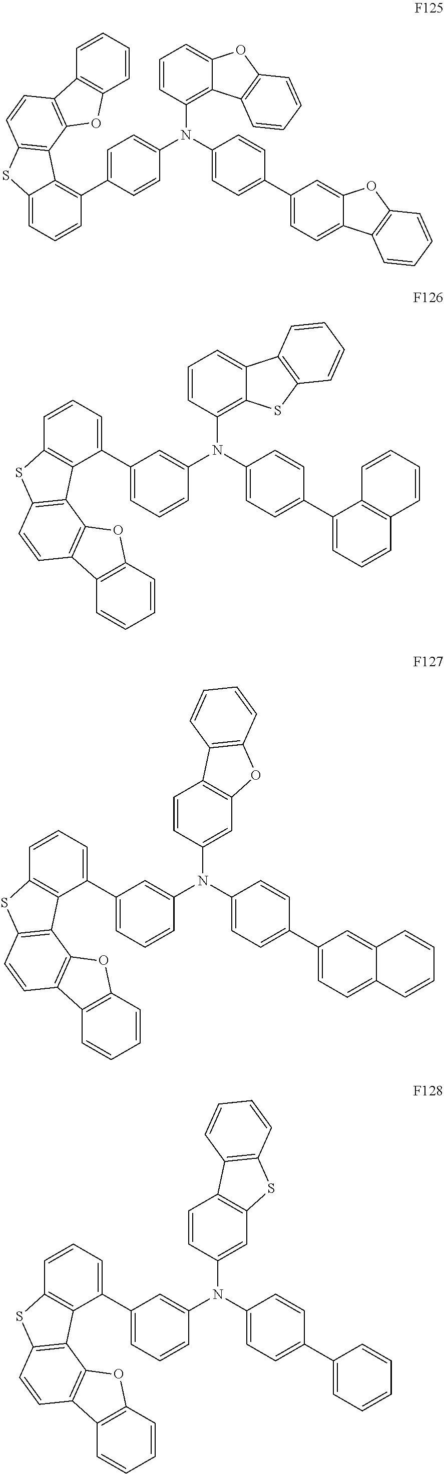 [SMILES: c1ccc2c(c1)oc1cc(-c3ccc(N(c4ccc(-c5cccc6sc7ccc8c9ccccc9oc8c7c56)cc4)c4cccc5oc6ccccc6c45)cc3)ccc12 ; c1cc(-c2cccc3sc4ccc5c6ccccc6oc5c4c23)cc(N(c2ccc(-c3cccc4ccccc34)cc2)c2cccc3c2sc2ccccc23)c1 ; c1cc(-c2cccc3sc4ccc5c6ccccc6oc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5sc6ccc7c8ccccc8oc7c6c45)c3)c3ccc4c(c3)sc3ccccc34)cc2)cc1]

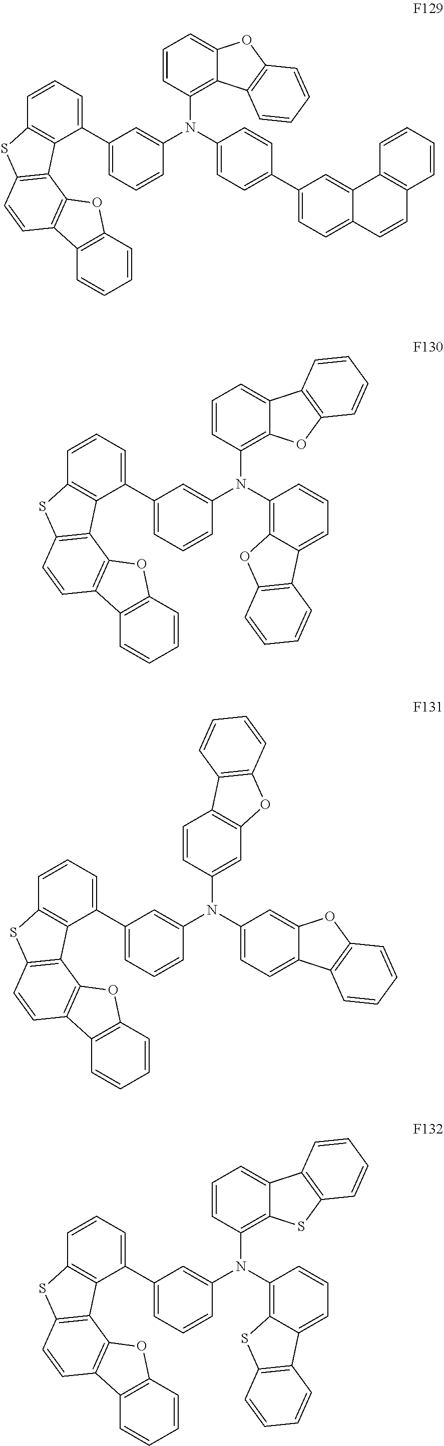

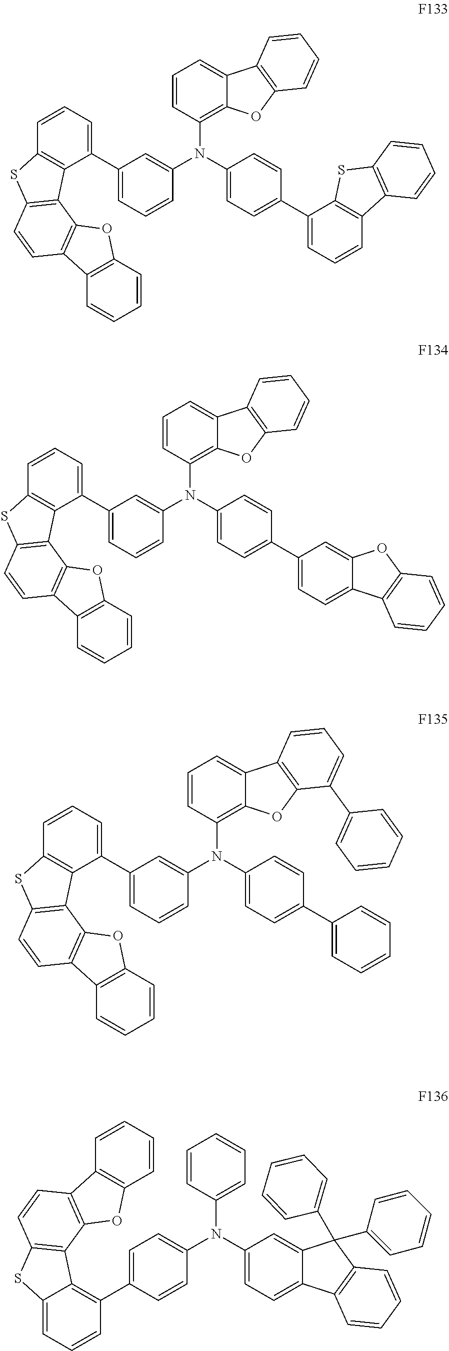 [SMILES: c1cc(-c2cccc3sc4ccc5c6ccccc6oc5c4c23)cc(N(c2ccc(-c3cccc4c3sc3ccccc34)cc2)c2cccc3c2oc2ccccc23)c1 ; c1cc(-c2cccc3sc4ccc5c6ccccc6oc5c4c23)cc(N(c2ccc(-c3ccc4c(c3)oc3ccccc34)cc2)c2cccc3c2oc2ccccc23)c1 ; c1ccc(-c2ccc(N(c3cccc(-c4cccc5sc6ccc7c8ccccc8oc7c6c45)c3)c3cccc4c3oc3c(-c5ccccc5)cccc34)cc2)cc1 ; c1ccc(N(c2ccc(-c3cccc4sc5ccc6c7ccccc7oc6c5c34)cc2)c2ccc3c(c2)C(c2ccccc2)(c2ccccc2)c2ccccc2-3)cc1]

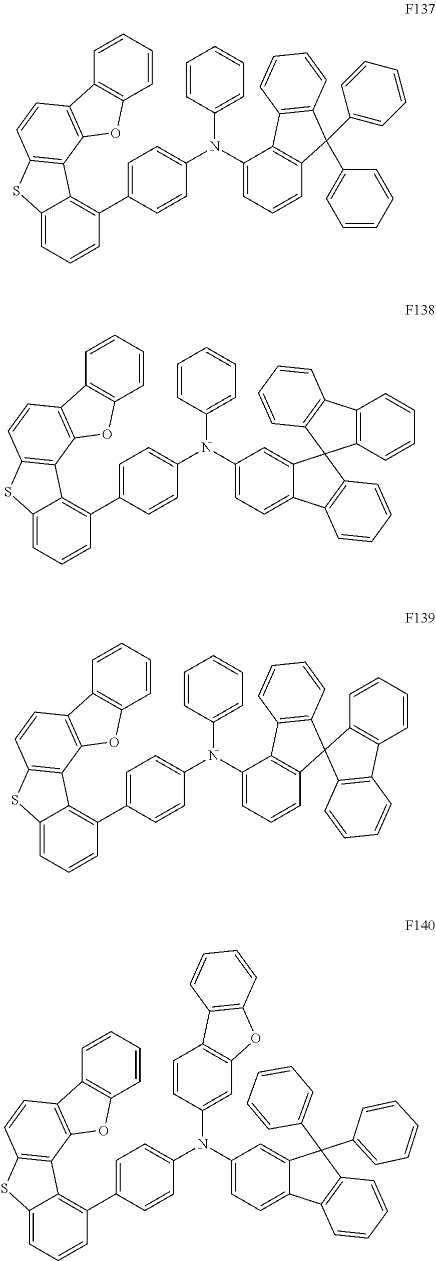 [SMILES: c1ccc(N(c2ccc(-c3cccc4sc5ccc6c7ccccc7oc6c5c34)cc2)c2cccc3c2-c2ccccc2C3(c2ccccc2)c2ccccc2)cc1 ; c1ccc(N(c2ccc(-c3cccc4sc5ccc6c7ccccc7oc6c5c34)cc2)c2ccc3c(c2)C2(c4ccccc4-c4ccccc42)c2ccccc2-3)cc1 ; c1ccc(N(c2ccc(-c3cccc4sc5ccc6c7ccccc7oc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6sc7ccc8c9ccccc9oc8c7c56)cc4)c4ccc5c(c4)oc4ccccc45)cc32)cc1]

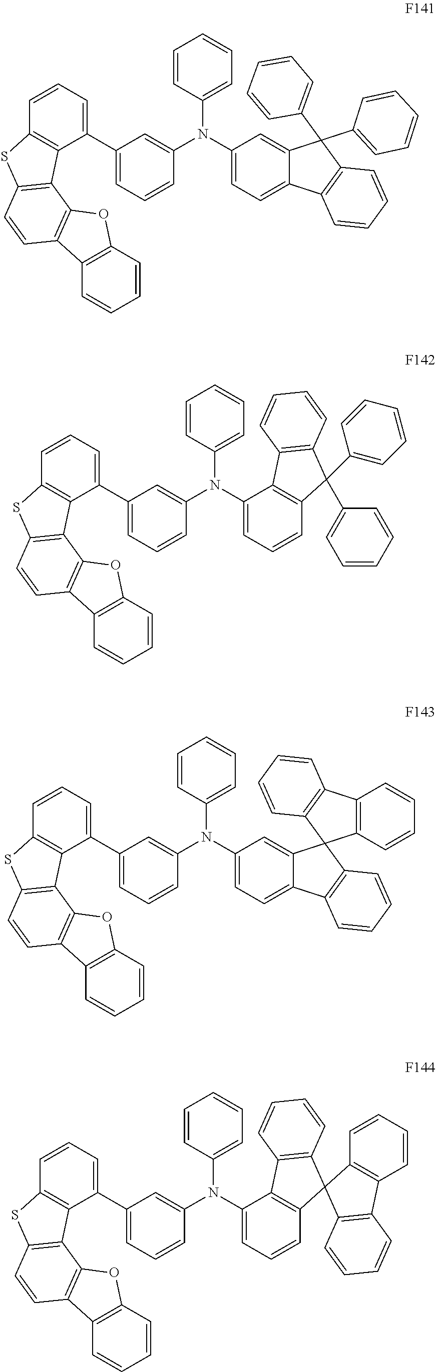 [SMILES: c1ccc(N(c2cccc(-c3cccc4sc5ccc6c7ccccc7oc6c5c34)c2)c2ccc3c(c2)C(c2ccccc2)(c2ccccc2)c2ccccc2-3)cc1 ; c1ccc(N(c2cccc(-c3cccc4sc5ccc6c7ccccc7oc6c5c34)c2)c2cccc3c2-c2ccccc2C3(c2ccccc2)c2ccccc2)cc1 ; c1ccc(N(c2cccc(-c3cccc4sc5ccc6c7ccccc7oc6c5c34)c2)c2ccc3c(c2)C2(c4ccccc4-c4ccccc42)c2ccccc2-3)cc1 ; c1ccc(N(c2cccc(-c3cccc4sc5ccc6c7ccccc7oc6c5c34)c2)c2cccc3c2-c2ccccc2C32c3ccccc3-c3ccccc32)cc1]

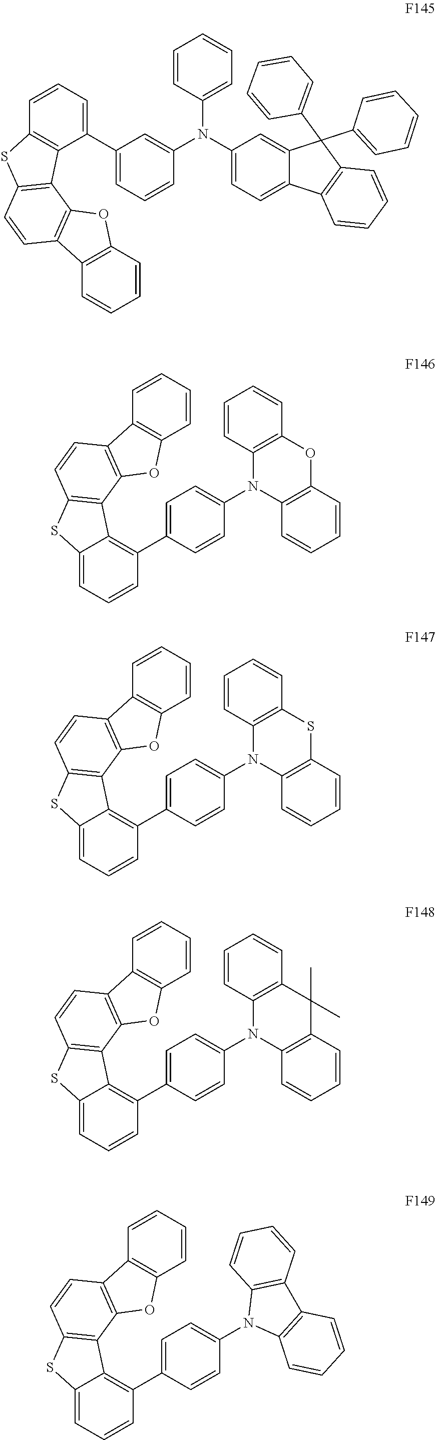 [SMILES: c1ccc(N(c2cccc(-c3cccc4sc5ccc6c7ccccc7oc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3sc4ccc5c6ccccc6oc5c4c23)cc1 ; c1ccc2c(c1)Sc1ccccc1N2c1ccc(-c2cccc3sc4ccc5c6ccccc6oc5c4c23)cc1 ; CC1(C)c2ccccc2N(c2ccc(-c3cccc4sc5ccc6c7ccccc7oc6c5c34)cc2)c2ccccc21 ; c1ccc2c(c1)oc1c2ccc2sc3cccc(-c4ccc(-n5c6ccccc6c6ccccc65)cc4)c3c21]

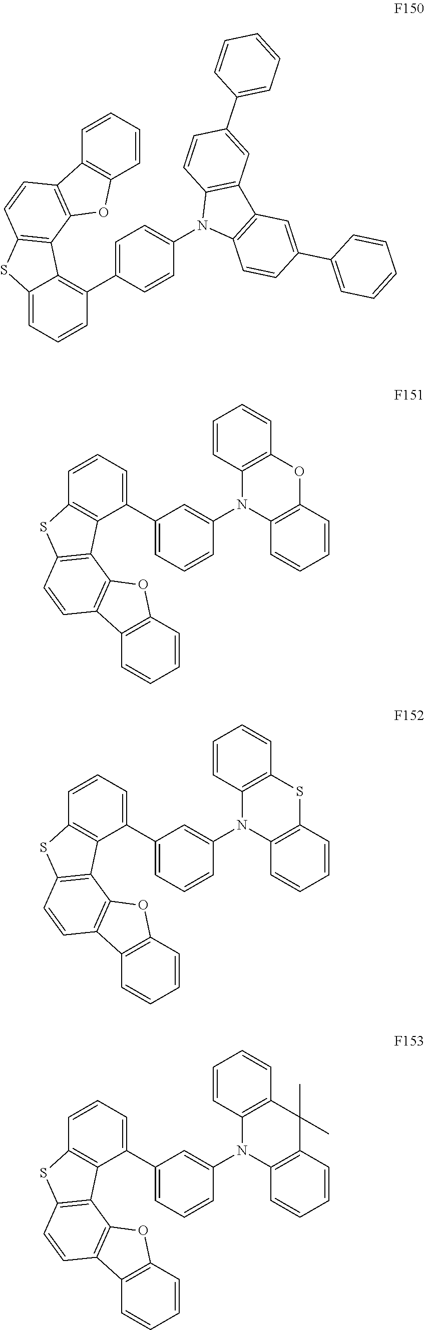 [SMILES: c1ccc(-c2ccc3c(c2)c2cc(-c4ccccc4)ccc2n3-c2ccc(-c3cccc4sc5ccc6c7ccccc7oc6c5c34)cc2)cc1 ; c1cc(-c2cccc3sc4ccc5c6ccccc6oc5c4c23)cc(N2c3ccccc3Oc3ccccc32)c1 ; c1cc(-c2cccc3sc4ccc5c6ccccc6oc5c4c23)cc(N2c3ccccc3Sc3ccccc32)c1 ; CC1(C)c2ccccc2N(c2cccc(-c3cccc4sc5ccc6c7ccccc7oc6c5c34)c2)c2ccccc21]

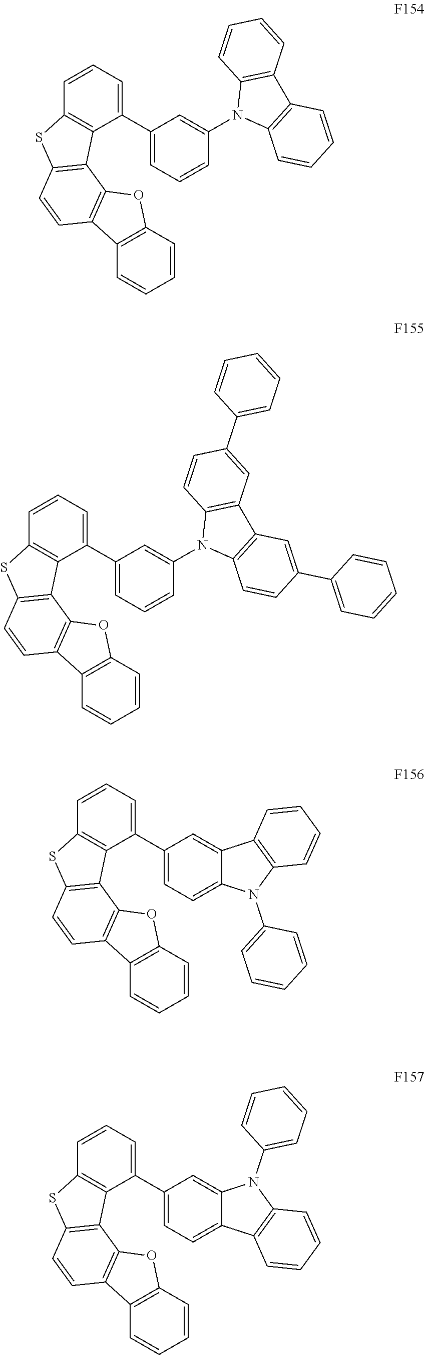 [SMILES: c1cc(-c2cccc3sc4ccc5c6ccccc6oc5c4c23)cc(-n2c3ccccc3c3ccccc32)c1 ; c1ccc(-c2ccc3c(c2)c2cc(-c4ccccc4)ccc2n3-c2cccc(-c3cccc4sc5ccc6c7ccccc7oc6c5c34)c2)cc1 ; c1ccc(-n2c3ccccc3c3cc(-c4cccc5sc6ccc7c8ccccc8oc7c6c45)ccc32)cc1 ; c1ccc(-n2c3ccccc3c3ccc(-c4cccc5sc6ccc7c8ccccc8oc7c6c45)cc32)cc1]

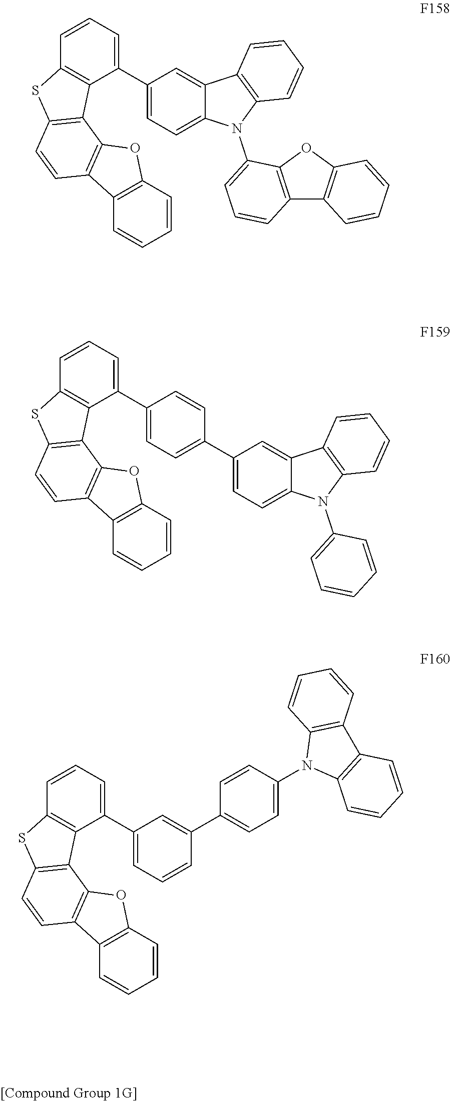 [SMILES: c1ccc2c(c1)oc1c(-n3c4ccccc4c4cc(-c5cccc6sc7ccc8c9ccccc9oc8c7c56)ccc43)cccc12 ; c1ccc(-n2c3ccccc3c3cc(-c4ccc(-c5cccc6sc7ccc8c9ccccc9oc8c7c56)cc4)ccc32)cc1 ; c1cc(-c2ccc(-n3c4ccccc4c4ccccc43)cc2)cc(-c2cccc3sc4ccc5c6ccccc6oc5c4c23)c1]

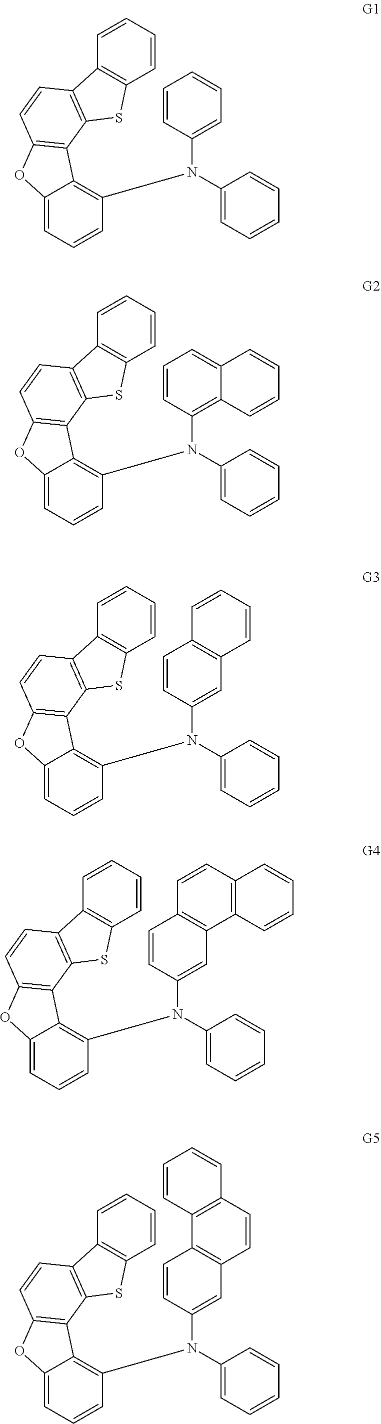 [SMILES: c1ccc(N(c2ccccc2)c2cccc3oc4ccc5c6ccccc6sc5c4c23)cc1 ; c1ccc(N(c2cccc3ccccc23)c2cccc3oc4ccc5c6ccccc6sc5c4c23)cc1 ; c1ccc(N(c2ccc3ccccc3c2)c2cccc3oc4ccc5c6ccccc6sc5c4c23)cc1 ; c1ccc(N(c2ccc3ccc4ccccc4c3c2)c2cccc3oc4ccc5c6ccccc6sc5c4c23)cc1 ; c1ccc(N(c2ccc3c(ccc4ccccc43)c2)c2cccc3oc4ccc5c6ccccc6sc5c4c23)cc1]

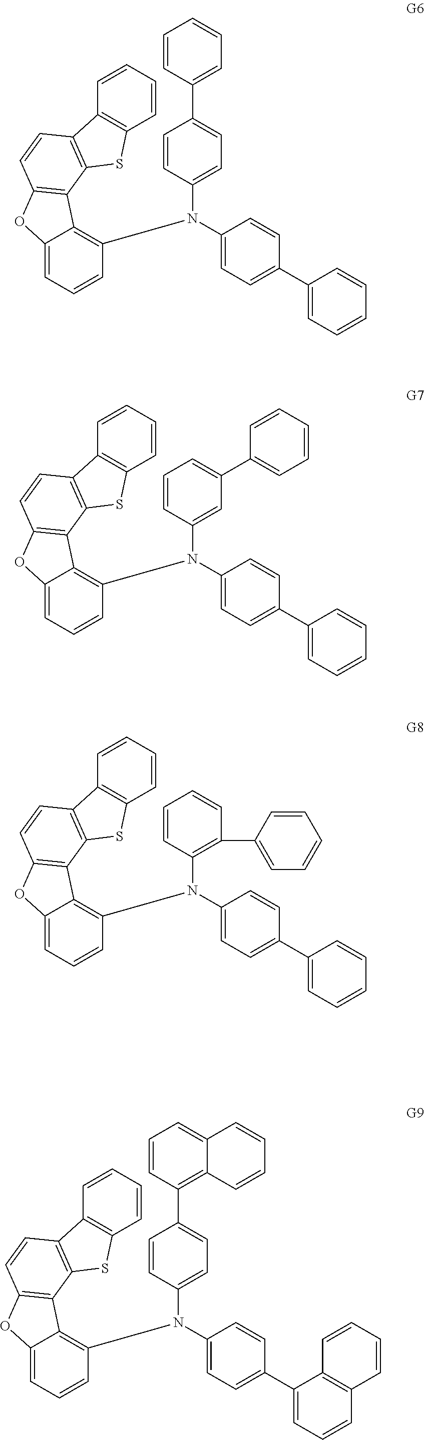 [SMILES: c1ccc(-c2ccc(N(c3ccc(-c4ccccc4)cc3)c3cccc4oc5ccc6c7ccccc7sc6c5c34)cc2)cc1 ; c1ccc(-c2ccc(N(c3cccc(-c4ccccc4)c3)c3cccc4oc5ccc6c7ccccc7sc6c5c34)cc2)cc1 ; c1ccc(-c2ccc(N(c3ccccc3-c3ccccc3)c3cccc4oc5ccc6c7ccccc7sc6c5c34)cc2)cc1 ; c1ccc2c(-c3ccc(N(c4ccc(-c5cccc6ccccc56)cc4)c4cccc5oc6ccc7c8ccccc8sc7c6c45)cc3)cccc2c1]

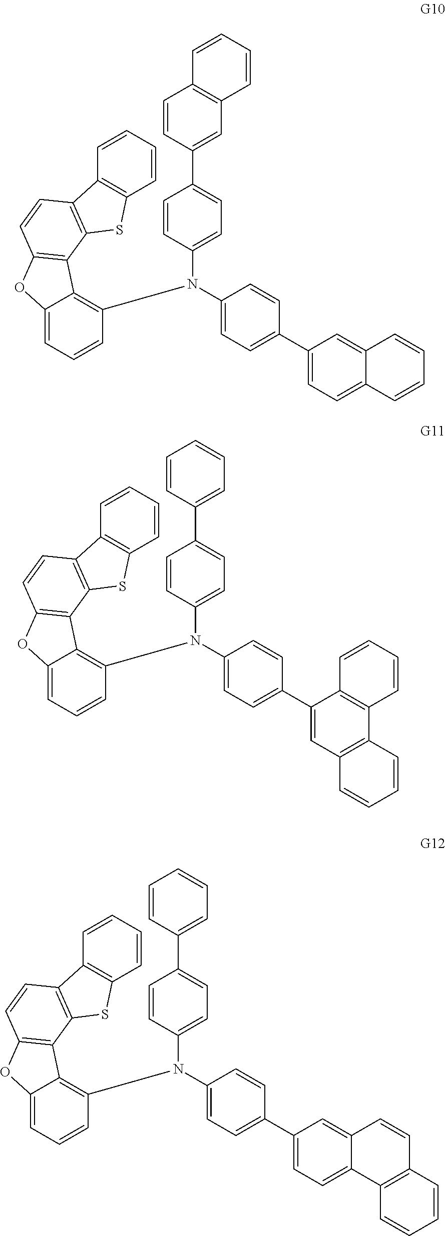 [SMILES: c1ccc2cc(-c3ccc(N(c4ccc(-c5ccc6ccccc6c5)cc4)c4cccc5oc6ccc7c8ccccc8sc7c6c45)cc3)ccc2c1 ; c1ccc(-c2ccc(N(c3ccc(-c4cc5ccccc5c5ccccc45)cc3)c3cccc4oc5ccc6c7ccccc7sc6c5c34)cc2)cc1 ; c1ccc(-c2ccc(N(c3ccc(-c4ccc5c(ccc6ccccc65)c4)cc3)c3cccc4oc5ccc6c7ccccc7sc6c5c34)cc2)cc1]

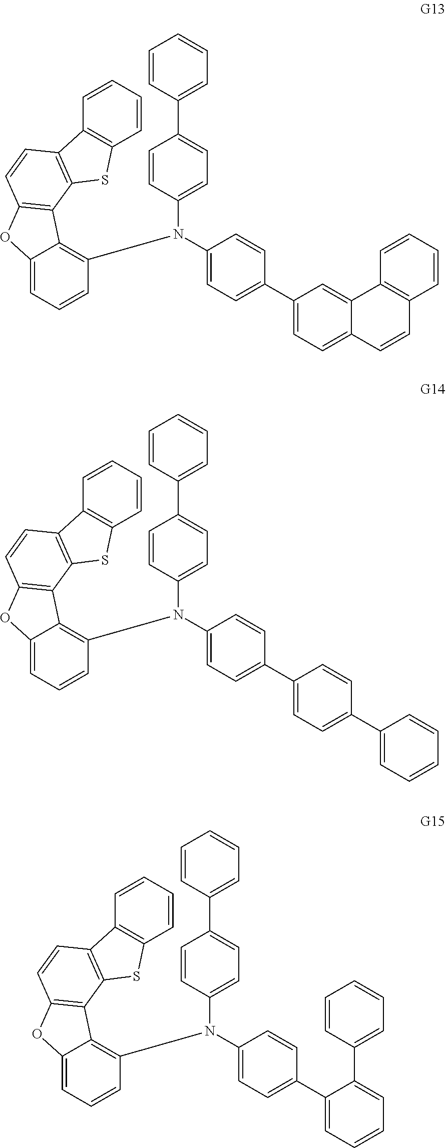 [SMILES: c1ccc(-c2ccc(N(c3ccc(-c4ccc5ccc6ccccc6c5c4)cc3)c3cccc4oc5ccc6c7ccccc7sc6c5c34)cc2)cc1 ; c1ccc(-c2ccc(-c3ccc(N(c4ccc(-c5ccccc5)cc4)c4cccc5oc6ccc7c8ccccc8sc7c6c45)cc3)cc2)cc1 ; c1ccc(-c2ccc(N(c3ccc(-c4ccccc4-c4ccccc4)cc3)c3cccc4oc5ccc6c7ccccc7sc6c5c34)cc2)cc1]

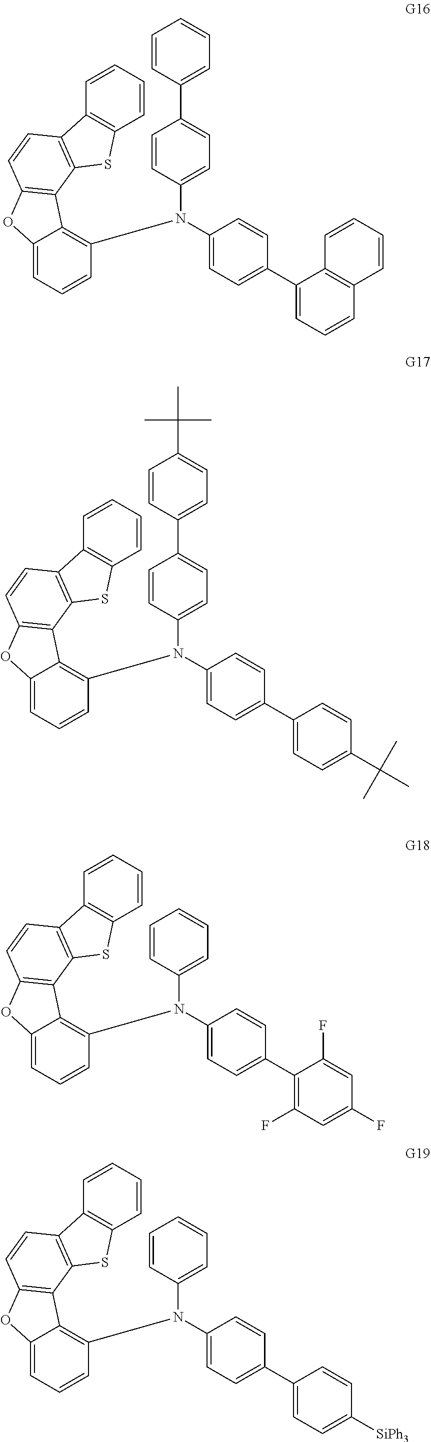

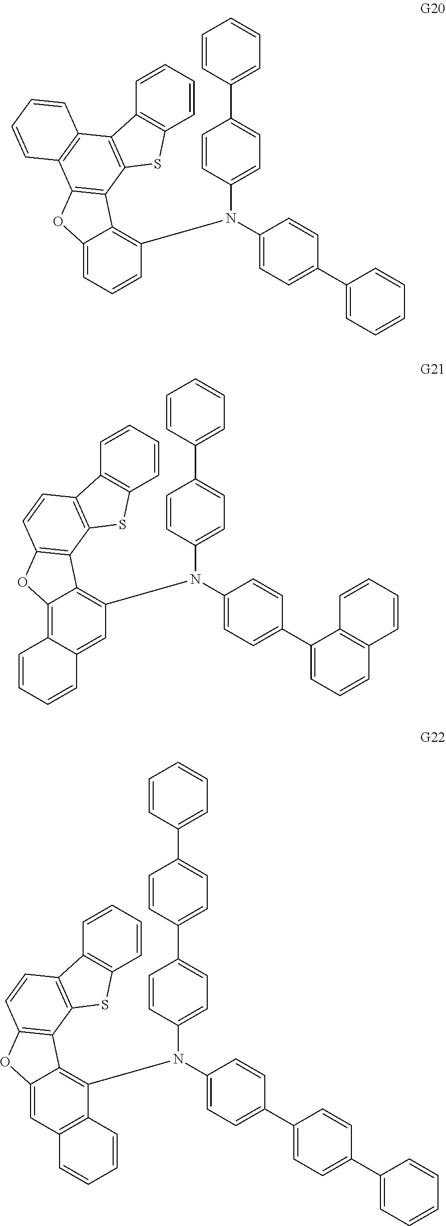 [SMILES: c1ccc(-c2ccc(N(c3ccc(-c4ccccc4)cc3)c3cccc4oc5c6ccccc6c6c7ccccc7sc6c5c34)cc2)cc1 ; c1ccc(-c2ccc(N(c3ccc(-c4cccc5ccccc45)cc3)c3cc4ccccc4c4oc5ccc6c7ccccc7sc6c5c34)cc2)cc1 ; c1ccc(-c2ccc(-c3ccc(N(c4ccc(-c5ccc(-c6ccccc6)cc5)cc4)c4c5ccccc5cc5oc6ccc7c8ccccc8sc7c6c45)cc3)cc2)cc1]

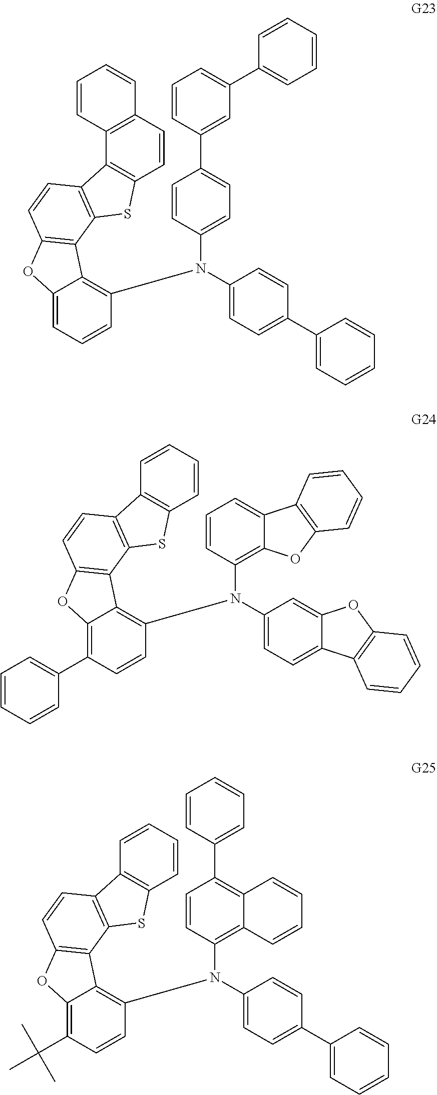 [SMILES: c1ccc(-c2ccc(N(c3ccc(-c4cccc(-c5ccccc5)c4)cc3)c3cccc4oc5ccc6c(sc7ccc8ccccc8c76)c5c34)cc2)cc1 ; c1ccc(-c2ccc(N(c3ccc4c(c3)oc3ccccc34)c3cccc4c3oc3ccccc34)c3c2oc2ccc4c5ccccc5sc4c23)cc1 ; CC(C)(C)c1ccc(N(c2ccc(-c3ccccc3)cc2)c2ccc(-c3ccccc3)c3ccccc23)c2c1oc1ccc3c4ccccc4sc3c12]

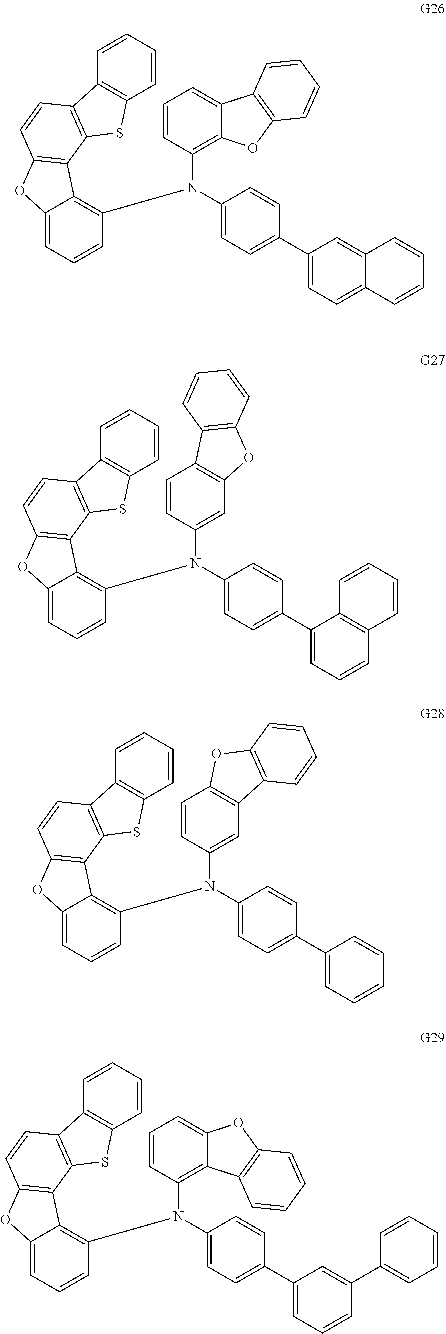 [SMILES: c1ccc2cc(-c3ccc(N(c4cccc5c4oc4ccccc45)c4cccc5oc6ccc7c8ccccc8sc7c6c45)cc3)ccc2c1 ; c1ccc2c(-c3ccc(N(c4ccc5c(c4)oc4ccccc45)c4cccc5oc6ccc7c8ccccc8sc7c6c45)cc3)cccc2c1 ; c1ccc(-c2ccc(N(c3ccc4oc5ccccc5c4c3)c3cccc4oc5ccc6c7ccccc7sc6c5c34)cc2)cc1 ; c1ccc(-c2cccc(-c3ccc(N(c4cccc5oc6ccccc6c45)c4cccc5oc6ccc7c8ccccc8sc7c6c45)cc3)c2)cc1]

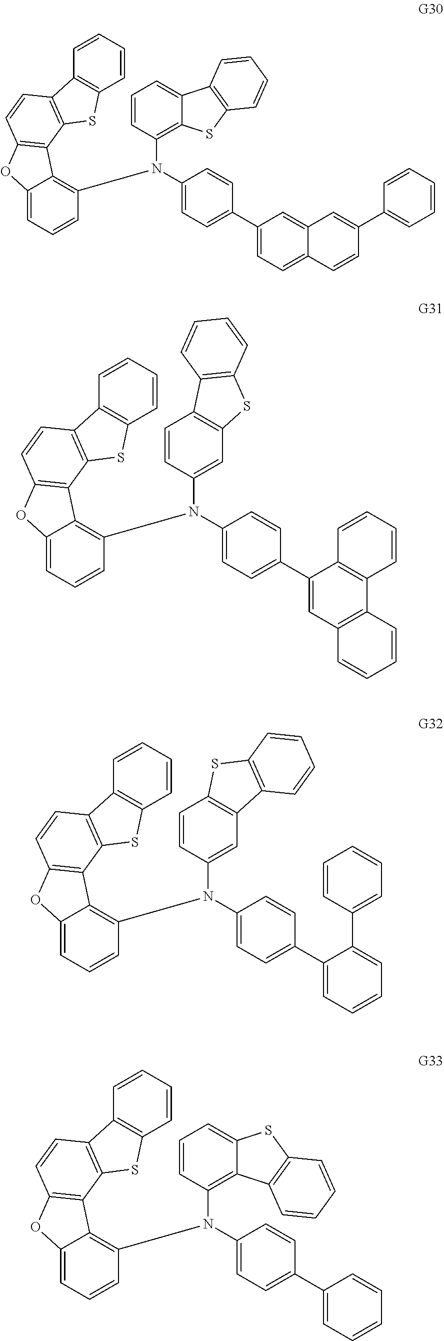 [SMILES: c1ccc(-c2ccc3ccc(-c4ccc(N(c5cccc6c5sc5ccccc56)c5cccc6oc7ccc8c9ccccc9sc8c7c56)cc4)cc3c2)cc1 ; c1ccc2c(c1)cc(-c1ccc(N(c3ccc4c(c3)sc3ccccc34)c3cccc4oc5ccc6c7ccccc7sc6c5c34)cc1)c1ccccc12 ; c1ccc(-c2ccccc2-c2ccc(N(c3ccc4sc5ccccc5c4c3)c3cccc4oc5ccc6c7ccccc7sc6c5c34)cc2)cc1 ; c1ccc(-c2ccc(N(c3cccc4sc5ccccc5c34)c3cccc4oc5ccc6c7ccccc7sc6c5c34)cc2)cc1]

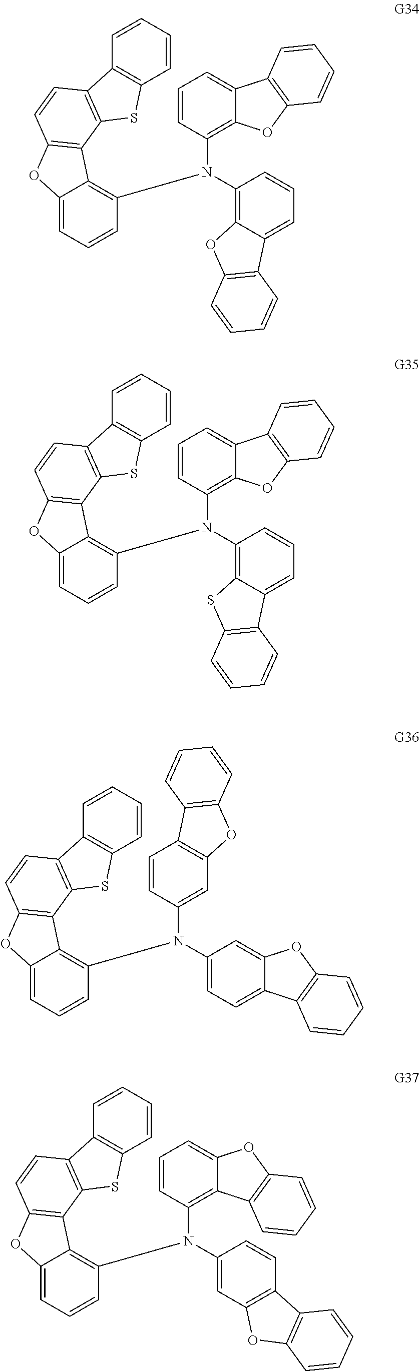 [SMILES: c1ccc2c(c1)oc1c(N(c3cccc4c3oc3ccccc34)c3cccc4oc5ccc6c7ccccc7sc6c5c34)cccc12 ; c1ccc2c(c1)oc1c(N(c3cccc4c3sc3ccccc34)c3cccc4oc5ccc6c7ccccc7sc6c5c34)cccc12 ; c1ccc2c(c1)oc1cc(N(c3ccc4c(c3)oc3ccccc34)c3cccc4oc5ccc6c7ccccc7sc6c5c34)ccc12 ; c1ccc2c(c1)oc1cc(N(c3cccc4oc5ccccc5c34)c3cccc4oc5ccc6c7ccccc7sc6c5c34)ccc12]

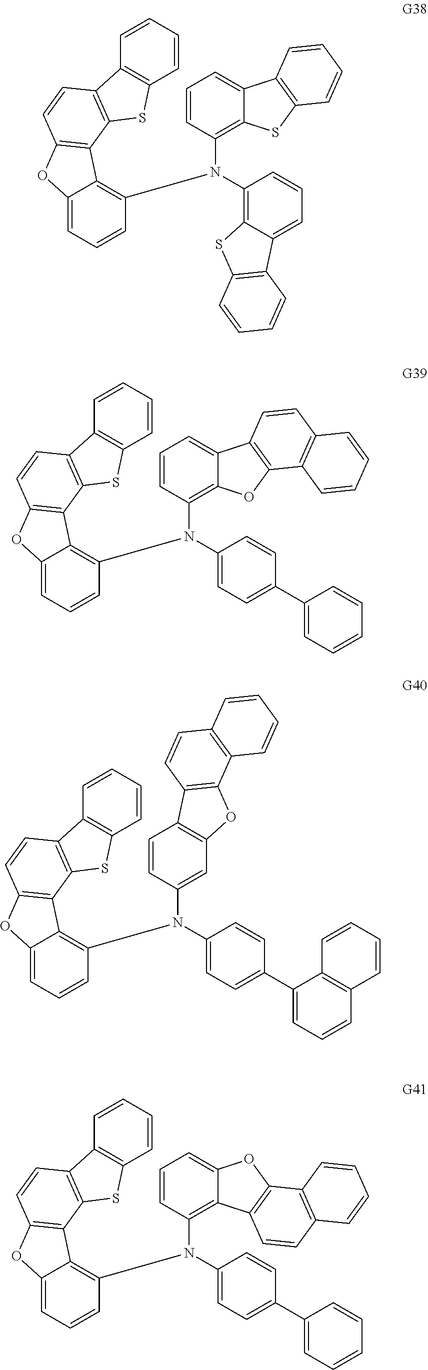 [SMILES: c1cc(N(c2cccc3c2sc2ccccc23)c2cccc3c2sc2ccccc23)c2c(c1)oc1ccc3c4ccccc4sc3c12 ; c1ccc(-c2ccc(N(c3cccc4c3oc3c5ccccc5ccc43)c3cccc4oc5ccc6c7ccccc7sc6c5c34)cc2)cc1 ; c1ccc2c(-c3ccc(N(c4ccc5c(c4)oc4c6ccccc6ccc54)c4cccc5oc6ccc7c8ccccc8sc7c6c45)cc3)cccc2c1 ; c1ccc(-c2ccc(N(c3cccc4oc5c6ccccc6ccc5c34)c3cccc4oc5ccc6c7ccccc7sc6c5c34)cc2)cc1]

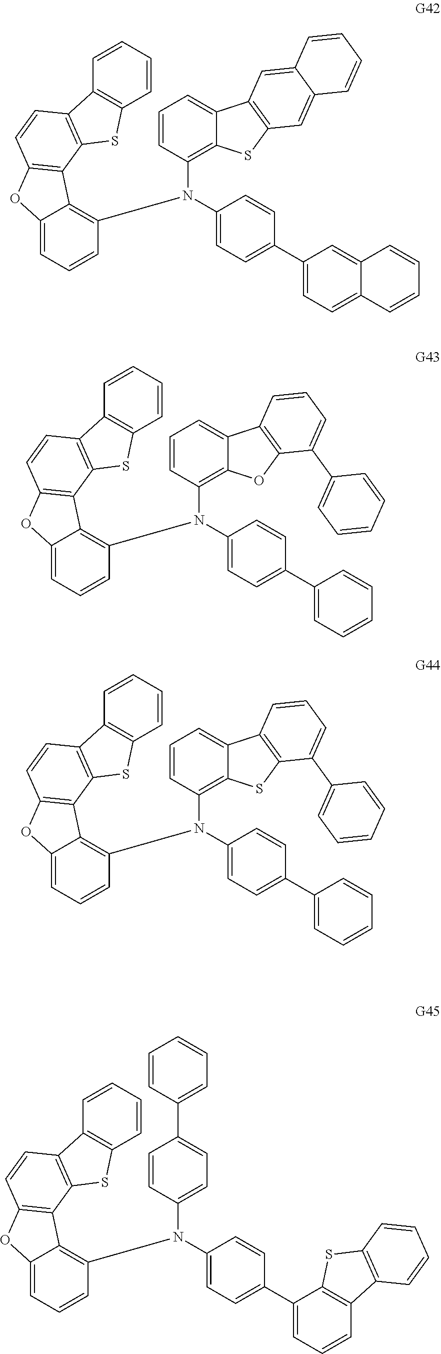 [SMILES: c1ccc2cc(-c3ccc(N(c4cccc5c4sc4cc6ccccc6cc45)c4cccc5oc6ccc7c8ccccc8sc7c6c45)cc3)ccc2c1 ; c1ccc(-c2ccc(N(c3cccc4c3oc3c(-c5ccccc5)cccc34)c3cccc4oc5ccc6c7ccccc7sc6c5c34)cc2)cc1 ; c1ccc(-c2ccc(N(c3cccc4c3sc3c(-c5ccccc5)cccc34)c3cccc4oc5ccc6c7ccccc7sc6c5c34)cc2)cc1 ; c1ccc(-c2ccc(N(c3ccc(-c4cccc5c4sc4ccccc45)cc3)c3cccc4oc5ccc6c7ccccc7sc6c5c34)cc2)cc1]

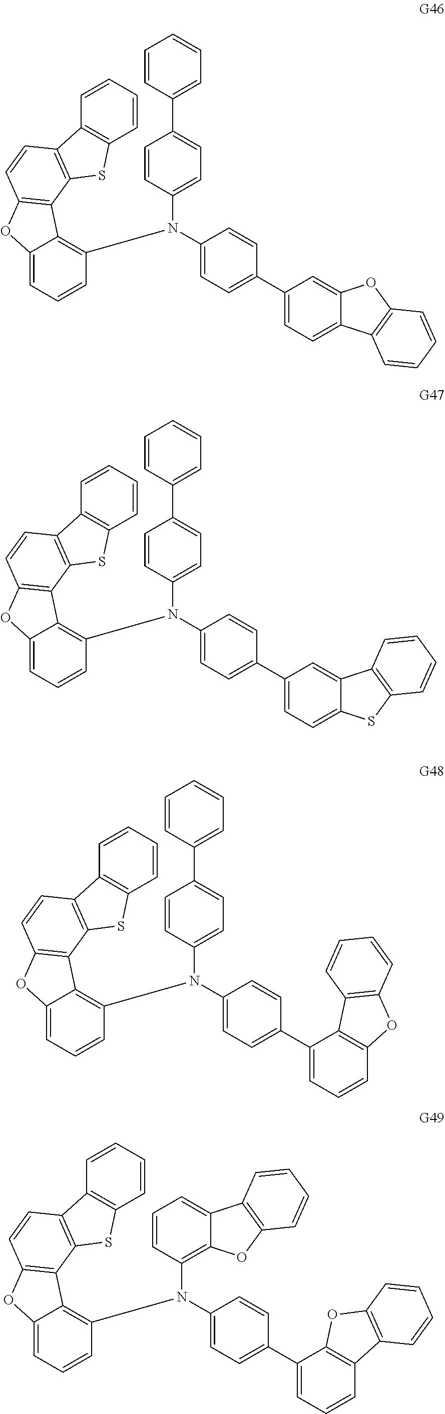 [SMILES: c1ccc(-c2ccc(N(c3ccc(-c4ccc5c(c4)oc4ccccc45)cc3)c3cccc4oc5ccc6c7ccccc7sc6c5c34)cc2)cc1 ; c1ccc(-c2ccc(N(c3ccc(-c4ccc5sc6ccccc6c5c4)cc3)c3cccc4oc5ccc6c7ccccc7sc6c5c34)cc2)cc1 ; c1ccc(-c2ccc(N(c3ccc(-c4cccc5oc6ccccc6c45)cc3)c3cccc4oc5ccc6c7ccccc7sc6c5c34)cc2)cc1 ; c1ccc2c(c1)oc1c(-c3ccc(N(c4cccc5c4oc4ccccc45)c4cccc5oc6ccc7c8ccccc8sc7c6c45)cc3)cccc12]

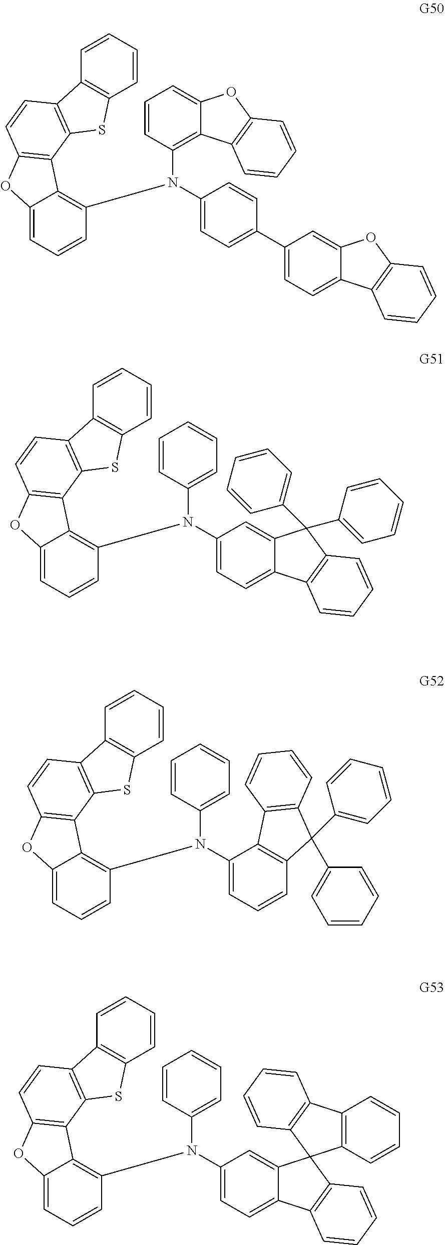 [SMILES: c1ccc2c(c1)oc1cc(-c3ccc(N(c4cccc5oc6ccccc6c45)c4cccc5oc6ccc7c8ccccc8sc7c6c45)cc3)ccc12 ; c1ccc(N(c2ccc3c(c2)C(c2ccccc2)(c2ccccc2)c2ccccc2-3)c2cccc3oc4ccc5c6ccccc6sc5c4c23)cc1 ; c1ccc(N(c2cccc3c2-c2ccccc2C3(c2ccccc2)c2ccccc2)c2cccc3oc4ccc5c6ccccc6sc5c4c23)cc1 ; c1ccc(N(c2ccc3c(c2)C2(c4ccccc4-c4ccccc42)c2ccccc2-3)c2cccc3oc4ccc5c6ccccc6sc5c4c23)cc1]

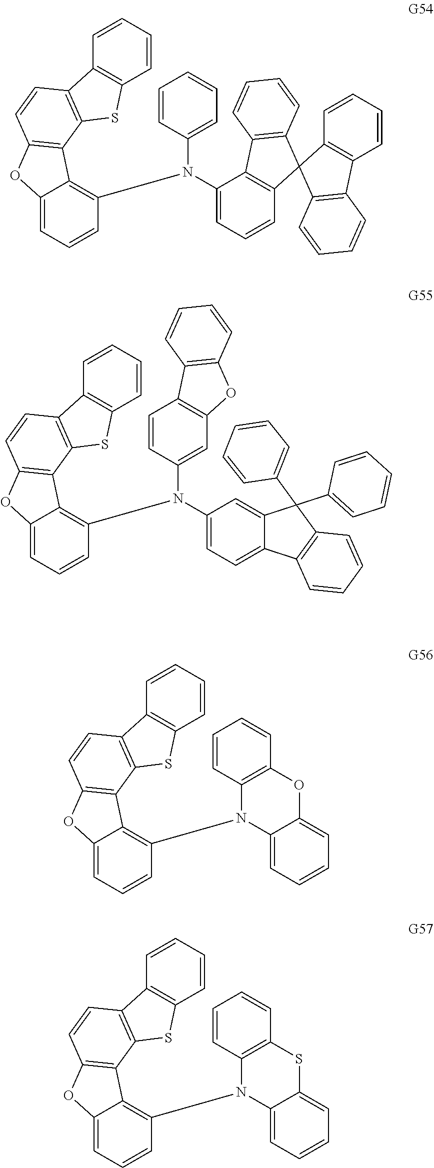 [SMILES: c1ccc(N(c2cccc3c2-c2ccccc2C32c3ccccc3-c3ccccc32)c2cccc3oc4ccc5c6ccccc6sc5c4c23)cc1 ; c1ccc(C2(c3ccccc3)c3ccccc3-c3ccc(N(c4ccc5c(c4)oc4ccccc45)c4cccc5oc6ccc7c8ccccc8sc7c6c45)cc32)cc1 ; c1ccc2c(c1)Oc1ccccc1N2c1cccc2oc3ccc4c5ccccc5sc4c3c12 ; c1ccc2c(c1)Sc1ccccc1N2c1cccc2oc3ccc4c5ccccc5sc4c3c12]

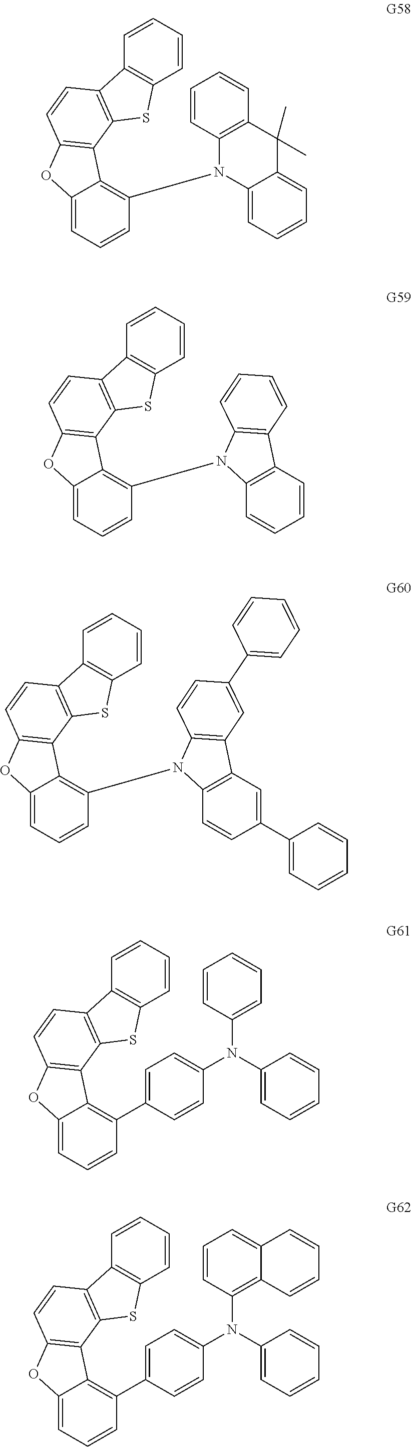 [SMILES: CC1(C)c2ccccc2N(c2cccc3oc4ccc5c6ccccc6sc5c4c23)c2ccccc21 ; c1cc(-n2c3ccccc3c3ccccc32)c2c(c1)oc1ccc3c4ccccc4sc3c12 ; c1ccc(-c2ccc3c(c2)c2cc(-c4ccccc4)ccc2n3-c2cccc3oc4ccc5c6ccccc6sc5c4c23)cc1 ; c1ccc(N(c2ccccc2)c2ccc(-c3cccc4oc5ccc6c7ccccc7sc6c5c34)cc2)cc1 ; c1ccc(N(c2ccc(-c3cccc4oc5ccc6c7ccccc7sc6c5c34)cc2)c2cccc3ccccc23)cc1]

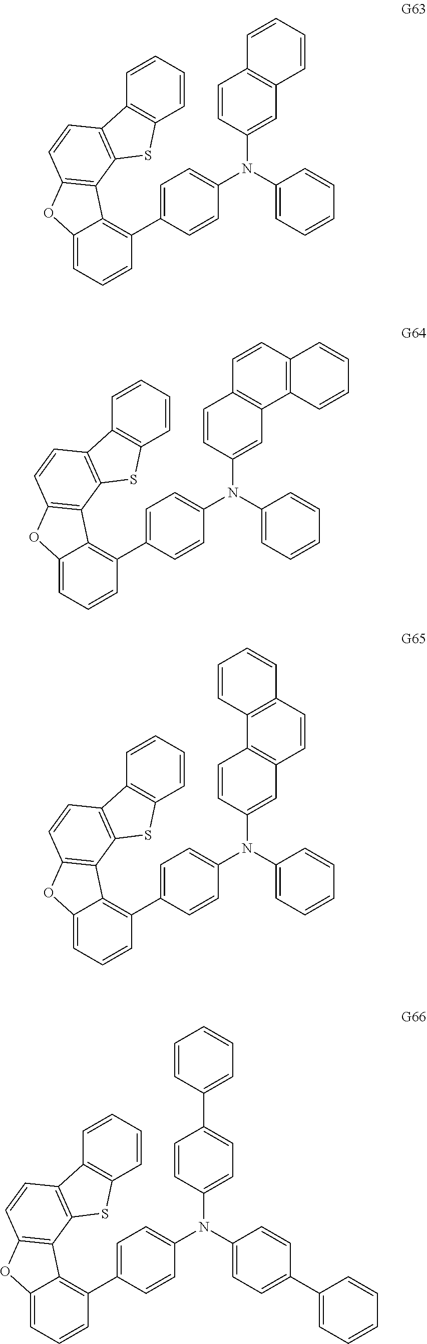 [SMILES: c1ccc(N(c2ccc(-c3cccc4oc5ccc6c7ccccc7sc6c5c34)cc2)c2ccc3ccccc3c2)cc1 ; c1ccc(N(c2ccc(-c3cccc4oc5ccc6c7ccccc7sc6c5c34)cc2)c2ccc3ccc4ccccc4c3c2)cc1 ; c1ccc(N(c2ccc(-c3cccc4oc5ccc6c7ccccc7sc6c5c34)cc2)c2ccc3c(ccc4ccccc43)c2)cc1 ; c1ccc(-c2ccc(N(c3ccc(-c4ccccc4)cc3)c3ccc(-c4cccc5oc6ccc7c8ccccc8sc7c6c45)cc3)cc2)cc1]

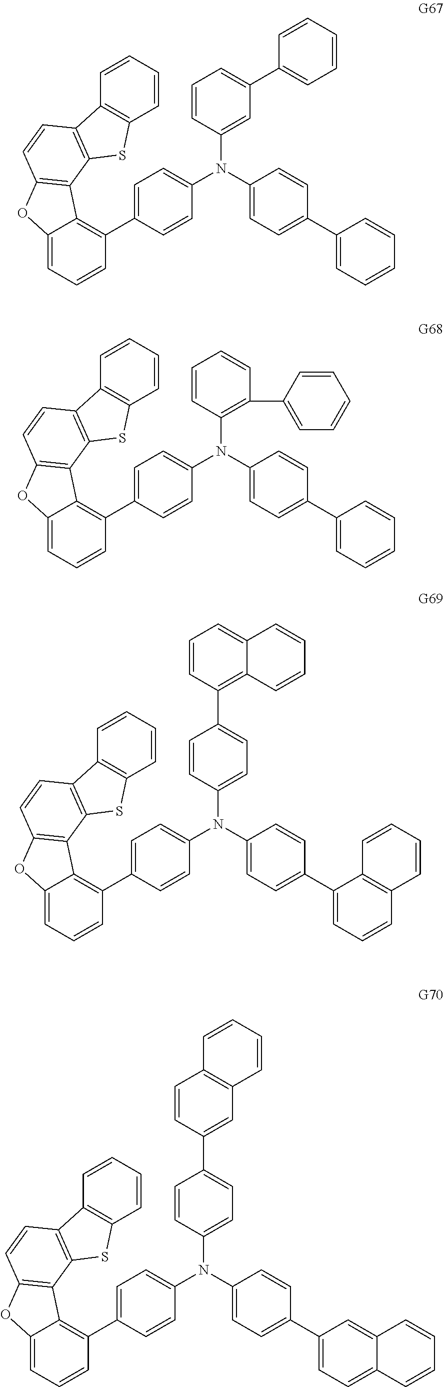 [SMILES: c1ccc(-c2ccc(N(c3ccc(-c4cccc5oc6ccc7c8ccccc8sc7c6c45)cc3)c3cccc(-c4ccccc4)c3)cc2)cc1 ; c1ccc(-c2ccc(N(c3ccc(-c4cccc5oc6ccc7c8ccccc8sc7c6c45)cc3)c3ccccc3-c3ccccc3)cc2)cc1 ; c1ccc2c(-c3ccc(N(c4ccc(-c5cccc6ccccc56)cc4)c4ccc(-c5cccc6oc7ccc8c9ccccc9sc8c7c56)cc4)cc3)cccc2c1 ; c1ccc2cc(-c3ccc(N(c4ccc(-c5ccc6ccccc6c5)cc4)c4ccc(-c5cccc6oc7ccc8c9ccccc9sc8c7c56)cc4)cc3)ccc2c1]

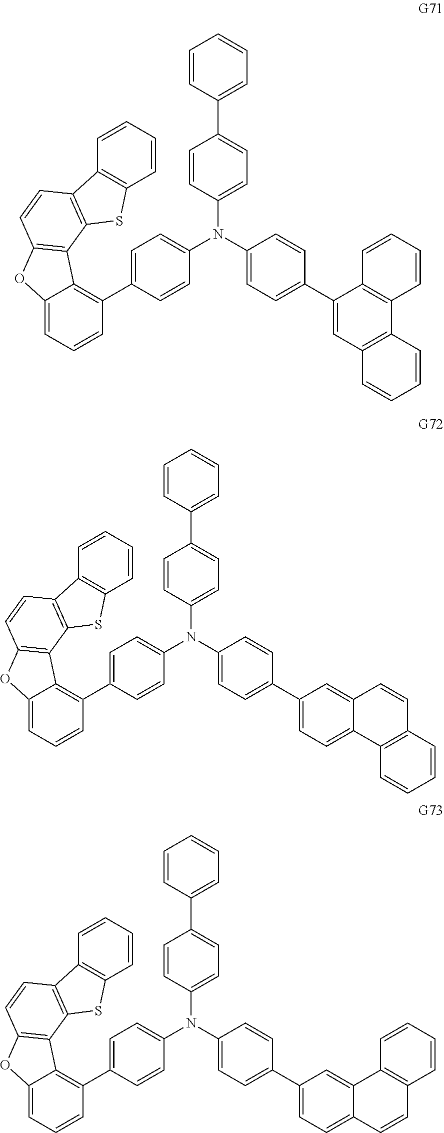 [SMILES: c1ccc(-c2ccc(N(c3ccc(-c4cc5ccccc5c5ccccc45)cc3)c3ccc(-c4cccc5oc6ccc7c8ccccc8sc7c6c45)cc3)cc2)cc1 ; c1ccc(-c2ccc(N(c3ccc(-c4ccc5c(ccc6ccccc65)c4)cc3)c3ccc(-c4cccc5oc6ccc7c8ccccc8sc7c6c45)cc3)cc2)cc1 ; c1ccc(-c2ccc(N(c3ccc(-c4ccc5ccc6ccccc6c5c4)cc3)c3ccc(-c4cccc5oc6ccc7c8ccccc8sc7c6c45)cc3)cc2)cc1]

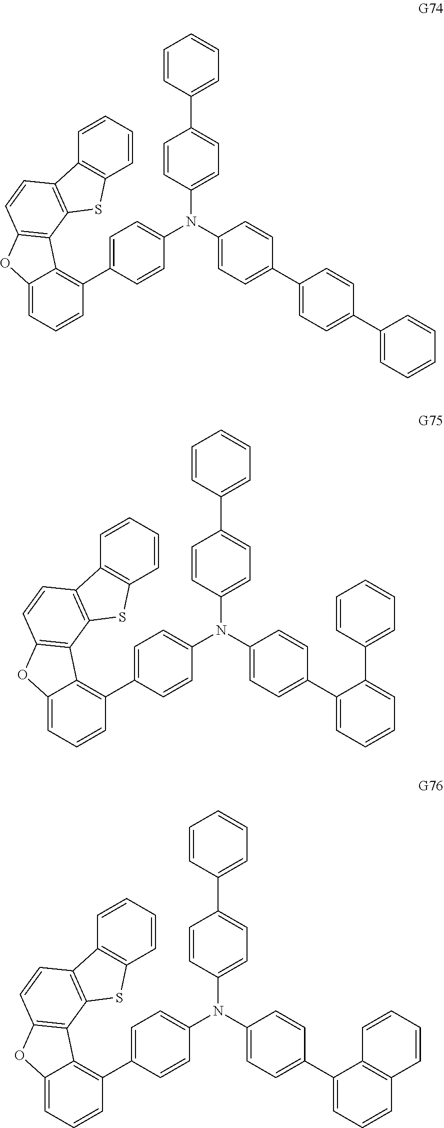 [SMILES: c1ccc(-c2ccc(-c3ccc(N(c4ccc(-c5ccccc5)cc4)c4ccc(-c5cccc6oc7ccc8c9ccccc9sc8c7c56)cc4)cc3)cc2)cc1 ; c1ccc(-c2ccc(N(c3ccc(-c4ccccc4-c4ccccc4)cc3)c3ccc(-c4cccc5oc6ccc7c8ccccc8sc7c6c45)cc3)cc2)cc1 ; c1ccc(-c2ccc(N(c3ccc(-c4cccc5ccccc45)cc3)c3ccc(-c4cccc5oc6ccc7c8ccccc8sc7c6c45)cc3)cc2)cc1]

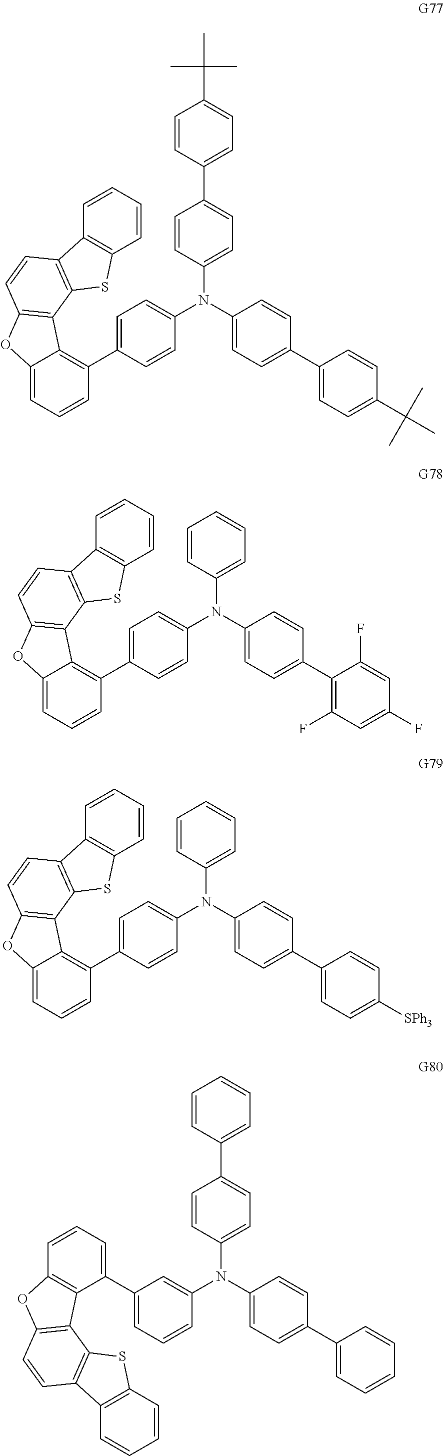

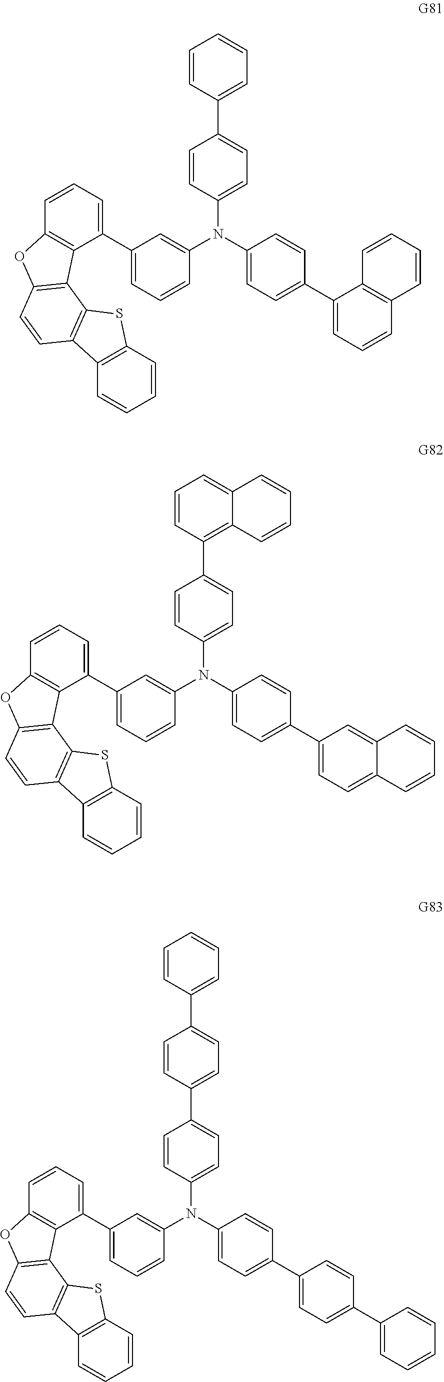 [SMILES: c1ccc(-c2ccc(N(c3ccc(-c4cccc5ccccc45)cc3)c3cccc(-c4cccc5oc6ccc7c8ccccc8sc7c6c45)c3)cc2)cc1 ; c1cc(-c2cccc3oc4ccc5c6ccccc6sc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6oc7ccc8c9ccccc9sc8c7c56)c4)cc3)cc2)cc1]

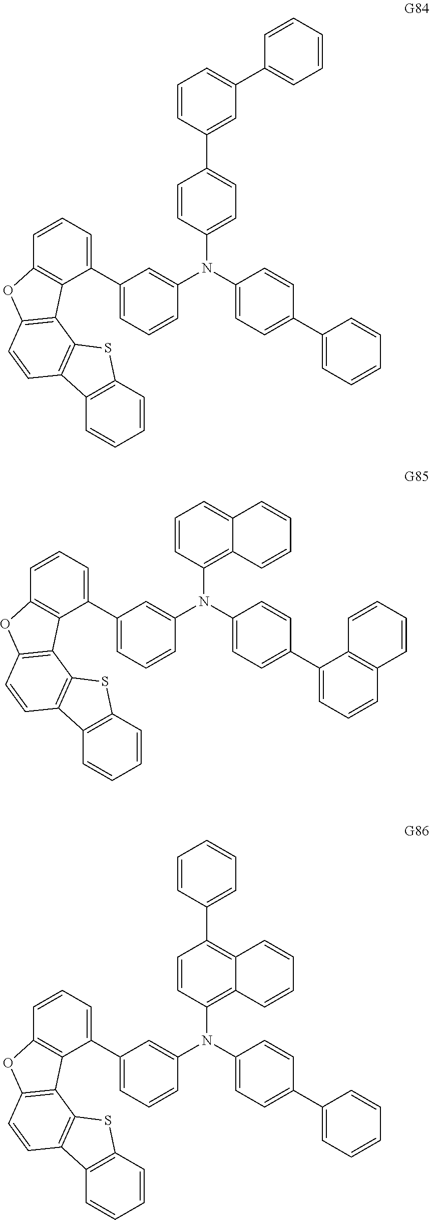 [SMILES: c1ccc(-c2ccc(N(c3ccc(-c4cccc(-c5ccccc5)c4)cc3)c3cccc(-c4cccc5oc6ccc7c8ccccc8sc7c6c45)c3)cc2)cc1 ; c1cc(-c2cccc3oc4ccc5c6ccccc6sc5c4c23)cc(N(c2ccc(-c3cccc4ccccc34)cc2)c2cccc3ccccc23)c1 ; c1ccc(-c2ccc(N(c3cccc(-c4cccc5oc6ccc7c8ccccc8sc7c6c45)c3)c3ccc(-c4ccccc4)c4ccccc34)cc2)cc1]

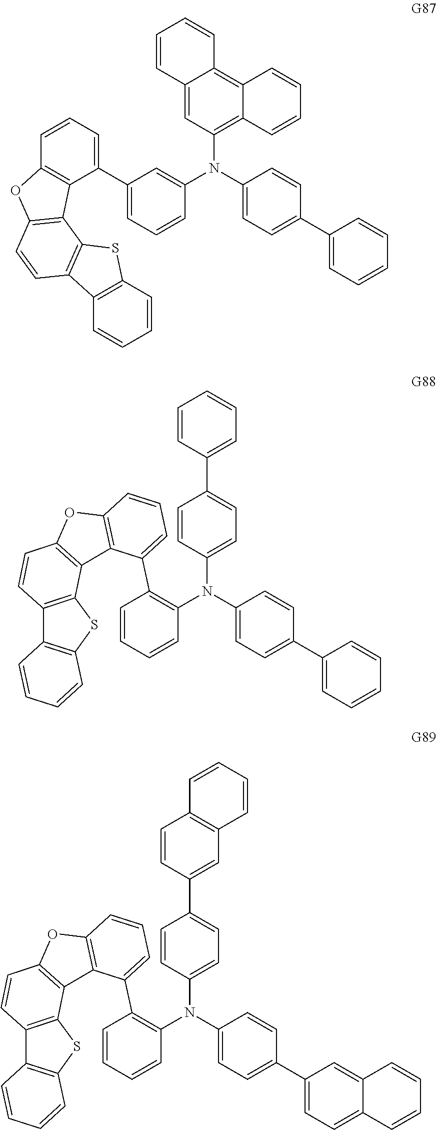 [SMILES: c1ccc(-c2ccc(N(c3cccc(-c4cccc5oc6ccc7c8ccccc8sc7c6c45)c3)c3cc4ccccc4c4ccccc34)cc2)cc1 ; c1ccc(-c2ccc(N(c3ccc(-c4ccccc4)cc3)c3ccccc3-c3cccc4oc5ccc6c7ccccc7sc6c5c34)cc2)cc1 ; c1ccc(N(c2ccc(-c3ccc4ccccc4c3)cc2)c2ccc(-c3ccc4ccccc4c3)cc2)c(-c2cccc3oc4ccc5c6ccccc6sc5c4c23)c1]

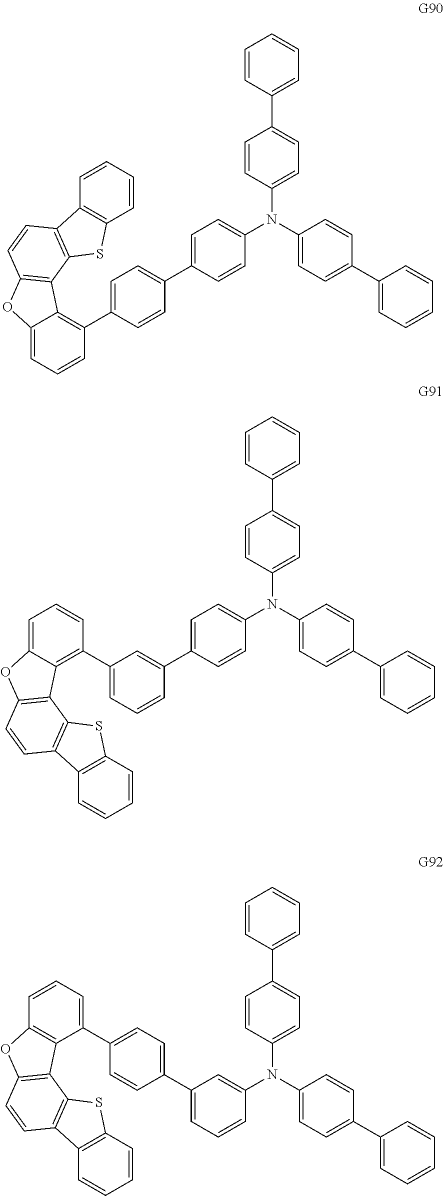 [SMILES: c1ccc(-c2ccc(N(c3ccc(-c4ccccc4)cc3)c3ccc(-c4ccc(-c5cccc6oc7ccc8c9ccccc9sc8c7c56)cc4)cc3)cc2)cc1 ; c1ccc(-c2ccc(N(c3ccc(-c4ccccc4)cc3)c3ccc(-c4cccc(-c5cccc6oc7ccc8c9ccccc9sc8c7c56)c4)cc3)cc2)cc1 ; c1ccc(-c2ccc(N(c3ccc(-c4ccccc4)cc3)c3cccc(-c4ccc(-c5cccc6oc7ccc8c9ccccc9sc8c7c56)cc4)c3)cc2)cc1]

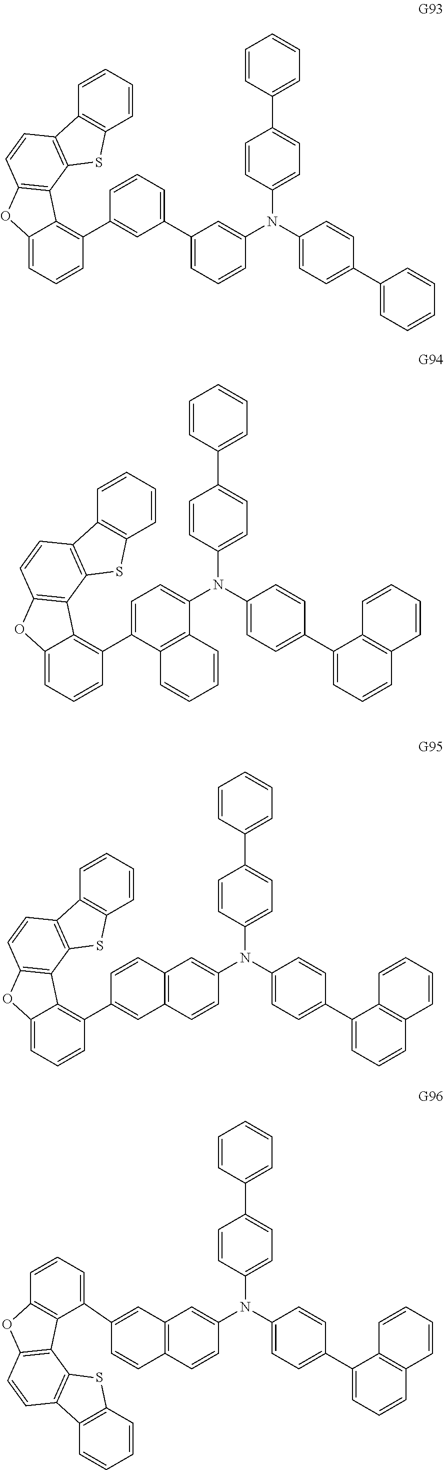 [SMILES: c1ccc(-c2ccc(N(c3ccc(-c4ccccc4)cc3)c3cccc(-c4cccc(-c5cccc6oc7ccc8c9ccccc9sc8c7c56)c4)c3)cc2)cc1 ; c1ccc(-c2ccc(N(c3ccc(-c4cccc5ccccc45)cc3)c3ccc(-c4cccc5oc6ccc7c8ccccc8sc7c6c45)c4ccccc34)cc2)cc1 ; c1ccc(-c2ccc(N(c3ccc(-c4cccc5ccccc45)cc3)c3ccc4cc(-c5cccc6oc7ccc8c9ccccc9sc8c7c56)ccc4c3)cc2)cc1 ; c1ccc(-c2ccc(N(c3ccc(-c4cccc5ccccc45)cc3)c3ccc4ccc(-c5cccc6oc7ccc8c9ccccc9sc8c7c56)cc4c3)cc2)cc1]

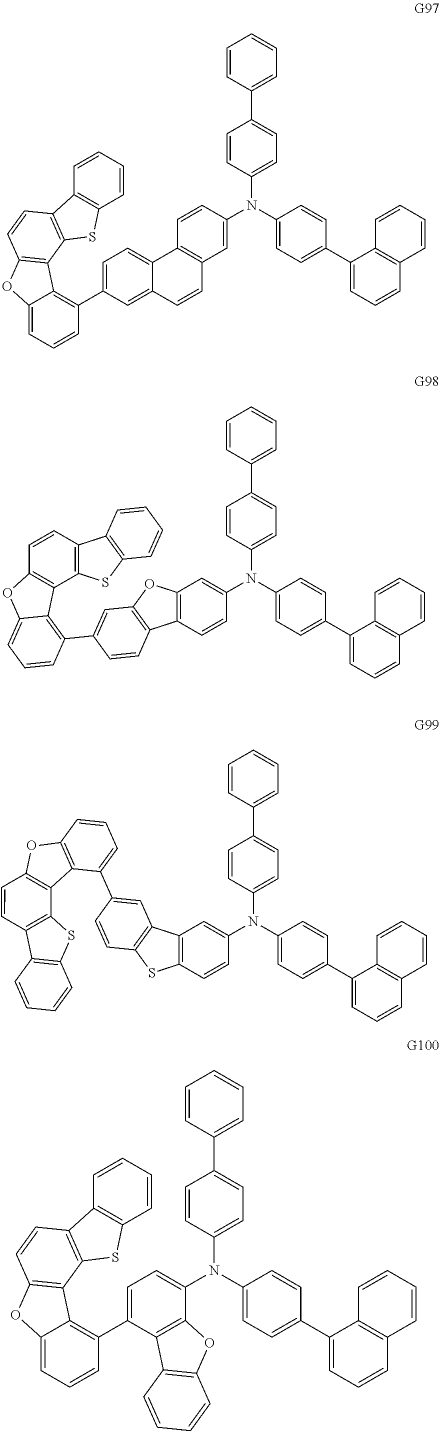 [SMILES: c1ccc(-c2ccc(N(c3ccc(-c4cccc5ccccc45)cc3)c3ccc4c(ccc5cc(-c6cccc7oc8ccc9c%10ccccc%10sc9c8c67)ccc54)c3)cc2)cc1 ; c1ccc(-c2ccc(N(c3ccc(-c4cccc5ccccc45)cc3)c3ccc4c(c3)oc3cc(-c5cccc6oc7ccc8c9ccccc9sc8c7c56)ccc34)cc2)cc1 ; c1ccc(-c2ccc(N(c3ccc(-c4cccc5ccccc45)cc3)c3ccc4sc5ccc(-c6cccc7oc8ccc9c%10ccccc%10sc9c8c67)cc5c4c3)cc2)cc1 ; c1ccc(-c2ccc(N(c3ccc(-c4cccc5ccccc45)cc3)c3ccc(-c4cccc5oc6ccc7c8ccccc8sc7c6c45)c4c3oc3ccccc34)cc2)cc1]

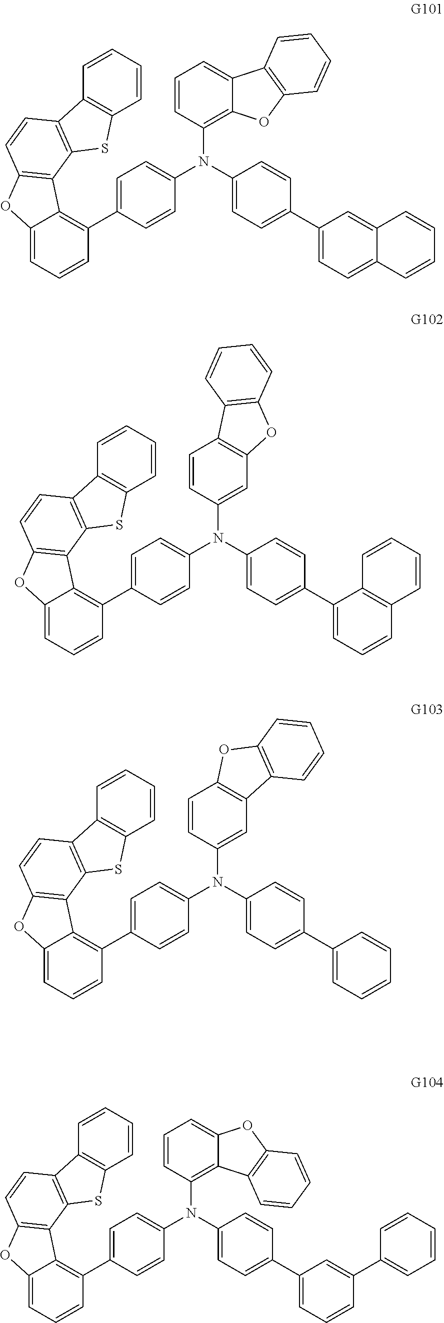 [SMILES: c1ccc2cc(-c3ccc(N(c4ccc(-c5cccc6oc7ccc8c9ccccc9sc8c7c56)cc4)c4cccc5c4oc4ccccc45)cc3)ccc2c1 ; c1ccc2c(-c3ccc(N(c4ccc(-c5cccc6oc7ccc8c9ccccc9sc8c7c56)cc4)c4ccc5c(c4)oc4ccccc45)cc3)cccc2c1 ; c1ccc(-c2ccc(N(c3ccc(-c4cccc5oc6ccc7c8ccccc8sc7c6c45)cc3)c3ccc4oc5ccccc5c4c3)cc2)cc1 ; c1ccc(-c2cccc(-c3ccc(N(c4ccc(-c5cccc6oc7ccc8c9ccccc9sc8c7c56)cc4)c4cccc5oc6ccccc6c45)cc3)c2)cc1]

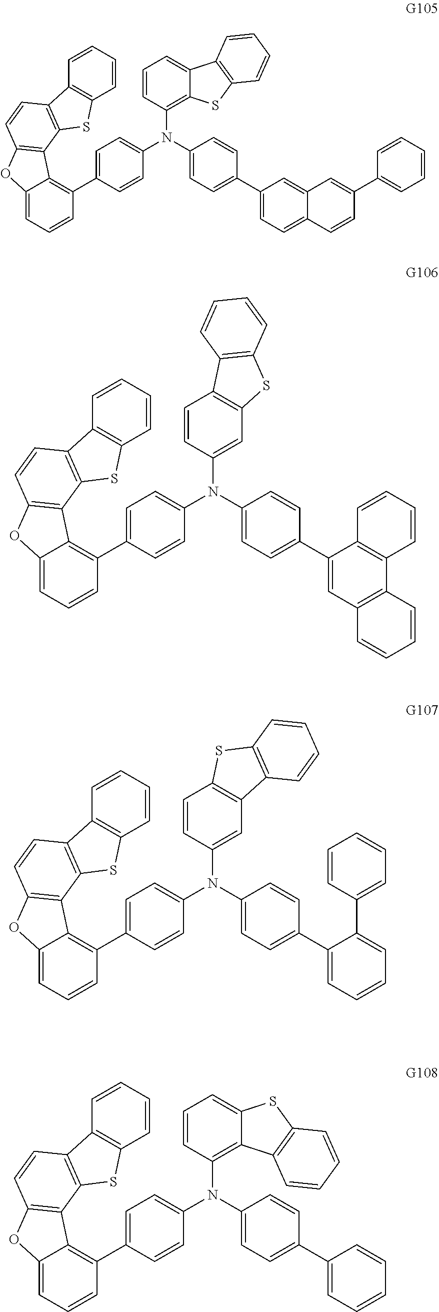 [SMILES: c1ccc(-c2ccc3ccc(-c4ccc(N(c5ccc(-c6cccc7oc8ccc9c%10ccccc%10sc9c8c67)cc5)c5cccc6c5sc5ccccc56)cc4)cc3c2)cc1 ; c1ccc2c(c1)cc(-c1ccc(N(c3ccc(-c4cccc5oc6ccc7c8ccccc8sc7c6c45)cc3)c3ccc4c(c3)sc3ccccc34)cc1)c1ccccc12 ; c1ccc(-c2ccccc2-c2ccc(N(c3ccc(-c4cccc5oc6ccc7c8ccccc8sc7c6c45)cc3)c3ccc4sc5ccccc5c4c3)cc2)cc1 ; c1ccc(-c2ccc(N(c3ccc(-c4cccc5oc6ccc7c8ccccc8sc7c6c45)cc3)c3cccc4sc5ccccc5c34)cc2)cc1]

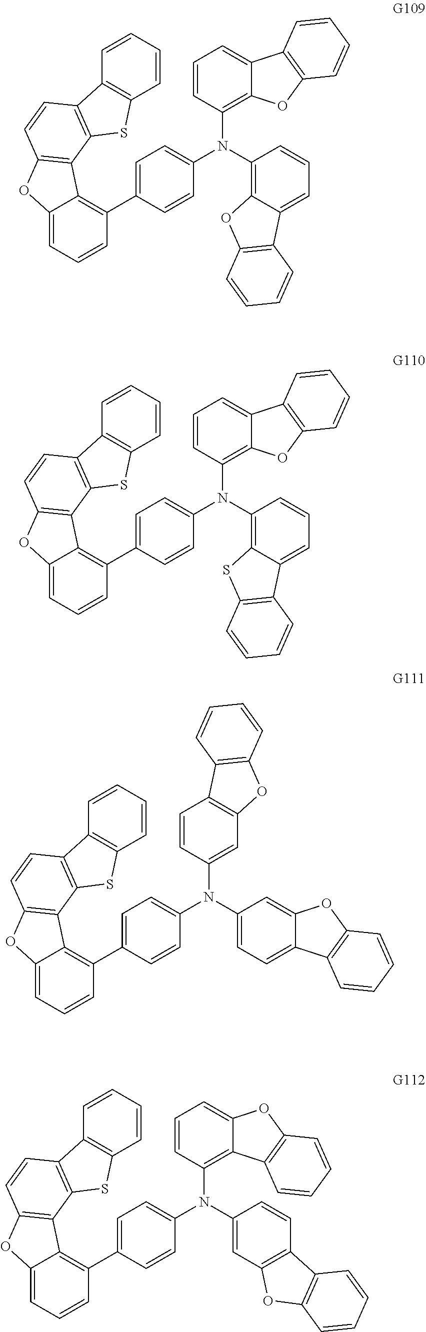

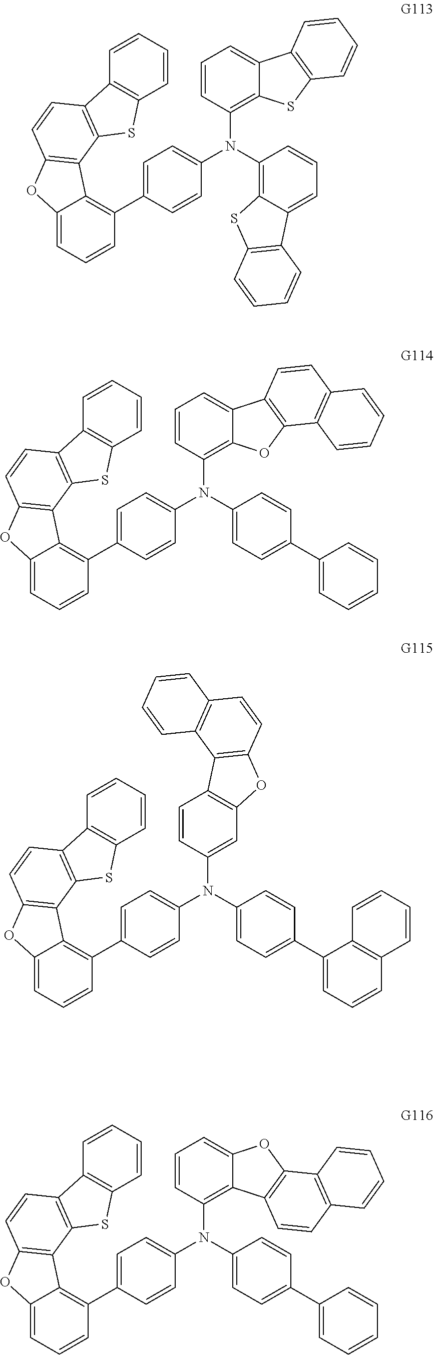 [SMILES: c1cc(-c2ccc(N(c3cccc4c3sc3ccccc34)c3cccc4c3sc3ccccc34)cc2)c2c(c1)oc1ccc3c4ccccc4sc3c12 ; c1ccc(-c2ccc(N(c3ccc(-c4cccc5oc6ccc7c8ccccc8sc7c6c45)cc3)c3cccc4c3oc3c5ccccc5ccc43)cc2)cc1 ; c1ccc2c(-c3ccc(N(c4ccc(-c5cccc6oc7ccc8c9ccccc9sc8c7c56)cc4)c4ccc5c(c4)oc4ccc6ccccc6c45)cc3)cccc2c1 ; c1ccc(-c2ccc(N(c3ccc(-c4cccc5oc6ccc7c8ccccc8sc7c6c45)cc3)c3cccc4oc5c6ccccc6ccc5c34)cc2)cc1]

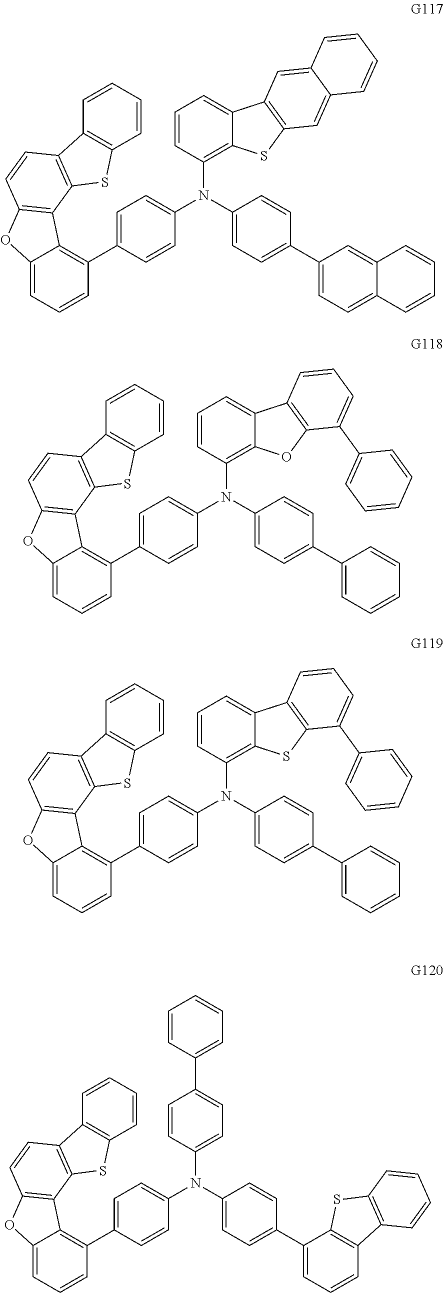 [SMILES: c1ccc2cc(-c3ccc(N(c4ccc(-c5cccc6oc7ccc8c9ccccc9sc8c7c56)cc4)c4cccc5c4sc4cc6ccccc6cc45)cc3)ccc2c1 ; c1ccc(-c2ccc(N(c3ccc(-c4cccc5oc6ccc7c8ccccc8sc7c6c45)cc3)c3cccc4c3oc3c(-c5ccccc5)cccc34)cc2)cc1 ; c1ccc(-c2ccc(N(c3ccc(-c4cccc5oc6ccc7c8ccccc8sc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5oc6ccc7c8ccccc8sc7c6c45)cc3)cc2)cc1]

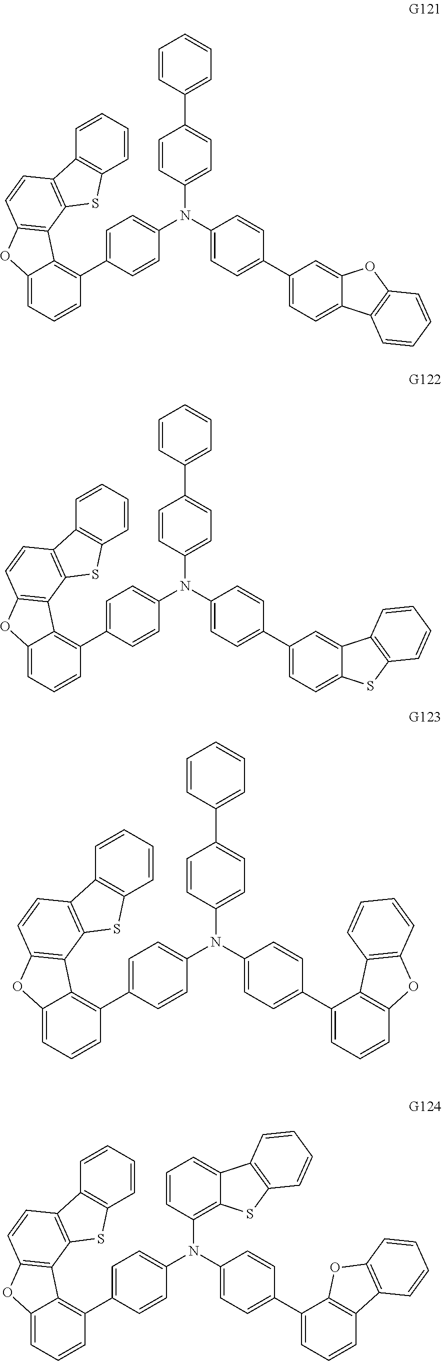 [SMILES: c1ccc(-c2ccc(N(c3ccc(-c4ccc5c(c4)oc4ccccc45)cc3)c3ccc(-c4cccc5oc6ccc7c8ccccc8sc7c6c45)cc3)cc2)cc1 ; c1ccc(-c2ccc(N(c3ccc(-c4ccc5sc6ccccc6c5c4)cc3)c3ccc(-c4cccc5oc6ccc7c8ccccc8sc7c6c45)cc3)cc2)cc1 ; c1ccc(-c2ccc(N(c3ccc(-c4cccc5oc6ccccc6c45)cc3)c3ccc(-c4cccc5oc6ccc7c8ccccc8sc7c6c45)cc3)cc2)cc1 ; c1ccc2c(c1)oc1c(-c3ccc(N(c4ccc(-c5cccc6oc7ccc8c9ccccc9sc8c7c56)cc4)c4cccc5c4sc4ccccc45)cc3)cccc12]

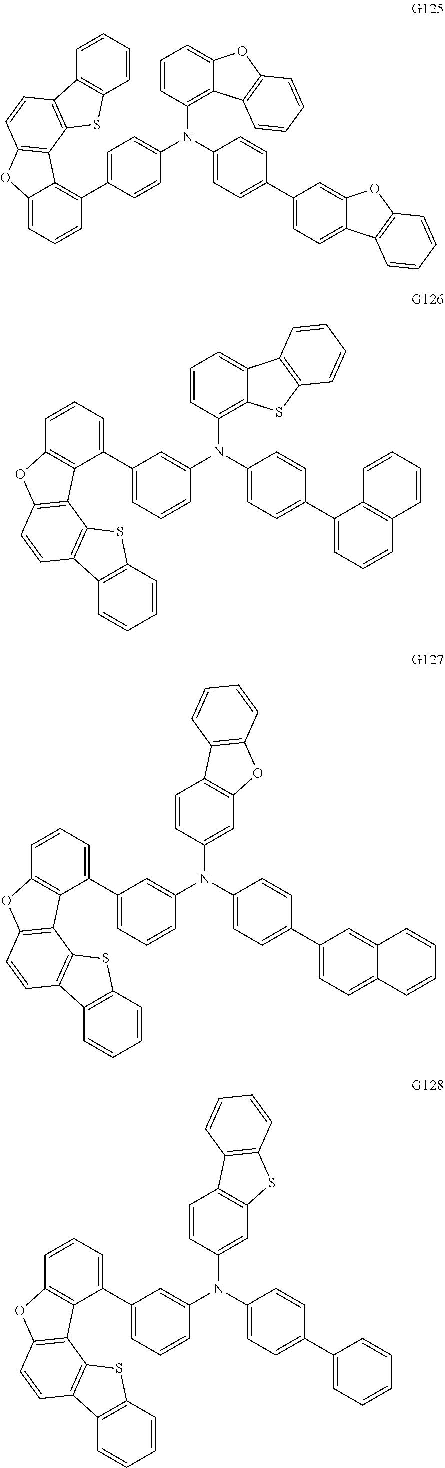 [SMILES: c1ccc2c(c1)oc1cc(-c3ccc(N(c4ccc(-c5cccc6oc7ccc8c9ccccc9sc8c7c56)cc4)c4cccc5oc6ccccc6c45)cc3)ccc12 ; c1cc(-c2cccc3oc4ccc5c6ccccc6sc5c4c23)cc(N(c2ccc(-c3cccc4ccccc34)cc2)c2cccc3c2sc2ccccc23)c1 ; c1cc(-c2cccc3oc4ccc5c6ccccc6sc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5oc6ccc7c8ccccc8sc7c6c45)c3)c3ccc4c(c3)sc3ccccc34)cc2)cc1]

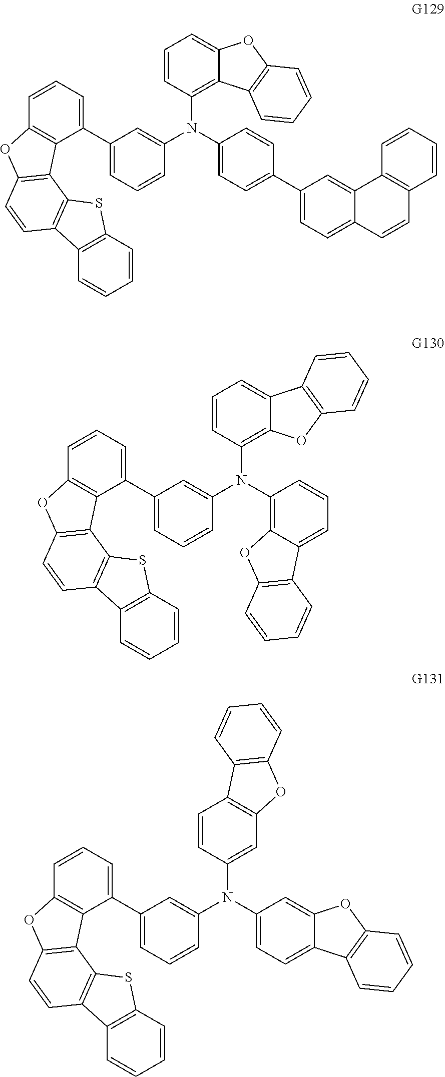

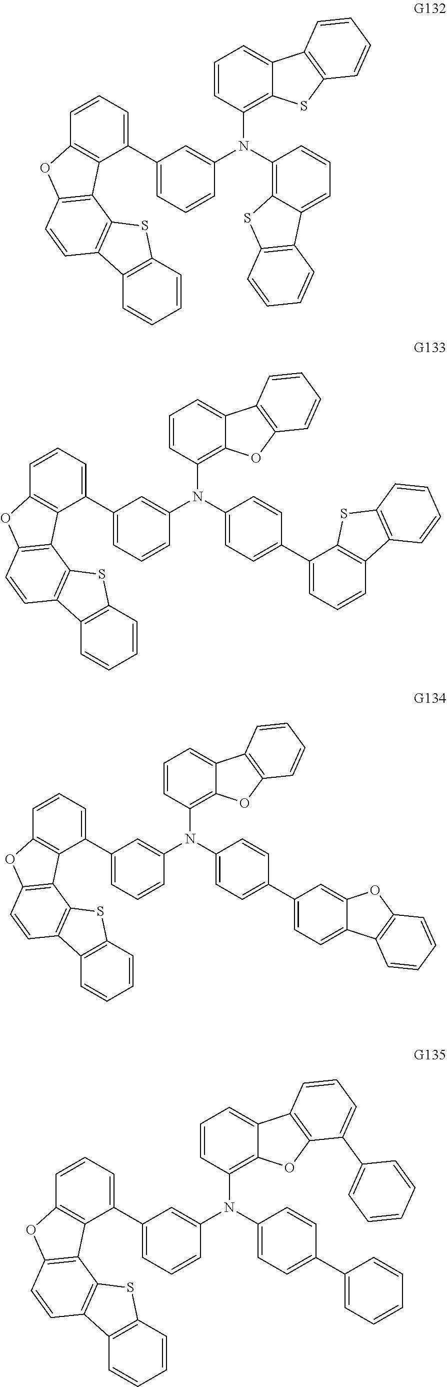

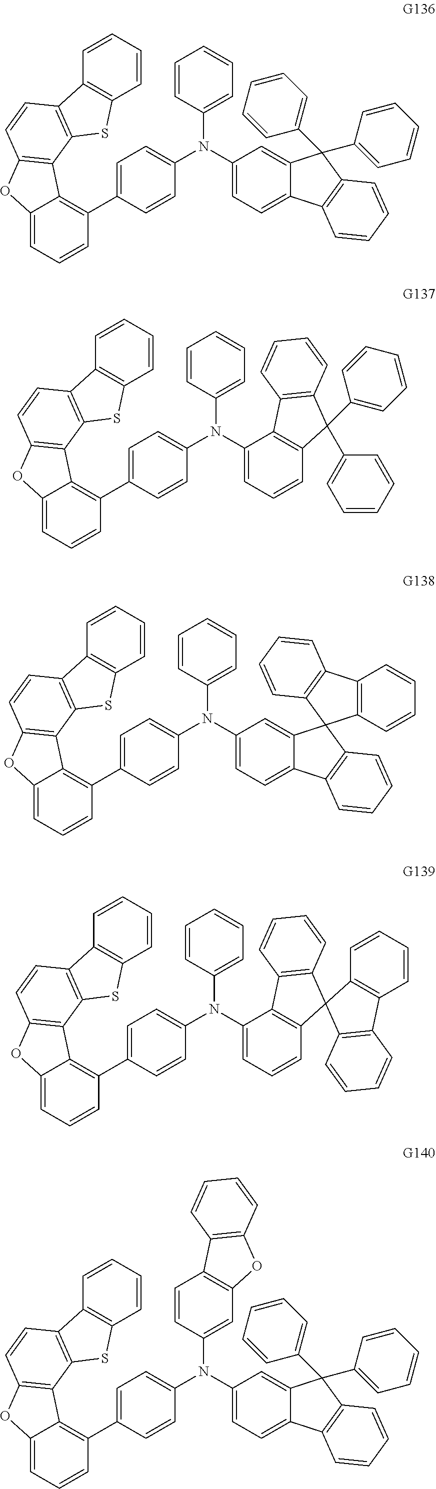

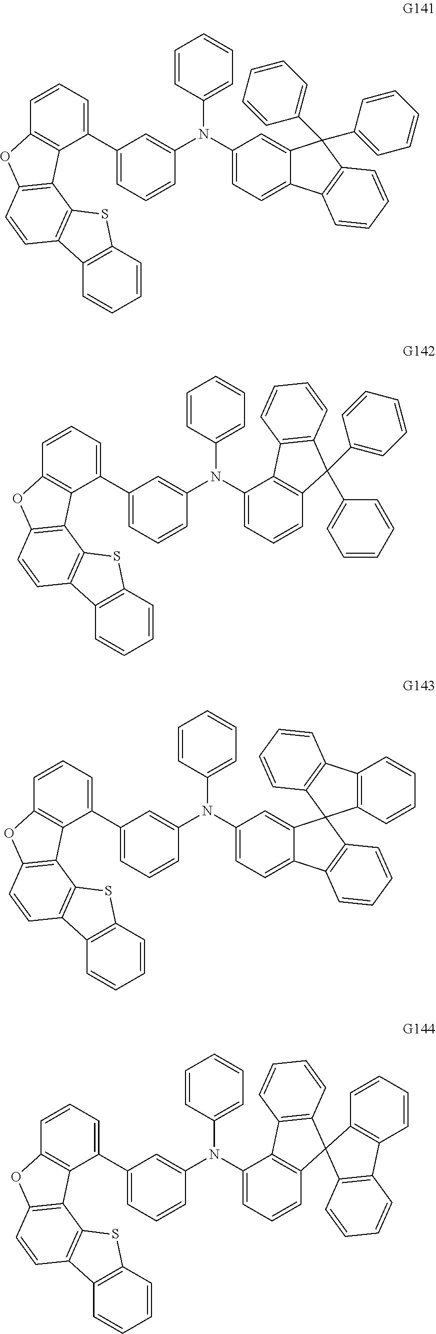

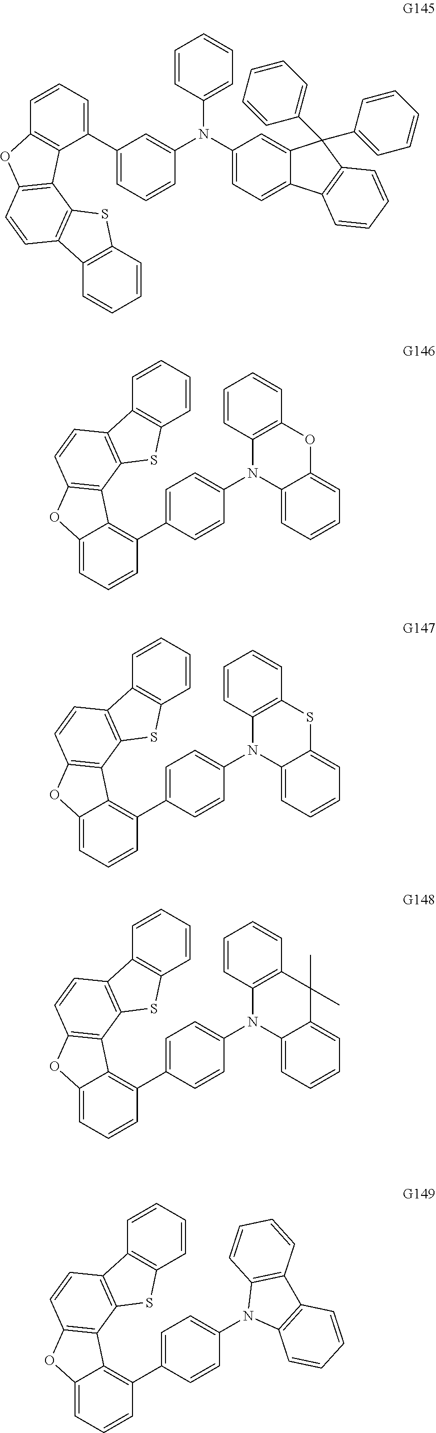

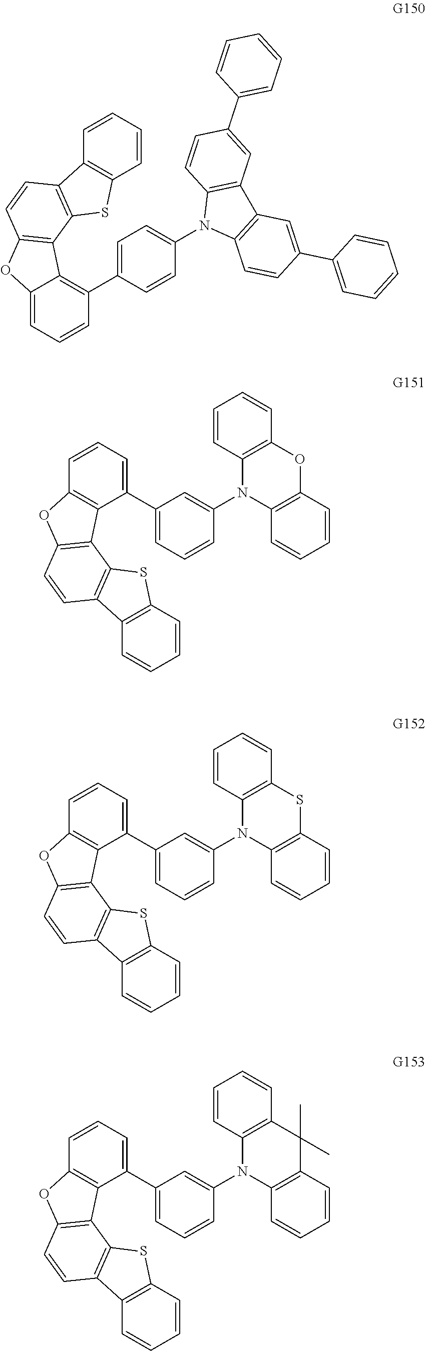 [SMILES: c1ccc(-c2ccc3c(c2)c2cc(-c4ccccc4)ccc2n3-c2ccc(-c3cccc4oc5ccc6c7ccccc7sc6c5c34)cc2)cc1 ; c1cc(-c2cccc3oc4ccc5c6ccccc6sc5c4c23)cc(N2c3ccccc3Oc3ccccc32)c1 ; c1cc(-c2cccc3oc4ccc5c6ccccc6sc5c4c23)cc(N2c3ccccc3Sc3ccccc32)c1 ; CC1(C)c2ccccc2N(c2cccc(-c3cccc4oc5ccc6c7ccccc7sc6c5c34)c2)c2ccccc21]

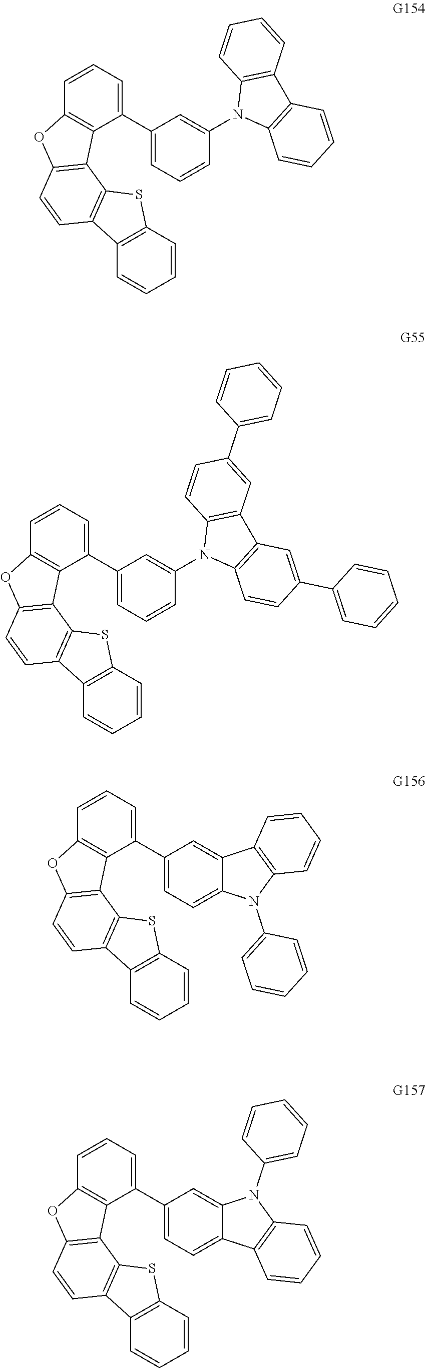 [SMILES: c1cc(-c2cccc3oc4ccc5c6ccccc6sc5c4c23)cc(-n2c3ccccc3c3ccccc32)c1 ; c1ccc(-c2ccc3c(c2)c2cc(-c4ccccc4)ccc2n3-c2cccc(-c3cccc4oc5ccc6c7ccccc7sc6c5c34)c2)cc1 ; c1ccc(-n2c3ccccc3c3cc(-c4cccc5oc6ccc7c8ccccc8sc7c6c45)ccc32)cc1 ; c1ccc(-n2c3ccccc3c3ccc(-c4cccc5oc6ccc7c8ccccc8sc7c6c45)cc32)cc1]

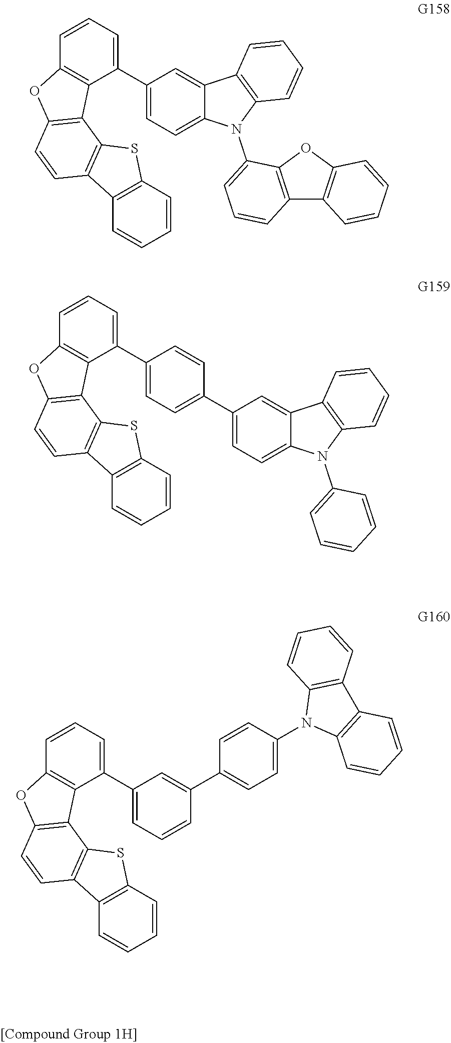 [SMILES: c1ccc2c(c1)oc1c(-n3c4ccccc4c4cc(-c5cccc6oc7ccc8c9ccccc9sc8c7c56)ccc43)cccc12 ; c1ccc(-n2c3ccccc3c3cc(-c4ccc(-c5cccc6oc7ccc8c9ccccc9sc8c7c56)cc4)ccc32)cc1 ; c1cc(-c2ccc(-n3c4ccccc4c4ccccc43)cc2)cc(-c2cccc3oc4ccc5c6ccccc6sc5c4c23)c1]

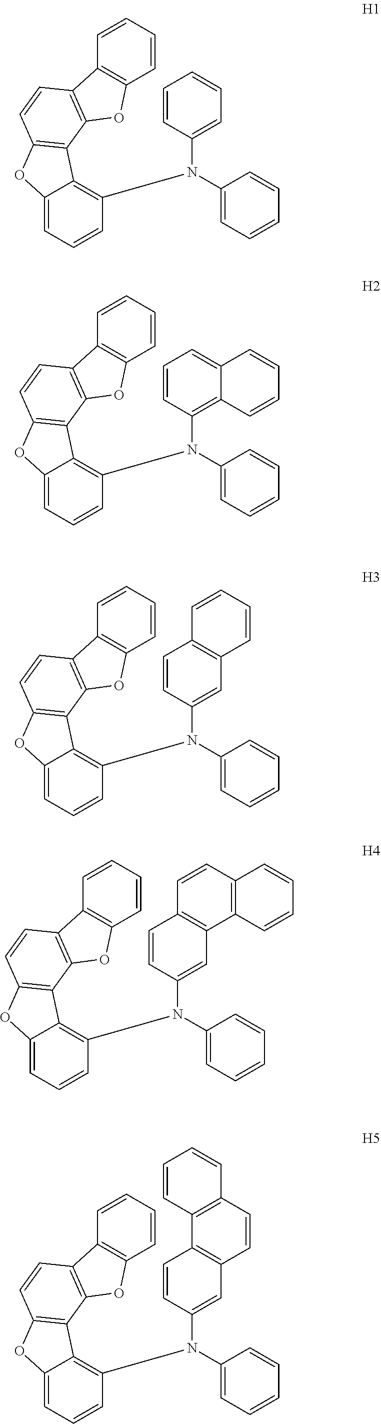 [SMILES: c1ccc(N(c2ccccc2)c2cccc3oc4ccc5c6ccccc6oc5c4c23)cc1 ; c1ccc(N(c2cccc3ccccc23)c2cccc3oc4ccc5c6ccccc6oc5c4c23)cc1 ; c1ccc(N(c2ccc3ccccc3c2)c2cccc3oc4ccc5c6ccccc6oc5c4c23)cc1 ; c1ccc(N(c2ccc3ccc4ccccc4c3c2)c2cccc3oc4ccc5c6ccccc6oc5c4c23)cc1 ; c1ccc(N(c2ccc3c(ccc4ccccc43)c2)c2cccc3oc4ccc5c6ccccc6oc5c4c23)cc1]

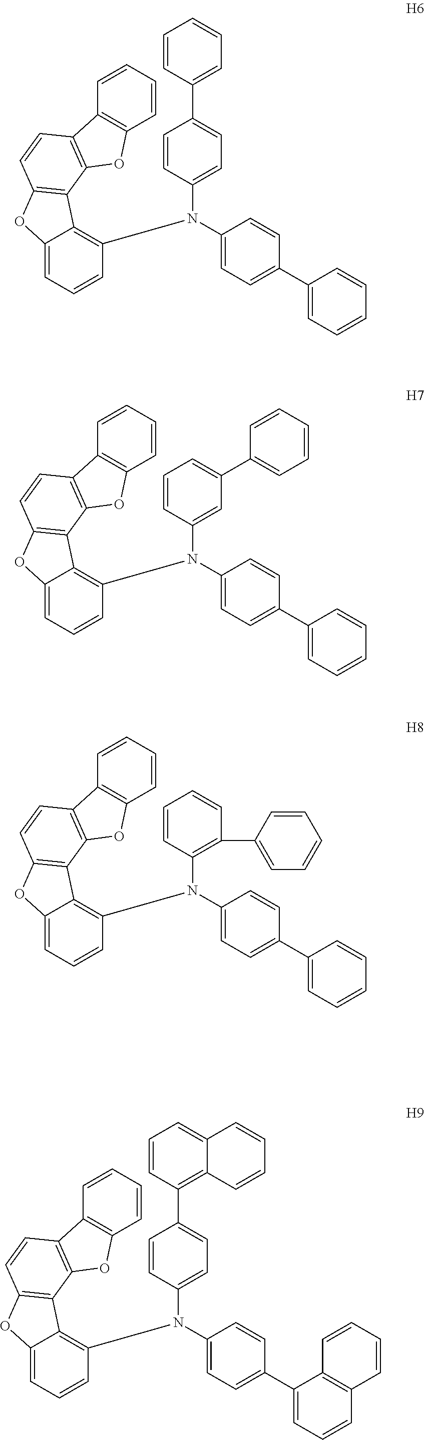 [SMILES: c1ccc(-c2ccc(N(c3ccc(-c4ccccc4)cc3)c3cccc4oc5ccc6c7ccccc7oc6c5c34)cc2)cc1 ; c1ccc(-c2ccc(N(c3cccc(-c4ccccc4)c3)c3cccc4oc5ccc6c7ccccc7oc6c5c34)cc2)cc1 ; c1ccc(-c2ccc(N(c3ccccc3-c3ccccc3)c3cccc4oc5ccc6c7ccccc7oc6c5c34)cc2)cc1 ; c1ccc2c(-c3ccc(N(c4ccc(-c5cccc6ccccc56)cc4)c4cccc5oc6ccc7c8ccccc8oc7c6c45)cc3)cccc2c1]

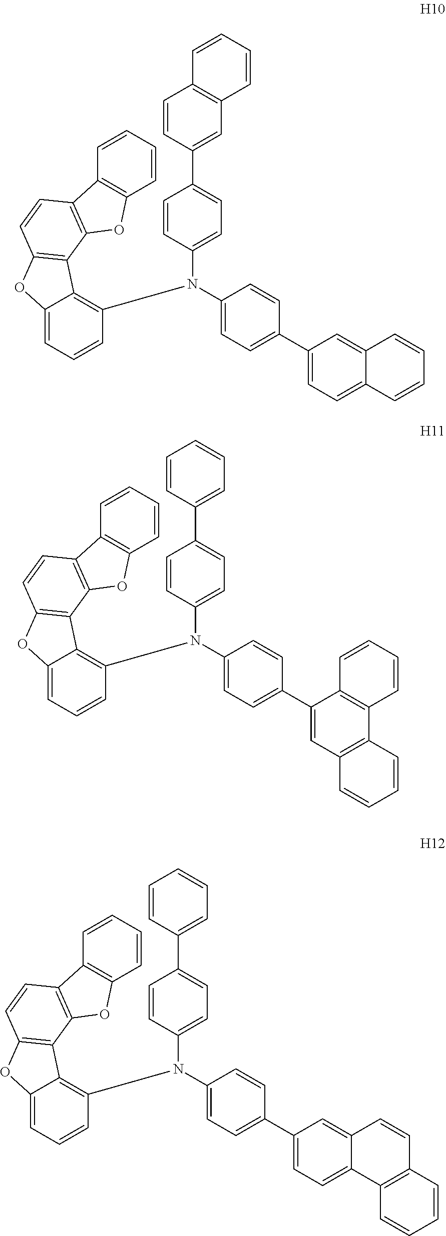 [SMILES: c1ccc2cc(-c3ccc(N(c4ccc(-c5ccc6ccccc6c5)cc4)c4cccc5oc6ccc7c8ccccc8oc7c6c45)cc3)ccc2c1 ; c1ccc(-c2ccc(N(c3ccc(-c4cc5ccccc5c5ccccc45)cc3)c3cccc4oc5ccc6c7ccccc7oc6c5c34)cc2)cc1 ; c1ccc(-c2ccc(N(c3ccc(-c4ccc5c(ccc6ccccc65)c4)cc3)c3cccc4oc5ccc6c7ccccc7oc6c5c34)cc2)cc1]

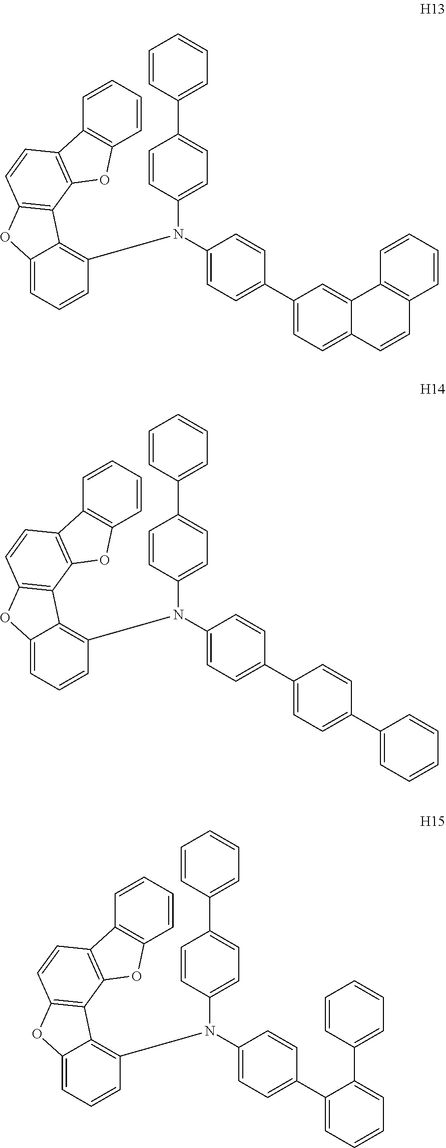 [SMILES: c1ccc(-c2ccc(N(c3ccc(-c4ccc5ccc6ccccc6c5c4)cc3)c3cccc4oc5ccc6c7ccccc7oc6c5c34)cc2)cc1 ; c1ccc(-c2ccc(-c3ccc(N(c4ccc(-c5ccccc5)cc4)c4cccc5oc6ccc7c8ccccc8oc7c6c45)cc3)cc2)cc1 ; c1ccc(-c2ccc(N(c3ccc(-c4ccccc4-c4ccccc4)cc3)c3cccc4oc5ccc6c7ccccc7oc6c5c34)cc2)cc1]

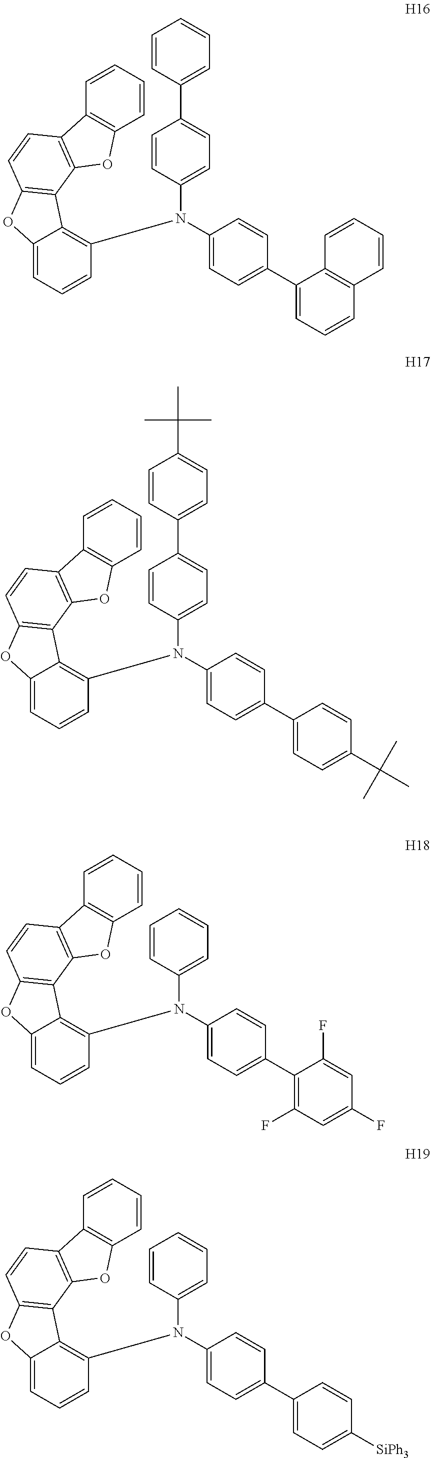

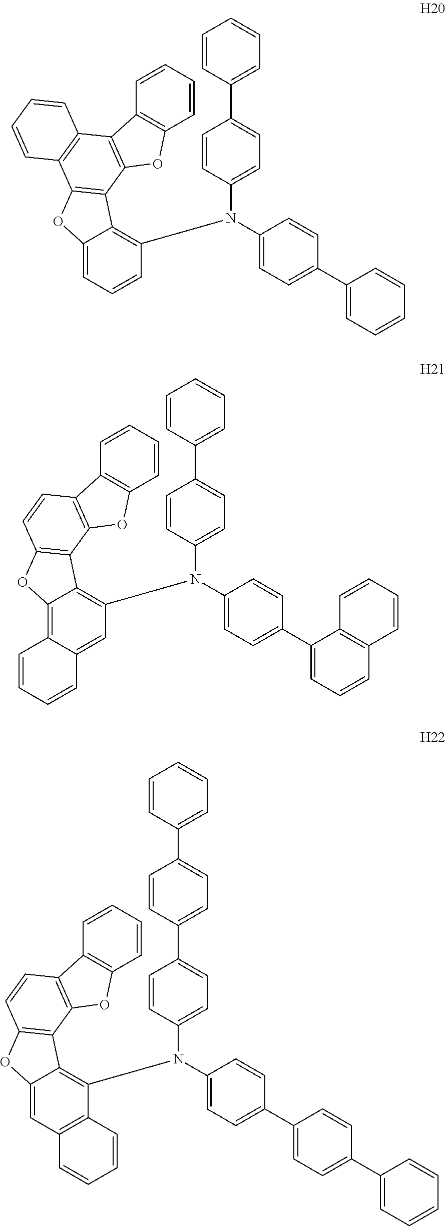 [SMILES: c1ccc(-c2ccc(N(c3ccc(-c4ccccc4)cc3)c3cccc4oc5c6ccccc6c6c7ccccc7oc6c5c34)cc2)cc1 ; c1ccc(-c2ccc(N(c3ccc(-c4cccc5ccccc45)cc3)c3cc4ccccc4c4oc5ccc6c7ccccc7oc6c5c34)cc2)cc1 ; c1ccc(-c2ccc(-c3ccc(N(c4ccc(-c5ccc(-c6ccccc6)cc5)cc4)c4c5ccccc5cc5oc6ccc7c8ccccc8oc7c6c45)cc3)cc2)cc1]

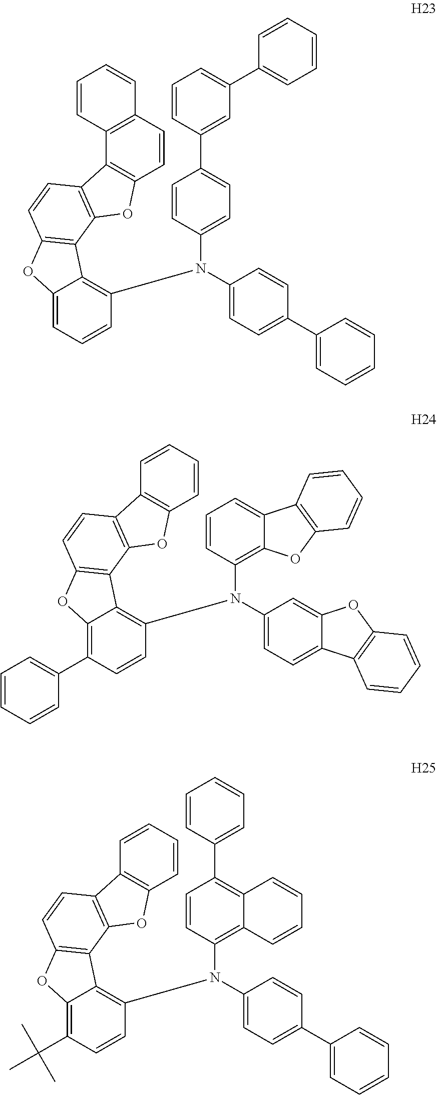 [SMILES: c1ccc(-c2ccc(N(c3ccc(-c4cccc(-c5ccccc5)c4)cc3)c3cccc4oc5ccc6c(oc7ccc8ccccc8c76)c5c34)cc2)cc1 ; c1ccc(-c2ccc(N(c3ccc4c(c3)oc3ccccc34)c3cccc4c3oc3ccccc34)c3c2oc2ccc4c5ccccc5oc4c23)cc1 ; CC(C)(C)c1ccc(N(c2ccc(-c3ccccc3)cc2)c2ccc(-c3ccccc3)c3ccccc23)c2c1oc1ccc3c4ccccc4oc3c12]

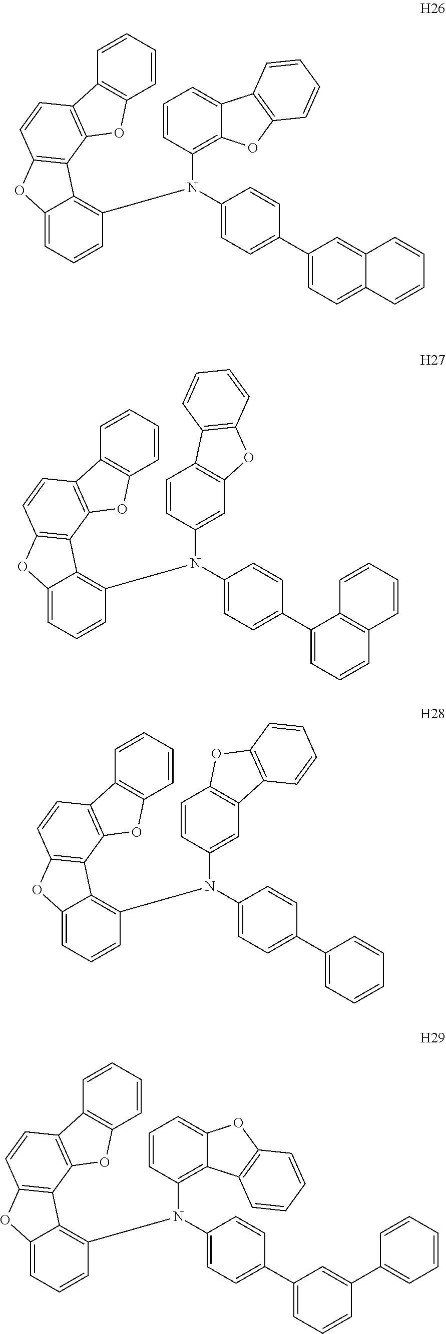 [SMILES: c1ccc2cc(-c3ccc(N(c4cccc5c4oc4ccccc45)c4cccc5oc6ccc7c8ccccc8oc7c6c45)cc3)ccc2c1 ; c1ccc2c(-c3ccc(N(c4ccc5c(c4)oc4ccccc45)c4cccc5oc6ccc7c8ccccc8oc7c6c45)cc3)cccc2c1 ; c1ccc(-c2ccc(N(c3ccc4oc5ccccc5c4c3)c3cccc4oc5ccc6c7ccccc7oc6c5c34)cc2)cc1 ; c1ccc(-c2cccc(-c3ccc(N(c4cccc5oc6ccccc6c45)c4cccc5oc6ccc7c8ccccc8oc7c6c45)cc3)c2)cc1]

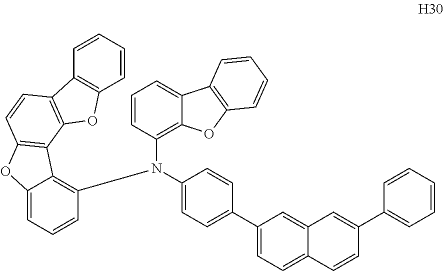 [SMILES: c1ccc(-c2ccc3ccc(-c4ccc(N(c5cccc6c5oc5ccccc56)c5cccc6oc7ccc8c9ccccc9oc8c7c56)cc4)cc3c2)cc1]

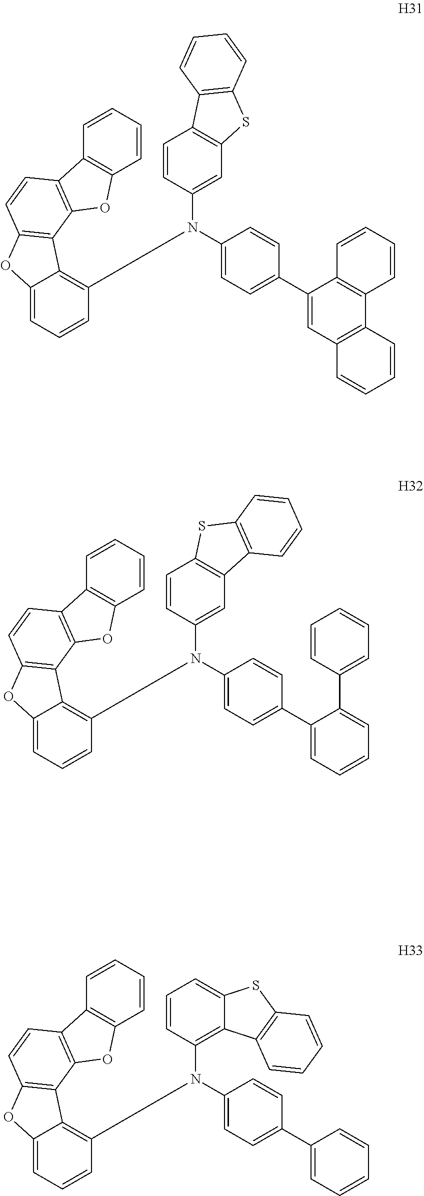 [SMILES: c1ccc2c(c1)cc(-c1ccc(N(c3ccc4c(c3)sc3ccccc34)c3cccc4oc5ccc6c7ccccc7oc6c5c34)cc1)c1ccccc12 ; c1ccc(-c2ccccc2-c2ccc(N(c3ccc4sc5ccccc5c4c3)c3cccc4oc5ccc6c7ccccc7oc6c5c34)cc2)cc1 ; c1ccc(-c2ccc(N(c3cccc4sc5ccccc5c34)c3cccc4oc5ccc6c7ccccc7oc6c5c34)cc2)cc1]

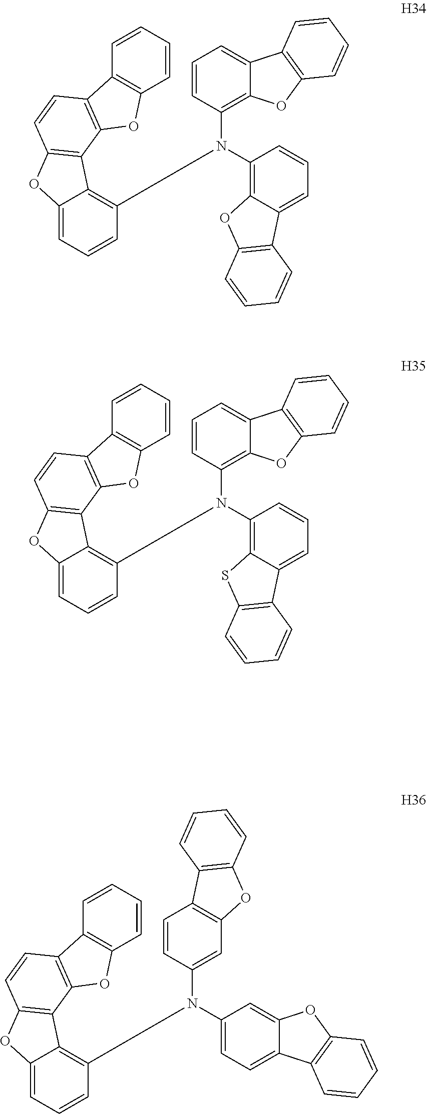 [SMILES: c1ccc2c(c1)oc1c(N(c3cccc4c3oc3ccccc34)c3cccc4oc5ccc6c7ccccc7oc6c5c34)cccc12 ; c1ccc2c(c1)oc1c(N(c3cccc4c3sc3ccccc34)c3cccc4oc5ccc6c7ccccc7oc6c5c34)cccc12 ; c1ccc2c(c1)oc1cc(N(c3ccc4c(c3)oc3ccccc34)c3cccc4oc5ccc6c7ccccc7oc6c5c34)ccc12]

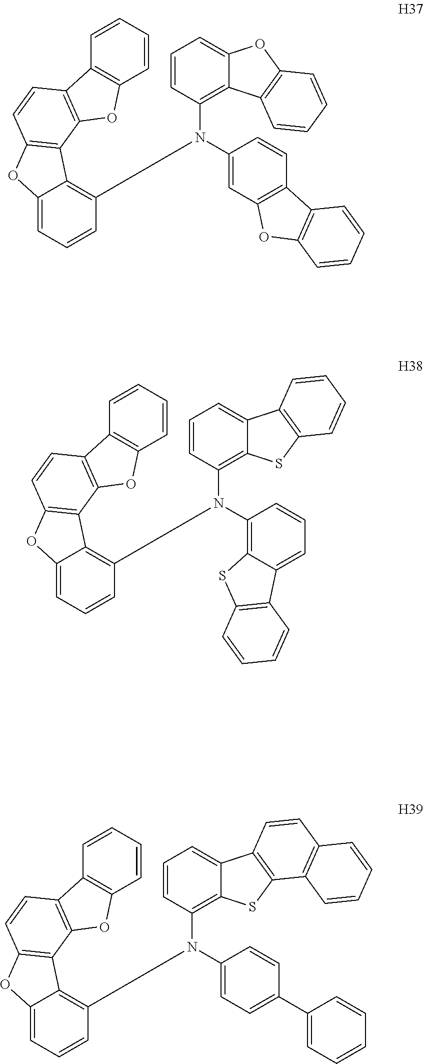 [SMILES: c1ccc2c(c1)oc1cc(N(c3cccc4oc5ccccc5c34)c3cccc4oc5ccc6c7ccccc7oc6c5c34)ccc12 ; c1ccc2c(c1)oc1c2ccc2oc3cccc(N(c4cccc5c4sc4ccccc45)c4cccc5c4sc4ccccc45)c3c21 ; c1ccc(-c2ccc(N(c3cccc4c3sc3c5ccccc5ccc43)c3cccc4oc5ccc6c7ccccc7oc6c5c34)cc2)cc1]

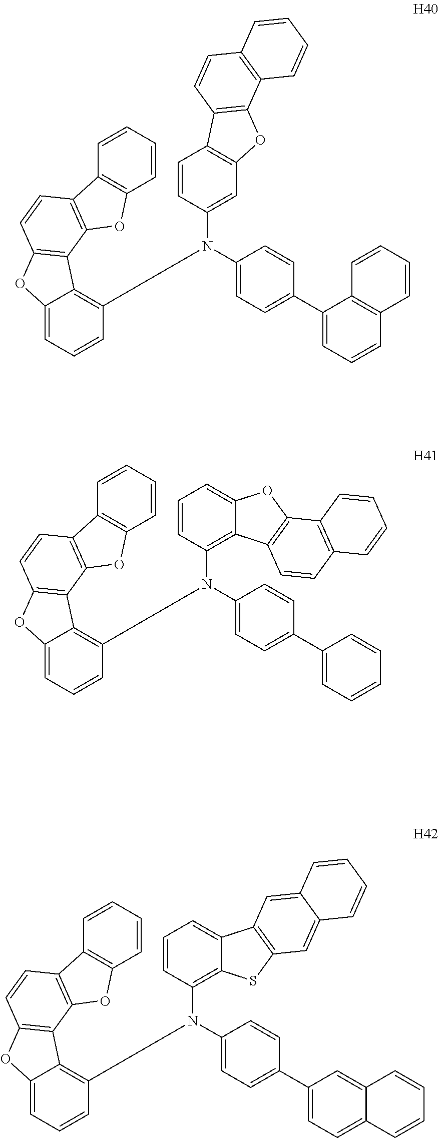 [SMILES: c1ccc2c(-c3ccc(N(c4ccc5c(c4)oc4c6ccccc6ccc54)c4cccc5oc6ccc7c8ccccc8oc7c6c45)cc3)cccc2c1 ; c1ccc(-c2ccc(N(c3cccc4oc5c6ccccc6ccc5c34)c3cccc4oc5ccc6c7ccccc7oc6c5c34)cc2)cc1 ; c1ccc2cc(-c3ccc(N(c4cccc5c4sc4cc6ccccc6cc45)c4cccc5oc6ccc7c8ccccc8oc7c6c45)cc3)ccc2c1]

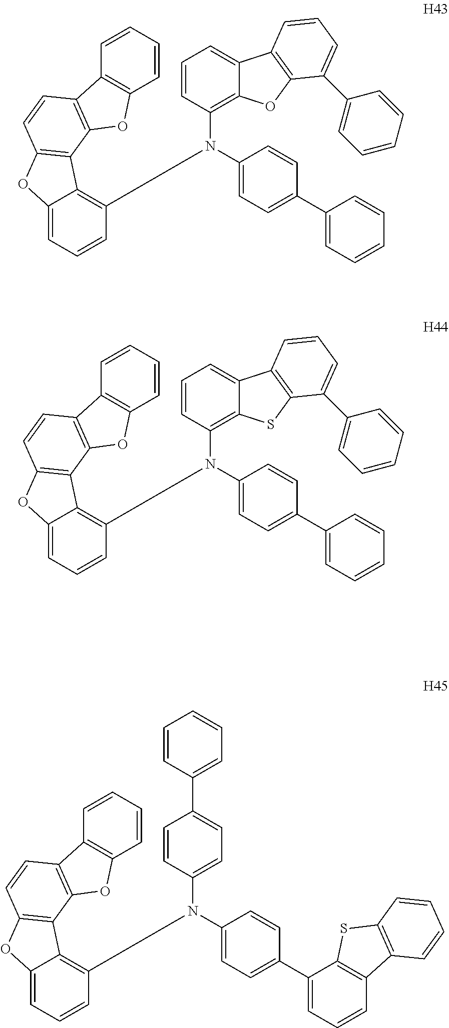 [SMILES: c1ccc(-c2ccc(N(c3cccc4c3oc3c(-c5ccccc5)cccc34)c3cccc4oc5ccc6c7ccccc7oc6c5c34)cc2)cc1 ; c1ccc(-c2ccc(N(c3cccc4c3sc3c(-c5ccccc5)cccc34)c3cccc4oc5ccc6c7ccccc7oc6c5c34)cc2)cc1 ; c1ccc(-c2ccc(N(c3ccc(-c4cccc5c4sc4ccccc45)cc3)c3cccc4oc5ccc6c7ccccc7oc6c5c34)cc2)cc1]

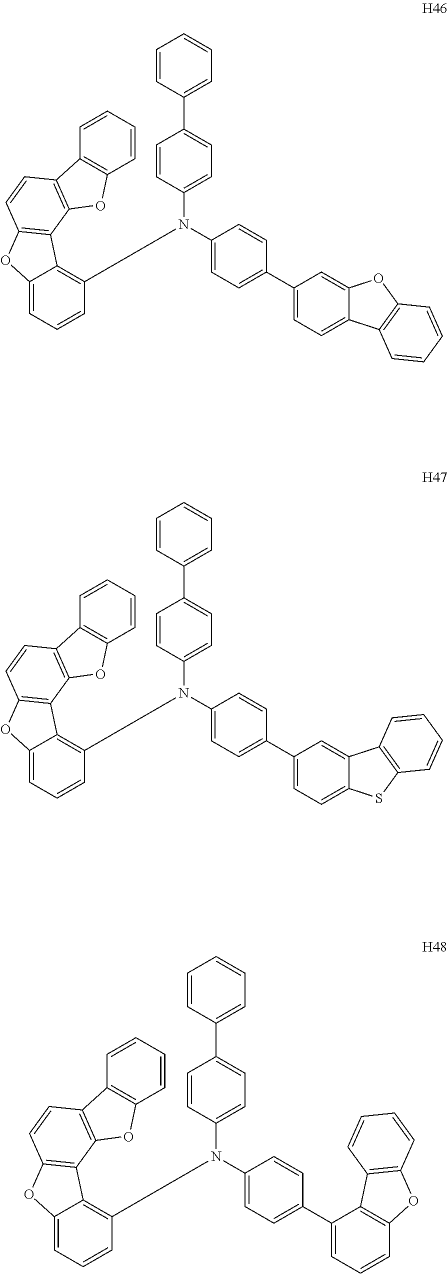 [SMILES: c1ccc(-c2ccc(N(c3ccc(-c4ccc5c(c4)oc4ccccc45)cc3)c3cccc4oc5ccc6c7ccccc7oc6c5c34)cc2)cc1 ; c1ccc(-c2ccc(N(c3ccc(-c4ccc5sc6ccccc6c5c4)cc3)c3cccc4oc5ccc6c7ccccc7oc6c5c34)cc2)cc1 ; c1ccc(-c2ccc(N(c3ccc(-c4cccc5oc6ccccc6c45)cc3)c3cccc4oc5ccc6c7ccccc7oc6c5c34)cc2)cc1]

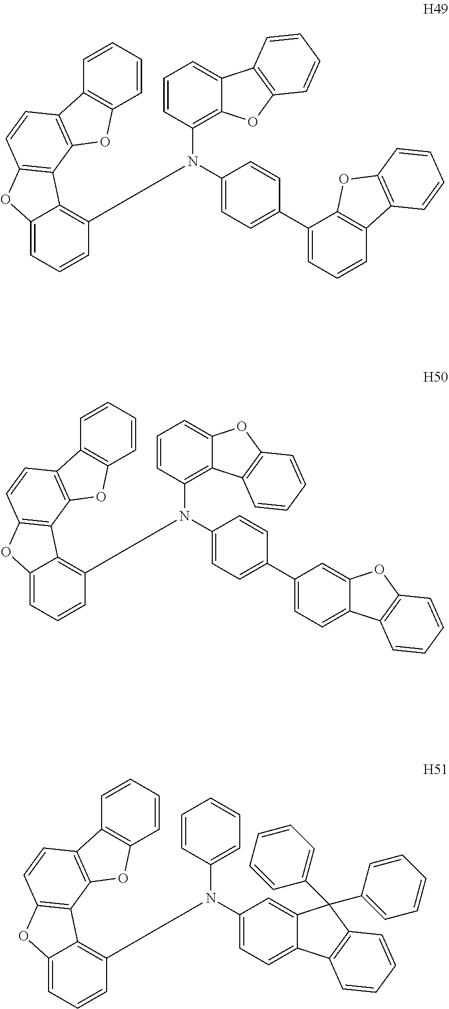 [SMILES: c1ccc2c(c1)oc1c(-c3ccc(N(c4cccc5c4oc4ccccc45)c4cccc5oc6ccc7c8ccccc8oc7c6c45)cc3)cccc12 ; c1ccc2c(c1)oc1cc(-c3ccc(N(c4cccc5oc6ccccc6c45)c4cccc5oc6ccc7c8ccccc8oc7c6c45)cc3)ccc12 ; c1ccc(N(c2ccc3c(c2)C(c2ccccc2)(c2ccccc2)c2ccccc2-3)c2cccc3oc4ccc5c6ccccc6oc5c4c23)cc1]

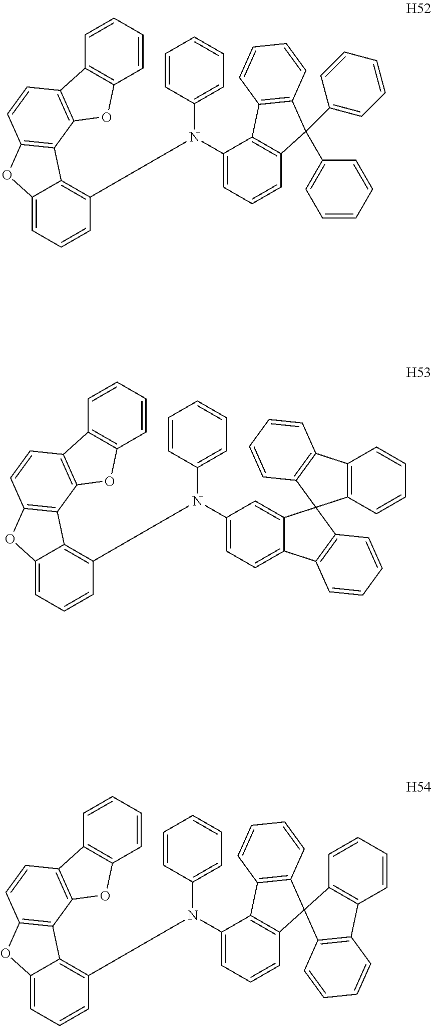 [SMILES: c1ccc(N(c2cccc3c2-c2ccccc2C3(c2ccccc2)c2ccccc2)c2cccc3oc4ccc5c6ccccc6oc5c4c23)cc1 ; c1ccc(N(c2ccc3c(c2)C2(c4ccccc4-c4ccccc42)c2ccccc2-3)c2cccc3oc4ccc5c6ccccc6oc5c4c23)cc1 ; c1ccc(N(c2cccc3c2-c2ccccc2C32c3ccccc3-c3ccccc32)c2cccc3oc4ccc5c6ccccc6oc5c4c23)cc1]

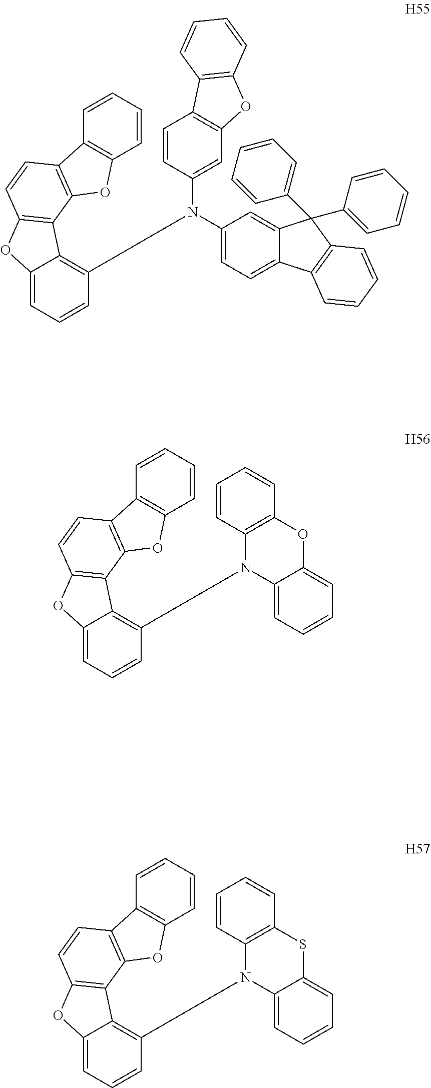 [SMILES: c1ccc(C2(c3ccccc3)c3ccccc3-c3ccc(N(c4ccc5c(c4)oc4ccccc45)c4cccc5oc6ccc7c8ccccc8oc7c6c45)cc32)cc1 ; c1ccc2c(c1)Oc1ccccc1N2c1cccc2oc3ccc4c5ccccc5oc4c3c12 ; c1ccc2c(c1)Sc1ccccc1N2c1cccc2oc3ccc4c5ccccc5oc4c3c12]

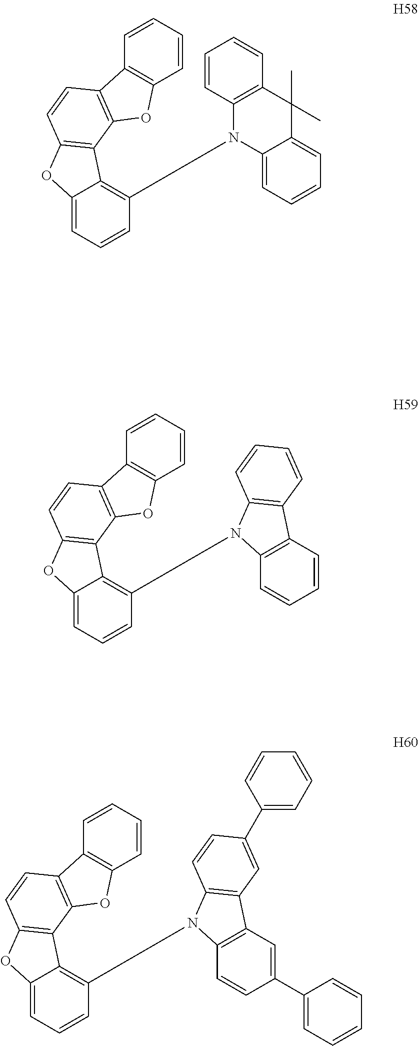 [SMILES: CC1(C)c2ccccc2N(c2cccc3oc4ccc5c6ccccc6oc5c4c23)c2ccccc21 ; c1ccc2c(c1)oc1c2ccc2oc3cccc(-n4c5ccccc5c5ccccc54)c3c21 ; c1ccc(-c2ccc3c(c2)c2cc(-c4ccccc4)ccc2n3-c2cccc3oc4ccc5c6ccccc6oc5c4c23)cc1]

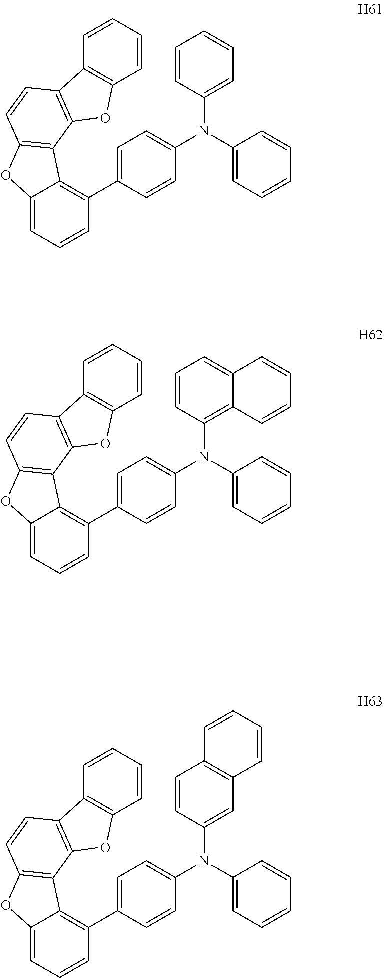 [SMILES: c1ccc(N(c2ccccc2)c2ccc(-c3cccc4oc5ccc6c7ccccc7oc6c5c34)cc2)cc1 ; c1ccc(N(c2ccc(-c3cccc4oc5ccc6c7ccccc7oc6c5c34)cc2)c2cccc3ccccc23)cc1 ; c1ccc(N(c2ccc(-c3cccc4oc5ccc6c7ccccc7oc6c5c34)cc2)c2ccc3ccccc3c2)cc1]

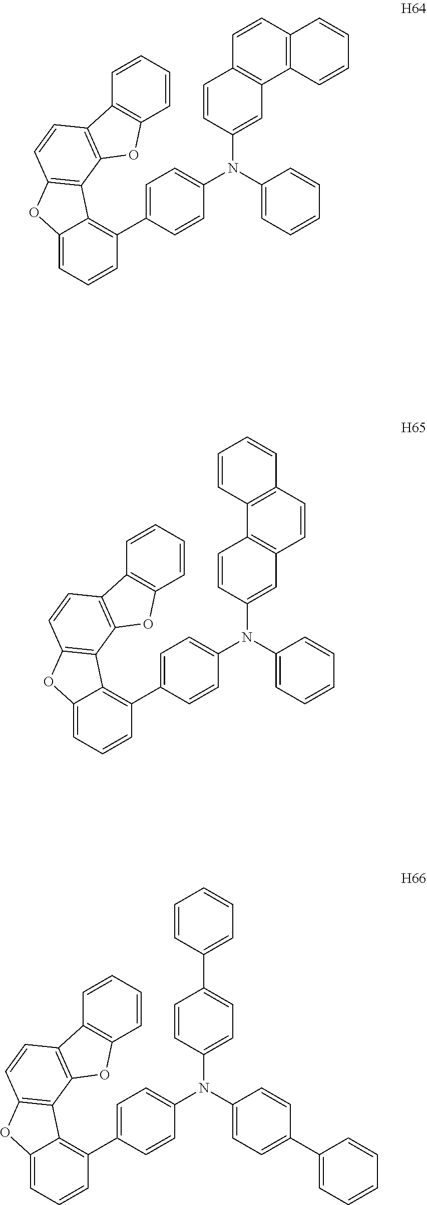 [SMILES: c1ccc(N(c2ccc(-c3cccc4oc5ccc6c7ccccc7oc6c5c34)cc2)c2ccc3ccc4ccccc4c3c2)cc1 ; c1ccc(N(c2ccc(-c3cccc4oc5ccc6c7ccccc7oc6c5c34)cc2)c2ccc3c(ccc4ccccc43)c2)cc1 ; c1ccc(-c2ccc(N(c3ccc(-c4ccccc4)cc3)c3ccc(-c4cccc5oc6ccc7c8ccccc8oc7c6c45)cc3)cc2)cc1]

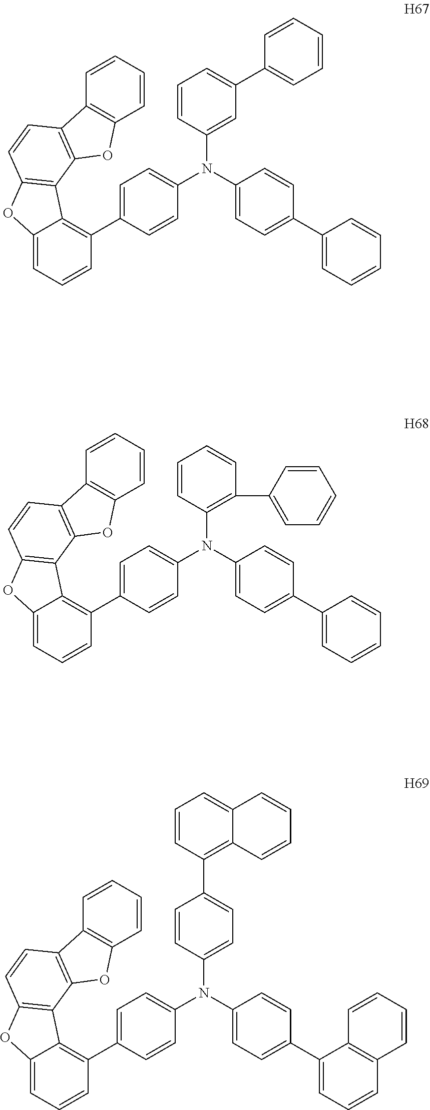 [SMILES: c1ccc(-c2ccc(N(c3ccc(-c4cccc5oc6ccc7c8ccccc8oc7c6c45)cc3)c3cccc(-c4ccccc4)c3)cc2)cc1 ; c1ccc(-c2ccc(N(c3ccc(-c4cccc5oc6ccc7c8ccccc8oc7c6c45)cc3)c3ccccc3-c3ccccc3)cc2)cc1 ; c1ccc2c(-c3ccc(N(c4ccc(-c5cccc6ccccc56)cc4)c4ccc(-c5cccc6oc7ccc8c9ccccc9oc8c7c56)cc4)cc3)cccc2c1]

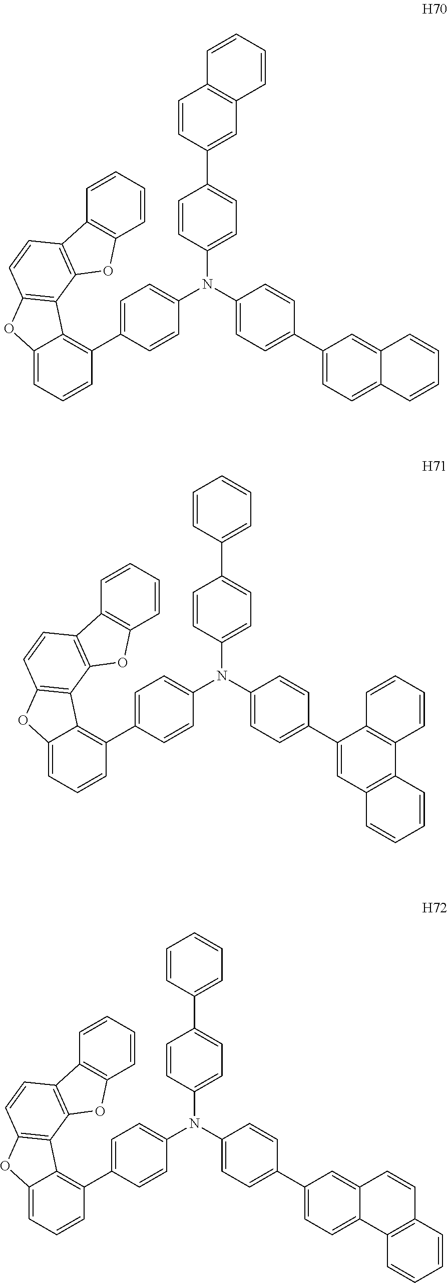 [SMILES: c1ccc2cc(-c3ccc(N(c4ccc(-c5ccc6ccccc6c5)cc4)c4ccc(-c5cccc6oc7ccc8c9ccccc9oc8c7c56)cc4)cc3)ccc2c1 ; c1ccc(-c2ccc(N(c3ccc(-c4cc5ccccc5c5ccccc45)cc3)c3ccc(-c4cccc5oc6ccc7c8ccccc8oc7c6c45)cc3)cc2)cc1 ; c1ccc(-c2ccc(N(c3ccc(-c4ccc5c(ccc6ccccc65)c4)cc3)c3ccc(-c4cccc5oc6ccc7c8ccccc8oc7c6c45)cc3)cc2)cc1]

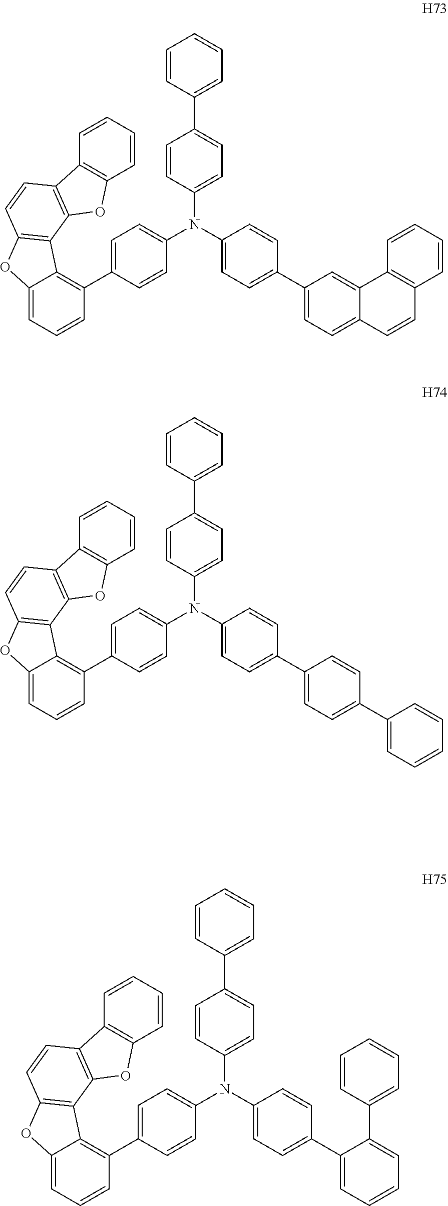 [SMILES: c1ccc(-c2ccc(N(c3ccc(-c4ccc5ccc6ccccc6c5c4)cc3)c3ccc(-c4cccc5oc6ccc7c8ccccc8oc7c6c45)cc3)cc2)cc1 ; c1ccc(-c2ccc(-c3ccc(N(c4ccc(-c5ccccc5)cc4)c4ccc(-c5cccc6oc7ccc8c9ccccc9oc8c7c56)cc4)cc3)cc2)cc1 ; c1ccc(-c2ccc(N(c3ccc(-c4ccccc4-c4ccccc4)cc3)c3ccc(-c4cccc5oc6ccc7c8ccccc8oc7c6c45)cc3)cc2)cc1]

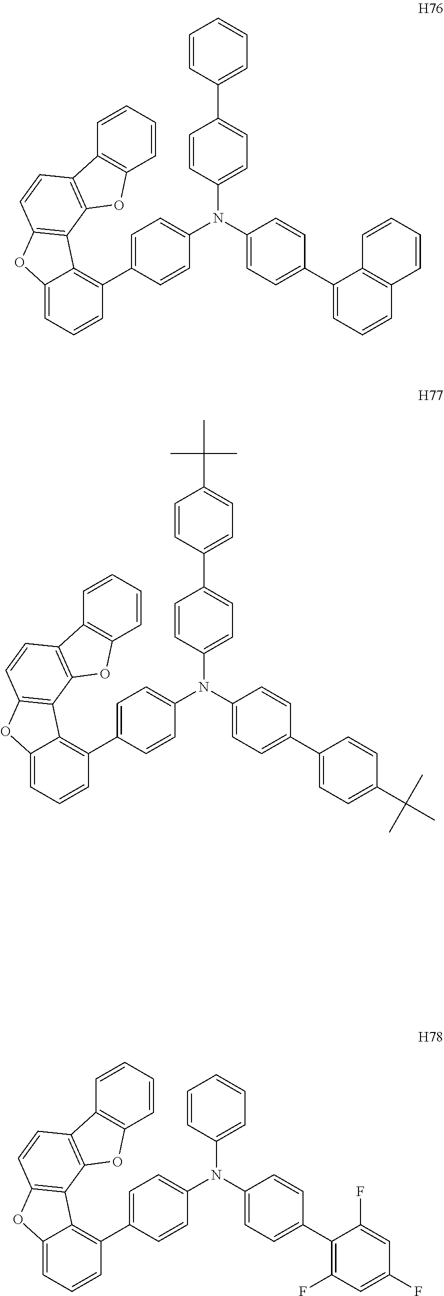 [SMILES: c1ccc(-c2ccc(N(c3ccc(-c4cccc5ccccc45)cc3)c3ccc(-c4cccc5oc6ccc7c8ccccc8oc7c6c45)cc3)cc2)cc1 ; CC(C)(C)c1ccc(-c2ccc(N(c3ccc(-c4ccc(C(C)(C)C)cc4)cc3)c3ccc(-c4cccc5oc6ccc7c8ccccc8oc7c6c45)cc3)cc2)cc1 ; Fc1cc(F)c(-c2ccc(N(c3ccccc3)c3ccc(-c4cccc5oc6ccc7c8ccccc8oc7c6c45)cc3)cc2)c(F)c1]

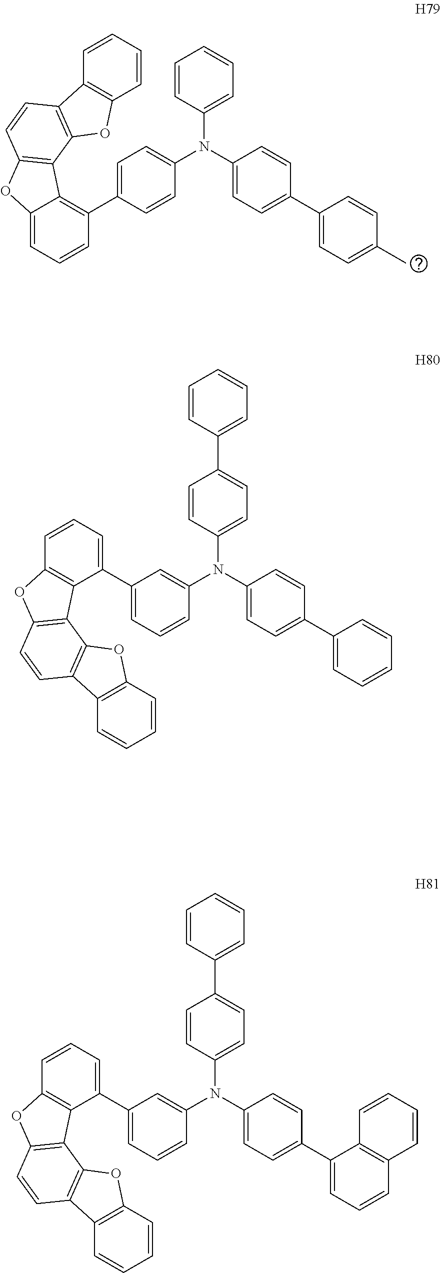 [SMILES: Cc1ccc(-c2ccc(N(c3ccccc3)c3ccc(-c4cccc5oc6ccc7c8ccccc8oc7c6c45)cc3)cc2)cc1 ; c1ccc(-c2ccc(N(c3ccc(-c4ccccc4)cc3)c3cccc(-c4cccc5oc6ccc7c8ccccc8oc7c6c45)c3)cc2)cc1 ; c1ccc(-c2ccc(N(c3ccc(-c4cccc5ccccc45)cc3)c3cccc(-c4cccc5oc6ccc7c8ccccc8oc7c6c45)c3)cc2)cc1]

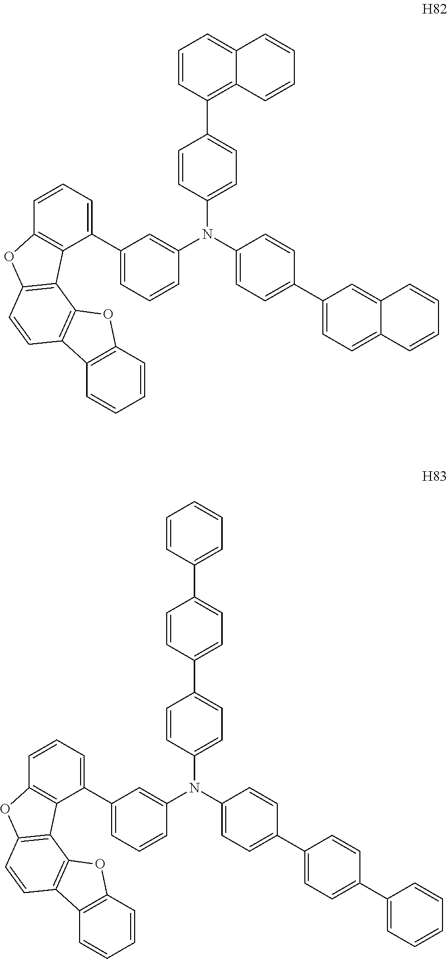 [SMILES: c1cc(-c2cccc3oc4ccc5c6ccccc6oc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6oc7ccc8c9ccccc9oc8c7c56)c4)cc3)cc2)cc1]

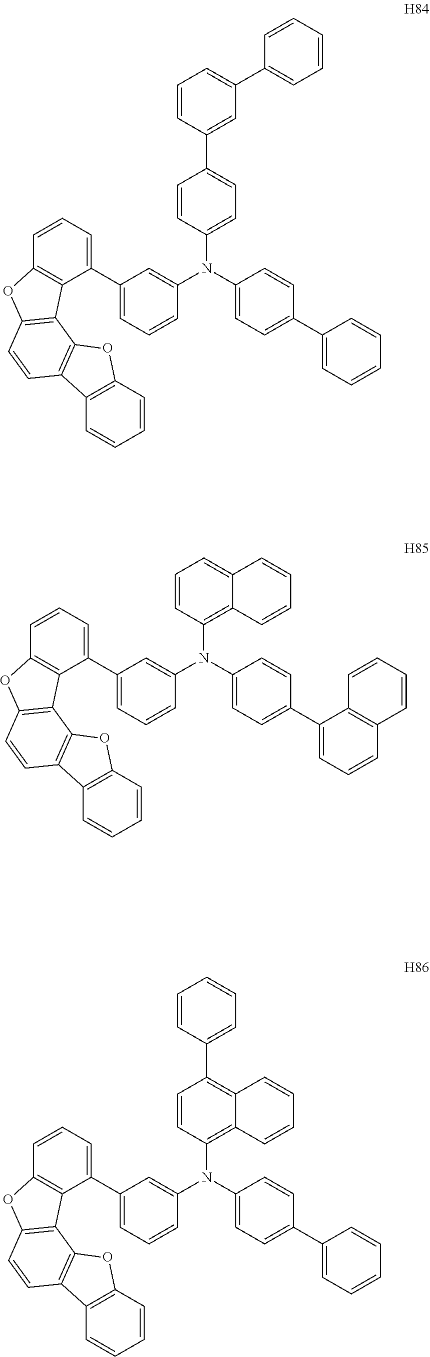 [SMILES: c1ccc(-c2ccc(N(c3ccc(-c4cccc(-c5ccccc5)c4)cc3)c3cccc(-c4cccc5oc6ccc7c8ccccc8oc7c6c45)c3)cc2)cc1 ; c1cc(-c2cccc3oc4ccc5c6ccccc6oc5c4c23)cc(N(c2ccc(-c3cccc4ccccc34)cc2)c2cccc3ccccc23)c1 ; c1ccc(-c2ccc(N(c3cccc(-c4cccc5oc6ccc7c8ccccc8oc7c6c45)c3)c3ccc(-c4ccccc4)c4ccccc34)cc2)cc1]

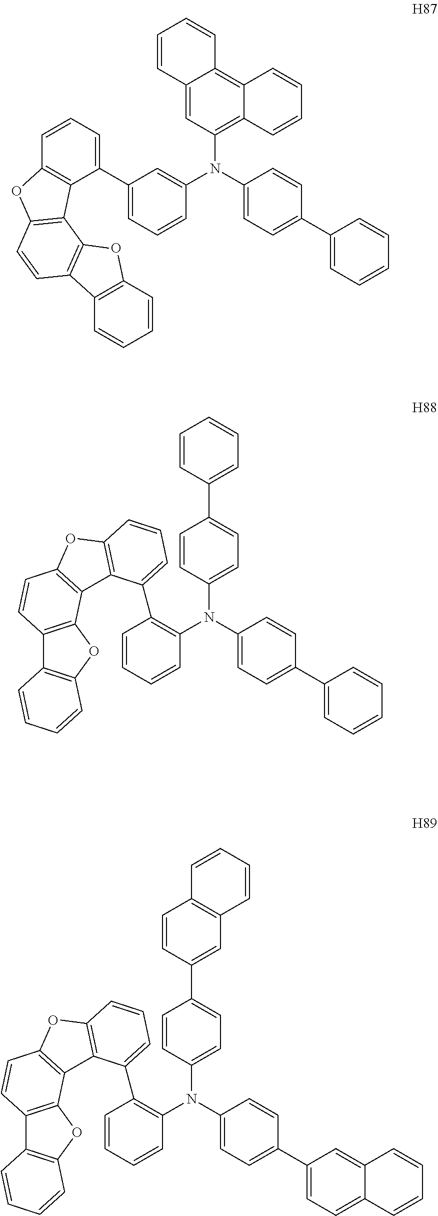 [SMILES: c1ccc(-c2ccc(N(c3cccc(-c4cccc5oc6ccc7c8ccccc8oc7c6c45)c3)c3cc4ccccc4c4ccccc34)cc2)cc1 ; c1ccc(-c2ccc(N(c3ccc(-c4ccccc4)cc3)c3ccccc3-c3cccc4oc5ccc6c7ccccc7oc6c5c34)cc2)cc1 ; c1ccc(N(c2ccc(-c3ccc4ccccc4c3)cc2)c2ccc(-c3ccc4ccccc4c3)cc2)c(-c2cccc3oc4ccc5c6ccccc6oc5c4c23)c1]

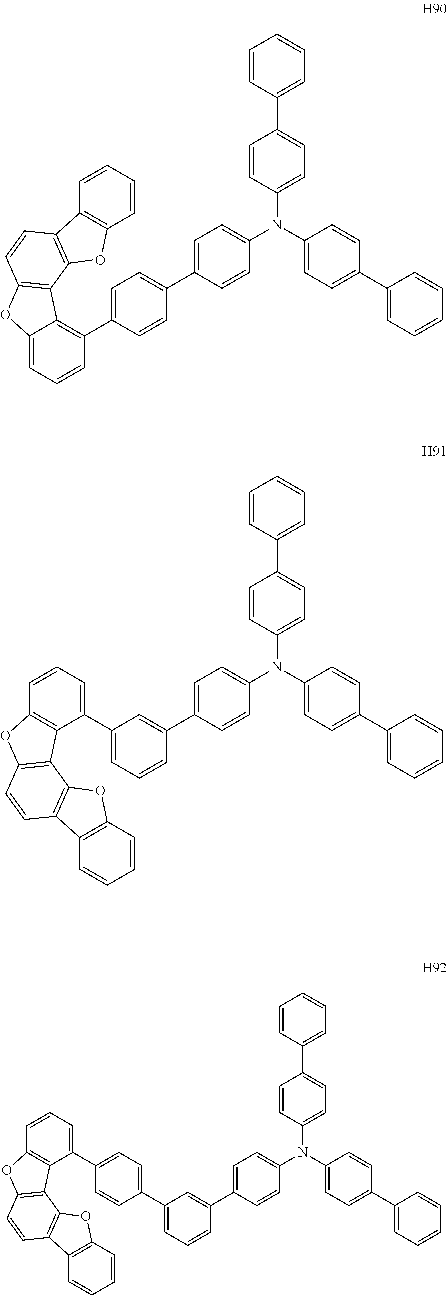 [SMILES: c1ccc(-c2ccc(N(c3ccc(-c4ccccc4)cc3)c3ccc(-c4ccc(-c5cccc6oc7ccc8c9ccccc9oc8c7c56)cc4)cc3)cc2)cc1 ; c1ccc(-c2ccc(N(c3ccc(-c4ccccc4)cc3)c3ccc(-c4cccc(-c5cccc6oc7ccc8c9ccccc9oc8c7c56)c4)cc3)cc2)cc1 ; c1ccc(-c2ccc(N(c3ccc(-c4ccccc4)cc3)c3ccc(-c4cccc(-c5ccc(-c6cccc7oc8ccc9c%10ccccc%10oc9c8c67)cc5)c4)cc3)cc2)cc1]

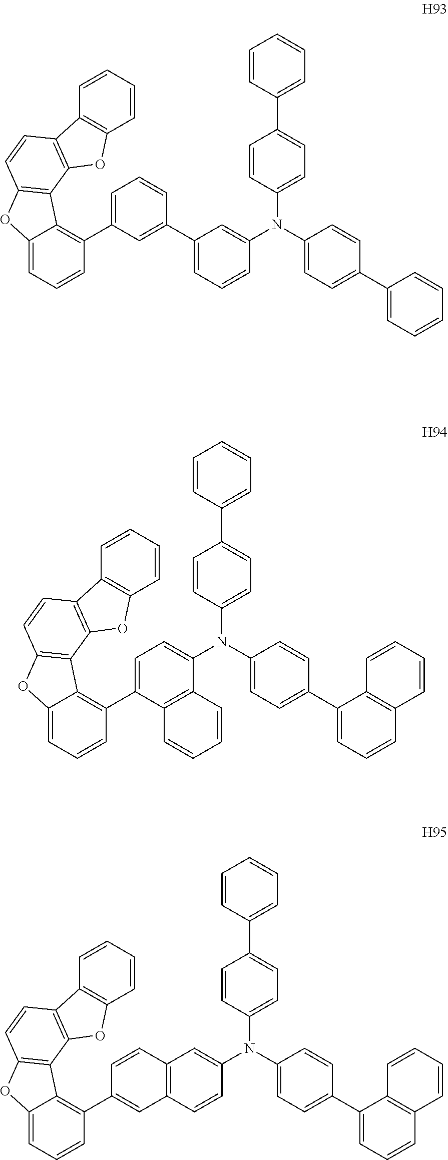 [SMILES: c1ccc(-c2ccc(N(c3ccc(-c4ccccc4)cc3)c3cccc(-c4cccc(-c5cccc6oc7ccc8c9ccccc9oc8c7c56)c4)c3)cc2)cc1 ; c1ccc(-c2ccc(N(c3ccc(-c4cccc5ccccc45)cc3)c3ccc(-c4cccc5oc6ccc7c8ccccc8oc7c6c45)c4ccccc34)cc2)cc1 ; c1ccc(-c2ccc(N(c3ccc(-c4cccc5ccccc45)cc3)c3ccc4cc(-c5cccc6oc7ccc8c9ccccc9oc8c7c56)ccc4c3)cc2)cc1]

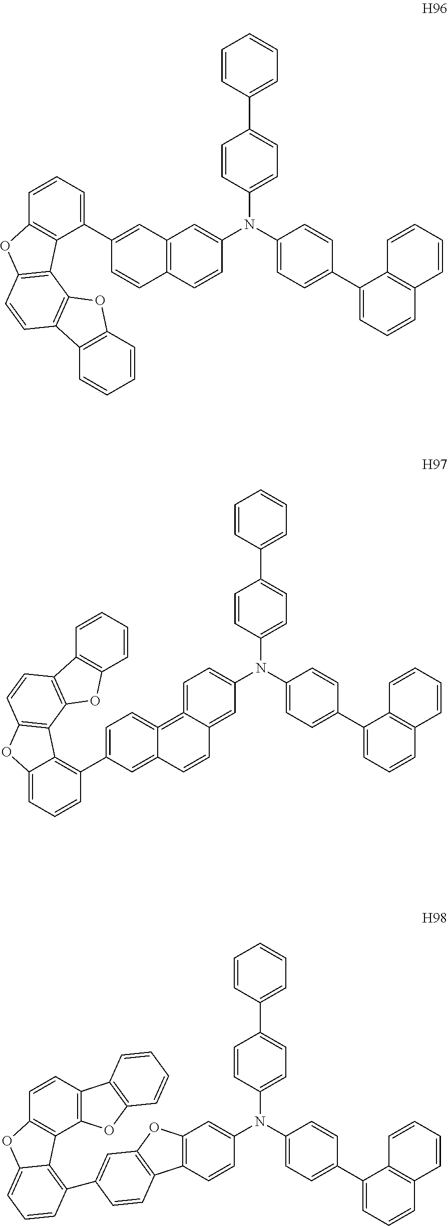

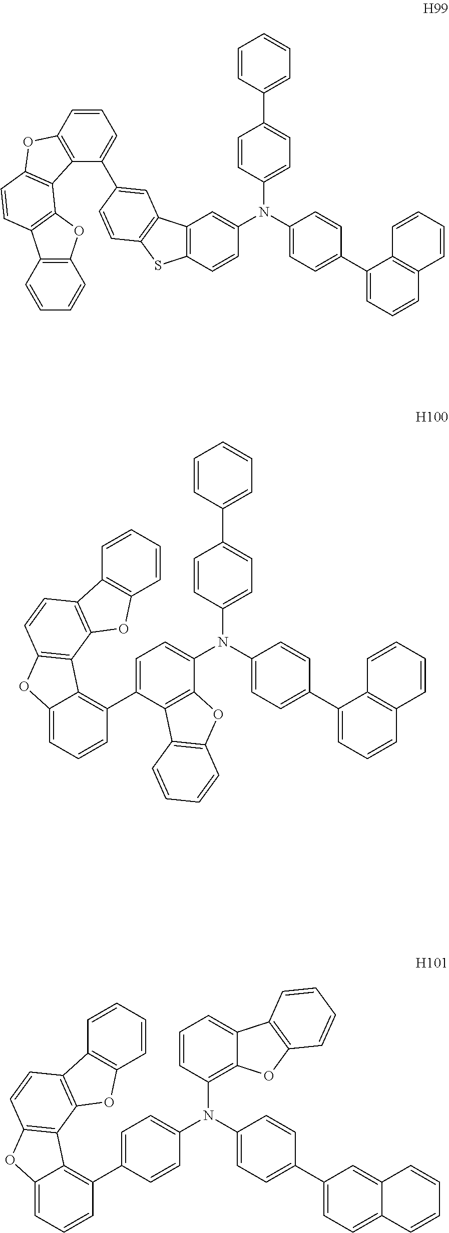 [SMILES: c1ccc(-c2ccc(N(c3ccc(-c4cccc5ccccc45)cc3)c3ccc4sc5ccc(-c6cccc7oc8ccc9c%10ccccc%10oc9c8c67)cc5c4c3)cc2)cc1 ; c1ccc(-c2ccc(N(c3ccc(-c4cccc5ccccc45)cc3)c3ccc(-c4cccc5oc6ccc7c8ccccc8oc7c6c45)c4c3oc3ccccc34)cc2)cc1 ; c1ccc2cc(-c3ccc(N(c4ccc(-c5cccc6oc7ccc8c9ccccc9oc8c7c56)cc4)c4cccc5c4oc4ccccc45)cc3)ccc2c1]

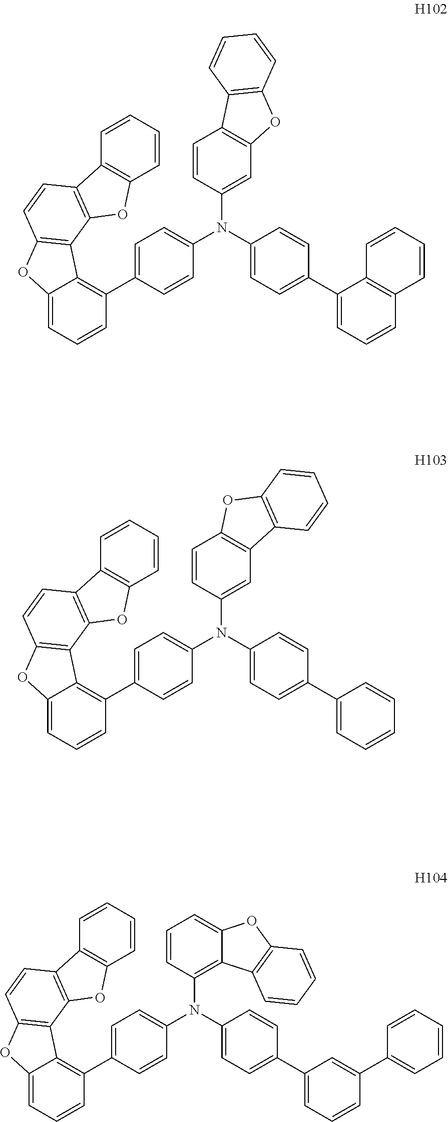 [SMILES: c1ccc2c(-c3ccc(N(c4ccc(-c5cccc6oc7ccc8c9ccccc9oc8c7c56)cc4)c4ccc5c(c4)oc4ccccc45)cc3)cccc2c1 ; c1ccc(-c2ccc(N(c3ccc(-c4cccc5oc6ccc7c8ccccc8oc7c6c45)cc3)c3ccc4oc5ccccc5c4c3)cc2)cc1 ; c1ccc(-c2cccc(-c3ccc(N(c4ccc(-c5cccc6oc7ccc8c9ccccc9oc8c7c56)cc4)c4cccc5oc6ccccc6c45)cc3)c2)cc1]

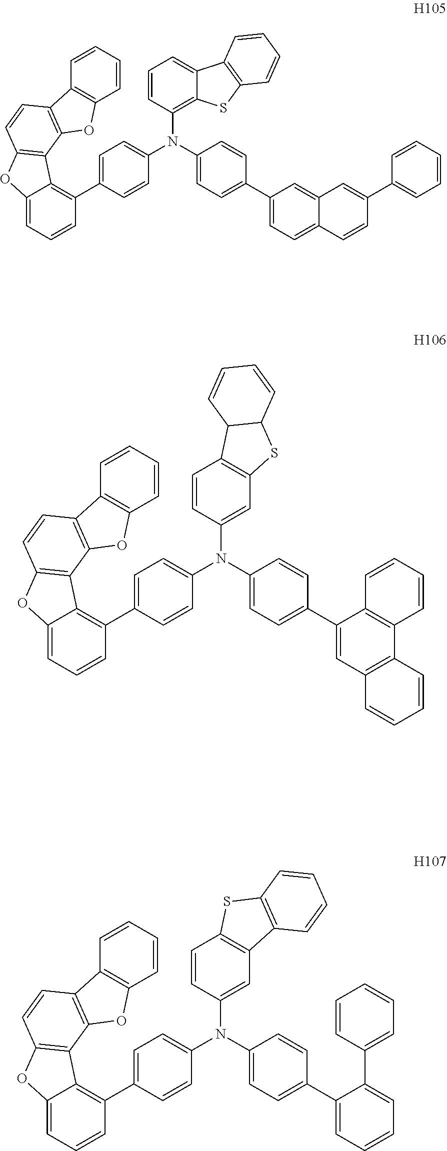 [SMILES: c1ccc(-c2ccc3ccc(-c4ccc(N(c5ccc(-c6cccc7oc8ccc9c%10ccccc%10oc9c8c67)cc5)c5cccc6c5sc5ccccc56)cc4)cc3c2)cc1 ; C1=CC2Sc3cc(N(c4ccc(-c5cc6ccccc6c6ccccc56)cc4)c4ccc(-c5cccc6oc7ccc8c9ccccc9oc8c7c56)cc4)ccc3C2C=C1 ; c1ccc(-c2ccccc2-c2ccc(N(c3ccc(-c4cccc5oc6ccc7c8ccccc8oc7c6c45)cc3)c3ccc4sc5ccccc5c4c3)cc2)cc1]

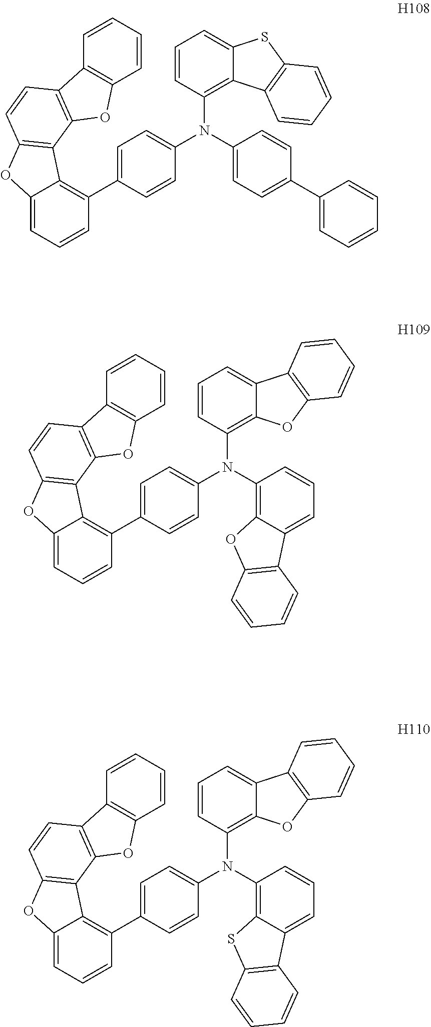 [SMILES: c1ccc(-c2ccc(N(c3ccc(-c4cccc5oc6ccc7c8ccccc8oc7c6c45)cc3)c3cccc4sc5ccccc5c34)cc2)cc1 ; c1ccc2c(c1)oc1c(N(c3ccc(-c4cccc5oc6ccc7c8ccccc8oc7c6c45)cc3)c3cccc4c3oc3ccccc34)cccc12 ; c1ccc2c(c1)oc1c(N(c3ccc(-c4cccc5oc6ccc7c8ccccc8oc7c6c45)cc3)c3cccc4c3sc3ccccc34)cccc12]

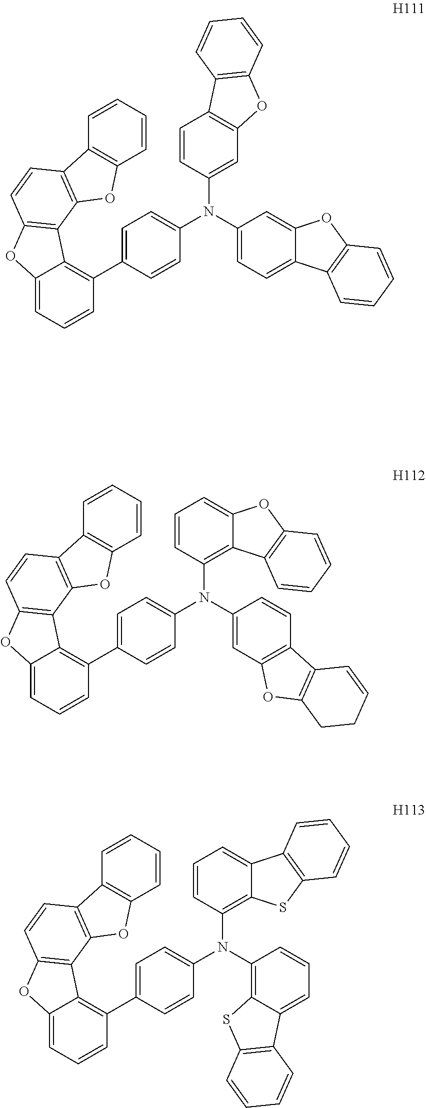 [SMILES: c1ccc2c(c1)oc1cc(N(c3ccc(-c4cccc5oc6ccc7c8ccccc8oc7c6c45)cc3)c3ccc4c(c3)oc3ccccc34)ccc12 ; C1=Cc2c(oc3cc(N(c4ccc(-c5cccc6oc7ccc8c9ccccc9oc8c7c56)cc4)c4cccc5oc6ccccc6c45)ccc23)CC1 ; c1ccc2c(c1)oc1c2ccc2oc3cccc(-c4ccc(N(c5cccc6c5sc5ccccc56)c5cccc6c5sc5ccccc56)cc4)c3c21]

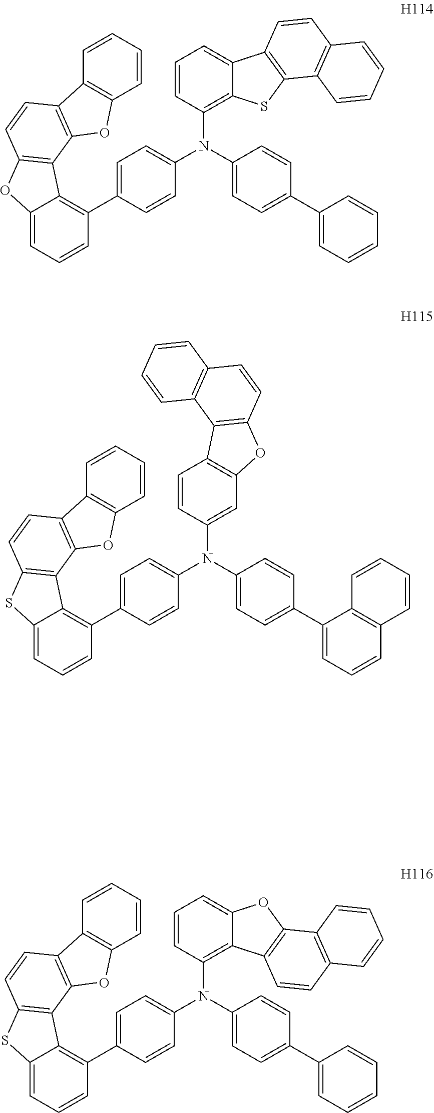 [SMILES: c1ccc(-c2ccc(N(c3ccc(-c4cccc5oc6ccc7c8ccccc8oc7c6c45)cc3)c3cccc4c3sc3c5ccccc5ccc43)cc2)cc1 ; c1ccc2c(-c3ccc(N(c4ccc(-c5cccc6sc7ccc8c9ccccc9oc8c7c56)cc4)c4ccc5c(c4)oc4ccc6ccccc6c45)cc3)cccc2c1 ; c1ccc(-c2ccc(N(c3ccc(-c4cccc5sc6ccc7c8ccccc8oc7c6c45)cc3)c3cccc4oc5c6ccccc6ccc5c34)cc2)cc1]

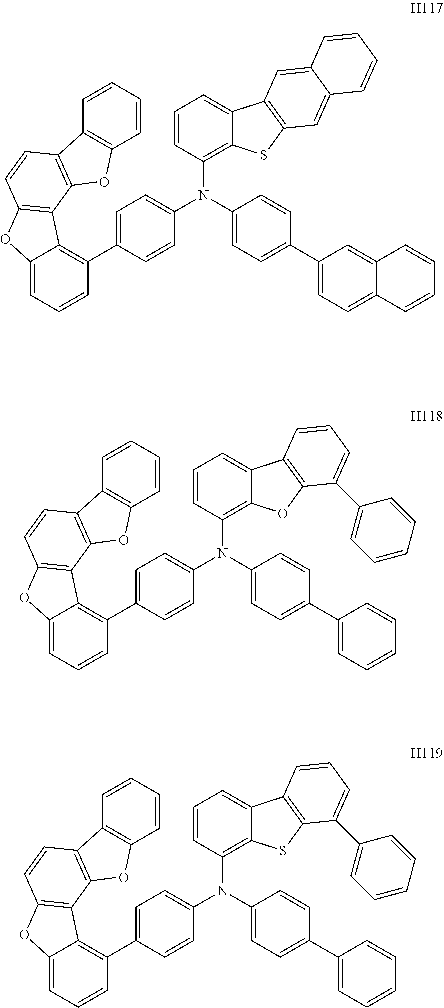 [SMILES: c1ccc2cc(-c3ccc(N(c4ccc(-c5cccc6oc7ccc8c9ccccc9oc8c7c56)cc4)c4cccc5c4sc4cc6ccccc6cc45)cc3)ccc2c1 ; c1ccc(-c2ccc(N(c3ccc(-c4cccc5oc6ccc7c8ccccc8oc7c6c45)cc3)c3cccc4c3oc3c(-c5ccccc5)cccc34)cc2)cc1 ; c1ccc(-c2ccc(N(c3ccc(-c4cccc5oc6ccc7c8ccccc8oc7c6c45)cc3)c3cccc4c3sc3c(-c5ccccc5)cccc34)cc2)cc1]

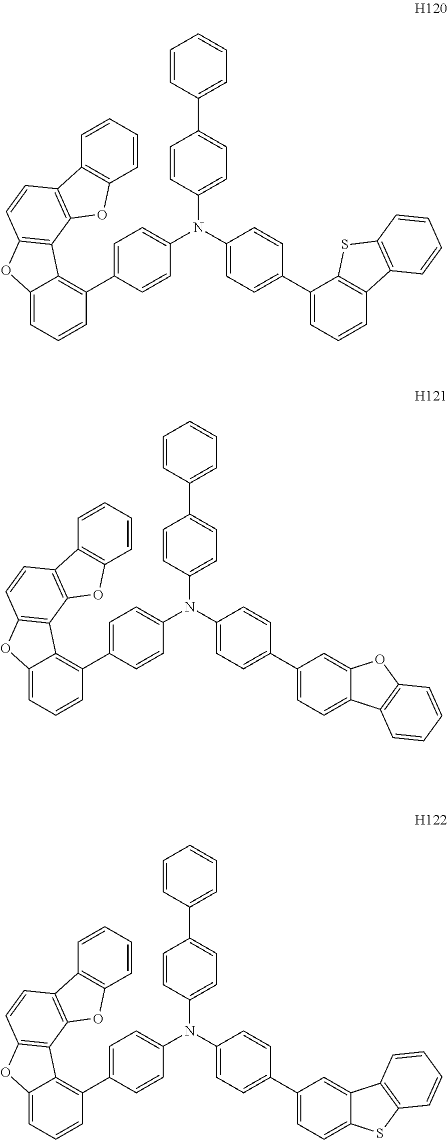 [SMILES: c1ccc(-c2ccc(N(c3ccc(-c4cccc5c4sc4ccccc45)cc3)c3ccc(-c4cccc5oc6ccc7c8ccccc8oc7c6c45)cc3)cc2)cc1 ; c1ccc(-c2ccc(N(c3ccc(-c4ccc5c(c4)oc4ccccc45)cc3)c3ccc(-c4cccc5oc6ccc7c8ccccc8oc7c6c45)cc3)cc2)cc1 ; c1ccc(-c2ccc(N(c3ccc(-c4ccc5sc6ccccc6c5c4)cc3)c3ccc(-c4cccc5oc6ccc7c8ccccc8oc7c6c45)cc3)cc2)cc1]

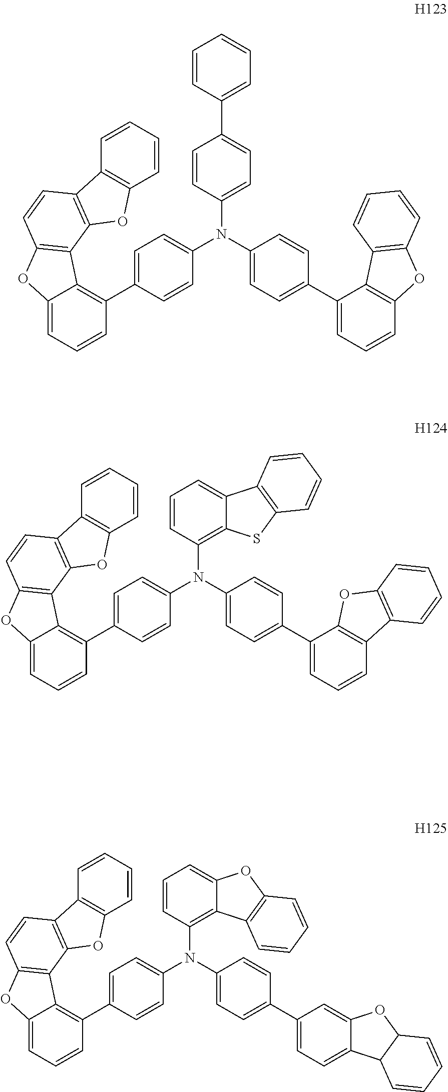 [SMILES: c1ccc(-c2ccc(N(c3ccc(-c4cccc5oc6ccccc6c45)cc3)c3ccc(-c4cccc5oc6ccc7c8ccccc8oc7c6c45)cc3)cc2)cc1 ; c1ccc2c(c1)oc1c(-c3ccc(N(c4ccc(-c5cccc6oc7ccc8c9ccccc9oc8c7c56)cc4)c4cccc5c4sc4ccccc45)cc3)cccc12 ; C1=CC2Oc3cc(-c4ccc(N(c5ccc(-c6cccc7oc8ccc9c%10ccccc%10oc9c8c67)cc5)c5cccc6oc7ccccc7c56)cc4)ccc3C2C=C1]

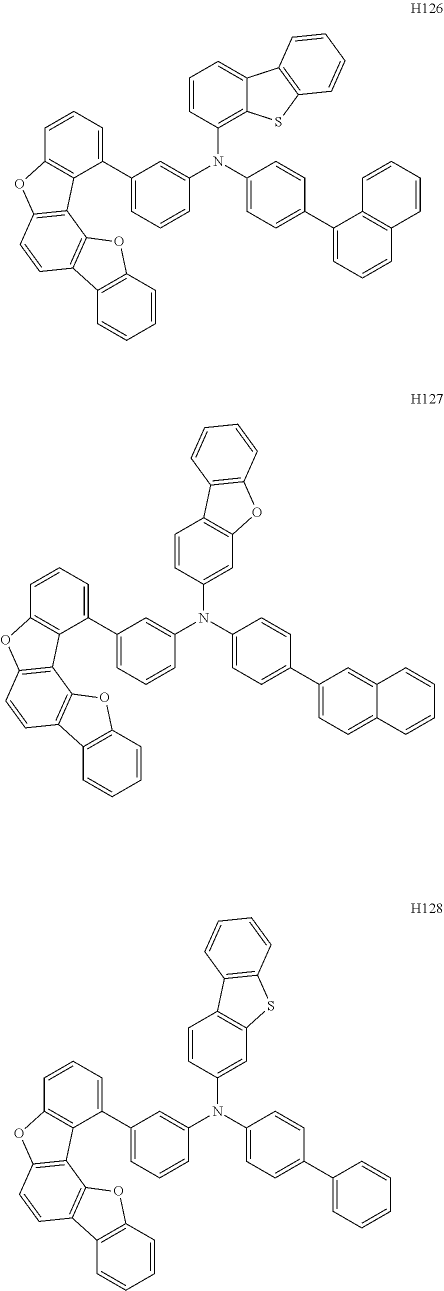 [SMILES: c1cc(-c2cccc3oc4ccc5c6ccccc6oc5c4c23)cc(N(c2ccc(-c3cccc4ccccc34)cc2)c2cccc3c2sc2ccccc23)c1 ; c1cc(-c2cccc3oc4ccc5c6ccccc6oc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5oc6ccc7c8ccccc8oc7c6c45)c3)c3ccc4c(c3)sc3ccccc34)cc2)cc1]

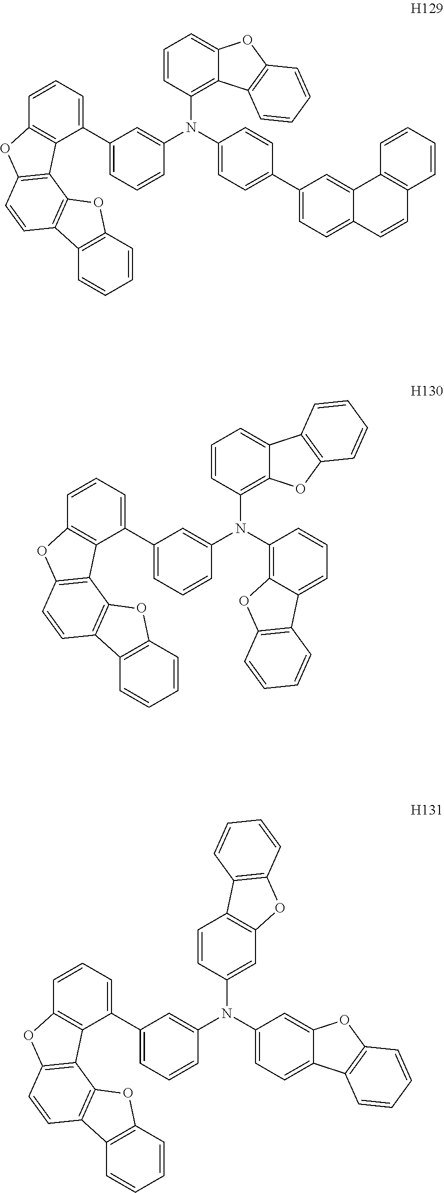 [SMILES: c1cc(-c2cccc3oc4ccc5c6ccccc6oc5c4c23)cc(N(c2ccc(-c3ccc4ccc5ccccc5c4c3)cc2)c2cccc3oc4ccccc4c23)c1 ; c1cc(-c2cccc3oc4ccc5c6ccccc6oc5c4c23)cc(N(c2cccc3c2oc2ccccc23)c2cccc3c2oc2ccccc23)c1 ; c1cc(-c2cccc3oc4ccc5c6ccccc6oc5c4c23)cc(N(c2ccc3c(c2)oc2ccccc23)c2ccc3c(c2)oc2ccccc23)c1]

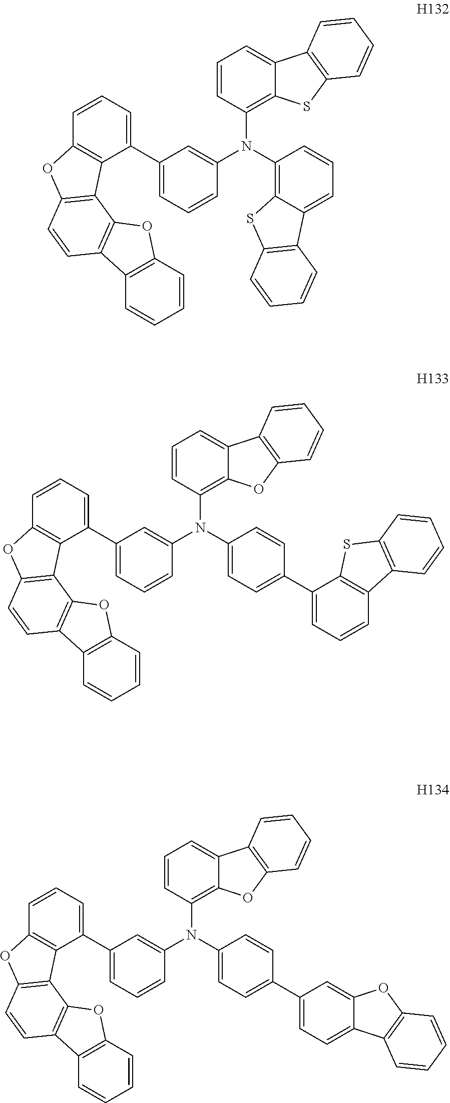 [SMILES: c1cc(-c2cccc3oc4ccc5c6ccccc6oc5c4c23)cc(N(c2cccc3c2sc2ccccc23)c2cccc3c2sc2ccccc23)c1 ; c1cc(-c2cccc3oc4ccc5c6ccccc6oc5c4c23)cc(N(c2ccc(-c3cccc4c3sc3ccccc34)cc2)c2cccc3c2oc2ccccc23)c1 ; c1cc(-c2cccc3oc4ccc5c6ccccc6oc5c4c23)cc(N(c2ccc(-c3ccc4c(c3)oc3ccccc34)cc2)c2cccc3c2oc2ccccc23)c1]

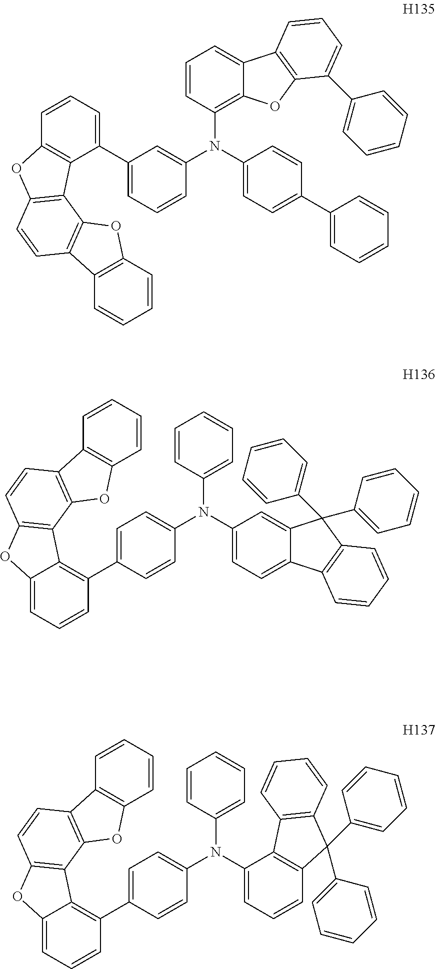 [SMILES: c1ccc(-c2ccc(N(c3cccc(-c4cccc5oc6ccc7c8ccccc8oc7c6c45)c3)c3cccc4c3oc3c(-c5ccccc5)cccc34)cc2)cc1 ; c1ccc(N(c2ccc(-c3cccc4oc5ccc6c7ccccc7oc6c5c34)cc2)c2ccc3c(c2)C(c2ccccc2)(c2ccccc2)c2ccccc2-3)cc1 ; c1ccc(N(c2ccc(-c3cccc4oc5ccc6c7ccccc7oc6c5c34)cc2)c2cccc3c2-c2ccccc2C3(c2ccccc2)c2ccccc2)cc1]

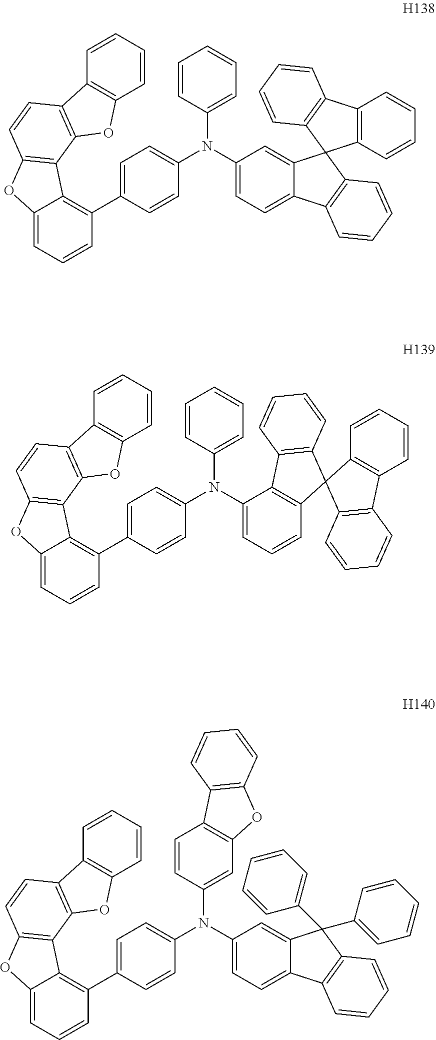 [SMILES: c1ccc(N(c2ccc(-c3cccc4oc5ccc6c7ccccc7oc6c5c34)cc2)c2ccc3c(c2)C2(c4ccccc4-c4ccccc42)c2ccccc2-3)cc1 ; c1ccc(N(c2ccc(-c3cccc4oc5ccc6c7ccccc7oc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6oc7ccc8c9ccccc9oc8c7c56)cc4)c4ccc5c(c4)oc4ccccc45)cc32)cc1]

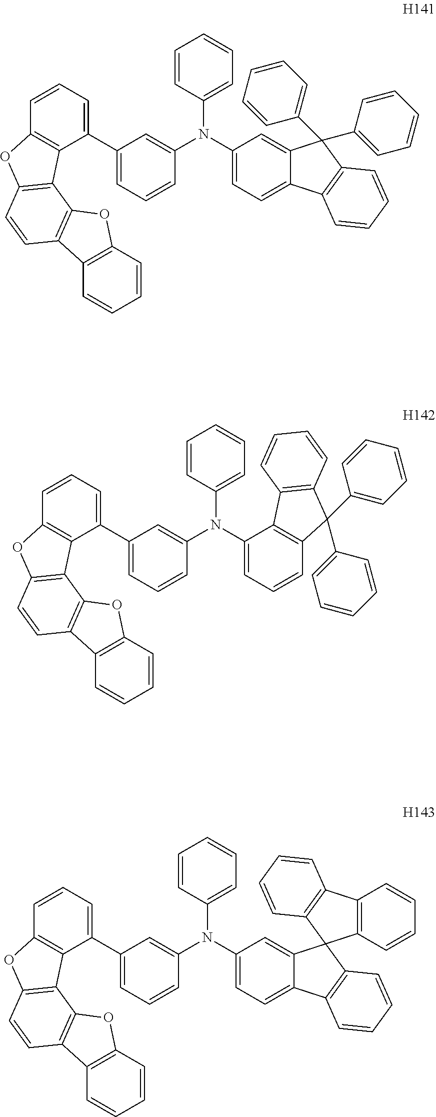 [SMILES: c1ccc(N(c2cccc(-c3cccc4oc5ccc6c7ccccc7oc6c5c34)c2)c2ccc3c(c2)C(c2ccccc2)(c2ccccc2)c2ccccc2-3)cc1 ; c1ccc(N(c2cccc(-c3cccc4oc5ccc6c7ccccc7oc6c5c34)c2)c2cccc3c2-c2ccccc2C3(c2ccccc2)c2ccccc2)cc1 ; c1ccc(N(c2cccc(-c3cccc4oc5ccc6c7ccccc7oc6c5c34)c2)c2ccc3c(c2)C2(c4ccccc4-c4ccccc42)c2ccccc2-3)cc1]

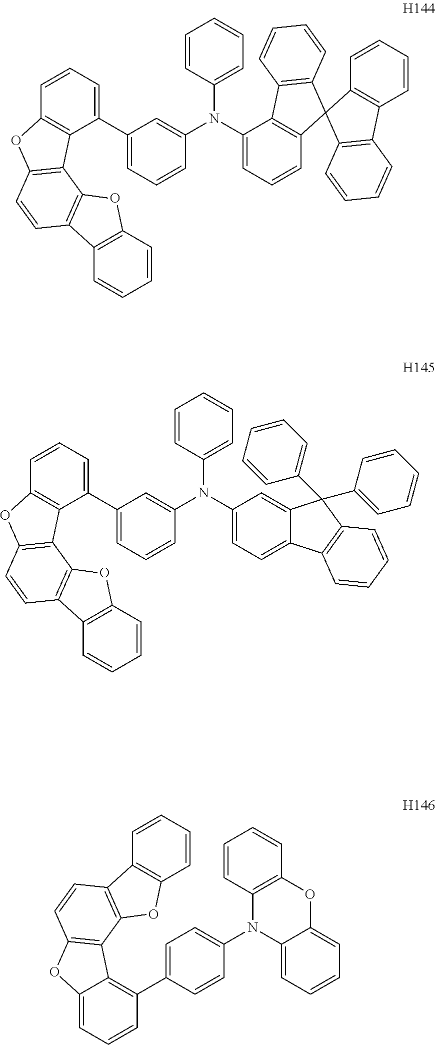 [SMILES: c1ccc(N(c2cccc(-c3cccc4oc5ccc6c7ccccc7oc6c5c34)c2)c2cccc3c2-c2ccccc2C32c3ccccc3-c3ccccc32)cc1 ; c1ccc(N(c2cccc(-c3cccc4oc5ccc6c7ccccc7oc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3oc4ccc5c6ccccc6oc5c4c23)cc1]

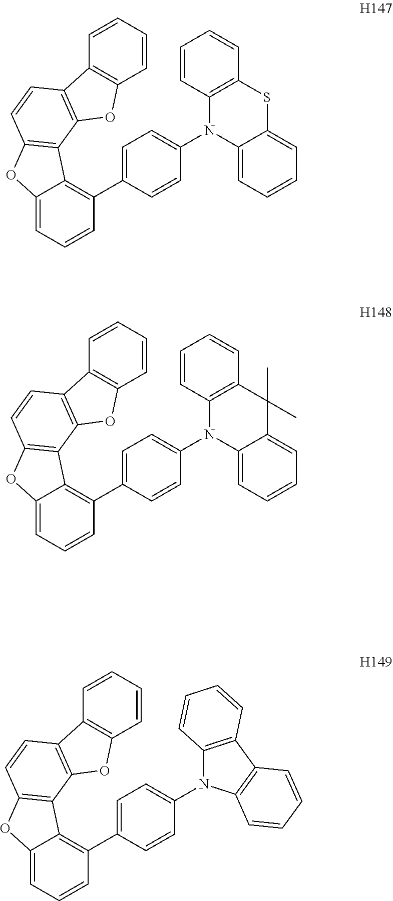 [SMILES: c1ccc2c(c1)Sc1ccccc1N2c1ccc(-c2cccc3oc4ccc5c6ccccc6oc5c4c23)cc1 ; CC1(C)c2ccccc2N(c2ccc(-c3cccc4oc5ccc6c7ccccc7oc6c5c34)cc2)c2ccccc21 ; c1ccc2c(c1)oc1c2ccc2oc3cccc(-c4ccc(-n5c6ccccc6c6ccccc65)cc4)c3c21]

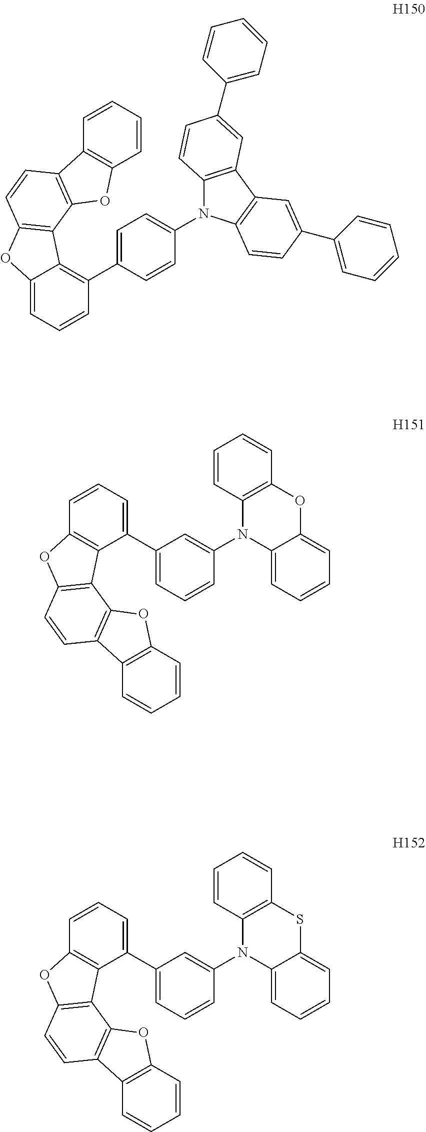 [SMILES: c1ccc(-c2ccc3c(c2)c2cc(-c4ccccc4)ccc2n3-c2ccc(-c3cccc4oc5ccc6c7ccccc7oc6c5c34)cc2)cc1 ; c1cc(-c2cccc3oc4ccc5c6ccccc6oc5c4c23)cc(N2c3ccccc3Oc3ccccc32)c1 ; c1cc(-c2cccc3oc4ccc5c6ccccc6oc5c4c23)cc(N2c3ccccc3Sc3ccccc32)c1]

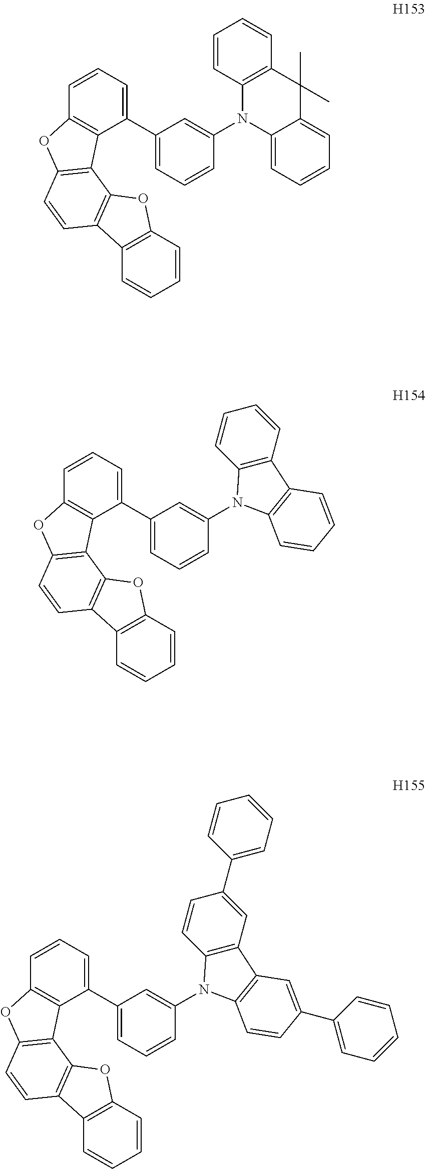 [SMILES: CC1(C)c2ccccc2N(c2cccc(-c3cccc4oc5ccc6c7ccccc7oc6c5c34)c2)c2ccccc21 ; c1cc(-c2cccc3oc4ccc5c6ccccc6oc5c4c23)cc(-n2c3ccccc3c3ccccc32)c1 ; c1ccc(-c2ccc3c(c2)c2cc(-c4ccccc4)ccc2n3-c2cccc(-c3cccc4oc5ccc6c7ccccc7oc6c5c34)c2)cc1]

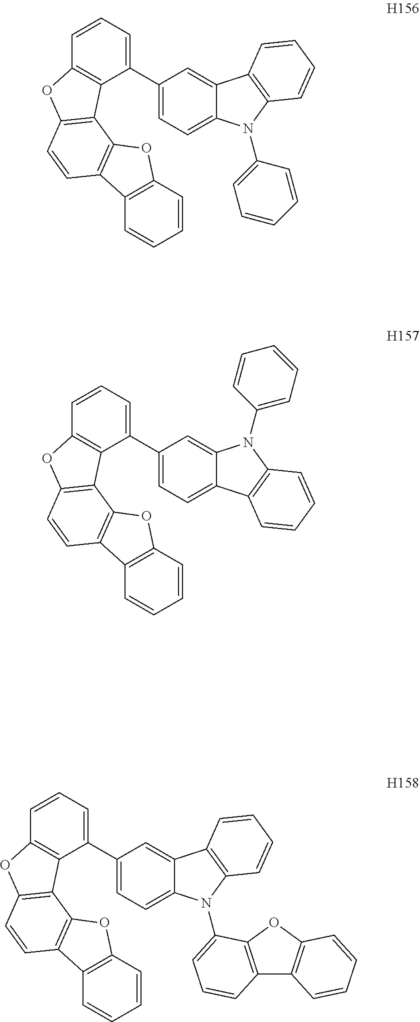 [SMILES: c1ccc(-n2c3ccccc3c3cc(-c4cccc5oc6ccc7c8ccccc8oc7c6c45)ccc32)cc1 ; c1ccc(-n2c3ccccc3c3ccc(-c4cccc5oc6ccc7c8ccccc8oc7c6c45)cc32)cc1 ; c1ccc2c(c1)oc1c(-n3c4ccccc4c4cc(-c5cccc6oc7ccc8c9ccccc9oc8c7c56)ccc43)cccc12]

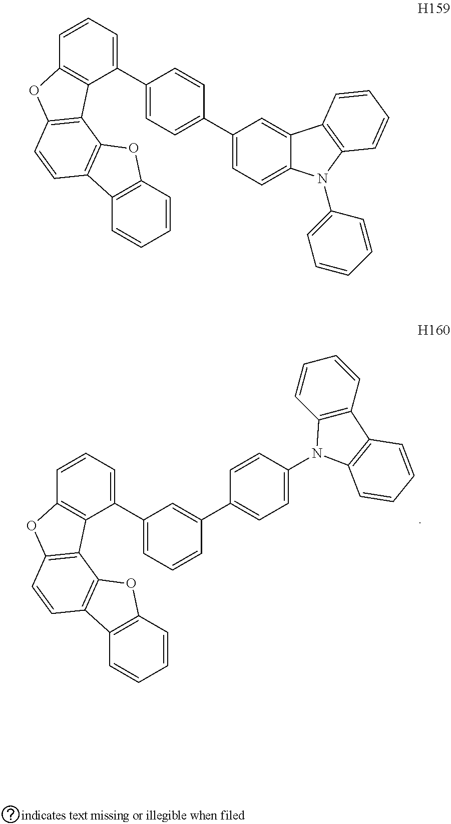 [SMILES: c1ccc(-n2c3ccccc3c3cc(-c4ccc(-c5cccc6oc7ccc8c9ccccc9oc8c7c56)cc4)ccc32)cc1 ; c1cc(-c2ccc(-n3c4ccccc4c4ccccc43)cc2)cc(-c2cccc3oc4ccc5c6ccccc6oc5c4c23)c1]

The polycyclic compound represented by Formula 1 or Formula A according to an embodiment may have a molecular structure in which the benzobisdibenzoheterol moiety and the amine derivative moiety are bonded to thus form a functional layer having excellent film properties due to the three-dimensional molecular structure, thereby contributing to the improvement of a service life and efficiency of the light emitting device. The polycyclic compound represented by Formula 1 or Formula A according to an embodiment may exhibit characteristics having improved stability and hole transport characteristics of materials since the skeletal structure of the benzobisdibenzoheterol moiety is specified, and the bonding position of the nitrogen atom of the amine derivative moiety and the benzobisdibenzoheterol moiety is specified.

For example, when the polycyclic compound of an embodiment is used in the hole transport region, the hole transport characteristic may be increased to improve recombination probability of holes and electrons in the emission layer, thereby improving luminous efficiency. The polycyclic compound of an embodiment, which has a structure in which the benzobisdibenzoheterol moiety and the amine derivative moiety are bonded at a specific position as described above to thus have excellent stability, is included as a material for the light emitting device, and thereby a service life of the light emitting device of an embodiment may also be improved.

The light emitting device ED of an embodiment may further include materials for the hole transport region, which will be described below, with the polycyclic compound of an embodiment as described above.

The hole transport region HTR may include a compound represented by Formula H-1 below:

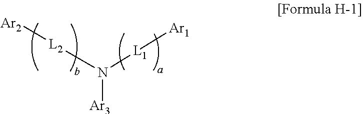

In Formula H-1 above, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-1, a and b may each independently be an integer from 0 to 10. When a or b is an integer of 2 or greater, multiple L₁(s) and L₂(s) may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In Formula H-1, Ar₁₁ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 above may be a monoamine compound. In other embodiments, the compound represented by Formula H-1 above may be a diamine compound in which at least one among Ar₁ to Ar₁₁ includes an amine group as a substituent. The compound represented by Formula H-1 above may be a carbazole-based compound including a substituted or unsubstituted carbazole group in at least one of Ar₁ and Ar₂, or a fluorene-based compound including a substituted or unsubstituted fluorene group in at least one of Ar₁ and Ar₂.

The compound represented by Formula H-1 may be represented by any one among the compounds of Compound Group H below. However, the compounds listed in Compound Group H below are examples, and the compounds represented by Formula H-1 are not limited to those represented by Compound Group H below:

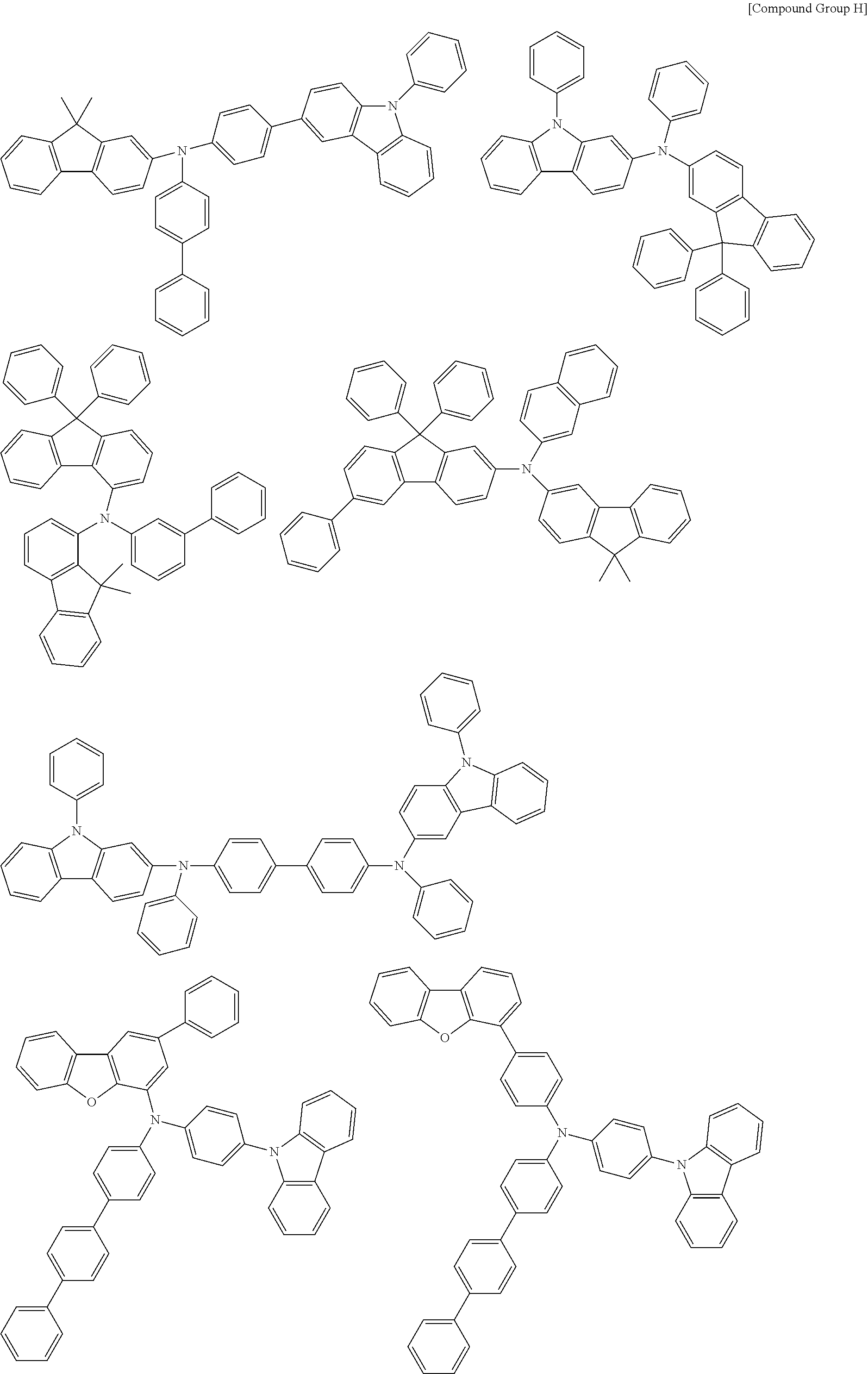

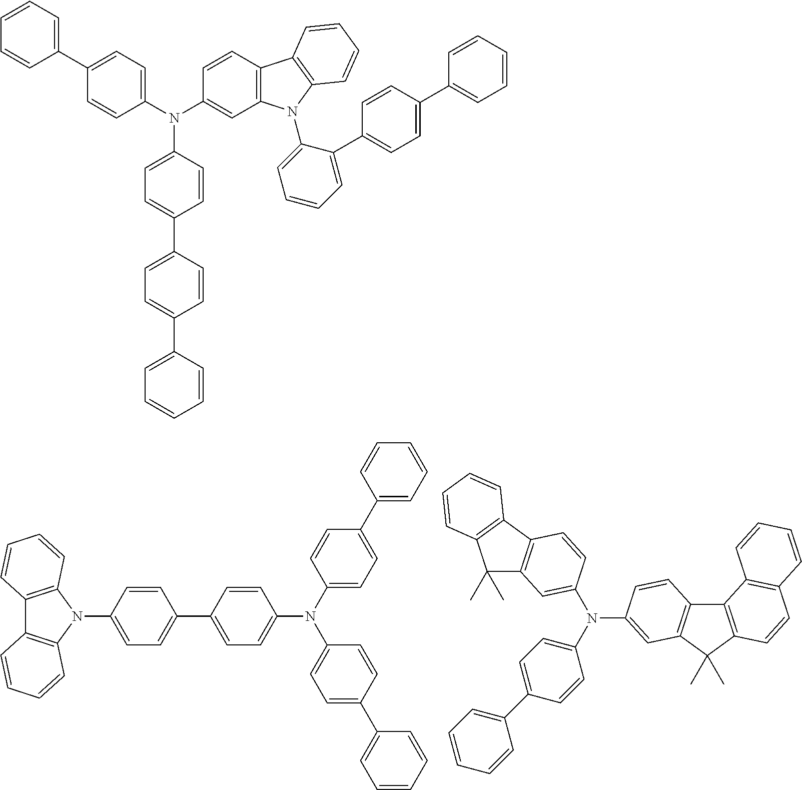

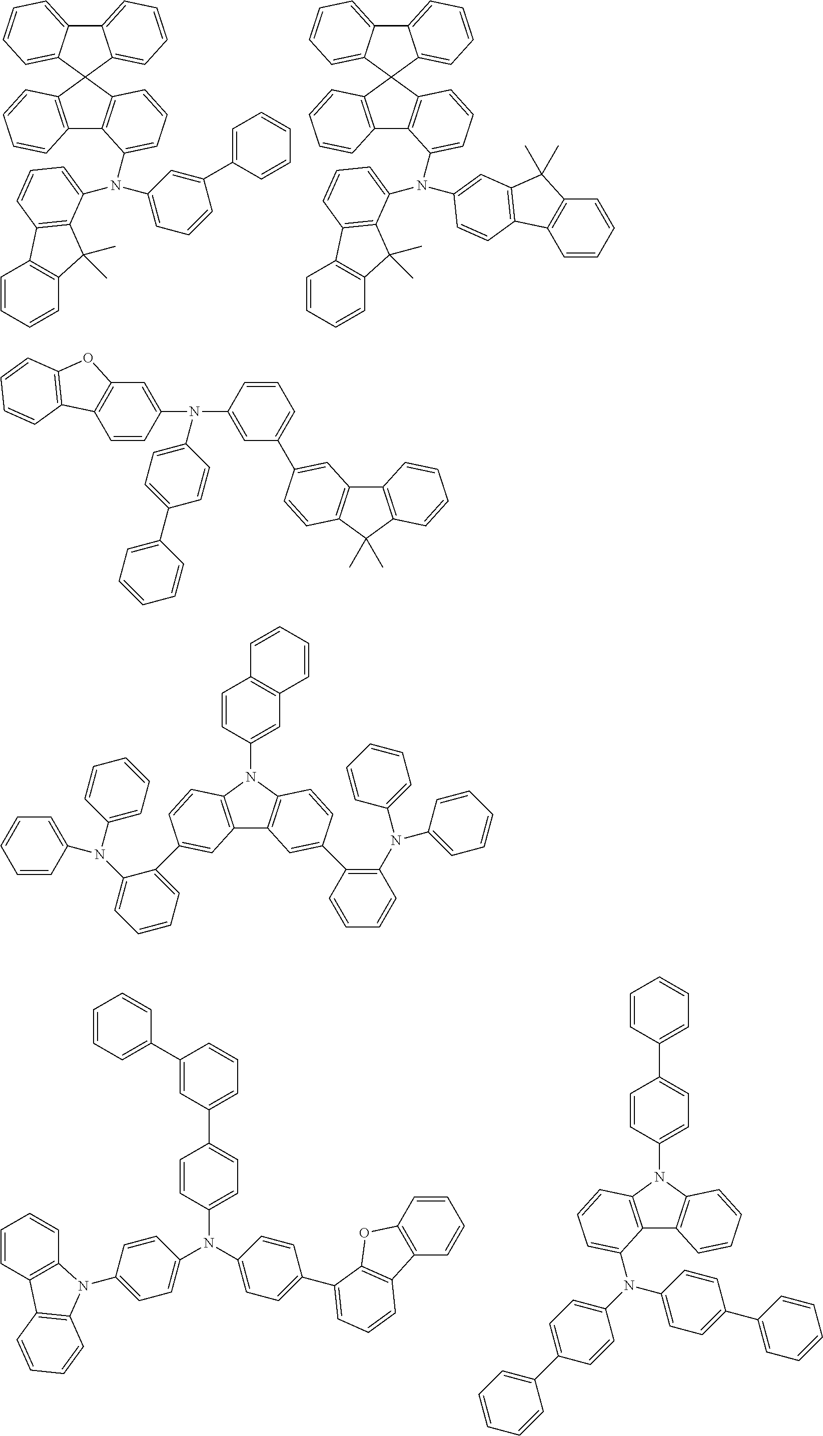

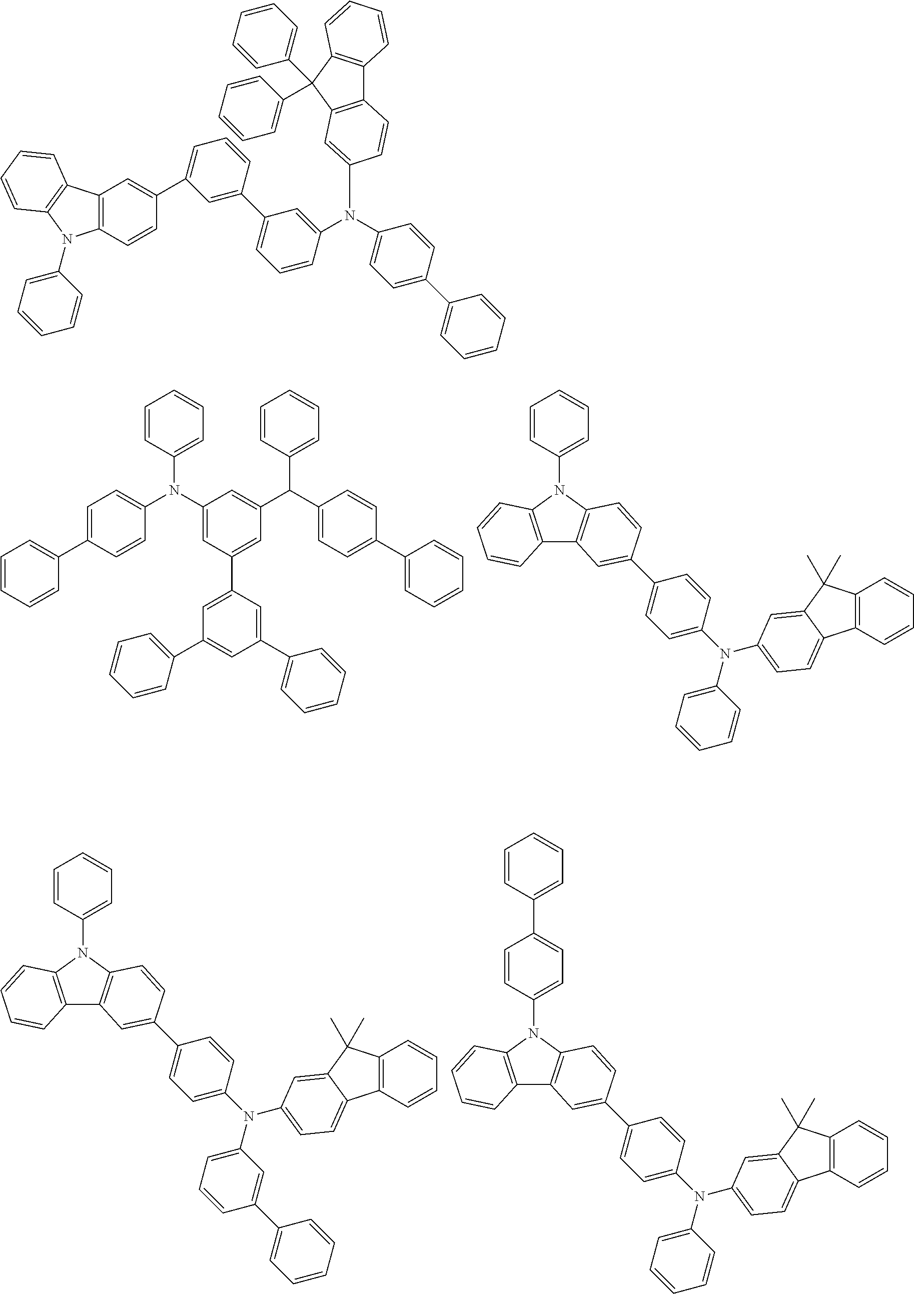

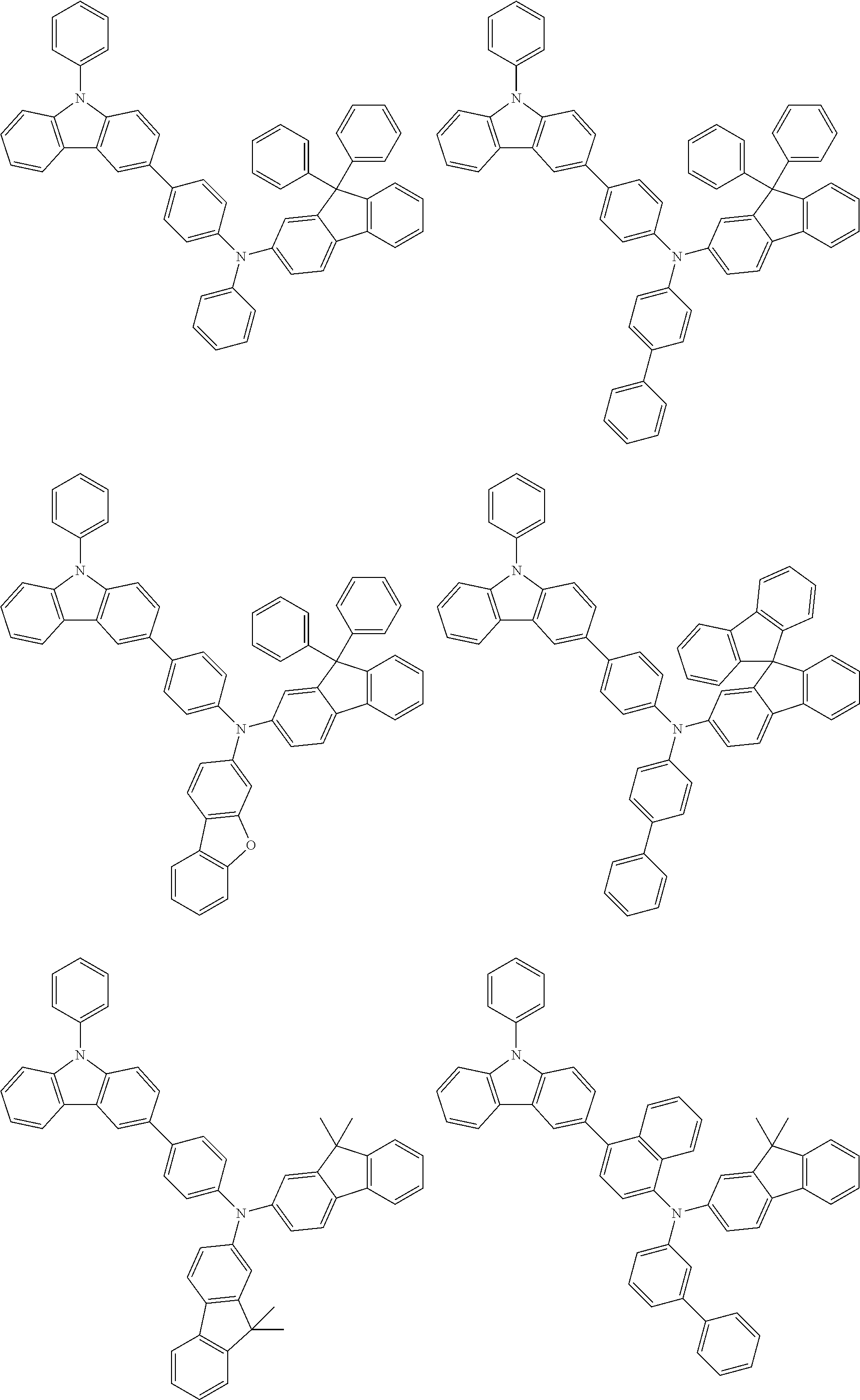

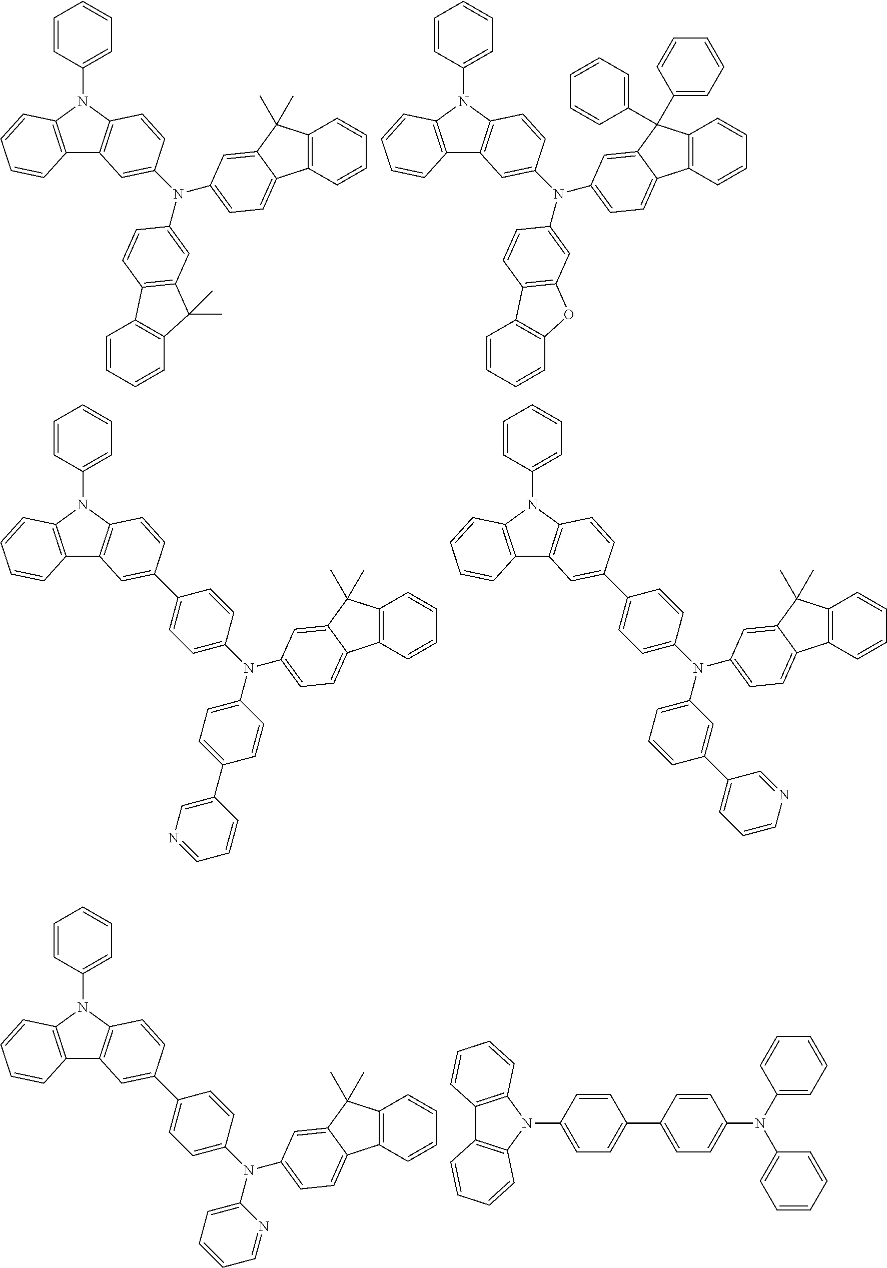

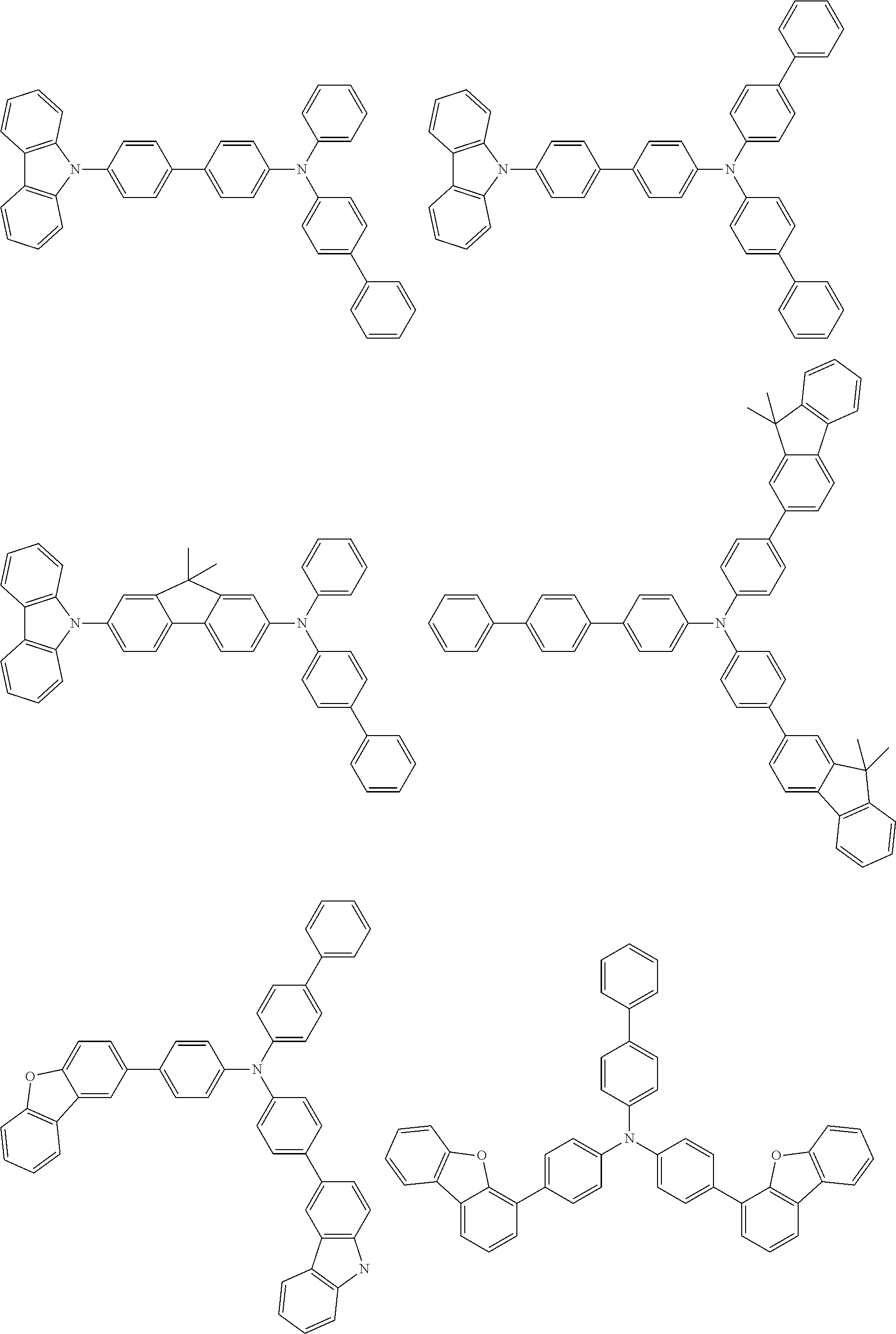

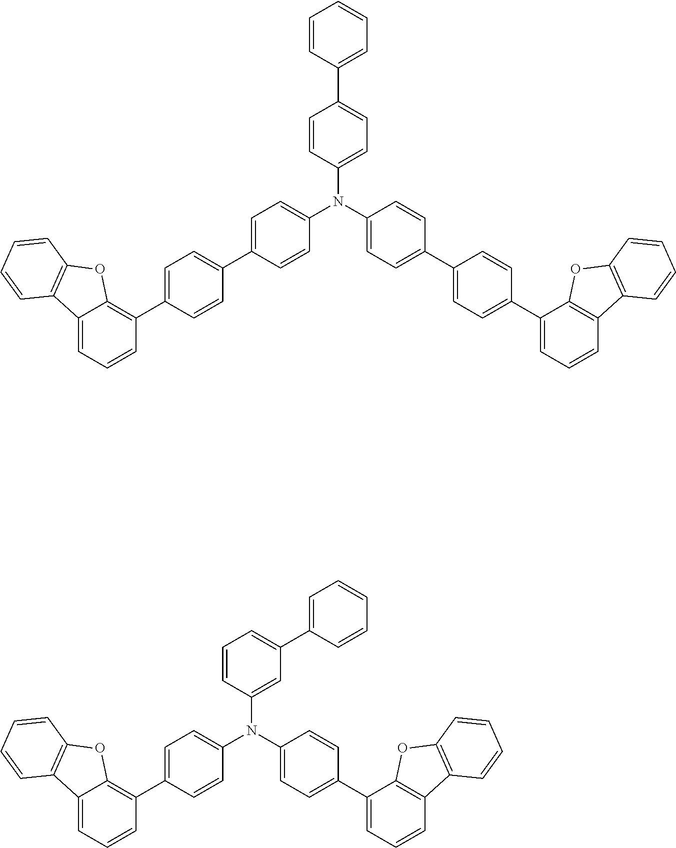 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1]

The hole transport region HTR may include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-Tris(N,N-diphenyl amino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include carbazole derivatives such as N-phenyl carbazole and polyvinyl carbazole, fluorene derivatives, N,N′-bis(3-methylphenyl)-N,N′-diphenyl-[1,1-biphenyl]-4,4′-diamine (TPD), triphenylamine derivatives such as 4,4′,4″-tris(N-carbazolyl)triphenylamine (TCTA), N,N′-di(naphthalene-1-yl)-N,N′-diplienyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), 1,3-bis(1,8-dimethyl-9H-carbazol-9-yl)benzene (mDCP), etc.

The hole transport region HTR may include the above-described compound of the hole transport region in at least one of a hole injection layer HIL, a hole transport layer HTL, and an electron blocking layer EBL.

A thickness of the hole transport region HTR may be in a range of about 100 Å to about 10,000 Å. For example, the thickness of the hole transport region HTR may be in a range of about 100 Å to about 5,000 Å. When the hole transport region HTR includes a hole injection layer HIL, the hole injection layer HIL may have, for example, a thickness in a range of about 30 Å to about 1,000 Å. When the hole transport region HTR includes a hole transport layer HTL, the hole transport layer HTL may have a thickness in a range of about 30 Å to about 1,000 Å. For example, when the hole transport region HTR includes an electron blocking layer EBL, the electron blocking layer EBL may have a thickness in a range of about 10 Å to about 1,000 Å. If the thicknesses of the hole transport region HTR, the hole injection layer HIL, the hole transport layer HTL and the electron blocking layer EBL satisfy the above-described ranges, satisfactory hole transport characteristics may be achieved without a substantial increase in a driving voltage.

The hole transport region HTR may further include a charge generating material in addition to the above-described materials to increase conductivity. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, and a cyano group-containing compound, but embodiments are not limited thereto. For example, the p-dopant may include metal halides such as CuI and RbI, quinone derivatives such as tetracyanoquinodimethane (TCNQ) and 2,3,5,6-tetrafluoro-7,7,8,8-tetracyanoquinodimethane (F4-TCNQ), metal oxides such as tungsten oxide and molybdenum oxide, dipyrazino[2,3-f: 2′,3′-h] quinoxaline-2,3,6,7,10,11-hexacarbonitrile (HATCN), 4-[[2,3-bis[cyano-(4-cyano-2,3,5,6-tetrafluorophenyl)methylidene]cyclopropylidene]-cyanometh yl]-2,3,5,6-tetrafluorobenzonitrile, etc., but embodiments are not limited thereto.

As described above, the hole transport region HTR may further include at least one of the buffer layer (not shown) and the electron blocking layer EBL in addition to the hole injection layer HIL and the hole transport layer HTL. The buffer layer (not shown) may compensate a resonance distance according to the wavelength of light emitted from the emission layer EML and may thus increase light emission efficiency. Materials which may be included in the hole transport region HTR may be used as materials to be included in the buffer layer (not shown). The electron blocking layer EBL is a layer that serves to prevent electrons from being injected from the electron transport region ETR to the hole transport region HTR.

The emission layer EML is provided on the hole transport region HTR. The emission layer EML may have a thickness in a range of, for example, about 100 Å to about 1,000 Å. For example, the thickness of emission layer EML may be in a range of about 100 Å to about 300 Å. The emission layer EML may have a single layer formed of a single material, a single layer formed of different materials, or a multilayer structure having multiple layers formed of different materials.

In the light emitting device ED of an embodiment, the emission layer EML may include anthracene derivatives, pyrene derivatives, fluoranthene derivatives, chrysene derivatives, dihydrobenzanthrene derivatives, or triphenylene derivatives. For example, the emission layer EML may include anthracene derivatives or pyrene derivatives.

In each light emitting device ED of embodiments illustrated in FIGS. 3 to 6, the emission layer EML may include a host and a dopant, and the emission layer EML may include a compound represented by Formula E-1 below. The compound represented by Formula E-1 below may be used as a fluorescence host material.

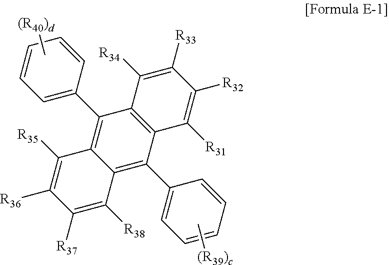

In Formula E-1, R₃₁ to R₄₀ may each independently be a hydrogen atom, a deuterium atom, a halogen atom, a substituted or unsubstituted silyl group, a substituted or unsubstituted alkyl group having 1 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula E-1, R₃₁ to R₄₀ may be bonded to an adjacent group to form a saturated hydrocarbon ring or an unsaturated hydrocarbon ring.

In Formula E-1, c and d may each independently be an integer from 0 to 5.

Formula E-1 may be represented by any one among Compound E1 to Compound E19 below:

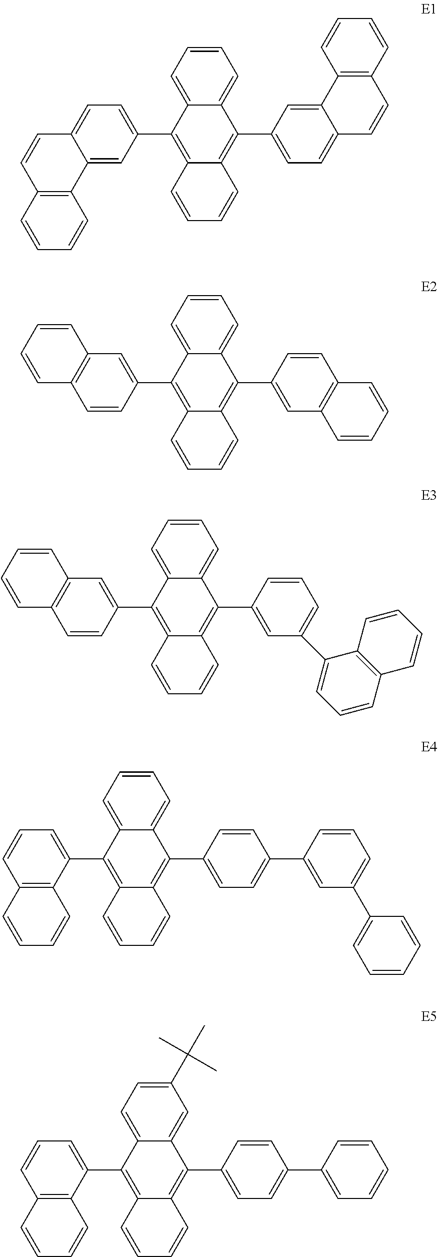

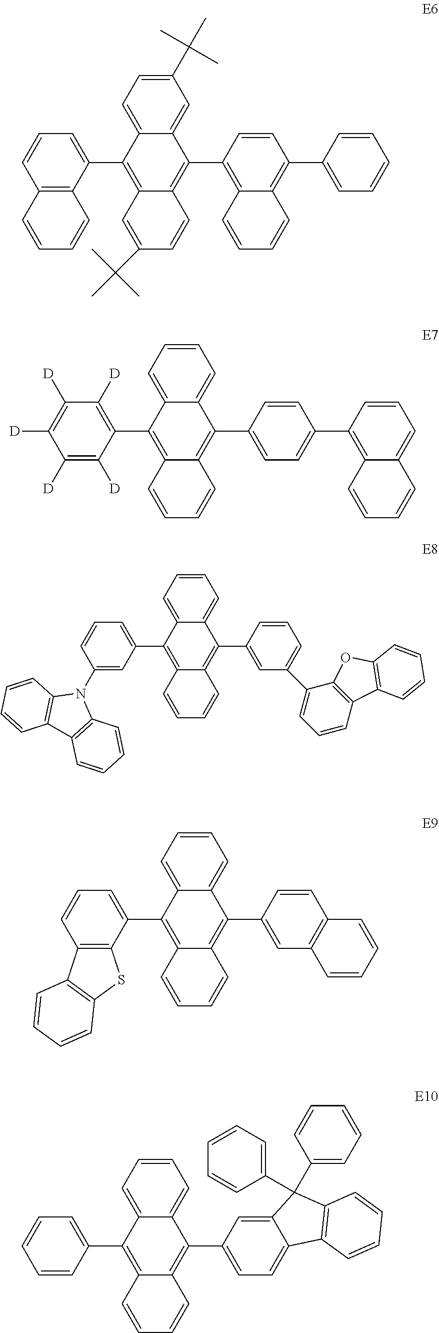

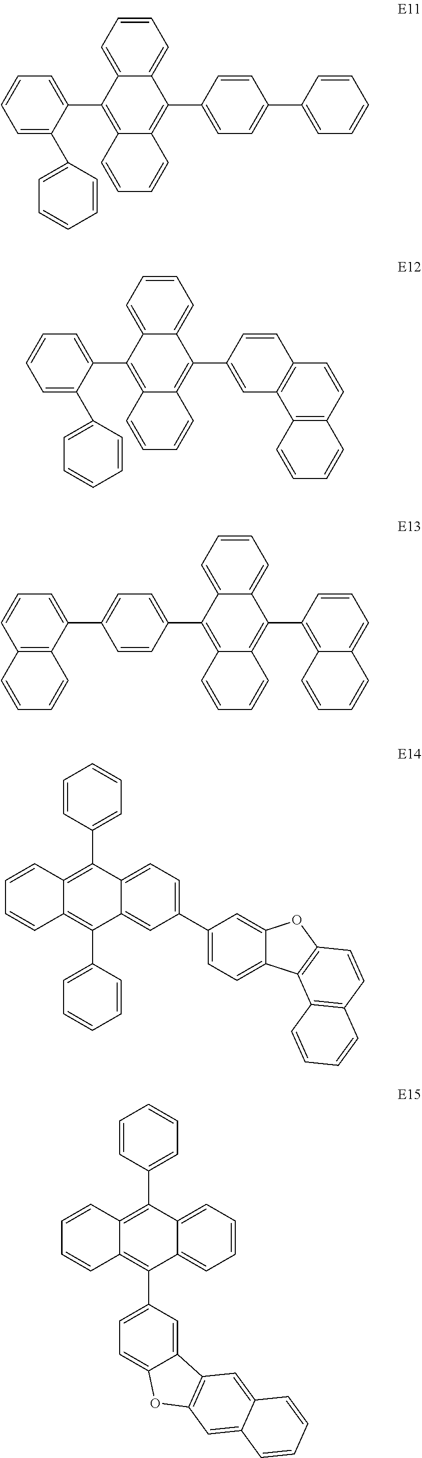

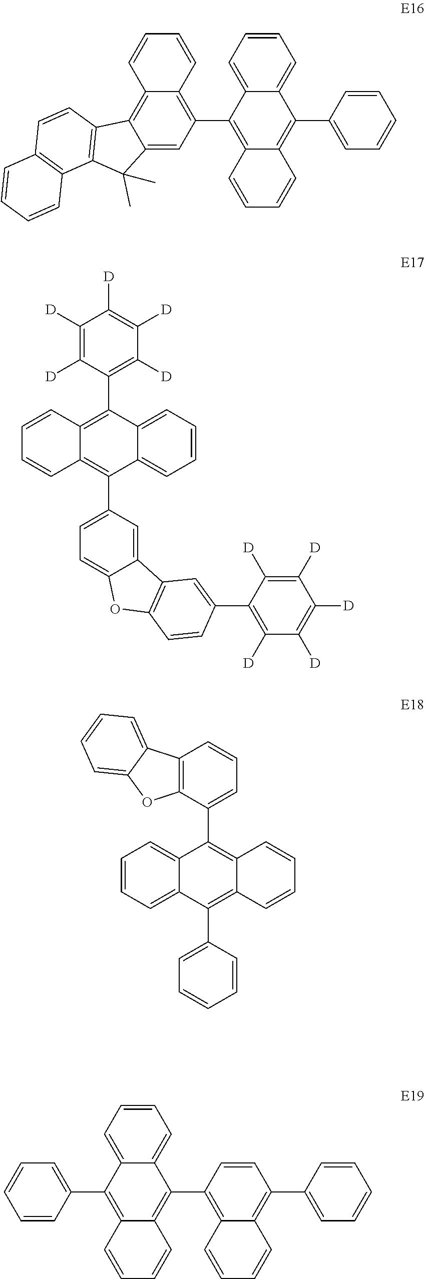

In another embodiment, Formula E-1 above may be represented by any one among the compounds below:

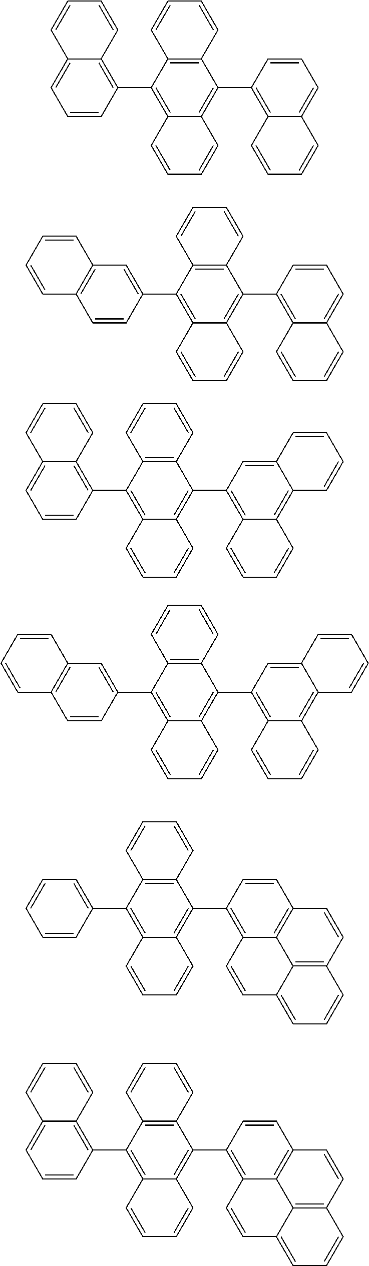

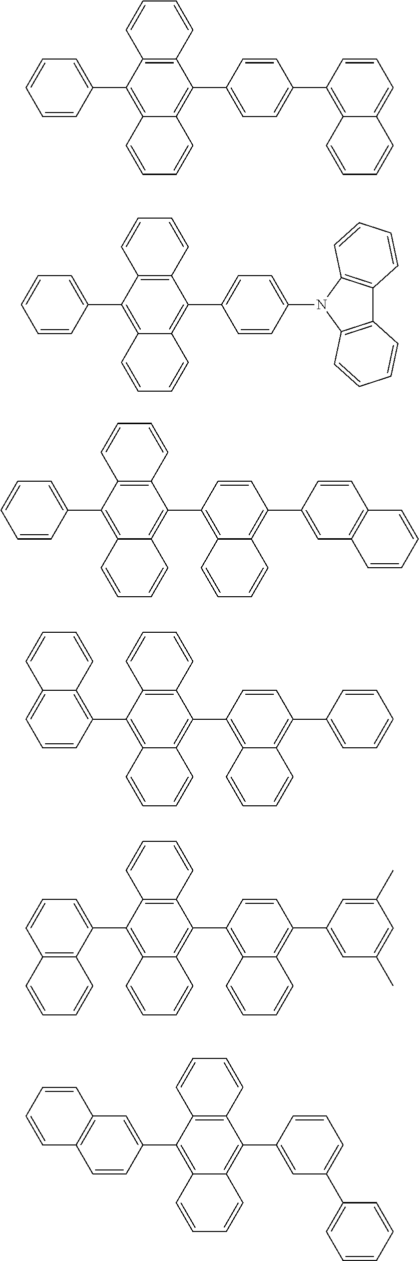

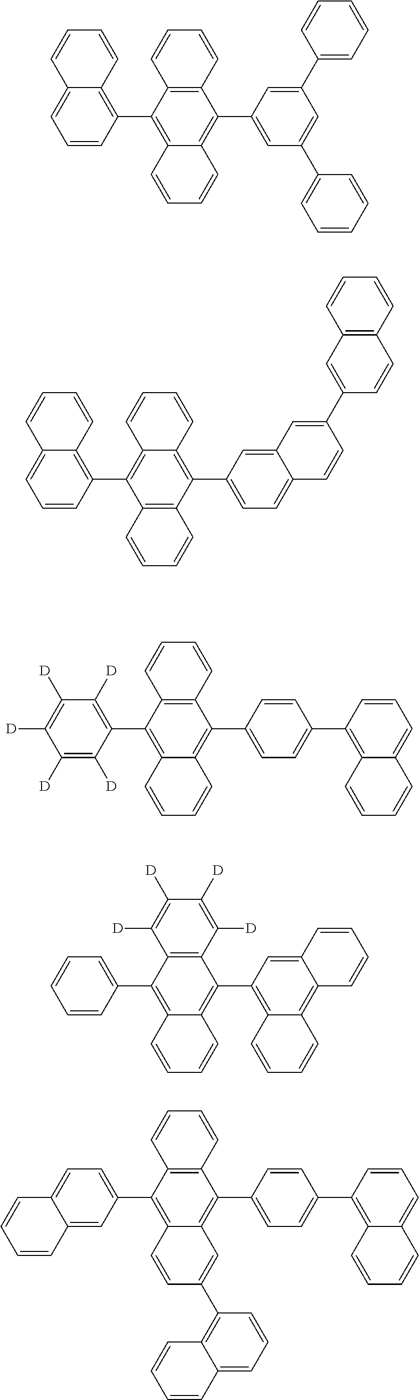

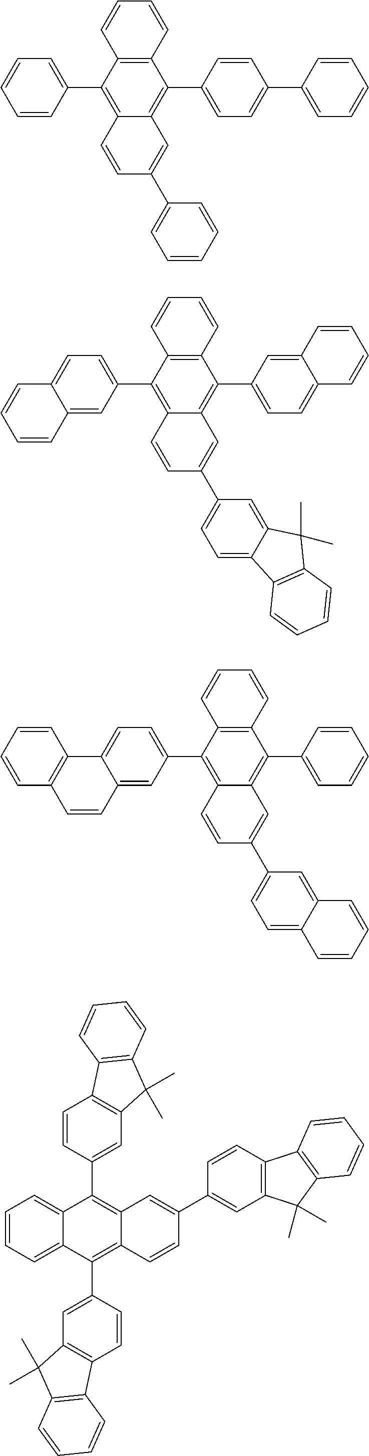

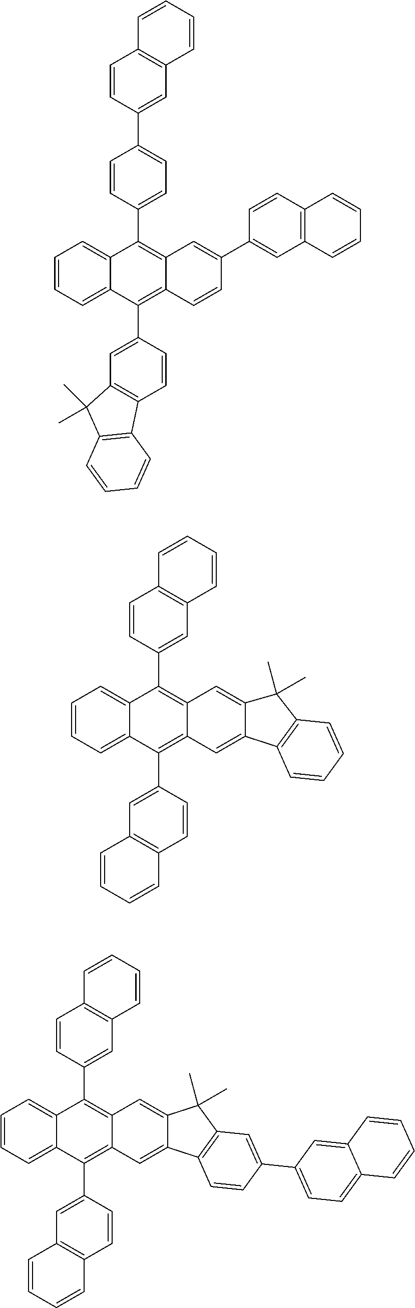

In an embodiment, the emission layer EML may include a compound represented by Formula E-2a or Formula E-2b below. The compound represented by Formula E-2a or Formula E-2b below may be used as a phosphorescence host material.

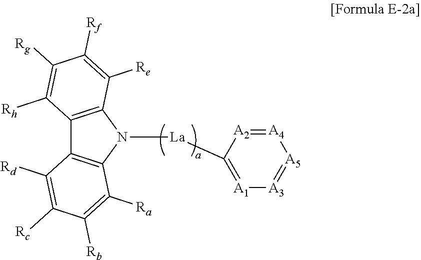

In Formula E-2a, a may be an integer from 0 to 10, and Lₐ may be a direct linkage, a substituted or unsubstituted arylene group having 6 to 30 ring-forming carbon atoms, or a substituted or unsubstituted heteroarylene group having 2 to 30 ring-forming carbon atoms. When a is an integer of 2 or greater, multiple Lₐ(s) may be each independently a substituted or unsubstituted arylene group having 6 to 30 ring-forming carbon atoms, or a substituted or unsubstituted heteroarylene group having 2 to 30 ring-forming carbon atoms.

In Formula E-2a, A₁ to A₅ may each independently be N or C(Rᵢ). Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In an embodiment, Rₐ to Rᵢ may be bonded to an adjacent group to form a hydrocarbon ring or a heterocycle containing N, O, S, etc. as a ring-forming atom.

In Formula E-2a, two or three of A₁ to A₅ may be N, and the remainder of A₁ to A₅ may be C(Rᵢ).

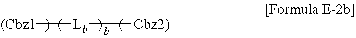

In Formula E-2b, Cbz1 and Cbz2 may each independently be an unsubstituted carbazole group, or a carbazole group substituted with an aryl group having 6 to 30 ring-forming carbon atoms. L_{b} may be a direct linkage, a substituted or unsubstituted arylene group having 6 to 30 carbon atoms for forming a ring, or a substituted or unsubstituted heteroarylene group having 2 to 30 carbon atoms for forming a ring. In Formula E-2b, b may be an integer from 0 to 10, and when b is an integer of 2 or more, multiple L_{b}(s) may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among the compounds of Compound Group E-2 below. However, the compounds listed in Compound Group E-2 below are examples, the compound represented by Formula E-2a or Formula E-2b is not limited to those represented by Compound Group E-2 below.

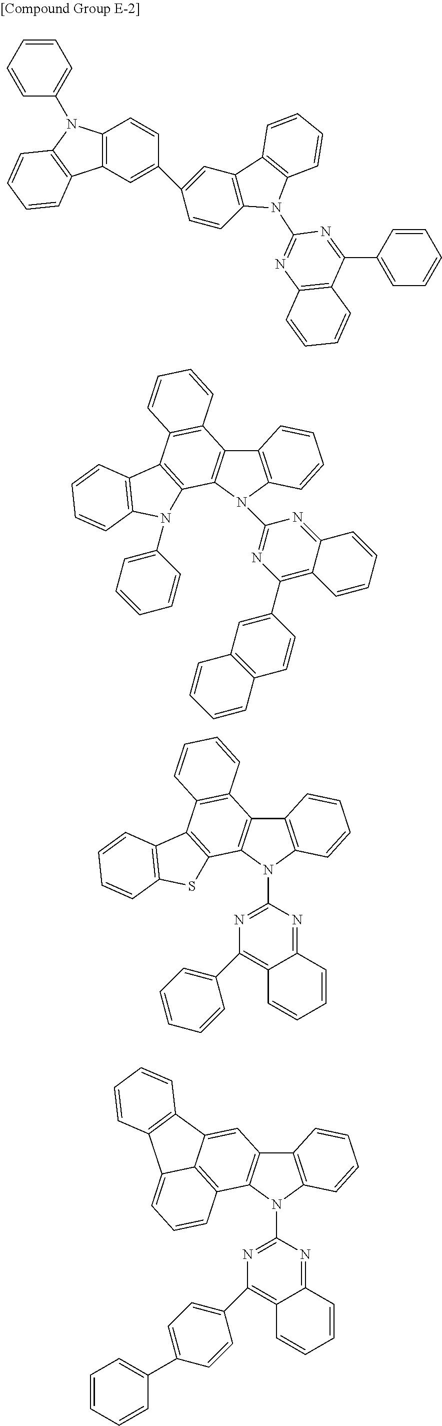

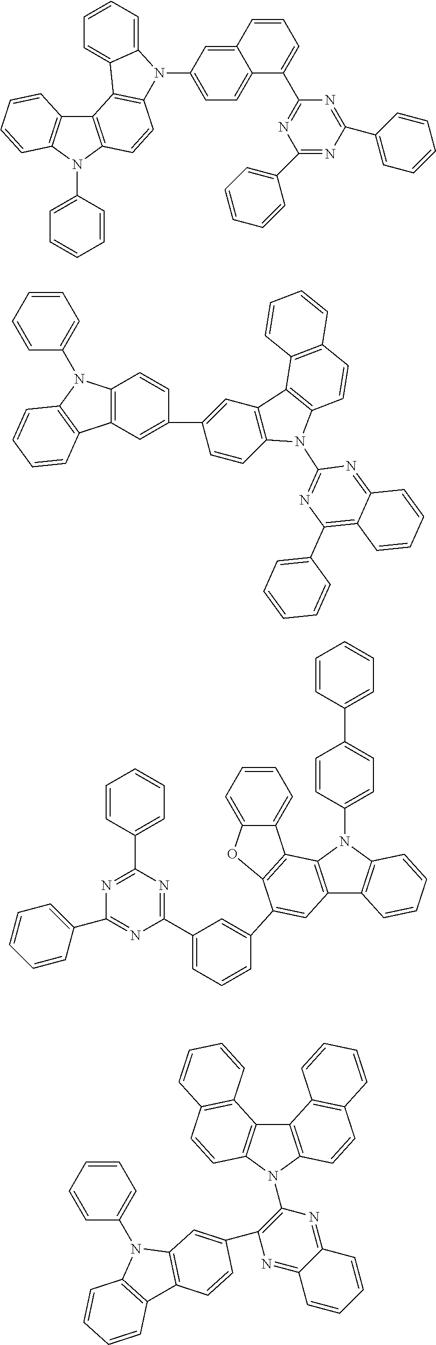

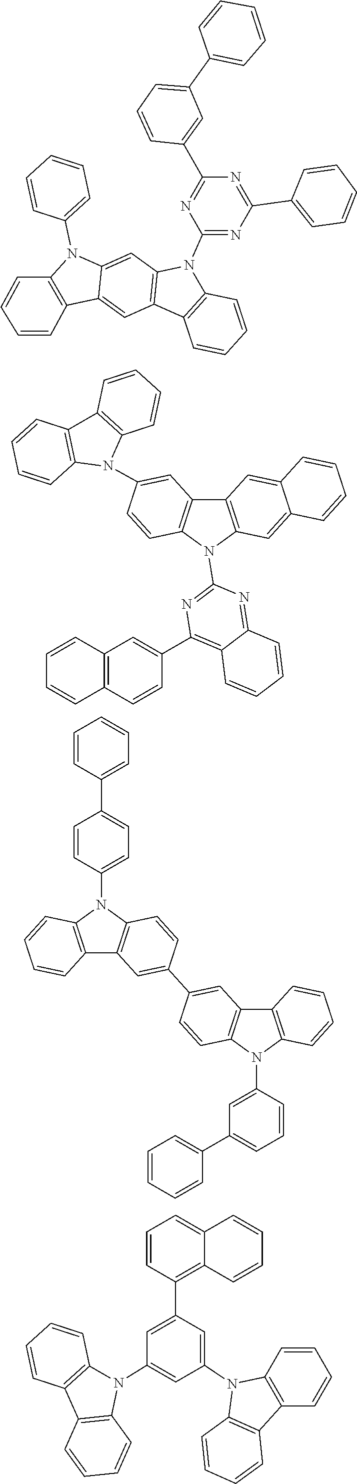

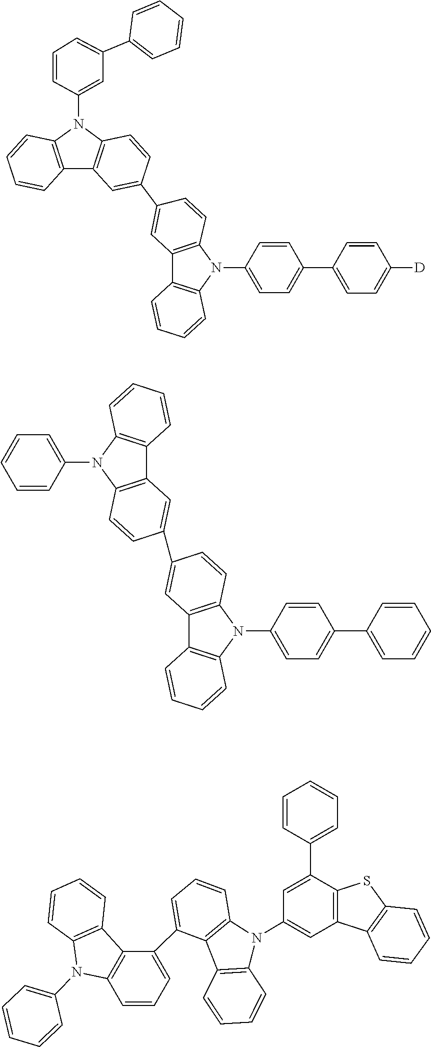 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

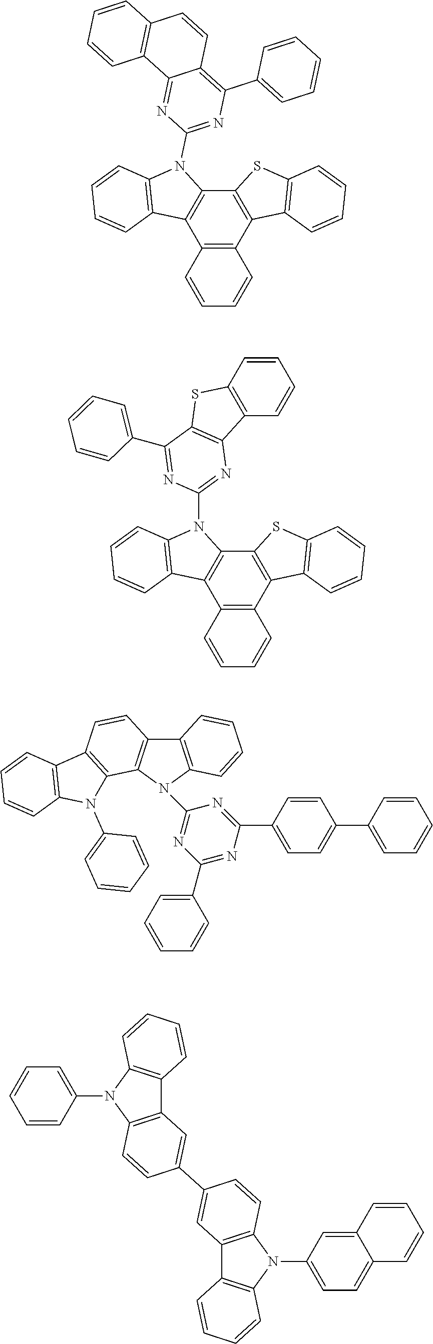 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

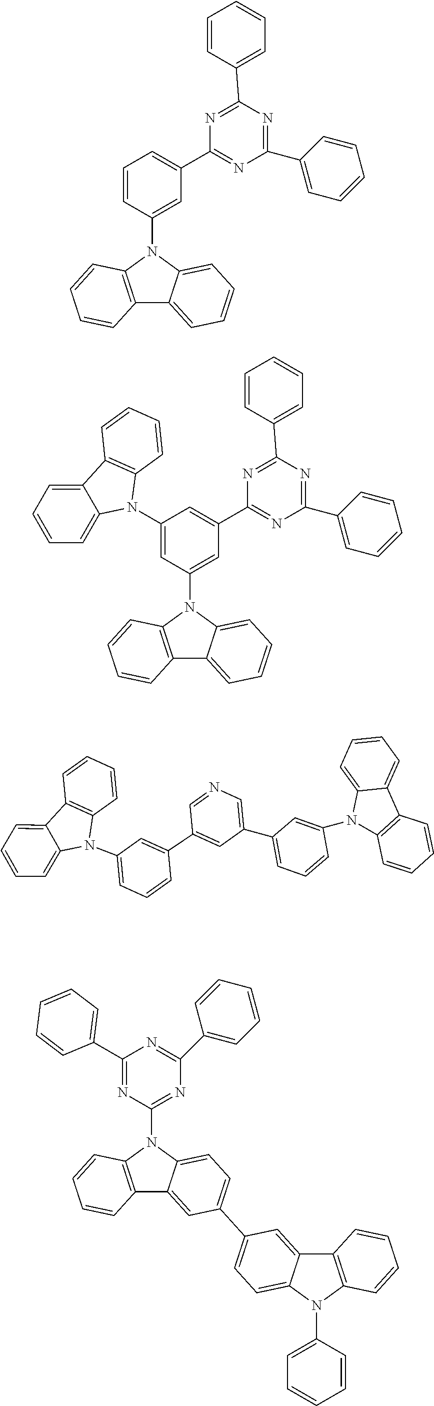 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

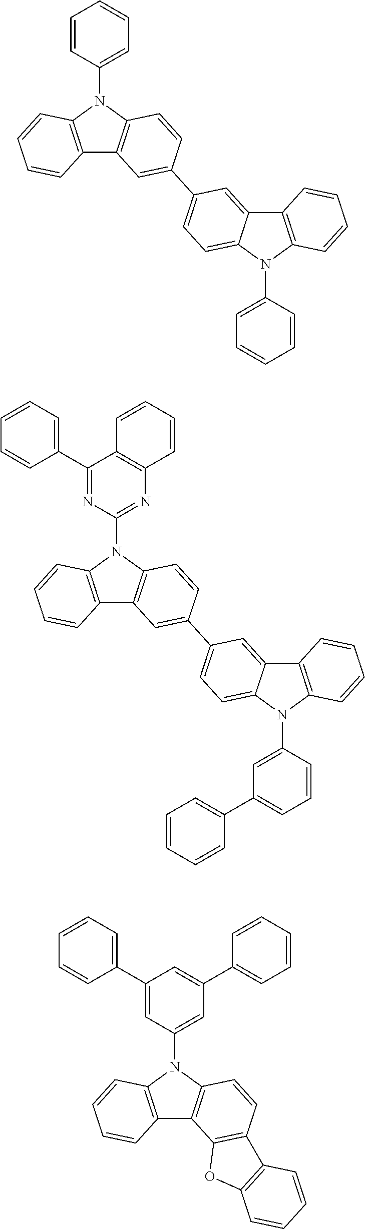 [SMILES: c1ccc(-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

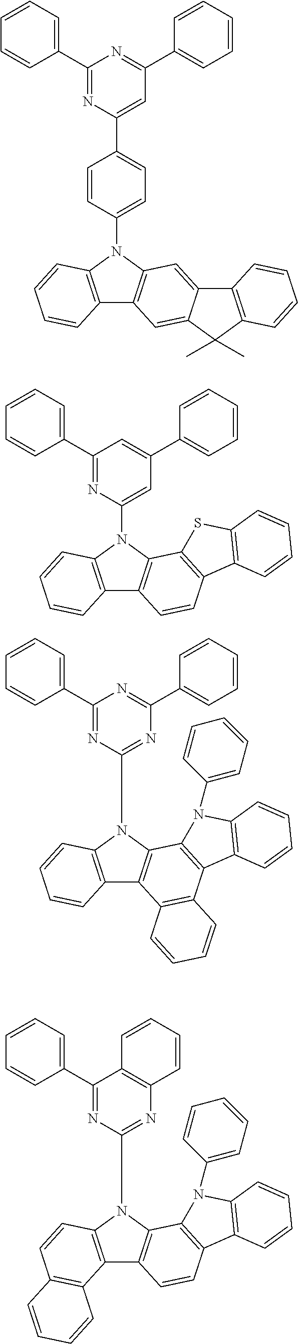

The emission layer EML may further include a general material in the art as a host material. For example, the emission layer EMVL may include, as a host material, at least one of bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments are not limited thereto, and for example, tris(8-hydroxyquinolino)aluminum (Alq₃), 4,4′-bis(N-carbazolyl)-1,1′-biphenyl (CBP), poly(n-vinylcabazole (PVK), 9,10-di(naphthalene-2-yl)anthracene (ADN), 4,4′,4″-Tris(carbazol-9-yl)-triphenylamine (TCTA), 1,3,5-tris(N-phenylbenzimidazole-2-yl)benzene (TPBi), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenylcyclotriphosphazene (CP1), 1,4-bis(triphenylsilyl)benzene (UGH2), hexaphenylcyclotrisiloxane (DPSiO₃), octaphenylcyclotetra siloxane (DPSiO₄), 2,8-bis(diphenylphosphoryl)dibenzofuran (PPF), etc. may be used as a host material.

The emission layer EML may include a compound represented by Formula M-a or Formula M-b below. The compound represented by Formula M-a or Formula M-b below may be used as a phosphorescence dopant material.

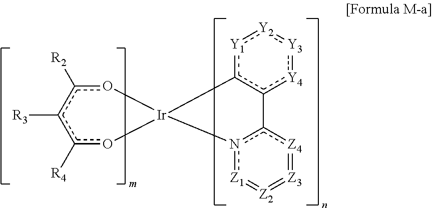

In Formula M-a above, Y₁ to Y₄ and Z₁ to Z₄ may each independently be C(R₁) or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m may be 0 or 1, and n may be 2 or 3. In Formula M-a, when m is 0, n may be 3, and when m is 1, n may be 2.

The compound represented by Formula M-a may be used as a red phosphorescence dopant or a green phosphorescence dopant.

The compound represented by Formula M-a may be represented by any one among Compound M-a1 to Compound M-a23 below. However, Compounds M-a1 to M-a23 below are examples, and the compound represented by Formula M-a is not limited to those represented by Compounds M-a1 to M-a23 below.

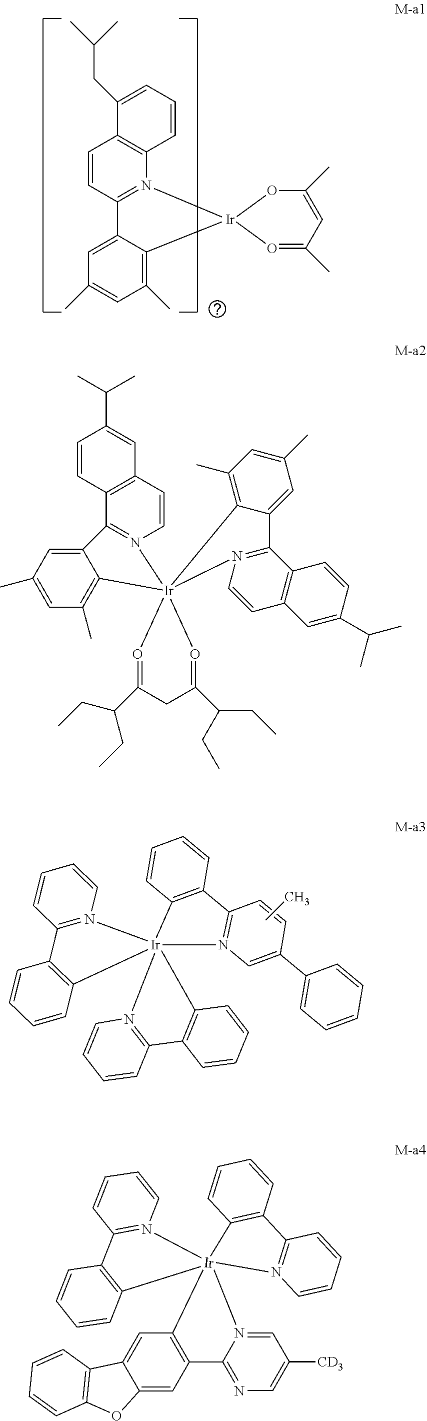

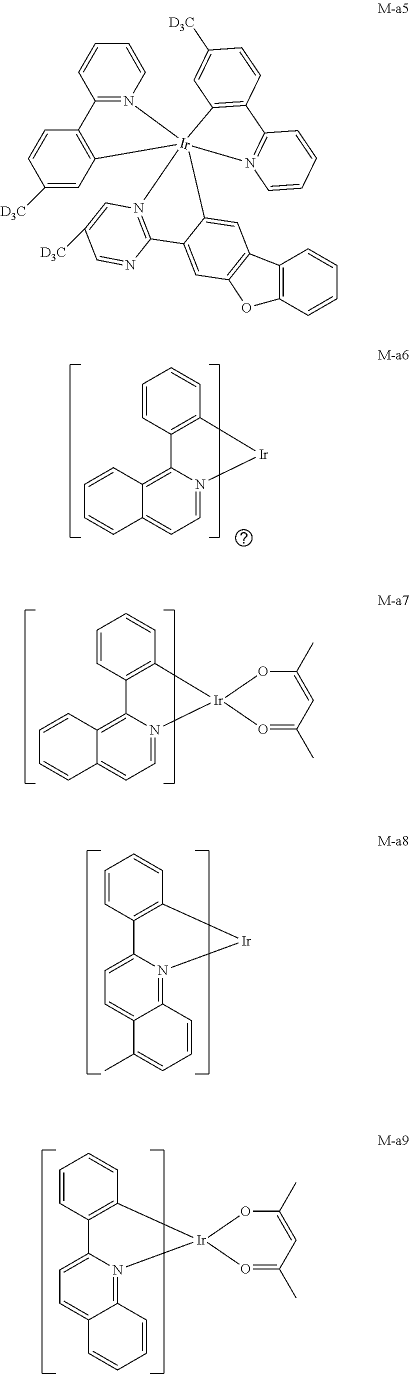

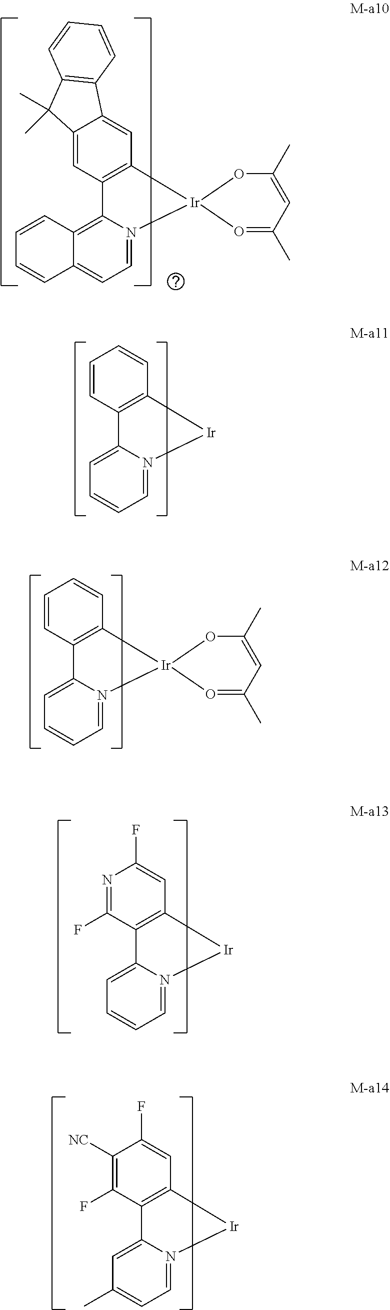

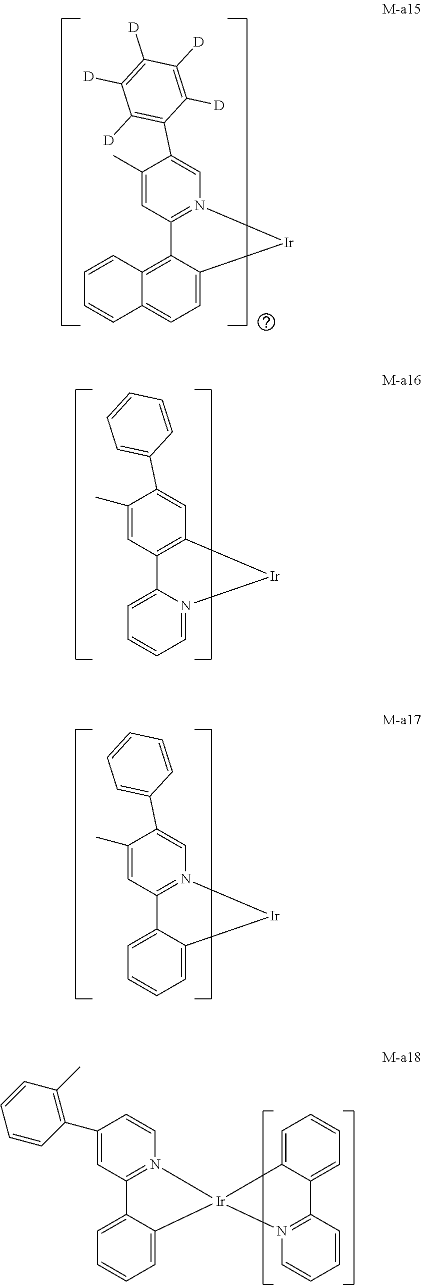

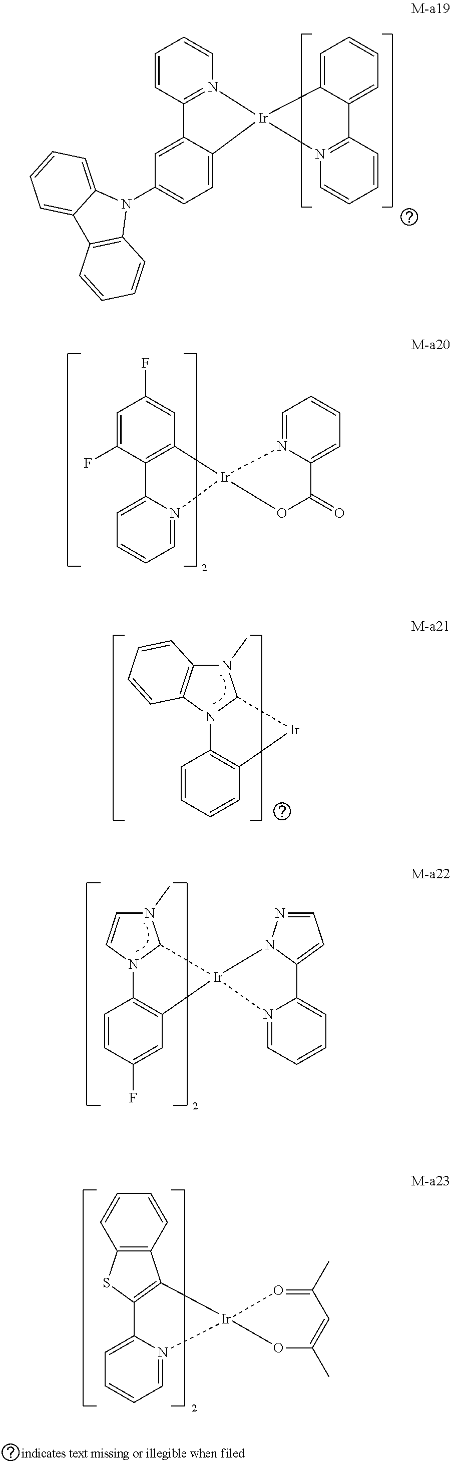

Compound M-a1 and Compound M-a2 may be used as a red dopant material, and Compound M-a3 to Compound M-a5 may be used as a green dopant material.

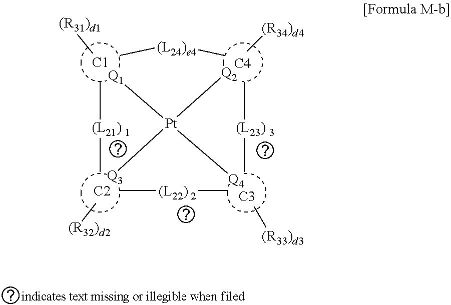

In Formula M-b, Qi to Q4 may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L21 to L24 may each independently be a direct linkage,

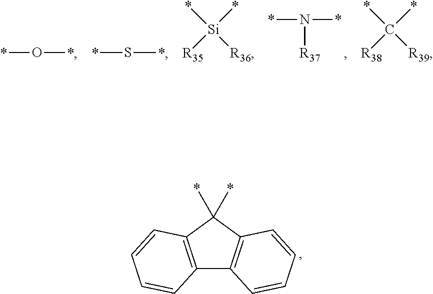

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring, and d1 to d4 may each independently be an integer from 0 to 4.

The compound represented by Formula M-b may be used as a blue phosphorescence dopant or a green phosphorescence dopant.

The compound represented by Formula M-b may be represented by any one among the compounds below. However, the compounds below are examples, and the compound represented by Formula M-h is not limited to those represented by the compounds below.

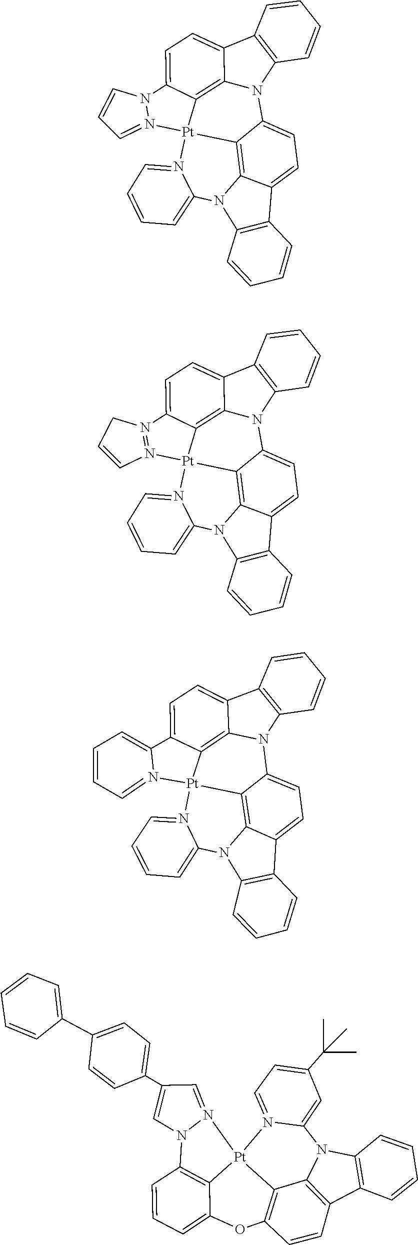

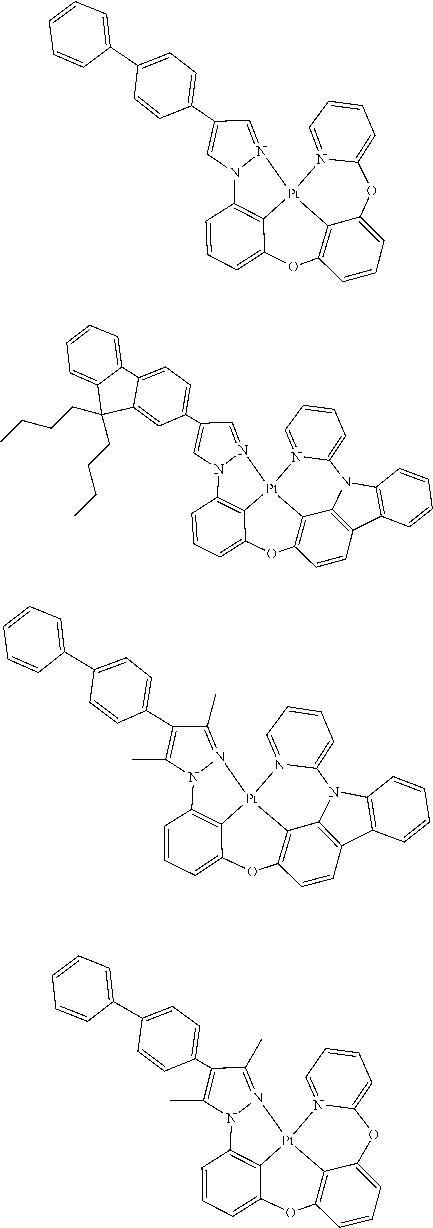

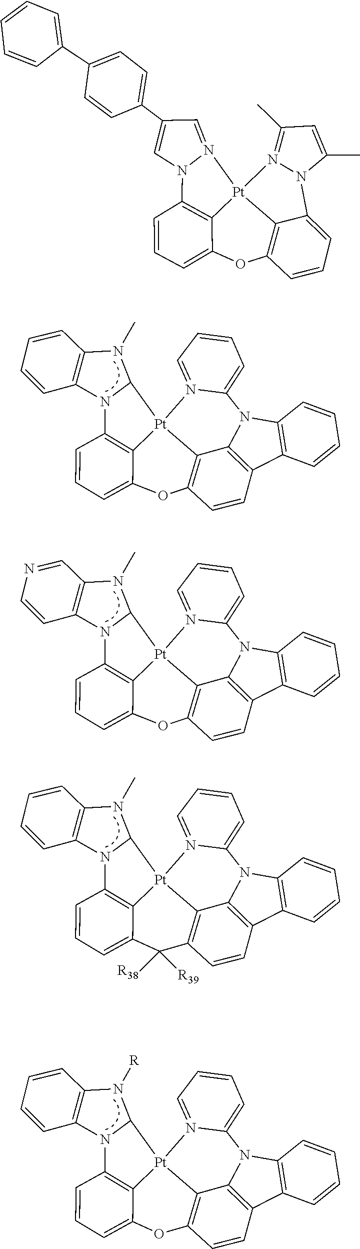

In the compounds, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

At least one among the compounds below including Pt as a central metal may be included as a dopant.

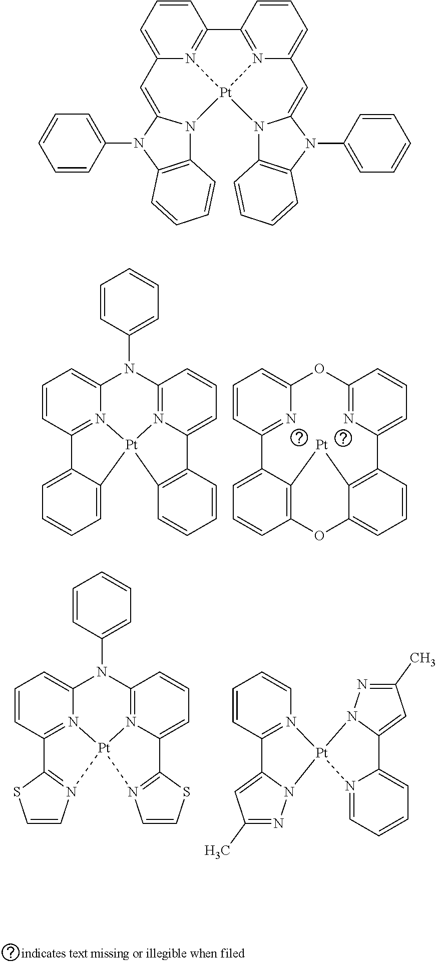

The emission layer EML may include a compound represented by any one among Formula F-a to Formula F-c below. The compound represented by Formula F-a or Formula F-c below may be used as a fluorescence dopant material.

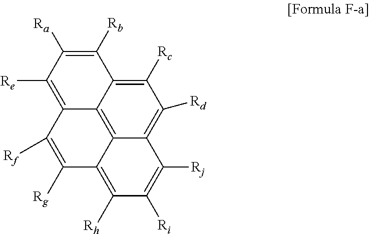

In Formula F-a, two selected from among Rₐ to Rⱼ may each independently be substituted with

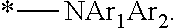

The remainder of Rₐ to Rⱼ, which are not substituted with

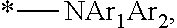

may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In

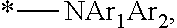

Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ and Ar₂ may be a heteroaryl group containing O or S as a ring-forming atom.

The emission layer may include, as a fluorescence dopant, at least one among Compound FD1 to Compound FD22 below.

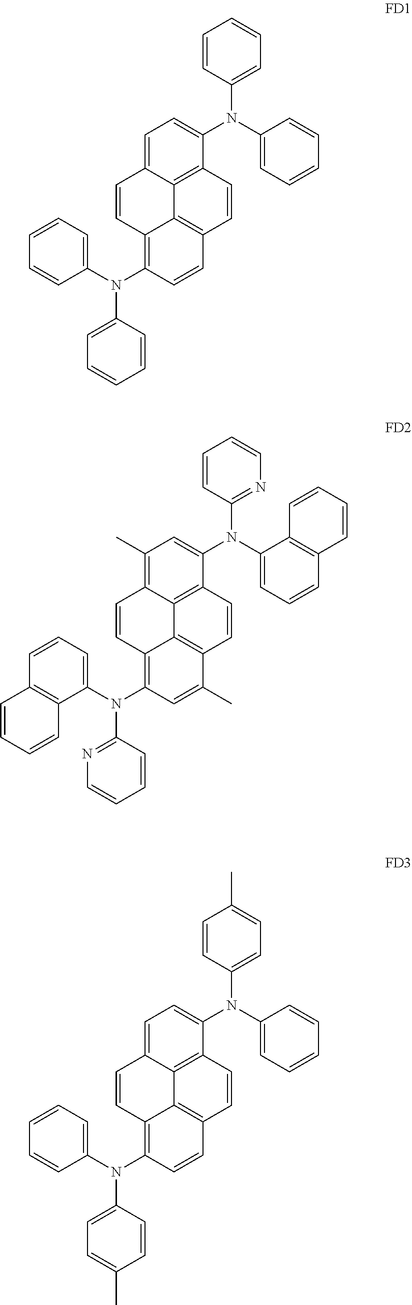 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cc5)ccc5ccc2c3c54)cc1]

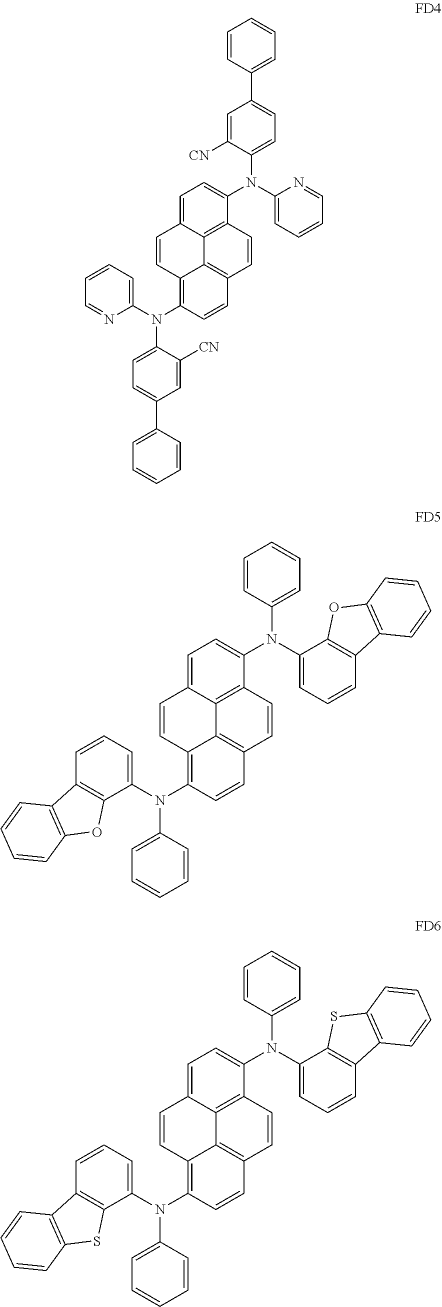

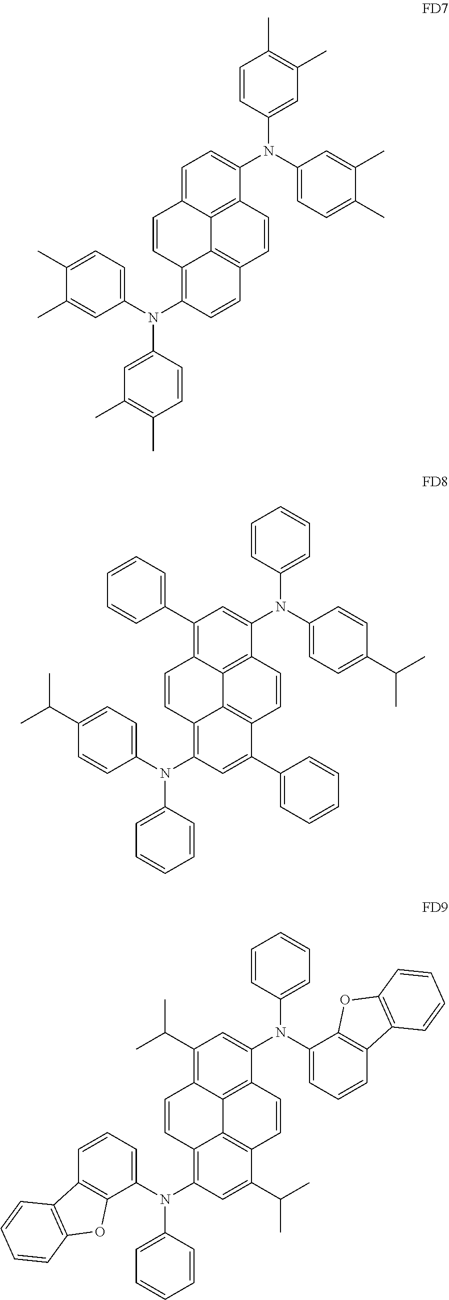

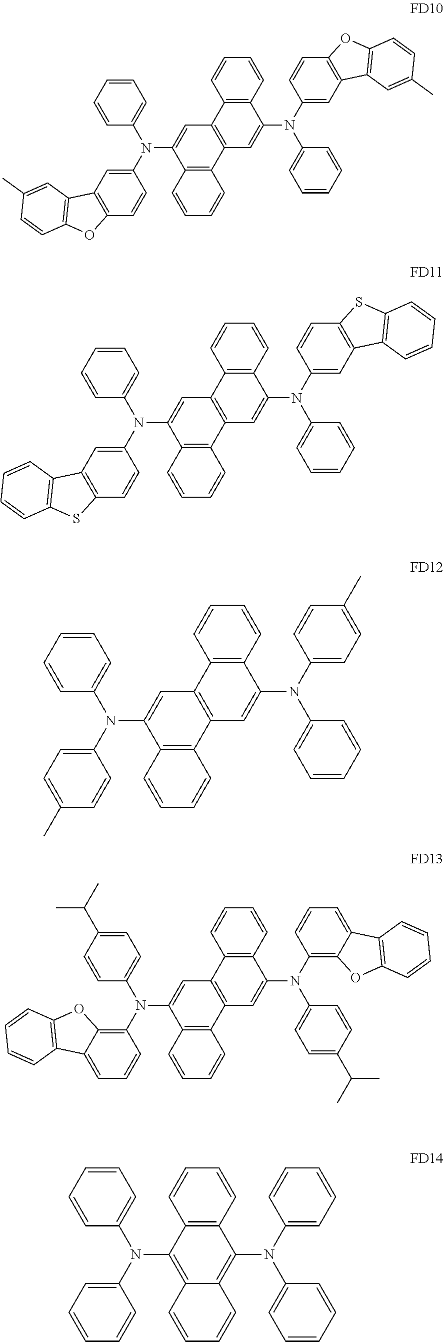

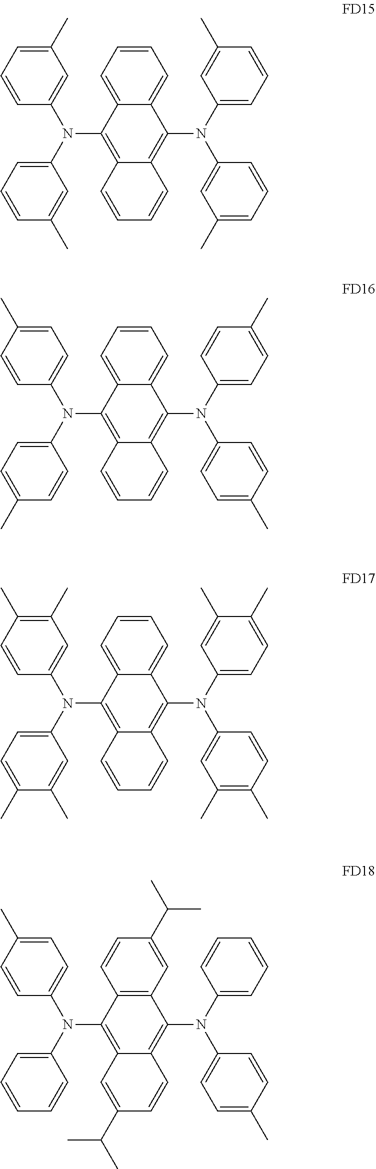 [SMILES: Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

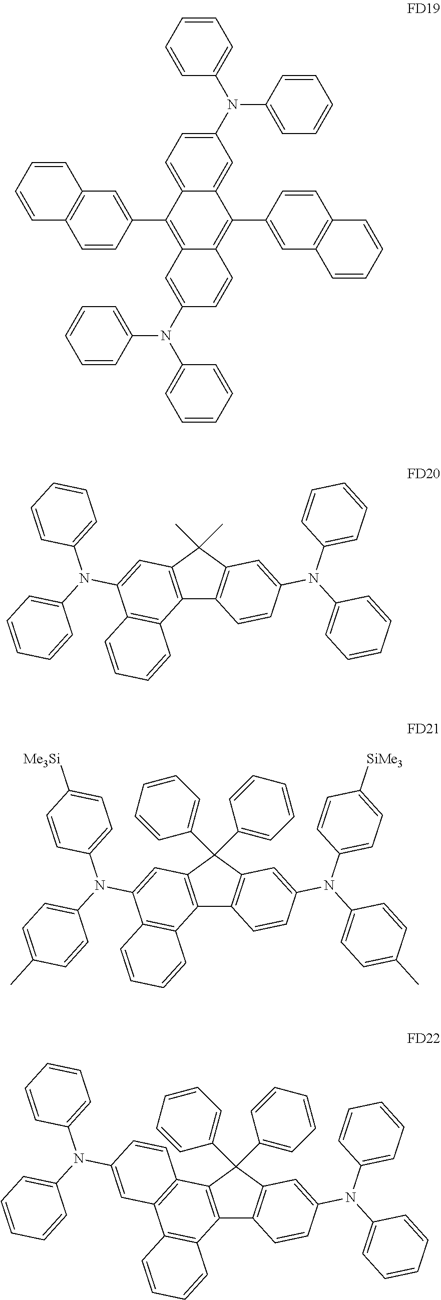

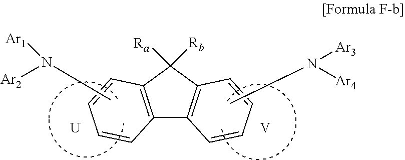

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, when the number of U or V is 1, a ring may form a condensed ring at a part described as U or V, and when the number of U or V is 0, a ring described as U or V may not be present. For example, when the number of U is 0 and the number of V is 1, or when the number of U is 1 and the number of V is 0, the condensed ring having a fluorene core of Formula F-b may be a four-ring cyclic compound. When the number of U and V is each 0, the condensed ring of Formula F-b may be a three-ring cyclic compound. When the number of U and V is each 1, the condensed ring having a fluorene core of Formula F-b may be a five-ring cyclic compound.

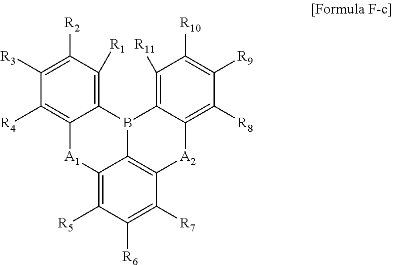

In Formula F-c, A₁ and A₂ may each independently be O, S, Se, or N(Rₘ), and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-c, A₁ and A₂ may each independently be bonded to substituents of an adjacent ring to form a condensed ring. For example, when A₁ and A₂ are each independently N(Rₘ), A₁ may be bonded to R₄ or R₅ to form a ring. In an embodiment, in Formula F-c, A₂ may be bonded to R₇ or R₈ to form a ring.

In an embodiment, the emission layer EML may include, as a dopant material, styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenami ne (N-BDAVBi), 4,4′-bis[2-(4-(N,N-diphenylamino)phenyl)vinyl]biphenyl(DPAVBi), perylene and the derivatives thereof (e.g., 2,5,8,11-tetra-t-butylperylene (TBP)), pyrene and the derivatives thereof (e.g., 1,1-dipyrene, 1,4-dipyrenylbenzene, 1,4-bis(N,N-diphenylamino)pyrene), etc.

The emission layer EML may include a phosphorescence dopant material. For example, a metal complex including iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescence dopant. For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as a phosphorescence dopant. However, embodiments are not limited thereto.

The emission layer EML may include a quantum dot material. The core of the quantum dot may be selected from among a Group II-VI compound, a Group III-VI compound, a Group I-III-VI compound, a Group III-V compound, a Group III-II-V compound, a Group IV-VI compound, a Group IV element, a Group IV compound, and a combination thereof.

A Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ and In₂Se₃, a ternary compound such as InGaS₃ and InGaSe₃, or any combination thereof.

A Group I-III-VI compound may be selected from a ternary compound selected from the group consisting of AgInS, AgInS₂, CuInS, CuInS₂, AgGaS₂, CuGaS₂ CuGaO₂, AgGaO₂, AgAlO₂, and a mixture thereof, or a quaternary compound such as AgInGaS₂ and CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. The Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

For example, a binary compound, a ternary compound, or a quaternary compound may be present in particles in a uniform concentration distribution, or may be present in the same particle in a partially different concentration distribution. The quantum dot may have a core/shell structure in which one quantum dot surrounds another quantum dot. An interface between the core and the shell may have a concentration gradient in which the concentration of an element present in the shell becomes lower toward the center.

In embodiments, a quantum dot may have the above-described core-shell structure including a core having nanocrystals and a shell surrounding the core. The shell of the quantum dot may serve as a protection layer to prevent the chemical deformation of the core so as to maintain semiconductor properties, and/or a charging layer to impart electrophoresis properties to the quantum dot. The shell may be a single layer or multiple layers. An interface between the core and the shell may have a concentration gradient in which the concentration of an element present in the shell becomes lower toward the center. An example of the shell of the quantum dot may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and NiO, or a ternary compound such as MgAl₂O₄, CoFe₂O₄, NiFe₂O₄, and CoMn₂O₄, but embodiments are not limited thereto.

In an embodiment, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width of half maximum (FWHM) of a light emission wavelength spectrum equal to or less about 45 nm. For example, the quantum dot may have a FWHM of a light emission wavelength spectrum equal to or less than about 40 nm. For example, the quantum dot may have a FWHM of a light emission wavelength spectrum equal to or less than about 30 nm. Color purity or color reproducibility may be improved in the above-described ranges. Light emitted through such a quantum dot may be emitted in all directions, and thus a wide viewing angle may be improved.

The form of a quantum dot is not particularly limited. For example, a spherical, a pyramidal, a multi-arm, or a cubic quantum dot may be used, or a quantum dot in the form of nanoparticles, nanotubes, nanowires, nanofibers, nanoparticles, etc. may be used.

A quantum dot may control the color of emitted light according to the particle size thereof and thus the quantum dot may have various light emission colors such as green, red, etc.

In each light emitting device ED of embodiments illustrated in FIGS. 3 to 6, the electron transport region ETR is provided on the emission layer EML. The electron transport region ETR may include at least one of the hole blocking layer HBL, the electron transport layer ETL, and the electron injection layer EIL, but embodiments are not limited thereto.

The electron transport region ETR may have a single layer formed of a single material, a single layer formed of different materials, or a multilayer structure including multiple layers formed of different materials.

For example, the electron transport region ETR may have a single layer structure of the electron injection layer EIL or the electron transport layer ETL, and may have a single layer structure formed of an electron injection material and an electron transport material. The electron transport region ETR may have a single layer structure formed of different materials, or may have a structure in which an electron transport layer ETL/electron injection layer EIL and a hole blocking layer HBL/electron transport layer ETL/electron injection layer EIL are stacked in order from the emission layer EML, but embodiments are not limited thereto. A thickness of the electron transport region ETR may be, for example, in a range of about 1000 Å to about 1,500 Å.

The electron transport region ETR may be formed by using various methods such as a vacuum deposition method, a spin coating method, a cast method, a Langmuir-Blodgett (LB) method, an inkjet printing method, a laser printing method, a laser induced thermal imaging (LITI) method, etc.

The electron transport region ETR may include a compound represented by Formula ET-1 below:

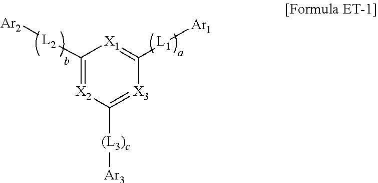

In Formula ET-1, at least one of X₁ to X₃ is N, and the remainder of X₁ to X₃ may be C(Rₐ). Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula ET-1, when a to c are an integer of 2 or greater, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebg₂), 9,10-di(naphthalene-2-yl)anthracene (ADN), 1,3-Bis[3,5-di(pyridin-3-yl)phenyl]benzene (BmPyPhB), or a mixture thereof.

The electron transport region ETR may include at least one among Compound ET1 to Compound ET36 below:

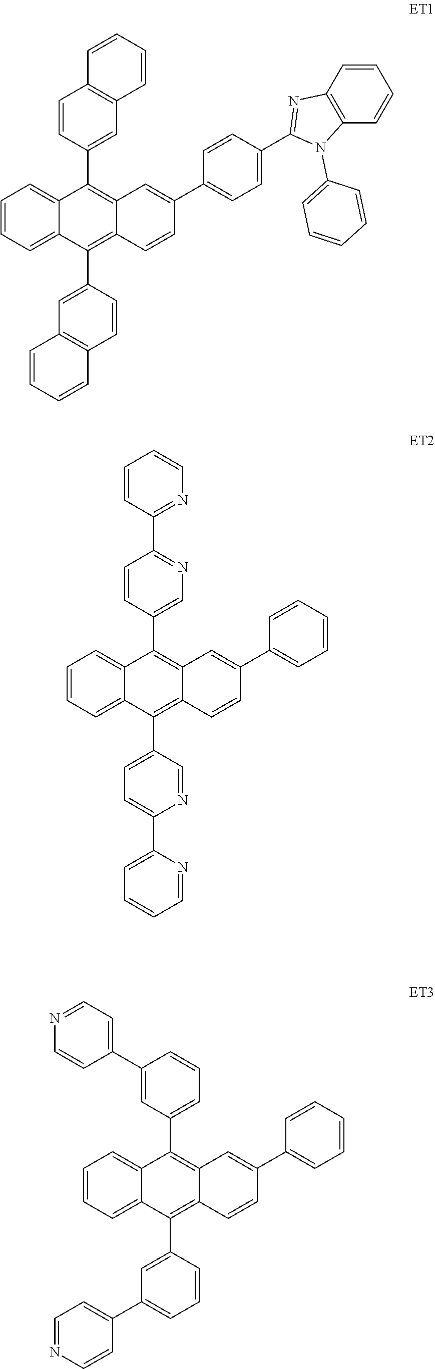 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

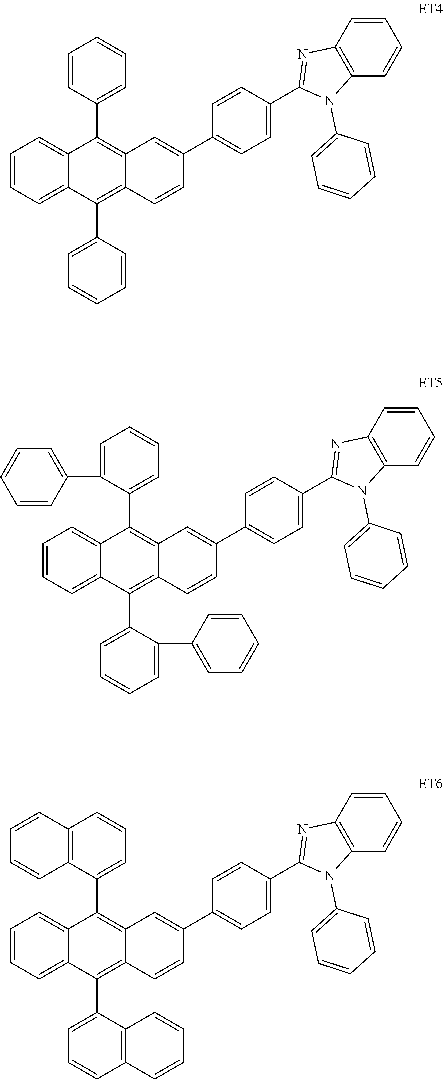 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

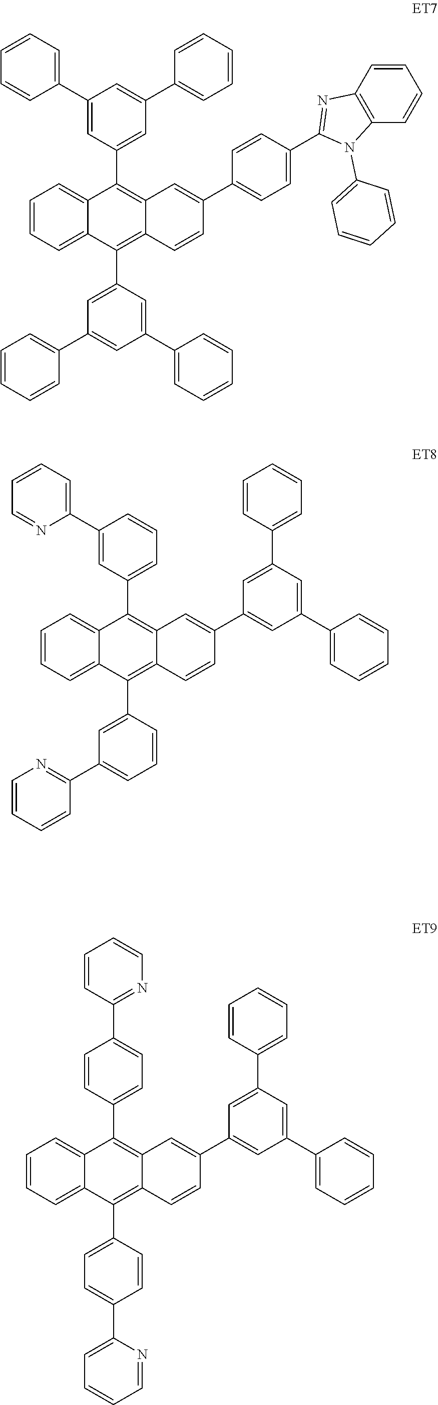 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

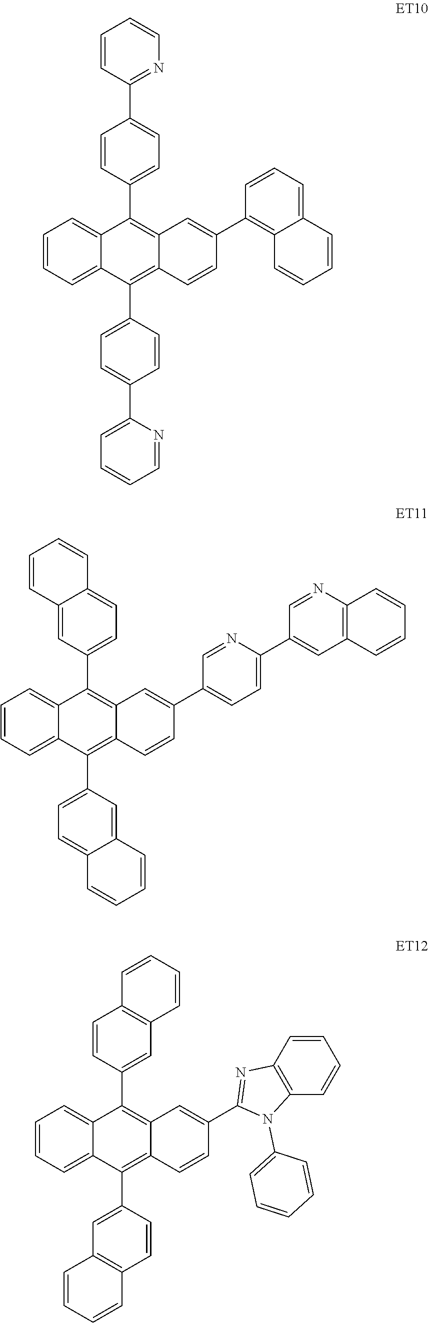 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

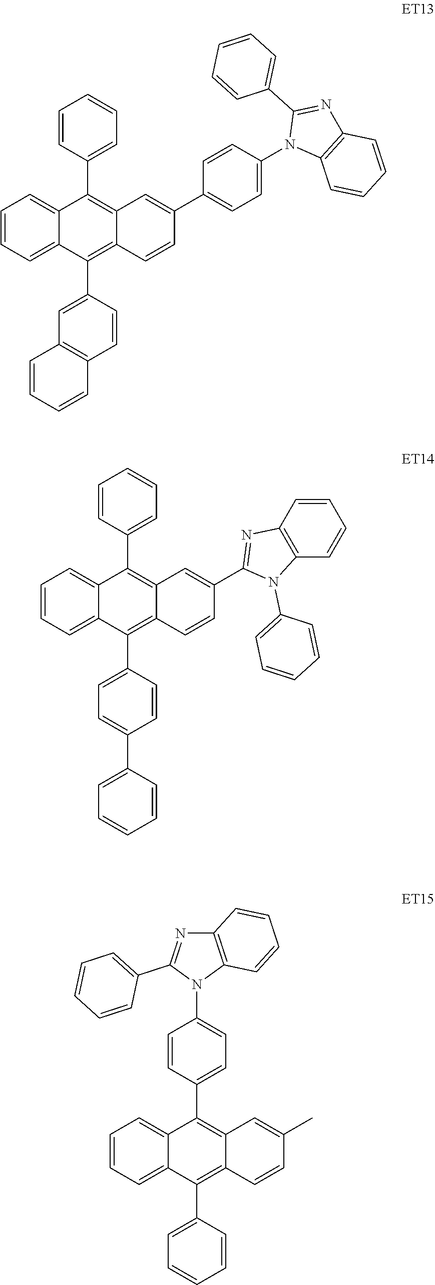 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

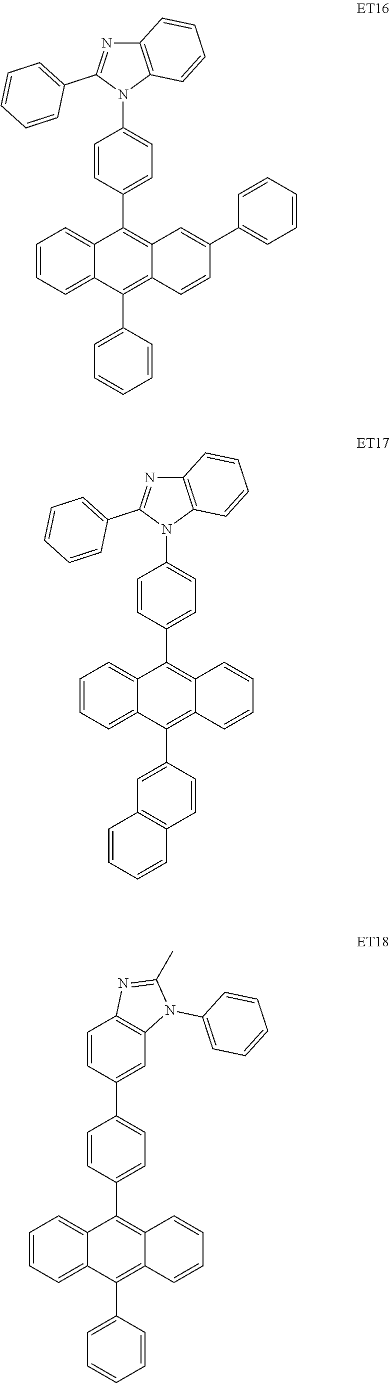 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

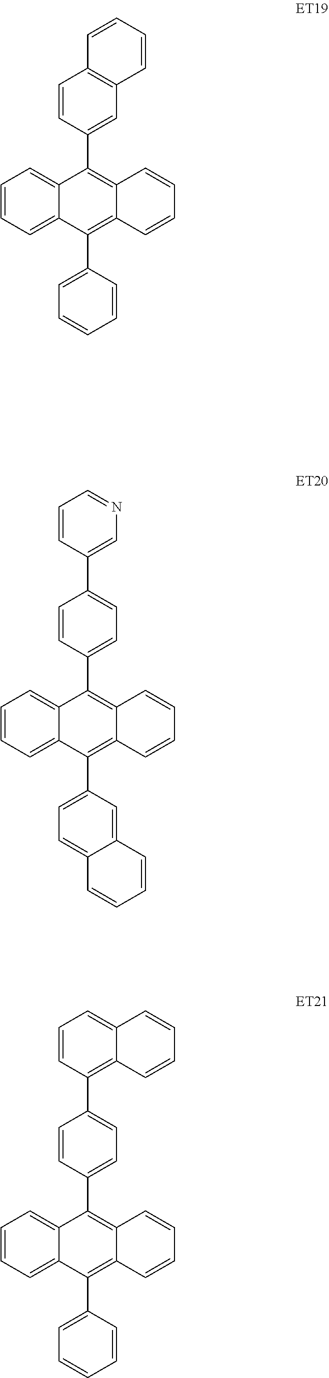

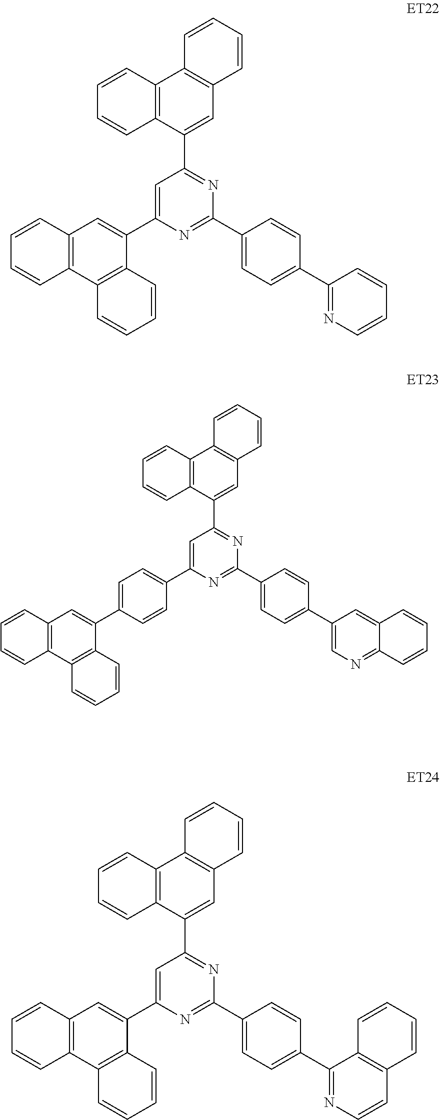 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

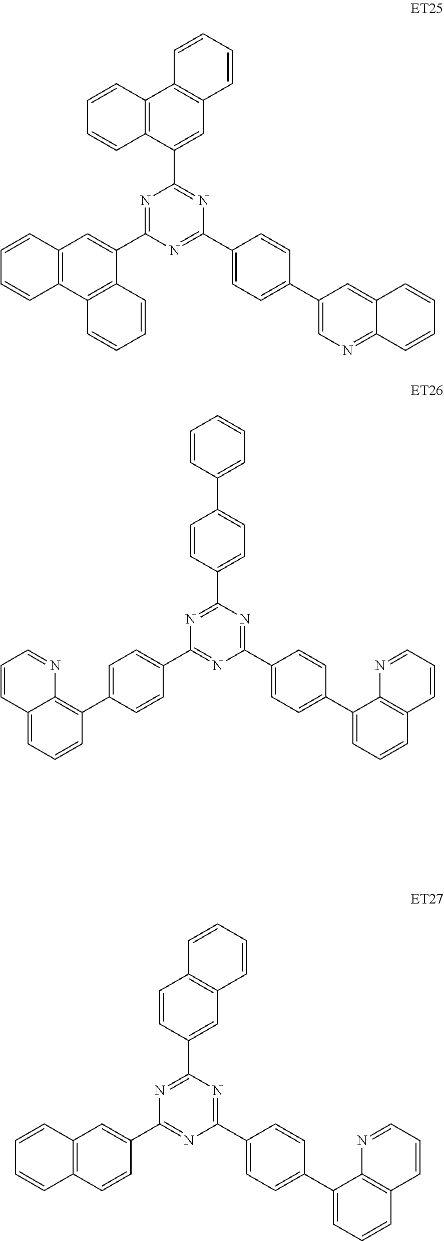 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

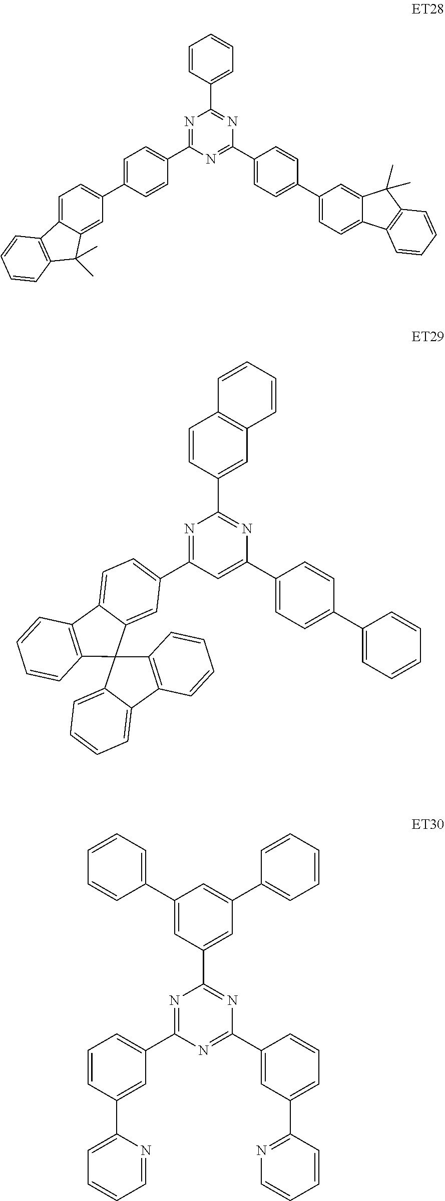 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

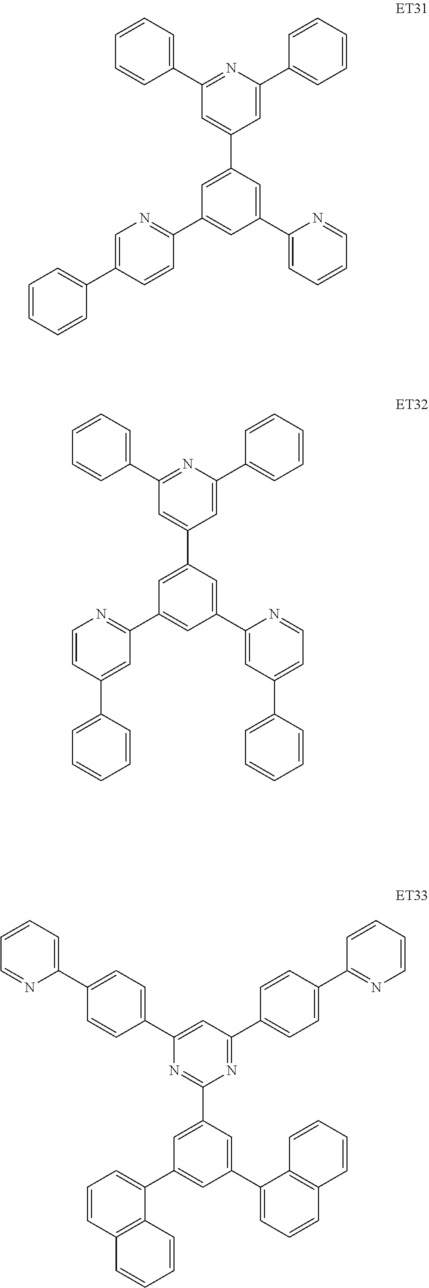 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

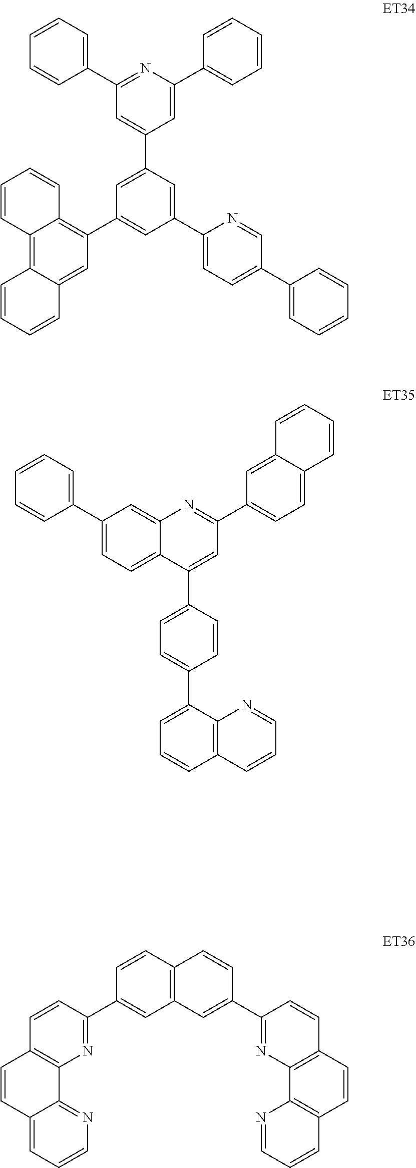 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

The electron transport regions ETR may include a metal halide such as LiF, NaCl, CsF, RbCl, RbI, CuI, or KI, a lanthanide metal such as Yb, and a co-deposited material of the metal halide and the lanthanide metal. For example, the electron transport region ETR may include KI:Yb, RbI:Yb, etc. as a co-deposited material. The electron transport region ETR may be formed using a metal oxide such as Li₂O or BaO, or 8-hydroxyl-lithium quinolate (Liq), etc., but embodiments are not limited thereto. The electron transport region ETR may also be formed of a mixture material of an electron transport material and an insulating organometallic salt. The organometallic salt may be a material having an energy band gap equal to or greater than about 4 eV. For example, the organometallic salt may include metal acetates, metal benzoates, metal acetoacetates, metal acetylacetonates, or metal stearates.

The electron transport region ETR may further include at least one of 2,9-dimethyl-4,7-diphenyl-1,10-phenanthroline (BCP) and 4,7-diphenyl-1,10-phenanthroline (Bphen) in addition to the above-described materials, but embodiments are not limited thereto.

The electron transport region ETR may include the above-described compounds of the electron transport region in at least one of the electron injection layer EIL, the electron transport layer ETL, and the hole blocking layer HBL.

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. If the thickness of the electron transport layer ETL satisfies the aforementioned ranges, satisfactory electron transport characteristics may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. If the thickness of the electron injection layer EIL satisfies the above-described ranges, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgYb). In other embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, or the like.

Although not shown, the second electrode EL2 may be connected with an auxiliary electrode. If the second electrode EL2 is connected with an auxiliary electrode, the resistance of the second electrode EL2 may decrease.

A capping layer CPL may be further disposed on the second electrode EL2 of the light emitting device ED of an embodiment. The capping layer CPL may include a multilayer or a single layer.

In an embodiment, the capping layer CPL may be an organic layer or an inorganic layer. For example, when the capping layer CPL includes an inorganic material, the inorganic material may include an alkaline metal compound such as LiF, an alkaline earth metal compound such as MgF₂, SiON, SiNₓ, SiO_{y}, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or an epoxy resin, or acrylate such as methacrylate. However, embodiments are not limited thereto, and the capping layer CPL may include at least one among Compounds P1 to P5 below.

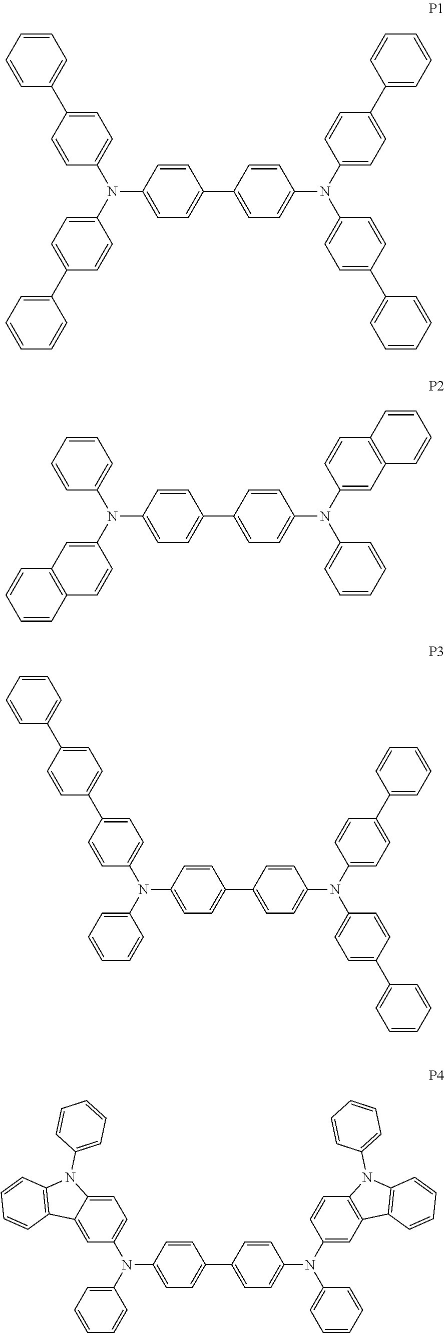 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

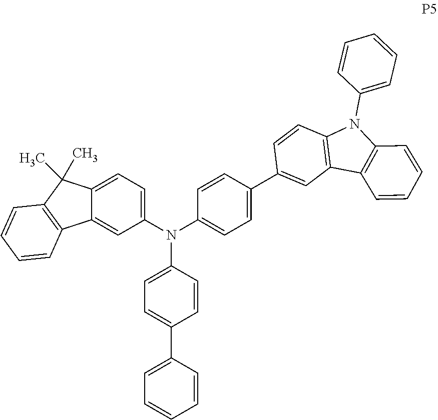 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6 with respect to light in a wavelength range of about 550 nm to about 660 nm.

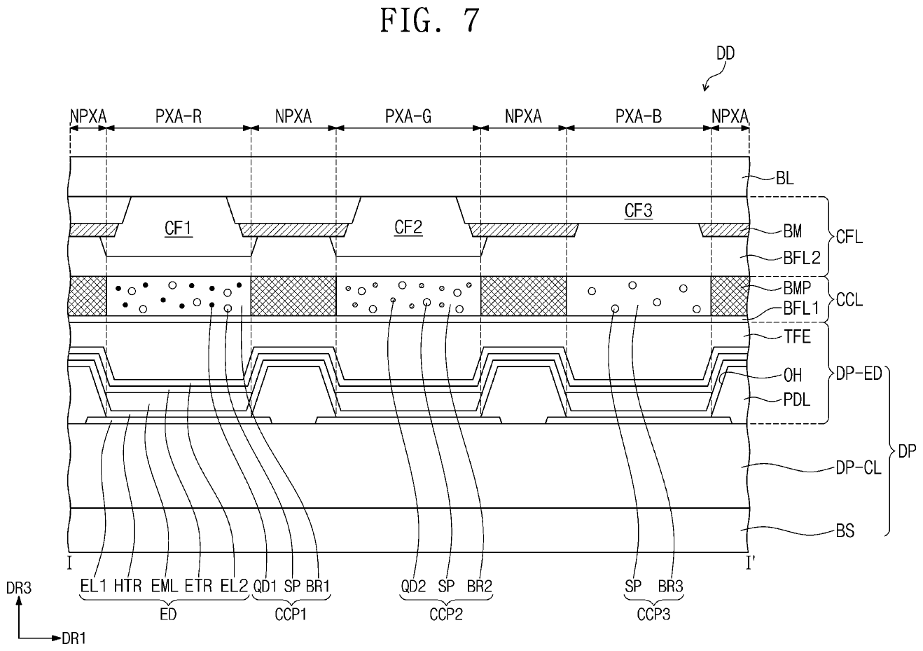

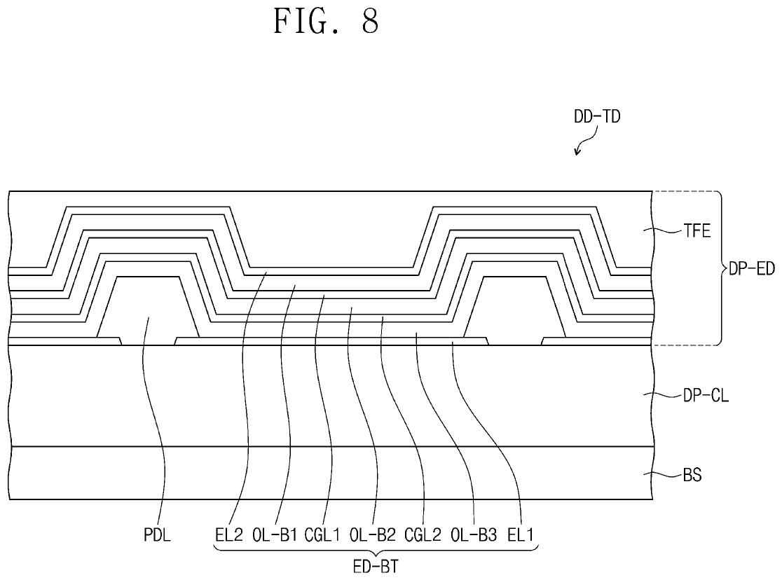

FIGS. 7 and 8 each are a schematic cross-sectional view of a display apparatus according to an embodiment. Hereinafter, in describing the display apparatus of an embodiment with reference to FIGS. 7 and 8, the duplicated features which have been described in FIGS. 1 to 6 are not described again, but their differences will be described.

Referring to FIG. 7, the display apparatus DD according to an embodiment may include a display panel DP including a display device layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The structures of the light emitting devices of FIGS. 3 to 6 as described above may be applied to the structure of the light emitting device ED shown in FIG. 7.

Referring to FIG. 7, the emission layer EML may be disposed in an opening OH defined in a pixel defining layer PDL. For example, the emission layer EML which is divided by the pixel defining layer PDL and provided corresponding to each light emitting regions PXA-R, PXA-G, and PXA-B may emit light in a same wavelength range. In the display apparatus DD of an embodiment, the emission layer EML may emit blue light. While not shown in the drawings, in an embodiment, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, or the like. The light conversion body may emit provided light by converting the wavelength thereof. For example, the light control layer CCL may be a layer containing the quantum dot or a layer containing the phosphor.

The light control layer CCL may include light control units CCP1, CCP2, and CCP3. The light control units CCP1, CCP2, and CCP3 may be spaced apart from one another.

Referring to FIG. 7, divided patterns BMP may be disposed between the light control units CCP1, CCP2, and CCP3 which are spaced apart from each other, but embodiments are not limited thereto. FIG. 7 illustrates that the divided patterns BMP do not overlap the light control units CCP1, CCP2, and CCP3, but in an embodiment, at least a portion of the edges of the light control units CCP1, CCP2, and CCP3 may overlap the divided patterns BMP.

The light control layer CCL may include a first light control unit CCP1 containing a first quantum dot QD1 which converts first color light provided from the light emitting device ED into second color light, a second light control unit CCP2 containing a second quantum dot QD2 which converts the first color light into third color light, and a third light control unit CCP3 which transmits the first color light.

In an embodiment, the first light control unit CCP1 may provide red light that is the second color light, and the second light control unit CCP2 may provide green light that is the third color light. The third light control unit CCP3 may transmit blue light that is the first color light provided from the light-emitting element ED. For example, the first quantum dot QD1 may be a red quantum dot, and the second quantum dot QD2 may be a green quantum dot. The disclosure for the quantum dots described above may be applied with respect to the quantum dots QD1 and QD2.

The light control layer CCL may further include a scatterer SP. The first light control unit CCP1 may include the first quantum dot QD1 and the scatterer SP, the second light control unit CCP2 may include the second quantum dot QD2 and the scatterer SP, and the third light control unit CCP3 may not include any quantum dot but include the scatterer SP.

The scatterer SP may be inorganic particles. For example, the scatterer SP may include at least one of TiO₂, ZnO, Al₂O₃, SiO₂, and hollow silica. The scatterer SP may include any one of TiO₂, ZnO, Al₂O₃, SiO₂, and hollow silica, or may be a mixture of at least two materials selected from among TiO₂, ZnO, Al₂O₃, SiO₂, and hollow silica.

The first light control unit CCP1, the second light control unit CCP2, and the third light control unit CCP3 may respectively include base resins BR1, BR2, and BR3 in which the quantum dots QD1 and QD2 and the scatterer SP are dispersed. In an embodiment, the first light control unit CCP1 may include the first quantum dot QD1 and the scatterer SP dispersed in a first base resin BR1, the second light control unit CCP2 may include the second quantum dot QD2 and the scatterer SP dispersed in a second base resin BR2, and the third light control unit CCP3 may include the scatterer SP dispersed in a third base resin BR3. The base resins BR1, BR2, and BR3 are media in which the quantum dots QD1 and QD2 and the scatterer SP are dispersed, and may be formed of various resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 each may be the same as or different from each other.

The light control layer CCL may include a barrier layer BFL1. The barrier layer BFL1 may serve to prevent the penetration of moisture and/or oxygen (hereinafter, referred to as ‘moisture/oxygen’). The barrier layer BFL1 may be disposed on the light control units CCP1, CCP2, and CCP3 to block the light control units CCP1, CCP2 and CCP3 from being exposed to moisture/oxygen. The barrier layer BFL1 may cover the light control units CCP1, CCP2, and CCP3. The barrier layer BFL1 may be provided between the light control units CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. The barrier layers BFL1 and BFL2 may further include an organic film. The barrier layers BFL1 and BFL2 may be formed of a single layer or multiple layers.

In the display apparatus DD of an embodiment, the color filter layer CFL may be disposed on the light control layer CCL. For example, the color filter layer CFL may be directly disposed on the light control layer CCL. For example, the barrier layer BFL2 may be omitted.

The color filter layer CFL may include a light shielding unit BM and filters CF1, CF2, and CF3. The color filter layer CFL may include a first filter CF1 that transmits the second color light, a second filter CF2 that transmits the third color light, and a third filter CF3 that transmits the first color light. For example, the first filter CF1 may be a red filter, the second filter CF2 may be a green filter, and the third filter CF3 may be a blue filter. The filters CF1, CF2, and CF3 may each include a polymeric photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

In an embodiment, the first filter CF1 and the second filter CF2 may be a yellow filter. In another embodiment, the first filter CF1 and the second filter CF2 may not be separated but be provided as one filter.

The light shielding unit BM may be a black matrix. The light shielding unit BM may include an organic light shielding material or an inorganic light shielding material containing a black pigment or dye. The light shielding unit BM may prevent light leakage, and may separate boundaries between the adjacent filters CF1, CF2, and CF3. In an embodiment, the light shielding unit BM may be formed of a blue filter.

The first to third filters CF1, CF2, and CF3 may be disposed corresponding to the red light emitting region PXA-R, the green light emitting region PXA-G, and the blue light emitting region PXA-B, respectively.

A base substrate BL may be disposed on the color filter layer CFL. The base substrate BL may be a member which provides a base surface in which the color filter layer CFL, the light control layer CCL, and the like are disposed. The base substrate BL may be a glass substrate, a metal substrate, a plastic substrate, etc. However, embodiments are not limited thereto, and the base substrate BL may be an inorganic layer, an organic layer, or a composite material layer. While not shown in the drawings, in an embodiment, the base substrate BL may be omitted.

FIG. 8 is a schematic cross-sectional view illustrating a part of a display apparatus according to an embodiment. FIG. 8 illustrates a schematic cross-sectional view of a part corresponding to the display panel DP of FIG. 7. In the display apparatus DD-TD of an embodiment, the light emitting device ED-BT may include light emitting structures OL-B1, OL-B2, and OL-B3. The light emitting device ED-BT may include a first electrode EL1 and a second electrode EL2 which face each other, and the light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) therebetween.

For example, the light emitting device ED-BT included in the display apparatus DD-TD of an embodiment may be a light emitting device having a tandem structure and including multiple emission layers.

In an embodiment illustrated in FIG. 8, light emitted from each of the light emitting structures OL-B1, OL-B2, and OL-B3 may be a blue light. However, embodiments are not limited thereto, and the light emitted from each of the light emitting structures OL-B1, OL-B2, and OL-B3 may be in a wavelength range different from each other. For example, the light emitting device ED-BT including the light emitting structures OL-B1, OL-B2, and OL-B3 which emit light in a wavelength range different from each other may emit white light.

A charge generation layer CGL1 and CGL2 may be disposed between the neighboring light emitting structures OL-B1, OL-B2, and OL-B3. The charge generation layer CGL1 and CGL2 may include a p-type charge generation layer and/or an n-type charge generation layer.

At least one of the light emitting structures OL-B1, OL-B2, and OL-B3 included in the display apparatus DD-TD of an embodiment may contain the above-described polycyclic compound of an embodiment.

The light emitting device ED according to an embodiment may include the above-described polycyclic compound of an embodiment in at least one functional layer disposed between the first electrode EL1 and the second electrode EL2, thereby exhibiting improved luminous efficiency and service life characteristics. The light emitting device ED according to an embodiment may include the above-described polycyclic compound of an embodiment in at least one of the hole transport region HTR disposed between the first electrode EL1 and the second electrode EL2, the emission layer EML, and the electron transport region ETR, or in a capping layer CPL.

For example, the polycyclic compound according to an embodiment may be included in the hole transport region HTR of the light emitting device ED of an embodiment, and the light emitting device of an embodiment may exhibit excellent luminous efficiency and long service life characteristics.

The above-described polycyclic compound of an embodiment may have a molecular structure, in which the benzobisdibenzoheterol group derivative and the amine derivative are bonded, to thus improve film characteristics during forming a functional layer due to the three-dimensional molecular structure having a large asymmetry, thereby exhibiting improved luminous efficiency characteristics. Since the skeletal structure of the benzobisdibenzoheterol derivative and the bonding position of the benzobisdibenzoheterol derivative and the amine derivative are specified, the polycyclic compound of an embodiment may have improved stability of materials and hole transport ability between molecules, thereby contributing to long service life and high efficiency characteristics of the light emitting device.

Hereinafter, with reference to Examples and Comparative Examples, a polycyclic compound according to an embodiment and a light emitting device of an embodiment will be described in detail. The Examples shown below are illustrated only for understanding the disclosure, and the embodiments are not limited thereto.

### Examples

1. Synthesis of Polycyclic Compound

First, a synthesis method of the polycyclic compound according to the embodiment will be described in detail by illustrating the synthesis methods of Compounds A6, A26, A69, A111, and A141 of Compound Group 1A, Compounds B12, B45, B82, B114, and B149 of Compound Group 1B, Compounds C27, C49, C113, C138, and C159 of Compound Group 1C, Compounds D44, D85, D108, D137, and D146 of Compound Group 1D, Compound E144 of Compound Group 1E, Compound F150 of Compound Group 1F, Compound G19 of Compound Group 1G, and Compound H130 of Compound Group 1H. In the following descriptions, the synthesis methods of the polycyclic compounds are provided as examples, but the synthesis method according to an embodiment is not limited to Examples below.

<Synthesis of Compound A6>

Polycyclic Compound A6 according to an example may be synthesized by, for example, the steps shown in Reaction Scheme 1 below:

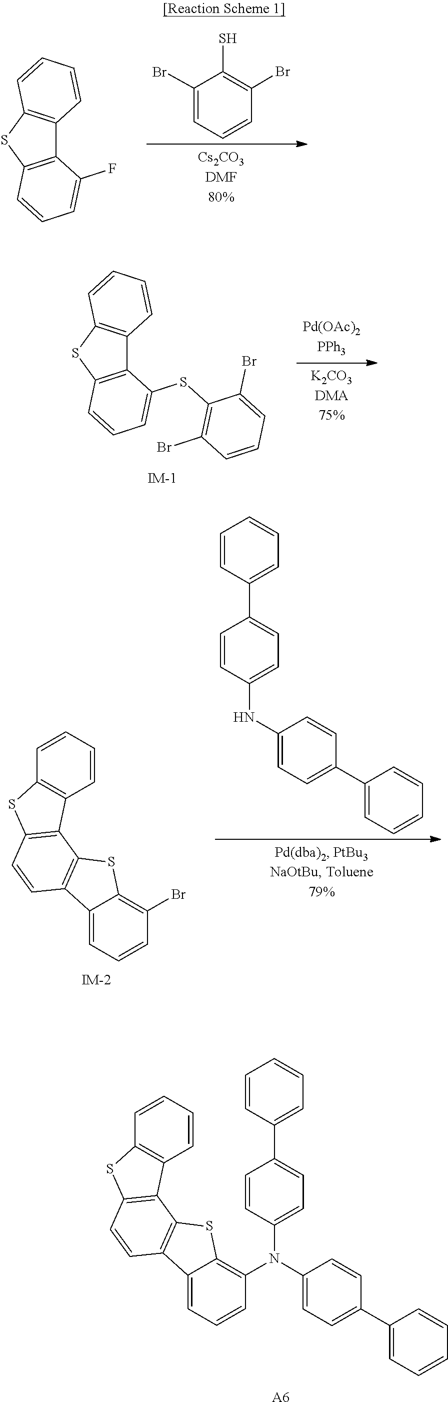

(Synthesis of Intermediate IM-1)

In an argon (Ar) atmosphere, in a 1000 mL three-neck flask, 2,6-dibromobenzenethiol (25.00 g, 93.3 mmol), 1-fluorodibenzothiophene (22.64 g, 1.2 equiv, 112.0 mmol), Cs₂CO₃ (60.79 g, 2.0 equiv, 186.6 mmol) and DMF (467 mL) were sequentially added, and heated and stirred at about 110° C. After air cooling to room temperature, water was added to the reaction solution, and the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-1 (33.60 g, yield 80%).

By measuring FAB-MS, a mass number of m/z=450 was observed by molecular ion peak, thereby identifying Intermediate IM-1.

(Synthesis of Intermediate IM-2)

In an Ar atmosphere, in a 500 mL three-neck flask, Intermediate IM-1 (25.00 g, 55.5 mmol), Pd(OAc)₂ (0.62 g, 0.05 equiv, 2.8 mmol), K₂CO₃ (11.51 g, 1.5 equiv, 83.3 mmol), PPh₃ (1.46 g, 0.10 equiv, 5.6 mmol), and DMA (222 mL) were sequentially added, and heated and stirred at about 140° C. After air cooling to room temperature, water was added to the reaction solvent, and the reaction solvent was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-2 (15.38 g, yield 75%).

By measuring FAB-MS, a mass number of m/z=369 was observed by molecular ion peak, thereby identifying Intermediate IM-2.

(Synthesis of Compound A6)

In an Ar atmosphere, in a 300 mL three-neck flask, IM-2 (10.00 g, 27.1 mmol), Pd(dba)₂ (0.47 g, 0.03 equiv, 0.8 mmol), NaOtBu (5.20 g, 2.0 equiv, 54.2 mmol), toluene (135 mL), bis(4-biphenylyl)amine (9.57 g, 1.1 equiv, 29.8 mmol), and tBu₃P (0.55 g, 0.1 equiv, 2.7 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound A6 (13.04 g, yield 79%).

By measuring FAB-MS, a mass number of m/z=609 was observed by molecular ion peak, thereby identifying Compound A6.

<Synthesis of Compound A26>

Polycyclic Compound A26 according to an example may be synthesized by, for example, the steps shown in Reaction Scheme 2 below:

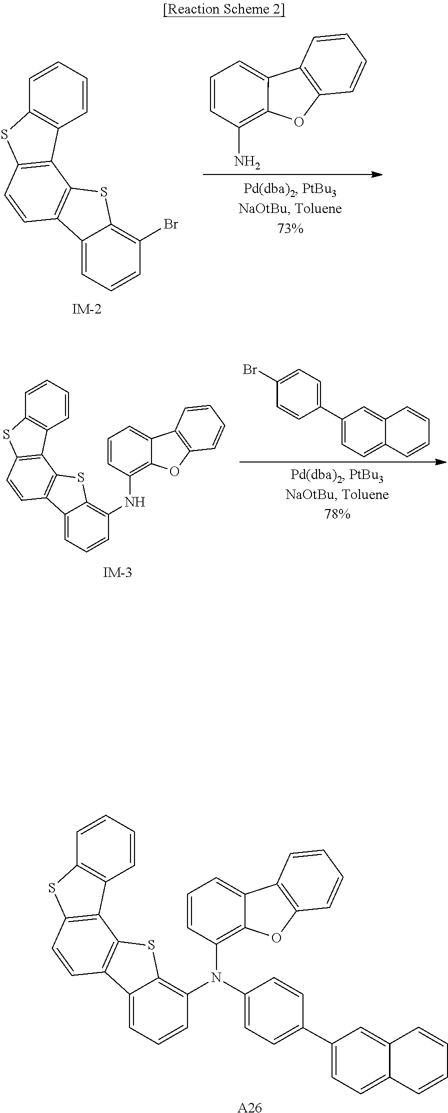

(Synthesis of Intermediate IM-3)

In an Ar atmosphere, in a 500 mL three-neck flask, Intermediate IM-2 (13.00 g, 35.2 mmol), Pd(dba)₂ (0.61 g, 0.03 equiv, 1.1 mmol), NaOtBu (3.38 g, 1.0 equiv, 35.2 mmol), toluene (176 mL), dibenzofuran-4-amine (7.09 g, 1.1 equiv, 38.7 mmol), and tBu₃P (0.71 g, 0.1 equiv, 3.5 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-3 (12.12 g, yield 73%).

By measuring FAB-MS, a mass number of m/z=471 was observed by molecular ion peak, thereby identifying Intermediate IM-3.

(Synthesis of Compound A26)

In an Ar atmosphere, in a 300 mL three-neck flask, Intermediate IM-2 (10.00 g, 21.2 mmol), Pd(dba)₂ (0.37 g, 0.03 equiv, 0.6 mmol), NaOtBu (4.08 g, 2.0 equiv, 42.4 mmol), toluene (106 mL), 2-(4-bromophenyl)naphthalene (6.61 g, 1.1 equiv, 23.3 mmol), and tBu₃P (0.43 g, 0.1 equiv, 2.1 mmol) were sequentially added, and heated and stirred under reflux. After air cooling to room temperature, organic layers were separated and obtained by adding water to the reaction solvent. The organic layers were further extracted by further toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound A26 (11.15 g, yield 78%).

By measuring FAB-MS, a mass number of m/z=673 was observed by molecular ion peak, thereby identifying Compound A26.

<Synthesis of Compound A69>

Polycyclic Compound A69 according to an example may be synthesized by, for example, the steps shown in Reaction Scheme 3 below:

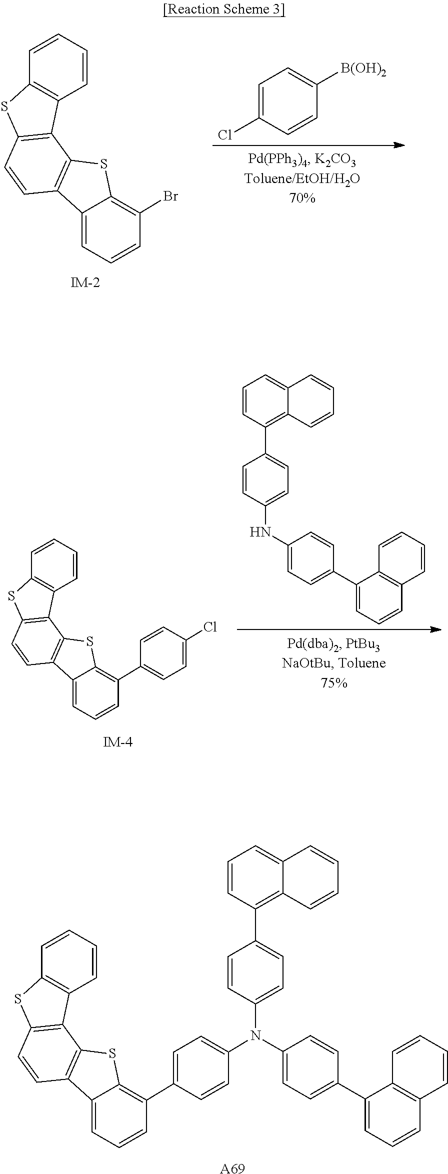

(Synthesis of Intermediate IM-4)

In an Ar atmosphere, in a 500 mL three-neck flask, Intermediate IM-2 (15.00 g, 40.6 mmol), 4-chlorophenylboronic acid (6.99 g, 1.1 equiv, 44.7 mmol), K₂CO₃ (16.84 g, 3.0 equiv, 121.9 mmol), Pd(PPh₃)₄ (2.35 g, 0.05 equiv, 2.0 mmol), and a mixed solution of toluene/ethanol/water (4/2/1, 284 mL) were sequentially added, and heated and stirred at about 80° C. After air cooling to room temperature, the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-4 (11.40 g, yield 70%).

By measuring FAB-MS, a mass number of m/z=400 was observed by molecular ion peak, thereby identifying Intermediate IM-4.

(Synthesis of Compound A69)

In an Ar atmosphere, in a 300 mL three-neck flask, Intermediate IM-4 (10.00 g, 24.9 mmol), Pd(dba)₂ (0.43 g, 0.03 equiv, 0.7 mmol), NaOtBu (4.79 g, 2.0 equiv, 49.9 mmol), toluene (125 mL), bis[4-(naphthalen-1-yl)phenyl]amine (11.56 g, 1.1 equiv, 27.4 mmol), and tBu₃P (0.51 g, 0.1 equiv, 2.5 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound A69 (14.70 g, yield 75%).

By measuring FAB-MS, a mass number of m/z=786 was observed by molecular ion peak, thereby identifying Compound A69.

<Synthesis of Compound A111>

Polycyclic Compound A111 according to an example may be synthesized by, for example, the steps shown in Reaction Scheme 4 below:

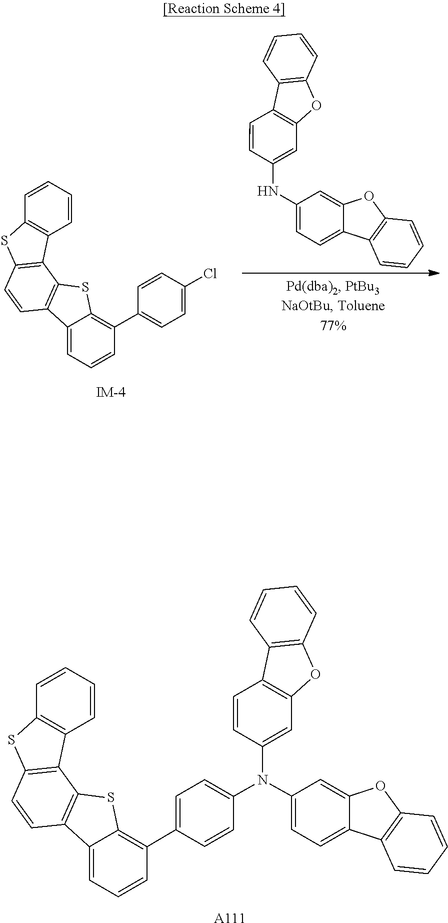

In an Ar atmosphere, in a 300 mL three-neck flask, Intermediate IM-4 (10.00 g, 24.9 mmol), Pd(dba)₂ (0.43 g, 0.03 equiv, 0.7 mmol), NaOtBu (4.79 g, 2.0 equiv, 49.9 mmol), toluene (125 mL), bis(dibenzofuran-3-yl)amine (9.59 g, 1.1 equiv, 27.4 mmol), and tBu₃P (0.51 g, 0.1 equiv, 2.5 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound A111 (13.71 g, yield 77%).

By measuring FAB-MS, a mass number of m/z=713 was observed by molecular ion peak, thereby identifying Compound A111.

<Synthesis of Compound A141>

Polycyclic Compound A141 according to an example may be synthesized by, for example, the steps shown in Reaction Scheme 5 below:

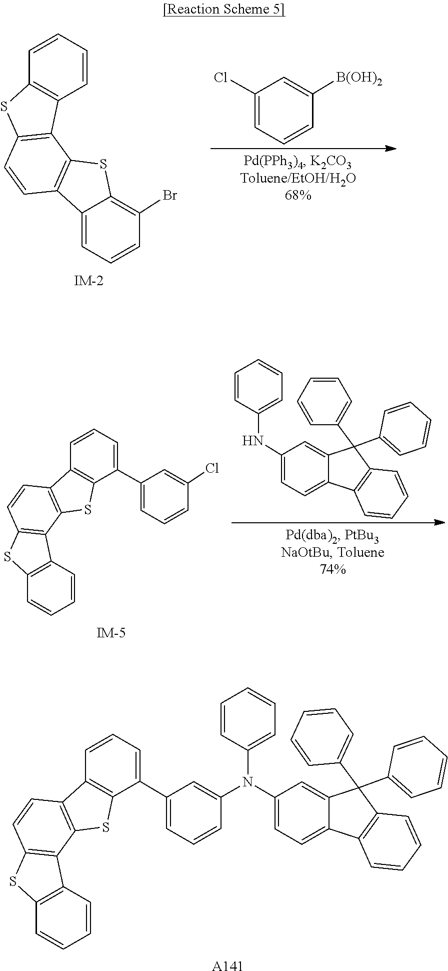

(Synthesis of Intermediate IM-5)

In an Ar atmosphere, in a 500 mL three-neck flask, Intermediate IM-2 (15.00 g, 40.6 mmol), 3-chlorophenylboronic acid (6.99 g, 1.1 equiv, 44.7 mmol), K₂CO₃ (16.84 g, 3.0 equiv, 121.9 mmol), Pd(PPh₃)₄ (2.35 g, 0.05 equiv, 2.0 mmol), and a mixed solution of toluene/ethanol/water (4/2/1, 284 mL) were sequentially added, and heated and stirred at about 80° C. After air cooling to room temperature, the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-5 (11.07 g, yield 68%).

By measuring FAB-MS, a mass number of m/z=400 was observed by molecular ion peak, thereby identifying Intermediate IM-5.

(Synthesis of Compound A141)

In an Ar atmosphere, in a 300 mL three-neck flask, Intermediate IM-5 (10.00 g, 24.9 mmol), Pd(dba)₂ (0.43 g, 0.03 equiv, 0.7 mmol), NaOtBu (4.79 g, 2.0 equiv, 49.9 mmol), toluene (125 mL), N,9,9-triphenyl-9H-fluoren-2-amine (11.24 g, 1.1 equiv, 27.4 mmol), and tBu₃P (0.51 g, 0.1 equiv, 2.5 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound A141 (14.29 g, yield 74%).

By measuring FAB-MS, a mass number of m/z=774 was observed by molecular ion peak, thereby identifying Compound A141.

<Synthesis of Compound B12>

Polycyclic Compound B12 according to an example may be synthesized by, for example, the steps shown in Reaction Scheme 6 below:

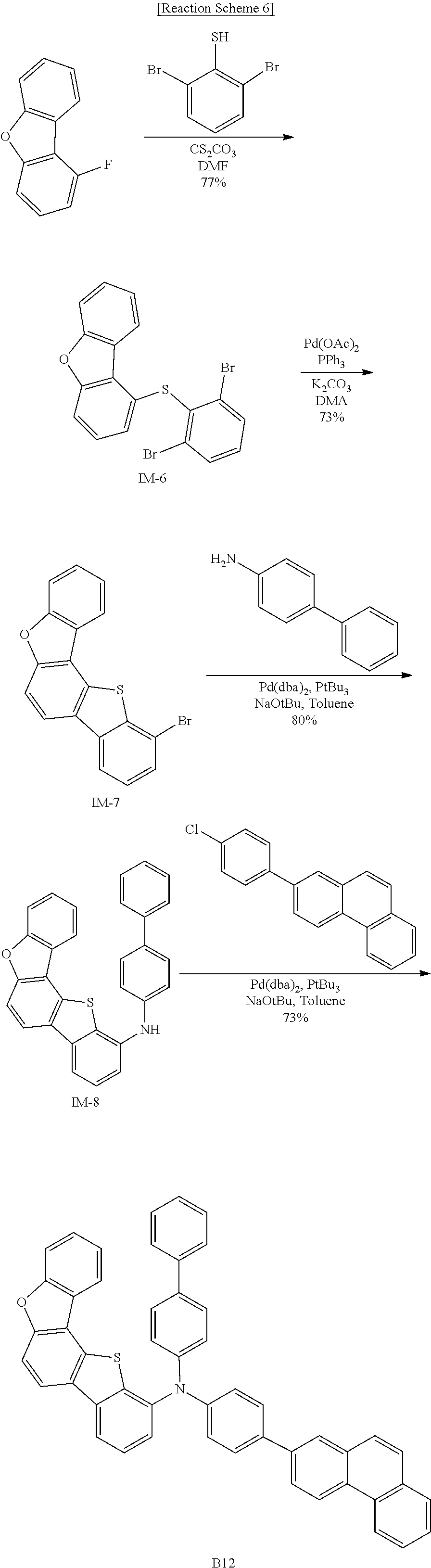

(Synthesis of Intermediate IM-6)

In an Ar atmosphere, in a 1000 mL three-neck flask, 2,6-dibromobenzenethiol (25.00 g, 93.3 mmol), 1-fluorodibenzofuran (20.84 g, 1.2 equiv, 112.0 mmol), Cs₂CO₃ (90.79 g, 2.0 equiv, 186.6 mmol) and DMF (466 mL) were sequentially added, and heated and stirred at about 110° C. After air cooling to room temperature, water was added to the reaction solution, and the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-6 (32.40 g, yield 77%).

By measuring FAB-MS, a mass number of m/z=434 was observed by molecular ion peak, thereby identifying Intermediate IM-6.

(Synthesis of Intermediate IM-7)

In an Ar atmosphere, in a 500 mL three-neck flask, Intermediate IM-6 (25.00 g, 57.6 mmol), Pd(OAc)₂ (0.65 g, 0.05 equiv, 2.9 mmol), K₂CO₃ (11.94 g, 1.5 equiv, 86.4 mmol), PPh₃ (1.51 g, 0.10 equiv, 5.8 mmol), and DMA (230 mL) were sequentially added, and heated and stirred at about 140° C. After air cooling to room temperature, water was added to the reaction solvent, and the reaction solvent was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-7 (14.85 g, yield 73%).

By measuring FAB-MS, a mass number of m/z=353 was observed by molecular ion peak, thereby identifying Intermediate IM-7.

(Synthesis of Intermediate IM-8)

In an Ar atmosphere, in a 500 mL three-neck flask, Intermediate IM-7 (13.00 g, 36.8 mmol), Pd(dba)₂ (0.63 g, 0.03 equiv, 1.1 mmol), NaOtBu (3.54 g, 1.0 equiv, 36.8 mmol), toluene (184 mL), 4-biphenylamine (6.85 g, 1.1 equiv, 40.5 mmol), and tBu₃P (0.74 g, 0.1 equiv, 3.7 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-8 (13.00 g, yield 80%).

By measuring FAB-MS, a mass number of m/z=441 was observed by molecular ion peak, thereby identifying Intermediate IM-8.

(Synthesis of Compound B12)

In an Ar atmosphere, in a 300 mL three-neck flask, Intermediate IM-8 (10.00 g, 22.6 mmol), Pd(dba)₂ (0.39 g, 0.03 equiv, 0.7 mmol), NaOtBu (4.35 g, 2.0 equiv, 45.3 mmol), toluene (113 mL), 2-(4-chlorophenyl)phenanthrene (7.19 g, 1.1 equiv, 24.9 mmol), and tBu₃P (0.46 g, 0.1 equiv, 2.3 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound B12 (11.47 g, yield 73%).

By measuring FAB-MS, a mass number of m/z=693 was observed by molecular ion peak, thereby identifying Compound B12.

<Synthesis of Compound B45>

Polycyclic Compound B45 according to an example may be synthesized by, for example, the steps shown in Reaction Scheme 7 below:

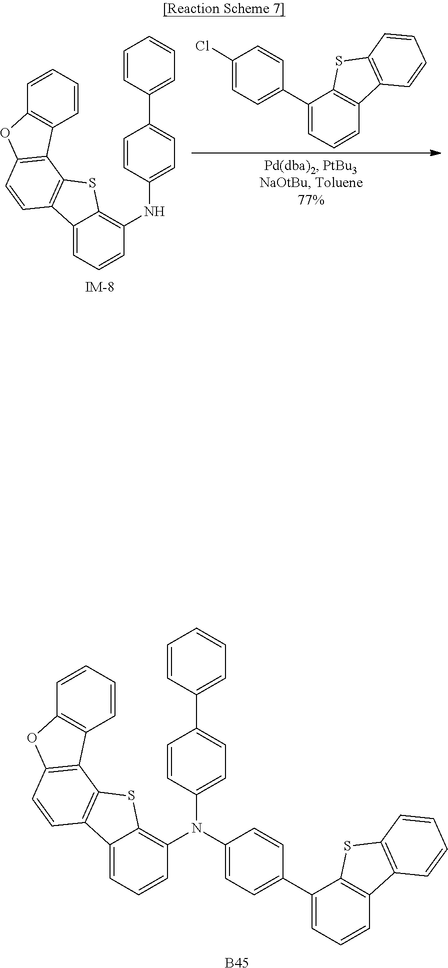

In an Ar atmosphere, in a 300 mL three-neck flask, Intermediate IM-8 (10.00 g, 22.6 mmol), Pd(dba)₂ (0.39 g, 0.03 equiv, 0.7 mmol), NaOtBu (4.35 g, 2.0 equiv, 45.3 mmol), toluene (113 mL), (4-chlorophenyl)dibenzothiophene (7.34 g, 1.1 equiv, 24.9 mmol), and tBu₃P (0.46 g, 0.1 equiv, 2.3 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound B45 (12.21 g, yield 77%).

By measuring FAB-MS, a mass number of m/z=699 was observed by molecular ion peak, thereby identifying Compound B45.

<Synthesis of Compound B82>

Polycyclic Compound B82 according to an example may be synthesized by, for example, the steps shown in Reaction Scheme 8 below:

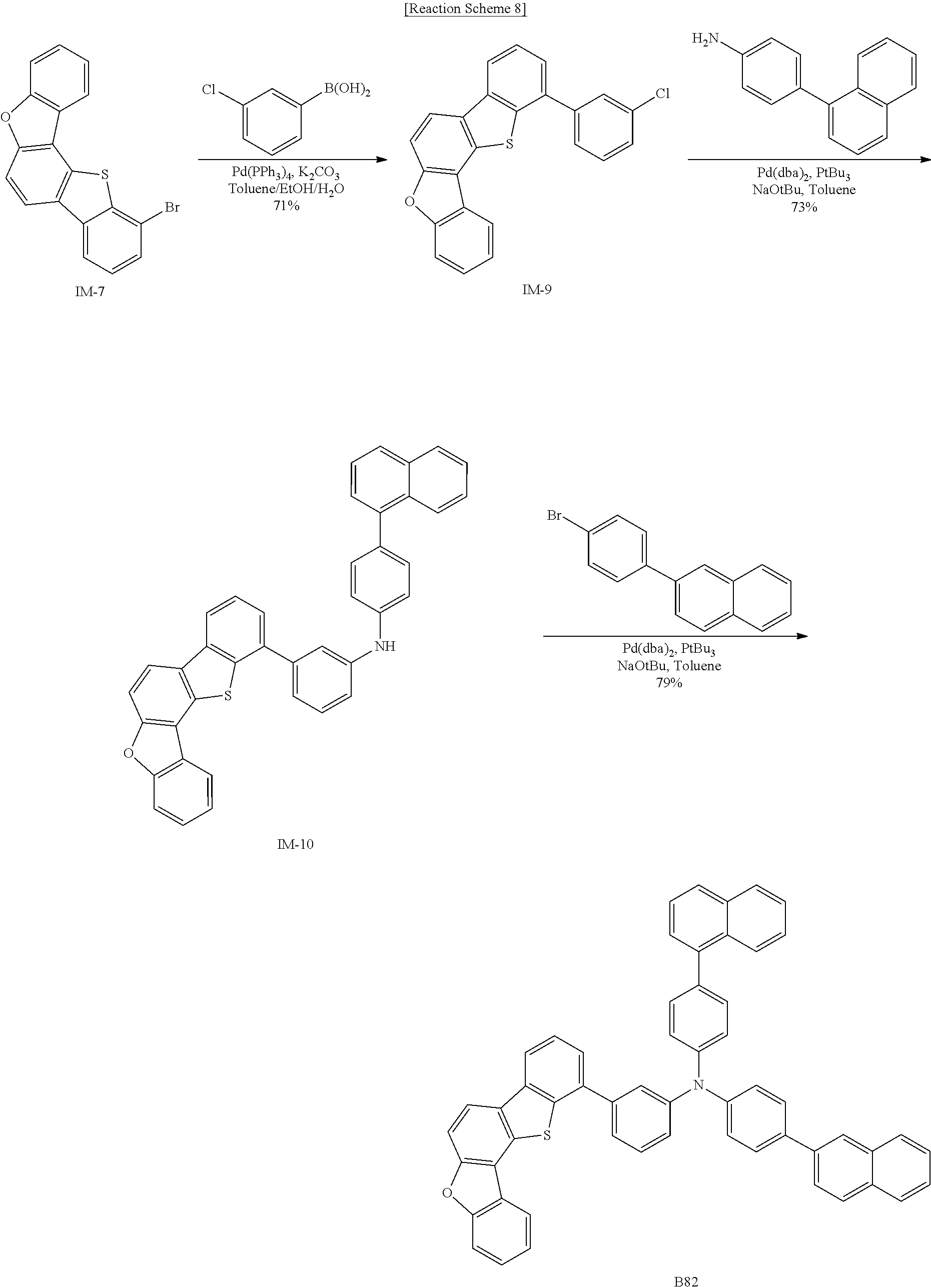

(Synthesis of Intermediate IM-9)

In an Ar atmosphere, in a 500 mL three-neck flask, Intermediate IM-7 (15.00 g, 42.5 mmol), 3-chlorophenylboronic acid (7.30 g, 1.1 equiv, 46.7 mmol), K₂CO₃ (17.61 g, 3.0 equiv, 127.4 mmol), Pd(PPh₃)₄ (2.45 g, 0.05 equiv, 2.1 mmol), and a mixed solution of toluene/ethanol/water (4/2/1, 298 mL) were sequentially added, and heated and stirred at about 80° C. After air cooling to room temperature, the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-9 (11.60 g, yield 71%).

By measuring FAB-MS, a mass number of m/z=384 was observed by molecular ion peak, thereby identifying Intermediate IM-9.

(Synthesis of Intermediate IM-10)

In an Ar atmosphere, in a 500 mL three-neck flask, Intermediate IM-9 (13.00 g, 33.8 mmol), Pd(dba)₂ (0.58 g, 0.03 equiv, 1.0 mmol), NaOtBu (3.25 g, 1.0 equiv, 33.8 mmol), toluene (169 mL), (naphthalen-1-yl)aniline (8.15 g, 1.1 equiv, 37.2 mmol), and tBu₃P (0.69 g, 0.1 equiv, 3.4 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-10 (14.00 g, yield 73%).

By measuring FAB-MS, a mass number of m/z=567 was observed by molecular ion peak, thereby identifying Intermediate IM-10.

(Synthesis of Compound B82)

In an Ar atmosphere, in a 300 mL three-neck flask, Intermediate IM-10 (10.00 g, 17.6 mmol), Pd(dba)₂ (0.30 g, 0.03 equiv, 0.5 mmol), NaOtBu (3.39 g, 2.0 equiv, 35.2 mmol), toluene (88 mL), 2-(4-bromophenyl)naphthalene (5.49 g, 1.1 equiv, 19.4 mmol), and tBu₃P (0.36 g, 0.1 equiv, 1.8 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound B82 (10.71 g, yield 79%).

By measuring FAB-MS, a mass number of m/z=769 was observed by molecular ion peak, thereby identifying Compound B82.

<Synthesis of Compound B114>

Polycyclic Compound B114 according to an example may be synthesized by, for example, the steps shown in Reaction Scheme 9 below:

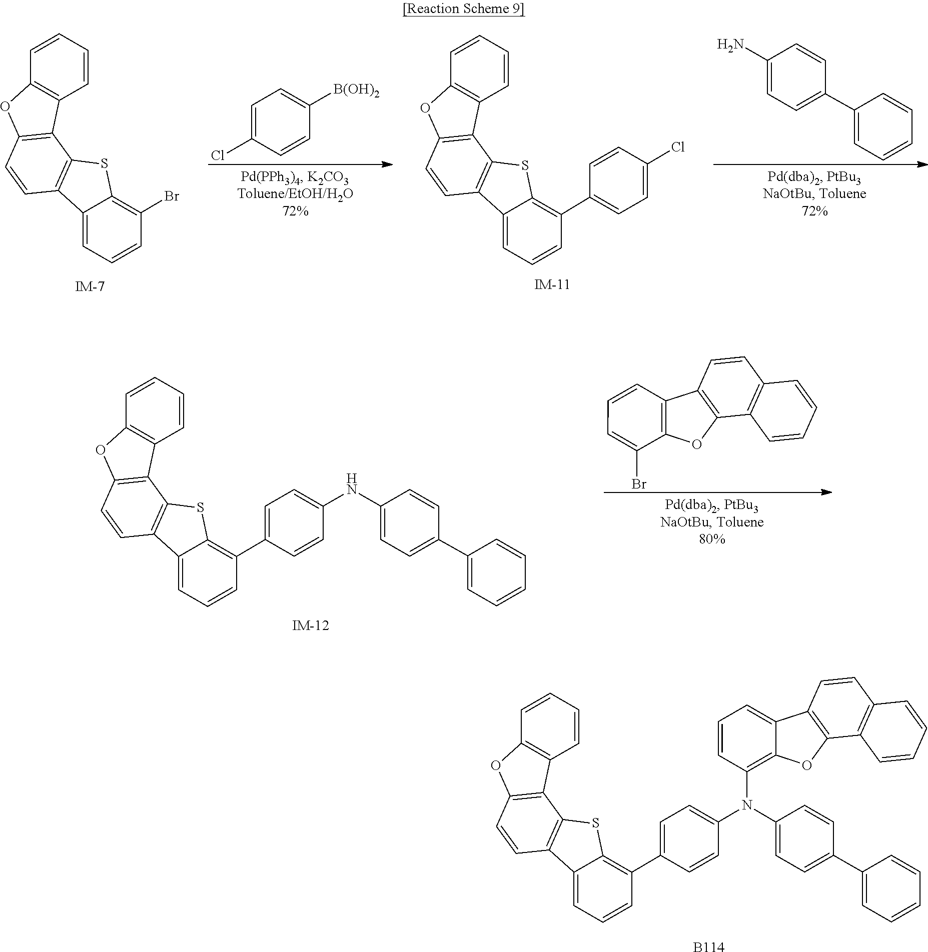

(Synthesis of Intermediate IM-11)

In an Ar atmosphere, in a 500 mL three-neck flask, Intermediate IM-7 (15.00 g, 42.5 mmol), 4-chlorophenylboronic acid (7.30 g, 1.1 equiv, 46.7 mmol), K₂CO₃ (17.61 g, 3.0 equiv, 127.4 mmol), Pd(PPh₃)₄ (2.45 g, 0.05 equiv, 2.1 mmol), and a mixed solution of toluene/ethanol/water (4/2/1, 298 mL) were sequentially added, and heated and stirred at about 80° C. After air cooling to room temperature, the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-11 (11.77 g, yield 72%).

By measuring FAB-MS, a mass number of m/z=384 was observed by molecular ion peak, thereby identifying Intermediate IM-11.

(Synthesis of Intermediate IM-12)

In an Ar atmosphere, in a 500 mL three-neck flask, Intermediate IM-11 (13.00 g, 33.8 mmol), Pd(dba)₂ (0.58 g, 0.03 equiv, 1.0 mmol), NaOtBu (3.25 g, 1.0 equiv, 33.8 mmol), toluene (169 mL), 4-biphenylamine (6.29 g, 1.1 equiv, 37.2 mmol), and tBu₃P (0.69 g, 0.1 equiv, 3.4 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-12 (12.59 g, yield 72%).

By measuring FAB-MS, a mass number of m/z=517 was observed by molecular ion peak, thereby identifying Intermediate IM-12.

(Synthesis of Compound B114)

In an Ar atmosphere, in a 300 mL three-neck flask, Intermediate IM-12 (10.00 g, 19.3 mmol), Pd(dba)₂ (0.33 g, 0.03 equiv, 0.6 mmol), NaOtBu (3.71 g, 2.0 equiv, 38.6 mmol), toluene (97 mL), 10-bromonaphtho[1,2-b]benzofuran (6.31 g, 1.1 equiv, 21.2 mmol), and tBu₃P (0.39 g, 0.1 equiv, 1.9 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound B114 (11.34 g, yield 80%).

By measuring FAB-MS, a mass number of m/z=733 was observed by molecular ion peak, thereby identifying Compound B114.

<Synthesis of Compound B149>

Polycyclic Compound B149 according to an example may be synthesized by, for example, the steps shown in Reaction Scheme 10 below:

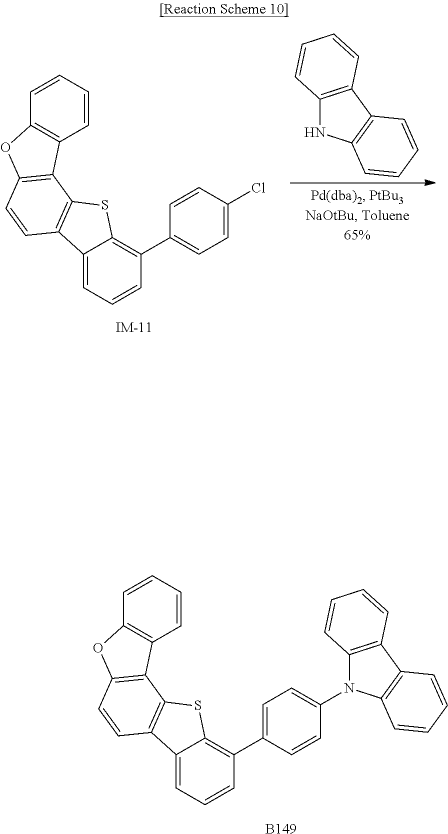

In an Ar atmosphere, in a 300 mL three-neck flask, Intermediate IM-11 (13.00 g, 36.8 mmol), Pd(dba)₂ (0.58 g, 0.03 equiv, 1.1 mmol), NaOtBu (6.49 g, 2.0 equiv, 36.8 mmol), toluene (169 mL), 9H-carbazole (6.21 g, 1.1 equiv, 37.2 mmol), and tBu₃P (0.68 g, 0.1 equiv, 3.4 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound B149 (11.32 g, yield 65%).

By measuring FAB-MS, a mass number of m/z=515 was observed by molecular ion peak, thereby identifying Compound B149.

<Synthesis of Compound C27>

Polycyclic Compound C27 according to an example may be synthesized by, for example, the steps shown in Reaction Scheme 11 below:

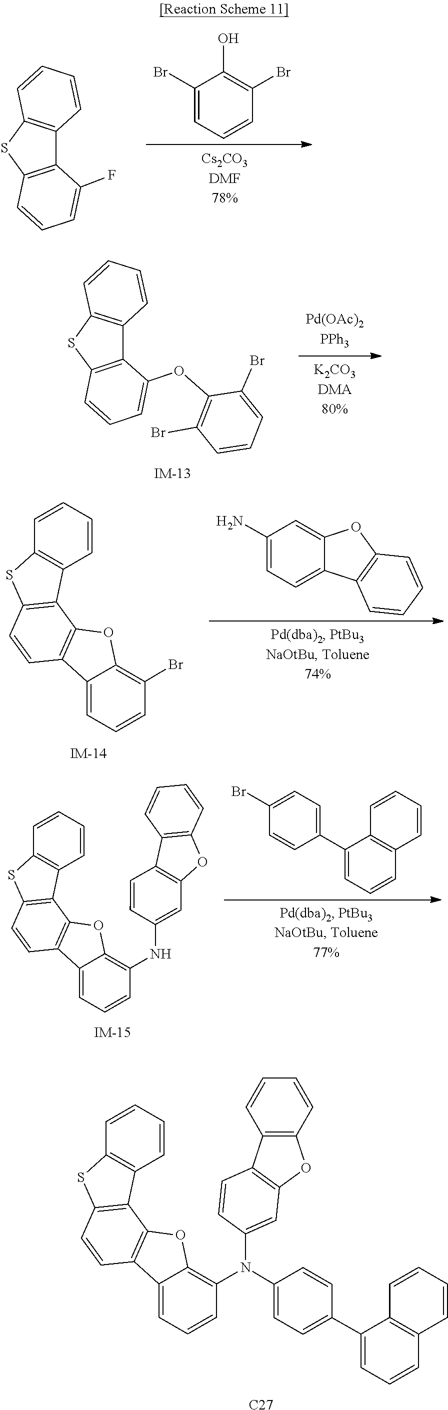

(Synthesis of Intermediate IM-13)

In an Ar atmosphere, in a 1000 mL three-neck flask, 2,6-dibromophenol (25.00 g, 99.24 mmol), 1-fluorodibenzothiophene (24.09 g, 1.2 equiv, 119.1 mmol), Cs₂CO₃ (64.67 g, 2.0 equiv, 198.5 mmol) and DMF (496 mL) were sequentially added, and heated and stirred at about 110° C. After air cooling to room temperature, water was added to the reaction solution, and the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-13 (33.61 g, yield 78%).

By measuring FAB-MS, a mass number of m/z=434 was observed by molecular ion peak, thereby identifying Intermediate IM-13.

(Synthesis of Intermediate IM-14)

In an Ar atmosphere, in a 500 mL three-neck flask, Intermediate IM-13 (25.00 g, 57.6 mmol), Pd(OAc)₂ (0.65 g, 0.05 equiv, 2.9 mmol), K₂CO₃ (11.94 g, 1.5 equiv, 86.4 mmol), PPh₃ (1.51 g, 0.10 equiv, 5.8 mmol), and DMA (230 mL) were sequentially added, and heated and stirred at about 140° C. After air cooling to room temperature, water was added to the reaction solvent, and the reaction solvent was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-14 (16.27 g, yield 80%).

By measuring FAB-MS, a mass number of m/z=353 was observed by molecular ion peak, thereby identifying Intermediate IM-14.

(Synthesis of Intermediate IM-15)

In an Ar atmosphere, in a 500 mL three-neck flask, Intermediate IM-14 (13.00 g, 36.8 mmol), Pd(dba)₂ (0.63 g, 0.03 equiv, 1.1 mmol), NaOtBu (3.54 g, 1.0 equiv, 36.8 mmol), toluene (184 mL), dibenzofuran-3-amine (7.42 g, 1.1 equiv, 40.5 mmol), and tBu₃P (0.74 g, 0.1 equiv, 3.7 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-15 (12.41 g, yield 74%).

By measuring FAB-MS, a mass number of m/z=455 was observed by molecular ion peak, thereby identifying Intermediate IM-15.

(Synthesis of Compound C27)

In an Ar atmosphere, in a 300 mL three-neck flask, Intermediate IM-15 (10.00 g, 22.0 mmol), Pd(dba)₂ (0.38 g, 0.03 equiv, 0.7 mmol), NaOtBu (4.22 g, 2.0 equiv, 43.9 mmol), toluene (110 mL), 1-(4-bromophenyl)naphthalene (6.84 g, 1.1 equiv, 24.1 mmol), and tBu₃P (0.44 g, 0.1 equiv, 2.2 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound C27 (11.12 g, yield 77%).

By measuring FAB-MS, a mass number of m/z=657 was observed by molecular ion peak, thereby identifying Compound C27.

<Synthesis of Compound C49>

Polycyclic Compound C49 according to an example may be synthesized by, for example, the steps shown in Reaction Scheme 12 below:

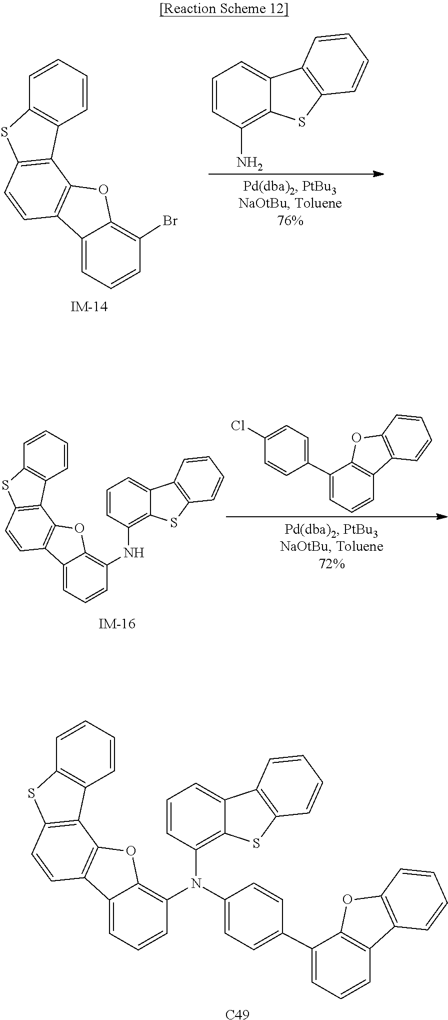

(Synthesis of Intermediate IM-16)

In an Ar atmosphere, in a 500 mL three-neck flask, Intermediate IM-14 (13.00 g, 36.8 mmol), Pd(dba)₂ (0.63 g, 0.03 equiv, 1.1 mmol), NaOtBu (3.54 g, 1.0 equiv, 36.8 mmol), toluene (184 mL), dibenzothiophen-4-amine (8.07 g, 1.1 equiv, 40.5 mmol), and tBu₃P (0.74 g, 0.1 equiv, 3.7 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-16 (13.19 g, yield 76%).

By measuring FAB-MS, a mass number of m/z=471 was observed by molecular ion peak, thereby identifying Intermediate IM-16.

(Synthesis of Compound C49)

In an Ar atmosphere, in a 300 mL three-neck flask, Intermediate IM-16 (10.00 g, 21.2 mmol), Pd(dba)₂ (0.37 g, 0.03 equiv, 0.6 mmol), NaOtBu (4.08 g, 2.0 equiv, 42.4 mmol), toluene (106 mL), (4-chlorophenyl)dibenzofuran (6.50 g, 1.1 equiv, 23.3 mmol), and tBu₃P (0.43 g, 0.1 equiv, 2.1 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound C49 (10.90 g, yield 72%).

By measuring FAB-MS, a mass number of m/z=713 was observed by molecular ion peak, thereby identifying Compound C49.

<Synthesis of Compound C113>

Polycyclic Compound C113 according to an example may be synthesized by, for example, the steps shown in Reaction Scheme 13 below:

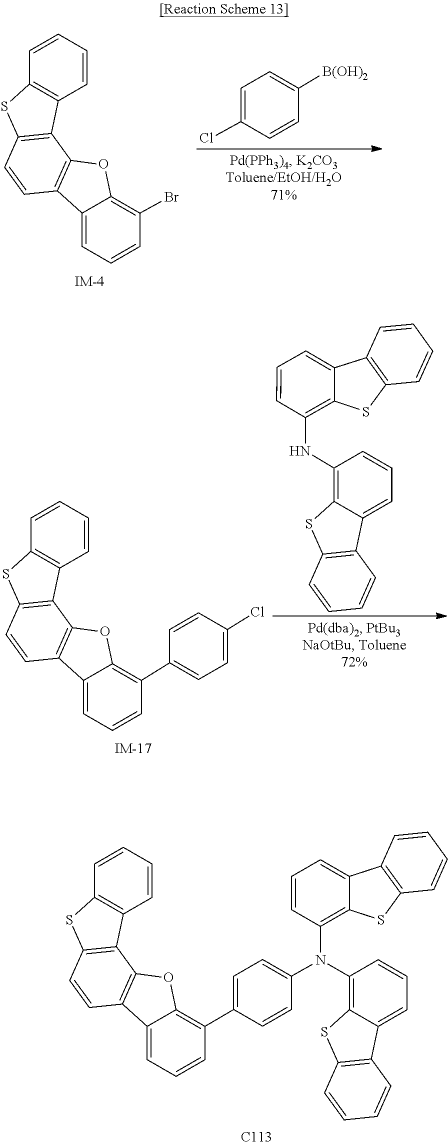

(Synthesis of Intermediate IM-17)

In an Ar atmosphere, in a 500 mL three-neck flask, Intermediate IM-14 (15.00 g, 42.5 mmol), 4-chlorophenylboronic acid (7.30 g, 1.1 equiv, 46.7 mmol), K₂CO₃ (17.61 g, 3.0 equiv, 127.4 mmol), Pd(PPh₃)₄ (2.45 g, 0.05 equiv, 2.1 mmol), and a mixed solution of toluene/ethanol/water (4/2/1, 298 mL) were sequentially added, and heated and stirred at about 80° C. After air cooling to room temperature, the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-17 (11.60 g, yield 71%).

By measuring FAB-MS, a mass number of m/z=384 was observed by molecular ion peak, thereby identifying Intermediate IM-17.

(Synthesis of Compound C113)

In an Ar atmosphere, in a 300 mL three-neck flask, Intermediate IM-17 (13.00 g, 33.8 mmol), Pd(dba)₂ (0.58 g, 0.03 equiv, 1.1 mmol), NaOtBu (6.49 g, 2.0 equiv, 67.6 mmol), toluene (169 mL), bis(dibenzofuran-4-yl)amine (14.17 g, 1.1 equiv, 37.2 mmol), and tBu₃P (0.68 g, 0.1 equiv, 3.4 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound C113 (17.75 g, yield 72%).

By measuring FAB-MS, a mass number of m/z=729 was observed by molecular ion peak, thereby identifying Compound C113.

<Synthesis of Compound C138>

Polycyclic Compound C138 according to an example may be synthesized by, for example, the steps shown in Reaction Scheme 14 below:

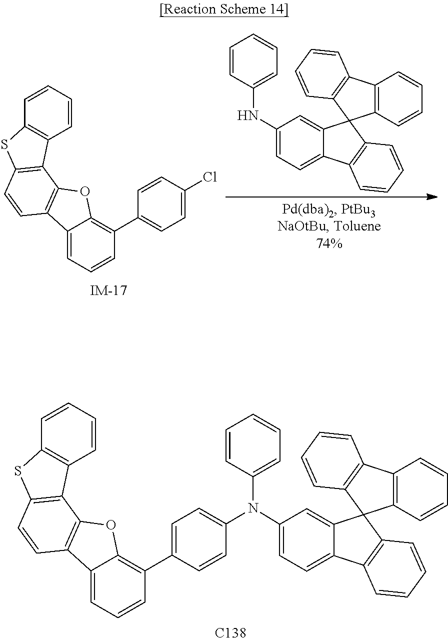

In an Ar atmosphere, in a 300 mL three-neck flask, Intermediate IM-17 (13.00 g, 33.8 mmol), Pd(dba)₂ (0.58 g, 0.03 equiv, 1.1 mmol), NaOtBu (6.49 g, 2.0 equiv, 67.6 mmol), toluene (169 mL), N-phenyl-9,9′-spirobi[fluoren]-2-amine (15.14 g, 1.1 equiv, 37.2 mmol), and tBu₃P (0.68 g, 0.1 equiv, 3.4 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound C138 (18.89 g, yield 74%).

By measuring FAB-MS, a mass number of m/z=755 was observed by molecular ion peak, thereby identifying Compound C138.

<Synthesis of Compound C159>

Polycyclic Compound C159 according to an example may be synthesized by, for example, the steps shown in Reaction Scheme 15 below:

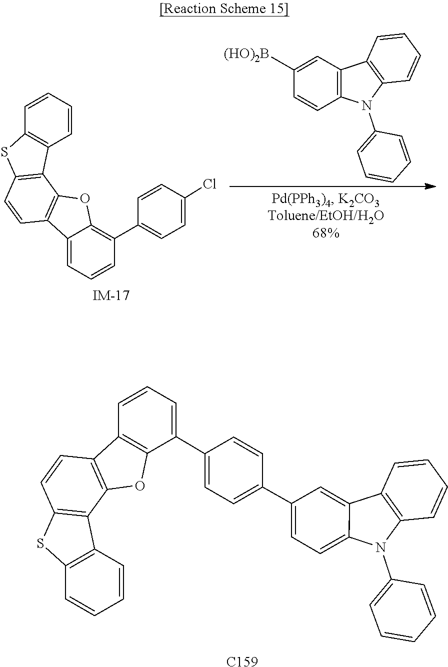

In an Ar atmosphere, in a 500 mL three-neck flask, Intermediate IM-17 (15.00 g, 39.0 mmol), (9-phenyl-9H-carbazol-3-yl)boronic acid (12.31 g, 1.1 equiv, 42.9 mmol), K₂CO₃ (16.16 g, 3.0 equiv, 116.9 mmol), Pd(PPh₃)₄ (2.25 g, 0.05 equiv, 1.9 mmol), and a mixed solution of toluene/ethanol/water (4/2/1, 272 mL) were sequentially added, and heated and stirred at about 80° C. After air cooling to room temperature, the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain solid Compound C159 (15.68 g, yield 68%).

By measuring FAB-MS, a mass number of m/z=591 was observed by molecular ion peak, thereby identifying Compound C159.

<Synthesis of Compound D44>

Polycyclic Compound D44 according to an example may be synthesized by, for example, the steps shown in Reaction Scheme 16 below:

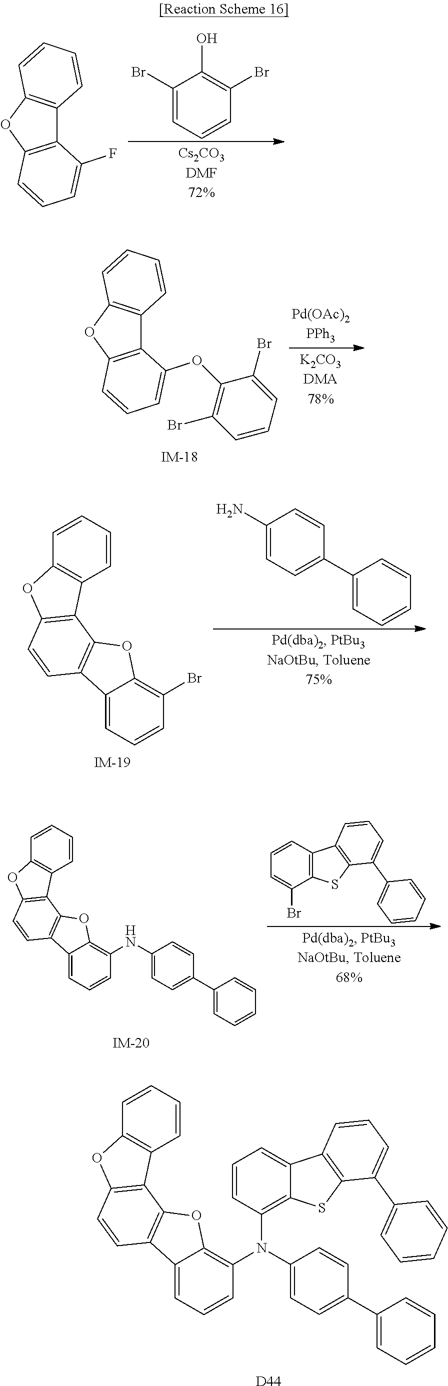

(Synthesis of Intermediate IM-18)

In an Ar atmosphere, in a 1000 mL three-neck flask, 2,6-dibromophenol (25.00 g, 99.2 mmol), 1-fluorodibenzofuran (22.17 g, 1.2 equiv, 119.1 mmol), Cs₂CO₃ (64.67 g, 2.0 equiv, 198.5 mmol) and DMF (496 mL) were sequentially added, and heated and stirred at about 110° C. After air cooling to room temperature, water was added to the reaction solution, and the reaction solution was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-18 (29.87 g, yield 72%).

By measuring FAB-MS, a mass number of m/z=418 was observed by molecular ion peak, thereby identifying Intermediate IM-18.

(Synthesis of Intermediate IM-19)

In an Ar atmosphere, in a 500 mL three-neck flask, Intermediate IM-18 (25.00 g, 59.8 mmol), Pd(OAc)₂ (0.67 g, 0.05 equiv, 3.0 mmol), K₂CO₃ (12.40 g, 1.5 equiv, 89.7 mmol), PPh₃ (1.57 g, 0.10 equiv, 6.0 mmol), and DMA (240 mL) were sequentially added, and heated and stirred at about 140° C. After air cooling to room temperature, water was added to the reaction solvent, and the reaction solvent was extracted with toluene to obtain organic layers. A water layer was removed, the organic layers were washed with saturated saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-19 (15.73 g, yield 78%).

By measuring FAB-MS, a mass number of m/z=337 was observed by molecular ion peak, thereby identifying Intermediate IM-19.

(Synthesis of Intermediate IM-20)

In an Ar atmosphere, in a 500 mL three-neck flask, Intermediate IM-19 (13.00 g, 38.6 mmol), Pd(dba)₂ (0.67 g, 0.03 equiv, 1.2 mmol), NaOtBu (3.71 g, 1.0 equiv, 38.6 mmol), toluene (193 mL), 4-biphenylamine (7.12 g, 1.1 equiv, 42.4 mmol), and tBu₃P (0.78 g, 0.1 equiv, 3.9 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-20 (12.30 g, yield 75%).

By measuring FAB-MS, a mass number of m/z=425 was observed by molecular ion peak, thereby identifying Intermediate IM-20.

(Synthesis of Compound D44)

In an Ar atmosphere, in a 300 mL three-neck flask, Intermediate IM-20 (10.00 g, 23.5 mmol), Pd(dba)₂ (0.41 g, 0.03 equiv, 0.8 mmol), NaOtBu (4.52 g, 2.0 equiv, 47.0 mmol), toluene (118 mL), 4-bromo-6-phenyldibenzothiophene (8.77 g, 1.1 equiv, 25.9 mmol), and tBu₃P (0.48 g, 0.1 equiv, 2.4 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound D44 (10.93 g, yield 68%).

By measuring FAB-MS, a mass number of m/z=683 was observed by molecular ion peak, thereby identifying Compound D44.

<Synthesis of Compound D85>

Polycyclic Compound D85 according to an example may be synthesized by, for example, the steps shown in Reaction Scheme 17 below:

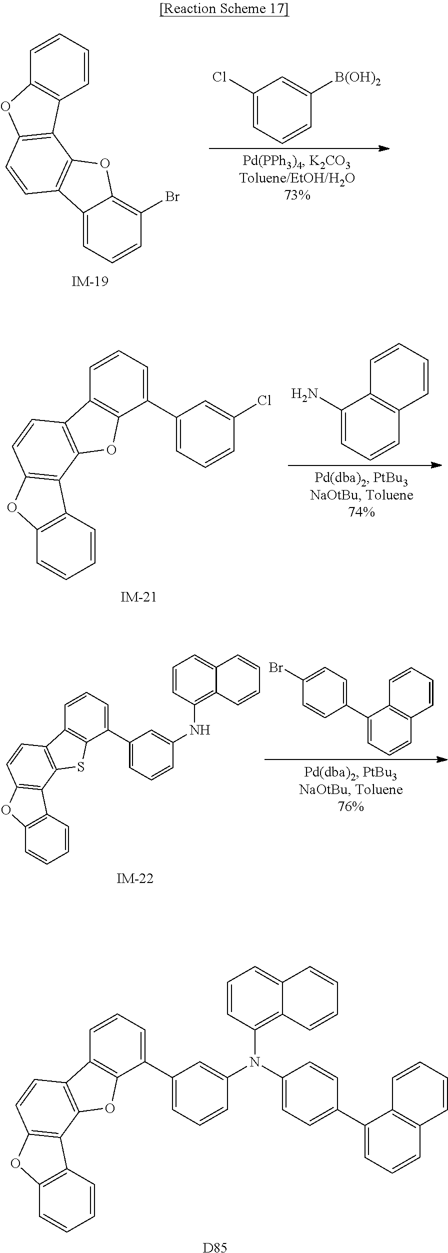

(Synthesis of Intermediate IM-21)

In an Ar atmosphere, in a 500 mL three-neck flask, Intermediate IM-19 (15.00 g, 44.5 mmol), 3-chlorophenylboronic acid (7.65 g, 1.1 equiv, 48.9 mmol), K₂CO₃ (18.45 g, 3.0 equiv, 133.5 mmol), Pd(PPh₃)₄ (2.57 g, 0.05 equiv, 2.2 mmol), and a mixed solution of toluene/ethanol/water (4/2/1, 311 mL) were sequentially added, and heated and stirred at about 80° C. After air cooling to room temperature, the reaction solution was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-21 (11.98 g, yield 73%).

By measuring FAB-MS, a mass number of m/z=368 was observed by molecular ion peak, thereby identifying Intermediate IM-21.

(Synthesis of Intermediate IM-22)

In an Ar atmosphere, in a 500 mL three-neck flask, Intermediate IM-21 (13.00 g, 35.2 mmol), Pd(dba)₂ (0.61 g, 0.03 equiv, 1.1 mmol), NaOtBu (3.39 g, 1.0 equiv, 35.2 mmol), toluene (176 mL), naphthalen-1-amine (5.55 g, 1.1 equiv, 38.8 mmol), and tBu₃P (0.71 g, 0.1 equiv, 3.5 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-22 (12.82 g, yield 74%).

By measuring FAB-MS, a mass number of m/z=491 was observed by molecular ion peak, thereby identifying Intermediate IM-22.

(Synthesis of Compound D85)

In an Ar atmosphere, in a 300 mL three-neck flask, Intermediate IM-22 (10.00 g, 20.3 mmol), Pd(dba)₂ (0.35 g, 0.03 equiv, 0.6 mmol), NaOtBu (3.91 g, 2.0 equiv, 40.7 mmol), toluene (102 mL), 1-(4-bromophenyl)naphthalene (6.34 g, 1.1 equiv, 22.4 mmol), and tBu₃P (0.41 g, 0.1 equiv, 2.0 mmol) were sequentially added, and heated and stirred under reflux. After air cooling to room temperature, the organic layer was fractionated by adding water to the reaction solvent. The organic layers were further extracted by further toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound D85 (10.48 g, yield 76%).

By measuring FAB-MS, a mass number of m/z=677 was observed by molecular ion peak, thereby identifying Compound D85.

<Synthesis of Compound D108>

Polycyclic Compound D108 according to an example may be synthesized by, for example, the steps shown in Reaction Scheme 18 below:

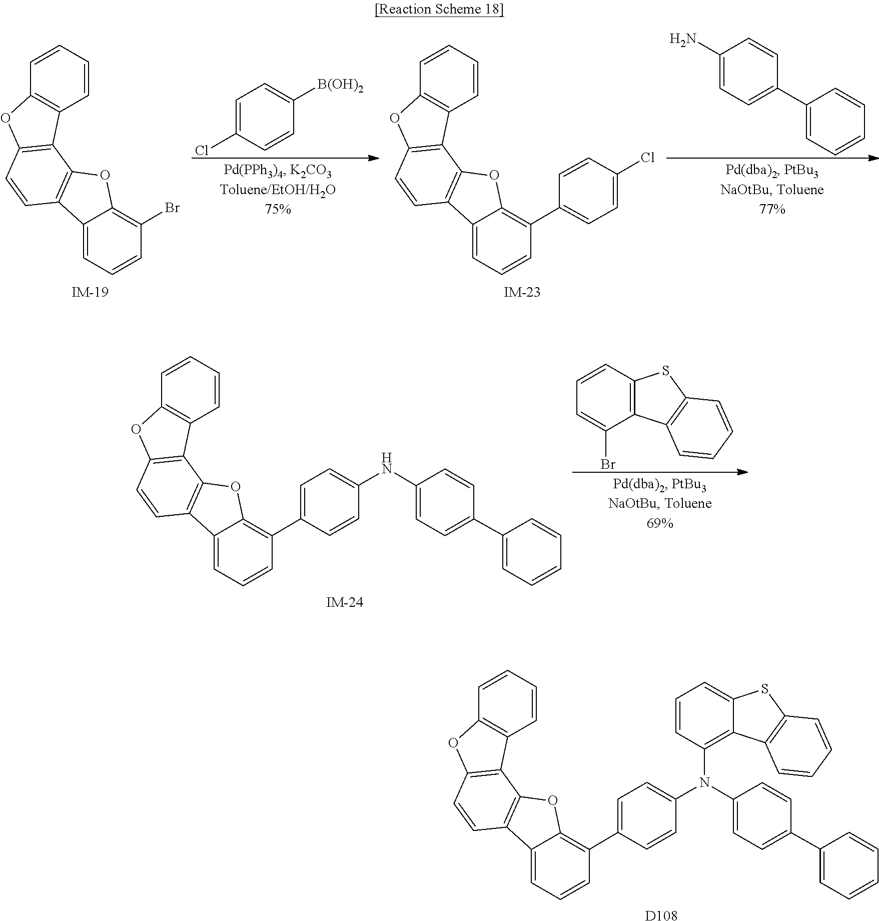

(Synthesis of intermediate IM-23)

In an Ar atmosphere, in a 500 mL three-neck flask, Intermediate IM-19 (15.00 g, 44.5 mmol), 4-chlorophenylboronic acid (7.65 g, 1.1 equiv, 48.9 mmol), K₂CO₃ (18.45 g, 3.0 equiv, 133.5 mmol), Pd(PPh₃)₄ (2.57 g, 0.05 equiv, 2.2 mmol), and a mixed solution of toluene/ethanol/water (4/2/1, 311 mL) were sequentially added, and heated and stirred at about 80° C. After air cooling to room temperature, the reaction solution was extracted with toluene to obtain organic layers. A water layer was removed the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-23 (12.31 g, yield 75%).

By measuring FAB-MS, a mass number of m/z=368 was observed by molecular ion peak, thereby identifying Intermediate IM-23.

(Synthesis of Intermediate IM-24)

In an Ar atmosphere, in a 500 mL three-neck flask, Intermediate IM-23 (13.00 g, 35.2 mmol), Pd(dba)₂ (0.61 g, 0.03 equiv, 1.1 mmol), NaOtBu (3.39 g, 1.0 equiv, 35.2 mmol), toluene (176 mL), 4-biphenylamine (6.56 g, 1.1 equiv, 38.8 mmol), and tBu₃P (0.71 g, 0.1 equiv, 3.5 mmol) were sequentially added, and heated and stirred under reflux. After air cooling to room temperature, organic layers were separated and obtained by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-24 (13.61 g, yield 77%).

By measuring FAB-MS, a mass number of m/z=501 was observed by molecular ion peak, thereby identifying Intermediate IM-24.

(Synthesis of Compound D108)

In an Ar atmosphere, in a 300 mL three-neck flask, Intermediate IM-24 (10.00 g, 36.8 mmol), Pd(dba)₂ (0.34 g, 0.03 equiv, 0.6 mmol), NaOtBu (3.83 g, 2.0 equiv, 36.8 mmol), toluene (100 mL), 1-bromodibenzothiophene (5.77 g, 1.1 equiv, 21.9 mmol), and tBu₃P (0.40 g, 0.1 equiv, 2.0 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound D108 (9.41 g, yield 69%).

By measuring FAB-MS, a mass number of m/z=683 was observed by molecular ion peak, thereby identifying Compound D108.

<Synthesis of Compound D137>

Polycyclic Compound D137 according to an example may be synthesized by, for example, the steps shown in Reaction Scheme 19 below:

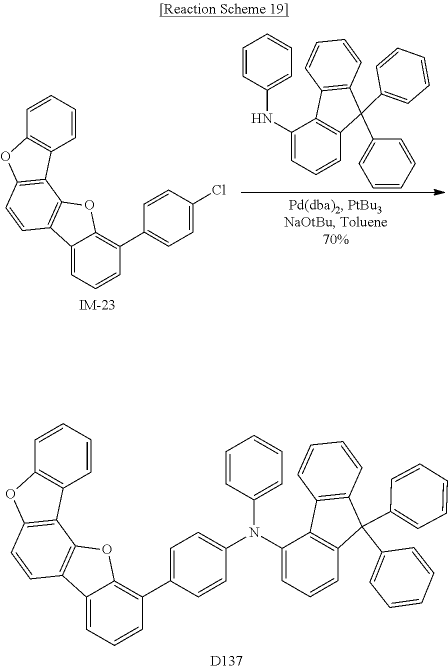

In an Ar atmosphere, in a 300 mL three-neck flask, Intermediate IM-23 (13.00 g, 35.2 mmol), Pd(dba)₂ (0.61 g, 0.03 equiv, 1.1 mmol), NaOtBu (6.77 g, 2.0 equiv, 70.5 mmol), toluene (176 mL), N,9,9-triphenyl-9H-fluoren-4-amine (15.88 g, 1.1 equiv, 38.8 mmol), and tBu₃P (0.71 g, 0.1 equiv, 3.5 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound D137 (18.30 g, yield 70%).

By measuring FAB-MS, a mass number of m/z=741 was observed by molecular ion peak, thereby identifying Compound D137.

<Synthesis of Compound D146>

Polycyclic Compound D146 according to an example may be synthesized by, for example, the steps shown in Reaction Scheme 20 below:

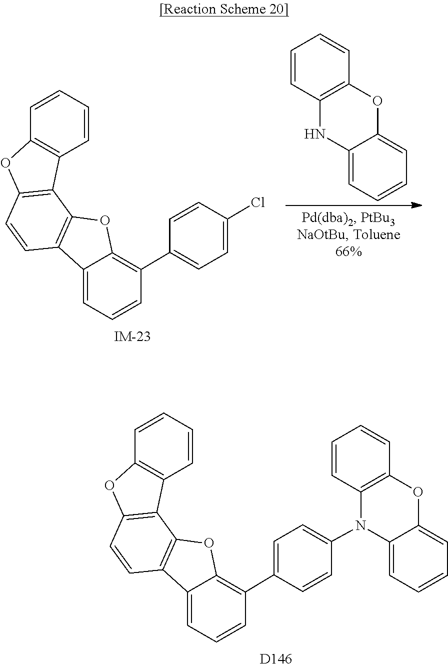

In an Ar atmosphere, in a 300 mL three-neck flask, Intermediate IM-23 (13.00 g, 36.8 mmol), Pd(dba)₂ (0.61 g, 0.03 equiv, 1.1 mmol), NaOtBu (6.77 g, 2.0 equiv, 36.8 mmol), toluene (176 mL), 10H-phenoxazine (7.10 g, 1.1 equiv, 38.8 mmol), and tBu₃P (0.71 g, 0.1 equiv, 3.5 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound D146 (11.99 g, yield 66%).

By measuring FAB-MS, a mass number of m/z=515 was observed by molecular ion peak, thereby identifying Compound D146.

<Synthesis of Compound E144>

Polycyclic Compound E144 according to an example may be synthesized by, for example, the steps shown in Reaction Scheme 21 below:

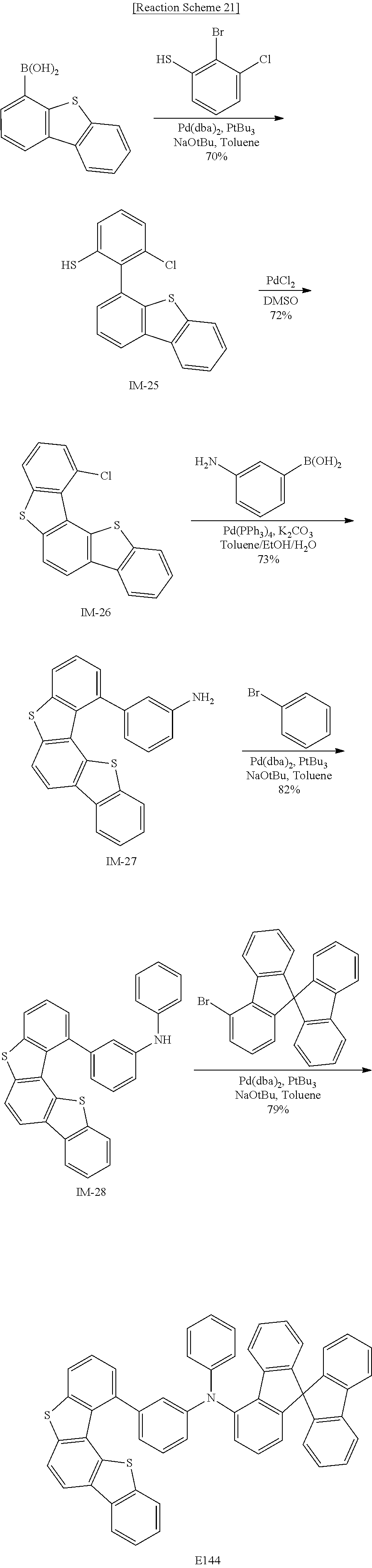

(Synthesis of Intermediate IM-25)

In an Ar atmosphere, in a 2000 mL three-neck flask, 4-dibenzothiopheneboronic acid (30.00 g, 131.5 mmol), 2-bromo-3-chlorobenzenethiol (32.34 g, 1.1 equiv, 144.7 mmol), K₂CO₃ (54.54 g, 3.0 equiv, 394.6 mmol), Pd(PPh₃)₄ (7.60 g, 0.05 equiv, 6.6 mmol), and a mixed solution of toluene/ethanol/water (4/2/1, 920 mL) were sequentially added, and heated and stirred at about 80° C. After air cooling to room temperature, the reaction solution was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-25 (30.10 g, yield 70%).

By measuring FAB-MS, a mass number of m/z=326 was observed by molecular ion peak, thereby identifying Intermediate IM-25.

(Synthesis of Intermediate IM-26)

In an Ar atmosphere, in a 1000 mL three-neck flask, Intermediate IM-25 (25.00 g, 76.5 mmol), PdCl₂ (0.68 g, 0.05 equiv, 3.8 mmol) and DMSO (510 mL) were sequentially added, and heated and stirred at about 140° C. After air cooling to room temperature, organic layers were fractionated by adding water to the reaction solvent. The organic layer was further extracted by adding CH₂Cl₂ to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-26 (17.89 g, yield 72%).

By measuring FAB-MS, a mass number of m/z=324 was observed by molecular ion peak, thereby identifying Intermediate IM-26.

(Synthesis of Intermediate IM-27)

In an Ar atmosphere, in a 1000 mL three-neck flask, Intermediate IM-26 (15.00 g, 46.2 mmol), (3-aminophenyl)boronic acid (6.96 g, 1.1 equiv, 50.8 mmol), K₂CO₃ (19.15 g, 3.0 equiv, 138.5 mmol), Pd(PPh₃)₄ (2.67 g, 0.05 equiv, 2.3 mmol), and a mixed solution of toluene/ethanol/water (4/2/1, 323 mL) were sequentially added, and heated and stirred at about 80° C. After air cooling to room temperature, the reaction solution was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-27 (12.86 g, yield 73%).

By measuring FAB-MS, a mass number of m/z=381 was observed by molecular ion peak, thereby identifying Intermediate IM-27.

(Synthesis of Intermediate IM-28)

In an Ar atmosphere, in a 500 mL three-neck flask, Intermediate IM-27 (10.00 g, 26.2 mmol), Pd(dba)₂ (0.45 g, 0.03 equiv, 0.78 mmol), NaOtBu (2.52 g, 1.0 equiv, 26.2 mmol), toluene (132 mL), bromobenzene (4.53 g, 1.1 equiv, 28.8 mmol), and tBu₃P (0.53 g, 0.1 equiv, 2.6 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-28 (9.84 g, yield 82%).

By measuring FAB-MS, a mass number of m/z=457 was observed by molecular ion peak, thereby identifying Intermediate IM-28.

(Synthesis of Compound E144)

In an Ar atmosphere, in a 300 mL three-neck flask, Intermediate IM-28 (8.00 g, 17.5 mmol), Pd(dba)₂ (0.30 g, 0.03 equiv, 0.5 mmol), NaOtBu (3.36 g, 2.0 equiv, 35.0 mmol), toluene (87 mL), 4-bromo-9,9′-spirobi[fluorene] (7.60 g, 1.1 equiv, 19.2 mmol), and tBu₃P (0.35 g, 0.1 equiv, 1.7 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound E144 (10.66 g, yield 79%).

By measuring FAB-MS, a mass number of m/z=772 was observed by molecular ion peak, thereby identifying Compound E144.

<Synthesis of Compound F150>

Polycyclic Compound F150 according to an example may be synthesized by, for example, the steps shown in Reaction Scheme 22 below:

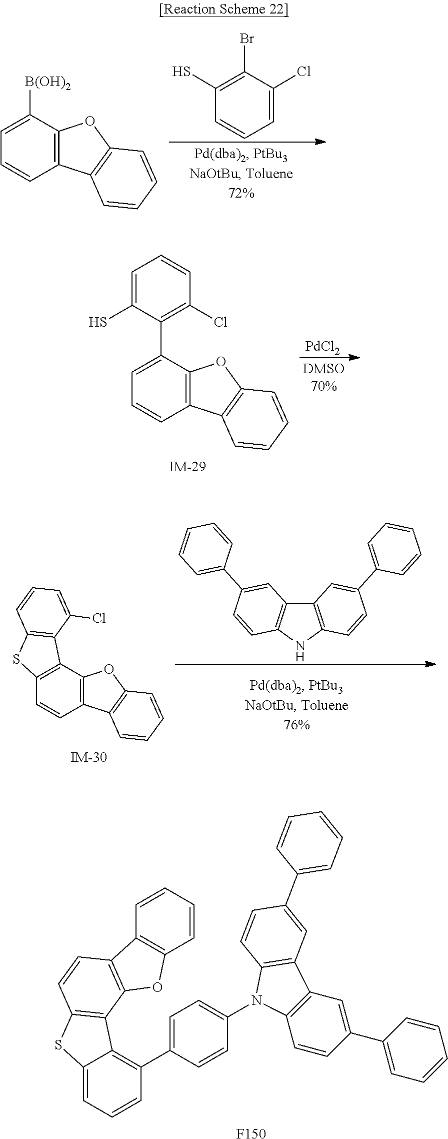

(Synthesis of Intermediate IM-29)

In an Ar atmosphere, in a 2000 mL three-neck flask, 4-dibenzofuranboronic acid (30.00 g, 141.5 mmol), 2-bromo-3-chlorobenzenethiol (34.79 g, 1.1 equiv, 155.6 mmol), K₂CO₃ (58.67 g, 3.0 equiv, 424.5 mmol), Pd(PPh₃)₄ (8.18 g, 0.05 equiv, 7.1 mmol), and a mixed solution of toluene/ethanol/water (4/2/1, 990 mL) were sequentially added, and heated and stirred at about 80° C. After air cooling to room temperature, the reaction solution was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-29 (31.66 g, yield 72%).

By measuring FAB-MS, a mass number of m/z=310 was observed by molecular ion peak, thereby identifying Intermediate IM-29.

(Synthesis of Intermediate IM-30)

In an Ar atmosphere, in a 1000 mL three-neck flask, Intermediate IM-29 (25.00 g, 80.4 mmol), PdCl₂ (0.71 g, 0.05 equiv, 4.0 mmol) and DMSO (536 mL) were sequentially added, and heated and stirred at about 140° C. After air cooling to room temperature, organic layers were fractionated by adding water to the reaction solvent. The organic layer was further extracted by adding CH₂Cl₂ to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-30 (17.39 g, yield 70%).

By measuring FAB-MS, a mass number of m/z=308 was observed by molecular ion peak, thereby identifying Intermediate IM-30.

(Synthesis of Compound F150)

In an Ar atmosphere, in a 300 mL three-neck flask, Intermediate IM-30 (8.00 g, 25.9 mmol), Pd(dba)₂ (0.45 g, 0.03 equiv, 0.8 mmol), NaOtBu (4.80 g, 2.0 equiv, 51.8 mmol), toluene (130 mL), 3,6-diphenyl-9H-carbazole (11.27 g, 1.1 equiv, 28.5 mmol), and tBu₃P (0.52 g, 0.1 equiv, 2.6 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound F150 (13.15 g, yield 76%).

By measuring FAB-MS, a mass number of m/z=667 was observed by molecular ion peak, thereby identifying Compound F150.

<Synthesis of Compound G19>

Polycyclic Compound G19 according to an example may be synthesized by, for example, the steps shown in Reaction Scheme 23 below:

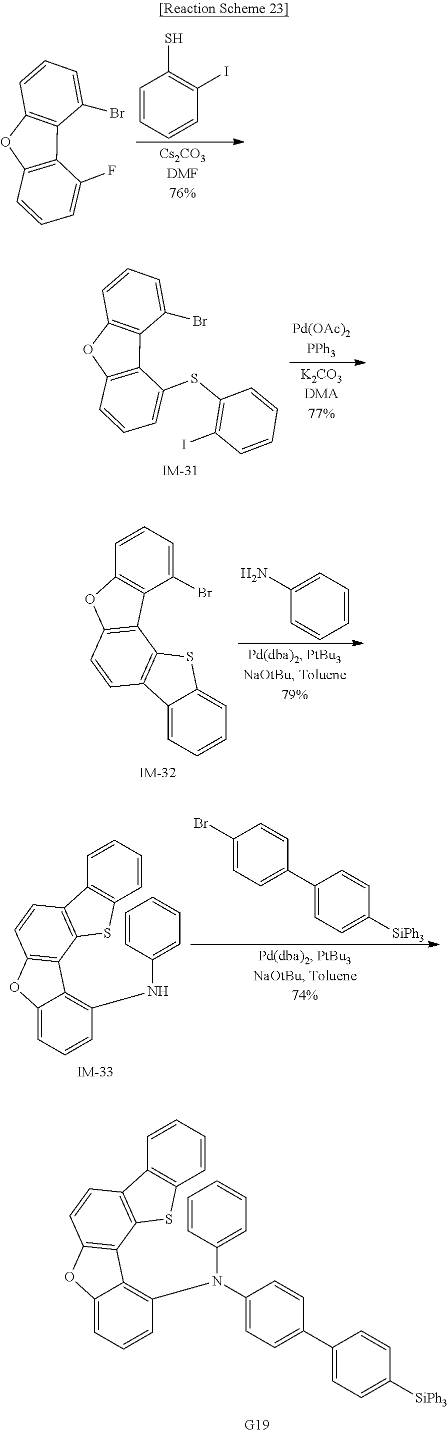

(Synthesis of Intermediate IM-31)

In an Ar atmosphere, in a 1000 mL three-neck flask, 2-iodobenzenethiol (20.00 g, 84.7 mmol), 1-bromo-9-fluorodibenzofuran (26.95 g, 1.2 equiv, 101.7 mmol), Cs₂CO₃ (55.21 g, 2.0 equiv, 169.4 mmol) and DMF (423 mL) were sequentially added, and heated and stirred at about 110° C. After air cooling to room temperature, water was added to the reaction solution, and the reaction solution was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-31 (30.98 g, yield 76%).

By measuring FAB-MS, a mass number of m/z=481 was observed by molecular ion peak, thereby identifying Intermediate IM-31.

(Synthesis of Intermediate IM-32)

In an Ar atmosphere, in a 500 mL three-neck flask, Intermediate IM-31 (25.00 g, 52.0 mmol), Pd(OAc)₂ (0.58 g, 0.05 equiv, 2.6 mmol), K₂CO₃ (10.77 g, 1.5 equiv, 77.9 mmol), PPh₃ (1.36 g, 0.10 equiv, 5.2 mmol), and DMA (208 mL) were sequentially added, and heated and stirred at about 140° C. After air cooling to room temperature, water was added to the reaction solvent, and the reaction solvent was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-32 (12.93 g, yield 77%).

By measuring FAB-MS, a mass number of m/z=323 was observed by molecular ion peak, thereby identifying Intermediate IM-32.

(Synthesis of Intermediate IM-33)

In an Ar atmosphere, in a 500 mL three-neck flask, Intermediate IM-32 (10.00 g, 30.9 mmol), Pd(dba)₂ (0.53 g, 0.03 equiv, 0.9 mmol), NaOtBu (2.97 g, 1.0 equiv, 30.9 mmol), toluene (155 mL), aniline (3.17 g, 1.1 equiv, 34.0 mmol), and tBu₃P (0.63 g, 0.1 equiv, 2.7 mmol) were sequentially added, and heated and stirred under reflux. After air cooling to room temperature, organic layers were fractionated by adding water to the reaction solvent. The organic layers were further extracted by further toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-33 (8.93 g, yield 79%).

By measuring FAB-MS, a mass number of m/z=365 was observed by molecular ion peak, thereby identifying Intermediate IM-33.

(Synthesis of Compound G19)

In an Ar atmosphere, in a 300 mL three-neck flask, Intermediate IM-33 (10.00 g, 30.9 mmol), Pd(dba)₂ (0.47 g, 0.03 equiv, 0.8 mmol), NaOtBu (5.26 g, 2.0 equiv, 54.7 mmol), toluene (136 mL), (1,1′-biphenyl)-4-yl]triphenylsilane (14.79 g, 1.1 equiv, 30.1 mmol), and tBu₃P (0.55 g, 0.1 equiv, 2.7 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound G19 (15.71 g, yield 74%).

By measuring FAB-MS, a mass number of m/z=776 was observed by molecular ion peak, thereby identifying Compound G19.

<Synthesis of Compound H130>

Polycyclic Compound H130 according to an example may be synthesized by, for example, the steps shown in Reaction Scheme 24 below:

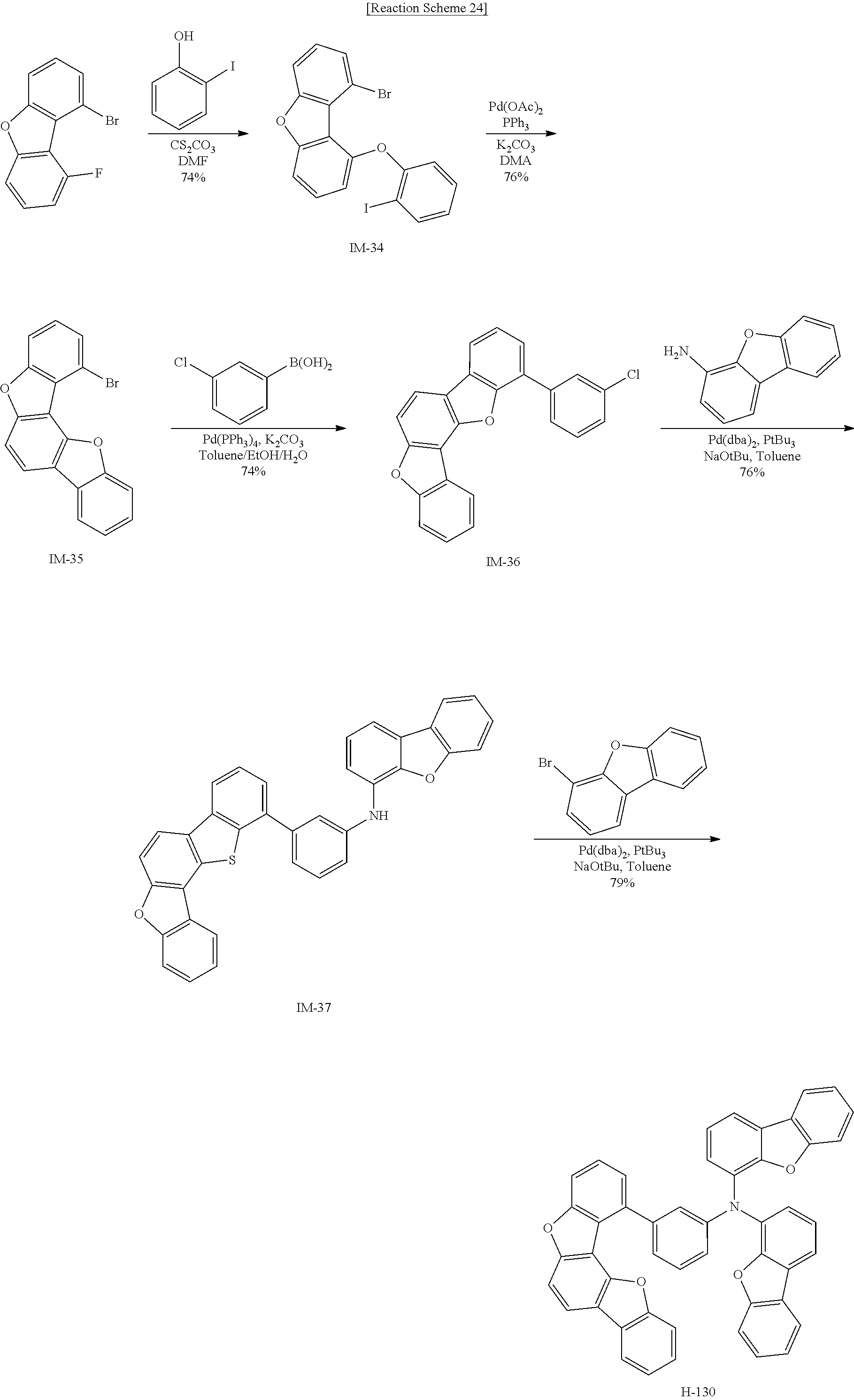

(Synthesis of Intermediate IM-34)

In an Ar atmosphere, in a 1000 mL three-neck flask, 2-iodophenol (20.00 g, 90.9 mmol), 1-bromo-9-fluorodibenzofuran (28.92 g, 1.2 equiv, 109.1 mmol), Cs₂CO₃ (59.24 g, 2.0 equiv, 181.8 mmol) and DMF (454 mL) were sequentially added, and heated and stirred at about 110° C. After air cooling to room temperature, water was added to the reaction solution, and the reaction solution was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-34 (31.29 g, yield 74%).

By measuring FAB-MS, a mass number of m/z=465 was observed by molecular ion peak, thereby identifying Intermediate IM-34.

(Synthesis of Intermediate IM-35)

In an Ar atmosphere, in a 500 mL three-neck flask, Intermediate IM-34 (25.00 g, 53.8 mmol), Pd(OAc)₂ (0.60 g, 0.05 equiv, 2.7 mmol), K₂CO₃ (11.14 g, 1.5 equiv, 80.6 mmol), PPh₃ (1.41 g, 0.10 equiv, 5.4 mmol), and DMA (215 mL) were sequentially added, and heated and stirred at about 140° C. After air cooling to room temperature, water was added to the reaction solvent, and the reaction solvent was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-35 (13.77 g, yield 76%).

By measuring FAB-MS, a mass number of m/z=337 was observed by molecular ion peak, thereby identifying Intermediate IM-35.

(Synthesis of Intermediate IM-36)

In an Ar atmosphere, in a 500 mL three-neck flask, Intermediate IM-35 (15.00 g, 44.5 mmol), 3-chlorophenylboronic acid (7.65 g, 1.1 equiv, 48.9 mmol), K₂CO₃ (18.45 g, 3.0 equiv, 133.5 mmol), Pd(PPh₃)₄ (2.57 g, 0.05 equiv, 2.2 mmol), and a mixed solution of toluene/ethanol/water (4/2/1, 311 mL) were sequentially added, and heated and stirred at about 80° C. After air cooling to room temperature, the reaction solution was extracted with toluene to obtain organic layers. A water layer was removed, the organic layers were washed with saline, and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-36 (12.14 g, yield 74%).

By measuring FAB-MS, a mass number of m/z=368 was observed by molecular ion peak, thereby identifying Intermediate IM-36.

(Synthesis of Intermediate IM-37)

In an Ar atmosphere, in a 500 mL three-neck flask, Intermediate IM-36 (10.00 g, 27.1 mmol), Pd(dba)₂ (0.47 g, 0.03 equiv, 0.8 mmol), NaOtBu (2.61 g, 1.0 equiv, 27.1 mmol), toluene (135 mL), 4-aminodibenzofuran (5.46 g, 1.1 equiv, 29.8 mmol), and tBu₃P (0.55 g, 0.1 equiv, 2.7 mmol) were sequentially added, and heated and stirred under reflux. After air cooling to room temperature, organic layers were fractionated by adding water to the reaction solvent. The organic layers were further extracted by adding toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain Intermediate IM-37 (10.95 g, yield 76%).

By measuring FAB-MS, a mass number of m/z=531 was observed by molecular ion peak, thereby identifying Intermediate IM-37.

(Synthesis of Compound H130)

In an Ar atmosphere, in a 300 mL three-neck flask, Intermediate IM-36 (10.00 g, 18.8 mmol), Pd(dba)₂ (0.32 g, 0.03 equiv, 0.6 mmol), NaOtBu (3.62 g, 2.0 equiv, 37.6 mmol), toluene (94 mL), 4-bromodibenzofuran (5.53 g, 1.1 equiv, 20.7 mmol), and tBu₃P (0.38 g, 0.1 equiv, 1.9 mmol) were sequentially added, and heated and stirred under reflux. After air cooling to room temperature, organic layers were fractionated by adding water to the reaction solvent. The organic layers were further extracted by further toluene to a water layer, and the combined organic layers were washed with saline and dried over MgSO₄. MgSO₄ was filtered off and the organic layers were concentrated, and the resulting crude product was purified by silica gel column chromatography (using a mixture solvent of hexane and toluene as an eluent) to obtain solid Compound H130 (10.13 g, yield 79%).

By measuring FAB-MS, a mass number of m/z=681 was observed by molecular ion peak, thereby identifying Compound H130.

2. Manufacture and Evaluation of Light Emitting Device

(Manufacture of Light Emitting Device)

The light emitting device of an embodiment including the polycyclic compound of an embodiment in a hole transport layer was manufactured as follows. The polycyclic compounds of Compounds A6, A26, A69, A111, A141, B12, B45, B82, B114, B149, C27, C49, C113, C138, C159, D44, D85, D108, D137, D146, E144, F150, G19, and H130 as described above were used as hole transport layer materials to manufacture the light emitting devices of Examples 1 to 24, respectively. Comparative Example Compounds R1 to R22 were used as hole transport layer materials to manufacture the light emitting devices of Comparative Examples 1 to 22, respectively.

Compounds used in the hole transport layers in Examples 1 to 24 and Comparative Examples 1 to 22 are shown as follows.

(Example Compounds Used to Manufacture Devices)

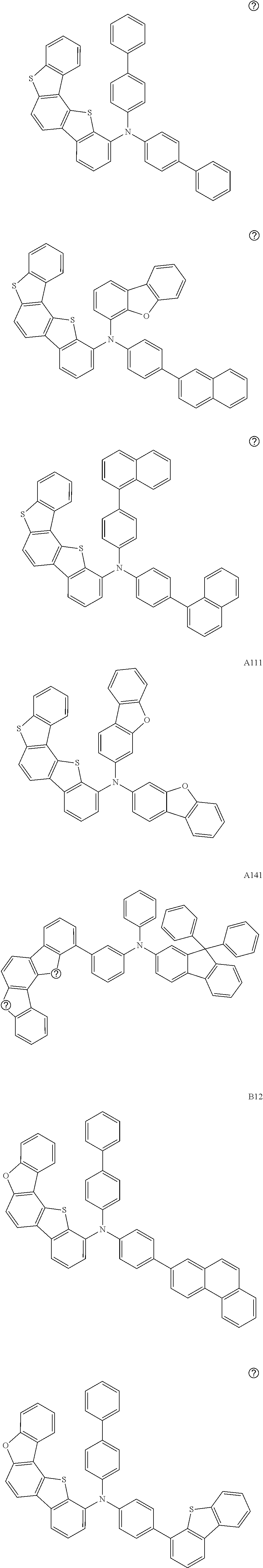

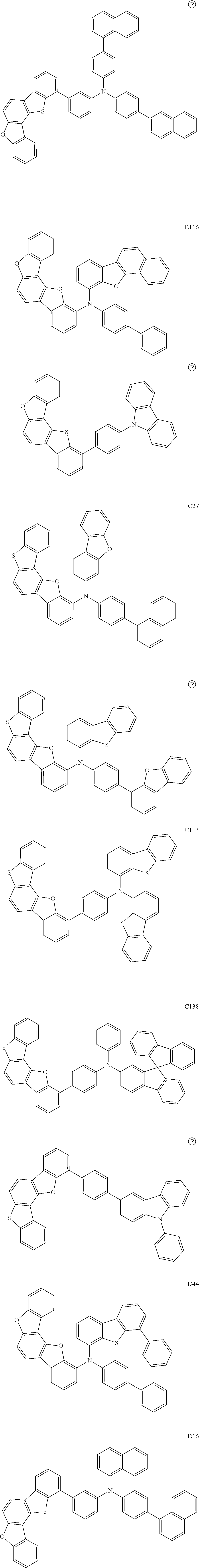

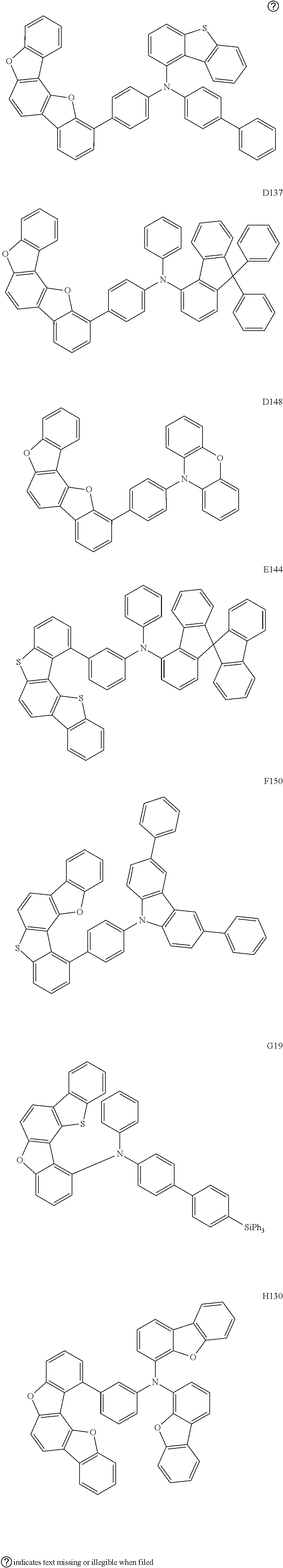

(Comparative Example Compounds Used to Manufacture Devices)

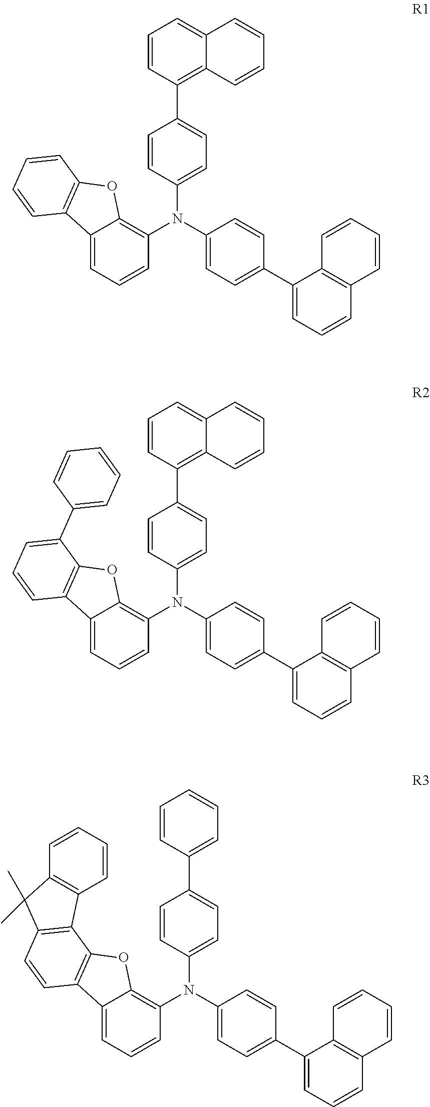 [SMILES: c1ccc2c(-c3ccc(N(c4ccc(-c5cccc6ccccc56)cc4)c4cccc5c4oc4ccccc45)cc3)cccc2c1 ; c1ccc(-c2cccc3c2oc2c(N(c4ccc(-c5cccc6ccccc56)cc4)c4ccc(-c5cccc6ccccc56)cc4)cccc23)cc1 ; CC1(C)c2ccccc2-c2c1ccc1c2oc2c(N(c3ccc(-c4ccccc4)cc3)c3ccc(-c4cccc5ccccc45)cc3)cccc21]

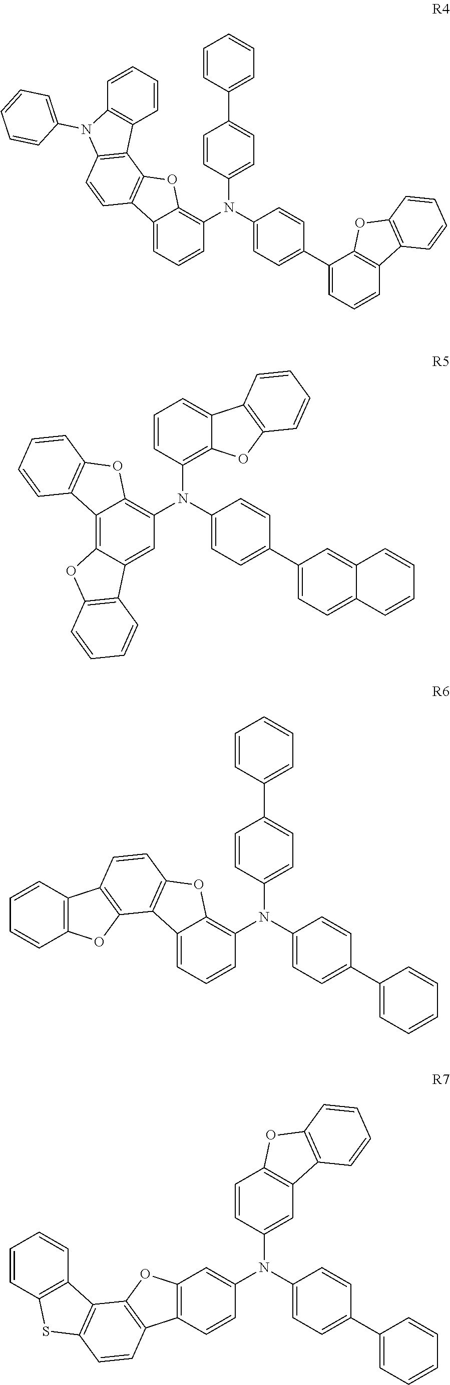 [SMILES: c1ccc(-c2ccc(N(c3ccc(-c4cccc5c4oc4ccccc45)cc3)c3cccc4c3oc3c4ccc4c3c3ccccc3n4-c3ccccc3)cc2)cc1 ; c1ccc2cc(-c3ccc(N(c4cccc5c4oc4ccccc45)c4cc5c6ccccc6oc5c5c4oc4ccccc45)cc3)ccc2c1 ; c1ccc(-c2ccc(N(c3ccc(-c4ccccc4)cc3)c3cccc4c3oc3ccc5c6ccccc6oc5c34)cc2)cc1 ; c1ccc(-c2ccc(N(c3ccc4c(c3)oc3c4ccc4sc5ccccc5c43)c3ccc4oc5ccccc5c4c3)cc2)cc1]

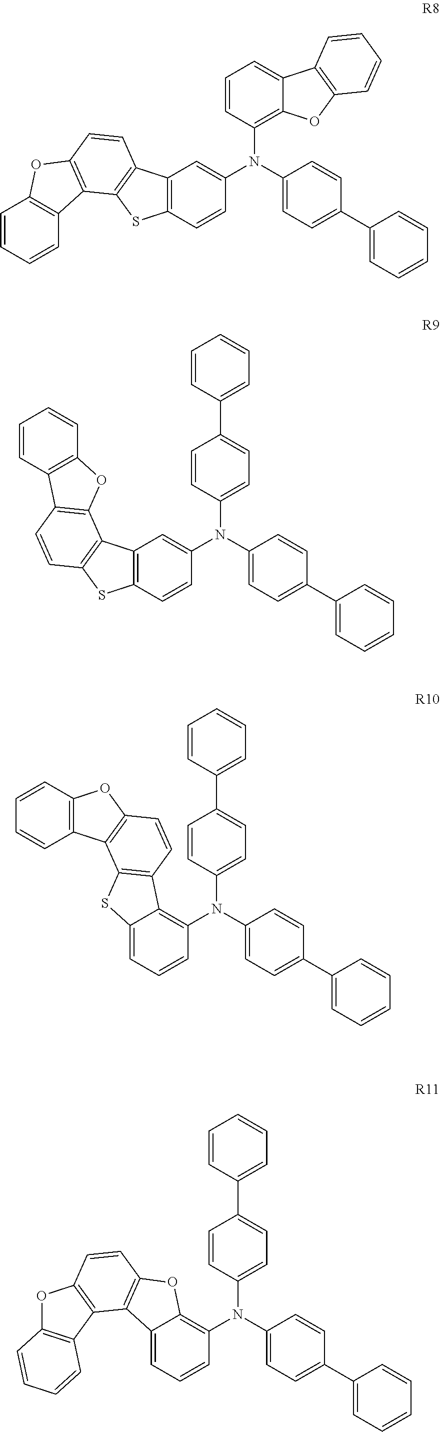 [SMILES: c1ccc(-c2ccc(N(c3ccc4sc5c(ccc6oc7ccccc7c65)c4c3)c3cccc4c3oc3ccccc34)cc2)cc1 ; c1ccc(-c2ccc(N(c3ccc(-c4ccccc4)cc3)c3ccc4sc5ccc6c7ccccc7oc6c5c4c3)cc2)cc1 ; c1ccc(-c2ccc(N(c3ccc(-c4ccccc4)cc3)c3cccc4sc5c(ccc6oc7ccccc7c65)c34)cc2)cc1 ; c1ccc(-c2ccc(N(c3ccc(-c4ccccc4)cc3)c3cccc4c3oc3ccc5oc6ccccc6c5c34)cc2)cc1]

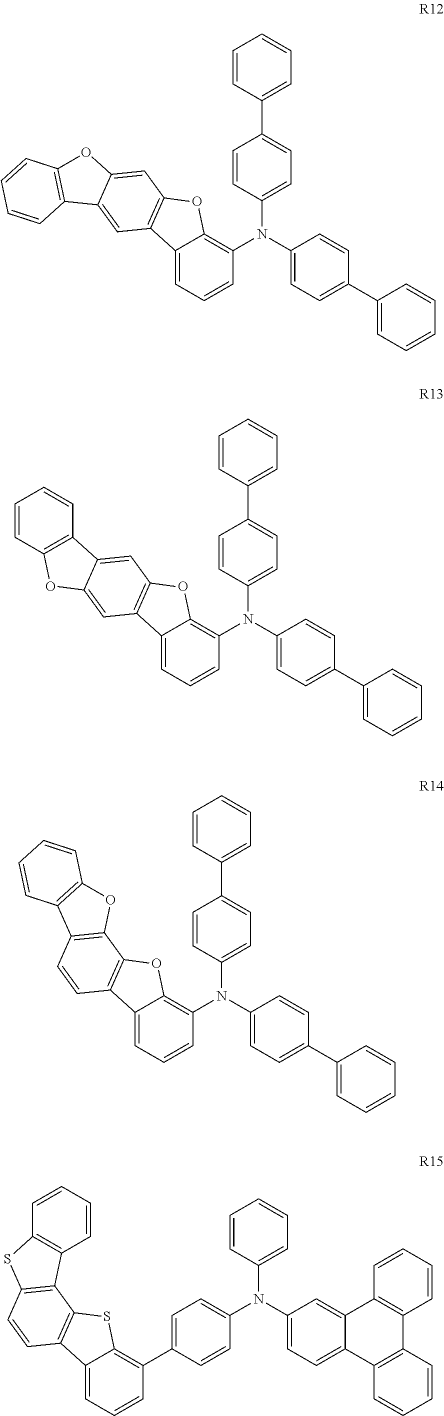 [SMILES: c1ccc(-c2ccc(N(c3ccc(-c4ccccc4)cc3)c3cccc4c3oc3cc5oc6ccccc6c5cc34)cc2)cc1 ; c1ccc(-c2ccc(N(c3ccc(-c4ccccc4)cc3)c3cccc4c3oc3cc5c(cc34)oc3ccccc35)cc2)cc1 ; c1ccc(-c2ccc(N(c3ccc(-c4ccccc4)cc3)c3cccc4c3oc3c4ccc4c5ccccc5oc43)cc2)cc1 ; c1ccc(N(c2ccc(-c3cccc4c3sc3c4ccc4sc5ccccc5c43)cc2)c2ccc3c4ccccc4c4ccccc4c3c2)cc1]

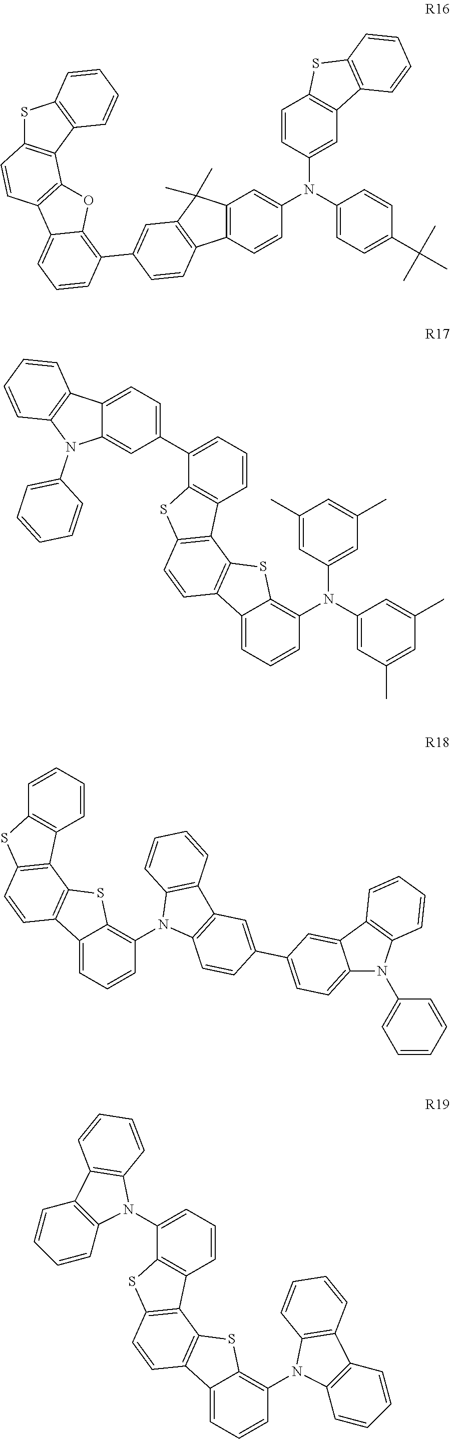 [SMILES: CC(C)(C)c1ccc(N(c2ccc3c(c2)C(C)(C)c2cc(-c4cccc5c4oc4c5ccc5sc6ccccc6c54)ccc2-3)c2ccc3sc4ccccc4c3c2)cc1 ; Cc1cc(C)cc(N(c2cc(C)cc(C)c2)c2cccc3c2sc2c3ccc3sc4c(-c5ccc6c7ccccc7n(-c7ccccc7)c6c5)cccc4c32)c1 ; c1ccc(-n2c3ccccc3c3cc(-c4ccc5c(c4)c4ccccc4n5-c4cccc5c4sc4c5ccc5sc6ccccc6c54)ccc32)cc1 ; c1cc(-n2c3ccccc3c3ccccc32)c2sc3c(ccc4sc5c(-n6c7ccccc7c7ccccc76)cccc5c43)c2c1]

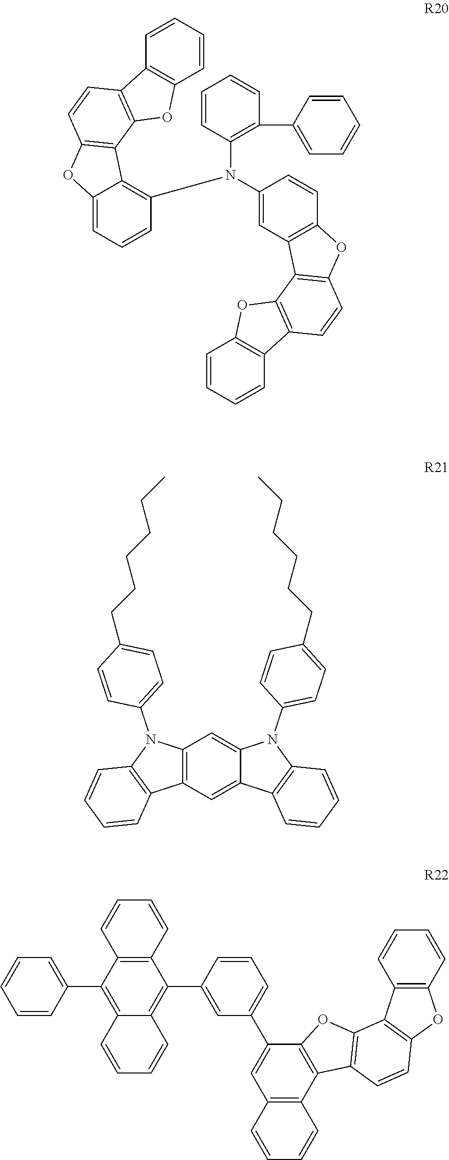 [SMILES: c1ccc(-c2ccccc2N(c2ccc3oc4ccc5c6ccccc6oc5c4c3c2)c2cccc3oc4ccc5c6ccccc6oc5c4c23)cc1 ; CCCCCCc1ccc(-n2c3ccccc3c3cc4c5ccccc5n(-c5ccc(CCCCCC)cc5)c4cc32)cc1 ; c1ccc(-c2c3ccccc3c(-c3cccc(-c4cc5ccccc5c5c4oc4c5ccc5oc6ccccc6c54)c3)c3ccccc23)cc1]

A 1500 Å-thick ITO was patterned on a glass substrate, washed with ultrapure water, and UV ozone-treated for about 10 minutes. 2-TNATA was deposited to form a 600 Å-thick hole injection layer. Example Compound or Comparative Example Compound was deposited to form a 300 Å-thick hole transport layer.

TBP was doped to ADN by 3% to form a 250 Å-thick emission layer. Alq3 was deposited to form a 250 Å-thick electron transport layer, and LiF was deposited to form a 10 Å-thick electron injection layer.

A1 was provided to form a 1000 Å-thick second electrode.

In the Examples, the hole injection layer, the hole transport layer, the emission layer, the electron transport layer, the electron injection layer, and the second electrode were formed by using a vacuum deposition apparatus.

(Evaluation of Light Emitting Device Characteristics)

Evaluation results of the light emitting devices of Examples 1 to 24 and Comparative Examples 1 to 22 are listed in Table 1. Driving voltage, luminous efficiency and a device service life of the manufactured light emitting devices are listed in comparison in Table 1. In the evaluation results of the characteristics for Examples and Comparative Examples listed in Table 1, the luminous efficiency shows an efficiency value at a current density of 10 mA/cm², and the device service life (LT50) shows a brightness half-life at 1.0 mA/cm².

Current densities, voltages and luminous efficiencies of the light emitting devices of Examples and Comparative Examples were measured in a dark room by using 2400 Series Source Meter from Keithley Instruments, Inc., CS-200, Color and Luminance Meter from Konica Minolta, Inc., PC Program LabVIEW 8.2 for the measurement from Japan National Instrument, Inc.

TABLE 1

| Device | | | | Service |
|---|---|---|---|---|
| manufacturing | Hole transport | Voltage | Efficiency | life |
| examples | layer material | (V) | (cd/A) | LT50 (h) |
| Example 1 | Example Compound A6 | 5.4 | 8.3 | 2250 |
| Example 2 | Example Compound A26 | 5.6 | 8.4 | 2200 |
| Example 3 | Example Compound A69 | 5.5 | 8.2 | 2300 |
| Example 4 | Example Compound A111 | 5.4 | 8.1 | 2400 |
| Example 5 | Example Compound A141 | 5.4 | 8.2 | 2150 |
| Example 6 | Example Compound B12 | 5.5 | 8.3 | 2300 |
| Example 7 | Example Compound B45 | 5.5 | 8.3 | 2200 |
| Example 8 | Example Compound B82 | 5.4 | 8.3 | 2200 |
| Example 9 | Example Compound B114 | 5.4 | 8.2 | 2300 |
| Example 10 | Example Compound B149 | 5.6 | 8.1 | 2350 |
| Example 11 | Example Compound C27 | 5.6 | 8.5 | 2150 |
| Example 12 | Example Compound C49 | 5.4 | 8.5 | 2100 |
| Example 13 | Example Compound C113 | 5.4 | 8.2 | 2300 |
| Example 14 | Example Compound C138 | 5.6 | 8.0 | 2250 |
| Example 15 | Example Compound C159 | 5.4 | 8.3 | 2100 |
| Example 16 | Example Compound D44 | 5.4 | 8.4 | 2200 |
| Example 17 | Example Compound D85 | 5.5 | 8.4 | 2150 |
| Example 18 | Example Compound D108 | 5.5 | 8.3 | 2150 |
| Example 19 | Example Compound D137 | 5.6 | 8.3 | 2200 |
| Example 20 | Example Compound D146 | 5.5 | 8.1 | 2250 |
| Example 21 | Example Compound E144 | 5.5 | 8.0 | 2300 |
| Example 22 | Example Compound F150 | 5.4 | 8.1 | 2250 |
| Example 23 | Example Compound G19 | 5.4 | 8.3 | 2100 |
| Example 24 | Example Compound H130 | 5.5 | 8.1 | 2200 |
| Comparative | Comparative Example | 6.0 | 7.7 | 1950 |
| Example 1 | Compound R1 | | | |
| Comparative | Comparative Example | 5.9 | 7.8 | 1950 |
| Example 2 | Compound R2 | | | |
| Comparative | Comparative Example | 6.0 | 7.4 | 1850 |
| Example 3 | Compound R3 | | | |
| Comparative | Comparative Example | 5.8 | 7.6 | 1900 |
| Example 4 | Compound R4 | | | |
| Comparative | Comparative Example | 5.6 | 7.7 | 2050 |
| Example 5 | Compound R5 | | | |
| Comparative | Comparative Example | 5.7 | 7.7 | 1950 |
| Example 6 | Compound R6 | | | |
| Comparative | Comparative Example | 5.6 | 7.6 | 2050 |
| Example 7 | Compound R7 | | | |
| Comparative | Comparative Example | 5.7 | 7.6 | 2000 |
| Example 8 | Compound R8 | | | |
| Comparative | Comparative Example | 5.7 | 7.7 | 2000 |
| Example 9 | Compound R9 | | | |
| Comparative | Comparative Example | 5.6 | 7.8 | 1950 |
| Example 10 | Compound R10 | | | |
| Comparative | Comparative Example | 5.6 | 7.8 | 1900 |
| Example 11 | Compound R11 | | | |
| Comparative | Comparative Example | 5.6 | 7.7 | 1950 |
| Example 12 | Compound R12 | | | |
| Comparative | Comparative Example | 5.6 | 7.7 | 1900 |
| Example 13 | Compound R13 | | | |
| Comparative | Comparative Example | 5.6 | 7.6 | 1950 |
| Example 14 | Compound R14 | | | |
| Comparative | Comparative Example | 6.2 | 7.4 | 1850 |
| Example 15 | Compound R15 | | | |
| Comparative | Comparative Example | 6.5 | 7.5 | 1750 |
| Example 16 | Compound R16 | | | |
| Comparative | Comparative Example | 5.8 | 7.6 | 1950 |
| Example 17 | Compound R17 | | | |
| Comparative | Comparative Example | 6.0 | 7.7 | 1850 |
| Example 18 | Compound R18 | | | |
| Comparative | Comparative Example | 6.1 | 7.6 | 1900 |
| Example 19 | Compound R19 | | | |
| Comparative | Comparative Example | 6.0 | 7.5 | 1850 |
| Example 20 | Compound R20 | | | |
| Comparative | Comparative Example | 6.3 | 6.7 | 1550 |
| Example 21 | Compound R21 | | | |
| Comparative | Comparative Example | 6.7 | 5.8 | 1400 |
| Example 22 | Compound R22 | | | |

Referring to the results of Table 1, it may be seen that Examples of the light emitting devices using the polycyclic compounds according to embodiments as hole transport layer materials exhibit low driving voltage, excellent device efficiency, and improved device service life characteristics. Referring to Table 1, it may be confirmed that the devices of Examples 1 to 24 exhibit low voltage, long service life, and high efficiency characteristics compared to those of Comparative Examples 1 to 22.

The polycyclic compound according to an example has a molecular structure, in which the benzobisdibenzoheterol moiety and the amine derivative moiety are bonded, thereby exhibiting low voltage, long service life, and high efficiency characteristics.

The benzobisdibenzoheterol skeleton of the polycyclic compound according to an example may have three structural features below, thereby contributing to high efficiency and a long service life of the light emitting device. Firstly, the end-benzene ring, which is not bonded to the amine derivative, of the benzene rings constituting the benzobisdibenzoheterol skeleton in the polycyclic compound of an example has a bond structure in which the end-benzene ring is folded towards the nitrogen atom of the amine derivative, thereby maintaining three-dimensional structure which potentially collapses the planarity of the entire molecule. The polycyclic compound of an example has reduced symmetry in the molecule to suppress crystallinity, and the quality of the film formed by using this may be improved, thereby contributing to the improvement of luminous efficiency.

Secondly, the heteroatom, which is towards the nitrogen atom of the amine derivative, of two heteroatoms contained in the benzobisdibenzoheterol skeleton stabilizes the periphery of the nitrogen atom in radical or radical cation active species, and thus the stability of the material may be improved, thereby improving the device service life.

Finally, the heteroatom, which is towards the opposite side from the nitrogen atom of the amine derivative, of two heteroatoms contained in the benzobisdibenzoheterol skeleton may promote interactions of heteroatoms between molecules, thereby improving the hole transport ability. Thus, the recombination probability of the holes and the electrons in the emission layer may be improved, thereby improving luminous efficiency.

Therefore, the polycyclic compounds according to examples may have the above-described molecular structural features, thereby having a low driving voltage characteristic and simultaneously exhibiting long service life and high efficiency characteristics.

Example Compounds used in Examples 1, 2, 6, 7, 11, 12, 16, and 23 are compounds in which the benzobisdibenzoheterol skeleton and the nitrogen atom of the amine derivative are directly bonded, and in such embodiments, luminous efficiency was improved further. This is believed that as the nitrogen atom with abundant electrons and the heteroatoms in the benzobisdibenzoheterol skeleton approach to each other, a hole transport property is improved, and thus the recombination probability of the holes and the electrons in the emission layer is improved, thereby improving luminous efficiency.

In Examples 3 to 5, 7 to 10, 13 to 15, 17 to 22, and 24, the benzobisdibenzoheterol skeleton and the nitrogen atom of the amine derivative are bonded via a linker, and in particular, the light emitting service life was improved. This is believed because Highest Occupied Molecular Orbital (HOMO) of the substituent including the amine derivative expands widely to the benzobisdibenzoheterol skeleton via a linker, and thus the stability of the radical or the radical cation active species is improved.

Comparative Example Compounds used in Comparative Examples 1 and 2 are compounds having a dibenzoheterol skeleton compared to Example Compounds, and resulted in a decrease in device efficiency compared to Examples. This is believed because the number of heteroatoms contained in the heterocycle is reduced to thus deteriorate hole transport ability, and as the injection of the holes into the emission layer is delayed, the recombination probability of the holes and the electrons is degraded.

Comparative Example Compound R3 used in Comparative Example 3 corresponds to a compound having a polycyclic heterocycle skeleton similar to Examples of the invention, but has an sp3 hybridized carbon atom part in the polycyclic heterocycle skeleton, and the device service life is reduced compared to Examples. This is believed because the sp3 hybridized carbon atom part contained in the polycyclic heterocycle skeleton is unstable under a high temperature condition, and thus decomposition occurs during deposition.

Compound R4 in Comparative Example 4 is a compound having the benzobisdibenzoheterol skeleton similar to Example Compounds, but corresponds to a compound having a nitrogen atom as a heteroatom in the benzobisdibenzoheterol skeleton. In Comparative Example Compound R4, the hole transport property becomes higher than necessary to thus lose carrier balance, resulting in a decrease in both the device efficiency and the device service life. From a comparison of Examples and Comparative Examples 1 to 4, it may be seen that it is important to choose the kinds and number of heteroatoms contained in the polycyclic heterocycle, and only the case of having the benzobisdibenzoheterol skeleton represented in Example Compounds may exhibit excellent device characteristics.

Comparative Example Compound R5 used in Comparative Example 5 is a material having the benzobisdibenzoheterol skeleton with the same condensing type as Example Compounds, but the amine derivate is substituted at the central benzene ring, to which two heteroatoms are bonded, of the benzobisdibenzoheterol skeleton, and shows a result of reducing the device efficiency compared to Examples. It is believed that if the amine derivative is substituted at the benzene ring to which two heteroatoms with a high electronegativity are bonded, the electron density of the nitrogen atom of the amine derivative is relatively reduced to delay the generation of the radical or the radical cation active species, and thus the hole transport property is reduced.

Comparative Examples used in Comparative Examples 6 to 10 are materials having the benzobisdibenzoheterol skeleton with the same condensing type as Example Compounds, but have different bonding position with the amine derivative compared to Example Compounds, and show a result of reducing both the device efficiency and the device service life compared to Examples.

Comparative Examples used in Comparative Examples 6 to 8 and 10 each represent a structure at which the amine derivative is substituted so that the end-benzene ring, which is not bonded to the amine derivative, of the benzobisdibenzoheterol skeleton is towards the opposite side from the nitrogen atom of the amine derivative. Such Comparative Examples in Comparative Examples 6 to 8 and 10 exhibit characteristics in that the planarity of the entire molecule is significantly increased to increase stacking between molecules, thereby increasing the deposition temperature of the material and reducing the layer-forming property thereof. Accordingly, Comparative Examples 6 to 8 and 10 show a result of reducing device characteristics.

As shown in Examples, in the case of compounds having the same structure as the structure in which the end-benzene ring, to which the amine derivative is not bonded, of the benzobisdibenzoheterol skeleton, is towards the nitrogen atom, the planarity of the entire molecule is eliminated due to a potential large volume derived from the benzobisdibenzoheterol skeleton, thereby suppressing the crystallinity of the material, and thus high emission characteristics may be exhibited.

Compared to Comparative Examples used in Comparative Examples 6 to 8 and 10, Comparative Example Compound R9 used in Comparative Example 9 has the structure in which the end-benzene ring, to which the amine derivative is not bonded, of the benzobisdibenzoheterol skeleton, is towards the nitrogen atom, and thus high planarity of the molecular structure is partially eliminated, but has a different bonding structure from the compounds of Examples, thereby reducing device characteristics compared to Examples.

Example Compounds exhibit high device characteristics because one between the two heteroatoms contained in the benzobisdibenzoheterol skeleton is located spatially near the nitrogen atom of the amine derivative, thereby stabilizing the vicinity of the nitrogen atom in the radical or the radical cation active species. In comparison, Comparative Example Compound R9 in Comparative Example 9 lacks an effect of stabilizing the vicinity of the nitrogen atom in the radical or the radical cation active species because both the two heteroatoms contained in the benzobisdibenzoheterol skeleton are located spatially away from the nitrogen atom. Accordingly, it is believed that Comparative Example 9 has reduced device characteristics compared to Examples.

Comparative Example Compounds in Comparative Examples 11 to 14 have the benzobisdibenzoheterol skeleton with a different condensing type from Example Compounds, and the deposition temperature is increased due to high planarity of the compounds, and thus the decomposition of the compounds occurs. Accordingly, Comparative Examples 11 to 14 show results of reducing both the device efficiency and the device service life compared to Examples.

When comparing the evaluation results of Examples and Comparative Examples 5 to 14, it may be confirmed that the condensing type of the benzobisdibenzoheterol skeleton and the bonding position of the amine derivative are important, and Example Compounds may exhibit effects of both the elimination of the planarity of the compound and the stabilization of active species due to the heteroatom, thereby exhibiting excellent device characteristics.

Comparative Example Compound R15 in Comparative Example 15 contains a triphenylene group in the molecule, stacking between molecules is increased by the influence of the triphenylene moiety having a high planarity, and thus the deposition temperature of the material is increased and the layer-forming property thereof is reduced. Accordingly, Comparative Example 15 shows a result of reducing both the device efficiency and the device service life compared to Examples.

Comparative Example Compound R16 in Comparative Example 16 contains 9,9-dimethylfluorene as a linker between the benzobisdibenzoheterol skeleton and the amine derivative. In the case of Comparative Example 16, both the device efficiency and the device service life are reduced compared to Examples. As described above, this is believed because the heteroatoms contained in the benzobisdibenzoheterol skeleton have the effect of stabilizing the vicinity of the nitrogen atom of the amine derivative in the radical or the radical cation active species, but when 9,9-dimethylfluorene lacking the stabilization for the radical is introduced as the linker, the stabilizing effect due to the heteroatom disappears, and thus the material is decomposed during the operation of the device.

Comparative Example Compound R17 contains, in the molecule, both an amine group and a carbazole group bonded to the benzobisdibenzoheterol skeleton. Comparative Example Compounds R18 and R19 each correspond to a material having two carbazole groups in the molecule. Like Comparative Example Compounds R17 to R19, in the case of containing a plurality of moieties having a nitrogen atom in the molecule, the hole transport property becomes higher than necessary to thus lose carrier balance, and thus Comparative Examples 17 to 19 result in a decrease in both the device efficiency and the device service life.

Comparative Example Compound R20 has two benzobisdibenzoheterol skeletons in the same molecule, the deposition temperature is excessively high, and thus the decomposition of the material occurs. Accordingly, Comparative Example 20 shows a result of reducing both the device efficiency and the device service life compared to Examples.

Comparative Example Compound R21 has the benzobisdibenzoheterol skeleton with a different condensing type from Example Compounds and has two nitrogen atoms as heteroatoms in the polycyclic heterocycle. Such Comparative Example Compound R21 loses carrier balance in the molecule, and thus Comparative Example 21 shows a result of reducing both the device efficiency and the device service life compared to Examples.

Comparative Example Compound R22 is a material having the benzobisdibenzoheterol skeleton similar to Example Compounds, but does not contain an amine moiety or a carbazole moiety in the same molecule, and thus the hole transport ability is not sufficient. Accordingly, Comparative Example 21 shows a result of reducing both the device efficiency and the device service life compared to Examples.

Thus, Examples 1 to 24 show results of improving both the luminous efficiency and the light emitting service life compared to Comparative Examples 1 to 22. The device efficiency and the device service life of the light emitting devices of examples may be improved simultaneously by using the polycyclic compounds of examples having the structure in which the benzobisdibenzoheterol moiety and the amine derivative moiety are bonded.

The polycyclic compound according to an example has a molecular structure, in which the benzobisdibenzoheterol moiety and the amine derivative moiety are bonded, thereby contributing to low voltage, long service life, and high efficiency characteristics of the light emitting device. The light emitting device according to an example may include the polycyclic compound of an example, thereby exhibiting long service life and high efficiency characteristics simultaneously.

The light emitting device of an embodiment may include the polycyclic compound of an embodiment in the hole transport region, thereby exhibiting high efficiency and long service life characteristics.

The polycyclic compound of an embodiment may improve luminous efficiency and a device service life of the light emitting device.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the following claims.

What is claimed is:

1. A light emitting device comprising:
a first electrode;
a second electrode disposed on the first electrode; and
at least one functional layer disposed between the first electrode and the second electrode and comprising a polycyclic compound represented by Formula 1: 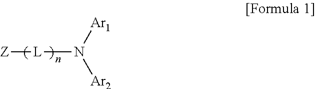
wherein in Formula 1,
n is an integer from 0 to 3,
L is a substituted or unsubstituted arylene group having 6 to 40 ring-forming carbon atoms and excluding fluorene, or a substituted or unsubstituted heteroarylene group having 2 to 40 ring-forming carbon atoms and excluding N as a ring-forming atom, or is bonded to Ar₁ or Ar₂ by using a single bond, O, S, or C(R₁)(R₂) as a linker to form a ring,
Ar₁ and Ar₂ are each independently a substituted or unsubstituted aryl group having 6 to 40 ring-forming carbon atoms and excluding triphenylene, or a substituted or unsubstituted heteroaryl group having 2 to 40 ring-forming carbon atoms, or are bonded to L or an adjacent substituent by using a single bond, O, S, or C(R₁)(R₂) as a linker to form a ring, and
R₁ and R₂ are each independently a substituted or unsubstituted alkyl group having 1 to 20 carbon atoms, or a substituted or unsubstituted aryl group having 6 to 40 ring-forming carbon atoms, and
Z is a group represented by Formula 2-1 or Formula 2-2: 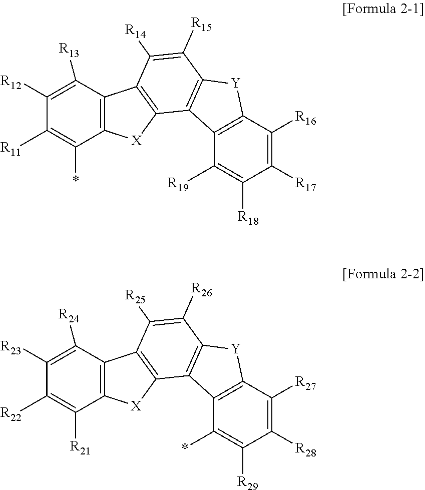
wherein in Formula 2-1 and Formula 2-2,
X and Y are each independently O or S,
R₁₁ to R₁₉ and R₂₁ to R₂₉ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, or are bonded to an adjacent group to form a ring, and
* indicates a binding site to a neighboring atom.

2. The light emitting device of claim 1, wherein
the at least one functional layer comprises:
an emission layer;
a hole transport region disposed between the first electrode and the emission layer; and
an electron transport region disposed between the emission layer and the second electrode, and
the hole transport region comprises the polycyclic compound.

3. The light emitting device of claim 2, wherein
the hole transport region comprises at least one of a hole injection layer, a hole transport layer, and an electron blocking layer, and
at least one of the hole injection layer, the hole transport layer, and the electron blocking layer comprises the polycyclic compound.

4. The light emitting device of claim 1, wherein in Formula 1, two selected from L, Ar₁, and Ar₂ are bonded to each other to form a ring.

5. The light emitting device of claim 1, wherein Formula 1 is represented by one of Formula 1-1 to Formula 1-4: 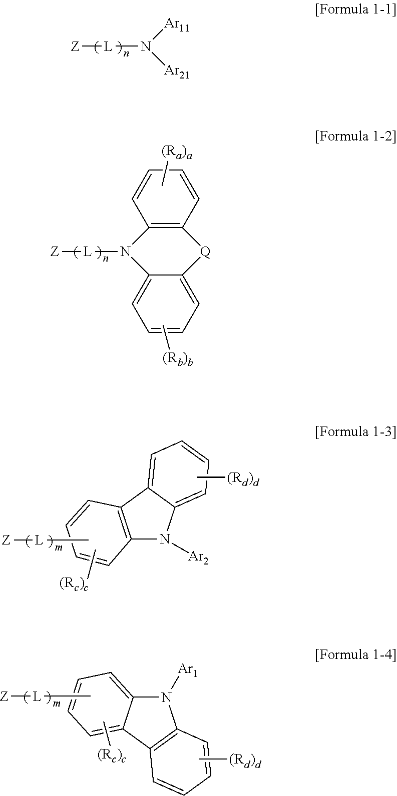
wherein in Formula 1-1
Ar₁₁ and Ar₂₁ are each independently a substituted or unsubstituted aryl group having 6 to 40 ring-forming carbon atoms and excluding triphenylene, or a substituted or unsubstituted heteroaryl group having 2 to 40 ring-forming carbon atoms and excluding N as a ring-forming atom,
in Formula 1-2,
Q is a single bond, O, S, or C(R₁)(R₂), and
a and b are each independently an integer from 0 to 4,
in Formula 1-3 and Formula 1-4,
m is an integer from 0 to 2,
c is an integer from 0 to 3,
d is an integer from 0 to 4, and
in Formula 1-2 to Formula 1-4,
Rₐ to R_{d} are each independently a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms, or bonded to an adjacent group to form an aromatic ring, and
in Formula 1-1 to Formula 1-4,
Z, L, n, R₁, R₂, Ar₁, and Ar₂ are the same as defined in connection with Formula 1.

6. The light emitting device of claim 1, wherein Formula 2-1 is represented by one of Formula 2-1A to Formula 2-1D: 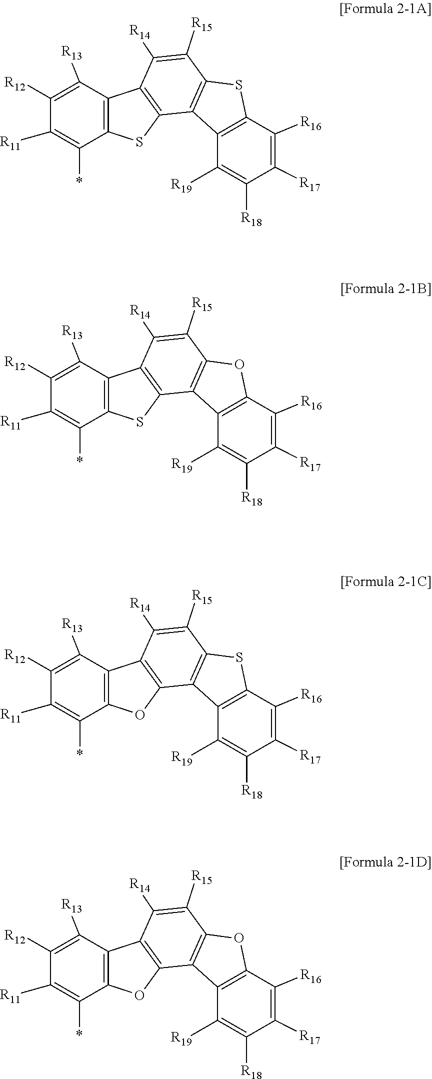
wherein in Formula 2-1A to Formula 2-1D,
R₁₁ to R₁₉ and * are the same as defined in connection with Formula 2-1.

7. The light emitting device of claim 1, wherein Formula 2-2 is represented by one of Formula 2-2A to Formula 2-2D: 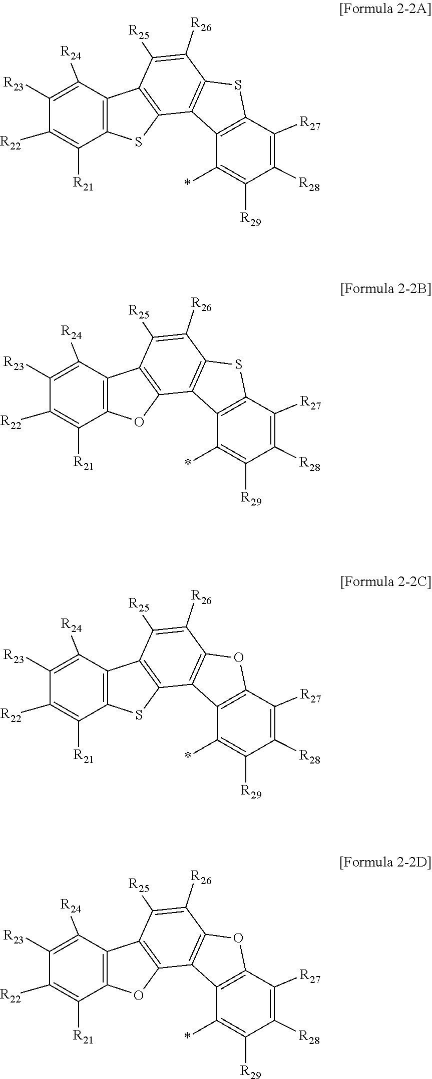
wherein in Formula 2-2A to Formula 2-2D,
R₂₁ to R₂₉ and * are the same as defined in connection with Formula 2-2.

8. The light emitting device of claim 1, wherein in Formula 1,
L is a direct linkage, a substituted or unsubstituted phenylene group, a substituted or unsubstituted divalent biphenyl group, a substituted or unsubstituted naphthalene group, a substituted or unsubstituted phenanthrene group, a substituted or unsubstituted dibenzofuranylene group, or a substituted or unsubstituted dibenzothiophenylene group.

9. The light emitting device of claim 1, wherein in Formula 2-1,
two selected from among R₁₁ to R₁₃, R₁₄ and R₁₅, or two selected from among R₁₆ to R₁₉ are bonded to each other to form a ring which is condensed with an adjacent benzene ring.

10. The light emitting device of claim 1, wherein in Formula 2-2,
two selected from among R₂₁ to R₂₃, R₂₄ and R₂₅, or two selected from among R₂₆ to R₂₉ are bonded to each other to form a ring which is condensed with an adjacent benzene ring.

11. The light emitting device of claim 1, wherein the emission layer comprises a compound represented by Formula E-1: 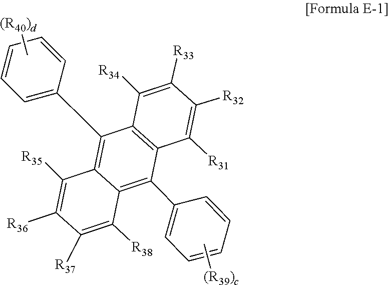
wherein in Formula E-1,
R₃₁ to R₄₀ are each independently a hydrogen atom, a deuterium atom, a halogen atom, a substituted or unsubstituted silyl group, a substituted or unsubstituted alkyl group having 1 to 10 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

12. The light emitting device of claim 1, wherein the polycyclic compound is selected from Compound Group 1A to Compound Group 1H:

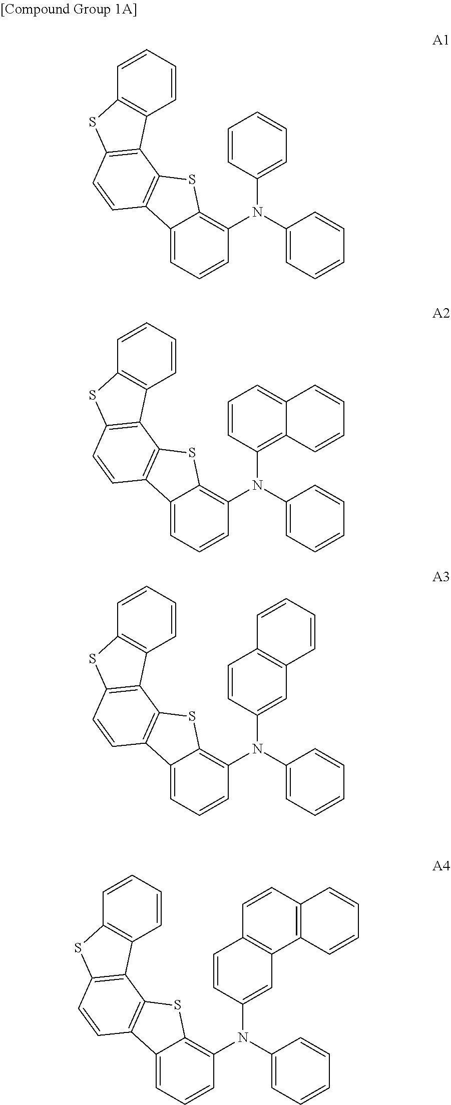 [SMILES: c1ccc(N(c2ccccc2)c2cccc3c2sc2c3ccc3sc4ccccc4c32)cc1 ; c1ccc(N(c2cccc3ccccc23)c2cccc3c2sc2c3ccc3sc4ccccc4c32)cc1 ; c1ccc(N(c2ccc3ccccc3c2)c2cccc3c2sc2c3ccc3sc4ccccc4c32)cc1 ; c1ccc(N(c2ccc3ccc4ccccc4c3c2)c2cccc3c2sc2c3ccc3sc4ccccc4c32)cc1]

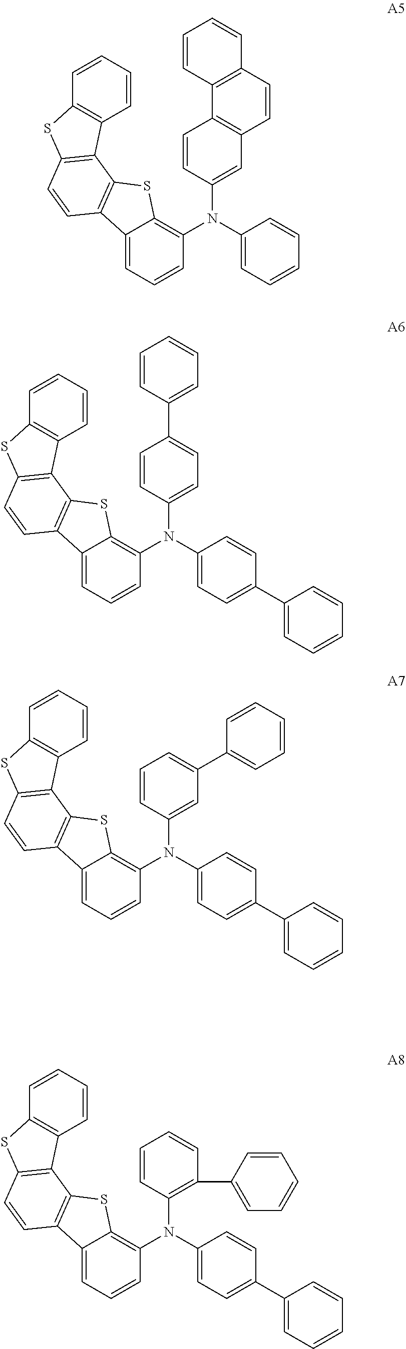 [SMILES: c1ccc(N(c2ccc3c(ccc4ccccc43)c2)c2cccc3c2sc2c3ccc3sc4ccccc4c32)cc1 ; c1ccc(-c2ccc(N(c3ccc(-c4ccccc4)cc3)c3cccc4c3sc3c4ccc4sc5ccccc5c43)cc2)cc1 ; c1ccc(-c2ccc(N(c3cccc(-c4ccccc4)c3)c3cccc4c3sc3c4ccc4sc5ccccc5c43)cc2)cc1 ; c1ccc(-c2ccc(N(c3ccccc3-c3ccccc3)c3cccc4c3sc3c4ccc4sc5ccccc5c43)cc2)cc1]

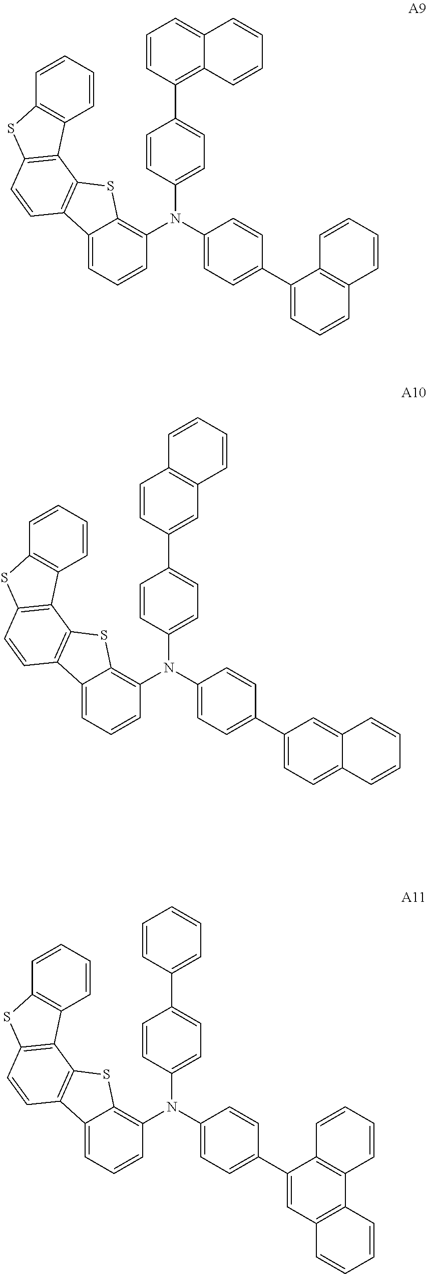 [SMILES: c1ccc2c(-c3ccc(N(c4ccc(-c5cccc6ccccc56)cc4)c4cccc5c4sc4c5ccc5sc6ccccc6c54)cc3)cccc2c1 ; c1ccc2cc(-c3ccc(N(c4ccc(-c5ccc6ccccc6c5)cc4)c4cccc5c4sc4c5ccc5sc6ccccc6c54)cc3)ccc2c1 ; c1ccc(-c2ccc(N(c3ccc(-c4cc5ccccc5c5ccccc45)cc3)c3cccc4c3sc3c4ccc4sc5ccccc5c43)cc2)cc1]

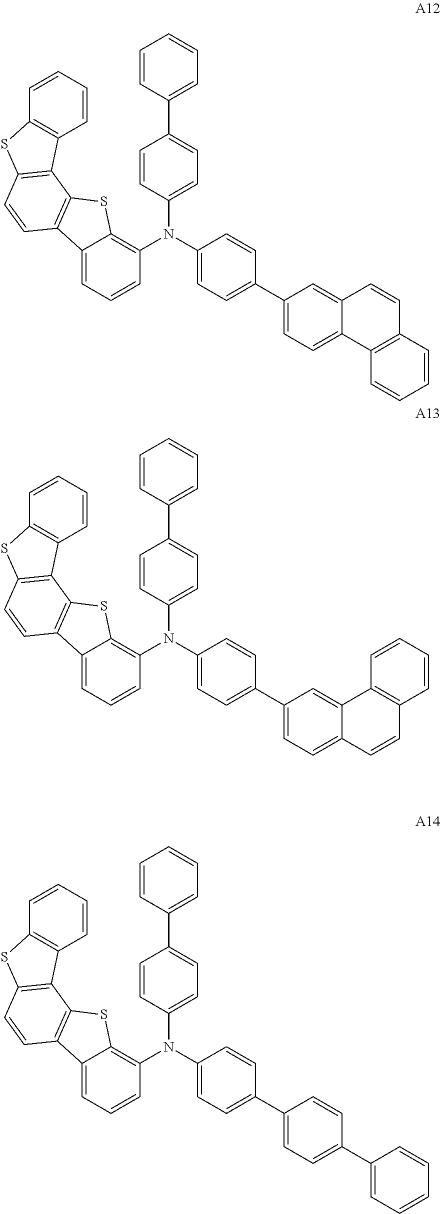 [SMILES: c1ccc(-c2ccc(N(c3ccc(-c4ccc5c(ccc6ccccc65)c4)cc3)c3cccc4c3sc3c4ccc4sc5ccccc5c43)cc2)cc1 ; c1ccc(-c2ccc(N(c3ccc(-c4ccc5ccc6ccccc6c5c4)cc3)c3cccc4c3sc3c4ccc4sc5ccccc5c43)cc2)cc1 ; c1ccc(-c2ccc(-c3ccc(N(c4ccc(-c5ccccc5)cc4)c4cccc5c4sc4c5ccc5sc6ccccc6c54)cc3)cc2)cc1]

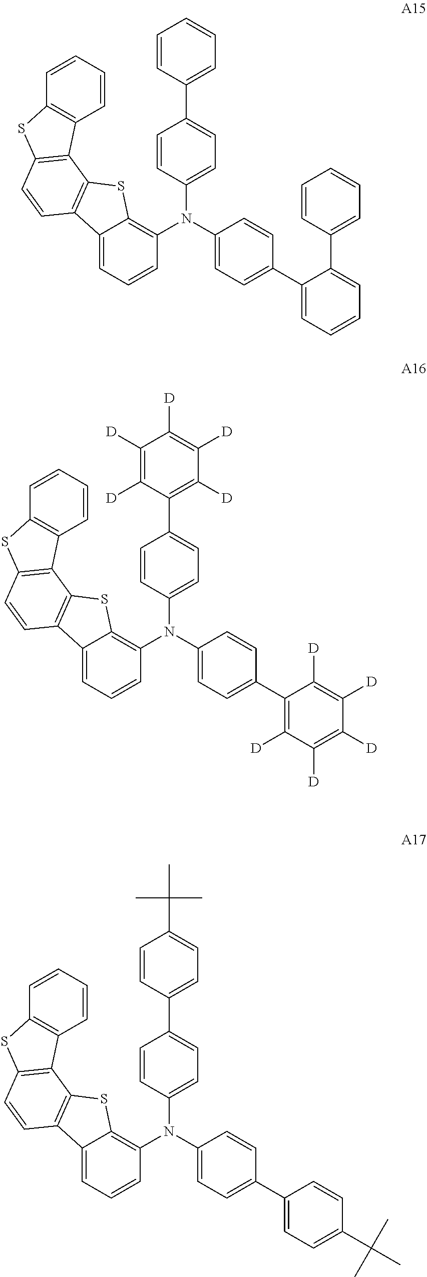 [SMILES: c1ccc(-c2ccc(N(c3ccc(-c4ccccc4-c4ccccc4)cc3)c3cccc4c3sc3c4ccc4sc5ccccc5c43)cc2)cc1 ; [2H]c1c([2H])c([2H])c(-c2ccc(N(c3ccc(-c4c([2H])c([2H])c([2H])c([2H])c4[2H])cc3)c3cccc4c3sc3c4ccc4sc5ccccc5c43)cc2)c([2H])c1[2H] ; CC(C)(C)c1ccc(-c2ccc(N(c3ccc(-c4ccc(C(C)(C)C)cc4)cc3)c3cccc4c3sc3c4ccc4sc5ccccc5c43)cc2)cc1]

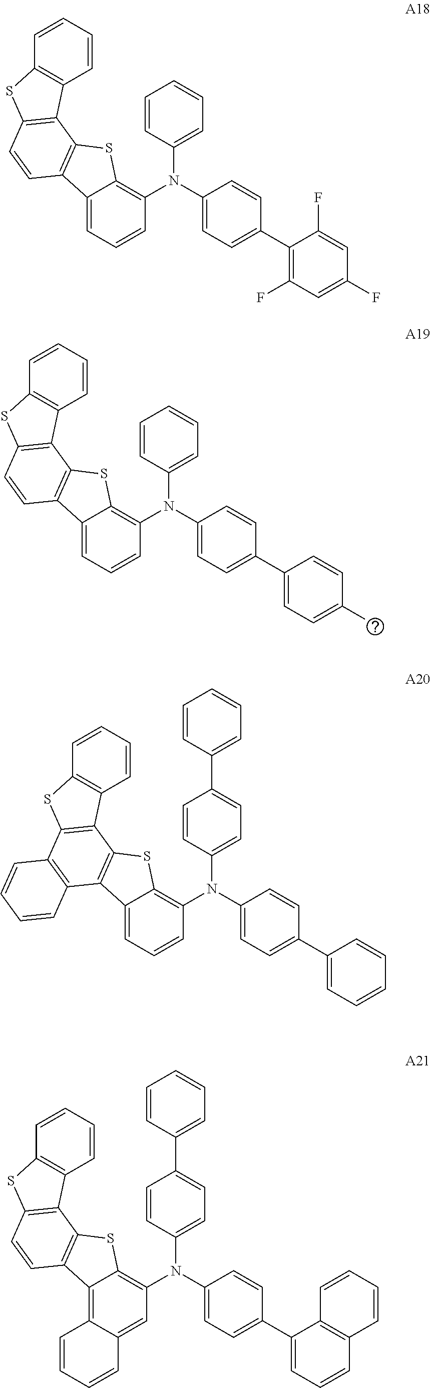 [SMILES: Fc1cc(F)c(-c2ccc(N(c3ccccc3)c3cccc4c3sc3c4ccc4sc5ccccc5c43)cc2)c(F)c1 ; Cc1ccc(-c2ccc(N(c3ccccc3)c3cccc4c3sc3c4ccc4sc5ccccc5c43)cc2)cc1 ; c1ccc(-c2ccc(N(c3ccc(-c4ccccc4)cc3)c3cccc4c3sc3c4c4ccccc4c4sc5ccccc5c43)cc2)cc1 ; c1ccc(-c2ccc(N(c3ccc(-c4cccc5ccccc45)cc3)c3cc4ccccc4c4c3sc3c4ccc4sc5ccccc5c43)cc2)cc1]

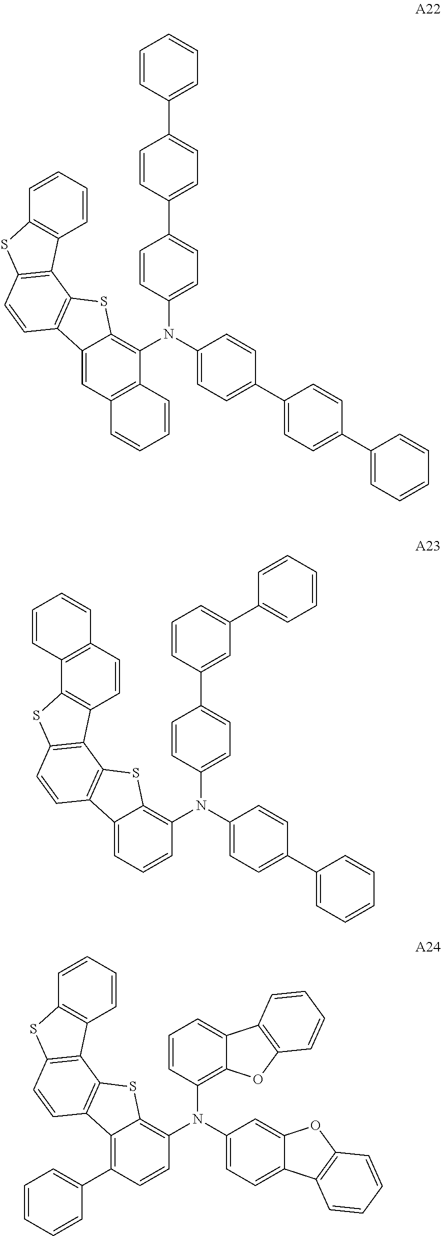 [SMILES: c1ccc(-c2ccc(-c3ccc(N(c4ccc(-c5ccc(-c6ccccc6)cc5)cc4)c4c5ccccc5cc5c4sc4c5ccc5sc6ccccc6c54)cc3)cc2)cc1 ; c1ccc(-c2ccc(N(c3ccc(-c4cccc(-c5ccccc5)c4)cc3)c3cccc4c3sc3c4ccc4sc5c6ccccc6ccc5c43)cc2)cc1 ; c1ccc(-c2ccc(N(c3ccc4c(c3)oc3ccccc34)c3cccc4c3oc3ccccc34)c3sc4c(ccc5sc6ccccc6c54)c23)cc1]

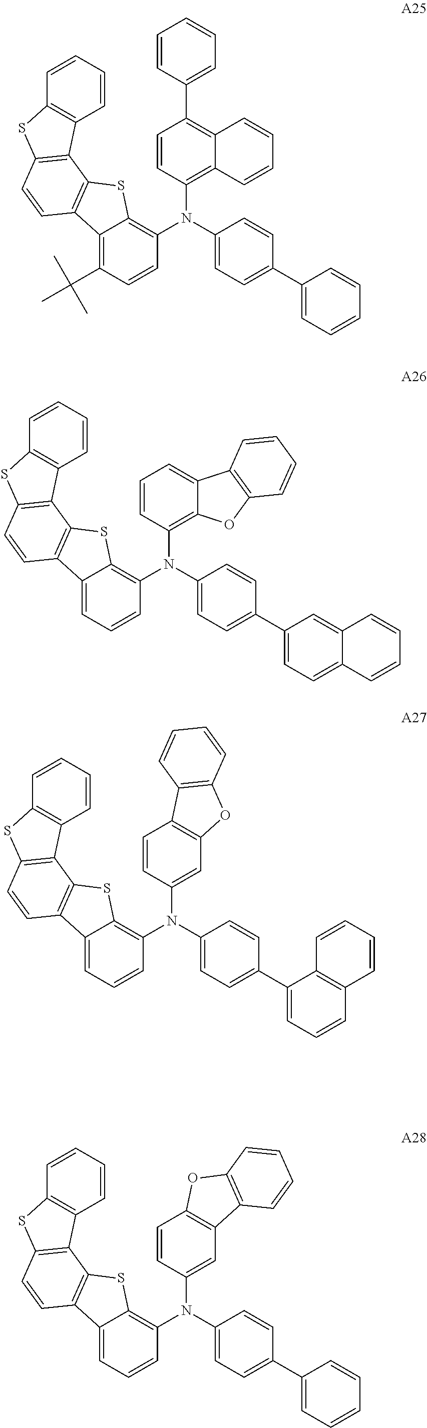 [SMILES: CC(C)(C)c1ccc(N(c2ccc(-c3ccccc3)cc2)c2ccc(-c3ccccc3)c3ccccc23)c2sc3c(ccc4sc5ccccc5c43)c12 ; c1ccc2cc(-c3ccc(N(c4cccc5c4oc4ccccc45)c4cccc5c4sc4c5ccc5sc6ccccc6c54)cc3)ccc2c1 ; c1ccc2c(-c3ccc(N(c4ccc5c(c4)oc4ccccc45)c4cccc5c4sc4c5ccc5sc6ccccc6c54)cc3)cccc2c1 ; c1ccc(-c2ccc(N(c3ccc4oc5ccccc5c4c3)c3cccc4c3sc3c4ccc4sc5ccccc5c43)cc2)cc1]

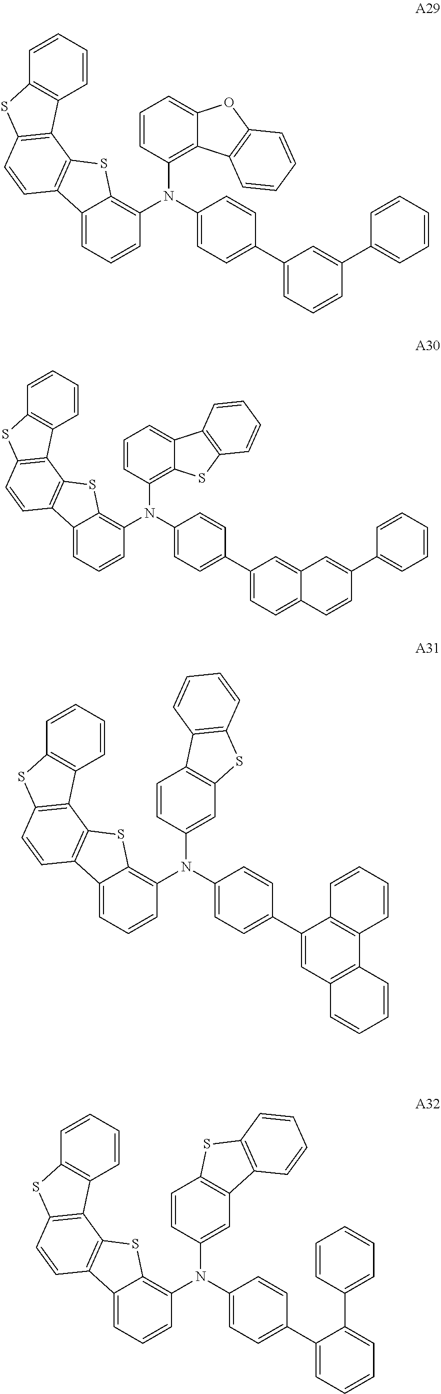 [SMILES: c1ccc(-c2cccc(-c3ccc(N(c4cccc5c4sc4c5ccc5sc6ccccc6c54)c4cccc5oc6ccccc6c45)cc3)c2)cc1 ; c1ccc(-c2ccc3ccc(-c4ccc(N(c5cccc6c5sc5ccccc56)c5cccc6c5sc5c6ccc6sc7ccccc7c65)cc4)cc3c2)cc1 ; c1ccc2c(c1)cc(-c1ccc(N(c3ccc4c(c3)sc3ccccc34)c3cccc4c3sc3c4ccc4sc5ccccc5c43)cc1)c1ccccc12 ; c1ccc(-c2ccccc2-c2ccc(N(c3ccc4sc5ccccc5c4c3)c3cccc4c3sc3c4ccc4sc5ccccc5c43)cc2)cc1]

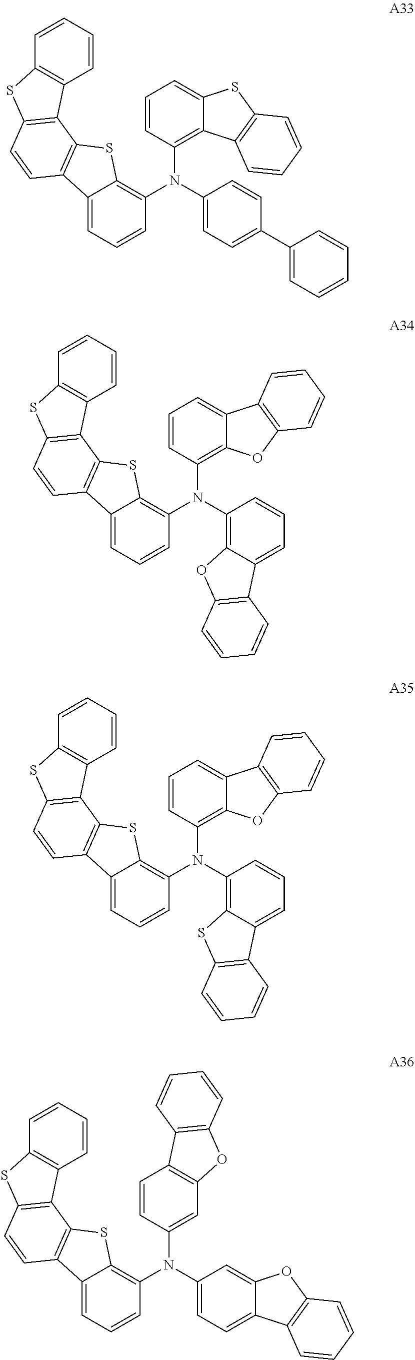 [SMILES: c1ccc(-c2ccc(N(c3cccc4c3sc3c4ccc4sc5ccccc5c43)c3cccc4sc5ccccc5c34)cc2)cc1 ; c1ccc2c(c1)oc1c(N(c3cccc4c3oc3ccccc34)c3cccc4c3sc3c4ccc4sc5ccccc5c43)cccc12 ; c1ccc2c(c1)oc1c(N(c3cccc4c3sc3ccccc34)c3cccc4c3sc3c4ccc4sc5ccccc5c43)cccc12 ; c1ccc2c(c1)oc1cc(N(c3ccc4c(c3)oc3ccccc34)c3cccc4c3sc3c4ccc4sc5ccccc5c43)ccc12]

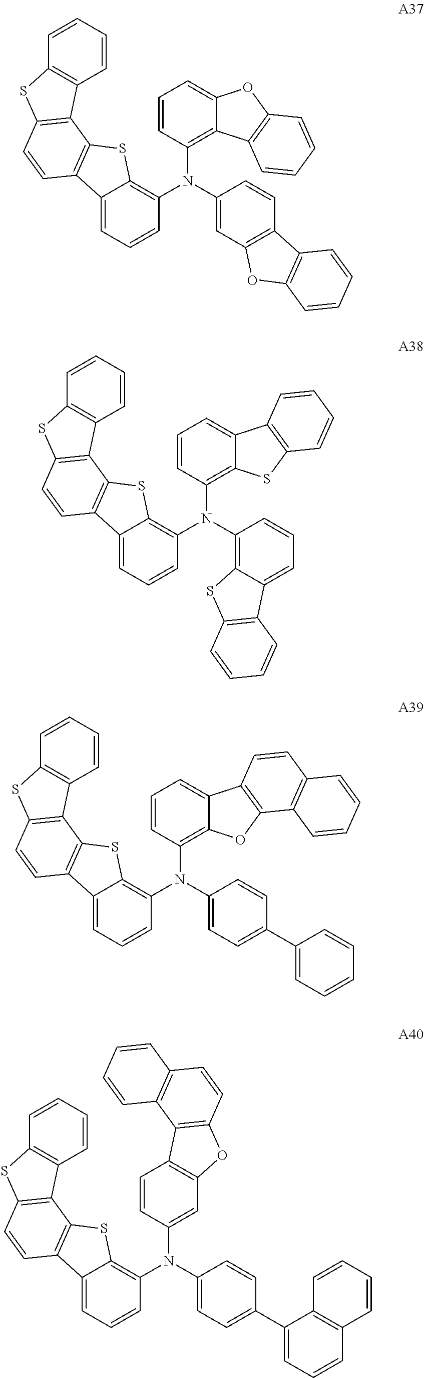 [SMILES: c1ccc2c(c1)oc1cc(N(c3cccc4c3sc3c4ccc4sc5ccccc5c43)c3cccc4oc5ccccc5c34)ccc12 ; c1ccc2c(c1)sc1c(N(c3cccc4c3sc3ccccc34)c3cccc4c3sc3c4ccc4sc5ccccc5c43)cccc12 ; c1ccc(-c2ccc(N(c3cccc4c3oc3c5ccccc5ccc43)c3cccc4c3sc3c4ccc4sc5ccccc5c43)cc2)cc1 ; c1ccc2c(-c3ccc(N(c4ccc5c(c4)oc4ccc6ccccc6c45)c4cccc5c4sc4c5ccc5sc6ccccc6c54)cc3)cccc2c1]

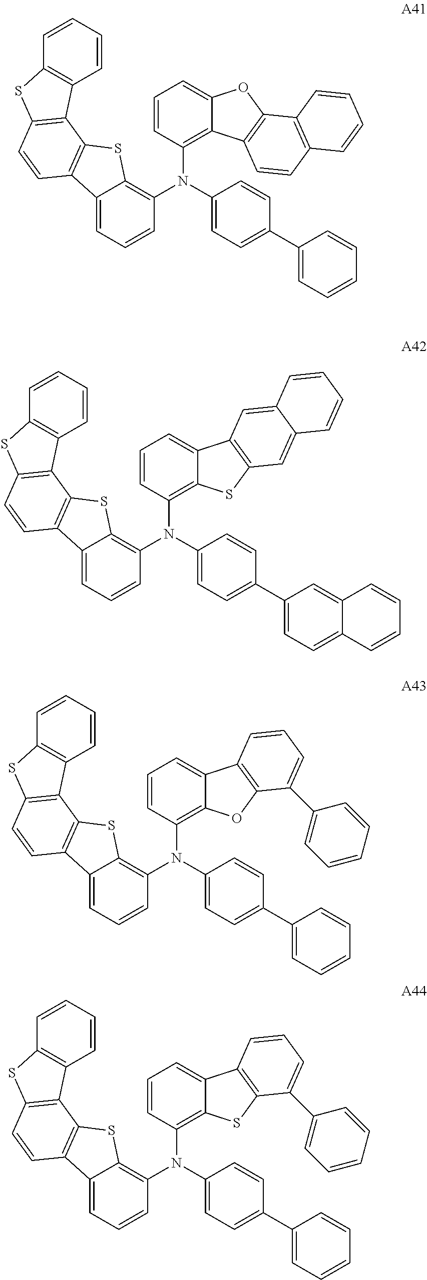 [SMILES: c1ccc(-c2ccc(N(c3cccc4c3sc3c4ccc4sc5ccccc5c43)c3cccc4oc5c6ccccc6ccc5c34)cc2)cc1 ; c1ccc2cc(-c3ccc(N(c4cccc5c4sc4cc6ccccc6cc45)c4cccc5c4sc4c5ccc5sc6ccccc6c54)cc3)ccc2c1 ; c1ccc(-c2ccc(N(c3cccc4c3oc3c(-c5ccccc5)cccc34)c3cccc4c3sc3c4ccc4sc5ccccc5c43)cc2)cc1 ; c1ccc(-c2ccc(N(c3cccc4c3sc3c(-c5ccccc5)cccc34)c3cccc4c3sc3c4ccc4sc5ccccc5c43)cc2)cc1]

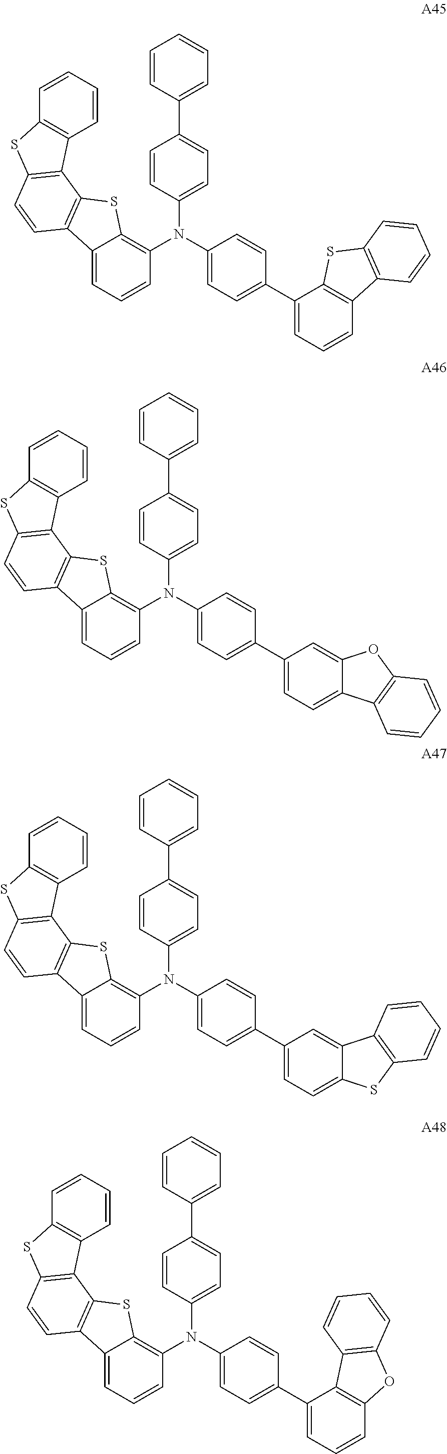 [SMILES: c1ccc(-c2ccc(N(c3ccc(-c4cccc5c4sc4ccccc45)cc3)c3cccc4c3sc3c4ccc4sc5ccccc5c43)cc2)cc1 ; c1ccc(-c2ccc(N(c3ccc(-c4ccc5c(c4)oc4ccccc45)cc3)c3cccc4c3sc3c4ccc4sc5ccccc5c43)cc2)cc1 ; c1ccc(-c2ccc(N(c3ccc(-c4ccc5sc6ccccc6c5c4)cc3)c3cccc4c3sc3c4ccc4sc5ccccc5c43)cc2)cc1 ; c1ccc(-c2ccc(N(c3ccc(-c4cccc5oc6ccccc6c45)cc3)c3cccc4c3sc3c4ccc4sc5ccccc5c43)cc2)cc1]

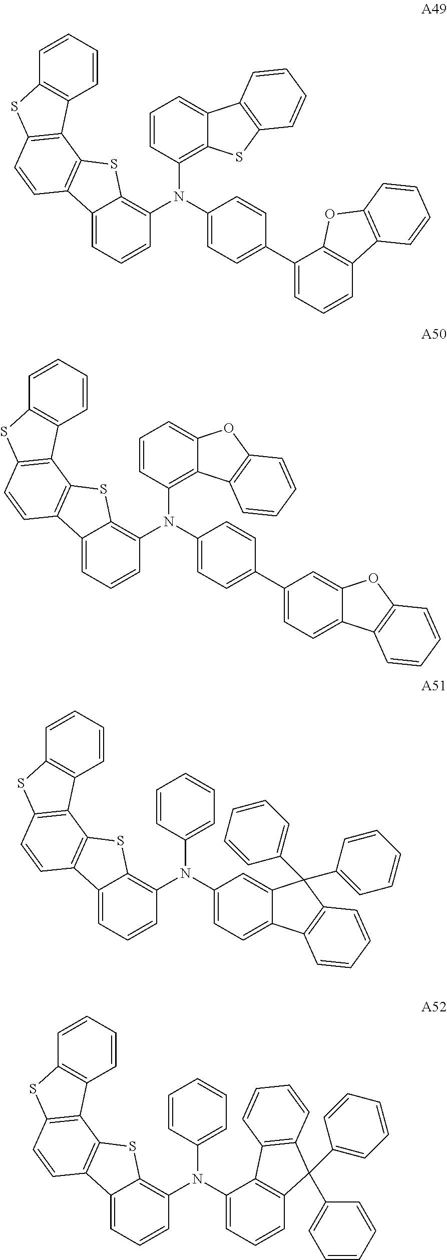 [SMILES: c1ccc2c(c1)oc1c(-c3ccc(N(c4cccc5c4sc4ccccc45)c4cccc5c4sc4c5ccc5sc6ccccc6c54)cc3)cccc12 ; c1ccc2c(c1)oc1cc(-c3ccc(N(c4cccc5c4sc4c5ccc5sc6ccccc6c54)c4cccc5oc6ccccc6c45)cc3)ccc12 ; c1ccc(N(c2ccc3c(c2)C(c2ccccc2)(c2ccccc2)c2ccccc2-3)c2cccc3c2sc2c3ccc3sc4ccccc4c32)cc1 ; c1ccc(N(c2cccc3c2-c2ccccc2C3(c2ccccc2)c2ccccc2)c2cccc3c2sc2c3ccc3sc4ccccc4c32)cc1]

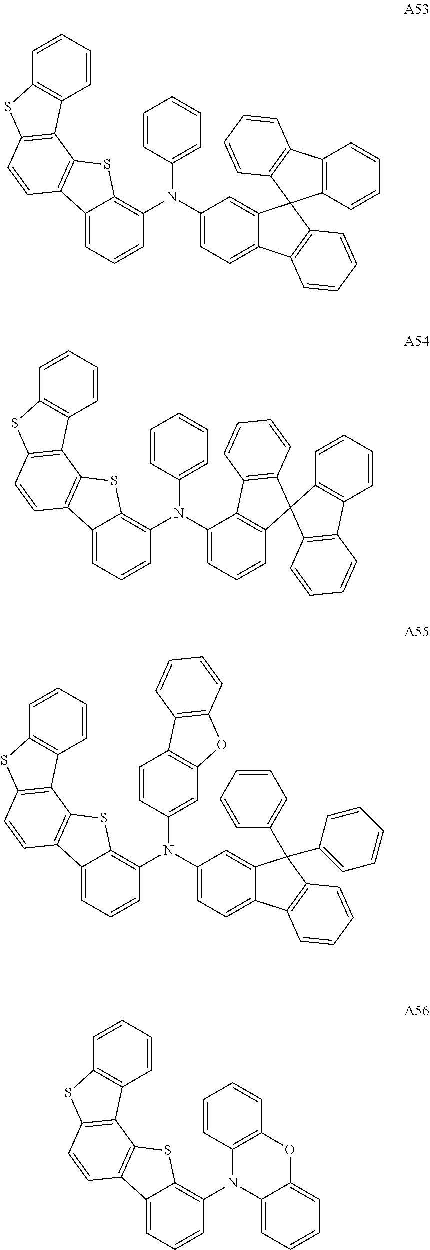 [SMILES: c1ccc(N(c2ccc3c(c2)C2(c4ccccc4-c4ccccc42)c2ccccc2-3)c2cccc3c2sc2c3ccc3sc4ccccc4c32)cc1 ; c1ccc(N(c2cccc3c2-c2ccccc2C32c3ccccc3-c3ccccc32)c2cccc3c2sc2c3ccc3sc4ccccc4c32)cc1 ; c1ccc(C2(c3ccccc3)c3ccccc3-c3ccc(N(c4ccc5c(c4)oc4ccccc45)c4cccc5c4sc4c5ccc5sc6ccccc6c54)cc32)cc1 ; c1ccc2c(c1)Oc1ccccc1N2c1cccc2c1sc1c2ccc2sc3ccccc3c21]

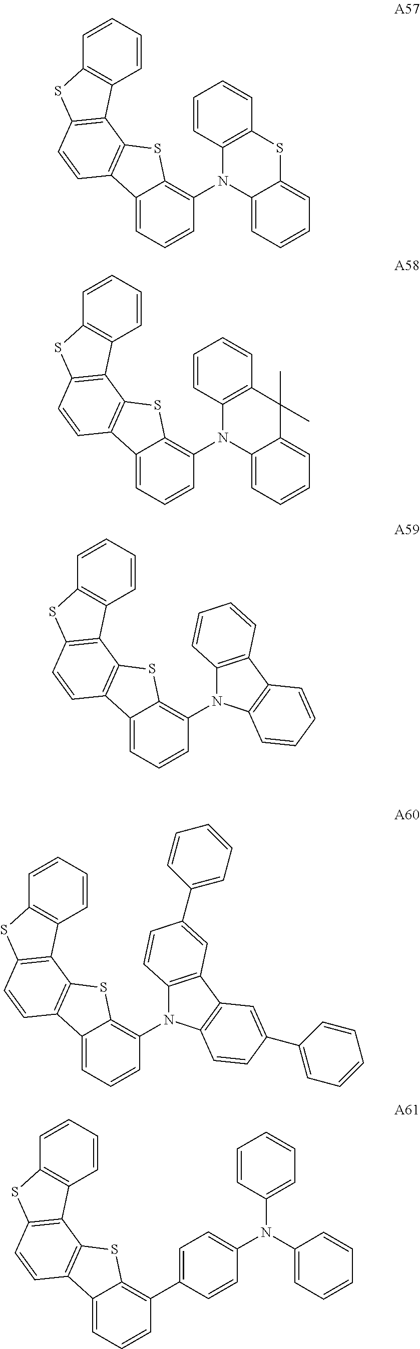 [SMILES: c1ccc2c(c1)Sc1ccccc1N2c1cccc2c1sc1c2ccc2sc3ccccc3c21 ; CC1(C)c2ccccc2N(c2cccc3c2sc2c3ccc3sc4ccccc4c32)c2ccccc21 ; c1ccc2c(c1)sc1ccc3c4cccc(-n5c6ccccc6c6ccccc65)c4sc3c12 ; c1ccc(-c2ccc3c(c2)c2cc(-c4ccccc4)ccc2n3-c2cccc3c2sc2c3ccc3sc4ccccc4c32)cc1 ; c1ccc(N(c2ccccc2)c2ccc(-c3cccc4c3sc3c4ccc4sc5ccccc5c43)cc2)cc1]

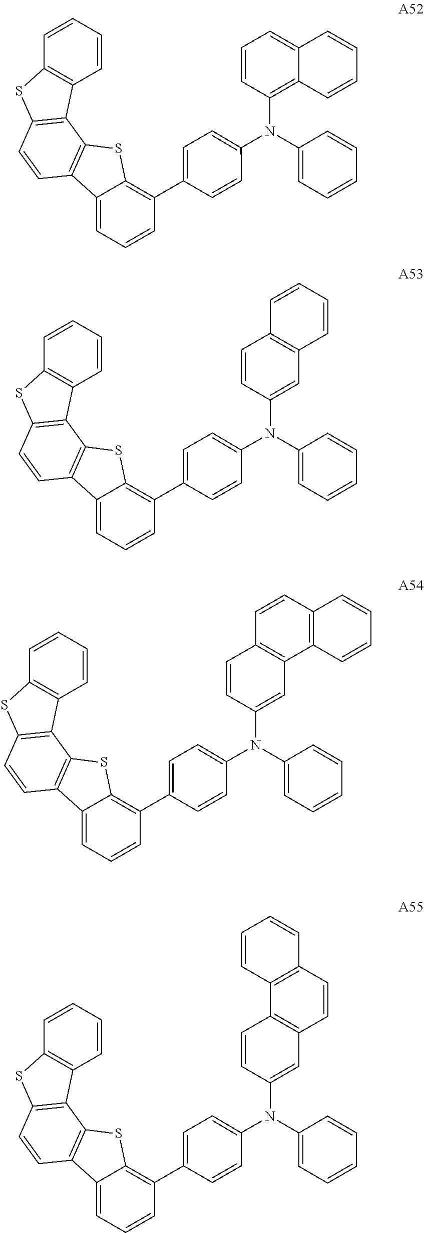 [SMILES: c1ccc(N(c2ccc(-c3cccc4c3sc3c4ccc4sc5ccccc5c43)cc2)c2cccc3ccccc23)cc1 ; c1ccc(N(c2ccc(-c3cccc4c3sc3c4ccc4sc5ccccc5c43)cc2)c2ccc3ccccc3c2)cc1 ; c1ccc(N(c2ccc(-c3cccc4c3sc3c4ccc4sc5ccccc5c43)cc2)c2ccc3ccc4ccccc4c3c2)cc1 ; c1ccc(N(c2ccc(-c3cccc4c3sc3c4ccc4sc5ccccc5c43)cc2)c2ccc3c(ccc4ccccc43)c2)cc1]

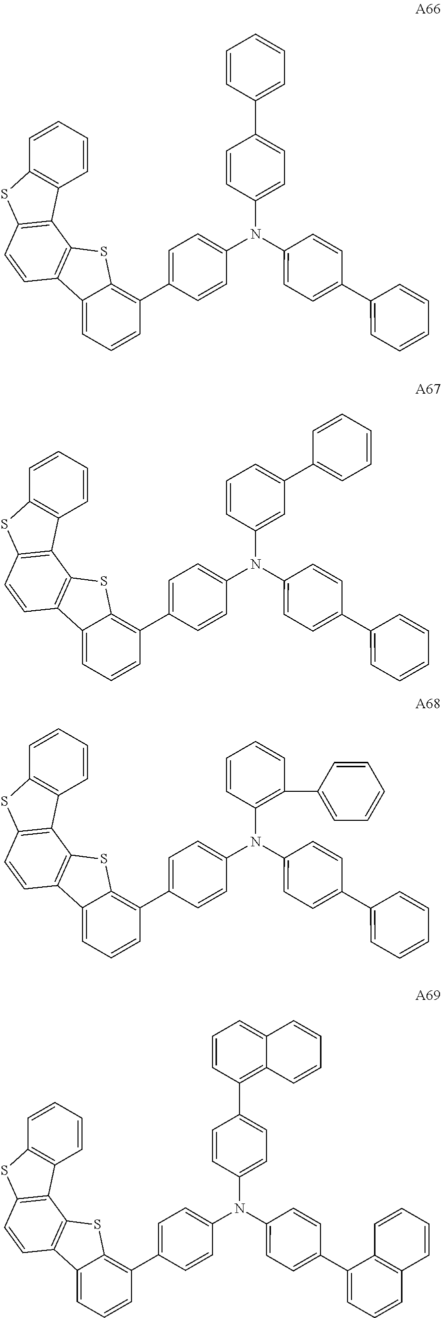 [SMILES: c1ccc(-c2ccc(N(c3ccc(-c4ccccc4)cc3)c3ccc(-c4cccc5c4sc4c5ccc5sc6ccccc6c54)cc3)cc2)cc1 ; c1ccc(-c2ccc(N(c3ccc(-c4cccc5c4sc4c5ccc5sc6ccccc6c54)cc3)c3cccc(-c4ccccc4)c3)cc2)cc1 ; c1ccc(-c2ccc(N(c3ccc(-c4cccc5c4sc4c5ccc5sc6ccccc6c54)cc3)c3ccccc3-c3ccccc3)cc2)cc1 ; c1ccc2c(-c3ccc(N(c4ccc(-c5cccc6ccccc56)cc4)c4ccc(-c5cccc6c5sc5c6ccc6sc7ccccc7c65)cc4)cc3)cccc2c1]

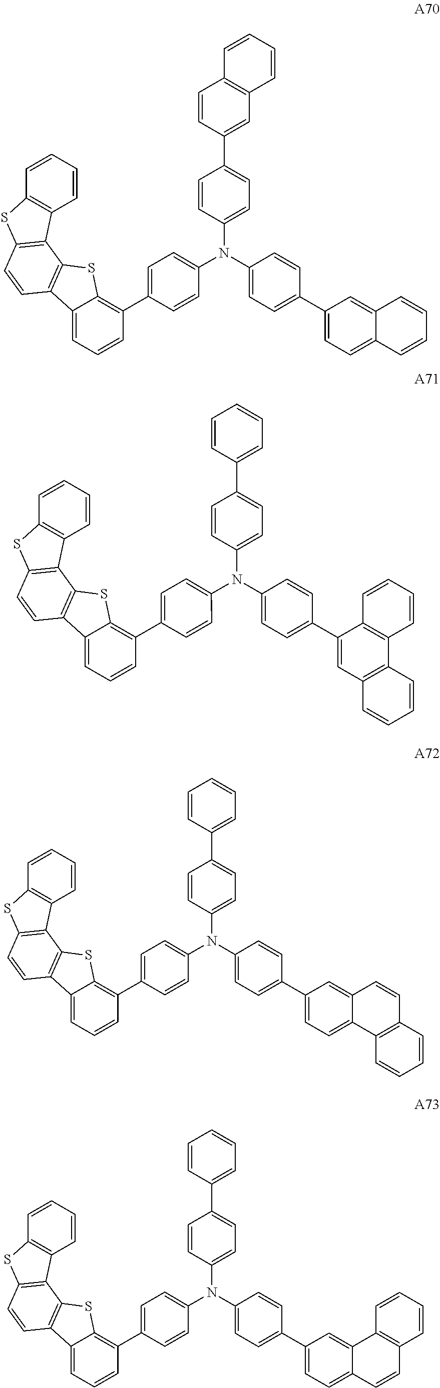 [SMILES: c1ccc2cc(-c3ccc(N(c4ccc(-c5ccc6ccccc6c5)cc4)c4ccc(-c5cccc6c5sc5c6ccc6sc7ccccc7c65)cc4)cc3)ccc2c1 ; c1ccc(-c2ccc(N(c3ccc(-c4cc5ccccc5c5ccccc45)cc3)c3ccc(-c4cccc5c4sc4c5ccc5sc6ccccc6c54)cc3)cc2)cc1 ; c1ccc(-c2ccc(N(c3ccc(-c4ccc5c(ccc6ccccc65)c4)cc3)c3ccc(-c4cccc5c4sc4c5ccc5sc6ccccc6c54)cc3)cc2)cc1 ; c1ccc(-c2ccc(N(c3ccc(-c4ccc5ccc6ccccc6c5c4)cc3)c3ccc(-c4cccc5c4sc4c5ccc5sc6ccccc6c54)cc3)cc2)cc1]

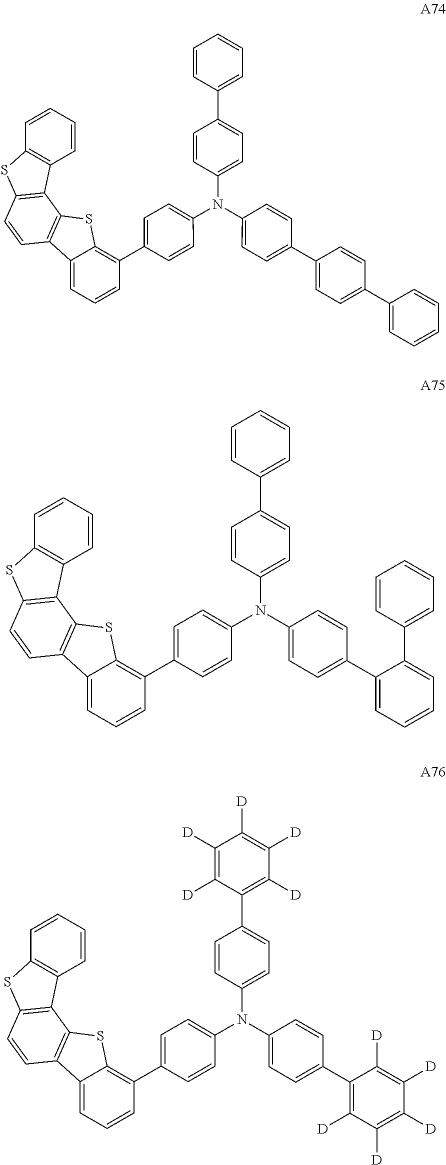 [SMILES: c1ccc(-c2ccc(-c3ccc(N(c4ccc(-c5ccccc5)cc4)c4ccc(-c5cccc6c5sc5c6ccc6sc7ccccc7c65)cc4)cc3)cc2)cc1 ; c1ccc(-c2ccc(N(c3ccc(-c4ccccc4-c4ccccc4)cc3)c3ccc(-c4cccc5c4sc4c5ccc5sc6ccccc6c54)cc3)cc2)cc1 ; [2H]c1c([2H])c([2H])c(-c2ccc(N(c3ccc(-c4c([2H])c([2H])c([2H])c([2H])c4[2H])cc3)c3ccc(-c4cccc5c4sc4c5ccc5sc6ccccc6c54)cc3)cc2)c([2H])c1[2H]]

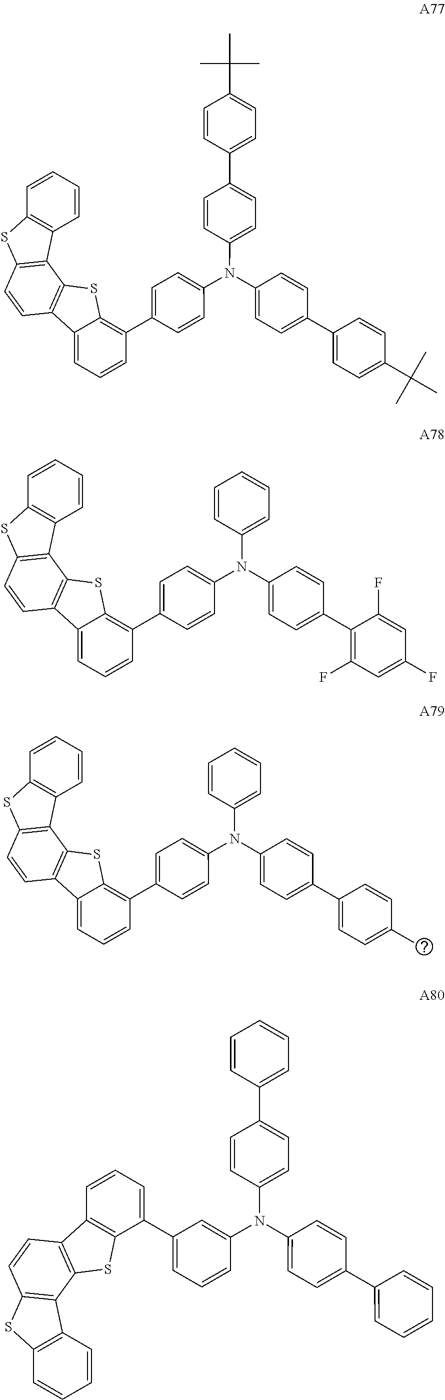 [SMILES: CC(C)(C)c1ccc(-c2ccc(N(c3ccc(-c4ccc(C(C)(C)C)cc4)cc3)c3ccc(-c4cccc5c4sc4c5ccc5sc6ccccc6c54)cc3)cc2)cc1 ; Fc1cc(F)c(-c2ccc(N(c3ccccc3)c3ccc(-c4cccc5c4sc4c5ccc5sc6ccccc6c54)cc3)cc2)c(F)c1 ; Cc1ccc(-c2ccc(N(c3ccccc3)c3ccc(-c4cccc5c4sc4c5ccc5sc6ccccc6c54)cc3)cc2)cc1 ; c1ccc(-c2ccc(N(c3ccc(-c4ccccc4)cc3)c3cccc(-c4cccc5c4sc4c5ccc5sc6ccccc6c54)c3)cc2)cc1]

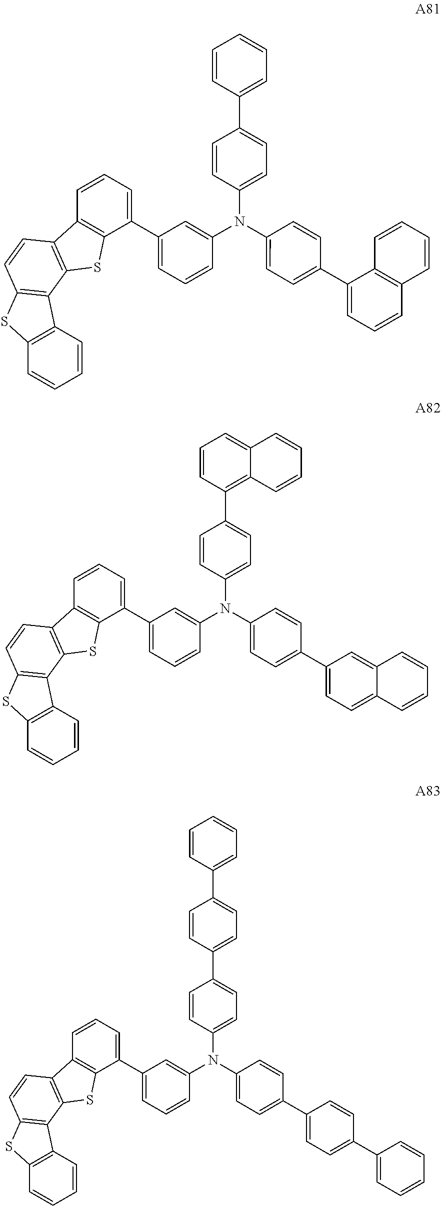 [SMILES: c1ccc(-c2ccc(N(c3ccc(-c4cccc5ccccc45)cc3)c3cccc(-c4cccc5c4sc4c5ccc5sc6ccccc6c54)c3)cc2)cc1 ; c1cc(-c2cccc3c2sc2c3ccc3sc4ccccc4c32)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6c5sc5c6ccc6sc7ccccc7c65)c4)cc3)cc2)cc1]

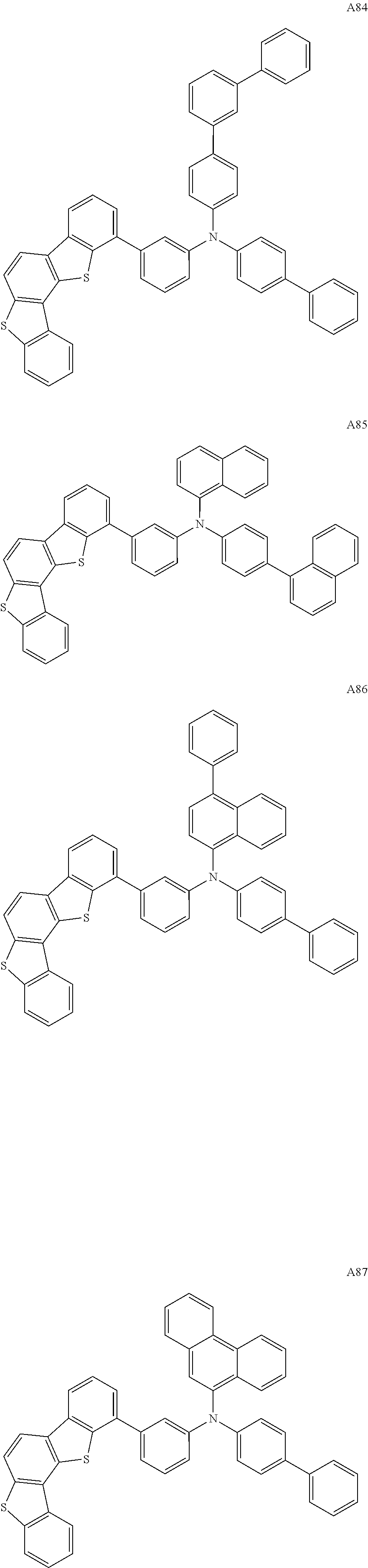 [SMILES: c1ccc(-c2ccc(N(c3ccc(-c4cccc(-c5ccccc5)c4)cc3)c3cccc(-c4cccc5c4sc4c5ccc5sc6ccccc6c54)c3)cc2)cc1 ; c1cc(-c2cccc3c2sc2c3ccc3sc4ccccc4c32)cc(N(c2ccc(-c3cccc4ccccc34)cc2)c2cccc3ccccc23)c1 ; c1ccc(-c2ccc(N(c3cccc(-c4cccc5c4sc4c5ccc5sc6ccccc6c54)c3)c3ccc(-c4ccccc4)c4ccccc34)cc2)cc1 ; c1ccc(-c2ccc(N(c3cccc(-c4cccc5c4sc4c5ccc5sc6ccccc6c54)c3)c3cc4ccccc4c4ccccc34)cc2)cc1]

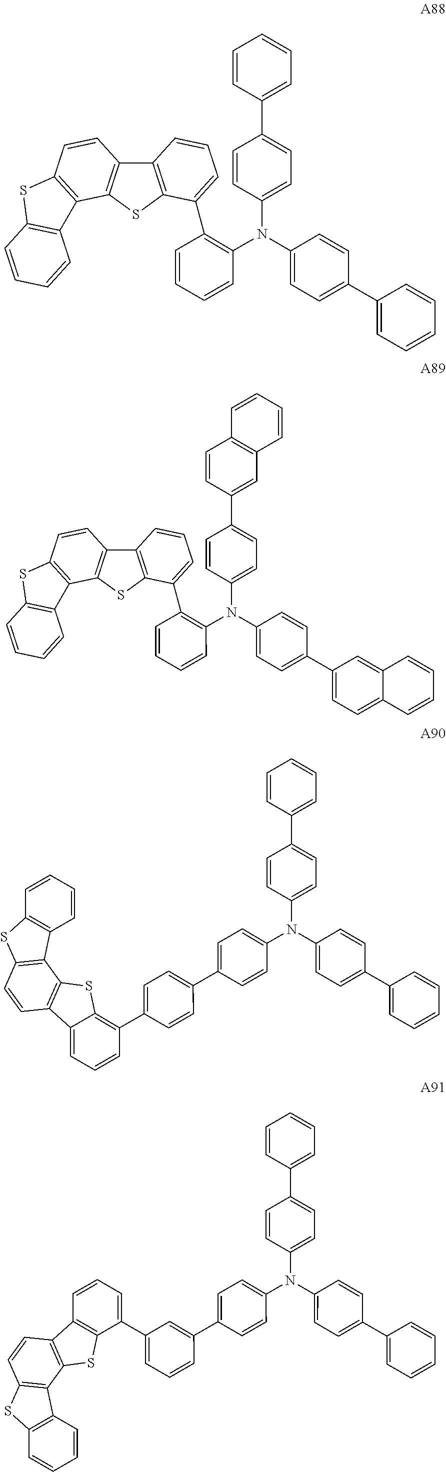 [SMILES: c1ccc(-c2ccc(N(c3ccc(-c4ccccc4)cc3)c3ccccc3-c3cccc4c3sc3c4ccc4sc5ccccc5c43)cc2)cc1 ; c1ccc(N(c2ccc(-c3ccc4ccccc4c3)cc2)c2ccc(-c3ccc4ccccc4c3)cc2)c(-c2cccc3c2sc2c3ccc3sc4ccccc4c32)c1 ; c1ccc(-c2ccc(N(c3ccc(-c4ccccc4)cc3)c3ccc(-c4ccc(-c5cccc6c5sc5c6ccc6sc7ccccc7c65)cc4)cc3)cc2)cc1 ; c1ccc(-c2ccc(N(c3ccc(-c4ccccc4)cc3)c3ccc(-c4cccc(-c5cccc6c5sc5c6ccc6sc7ccccc7c65)c4)cc3)cc2)cc1]

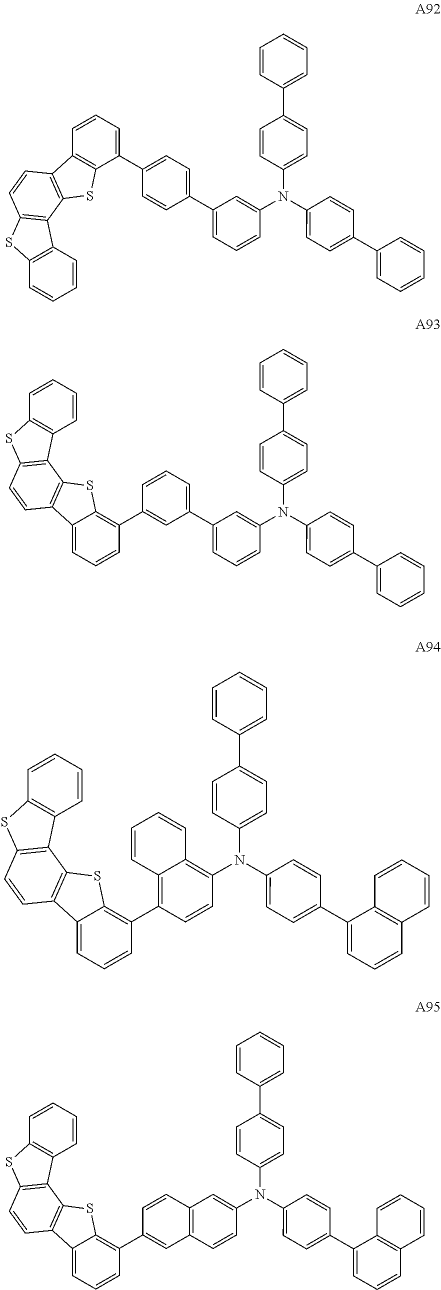 [SMILES: c1ccc(-c2ccc(N(c3ccc(-c4ccccc4)cc3)c3cccc(-c4ccc(-c5cccc6c5sc5c6ccc6sc7ccccc7c65)cc4)c3)cc2)cc1 ; c1ccc(-c2ccc(N(c3ccc(-c4ccccc4)cc3)c3cccc(-c4cccc(-c5cccc6c5sc5c6ccc6sc7ccccc7c65)c4)c3)cc2)cc1 ; c1ccc(-c2ccc(N(c3ccc(-c4cccc5ccccc45)cc3)c3ccc(-c4cccc5c4sc4c5ccc5sc6ccccc6c54)c4ccccc34)cc2)cc1 ; c1ccc(-c2ccc(N(c3ccc(-c4cccc5ccccc45)cc3)c3ccc4cc(-c5cccc6c5sc5c6ccc6sc7ccccc7c65)ccc4c3)cc2)cc1]

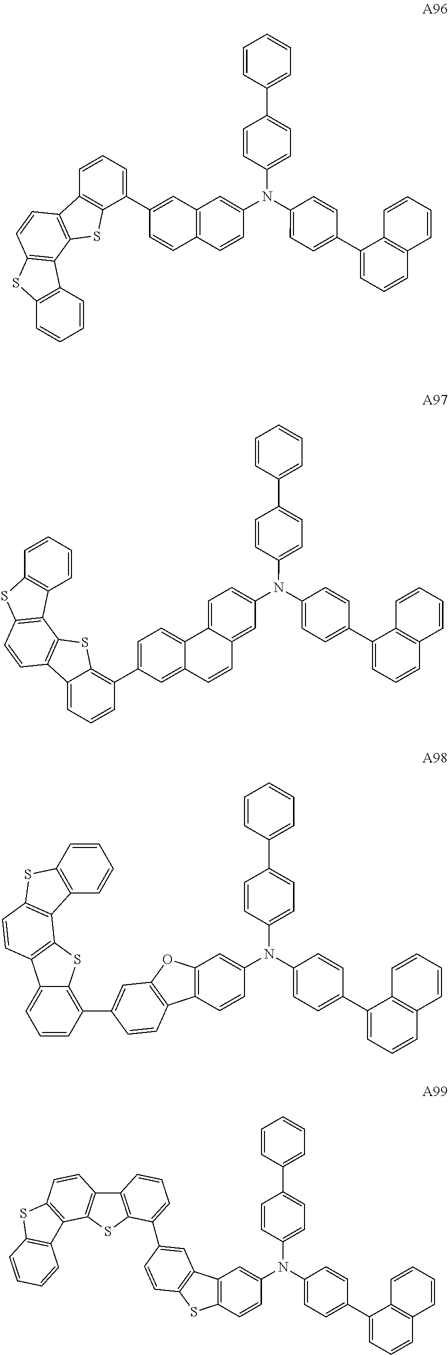 [SMILES: c1ccc(-c2ccc(N(c3ccc(-c4cccc5ccccc45)cc3)c3ccc4ccc(-c5cccc6c5sc5c6ccc6sc7ccccc7c65)cc4c3)cc2)cc1 ; c1ccc(-c2ccc(N(c3ccc(-c4cccc5ccccc45)cc3)c3ccc4c(ccc5cc(-c6cccc7c6sc6c7ccc7sc8ccccc8c76)ccc54)c3)cc2)cc1 ; c1ccc(-c2ccc(N(c3ccc(-c4cccc5ccccc45)cc3)c3ccc4c(c3)oc3cc(-c5cccc6c5sc5c6ccc6sc7ccccc7c65)ccc34)cc2)cc1 ; c1ccc(-c2ccc(N(c3ccc(-c4cccc5ccccc45)cc3)c3ccc4sc5ccc(-c6cccc7c6sc6c7ccc7sc8ccccc8c76)cc5c4c3)cc2)cc1]

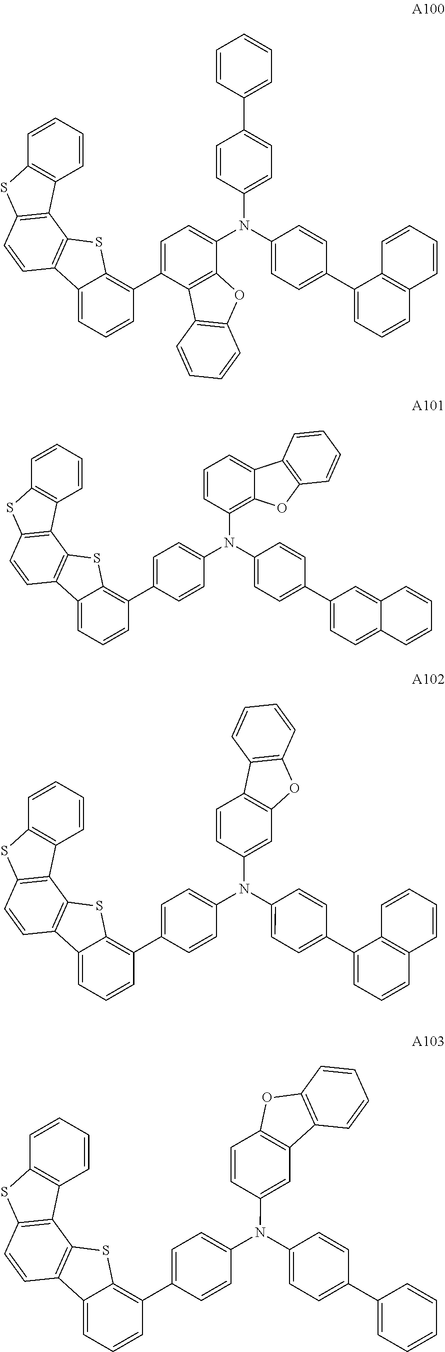 [SMILES: c1ccc(-c2ccc(N(c3ccc(-c4cccc5ccccc45)cc3)c3ccc(-c4cccc5c4sc4c5ccc5sc6ccccc6c54)c4c3oc3ccccc34)cc2)cc1 ; c1ccc2cc(-c3ccc(N(c4ccc(-c5cccc6c5sc5c6ccc6sc7ccccc7c65)cc4)c4cccc5c4oc4ccccc45)cc3)ccc2c1 ; c1ccc2c(-c3ccc(N(c4ccc(-c5cccc6c5sc5c6ccc6sc7ccccc7c65)cc4)c4ccc5c(c4)oc4ccccc45)cc3)cccc2c1 ; c1ccc(-c2ccc(N(c3ccc(-c4cccc5c4sc4c5ccc5sc6ccccc6c54)cc3)c3ccc4oc5ccccc5c4c3)cc2)cc1]

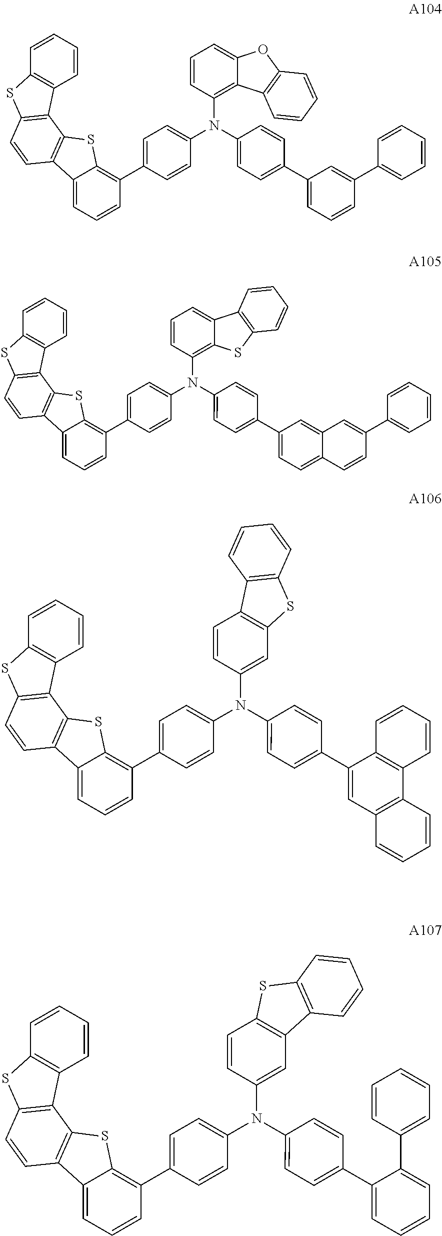 [SMILES: c1ccc(-c2cccc(-c3ccc(N(c4ccc(-c5cccc6c5sc5c6ccc6sc7ccccc7c65)cc4)c4cccc5oc6ccccc6c45)cc3)c2)cc1 ; c1ccc(-c2ccc3ccc(-c4ccc(N(c5ccc(-c6cccc7c6sc6c7ccc7sc8ccccc8c76)cc5)c5cccc6c5sc5ccccc56)cc4)cc3c2)cc1 ; c1ccc2c(c1)cc(-c1ccc(N(c3ccc(-c4cccc5c4sc4c5ccc5sc6ccccc6c54)cc3)c3ccc4c(c3)sc3ccccc34)cc1)c1ccccc12 ; c1ccc(-c2ccccc2-c2ccc(N(c3ccc(-c4cccc5c4sc4c5ccc5sc6ccccc6c54)cc3)c3ccc4sc5ccccc5c4c3)cc2)cc1]

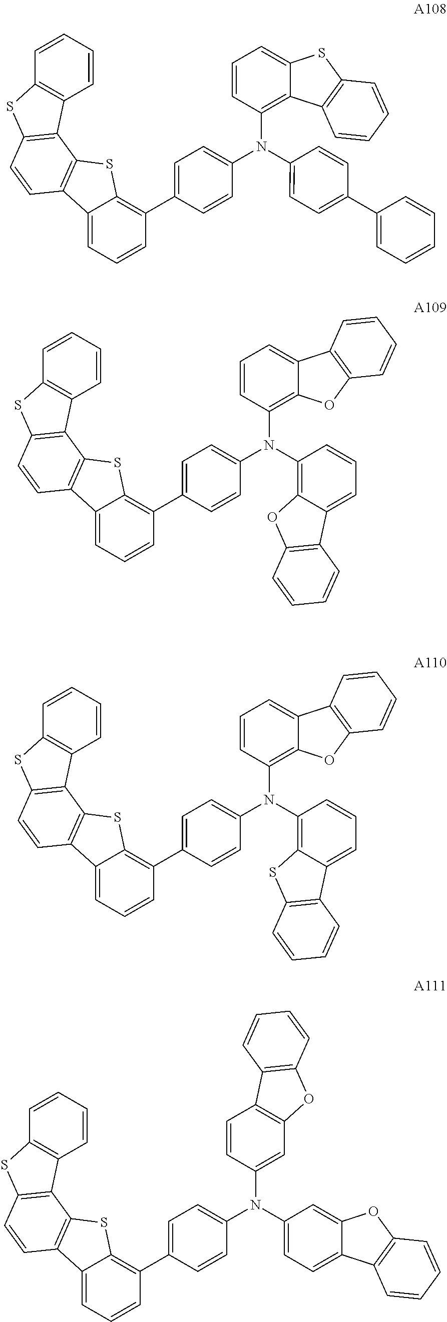 [SMILES: c1ccc(-c2ccc(N(c3ccc(-c4cccc5c4sc4c5ccc5sc6ccccc6c54)cc3)c3cccc4sc5ccccc5c34)cc2)cc1 ; c1ccc2c(c1)oc1c(N(c3ccc(-c4cccc5c4sc4c5ccc5sc6ccccc6c54)cc3)c3cccc4c3oc3ccccc34)cccc12 ; c1ccc2c(c1)oc1c(N(c3ccc(-c4cccc5c4sc4c5ccc5sc6ccccc6c54)cc3)c3cccc4c3sc3ccccc34)cccc12 ; c1ccc2c(c1)oc1cc(N(c3ccc(-c4cccc5c4sc4c5ccc5sc6ccccc6c54)cc3)c3ccc4c(c3)oc3ccccc34)ccc12]

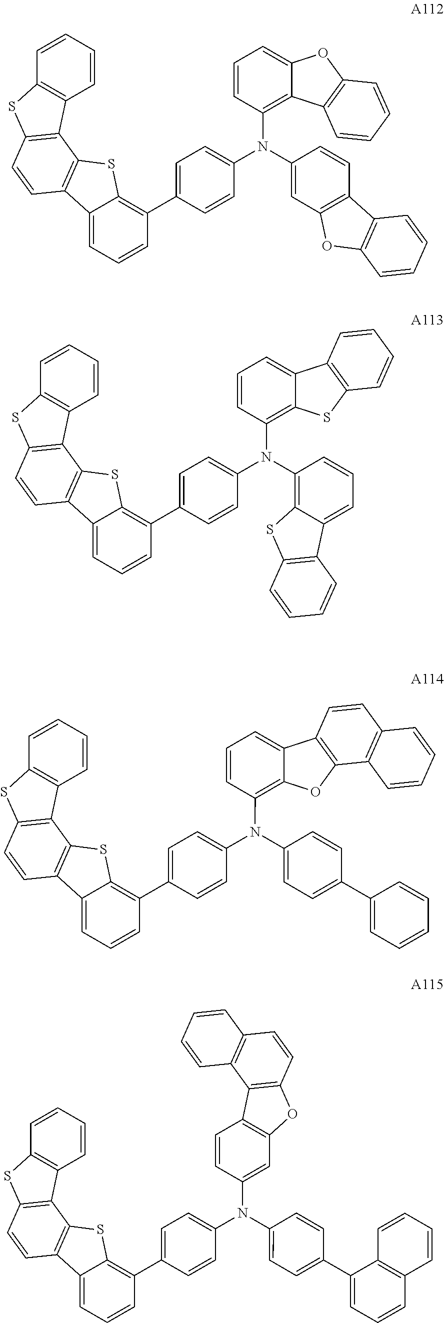 [SMILES: c1ccc2c(c1)oc1cc(N(c3ccc(-c4cccc5c4sc4c5ccc5sc6ccccc6c54)cc3)c3cccc4oc5ccccc5c34)ccc12 ; c1ccc2c(c1)sc1c(N(c3ccc(-c4cccc5c4sc4c5ccc5sc6ccccc6c54)cc3)c3cccc4c3sc3ccccc34)cccc12 ; c1ccc(-c2ccc(N(c3ccc(-c4cccc5c4sc4c5ccc5sc6ccccc6c54)cc3)c3cccc4c3oc3c5ccccc5ccc43)cc2)cc1 ; c1ccc2c(-c3ccc(N(c4ccc(-c5cccc6c5sc5c6ccc6sc7ccccc7c65)cc4)c4ccc5c(c4)oc4ccc6ccccc6c45)cc3)cccc2c1]

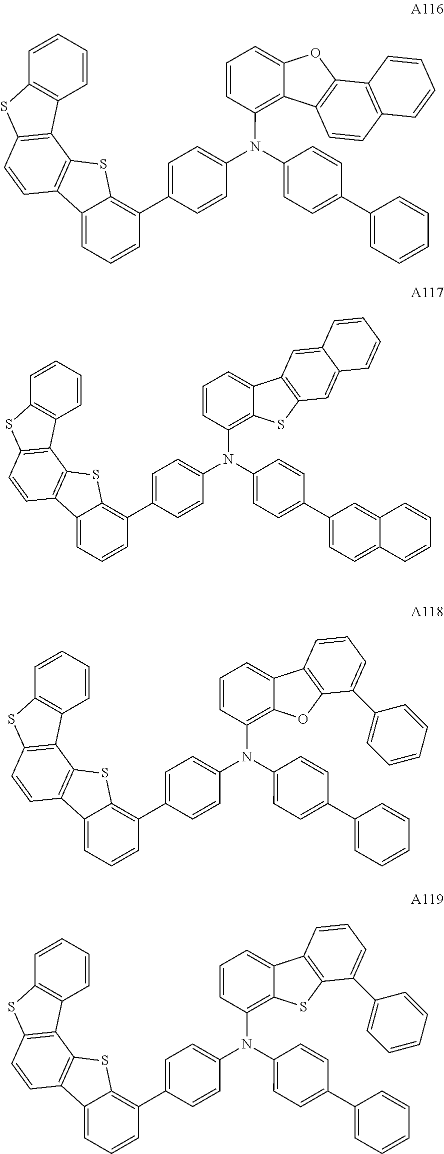 [SMILES: c1ccc(-c2ccc(N(c3ccc(-c4cccc5c4sc4c5ccc5sc6ccccc6c54)cc3)c3cccc4oc5c6ccccc6ccc5c34)cc2)cc1 ; c1ccc2cc(-c3ccc(N(c4ccc(-c5cccc6c5sc5c6ccc6sc7ccccc7c65)cc4)c4cccc5c4sc4cc6ccccc6cc45)cc3)ccc2c1 ; c1ccc(-c2ccc(N(c3ccc(-c4cccc5c4sc4c5ccc5sc6ccccc6c54)cc3)c3cccc4c3oc3c(-c5ccccc5)cccc34)cc2)cc1 ; c1ccc(-c2ccc(N(c3ccc(-c4cccc5c4sc4c5ccc5sc6ccccc6c54)cc3)c3cccc4c3sc3c(-c5ccccc5)cccc34)cc2)cc1]

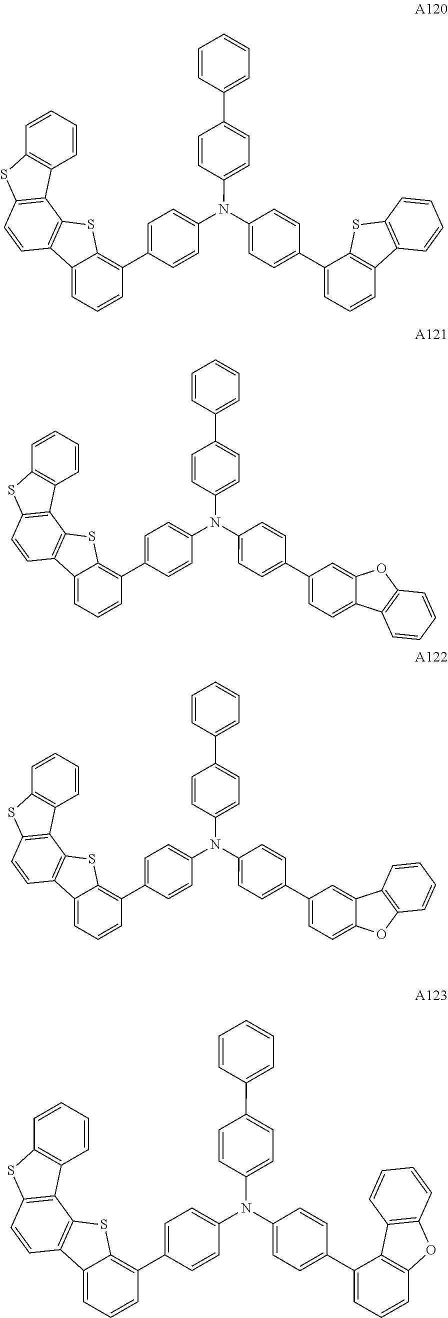 [SMILES: c1ccc(-c2ccc(N(c3ccc(-c4cccc5c4sc4ccccc45)cc3)c3ccc(-c4cccc5c4sc4c5ccc5sc6ccccc6c54)cc3)cc2)cc1 ; c1ccc(-c2ccc(N(c3ccc(-c4ccc5c(c4)oc4ccccc45)cc3)c3ccc(-c4cccc5c4sc4c5ccc5sc6ccccc6c54)cc3)cc2)cc1 ; c1ccc(-c2ccc(N(c3ccc(-c4ccc5oc6ccccc6c5c4)cc3)c3ccc(-c4cccc5c4sc4c5ccc5sc6ccccc6c54)cc3)cc2)cc1 ; c1ccc(-c2ccc(N(c3ccc(-c4cccc5c4sc4c5ccc5sc6ccccc6c54)cc3)c3ccc(-c4cccc5oc6ccccc6c45)cc3)cc2)cc1]

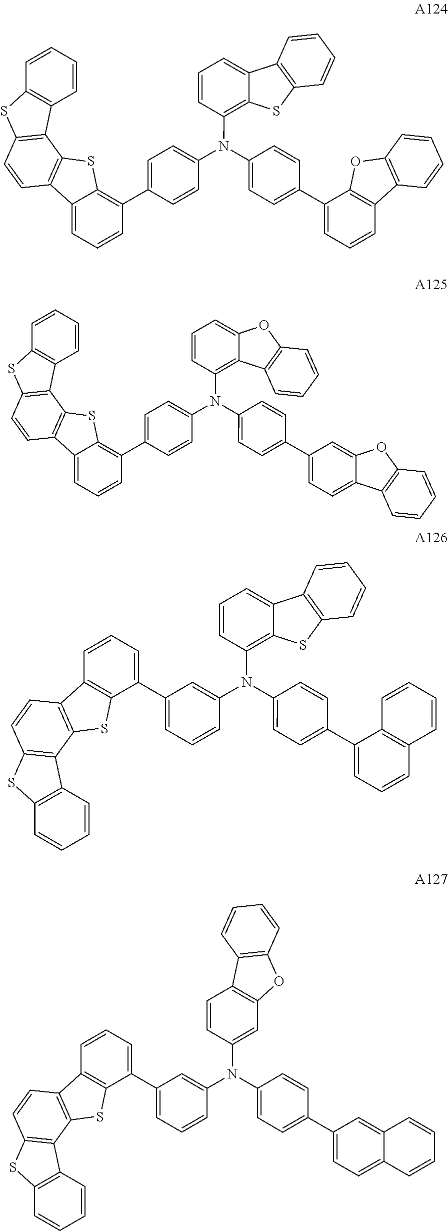 [SMILES: c1ccc2c(c1)oc1c(-c3ccc(N(c4ccc(-c5cccc6c5sc5c6ccc6sc7ccccc7c65)cc4)c4cccc5c4sc4ccccc45)cc3)cccc12 ; c1ccc2c(c1)oc1cc(-c3ccc(N(c4ccc(-c5cccc6c5sc5c6ccc6sc7ccccc7c65)cc4)c4cccc5oc6ccccc6c45)cc3)ccc12 ; c1cc(-c2cccc3c2sc2c3ccc3sc4ccccc4c32)cc(N(c2ccc(-c3cccc4ccccc34)cc2)c2cccc3c2sc2ccccc23)c1 ; c1cc(-c2cccc3c2sc2c3ccc3sc4ccccc4c32)cc(N(c2ccc(-c3ccc4ccccc4c3)cc2)c2ccc3c(c2)oc2ccccc23)c1]

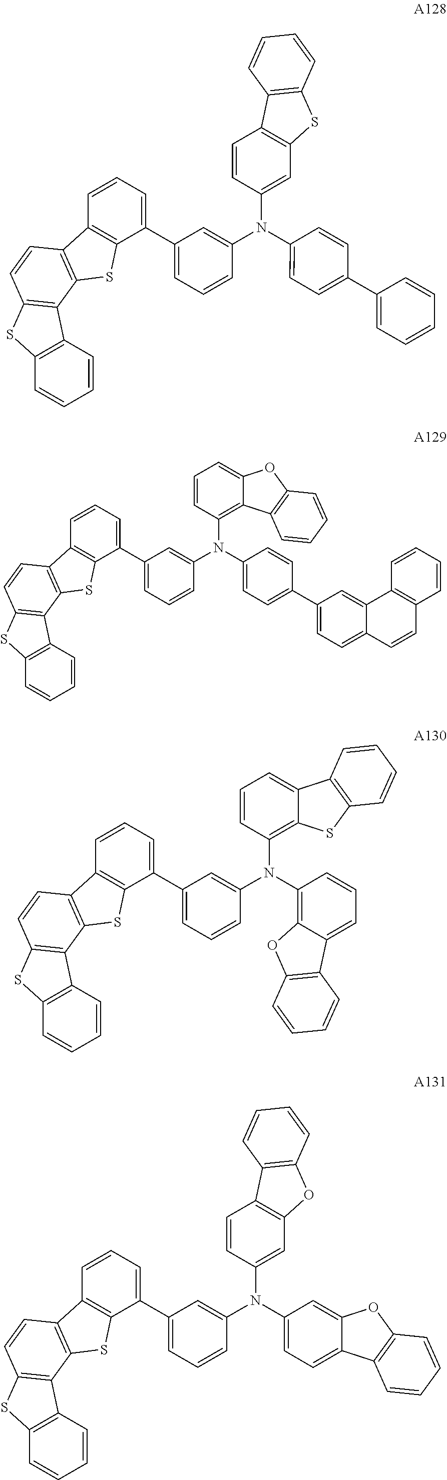 [SMILES: c1ccc(-c2ccc(N(c3cccc(-c4cccc5c4sc4c5ccc5sc6ccccc6c54)c3)c3ccc4c(c3)sc3ccccc34)cc2)cc1 ; c1cc(-c2cccc3c2sc2c3ccc3sc4ccccc4c32)cc(N(c2ccc(-c3ccc4ccc5ccccc5c4c3)cc2)c2cccc3oc4ccccc4c23)c1 ; c1cc(-c2cccc3c2sc2c3ccc3sc4ccccc4c32)cc(N(c2cccc3c2oc2ccccc23)c2cccc3c2sc2ccccc23)c1 ; c1cc(-c2cccc3c2sc2c3ccc3sc4ccccc4c32)cc(N(c2ccc3c(c2)oc2ccccc23)c2ccc3c(c2)oc2ccccc23)c1]

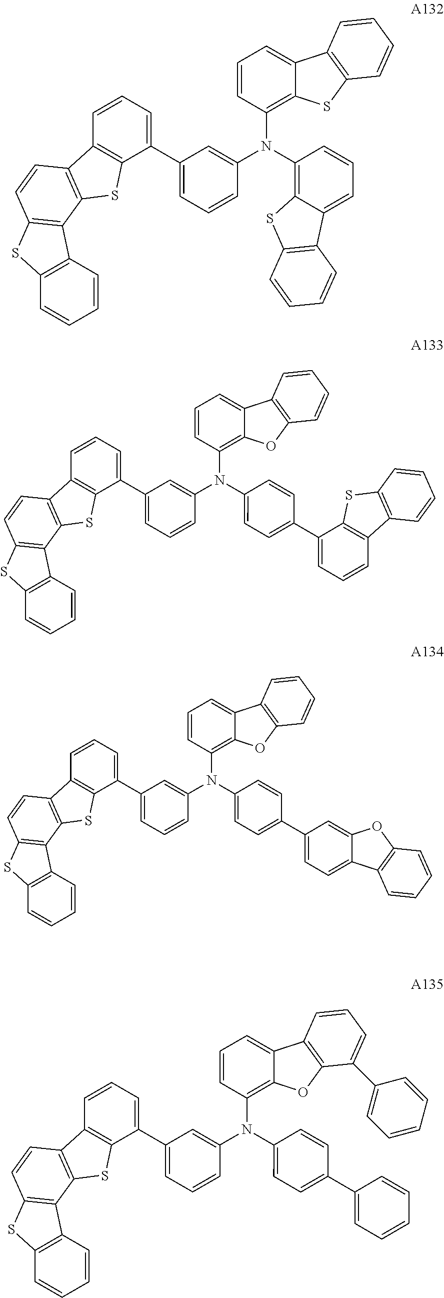 [SMILES: c1cc(-c2cccc3c2sc2c3ccc3sc4ccccc4c32)cc(N(c2cccc3c2sc2ccccc23)c2cccc3c2sc2ccccc23)c1 ; c1cc(-c2cccc3c2sc2c3ccc3sc4ccccc4c32)cc(N(c2ccc(-c3cccc4c3sc3ccccc34)cc2)c2cccc3c2oc2ccccc23)c1 ; c1cc(-c2cccc3c2sc2c3ccc3sc4ccccc4c32)cc(N(c2ccc(-c3ccc4c(c3)oc3ccccc34)cc2)c2cccc3c2oc2ccccc23)c1 ; c1ccc(-c2ccc(N(c3cccc(-c4cccc5c4sc4c5ccc5sc6ccccc6c54)c3)c3cccc4c3oc3c(-c5ccccc5)cccc34)cc2)cc1]

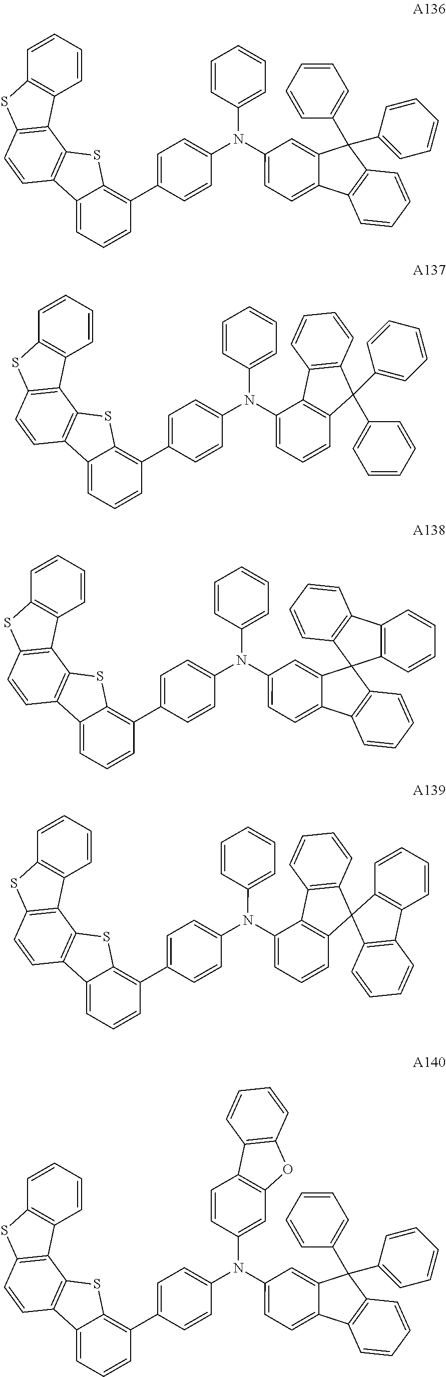 [SMILES: c1ccc(N(c2ccc(-c3cccc4c3sc3c4ccc4sc5ccccc5c43)cc2)c2ccc3c(c2)C(c2ccccc2)(c2ccccc2)c2ccccc2-3)cc1 ; c1ccc(N(c2ccc(-c3cccc4c3sc3c4ccc4sc5ccccc5c43)cc2)c2cccc3c2-c2ccccc2C3(c2ccccc2)c2ccccc2)cc1 ; c1ccc(N(c2ccc(-c3cccc4c3sc3c4ccc4sc5ccccc5c43)cc2)c2ccc3c(c2)C2(c4ccccc4-c4ccccc42)c2ccccc2-3)cc1 ; c1ccc(N(c2ccc(-c3cccc4c3sc3c4ccc4sc5ccccc5c43)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6c5sc5c6ccc6sc7ccccc7c65)cc4)c4ccc5c(c4)oc4ccccc45)cc32)cc1]

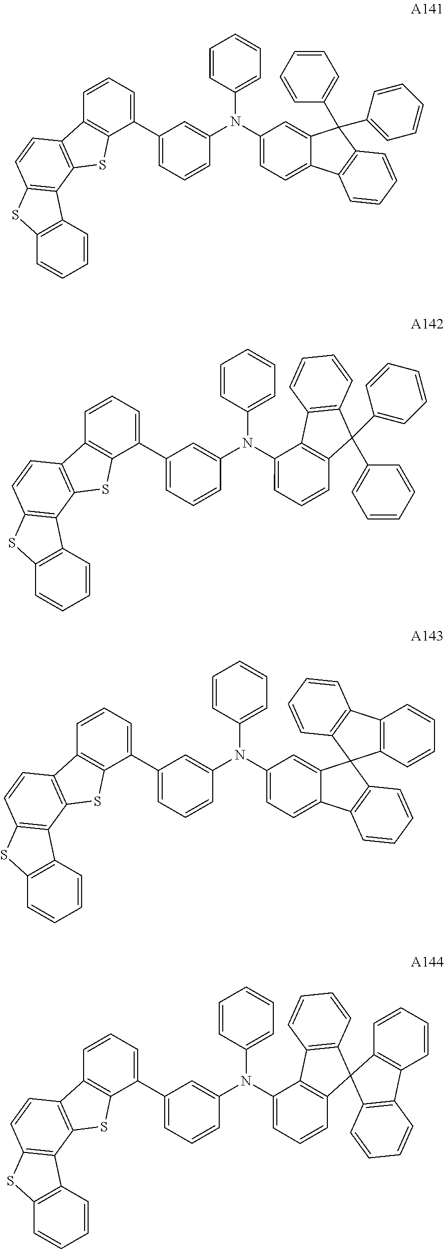 [SMILES: c1ccc(N(c2cccc(-c3cccc4c3sc3c4ccc4sc5ccccc5c43)c2)c2ccc3c(c2)C(c2ccccc2)(c2ccccc2)c2ccccc2-3)cc1 ; c1ccc(N(c2cccc(-c3cccc4c3sc3c4ccc4sc5ccccc5c43)c2)c2cccc3c2-c2ccccc2C3(c2ccccc2)c2ccccc2)cc1 ; c1ccc(N(c2cccc(-c3cccc4c3sc3c4ccc4sc5ccccc5c43)c2)c2ccc3c(c2)C2(c4ccccc4-c4ccccc42)c2ccccc2-3)cc1 ; c1ccc(N(c2cccc(-c3cccc4c3sc3c4ccc4sc5ccccc5c43)c2)c2cccc3c2-c2ccccc2C32c3ccccc3-c3ccccc32)cc1]

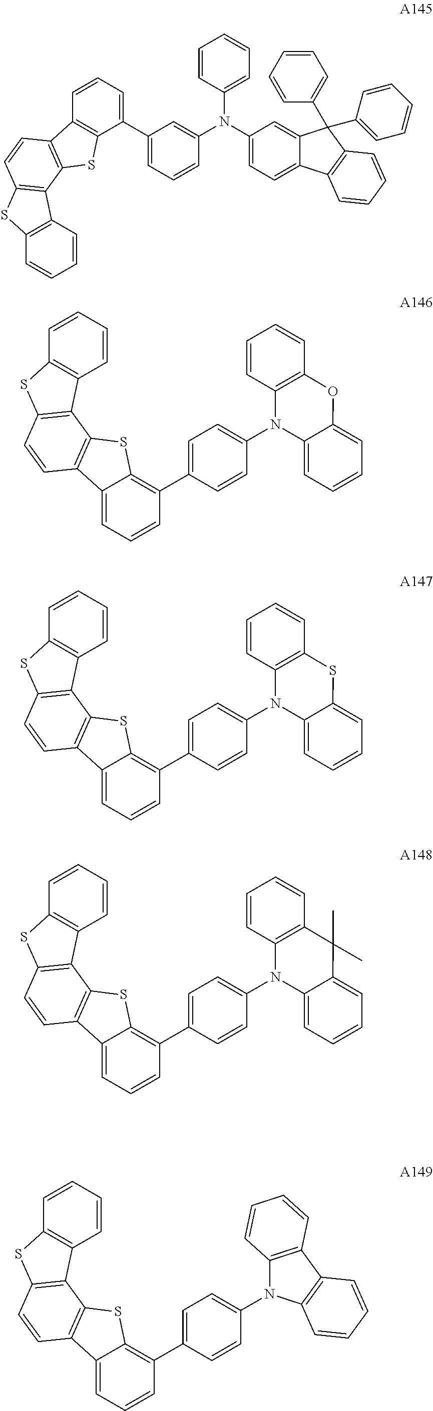

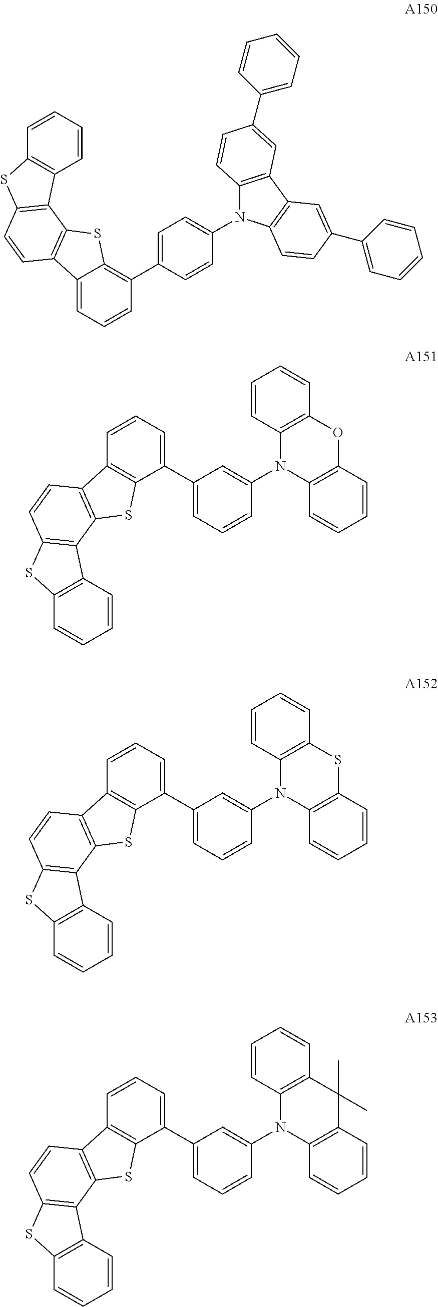 [SMILES: c1ccc(-c2ccc3c(c2)c2cc(-c4ccccc4)ccc2n3-c2ccc(-c3cccc4c3sc3c4ccc4sc5ccccc5c43)cc2)cc1 ; c1cc(-c2cccc3c2sc2c3ccc3sc4ccccc4c32)cc(N2c3ccccc3Oc3ccccc32)c1 ; c1cc(-c2cccc3c2sc2c3ccc3sc4ccccc4c32)cc(N2c3ccccc3Sc3ccccc32)c1 ; CC1(C)c2ccccc2N(c2cccc(-c3cccc4c3sc3c4ccc4sc5ccccc5c43)c2)c2ccccc21]

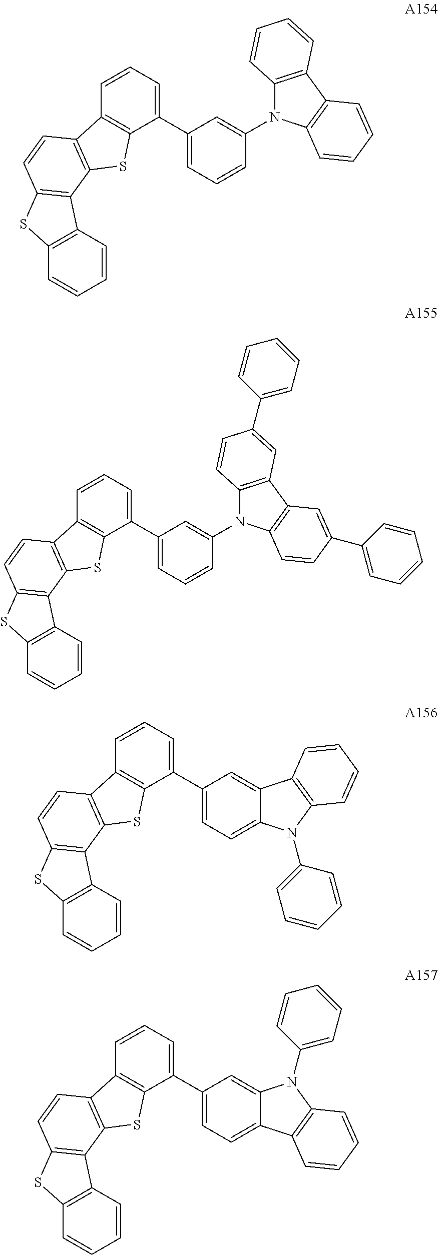 [SMILES: c1cc(-c2cccc3c2sc2c3ccc3sc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3sc3c4ccc4sc5ccccc5c43)c2)cc1 ; c1ccc(-n2c3ccccc3c3cc(-c4cccc5c4sc4c5ccc5sc6ccccc6c54)ccc32)cc1 ; c1ccc(-n2c3ccccc3c3ccc(-c4cccc5c4sc4c5ccc5sc6ccccc6c54)cc32)cc1]

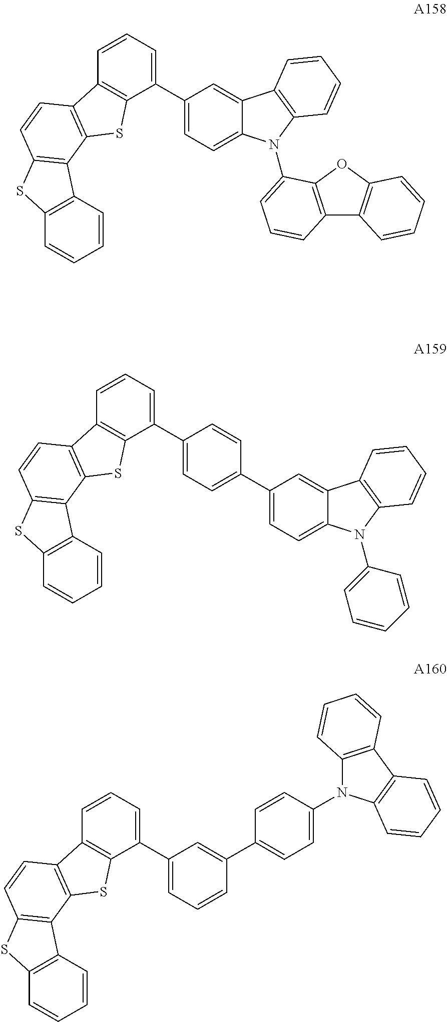 [SMILES: c1ccc2c(c1)oc1c(-n3c4ccccc4c4cc(-c5cccc6c5sc5c6ccc6sc7ccccc7c65)ccc43)cccc12 ; c1ccc(-n2c3ccccc3c3cc(-c4ccc(-c5cccc6c5sc5c6ccc6sc7ccccc7c65)cc4)ccc32)cc1 ; c1cc(-c2ccc(-n3c4ccccc4c4ccccc43)cc2)cc(-c2cccc3c2sc2c3ccc3sc4ccccc4c32)c1]

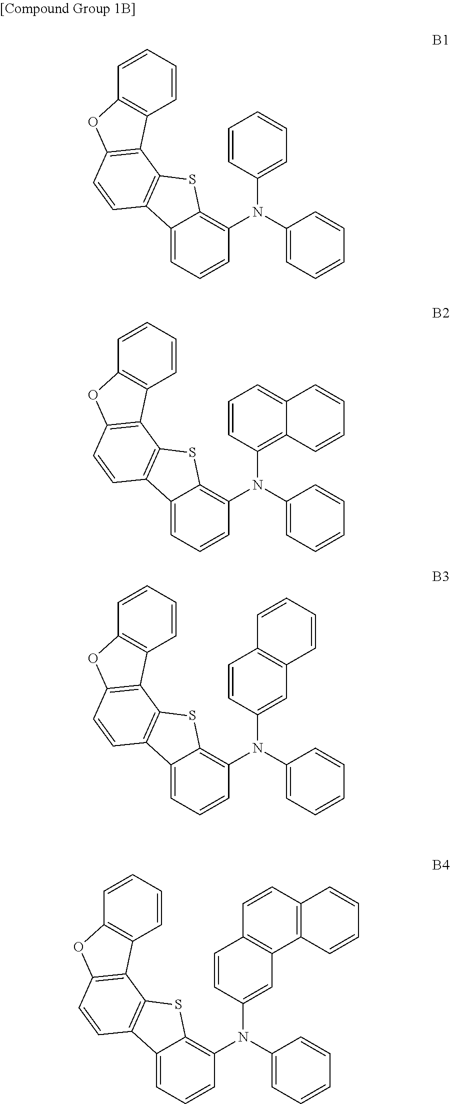 [SMILES: c1ccc(N(c2ccccc2)c2cccc3c2sc2c3ccc3oc4ccccc4c32)cc1 ; c1ccc(N(c2cccc3ccccc23)c2cccc3c2sc2c3ccc3oc4ccccc4c32)cc1 ; c1ccc(N(c2ccc3ccccc3c2)c2cccc3c2sc2c3ccc3oc4ccccc4c32)cc1 ; c1ccc(N(c2ccc3ccc4ccccc4c3c2)c2cccc3c2sc2c3ccc3oc4ccccc4c32)cc1]

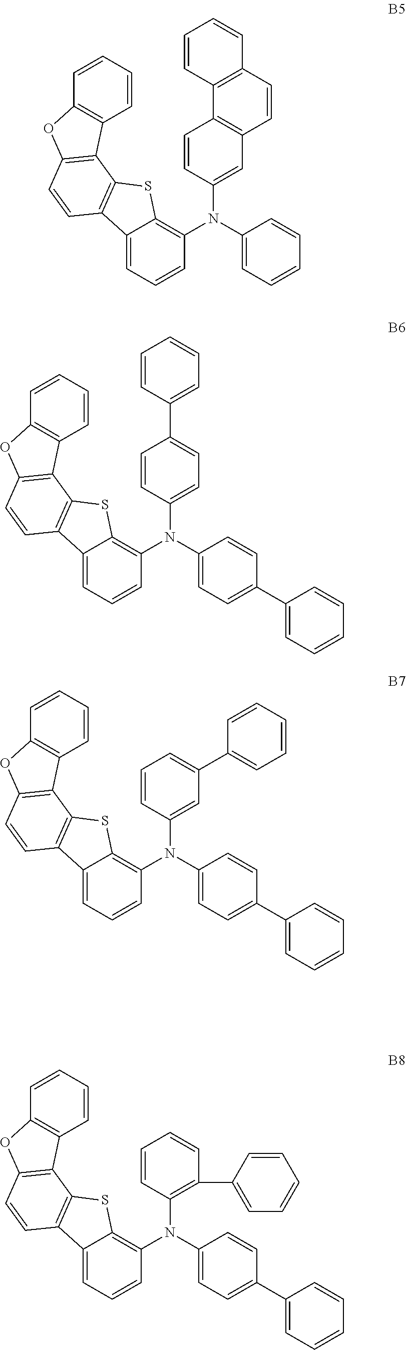 [SMILES: c1ccc(N(c2ccc3c(ccc4ccccc43)c2)c2cccc3c2sc2c3ccc3oc4ccccc4c32)cc1 ; c1ccc(-c2ccc(N(c3ccc(-c4ccccc4)cc3)c3cccc4c3sc3c4ccc4oc5ccccc5c43)cc2)cc1 ; c1ccc(-c2ccc(N(c3cccc(-c4ccccc4)c3)c3cccc4c3sc3c4ccc4oc5ccccc5c43)cc2)cc1 ; c1ccc(-c2ccc(N(c3ccccc3-c3ccccc3)c3cccc4c3sc3c4ccc4oc5ccccc5c43)cc2)cc1]

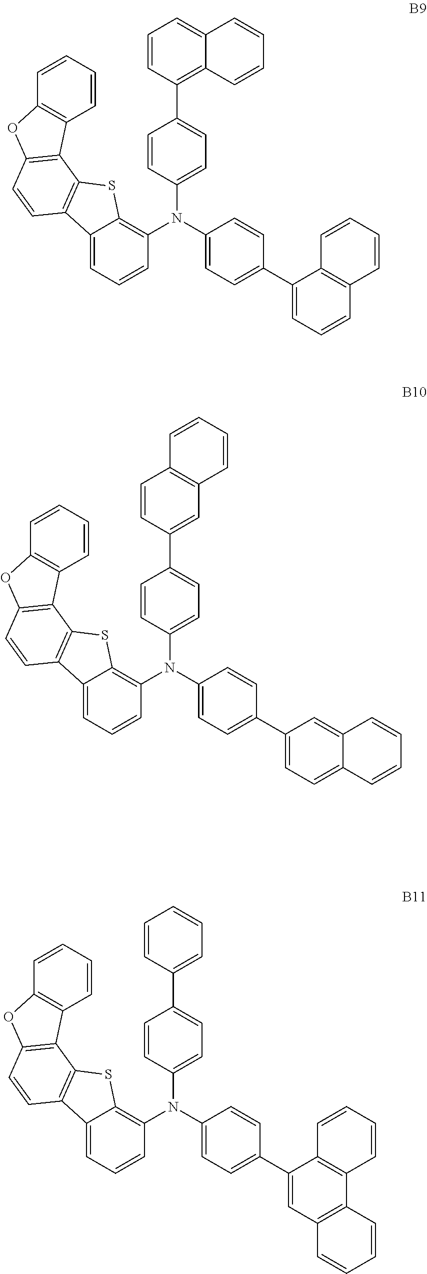 [SMILES: c1ccc2c(-c3ccc(N(c4ccc(-c5cccc6ccccc56)cc4)c4cccc5c4sc4c5ccc5oc6ccccc6c54)cc3)cccc2c1 ; c1ccc2cc(-c3ccc(N(c4ccc(-c5ccc6ccccc6c5)cc4)c4cccc5c4sc4c5ccc5oc6ccccc6c54)cc3)ccc2c1 ; c1ccc(-c2ccc(N(c3ccc(-c4cc5ccccc5c5ccccc45)cc3)c3cccc4c3sc3c4ccc4oc5ccccc5c43)cc2)cc1]

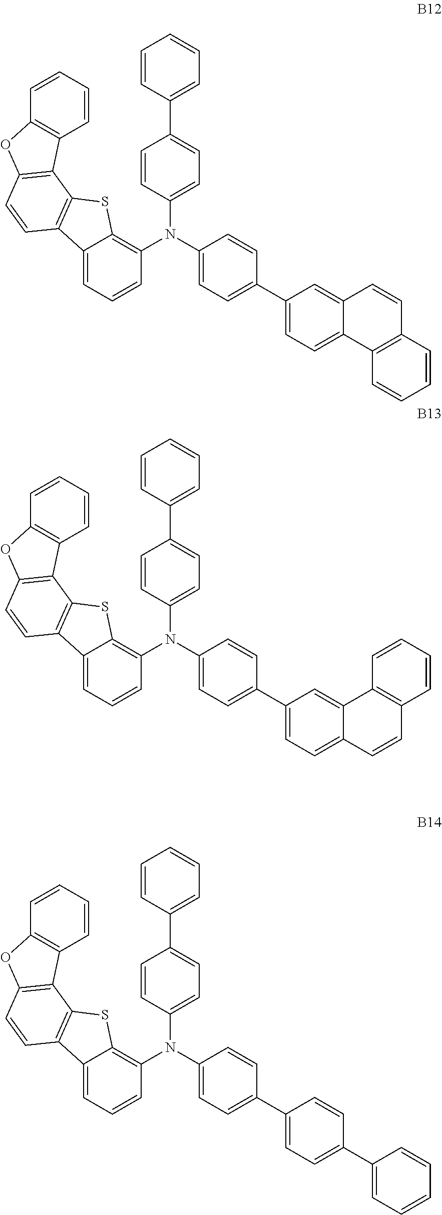 [SMILES: c1ccc(-c2ccc(N(c3ccc(-c4ccc5c(ccc6ccccc65)c4)cc3)c3cccc4c3sc3c4ccc4oc5ccccc5c43)cc2)cc1 ; c1ccc(-c2ccc(N(c3ccc(-c4ccc5ccc6ccccc6c5c4)cc3)c3cccc4c3sc3c4ccc4oc5ccccc5c43)cc2)cc1 ; c1ccc(-c2ccc(-c3ccc(N(c4ccc(-c5ccccc5)cc4)c4cccc5c4sc4c5ccc5oc6ccccc6c54)cc3)cc2)cc1]

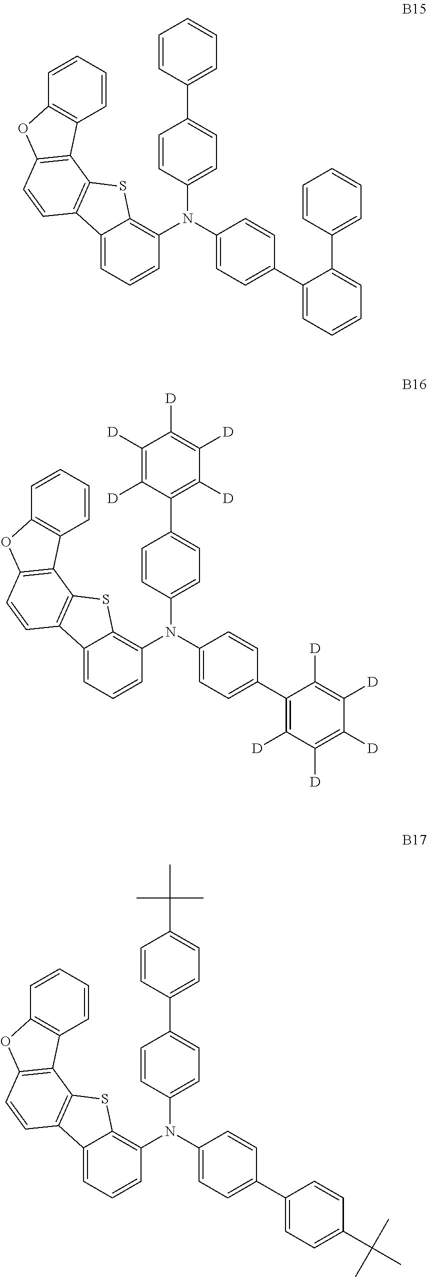 [SMILES: c1ccc(-c2ccc(N(c3ccc(-c4ccccc4-c4ccccc4)cc3)c3cccc4c3sc3c4ccc4oc5ccccc5c43)cc2)cc1 ; [2H]c1c([2H])c([2H])c(-c2ccc(N(c3ccc(-c4c([2H])c([2H])c([2H])c([2H])c4[2H])cc3)c3cccc4c3sc3c4ccc4oc5ccccc5c43)cc2)c([2H])c1[2H] ; CC(C)(C)c1ccc(-c2ccc(N(c3ccc(-c4ccc(C(C)(C)C)cc4)cc3)c3cccc4c3sc3c4ccc4oc5ccccc5c43)cc2)cc1]

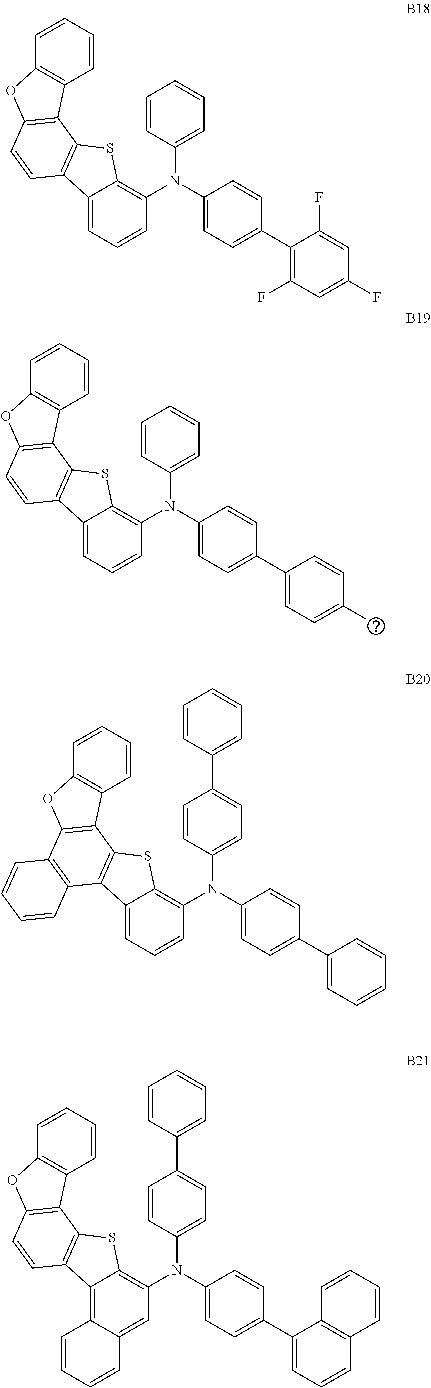 [SMILES: Fc1cc(F)c(-c2ccc(N(c3ccccc3)c3cccc4c3sc3c4ccc4oc5ccccc5c43)cc2)c(F)c1 ; Cc1ccc(-c2ccc(N(c3ccccc3)c3cccc4c3sc3c4ccc4oc5ccccc5c43)cc2)cc1 ; c1ccc(-c2ccc(N(c3ccc(-c4ccccc4)cc3)c3cccc4c3sc3c4c4ccccc4c4oc5ccccc5c43)cc2)cc1 ; c1ccc(-c2ccc(N(c3ccc(-c4cccc5ccccc45)cc3)c3cc4ccccc4c4c3sc3c4ccc4oc5ccccc5c43)cc2)cc1]

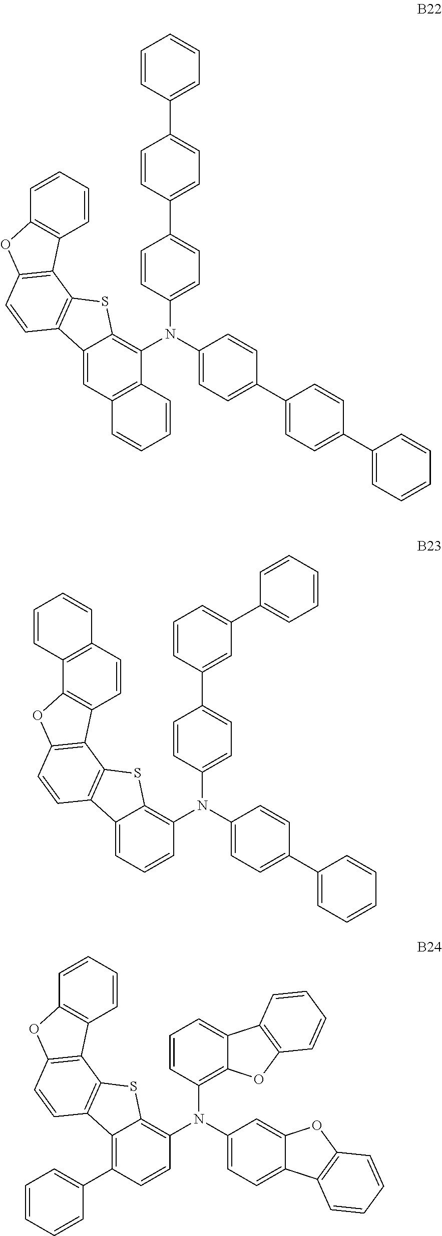 [SMILES: c1ccc(-c2ccc(-c3ccc(N(c4ccc(-c5ccc(-c6ccccc6)cc5)cc4)c4c5ccccc5cc5c4sc4c5ccc5oc6ccccc6c54)cc3)cc2)cc1 ; c1ccc(-c2ccc(N(c3ccc(-c4cccc(-c5ccccc5)c4)cc3)c3cccc4c3sc3c4ccc4oc5c6ccccc6ccc5c43)cc2)cc1 ; c1ccc(-c2ccc(N(c3ccc4c(c3)oc3ccccc34)c3cccc4c3oc3ccccc34)c3sc4c(ccc5oc6ccccc6c54)c23)cc1]

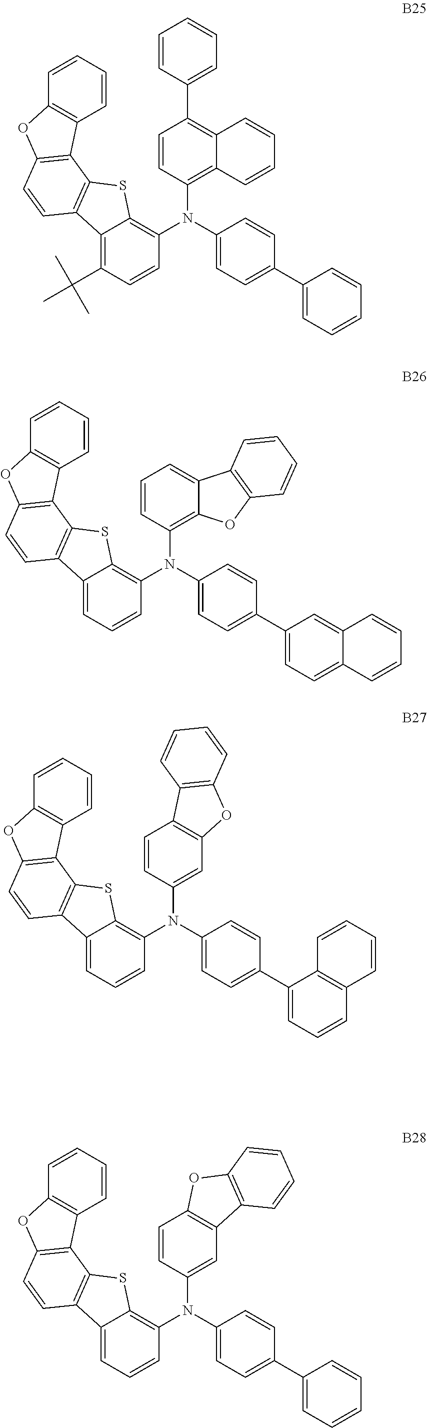 [SMILES: CC(C)(C)c1ccc(N(c2ccc(-c3ccccc3)cc2)c2ccc(-c3ccccc3)c3ccccc23)c2sc3c(ccc4oc5ccccc5c43)c12 ; c1ccc2cc(-c3ccc(N(c4cccc5c4oc4ccccc45)c4cccc5c4sc4c5ccc5oc6ccccc6c54)cc3)ccc2c1 ; c1ccc2c(-c3ccc(N(c4ccc5c(c4)oc4ccccc45)c4cccc5c4sc4c5ccc5oc6ccccc6c54)cc3)cccc2c1 ; c1ccc(-c2ccc(N(c3ccc4oc5ccccc5c4c3)c3cccc4c3sc3c4ccc4oc5ccccc5c43)cc2)cc1]

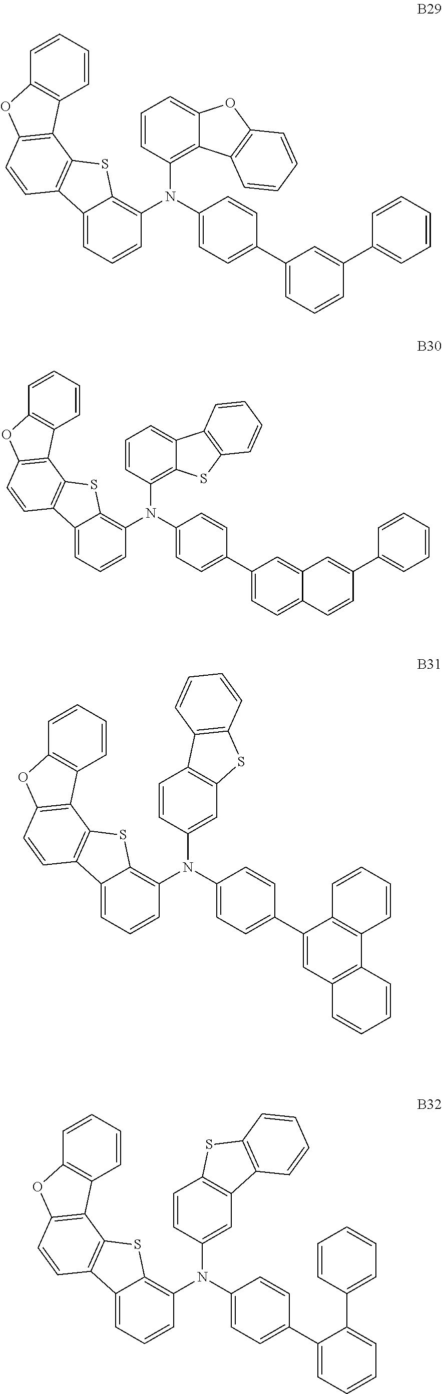 [SMILES: c1ccc(-c2cccc(-c3ccc(N(c4cccc5c4sc4c5ccc5oc6ccccc6c54)c4cccc5oc6ccccc6c45)cc3)c2)cc1 ; c1ccc(-c2ccc3ccc(-c4ccc(N(c5cccc6c5sc5ccccc56)c5cccc6c5sc5c6ccc6oc7ccccc7c65)cc4)cc3c2)cc1 ; c1ccc2c(c1)cc(-c1ccc(N(c3ccc4c(c3)sc3ccccc34)c3cccc4c3sc3c4ccc4oc5ccccc5c43)cc1)c1ccccc12 ; c1ccc(-c2ccccc2-c2ccc(N(c3ccc4sc5ccccc5c4c3)c3cccc4c3sc3c4ccc4oc5ccccc5c43)cc2)cc1]

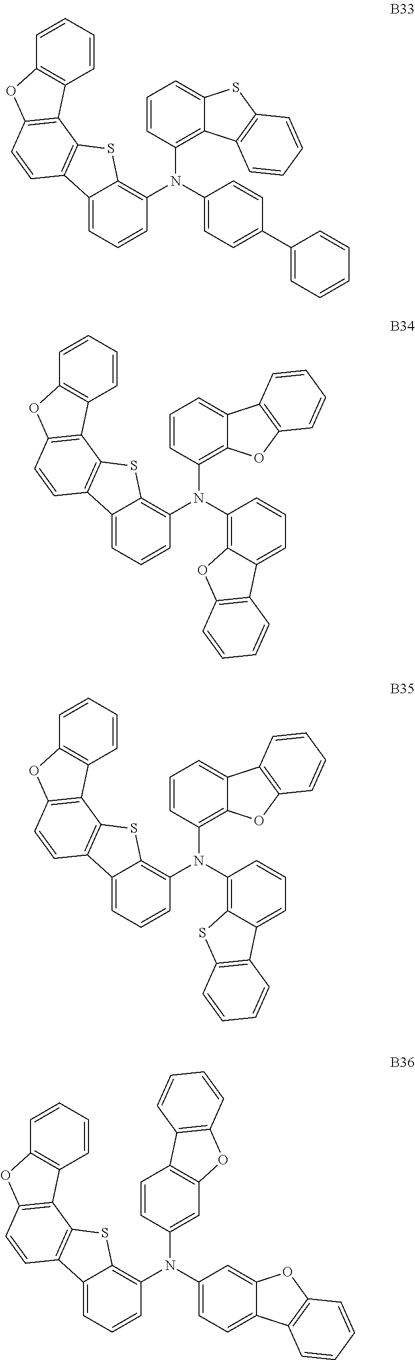 [SMILES: c1ccc(-c2ccc(N(c3cccc4c3sc3c4ccc4oc5ccccc5c43)c3cccc4sc5ccccc5c34)cc2)cc1 ; c1ccc2c(c1)oc1c(N(c3cccc4c3oc3ccccc34)c3cccc4c3sc3c4ccc4oc5ccccc5c43)cccc12 ; c1ccc2c(c1)oc1c(N(c3cccc4c3sc3ccccc34)c3cccc4c3sc3c4ccc4oc5ccccc5c43)cccc12 ; c1ccc2c(c1)oc1cc(N(c3ccc4c(c3)oc3ccccc34)c3cccc4c3sc3c4ccc4oc5ccccc5c43)ccc12]

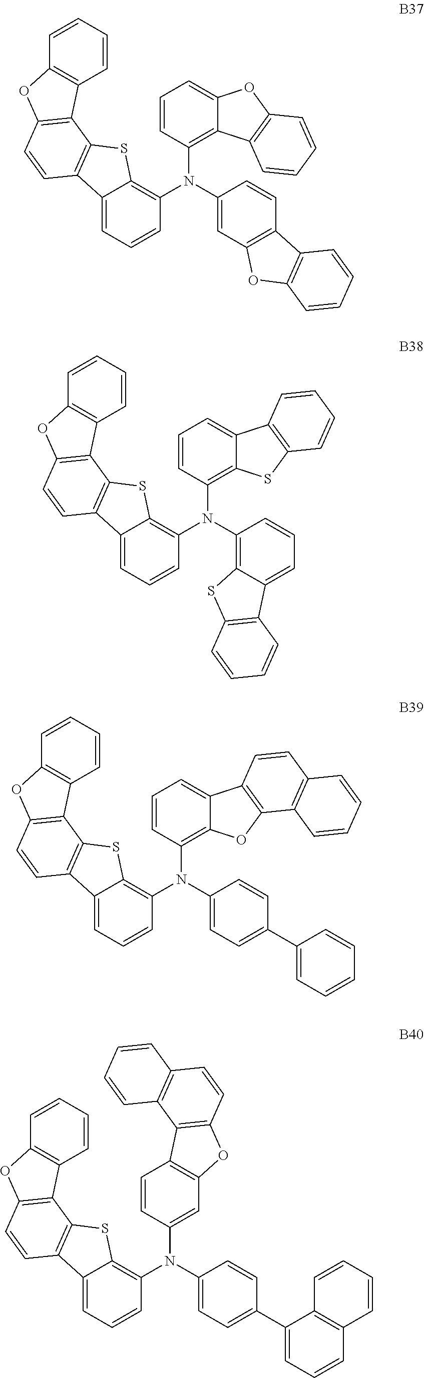 [SMILES: c1ccc2c(c1)oc1cc(N(c3cccc4c3sc3c4ccc4oc5ccccc5c43)c3cccc4oc5ccccc5c34)ccc12 ; c1ccc2c(c1)oc1ccc3c4cccc(N(c5cccc6c5sc5ccccc56)c5cccc6c5sc5ccccc56)c4sc3c12 ; c1ccc(-c2ccc(N(c3cccc4c3oc3c5ccccc5ccc43)c3cccc4c3sc3c4ccc4oc5ccccc5c43)cc2)cc1 ; c1ccc2c(-c3ccc(N(c4ccc5c(c4)oc4ccc6ccccc6c45)c4cccc5c4sc4c5ccc5oc6ccccc6c54)cc3)cccc2c1]

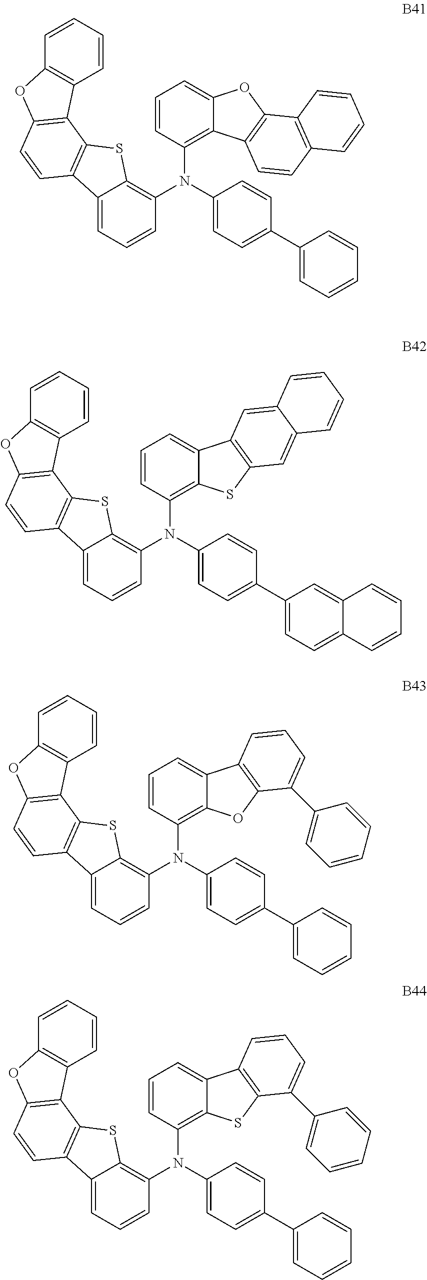 [SMILES: c1ccc(-c2ccc(N(c3cccc4c3sc3c4ccc4oc5ccccc5c43)c3cccc4oc5c6ccccc6ccc5c34)cc2)cc1 ; c1ccc2cc(-c3ccc(N(c4cccc5c4sc4cc6ccccc6cc45)c4cccc5c4sc4c5ccc5oc6ccccc6c54)cc3)ccc2c1 ; c1ccc(-c2ccc(N(c3cccc4c3oc3c(-c5ccccc5)cccc34)c3cccc4c3sc3c4ccc4oc5ccccc5c43)cc2)cc1 ; c1ccc(-c2ccc(N(c3cccc4c3sc3c(-c5ccccc5)cccc34)c3cccc4c3sc3c4ccc4oc5ccccc5c43)cc2)cc1]

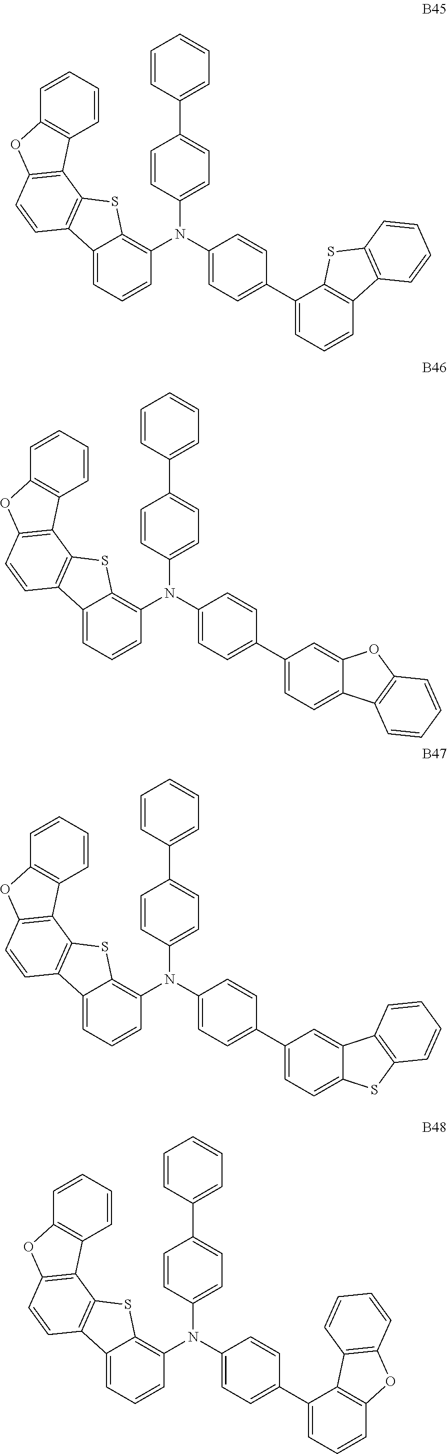 [SMILES: c1ccc(-c2ccc(N(c3ccc(-c4cccc5c4sc4ccccc45)cc3)c3cccc4c3sc3c4ccc4oc5ccccc5c43)cc2)cc1 ; c1ccc(-c2ccc(N(c3ccc(-c4ccc5c(c4)oc4ccccc45)cc3)c3cccc4c3sc3c4ccc4oc5ccccc5c43)cc2)cc1 ; c1ccc(-c2ccc(N(c3ccc(-c4ccc5sc6ccccc6c5c4)cc3)c3cccc4c3sc3c4ccc4oc5ccccc5c43)cc2)cc1 ; c1ccc(-c2ccc(N(c3ccc(-c4cccc5oc6ccccc6c45)cc3)c3cccc4c3sc3c4ccc4oc5ccccc5c43)cc2)cc1]

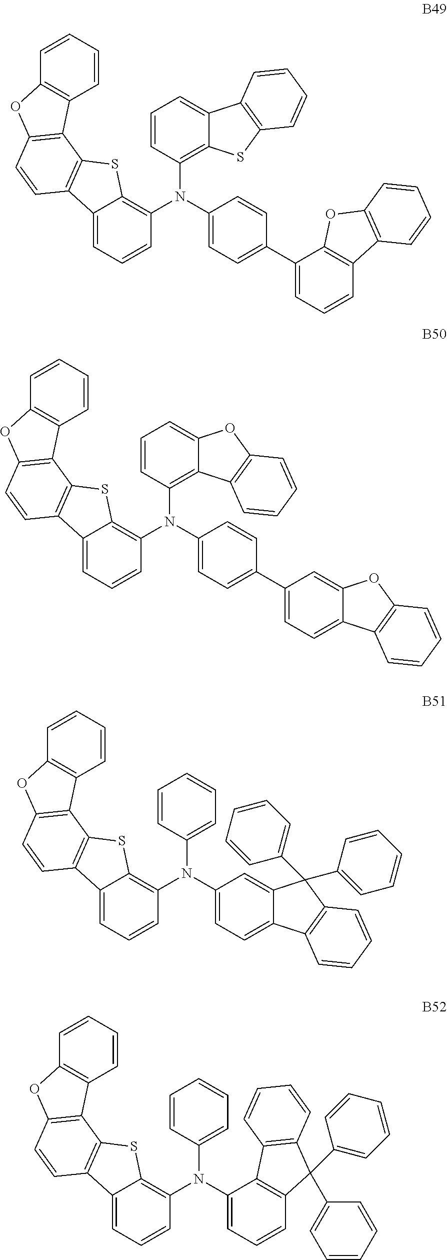 [SMILES: c1ccc2c(c1)oc1c(-c3ccc(N(c4cccc5c4sc4ccccc45)c4cccc5c4sc4c5ccc5oc6ccccc6c54)cc3)cccc12 ; c1ccc2c(c1)oc1cc(-c3ccc(N(c4cccc5c4sc4c5ccc5oc6ccccc6c54)c4cccc5oc6ccccc6c45)cc3)ccc12 ; c1ccc(N(c2ccc3c(c2)C(c2ccccc2)(c2ccccc2)c2ccccc2-3)c2cccc3c2sc2c3ccc3oc4ccccc4c32)cc1 ; c1ccc(N(c2cccc3c2-c2ccccc2C3(c2ccccc2)c2ccccc2)c2cccc3c2sc2c3ccc3oc4ccccc4c32)cc1]

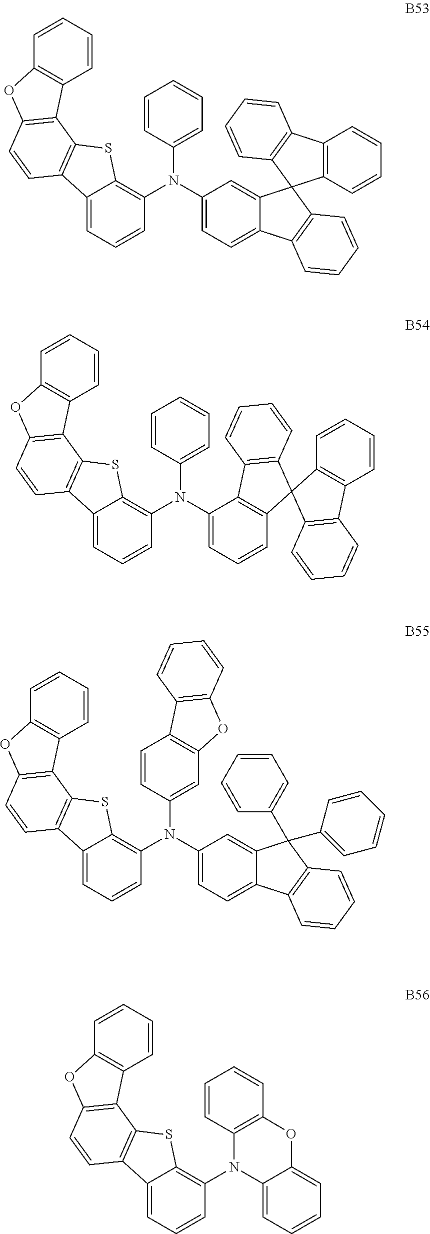 [SMILES: c1ccc(N(c2ccc3c(c2)C2(c4ccccc4-c4ccccc42)c2ccccc2-3)c2cccc3c2sc2c3ccc3oc4ccccc4c32)cc1 ; c1ccc(N(c2cccc3c2-c2ccccc2C32c3ccccc3-c3ccccc32)c2cccc3c2sc2c3ccc3oc4ccccc4c32)cc1 ; c1ccc(C2(c3ccccc3)c3ccccc3-c3ccc(N(c4ccc5c(c4)oc4ccccc45)c4cccc5c4sc4c5ccc5oc6ccccc6c54)cc32)cc1 ; c1ccc2c(c1)Oc1ccccc1N2c1cccc2c1sc1c2ccc2oc3ccccc3c21]

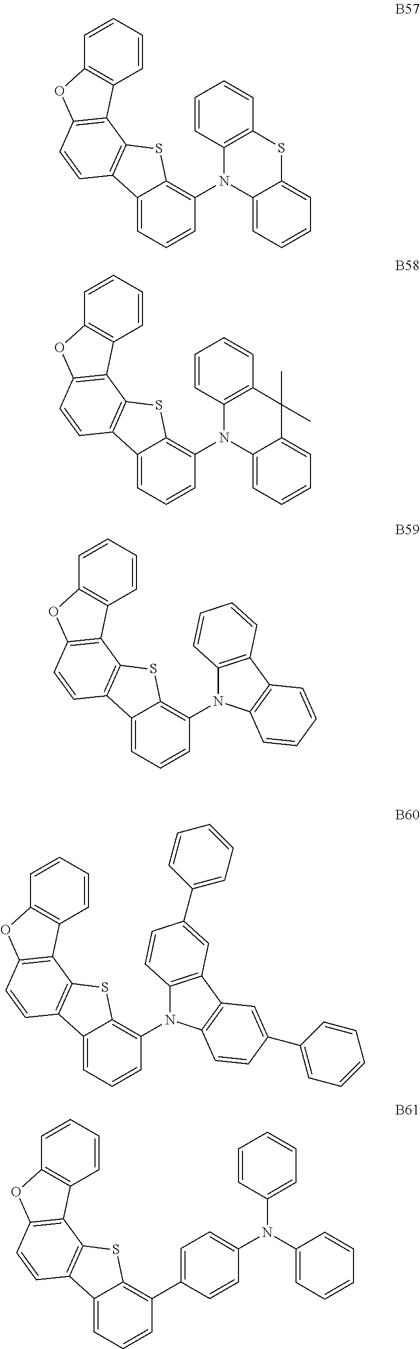 [SMILES: c1ccc2c(c1)Sc1ccccc1N2c1cccc2c1sc1c2ccc2oc3ccccc3c21 ; CC1(C)c2ccccc2N(c2cccc3c2sc2c3ccc3oc4ccccc4c32)c2ccccc21 ; c1ccc2c(c1)oc1ccc3c4cccc(-n5c6ccccc6c6ccccc65)c4sc3c12 ; c1ccc(-c2ccc3c(c2)c2cc(-c4ccccc4)ccc2n3-c2cccc3c2sc2c3ccc3oc4ccccc4c32)cc1 ; c1ccc(N(c2ccccc2)c2ccc(-c3cccc4c3sc3c4ccc4oc5ccccc5c43)cc2)cc1]

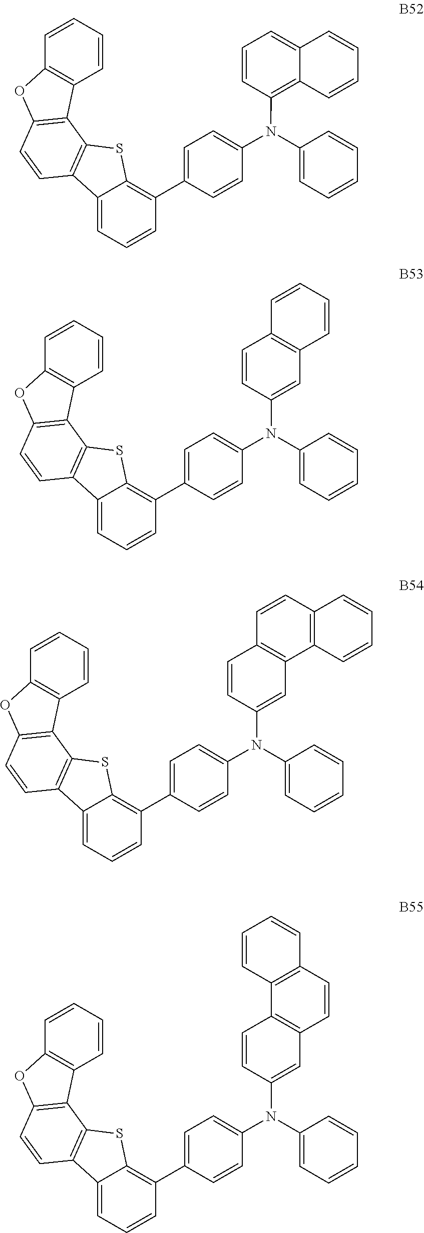 [SMILES: c1ccc(N(c2ccc(-c3cccc4c3sc3c4ccc4oc5ccccc5c43)cc2)c2cccc3ccccc23)cc1 ; c1ccc(N(c2ccc(-c3cccc4c3sc3c4ccc4oc5ccccc5c43)cc2)c2ccc3ccccc3c2)cc1 ; c1ccc(N(c2ccc(-c3cccc4c3sc3c4ccc4oc5ccccc5c43)cc2)c2ccc3ccc4ccccc4c3c2)cc1 ; c1ccc(N(c2ccc(-c3cccc4c3sc3c4ccc4oc5ccccc5c43)cc2)c2ccc3c(ccc4ccccc43)c2)cc1]

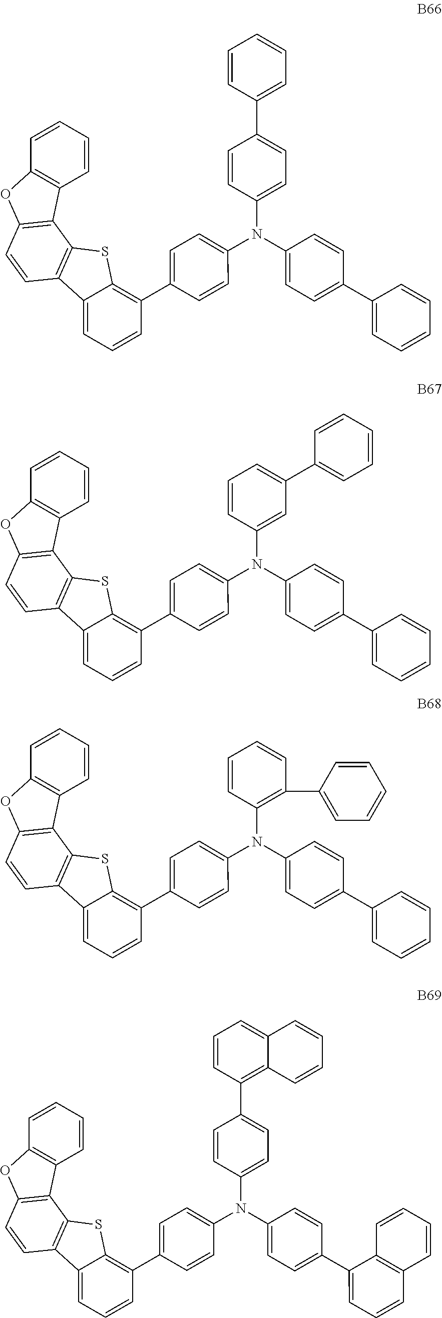 [SMILES: c1ccc(-c2ccc(N(c3ccc(-c4ccccc4)cc3)c3ccc(-c4cccc5c4sc4c5ccc5oc6ccccc6c54)cc3)cc2)cc1 ; c1ccc(-c2ccc(N(c3ccc(-c4cccc5c4sc4c5ccc5oc6ccccc6c54)cc3)c3cccc(-c4ccccc4)c3)cc2)cc1 ; c1ccc(-c2ccc(N(c3ccc(-c4cccc5c4sc4c5ccc5oc6ccccc6c54)cc3)c3ccccc3-c3ccccc3)cc2)cc1 ; c1ccc2c(-c3ccc(N(c4ccc(-c5cccc6ccccc56)cc4)c4ccc(-c5cccc6c5sc5c6ccc6oc7ccccc7c65)cc4)cc3)cccc2c1]

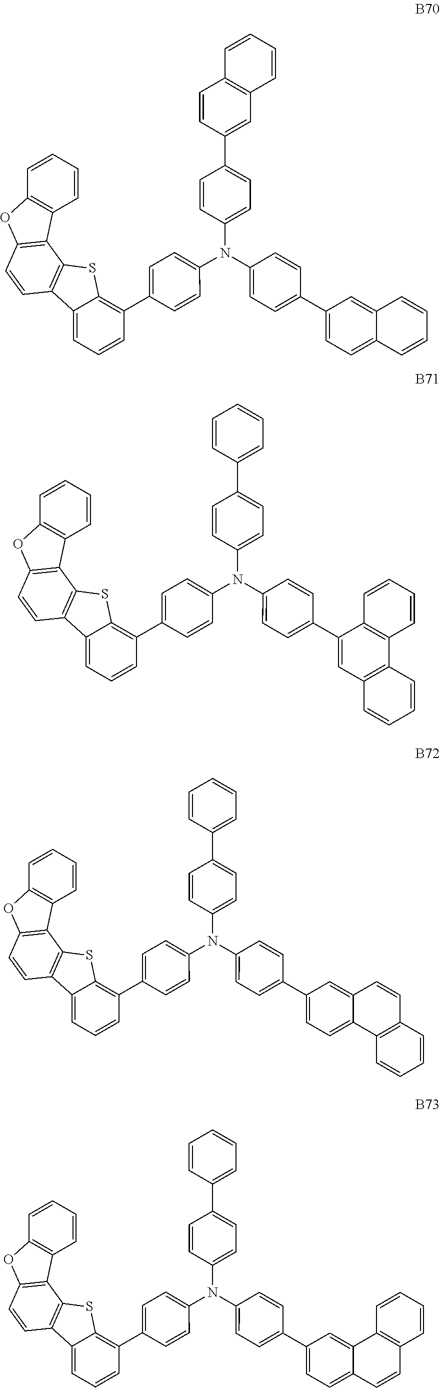 [SMILES: c1ccc2cc(-c3ccc(N(c4ccc(-c5ccc6ccccc6c5)cc4)c4ccc(-c5cccc6c5sc5c6ccc6oc7ccccc7c65)cc4)cc3)ccc2c1 ; c1ccc(-c2ccc(N(c3ccc(-c4cc5ccccc5c5ccccc45)cc3)c3ccc(-c4cccc5c4sc4c5ccc5oc6ccccc6c54)cc3)cc2)cc1 ; c1ccc(-c2ccc(N(c3ccc(-c4ccc5c(ccc6ccccc65)c4)cc3)c3ccc(-c4cccc5c4sc4c5ccc5oc6ccccc6c54)cc3)cc2)cc1 ; c1ccc(-c2ccc(N(c3ccc(-c4ccc5ccc6ccccc6c5c4)cc3)c3ccc(-c4cccc5c4sc4c5ccc5oc6ccccc6c54)cc3)cc2)cc1]

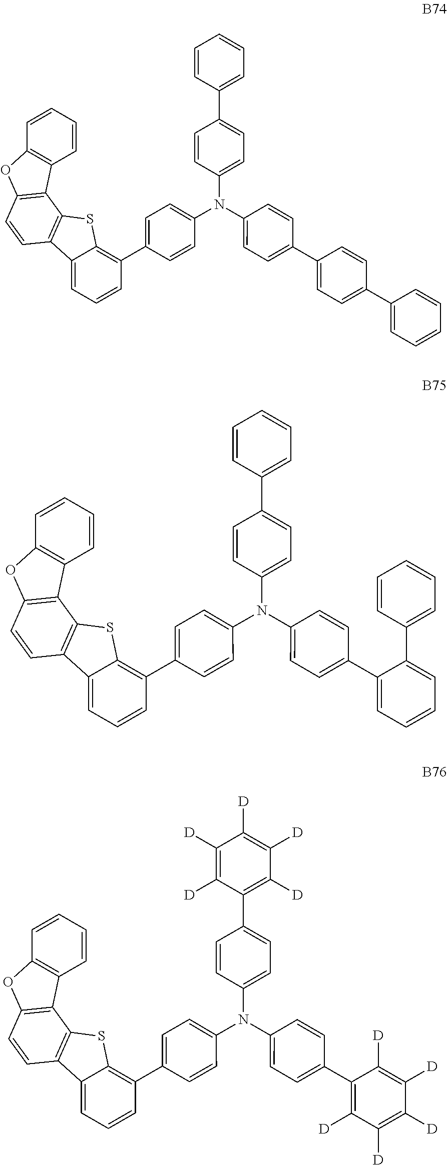 [SMILES: c1ccc(-c2ccc(-c3ccc(N(c4ccc(-c5ccccc5)cc4)c4ccc(-c5cccc6c5sc5c6ccc6oc7ccccc7c65)cc4)cc3)cc2)cc1 ; c1ccc(-c2ccc(N(c3ccc(-c4ccccc4-c4ccccc4)cc3)c3ccc(-c4cccc5c4sc4c5ccc5oc6ccccc6c54)cc3)cc2)cc1 ; [2H]c1c([2H])c([2H])c(-c2ccc(N(c3ccc(-c4c([2H])c([2H])c([2H])c([2H])c4[2H])cc3)c3ccc(-c4cccc5c4sc4c5ccc5oc6ccccc6c54)cc3)cc2)c([2H])c1[2H]]

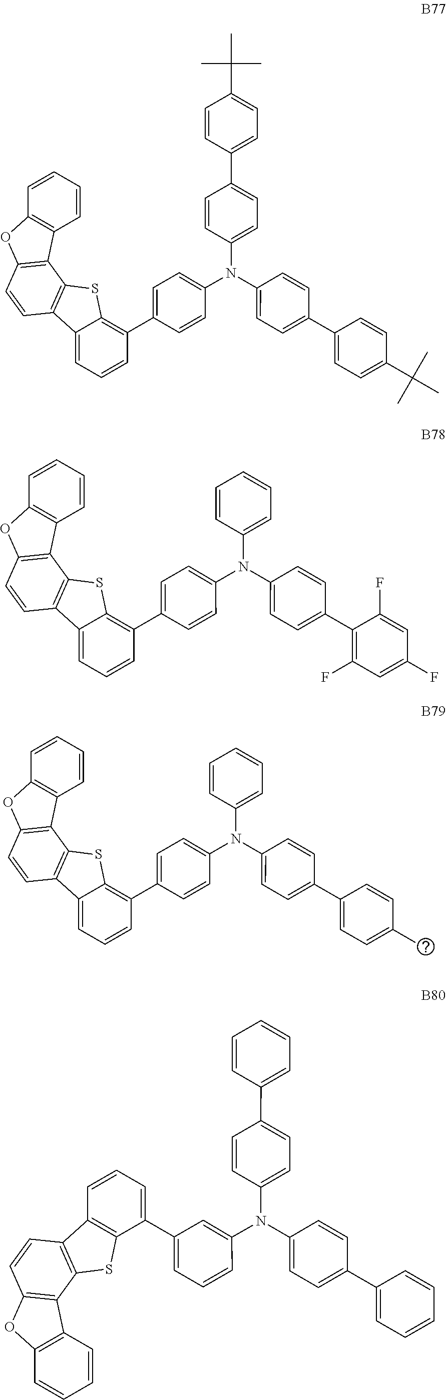 [SMILES: CC(C)(C)c1ccc(-c2ccc(N(c3ccc(-c4ccc(C(C)(C)C)cc4)cc3)c3ccc(-c4cccc5c4sc4c5ccc5oc6ccccc6c54)cc3)cc2)cc1 ; Fc1cc(F)c(-c2ccc(N(c3ccccc3)c3ccc(-c4cccc5c4sc4c5ccc5oc6ccccc6c54)cc3)cc2)c(F)c1 ; Cc1ccc(-c2ccc(N(c3ccccc3)c3ccc(-c4cccc5c4sc4c5ccc5oc6ccccc6c54)cc3)cc2)cc1 ; c1ccc(-c2ccc(N(c3ccc(-c4ccccc4)cc3)c3cccc(-c4cccc5c4sc4c5ccc5oc6ccccc6c54)c3)cc2)cc1]

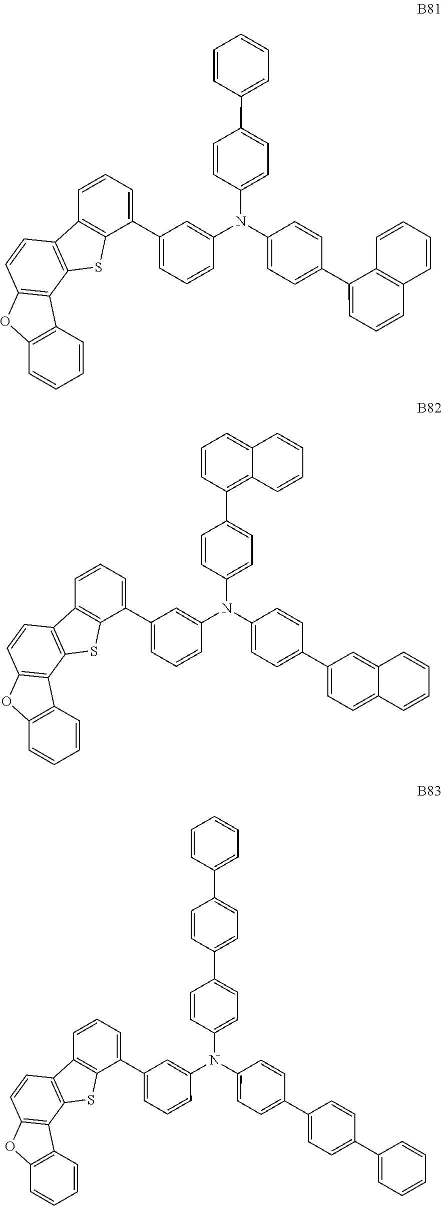 [SMILES: c1ccc(-c2ccc(N(c3ccc(-c4cccc5ccccc45)cc3)c3cccc(-c4cccc5c4sc4c5ccc5oc6ccccc6c54)c3)cc2)cc1 ; c1cc(-c2cccc3c2sc2c3ccc3oc4ccccc4c32)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6c5sc5c6ccc6oc7ccccc7c65)c4)cc3)cc2)cc1]

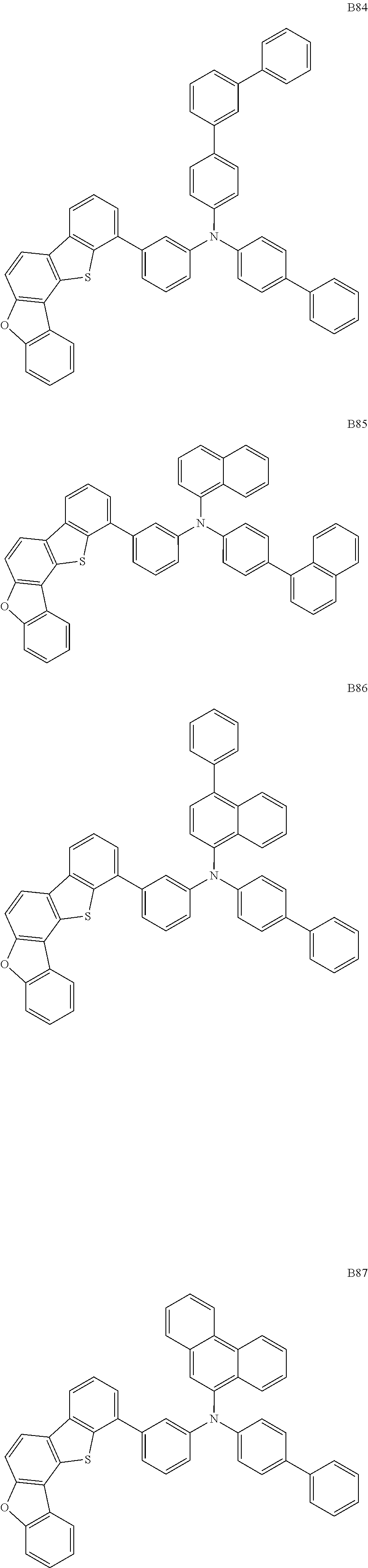 [SMILES: c1ccc(-c2ccc(N(c3ccc(-c4cccc(-c5ccccc5)c4)cc3)c3cccc(-c4cccc5c4sc4c5ccc5oc6ccccc6c54)c3)cc2)cc1 ; c1cc(-c2cccc3c2sc2c3ccc3oc4ccccc4c32)cc(N(c2ccc(-c3cccc4ccccc34)cc2)c2cccc3ccccc23)c1 ; c1ccc(-c2ccc(N(c3cccc(-c4cccc5c4sc4c5ccc5oc6ccccc6c54)c3)c3ccc(-c4ccccc4)c4ccccc34)cc2)cc1 ; c1ccc(-c2ccc(N(c3cccc(-c4cccc5c4sc4c5ccc5oc6ccccc6c54)c3)c3cc4ccccc4c4ccccc34)cc2)cc1]

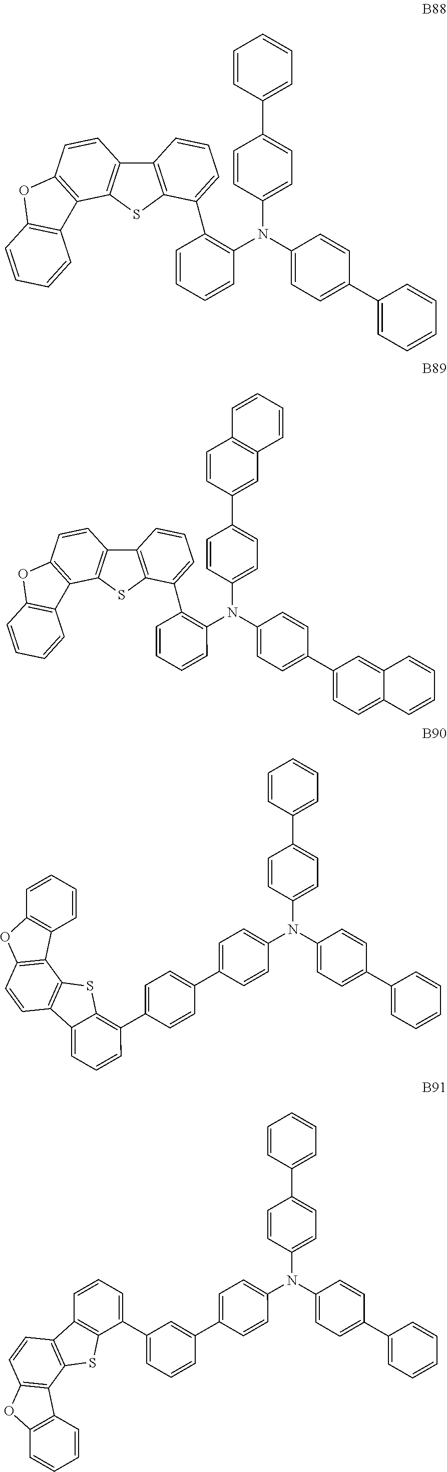 [SMILES: c1ccc(-c2ccc(N(c3ccc(-c4ccccc4)cc3)c3ccccc3-c3cccc4c3sc3c4ccc4oc5ccccc5c43)cc2)cc1 ; c1ccc(N(c2ccc(-c3ccc4ccccc4c3)cc2)c2ccc(-c3ccc4ccccc4c3)cc2)c(-c2cccc3c2sc2c3ccc3oc4ccccc4c32)c1 ; c1ccc(-c2ccc(N(c3ccc(-c4ccccc4)cc3)c3ccc(-c4ccc(-c5cccc6c5sc5c6ccc6oc7ccccc7c65)cc4)cc3)cc2)cc1 ; c1ccc(-c2ccc(N(c3ccc(-c4ccccc4)cc3)c3ccc(-c4cccc(-c5cccc6c5sc5c6ccc6oc7ccccc7c65)c4)cc3)cc2)cc1]

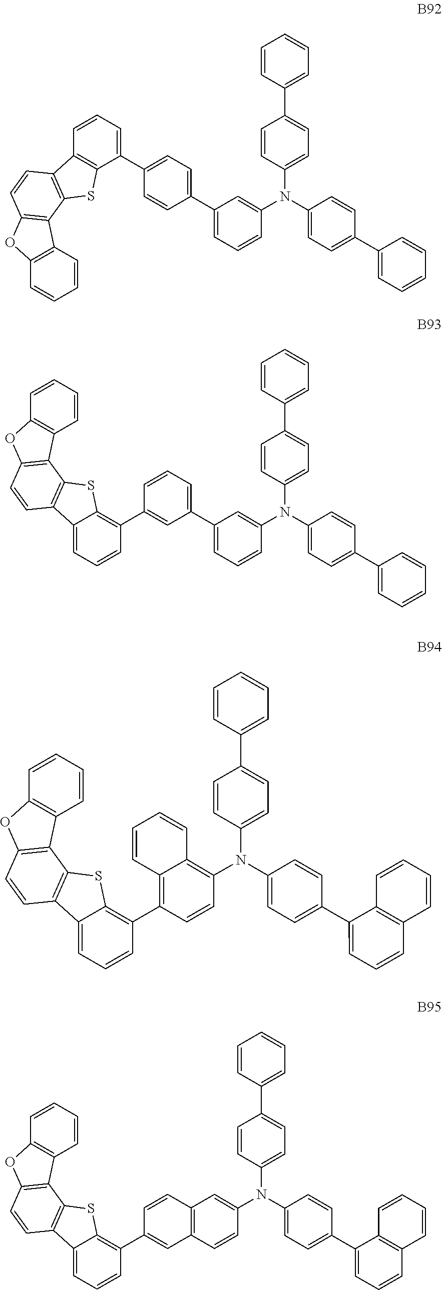 [SMILES: c1ccc(-c2ccc(N(c3ccc(-c4ccccc4)cc3)c3cccc(-c4ccc(-c5cccc6c5sc5c6ccc6oc7ccccc7c65)cc4)c3)cc2)cc1 ; c1ccc(-c2ccc(N(c3ccc(-c4ccccc4)cc3)c3cccc(-c4cccc(-c5cccc6c5sc5c6ccc6oc7ccccc7c65)c4)c3)cc2)cc1 ; c1ccc(-c2ccc(N(c3ccc(-c4cccc5ccccc45)cc3)c3ccc(-c4cccc5c4sc4c5ccc5oc6ccccc6c54)c4ccccc34)cc2)cc1 ; c1ccc(-c2ccc(N(c3ccc(-c4cccc5ccccc45)cc3)c3ccc4cc(-c5cccc6c5sc5c6ccc6oc7ccccc7c65)ccc4c3)cc2)cc1]

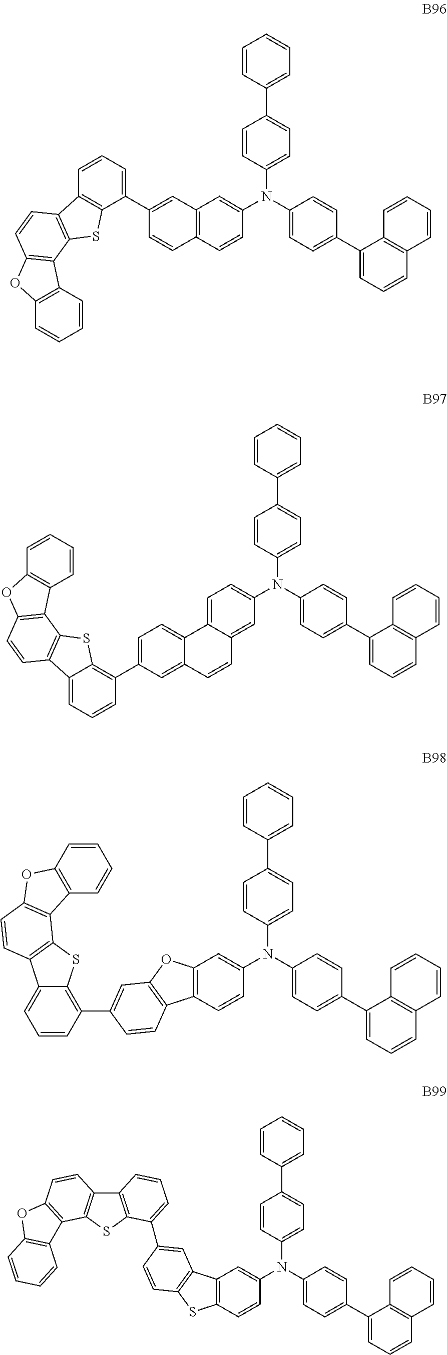 [SMILES: c1ccc(-c2ccc(N(c3ccc(-c4cccc5ccccc45)cc3)c3ccc4ccc(-c5cccc6c5sc5c6ccc6oc7ccccc7c65)cc4c3)cc2)cc1 ; c1ccc(-c2ccc(N(c3ccc(-c4cccc5ccccc45)cc3)c3ccc4c(ccc5cc(-c6cccc7c6sc6c7ccc7oc8ccccc8c76)ccc54)c3)cc2)cc1 ; c1ccc(-c2ccc(N(c3ccc(-c4cccc5ccccc45)cc3)c3ccc4c(c3)oc3cc(-c5cccc6c5sc5c6ccc6oc7ccccc7c65)ccc34)cc2)cc1 ; c1ccc(-c2ccc(N(c3ccc(-c4cccc5ccccc45)cc3)c3ccc4sc5ccc(-c6cccc7c6sc6c7ccc7oc8ccccc8c76)cc5c4c3)cc2)cc1]

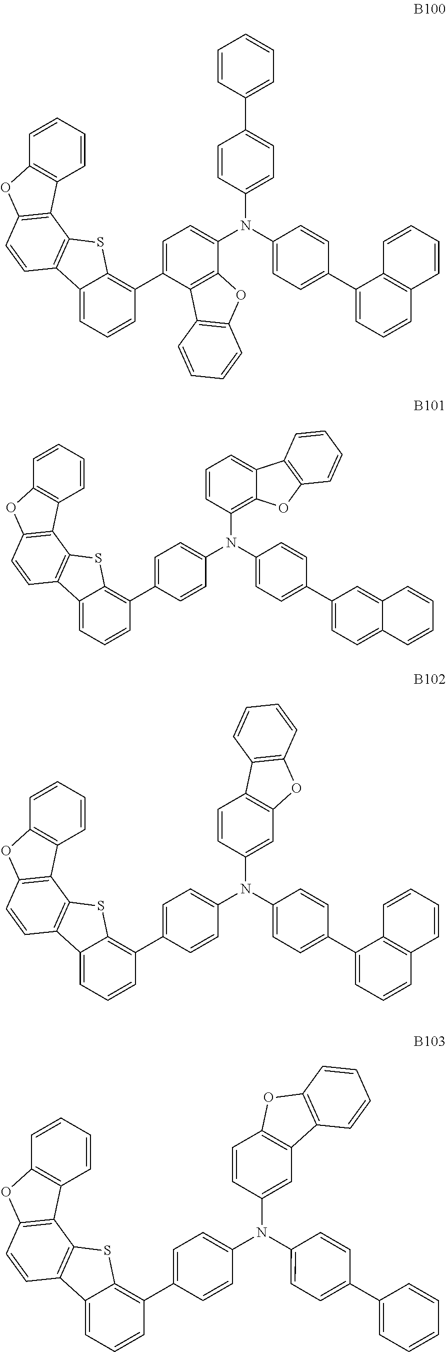 [SMILES: c1ccc(-c2ccc(N(c3ccc(-c4cccc5ccccc45)cc3)c3ccc(-c4cccc5c4sc4c5ccc5oc6ccccc6c54)c4c3oc3ccccc34)cc2)cc1 ; c1ccc2cc(-c3ccc(N(c4ccc(-c5cccc6c5sc5c6ccc6oc7ccccc7c65)cc4)c4cccc5c4oc4ccccc45)cc3)ccc2c1 ; c1ccc2c(-c3ccc(N(c4ccc(-c5cccc6c5sc5c6ccc6oc7ccccc7c65)cc4)c4ccc5c(c4)oc4ccccc45)cc3)cccc2c1 ; c1ccc(-c2ccc(N(c3ccc(-c4cccc5c4sc4c5ccc5oc6ccccc6c54)cc3)c3ccc4oc5ccccc5c4c3)cc2)cc1]

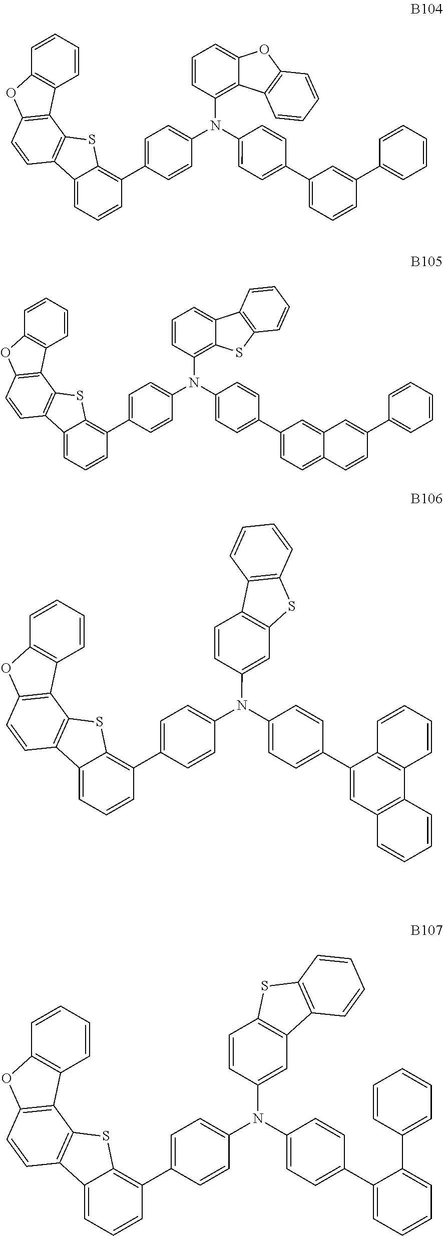 [SMILES: c1ccc(-c2cccc(-c3ccc(N(c4ccc(-c5cccc6c5sc5c6ccc6oc7ccccc7c65)cc4)c4cccc5oc6ccccc6c45)cc3)c2)cc1 ; c1ccc(-c2ccc3ccc(-c4ccc(N(c5ccc(-c6cccc7c6sc6c7ccc7oc8ccccc8c76)cc5)c5cccc6c5sc5ccccc56)cc4)cc3c2)cc1 ; c1ccc2c(c1)cc(-c1ccc(N(c3ccc(-c4cccc5c4sc4c5ccc5oc6ccccc6c54)cc3)c3ccc4c(c3)sc3ccccc34)cc1)c1ccccc12 ; c1ccc(-c2ccccc2-c2ccc(N(c3ccc(-c4cccc5c4sc4c5ccc5oc6ccccc6c54)cc3)c3ccc4sc5ccccc5c4c3)cc2)cc1]

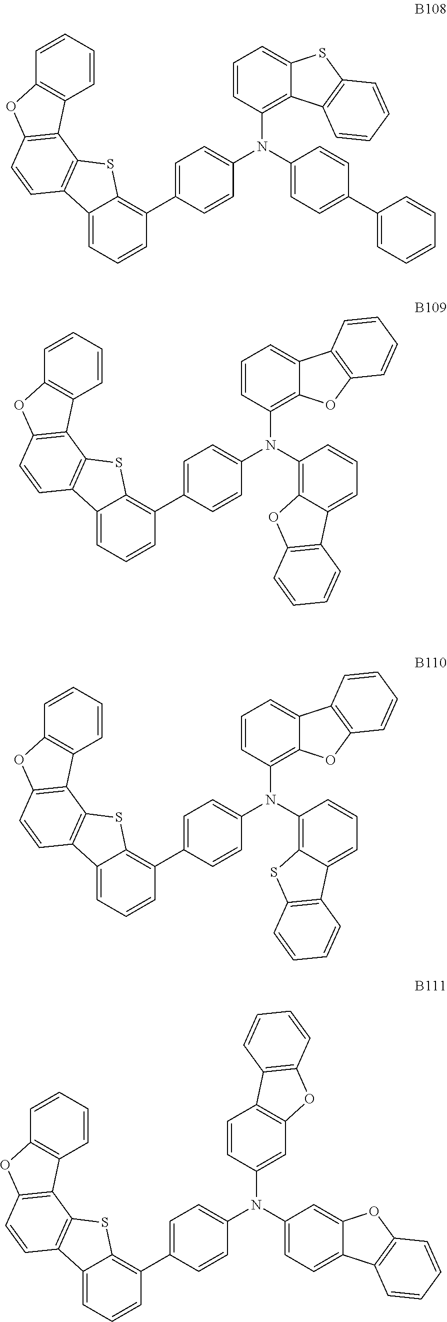 [SMILES: c1ccc(-c2ccc(N(c3ccc(-c4cccc5c4sc4c5ccc5oc6ccccc6c54)cc3)c3cccc4sc5ccccc5c34)cc2)cc1 ; c1ccc2c(c1)oc1c(N(c3ccc(-c4cccc5c4sc4c5ccc5oc6ccccc6c54)cc3)c3cccc4c3oc3ccccc34)cccc12 ; c1ccc2c(c1)oc1c(N(c3ccc(-c4cccc5c4sc4c5ccc5oc6ccccc6c54)cc3)c3cccc4c3sc3ccccc34)cccc12 ; c1ccc2c(c1)oc1cc(N(c3ccc(-c4cccc5c4sc4c5ccc5oc6ccccc6c54)cc3)c3ccc4c(c3)oc3ccccc34)ccc12]

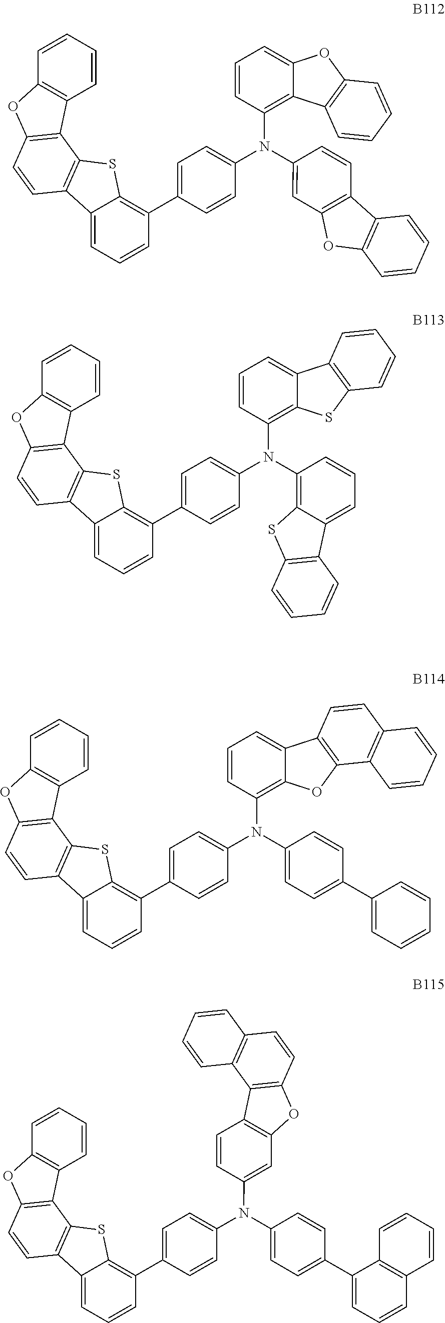 [SMILES: c1ccc2c(c1)oc1cc(N(c3ccc(-c4cccc5c4sc4c5ccc5oc6ccccc6c54)cc3)c3cccc4oc5ccccc5c34)ccc12 ; c1ccc2c(c1)oc1ccc3c4cccc(-c5ccc(N(c6cccc7c6sc6ccccc67)c6cccc7c6sc6ccccc67)cc5)c4sc3c12 ; c1ccc(-c2ccc(N(c3ccc(-c4cccc5c4sc4c5ccc5oc6ccccc6c54)cc3)c3cccc4c3oc3c5ccccc5ccc43)cc2)cc1 ; c1ccc2c(-c3ccc(N(c4ccc(-c5cccc6c5sc5c6ccc6oc7ccccc7c65)cc4)c4ccc5c(c4)oc4ccc6ccccc6c45)cc3)cccc2c1]

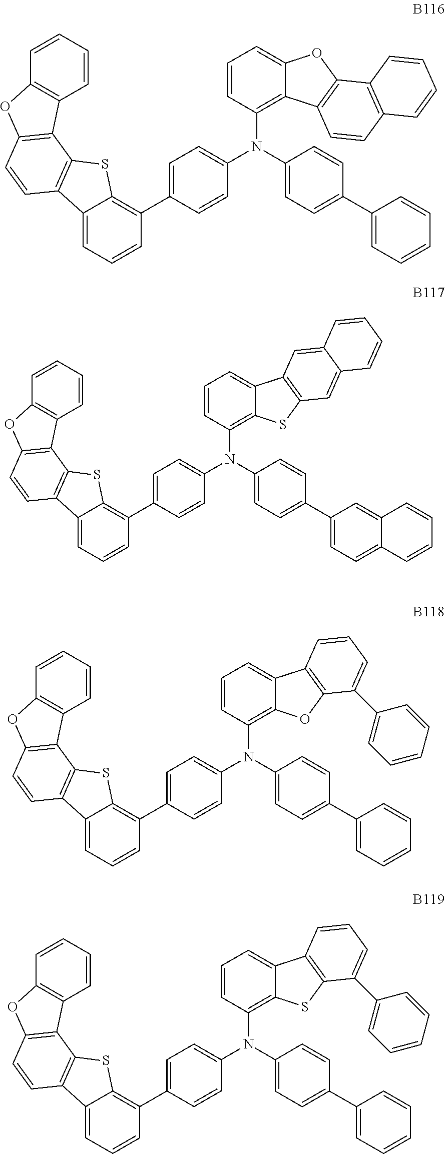 [SMILES: c1ccc(-c2ccc(N(c3ccc(-c4cccc5c4sc4c5ccc5oc6ccccc6c54)cc3)c3cccc4oc5c6ccccc6ccc5c34)cc2)cc1 ; c1ccc2cc(-c3ccc(N(c4ccc(-c5cccc6c5sc5c6ccc6oc7ccccc7c65)cc4)c4cccc5c4sc4cc6ccccc6cc45)cc3)ccc2c1 ; c1ccc(-c2ccc(N(c3ccc(-c4cccc5c4sc4c5ccc5oc6ccccc6c54)cc3)c3cccc4c3oc3c(-c5ccccc5)cccc34)cc2)cc1 ; c1ccc(-c2ccc(N(c3ccc(-c4cccc5c4sc4c5ccc5oc6ccccc6c54)cc3)c3cccc4c3sc3c(-c5ccccc5)cccc34)cc2)cc1]

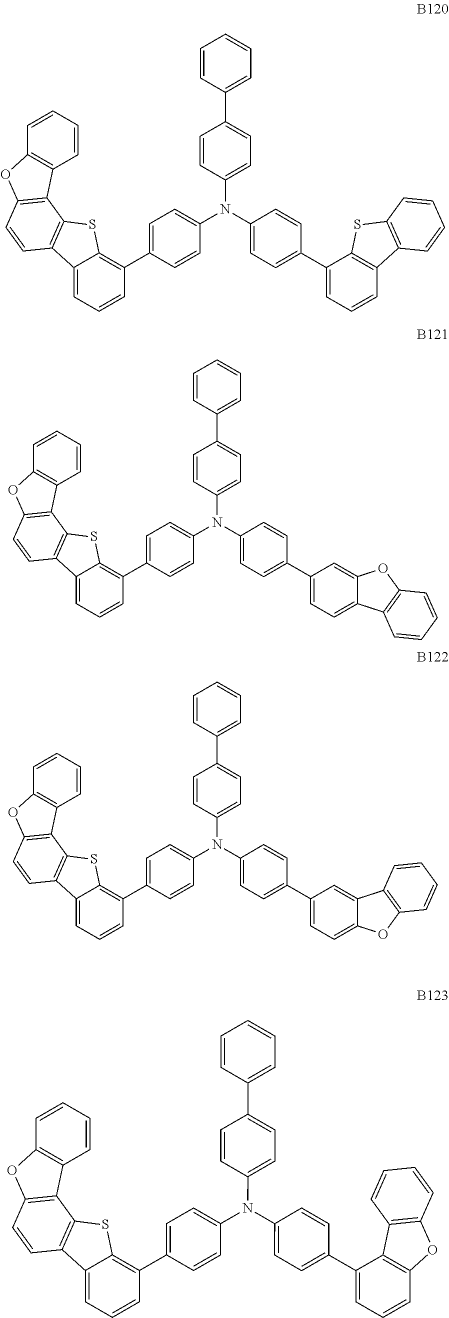 [SMILES: c1ccc(-c2ccc(N(c3ccc(-c4cccc5c4sc4ccccc45)cc3)c3ccc(-c4cccc5c4sc4c5ccc5oc6ccccc6c54)cc3)cc2)cc1 ; c1ccc(-c2ccc(N(c3ccc(-c4ccc5c(c4)oc4ccccc45)cc3)c3ccc(-c4cccc5c4sc4c5ccc5oc6ccccc6c54)cc3)cc2)cc1 ; c1ccc(-c2ccc(N(c3ccc(-c4ccc5oc6ccccc6c5c4)cc3)c3ccc(-c4cccc5c4sc4c5ccc5oc6ccccc6c54)cc3)cc2)cc1 ; c1ccc(-c2ccc(N(c3ccc(-c4cccc5c4sc4c5ccc5oc6ccccc6c54)cc3)c3ccc(-c4cccc5oc6ccccc6c45)cc3)cc2)cc1]

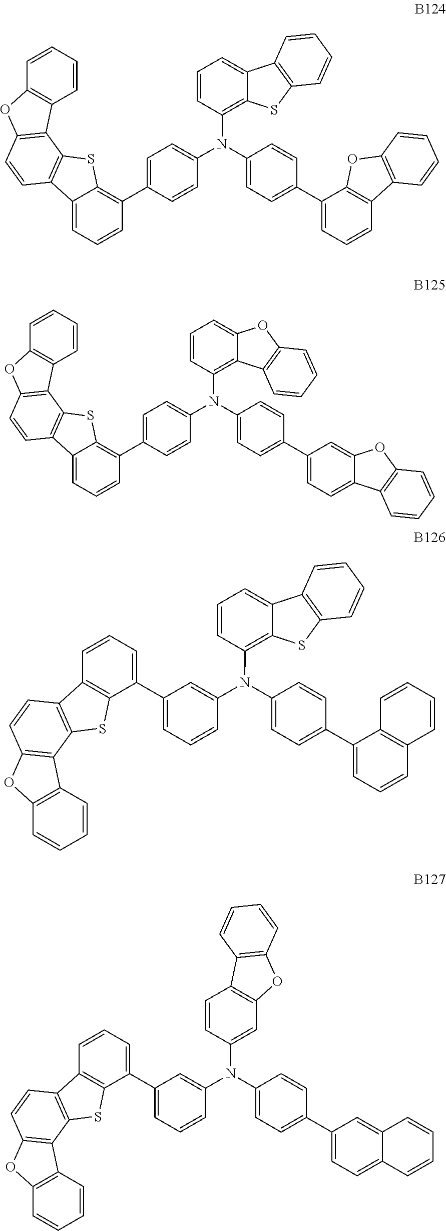 [SMILES: c1ccc2c(c1)oc1c(-c3ccc(N(c4ccc(-c5cccc6c5sc5c6ccc6oc7ccccc7c65)cc4)c4cccc5c4sc4ccccc45)cc3)cccc12 ; c1ccc2c(c1)oc1cc(-c3ccc(N(c4ccc(-c5cccc6c5sc5c6ccc6oc7ccccc7c65)cc4)c4cccc5oc6ccccc6c45)cc3)ccc12 ; c1cc(-c2cccc3c2sc2c3ccc3oc4ccccc4c32)cc(N(c2ccc(-c3cccc4ccccc34)cc2)c2cccc3c2sc2ccccc23)c1 ; c1cc(-c2cccc3c2sc2c3ccc3oc4ccccc4c32)cc(N(c2ccc(-c3ccc4ccccc4c3)cc2)c2ccc3c(c2)oc2ccccc23)c1]

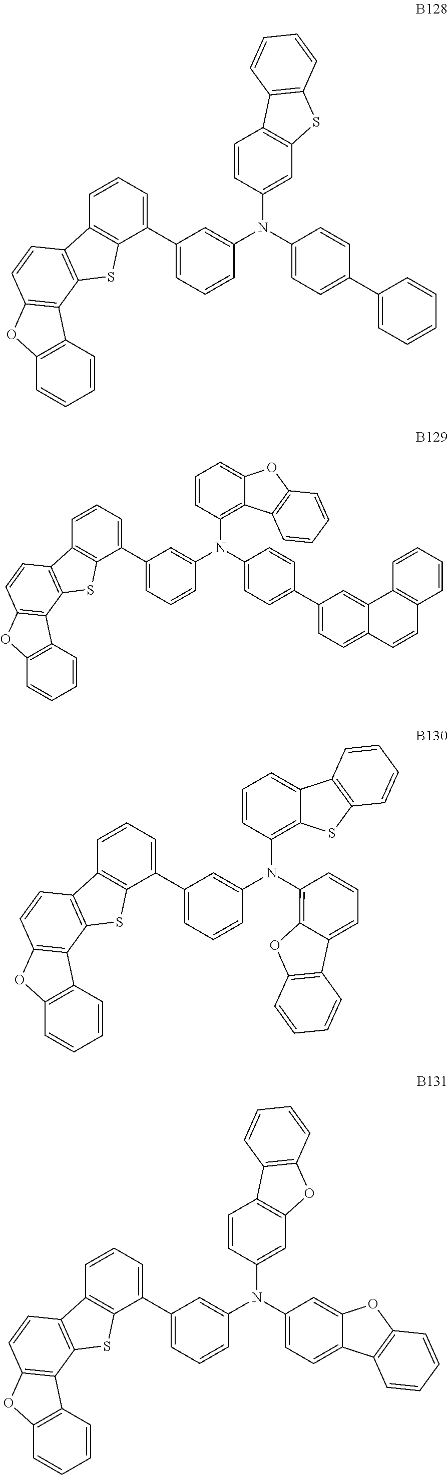 [SMILES: c1ccc(-c2ccc(N(c3cccc(-c4cccc5c4sc4c5ccc5oc6ccccc6c54)c3)c3ccc4c(c3)sc3ccccc34)cc2)cc1 ; c1cc(-c2cccc3c2sc2c3ccc3oc4ccccc4c32)cc(N(c2ccc(-c3ccc4ccc5ccccc5c4c3)cc2)c2cccc3oc4ccccc4c23)c1 ; c1cc(-c2cccc3c2sc2c3ccc3oc4ccccc4c32)cc(N(c2cccc3c2oc2ccccc23)c2cccc3c2sc2ccccc23)c1 ; c1cc(-c2cccc3c2sc2c3ccc3oc4ccccc4c32)cc(N(c2ccc3c(c2)oc2ccccc23)c2ccc3c(c2)oc2ccccc23)c1]

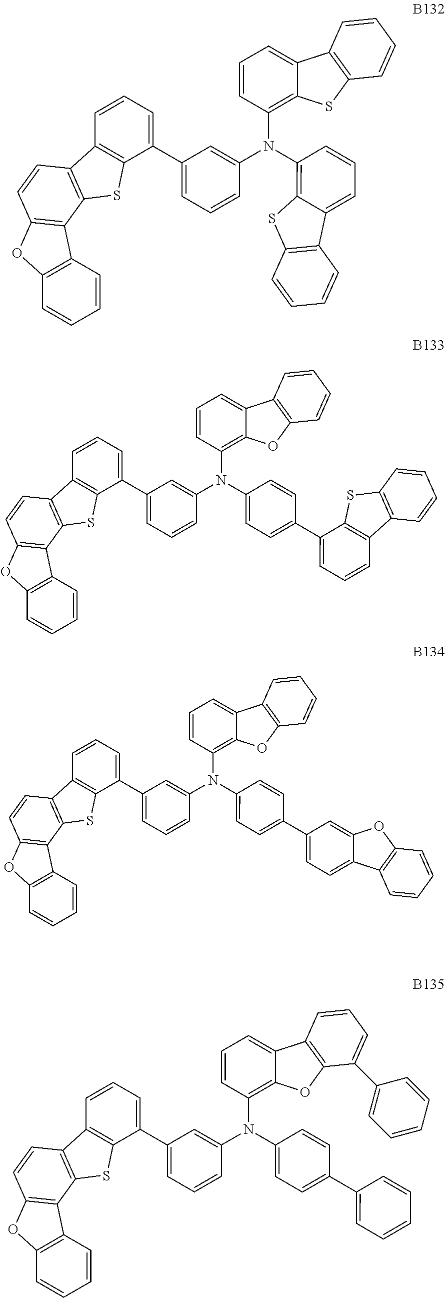 [SMILES: c1cc(-c2cccc3c2sc2c3ccc3oc4ccccc4c32)cc(N(c2cccc3c2sc2ccccc23)c2cccc3c2sc2ccccc23)c1 ; c1cc(-c2cccc3c2sc2c3ccc3oc4ccccc4c32)cc(N(c2ccc(-c3cccc4c3sc3ccccc34)cc2)c2cccc3c2oc2ccccc23)c1 ; c1cc(-c2cccc3c2sc2c3ccc3oc4ccccc4c32)cc(N(c2ccc(-c3ccc4c(c3)oc3ccccc34)cc2)c2cccc3c2oc2ccccc23)c1 ; c1ccc(-c2ccc(N(c3cccc(-c4cccc5c4sc4c5ccc5oc6ccccc6c54)c3)c3cccc4c3oc3c(-c5ccccc5)cccc34)cc2)cc1]

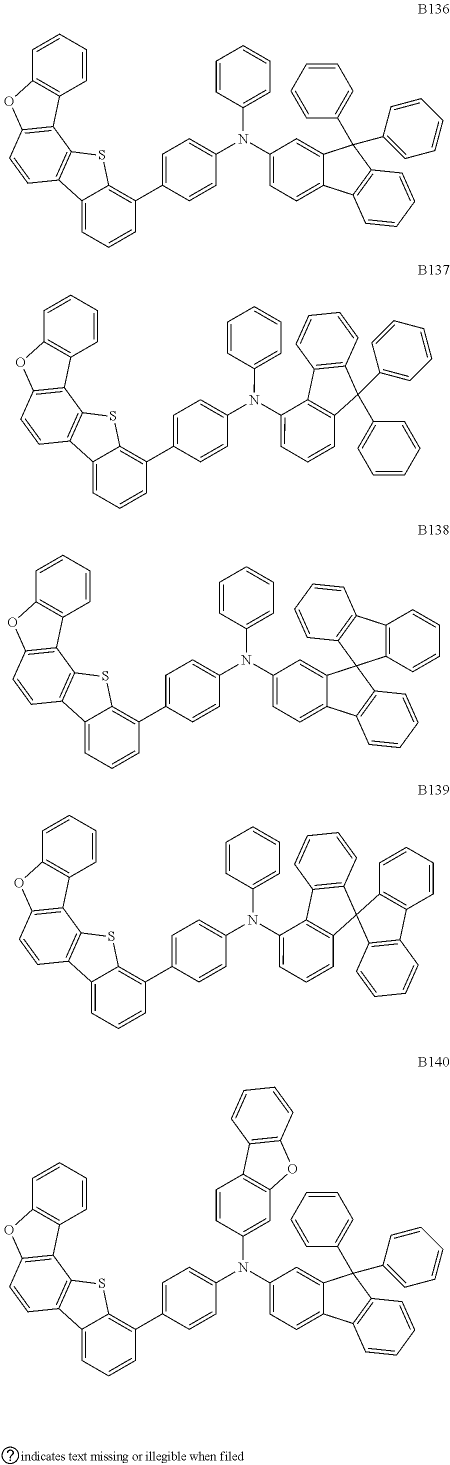 [SMILES: c1ccc(N(c2ccc(-c3cccc4c3sc3c4ccc4oc5ccccc5c43)cc2)c2ccc3c(c2)C(c2ccccc2)(c2ccccc2)c2ccccc2-3)cc1 ; c1ccc(N(c2ccc(-c3cccc4c3sc3c4ccc4oc5ccccc5c43)cc2)c2cccc3c2-c2ccccc2C3(c2ccccc2)c2ccccc2)cc1 ; c1ccc(N(c2ccc(-c3cccc4c3sc3c4ccc4oc5ccccc5c43)cc2)c2ccc3c(c2)C2(c4ccccc4-c4ccccc42)c2ccccc2-3)cc1 ; c1ccc(N(c2ccc(-c3cccc4c3sc3c4ccc4oc5ccccc5c43)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6c5sc5c6ccc6oc7ccccc7c65)cc4)c4ccc5c(c4)oc4ccccc45)cc32)cc1]

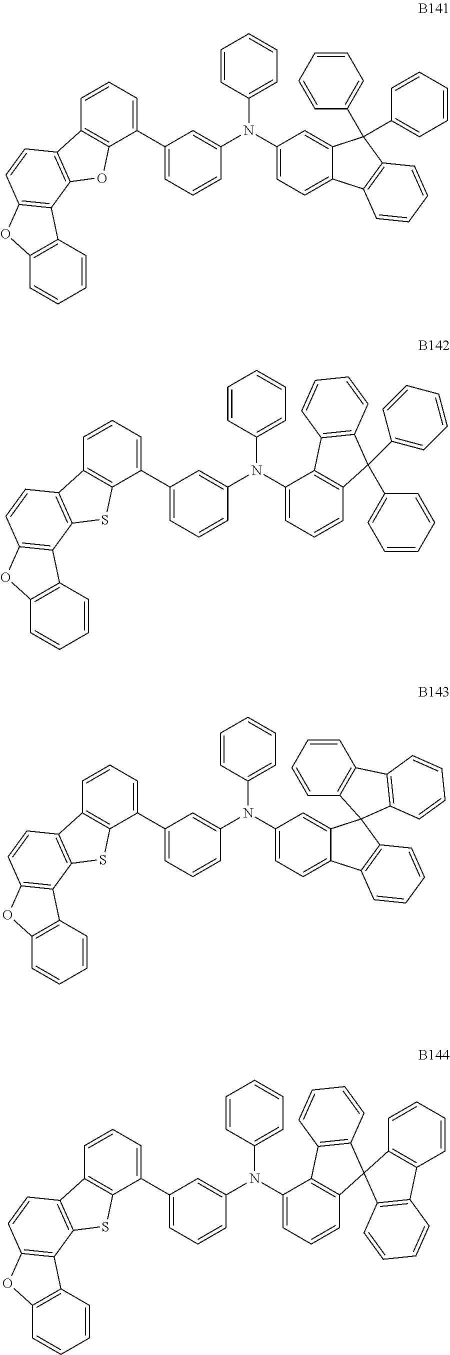 [SMILES: c1ccc(N(c2cccc(-c3cccc4c3oc3c4ccc4oc5ccccc5c43)c2)c2ccc3c(c2)C(c2ccccc2)(c2ccccc2)c2ccccc2-3)cc1 ; c1ccc(N(c2cccc(-c3cccc4c3sc3c4ccc4oc5ccccc5c43)c2)c2cccc3c2-c2ccccc2C3(c2ccccc2)c2ccccc2)cc1 ; c1ccc(N(c2cccc(-c3cccc4c3sc3c4ccc4oc5ccccc5c43)c2)c2ccc3c(c2)C2(c4ccccc4-c4ccccc42)c2ccccc2-3)cc1 ; c1ccc(N(c2cccc(-c3cccc4c3sc3c4ccc4oc5ccccc5c43)c2)c2cccc3c2-c2ccccc2C32c3ccccc3-c3ccccc32)cc1]

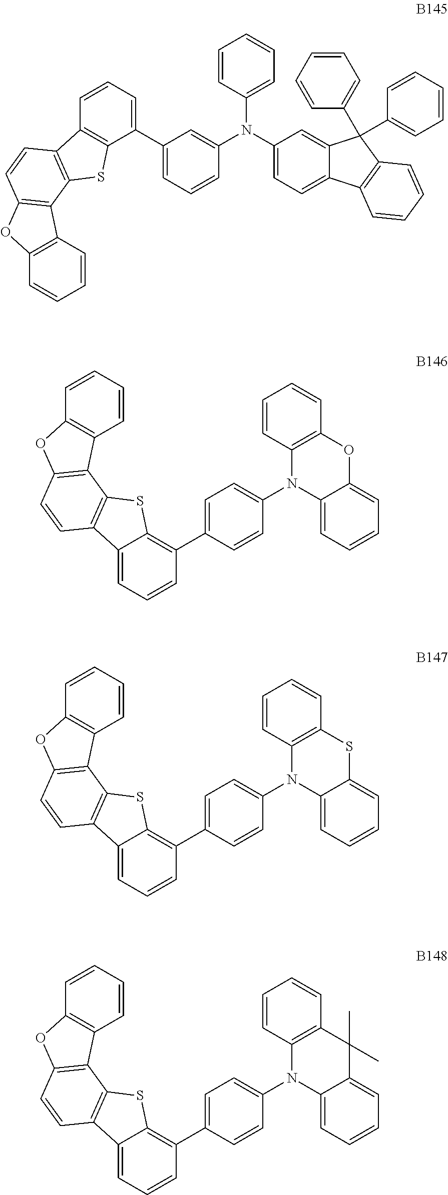 [SMILES: c1ccc(N(c2cccc(-c3cccc4c3sc3c4ccc4oc5ccccc5c43)c2)c2ccc3c(c2)C(c2ccccc2)(c2ccccc2)c2ccccc2-3)cc1 ; c1ccc2c(c1)Oc1ccccc1N2c1ccc(-c2cccc3c2sc2c3ccc3oc4ccccc4c32)cc1 ; c1ccc2c(c1)Sc1ccccc1N2c1ccc(-c2cccc3c2sc2c3ccc3oc4ccccc4c32)cc1 ; CC1(C)c2ccccc2N(c2ccc(-c3cccc4c3sc3c4ccc4oc5ccccc5c43)cc2)c2ccccc21]

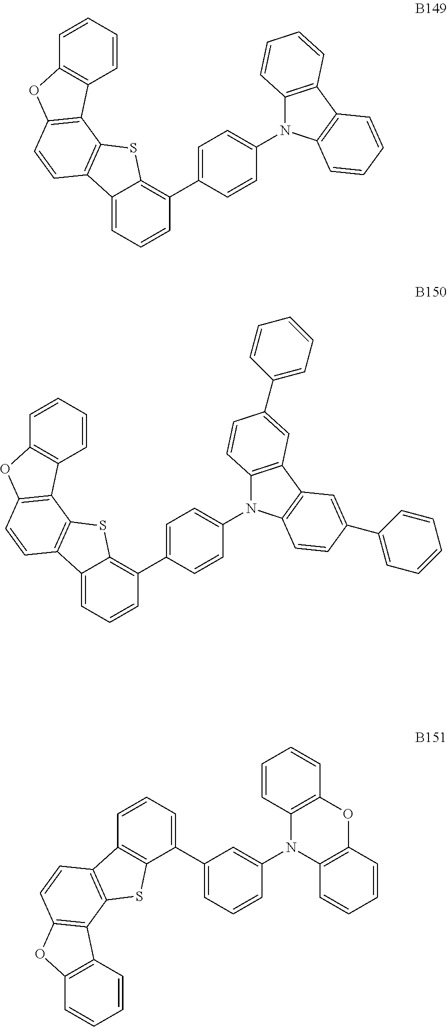 [SMILES: c1ccc2c(c1)oc1ccc3c4cccc(-c5ccc(-n6c7ccccc7c7ccccc76)cc5)c4sc3c12 ; c1ccc(-c2ccc3c(c2)c2cc(-c4ccccc4)ccc2n3-c2ccc(-c3cccc4c3sc3c4ccc4oc5ccccc5c43)cc2)cc1 ; c1cc(-c2cccc3c2sc2c3ccc3oc4ccccc4c32)cc(N2c3ccccc3Oc3ccccc32)c1]

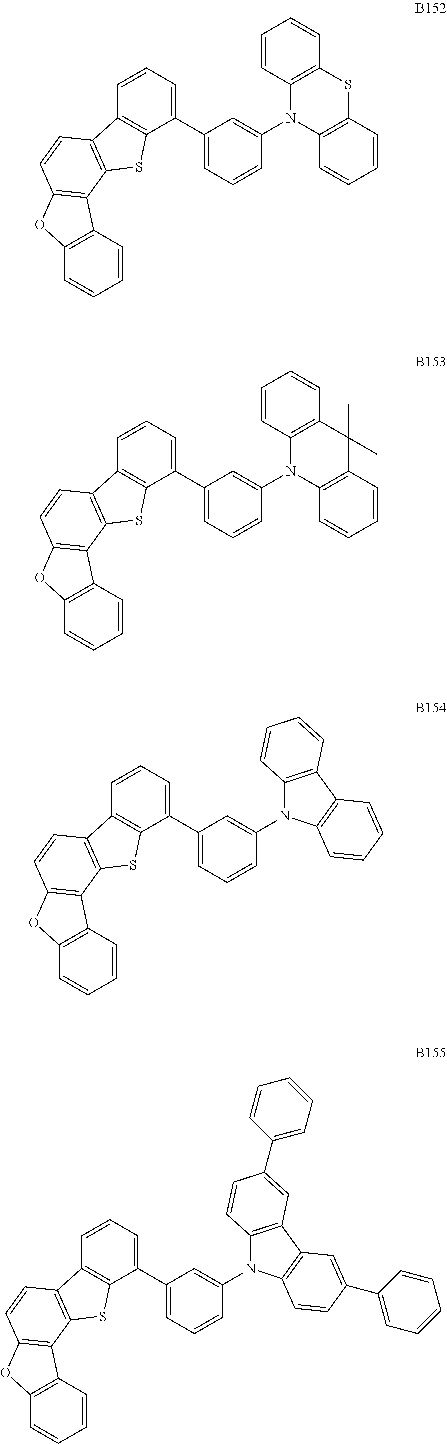 [SMILES: c1cc(-c2cccc3c2sc2c3ccc3oc4ccccc4c32)cc(N2c3ccccc3Sc3ccccc32)c1 ; CC1(C)c2ccccc2N(c2cccc(-c3cccc4c3sc3c4ccc4oc5ccccc5c43)c2)c2ccccc21 ; c1cc(-c2cccc3c2sc2c3ccc3oc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3sc3c4ccc4oc5ccccc5c43)c2)cc1]

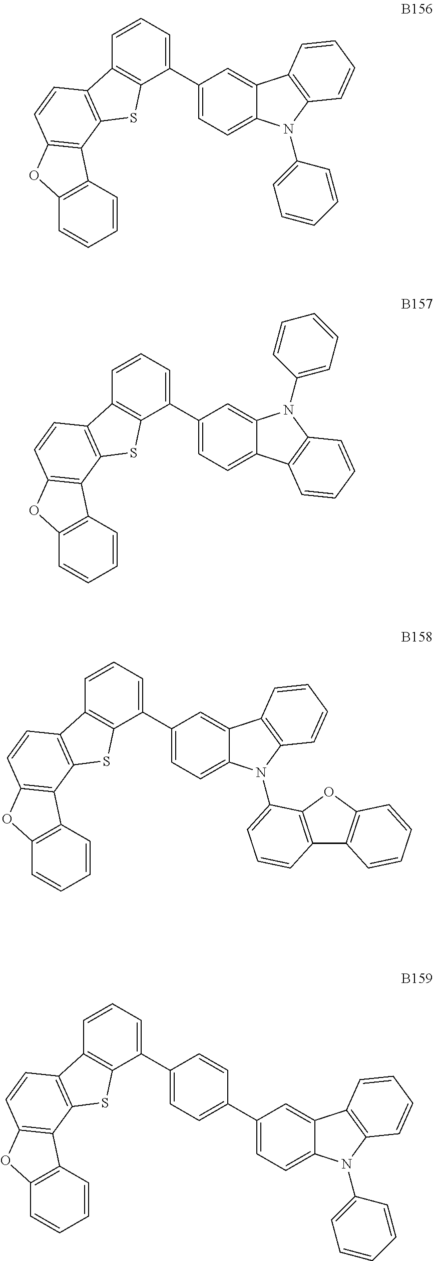 [SMILES: c1ccc(-n2c3ccccc3c3cc(-c4cccc5c4sc4c5ccc5oc6ccccc6c54)ccc32)cc1 ; c1ccc(-n2c3ccccc3c3ccc(-c4cccc5c4sc4c5ccc5oc6ccccc6c54)cc32)cc1 ; c1ccc2c(c1)oc1c(-n3c4ccccc4c4cc(-c5cccc6c5sc5c6ccc6oc7ccccc7c65)ccc43)cccc12 ; c1ccc(-n2c3ccccc3c3cc(-c4ccc(-c5cccc6c5sc5c6ccc6oc7ccccc7c65)cc4)ccc32)cc1]

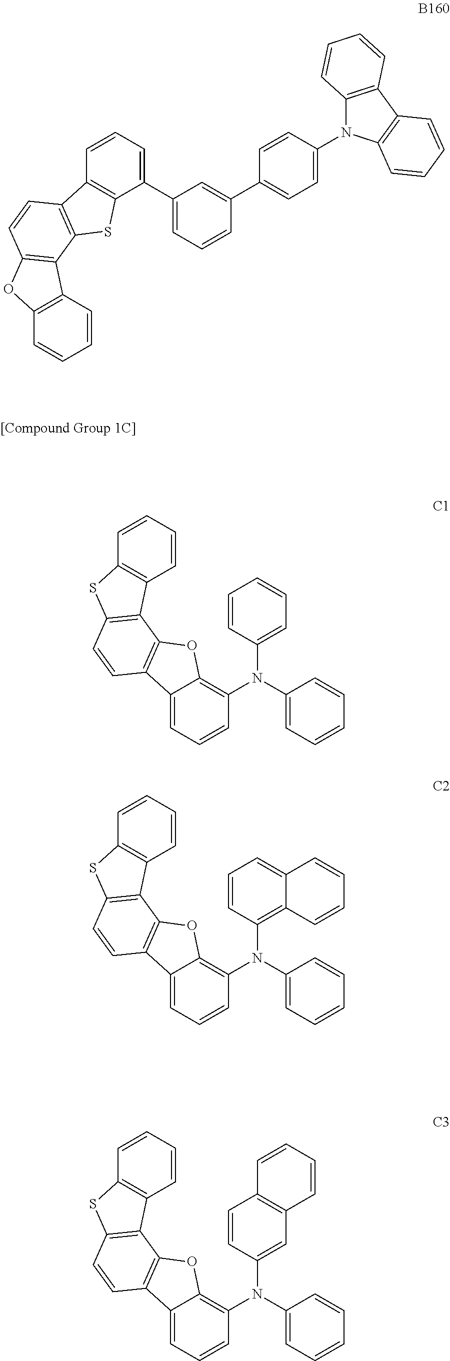 [SMILES: c1cc(-c2ccc(-n3c4ccccc4c4ccccc43)cc2)cc(-c2cccc3c2sc2c3ccc3oc4ccccc4c32)c1 ; c1ccc(N(c2ccccc2)c2cccc3c2oc2c3ccc3sc4ccccc4c32)cc1 ; c1ccc(N(c2cccc3ccccc23)c2cccc3c2oc2c3ccc3sc4ccccc4c32)cc1 ; c1ccc(N(c2ccc3ccccc3c2)c2cccc3c2oc2c3ccc3sc4ccccc4c32)cc1]

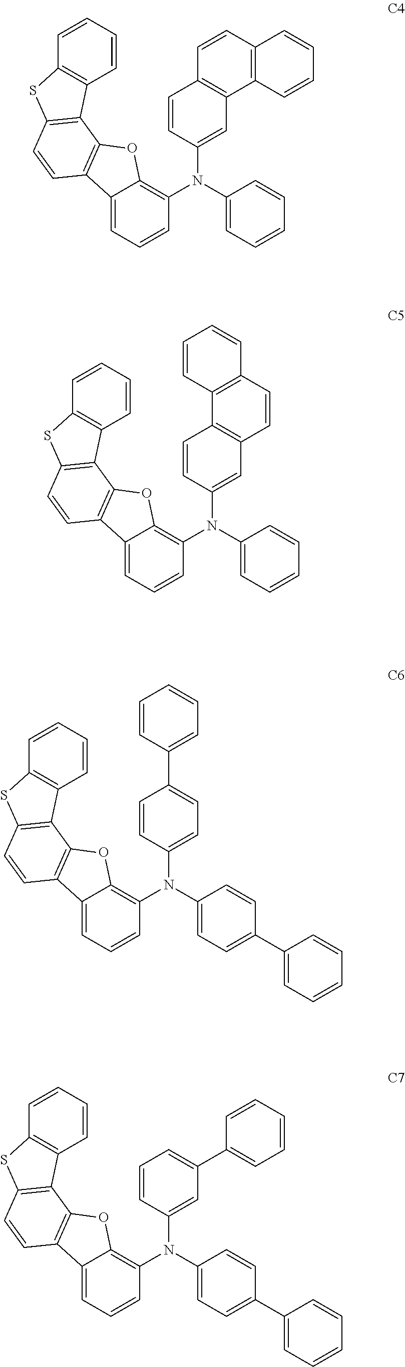 [SMILES: c1ccc(N(c2ccc3ccc4ccccc4c3c2)c2cccc3c2oc2c3ccc3sc4ccccc4c32)cc1 ; c1ccc(N(c2ccc3c(ccc4ccccc43)c2)c2cccc3c2oc2c3ccc3sc4ccccc4c32)cc1 ; c1ccc(-c2ccc(N(c3ccc(-c4ccccc4)cc3)c3cccc4c3oc3c4ccc4sc5ccccc5c43)cc2)cc1 ; c1ccc(-c2ccc(N(c3cccc(-c4ccccc4)c3)c3cccc4c3oc3c4ccc4sc5ccccc5c43)cc2)cc1]

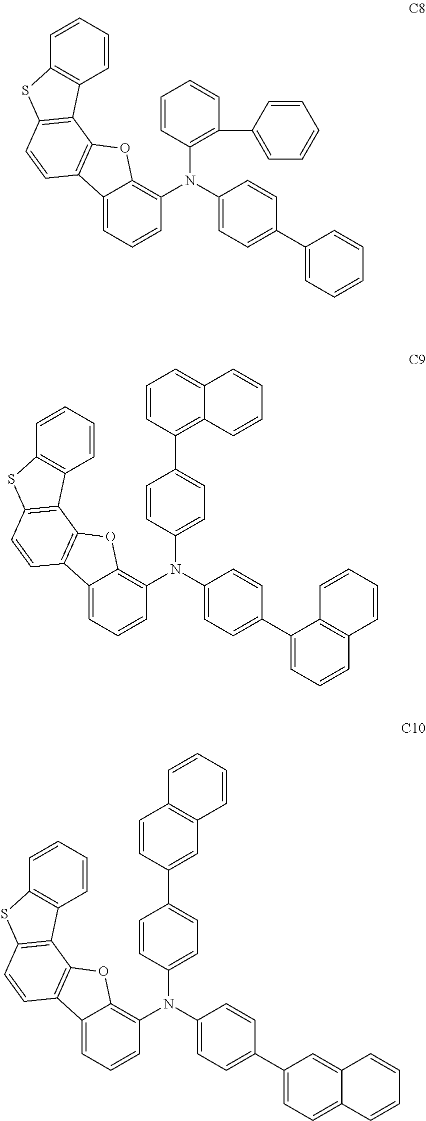 [SMILES: c1ccc(-c2ccc(N(c3ccccc3-c3ccccc3)c3cccc4c3oc3c4ccc4sc5ccccc5c43)cc2)cc1 ; c1ccc2c(-c3ccc(N(c4ccc(-c5cccc6ccccc56)cc4)c4cccc5c4oc4c5ccc5sc6ccccc6c54)cc3)cccc2c1 ; c1ccc2cc(-c3ccc(N(c4ccc(-c5ccc6ccccc6c5)cc4)c4cccc5c4oc4c5ccc5sc6ccccc6c54)cc3)ccc2c1]

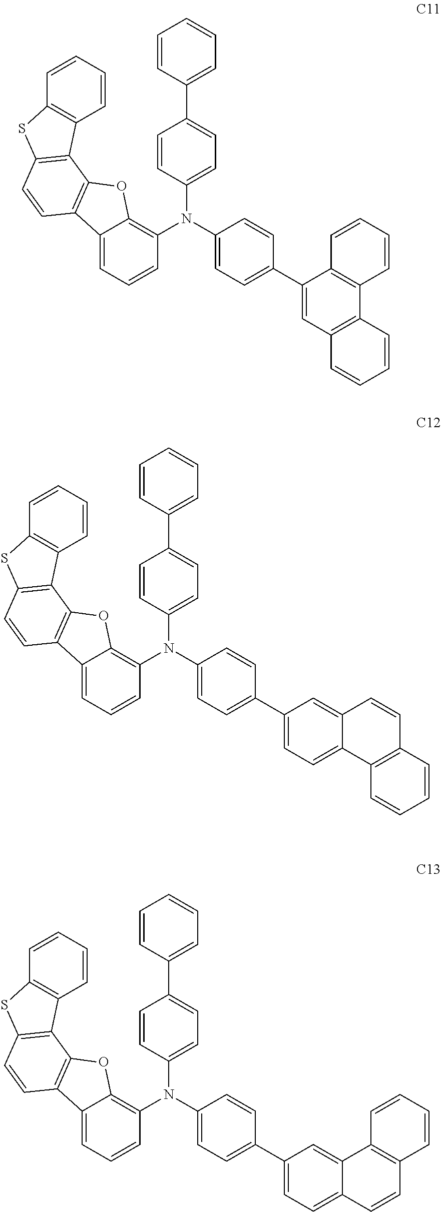 [SMILES: c1ccc(-c2ccc(N(c3ccc(-c4cc5ccccc5c5ccccc45)cc3)c3cccc4c3oc3c4ccc4sc5ccccc5c43)cc2)cc1 ; c1ccc(-c2ccc(N(c3ccc(-c4ccc5c(ccc6ccccc65)c4)cc3)c3cccc4c3oc3c4ccc4sc5ccccc5c43)cc2)cc1 ; c1ccc(-c2ccc(N(c3ccc(-c4ccc5ccc6ccccc6c5c4)cc3)c3cccc4c3oc3c4ccc4sc5ccccc5c43)cc2)cc1]

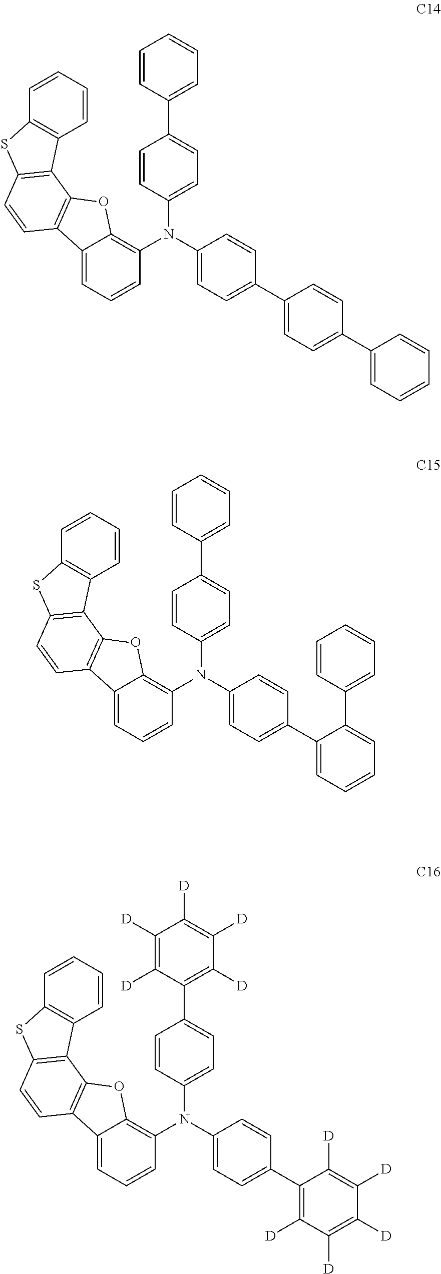 [SMILES: c1ccc(-c2ccc(-c3ccc(N(c4ccc(-c5ccccc5)cc4)c4cccc5c4oc4c5ccc5sc6ccccc6c54)cc3)cc2)cc1 ; c1ccc(-c2ccc(N(c3ccc(-c4ccccc4-c4ccccc4)cc3)c3cccc4c3oc3c4ccc4sc5ccccc5c43)cc2)cc1 ; [2H]c1c([2H])c([2H])c(-c2ccc(N(c3ccc(-c4c([2H])c([2H])c([2H])c([2H])c4[2H])cc3)c3cccc4c3oc3c4ccc4sc5ccccc5c43)cc2)c([2H])c1[2H]]

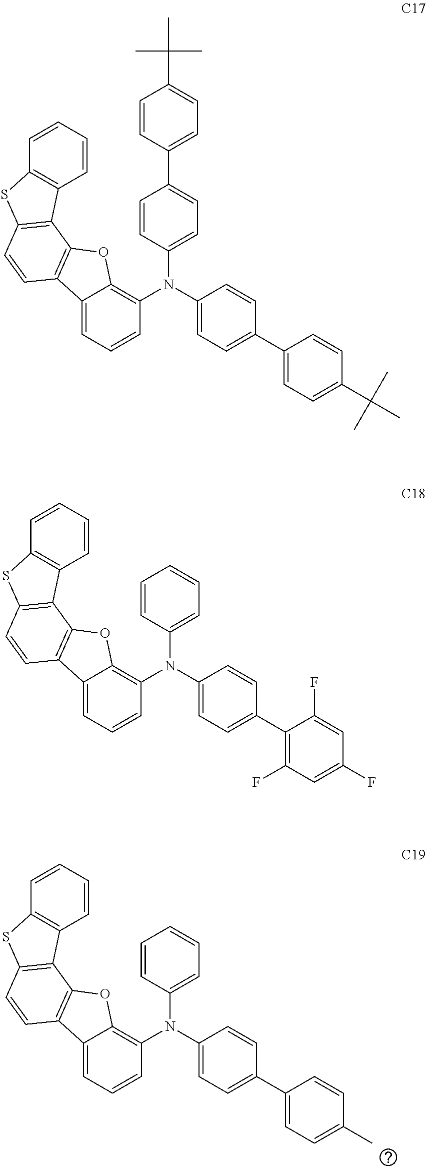 [SMILES: CC(C)(C)c1ccc(-c2ccc(N(c3ccc(-c4ccc(C(C)(C)C)cc4)cc3)c3cccc4c3oc3c4ccc4sc5ccccc5c43)cc2)cc1 ; Fc1cc(F)c(-c2ccc(N(c3ccccc3)c3cccc4c3oc3c4ccc4sc5ccccc5c43)cc2)c(F)c1 ; Cc1ccc(-c2ccc(N(c3ccccc3)c3cccc4c3oc3c4ccc4sc5ccccc5c43)cc2)cc1]

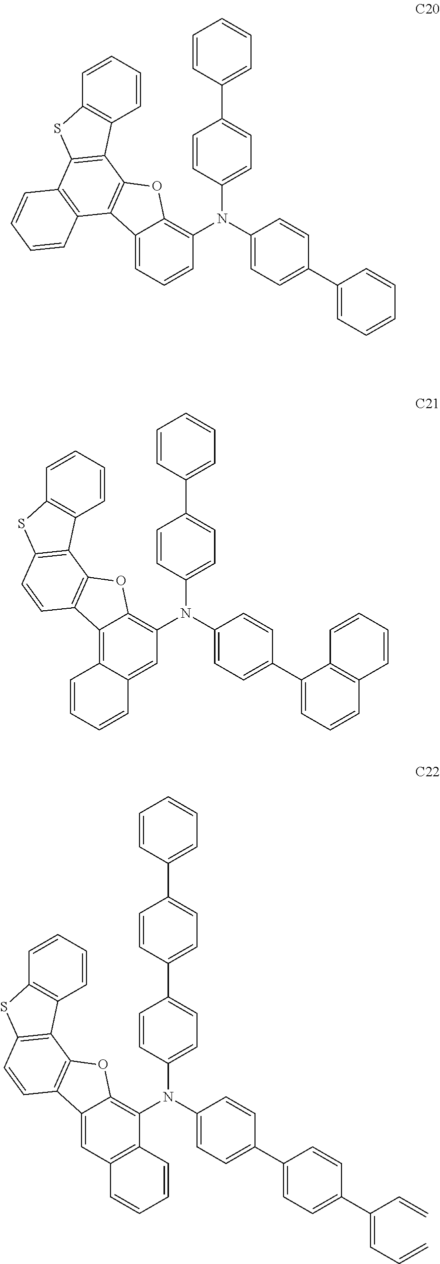 [SMILES: c1ccc(-c2ccc(N(c3ccc(-c4ccccc4)cc3)c3cccc4c3oc3c4c4ccccc4c4sc5ccccc5c34)cc2)cc1 ; c1ccc(-c2ccc(N(c3ccc(-c4cccc5ccccc45)cc3)c3cc4ccccc4c4c3oc3c4ccc4sc5ccccc5c43)cc2)cc1 ; C=C/C=C(\C=C)c1ccc(-c2ccc(N(c3ccc(-c4ccc(-c5ccccc5)cc4)cc3)c3c4ccccc4cc4c3oc3c4ccc4sc5ccccc5c43)cc2)cc1]

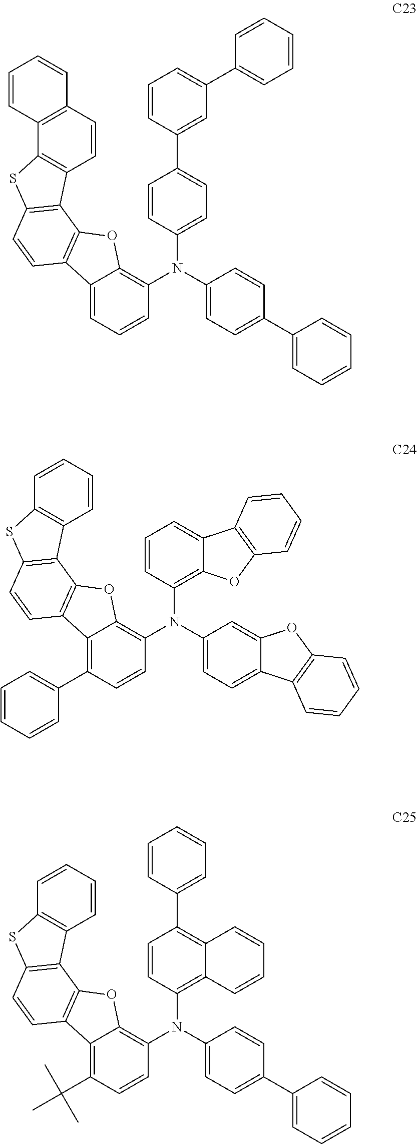 [SMILES: c1ccc(-c2ccc(N(c3ccc(-c4cccc(-c5ccccc5)c4)cc3)c3cccc4c3oc3c4ccc4sc5c6ccccc6ccc5c43)cc2)cc1 ; c1ccc(-c2ccc(N(c3ccc4c(c3)oc3ccccc34)c3cccc4c3oc3ccccc34)c3oc4c(ccc5sc6ccccc6c54)c23)cc1 ; CC(C)(C)c1ccc(N(c2ccc(-c3ccccc3)cc2)c2ccc(-c3ccccc3)c3ccccc23)c2oc3c(ccc4sc5ccccc5c43)c12]

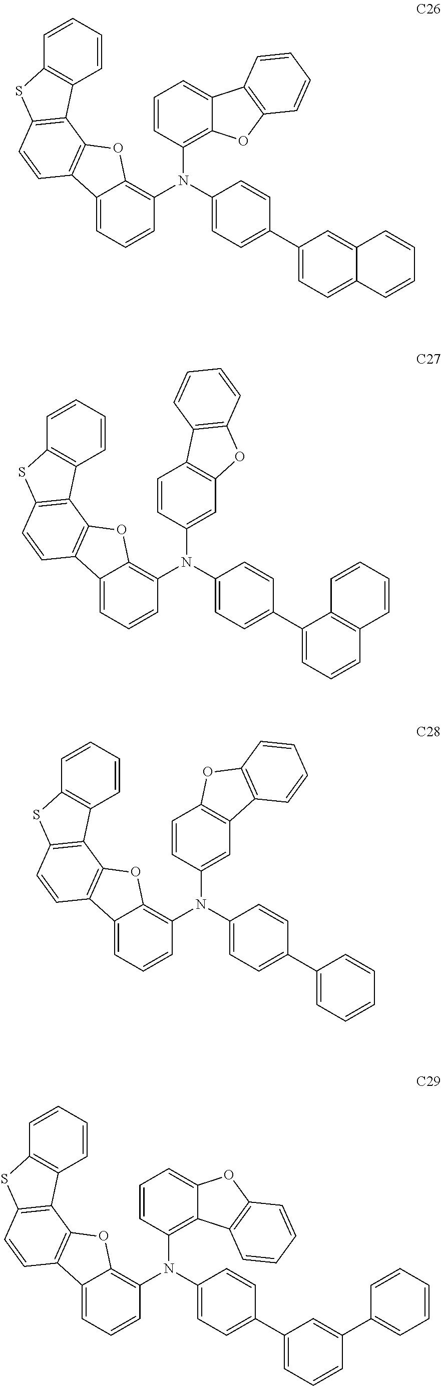 [SMILES: c1ccc2cc(-c3ccc(N(c4cccc5c4oc4ccccc45)c4cccc5c4oc4c5ccc5sc6ccccc6c54)cc3)ccc2c1 ; c1ccc2c(-c3ccc(N(c4ccc5c(c4)oc4ccccc45)c4cccc5c4oc4c5ccc5sc6ccccc6c54)cc3)cccc2c1 ; c1ccc(-c2ccc(N(c3ccc4oc5ccccc5c4c3)c3cccc4c3oc3c4ccc4sc5ccccc5c43)cc2)cc1 ; c1ccc(-c2cccc(-c3ccc(N(c4cccc5c4oc4c5ccc5sc6ccccc6c54)c4cccc5oc6ccccc6c45)cc3)c2)cc1]

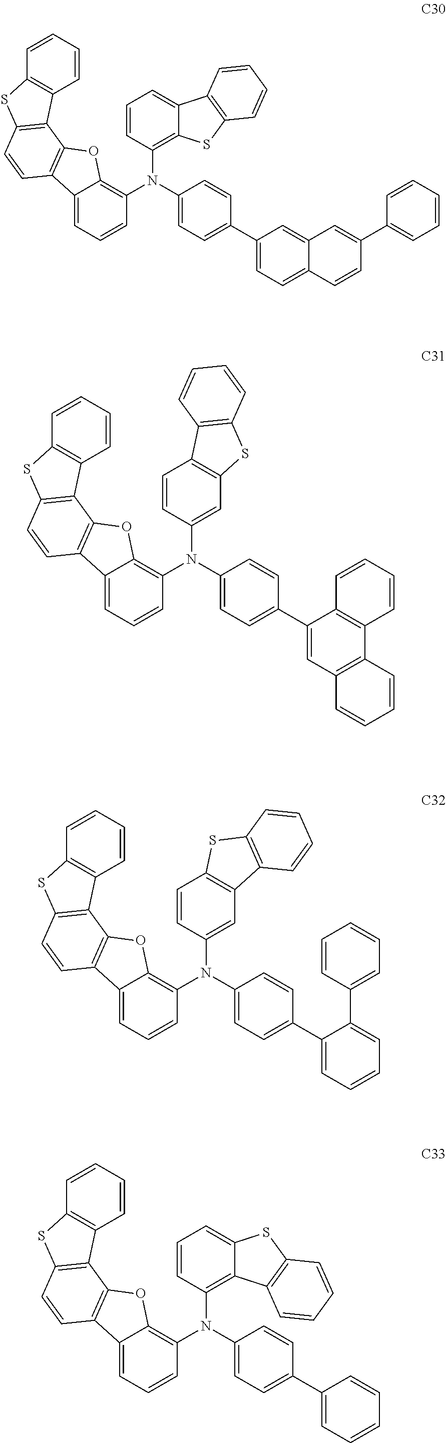 [SMILES: c1ccc(-c2ccc3ccc(-c4ccc(N(c5cccc6c5oc5c6ccc6sc7ccccc7c65)c5cccc6c5sc5ccccc56)cc4)cc3c2)cc1 ; c1ccc2c(c1)cc(-c1ccc(N(c3ccc4c(c3)sc3ccccc34)c3cccc4c3oc3c4ccc4sc5ccccc5c43)cc1)c1ccccc12 ; c1ccc(-c2ccccc2-c2ccc(N(c3ccc4sc5ccccc5c4c3)c3cccc4c3oc3c4ccc4sc5ccccc5c43)cc2)cc1 ; c1ccc(-c2ccc(N(c3cccc4c3oc3c4ccc4sc5ccccc5c43)c3cccc4sc5ccccc5c34)cc2)cc1]

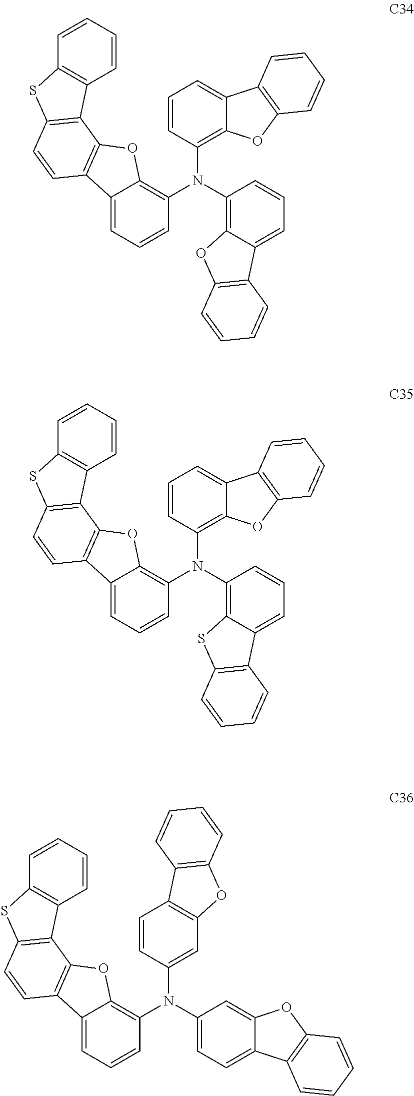 [SMILES: c1ccc2c(c1)oc1c(N(c3cccc4c3oc3ccccc34)c3cccc4c3oc3c4ccc4sc5ccccc5c43)cccc12 ; c1ccc2c(c1)oc1c(N(c3cccc4c3oc3c4ccc4sc5ccccc5c43)c3cccc4c3sc3ccccc34)cccc12 ; c1ccc2c(c1)oc1cc(N(c3ccc4c(c3)oc3ccccc34)c3cccc4c3oc3c4ccc4sc5ccccc5c43)ccc12]

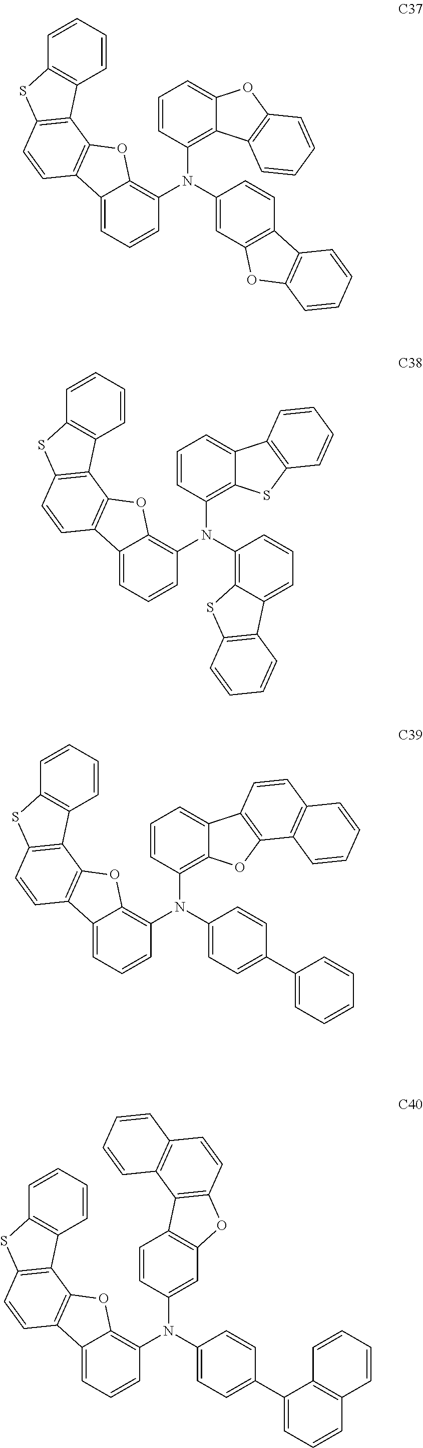 [SMILES: c1ccc2c(c1)oc1cc(N(c3cccc4c3oc3c4ccc4sc5ccccc5c43)c3cccc4oc5ccccc5c34)ccc12 ; c1ccc2c(c1)sc1c(N(c3cccc4c3oc3c4ccc4sc5ccccc5c43)c3cccc4c3sc3ccccc34)cccc12 ; c1ccc(-c2ccc(N(c3cccc4c3oc3c5ccccc5ccc43)c3cccc4c3oc3c4ccc4sc5ccccc5c43)cc2)cc1 ; c1ccc2c(-c3ccc(N(c4ccc5c(c4)oc4ccc6ccccc6c45)c4cccc5c4oc4c5ccc5sc6ccccc6c54)cc3)cccc2c1]

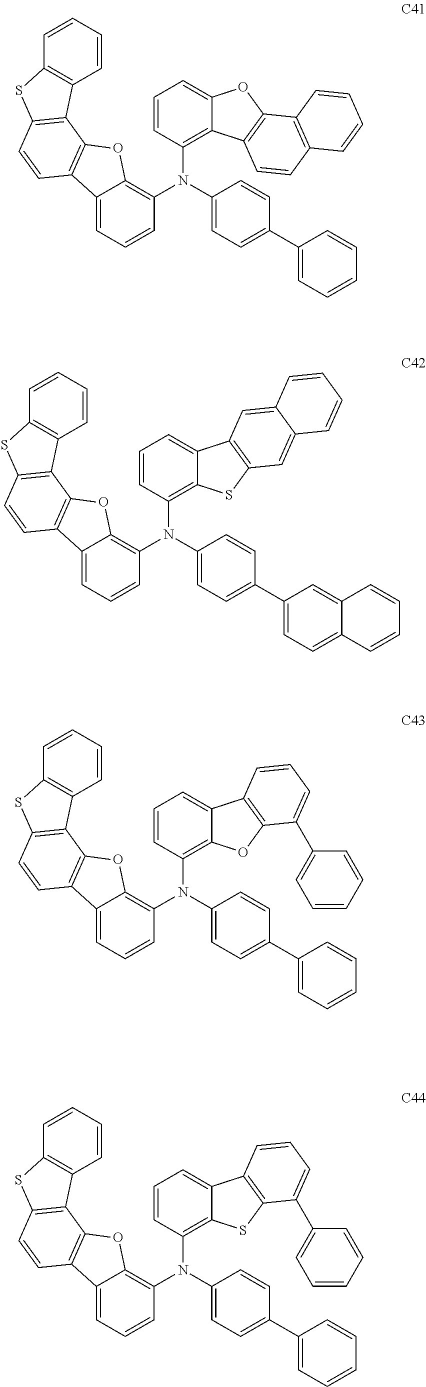 [SMILES: c1ccc(-c2ccc(N(c3cccc4c3oc3c4ccc4sc5ccccc5c43)c3cccc4oc5c6ccccc6ccc5c34)cc2)cc1 ; c1ccc2cc(-c3ccc(N(c4cccc5c4oc4c5ccc5sc6ccccc6c54)c4cccc5c4sc4cc6ccccc6cc45)cc3)ccc2c1 ; c1ccc(-c2ccc(N(c3cccc4c3oc3c(-c5ccccc5)cccc34)c3cccc4c3oc3c4ccc4sc5ccccc5c43)cc2)cc1 ; c1ccc(-c2ccc(N(c3cccc4c3oc3c4ccc4sc5ccccc5c43)c3cccc4c3sc3c(-c5ccccc5)cccc34)cc2)cc1]

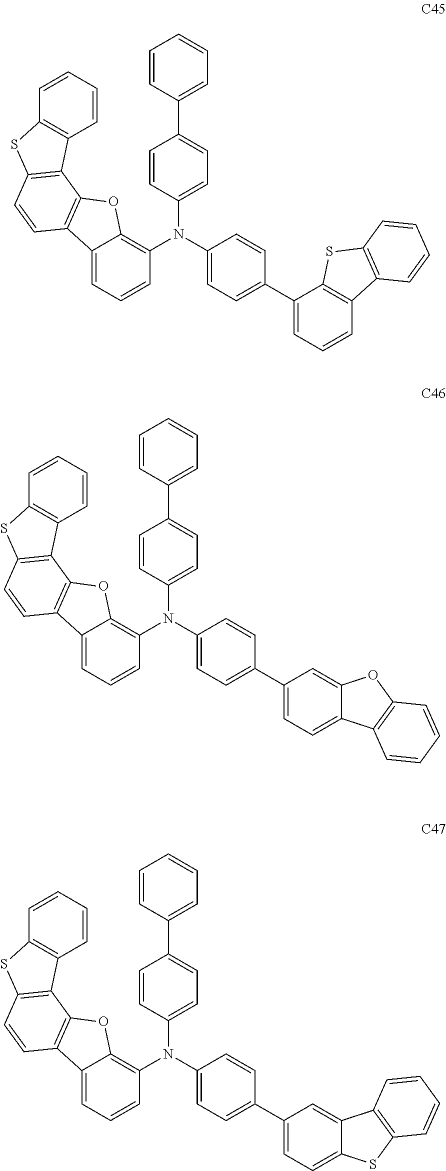 [SMILES: c1ccc(-c2ccc(N(c3ccc(-c4cccc5c4sc4ccccc45)cc3)c3cccc4c3oc3c4ccc4sc5ccccc5c43)cc2)cc1 ; c1ccc(-c2ccc(N(c3ccc(-c4ccc5c(c4)oc4ccccc45)cc3)c3cccc4c3oc3c4ccc4sc5ccccc5c43)cc2)cc1 ; c1ccc(-c2ccc(N(c3ccc(-c4ccc5sc6ccccc6c5c4)cc3)c3cccc4c3oc3c4ccc4sc5ccccc5c43)cc2)cc1]

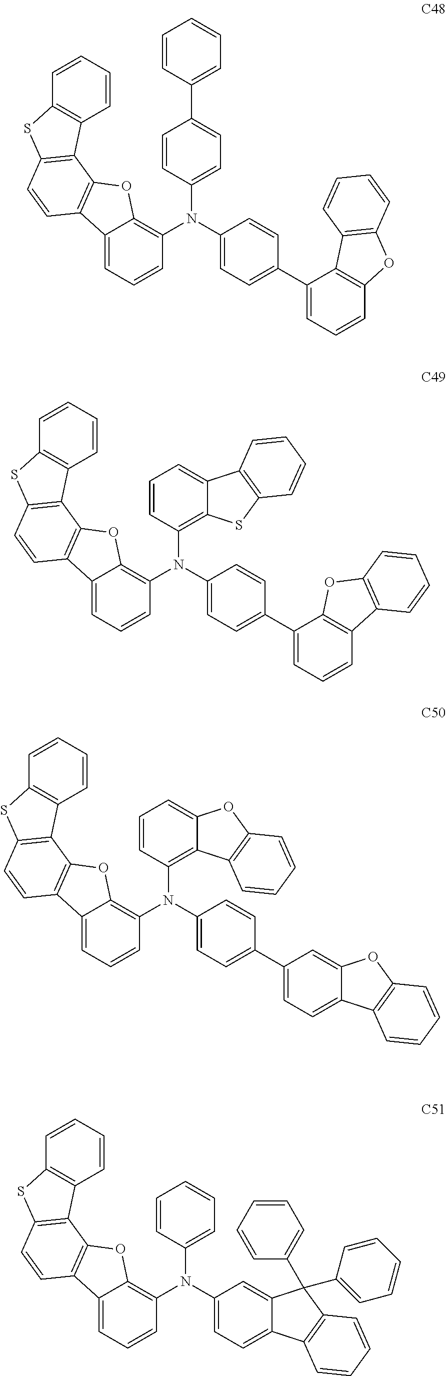 [SMILES: c1ccc(-c2ccc(N(c3ccc(-c4cccc5oc6ccccc6c45)cc3)c3cccc4c3oc3c4ccc4sc5ccccc5c43)cc2)cc1 ; c1ccc2c(c1)oc1c(-c3ccc(N(c4cccc5c4oc4c5ccc5sc6ccccc6c54)c4cccc5c4sc4ccccc45)cc3)cccc12 ; c1ccc2c(c1)oc1cc(-c3ccc(N(c4cccc5c4oc4c5ccc5sc6ccccc6c54)c4cccc5oc6ccccc6c45)cc3)ccc12 ; c1ccc(N(c2ccc3c(c2)C(c2ccccc2)(c2ccccc2)c2ccccc2-3)c2cccc3c2oc2c3ccc3sc4ccccc4c32)cc1]

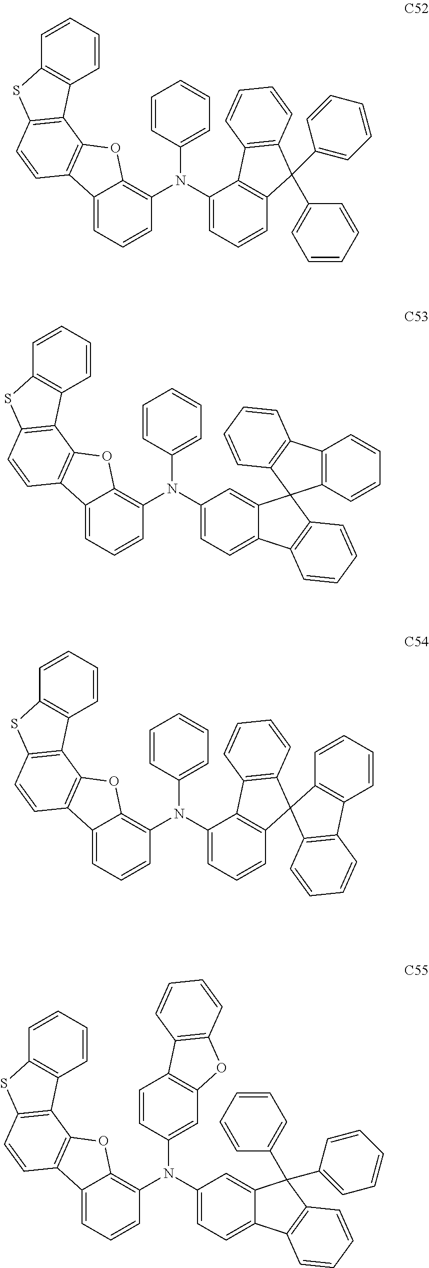 [SMILES: c1ccc(N(c2cccc3c2-c2ccccc2C3(c2ccccc2)c2ccccc2)c2cccc3c2oc2c3ccc3sc4ccccc4c32)cc1 ; c1ccc(N(c2ccc3c(c2)C2(c4ccccc4-c4ccccc42)c2ccccc2-3)c2cccc3c2oc2c3ccc3sc4ccccc4c32)cc1 ; c1ccc(N(c2cccc3c2-c2ccccc2C32c3ccccc3-c3ccccc32)c2cccc3c2oc2c3ccc3sc4ccccc4c32)cc1 ; c1ccc(C2(c3ccccc3)c3ccccc3-c3ccc(N(c4ccc5c(c4)oc4ccccc45)c4cccc5c4oc4c5ccc5sc6ccccc6c54)cc32)cc1]

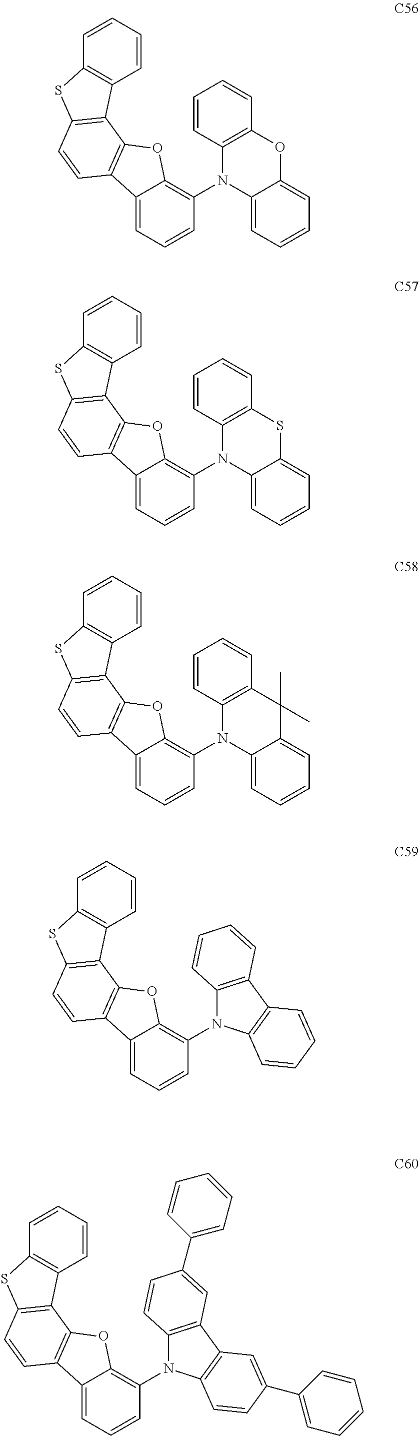 [SMILES: c1ccc2c(c1)Oc1ccccc1N2c1cccc2c1oc1c2ccc2sc3ccccc3c21 ; c1ccc2c(c1)Sc1ccccc1N2c1cccc2c1oc1c2ccc2sc3ccccc3c21 ; CC1(C)c2ccccc2N(c2cccc3c2oc2c3ccc3sc4ccccc4c32)c2ccccc21 ; c1ccc2c(c1)sc1ccc3c4cccc(-n5c6ccccc6c6ccccc65)c4oc3c12 ; c1ccc(-c2ccc3c(c2)c2cc(-c4ccccc4)ccc2n3-c2cccc3c2oc2c3ccc3sc4ccccc4c32)cc1]

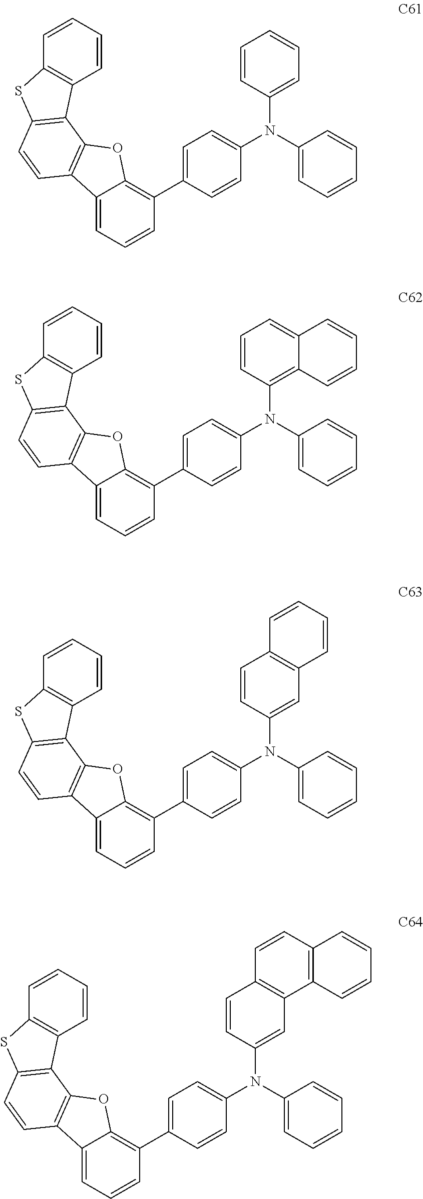 [SMILES: c1ccc(N(c2ccccc2)c2ccc(-c3cccc4c3oc3c4ccc4sc5ccccc5c43)cc2)cc1 ; c1ccc(N(c2ccc(-c3cccc4c3oc3c4ccc4sc5ccccc5c43)cc2)c2cccc3ccccc23)cc1 ; c1ccc(N(c2ccc(-c3cccc4c3oc3c4ccc4sc5ccccc5c43)cc2)c2ccc3ccccc3c2)cc1 ; c1ccc(N(c2ccc(-c3cccc4c3oc3c4ccc4sc5ccccc5c43)cc2)c2ccc3ccc4ccccc4c3c2)cc1]

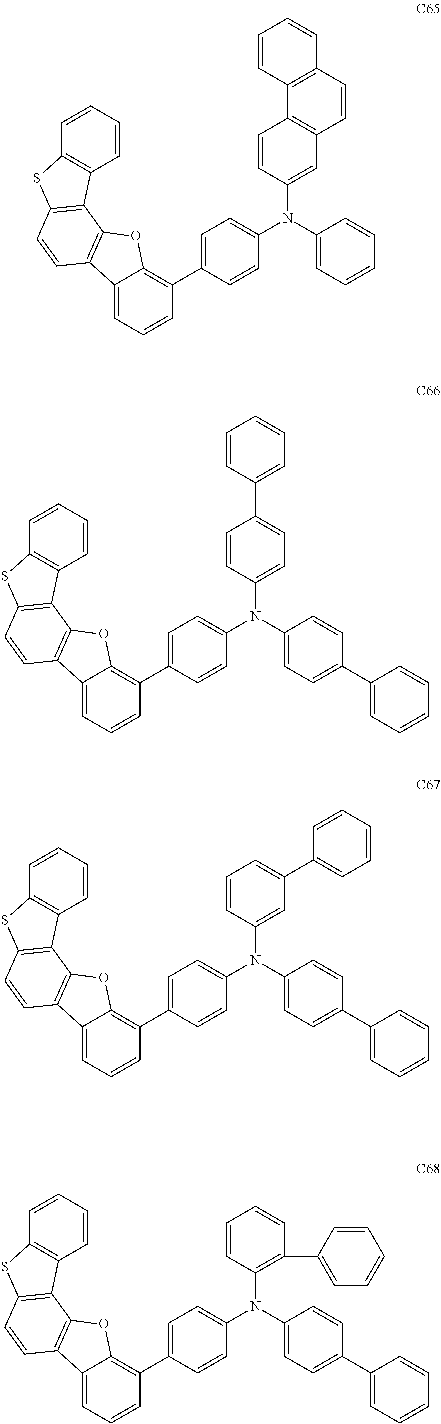 [SMILES: c1ccc(N(c2ccc(-c3cccc4c3oc3c4ccc4sc5ccccc5c43)cc2)c2ccc3c(ccc4ccccc43)c2)cc1 ; c1ccc(-c2ccc(N(c3ccc(-c4ccccc4)cc3)c3ccc(-c4cccc5c4oc4c5ccc5sc6ccccc6c54)cc3)cc2)cc1 ; c1ccc(-c2ccc(N(c3ccc(-c4cccc5c4oc4c5ccc5sc6ccccc6c54)cc3)c3cccc(-c4ccccc4)c3)cc2)cc1 ; c1ccc(-c2ccc(N(c3ccc(-c4cccc5c4oc4c5ccc5sc6ccccc6c54)cc3)c3ccccc3-c3ccccc3)cc2)cc1]

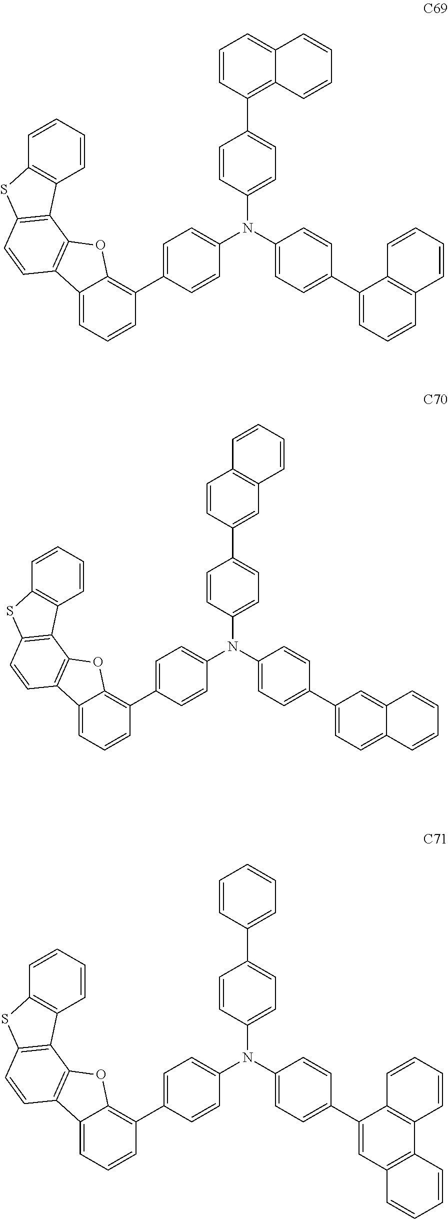 [SMILES: c1ccc2c(-c3ccc(N(c4ccc(-c5cccc6ccccc56)cc4)c4ccc(-c5cccc6c5oc5c6ccc6sc7ccccc7c65)cc4)cc3)cccc2c1 ; c1ccc2cc(-c3ccc(N(c4ccc(-c5ccc6ccccc6c5)cc4)c4ccc(-c5cccc6c5oc5c6ccc6sc7ccccc7c65)cc4)cc3)ccc2c1 ; c1ccc(-c2ccc(N(c3ccc(-c4cc5ccccc5c5ccccc45)cc3)c3ccc(-c4cccc5c4oc4c5ccc5sc6ccccc6c54)cc3)cc2)cc1]

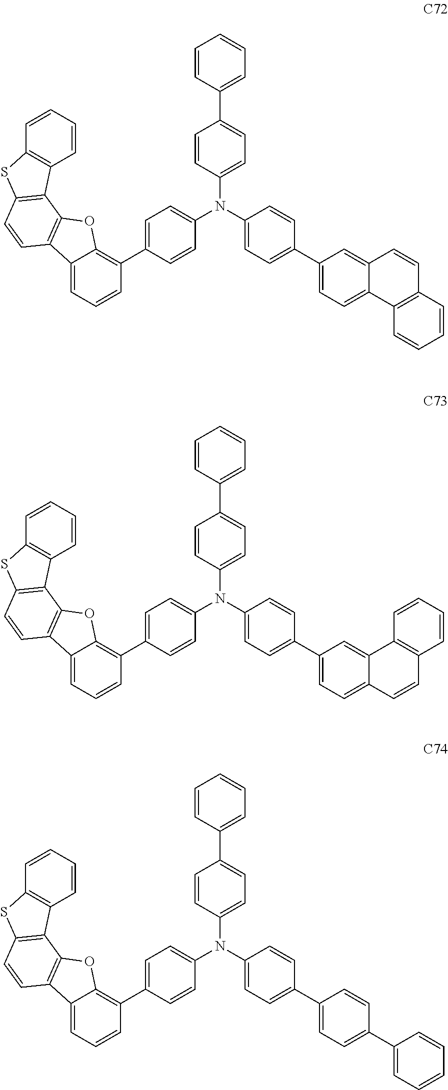 [SMILES: c1ccc(-c2ccc(N(c3ccc(-c4ccc5c(ccc6ccccc65)c4)cc3)c3ccc(-c4cccc5c4oc4c5ccc5sc6ccccc6c54)cc3)cc2)cc1 ; c1ccc(-c2ccc(N(c3ccc(-c4ccc5ccc6ccccc6c5c4)cc3)c3ccc(-c4cccc5c4oc4c5ccc5sc6ccccc6c54)cc3)cc2)cc1 ; c1ccc(-c2ccc(-c3ccc(N(c4ccc(-c5ccccc5)cc4)c4ccc(-c5cccc6c5oc5c6ccc6sc7ccccc7c65)cc4)cc3)cc2)cc1]

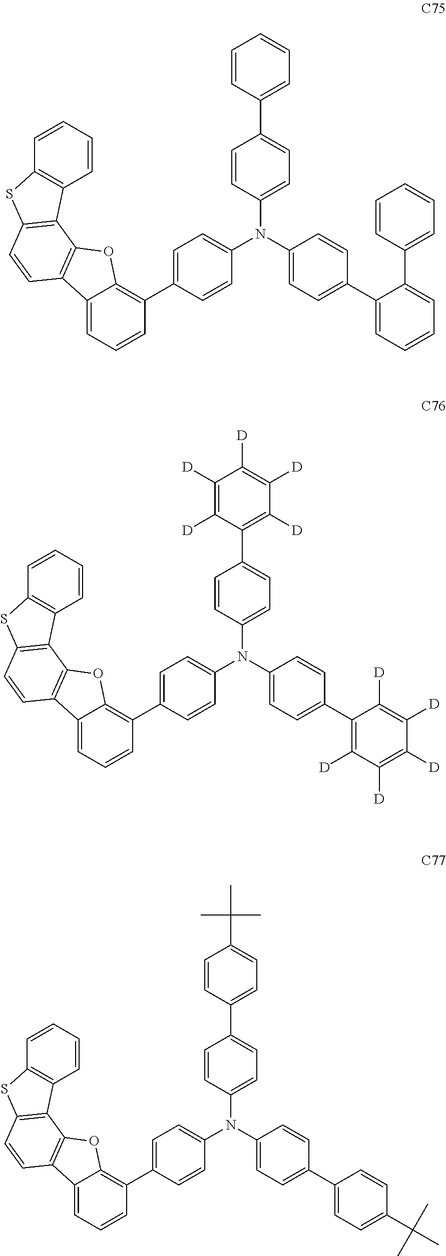 [SMILES: c1ccc(-c2ccc(N(c3ccc(-c4ccccc4-c4ccccc4)cc3)c3ccc(-c4cccc5c4oc4c5ccc5sc6ccccc6c54)cc3)cc2)cc1 ; [2H]c1c([2H])c([2H])c(-c2ccc(N(c3ccc(-c4c([2H])c([2H])c([2H])c([2H])c4[2H])cc3)c3ccc(-c4cccc5c4oc4c5ccc5sc6ccccc6c54)cc3)cc2)c([2H])c1[2H] ; CC(C)(C)c1ccc(-c2ccc(N(c3ccc(-c4ccc(C(C)(C)C)cc4)cc3)c3ccc(-c4cccc5c4oc4c5ccc5sc6ccccc6c54)cc3)cc2)cc1]

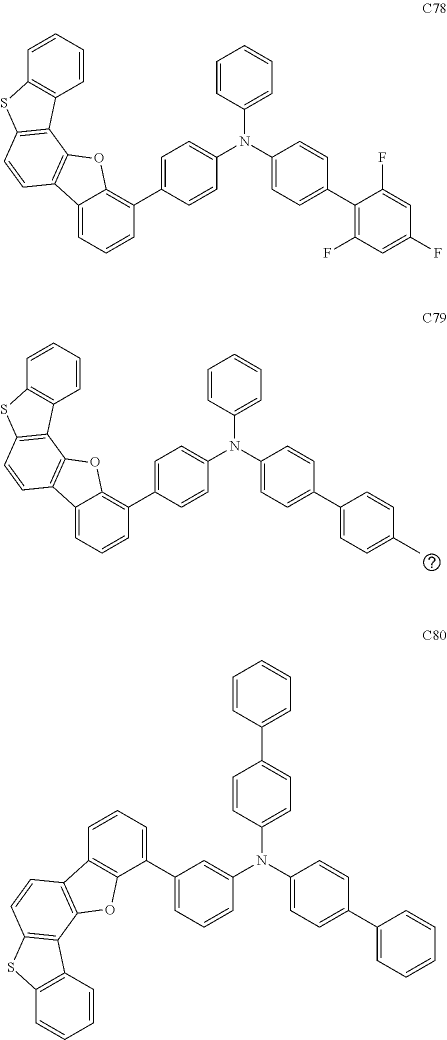 [SMILES: Fc1cc(F)c(-c2ccc(N(c3ccccc3)c3ccc(-c4cccc5c4oc4c5ccc5sc6ccccc6c54)cc3)cc2)c(F)c1 ; Cc1ccc(-c2ccc(N(c3ccccc3)c3ccc(-c4cccc5c4oc4c5ccc5sc6ccccc6c54)cc3)cc2)cc1 ; c1ccc(-c2ccc(N(c3ccc(-c4ccccc4)cc3)c3cccc(-c4cccc5c4oc4c5ccc5sc6ccccc6c54)c3)cc2)cc1]

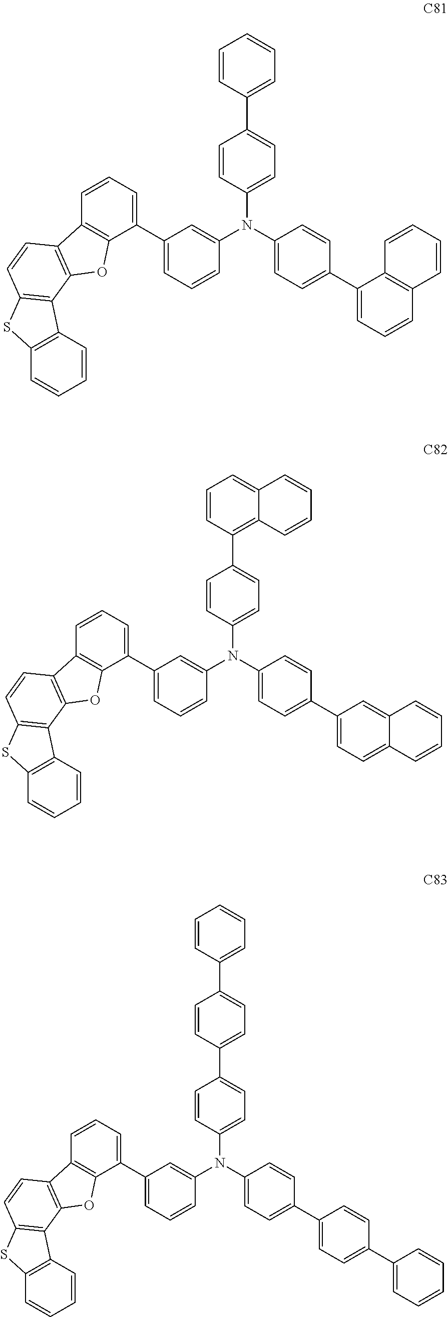 [SMILES: c1ccc(-c2ccc(N(c3ccc(-c4cccc5ccccc45)cc3)c3cccc(-c4cccc5c4oc4c5ccc5sc6ccccc6c54)c3)cc2)cc1 ; c1cc(-c2cccc3c2oc2c3ccc3sc4ccccc4c32)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6c5oc5c6ccc6sc7ccccc7c65)c4)cc3)cc2)cc1]

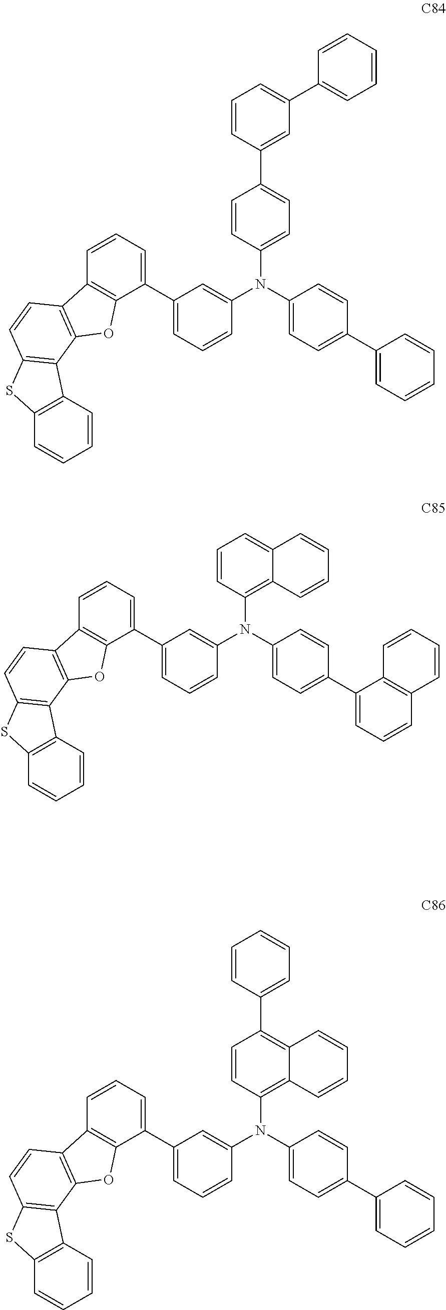 [SMILES: c1ccc(-c2ccc(N(c3ccc(-c4cccc(-c5ccccc5)c4)cc3)c3cccc(-c4cccc5c4oc4c5ccc5sc6ccccc6c54)c3)cc2)cc1 ; c1cc(-c2cccc3c2oc2c3ccc3sc4ccccc4c32)cc(N(c2ccc(-c3cccc4ccccc34)cc2)c2cccc3ccccc23)c1 ; c1ccc(-c2ccc(N(c3cccc(-c4cccc5c4oc4c5ccc5sc6ccccc6c54)c3)c3ccc(-c4ccccc4)c4ccccc34)cc2)cc1]

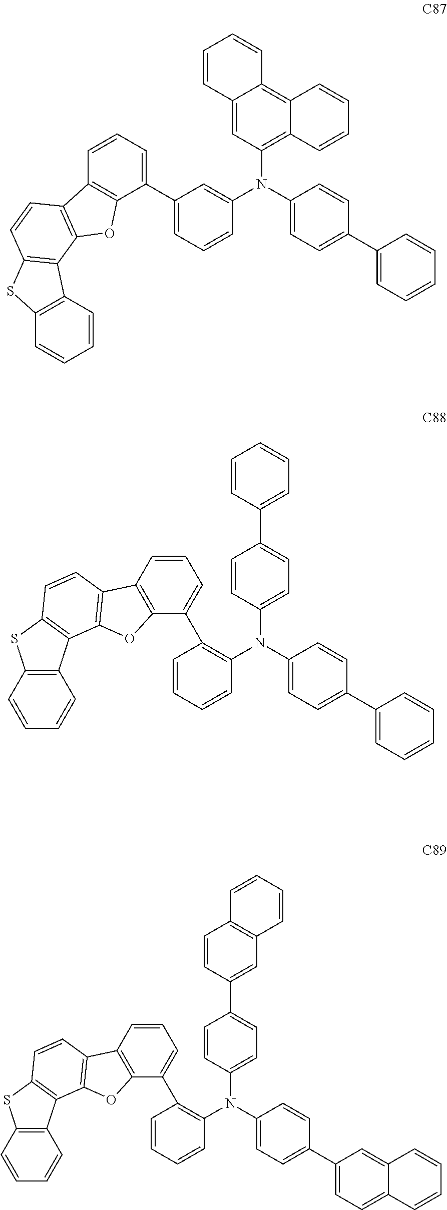 [SMILES: c1ccc(-c2ccc(N(c3cccc(-c4cccc5c4oc4c5ccc5sc6ccccc6c54)c3)c3cc4ccccc4c4ccccc34)cc2)cc1 ; c1ccc(-c2ccc(N(c3ccc(-c4ccccc4)cc3)c3ccccc3-c3cccc4c3oc3c4ccc4sc5ccccc5c43)cc2)cc1 ; c1ccc(N(c2ccc(-c3ccc4ccccc4c3)cc2)c2ccc(-c3ccc4ccccc4c3)cc2)c(-c2cccc3c2oc2c3ccc3sc4ccccc4c32)c1]

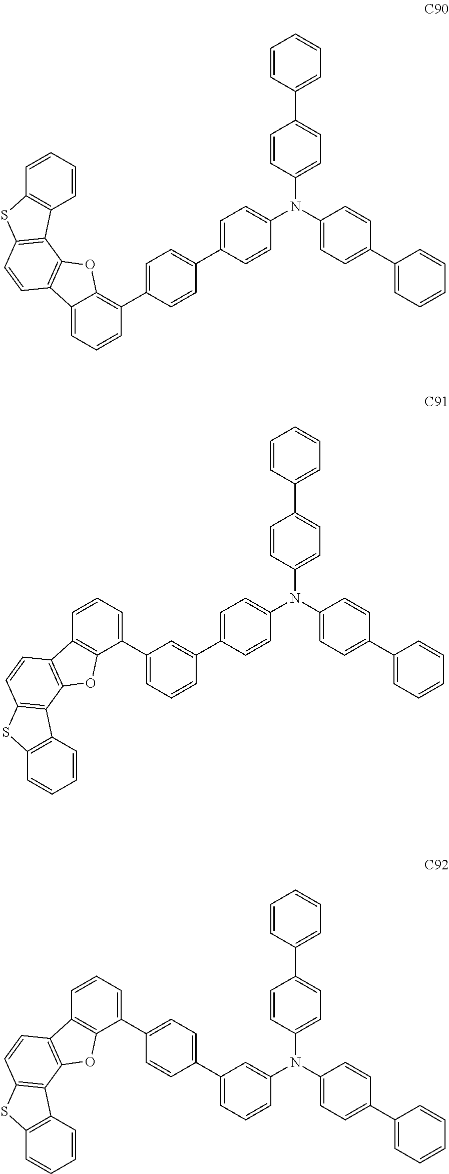 [SMILES: c1ccc(-c2ccc(N(c3ccc(-c4ccccc4)cc3)c3ccc(-c4ccc(-c5cccc6c5oc5c6ccc6sc7ccccc7c65)cc4)cc3)cc2)cc1 ; c1ccc(-c2ccc(N(c3ccc(-c4ccccc4)cc3)c3ccc(-c4cccc(-c5cccc6c5oc5c6ccc6sc7ccccc7c65)c4)cc3)cc2)cc1 ; c1ccc(-c2ccc(N(c3ccc(-c4ccccc4)cc3)c3cccc(-c4ccc(-c5cccc6c5oc5c6ccc6sc7ccccc7c65)cc4)c3)cc2)cc1]

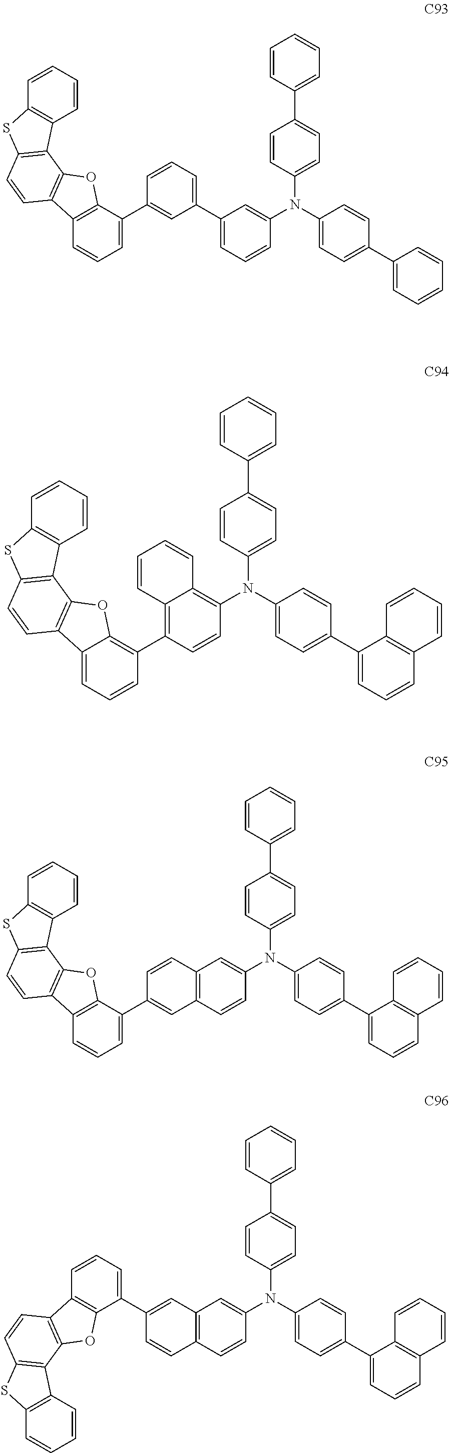 [SMILES: c1ccc(-c2ccc(N(c3ccc(-c4ccccc4)cc3)c3cccc(-c4cccc(-c5cccc6c5oc5c6ccc6sc7ccccc7c65)c4)c3)cc2)cc1 ; c1ccc(-c2ccc(N(c3ccc(-c4cccc5ccccc45)cc3)c3ccc(-c4cccc5c4oc4c5ccc5sc6ccccc6c54)c4ccccc34)cc2)cc1 ; c1ccc(-c2ccc(N(c3ccc(-c4cccc5ccccc45)cc3)c3ccc4cc(-c5cccc6c5oc5c6ccc6sc7ccccc7c65)ccc4c3)cc2)cc1 ; c1ccc(-c2ccc(N(c3ccc(-c4cccc5ccccc45)cc3)c3ccc4ccc(-c5cccc6c5oc5c6ccc6sc7ccccc7c65)cc4c3)cc2)cc1]

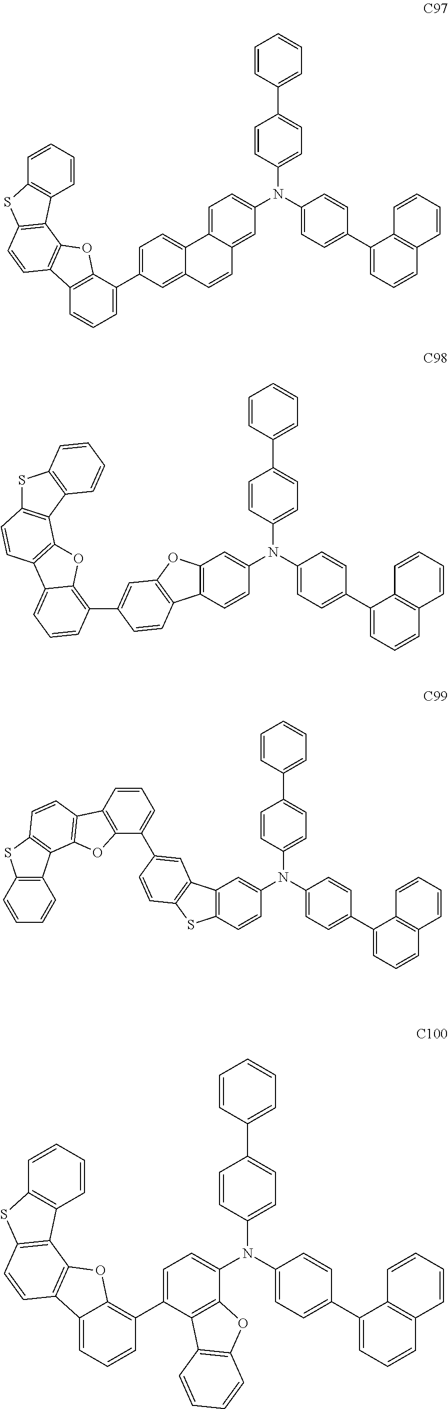 [SMILES: c1ccc(-c2ccc(N(c3ccc(-c4cccc5ccccc45)cc3)c3ccc4c(ccc5cc(-c6cccc7c6oc6c7ccc7sc8ccccc8c76)ccc54)c3)cc2)cc1 ; c1ccc(-c2ccc(N(c3ccc(-c4cccc5ccccc45)cc3)c3ccc4c(c3)oc3cc(-c5cccc6c5oc5c6ccc6sc7ccccc7c65)ccc34)cc2)cc1 ; c1ccc(-c2ccc(N(c3ccc(-c4cccc5ccccc45)cc3)c3ccc4sc5ccc(-c6cccc7c6oc6c7ccc7sc8ccccc8c76)cc5c4c3)cc2)cc1 ; c1ccc(-c2ccc(N(c3ccc(-c4cccc5ccccc45)cc3)c3ccc(-c4cccc5c4oc4c5ccc5sc6ccccc6c54)c4c3oc3ccccc34)cc2)cc1]

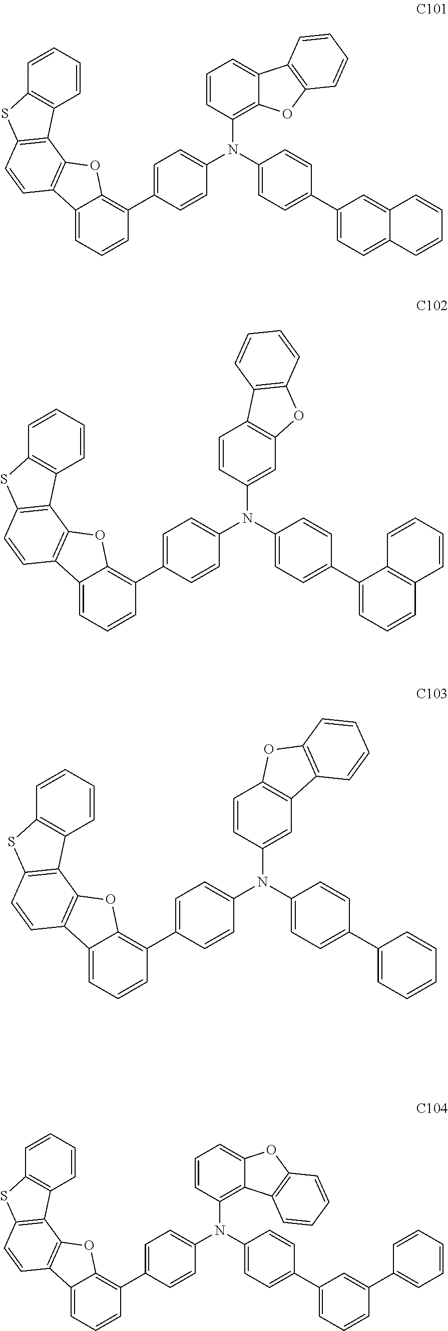 [SMILES: c1ccc2cc(-c3ccc(N(c4ccc(-c5cccc6c5oc5c6ccc6sc7ccccc7c65)cc4)c4cccc5c4oc4ccccc45)cc3)ccc2c1 ; c1ccc2c(-c3ccc(N(c4ccc(-c5cccc6c5oc5c6ccc6sc7ccccc7c65)cc4)c4ccc5c(c4)oc4ccccc45)cc3)cccc2c1 ; c1ccc(-c2ccc(N(c3ccc(-c4cccc5c4oc4c5ccc5sc6ccccc6c54)cc3)c3ccc4oc5ccccc5c4c3)cc2)cc1 ; c1ccc(-c2cccc(-c3ccc(N(c4ccc(-c5cccc6c5oc5c6ccc6sc7ccccc7c65)cc4)c4cccc5oc6ccccc6c45)cc3)c2)cc1]

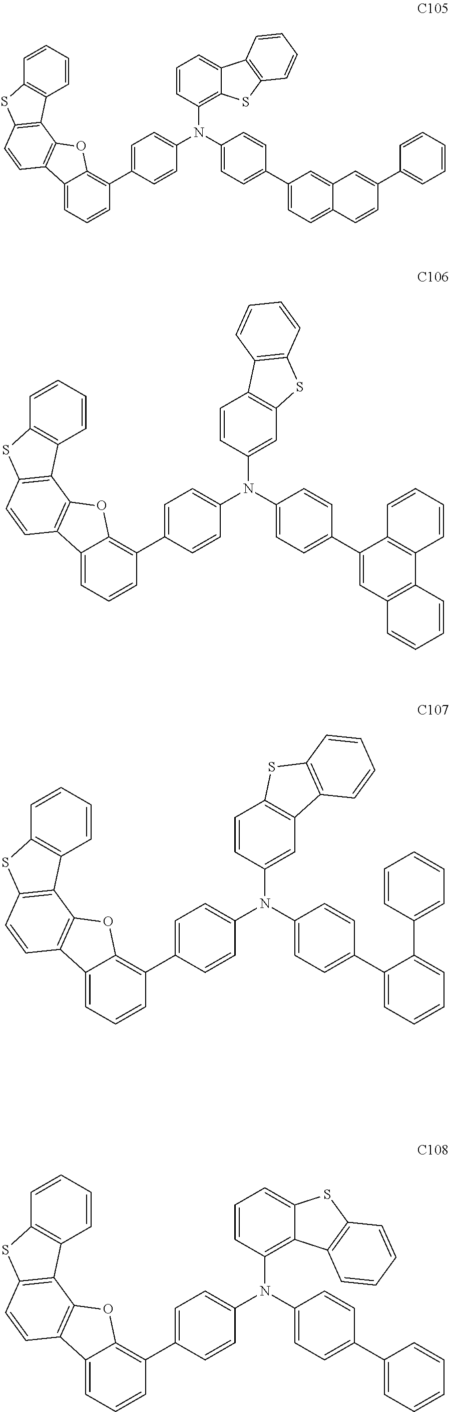 [SMILES: c1ccc(-c2ccc3ccc(-c4ccc(N(c5ccc(-c6cccc7c6oc6c7ccc7sc8ccccc8c76)cc5)c5cccc6c5sc5ccccc56)cc4)cc3c2)cc1 ; c1ccc2c(c1)cc(-c1ccc(N(c3ccc(-c4cccc5c4oc4c5ccc5sc6ccccc6c54)cc3)c3ccc4c(c3)sc3ccccc34)cc1)c1ccccc12 ; c1ccc(-c2ccccc2-c2ccc(N(c3ccc(-c4cccc5c4oc4c5ccc5sc6ccccc6c54)cc3)c3ccc4sc5ccccc5c4c3)cc2)cc1 ; c1ccc(-c2ccc(N(c3ccc(-c4cccc5c4oc4c5ccc5sc6ccccc6c54)cc3)c3cccc4sc5ccccc5c34)cc2)cc1]

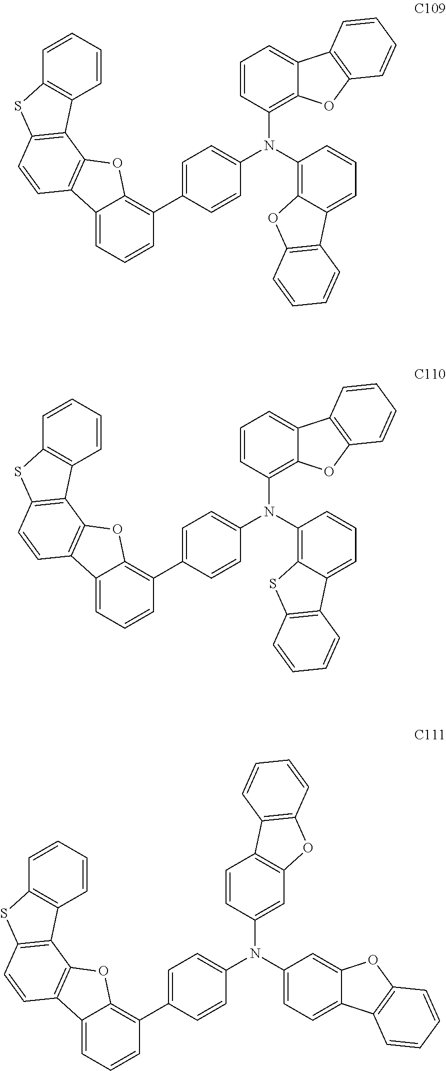 [SMILES: c1ccc2c(c1)oc1c(N(c3ccc(-c4cccc5c4oc4c5ccc5sc6ccccc6c54)cc3)c3cccc4c3oc3ccccc34)cccc12 ; c1ccc2c(c1)oc1c(N(c3ccc(-c4cccc5c4oc4c5ccc5sc6ccccc6c54)cc3)c3cccc4c3sc3ccccc34)cccc12 ; c1ccc2c(c1)oc1cc(N(c3ccc(-c4cccc5c4oc4c5ccc5sc6ccccc6c54)cc3)c3ccc4c(c3)oc3ccccc34)ccc12]

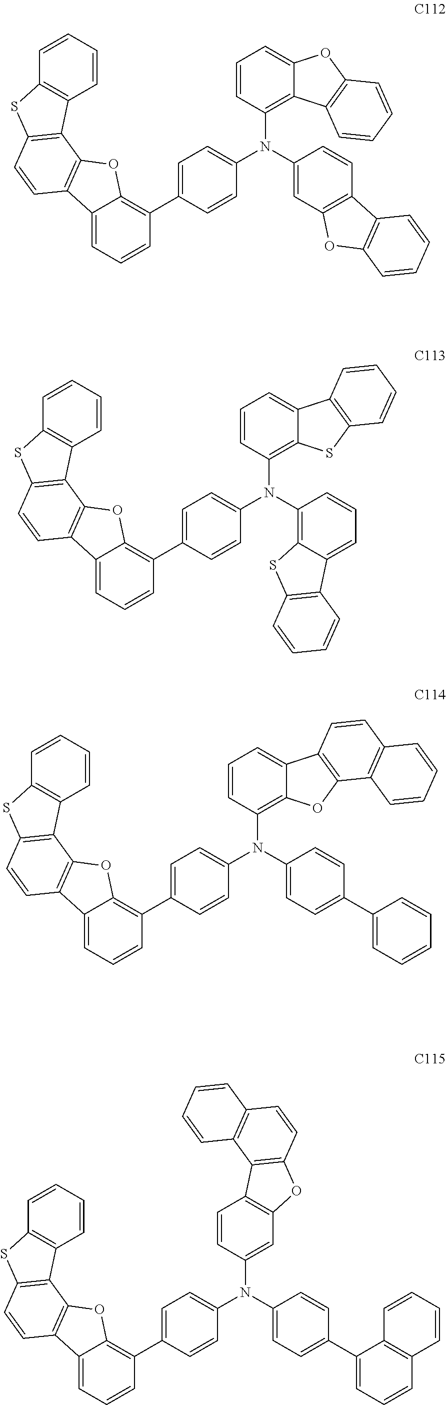 [SMILES: c1ccc2c(c1)oc1cc(N(c3ccc(-c4cccc5c4oc4c5ccc5sc6ccccc6c54)cc3)c3cccc4oc5ccccc5c34)ccc12 ; c1ccc2c(c1)sc1c(N(c3ccc(-c4cccc5c4oc4c5ccc5sc6ccccc6c54)cc3)c3cccc4c3sc3ccccc34)cccc12 ; c1ccc(-c2ccc(N(c3ccc(-c4cccc5c4oc4c5ccc5sc6ccccc6c54)cc3)c3cccc4c3oc3c5ccccc5ccc43)cc2)cc1 ; c1ccc2c(-c3ccc(N(c4ccc(-c5cccc6c5oc5c6ccc6sc7ccccc7c65)cc4)c4ccc5c(c4)oc4ccc6ccccc6c45)cc3)cccc2c1]

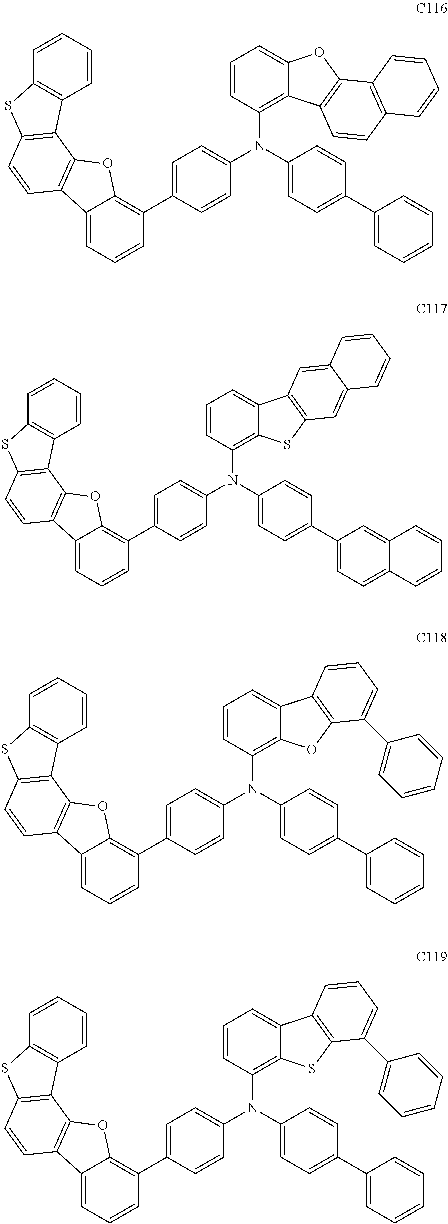 [SMILES: c1ccc(-c2ccc(N(c3ccc(-c4cccc5c4oc4c5ccc5sc6ccccc6c54)cc3)c3cccc4oc5c6ccccc6ccc5c34)cc2)cc1 ; c1ccc2cc(-c3ccc(N(c4ccc(-c5cccc6c5oc5c6ccc6sc7ccccc7c65)cc4)c4cccc5c4sc4cc6ccccc6cc45)cc3)ccc2c1 ; c1ccc(-c2ccc(N(c3ccc(-c4cccc5c4oc4c5ccc5sc6ccccc6c54)cc3)c3cccc4c3oc3c(-c5ccccc5)cccc34)cc2)cc1 ; c1ccc(-c2ccc(N(c3ccc(-c4cccc5c4oc4c5ccc5sc6ccccc6c54)cc3)c3cccc4c3sc3c(-c5ccccc5)cccc34)cc2)cc1]

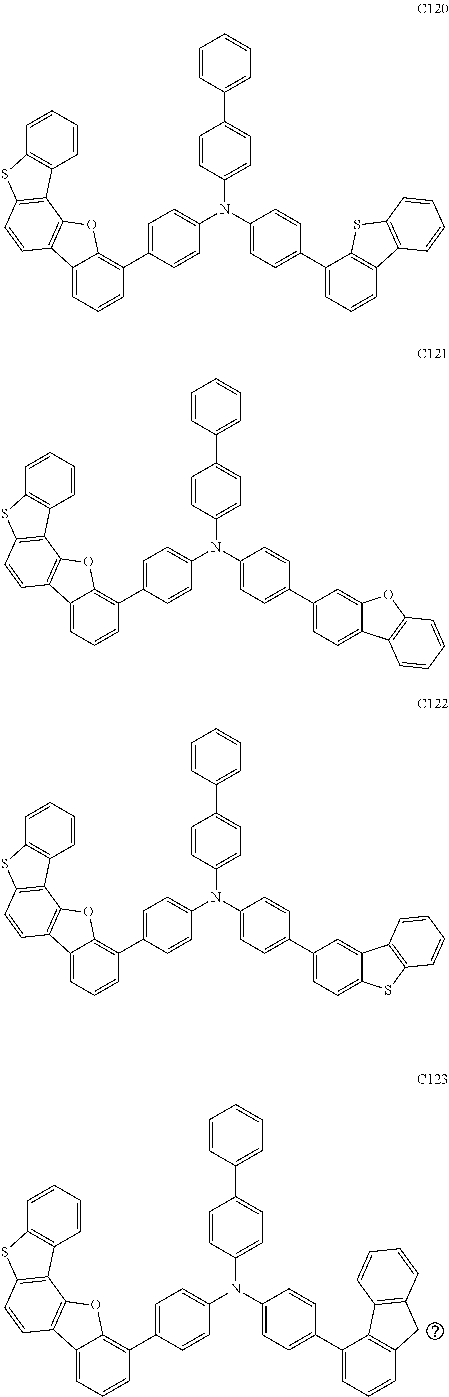 [SMILES: c1ccc(-c2ccc(N(c3ccc(-c4cccc5c4oc4c5ccc5sc6ccccc6c54)cc3)c3ccc(-c4cccc5c4sc4ccccc45)cc3)cc2)cc1 ; c1ccc(-c2ccc(N(c3ccc(-c4ccc5c(c4)oc4ccccc45)cc3)c3ccc(-c4cccc5c4oc4c5ccc5sc6ccccc6c54)cc3)cc2)cc1 ; c1ccc(-c2ccc(N(c3ccc(-c4ccc5sc6ccccc6c5c4)cc3)c3ccc(-c4cccc5c4oc4c5ccc5sc6ccccc6c54)cc3)cc2)cc1 ; c1ccc(-c2ccc(N(c3ccc(-c4cccc5c4-c4ccccc4C5)cc3)c3ccc(-c4cccc5c4oc4c5ccc5sc6ccccc6c54)cc3)cc2)cc1]

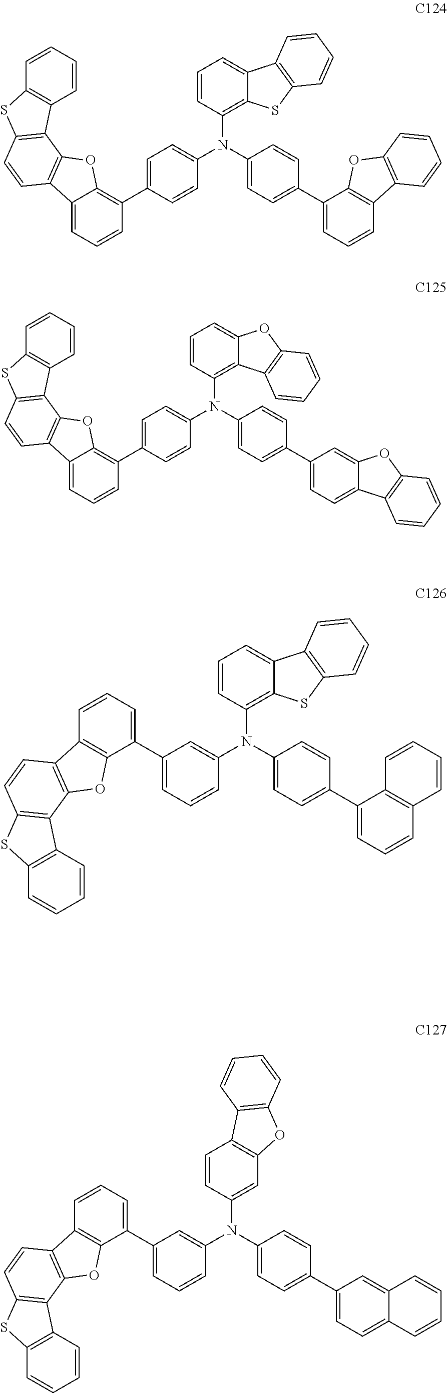 [SMILES: c1ccc2c(c1)oc1c(-c3ccc(N(c4ccc(-c5cccc6c5oc5c6ccc6sc7ccccc7c65)cc4)c4cccc5c4sc4ccccc45)cc3)cccc12 ; c1ccc2c(c1)oc1cc(-c3ccc(N(c4ccc(-c5cccc6c5oc5c6ccc6sc7ccccc7c65)cc4)c4cccc5oc6ccccc6c45)cc3)ccc12 ; c1cc(-c2cccc3c2oc2c3ccc3sc4ccccc4c32)cc(N(c2ccc(-c3cccc4ccccc34)cc2)c2cccc3c2sc2ccccc23)c1 ; c1cc(-c2cccc3c2oc2c3ccc3sc4ccccc4c32)cc(N(c2ccc(-c3ccc4ccccc4c3)cc2)c2ccc3c(c2)oc2ccccc23)c1]

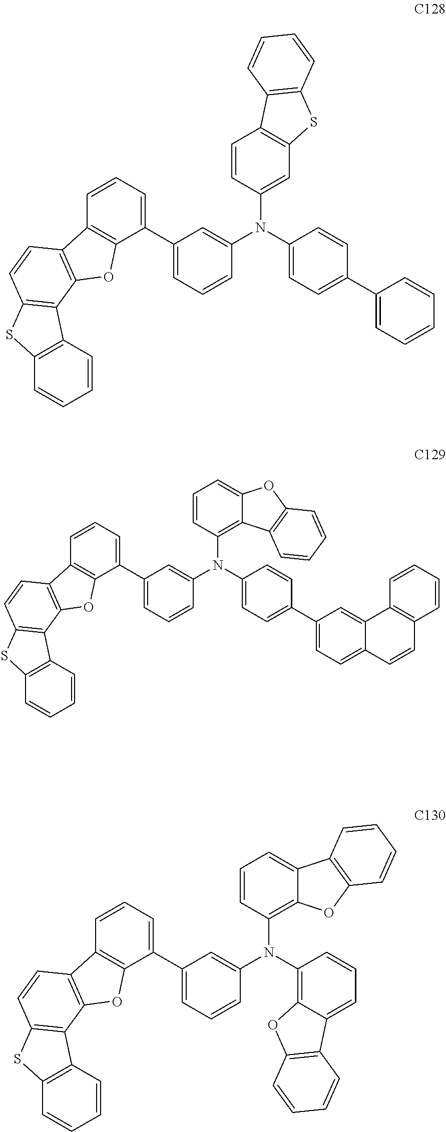 [SMILES: c1ccc(-c2ccc(N(c3cccc(-c4cccc5c4oc4c5ccc5sc6ccccc6c54)c3)c3ccc4c(c3)sc3ccccc34)cc2)cc1 ; c1cc(-c2cccc3c2oc2c3ccc3sc4ccccc4c32)cc(N(c2ccc(-c3ccc4ccc5ccccc5c4c3)cc2)c2cccc3oc4ccccc4c23)c1 ; c1cc(-c2cccc3c2oc2c3ccc3sc4ccccc4c32)cc(N(c2cccc3c2oc2ccccc23)c2cccc3c2oc2ccccc23)c1]

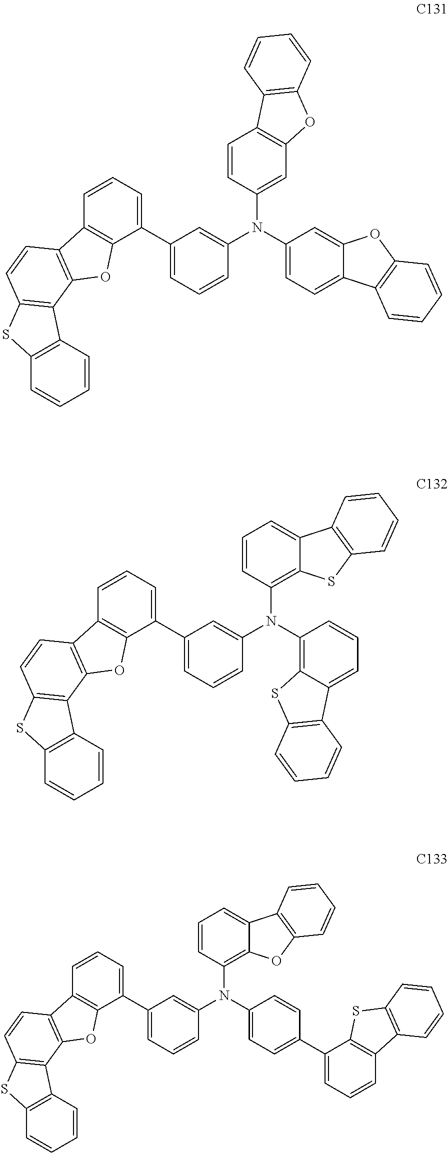 [SMILES: c1cc(-c2cccc3c2oc2c3ccc3sc4ccccc4c32)cc(N(c2ccc3c(c2)oc2ccccc23)c2ccc3c(c2)oc2ccccc23)c1 ; c1cc(-c2cccc3c2oc2c3ccc3sc4ccccc4c32)cc(N(c2cccc3c2sc2ccccc23)c2cccc3c2sc2ccccc23)c1 ; c1cc(-c2cccc3c2oc2c3ccc3sc4ccccc4c32)cc(N(c2ccc(-c3cccc4c3sc3ccccc34)cc2)c2cccc3c2oc2ccccc23)c1]

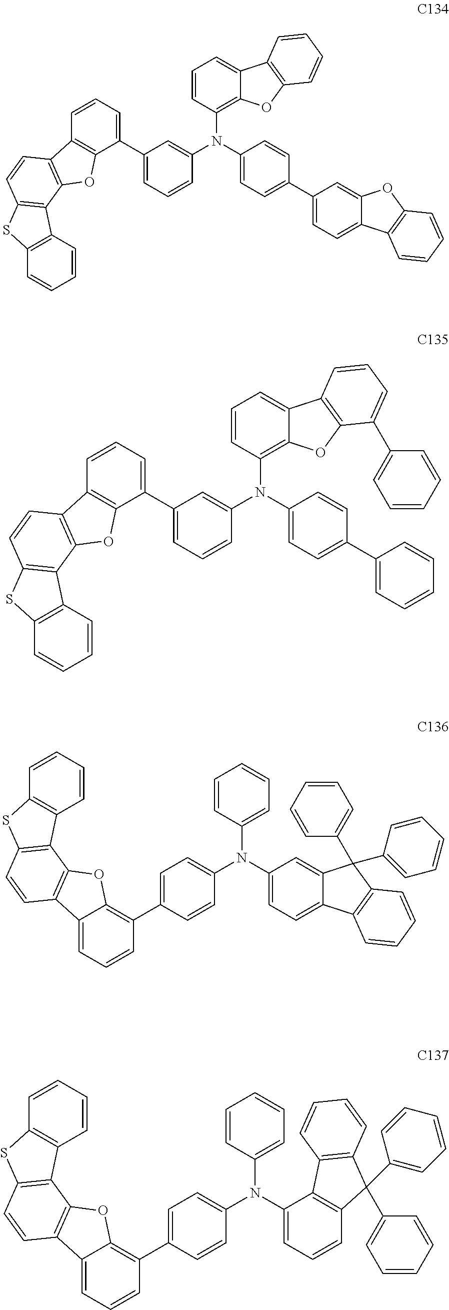 [SMILES: c1cc(-c2cccc3c2oc2c3ccc3sc4ccccc4c32)cc(N(c2ccc(-c3ccc4c(c3)oc3ccccc34)cc2)c2cccc3c2oc2ccccc23)c1 ; c1ccc(-c2ccc(N(c3cccc(-c4cccc5c4oc4c5ccc5sc6ccccc6c54)c3)c3cccc4c3oc3c(-c5ccccc5)cccc34)cc2)cc1 ; c1ccc(N(c2ccc(-c3cccc4c3oc3c4ccc4sc5ccccc5c43)cc2)c2ccc3c(c2)C(c2ccccc2)(c2ccccc2)c2ccccc2-3)cc1 ; c1ccc(N(c2ccc(-c3cccc4c3oc3c4ccc4sc5ccccc5c43)cc2)c2cccc3c2-c2ccccc2C3(c2ccccc2)c2ccccc2)cc1]

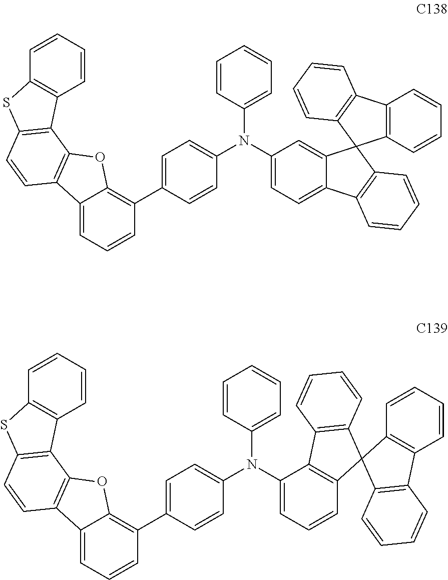 [SMILES: c1ccc(N(c2ccc(-c3cccc4c3oc3c4ccc4sc5ccccc5c43)cc2)c2ccc3c(c2)C2(c4ccccc4-c4ccccc42)c2ccccc2-3)cc1 ; c1ccc(N(c2ccc(-c3cccc4c3oc3c4ccc4sc5ccccc5c43)cc2)c2cccc3c2-c2ccccc2C32c3ccccc3-c3ccccc32)cc1]

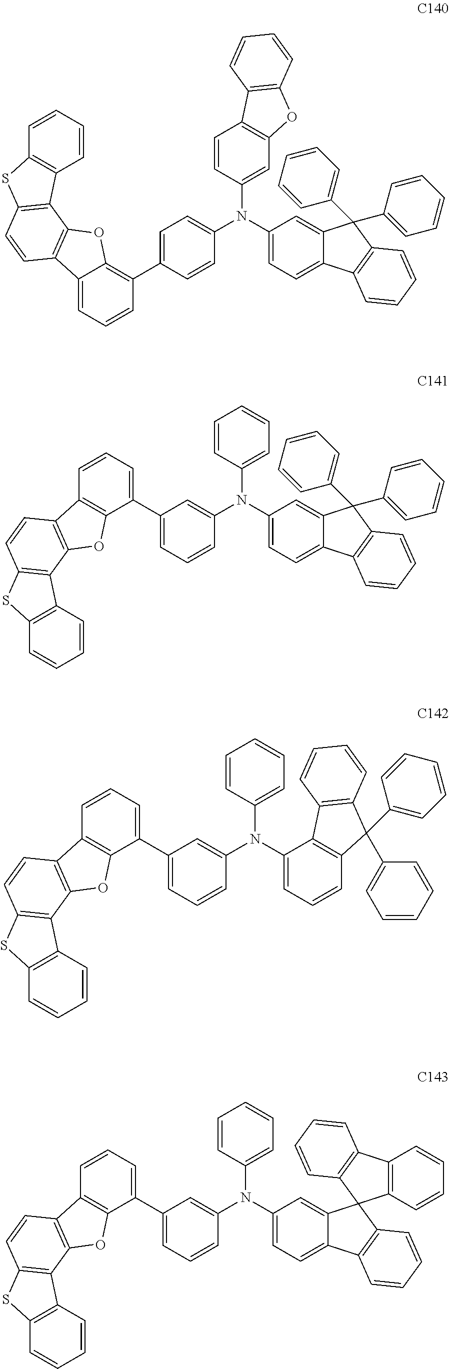 [SMILES: c1ccc(C2(c3ccccc3)c3ccccc3-c3ccc(N(c4ccc(-c5cccc6c5oc5c6ccc6sc7ccccc7c65)cc4)c4ccc5c(c4)oc4ccccc45)cc32)cc1 ; c1ccc(N(c2cccc(-c3cccc4c3oc3c4ccc4sc5ccccc5c43)c2)c2ccc3c(c2)C(c2ccccc2)(c2ccccc2)c2ccccc2-3)cc1 ; c1ccc(N(c2cccc(-c3cccc4c3oc3c4ccc4sc5ccccc5c43)c2)c2cccc3c2-c2ccccc2C3(c2ccccc2)c2ccccc2)cc1 ; c1ccc(N(c2cccc(-c3cccc4c3oc3c4ccc4sc5ccccc5c43)c2)c2ccc3c(c2)C2(c4ccccc4-c4ccccc42)c2ccccc2-3)cc1]

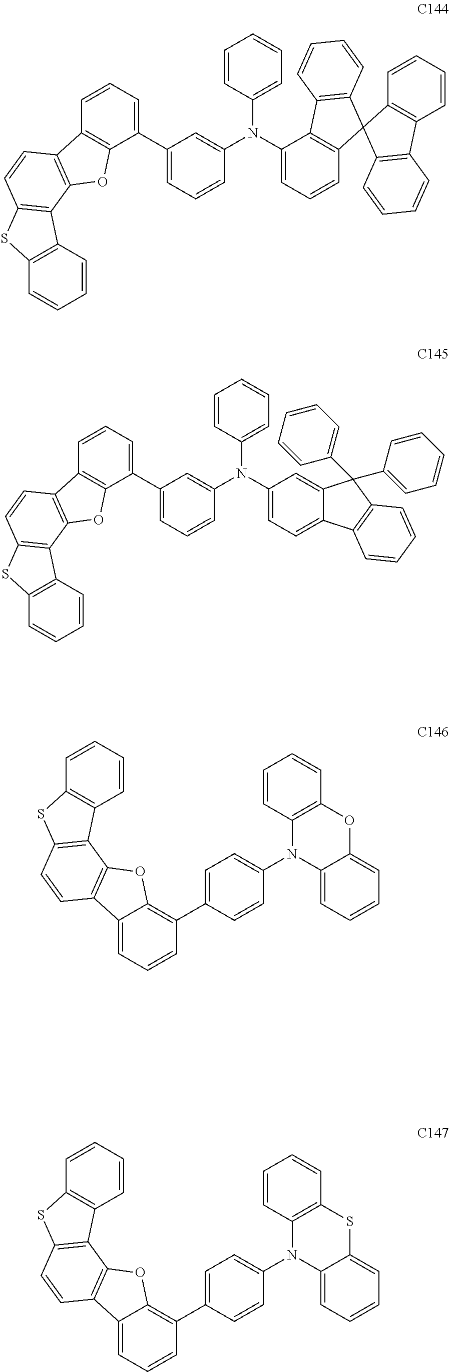 [SMILES: c1ccc(N(c2cccc(-c3cccc4c3oc3c4ccc4sc5ccccc5c43)c2)c2cccc3c2-c2ccccc2C32c3ccccc3-c3ccccc32)cc1 ; c1ccc(N(c2cccc(-c3cccc4c3oc3c4ccc4sc5ccccc5c43)c2)c2ccc3c(c2)C(c2ccccc2)(c2ccccc2)c2ccccc2-3)cc1 ; c1ccc2c(c1)Oc1ccccc1N2c1ccc(-c2cccc3c2oc2c3ccc3sc4ccccc4c32)cc1 ; c1ccc2c(c1)Sc1ccccc1N2c1ccc(-c2cccc3c2oc2c3ccc3sc4ccccc4c32)cc1]

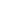

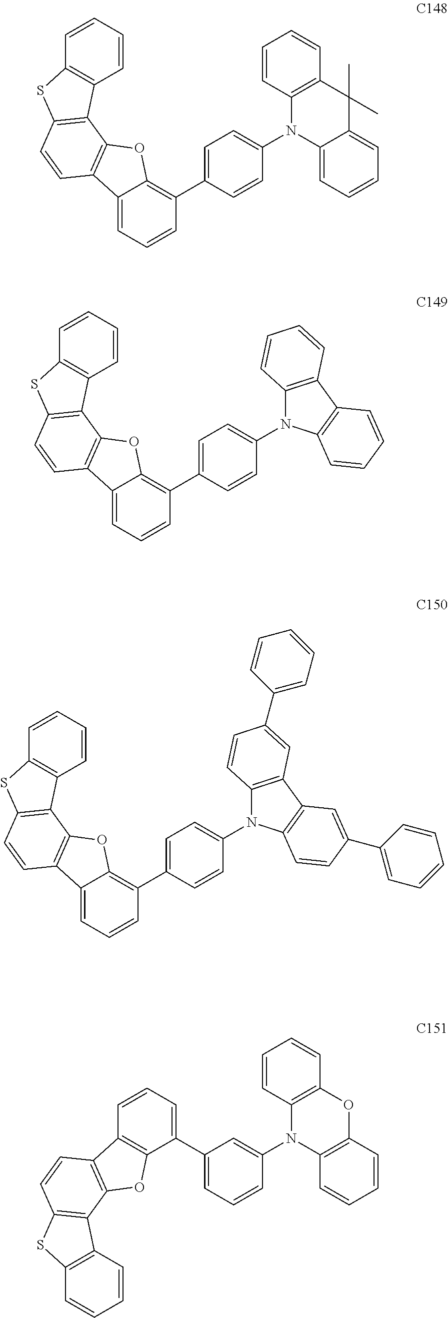 [SMILES: CC1(C)c2ccccc2N(c2ccc(-c3cccc4c3oc3c4ccc4sc5ccccc5c43)cc2)c2ccccc21 ; c1ccc2c(c1)sc1ccc3c4cccc(-c5ccc(-n6c7ccccc7c7ccccc76)cc5)c4oc3c12 ; c1ccc(-c2ccc3c(c2)c2cc(-c4ccccc4)ccc2n3-c2ccc(-c3cccc4c3oc3c4ccc4sc5ccccc5c43)cc2)cc1 ; c1cc(-c2cccc3c2oc2c3ccc3sc4ccccc4c32)cc(N2c3ccccc3Oc3ccccc32)c1]

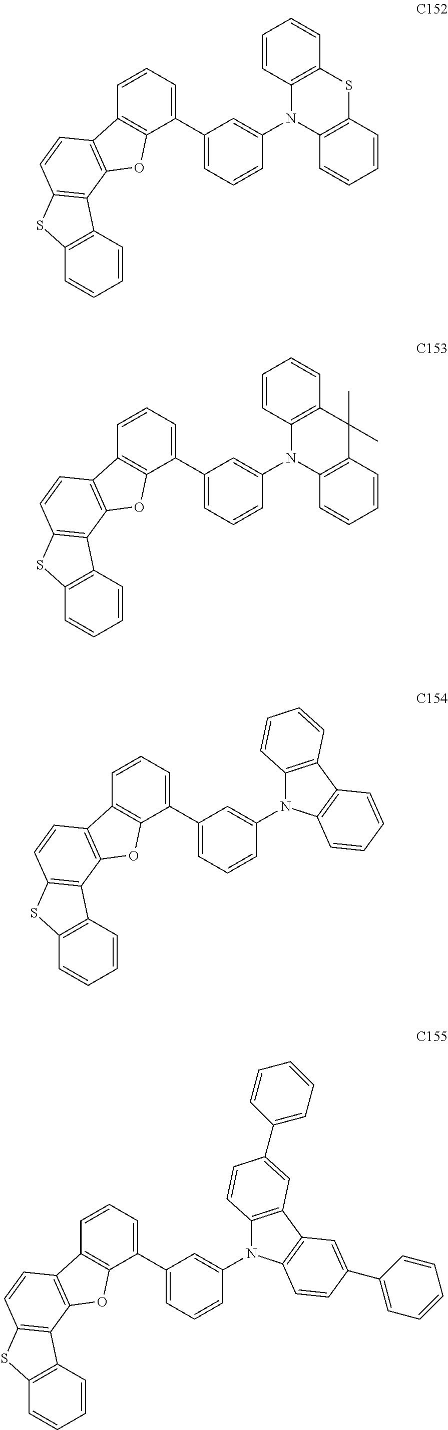 [SMILES: c1cc(-c2cccc3c2oc2c3ccc3sc4ccccc4c32)cc(N2c3ccccc3Sc3ccccc32)c1 ; CC1(C)c2ccccc2N(c2cccc(-c3cccc4c3oc3c4ccc4sc5ccccc5c43)c2)c2ccccc21 ; c1cc(-c2cccc3c2oc2c3ccc3sc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3oc3c4ccc4sc5ccccc5c43)c2)cc1]

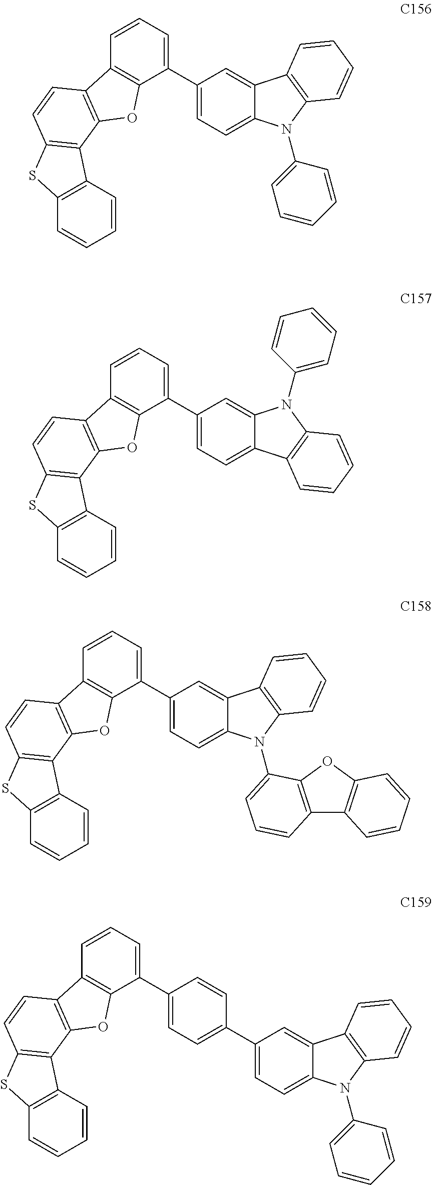 [SMILES: c1ccc(-n2c3ccccc3c3cc(-c4cccc5c4oc4c5ccc5sc6ccccc6c54)ccc32)cc1 ; c1ccc(-n2c3ccccc3c3ccc(-c4cccc5c4oc4c5ccc5sc6ccccc6c54)cc32)cc1 ; c1ccc2c(c1)oc1c(-n3c4ccccc4c4cc(-c5cccc6c5oc5c6ccc6sc7ccccc7c65)ccc43)cccc12 ; c1ccc(-n2c3ccccc3c3cc(-c4ccc(-c5cccc6c5oc5c6ccc6sc7ccccc7c65)cc4)ccc32)cc1]

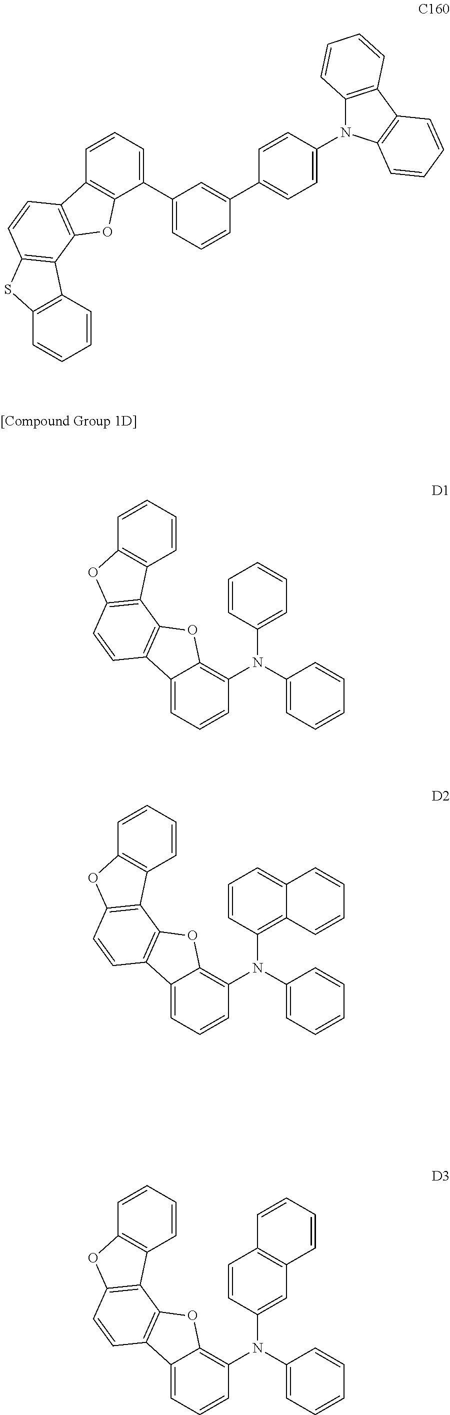 [SMILES: c1cc(-c2ccc(-n3c4ccccc4c4ccccc43)cc2)cc(-c2cccc3c2oc2c3ccc3sc4ccccc4c32)c1 ; c1ccc(N(c2ccccc2)c2cccc3c2oc2c3ccc3oc4ccccc4c32)cc1 ; c1ccc(N(c2cccc3ccccc23)c2cccc3c2oc2c3ccc3oc4ccccc4c32)cc1 ; c1ccc(N(c2ccc3ccccc3c2)c2cccc3c2oc2c3ccc3oc4ccccc4c32)cc1]

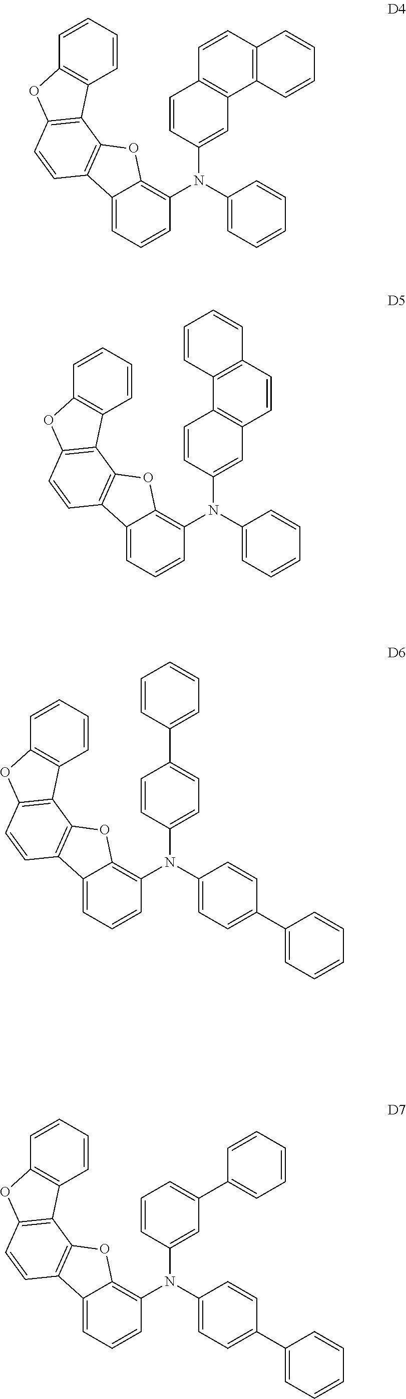 [SMILES: c1ccc(N(c2ccc3ccc4ccccc4c3c2)c2cccc3c2oc2c3ccc3oc4ccccc4c32)cc1 ; c1ccc(N(c2ccc3c(ccc4ccccc43)c2)c2cccc3c2oc2c3ccc3oc4ccccc4c32)cc1 ; c1ccc(-c2ccc(N(c3ccc(-c4ccccc4)cc3)c3cccc4c3oc3c4ccc4oc5ccccc5c43)cc2)cc1 ; c1ccc(-c2ccc(N(c3cccc(-c4ccccc4)c3)c3cccc4c3oc3c4ccc4oc5ccccc5c43)cc2)cc1]

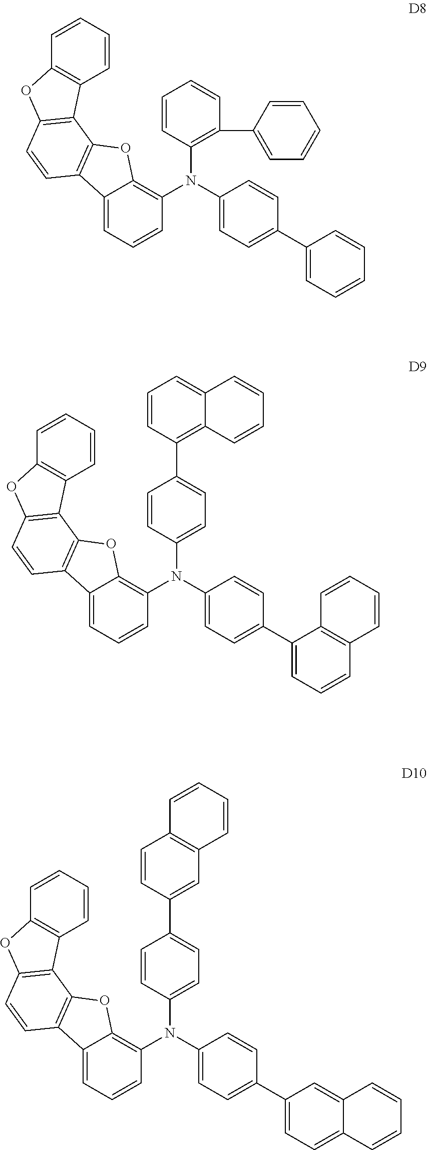 [SMILES: c1ccc(-c2ccc(N(c3ccccc3-c3ccccc3)c3cccc4c3oc3c4ccc4oc5ccccc5c43)cc2)cc1 ; c1ccc2c(-c3ccc(N(c4ccc(-c5cccc6ccccc56)cc4)c4cccc5c4oc4c5ccc5oc6ccccc6c54)cc3)cccc2c1 ; c1ccc2cc(-c3ccc(N(c4ccc(-c5ccc6ccccc6c5)cc4)c4cccc5c4oc4c5ccc5oc6ccccc6c54)cc3)ccc2c1]

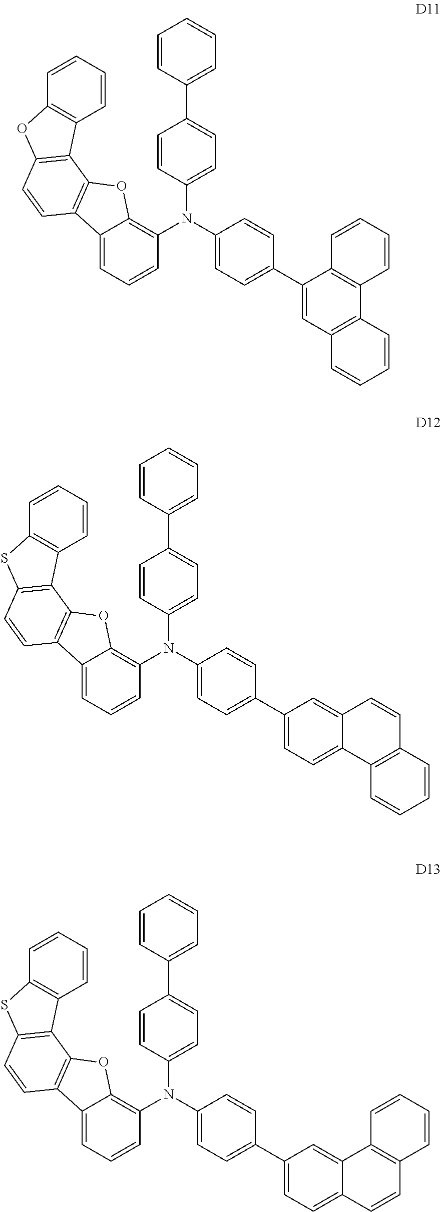 [SMILES: c1ccc(-c2ccc(N(c3ccc(-c4cc5ccccc5c5ccccc45)cc3)c3cccc4c3oc3c4ccc4oc5ccccc5c43)cc2)cc1 ; c1ccc(-c2ccc(N(c3ccc(-c4ccc5c(ccc6ccccc65)c4)cc3)c3cccc4c3oc3c4ccc4sc5ccccc5c43)cc2)cc1 ; c1ccc(-c2ccc(N(c3ccc(-c4ccc5ccc6ccccc6c5c4)cc3)c3cccc4c3oc3c4ccc4sc5ccccc5c43)cc2)cc1]

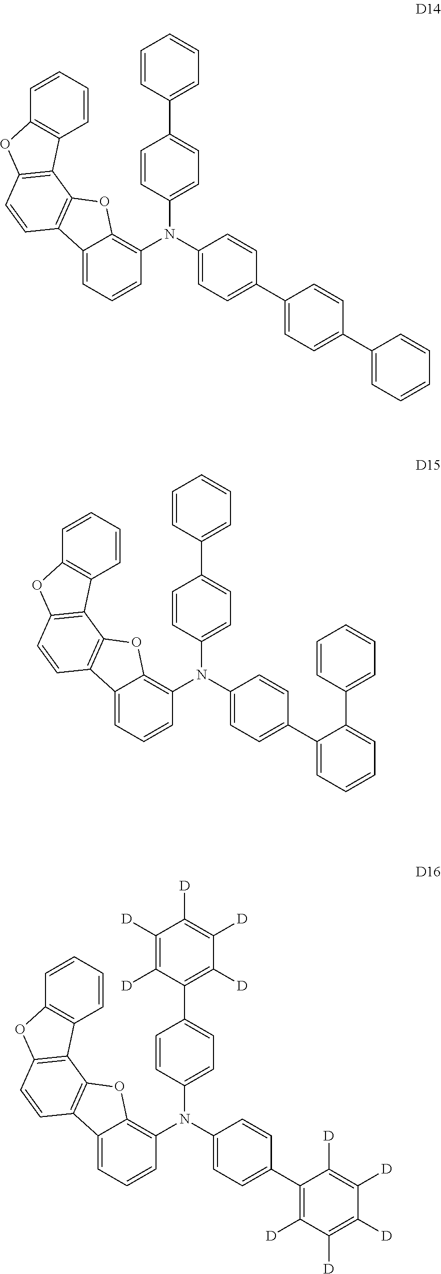 [SMILES: c1ccc(-c2ccc(-c3ccc(N(c4ccc(-c5ccccc5)cc4)c4cccc5c4oc4c5ccc5oc6ccccc6c54)cc3)cc2)cc1 ; c1ccc(-c2ccc(N(c3ccc(-c4ccccc4-c4ccccc4)cc3)c3cccc4c3oc3c4ccc4oc5ccccc5c43)cc2)cc1 ; [2H]c1c([2H])c([2H])c(-c2ccc(N(c3ccc(-c4c([2H])c([2H])c([2H])c([2H])c4[2H])cc3)c3cccc4c3oc3c4ccc4oc5ccccc5c43)cc2)c([2H])c1[2H]]

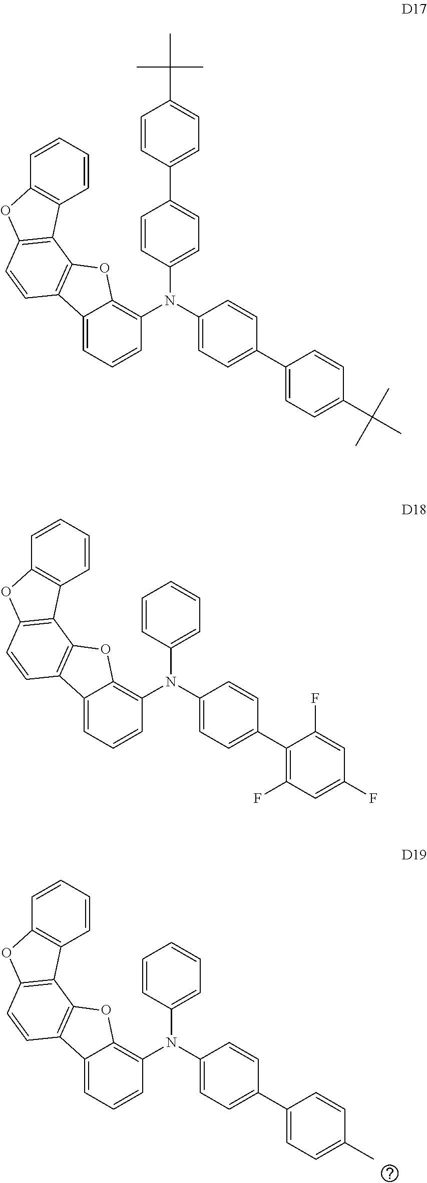 [SMILES: CC(C)(C)c1ccc(-c2ccc(N(c3ccc(-c4ccc(C(C)(C)C)cc4)cc3)c3cccc4c3oc3c4ccc4oc5ccccc5c43)cc2)cc1 ; Fc1cc(F)c(-c2ccc(N(c3ccccc3)c3cccc4c3oc3c4ccc4oc5ccccc5c43)cc2)c(F)c1 ; Cc1ccc(-c2ccc(N(c3ccccc3)c3cccc4c3oc3c4ccc4oc5ccccc5c43)cc2)cc1]

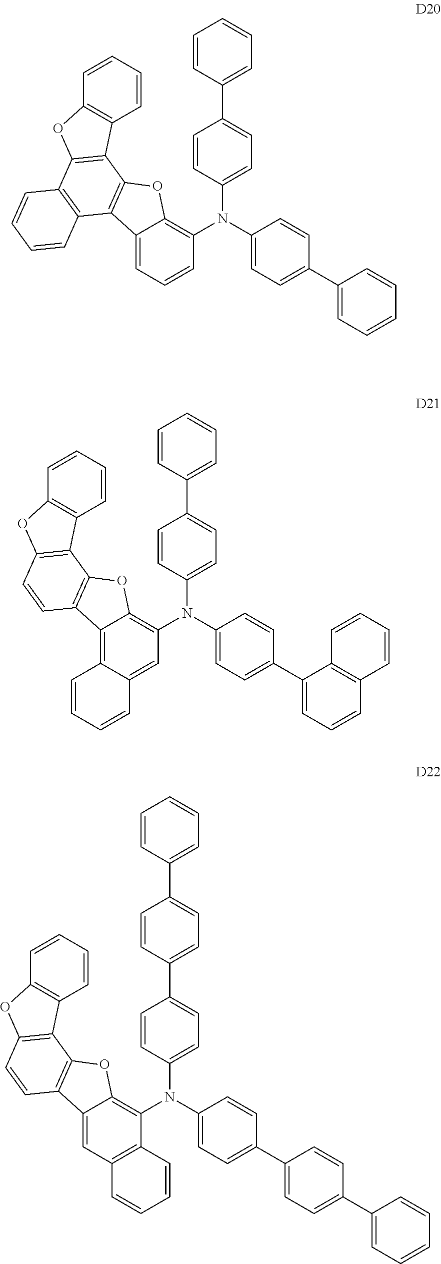 [SMILES: c1ccc(-c2ccc(N(c3ccc(-c4ccccc4)cc3)c3cccc4c3oc3c4c4ccccc4c4oc5ccccc5c43)cc2)cc1 ; c1ccc(-c2ccc(N(c3ccc(-c4cccc5ccccc45)cc3)c3cc4ccccc4c4c3oc3c4ccc4oc5ccccc5c43)cc2)cc1 ; c1ccc(-c2ccc(-c3ccc(N(c4ccc(-c5ccc(-c6ccccc6)cc5)cc4)c4c5ccccc5cc5c4oc4c5ccc5oc6ccccc6c54)cc3)cc2)cc1]

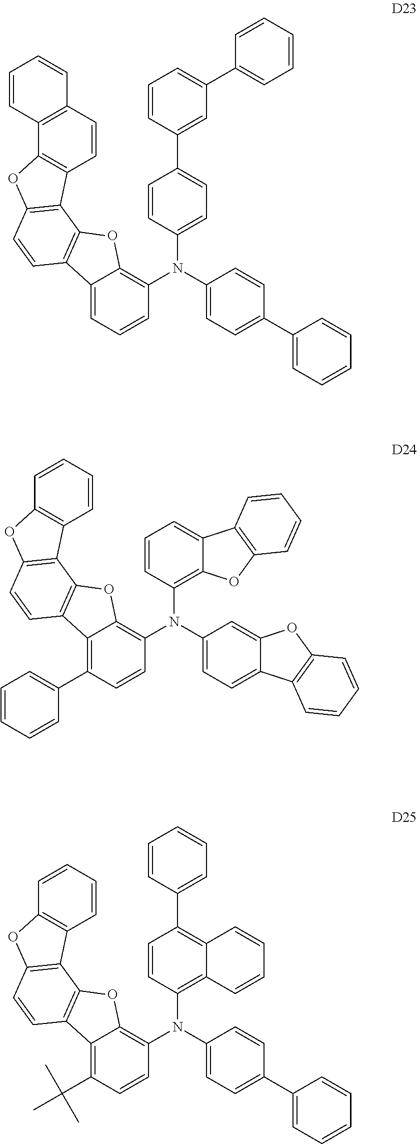 [SMILES: c1ccc(-c2ccc(N(c3ccc(-c4cccc(-c5ccccc5)c4)cc3)c3cccc4c3oc3c4ccc4oc5c6ccccc6ccc5c43)cc2)cc1 ; c1ccc(-c2ccc(N(c3ccc4c(c3)oc3ccccc34)c3cccc4c3oc3ccccc34)c3oc4c(ccc5oc6ccccc6c54)c23)cc1 ; CC(C)(C)c1ccc(N(c2ccc(-c3ccccc3)cc2)c2ccc(-c3ccccc3)c3ccccc23)c2oc3c(ccc4oc5ccccc5c43)c12]

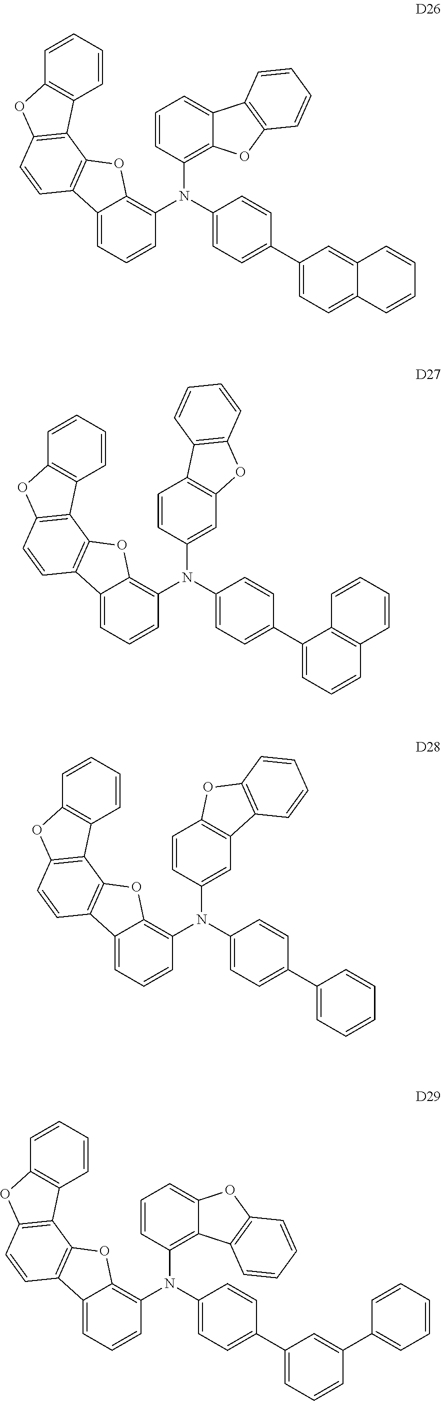 [SMILES: c1ccc2cc(-c3ccc(N(c4cccc5c4oc4ccccc45)c4cccc5c4oc4c5ccc5oc6ccccc6c54)cc3)ccc2c1 ; c1ccc2c(-c3ccc(N(c4ccc5c(c4)oc4ccccc45)c4cccc5c4oc4c5ccc5oc6ccccc6c54)cc3)cccc2c1 ; c1ccc(-c2ccc(N(c3ccc4oc5ccccc5c4c3)c3cccc4c3oc3c4ccc4oc5ccccc5c43)cc2)cc1 ; c1ccc(-c2cccc(-c3ccc(N(c4cccc5c4oc4c5ccc5oc6ccccc6c54)c4cccc5oc6ccccc6c45)cc3)c2)cc1]

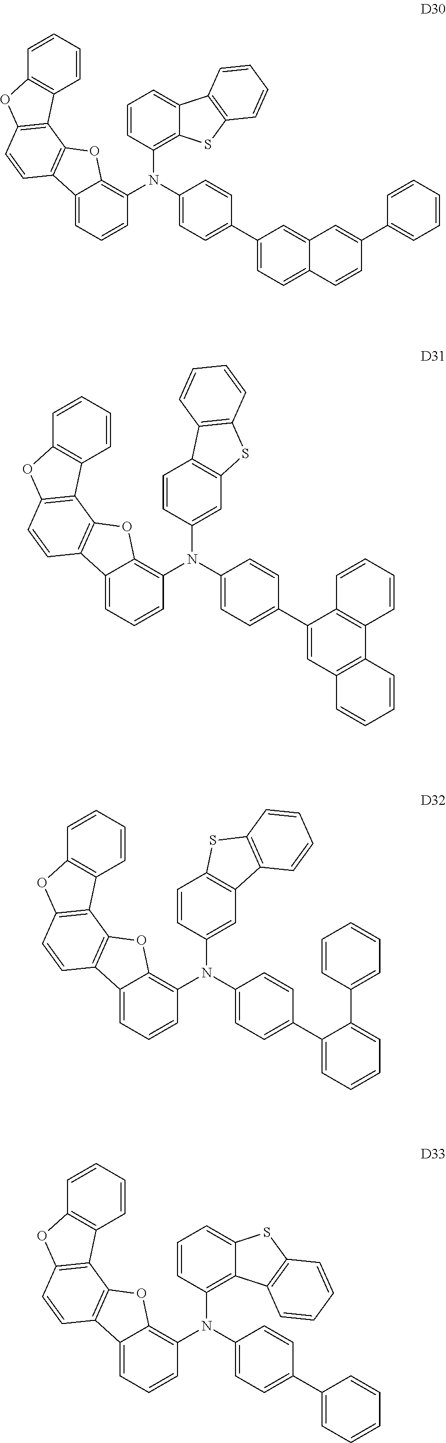 [SMILES: c1ccc(-c2ccc3ccc(-c4ccc(N(c5cccc6c5oc5c6ccc6oc7ccccc7c65)c5cccc6c5sc5ccccc56)cc4)cc3c2)cc1 ; c1ccc2c(c1)cc(-c1ccc(N(c3ccc4c(c3)sc3ccccc34)c3cccc4c3oc3c4ccc4oc5ccccc5c43)cc1)c1ccccc12 ; c1ccc(-c2ccccc2-c2ccc(N(c3ccc4sc5ccccc5c4c3)c3cccc4c3oc3c4ccc4oc5ccccc5c43)cc2)cc1 ; c1ccc(-c2ccc(N(c3cccc4c3oc3c4ccc4oc5ccccc5c43)c3cccc4sc5ccccc5c34)cc2)cc1]

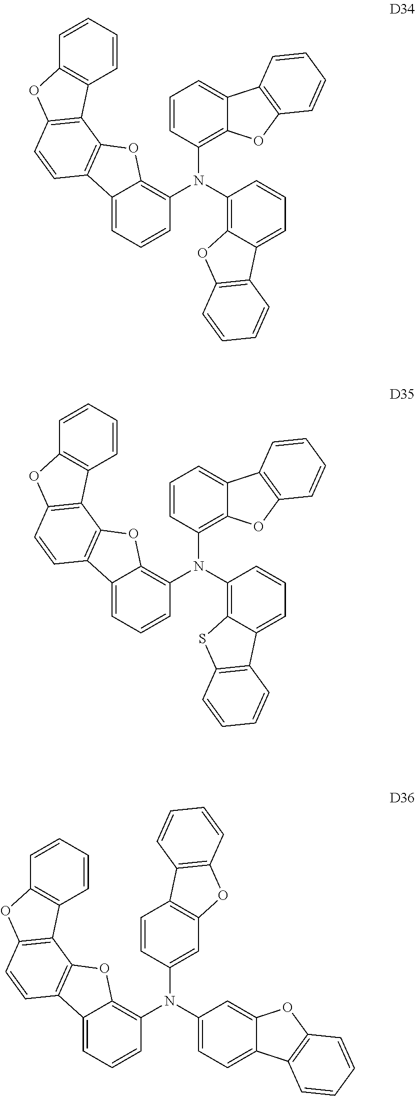 [SMILES: c1ccc2c(c1)oc1c(N(c3cccc4c3oc3ccccc34)c3cccc4c3oc3c4ccc4oc5ccccc5c43)cccc12 ; c1ccc2c(c1)oc1c(N(c3cccc4c3oc3c4ccc4oc5ccccc5c43)c3cccc4c3sc3ccccc34)cccc12 ; c1ccc2c(c1)oc1cc(N(c3ccc4c(c3)oc3ccccc34)c3cccc4c3oc3c4ccc4oc5ccccc5c43)ccc12]

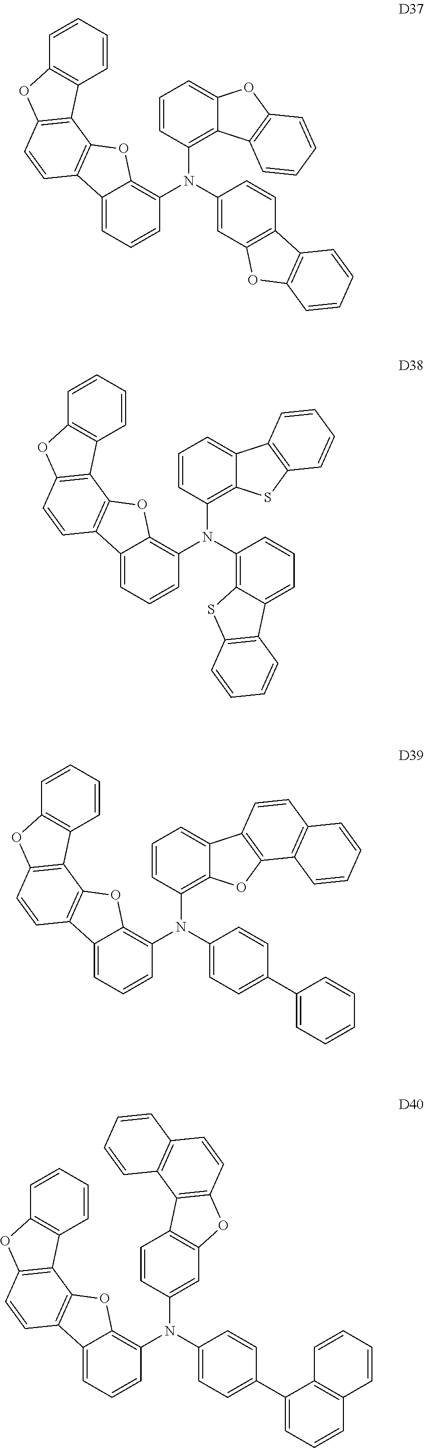 [SMILES: c1ccc2c(c1)oc1cc(N(c3cccc4c3oc3c4ccc4oc5ccccc5c43)c3cccc4oc5ccccc5c34)ccc12 ; c1ccc2c(c1)oc1ccc3c4cccc(N(c5cccc6c5sc5ccccc56)c5cccc6c5sc5ccccc56)c4oc3c12 ; c1ccc(-c2ccc(N(c3cccc4c3oc3c5ccccc5ccc43)c3cccc4c3oc3c4ccc4oc5ccccc5c43)cc2)cc1 ; c1ccc2c(-c3ccc(N(c4ccc5c(c4)oc4ccc6ccccc6c45)c4cccc5c4oc4c5ccc5oc6ccccc6c54)cc3)cccc2c1]

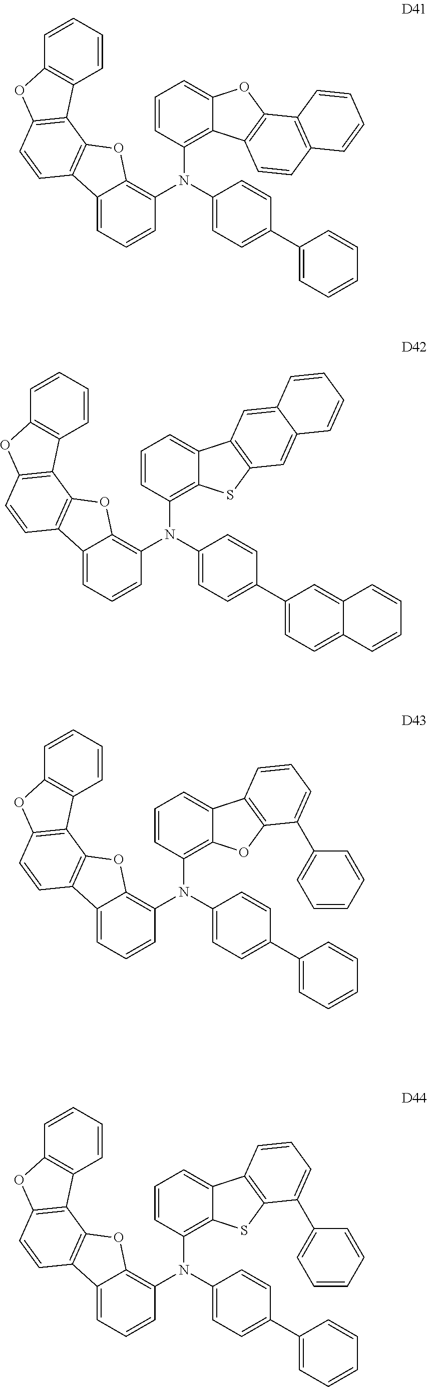 [SMILES: c1ccc(-c2ccc(N(c3cccc4c3oc3c4ccc4oc5ccccc5c43)c3cccc4oc5c6ccccc6ccc5c34)cc2)cc1 ; c1ccc2cc(-c3ccc(N(c4cccc5c4oc4c5ccc5oc6ccccc6c54)c4cccc5c4sc4cc6ccccc6cc45)cc3)ccc2c1 ; c1ccc(-c2ccc(N(c3cccc4c3oc3c(-c5ccccc5)cccc34)c3cccc4c3oc3c4ccc4oc5ccccc5c43)cc2)cc1 ; c1ccc(-c2ccc(N(c3cccc4c3oc3c4ccc4oc5ccccc5c43)c3cccc4c3sc3c(-c5ccccc5)cccc34)cc2)cc1]

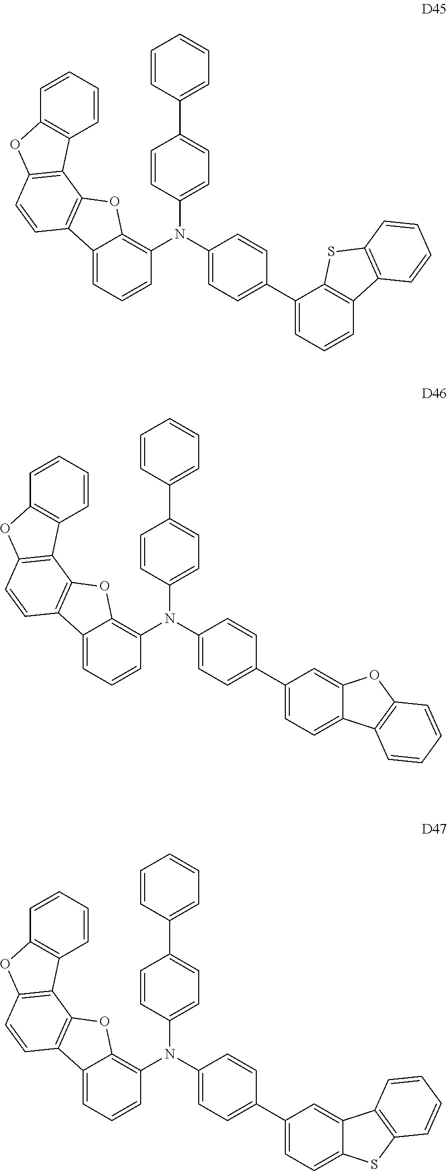 [SMILES: c1ccc(-c2ccc(N(c3ccc(-c4cccc5c4sc4ccccc45)cc3)c3cccc4c3oc3c4ccc4oc5ccccc5c43)cc2)cc1 ; c1ccc(-c2ccc(N(c3ccc(-c4ccc5c(c4)oc4ccccc45)cc3)c3cccc4c3oc3c4ccc4oc5ccccc5c43)cc2)cc1 ; c1ccc(-c2ccc(N(c3ccc(-c4ccc5sc6ccccc6c5c4)cc3)c3cccc4c3oc3c4ccc4oc5ccccc5c43)cc2)cc1]

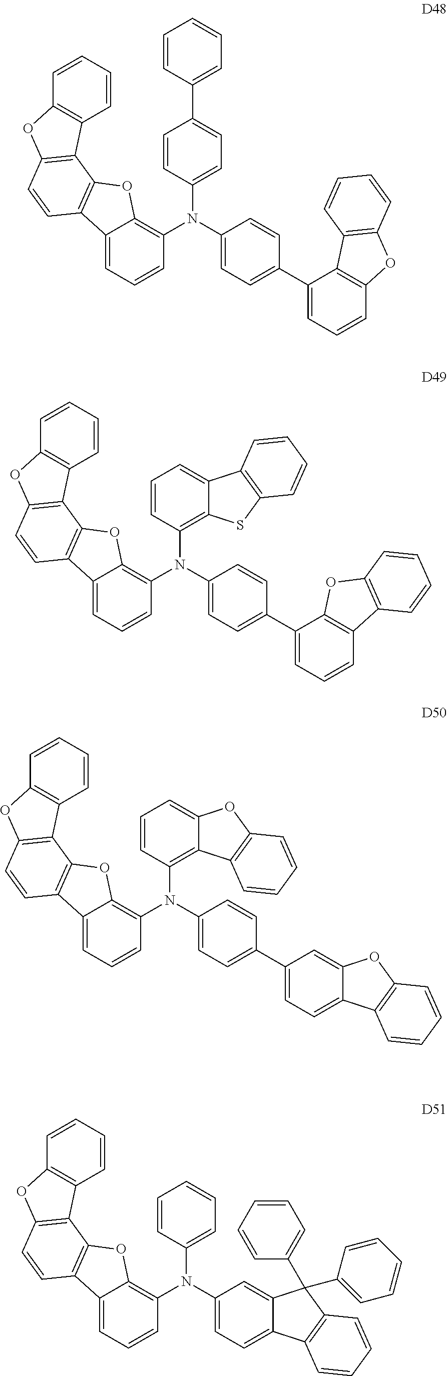 [SMILES: c1ccc(-c2ccc(N(c3ccc(-c4cccc5oc6ccccc6c45)cc3)c3cccc4c3oc3c4ccc4oc5ccccc5c43)cc2)cc1 ; c1ccc2c(c1)oc1c(-c3ccc(N(c4cccc5c4oc4c5ccc5oc6ccccc6c54)c4cccc5c4sc4ccccc45)cc3)cccc12 ; c1ccc2c(c1)oc1cc(-c3ccc(N(c4cccc5c4oc4c5ccc5oc6ccccc6c54)c4cccc5oc6ccccc6c45)cc3)ccc12 ; c1ccc(N(c2ccc3c(c2)C(c2ccccc2)(c2ccccc2)c2ccccc2-3)c2cccc3c2oc2c3ccc3oc4ccccc4c32)cc1]

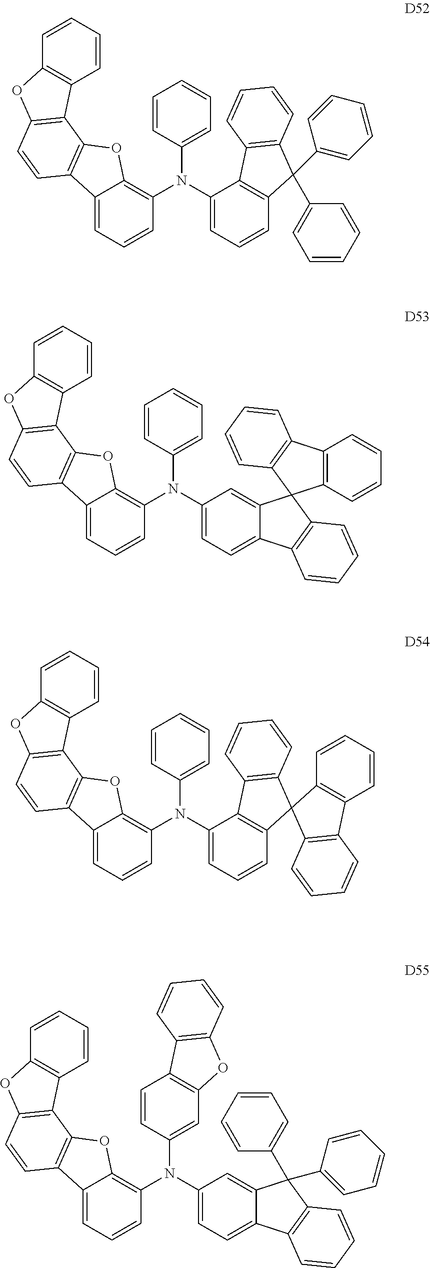 [SMILES: c1ccc(N(c2cccc3c2-c2ccccc2C3(c2ccccc2)c2ccccc2)c2cccc3c2oc2c3ccc3oc4ccccc4c32)cc1 ; c1ccc(N(c2ccc3c(c2)C2(c4ccccc4-c4ccccc42)c2ccccc2-3)c2cccc3c2oc2c3ccc3oc4ccccc4c32)cc1 ; c1ccc(N(c2cccc3c2-c2ccccc2C32c3ccccc3-c3ccccc32)c2cccc3c2oc2c3ccc3oc4ccccc4c32)cc1 ; c1ccc(C2(c3ccccc3)c3ccccc3-c3ccc(N(c4ccc5c(c4)oc4ccccc45)c4cccc5c4oc4c5ccc5oc6ccccc6c54)cc32)cc1]

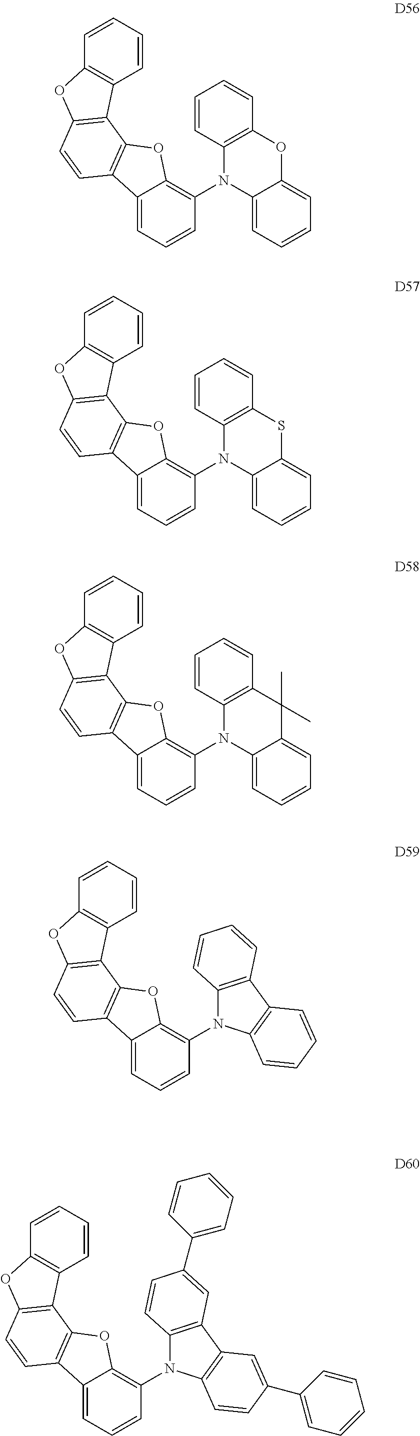 [SMILES: c1ccc2c(c1)Oc1ccccc1N2c1cccc2c1oc1c2ccc2oc3ccccc3c21 ; c1ccc2c(c1)Sc1ccccc1N2c1cccc2c1oc1c2ccc2oc3ccccc3c21 ; CC1(C)c2ccccc2N(c2cccc3c2oc2c3ccc3oc4ccccc4c32)c2ccccc21 ; c1ccc2c(c1)oc1ccc3c4cccc(-n5c6ccccc6c6ccccc65)c4oc3c12 ; c1ccc(-c2ccc3c(c2)c2cc(-c4ccccc4)ccc2n3-c2cccc3c2oc2c3ccc3oc4ccccc4c32)cc1]

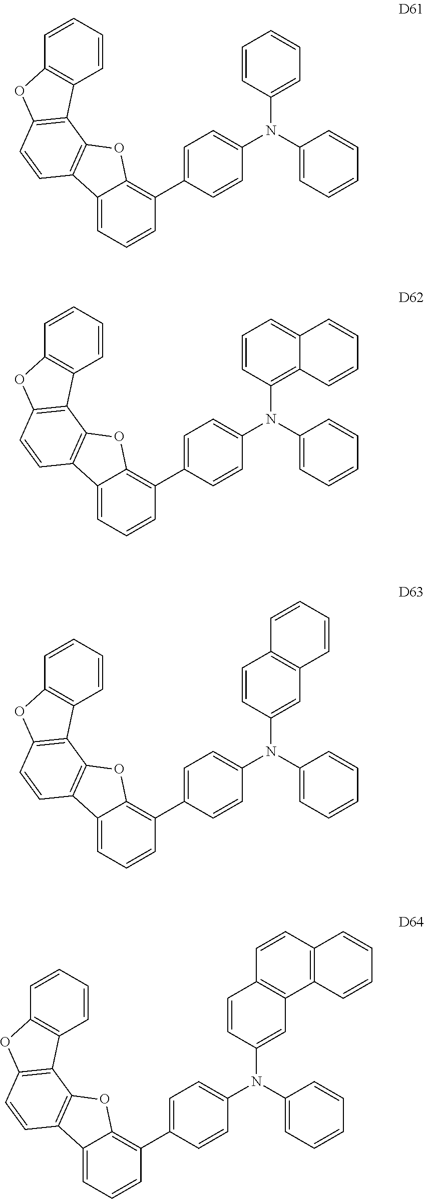 [SMILES: c1ccc(N(c2ccccc2)c2ccc(-c3cccc4c3oc3c4ccc4oc5ccccc5c43)cc2)cc1 ; c1ccc(N(c2ccc(-c3cccc4c3oc3c4ccc4oc5ccccc5c43)cc2)c2cccc3ccccc23)cc1 ; c1ccc(N(c2ccc(-c3cccc4c3oc3c4ccc4oc5ccccc5c43)cc2)c2ccc3ccccc3c2)cc1 ; c1ccc(N(c2ccc(-c3cccc4c3oc3c4ccc4oc5ccccc5c43)cc2)c2ccc3ccc4ccccc4c3c2)cc1]

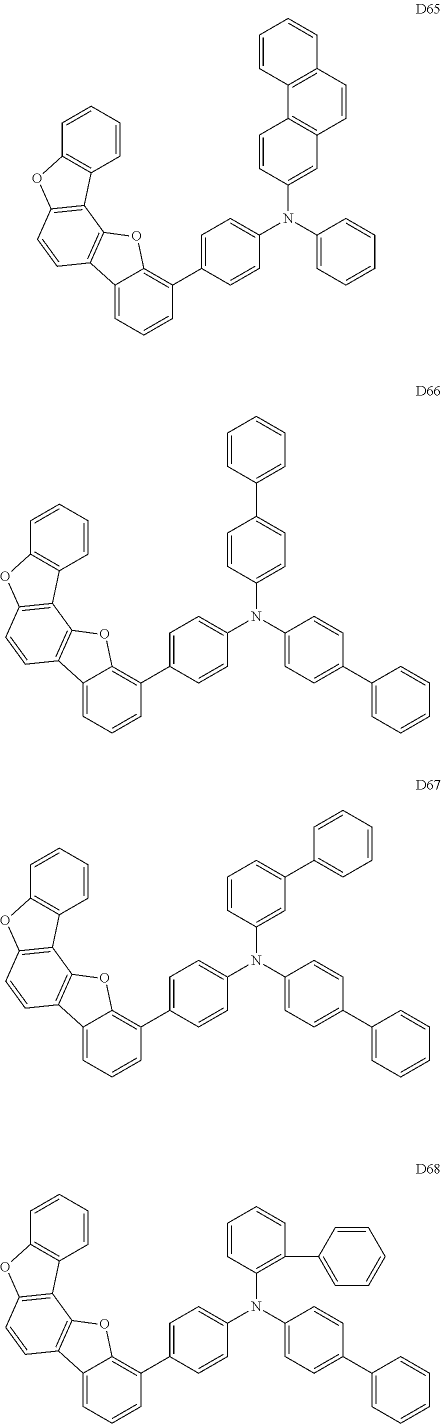 [SMILES: c1ccc(N(c2ccc(-c3cccc4c3oc3c4ccc4oc5ccccc5c43)cc2)c2ccc3c(ccc4ccccc43)c2)cc1 ; c1ccc(-c2ccc(N(c3ccc(-c4ccccc4)cc3)c3ccc(-c4cccc5c4oc4c5ccc5oc6ccccc6c54)cc3)cc2)cc1 ; c1ccc(-c2ccc(N(c3ccc(-c4cccc5c4oc4c5ccc5oc6ccccc6c54)cc3)c3cccc(-c4ccccc4)c3)cc2)cc1 ; c1ccc(-c2ccc(N(c3ccc(-c4cccc5c4oc4c5ccc5oc6ccccc6c54)cc3)c3ccccc3-c3ccccc3)cc2)cc1]

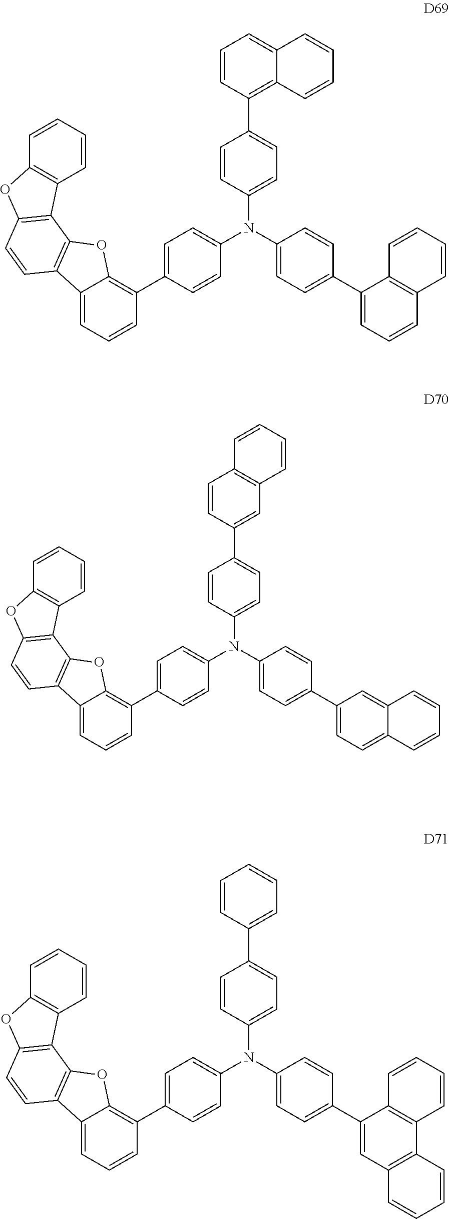 [SMILES: c1ccc2c(-c3ccc(N(c4ccc(-c5cccc6ccccc56)cc4)c4ccc(-c5cccc6c5oc5c6ccc6oc7ccccc7c65)cc4)cc3)cccc2c1 ; c1ccc2cc(-c3ccc(N(c4ccc(-c5ccc6ccccc6c5)cc4)c4ccc(-c5cccc6c5oc5c6ccc6oc7ccccc7c65)cc4)cc3)ccc2c1 ; c1ccc(-c2ccc(N(c3ccc(-c4cc5ccccc5c5ccccc45)cc3)c3ccc(-c4cccc5c4oc4c5ccc5oc6ccccc6c54)cc3)cc2)cc1]

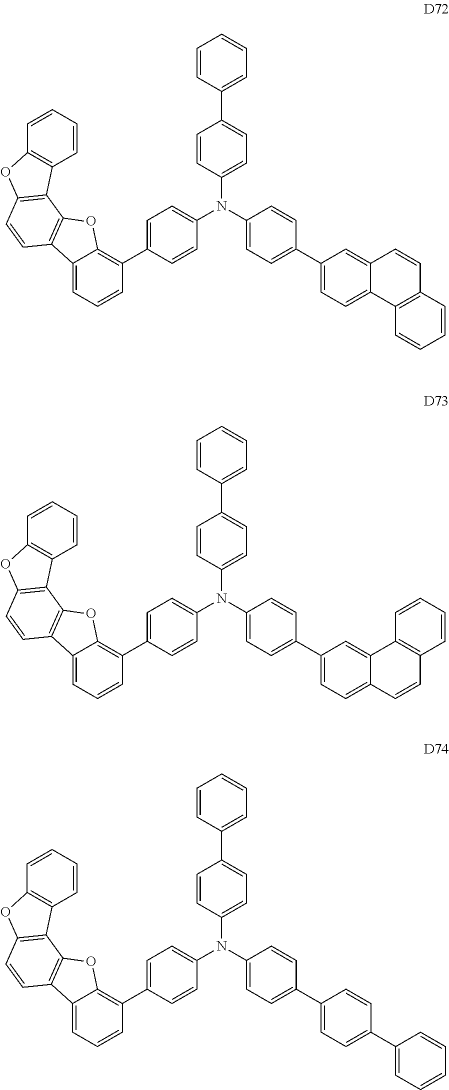 [SMILES: c1ccc(-c2ccc(N(c3ccc(-c4ccc5c(ccc6ccccc65)c4)cc3)c3ccc(-c4cccc5c4oc4c5ccc5oc6ccccc6c54)cc3)cc2)cc1 ; c1ccc(-c2ccc(N(c3ccc(-c4ccc5ccc6ccccc6c5c4)cc3)c3ccc(-c4cccc5c4oc4c5ccc5oc6ccccc6c54)cc3)cc2)cc1 ; c1ccc(-c2ccc(-c3ccc(N(c4ccc(-c5ccccc5)cc4)c4ccc(-c5cccc6c5oc5c6ccc6oc7ccccc7c65)cc4)cc3)cc2)cc1]

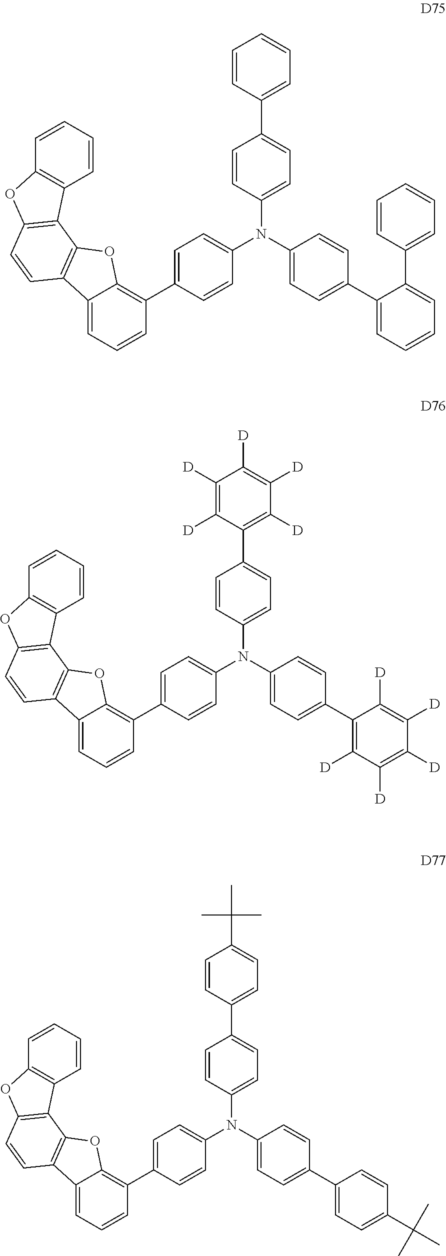 [SMILES: c1ccc(-c2ccc(N(c3ccc(-c4ccccc4-c4ccccc4)cc3)c3ccc(-c4cccc5c4oc4c5ccc5oc6ccccc6c54)cc3)cc2)cc1 ; [2H]c1c([2H])c([2H])c(-c2ccc(N(c3ccc(-c4c([2H])c([2H])c([2H])c([2H])c4[2H])cc3)c3ccc(-c4cccc5c4oc4c5ccc5oc6ccccc6c54)cc3)cc2)c([2H])c1[2H] ; CC(C)(C)c1ccc(-c2ccc(N(c3ccc(-c4ccc(C(C)(C)C)cc4)cc3)c3ccc(-c4cccc5c4oc4c5ccc5oc6ccccc6c54)cc3)cc2)cc1]

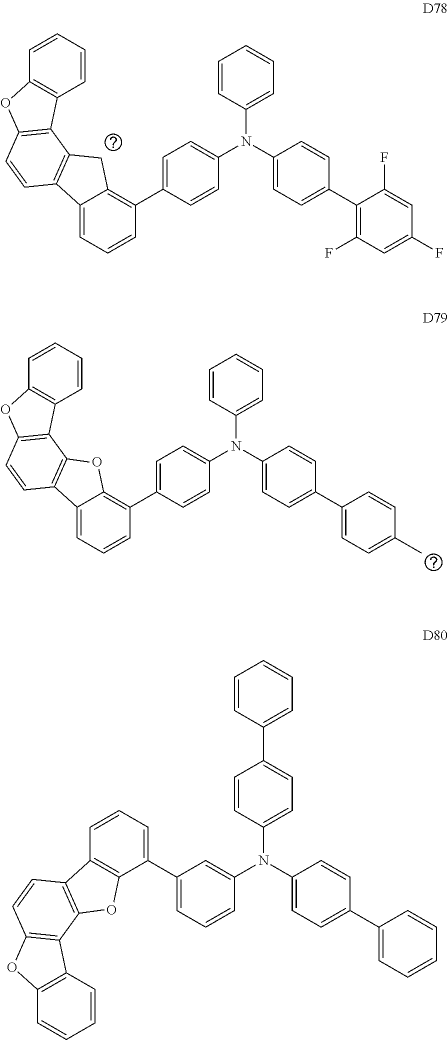 [SMILES: Fc1cc(F)c(-c2ccc(N(c3ccccc3)c3ccc(-c4cccc5c4Cc4c-5ccc5oc6ccccc6c45)cc3)cc2)c(F)c1 ; Cc1ccc(-c2ccc(N(c3ccccc3)c3ccc(-c4cccc5c4oc4c5ccc5oc6ccccc6c54)cc3)cc2)cc1 ; c1ccc(-c2ccc(N(c3ccc(-c4ccccc4)cc3)c3cccc(-c4cccc5c4oc4c5ccc5oc6ccccc6c54)c3)cc2)cc1]

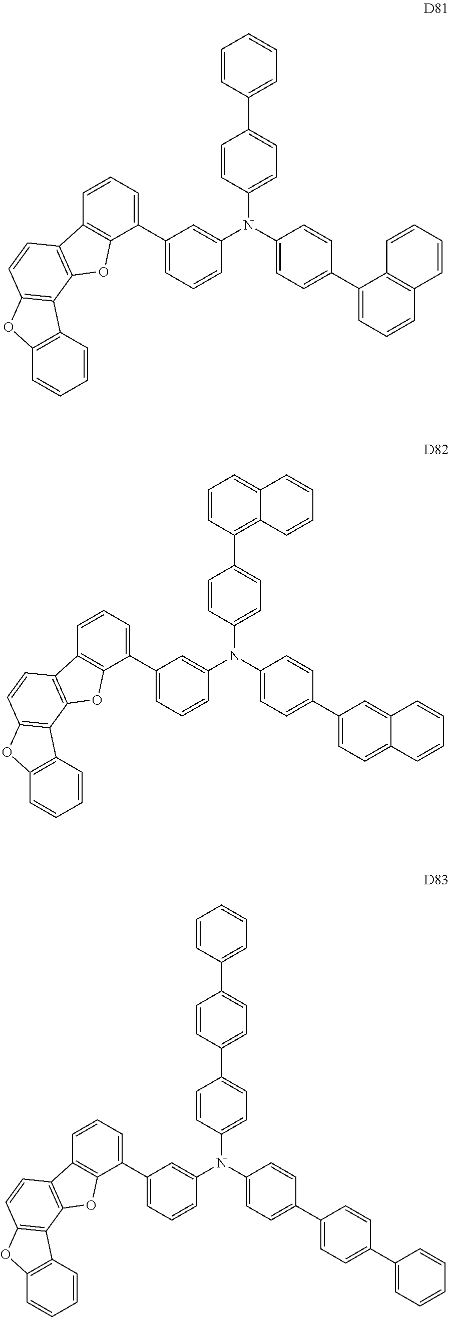 [SMILES: c1ccc(-c2ccc(N(c3ccc(-c4cccc5ccccc45)cc3)c3cccc(-c4cccc5c4oc4c5ccc5oc6ccccc6c54)c3)cc2)cc1 ; c1cc(-c2cccc3c2oc2c3ccc3oc4ccccc4c32)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6c5oc5c6ccc6oc7ccccc7c65)c4)cc3)cc2)cc1]

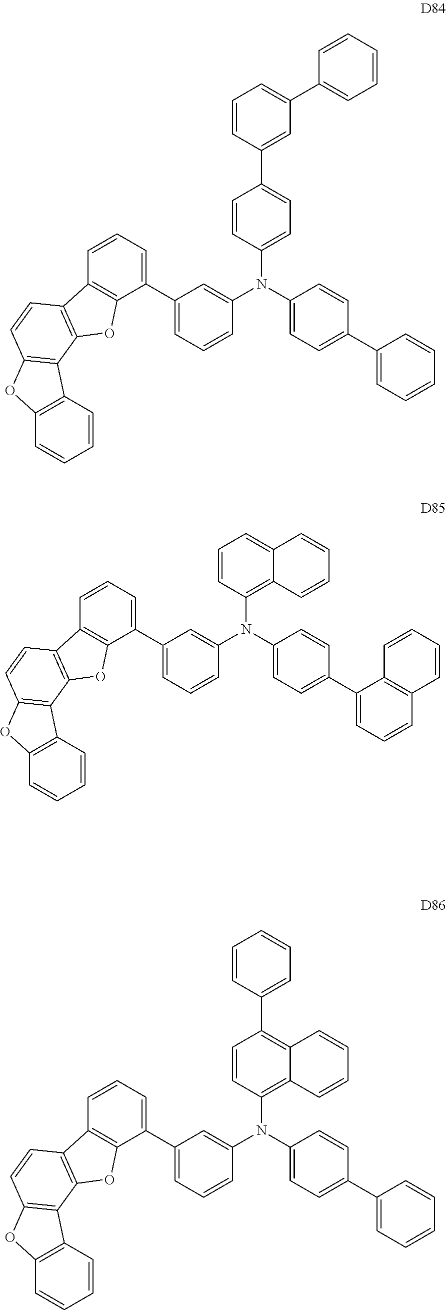 [SMILES: c1ccc(-c2ccc(N(c3ccc(-c4cccc(-c5ccccc5)c4)cc3)c3cccc(-c4cccc5c4oc4c5ccc5oc6ccccc6c54)c3)cc2)cc1 ; c1cc(-c2cccc3c2oc2c3ccc3oc4ccccc4c32)cc(N(c2ccc(-c3cccc4ccccc34)cc2)c2cccc3ccccc23)c1 ; c1ccc(-c2ccc(N(c3cccc(-c4cccc5c4oc4c5ccc5oc6ccccc6c54)c3)c3ccc(-c4ccccc4)c4ccccc34)cc2)cc1]

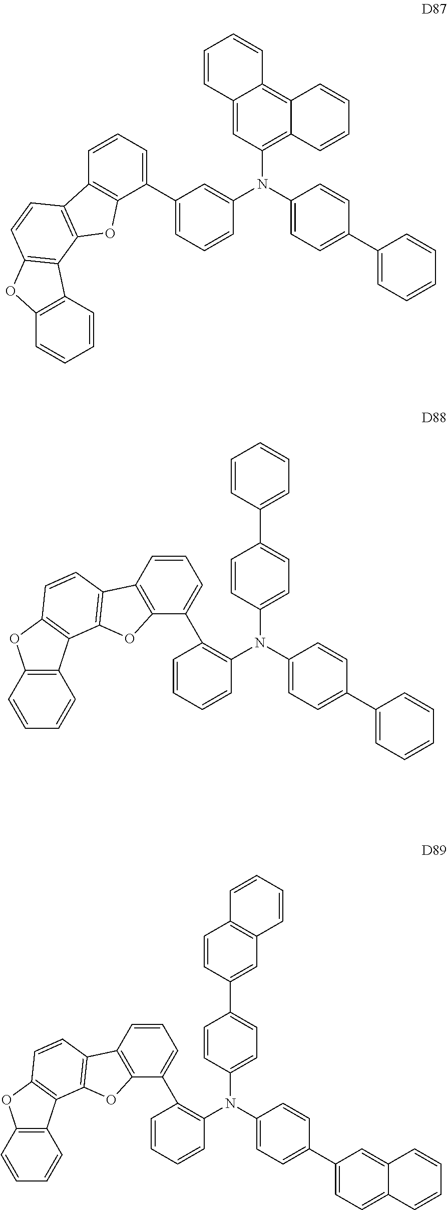 [SMILES: c1ccc(-c2ccc(N(c3cccc(-c4cccc5c4oc4c5ccc5oc6ccccc6c54)c3)c3cc4ccccc4c4ccccc34)cc2)cc1 ; c1ccc(-c2ccc(N(c3ccc(-c4ccccc4)cc3)c3ccccc3-c3cccc4c3oc3c4ccc4oc5ccccc5c43)cc2)cc1 ; c1ccc(N(c2ccc(-c3ccc4ccccc4c3)cc2)c2ccc(-c3ccc4ccccc4c3)cc2)c(-c2cccc3c2oc2c3ccc3oc4ccccc4c32)c1]

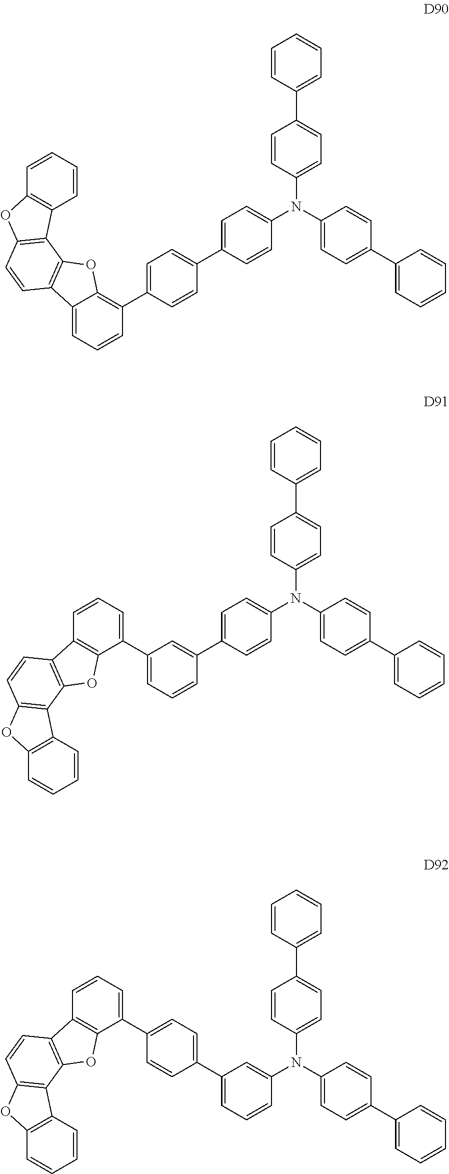 [SMILES: c1ccc(-c2ccc(N(c3ccc(-c4ccccc4)cc3)c3ccc(-c4ccc(-c5cccc6c5oc5c6ccc6oc7ccccc7c65)cc4)cc3)cc2)cc1 ; c1ccc(-c2ccc(N(c3ccc(-c4ccccc4)cc3)c3ccc(-c4cccc(-c5cccc6c5oc5c6ccc6oc7ccccc7c65)c4)cc3)cc2)cc1 ; c1ccc(-c2ccc(N(c3ccc(-c4ccccc4)cc3)c3cccc(-c4ccc(-c5cccc6c5oc5c6ccc6oc7ccccc7c65)cc4)c3)cc2)cc1]

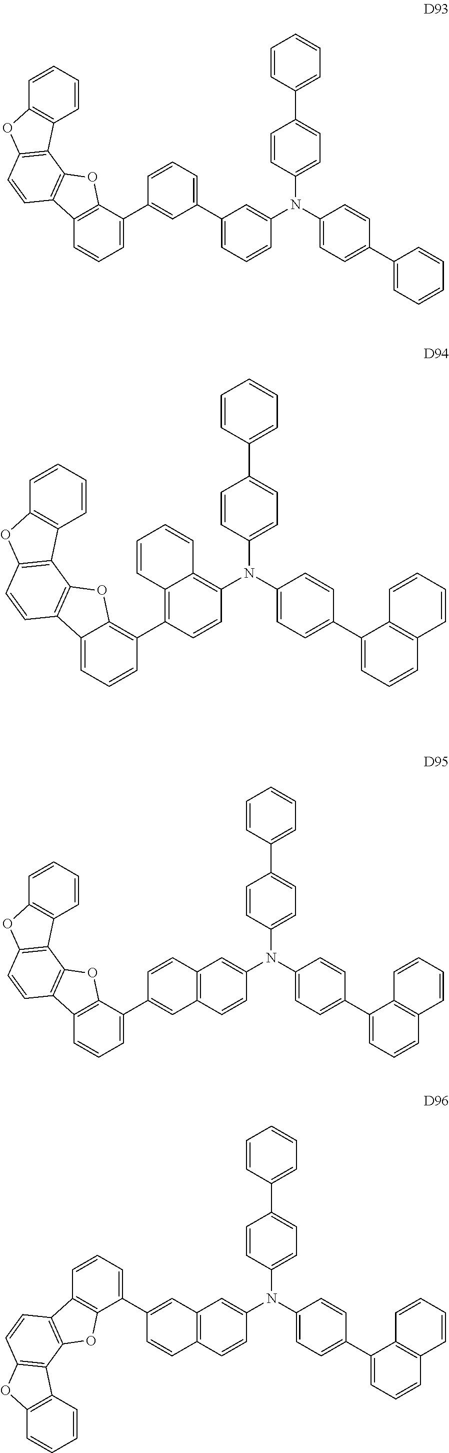 [SMILES: c1ccc(-c2ccc(N(c3ccc(-c4ccccc4)cc3)c3cccc(-c4cccc(-c5cccc6c5oc5c6ccc6oc7ccccc7c65)c4)c3)cc2)cc1 ; c1ccc(-c2ccc(N(c3ccc(-c4cccc5ccccc45)cc3)c3ccc(-c4cccc5c4oc4c5ccc5oc6ccccc6c54)c4ccccc34)cc2)cc1 ; c1ccc(-c2ccc(N(c3ccc(-c4cccc5ccccc45)cc3)c3ccc4cc(-c5cccc6c5oc5c6ccc6oc7ccccc7c65)ccc4c3)cc2)cc1 ; c1ccc(-c2ccc(N(c3ccc(-c4cccc5ccccc45)cc3)c3ccc4ccc(-c5cccc6c5oc5c6ccc6oc7ccccc7c65)cc4c3)cc2)cc1]

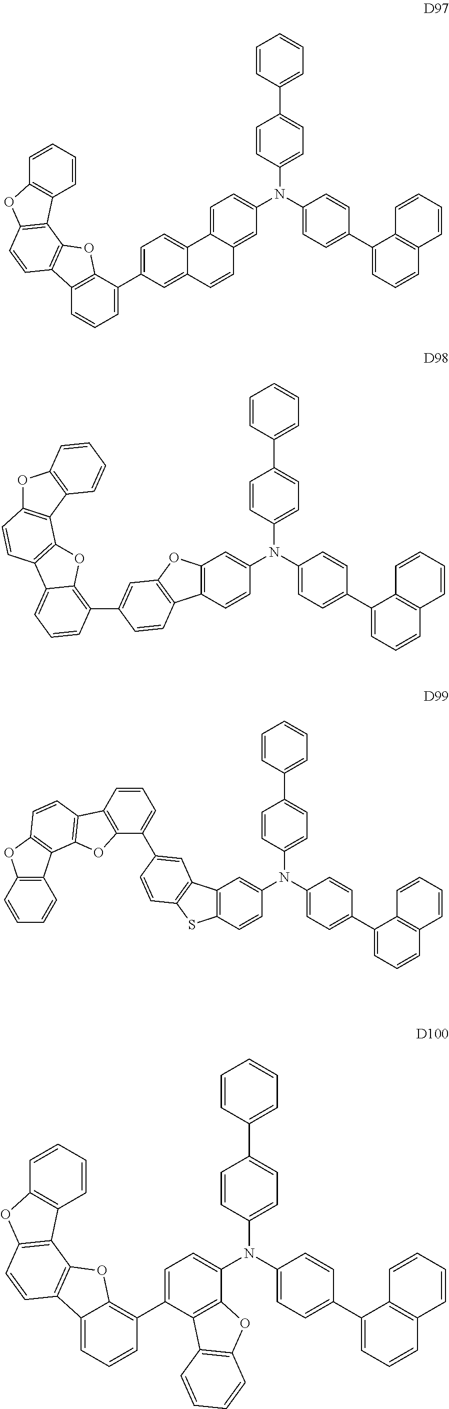 [SMILES: c1ccc(-c2ccc(N(c3ccc(-c4cccc5ccccc45)cc3)c3ccc4c(ccc5cc(-c6cccc7c6oc6c7ccc7oc8ccccc8c76)ccc54)c3)cc2)cc1 ; c1ccc(-c2ccc(N(c3ccc(-c4cccc5ccccc45)cc3)c3ccc4c(c3)oc3cc(-c5cccc6c5oc5c6ccc6oc7ccccc7c65)ccc34)cc2)cc1 ; c1ccc(-c2ccc(N(c3ccc(-c4cccc5ccccc45)cc3)c3ccc4sc5ccc(-c6cccc7c6oc6c7ccc7oc8ccccc8c76)cc5c4c3)cc2)cc1 ; c1ccc(-c2ccc(N(c3ccc(-c4cccc5ccccc45)cc3)c3ccc(-c4cccc5c4oc4c5ccc5oc6ccccc6c54)c4c3oc3ccccc34)cc2)cc1]

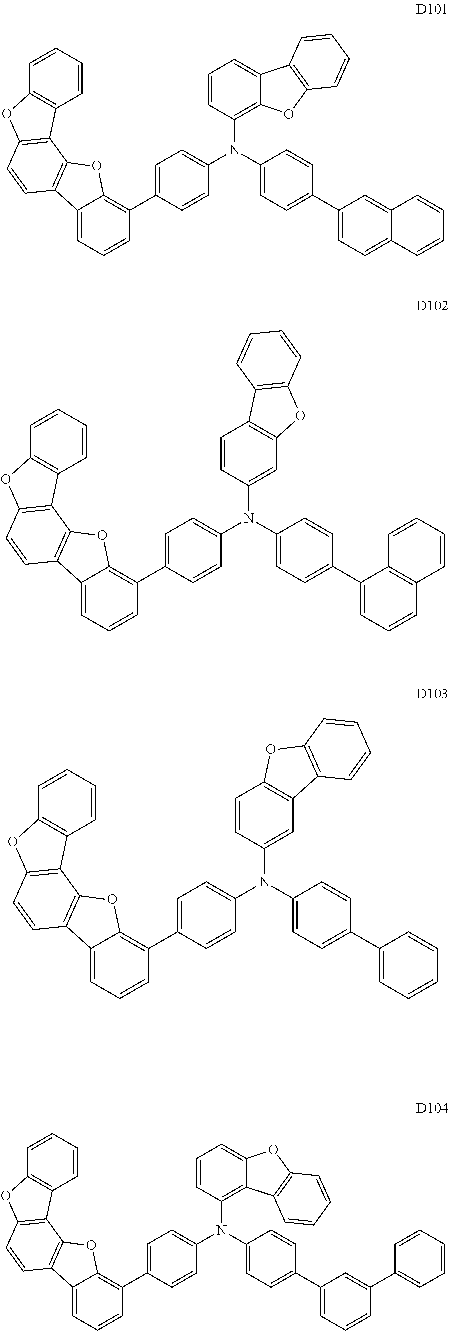 [SMILES: c1ccc2cc(-c3ccc(N(c4ccc(-c5cccc6c5oc5c6ccc6oc7ccccc7c65)cc4)c4cccc5c4oc4ccccc45)cc3)ccc2c1 ; c1ccc2c(-c3ccc(N(c4ccc(-c5cccc6c5oc5c6ccc6oc7ccccc7c65)cc4)c4ccc5c(c4)oc4ccccc45)cc3)cccc2c1 ; c1ccc(-c2ccc(N(c3ccc(-c4cccc5c4oc4c5ccc5oc6ccccc6c54)cc3)c3ccc4oc5ccccc5c4c3)cc2)cc1 ; c1ccc(-c2cccc(-c3ccc(N(c4ccc(-c5cccc6c5oc5c6ccc6oc7ccccc7c65)cc4)c4cccc5oc6ccccc6c45)cc3)c2)cc1]

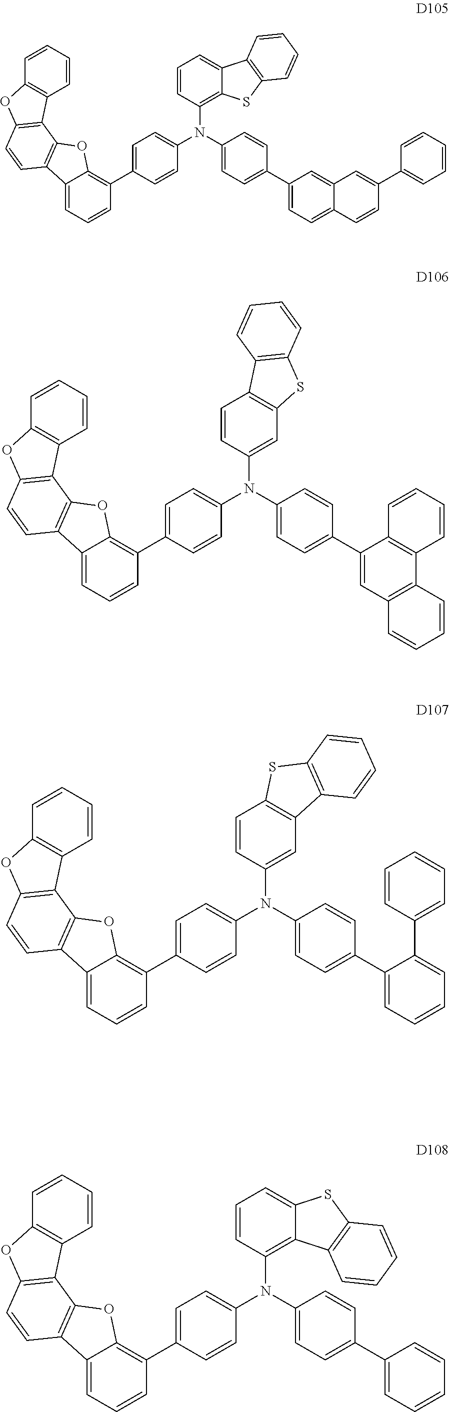 [SMILES: c1ccc(-c2ccc3ccc(-c4ccc(N(c5ccc(-c6cccc7c6oc6c7ccc7oc8ccccc8c76)cc5)c5cccc6c5sc5ccccc56)cc4)cc3c2)cc1 ; c1ccc2c(c1)cc(-c1ccc(N(c3ccc(-c4cccc5c4oc4c5ccc5oc6ccccc6c54)cc3)c3ccc4c(c3)sc3ccccc34)cc1)c1ccccc12 ; c1ccc(-c2ccccc2-c2ccc(N(c3ccc(-c4cccc5c4oc4c5ccc5oc6ccccc6c54)cc3)c3ccc4sc5ccccc5c4c3)cc2)cc1 ; c1ccc(-c2ccc(N(c3ccc(-c4cccc5c4oc4c5ccc5oc6ccccc6c54)cc3)c3cccc4sc5ccccc5c34)cc2)cc1]

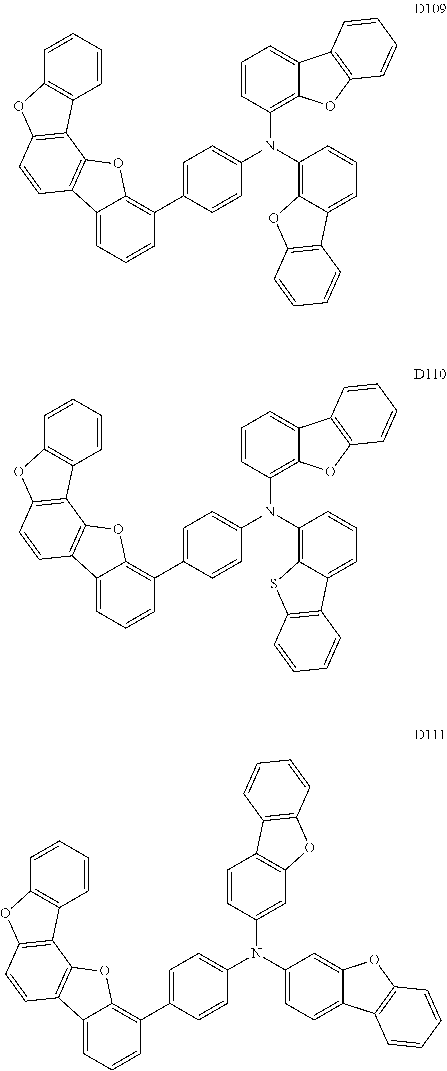 [SMILES: c1ccc2c(c1)oc1c(N(c3ccc(-c4cccc5c4oc4c5ccc5oc6ccccc6c54)cc3)c3cccc4c3oc3ccccc34)cccc12 ; c1ccc2c(c1)oc1c(N(c3ccc(-c4cccc5c4oc4c5ccc5oc6ccccc6c54)cc3)c3cccc4c3sc3ccccc34)cccc12 ; c1ccc2c(c1)oc1cc(N(c3ccc(-c4cccc5c4oc4c5ccc5oc6ccccc6c54)cc3)c3ccc4c(c3)oc3ccccc34)ccc12]

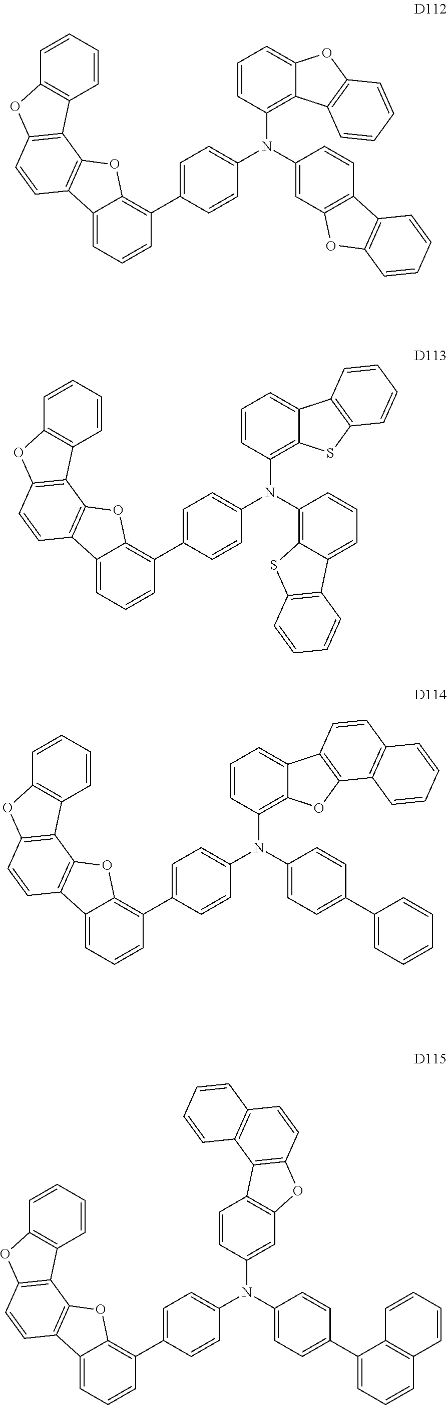 [SMILES: c1ccc2c(c1)oc1cc(N(c3ccc(-c4cccc5c4oc4c5ccc5oc6ccccc6c54)cc3)c3cccc4oc5ccccc5c34)ccc12 ; c1ccc2c(c1)oc1ccc3c4cccc(-c5ccc(N(c6cccc7c6sc6ccccc67)c6cccc7c6sc6ccccc67)cc5)c4oc3c12 ; c1ccc(-c2ccc(N(c3ccc(-c4cccc5c4oc4c5ccc5oc6ccccc6c54)cc3)c3cccc4c3oc3c5ccccc5ccc43)cc2)cc1 ; c1ccc2c(-c3ccc(N(c4ccc(-c5cccc6c5oc5c6ccc6oc7ccccc7c65)cc4)c4ccc5c(c4)oc4ccc6ccccc6c45)cc3)cccc2c1]

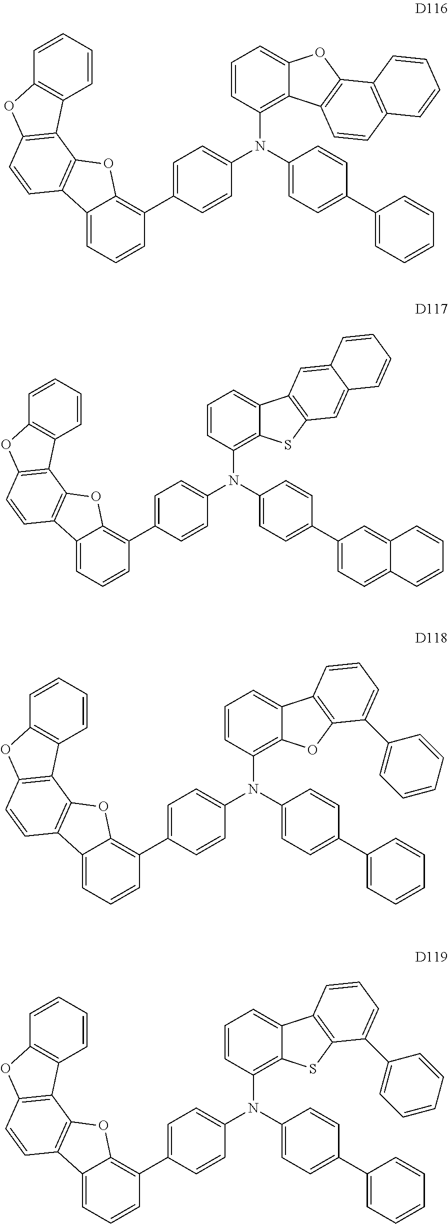 [SMILES: c1ccc(-c2ccc(N(c3ccc(-c4cccc5c4oc4c5ccc5oc6ccccc6c54)cc3)c3cccc4oc5c6ccccc6ccc5c34)cc2)cc1 ; c1ccc2cc(-c3ccc(N(c4ccc(-c5cccc6c5oc5c6ccc6oc7ccccc7c65)cc4)c4cccc5c4sc4cc6ccccc6cc45)cc3)ccc2c1 ; c1ccc(-c2ccc(N(c3ccc(-c4cccc5c4oc4c5ccc5oc6ccccc6c54)cc3)c3cccc4c3oc3c(-c5ccccc5)cccc34)cc2)cc1 ; c1ccc(-c2ccc(N(c3ccc(-c4cccc5c4oc4c5ccc5oc6ccccc6c54)cc3)c3cccc4c3sc3c(-c5ccccc5)cccc34)cc2)cc1]

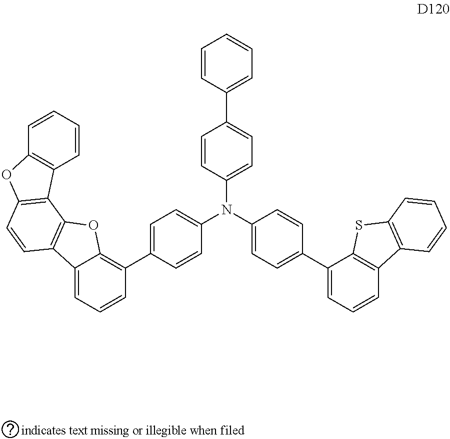 [SMILES: c1ccc(-c2ccc(N(c3ccc(-c4cccc5c4oc4c5ccc5oc6ccccc6c54)cc3)c3ccc(-c4cccc5c4sc4ccccc45)cc3)cc2)cc1]

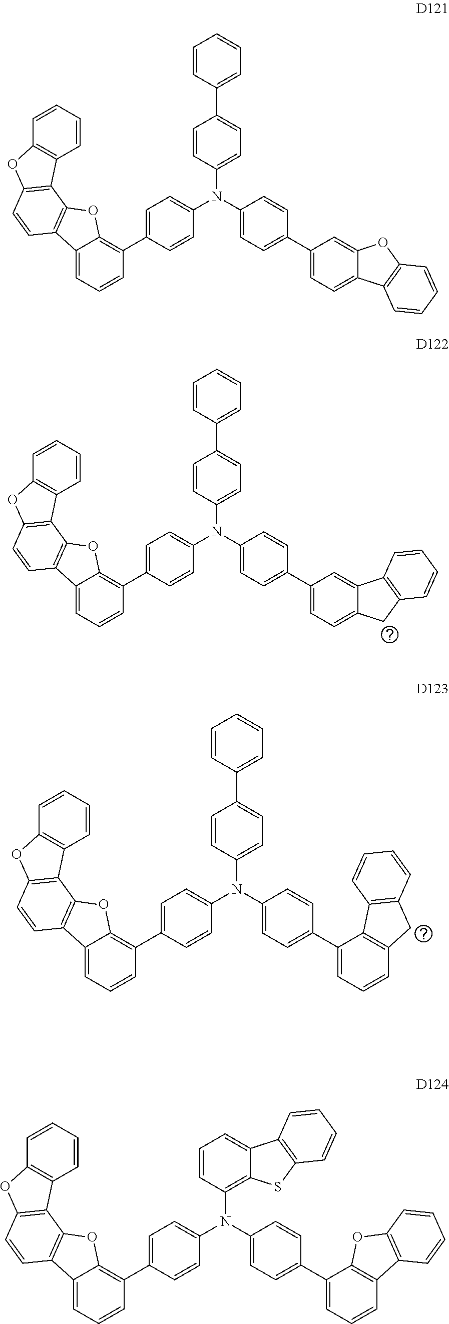 [SMILES: c1ccc(-c2ccc(N(c3ccc(-c4ccc5c(c4)oc4ccccc45)cc3)c3ccc(-c4cccc5c4oc4c5ccc5oc6ccccc6c54)cc3)cc2)cc1 ; c1ccc(-c2ccc(N(c3ccc(-c4ccc5c(c4)-c4ccccc4C5)cc3)c3ccc(-c4cccc5c4oc4c5ccc5oc6ccccc6c54)cc3)cc2)cc1 ; c1ccc(-c2ccc(N(c3ccc(-c4cccc5c4-c4ccccc4C5)cc3)c3ccc(-c4cccc5c4oc4c5ccc5oc6ccccc6c54)cc3)cc2)cc1 ; c1ccc2c(c1)oc1c(-c3ccc(N(c4ccc(-c5cccc6c5oc5c6ccc6oc7ccccc7c65)cc4)c4cccc5c4sc4ccccc45)cc3)cccc12]

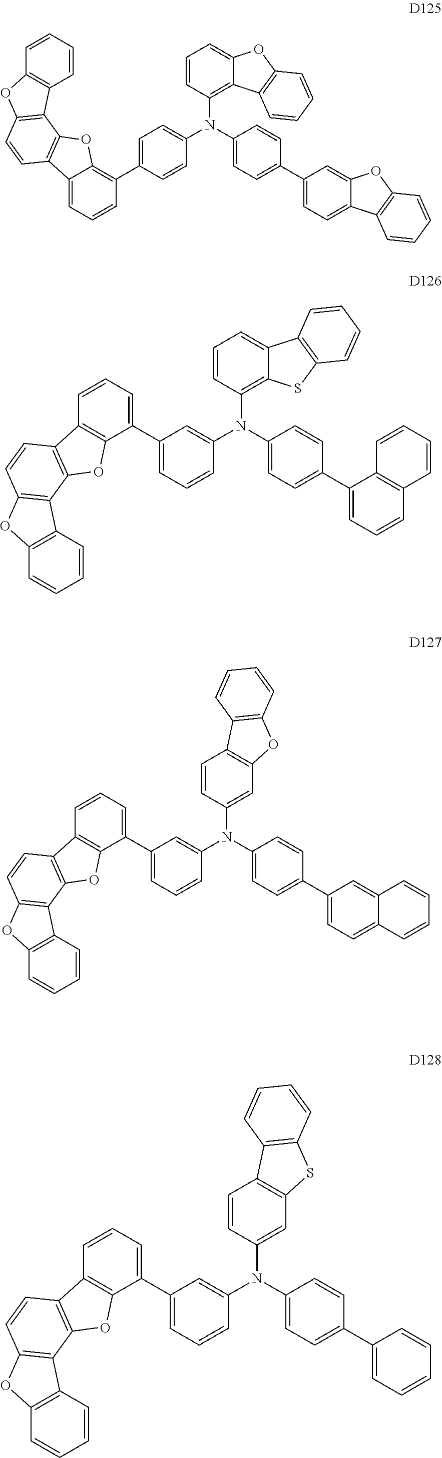 [SMILES: c1ccc2c(c1)oc1cc(-c3ccc(N(c4ccc(-c5cccc6c5oc5c6ccc6oc7ccccc7c65)cc4)c4cccc5oc6ccccc6c45)cc3)ccc12 ; c1cc(-c2cccc3c2oc2c3ccc3oc4ccccc4c32)cc(N(c2ccc(-c3cccc4ccccc34)cc2)c2cccc3c2sc2ccccc23)c1 ; c1cc(-c2cccc3c2oc2c3ccc3oc4ccccc4c32)cc(N(c2ccc(-c3ccc4ccccc4c3)cc2)c2ccc3c(c2)oc2ccccc23)c1 ; c1ccc(-c2ccc(N(c3cccc(-c4cccc5c4oc4c5ccc5oc6ccccc6c54)c3)c3ccc4c(c3)sc3ccccc34)cc2)cc1]

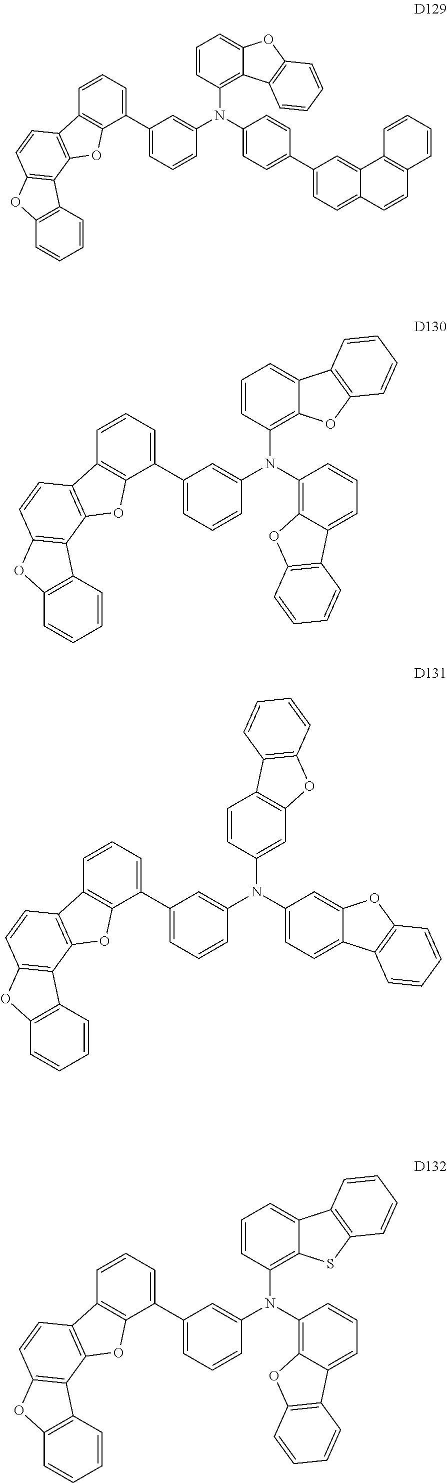 [SMILES: c1cc(-c2cccc3c2oc2c3ccc3oc4ccccc4c32)cc(N(c2ccc(-c3ccc4ccc5ccccc5c4c3)cc2)c2cccc3oc4ccccc4c23)c1 ; c1cc(-c2cccc3c2oc2c3ccc3oc4ccccc4c32)cc(N(c2cccc3c2oc2ccccc23)c2cccc3c2oc2ccccc23)c1 ; c1cc(-c2cccc3c2oc2c3ccc3oc4ccccc4c32)cc(N(c2ccc3c(c2)oc2ccccc23)c2ccc3c(c2)oc2ccccc23)c1 ; c1cc(-c2cccc3c2oc2c3ccc3oc4ccccc4c32)cc(N(c2cccc3c2oc2ccccc23)c2cccc3c2sc2ccccc23)c1]

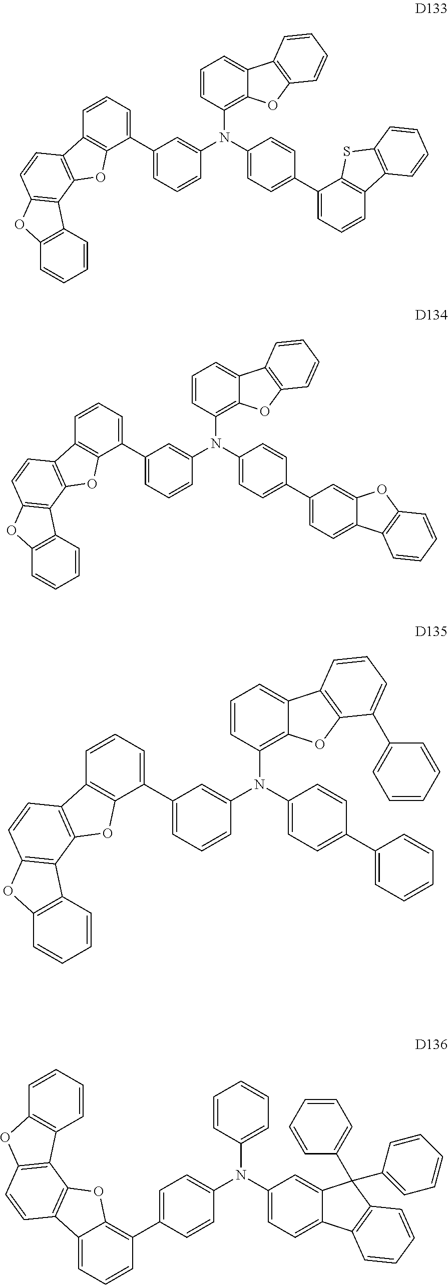 [SMILES: c1cc(-c2cccc3c2oc2c3ccc3oc4ccccc4c32)cc(N(c2ccc(-c3cccc4c3sc3ccccc34)cc2)c2cccc3c2oc2ccccc23)c1 ; c1cc(-c2cccc3c2oc2c3ccc3oc4ccccc4c32)cc(N(c2ccc(-c3ccc4c(c3)oc3ccccc34)cc2)c2cccc3c2oc2ccccc23)c1 ; c1ccc(-c2ccc(N(c3cccc(-c4cccc5c4oc4c5ccc5oc6ccccc6c54)c3)c3cccc4c3oc3c(-c5ccccc5)cccc34)cc2)cc1 ; c1ccc(N(c2ccc(-c3cccc4c3oc3c4ccc4oc5ccccc5c43)cc2)c2ccc3c(c2)C(c2ccccc2)(c2ccccc2)c2ccccc2-3)cc1]

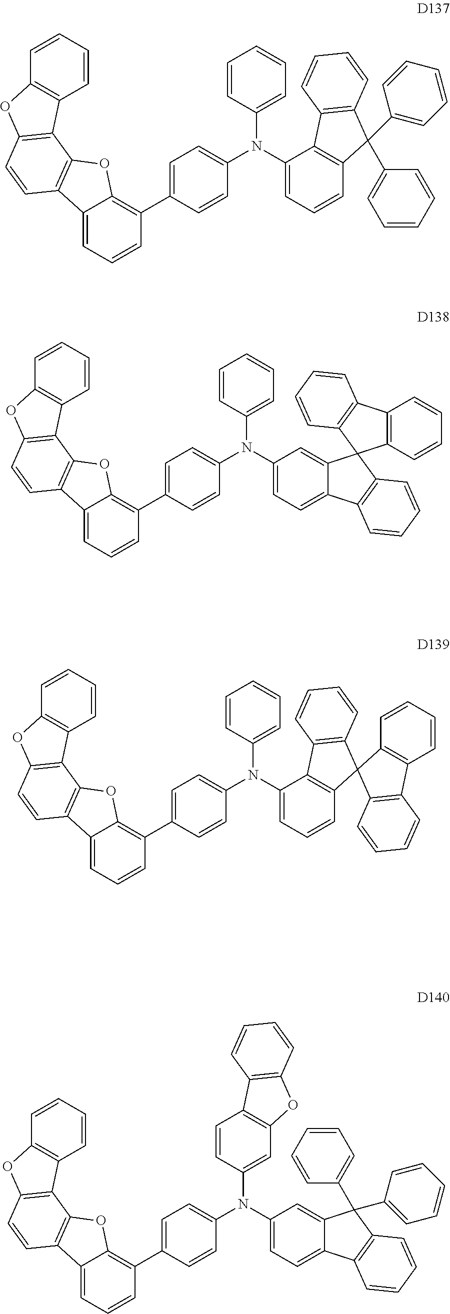 [SMILES: c1ccc(N(c2ccc(-c3cccc4c3oc3c4ccc4oc5ccccc5c43)cc2)c2cccc3c2-c2ccccc2C3(c2ccccc2)c2ccccc2)cc1 ; c1ccc(N(c2ccc(-c3cccc4c3oc3c4ccc4oc5ccccc5c43)cc2)c2ccc3c(c2)C2(c4ccccc4-c4ccccc42)c2ccccc2-3)cc1 ; c1ccc(N(c2ccc(-c3cccc4c3oc3c4ccc4oc5ccccc5c43)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6c5oc5c6ccc6oc7ccccc7c65)cc4)c4ccc5c(c4)oc4ccccc45)cc32)cc1]

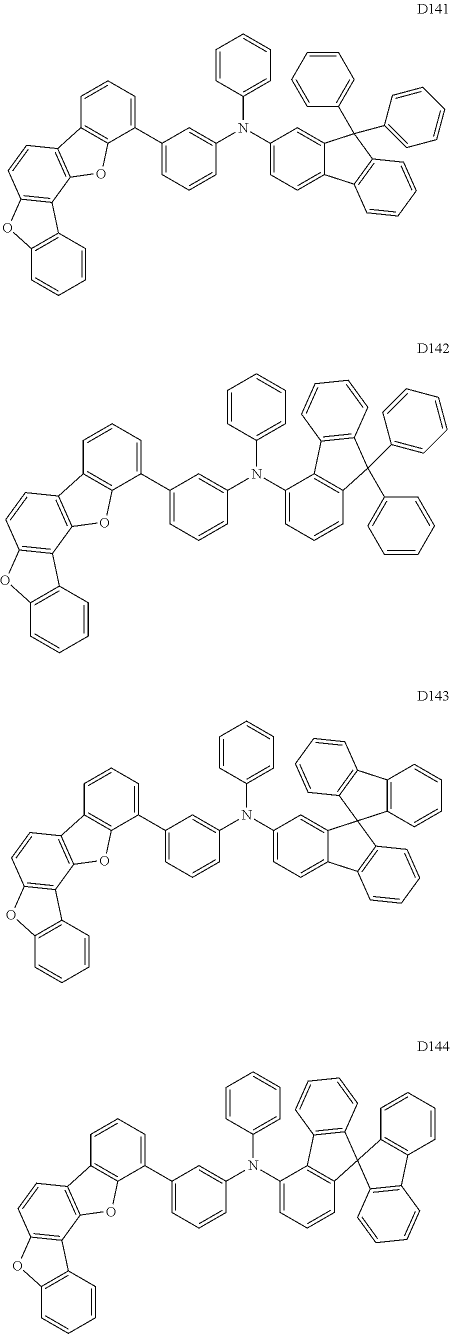 [SMILES: c1ccc(N(c2cccc(-c3cccc4c3oc3c4ccc4oc5ccccc5c43)c2)c2ccc3c(c2)C(c2ccccc2)(c2ccccc2)c2ccccc2-3)cc1 ; c1ccc(N(c2cccc(-c3cccc4c3oc3c4ccc4oc5ccccc5c43)c2)c2cccc3c2-c2ccccc2C3(c2ccccc2)c2ccccc2)cc1 ; c1ccc(N(c2cccc(-c3cccc4c3oc3c4ccc4oc5ccccc5c43)c2)c2ccc3c(c2)C2(c4ccccc4-c4ccccc42)c2ccccc2-3)cc1 ; c1ccc(N(c2cccc(-c3cccc4c3oc3c4ccc4oc5ccccc5c43)c2)c2cccc3c2-c2ccccc2C32c3ccccc3-c3ccccc32)cc1]

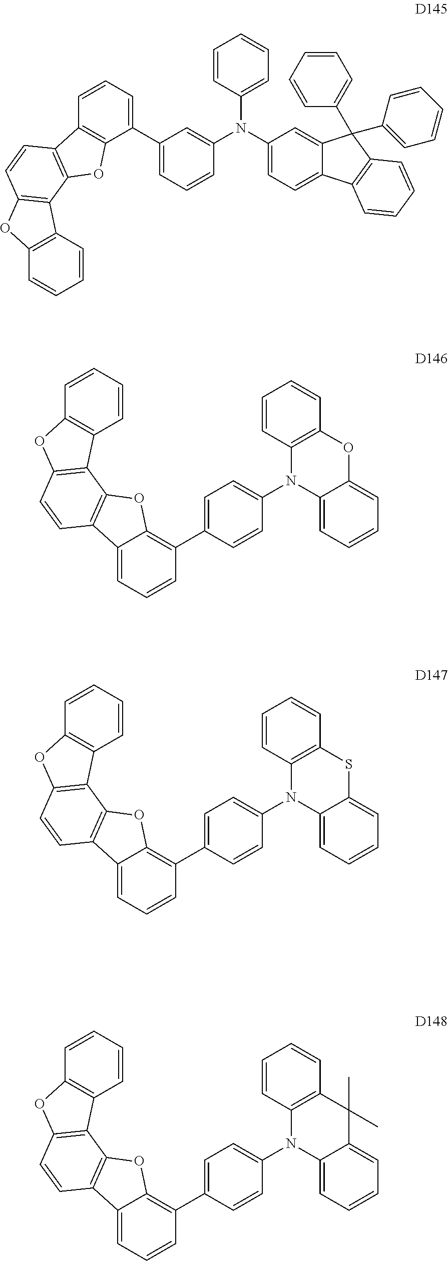 [SMILES: c1ccc(N(c2cccc(-c3cccc4c3oc3c4ccc4oc5ccccc5c43)c2)c2ccc3c(c2)C(c2ccccc2)(c2ccccc2)c2ccccc2-3)cc1 ; c1ccc2c(c1)Oc1ccccc1N2c1ccc(-c2cccc3c2oc2c3ccc3oc4ccccc4c32)cc1 ; c1ccc2c(c1)Sc1ccccc1N2c1ccc(-c2cccc3c2oc2c3ccc3oc4ccccc4c32)cc1 ; CC1(C)c2ccccc2N(c2ccc(-c3cccc4c3oc3c4ccc4oc5ccccc5c43)cc2)c2ccccc21]

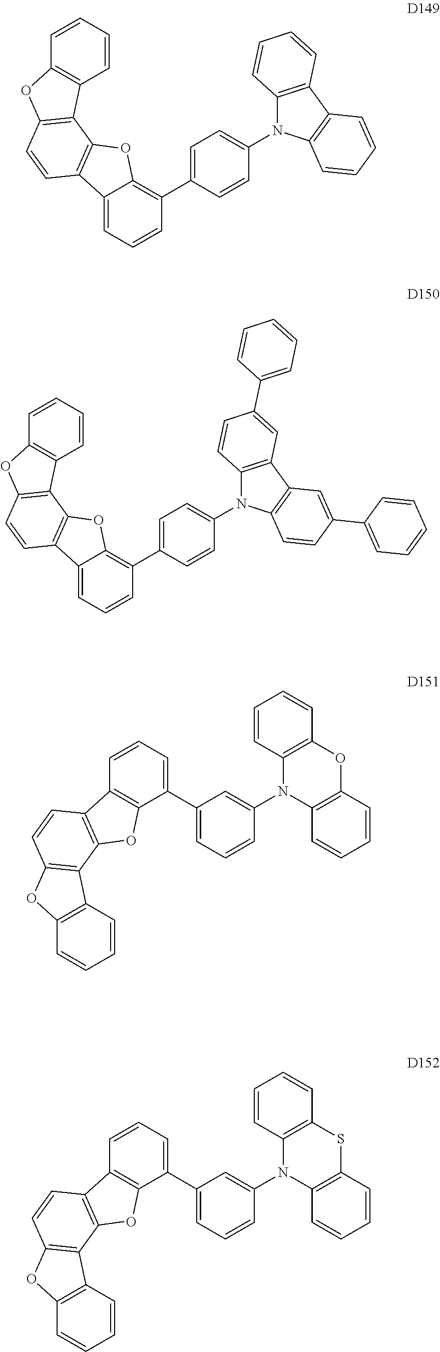 [SMILES: c1ccc2c(c1)oc1ccc3c4cccc(-c5ccc(-n6c7ccccc7c7ccccc76)cc5)c4oc3c12 ; c1ccc(-c2ccc3c(c2)c2cc(-c4ccccc4)ccc2n3-c2ccc(-c3cccc4c3oc3c4ccc4oc5ccccc5c43)cc2)cc1 ; c1cc(-c2cccc3c2oc2c3ccc3oc4ccccc4c32)cc(N2c3ccccc3Oc3ccccc32)c1 ; c1cc(-c2cccc3c2oc2c3ccc3oc4ccccc4c32)cc(N2c3ccccc3Sc3ccccc32)c1]

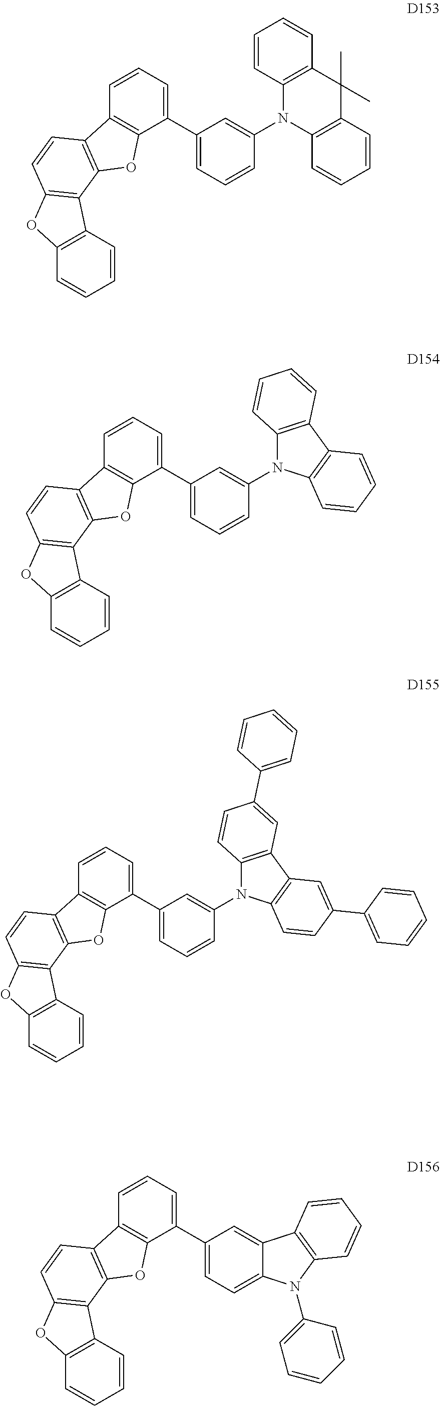 [SMILES: CC1(C)c2ccccc2N(c2cccc(-c3cccc4c3oc3c4ccc4oc5ccccc5c43)c2)c2ccccc21 ; c1cc(-c2cccc3c2oc2c3ccc3oc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3oc3c4ccc4oc5ccccc5c43)c2)cc1 ; c1ccc(-n2c3ccccc3c3cc(-c4cccc5c4oc4c5ccc5oc6ccccc6c54)ccc32)cc1]

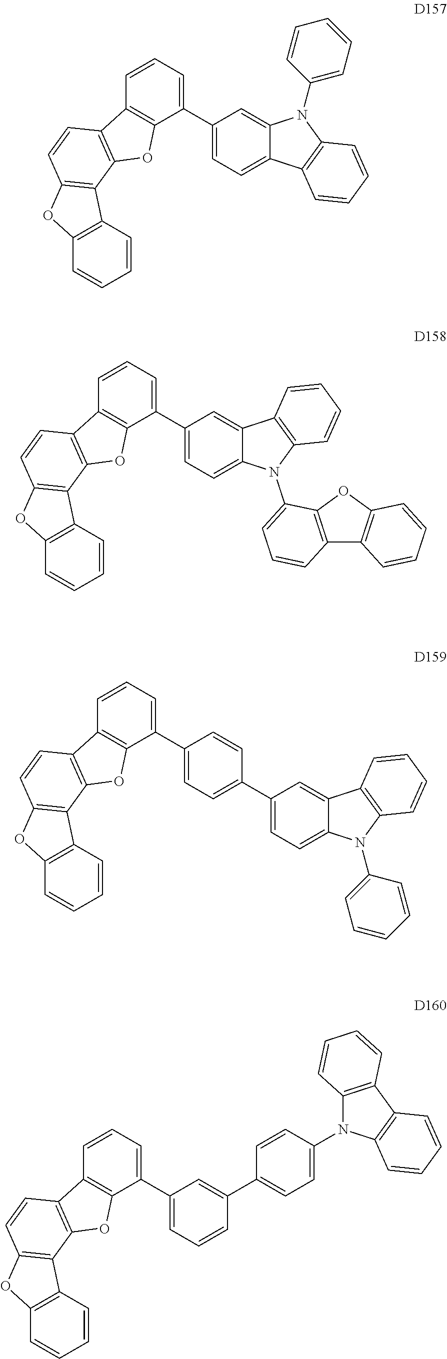 [SMILES: c1ccc(-n2c3ccccc3c3ccc(-c4cccc5c4oc4c5ccc5oc6ccccc6c54)cc32)cc1 ; c1ccc2c(c1)oc1c(-n3c4ccccc4c4cc(-c5cccc6c5oc5c6ccc6oc7ccccc7c65)ccc43)cccc12 ; c1ccc(-n2c3ccccc3c3cc(-c4ccc(-c5cccc6c5oc5c6ccc6oc7ccccc7c65)cc4)ccc32)cc1 ; c1cc(-c2ccc(-n3c4ccccc4c4ccccc43)cc2)cc(-c2cccc3c2oc2c3ccc3oc4ccccc4c32)c1]

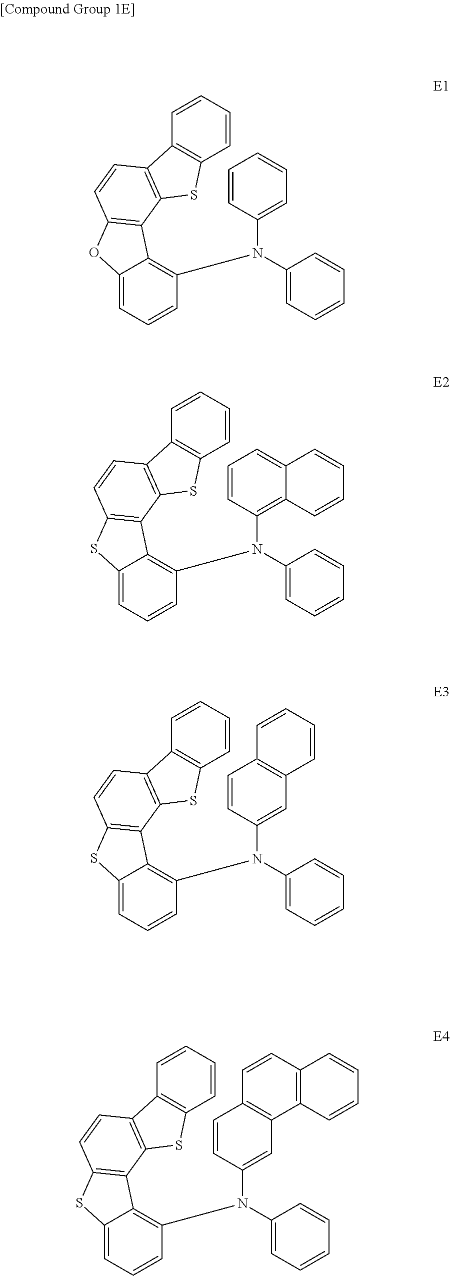 [SMILES: c1ccc(N(c2ccccc2)c2cccc3oc4ccc5c6ccccc6sc5c4c23)cc1 ; c1ccc(N(c2cccc3ccccc23)c2cccc3sc4ccc5c6ccccc6sc5c4c23)cc1 ; c1ccc(N(c2ccc3ccccc3c2)c2cccc3sc4ccc5c6ccccc6sc5c4c23)cc1 ; c1ccc(N(c2ccc3ccc4ccccc4c3c2)c2cccc3sc4ccc5c6ccccc6sc5c4c23)cc1]

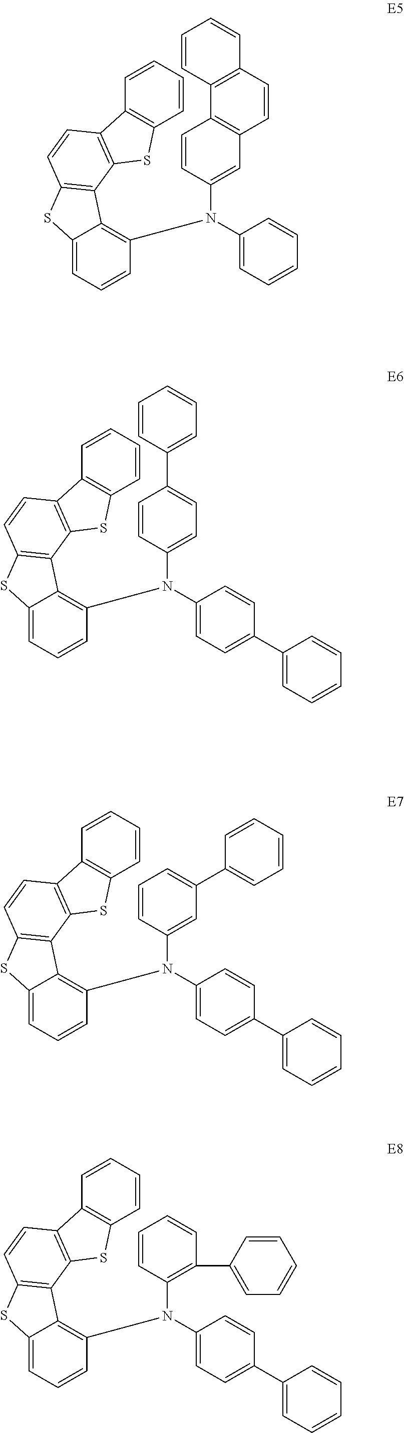 [SMILES: c1ccc(N(c2ccc3c(ccc4ccccc43)c2)c2cccc3sc4ccc5c6ccccc6sc5c4c23)cc1 ; c1ccc(-c2ccc(N(c3ccc(-c4ccccc4)cc3)c3cccc4sc5ccc6c7ccccc7sc6c5c34)cc2)cc1 ; c1ccc(-c2ccc(N(c3cccc(-c4ccccc4)c3)c3cccc4sc5ccc6c7ccccc7sc6c5c34)cc2)cc1 ; c1ccc(-c2ccc(N(c3ccccc3-c3ccccc3)c3cccc4sc5ccc6c7ccccc7sc6c5c34)cc2)cc1]

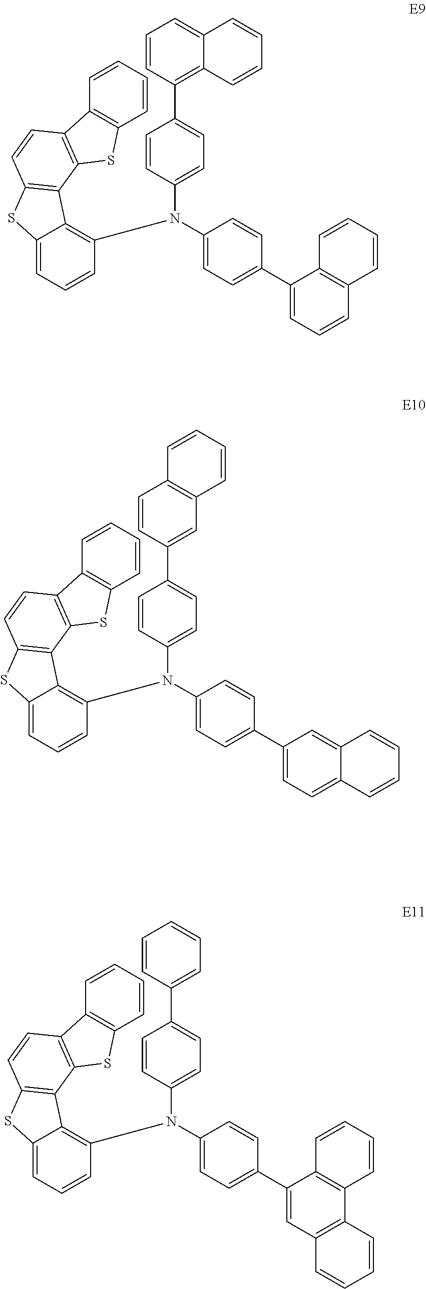 [SMILES: c1ccc2c(-c3ccc(N(c4ccc(-c5cccc6ccccc56)cc4)c4cccc5sc6ccc7c8ccccc8sc7c6c45)cc3)cccc2c1 ; c1ccc2cc(-c3ccc(N(c4ccc(-c5ccc6ccccc6c5)cc4)c4cccc5sc6ccc7c8ccccc8sc7c6c45)cc3)ccc2c1 ; c1ccc(-c2ccc(N(c3ccc(-c4cc5ccccc5c5ccccc45)cc3)c3cccc4sc5ccc6c7ccccc7sc6c5c34)cc2)cc1]

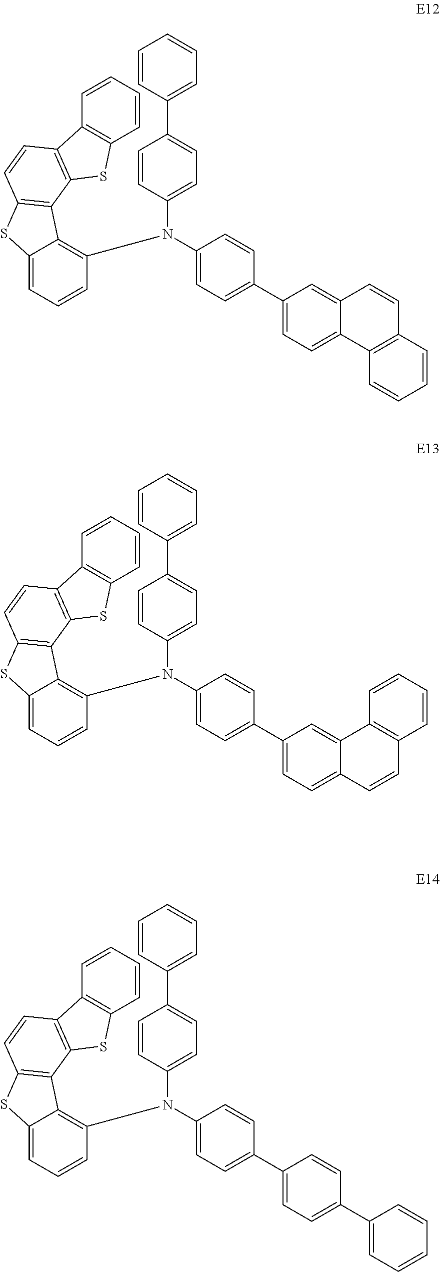 [SMILES: c1ccc(-c2ccc(N(c3ccc(-c4ccc5c(ccc6ccccc65)c4)cc3)c3cccc4sc5ccc6c7ccccc7sc6c5c34)cc2)cc1 ; c1ccc(-c2ccc(N(c3ccc(-c4ccc5ccc6ccccc6c5c4)cc3)c3cccc4sc5ccc6c7ccccc7sc6c5c34)cc2)cc1 ; c1ccc(-c2ccc(-c3ccc(N(c4ccc(-c5ccccc5)cc4)c4cccc5sc6ccc7c8ccccc8sc7c6c45)cc3)cc2)cc1]

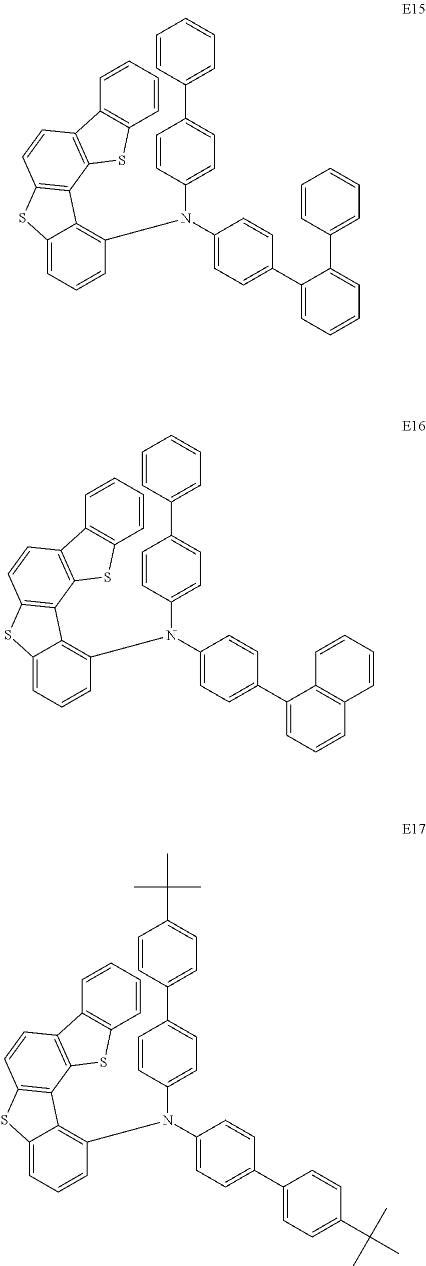 [SMILES: c1ccc(-c2ccc(N(c3ccc(-c4ccccc4-c4ccccc4)cc3)c3cccc4sc5ccc6c7ccccc7sc6c5c34)cc2)cc1 ; c1ccc(-c2ccc(N(c3ccc(-c4cccc5ccccc45)cc3)c3cccc4sc5ccc6c7ccccc7sc6c5c34)cc2)cc1 ; CC(C)(C)c1ccc(-c2ccc(N(c3ccc(-c4ccc(C(C)(C)C)cc4)cc3)c3cccc4sc5ccc6c7ccccc7sc6c5c34)cc2)cc1]

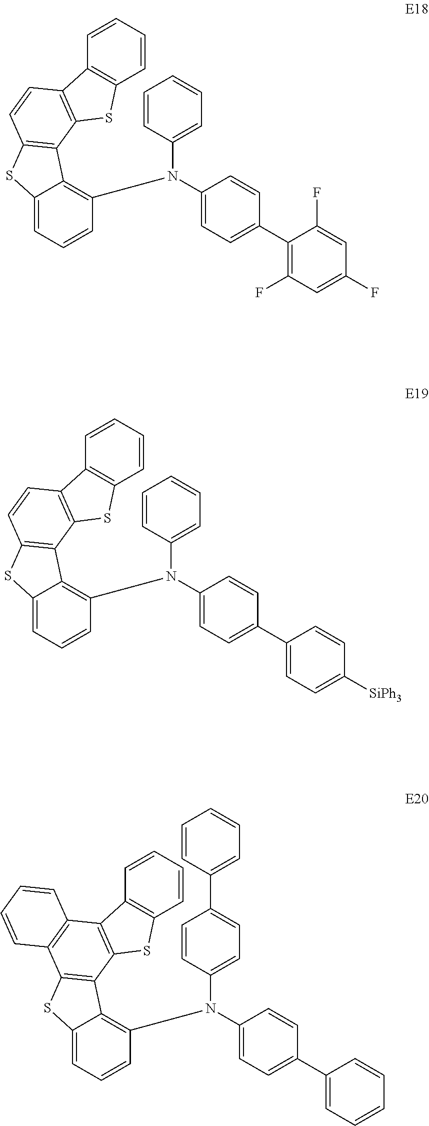

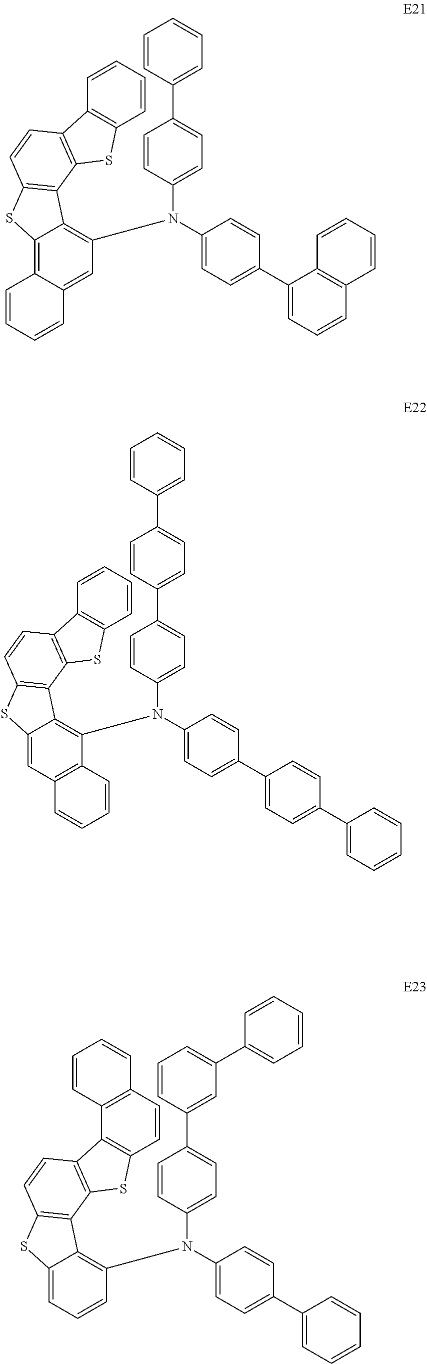 [SMILES: c1ccc(-c2ccc(N(c3ccc(-c4cccc5ccccc45)cc3)c3cc4ccccc4c4sc5ccc6c7ccccc7sc6c5c34)cc2)cc1 ; c1ccc(-c2ccc(-c3ccc(N(c4ccc(-c5ccc(-c6ccccc6)cc5)cc4)c4c5ccccc5cc5sc6ccc7c8ccccc8sc7c6c45)cc3)cc2)cc1 ; c1ccc(-c2ccc(N(c3ccc(-c4cccc(-c5ccccc5)c4)cc3)c3cccc4sc5ccc6c(sc7ccc8ccccc8c76)c5c34)cc2)cc1]

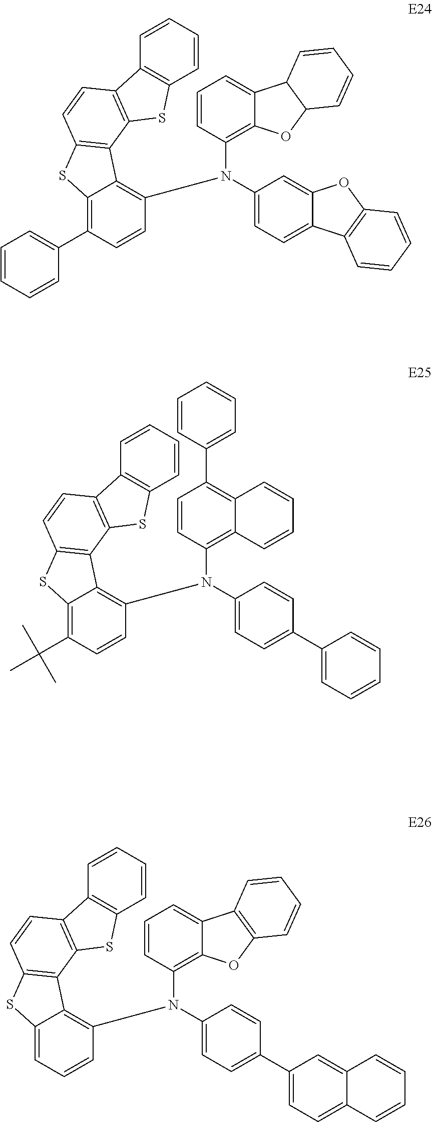 [SMILES: C1=CC2Oc3c(cccc3N(c3ccc4c(c3)oc3ccccc34)c3ccc(-c4ccccc4)c4sc5ccc6c7ccccc7sc6c5c34)C2C=C1 ; CC(C)(C)c1ccc(N(c2ccc(-c3ccccc3)cc2)c2ccc(-c3ccccc3)c3ccccc23)c2c1sc1ccc3c4ccccc4sc3c12 ; c1ccc2cc(-c3ccc(N(c4cccc5c4oc4ccccc45)c4cccc5sc6ccc7c8ccccc8sc7c6c45)cc3)ccc2c1]

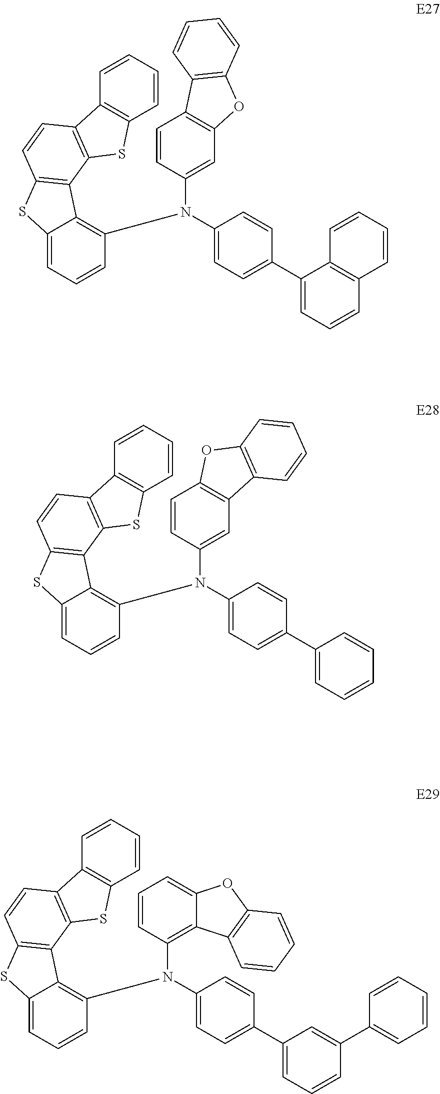 [SMILES: c1ccc2c(-c3ccc(N(c4ccc5c(c4)oc4ccccc45)c4cccc5sc6ccc7c8ccccc8sc7c6c45)cc3)cccc2c1 ; c1ccc(-c2ccc(N(c3ccc4oc5ccccc5c4c3)c3cccc4sc5ccc6c7ccccc7sc6c5c34)cc2)cc1 ; c1ccc(-c2cccc(-c3ccc(N(c4cccc5oc6ccccc6c45)c4cccc5sc6ccc7c8ccccc8sc7c6c45)cc3)c2)cc1]

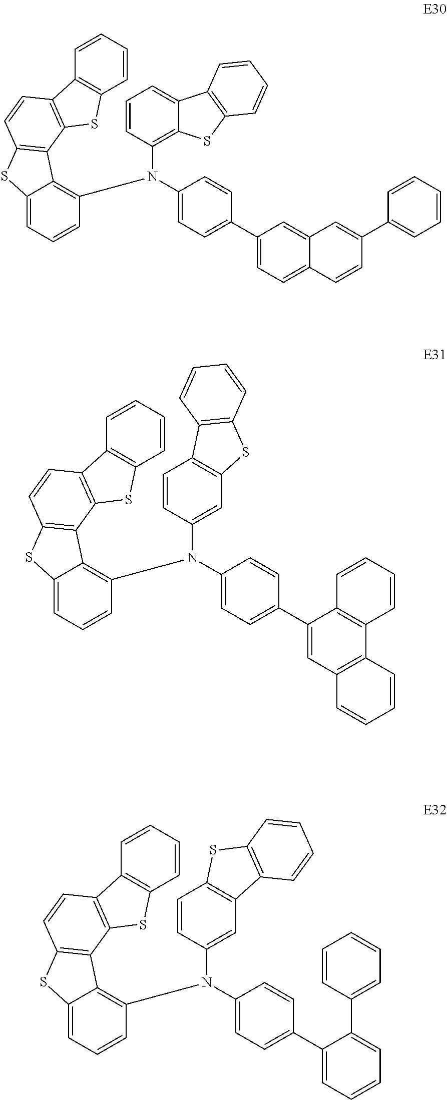 [SMILES: c1ccc(-c2ccc3ccc(-c4ccc(N(c5cccc6c5sc5ccccc56)c5cccc6sc7ccc8c9ccccc9sc8c7c56)cc4)cc3c2)cc1 ; c1ccc2c(c1)cc(-c1ccc(N(c3ccc4c(c3)sc3ccccc34)c3cccc4sc5ccc6c7ccccc7sc6c5c34)cc1)c1ccccc12 ; c1ccc(-c2ccccc2-c2ccc(N(c3ccc4sc5ccccc5c4c3)c3cccc4sc5ccc6c7ccccc7sc6c5c34)cc2)cc1]

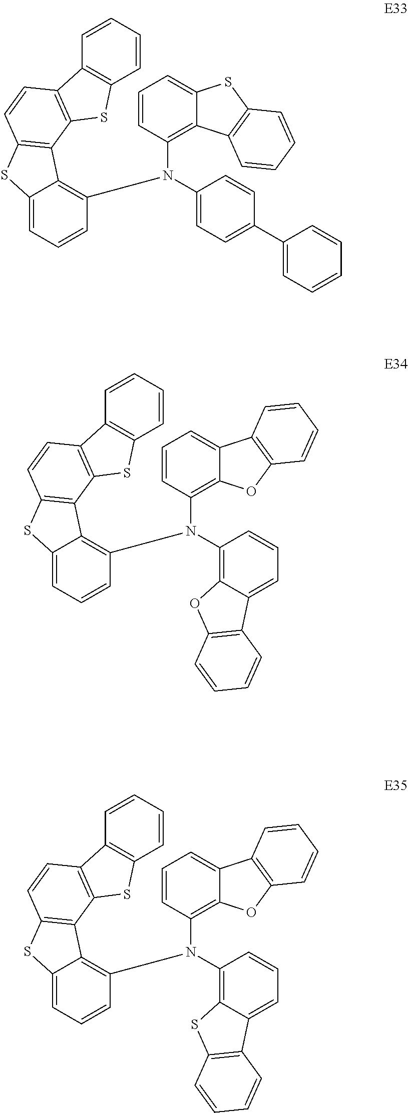 [SMILES: c1ccc(-c2ccc(N(c3cccc4sc5ccccc5c34)c3cccc4sc5ccc6c7ccccc7sc6c5c34)cc2)cc1 ; c1ccc2c(c1)oc1c(N(c3cccc4c3oc3ccccc34)c3cccc4sc5ccc6c7ccccc7sc6c5c34)cccc12 ; c1ccc2c(c1)oc1c(N(c3cccc4c3sc3ccccc34)c3cccc4sc5ccc6c7ccccc7sc6c5c34)cccc12]

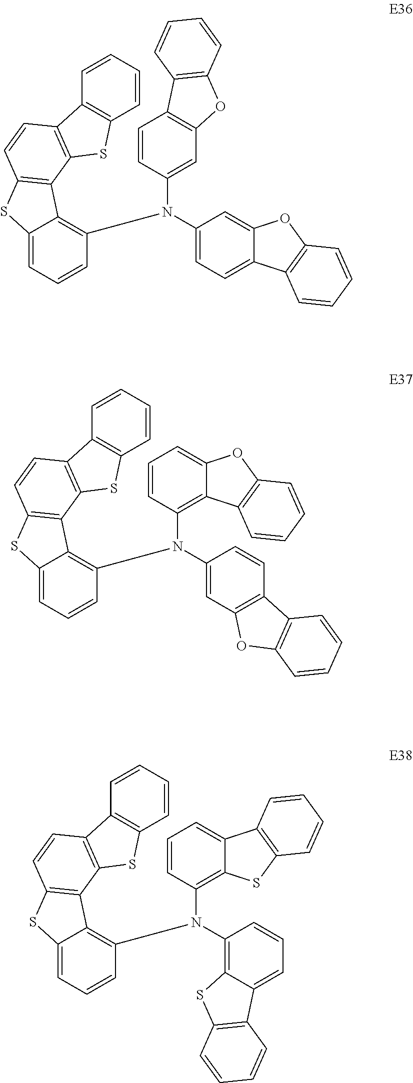 [SMILES: c1ccc2c(c1)oc1cc(N(c3ccc4c(c3)oc3ccccc34)c3cccc4sc5ccc6c7ccccc7sc6c5c34)ccc12 ; c1ccc2c(c1)oc1cc(N(c3cccc4oc5ccccc5c34)c3cccc4sc5ccc6c7ccccc7sc6c5c34)ccc12 ; c1ccc2c(c1)sc1c(N(c3cccc4c3sc3ccccc34)c3cccc4sc5ccc6c7ccccc7sc6c5c34)cccc12]

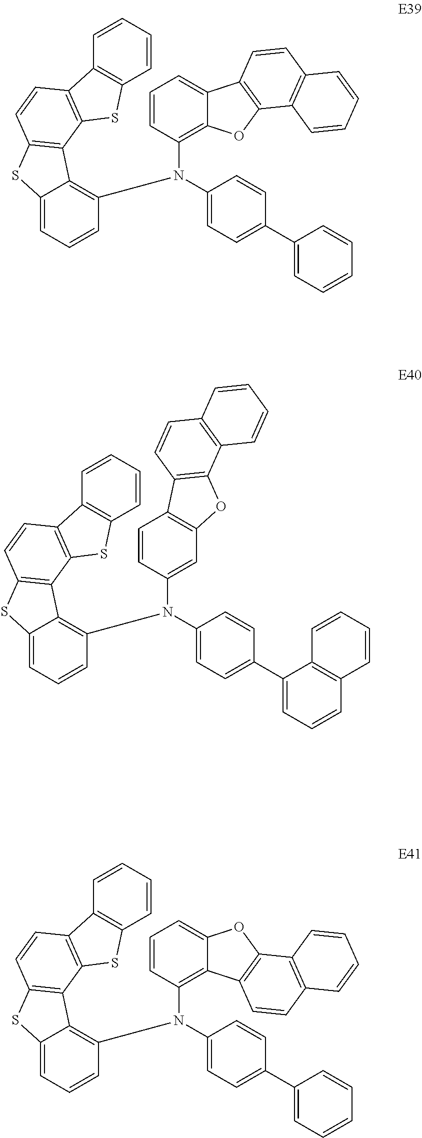 [SMILES: c1ccc(-c2ccc(N(c3cccc4c3oc3c5ccccc5ccc43)c3cccc4sc5ccc6c7ccccc7sc6c5c34)cc2)cc1 ; c1ccc2c(-c3ccc(N(c4ccc5c(c4)oc4c6ccccc6ccc54)c4cccc5sc6ccc7c8ccccc8sc7c6c45)cc3)cccc2c1 ; c1ccc(-c2ccc(N(c3cccc4oc5c6ccccc6ccc5c34)c3cccc4sc5ccc6c7ccccc7sc6c5c34)cc2)cc1]

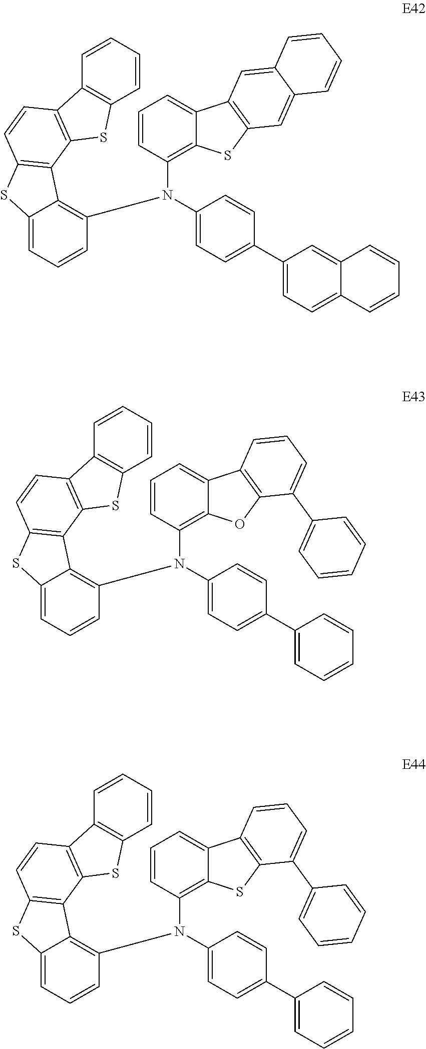 [SMILES: c1ccc2cc(-c3ccc(N(c4cccc5c4sc4cc6ccccc6cc45)c4cccc5sc6ccc7c8ccccc8sc7c6c45)cc3)ccc2c1 ; c1ccc(-c2ccc(N(c3cccc4c3oc3c(-c5ccccc5)cccc34)c3cccc4sc5ccc6c7ccccc7sc6c5c34)cc2)cc1 ; c1ccc(-c2ccc(N(c3cccc4c3sc3c(-c5ccccc5)cccc34)c3cccc4sc5ccc6c7ccccc7sc6c5c34)cc2)cc1]

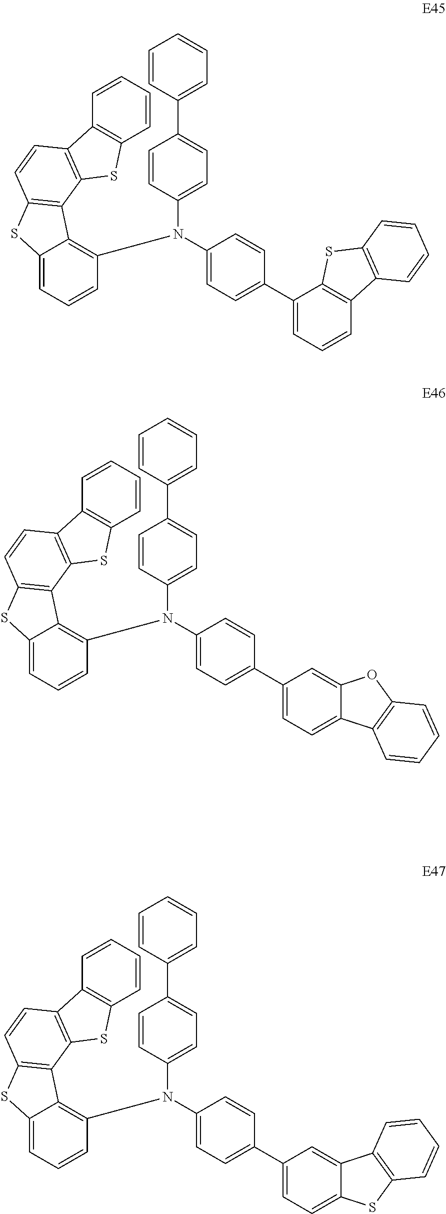 [SMILES: c1ccc(-c2ccc(N(c3ccc(-c4cccc5c4sc4ccccc45)cc3)c3cccc4sc5ccc6c7ccccc7sc6c5c34)cc2)cc1 ; c1ccc(-c2ccc(N(c3ccc(-c4ccc5c(c4)oc4ccccc45)cc3)c3cccc4sc5ccc6c7ccccc7sc6c5c34)cc2)cc1 ; c1ccc(-c2ccc(N(c3ccc(-c4ccc5sc6ccccc6c5c4)cc3)c3cccc4sc5ccc6c7ccccc7sc6c5c34)cc2)cc1]

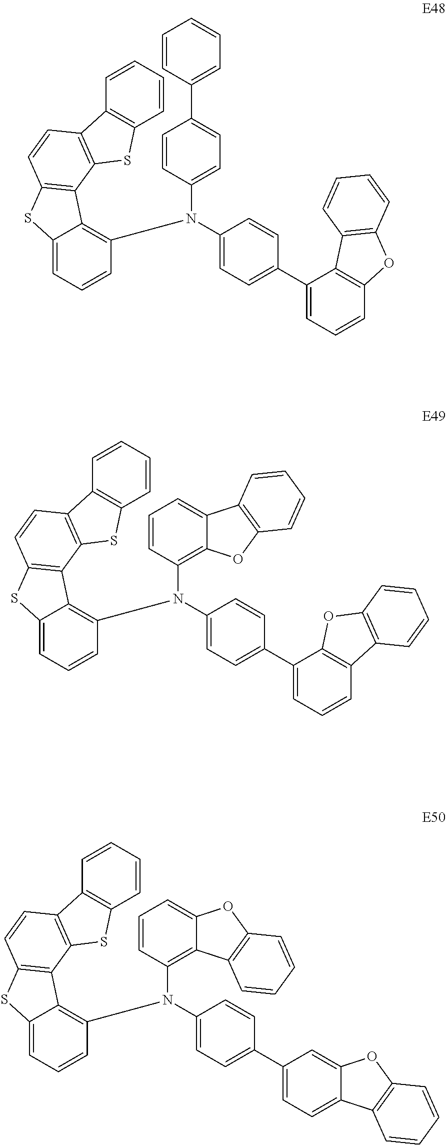 [SMILES: c1ccc(-c2ccc(N(c3ccc(-c4cccc5oc6ccccc6c45)cc3)c3cccc4sc5ccc6c7ccccc7sc6c5c34)cc2)cc1 ; c1ccc2c(c1)oc1c(-c3ccc(N(c4cccc5c4oc4ccccc45)c4cccc5sc6ccc7c8ccccc8sc7c6c45)cc3)cccc12 ; c1ccc2c(c1)oc1cc(-c3ccc(N(c4cccc5oc6ccccc6c45)c4cccc5sc6ccc7c8ccccc8sc7c6c45)cc3)ccc12]

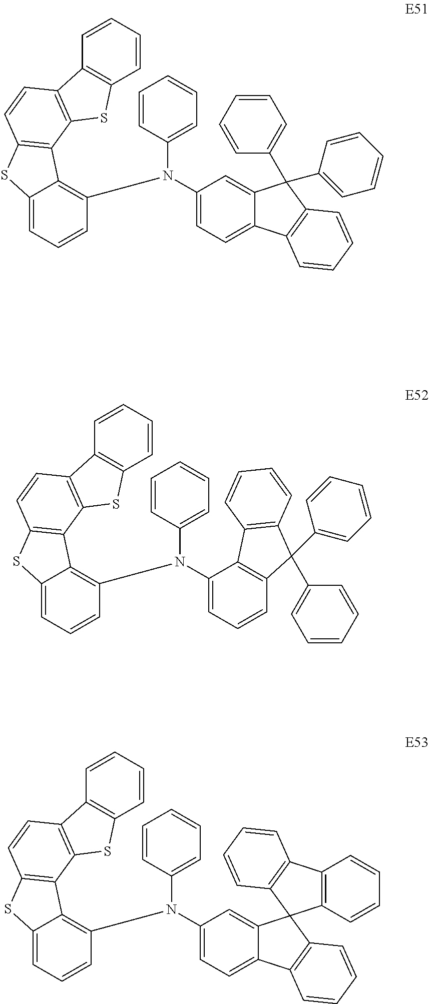 [SMILES: c1ccc(N(c2ccc3c(c2)C(c2ccccc2)(c2ccccc2)c2ccccc2-3)c2cccc3sc4ccc5c6ccccc6sc5c4c23)cc1 ; c1ccc(N(c2cccc3c2-c2ccccc2C3(c2ccccc2)c2ccccc2)c2cccc3sc4ccc5c6ccccc6sc5c4c23)cc1 ; c1ccc(N(c2ccc3c(c2)C2(c4ccccc4-c4ccccc42)c2ccccc2-3)c2cccc3sc4ccc5c6ccccc6sc5c4c23)cc1]

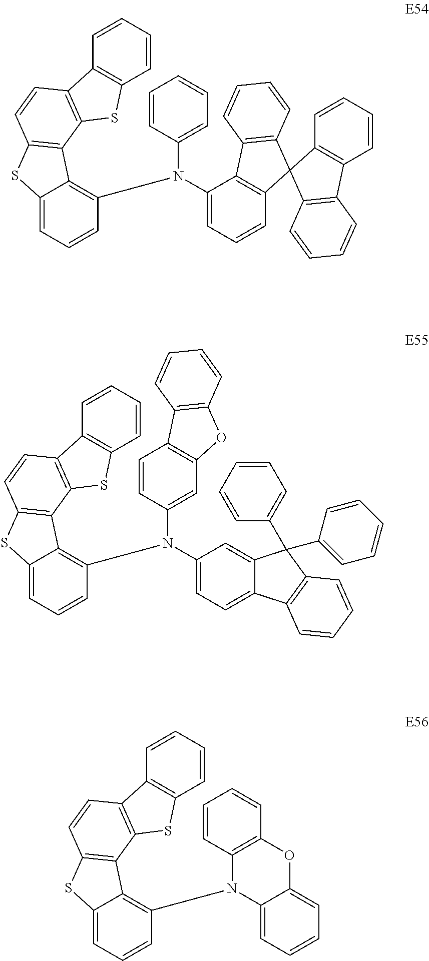 [SMILES: c1ccc(N(c2cccc3c2-c2ccccc2C32c3ccccc3-c3ccccc32)c2cccc3sc4ccc5c6ccccc6sc5c4c23)cc1 ; c1ccc(C2(c3ccccc3)c3ccccc3-c3ccc(N(c4ccc5c(c4)oc4ccccc45)c4cccc5sc6ccc7c8ccccc8sc7c6c45)cc32)cc1 ; c1ccc2c(c1)Oc1ccccc1N2c1cccc2sc3ccc4c5ccccc5sc4c3c12]

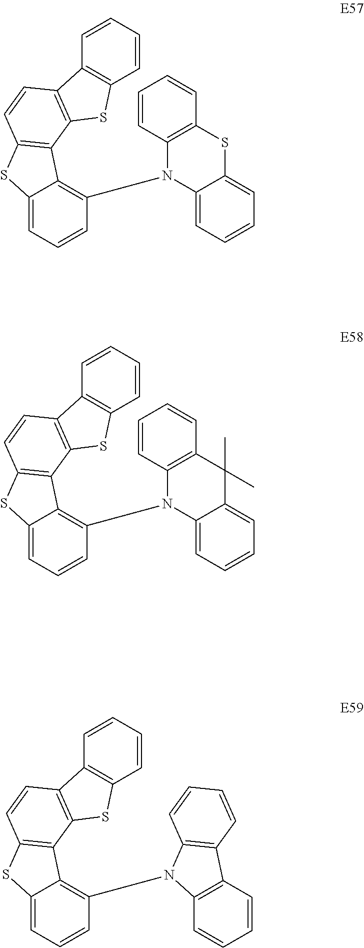 [SMILES: c1ccc2c(c1)Sc1ccccc1N2c1cccc2sc3ccc4c5ccccc5sc4c3c12 ; CC1(C)c2ccccc2N(c2cccc3sc4ccc5c6ccccc6sc5c4c23)c2ccccc21 ; c1ccc2c(c1)sc1c2ccc2sc3cccc(-n4c5ccccc5c5ccccc54)c3c21]

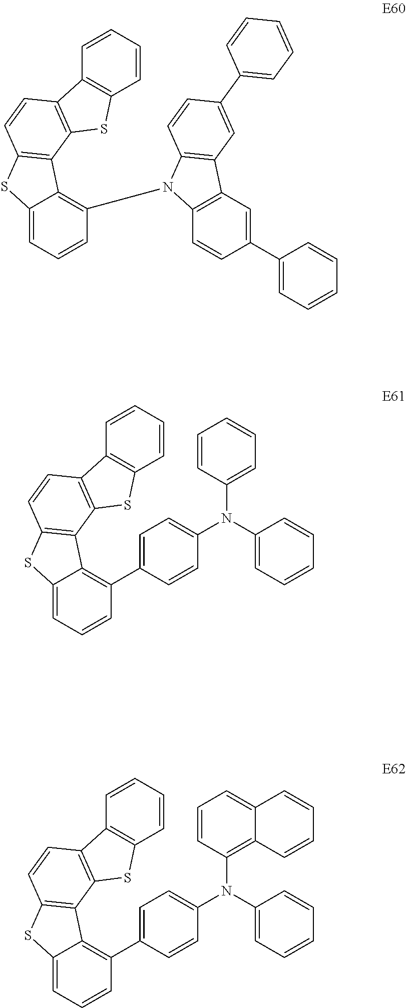 [SMILES: c1ccc(-c2ccc3c(c2)c2cc(-c4ccccc4)ccc2n3-c2cccc3sc4ccc5c6ccccc6sc5c4c23)cc1 ; c1ccc(N(c2ccccc2)c2ccc(-c3cccc4sc5ccc6c7ccccc7sc6c5c34)cc2)cc1 ; c1ccc(N(c2ccc(-c3cccc4sc5ccc6c7ccccc7sc6c5c34)cc2)c2cccc3ccccc23)cc1]

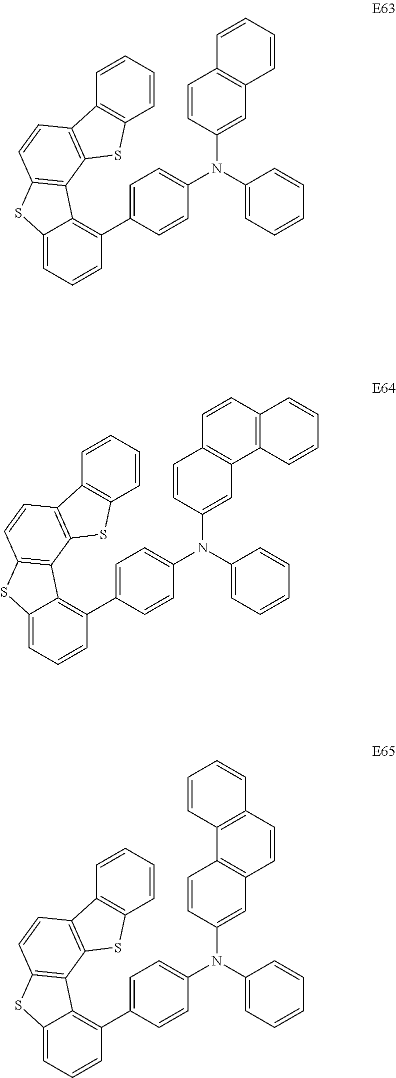 [SMILES: c1ccc(N(c2ccc(-c3cccc4sc5ccc6c7ccccc7sc6c5c34)cc2)c2ccc3ccccc3c2)cc1 ; c1ccc(N(c2ccc(-c3cccc4sc5ccc6c7ccccc7sc6c5c34)cc2)c2ccc3ccc4ccccc4c3c2)cc1 ; c1ccc(N(c2ccc(-c3cccc4sc5ccc6c7ccccc7sc6c5c34)cc2)c2ccc3c(ccc4ccccc43)c2)cc1]

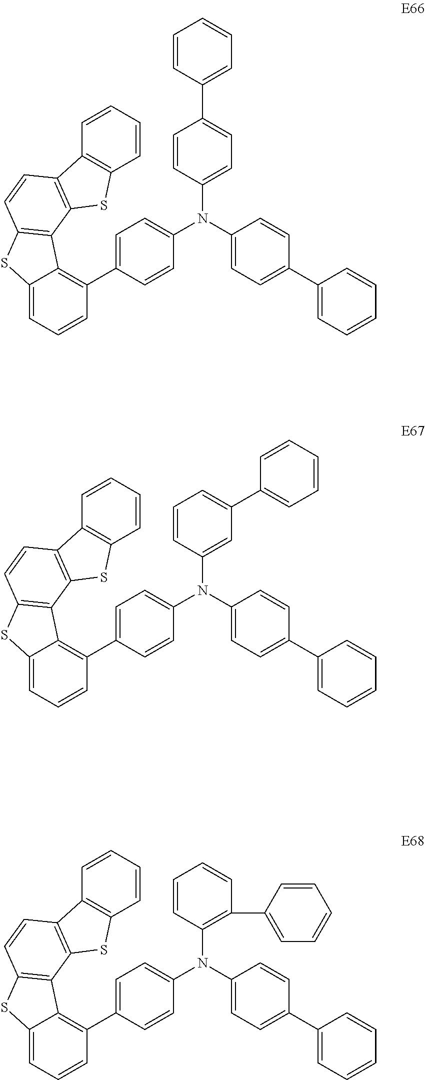 [SMILES: c1ccc(-c2ccc(N(c3ccc(-c4ccccc4)cc3)c3ccc(-c4cccc5sc6ccc7c8ccccc8sc7c6c45)cc3)cc2)cc1 ; c1ccc(-c2ccc(N(c3ccc(-c4cccc5sc6ccc7c8ccccc8sc7c6c45)cc3)c3cccc(-c4ccccc4)c3)cc2)cc1 ; c1ccc(-c2ccc(N(c3ccc(-c4cccc5sc6ccc7c8ccccc8sc7c6c45)cc3)c3ccccc3-c3ccccc3)cc2)cc1]

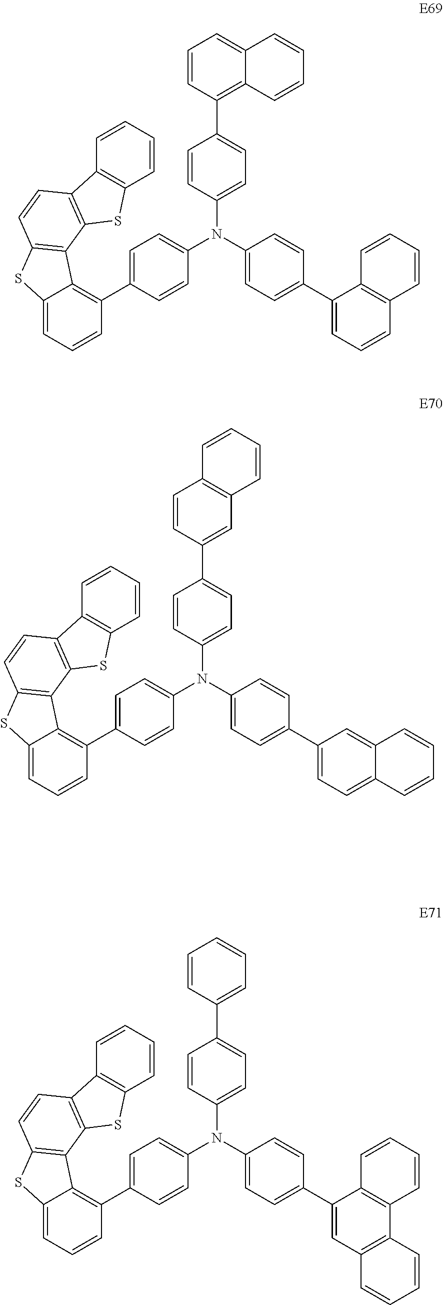 [SMILES: c1ccc2c(-c3ccc(N(c4ccc(-c5cccc6ccccc56)cc4)c4ccc(-c5cccc6sc7ccc8c9ccccc9sc8c7c56)cc4)cc3)cccc2c1 ; c1ccc2cc(-c3ccc(N(c4ccc(-c5ccc6ccccc6c5)cc4)c4ccc(-c5cccc6sc7ccc8c9ccccc9sc8c7c56)cc4)cc3)ccc2c1 ; c1ccc(-c2ccc(N(c3ccc(-c4cc5ccccc5c5ccccc45)cc3)c3ccc(-c4cccc5sc6ccc7c8ccccc8sc7c6c45)cc3)cc2)cc1]

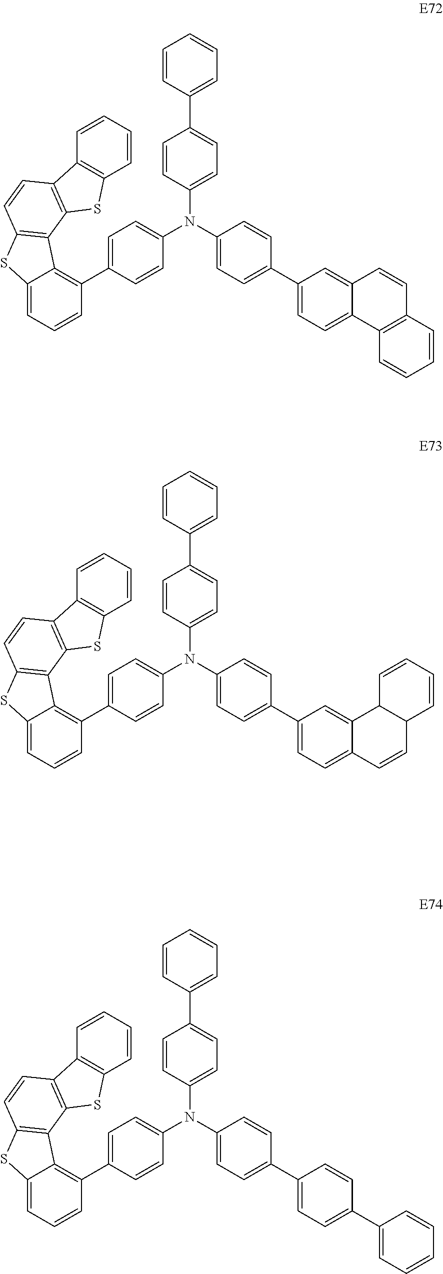 [SMILES: c1ccc(-c2ccc(N(c3ccc(-c4ccc5c(ccc6ccccc65)c4)cc3)c3ccc(-c4cccc5sc6ccc7c8ccccc8sc7c6c45)cc3)cc2)cc1 ; C1=CC2C=Cc3ccc(-c4ccc(N(c5ccc(-c6ccccc6)cc5)c5ccc(-c6cccc7sc8ccc9c%10ccccc%10sc9c8c67)cc5)cc4)cc3C2C=C1 ; c1ccc(-c2ccc(-c3ccc(N(c4ccc(-c5ccccc5)cc4)c4ccc(-c5cccc6sc7ccc8c9ccccc9sc8c7c56)cc4)cc3)cc2)cc1]

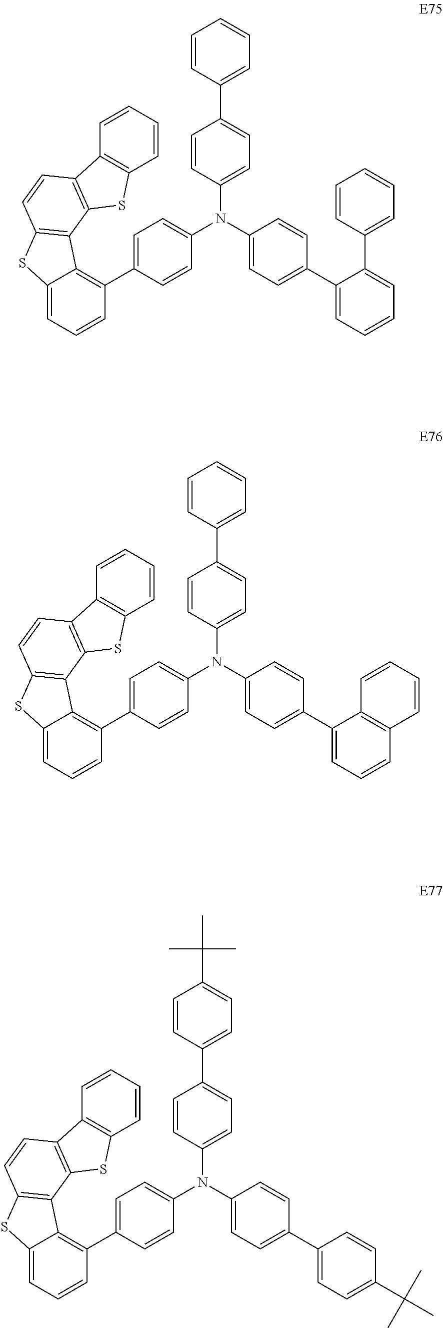 [SMILES: c1ccc(-c2ccc(N(c3ccc(-c4ccccc4-c4ccccc4)cc3)c3ccc(-c4cccc5sc6ccc7c8ccccc8sc7c6c45)cc3)cc2)cc1 ; c1ccc(-c2ccc(N(c3ccc(-c4cccc5ccccc45)cc3)c3ccc(-c4cccc5sc6ccc7c8ccccc8sc7c6c45)cc3)cc2)cc1 ; CC(C)(C)c1ccc(-c2ccc(N(c3ccc(-c4ccc(C(C)(C)C)cc4)cc3)c3ccc(-c4cccc5sc6ccc7c8ccccc8sc7c6c45)cc3)cc2)cc1]

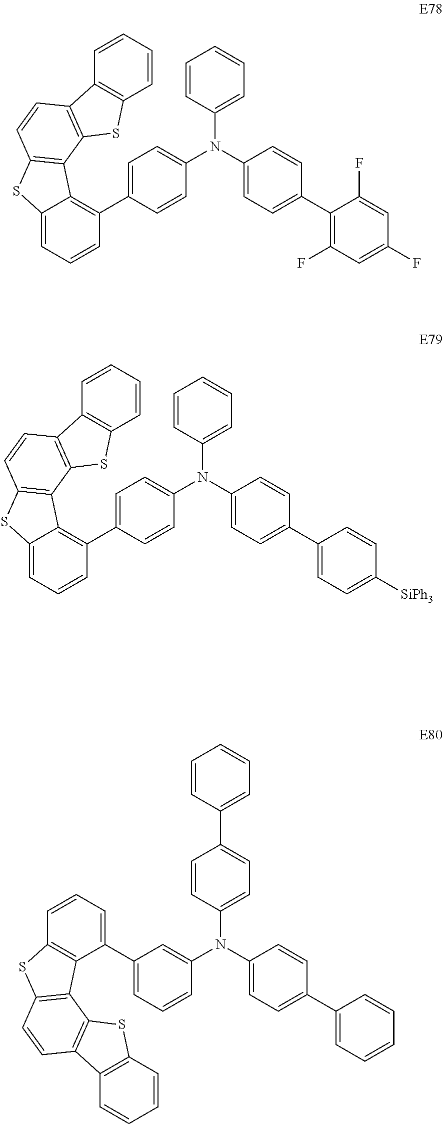

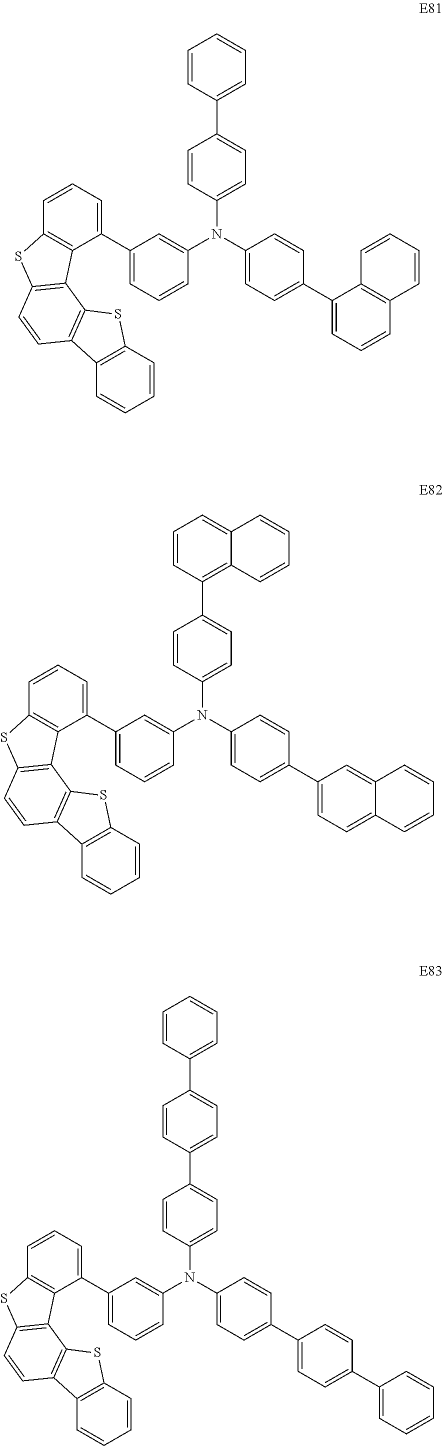 [SMILES: c1ccc(-c2ccc(N(c3ccc(-c4cccc5ccccc45)cc3)c3cccc(-c4cccc5sc6ccc7c8ccccc8sc7c6c45)c3)cc2)cc1 ; c1cc(-c2cccc3sc4ccc5c6ccccc6sc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6sc7ccc8c9ccccc9sc8c7c56)c4)cc3)cc2)cc1]

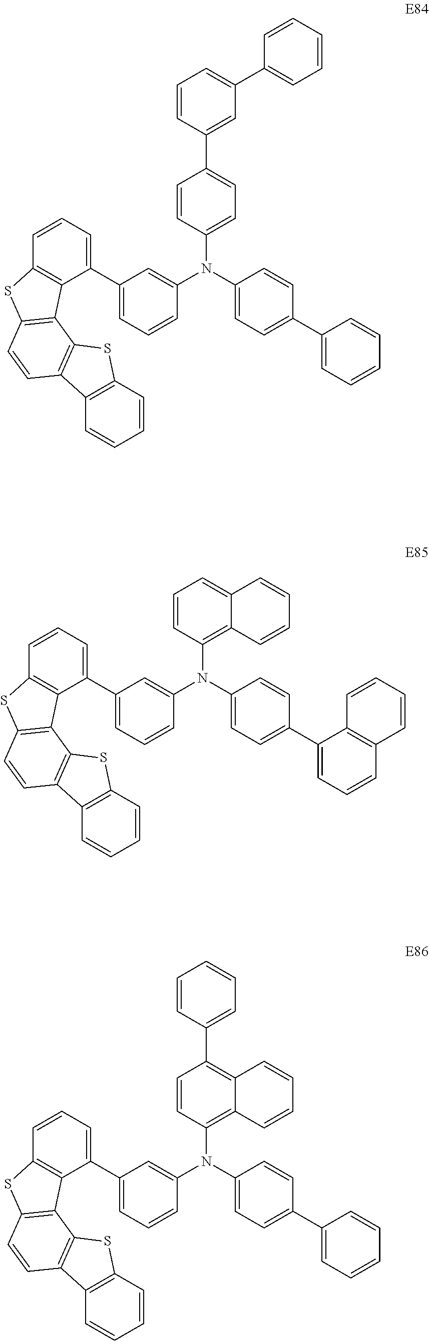 [SMILES: c1ccc(-c2ccc(N(c3ccc(-c4cccc(-c5ccccc5)c4)cc3)c3cccc(-c4cccc5sc6ccc7c8ccccc8sc7c6c45)c3)cc2)cc1 ; c1cc(-c2cccc3sc4ccc5c6ccccc6sc5c4c23)cc(N(c2ccc(-c3cccc4ccccc34)cc2)c2cccc3ccccc23)c1 ; c1ccc(-c2ccc(N(c3cccc(-c4cccc5sc6ccc7c8ccccc8sc7c6c45)c3)c3ccc(-c4ccccc4)c4ccccc34)cc2)cc1]

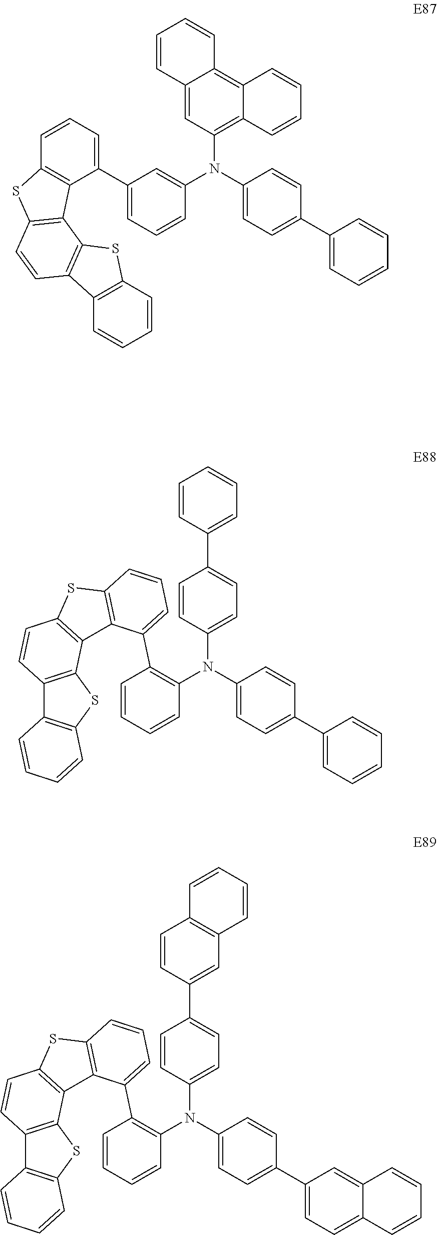 [SMILES: c1ccc(-c2ccc(N(c3cccc(-c4cccc5sc6ccc7c8ccccc8sc7c6c45)c3)c3cc4ccccc4c4ccccc34)cc2)cc1 ; c1ccc(-c2ccc(N(c3ccc(-c4ccccc4)cc3)c3ccccc3-c3cccc4sc5ccc6c7ccccc7sc6c5c34)cc2)cc1 ; c1ccc(N(c2ccc(-c3ccc4ccccc4c3)cc2)c2ccc(-c3ccc4ccccc4c3)cc2)c(-c2cccc3sc4ccc5c6ccccc6sc5c4c23)c1]

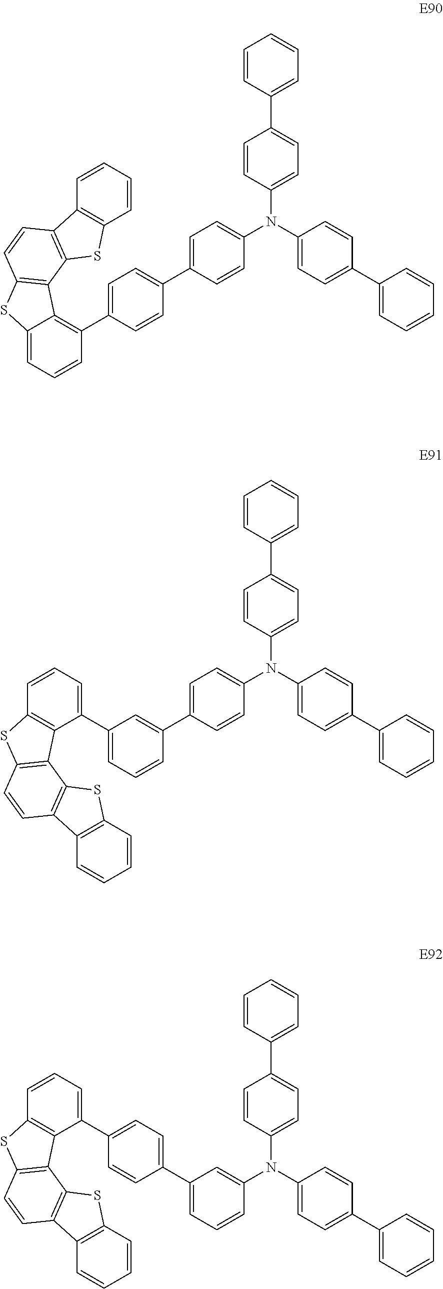 [SMILES: c1ccc(-c2ccc(N(c3ccc(-c4ccccc4)cc3)c3ccc(-c4ccc(-c5cccc6sc7ccc8c9ccccc9sc8c7c56)cc4)cc3)cc2)cc1 ; c1ccc(-c2ccc(N(c3ccc(-c4ccccc4)cc3)c3ccc(-c4cccc(-c5cccc6sc7ccc8c9ccccc9sc8c7c56)c4)cc3)cc2)cc1 ; c1ccc(-c2ccc(N(c3ccc(-c4ccccc4)cc3)c3cccc(-c4ccc(-c5cccc6sc7ccc8c9ccccc9sc8c7c56)cc4)c3)cc2)cc1]

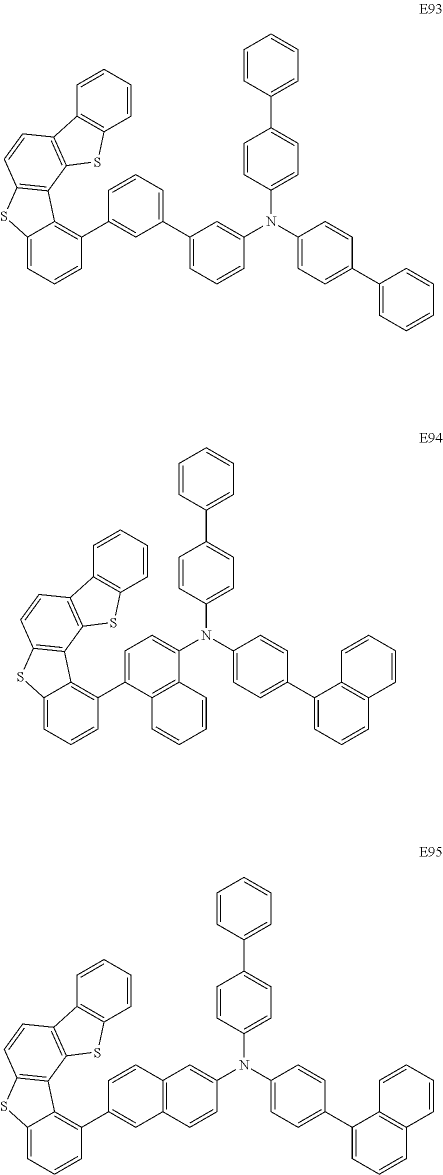 [SMILES: c1ccc(-c2ccc(N(c3ccc(-c4ccccc4)cc3)c3cccc(-c4cccc(-c5cccc6sc7ccc8c9ccccc9sc8c7c56)c4)c3)cc2)cc1 ; c1ccc(-c2ccc(N(c3ccc(-c4cccc5ccccc45)cc3)c3ccc(-c4cccc5sc6ccc7c8ccccc8sc7c6c45)c4ccccc34)cc2)cc1 ; c1ccc(-c2ccc(N(c3ccc(-c4cccc5ccccc45)cc3)c3ccc4cc(-c5cccc6sc7ccc8c9ccccc9sc8c7c56)ccc4c3)cc2)cc1]

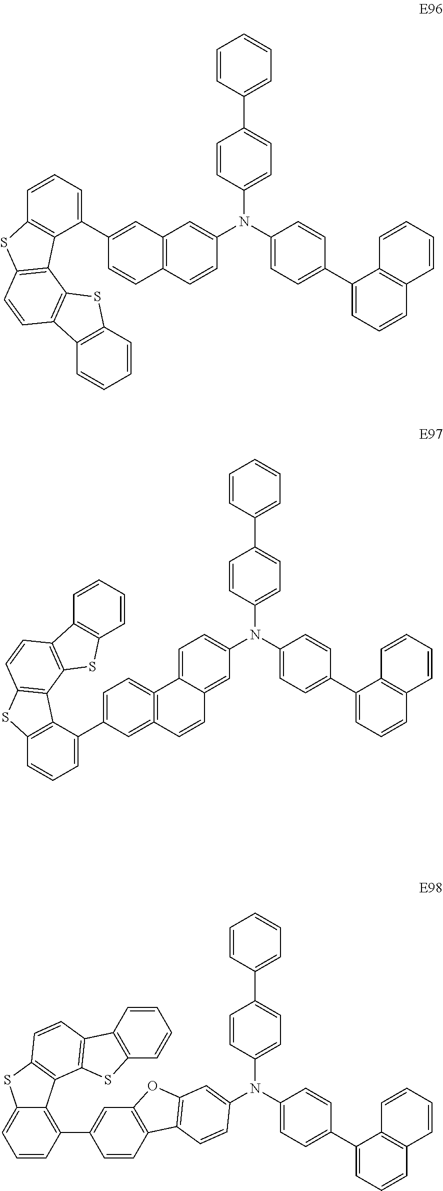

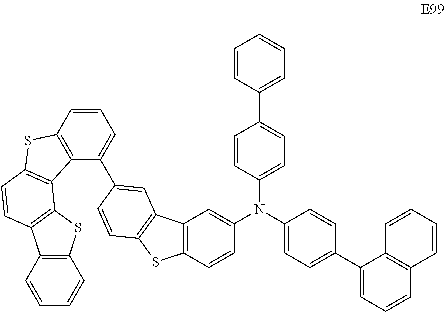 [SMILES: c1ccc(-c2ccc(N(c3ccc(-c4cccc5ccccc45)cc3)c3ccc4sc5ccc(-c6cccc7sc8ccc9c%10ccccc%10sc9c8c67)cc5c4c3)cc2)cc1]

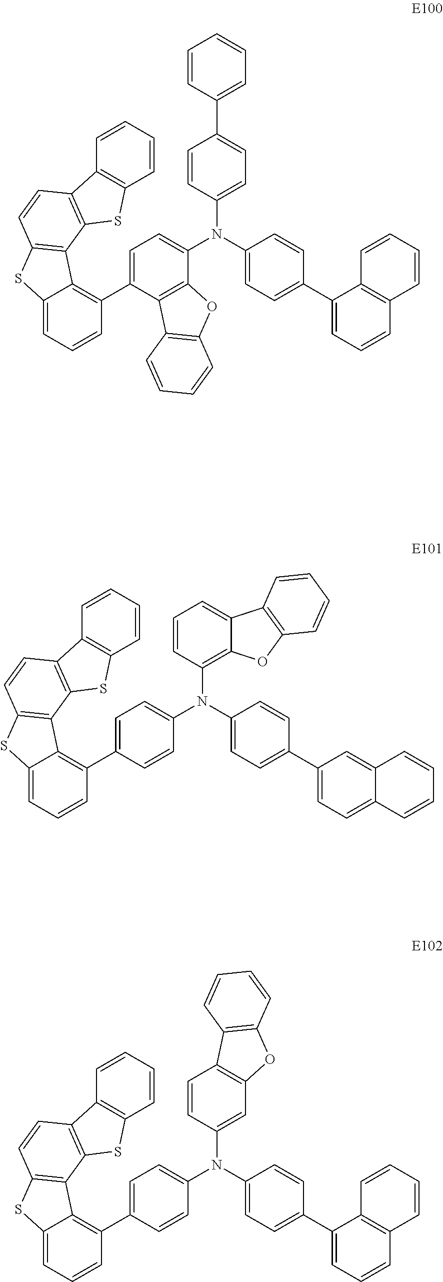 [SMILES: c1ccc(-c2ccc(N(c3ccc(-c4cccc5ccccc45)cc3)c3ccc(-c4cccc5sc6ccc7c8ccccc8sc7c6c45)c4c3oc3ccccc34)cc2)cc1 ; c1ccc2cc(-c3ccc(N(c4ccc(-c5cccc6sc7ccc8c9ccccc9sc8c7c56)cc4)c4cccc5c4oc4ccccc45)cc3)ccc2c1 ; c1ccc2c(-c3ccc(N(c4ccc(-c5cccc6sc7ccc8c9ccccc9sc8c7c56)cc4)c4ccc5c(c4)oc4ccccc45)cc3)cccc2c1]

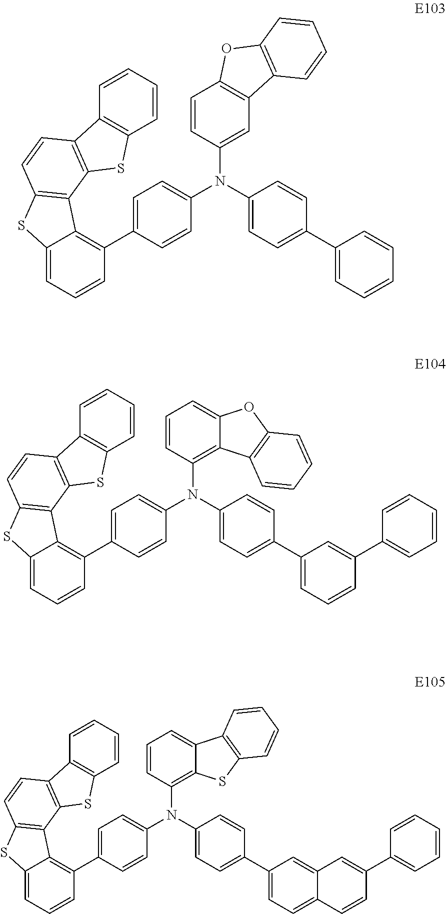 [SMILES: c1ccc(-c2ccc(N(c3ccc(-c4cccc5sc6ccc7c8ccccc8sc7c6c45)cc3)c3ccc4oc5ccccc5c4c3)cc2)cc1 ; c1ccc(-c2cccc(-c3ccc(N(c4ccc(-c5cccc6sc7ccc8c9ccccc9sc8c7c56)cc4)c4cccc5oc6ccccc6c45)cc3)c2)cc1 ; c1ccc(-c2ccc3ccc(-c4ccc(N(c5ccc(-c6cccc7sc8ccc9c%10ccccc%10sc9c8c67)cc5)c5cccc6c5sc5ccccc56)cc4)cc3c2)cc1]

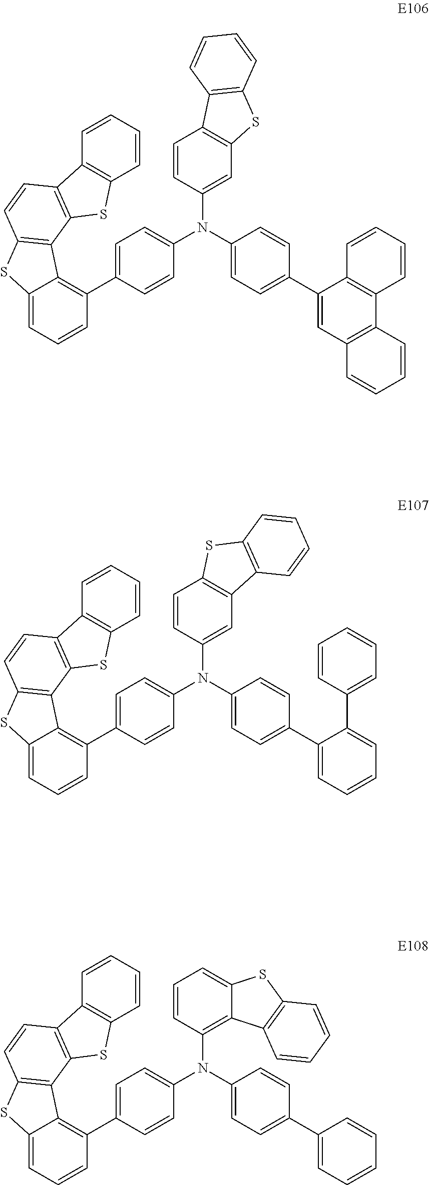 [SMILES: c1ccc2c(c1)cc(-c1ccc(N(c3ccc(-c4cccc5sc6ccc7c8ccccc8sc7c6c45)cc3)c3ccc4c(c3)sc3ccccc34)cc1)c1ccccc12 ; c1ccc(-c2ccccc2-c2ccc(N(c3ccc(-c4cccc5sc6ccc7c8ccccc8sc7c6c45)cc3)c3ccc4sc5ccccc5c4c3)cc2)cc1 ; c1ccc(-c2ccc(N(c3ccc(-c4cccc5sc6ccc7c8ccccc8sc7c6c45)cc3)c3cccc4sc5ccccc5c34)cc2)cc1]

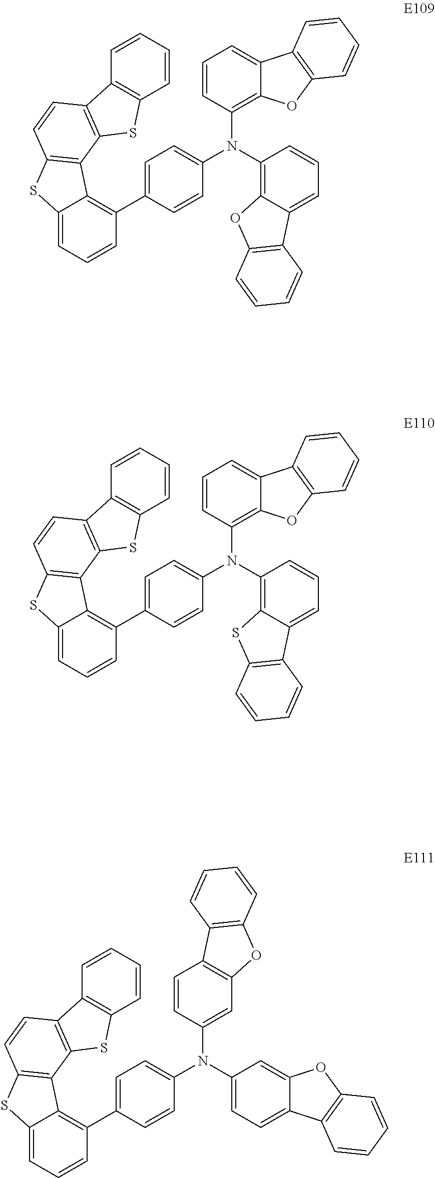 [SMILES: c1ccc2c(c1)oc1c(N(c3ccc(-c4cccc5sc6ccc7c8ccccc8sc7c6c45)cc3)c3cccc4c3oc3ccccc34)cccc12 ; c1ccc2c(c1)oc1c(N(c3ccc(-c4cccc5sc6ccc7c8ccccc8sc7c6c45)cc3)c3cccc4c3sc3ccccc34)cccc12 ; c1ccc2c(c1)oc1cc(N(c3ccc(-c4cccc5sc6ccc7c8ccccc8sc7c6c45)cc3)c3ccc4c(c3)oc3ccccc34)ccc12]

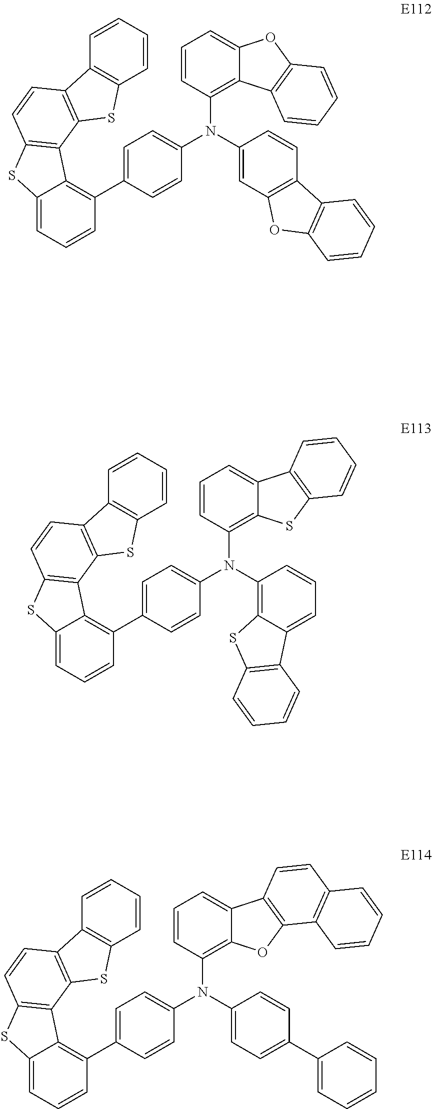 [SMILES: c1ccc2c(c1)oc1cc(N(c3ccc(-c4cccc5sc6ccc7c8ccccc8sc7c6c45)cc3)c3cccc4oc5ccccc5c34)ccc12 ; c1ccc2c(c1)sc1c(N(c3ccc(-c4cccc5sc6ccc7c8ccccc8sc7c6c45)cc3)c3cccc4c3sc3ccccc34)cccc12 ; c1ccc(-c2ccc(N(c3ccc(-c4cccc5sc6ccc7c8ccccc8sc7c6c45)cc3)c3cccc4c3oc3c5ccccc5ccc43)cc2)cc1]

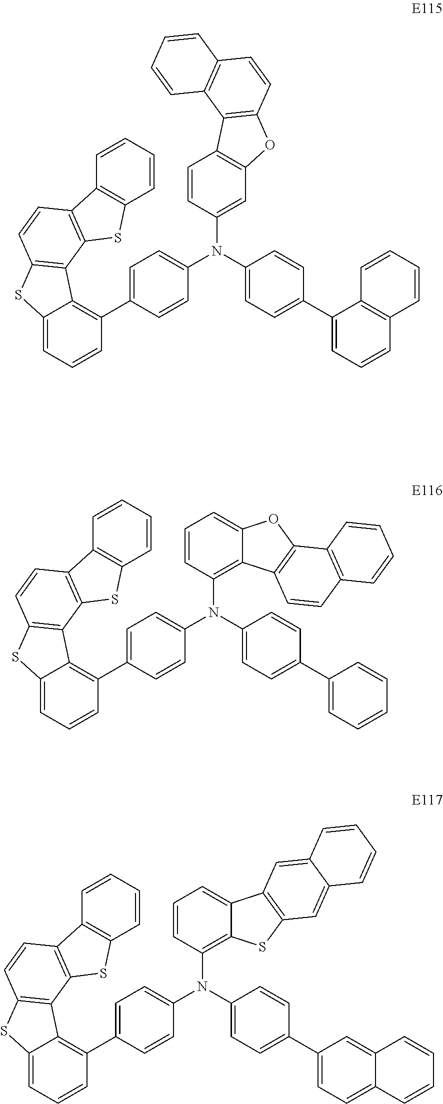 [SMILES: c1ccc2c(-c3ccc(N(c4ccc(-c5cccc6sc7ccc8c9ccccc9sc8c7c56)cc4)c4ccc5c(c4)oc4ccc6ccccc6c45)cc3)cccc2c1 ; c1ccc(-c2ccc(N(c3ccc(-c4cccc5sc6ccc7c8ccccc8sc7c6c45)cc3)c3cccc4oc5c6ccccc6ccc5c34)cc2)cc1 ; c1ccc2cc(-c3ccc(N(c4ccc(-c5cccc6sc7ccc8c9ccccc9sc8c7c56)cc4)c4cccc5c4sc4cc6ccccc6cc45)cc3)ccc2c1]

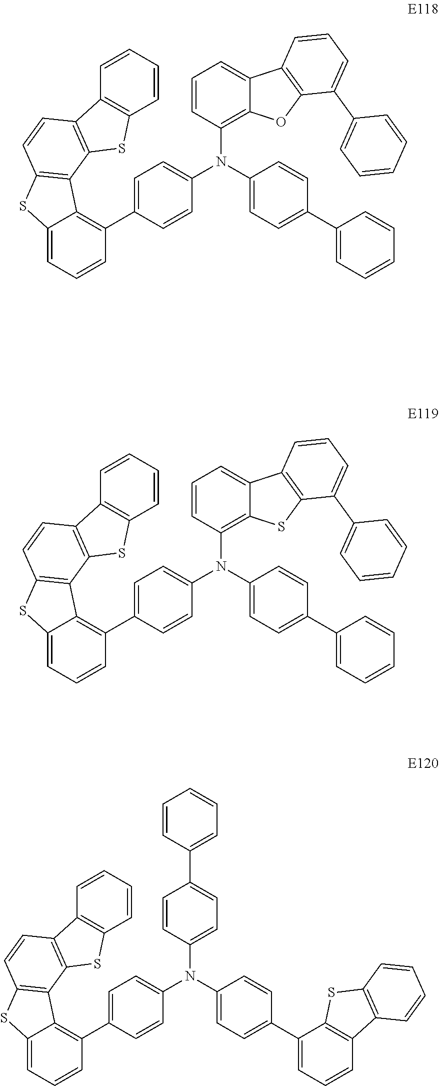 [SMILES: c1ccc(-c2ccc(N(c3ccc(-c4cccc5sc6ccc7c8ccccc8sc7c6c45)cc3)c3cccc4c3oc3c(-c5ccccc5)cccc34)cc2)cc1 ; c1ccc(-c2ccc(N(c3ccc(-c4cccc5sc6ccc7c8ccccc8sc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5sc6ccc7c8ccccc8sc7c6c45)cc3)cc2)cc1]

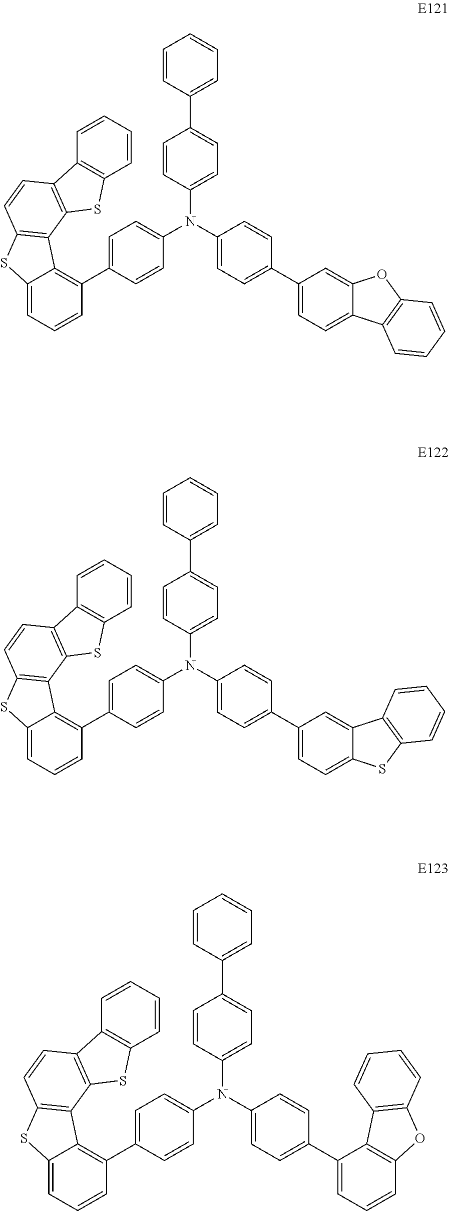 [SMILES: c1ccc(-c2ccc(N(c3ccc(-c4ccc5c(c4)oc4ccccc45)cc3)c3ccc(-c4cccc5sc6ccc7c8ccccc8sc7c6c45)cc3)cc2)cc1 ; c1ccc(-c2ccc(N(c3ccc(-c4ccc5sc6ccccc6c5c4)cc3)c3ccc(-c4cccc5sc6ccc7c8ccccc8sc7c6c45)cc3)cc2)cc1 ; c1ccc(-c2ccc(N(c3ccc(-c4cccc5oc6ccccc6c45)cc3)c3ccc(-c4cccc5sc6ccc7c8ccccc8sc7c6c45)cc3)cc2)cc1]

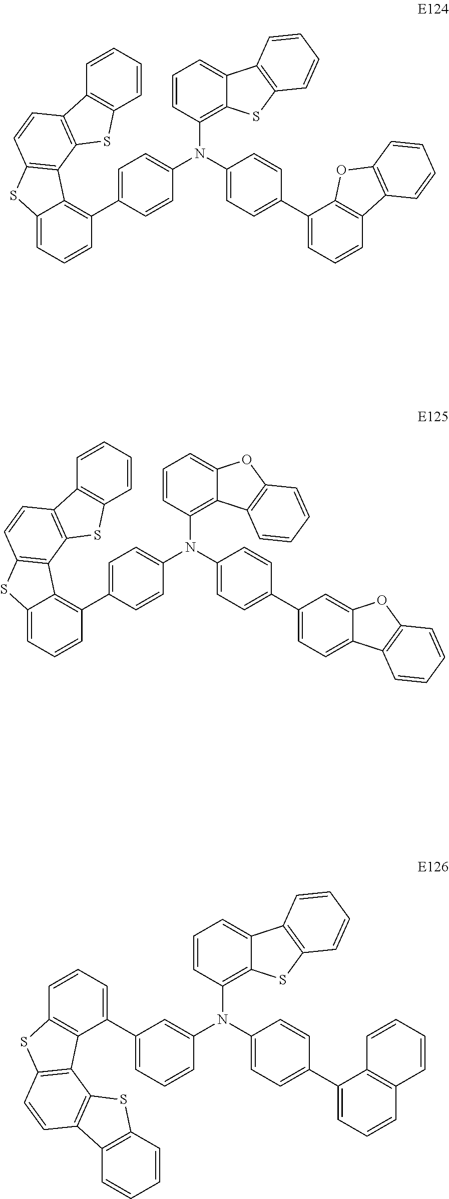 [SMILES: c1ccc2c(c1)oc1c(-c3ccc(N(c4ccc(-c5cccc6sc7ccc8c9ccccc9sc8c7c56)cc4)c4cccc5c4sc4ccccc45)cc3)cccc12 ; c1ccc2c(c1)oc1cc(-c3ccc(N(c4ccc(-c5cccc6sc7ccc8c9ccccc9sc8c7c56)cc4)c4cccc5oc6ccccc6c45)cc3)ccc12 ; c1cc(-c2cccc3sc4ccc5c6ccccc6sc5c4c23)cc(N(c2ccc(-c3cccc4ccccc34)cc2)c2cccc3c2sc2ccccc23)c1]

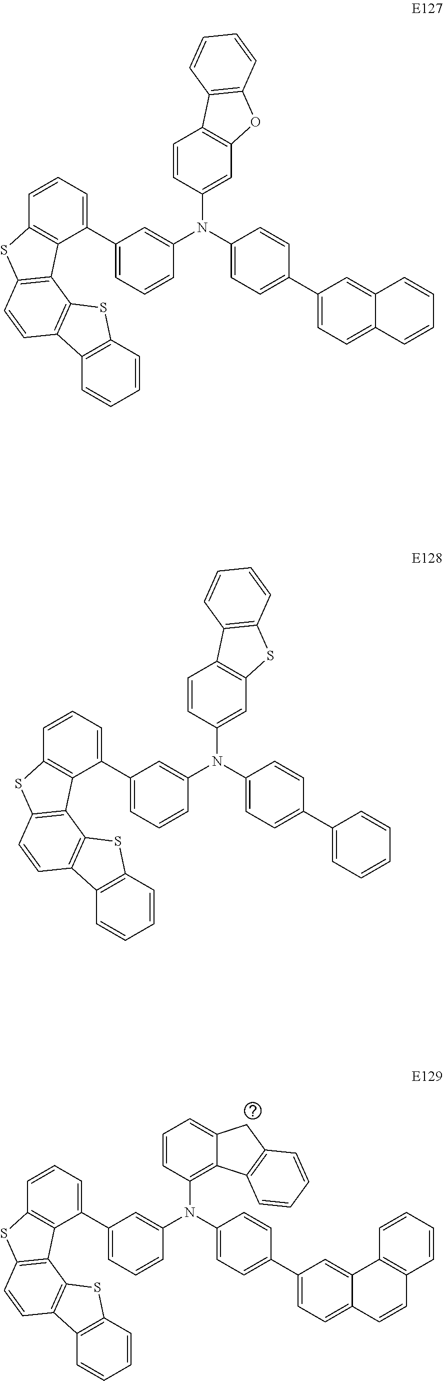 [SMILES: c1cc(-c2cccc3sc4ccc5c6ccccc6sc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5sc6ccc7c8ccccc8sc7c6c45)c3)c3ccc4c(c3)sc3ccccc34)cc2)cc1 ; c1cc(-c2cccc3sc4ccc5c6ccccc6sc5c4c23)cc(N(c2ccc(-c3ccc4ccc5ccccc5c4c3)cc2)c2cccc3c2-c2ccccc2C3)c1]

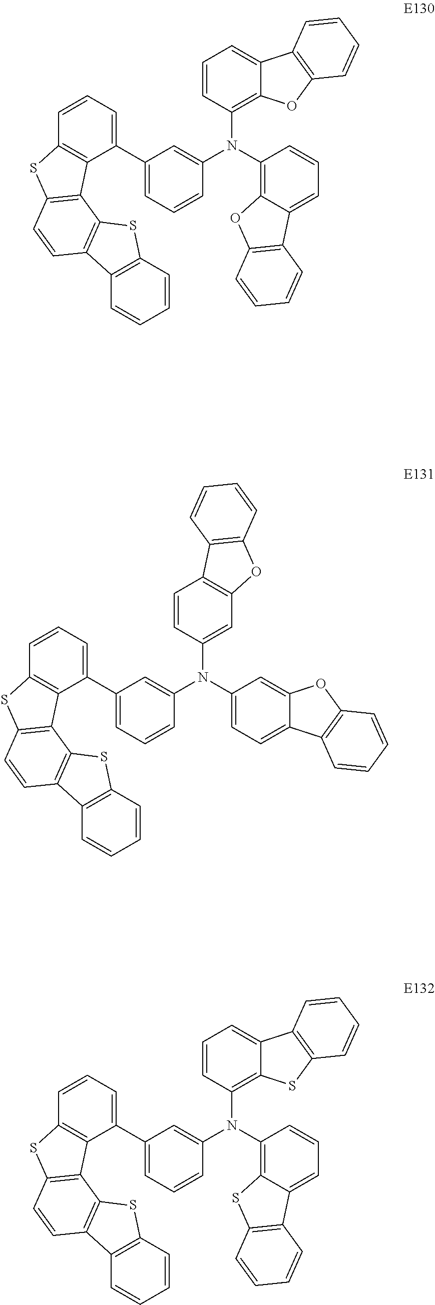 [SMILES: c1cc(-c2cccc3sc4ccc5c6ccccc6sc5c4c23)cc(N(c2cccc3c2oc2ccccc23)c2cccc3c2oc2ccccc23)c1 ; c1cc(-c2cccc3sc4ccc5c6ccccc6sc5c4c23)cc(N(c2ccc3c(c2)oc2ccccc23)c2ccc3c(c2)oc2ccccc23)c1 ; c1cc(-c2cccc3sc4ccc5c6ccccc6sc5c4c23)cc(N(c2cccc3c2sc2ccccc23)c2cccc3c2sc2ccccc23)c1]

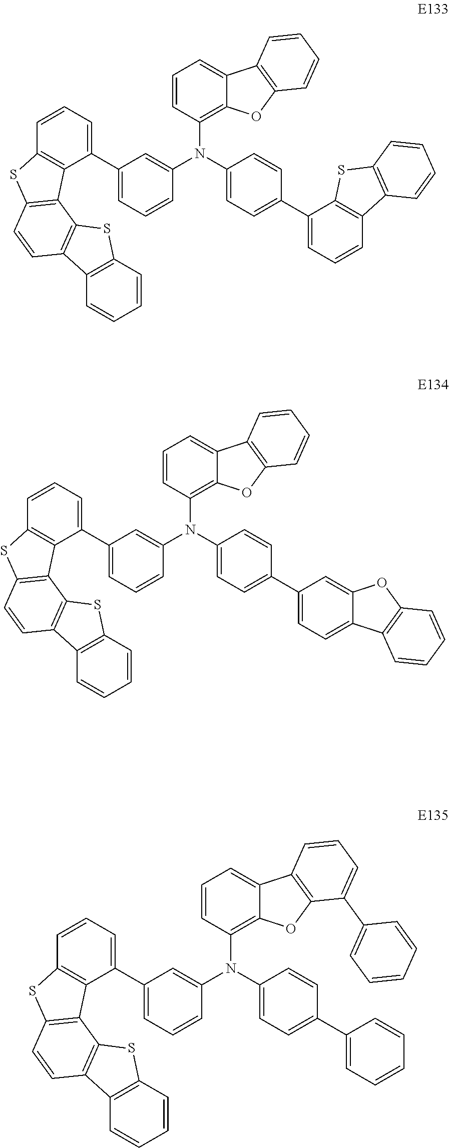 [SMILES: c1cc(-c2cccc3sc4ccc5c6ccccc6sc5c4c23)cc(N(c2ccc(-c3cccc4c3sc3ccccc34)cc2)c2cccc3c2oc2ccccc23)c1 ; c1cc(-c2cccc3sc4ccc5c6ccccc6sc5c4c23)cc(N(c2ccc(-c3ccc4c(c3)oc3ccccc34)cc2)c2cccc3c2oc2ccccc23)c1 ; c1ccc(-c2ccc(N(c3cccc(-c4cccc5sc6ccc7c8ccccc8sc7c6c45)c3)c3cccc4c3oc3c(-c5ccccc5)cccc34)cc2)cc1]

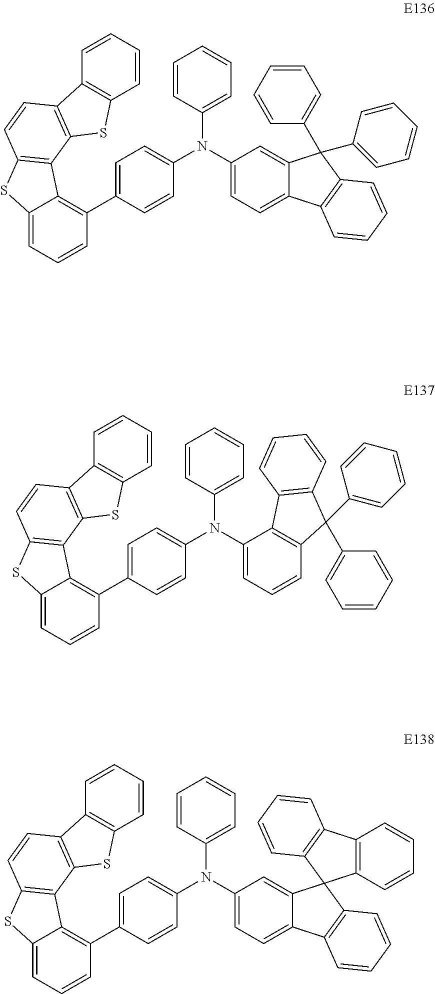 [SMILES: c1ccc(N(c2ccc(-c3cccc4sc5ccc6c7ccccc7sc6c5c34)cc2)c2ccc3c(c2)C(c2ccccc2)(c2ccccc2)c2ccccc2-3)cc1 ; c1ccc(N(c2ccc(-c3cccc4sc5ccc6c7ccccc7sc6c5c34)cc2)c2cccc3c2-c2ccccc2C3(c2ccccc2)c2ccccc2)cc1 ; c1ccc(N(c2ccc(-c3cccc4sc5ccc6c7ccccc7sc6c5c34)cc2)c2ccc3c(c2)C2(c4ccccc4-c4ccccc42)c2ccccc2-3)cc1]

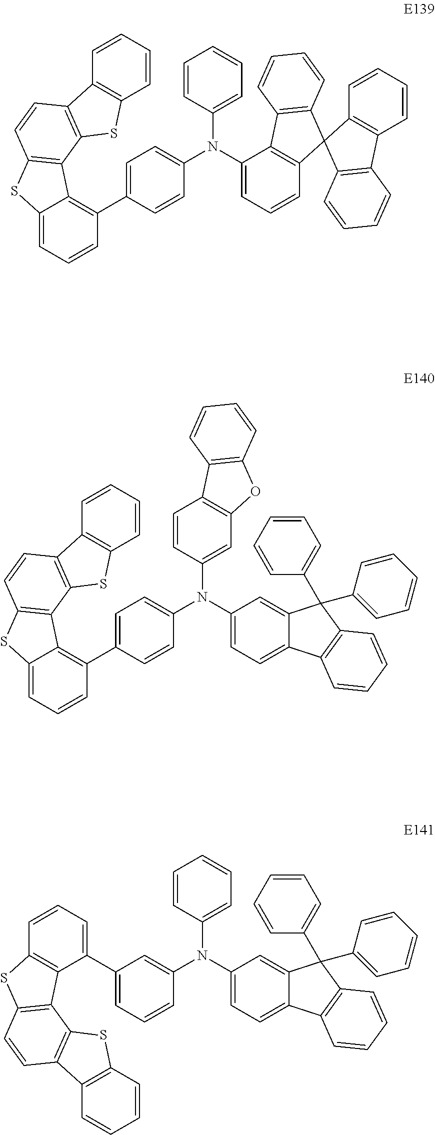 [SMILES: c1ccc(N(c2ccc(-c3cccc4sc5ccc6c7ccccc7sc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6sc7ccc8c9ccccc9sc8c7c56)cc4)c4ccc5c(c4)oc4ccccc45)cc32)cc1 ; c1ccc(N(c2cccc(-c3cccc4sc5ccc6c7ccccc7sc6c5c34)c2)c2ccc3c(c2)C(c2ccccc2)(c2ccccc2)c2ccccc2-3)cc1]

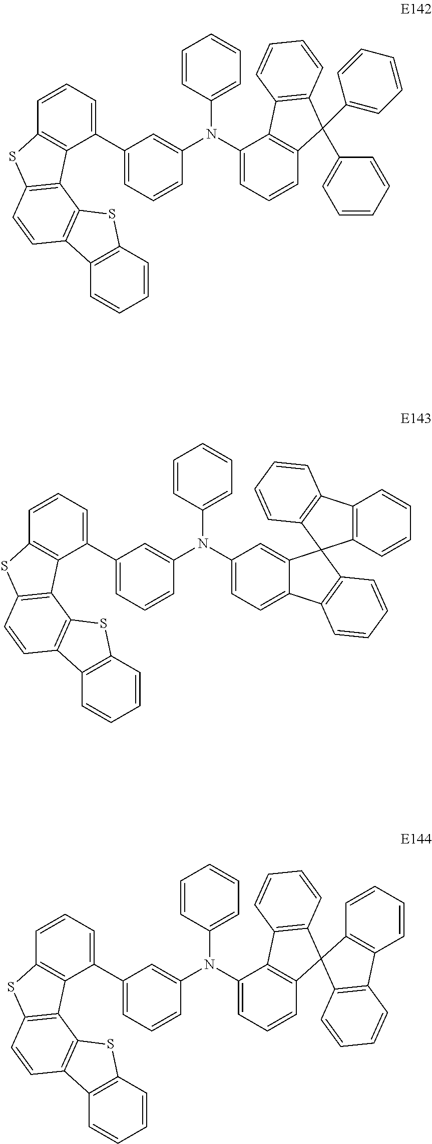 [SMILES: c1ccc(N(c2cccc(-c3cccc4sc5ccc6c7ccccc7sc6c5c34)c2)c2cccc3c2-c2ccccc2C3(c2ccccc2)c2ccccc2)cc1 ; c1ccc(N(c2cccc(-c3cccc4sc5ccc6c7ccccc7sc6c5c34)c2)c2ccc3c(c2)C2(c4ccccc4-c4ccccc42)c2ccccc2-3)cc1 ; c1ccc(N(c2cccc(-c3cccc4sc5ccc6c7ccccc7sc6c5c34)c2)c2cccc3c2-c2ccccc2C32c3ccccc3-c3ccccc32)cc1]

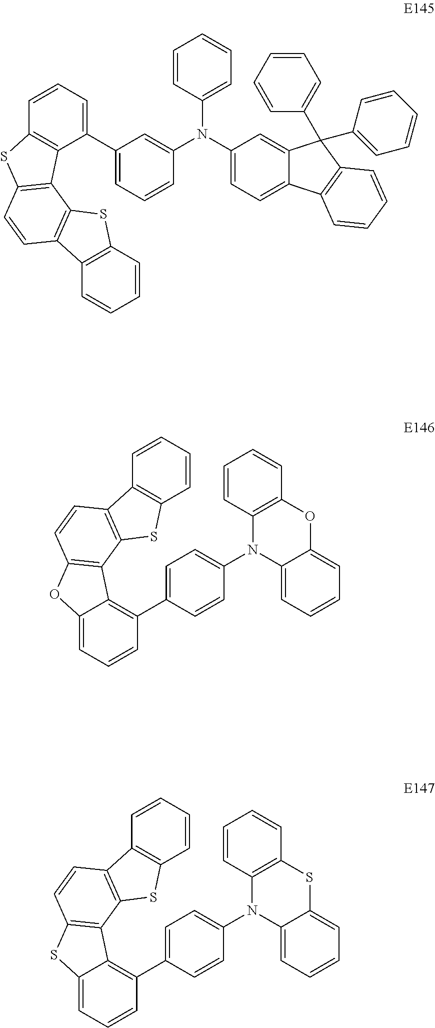 [SMILES: c1ccc(N(c2cccc(-c3cccc4sc5ccc6c7ccccc7sc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3oc4ccc5c6ccccc6sc5c4c23)cc1 ; c1ccc2c(c1)Sc1ccccc1N2c1ccc(-c2cccc3sc4ccc5c6ccccc6sc5c4c23)cc1]

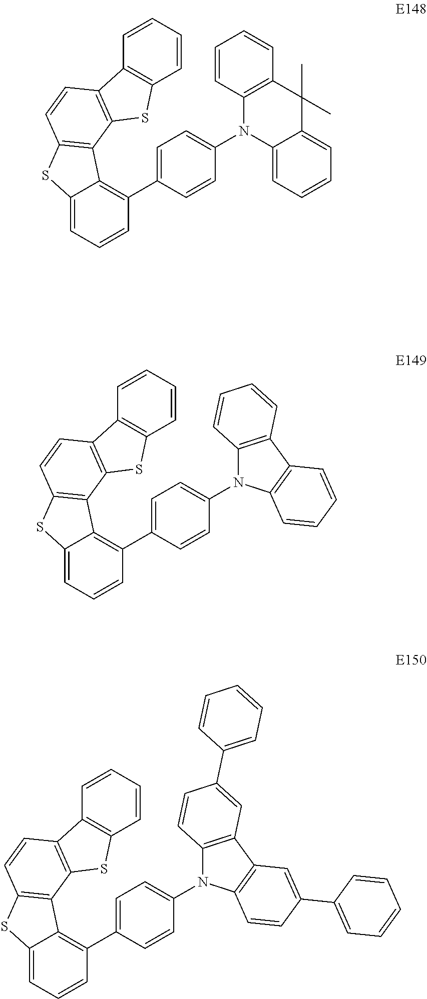 [SMILES: CC1(C)c2ccccc2N(c2ccc(-c3cccc4sc5ccc6c7ccccc7sc6c5c34)cc2)c2ccccc21 ; c1ccc2c(c1)sc1c2ccc2sc3cccc(-c4ccc(-n5c6ccccc6c6ccccc65)cc4)c3c21 ; c1ccc(-c2ccc3c(c2)c2cc(-c4ccccc4)ccc2n3-c2ccc(-c3cccc4sc5ccc6c7ccccc7sc6c5c34)cc2)cc1]

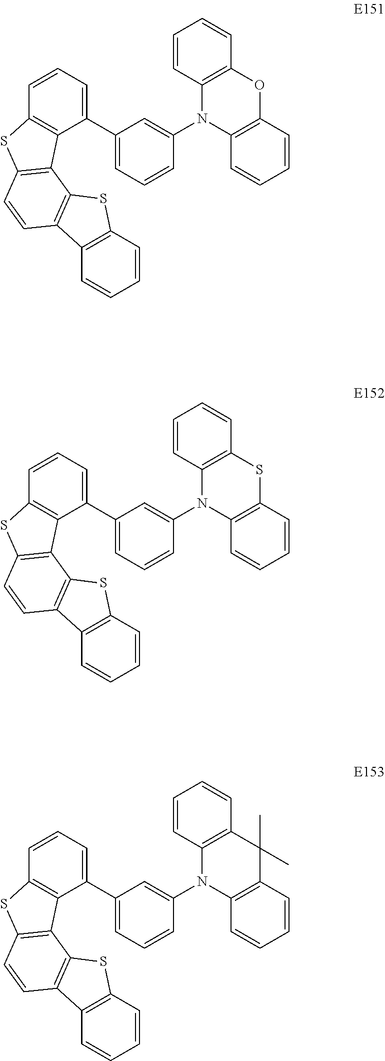 [SMILES: c1cc(-c2cccc3sc4ccc5c6ccccc6sc5c4c23)cc(N2c3ccccc3Oc3ccccc32)c1 ; c1cc(-c2cccc3sc4ccc5c6ccccc6sc5c4c23)cc(N2c3ccccc3Sc3ccccc32)c1 ; CC1(C)c2ccccc2N(c2cccc(-c3cccc4sc5ccc6c7ccccc7sc6c5c34)c2)c2ccccc21]

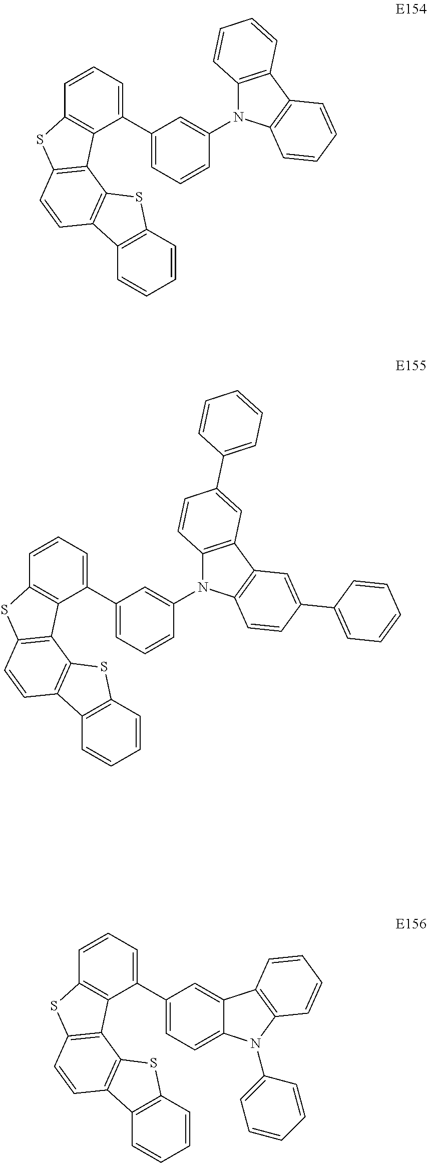 [SMILES: c1cc(-c2cccc3sc4ccc5c6ccccc6sc5c4c23)cc(-n2c3ccccc3c3ccccc32)c1 ; c1ccc(-c2ccc3c(c2)c2cc(-c4ccccc4)ccc2n3-c2cccc(-c3cccc4sc5ccc6c7ccccc7sc6c5c34)c2)cc1 ; c1ccc(-n2c3ccccc3c3cc(-c4cccc5sc6ccc7c8ccccc8sc7c6c45)ccc32)cc1]

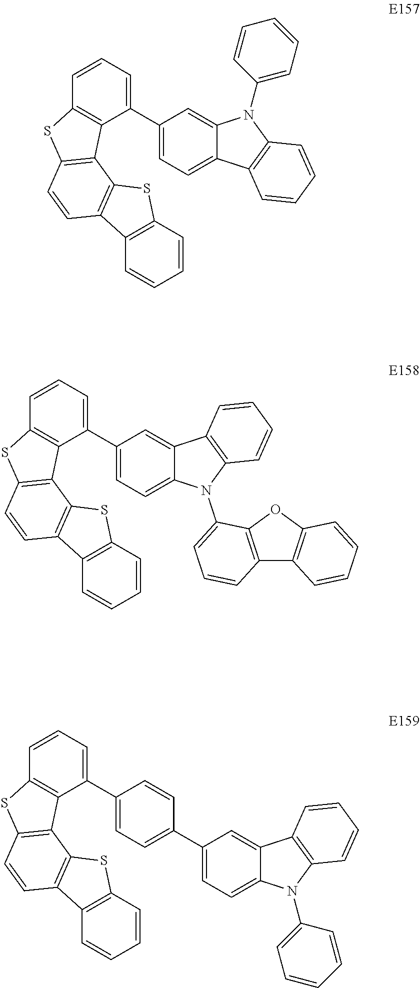 [SMILES: c1ccc(-n2c3ccccc3c3ccc(-c4cccc5sc6ccc7c8ccccc8sc7c6c45)cc32)cc1 ; c1ccc2c(c1)oc1c(-n3c4ccccc4c4cc(-c5cccc6sc7ccc8c9ccccc9sc8c7c56)ccc43)cccc12 ; c1ccc(-n2c3ccccc3c3cc(-c4ccc(-c5cccc6sc7ccc8c9ccccc9sc8c7c56)cc4)ccc32)cc1]

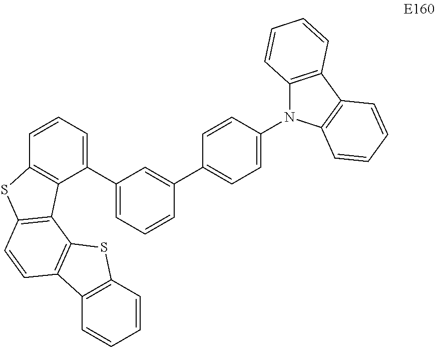 [SMILES: c1cc(-c2ccc(-n3c4ccccc4c4ccccc43)cc2)cc(-c2cccc3sc4ccc5c6ccccc6sc5c4c23)c1]

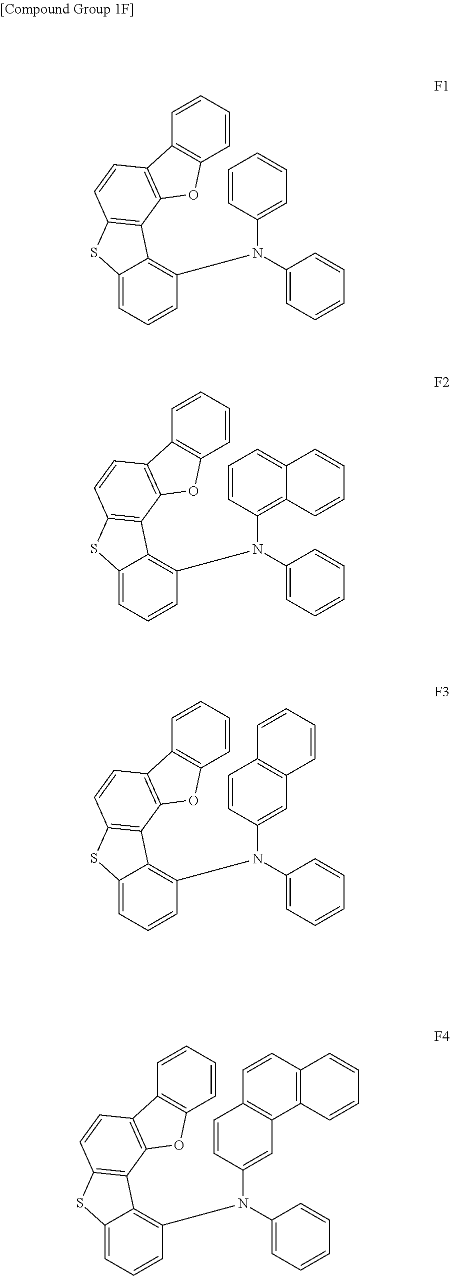 [SMILES: c1ccc(N(c2ccccc2)c2cccc3sc4ccc5c6ccccc6oc5c4c23)cc1 ; c1ccc(N(c2cccc3ccccc23)c2cccc3sc4ccc5c6ccccc6oc5c4c23)cc1 ; c1ccc(N(c2ccc3ccccc3c2)c2cccc3sc4ccc5c6ccccc6oc5c4c23)cc1 ; c1ccc(N(c2ccc3ccc4ccccc4c3c2)c2cccc3sc4ccc5c6ccccc6oc5c4c23)cc1]

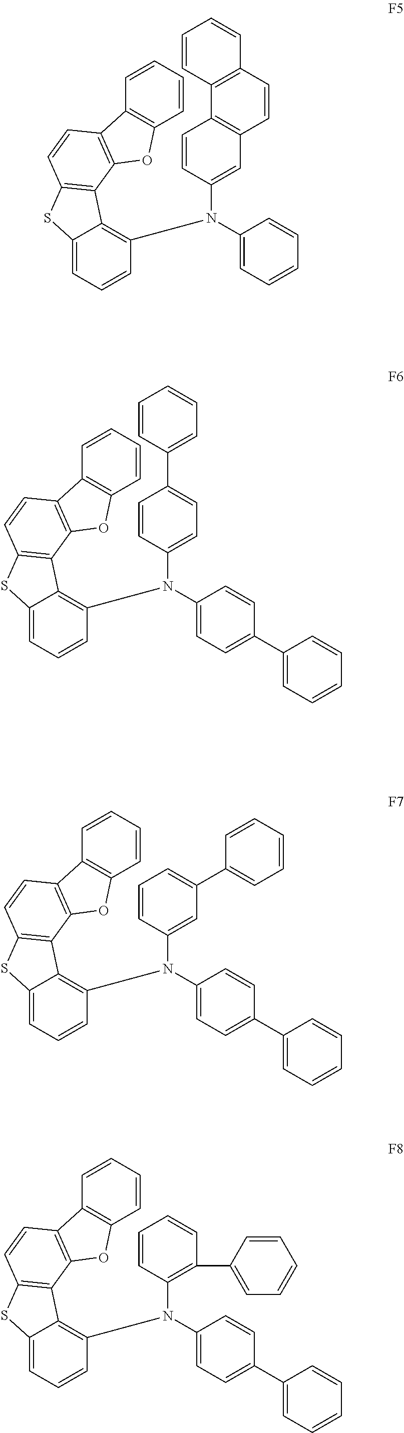 [SMILES: c1ccc(N(c2ccc3c(ccc4ccccc43)c2)c2cccc3sc4ccc5c6ccccc6oc5c4c23)cc1 ; c1ccc(-c2ccc(N(c3ccc(-c4ccccc4)cc3)c3cccc4sc5ccc6c7ccccc7oc6c5c34)cc2)cc1 ; c1ccc(-c2ccc(N(c3cccc(-c4ccccc4)c3)c3cccc4sc5ccc6c7ccccc7oc6c5c34)cc2)cc1 ; c1ccc(-c2ccc(N(c3ccccc3-c3ccccc3)c3cccc4sc5ccc6c7ccccc7oc6c5c34)cc2)cc1]

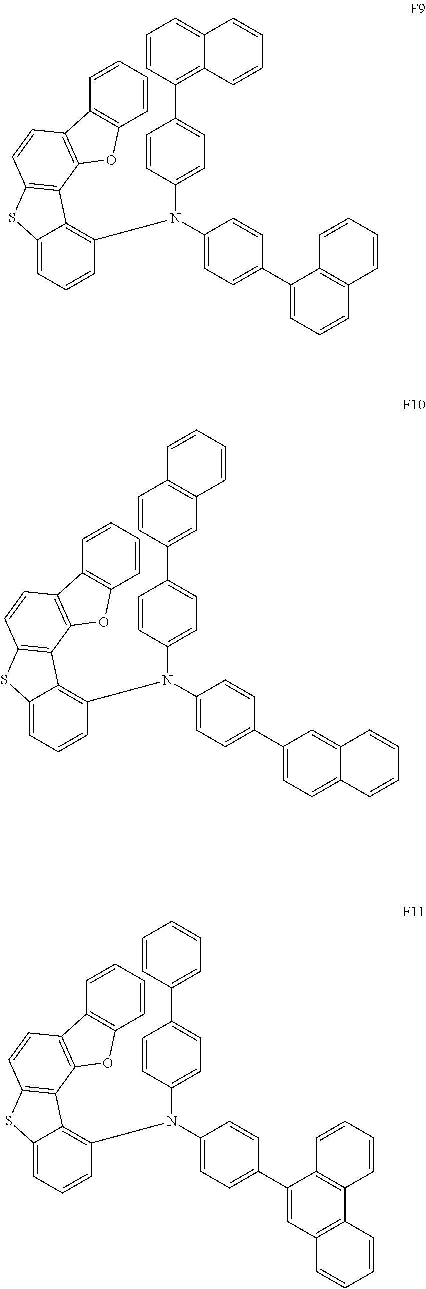 [SMILES: c1ccc2c(-c3ccc(N(c4ccc(-c5cccc6ccccc56)cc4)c4cccc5sc6ccc7c8ccccc8oc7c6c45)cc3)cccc2c1 ; c1ccc2cc(-c3ccc(N(c4ccc(-c5ccc6ccccc6c5)cc4)c4cccc5sc6ccc7c8ccccc8oc7c6c45)cc3)ccc2c1 ; c1ccc(-c2ccc(N(c3ccc(-c4cc5ccccc5c5ccccc45)cc3)c3cccc4sc5ccc6c7ccccc7oc6c5c34)cc2)cc1]

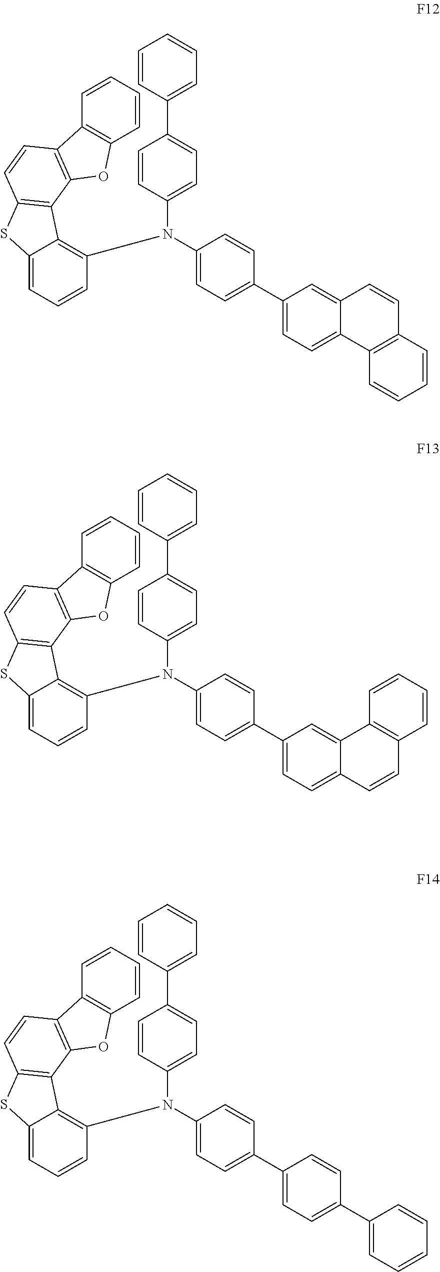 [SMILES: c1ccc(-c2ccc(N(c3ccc(-c4ccc5c(ccc6ccccc65)c4)cc3)c3cccc4sc5ccc6c7ccccc7oc6c5c34)cc2)cc1 ; c1ccc(-c2ccc(N(c3ccc(-c4ccc5ccc6ccccc6c5c4)cc3)c3cccc4sc5ccc6c7ccccc7oc6c5c34)cc2)cc1 ; c1ccc(-c2ccc(-c3ccc(N(c4ccc(-c5ccccc5)cc4)c4cccc5sc6ccc7c8ccccc8oc7c6c45)cc3)cc2)cc1]

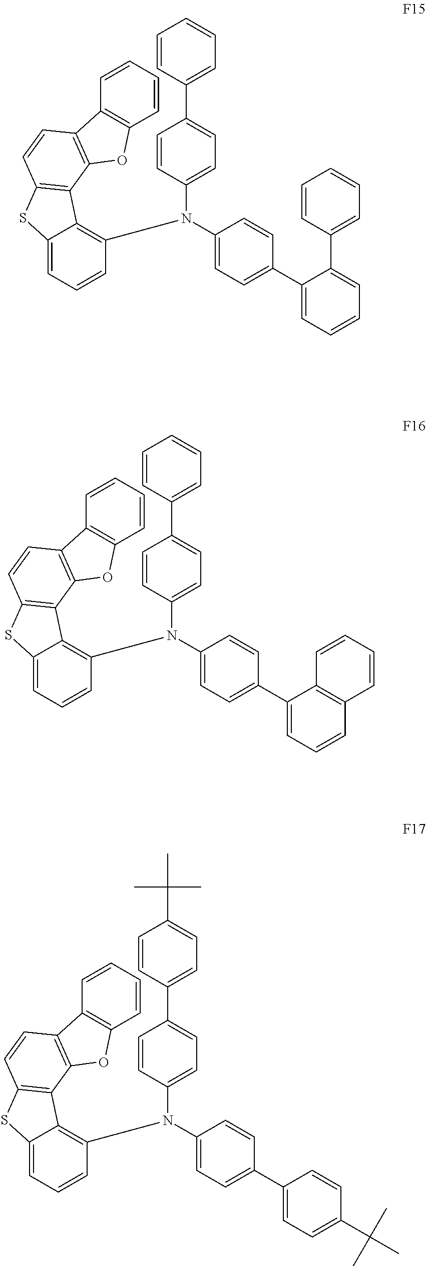 [SMILES: c1ccc(-c2ccc(N(c3ccc(-c4ccccc4-c4ccccc4)cc3)c3cccc4sc5ccc6c7ccccc7oc6c5c34)cc2)cc1 ; c1ccc(-c2ccc(N(c3ccc(-c4cccc5ccccc45)cc3)c3cccc4sc5ccc6c7ccccc7oc6c5c34)cc2)cc1 ; CC(C)(C)c1ccc(-c2ccc(N(c3ccc(-c4ccc(C(C)(C)C)cc4)cc3)c3cccc4sc5ccc6c7ccccc7oc6c5c34)cc2)cc1]

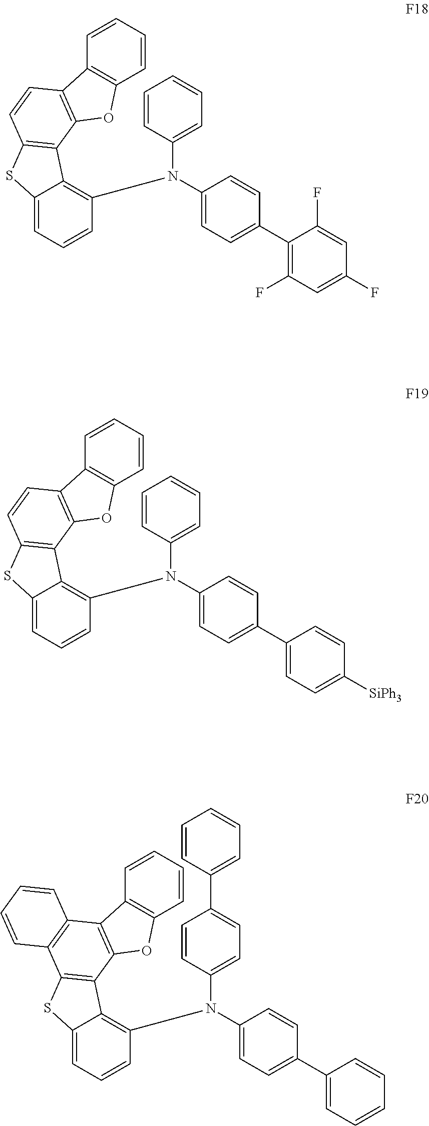

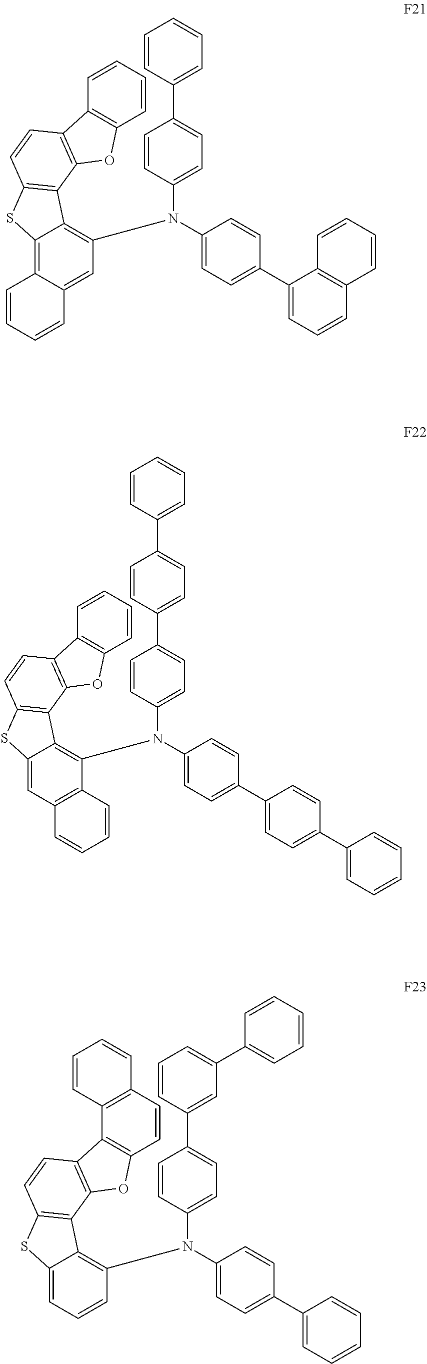 [SMILES: c1ccc(-c2ccc(N(c3ccc(-c4cccc5ccccc45)cc3)c3cc4ccccc4c4sc5ccc6c7ccccc7oc6c5c34)cc2)cc1 ; c1ccc(-c2ccc(-c3ccc(N(c4ccc(-c5ccc(-c6ccccc6)cc5)cc4)c4c5ccccc5cc5sc6ccc7c8ccccc8oc7c6c45)cc3)cc2)cc1 ; c1ccc(-c2ccc(N(c3ccc(-c4cccc(-c5ccccc5)c4)cc3)c3cccc4sc5ccc6c(oc7ccc8ccccc8c76)c5c34)cc2)cc1]

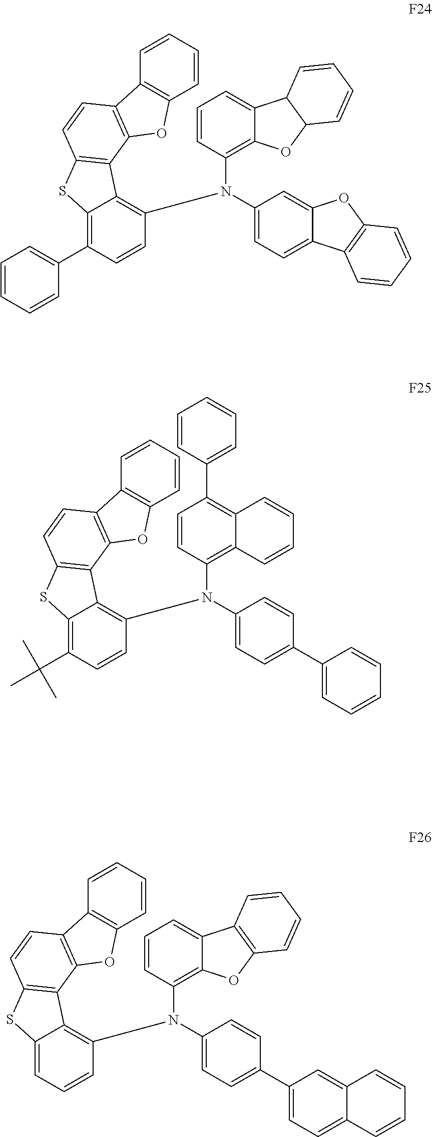 [SMILES: C1=CC2Oc3c(cccc3N(c3ccc4c(c3)oc3ccccc34)c3ccc(-c4ccccc4)c4sc5ccc6c7ccccc7oc6c5c34)C2C=C1 ; CC(C)(C)c1ccc(N(c2ccc(-c3ccccc3)cc2)c2ccc(-c3ccccc3)c3ccccc23)c2c1sc1ccc3c4ccccc4oc3c12 ; c1ccc2cc(-c3ccc(N(c4cccc5c4oc4ccccc45)c4cccc5sc6ccc7c8ccccc8oc7c6c45)cc3)ccc2c1]

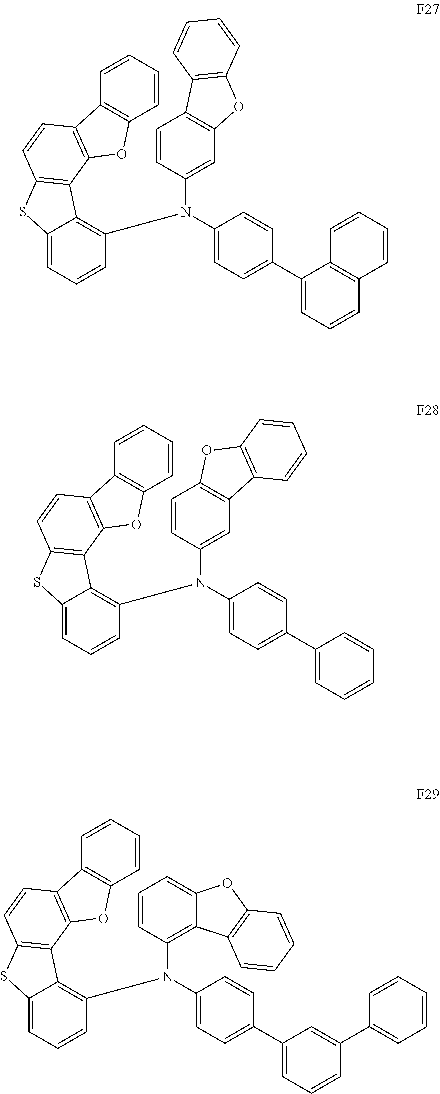 [SMILES: c1ccc2c(-c3ccc(N(c4ccc5c(c4)oc4ccccc45)c4cccc5sc6ccc7c8ccccc8oc7c6c45)cc3)cccc2c1 ; c1ccc(-c2ccc(N(c3ccc4oc5ccccc5c4c3)c3cccc4sc5ccc6c7ccccc7oc6c5c34)cc2)cc1 ; c1ccc(-c2cccc(-c3ccc(N(c4cccc5oc6ccccc6c45)c4cccc5sc6ccc7c8ccccc8oc7c6c45)cc3)c2)cc1]

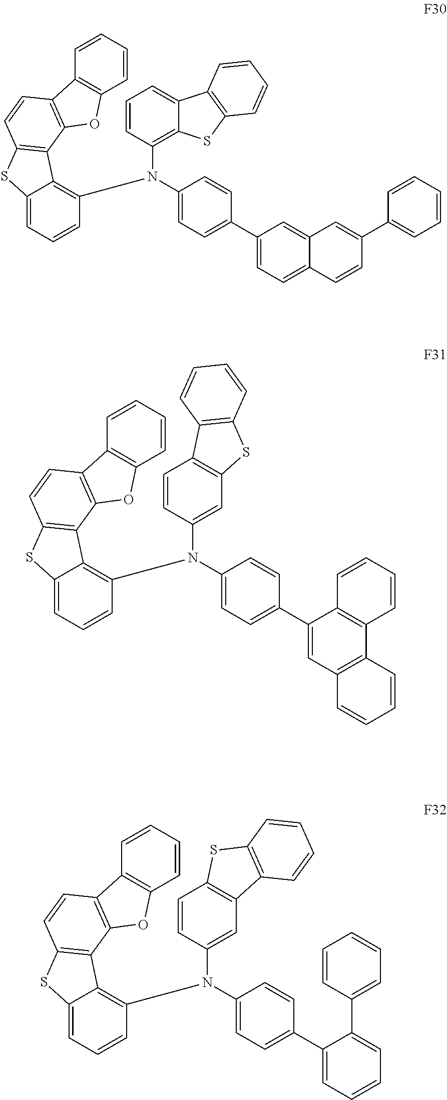 [SMILES: c1ccc(-c2ccc3ccc(-c4ccc(N(c5cccc6c5sc5ccccc56)c5cccc6sc7ccc8c9ccccc9oc8c7c56)cc4)cc3c2)cc1 ; c1ccc2c(c1)cc(-c1ccc(N(c3ccc4c(c3)sc3ccccc34)c3cccc4sc5ccc6c7ccccc7oc6c5c34)cc1)c1ccccc12 ; c1ccc(-c2ccccc2-c2ccc(N(c3ccc4sc5ccccc5c4c3)c3cccc4sc5ccc6c7ccccc7oc6c5c34)cc2)cc1]

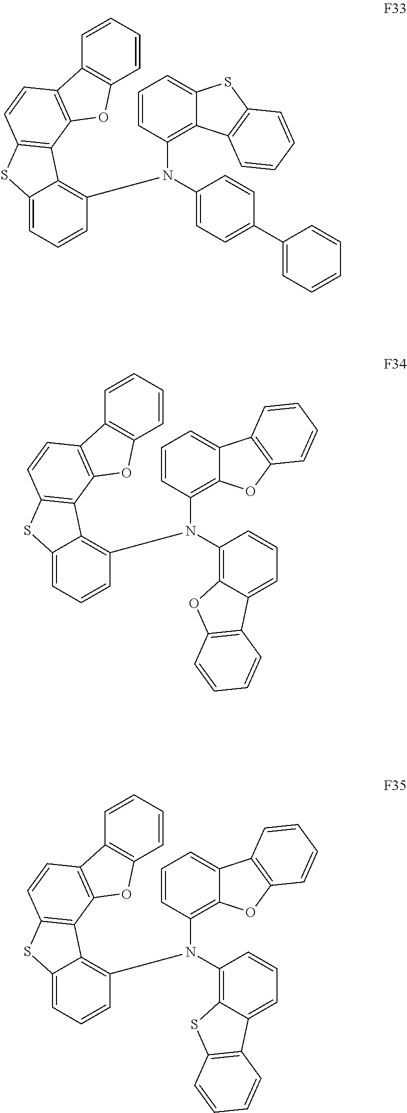 [SMILES: c1ccc(-c2ccc(N(c3cccc4sc5ccccc5c34)c3cccc4sc5ccc6c7ccccc7oc6c5c34)cc2)cc1 ; c1ccc2c(c1)oc1c(N(c3cccc4c3oc3ccccc34)c3cccc4sc5ccc6c7ccccc7oc6c5c34)cccc12 ; c1ccc2c(c1)oc1c(N(c3cccc4c3sc3ccccc34)c3cccc4sc5ccc6c7ccccc7oc6c5c34)cccc12]

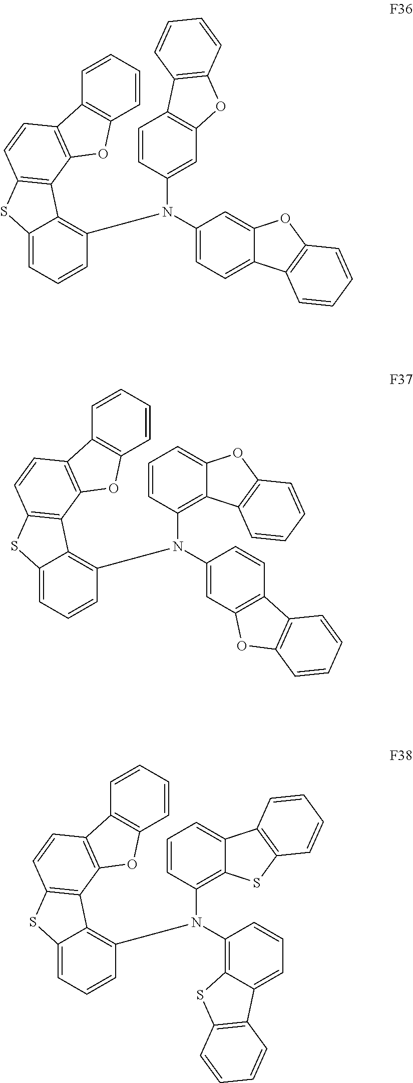 [SMILES: c1ccc2c(c1)oc1cc(N(c3ccc4c(c3)oc3ccccc34)c3cccc4sc5ccc6c7ccccc7oc6c5c34)ccc12 ; c1ccc2c(c1)oc1cc(N(c3cccc4oc5ccccc5c34)c3cccc4sc5ccc6c7ccccc7oc6c5c34)ccc12 ; c1ccc2c(c1)oc1c2ccc2sc3cccc(N(c4cccc5c4sc4ccccc45)c4cccc5c4sc4ccccc45)c3c21]

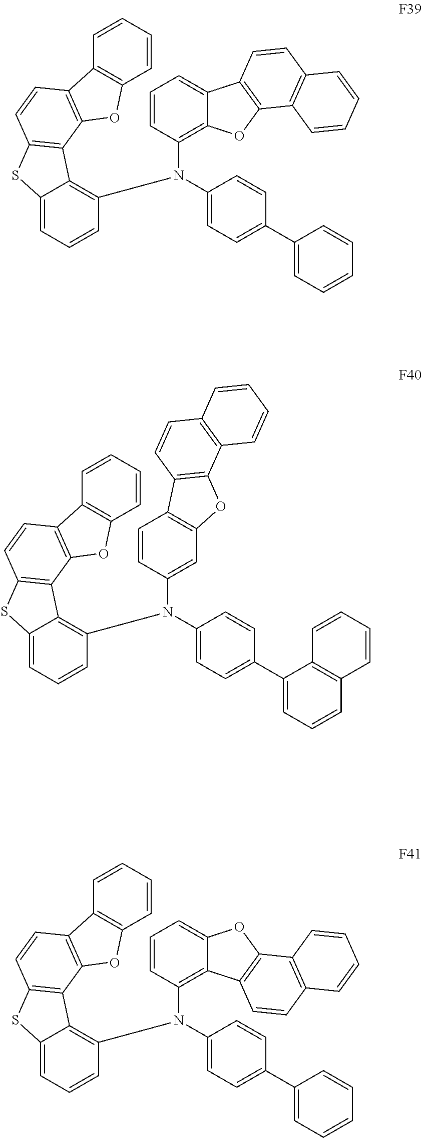 [SMILES: c1ccc(-c2ccc(N(c3cccc4c3oc3c5ccccc5ccc43)c3cccc4sc5ccc6c7ccccc7oc6c5c34)cc2)cc1 ; c1ccc2c(-c3ccc(N(c4ccc5c(c4)oc4c6ccccc6ccc54)c4cccc5sc6ccc7c8ccccc8oc7c6c45)cc3)cccc2c1 ; c1ccc(-c2ccc(N(c3cccc4oc5c6ccccc6ccc5c34)c3cccc4sc5ccc6c7ccccc7oc6c5c34)cc2)cc1]

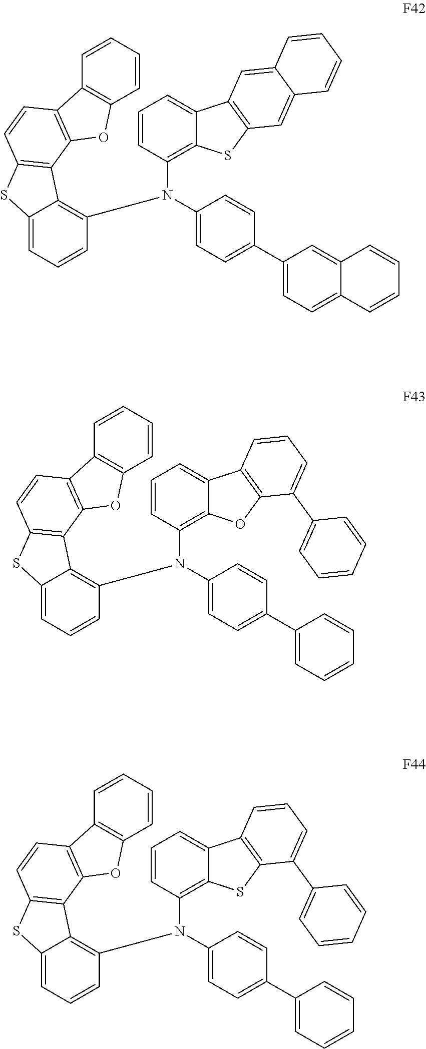 [SMILES: c1ccc2cc(-c3ccc(N(c4cccc5c4sc4cc6ccccc6cc45)c4cccc5sc6ccc7c8ccccc8oc7c6c45)cc3)ccc2c1 ; c1ccc(-c2ccc(N(c3cccc4c3oc3c(-c5ccccc5)cccc34)c3cccc4sc5ccc6c7ccccc7oc6c5c34)cc2)cc1 ; c1ccc(-c2ccc(N(c3cccc4c3sc3c(-c5ccccc5)cccc34)c3cccc4sc5ccc6c7ccccc7oc6c5c34)cc2)cc1]

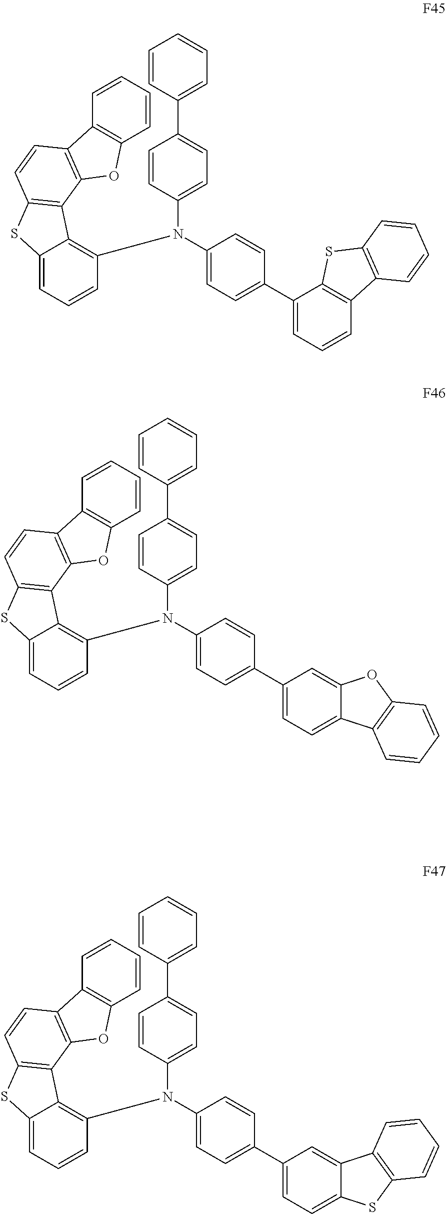 [SMILES: c1ccc(-c2ccc(N(c3ccc(-c4cccc5c4sc4ccccc45)cc3)c3cccc4sc5ccc6c7ccccc7oc6c5c34)cc2)cc1 ; c1ccc(-c2ccc(N(c3ccc(-c4ccc5c(c4)oc4ccccc45)cc3)c3cccc4sc5ccc6c7ccccc7oc6c5c34)cc2)cc1 ; c1ccc(-c2ccc(N(c3ccc(-c4ccc5sc6ccccc6c5c4)cc3)c3cccc4sc5ccc6c7ccccc7oc6c5c34)cc2)cc1]

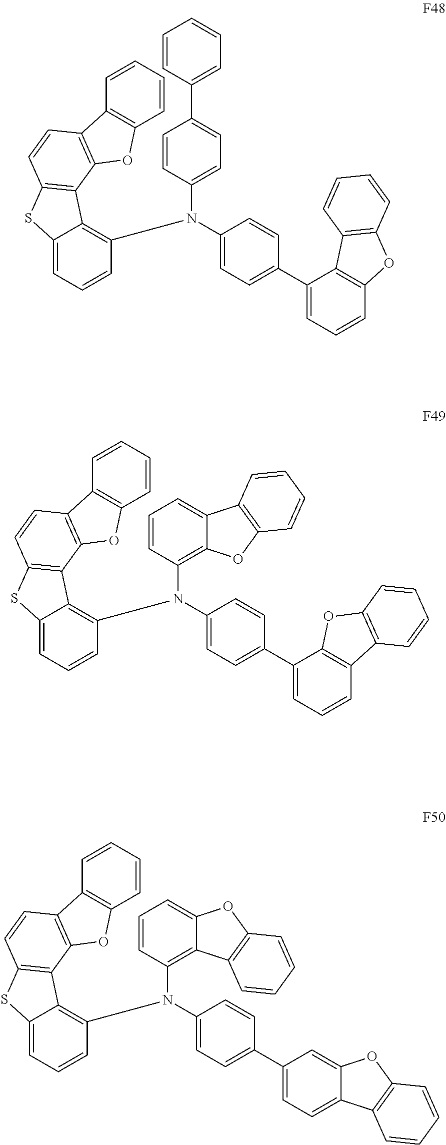 [SMILES: c1ccc(-c2ccc(N(c3ccc(-c4cccc5oc6ccccc6c45)cc3)c3cccc4sc5ccc6c7ccccc7oc6c5c34)cc2)cc1 ; c1ccc2c(c1)oc1c(-c3ccc(N(c4cccc5c4oc4ccccc45)c4cccc5sc6ccc7c8ccccc8oc7c6c45)cc3)cccc12 ; c1ccc2c(c1)oc1cc(-c3ccc(N(c4cccc5oc6ccccc6c45)c4cccc5sc6ccc7c8ccccc8oc7c6c45)cc3)ccc12]

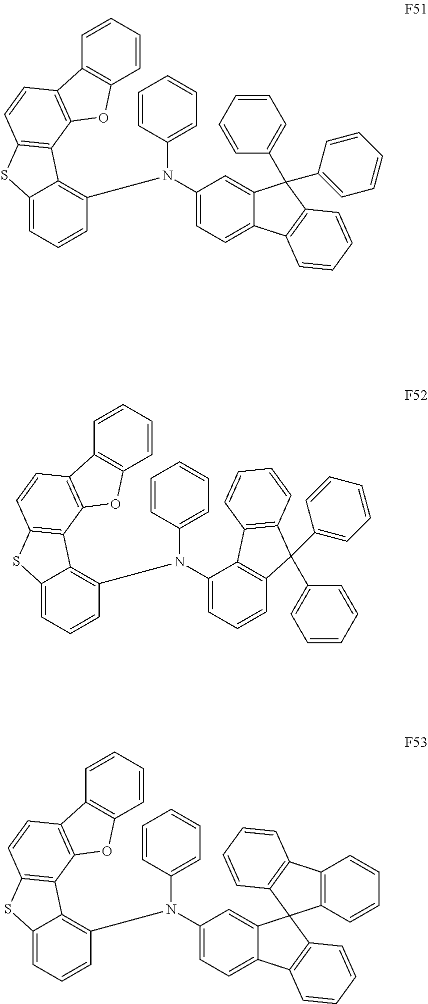 [SMILES: c1ccc(N(c2ccc3c(c2)C(c2ccccc2)(c2ccccc2)c2ccccc2-3)c2cccc3sc4ccc5c6ccccc6oc5c4c23)cc1 ; c1ccc(N(c2cccc3c2-c2ccccc2C3(c2ccccc2)c2ccccc2)c2cccc3sc4ccc5c6ccccc6oc5c4c23)cc1 ; c1ccc(N(c2ccc3c(c2)C2(c4ccccc4-c4ccccc42)c2ccccc2-3)c2cccc3sc4ccc5c6ccccc6oc5c4c23)cc1]

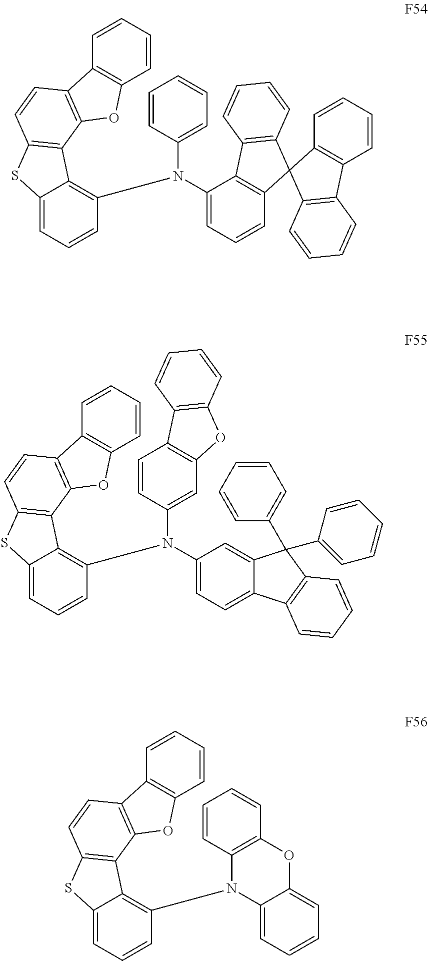 [SMILES: c1ccc(N(c2cccc3c2-c2ccccc2C32c3ccccc3-c3ccccc32)c2cccc3sc4ccc5c6ccccc6oc5c4c23)cc1 ; c1ccc(C2(c3ccccc3)c3ccccc3-c3ccc(N(c4ccc5c(c4)oc4ccccc45)c4cccc5sc6ccc7c8ccccc8oc7c6c45)cc32)cc1 ; c1ccc2c(c1)Oc1ccccc1N2c1cccc2sc3ccc4c5ccccc5oc4c3c12]

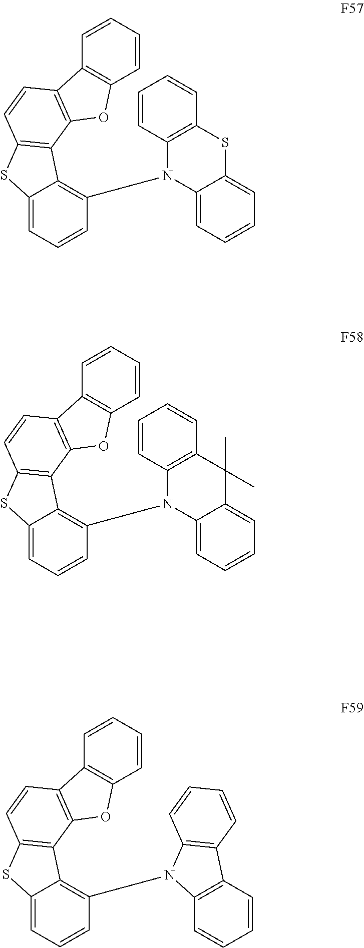 [SMILES: c1ccc2c(c1)Sc1ccccc1N2c1cccc2sc3ccc4c5ccccc5oc4c3c12 ; CC1(C)c2ccccc2N(c2cccc3sc4ccc5c6ccccc6oc5c4c23)c2ccccc21 ; c1ccc2c(c1)oc1c2ccc2sc3cccc(-n4c5ccccc5c5ccccc54)c3c21]

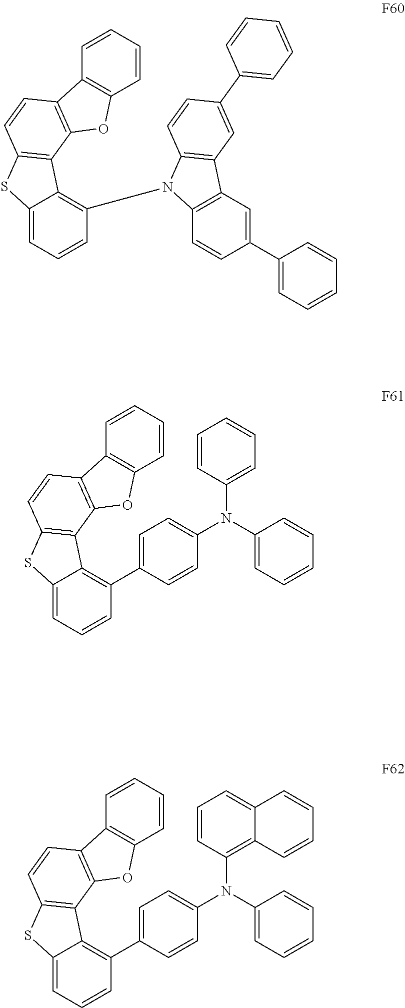 [SMILES: c1ccc(-c2ccc3c(c2)c2cc(-c4ccccc4)ccc2n3-c2cccc3sc4ccc5c6ccccc6oc5c4c23)cc1 ; c1ccc(N(c2ccccc2)c2ccc(-c3cccc4sc5ccc6c7ccccc7oc6c5c34)cc2)cc1 ; c1ccc(N(c2ccc(-c3cccc4sc5ccc6c7ccccc7oc6c5c34)cc2)c2cccc3ccccc23)cc1]

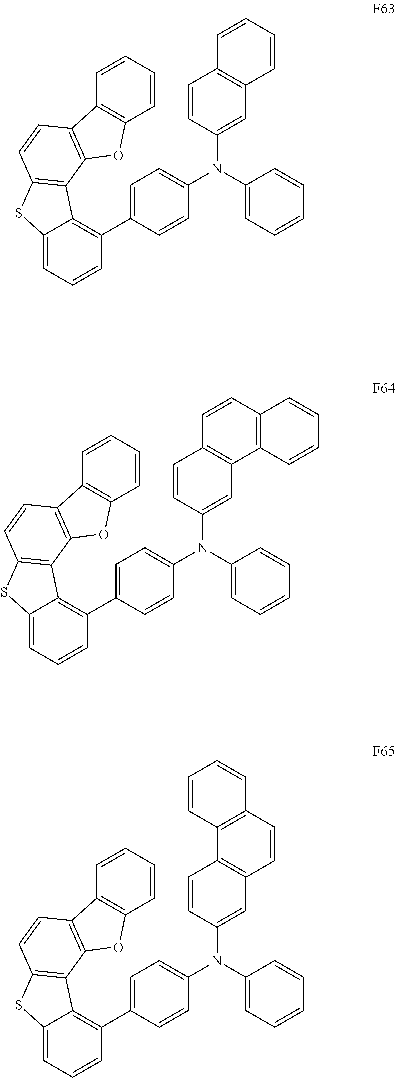 [SMILES: c1ccc(N(c2ccc(-c3cccc4sc5ccc6c7ccccc7oc6c5c34)cc2)c2ccc3ccccc3c2)cc1 ; c1ccc(N(c2ccc(-c3cccc4sc5ccc6c7ccccc7oc6c5c34)cc2)c2ccc3ccc4ccccc4c3c2)cc1 ; c1ccc(N(c2ccc(-c3cccc4sc5ccc6c7ccccc7oc6c5c34)cc2)c2ccc3c(ccc4ccccc43)c2)cc1]

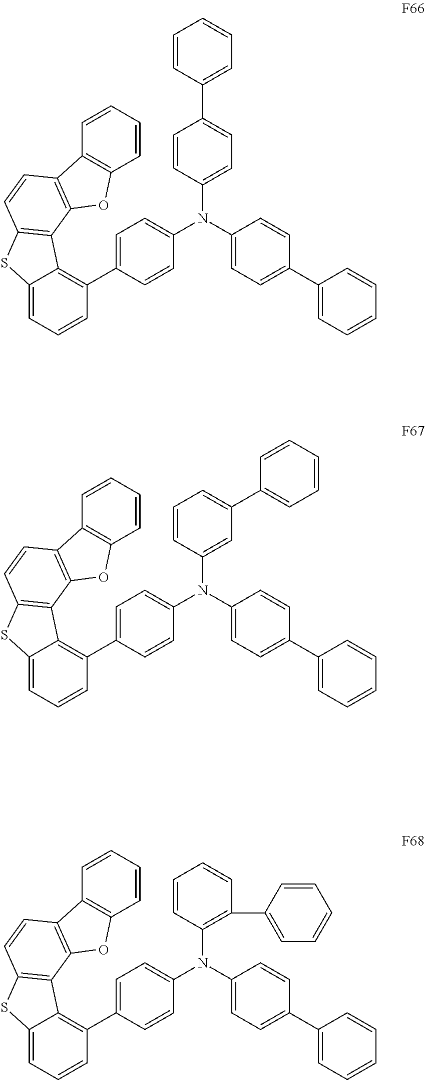 [SMILES: c1ccc(-c2ccc(N(c3ccc(-c4ccccc4)cc3)c3ccc(-c4cccc5sc6ccc7c8ccccc8oc7c6c45)cc3)cc2)cc1 ; c1ccc(-c2ccc(N(c3ccc(-c4cccc5sc6ccc7c8ccccc8oc7c6c45)cc3)c3cccc(-c4ccccc4)c3)cc2)cc1 ; c1ccc(-c2ccc(N(c3ccc(-c4cccc5sc6ccc7c8ccccc8oc7c6c45)cc3)c3ccccc3-c3ccccc3)cc2)cc1]

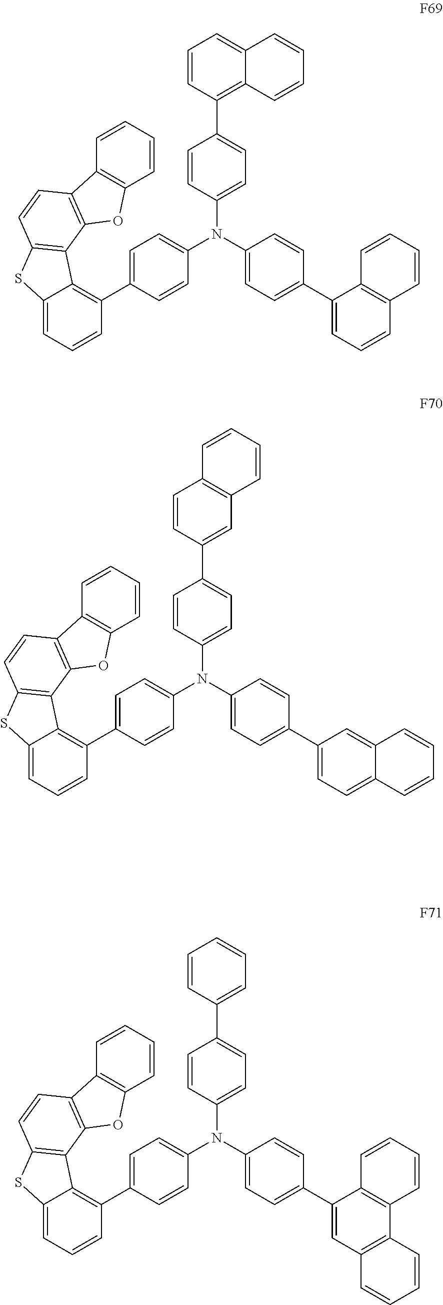 [SMILES: c1ccc2c(-c3ccc(N(c4ccc(-c5cccc6ccccc56)cc4)c4ccc(-c5cccc6sc7ccc8c9ccccc9oc8c7c56)cc4)cc3)cccc2c1 ; c1ccc2cc(-c3ccc(N(c4ccc(-c5ccc6ccccc6c5)cc4)c4ccc(-c5cccc6sc7ccc8c9ccccc9oc8c7c56)cc4)cc3)ccc2c1 ; c1ccc(-c2ccc(N(c3ccc(-c4cc5ccccc5c5ccccc45)cc3)c3ccc(-c4cccc5sc6ccc7c8ccccc8oc7c6c45)cc3)cc2)cc1]

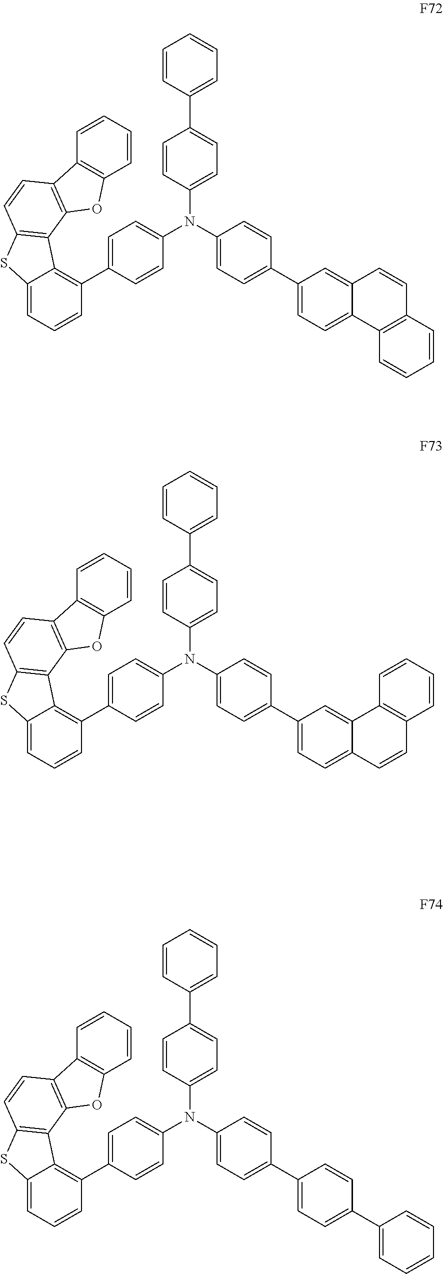 [SMILES: c1ccc(-c2ccc(N(c3ccc(-c4ccc5c(ccc6ccccc65)c4)cc3)c3ccc(-c4cccc5sc6ccc7c8ccccc8oc7c6c45)cc3)cc2)cc1 ; c1ccc(-c2ccc(N(c3ccc(-c4ccc5ccc6ccccc6c5c4)cc3)c3ccc(-c4cccc5sc6ccc7c8ccccc8oc7c6c45)cc3)cc2)cc1 ; c1ccc(-c2ccc(-c3ccc(N(c4ccc(-c5ccccc5)cc4)c4ccc(-c5cccc6sc7ccc8c9ccccc9oc8c7c56)cc4)cc3)cc2)cc1]

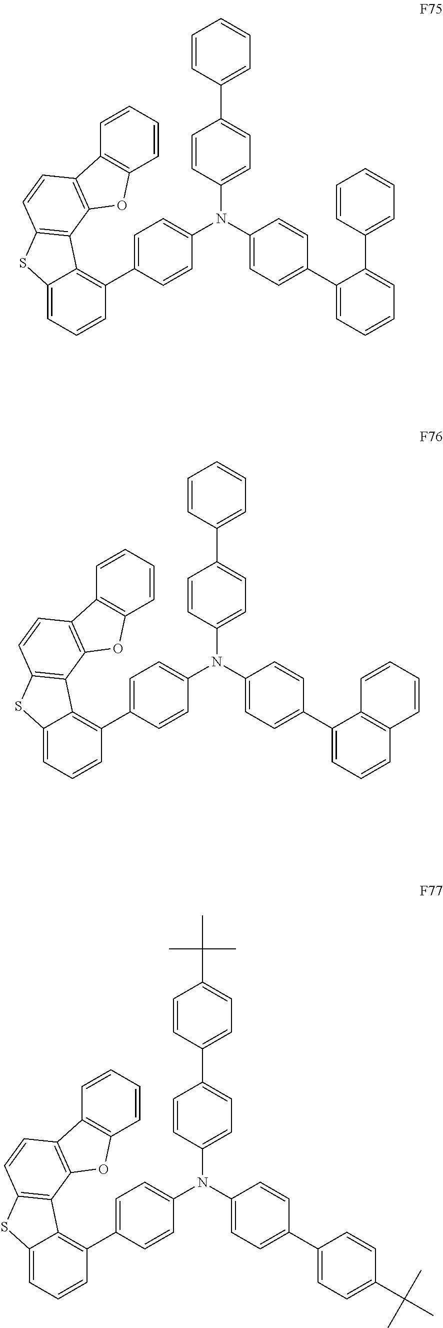 [SMILES: c1ccc(-c2ccc(N(c3ccc(-c4ccccc4-c4ccccc4)cc3)c3ccc(-c4cccc5sc6ccc7c8ccccc8oc7c6c45)cc3)cc2)cc1 ; c1ccc(-c2ccc(N(c3ccc(-c4cccc5ccccc45)cc3)c3ccc(-c4cccc5sc6ccc7c8ccccc8oc7c6c45)cc3)cc2)cc1 ; CC(C)(C)c1ccc(-c2ccc(N(c3ccc(-c4ccc(C(C)(C)C)cc4)cc3)c3ccc(-c4cccc5sc6ccc7c8ccccc8oc7c6c45)cc3)cc2)cc1]

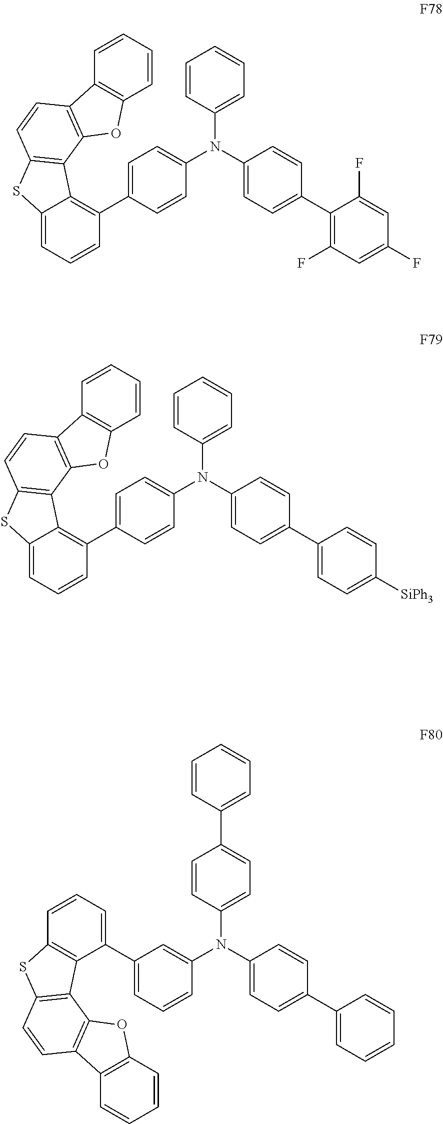

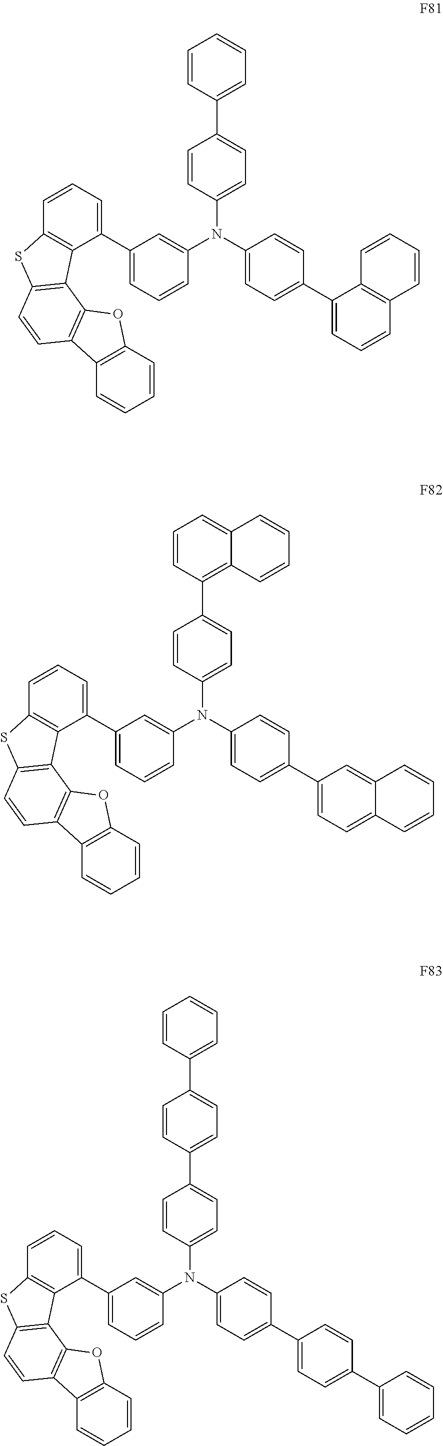 [SMILES: c1ccc(-c2ccc(N(c3ccc(-c4cccc5ccccc45)cc3)c3cccc(-c4cccc5sc6ccc7c8ccccc8oc7c6c45)c3)cc2)cc1 ; c1cc(-c2cccc3sc4ccc5c6ccccc6oc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6sc7ccc8c9ccccc9oc8c7c56)c4)cc3)cc2)cc1]

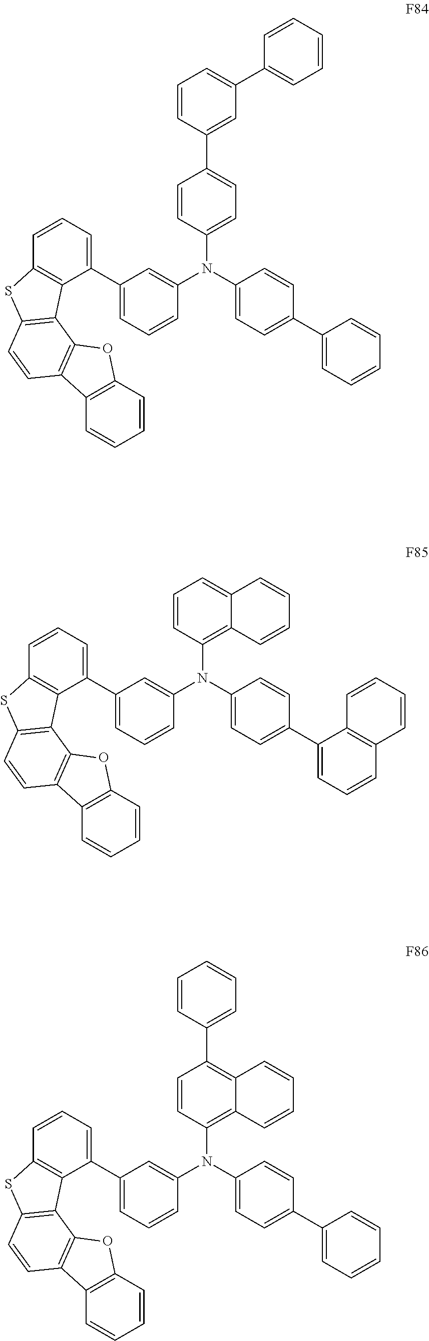 [SMILES: c1ccc(-c2ccc(N(c3ccc(-c4cccc(-c5ccccc5)c4)cc3)c3cccc(-c4cccc5sc6ccc7c8ccccc8oc7c6c45)c3)cc2)cc1 ; c1cc(-c2cccc3sc4ccc5c6ccccc6oc5c4c23)cc(N(c2ccc(-c3cccc4ccccc34)cc2)c2cccc3ccccc23)c1 ; c1ccc(-c2ccc(N(c3cccc(-c4cccc5sc6ccc7c8ccccc8oc7c6c45)c3)c3ccc(-c4ccccc4)c4ccccc34)cc2)cc1]

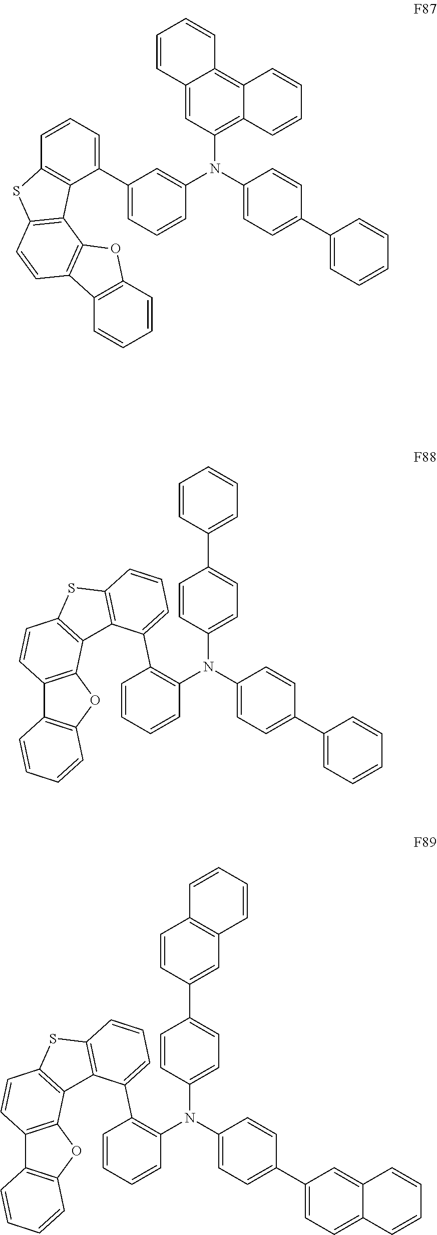 [SMILES: c1ccc(-c2ccc(N(c3cccc(-c4cccc5sc6ccc7c8ccccc8oc7c6c45)c3)c3cc4ccccc4c4ccccc34)cc2)cc1 ; c1ccc(-c2ccc(N(c3ccc(-c4ccccc4)cc3)c3ccccc3-c3cccc4sc5ccc6c7ccccc7oc6c5c34)cc2)cc1 ; c1ccc(N(c2ccc(-c3ccc4ccccc4c3)cc2)c2ccc(-c3ccc4ccccc4c3)cc2)c(-c2cccc3sc4ccc5c6ccccc6oc5c4c23)c1]

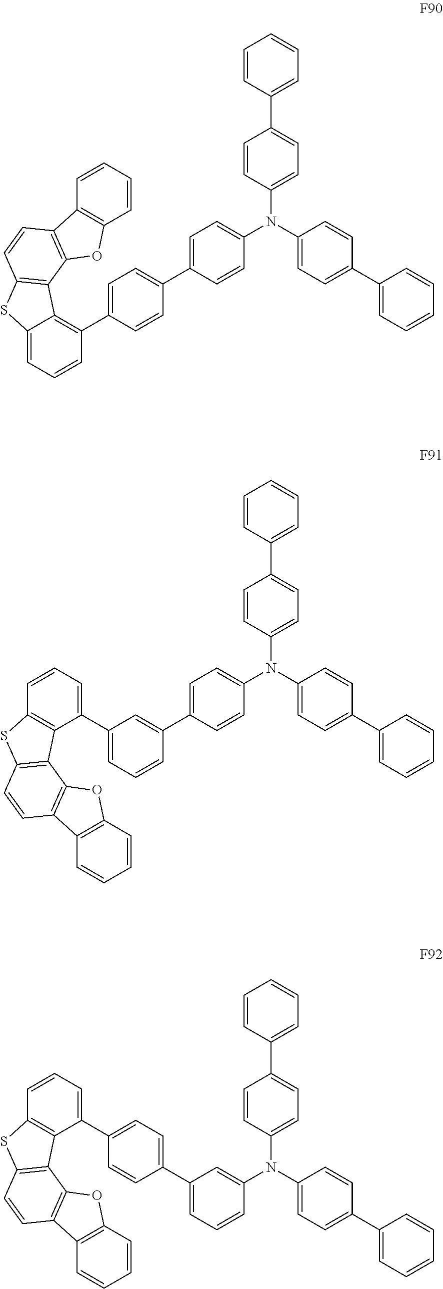 [SMILES: c1ccc(-c2ccc(N(c3ccc(-c4ccccc4)cc3)c3ccc(-c4ccc(-c5cccc6sc7ccc8c9ccccc9oc8c7c56)cc4)cc3)cc2)cc1 ; c1ccc(-c2ccc(N(c3ccc(-c4ccccc4)cc3)c3ccc(-c4cccc(-c5cccc6sc7ccc8c9ccccc9oc8c7c56)c4)cc3)cc2)cc1 ; c1ccc(-c2ccc(N(c3ccc(-c4ccccc4)cc3)c3cccc(-c4ccc(-c5cccc6sc7ccc8c9ccccc9oc8c7c56)cc4)c3)cc2)cc1]

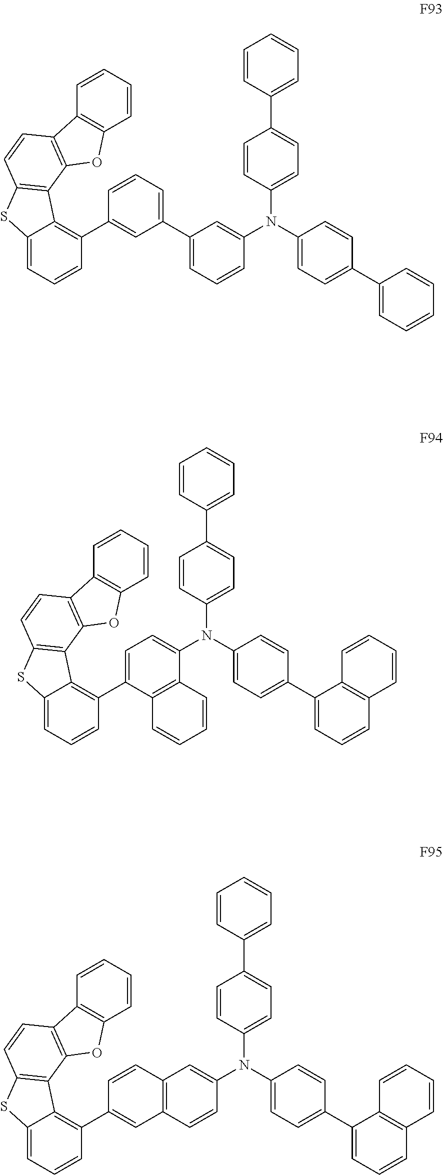 [SMILES: c1ccc(-c2ccc(N(c3ccc(-c4ccccc4)cc3)c3cccc(-c4cccc(-c5cccc6sc7ccc8c9ccccc9oc8c7c56)c4)c3)cc2)cc1 ; c1ccc(-c2ccc(N(c3ccc(-c4cccc5ccccc45)cc3)c3ccc(-c4cccc5sc6ccc7c8ccccc8oc7c6c45)c4ccccc34)cc2)cc1 ; c1ccc(-c2ccc(N(c3ccc(-c4cccc5ccccc45)cc3)c3ccc4cc(-c5cccc6sc7ccc8c9ccccc9oc8c7c56)ccc4c3)cc2)cc1]

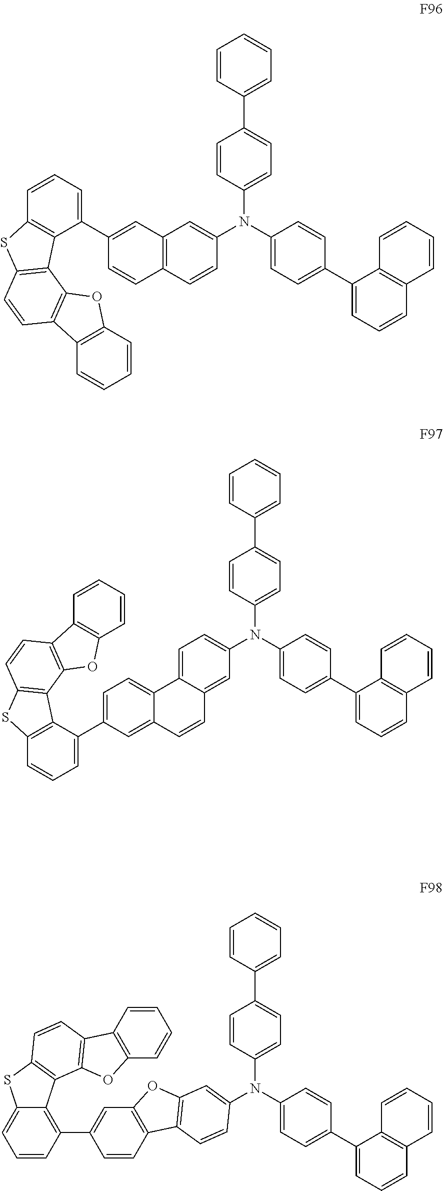 [SMILES: c1ccc(-c2ccc(N(c3ccc(-c4cccc5ccccc45)cc3)c3ccc4ccc(-c5cccc6sc7ccc8c9ccccc9oc8c7c56)cc4c3)cc2)cc1 ; c1ccc(-c2ccc(N(c3ccc(-c4cccc5ccccc45)cc3)c3ccc4c(ccc5cc(-c6cccc7sc8ccc9c%10ccccc%10oc9c8c67)ccc54)c3)cc2)cc1 ; c1ccc(-c2ccc(N(c3ccc(-c4cccc5ccccc45)cc3)c3ccc4c(c3)oc3cc(-c5cccc6sc7ccc8c9ccccc9oc8c7c56)ccc34)cc2)cc1]

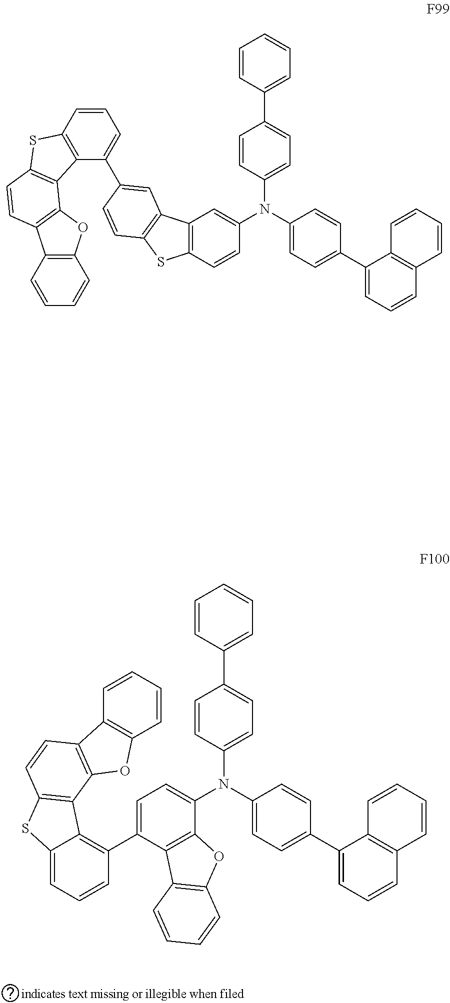 [SMILES: c1ccc(-c2ccc(N(c3ccc(-c4cccc5ccccc45)cc3)c3ccc4sc5ccc(-c6cccc7sc8ccc9c%10ccccc%10oc9c8c67)cc5c4c3)cc2)cc1 ; c1ccc(-c2ccc(N(c3ccc(-c4cccc5ccccc45)cc3)c3ccc(-c4cccc5sc6ccc7c8ccccc8oc7c6c45)c4c3oc3ccccc34)cc2)cc1]

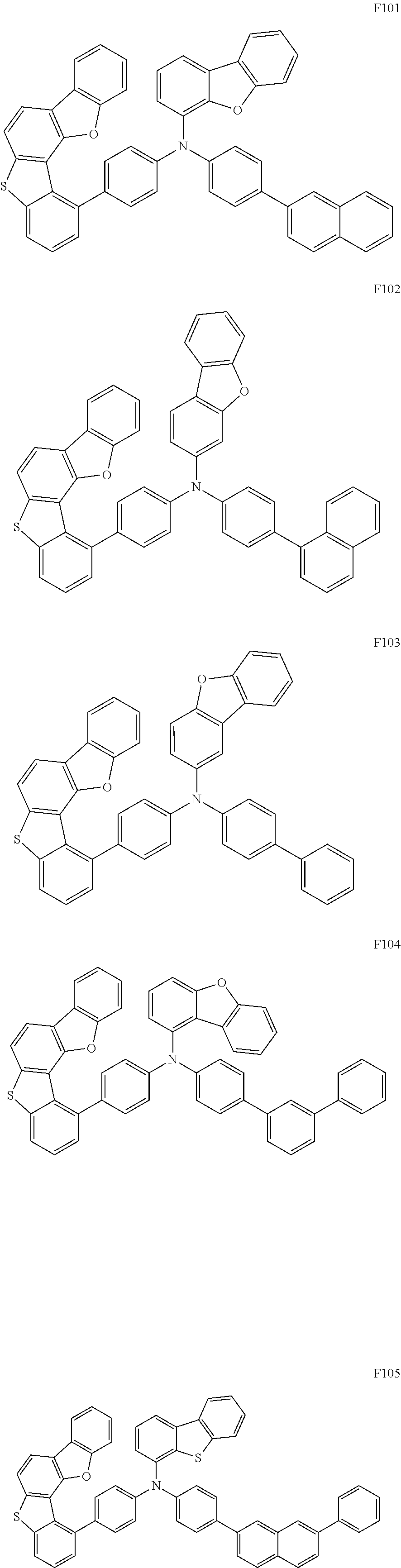 [SMILES: c1ccc2cc(-c3ccc(N(c4ccc(-c5cccc6sc7ccc8c9ccccc9oc8c7c56)cc4)c4cccc5c4oc4ccccc45)cc3)ccc2c1 ; c1ccc2c(-c3ccc(N(c4ccc(-c5cccc6sc7ccc8c9ccccc9oc8c7c56)cc4)c4ccc5c(c4)oc4ccccc45)cc3)cccc2c1 ; c1ccc(-c2ccc(N(c3ccc(-c4cccc5sc6ccc7c8ccccc8oc7c6c45)cc3)c3ccc4oc5ccccc5c4c3)cc2)cc1 ; c1ccc(-c2cccc(-c3ccc(N(c4ccc(-c5cccc6sc7ccc8c9ccccc9oc8c7c56)cc4)c4cccc5oc6ccccc6c45)cc3)c2)cc1 ; c1ccc(-c2ccc3ccc(-c4ccc(N(c5ccc(-c6cccc7sc8ccc9c%10ccccc%10oc9c8c67)cc5)c5cccc6c5sc5ccccc56)cc4)cc3c2)cc1]

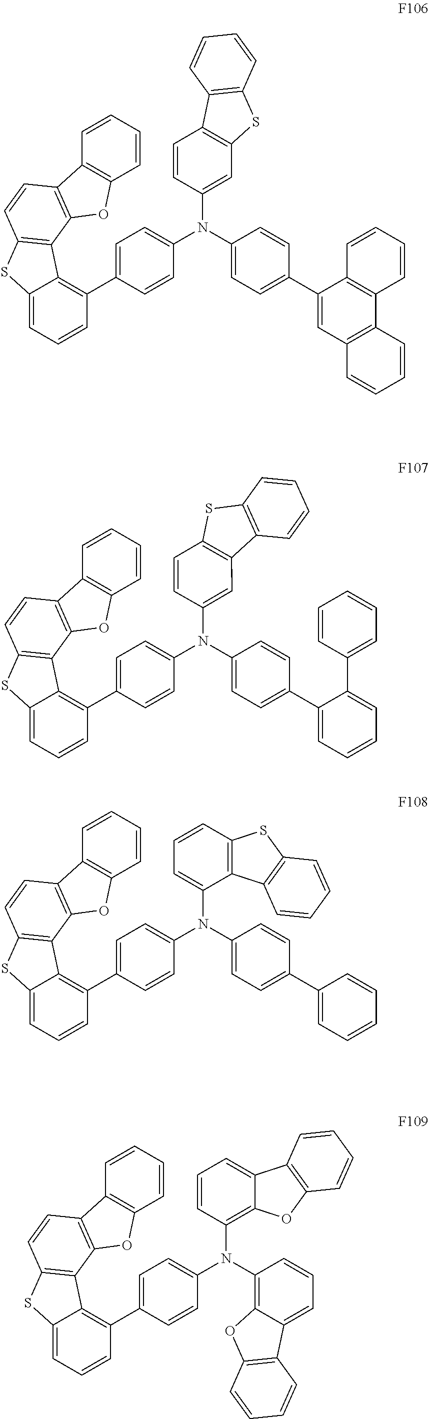 [SMILES: c1ccc2c(c1)cc(-c1ccc(N(c3ccc(-c4cccc5sc6ccc7c8ccccc8oc7c6c45)cc3)c3ccc4c(c3)sc3ccccc34)cc1)c1ccccc12 ; c1ccc(-c2ccccc2-c2ccc(N(c3ccc(-c4cccc5sc6ccc7c8ccccc8oc7c6c45)cc3)c3ccc4sc5ccccc5c4c3)cc2)cc1 ; c1ccc(-c2ccc(N(c3ccc(-c4cccc5sc6ccc7c8ccccc8oc7c6c45)cc3)c3cccc4sc5ccccc5c34)cc2)cc1 ; c1ccc2c(c1)oc1c(N(c3ccc(-c4cccc5sc6ccc7c8ccccc8oc7c6c45)cc3)c3cccc4c3oc3ccccc34)cccc12]

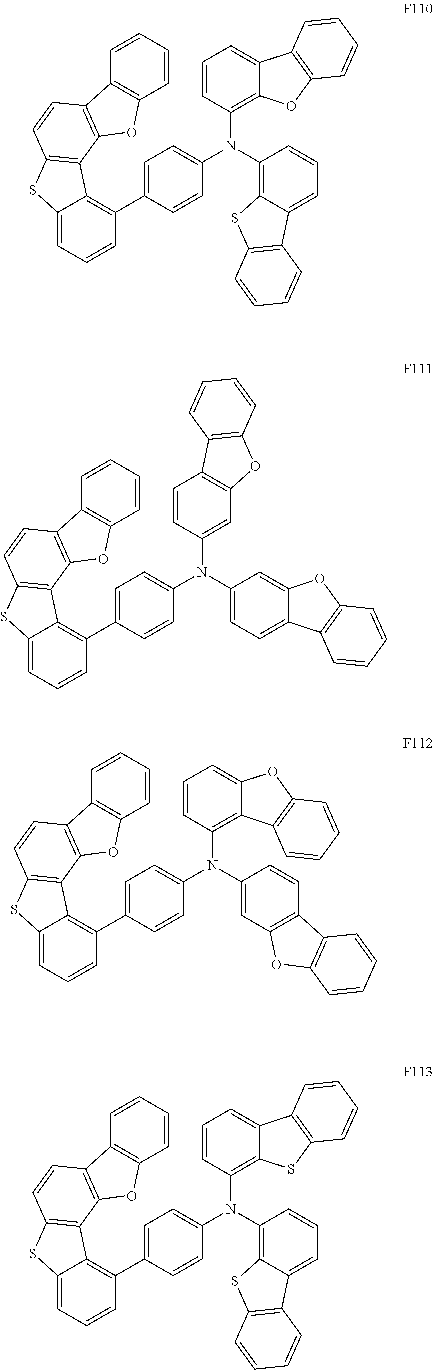 [SMILES: c1ccc2c(c1)oc1c(N(c3ccc(-c4cccc5sc6ccc7c8ccccc8oc7c6c45)cc3)c3cccc4c3sc3ccccc34)cccc12 ; c1ccc2c(c1)oc1cc(N(c3ccc(-c4cccc5sc6ccc7c8ccccc8oc7c6c45)cc3)c3ccc4c(c3)oc3ccccc34)ccc12 ; c1ccc2c(c1)oc1cc(N(c3ccc(-c4cccc5sc6ccc7c8ccccc8oc7c6c45)cc3)c3cccc4oc5ccccc5c34)ccc12 ; c1ccc2c(c1)oc1c2ccc2sc3cccc(-c4ccc(N(c5cccc6c5sc5ccccc56)c5cccc6c5sc5ccccc56)cc4)c3c21]

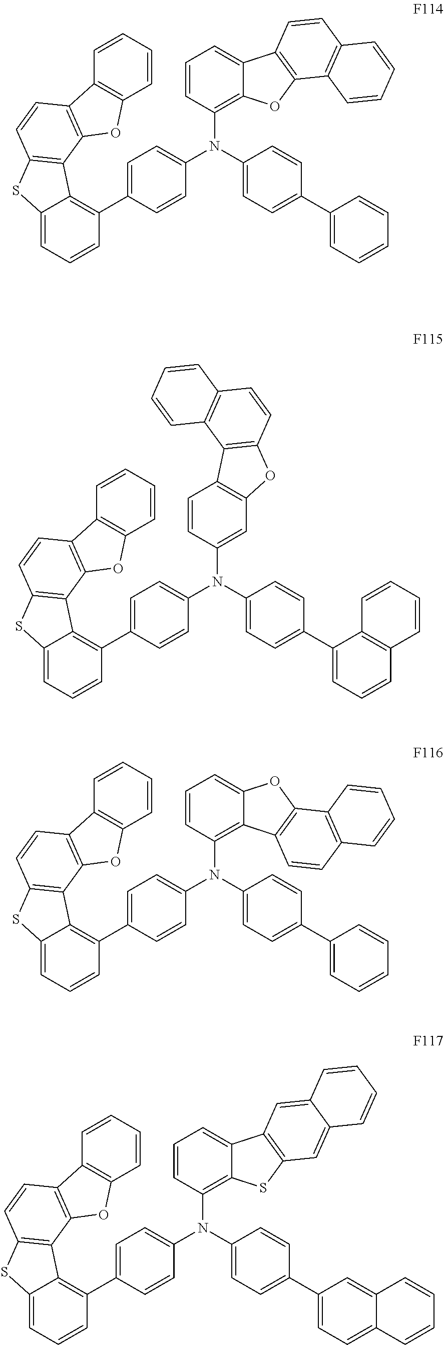 [SMILES: c1ccc(-c2ccc(N(c3ccc(-c4cccc5sc6ccc7c8ccccc8oc7c6c45)cc3)c3cccc4c3oc3c5ccccc5ccc43)cc2)cc1 ; c1ccc2c(-c3ccc(N(c4ccc(-c5cccc6sc7ccc8c9ccccc9oc8c7c56)cc4)c4ccc5c(c4)oc4ccc6ccccc6c45)cc3)cccc2c1 ; c1ccc(-c2ccc(N(c3ccc(-c4cccc5sc6ccc7c8ccccc8oc7c6c45)cc3)c3cccc4oc5c6ccccc6ccc5c34)cc2)cc1 ; c1ccc2cc(-c3ccc(N(c4ccc(-c5cccc6sc7ccc8c9ccccc9oc8c7c56)cc4)c4cccc5c4sc4cc6ccccc6cc45)cc3)ccc2c1]

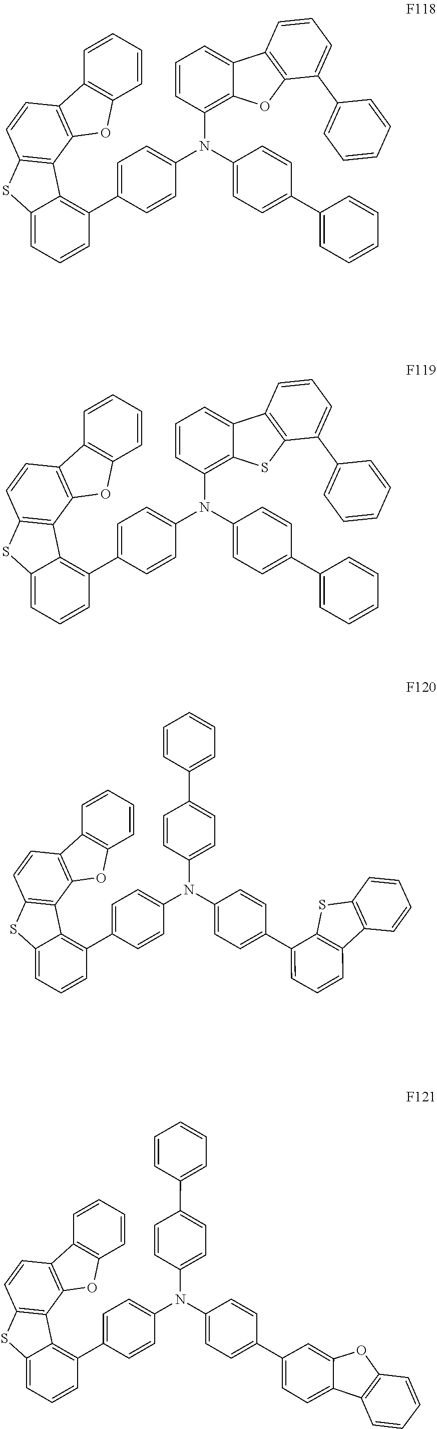 [SMILES: c1ccc(-c2ccc(N(c3ccc(-c4cccc5sc6ccc7c8ccccc8oc7c6c45)cc3)c3cccc4c3oc3c(-c5ccccc5)cccc34)cc2)cc1 ; c1ccc(-c2ccc(N(c3ccc(-c4cccc5sc6ccc7c8ccccc8oc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5sc6ccc7c8ccccc8oc7c6c45)cc3)cc2)cc1 ; c1ccc(-c2ccc(N(c3ccc(-c4ccc5c(c4)oc4ccccc45)cc3)c3ccc(-c4cccc5sc6ccc7c8ccccc8oc7c6c45)cc3)cc2)cc1]

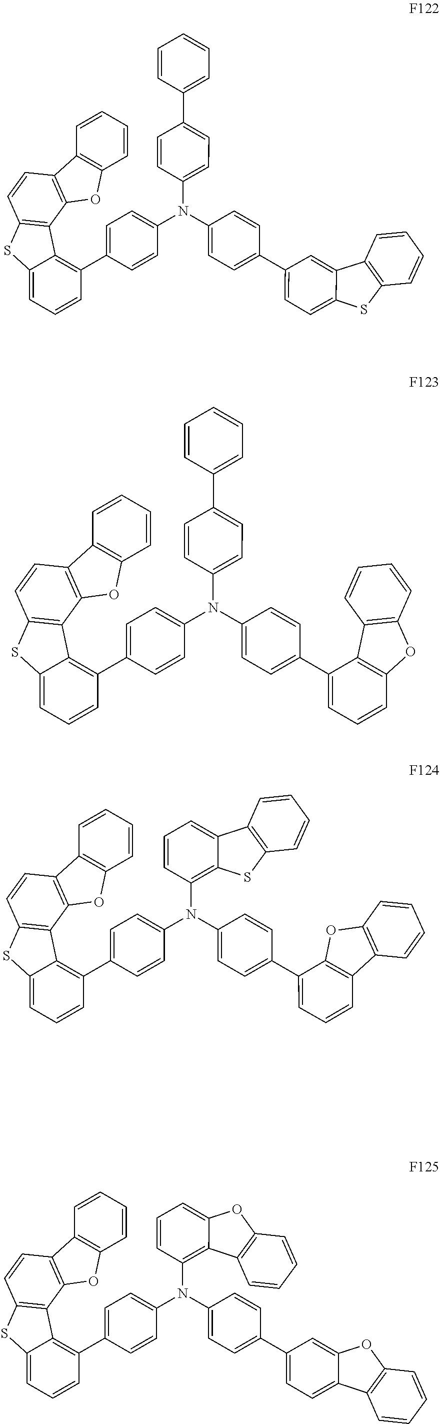 [SMILES: c1ccc(-c2ccc(N(c3ccc(-c4ccc5sc6ccccc6c5c4)cc3)c3ccc(-c4cccc5sc6ccc7c8ccccc8oc7c6c45)cc3)cc2)cc1 ; c1ccc(-c2ccc(N(c3ccc(-c4cccc5oc6ccccc6c45)cc3)c3ccc(-c4cccc5sc6ccc7c8ccccc8oc7c6c45)cc3)cc2)cc1 ; c1ccc2c(c1)oc1c(-c3ccc(N(c4ccc(-c5cccc6sc7ccc8c9ccccc9oc8c7c56)cc4)c4cccc5c4sc4ccccc45)cc3)cccc12 ; c1ccc2c(c1)oc1cc(-c3ccc(N(c4ccc(-c5cccc6sc7ccc8c9ccccc9oc8c7c56)cc4)c4cccc5oc6ccccc6c45)cc3)ccc12]

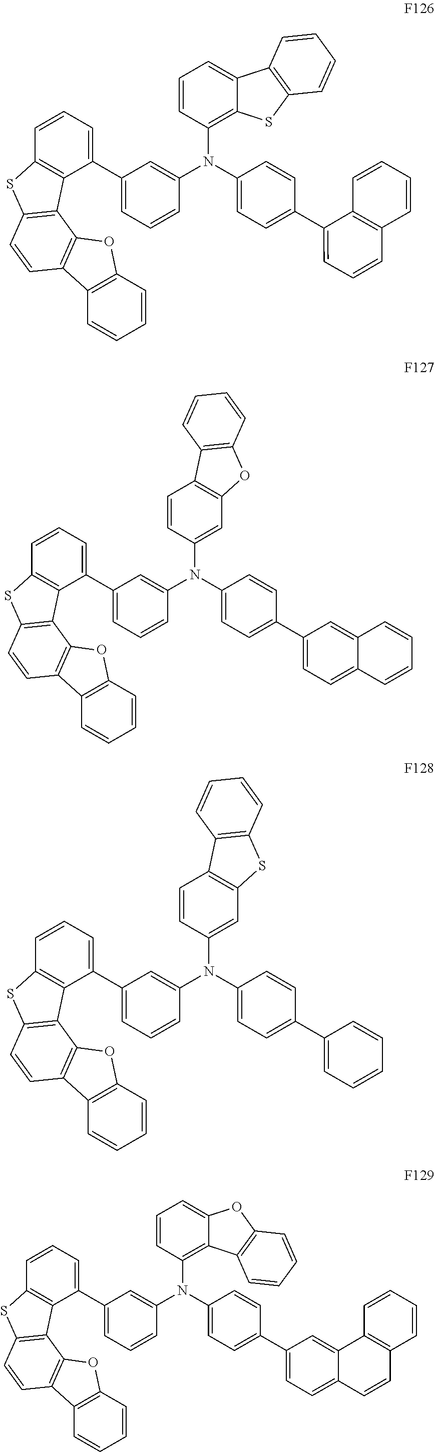 [SMILES: c1cc(-c2cccc3sc4ccc5c6ccccc6oc5c4c23)cc(N(c2ccc(-c3cccc4ccccc34)cc2)c2cccc3c2sc2ccccc23)c1 ; c1cc(-c2cccc3sc4ccc5c6ccccc6oc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5sc6ccc7c8ccccc8oc7c6c45)c3)c3ccc4c(c3)sc3ccccc34)cc2)cc1 ; c1cc(-c2cccc3sc4ccc5c6ccccc6oc5c4c23)cc(N(c2ccc(-c3ccc4ccc5ccccc5c4c3)cc2)c2cccc3oc4ccccc4c23)c1]

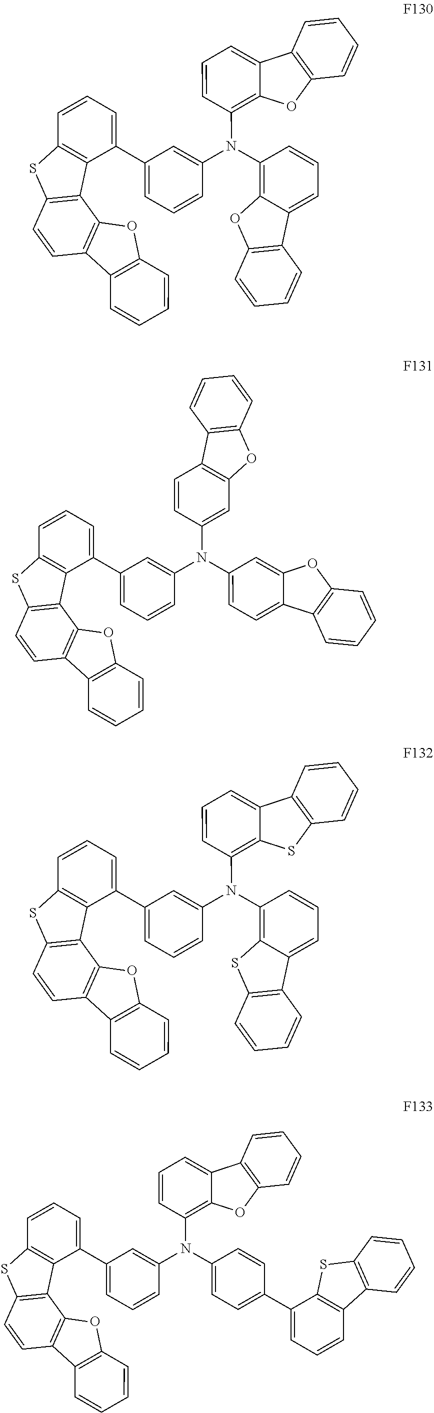 [SMILES: c1cc(-c2cccc3sc4ccc5c6ccccc6oc5c4c23)cc(N(c2cccc3c2oc2ccccc23)c2cccc3c2oc2ccccc23)c1 ; c1cc(-c2cccc3sc4ccc5c6ccccc6oc5c4c23)cc(N(c2ccc3c(c2)oc2ccccc23)c2ccc3c(c2)oc2ccccc23)c1 ; c1cc(-c2cccc3sc4ccc5c6ccccc6oc5c4c23)cc(N(c2cccc3c2sc2ccccc23)c2cccc3c2sc2ccccc23)c1 ; c1cc(-c2cccc3sc4ccc5c6ccccc6oc5c4c23)cc(N(c2ccc(-c3cccc4c3sc3ccccc34)cc2)c2cccc3c2oc2ccccc23)c1]

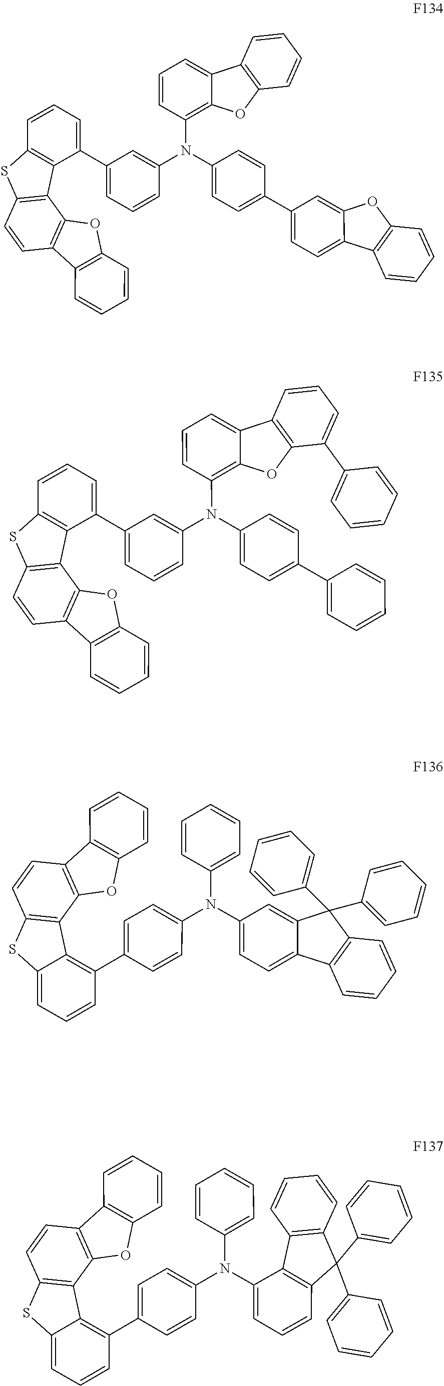 [SMILES: c1cc(-c2cccc3sc4ccc5c6ccccc6oc5c4c23)cc(N(c2ccc(-c3ccc4c(c3)oc3ccccc34)cc2)c2cccc3c2oc2ccccc23)c1 ; c1ccc(-c2ccc(N(c3cccc(-c4cccc5sc6ccc7c8ccccc8oc7c6c45)c3)c3cccc4c3oc3c(-c5ccccc5)cccc34)cc2)cc1 ; c1ccc(N(c2ccc(-c3cccc4sc5ccc6c7ccccc7oc6c5c34)cc2)c2ccc3c(c2)C(c2ccccc2)(c2ccccc2)c2ccccc2-3)cc1 ; c1ccc(N(c2ccc(-c3cccc4sc5ccc6c7ccccc7oc6c5c34)cc2)c2cccc3c2-c2ccccc2C3(c2ccccc2)c2ccccc2)cc1]

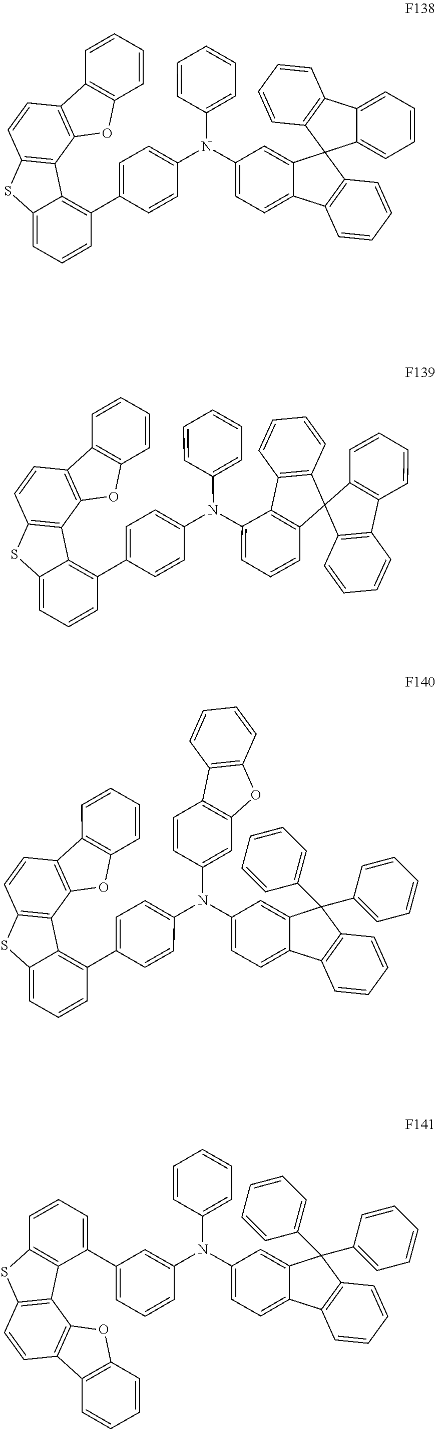 [SMILES: c1ccc(N(c2ccc(-c3cccc4sc5ccc6c7ccccc7oc6c5c34)cc2)c2ccc3c(c2)C2(c4ccccc4-c4ccccc42)c2ccccc2-3)cc1 ; c1ccc(N(c2ccc(-c3cccc4sc5ccc6c7ccccc7oc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6sc7ccc8c9ccccc9oc8c7c56)cc4)c4ccc5c(c4)oc4ccccc45)cc32)cc1 ; c1ccc(N(c2cccc(-c3cccc4sc5ccc6c7ccccc7oc6c5c34)c2)c2ccc3c(c2)C(c2ccccc2)(c2ccccc2)c2ccccc2-3)cc1]

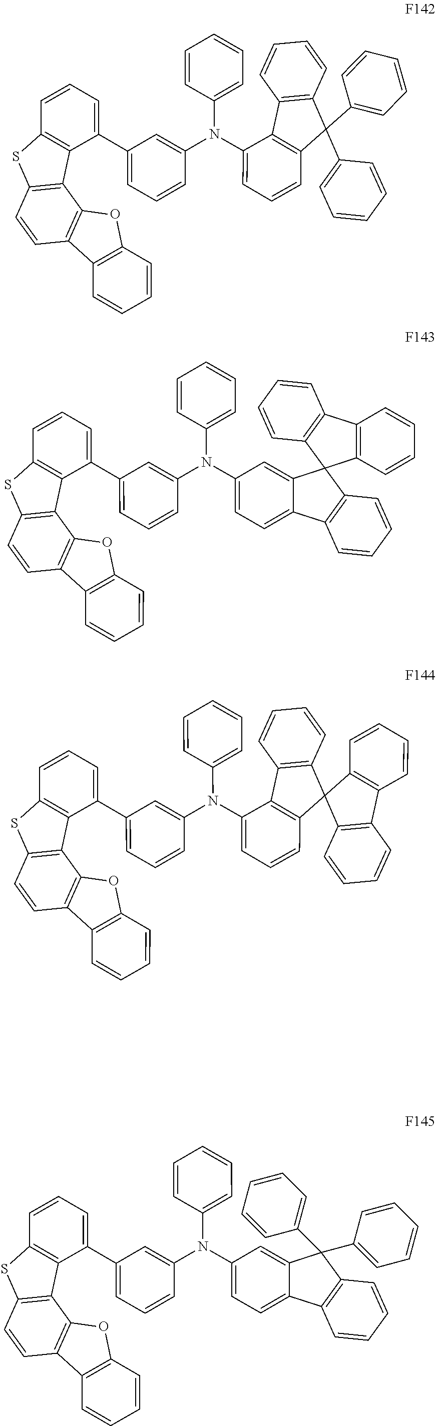 [SMILES: c1ccc(N(c2cccc(-c3cccc4sc5ccc6c7ccccc7oc6c5c34)c2)c2cccc3c2-c2ccccc2C3(c2ccccc2)c2ccccc2)cc1 ; c1ccc(N(c2cccc(-c3cccc4sc5ccc6c7ccccc7oc6c5c34)c2)c2ccc3c(c2)C2(c4ccccc4-c4ccccc42)c2ccccc2-3)cc1 ; c1ccc(N(c2cccc(-c3cccc4sc5ccc6c7ccccc7oc6c5c34)c2)c2cccc3c2-c2ccccc2C32c3ccccc3-c3ccccc32)cc1 ; c1ccc(N(c2cccc(-c3cccc4sc5ccc6c7ccccc7oc6c5c34)c2)c2ccc3c(c2)C(c2ccccc2)(c2ccccc2)c2ccccc2-3)cc1]

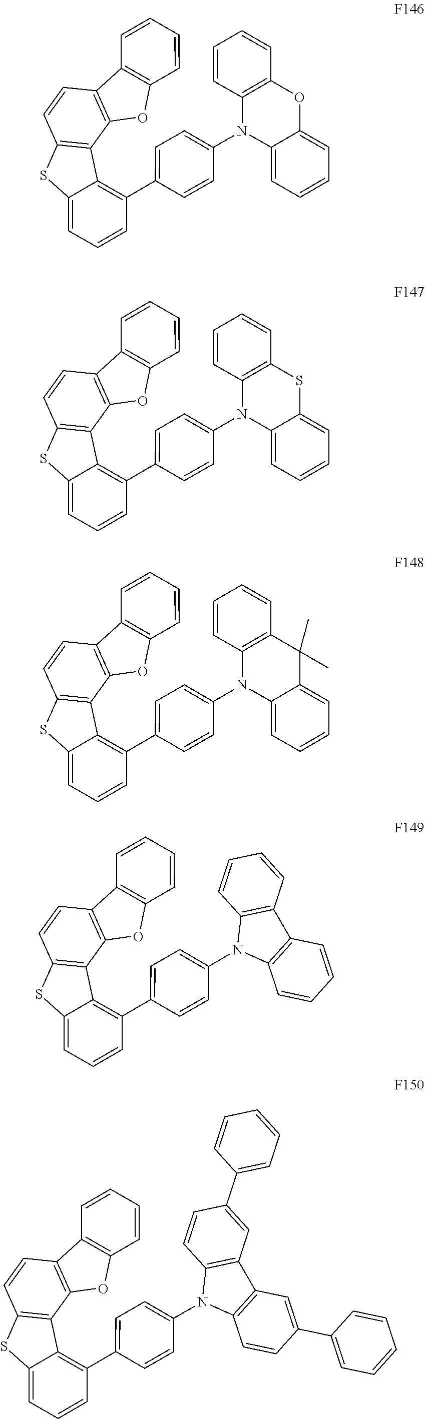 [SMILES: c1ccc2c(c1)Oc1ccccc1N2c1ccc(-c2cccc3sc4ccc5c6ccccc6oc5c4c23)cc1 ; c1ccc2c(c1)Sc1ccccc1N2c1ccc(-c2cccc3sc4ccc5c6ccccc6oc5c4c23)cc1 ; CC1(C)c2ccccc2N(c2ccc(-c3cccc4sc5ccc6c7ccccc7oc6c5c34)cc2)c2ccccc21 ; c1ccc2c(c1)oc1c2ccc2sc3cccc(-c4ccc(-n5c6ccccc6c6ccccc65)cc4)c3c21 ; c1ccc(-c2ccc3c(c2)c2cc(-c4ccccc4)ccc2n3-c2ccc(-c3cccc4sc5ccc6c7ccccc7oc6c5c34)cc2)cc1]

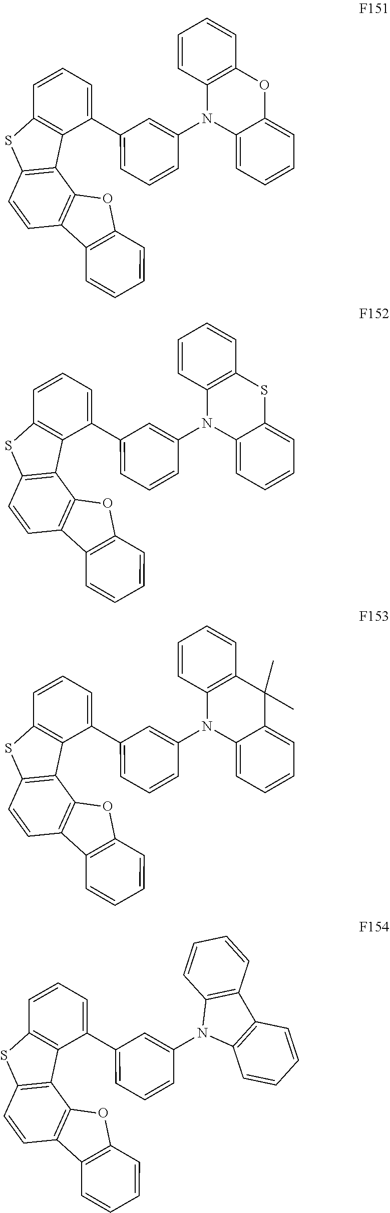 [SMILES: c1cc(-c2cccc3sc4ccc5c6ccccc6oc5c4c23)cc(N2c3ccccc3Oc3ccccc32)c1 ; c1cc(-c2cccc3sc4ccc5c6ccccc6oc5c4c23)cc(N2c3ccccc3Sc3ccccc32)c1 ; CC1(C)c2ccccc2N(c2cccc(-c3cccc4sc5ccc6c7ccccc7oc6c5c34)c2)c2ccccc21 ; c1cc(-c2cccc3sc4ccc5c6ccccc6oc5c4c23)cc(-n2c3ccccc3c3ccccc32)c1]

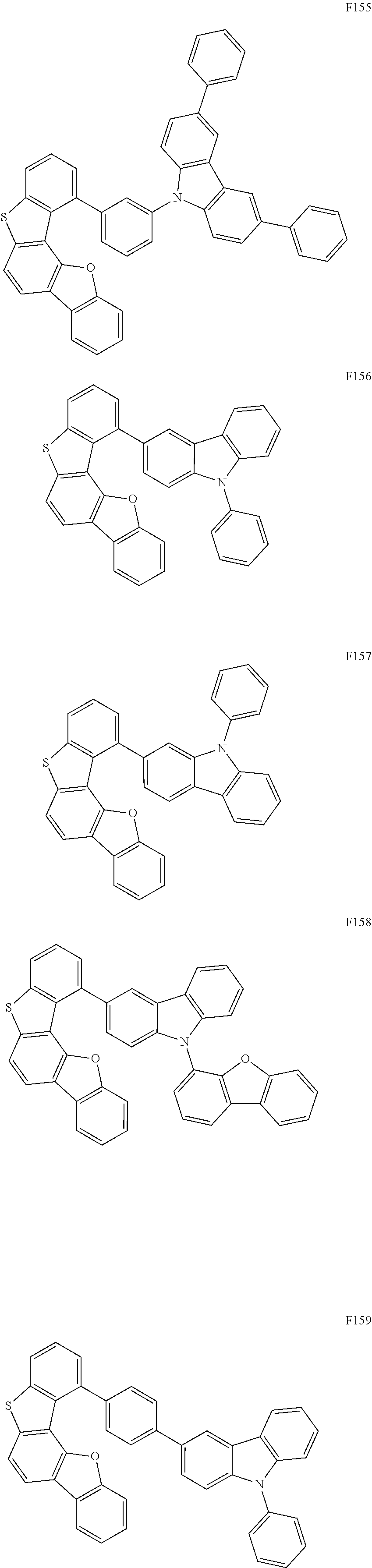

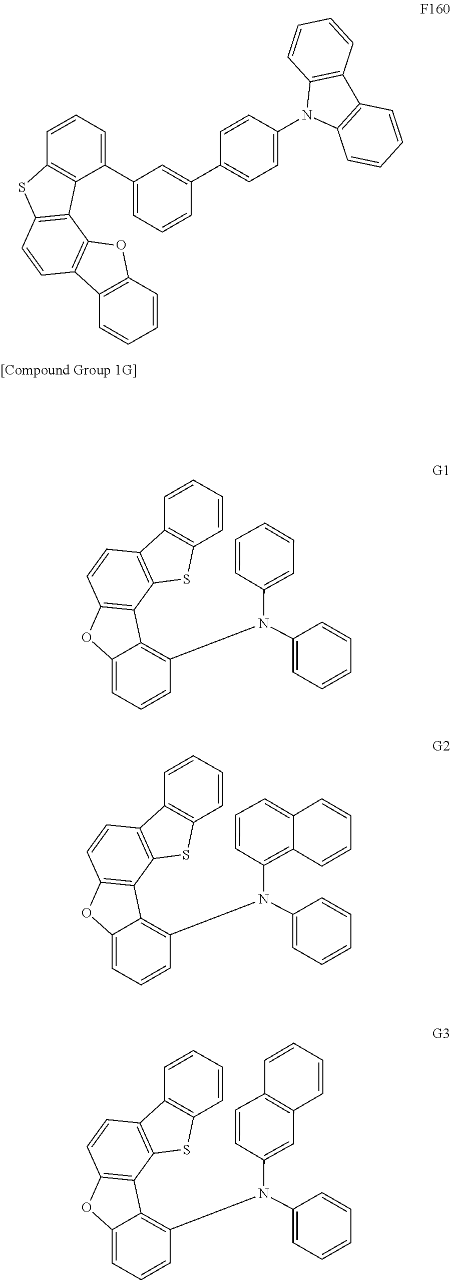 [SMILES: c1cc(-c2ccc(-n3c4ccccc4c4ccccc43)cc2)cc(-c2cccc3sc4ccc5c6ccccc6oc5c4c23)c1 ; c1ccc(N(c2ccccc2)c2cccc3oc4ccc5c6ccccc6sc5c4c23)cc1 ; c1ccc(N(c2cccc3ccccc23)c2cccc3oc4ccc5c6ccccc6sc5c4c23)cc1 ; c1ccc(N(c2ccc3ccccc3c2)c2cccc3oc4ccc5c6ccccc6sc5c4c23)cc1]

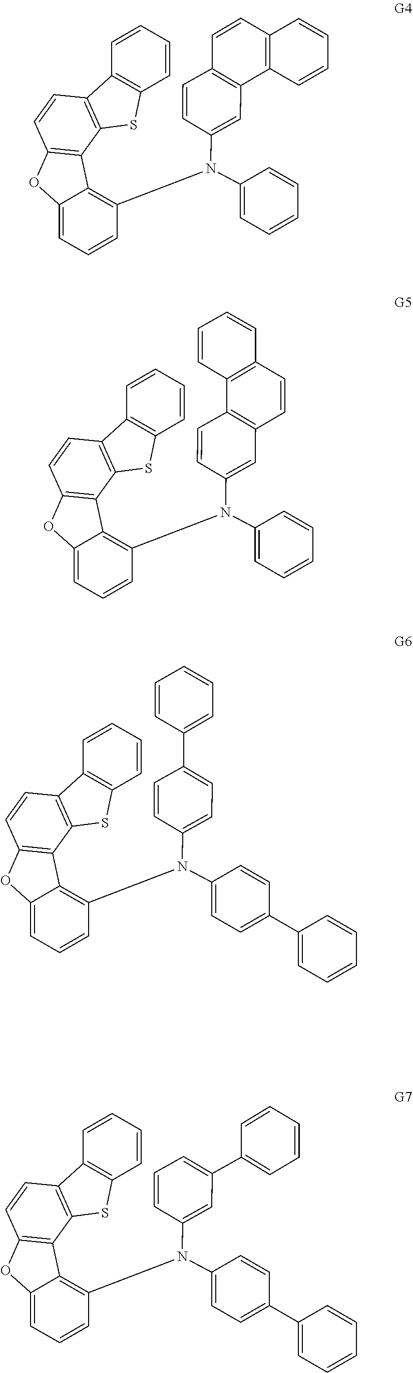 [SMILES: c1ccc(N(c2ccc3ccc4ccccc4c3c2)c2cccc3oc4ccc5c6ccccc6sc5c4c23)cc1 ; c1ccc(N(c2ccc3c(ccc4ccccc43)c2)c2cccc3oc4ccc5c6ccccc6sc5c4c23)cc1 ; c1ccc(-c2ccc(N(c3ccc(-c4ccccc4)cc3)c3cccc4oc5ccc6c7ccccc7sc6c5c34)cc2)cc1 ; c1ccc(-c2ccc(N(c3cccc(-c4ccccc4)c3)c3cccc4oc5ccc6c7ccccc7sc6c5c34)cc2)cc1]

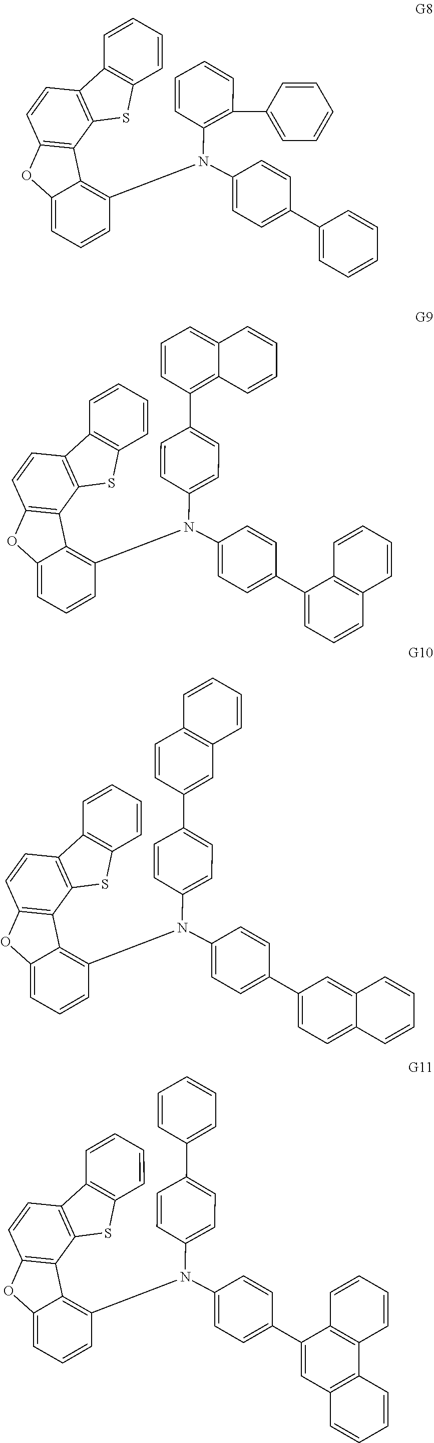 [SMILES: c1ccc(-c2ccc(N(c3ccccc3-c3ccccc3)c3cccc4oc5ccc6c7ccccc7sc6c5c34)cc2)cc1 ; c1ccc2c(-c3ccc(N(c4ccc(-c5cccc6ccccc56)cc4)c4cccc5oc6ccc7c8ccccc8sc7c6c45)cc3)cccc2c1 ; c1ccc2cc(-c3ccc(N(c4ccc(-c5ccc6ccccc6c5)cc4)c4cccc5oc6ccc7c8ccccc8sc7c6c45)cc3)ccc2c1 ; c1ccc(-c2ccc(N(c3ccc(-c4cc5ccccc5c5ccccc45)cc3)c3cccc4oc5ccc6c7ccccc7sc6c5c34)cc2)cc1]

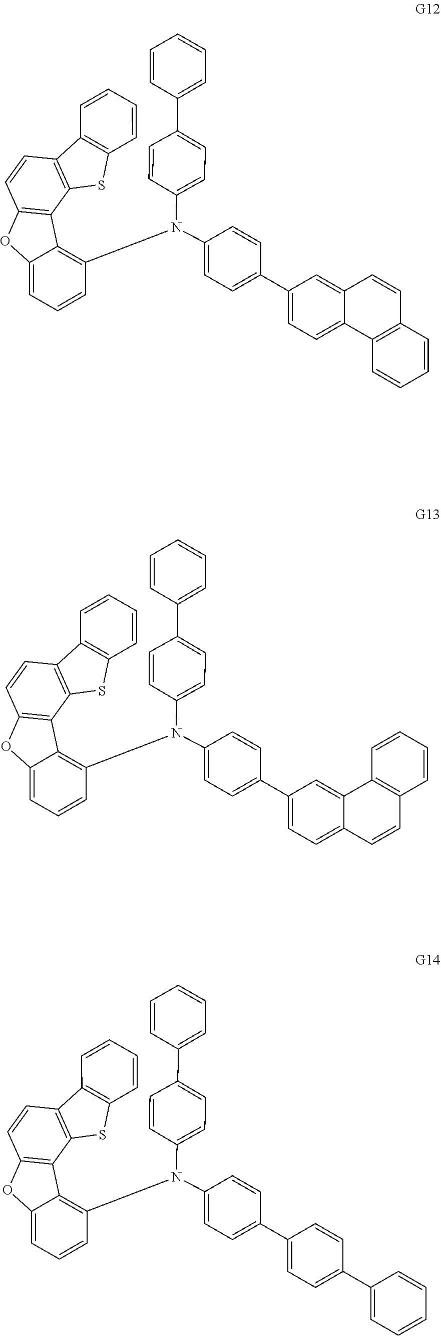 [SMILES: c1ccc(-c2ccc(N(c3ccc(-c4ccc5c(ccc6ccccc65)c4)cc3)c3cccc4oc5ccc6c7ccccc7sc6c5c34)cc2)cc1 ; c1ccc(-c2ccc(N(c3ccc(-c4ccc5ccc6ccccc6c5c4)cc3)c3cccc4oc5ccc6c7ccccc7sc6c5c34)cc2)cc1 ; c1ccc(-c2ccc(-c3ccc(N(c4ccc(-c5ccccc5)cc4)c4cccc5oc6ccc7c8ccccc8sc7c6c45)cc3)cc2)cc1]

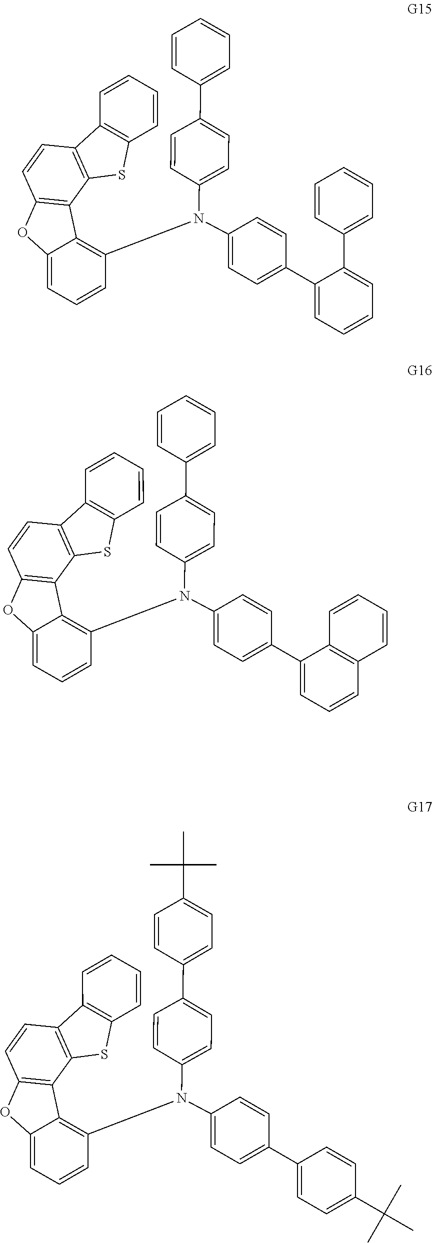 [SMILES: c1ccc(-c2ccc(N(c3ccc(-c4ccccc4-c4ccccc4)cc3)c3cccc4oc5ccc6c7ccccc7sc6c5c34)cc2)cc1 ; c1ccc(-c2ccc(N(c3ccc(-c4cccc5ccccc45)cc3)c3cccc4oc5ccc6c7ccccc7sc6c5c34)cc2)cc1 ; CC(C)(C)c1ccc(-c2ccc(N(c3ccc(-c4ccc(C(C)(C)C)cc4)cc3)c3cccc4oc5ccc6c7ccccc7sc6c5c34)cc2)cc1]

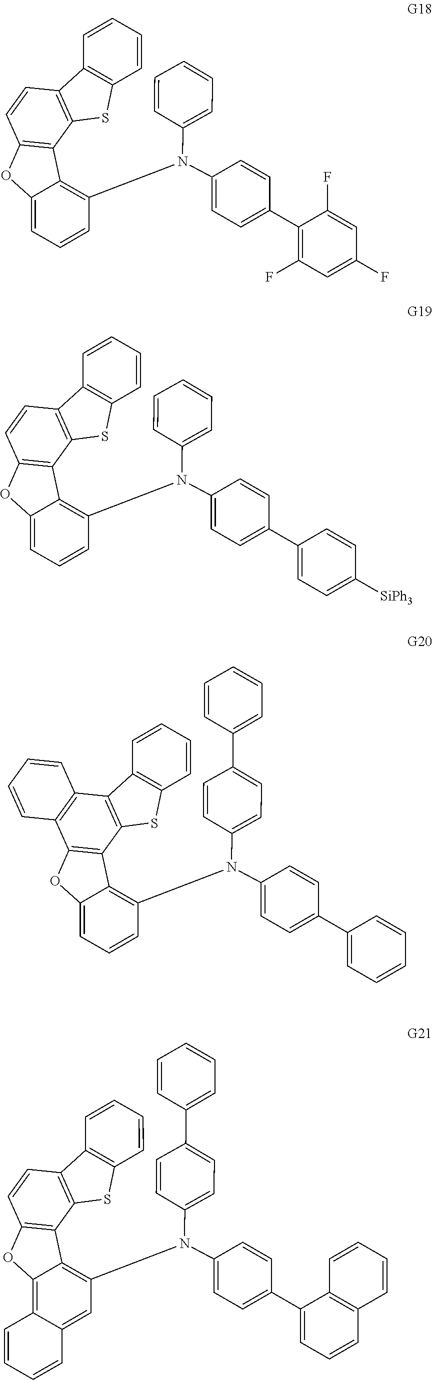

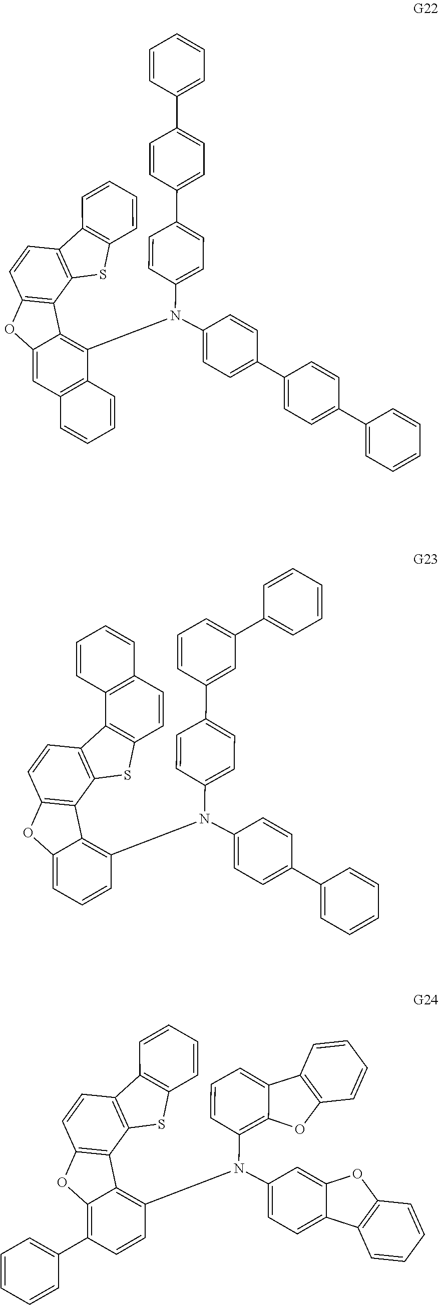 [SMILES: c1ccc(-c2ccc(-c3ccc(N(c4ccc(-c5ccc(-c6ccccc6)cc5)cc4)c4c5ccccc5cc5oc6ccc7c8ccccc8sc7c6c45)cc3)cc2)cc1 ; c1ccc(-c2ccc(N(c3ccc(-c4cccc(-c5ccccc5)c4)cc3)c3cccc4oc5ccc6c(sc7ccc8ccccc8c76)c5c34)cc2)cc1 ; c1ccc(-c2ccc(N(c3ccc4c(c3)oc3ccccc34)c3cccc4c3oc3ccccc34)c3c2oc2ccc4c5ccccc5sc4c23)cc1]

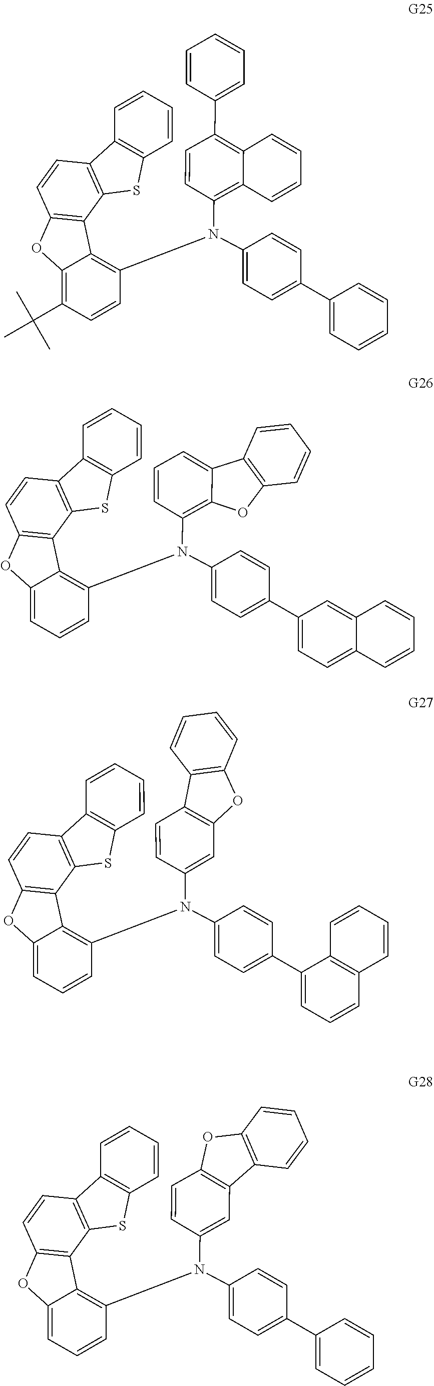 [SMILES: CC(C)(C)c1ccc(N(c2ccc(-c3ccccc3)cc2)c2ccc(-c3ccccc3)c3ccccc23)c2c1oc1ccc3c4ccccc4sc3c12 ; c1ccc2cc(-c3ccc(N(c4cccc5c4oc4ccccc45)c4cccc5oc6ccc7c8ccccc8sc7c6c45)cc3)ccc2c1 ; c1ccc2c(-c3ccc(N(c4ccc5c(c4)oc4ccccc45)c4cccc5oc6ccc7c8ccccc8sc7c6c45)cc3)cccc2c1 ; c1ccc(-c2ccc(N(c3ccc4oc5ccccc5c4c3)c3cccc4oc5ccc6c7ccccc7sc6c5c34)cc2)cc1]

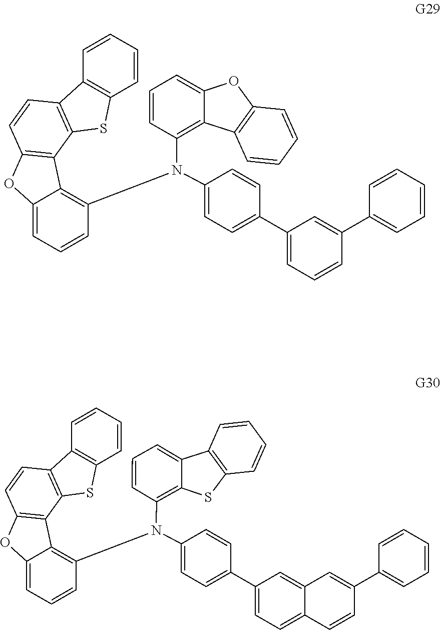 [SMILES: c1ccc(-c2cccc(-c3ccc(N(c4cccc5oc6ccccc6c45)c4cccc5oc6ccc7c8ccccc8sc7c6c45)cc3)c2)cc1 ; c1ccc(-c2ccc3ccc(-c4ccc(N(c5cccc6c5sc5ccccc56)c5cccc6oc7ccc8c9ccccc9sc8c7c56)cc4)cc3c2)cc1]

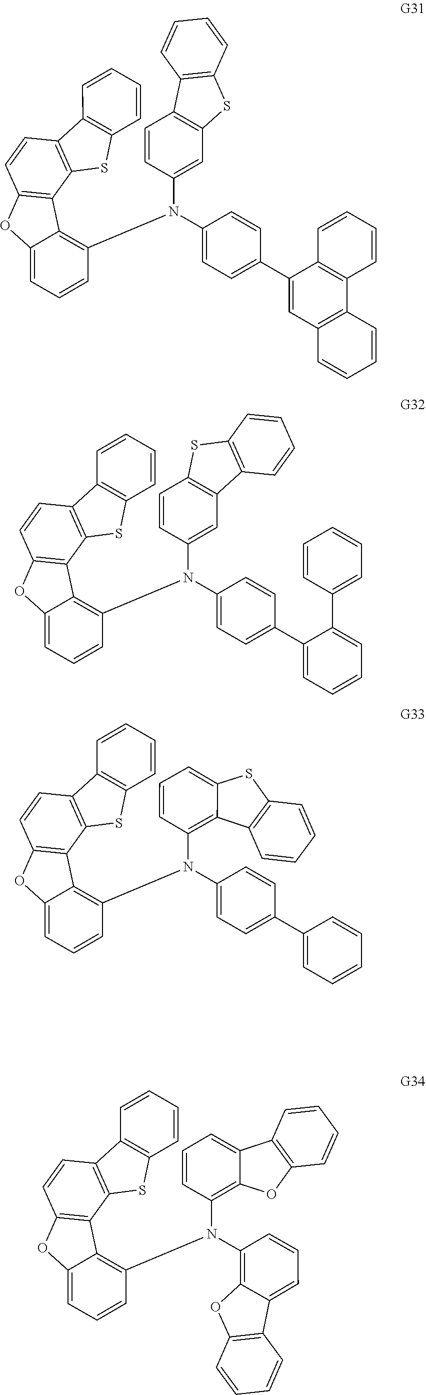 [SMILES: c1ccc2c(c1)cc(-c1ccc(N(c3ccc4c(c3)sc3ccccc34)c3cccc4oc5ccc6c7ccccc7sc6c5c34)cc1)c1ccccc12 ; c1ccc(-c2ccccc2-c2ccc(N(c3ccc4sc5ccccc5c4c3)c3cccc4oc5ccc6c7ccccc7sc6c5c34)cc2)cc1 ; c1ccc(-c2ccc(N(c3cccc4sc5ccccc5c34)c3cccc4oc5ccc6c7ccccc7sc6c5c34)cc2)cc1 ; c1ccc2c(c1)oc1c(N(c3cccc4c3oc3ccccc34)c3cccc4oc5ccc6c7ccccc7sc6c5c34)cccc12]

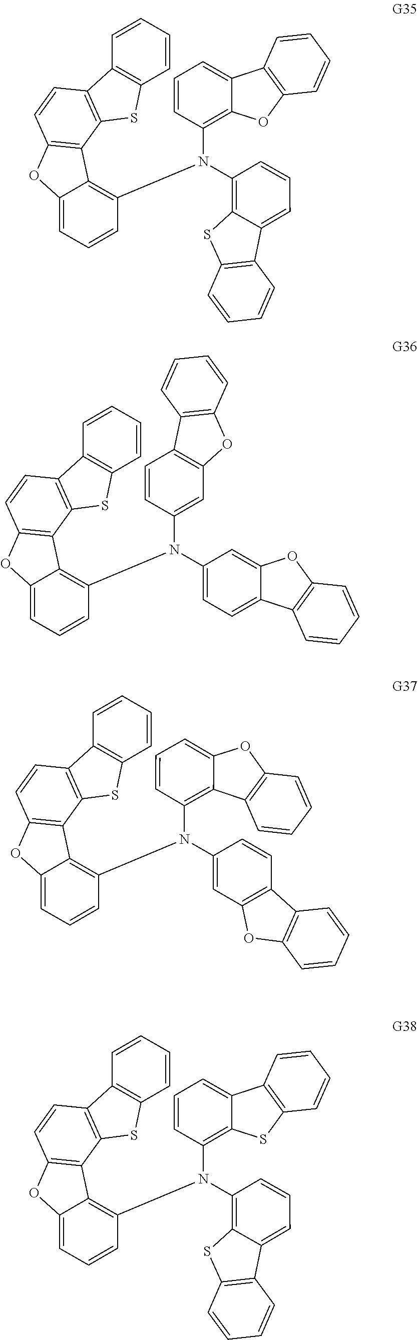

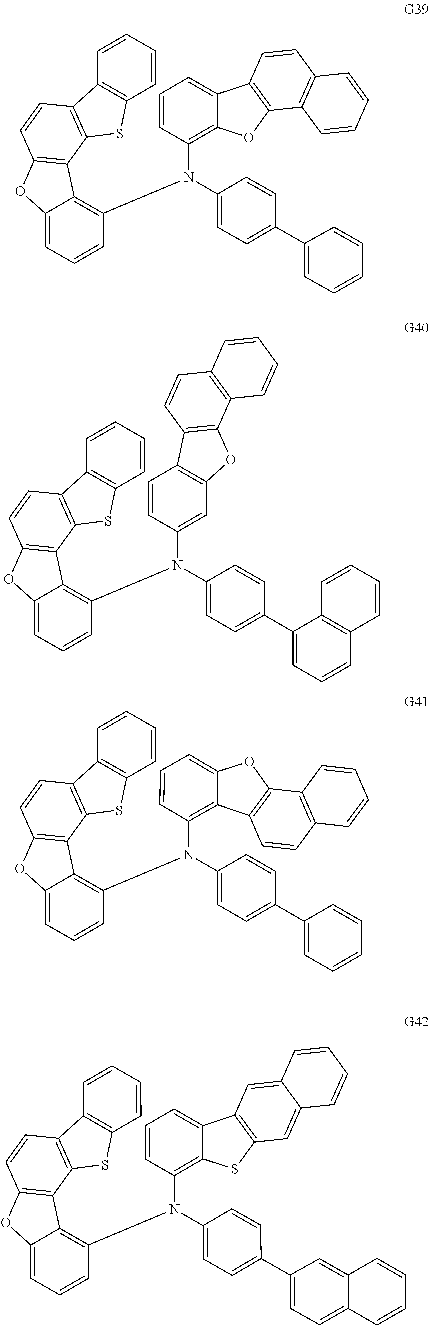 [SMILES: c1ccc(-c2ccc(N(c3cccc4c3oc3c5ccccc5ccc43)c3cccc4oc5ccc6c7ccccc7sc6c5c34)cc2)cc1 ; c1ccc2c(-c3ccc(N(c4ccc5c(c4)oc4c6ccccc6ccc54)c4cccc5oc6ccc7c8ccccc8sc7c6c45)cc3)cccc2c1 ; c1ccc(-c2ccc(N(c3cccc4oc5c6ccccc6ccc5c34)c3cccc4oc5ccc6c7ccccc7sc6c5c34)cc2)cc1 ; c1ccc2cc(-c3ccc(N(c4cccc5c4sc4cc6ccccc6cc45)c4cccc5oc6ccc7c8ccccc8sc7c6c45)cc3)ccc2c1]

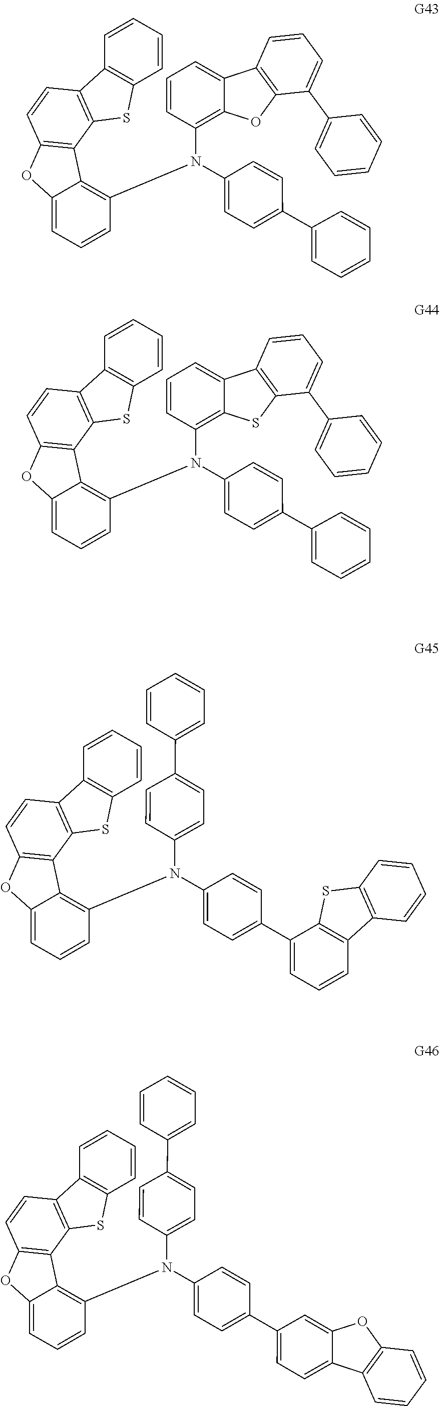 [SMILES: c1ccc(-c2ccc(N(c3cccc4c3oc3c(-c5ccccc5)cccc34)c3cccc4oc5ccc6c7ccccc7sc6c5c34)cc2)cc1 ; c1ccc(-c2ccc(N(c3cccc4c3sc3c(-c5ccccc5)cccc34)c3cccc4oc5ccc6c7ccccc7sc6c5c34)cc2)cc1 ; c1ccc(-c2ccc(N(c3ccc(-c4cccc5c4sc4ccccc45)cc3)c3cccc4oc5ccc6c7ccccc7sc6c5c34)cc2)cc1 ; c1ccc(-c2ccc(N(c3ccc(-c4ccc5c(c4)oc4ccccc45)cc3)c3cccc4oc5ccc6c7ccccc7sc6c5c34)cc2)cc1]

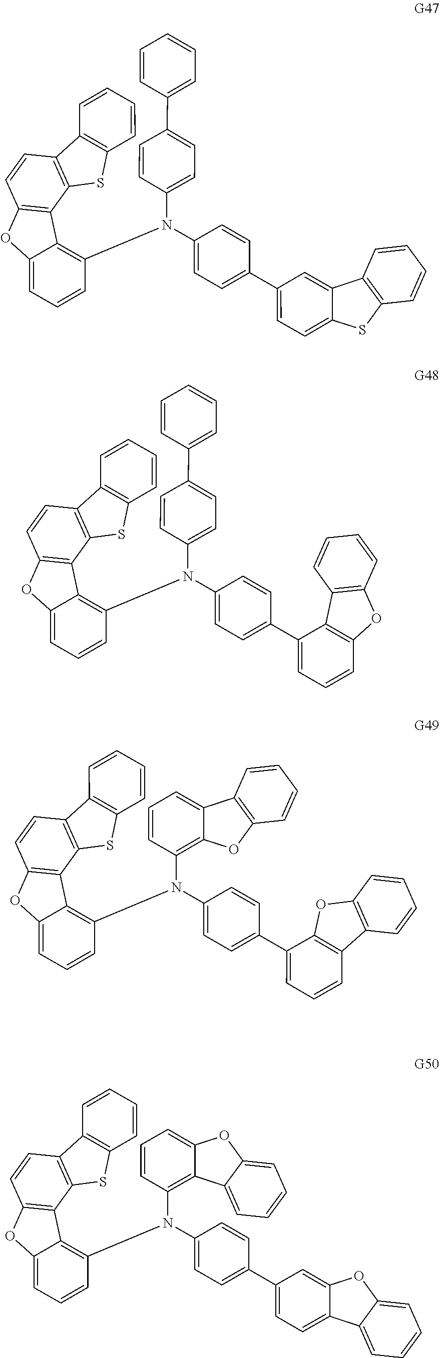 [SMILES: c1ccc(-c2ccc(N(c3ccc(-c4ccc5sc6ccccc6c5c4)cc3)c3cccc4oc5ccc6c7ccccc7sc6c5c34)cc2)cc1 ; c1ccc(-c2ccc(N(c3ccc(-c4cccc5oc6ccccc6c45)cc3)c3cccc4oc5ccc6c7ccccc7sc6c5c34)cc2)cc1 ; c1ccc2c(c1)oc1c(-c3ccc(N(c4cccc5c4oc4ccccc45)c4cccc5oc6ccc7c8ccccc8sc7c6c45)cc3)cccc12 ; c1ccc2c(c1)oc1cc(-c3ccc(N(c4cccc5oc6ccccc6c45)c4cccc5oc6ccc7c8ccccc8sc7c6c45)cc3)ccc12]

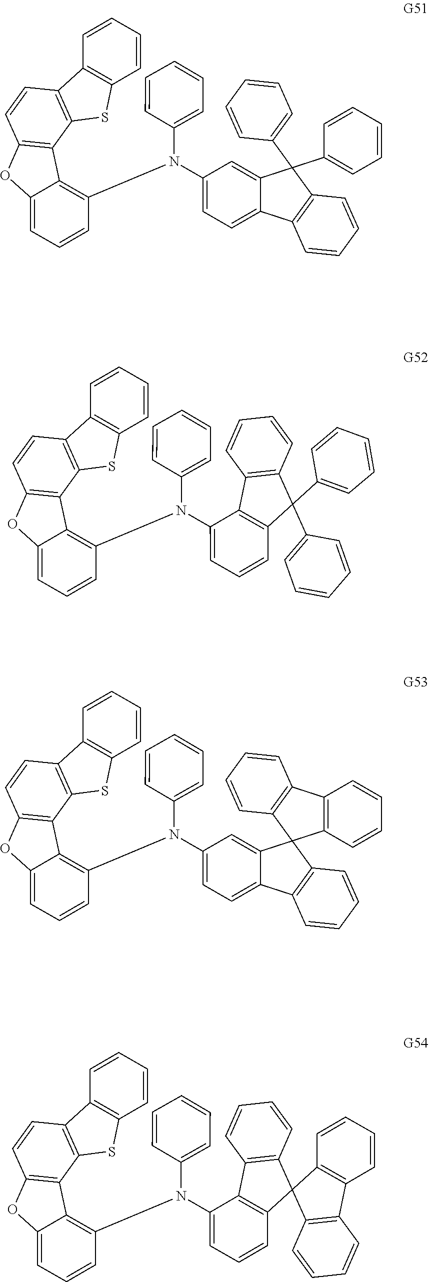 [SMILES: c1ccc(N(c2ccc3c(c2)C(c2ccccc2)(c2ccccc2)c2ccccc2-3)c2cccc3oc4ccc5c6ccccc6sc5c4c23)cc1 ; c1ccc(N(c2cccc3c2-c2ccccc2C3(c2ccccc2)c2ccccc2)c2cccc3oc4ccc5c6ccccc6sc5c4c23)cc1 ; c1ccc(N(c2ccc3c(c2)C2(c4ccccc4-c4ccccc42)c2ccccc2-3)c2cccc3oc4ccc5c6ccccc6sc5c4c23)cc1 ; c1ccc(N(c2cccc3c2-c2ccccc2C32c3ccccc3-c3ccccc32)c2cccc3oc4ccc5c6ccccc6sc5c4c23)cc1]

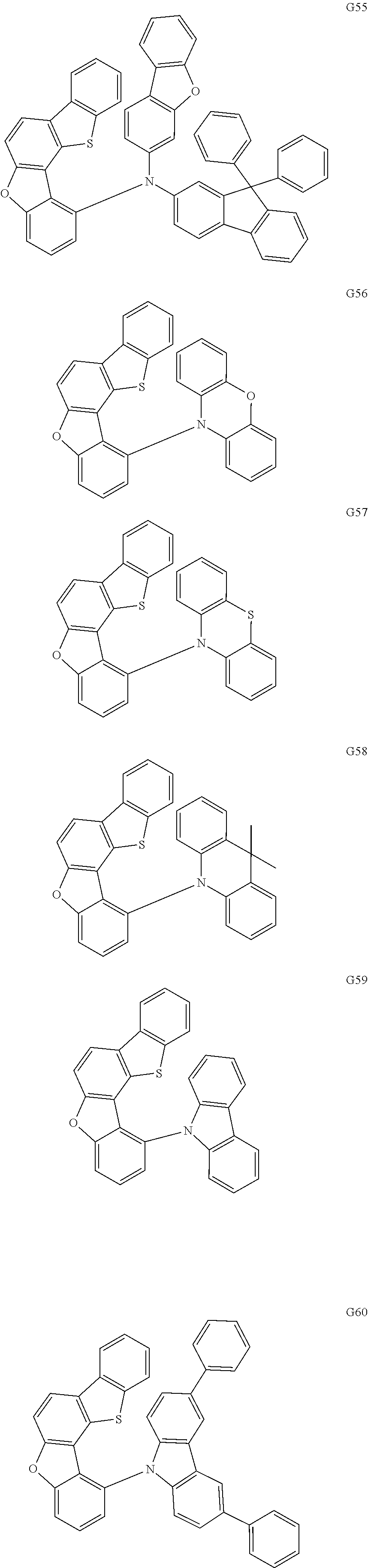 [SMILES: c1ccc(C2(c3ccccc3)c3ccccc3-c3ccc(N(c4ccc5c(c4)oc4ccccc45)c4cccc5oc6ccc7c8ccccc8sc7c6c45)cc32)cc1 ; c1ccc2c(c1)Oc1ccccc1N2c1cccc2oc3ccc4c5ccccc5sc4c3c12 ; c1ccc2c(c1)Sc1ccccc1N2c1cccc2oc3ccc4c5ccccc5sc4c3c12 ; CC1(C)c2ccccc2N(c2cccc3oc4ccc5c6ccccc6sc5c4c23)c2ccccc21 ; c1cc(-n2c3ccccc3c3ccccc32)c2c(c1)oc1ccc3c4ccccc4sc3c12 ; c1ccc(-c2ccc3c(c2)c2cc(-c4ccccc4)ccc2n3-c2cccc3oc4ccc5c6ccccc6sc5c4c23)cc1]

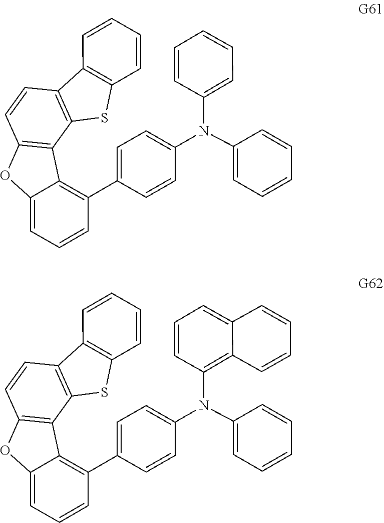 [SMILES: c1ccc(N(c2ccccc2)c2ccc(-c3cccc4oc5ccc6c7ccccc7sc6c5c34)cc2)cc1 ; c1ccc(N(c2ccc(-c3cccc4oc5ccc6c7ccccc7sc6c5c34)cc2)c2cccc3ccccc23)cc1]

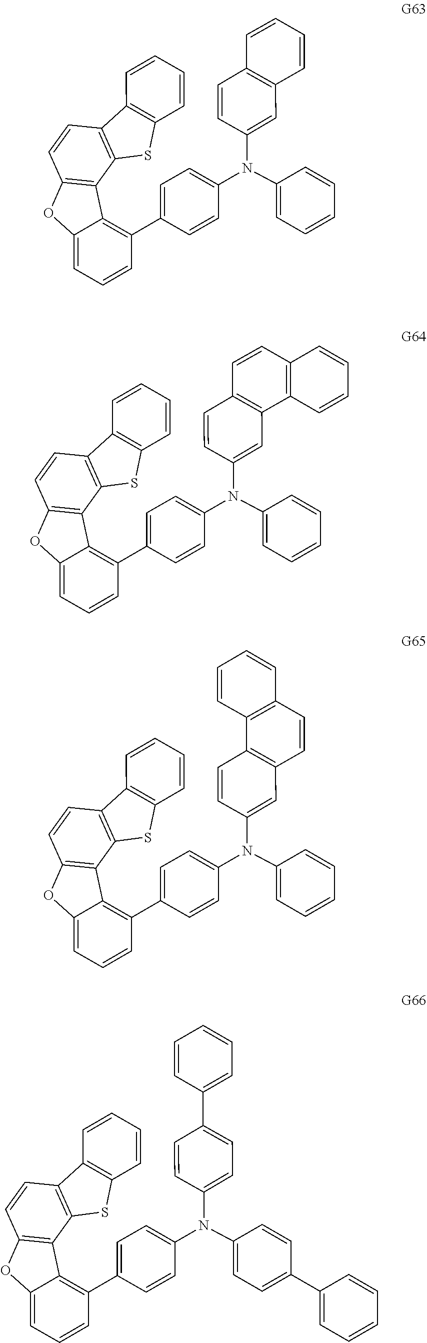 [SMILES: c1ccc(N(c2ccc(-c3cccc4oc5ccc6c7ccccc7sc6c5c34)cc2)c2ccc3ccccc3c2)cc1 ; c1ccc(N(c2ccc(-c3cccc4oc5ccc6c7ccccc7sc6c5c34)cc2)c2ccc3ccc4ccccc4c3c2)cc1 ; c1ccc(N(c2ccc(-c3cccc4oc5ccc6c7ccccc7sc6c5c34)cc2)c2ccc3c(ccc4ccccc43)c2)cc1 ; c1ccc(-c2ccc(N(c3ccc(-c4ccccc4)cc3)c3ccc(-c4cccc5oc6ccc7c8ccccc8sc7c6c45)cc3)cc2)cc1]

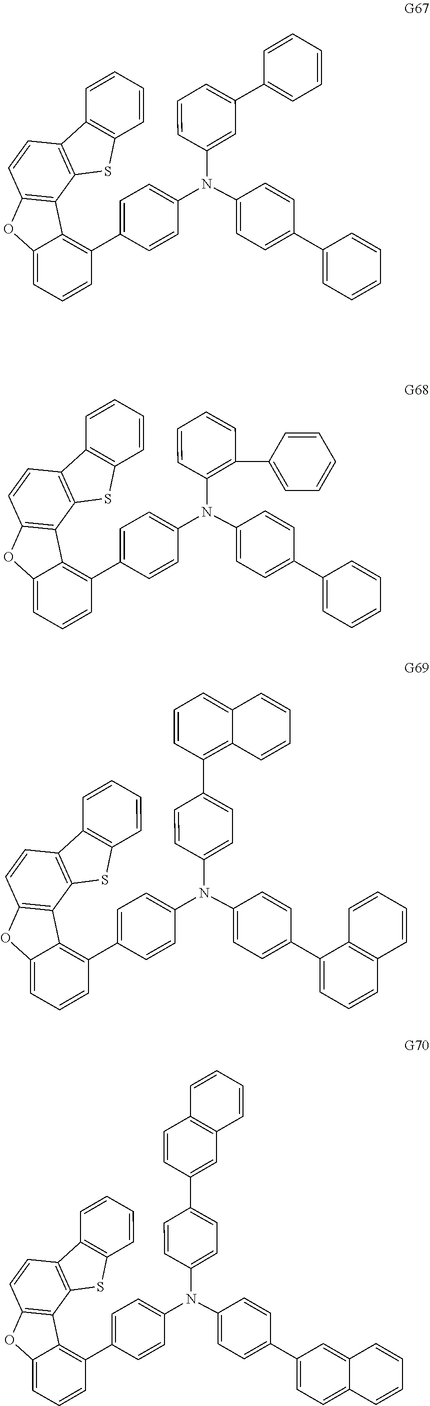 [SMILES: c1ccc(-c2ccc(N(c3ccc(-c4cccc5oc6ccc7c8ccccc8sc7c6c45)cc3)c3cccc(-c4ccccc4)c3)cc2)cc1 ; c1ccc(-c2ccc(N(c3ccc(-c4cccc5oc6ccc7c8ccccc8sc7c6c45)cc3)c3ccccc3-c3ccccc3)cc2)cc1 ; c1ccc2c(-c3ccc(N(c4ccc(-c5cccc6ccccc56)cc4)c4ccc(-c5cccc6oc7ccc8c9ccccc9sc8c7c56)cc4)cc3)cccc2c1 ; c1ccc2cc(-c3ccc(N(c4ccc(-c5ccc6ccccc6c5)cc4)c4ccc(-c5cccc6oc7ccc8c9ccccc9sc8c7c56)cc4)cc3)ccc2c1]

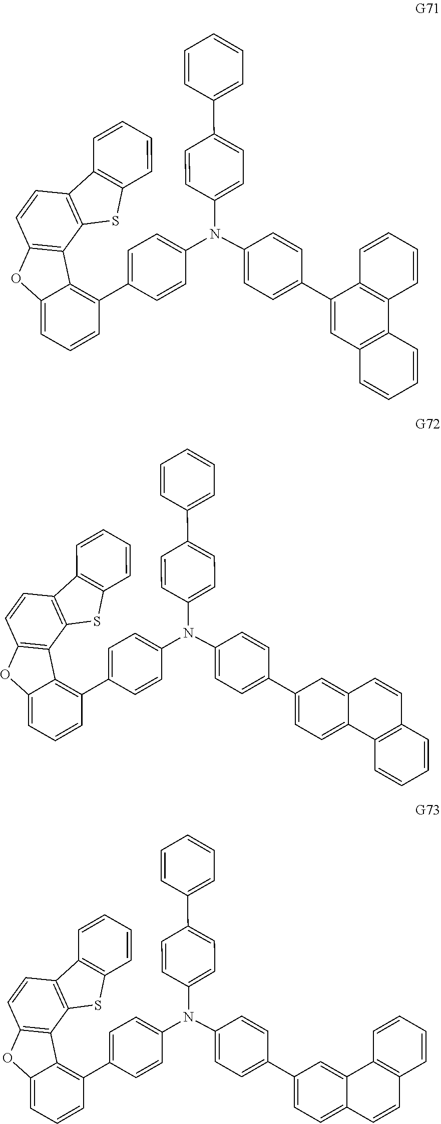 [SMILES: c1ccc(-c2ccc(N(c3ccc(-c4cc5ccccc5c5ccccc45)cc3)c3ccc(-c4cccc5oc6ccc7c8ccccc8sc7c6c45)cc3)cc2)cc1 ; c1ccc(-c2ccc(N(c3ccc(-c4ccc5c(ccc6ccccc65)c4)cc3)c3ccc(-c4cccc5oc6ccc7c8ccccc8sc7c6c45)cc3)cc2)cc1 ; c1ccc(-c2ccc(N(c3ccc(-c4ccc5ccc6ccccc6c5c4)cc3)c3ccc(-c4cccc5oc6ccc7c8ccccc8sc7c6c45)cc3)cc2)cc1]

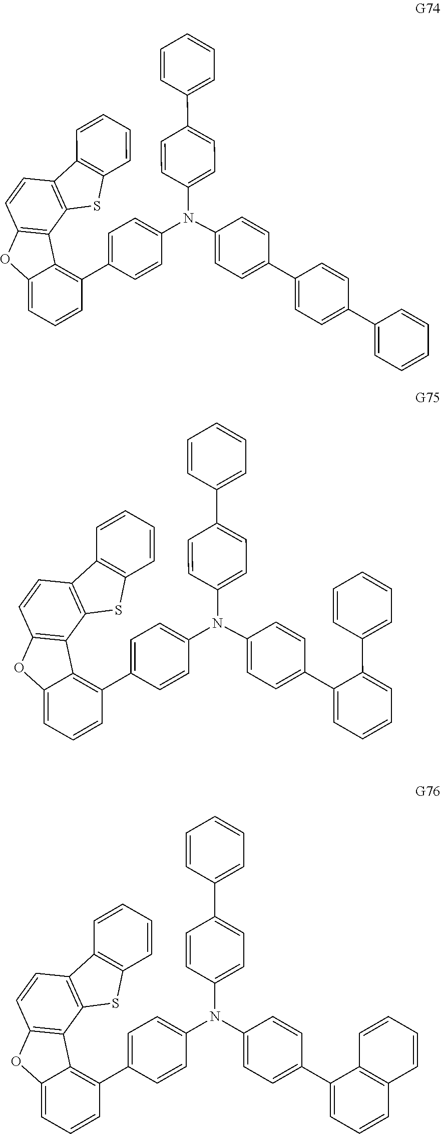 [SMILES: c1ccc(-c2ccc(-c3ccc(N(c4ccc(-c5ccccc5)cc4)c4ccc(-c5cccc6oc7ccc8c9ccccc9sc8c7c56)cc4)cc3)cc2)cc1 ; c1ccc(-c2ccc(N(c3ccc(-c4ccccc4-c4ccccc4)cc3)c3ccc(-c4cccc5oc6ccc7c8ccccc8sc7c6c45)cc3)cc2)cc1 ; c1ccc(-c2ccc(N(c3ccc(-c4cccc5ccccc45)cc3)c3ccc(-c4cccc5oc6ccc7c8ccccc8sc7c6c45)cc3)cc2)cc1]

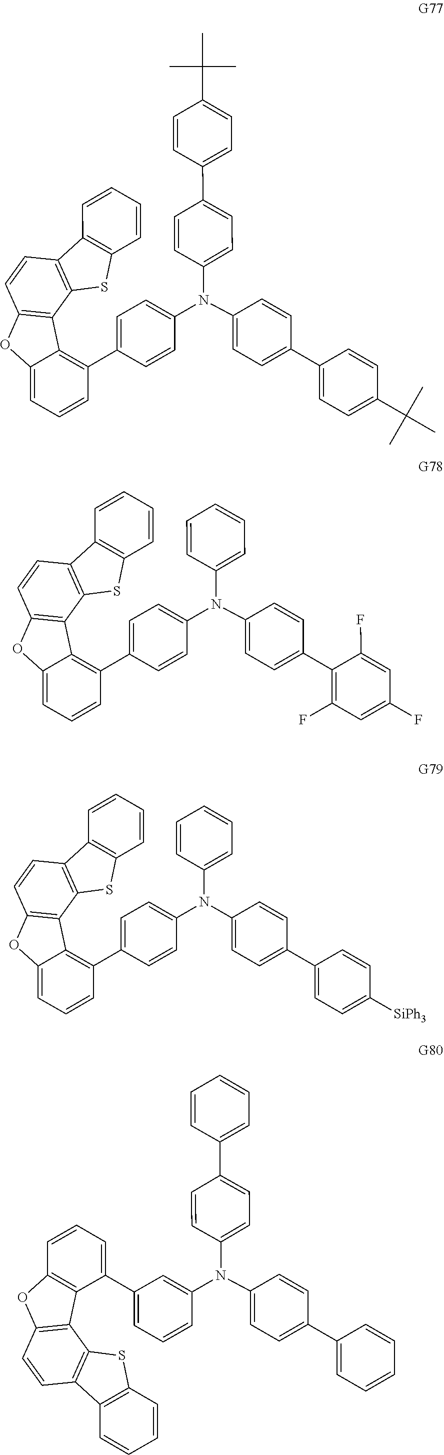

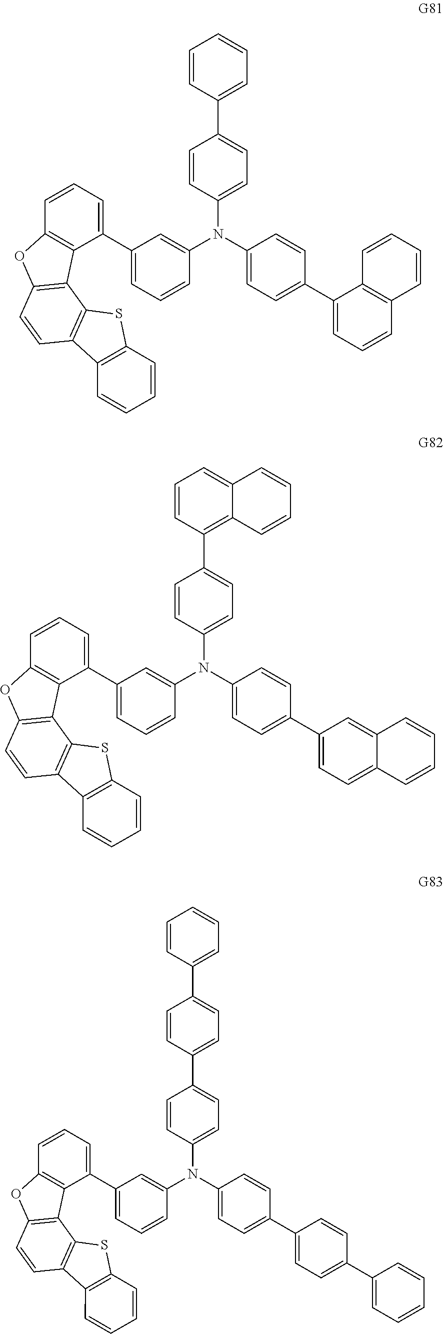 [SMILES: c1ccc(-c2ccc(N(c3ccc(-c4cccc5ccccc45)cc3)c3cccc(-c4cccc5oc6ccc7c8ccccc8sc7c6c45)c3)cc2)cc1 ; c1cc(-c2cccc3oc4ccc5c6ccccc6sc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6oc7ccc8c9ccccc9sc8c7c56)c4)cc3)cc2)cc1]

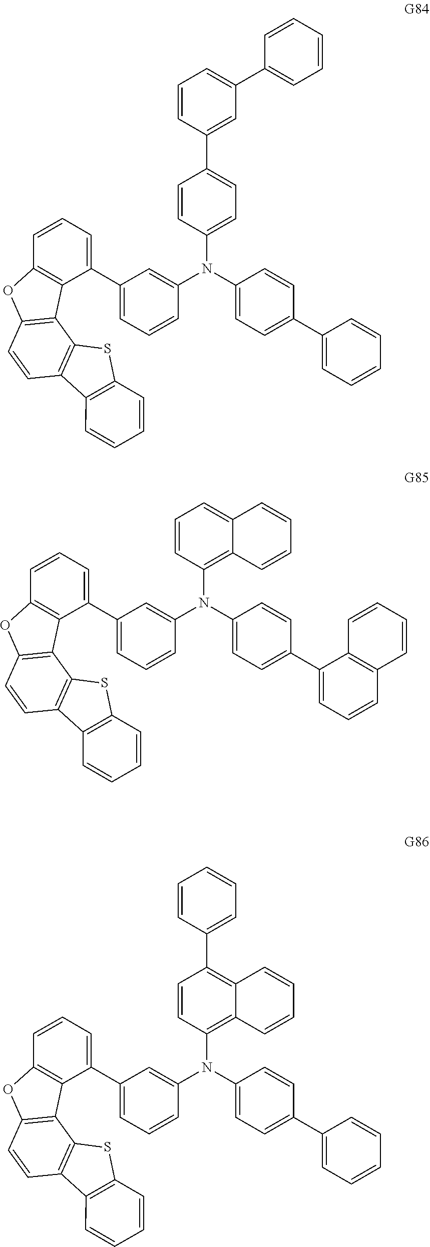 [SMILES: c1ccc(-c2ccc(N(c3ccc(-c4cccc(-c5ccccc5)c4)cc3)c3cccc(-c4cccc5oc6ccc7c8ccccc8sc7c6c45)c3)cc2)cc1 ; c1cc(-c2cccc3oc4ccc5c6ccccc6sc5c4c23)cc(N(c2ccc(-c3cccc4ccccc34)cc2)c2cccc3ccccc23)c1 ; c1ccc(-c2ccc(N(c3cccc(-c4cccc5oc6ccc7c8ccccc8sc7c6c45)c3)c3ccc(-c4ccccc4)c4ccccc34)cc2)cc1]

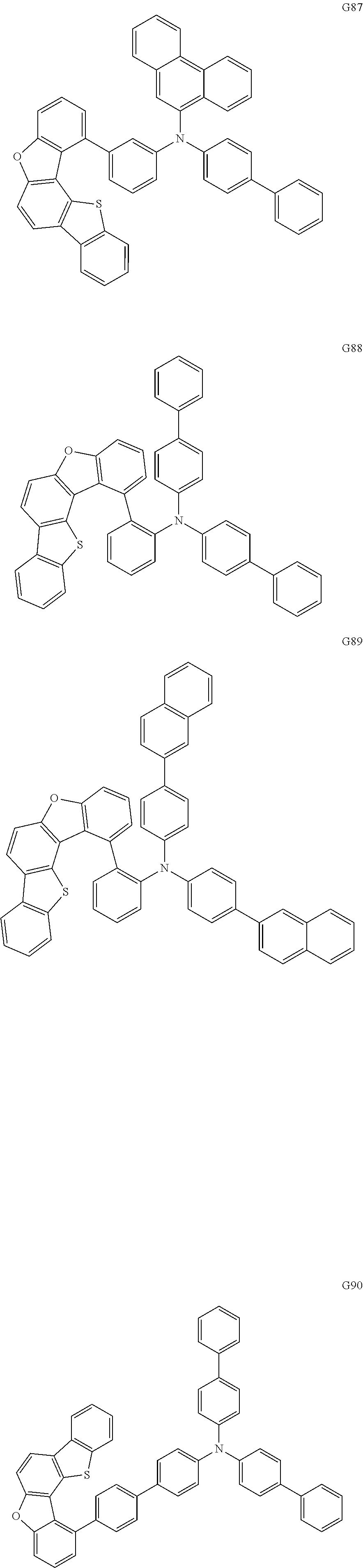 [SMILES: c1ccc(-c2ccc(N(c3cccc(-c4cccc5oc6ccc7c8ccccc8sc7c6c45)c3)c3cc4ccccc4c4ccccc34)cc2)cc1 ; c1ccc(-c2ccc(N(c3ccc(-c4ccccc4)cc3)c3ccccc3-c3cccc4oc5ccc6c7ccccc7sc6c5c34)cc2)cc1 ; c1ccc(N(c2ccc(-c3ccc4ccccc4c3)cc2)c2ccc(-c3ccc4ccccc4c3)cc2)c(-c2cccc3oc4ccc5c6ccccc6sc5c4c23)c1 ; c1ccc(-c2ccc(N(c3ccc(-c4ccccc4)cc3)c3ccc(-c4ccc(-c5cccc6oc7ccc8c9ccccc9sc8c7c56)cc4)cc3)cc2)cc1]

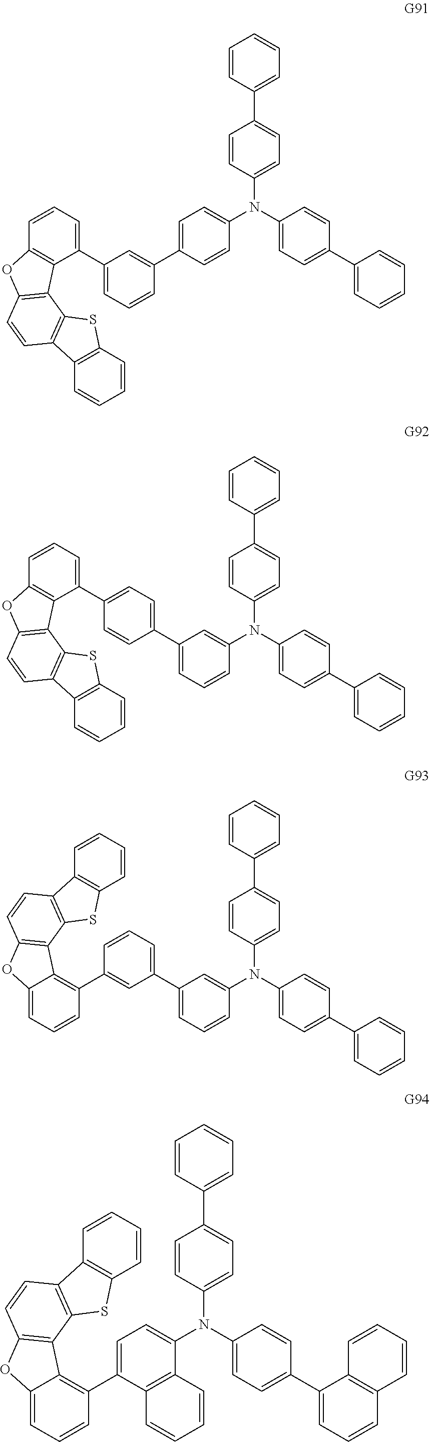 [SMILES: c1ccc(-c2ccc(N(c3ccc(-c4ccccc4)cc3)c3ccc(-c4cccc(-c5cccc6oc7ccc8c9ccccc9sc8c7c56)c4)cc3)cc2)cc1 ; c1ccc(-c2ccc(N(c3ccc(-c4ccccc4)cc3)c3cccc(-c4ccc(-c5cccc6oc7ccc8c9ccccc9sc8c7c56)cc4)c3)cc2)cc1 ; c1ccc(-c2ccc(N(c3ccc(-c4ccccc4)cc3)c3cccc(-c4cccc(-c5cccc6oc7ccc8c9ccccc9sc8c7c56)c4)c3)cc2)cc1 ; c1ccc(-c2ccc(N(c3ccc(-c4cccc5ccccc45)cc3)c3ccc(-c4cccc5oc6ccc7c8ccccc8sc7c6c45)c4ccccc34)cc2)cc1]

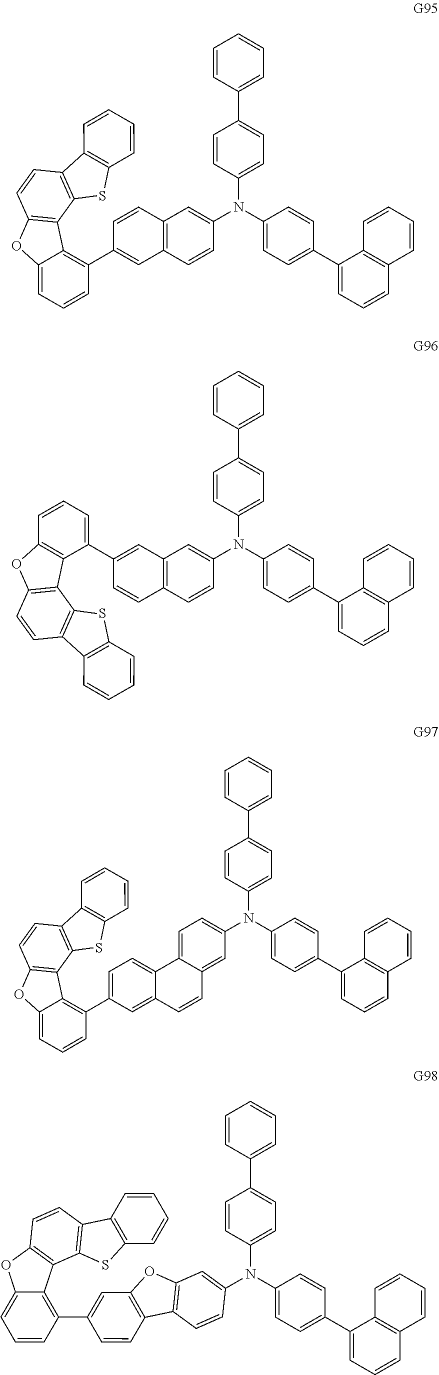 [SMILES: c1ccc(-c2ccc(N(c3ccc(-c4cccc5ccccc45)cc3)c3ccc4cc(-c5cccc6oc7ccc8c9ccccc9sc8c7c56)ccc4c3)cc2)cc1 ; c1ccc(-c2ccc(N(c3ccc(-c4cccc5ccccc45)cc3)c3ccc4ccc(-c5cccc6oc7ccc8c9ccccc9sc8c7c56)cc4c3)cc2)cc1 ; c1ccc(-c2ccc(N(c3ccc(-c4cccc5ccccc45)cc3)c3ccc4c(ccc5cc(-c6cccc7oc8ccc9c%10ccccc%10sc9c8c67)ccc54)c3)cc2)cc1 ; c1ccc(-c2ccc(N(c3ccc(-c4cccc5ccccc45)cc3)c3ccc4c(c3)oc3cc(-c5cccc6oc7ccc8c9ccccc9sc8c7c56)ccc34)cc2)cc1]

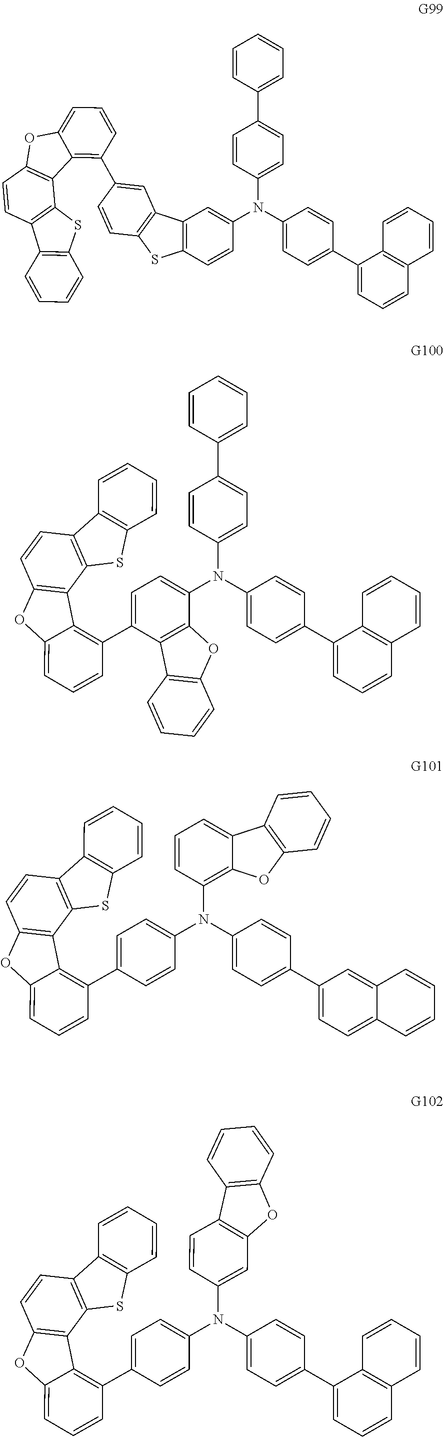 [SMILES: c1ccc(-c2ccc(N(c3ccc(-c4cccc5ccccc45)cc3)c3ccc4sc5ccc(-c6cccc7oc8ccc9c%10ccccc%10sc9c8c67)cc5c4c3)cc2)cc1 ; c1ccc(-c2ccc(N(c3ccc(-c4cccc5ccccc45)cc3)c3ccc(-c4cccc5oc6ccc7c8ccccc8sc7c6c45)c4c3oc3ccccc34)cc2)cc1 ; c1ccc2cc(-c3ccc(N(c4ccc(-c5cccc6oc7ccc8c9ccccc9sc8c7c56)cc4)c4cccc5c4oc4ccccc45)cc3)ccc2c1 ; c1ccc2c(-c3ccc(N(c4ccc(-c5cccc6oc7ccc8c9ccccc9sc8c7c56)cc4)c4ccc5c(c4)oc4ccccc45)cc3)cccc2c1]

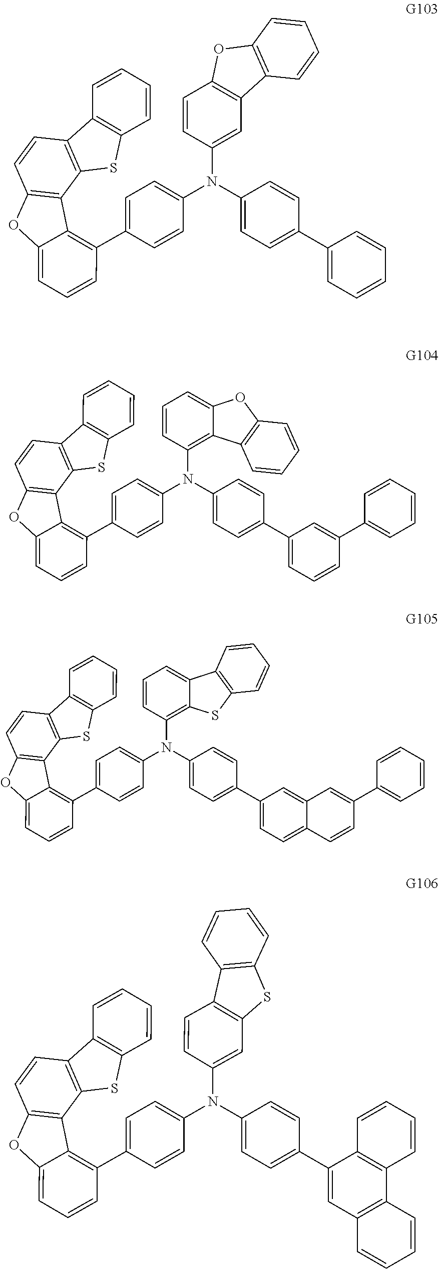 [SMILES: c1ccc(-c2ccc(N(c3ccc(-c4cccc5oc6ccc7c8ccccc8sc7c6c45)cc3)c3ccc4oc5ccccc5c4c3)cc2)cc1 ; c1ccc(-c2cccc(-c3ccc(N(c4ccc(-c5cccc6oc7ccc8c9ccccc9sc8c7c56)cc4)c4cccc5oc6ccccc6c45)cc3)c2)cc1 ; c1ccc(-c2ccc3ccc(-c4ccc(N(c5ccc(-c6cccc7oc8ccc9c%10ccccc%10sc9c8c67)cc5)c5cccc6c5sc5ccccc56)cc4)cc3c2)cc1 ; c1ccc2c(c1)cc(-c1ccc(N(c3ccc(-c4cccc5oc6ccc7c8ccccc8sc7c6c45)cc3)c3ccc4c(c3)sc3ccccc34)cc1)c1ccccc12]

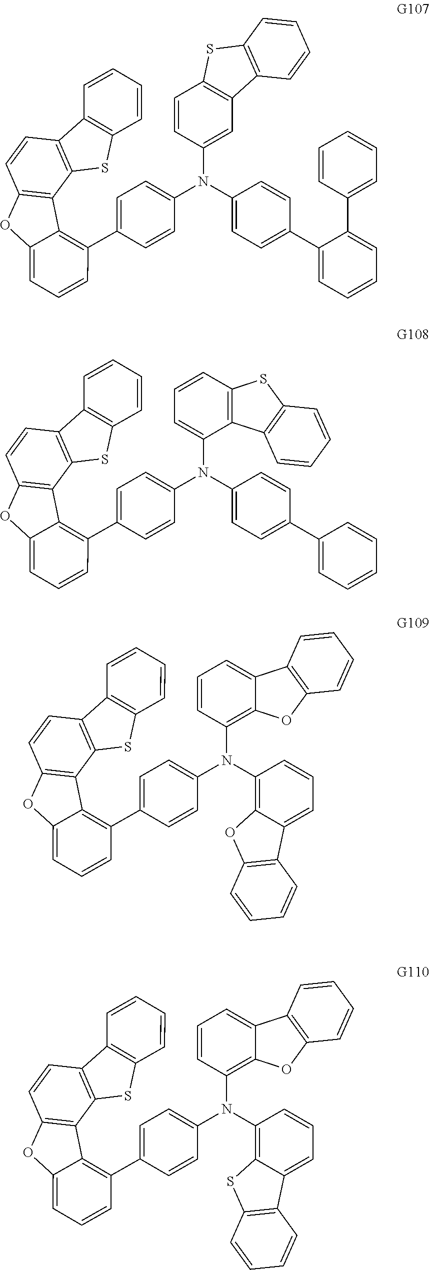

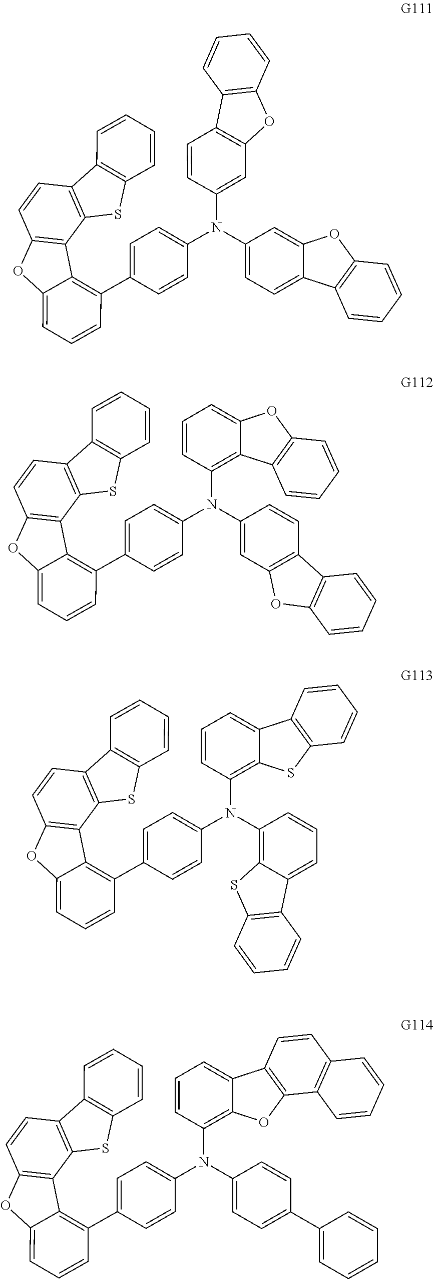 [SMILES: c1ccc2c(c1)oc1cc(N(c3ccc(-c4cccc5oc6ccc7c8ccccc8sc7c6c45)cc3)c3ccc4c(c3)oc3ccccc34)ccc12 ; c1ccc2c(c1)oc1cc(N(c3ccc(-c4cccc5oc6ccc7c8ccccc8sc7c6c45)cc3)c3cccc4oc5ccccc5c34)ccc12 ; c1cc(-c2ccc(N(c3cccc4c3sc3ccccc34)c3cccc4c3sc3ccccc34)cc2)c2c(c1)oc1ccc3c4ccccc4sc3c12 ; c1ccc(-c2ccc(N(c3ccc(-c4cccc5oc6ccc7c8ccccc8sc7c6c45)cc3)c3cccc4c3oc3c5ccccc5ccc43)cc2)cc1]

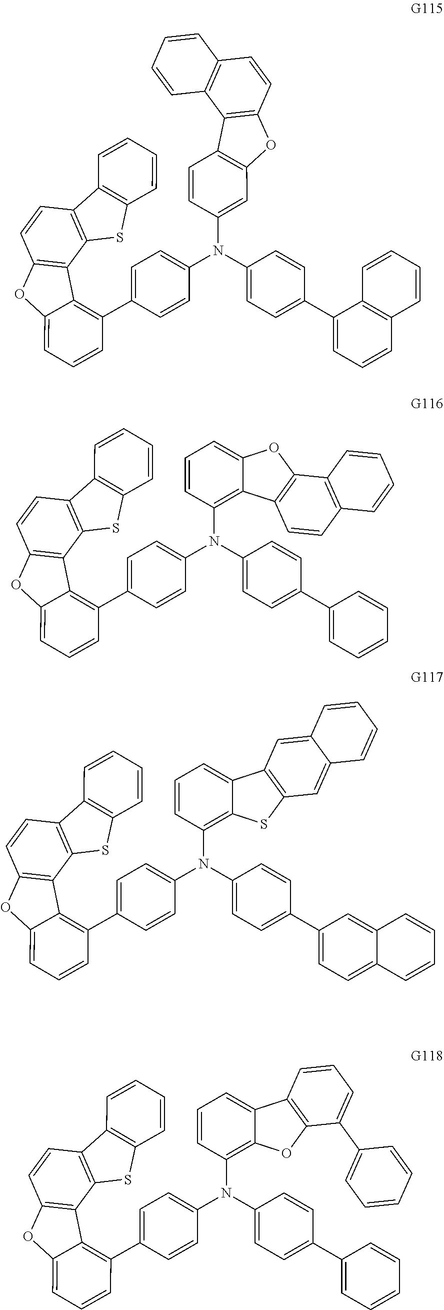

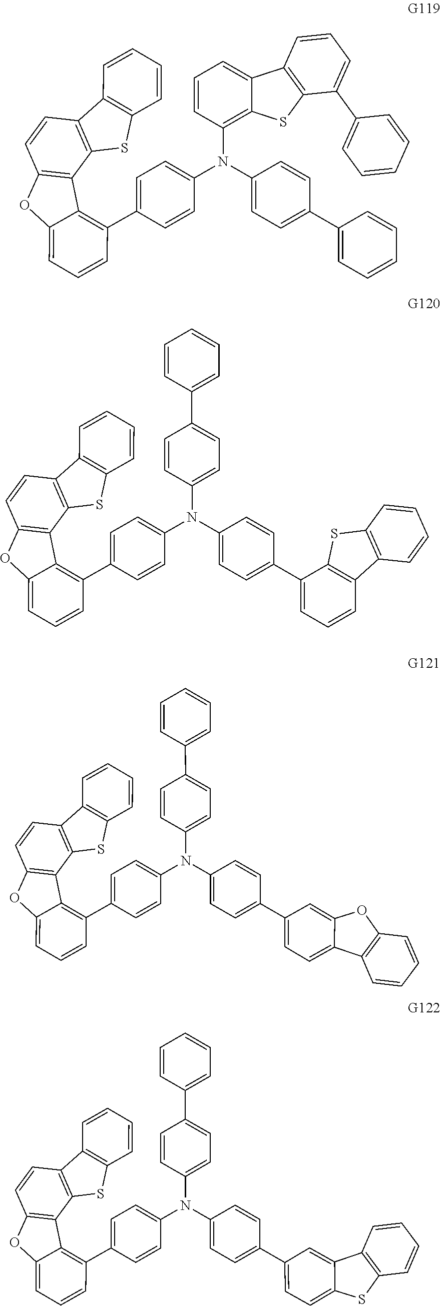 [SMILES: c1ccc(-c2ccc(N(c3ccc(-c4cccc5oc6ccc7c8ccccc8sc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5oc6ccc7c8ccccc8sc7c6c45)cc3)cc2)cc1 ; c1ccc(-c2ccc(N(c3ccc(-c4ccc5c(c4)oc4ccccc45)cc3)c3ccc(-c4cccc5oc6ccc7c8ccccc8sc7c6c45)cc3)cc2)cc1 ; c1ccc(-c2ccc(N(c3ccc(-c4ccc5sc6ccccc6c5c4)cc3)c3ccc(-c4cccc5oc6ccc7c8ccccc8sc7c6c45)cc3)cc2)cc1]

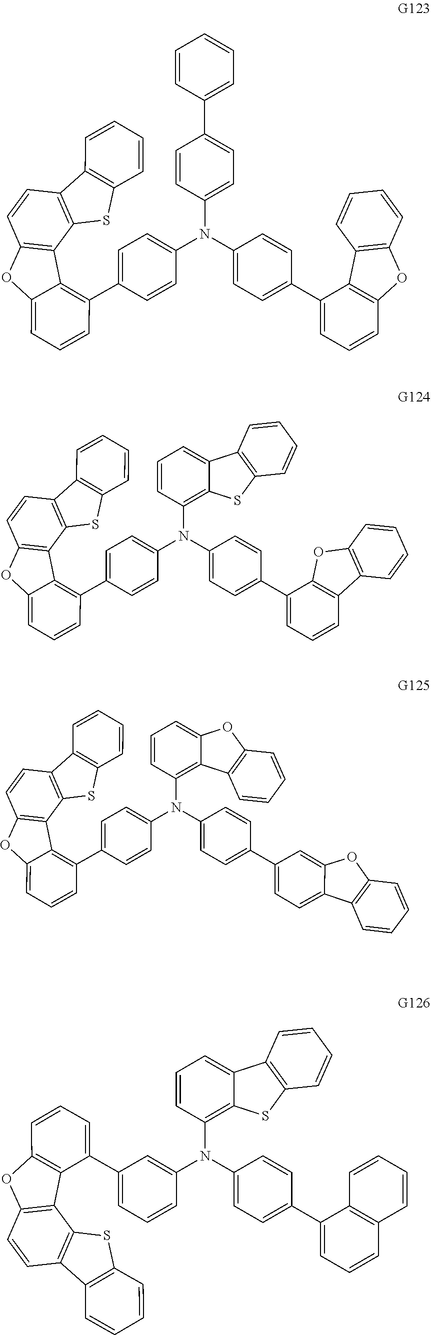 [SMILES: c1ccc(-c2ccc(N(c3ccc(-c4cccc5oc6ccccc6c45)cc3)c3ccc(-c4cccc5oc6ccc7c8ccccc8sc7c6c45)cc3)cc2)cc1 ; c1ccc2c(c1)oc1c(-c3ccc(N(c4ccc(-c5cccc6oc7ccc8c9ccccc9sc8c7c56)cc4)c4cccc5c4sc4ccccc45)cc3)cccc12 ; c1ccc2c(c1)oc1cc(-c3ccc(N(c4ccc(-c5cccc6oc7ccc8c9ccccc9sc8c7c56)cc4)c4cccc5oc6ccccc6c45)cc3)ccc12 ; c1cc(-c2cccc3oc4ccc5c6ccccc6sc5c4c23)cc(N(c2ccc(-c3cccc4ccccc34)cc2)c2cccc3c2sc2ccccc23)c1]

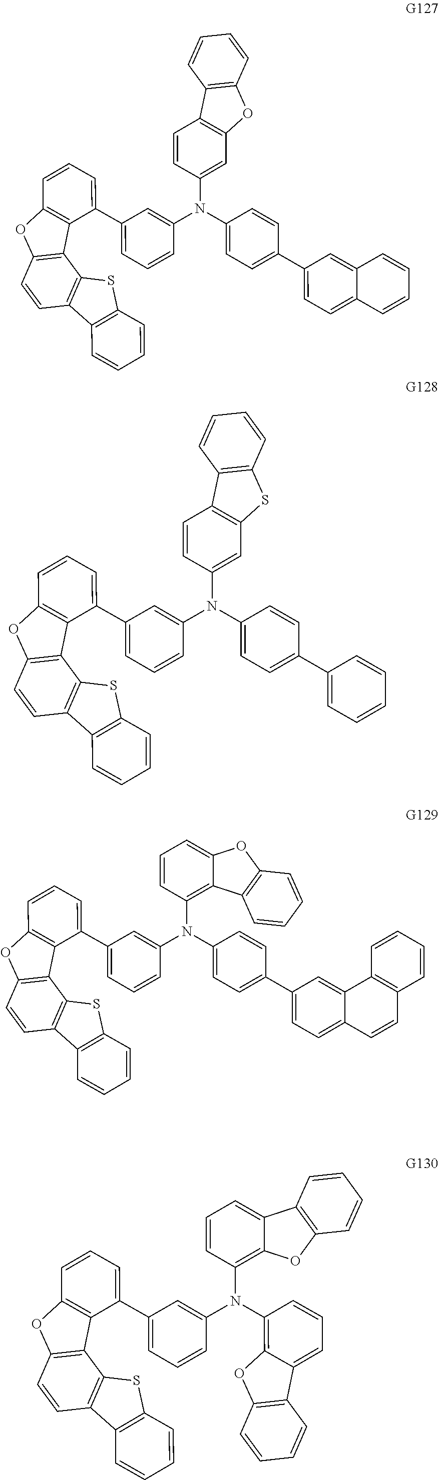 [SMILES: c1cc(-c2cccc3oc4ccc5c6ccccc6sc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5oc6ccc7c8ccccc8sc7c6c45)c3)c3ccc4c(c3)sc3ccccc34)cc2)cc1 ; c1cc(-c2cccc3oc4ccc5c6ccccc6sc5c4c23)cc(N(c2ccc(-c3ccc4ccc5ccccc5c4c3)cc2)c2cccc3oc4ccccc4c23)c1 ; c1cc(-c2cccc3oc4ccc5c6ccccc6sc5c4c23)cc(N(c2cccc3c2oc2ccccc23)c2cccc3c2oc2ccccc23)c1]

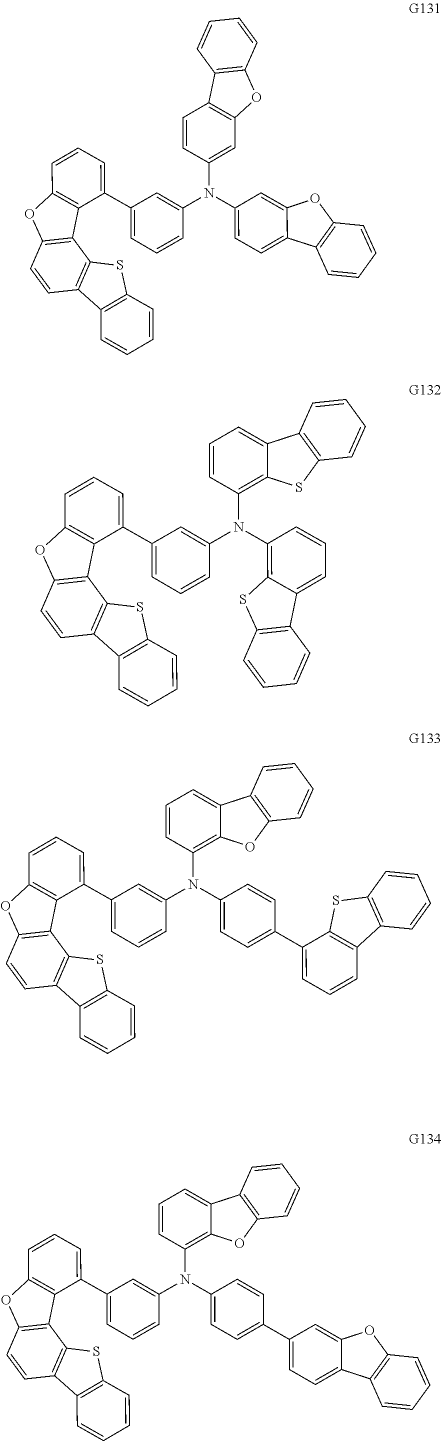 [SMILES: c1cc(-c2cccc3oc4ccc5c6ccccc6sc5c4c23)cc(N(c2ccc3c(c2)oc2ccccc23)c2ccc3c(c2)oc2ccccc23)c1 ; c1cc(-c2cccc3oc4ccc5c6ccccc6sc5c4c23)cc(N(c2cccc3c2sc2ccccc23)c2cccc3c2sc2ccccc23)c1 ; c1cc(-c2cccc3oc4ccc5c6ccccc6sc5c4c23)cc(N(c2ccc(-c3cccc4c3sc3ccccc34)cc2)c2cccc3c2oc2ccccc23)c1 ; c1cc(-c2cccc3oc4ccc5c6ccccc6sc5c4c23)cc(N(c2ccc(-c3ccc4c(c3)oc3ccccc34)cc2)c2cccc3c2oc2ccccc23)c1]

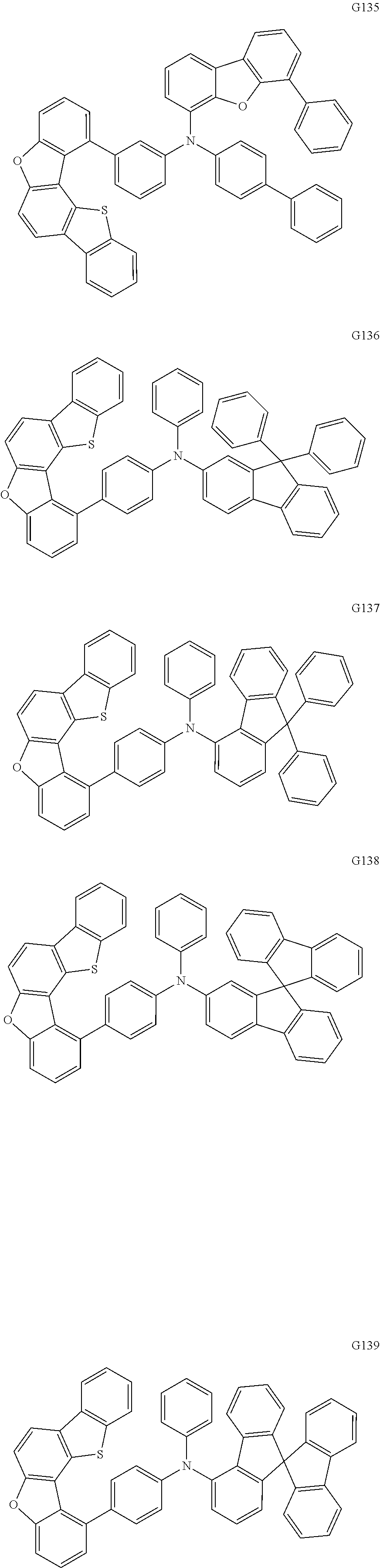 [SMILES: c1ccc(-c2ccc(N(c3cccc(-c4cccc5oc6ccc7c8ccccc8sc7c6c45)c3)c3cccc4c3oc3c(-c5ccccc5)cccc34)cc2)cc1 ; c1ccc(N(c2ccc(-c3cccc4oc5ccc6c7ccccc7sc6c5c34)cc2)c2ccc3c(c2)C(c2ccccc2)(c2ccccc2)c2ccccc2-3)cc1 ; c1ccc(N(c2ccc(-c3cccc4oc5ccc6c7ccccc7sc6c5c34)cc2)c2cccc3c2-c2ccccc2C3(c2ccccc2)c2ccccc2)cc1 ; c1ccc(N(c2ccc(-c3cccc4oc5ccc6c7ccccc7sc6c5c34)cc2)c2ccc3c(c2)C2(c4ccccc4-c4ccccc42)c2ccccc2-3)cc1 ; c1ccc(N(c2ccc(-c3cccc4oc5ccc6c7ccccc7sc6c5c34)cc2)c2cccc3c2-c2ccccc2C32c3ccccc3-c3ccccc32)cc1]

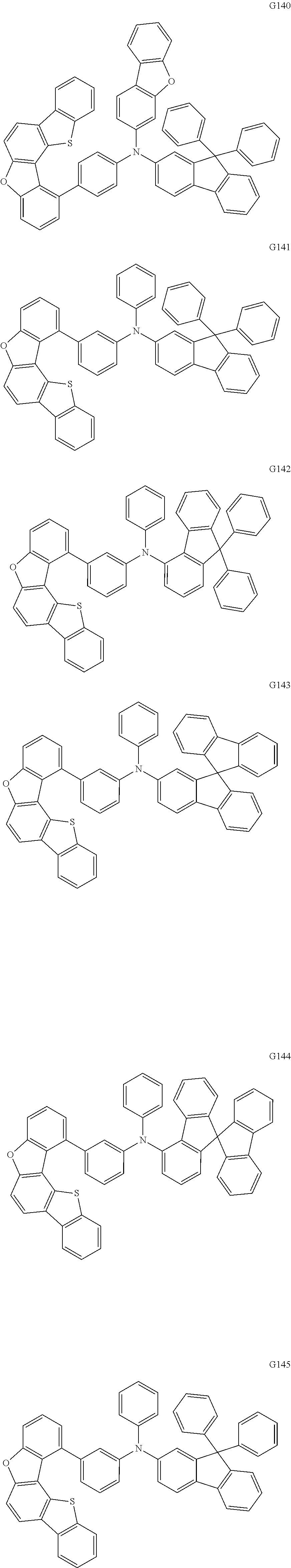

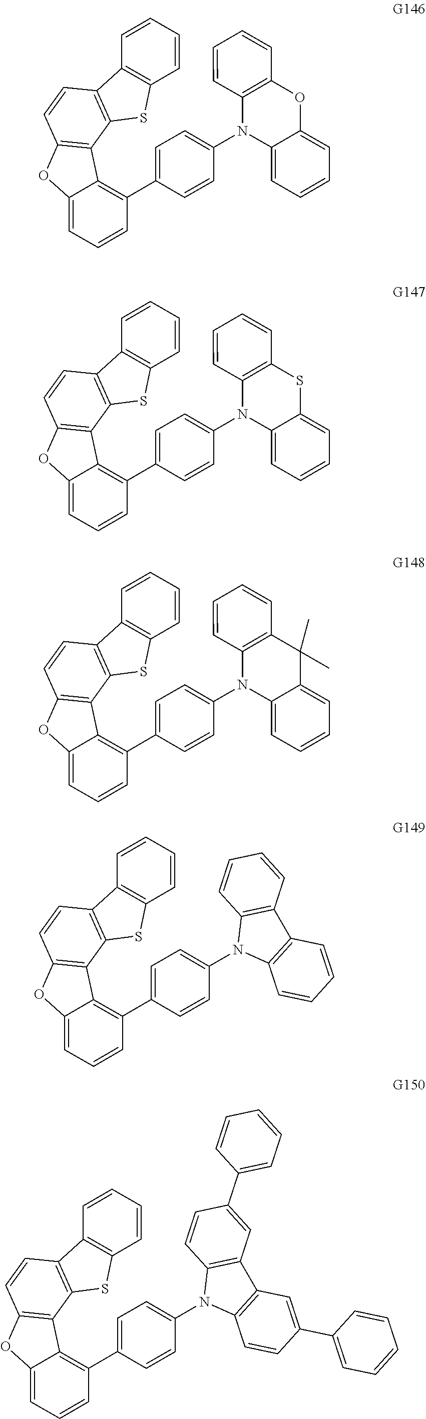 [SMILES: c1ccc2c(c1)Oc1ccccc1N2c1ccc(-c2cccc3oc4ccc5c6ccccc6sc5c4c23)cc1 ; c1ccc2c(c1)Sc1ccccc1N2c1ccc(-c2cccc3oc4ccc5c6ccccc6sc5c4c23)cc1 ; CC1(C)c2ccccc2N(c2ccc(-c3cccc4oc5ccc6c7ccccc7sc6c5c34)cc2)c2ccccc21 ; c1cc(-c2ccc(-n3c4ccccc4c4ccccc43)cc2)c2c(c1)oc1ccc3c4ccccc4sc3c12 ; c1ccc(-c2ccc3c(c2)c2cc(-c4ccccc4)ccc2n3-c2ccc(-c3cccc4oc5ccc6c7ccccc7sc6c5c34)cc2)cc1]

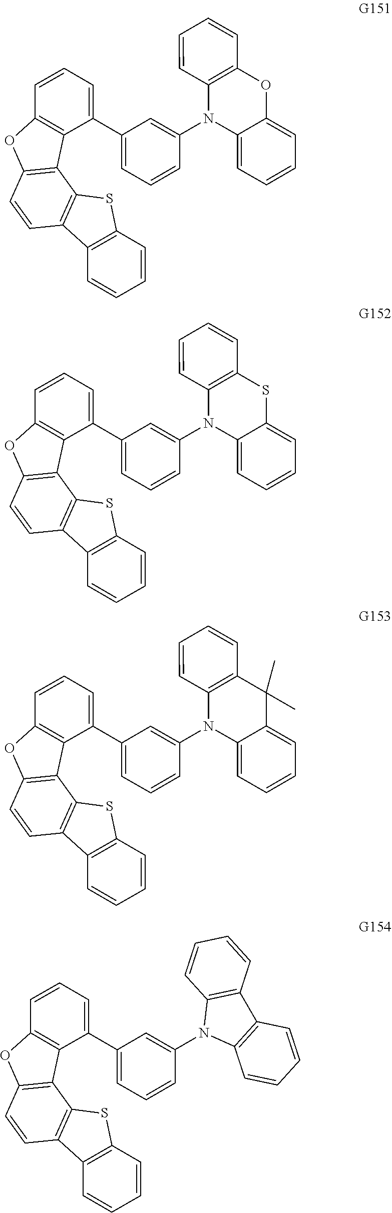 [SMILES: c1cc(-c2cccc3oc4ccc5c6ccccc6sc5c4c23)cc(N2c3ccccc3Oc3ccccc32)c1 ; c1cc(-c2cccc3oc4ccc5c6ccccc6sc5c4c23)cc(N2c3ccccc3Sc3ccccc32)c1 ; CC1(C)c2ccccc2N(c2cccc(-c3cccc4oc5ccc6c7ccccc7sc6c5c34)c2)c2ccccc21 ; c1cc(-c2cccc3oc4ccc5c6ccccc6sc5c4c23)cc(-n2c3ccccc3c3ccccc32)c1]

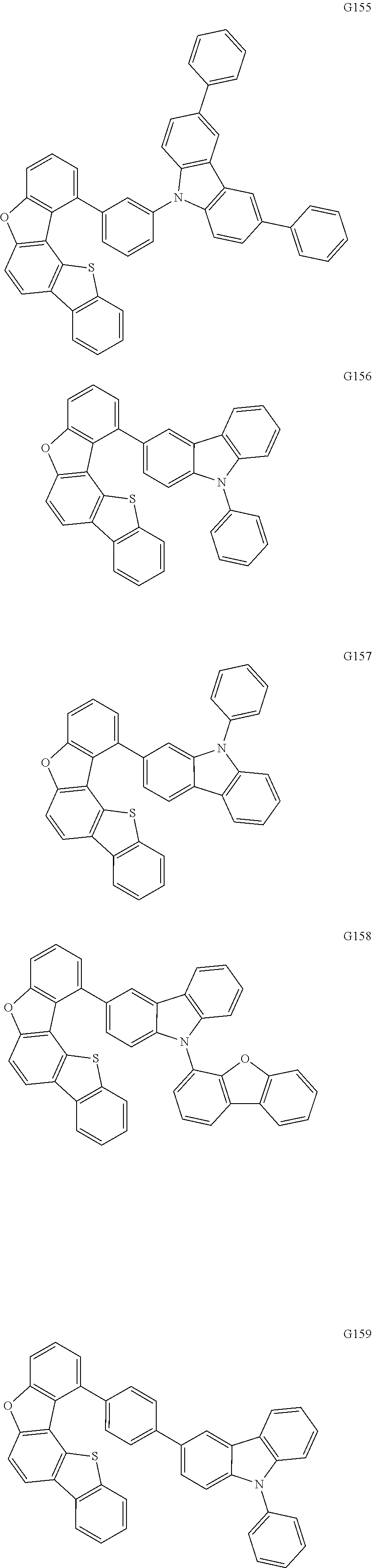

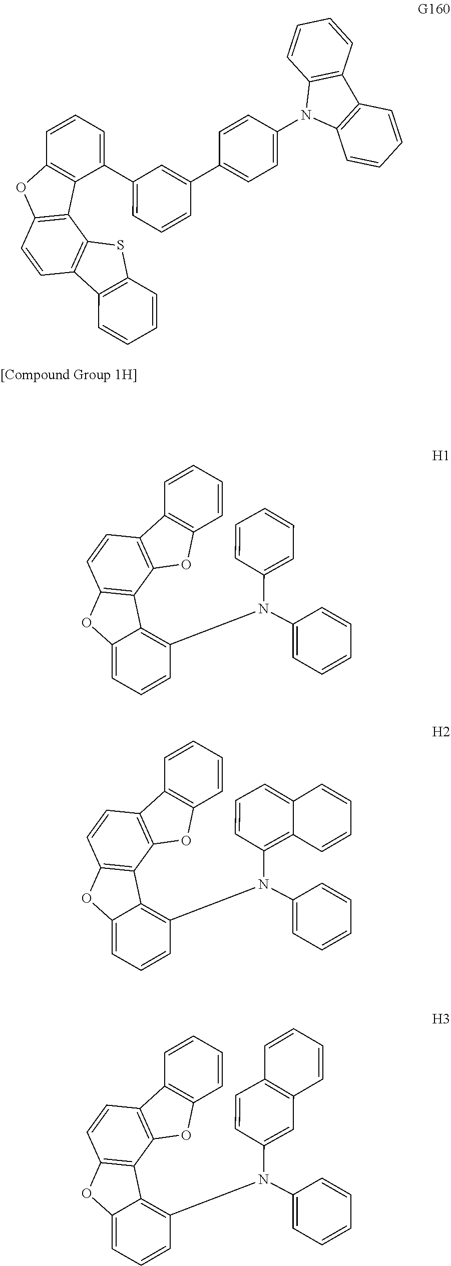 [SMILES: c1cc(-c2ccc(-n3c4ccccc4c4ccccc43)cc2)cc(-c2cccc3oc4ccc5c6ccccc6sc5c4c23)c1 ; c1ccc(N(c2ccccc2)c2cccc3oc4ccc5c6ccccc6oc5c4c23)cc1 ; c1ccc(N(c2cccc3ccccc23)c2cccc3oc4ccc5c6ccccc6oc5c4c23)cc1 ; c1ccc(N(c2ccc3ccccc3c2)c2cccc3oc4ccc5c6ccccc6oc5c4c23)cc1]

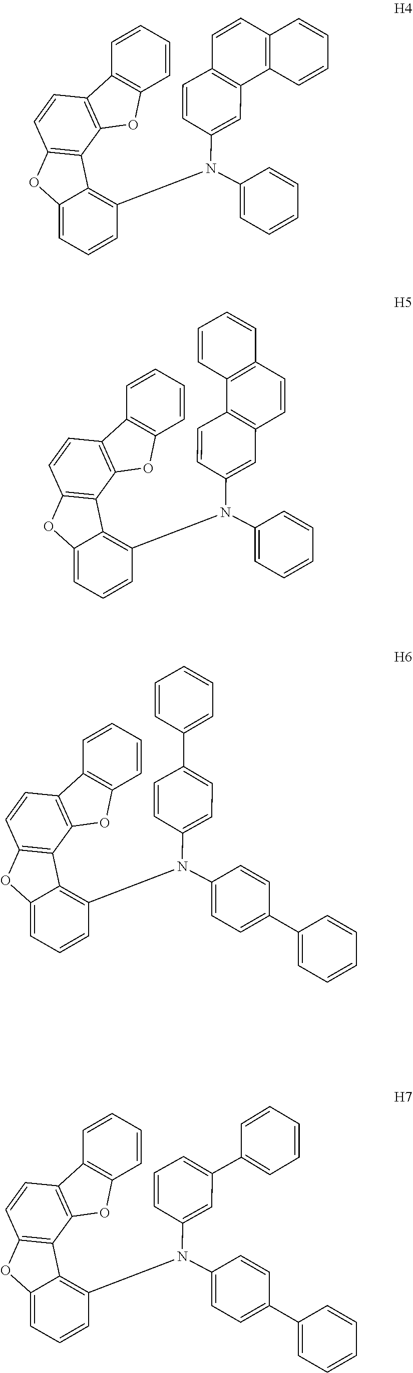 [SMILES: c1ccc(N(c2ccc3ccc4ccccc4c3c2)c2cccc3oc4ccc5c6ccccc6oc5c4c23)cc1 ; c1ccc(N(c2ccc3c(ccc4ccccc43)c2)c2cccc3oc4ccc5c6ccccc6oc5c4c23)cc1 ; c1ccc(-c2ccc(N(c3ccc(-c4ccccc4)cc3)c3cccc4oc5ccc6c7ccccc7oc6c5c34)cc2)cc1 ; c1ccc(-c2ccc(N(c3cccc(-c4ccccc4)c3)c3cccc4oc5ccc6c7ccccc7oc6c5c34)cc2)cc1]

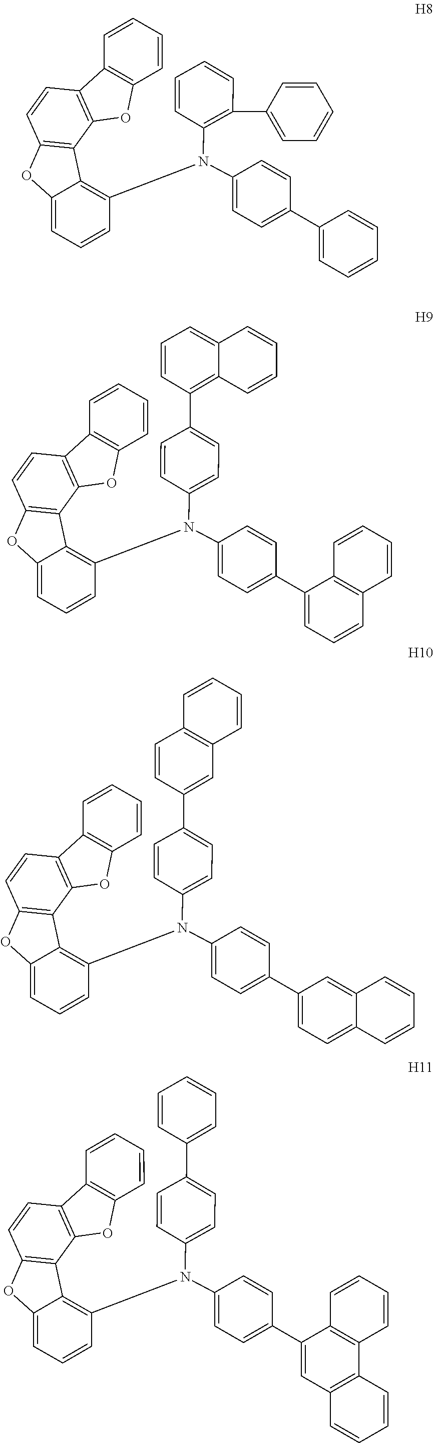 [SMILES: c1ccc(-c2ccc(N(c3ccccc3-c3ccccc3)c3cccc4oc5ccc6c7ccccc7oc6c5c34)cc2)cc1 ; c1ccc2c(-c3ccc(N(c4ccc(-c5cccc6ccccc56)cc4)c4cccc5oc6ccc7c8ccccc8oc7c6c45)cc3)cccc2c1 ; c1ccc2cc(-c3ccc(N(c4ccc(-c5ccc6ccccc6c5)cc4)c4cccc5oc6ccc7c8ccccc8oc7c6c45)cc3)ccc2c1 ; c1ccc(-c2ccc(N(c3ccc(-c4cc5ccccc5c5ccccc45)cc3)c3cccc4oc5ccc6c7ccccc7oc6c5c34)cc2)cc1]

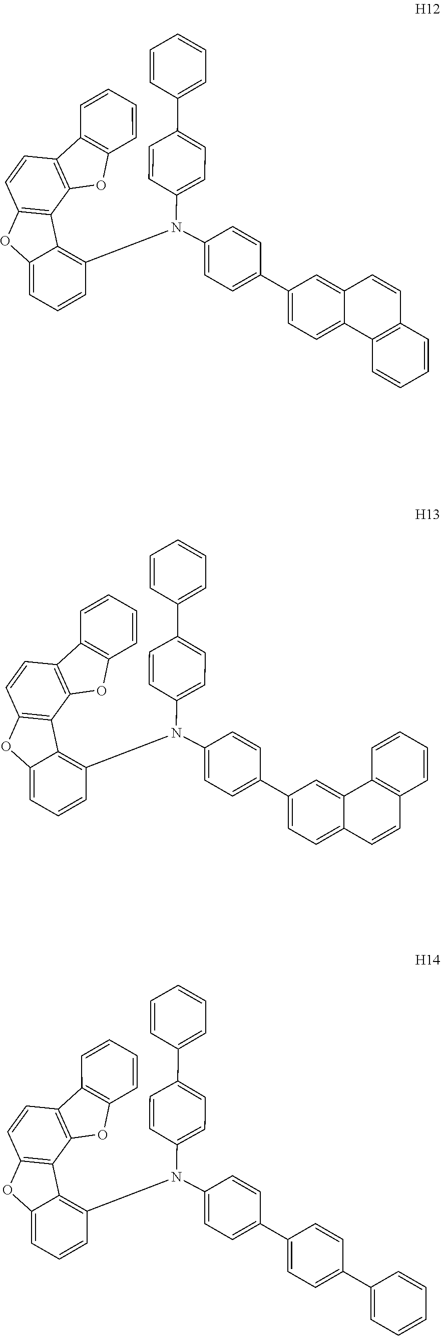 [SMILES: c1ccc(-c2ccc(N(c3ccc(-c4ccc5c(ccc6ccccc65)c4)cc3)c3cccc4oc5ccc6c7ccccc7oc6c5c34)cc2)cc1 ; c1ccc(-c2ccc(N(c3ccc(-c4ccc5ccc6ccccc6c5c4)cc3)c3cccc4oc5ccc6c7ccccc7oc6c5c34)cc2)cc1 ; c1ccc(-c2ccc(-c3ccc(N(c4ccc(-c5ccccc5)cc4)c4cccc5oc6ccc7c8ccccc8oc7c6c45)cc3)cc2)cc1]

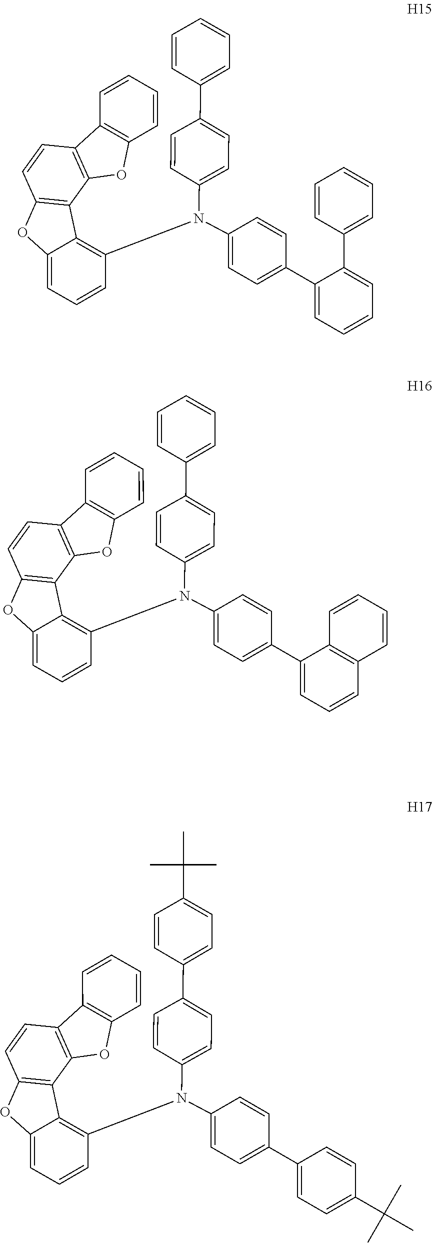 [SMILES: c1ccc(-c2ccc(N(c3ccc(-c4ccccc4-c4ccccc4)cc3)c3cccc4oc5ccc6c7ccccc7oc6c5c34)cc2)cc1 ; c1ccc(-c2ccc(N(c3ccc(-c4cccc5ccccc45)cc3)c3cccc4oc5ccc6c7ccccc7oc6c5c34)cc2)cc1 ; CC(C)(C)c1ccc(-c2ccc(N(c3ccc(-c4ccc(C(C)(C)C)cc4)cc3)c3cccc4oc5ccc6c7ccccc7oc6c5c34)cc2)cc1]

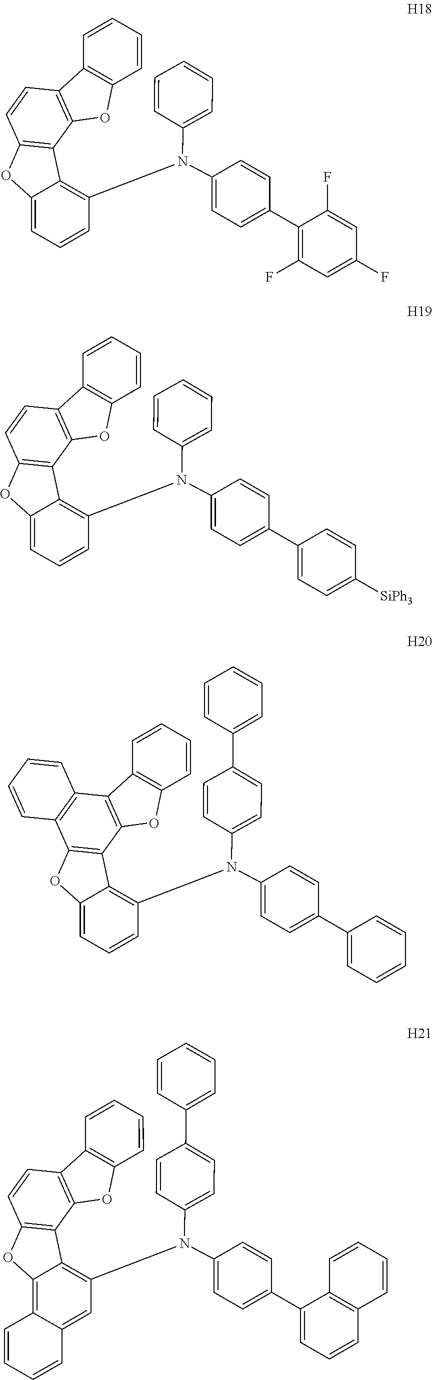

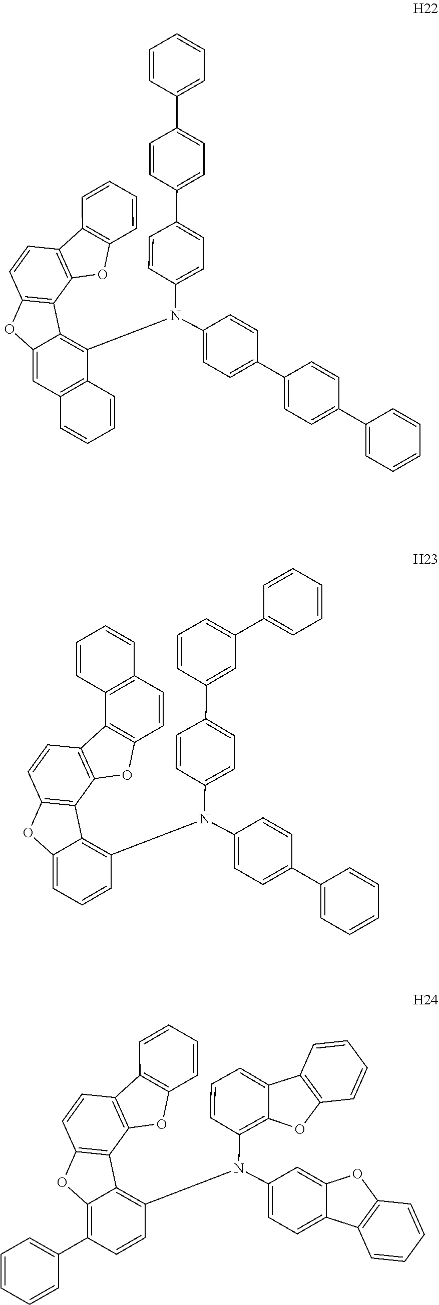 [SMILES: c1ccc(-c2ccc(-c3ccc(N(c4ccc(-c5ccc(-c6ccccc6)cc5)cc4)c4c5ccccc5cc5oc6ccc7c8ccccc8oc7c6c45)cc3)cc2)cc1 ; c1ccc(-c2ccc(N(c3ccc(-c4cccc(-c5ccccc5)c4)cc3)c3cccc4oc5ccc6c(oc7ccc8ccccc8c76)c5c34)cc2)cc1 ; c1ccc(-c2ccc(N(c3ccc4c(c3)oc3ccccc34)c3cccc4c3oc3ccccc34)c3c2oc2ccc4c5ccccc5oc4c23)cc1]

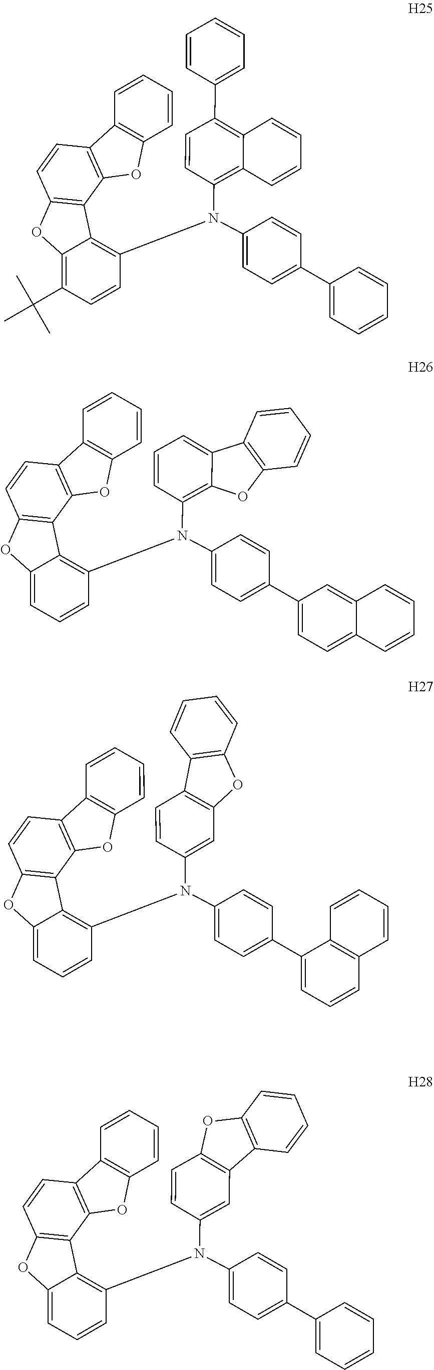 [SMILES: CC(C)(C)c1ccc(N(c2ccc(-c3ccccc3)cc2)c2ccc(-c3ccccc3)c3ccccc23)c2c1oc1ccc3c4ccccc4oc3c12 ; c1ccc2cc(-c3ccc(N(c4cccc5c4oc4ccccc45)c4cccc5oc6ccc7c8ccccc8oc7c6c45)cc3)ccc2c1 ; c1ccc2c(-c3ccc(N(c4ccc5c(c4)oc4ccccc45)c4cccc5oc6ccc7c8ccccc8oc7c6c45)cc3)cccc2c1 ; c1ccc(-c2ccc(N(c3ccc4oc5ccccc5c4c3)c3cccc4oc5ccc6c7ccccc7oc6c5c34)cc2)cc1]

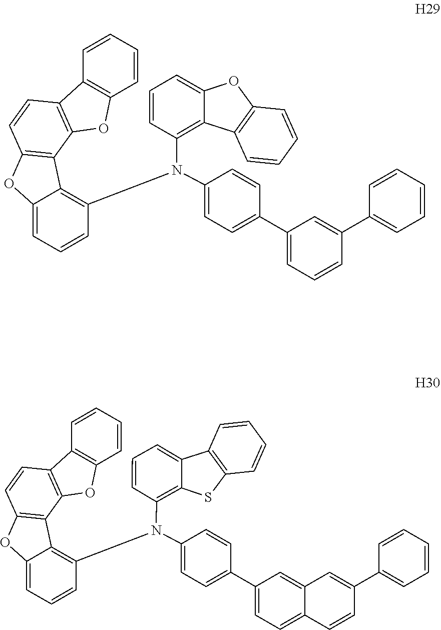 [SMILES: c1ccc(-c2cccc(-c3ccc(N(c4cccc5oc6ccccc6c45)c4cccc5oc6ccc7c8ccccc8oc7c6c45)cc3)c2)cc1 ; c1ccc(-c2ccc3ccc(-c4ccc(N(c5cccc6c5sc5ccccc56)c5cccc6oc7ccc8c9ccccc9oc8c7c56)cc4)cc3c2)cc1]

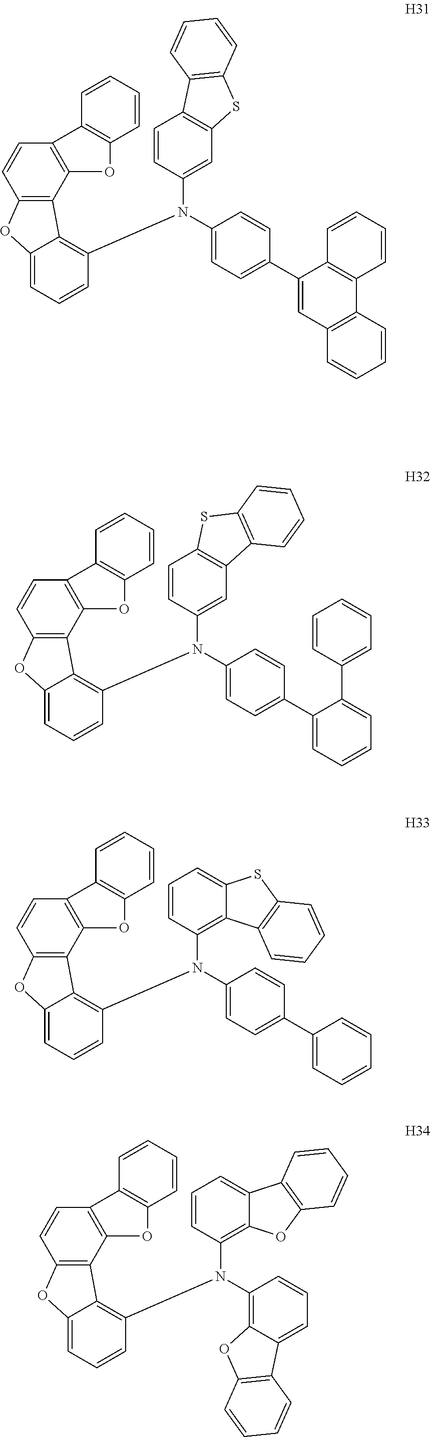 [SMILES: c1ccc2c(c1)cc(-c1ccc(N(c3ccc4c(c3)sc3ccccc34)c3cccc4oc5ccc6c7ccccc7oc6c5c34)cc1)c1ccccc12 ; c1ccc(-c2ccccc2-c2ccc(N(c3ccc4sc5ccccc5c4c3)c3cccc4oc5ccc6c7ccccc7oc6c5c34)cc2)cc1 ; c1ccc(-c2ccc(N(c3cccc4sc5ccccc5c34)c3cccc4oc5ccc6c7ccccc7oc6c5c34)cc2)cc1 ; c1ccc2c(c1)oc1c(N(c3cccc4c3oc3ccccc34)c3cccc4oc5ccc6c7ccccc7oc6c5c34)cccc12]

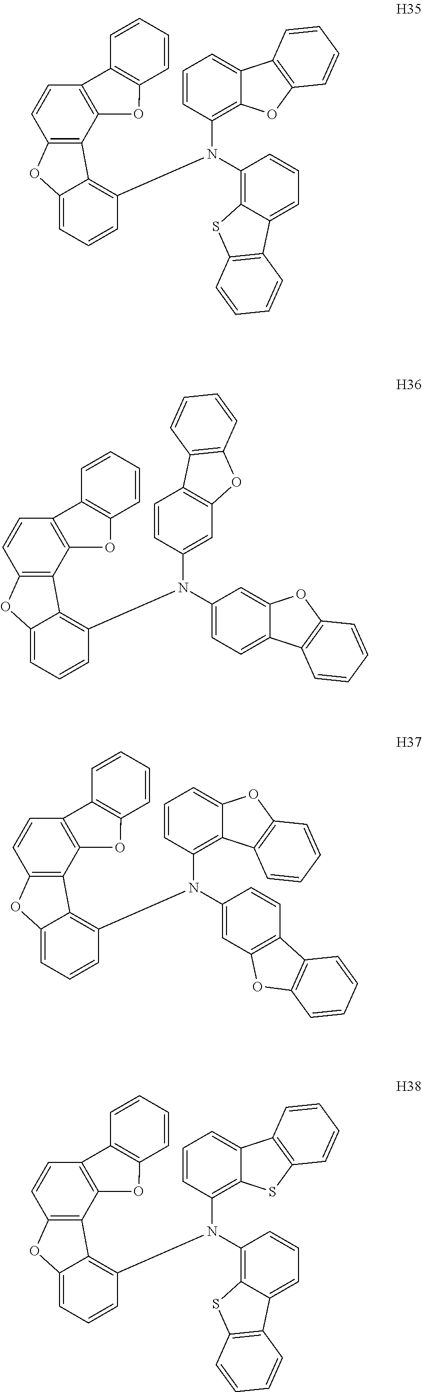 [SMILES: c1ccc2c(c1)oc1c(N(c3cccc4c3sc3ccccc34)c3cccc4oc5ccc6c7ccccc7oc6c5c34)cccc12 ; c1ccc2c(c1)oc1cc(N(c3ccc4c(c3)oc3ccccc34)c3cccc4oc5ccc6c7ccccc7oc6c5c34)ccc12 ; c1ccc2c(c1)oc1cc(N(c3cccc4oc5ccccc5c34)c3cccc4oc5ccc6c7ccccc7oc6c5c34)ccc12 ; c1ccc2c(c1)oc1c2ccc2oc3cccc(N(c4cccc5c4sc4ccccc45)c4cccc5c4sc4ccccc45)c3c21]

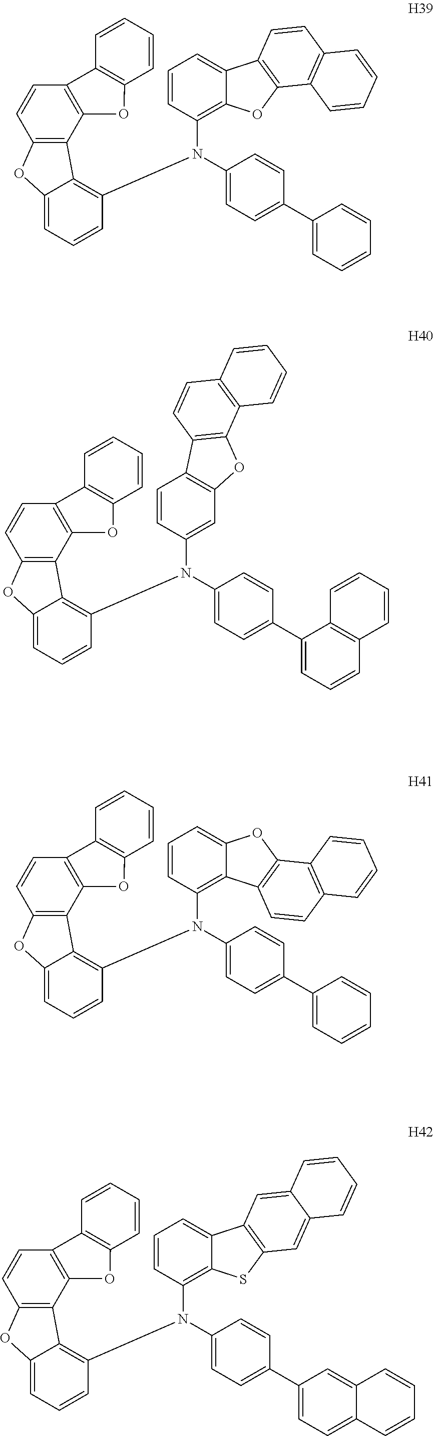 [SMILES: c1ccc(-c2ccc(N(c3cccc4c3oc3c5ccccc5ccc43)c3cccc4oc5ccc6c7ccccc7oc6c5c34)cc2)cc1 ; c1ccc2c(-c3ccc(N(c4ccc5c(c4)oc4c6ccccc6ccc54)c4cccc5oc6ccc7c8ccccc8oc7c6c45)cc3)cccc2c1 ; c1ccc(-c2ccc(N(c3cccc4oc5c6ccccc6ccc5c34)c3cccc4oc5ccc6c7ccccc7oc6c5c34)cc2)cc1 ; c1ccc2cc(-c3ccc(N(c4cccc5c4sc4cc6ccccc6cc45)c4cccc5oc6ccc7c8ccccc8oc7c6c45)cc3)ccc2c1]

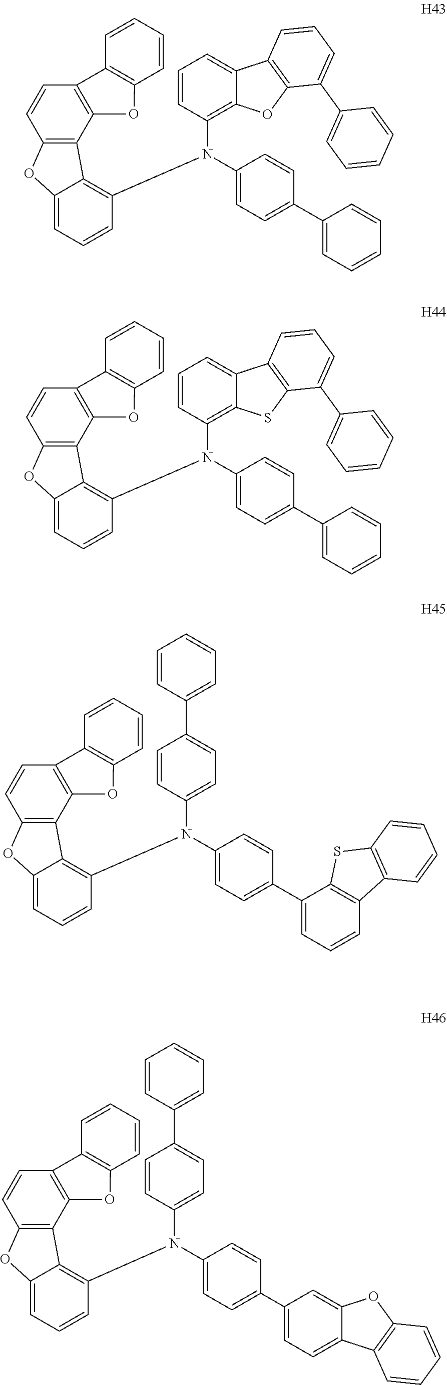 [SMILES: c1ccc(-c2ccc(N(c3cccc4c3oc3c(-c5ccccc5)cccc34)c3cccc4oc5ccc6c7ccccc7oc6c5c34)cc2)cc1 ; c1ccc(-c2ccc(N(c3cccc4c3sc3c(-c5ccccc5)cccc34)c3cccc4oc5ccc6c7ccccc7oc6c5c34)cc2)cc1 ; c1ccc(-c2ccc(N(c3ccc(-c4cccc5c4sc4ccccc45)cc3)c3cccc4oc5ccc6c7ccccc7oc6c5c34)cc2)cc1 ; c1ccc(-c2ccc(N(c3ccc(-c4ccc5c(c4)oc4ccccc45)cc3)c3cccc4oc5ccc6c7ccccc7oc6c5c34)cc2)cc1]

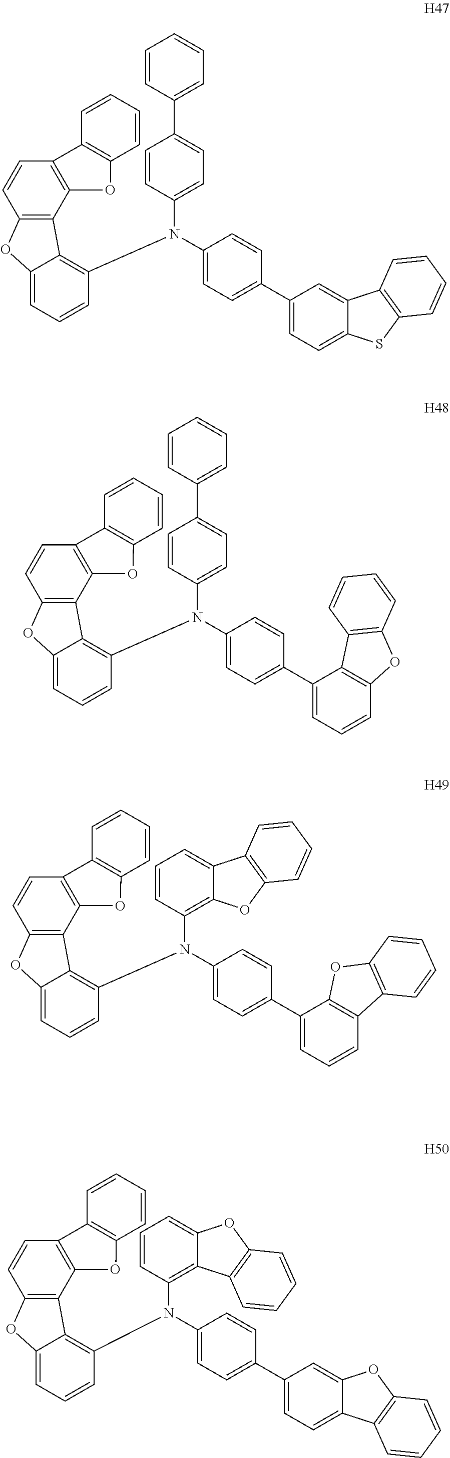 [SMILES: c1ccc(-c2ccc(N(c3ccc(-c4ccc5sc6ccccc6c5c4)cc3)c3cccc4oc5ccc6c7ccccc7oc6c5c34)cc2)cc1 ; c1ccc(-c2ccc(N(c3ccc(-c4cccc5oc6ccccc6c45)cc3)c3cccc4oc5ccc6c7ccccc7oc6c5c34)cc2)cc1 ; c1ccc2c(c1)oc1c(-c3ccc(N(c4cccc5c4oc4ccccc45)c4cccc5oc6ccc7c8ccccc8oc7c6c45)cc3)cccc12 ; c1ccc2c(c1)oc1cc(-c3ccc(N(c4cccc5oc6ccccc6c45)c4cccc5oc6ccc7c8ccccc8oc7c6c45)cc3)ccc12]

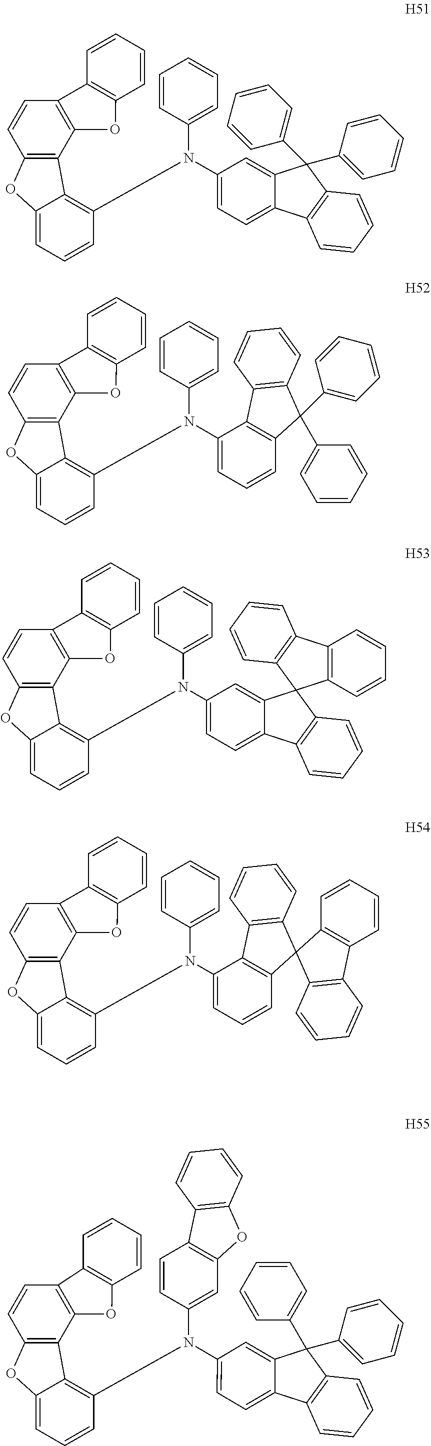 [SMILES: c1ccc(N(c2ccc3c(c2)C(c2ccccc2)(c2ccccc2)c2ccccc2-3)c2cccc3oc4ccc5c6ccccc6oc5c4c23)cc1 ; c1ccc(N(c2cccc3c2-c2ccccc2C3(c2ccccc2)c2ccccc2)c2cccc3oc4ccc5c6ccccc6oc5c4c23)cc1 ; c1ccc(N(c2ccc3c(c2)C2(c4ccccc4-c4ccccc42)c2ccccc2-3)c2cccc3oc4ccc5c6ccccc6oc5c4c23)cc1 ; c1ccc(N(c2cccc3c2-c2ccccc2C32c3ccccc3-c3ccccc32)c2cccc3oc4ccc5c6ccccc6oc5c4c23)cc1 ; c1ccc(C2(c3ccccc3)c3ccccc3-c3ccc(N(c4ccc5c(c4)oc4ccccc45)c4cccc5oc6ccc7c8ccccc8oc7c6c45)cc32)cc1]

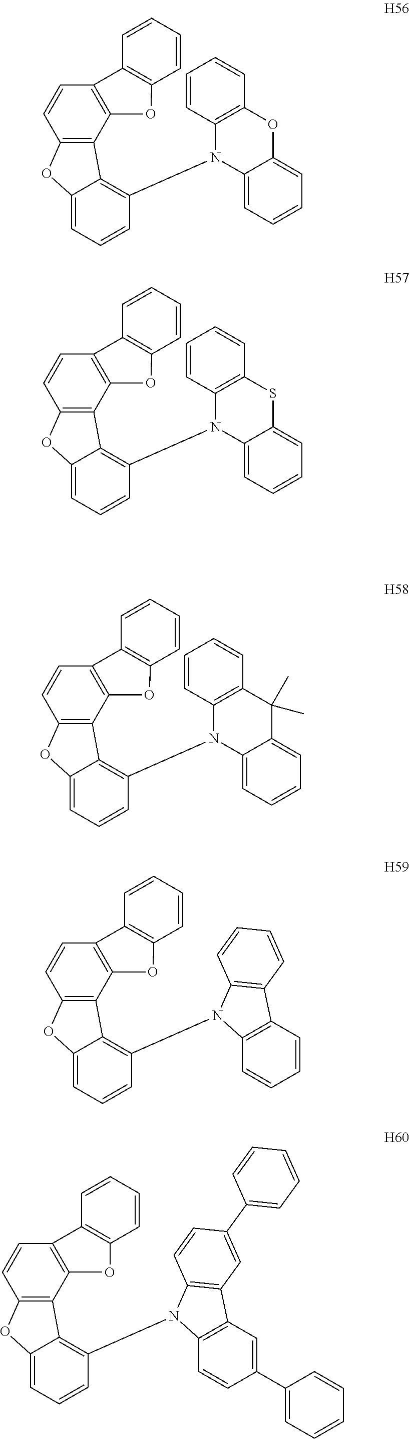 [SMILES: c1ccc2c(c1)Oc1ccccc1N2c1cccc2oc3ccc4c5ccccc5oc4c3c12 ; c1ccc2c(c1)Sc1ccccc1N2c1cccc2oc3ccc4c5ccccc5oc4c3c12 ; CC1(C)c2ccccc2N(c2cccc3oc4ccc5c6ccccc6oc5c4c23)c2ccccc21 ; c1ccc2c(c1)oc1c2ccc2oc3cccc(-n4c5ccccc5c5ccccc54)c3c21 ; c1ccc(-c2ccc3c(c2)c2cc(-c4ccccc4)ccc2n3-c2cccc3oc4ccc5c6ccccc6oc5c4c23)cc1]

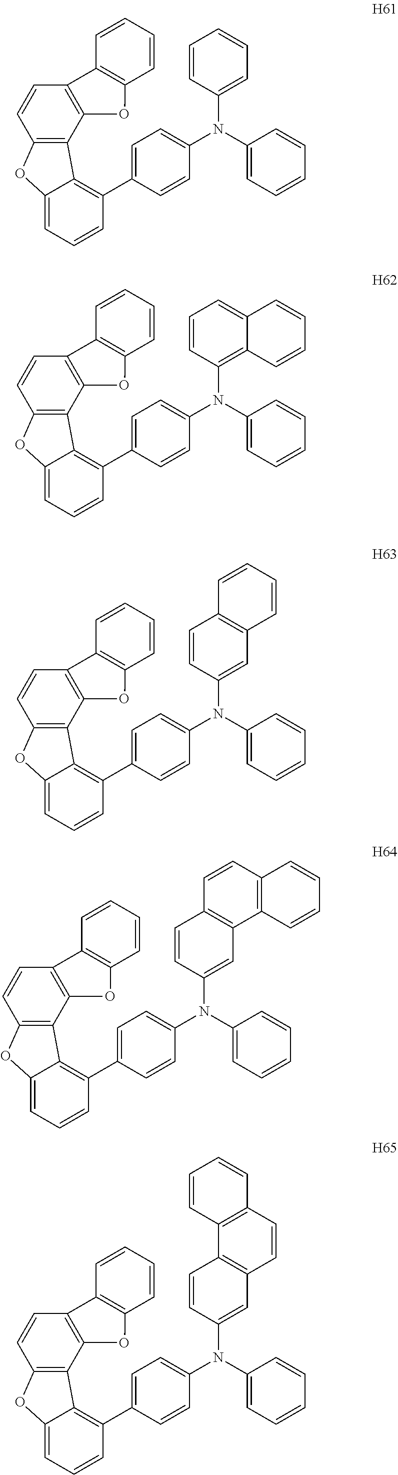 [SMILES: c1ccc(N(c2ccccc2)c2ccc(-c3cccc4oc5ccc6c7ccccc7oc6c5c34)cc2)cc1 ; c1ccc(N(c2ccc(-c3cccc4oc5ccc6c7ccccc7oc6c5c34)cc2)c2cccc3ccccc23)cc1 ; c1ccc(N(c2ccc(-c3cccc4oc5ccc6c7ccccc7oc6c5c34)cc2)c2ccc3ccccc3c2)cc1 ; c1ccc(N(c2ccc(-c3cccc4oc5ccc6c7ccccc7oc6c5c34)cc2)c2ccc3ccc4ccccc4c3c2)cc1 ; c1ccc(N(c2ccc(-c3cccc4oc5ccc6c7ccccc7oc6c5c34)cc2)c2ccc3c(ccc4ccccc43)c2)cc1]

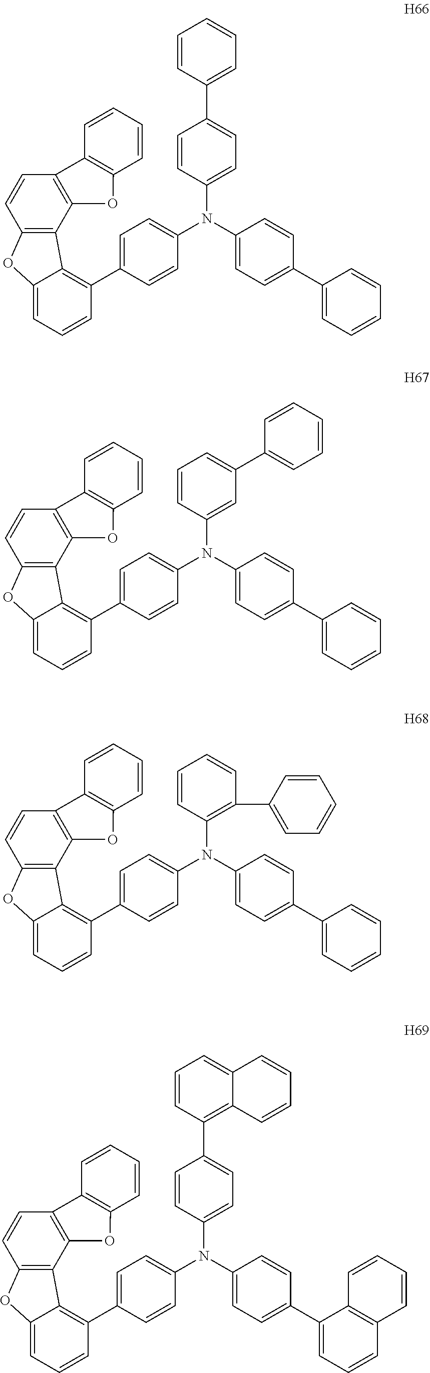 [SMILES: c1ccc(-c2ccc(N(c3ccc(-c4ccccc4)cc3)c3ccc(-c4cccc5oc6ccc7c8ccccc8oc7c6c45)cc3)cc2)cc1 ; c1ccc(-c2ccc(N(c3ccc(-c4cccc5oc6ccc7c8ccccc8oc7c6c45)cc3)c3cccc(-c4ccccc4)c3)cc2)cc1 ; c1ccc(-c2ccc(N(c3ccc(-c4cccc5oc6ccc7c8ccccc8oc7c6c45)cc3)c3ccccc3-c3ccccc3)cc2)cc1 ; c1ccc2c(-c3ccc(N(c4ccc(-c5cccc6ccccc56)cc4)c4ccc(-c5cccc6oc7ccc8c9ccccc9oc8c7c56)cc4)cc3)cccc2c1]

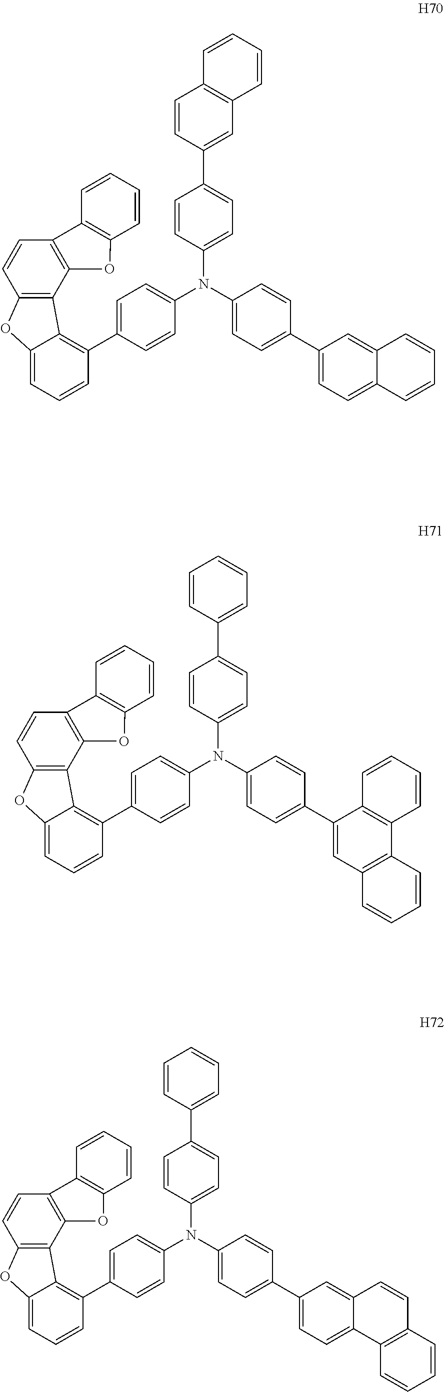 [SMILES: c1ccc2cc(-c3ccc(N(c4ccc(-c5ccc6ccccc6c5)cc4)c4ccc(-c5cccc6oc7ccc8c9ccccc9oc8c7c56)cc4)cc3)ccc2c1 ; c1ccc(-c2ccc(N(c3ccc(-c4cc5ccccc5c5ccccc45)cc3)c3ccc(-c4cccc5oc6ccc7c8ccccc8oc7c6c45)cc3)cc2)cc1 ; c1ccc(-c2ccc(N(c3ccc(-c4ccc5c(ccc6ccccc65)c4)cc3)c3ccc(-c4cccc5oc6ccc7c8ccccc8oc7c6c45)cc3)cc2)cc1]

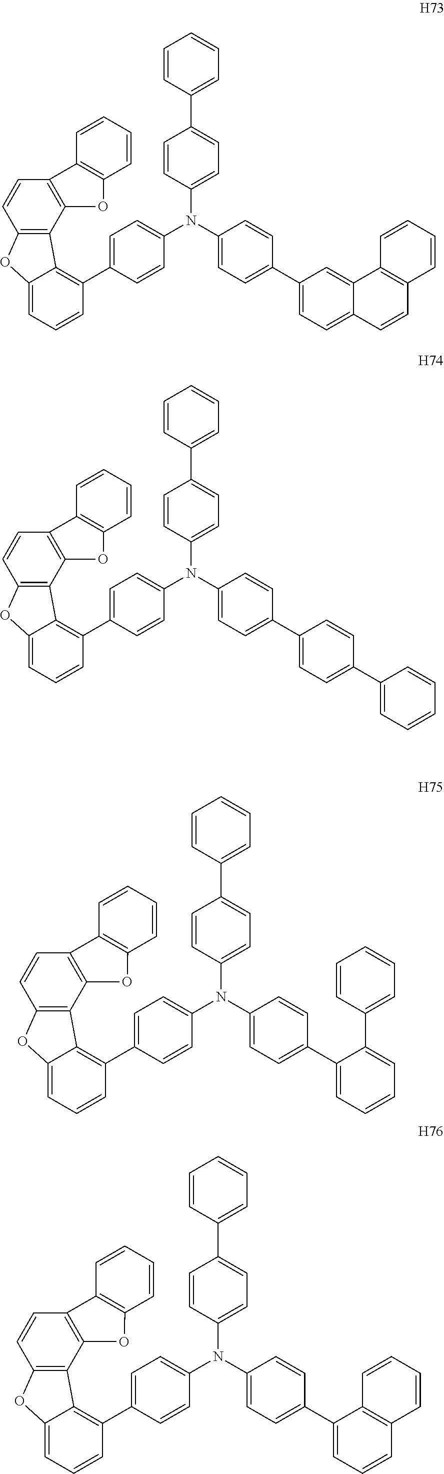 [SMILES: c1ccc(-c2ccc(N(c3ccc(-c4ccc5ccc6ccccc6c5c4)cc3)c3ccc(-c4cccc5oc6ccc7c8ccccc8oc7c6c45)cc3)cc2)cc1 ; c1ccc(-c2ccc(-c3ccc(N(c4ccc(-c5ccccc5)cc4)c4ccc(-c5cccc6oc7ccc8c9ccccc9oc8c7c56)cc4)cc3)cc2)cc1 ; c1ccc(-c2ccc(N(c3ccc(-c4ccccc4-c4ccccc4)cc3)c3ccc(-c4cccc5oc6ccc7c8ccccc8oc7c6c45)cc3)cc2)cc1 ; c1ccc(-c2ccc(N(c3ccc(-c4cccc5ccccc45)cc3)c3ccc(-c4cccc5oc6ccc7c8ccccc8oc7c6c45)cc3)cc2)cc1]

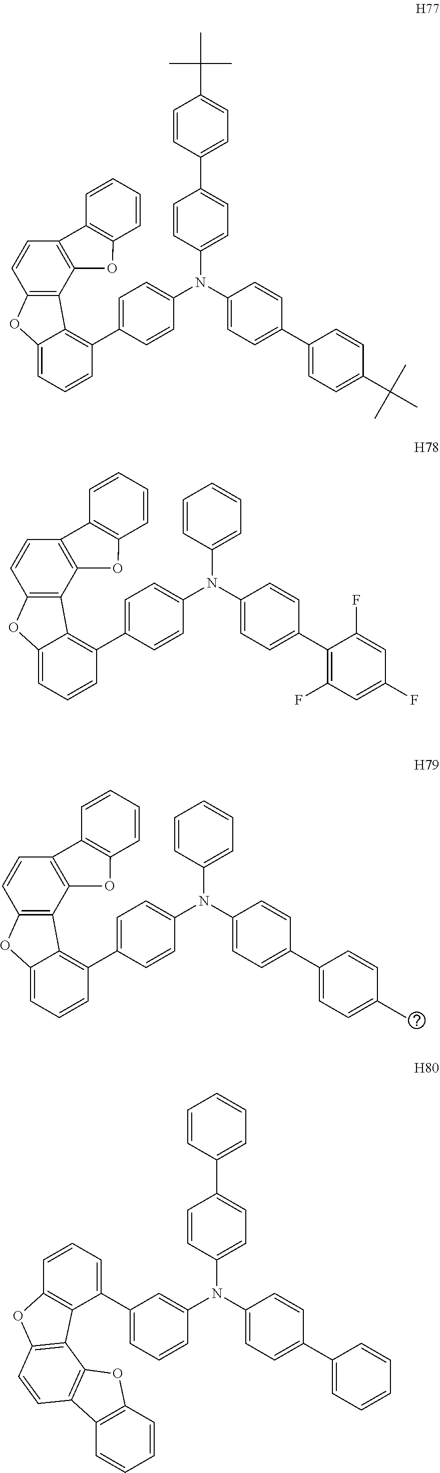 [SMILES: CC(C)(C)c1ccc(-c2ccc(N(c3ccc(-c4ccc(C(C)(C)C)cc4)cc3)c3ccc(-c4cccc5oc6ccc7c8ccccc8oc7c6c45)cc3)cc2)cc1 ; Fc1cc(F)c(-c2ccc(N(c3ccccc3)c3ccc(-c4cccc5oc6ccc7c8ccccc8oc7c6c45)cc3)cc2)c(F)c1 ; Cc1ccc(-c2ccc(N(c3ccccc3)c3ccc(-c4cccc5oc6ccc7c8ccccc8oc7c6c45)cc3)cc2)cc1 ; c1ccc(-c2ccc(N(c3ccc(-c4ccccc4)cc3)c3cccc(-c4cccc5oc6ccc7c8ccccc8oc7c6c45)c3)cc2)cc1]

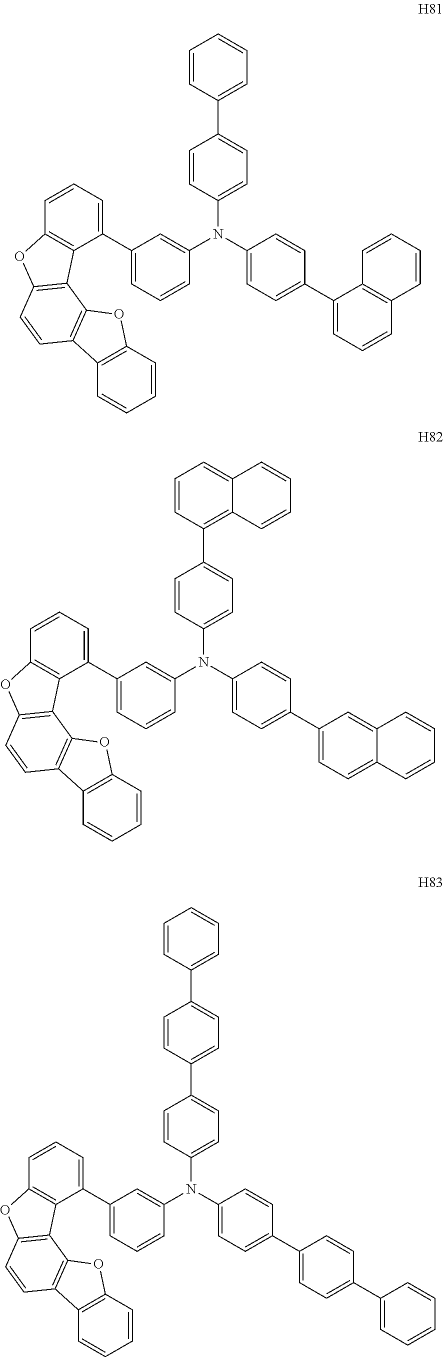 [SMILES: c1ccc(-c2ccc(N(c3ccc(-c4cccc5ccccc45)cc3)c3cccc(-c4cccc5oc6ccc7c8ccccc8oc7c6c45)c3)cc2)cc1 ; c1cc(-c2cccc3oc4ccc5c6ccccc6oc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6oc7ccc8c9ccccc9oc8c7c56)c4)cc3)cc2)cc1]

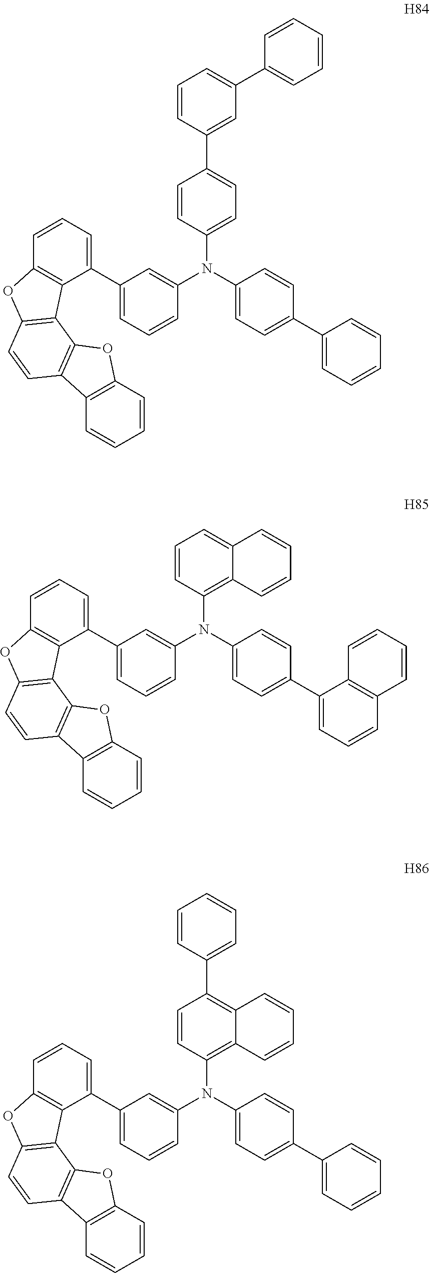 [SMILES: c1ccc(-c2ccc(N(c3ccc(-c4cccc(-c5ccccc5)c4)cc3)c3cccc(-c4cccc5oc6ccc7c8ccccc8oc7c6c45)c3)cc2)cc1 ; c1cc(-c2cccc3oc4ccc5c6ccccc6oc5c4c23)cc(N(c2ccc(-c3cccc4ccccc34)cc2)c2cccc3ccccc23)c1 ; c1ccc(-c2ccc(N(c3cccc(-c4cccc5oc6ccc7c8ccccc8oc7c6c45)c3)c3ccc(-c4ccccc4)c4ccccc34)cc2)cc1]

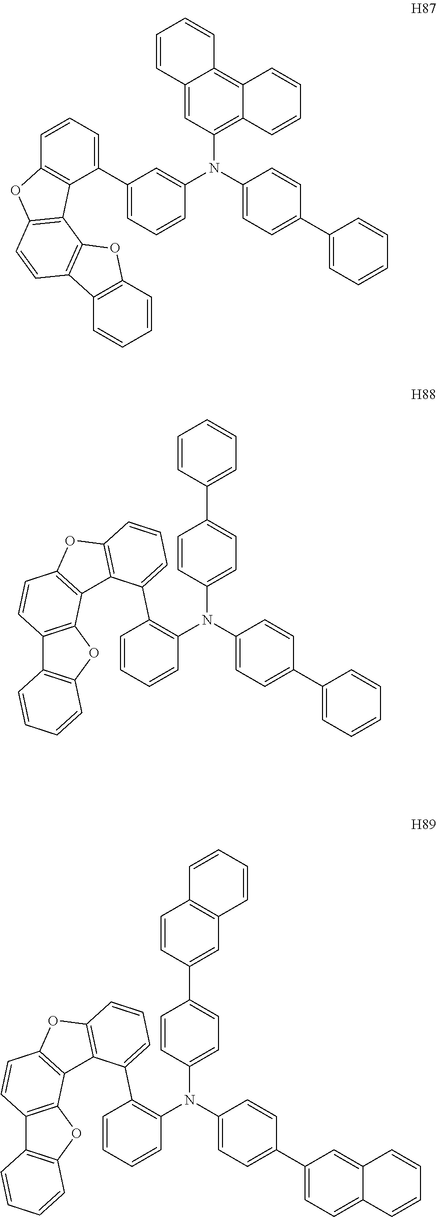 [SMILES: c1ccc(-c2ccc(N(c3cccc(-c4cccc5oc6ccc7c8ccccc8oc7c6c45)c3)c3cc4ccccc4c4ccccc34)cc2)cc1 ; c1ccc(-c2ccc(N(c3ccc(-c4ccccc4)cc3)c3ccccc3-c3cccc4oc5ccc6c7ccccc7oc6c5c34)cc2)cc1 ; c1ccc(N(c2ccc(-c3ccc4ccccc4c3)cc2)c2ccc(-c3ccc4ccccc4c3)cc2)c(-c2cccc3oc4ccc5c6ccccc6oc5c4c23)c1]

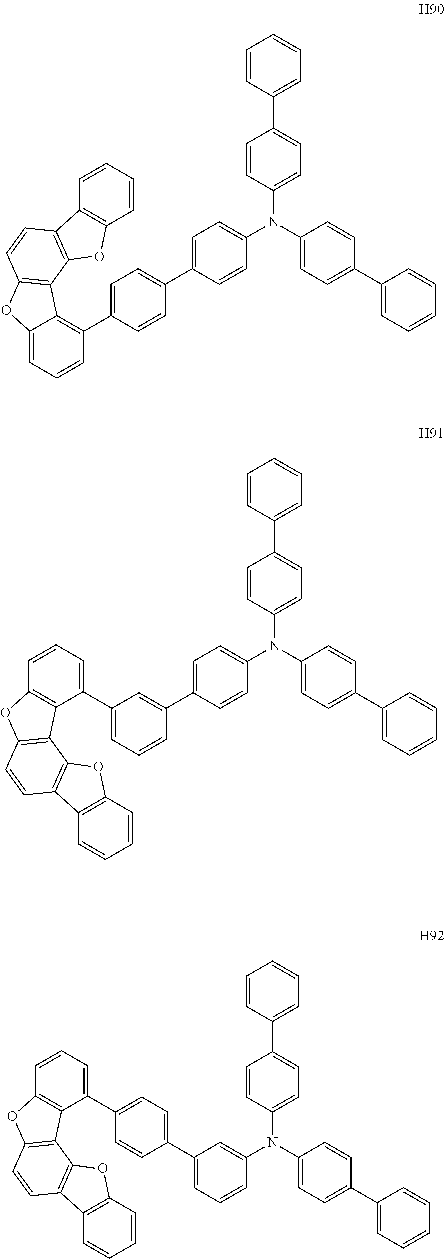 [SMILES: c1ccc(-c2ccc(N(c3ccc(-c4ccccc4)cc3)c3ccc(-c4ccc(-c5cccc6oc7ccc8c9ccccc9oc8c7c56)cc4)cc3)cc2)cc1 ; c1ccc(-c2ccc(N(c3ccc(-c4ccccc4)cc3)c3ccc(-c4cccc(-c5cccc6oc7ccc8c9ccccc9oc8c7c56)c4)cc3)cc2)cc1 ; c1ccc(-c2ccc(N(c3ccc(-c4ccccc4)cc3)c3cccc(-c4ccc(-c5cccc6oc7ccc8c9ccccc9oc8c7c56)cc4)c3)cc2)cc1]

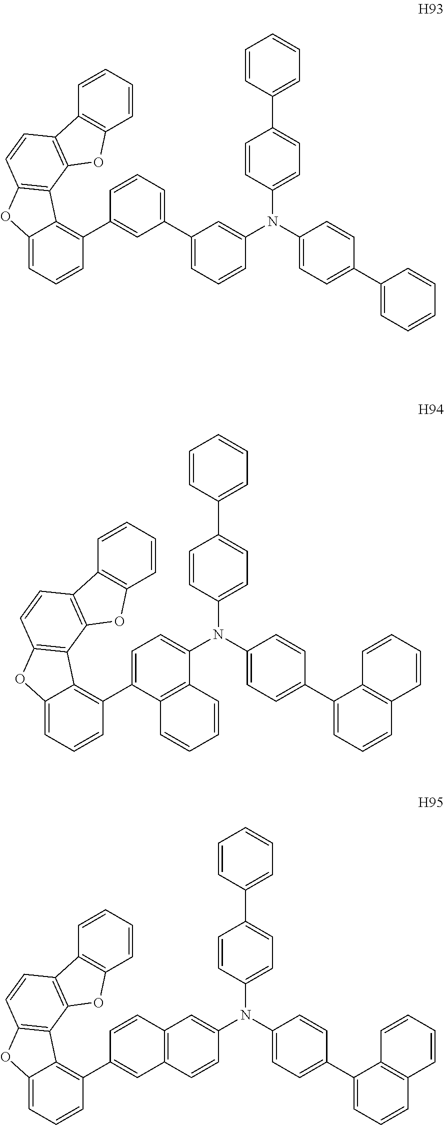 [SMILES: c1ccc(-c2ccc(N(c3ccc(-c4ccccc4)cc3)c3cccc(-c4cccc(-c5cccc6oc7ccc8c9ccccc9oc8c7c56)c4)c3)cc2)cc1 ; c1ccc(-c2ccc(N(c3ccc(-c4cccc5ccccc45)cc3)c3ccc(-c4cccc5oc6ccc7c8ccccc8oc7c6c45)c4ccccc34)cc2)cc1 ; c1ccc(-c2ccc(N(c3ccc(-c4cccc5ccccc45)cc3)c3ccc4cc(-c5cccc6oc7ccc8c9ccccc9oc8c7c56)ccc4c3)cc2)cc1]

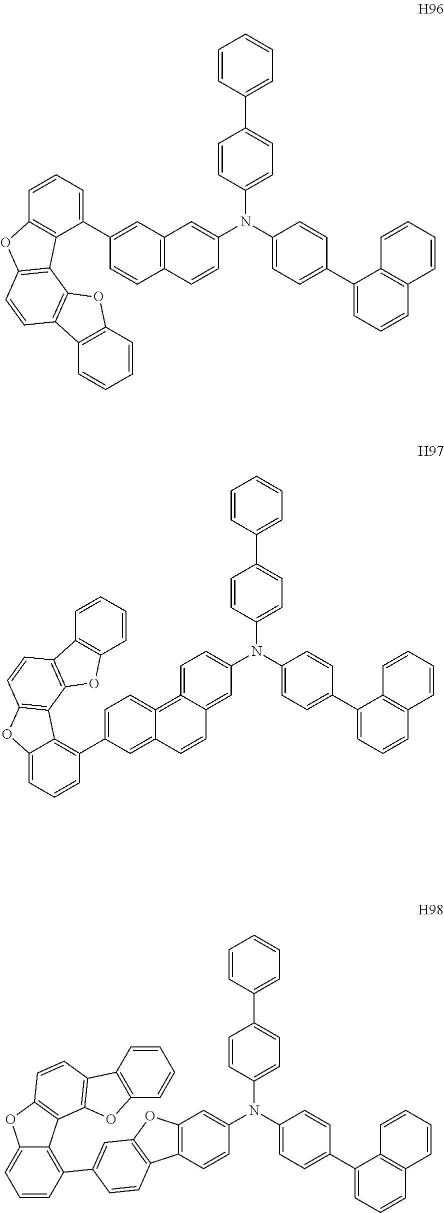

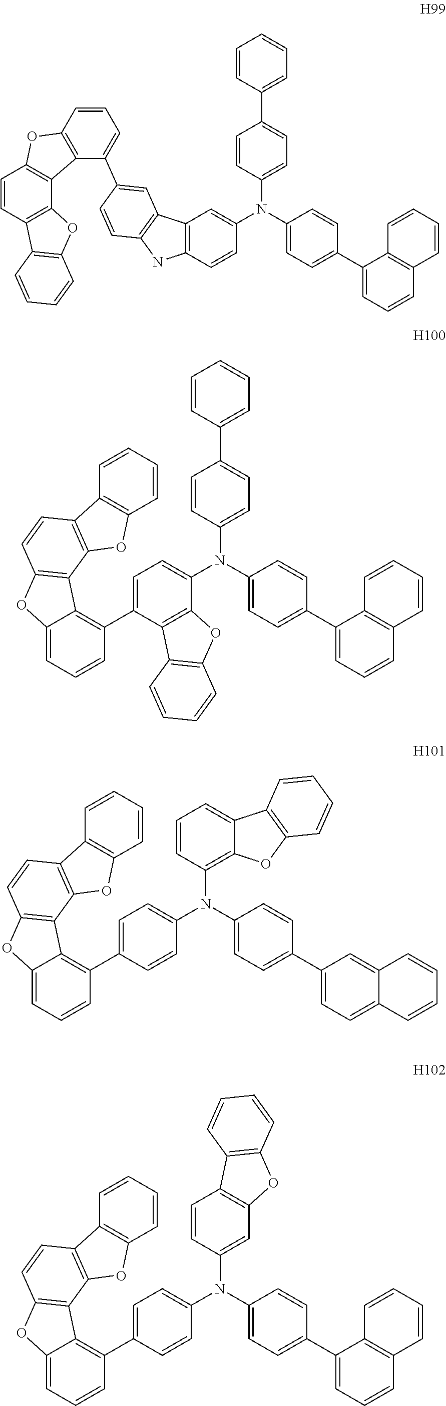 [SMILES: c1ccc(-c2ccc(N(c3ccc(-c4cccc5ccccc45)cc3)c3ccc4[nH]c5ccc(-c6cccc7oc8ccc9c%10ccccc%10oc9c8c67)cc5c4c3)cc2)cc1 ; c1ccc(-c2ccc(N(c3ccc(-c4cccc5ccccc45)cc3)c3ccc(-c4cccc5oc6ccc7c8ccccc8oc7c6c45)c4c3oc3ccccc34)cc2)cc1 ; c1ccc2cc(-c3ccc(N(c4ccc(-c5cccc6oc7ccc8c9ccccc9oc8c7c56)cc4)c4cccc5c4oc4ccccc45)cc3)ccc2c1 ; c1ccc2c(-c3ccc(N(c4ccc(-c5cccc6oc7ccc8c9ccccc9oc8c7c56)cc4)c4ccc5c(c4)oc4ccccc45)cc3)cccc2c1]

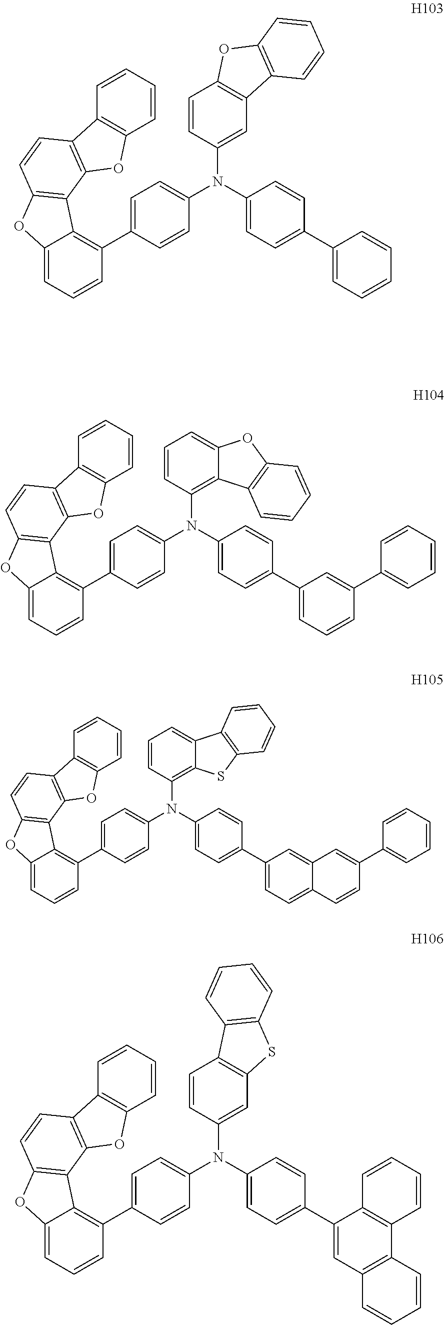 [SMILES: c1ccc(-c2ccc(N(c3ccc(-c4cccc5oc6ccc7c8ccccc8oc7c6c45)cc3)c3ccc4oc5ccccc5c4c3)cc2)cc1 ; c1ccc(-c2cccc(-c3ccc(N(c4ccc(-c5cccc6oc7ccc8c9ccccc9oc8c7c56)cc4)c4cccc5oc6ccccc6c45)cc3)c2)cc1 ; c1ccc(-c2ccc3ccc(-c4ccc(N(c5ccc(-c6cccc7oc8ccc9c%10ccccc%10oc9c8c67)cc5)c5cccc6c5sc5ccccc56)cc4)cc3c2)cc1 ; c1ccc2c(c1)cc(-c1ccc(N(c3ccc(-c4cccc5oc6ccc7c8ccccc8oc7c6c45)cc3)c3ccc4c(c3)sc3ccccc34)cc1)c1ccccc12]

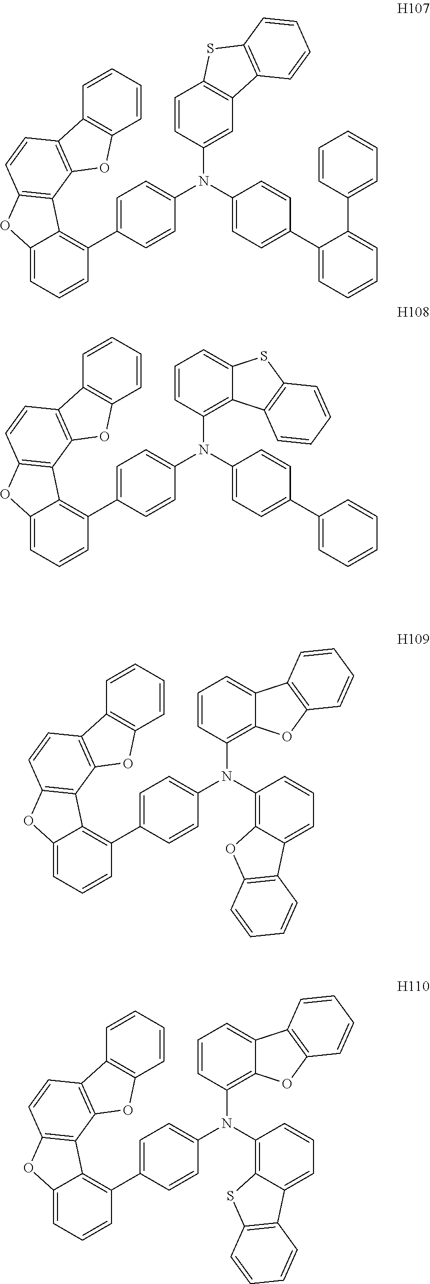 [SMILES: c1ccc(-c2ccccc2-c2ccc(N(c3ccc(-c4cccc5oc6ccc7c8ccccc8oc7c6c45)cc3)c3ccc4sc5ccccc5c4c3)cc2)cc1 ; c1ccc(-c2ccc(N(c3ccc(-c4cccc5oc6ccc7c8ccccc8oc7c6c45)cc3)c3cccc4sc5ccccc5c34)cc2)cc1 ; c1ccc2c(c1)oc1c(N(c3ccc(-c4cccc5oc6ccc7c8ccccc8oc7c6c45)cc3)c3cccc4c3oc3ccccc34)cccc12 ; c1ccc2c(c1)oc1c(N(c3ccc(-c4cccc5oc6ccc7c8ccccc8oc7c6c45)cc3)c3cccc4c3sc3ccccc34)cccc12]

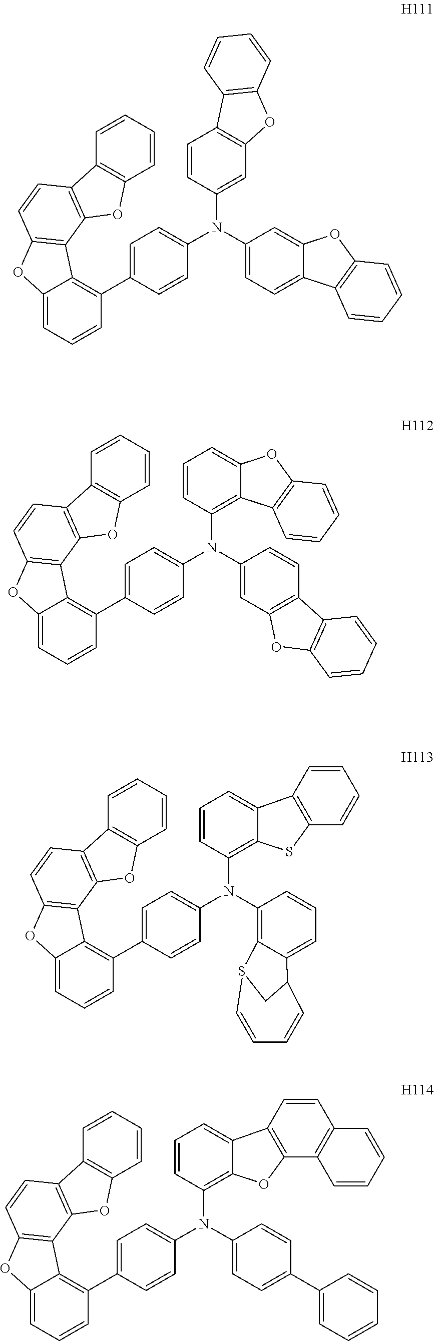

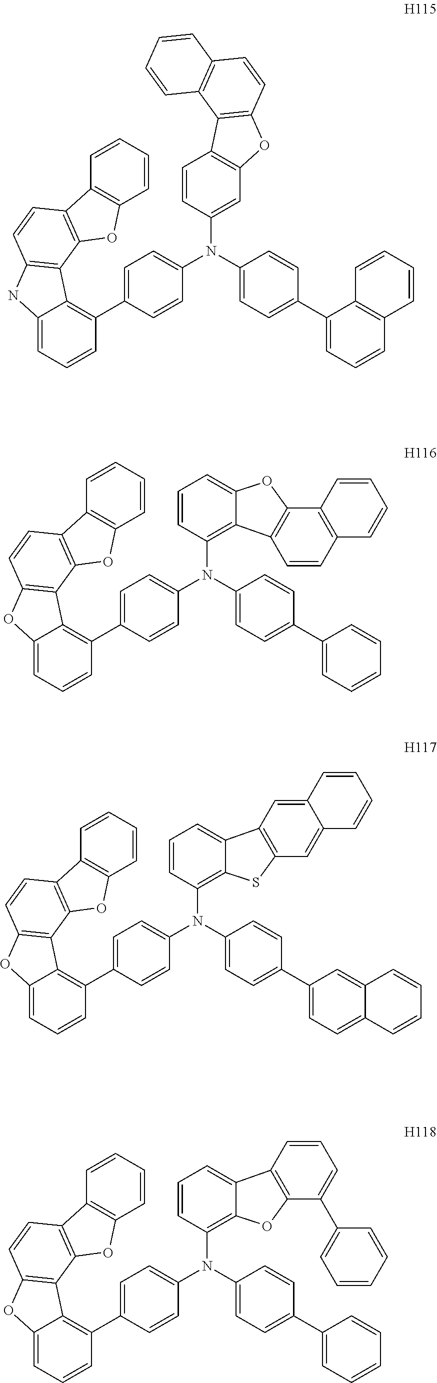 [SMILES: c1ccc2c(-c3ccc(N(c4ccc(-c5cccc6[nH]c7ccc8c9ccccc9oc8c7c56)cc4)c4ccc5c(c4)oc4ccc6ccccc6c45)cc3)cccc2c1 ; c1ccc(-c2ccc(N(c3ccc(-c4cccc5oc6ccc7c8ccccc8oc7c6c45)cc3)c3cccc4oc5c6ccccc6ccc5c34)cc2)cc1 ; c1ccc2cc(-c3ccc(N(c4ccc(-c5cccc6oc7ccc8c9ccccc9oc8c7c56)cc4)c4cccc5c4sc4cc6ccccc6cc45)cc3)ccc2c1 ; c1ccc(-c2ccc(N(c3ccc(-c4cccc5oc6ccc7c8ccccc8oc7c6c45)cc3)c3cccc4c3oc3c(-c5ccccc5)cccc34)cc2)cc1]

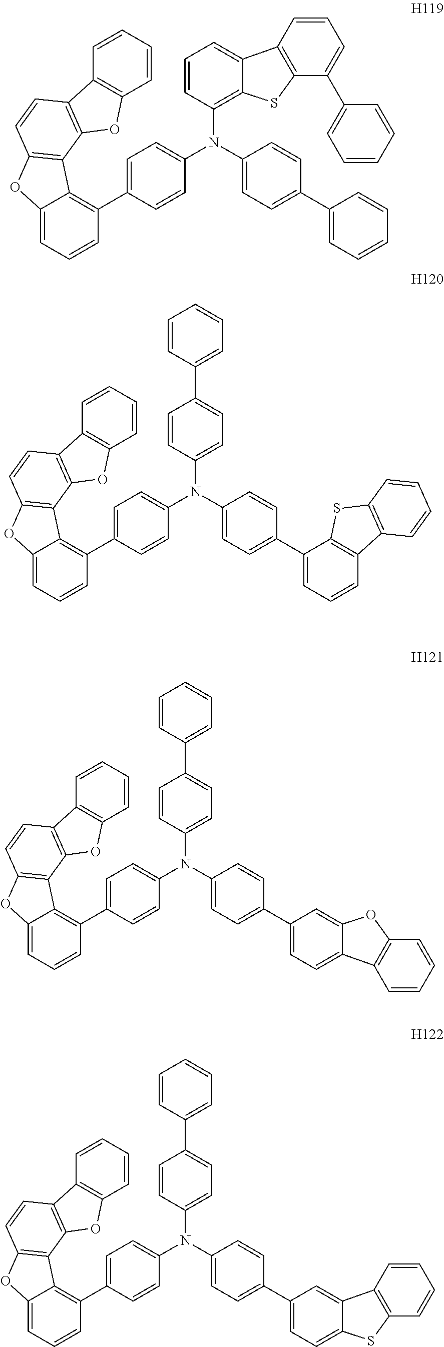 [SMILES: c1ccc(-c2ccc(N(c3ccc(-c4cccc5oc6ccc7c8ccccc8oc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5oc6ccc7c8ccccc8oc7c6c45)cc3)cc2)cc1 ; c1ccc(-c2ccc(N(c3ccc(-c4ccc5c(c4)oc4ccccc45)cc3)c3ccc(-c4cccc5oc6ccc7c8ccccc8oc7c6c45)cc3)cc2)cc1 ; c1ccc(-c2ccc(N(c3ccc(-c4ccc5sc6ccccc6c5c4)cc3)c3ccc(-c4cccc5oc6ccc7c8ccccc8oc7c6c45)cc3)cc2)cc1]

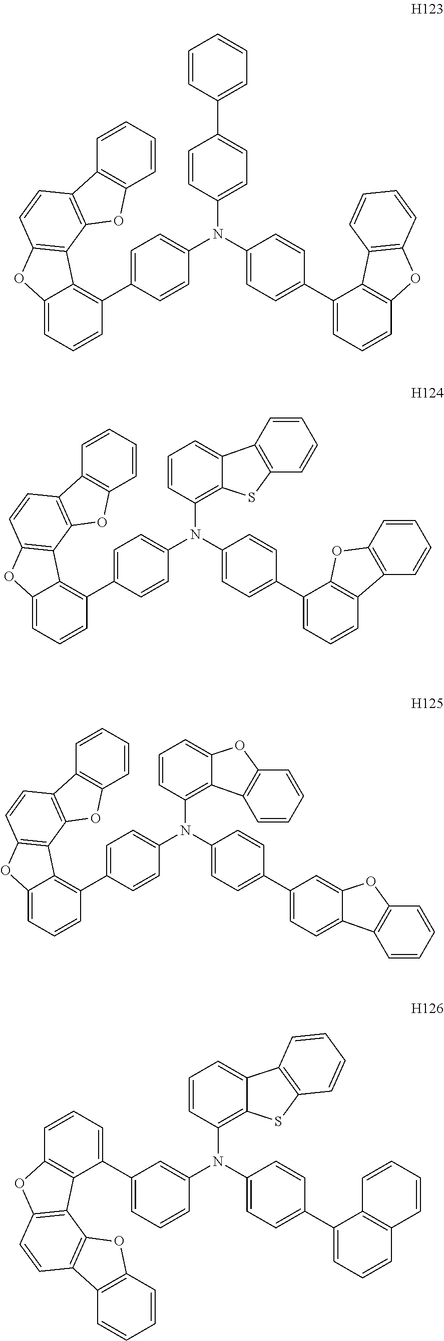 [SMILES: c1ccc(-c2ccc(N(c3ccc(-c4cccc5oc6ccccc6c45)cc3)c3ccc(-c4cccc5oc6ccc7c8ccccc8oc7c6c45)cc3)cc2)cc1 ; c1ccc2c(c1)oc1c(-c3ccc(N(c4ccc(-c5cccc6oc7ccc8c9ccccc9oc8c7c56)cc4)c4cccc5c4sc4ccccc45)cc3)cccc12 ; c1ccc2c(c1)oc1cc(-c3ccc(N(c4ccc(-c5cccc6oc7ccc8c9ccccc9oc8c7c56)cc4)c4cccc5oc6ccccc6c45)cc3)ccc12 ; c1cc(-c2cccc3oc4ccc5c6ccccc6oc5c4c23)cc(N(c2ccc(-c3cccc4ccccc34)cc2)c2cccc3c2sc2ccccc23)c1]

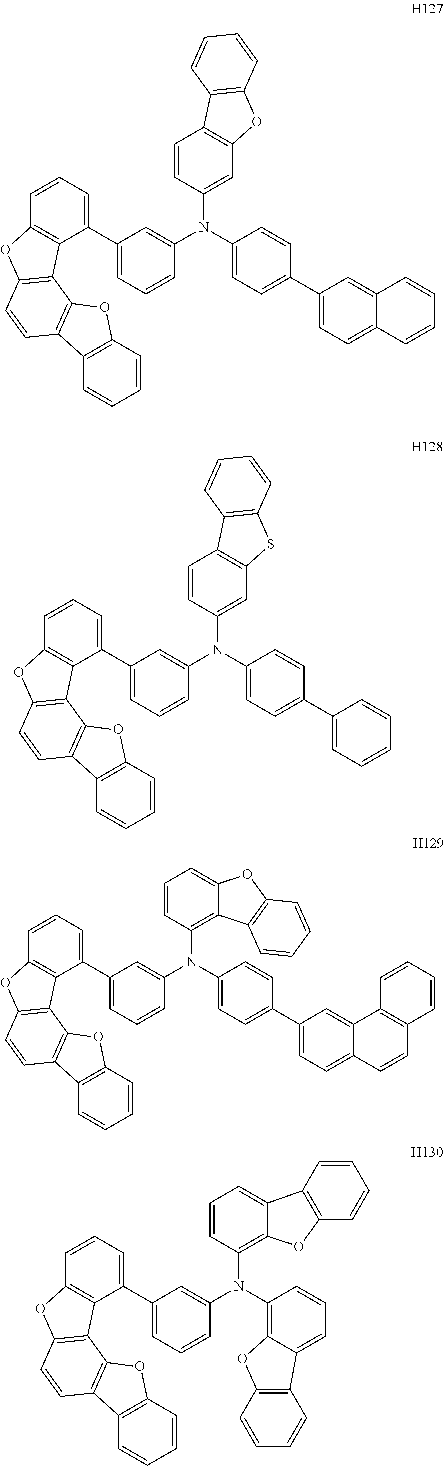 [SMILES: c1cc(-c2cccc3oc4ccc5c6ccccc6oc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5oc6ccc7c8ccccc8oc7c6c45)c3)c3ccc4c(c3)sc3ccccc34)cc2)cc1 ; c1cc(-c2cccc3oc4ccc5c6ccccc6oc5c4c23)cc(N(c2ccc(-c3ccc4ccc5ccccc5c4c3)cc2)c2cccc3oc4ccccc4c23)c1 ; c1cc(-c2cccc3oc4ccc5c6ccccc6oc5c4c23)cc(N(c2cccc3c2oc2ccccc23)c2cccc3c2oc2ccccc23)c1]

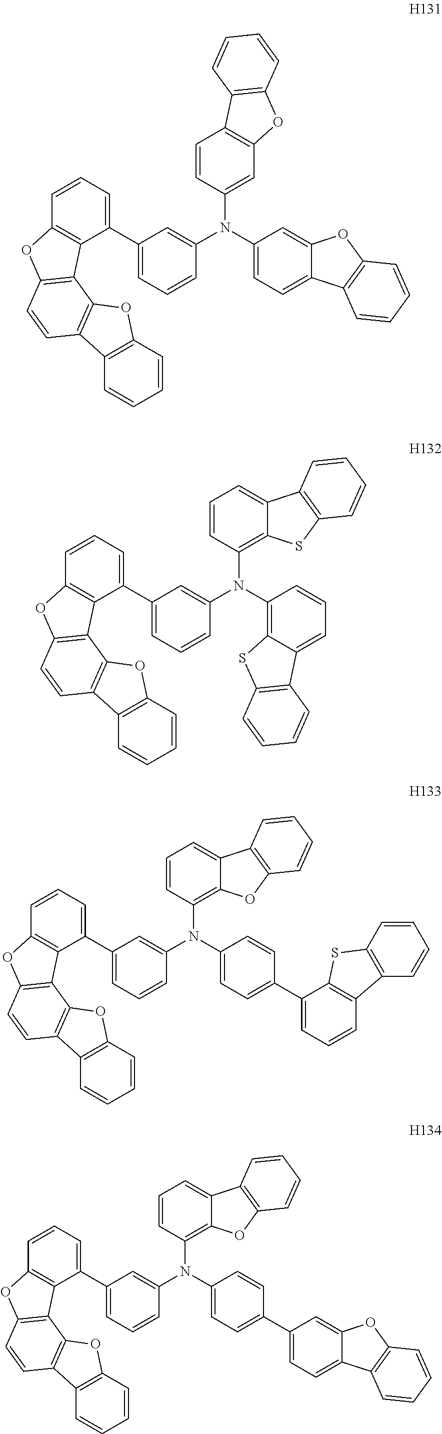 [SMILES: c1cc(-c2cccc3oc4ccc5c6ccccc6oc5c4c23)cc(N(c2ccc3c(c2)oc2ccccc23)c2ccc3c(c2)oc2ccccc23)c1 ; c1cc(-c2cccc3oc4ccc5c6ccccc6oc5c4c23)cc(N(c2cccc3c2sc2ccccc23)c2cccc3c2sc2ccccc23)c1 ; c1cc(-c2cccc3oc4ccc5c6ccccc6oc5c4c23)cc(N(c2ccc(-c3cccc4c3sc3ccccc34)cc2)c2cccc3c2oc2ccccc23)c1 ; c1cc(-c2cccc3oc4ccc5c6ccccc6oc5c4c23)cc(N(c2ccc(-c3ccc4c(c3)oc3ccccc34)cc2)c2cccc3c2oc2ccccc23)c1]

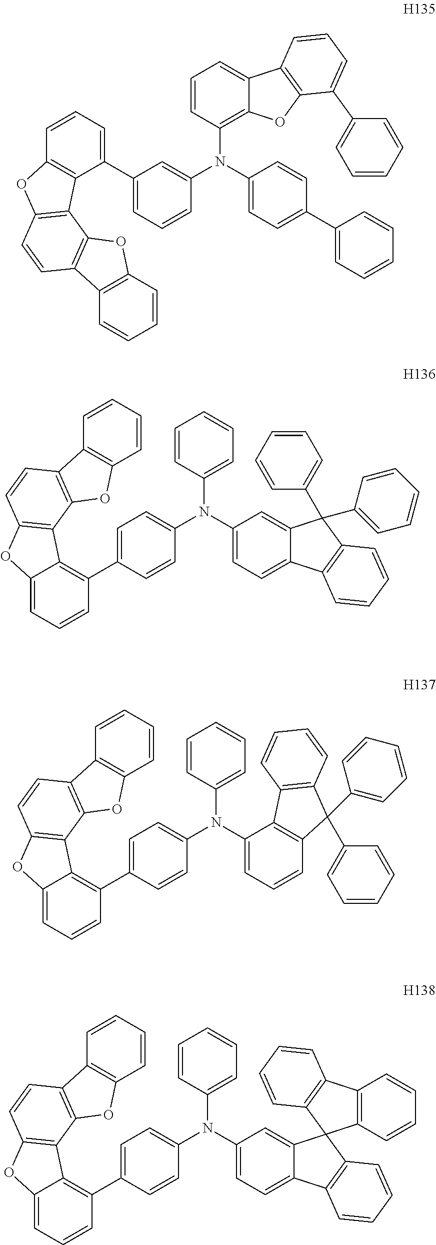 [SMILES: c1ccc(-c2ccc(N(c3cccc(-c4cccc5oc6ccc7c8ccccc8oc7c6c45)c3)c3cccc4c3oc3c(-c5ccccc5)cccc34)cc2)cc1 ; c1ccc(N(c2ccc(-c3cccc4oc5ccc6c7ccccc7oc6c5c34)cc2)c2ccc3c(c2)C(c2ccccc2)(c2ccccc2)c2ccccc2-3)cc1 ; c1ccc(N(c2ccc(-c3cccc4oc5ccc6c7ccccc7oc6c5c34)cc2)c2cccc3c2-c2ccccc2C3(c2ccccc2)c2ccccc2)cc1 ; c1ccc(N(c2ccc(-c3cccc4oc5ccc6c7ccccc7oc6c5c34)cc2)c2ccc3c(c2)C2(c4ccccc4-c4ccccc42)c2ccccc2-3)cc1]

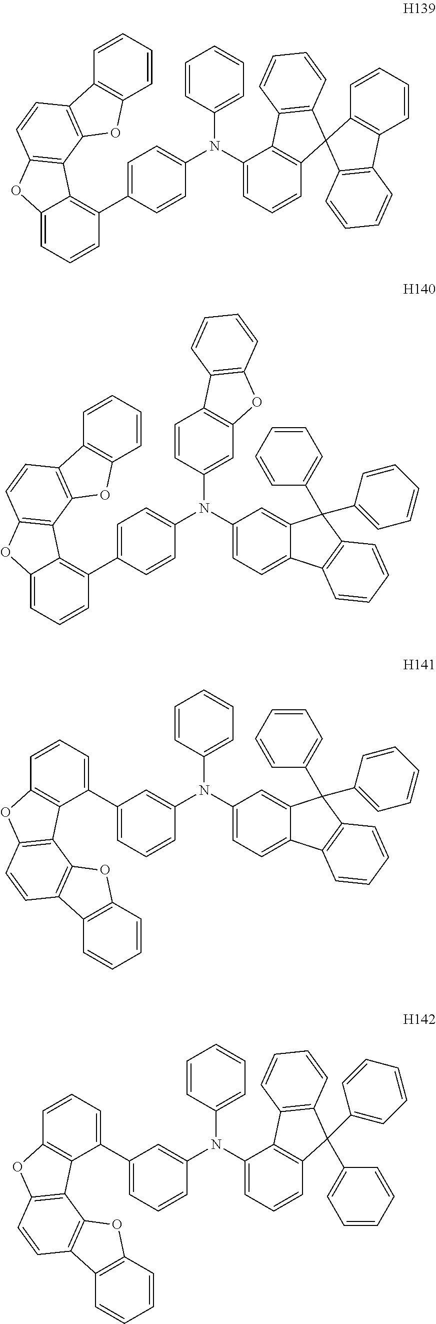 [SMILES: c1ccc(N(c2ccc(-c3cccc4oc5ccc6c7ccccc7oc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6oc7ccc8c9ccccc9oc8c7c56)cc4)c4ccc5c(c4)oc4ccccc45)cc32)cc1 ; c1ccc(N(c2cccc(-c3cccc4oc5ccc6c7ccccc7oc6c5c34)c2)c2ccc3c(c2)C(c2ccccc2)(c2ccccc2)c2ccccc2-3)cc1 ; c1ccc(N(c2cccc(-c3cccc4oc5ccc6c7ccccc7oc6c5c34)c2)c2cccc3c2-c2ccccc2C3(c2ccccc2)c2ccccc2)cc1]

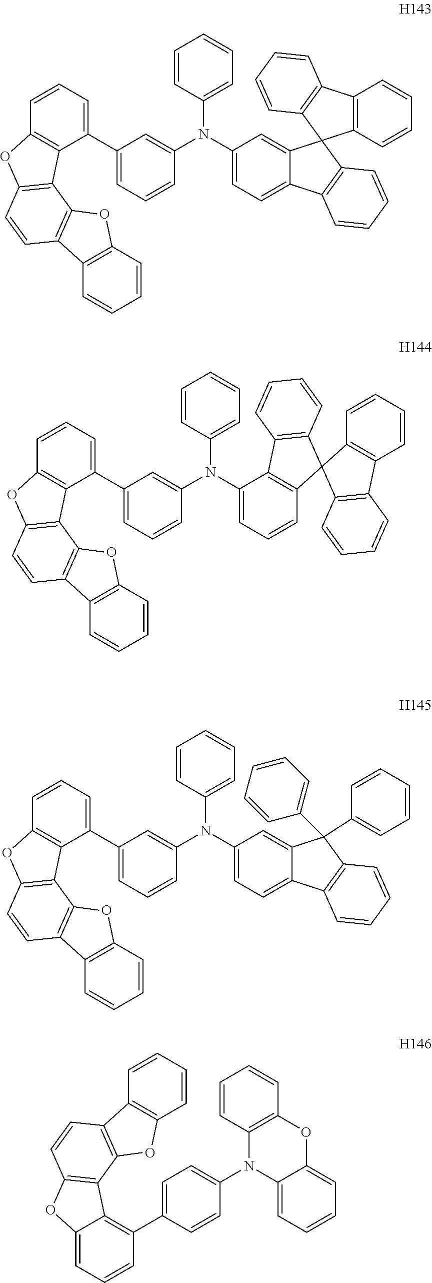 [SMILES: c1ccc(N(c2cccc(-c3cccc4oc5ccc6c7ccccc7oc6c5c34)c2)c2ccc3c(c2)C2(c4ccccc4-c4ccccc42)c2ccccc2-3)cc1 ; c1ccc(N(c2cccc(-c3cccc4oc5ccc6c7ccccc7oc6c5c34)c2)c2cccc3c2-c2ccccc2C32c3ccccc3-c3ccccc32)cc1 ; c1ccc(N(c2cccc(-c3cccc4oc5ccc6c7ccccc7oc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3oc4ccc5c6ccccc6oc5c4c23)cc1]

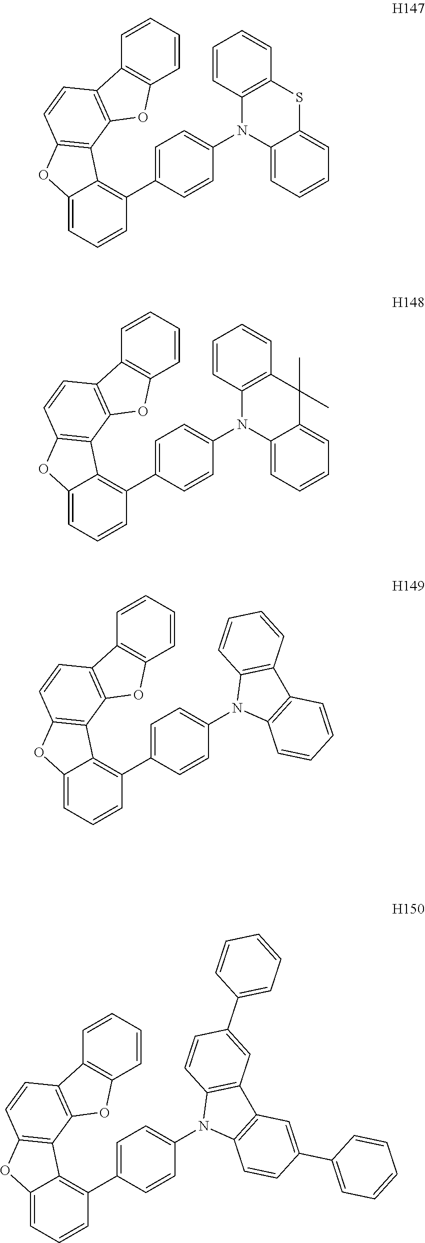 [SMILES: c1ccc2c(c1)Sc1ccccc1N2c1ccc(-c2cccc3oc4ccc5c6ccccc6oc5c4c23)cc1 ; CC1(C)c2ccccc2N(c2ccc(-c3cccc4oc5ccc6c7ccccc7oc6c5c34)cc2)c2ccccc21 ; c1ccc2c(c1)oc1c2ccc2oc3cccc(-c4ccc(-n5c6ccccc6c6ccccc65)cc4)c3c21 ; c1ccc(-c2ccc3c(c2)c2cc(-c4ccccc4)ccc2n3-c2ccc(-c3cccc4oc5ccc6c7ccccc7oc6c5c34)cc2)cc1]

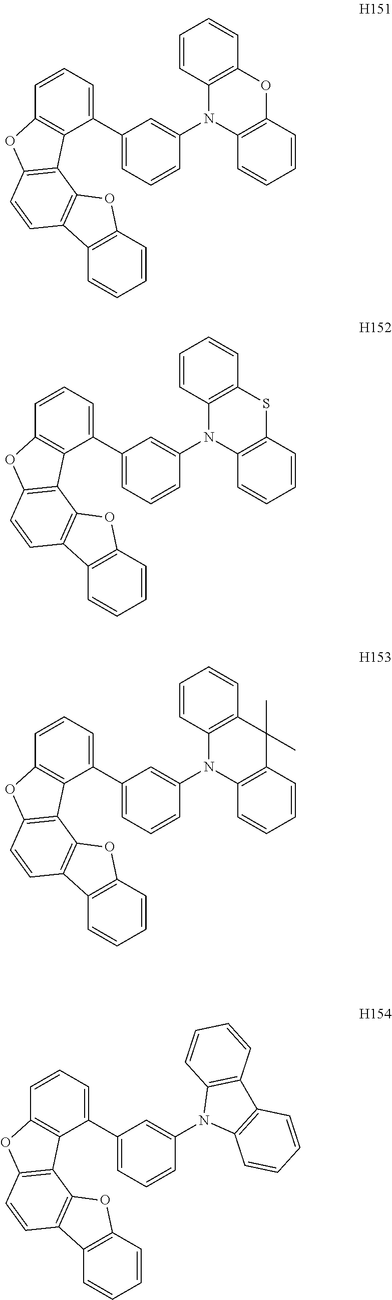 [SMILES: c1cc(-c2cccc3oc4ccc5c6ccccc6oc5c4c23)cc(N2c3ccccc3Oc3ccccc32)c1 ; c1cc(-c2cccc3oc4ccc5c6ccccc6oc5c4c23)cc(N2c3ccccc3Sc3ccccc32)c1 ; CC1(C)c2ccccc2N(c2cccc(-c3cccc4oc5ccc6c7ccccc7oc6c5c34)c2)c2ccccc21 ; c1cc(-c2cccc3oc4ccc5c6ccccc6oc5c4c23)cc(-n2c3ccccc3c3ccccc32)c1]

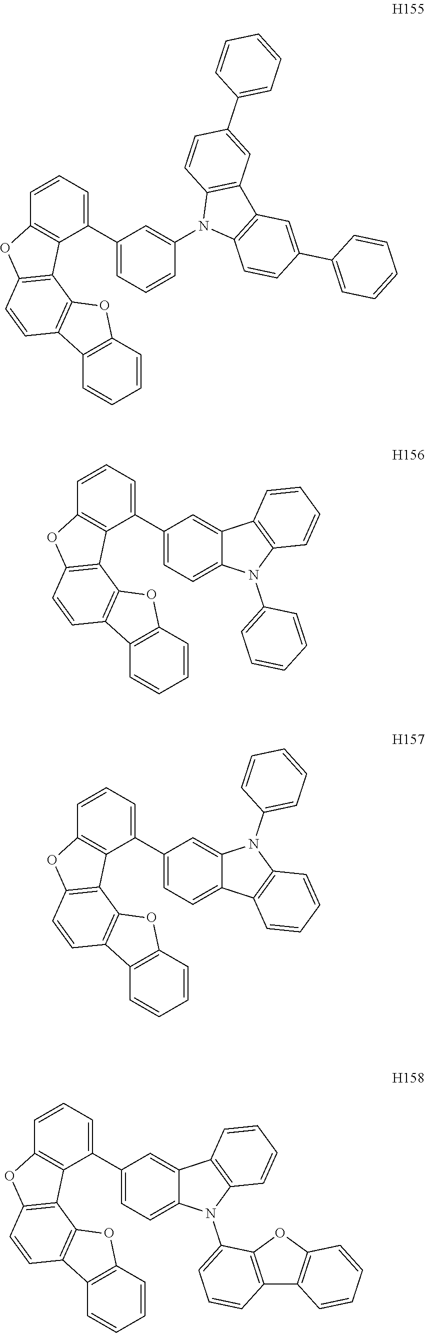 [SMILES: c1ccc(-c2ccc3c(c2)c2cc(-c4ccccc4)ccc2n3-c2cccc(-c3cccc4oc5ccc6c7ccccc7oc6c5c34)c2)cc1 ; c1ccc(-n2c3ccccc3c3cc(-c4cccc5oc6ccc7c8ccccc8oc7c6c45)ccc32)cc1 ; c1ccc(-n2c3ccccc3c3ccc(-c4cccc5oc6ccc7c8ccccc8oc7c6c45)cc32)cc1 ; c1ccc2c(c1)oc1c(-n3c4ccccc4c4cc(-c5cccc6oc7ccc8c9ccccc9oc8c7c56)ccc43)cccc12]

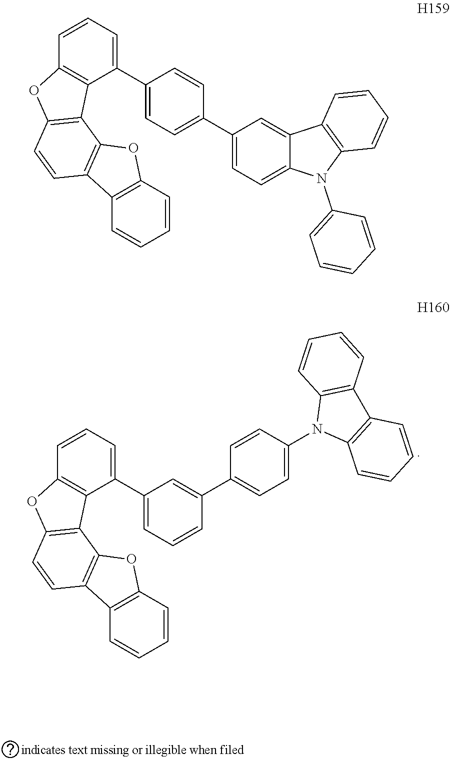 [SMILES: c1ccc(-n2c3ccccc3c3cc(-c4ccc(-c5cccc6oc7ccc8c9ccccc9oc8c7c56)cc4)ccc32)cc1 ; c1cc(-c2ccc(-n3c4ccccc4c4ccccc43)cc2)cc(-c2cccc3oc4ccc5c6ccccc6oc5c4c23)c1]

13. A polycyclic compound represented by Formula 1: 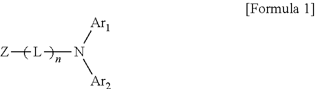
wherein in Formula 1
n is an integer from 0 to 3,
L is a substituted or unsubstituted arylene group having 6 to 40 ring-forming carbon atoms and excluding fluorene, or a substituted or unsubstituted heteroarylene group having 2 to 40 ring-forming carbon atoms and excluding N as a ring-forming atom, or is bonded to Ar₁ or Ar₂ by using a single bond, O, S, or C(R₁)(R₂) as a linker to form a ring,
Ar₁ and Ar₂ are each independently a substituted or unsubstituted aryl group having 6 to 40 ring-forming carbon atoms and excluding triphenylene, or a substituted or unsubstituted heteroaryl group having 2 to 40 ring-forming carbon atoms, or are bonded to L or an adjacent substituent by using a single bond, O, S, or C(R₁)(R₂) as a linker to form a ring, and
R₁ and R₂ are each independently a substituted or unsubstituted alkyl group having 1 to 20 carbon atoms, or a substituted or unsubstituted aryl group having 6 to 40 ring-forming carbon atoms, and
Z is a group represented by Formula 2-1 or Formula 2-2: 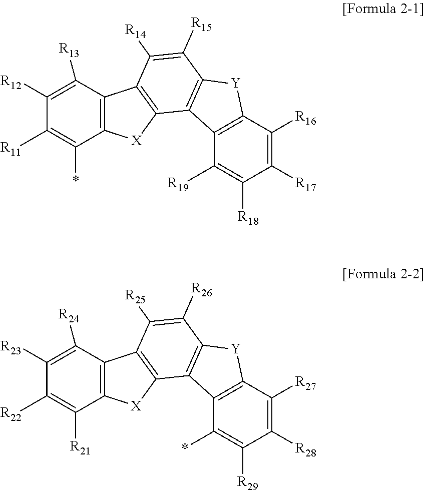
wherein in Formula 2-1 and Formula 2-2,
X and Y are each independently O or S,
R₁₁ to R₁₉ and R₂₁ to R₂₉ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, or are bonded to an adjacent group to form a ring, and
* indicates a binding site to a neighboring atom.

14. The polycyclic compound of claim 13, wherein Formula 1 is represented by one of Formula 1-1 to Formula 1-4: 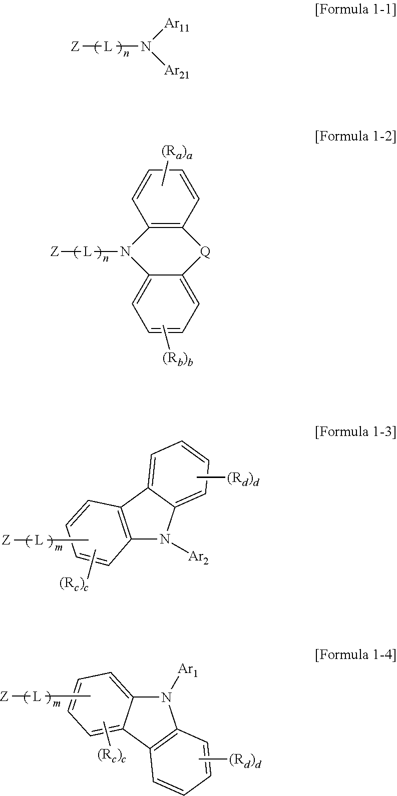
wherein in Formula 1-1,
Ar₁₁ and Ar₂₁ are each independently a substituted or unsubstituted aryl group having 6 to 40 ring-forming carbon atoms and excluding triphenylene, or a substituted or unsubstituted heteroaryl group having 2 to 40 ring-forming carbon atoms and excluding N as a ring-forming atom,
in Formula 1-2,
Q is a single bond, O, S, or C(R₁)(R₂),
a and b are each independently an integer from 0 to 4,
in Formula 1-3 and Formula 1-4,
m is an integer from 0 to 2,
c is an integer from 0 to 3,
d is an integer from 0 to 4, and
in Formula 1-2 to 1-4,
Rₐ to R_{d} are each independently a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms, or bonded to an adjacent group to form an aromatic ring, and
in Formula 1-1 to Formula 1-4,
Z, L, n, R₁, R₂, Ar₁, and Ar₂ are the same as defined in connection with Formula 1.

15. The polycyclic compound of claim 13, wherein Formula 2-1 is represented by one of Formula 2-1A to Formula 2-1D: 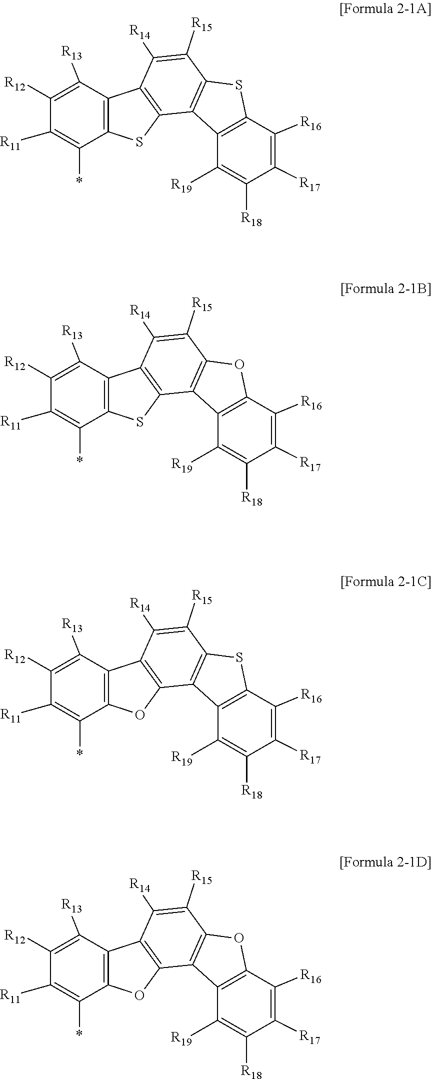
wherein in Formula 2-1A to Formula 2-1D,
R₁₁ to R₁₉ and * are the same as defined in connection with Formula 2-1.

16. The polycyclic compound of claim 13, wherein Formula 2-2 is represented by one of Formula 2-2A to Formula 2-2D: 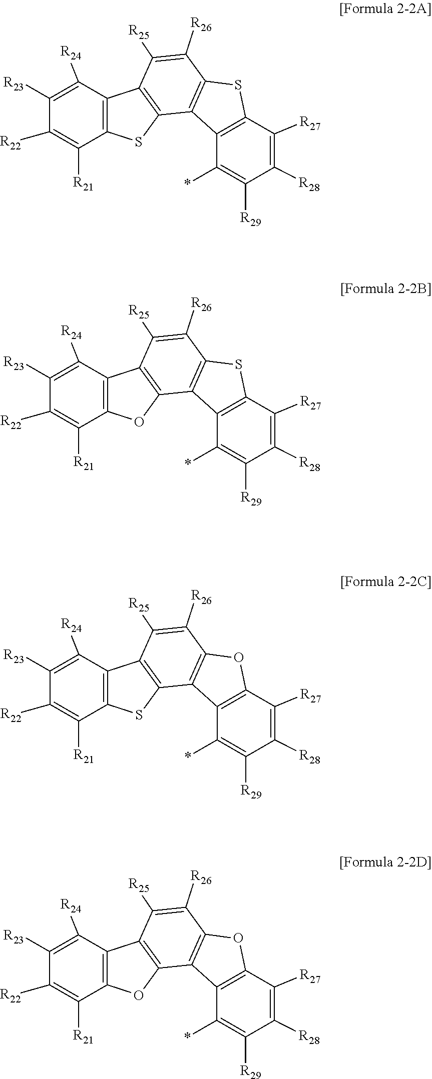
wherein in Formula 2-2A to Formula 2-2D,
R₂₁ to R₂₉ and * are the same as defined in connection with Formula 2-2.

17. The polycyclic compound of claim 13, wherein in Formula 1,
L is a direct linkage, an unsubstituted phenylene group, an unsubstituted divalent biphenyl group, an unsubstituted naphthalene group, an unsubstituted phenanthrene group, an unsubstituted dibenzofuranylene group, or an unsubstituted dibenzothiophenylene group.

18. The polycyclic compound of claim 13, wherein in Formula 2-1,
two selected from among R₁₁ to R₁₃, R₁₄ and R₁₅, or two selected from among R₁₆ to R₁₉ are bonded to each other to form a ring which is condensed with an adjacent benzene ring.

19. The polycyclic compound of claim 13, wherein in Formula 2-2,
two selected from among R₂₁ to R₂₃, R₂₄ and R₂₅, or two selected from among R₂₆ to R₂₉ are bonded to each other to form a ring which is condensed with an adjacent benzene ring.

20. The polycyclic compound of claim 13, wherein the polycyclic compound is one selected from Compound Group 1A to Compound Group 1H:

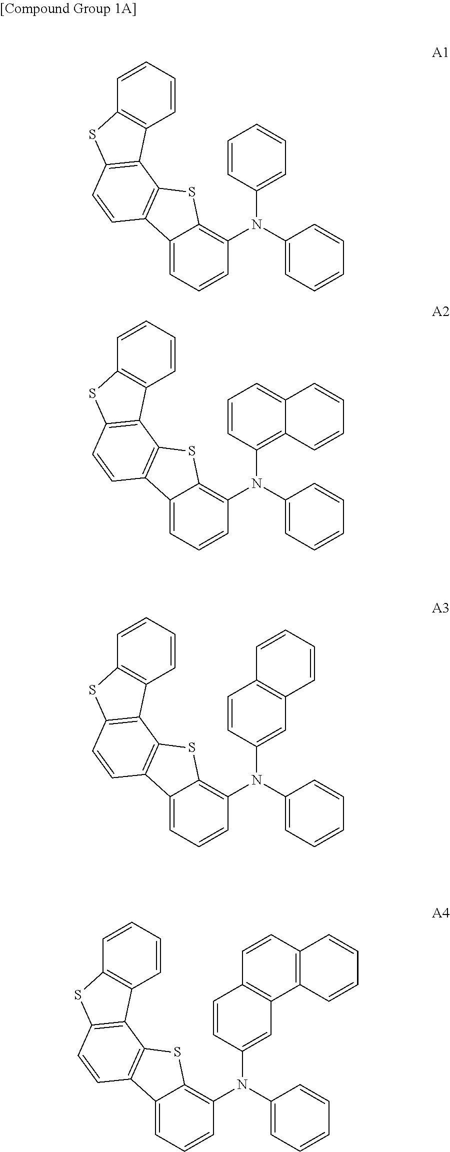 [SMILES: c1ccc(N(c2ccccc2)c2cccc3c2sc2c3ccc3sc4ccccc4c32)cc1 ; c1ccc(N(c2cccc3ccccc23)c2cccc3c2sc2c3ccc3sc4ccccc4c32)cc1 ; c1ccc(N(c2ccc3ccccc3c2)c2cccc3c2sc2c3ccc3sc4ccccc4c32)cc1 ; c1ccc(N(c2ccc3ccc4ccccc4c3c2)c2cccc3c2sc2c3ccc3sc4ccccc4c32)cc1]

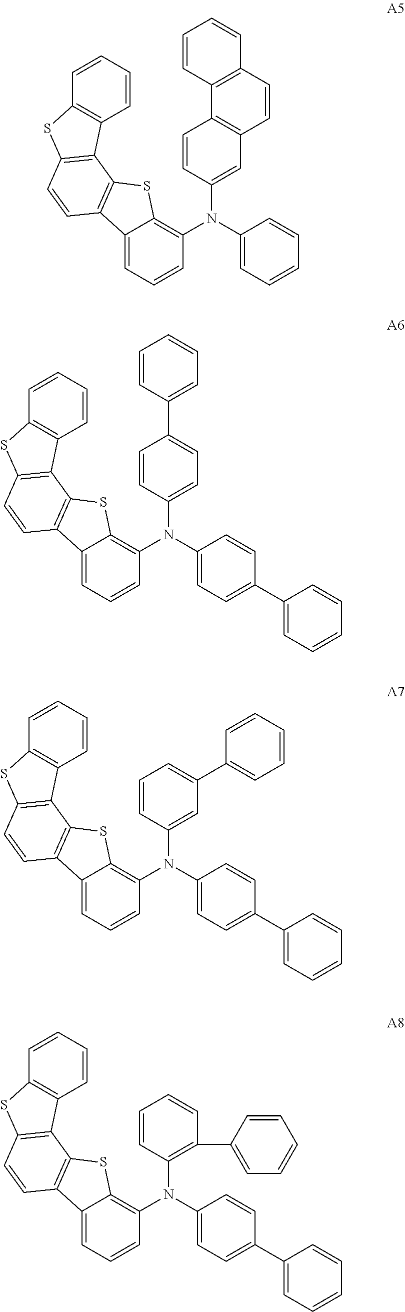 [SMILES: c1ccc(N(c2ccc3c(ccc4ccccc43)c2)c2cccc3c2sc2c3ccc3sc4ccccc4c32)cc1 ; c1ccc(-c2ccc(N(c3ccc(-c4ccccc4)cc3)c3cccc4c3sc3c4ccc4sc5ccccc5c43)cc2)cc1 ; c1ccc(-c2ccc(N(c3cccc(-c4ccccc4)c3)c3cccc4c3sc3c4ccc4sc5ccccc5c43)cc2)cc1 ; c1ccc(-c2ccc(N(c3ccccc3-c3ccccc3)c3cccc4c3sc3c4ccc4sc5ccccc5c43)cc2)cc1]

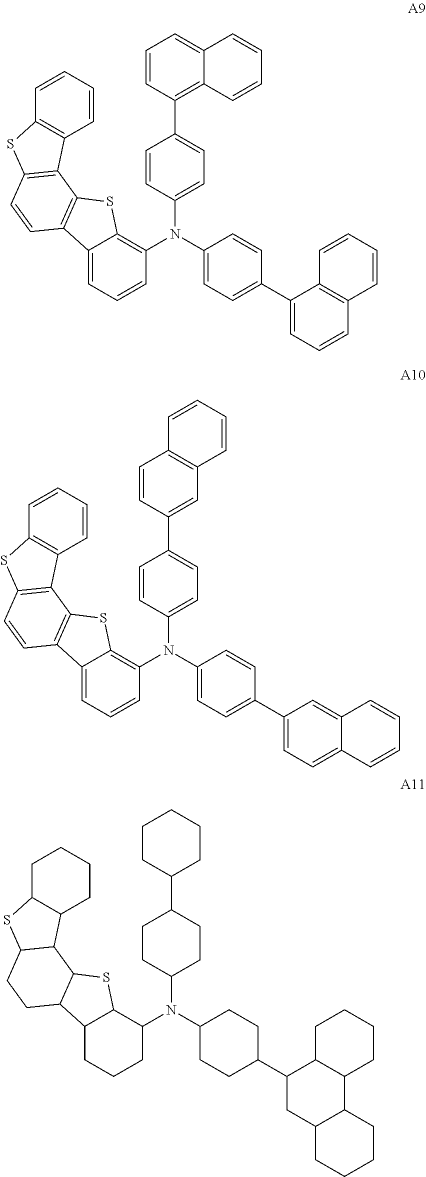 [SMILES: c1ccc2c(-c3ccc(N(c4ccc(-c5cccc6ccccc56)cc4)c4cccc5c4sc4c5ccc5sc6ccccc6c54)cc3)cccc2c1 ; c1ccc2cc(-c3ccc(N(c4ccc(-c5ccc6ccccc6c5)cc4)c4cccc5c4sc4c5ccc5sc6ccccc6c54)cc3)ccc2c1 ; C1CCC(C2CCC(N(C3CCC(C4CC5CCCCC5C5CCCCC45)CC3)C3CCCC4C5CCC6SC7CCCCC7C6C5SC43)CC2)CC1]

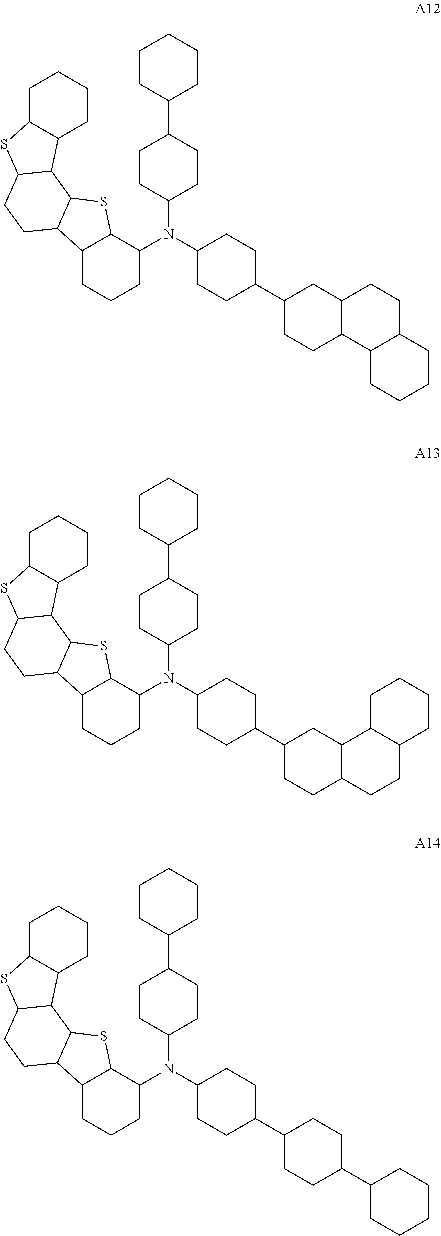 [SMILES: C1CCC(C2CCC(N(C3CCC(C4CCC5C(CCC6CCCCC65)C4)CC3)C3CCCC4C5CCC6SC7CCCCC7C6C5SC43)CC2)CC1 ; C1CCC(C2CCC(N(C3CCC(C4CCC5CCC6CCCCC6C5C4)CC3)C3CCCC4C5CCC6SC7CCCCC7C6C5SC43)CC2)CC1 ; C1CCC(C2CCC(C3CCC(N(C4CCC(C5CCCCC5)CC4)C4CCCC5C6CCC7SC8CCCCC8C7C6SC54)CC3)CC2)CC1]

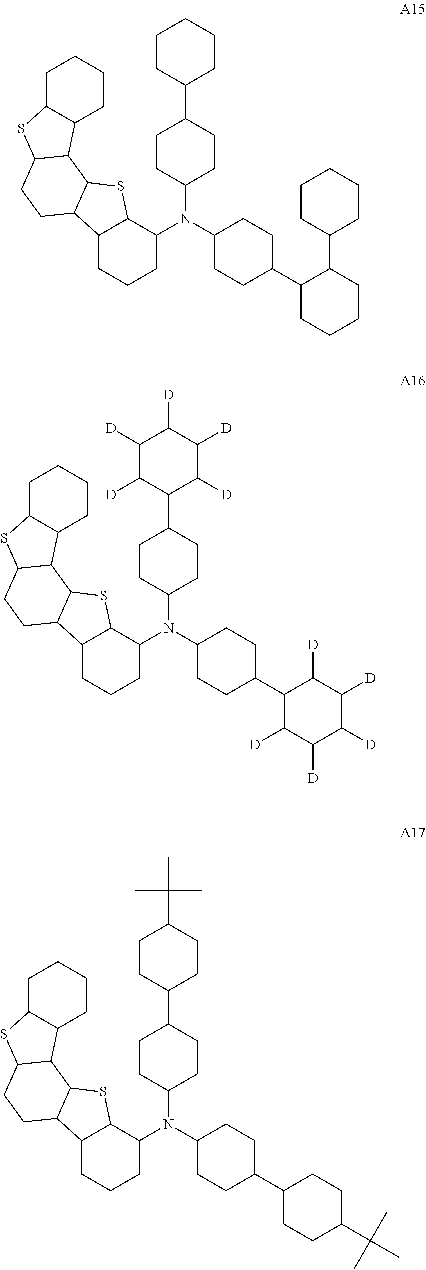 [SMILES: C1CCC(C2CCC(N(C3CCC(C4CCCCC4C4CCCCC4)CC3)C3CCCC4C5CCC6SC7CCCCC7C6C5SC43)CC2)CC1 ; [2H]C1C([2H])C([2H])C(C2CCC(N(C3CCC(C4C([2H])C([2H])C([2H])C([2H])C4[2H])CC3)C3CCCC4C5CCC6SC7CCCCC7C6C5SC43)CC2)C([2H])C1[2H] ; CC(C)(C)C1CCC(C2CCC(N(C3CCC(C4CCC(C(C)(C)C)CC4)CC3)C3CCCC4C5CCC6SC7CCCCC7C6C5SC43)CC2)CC1]

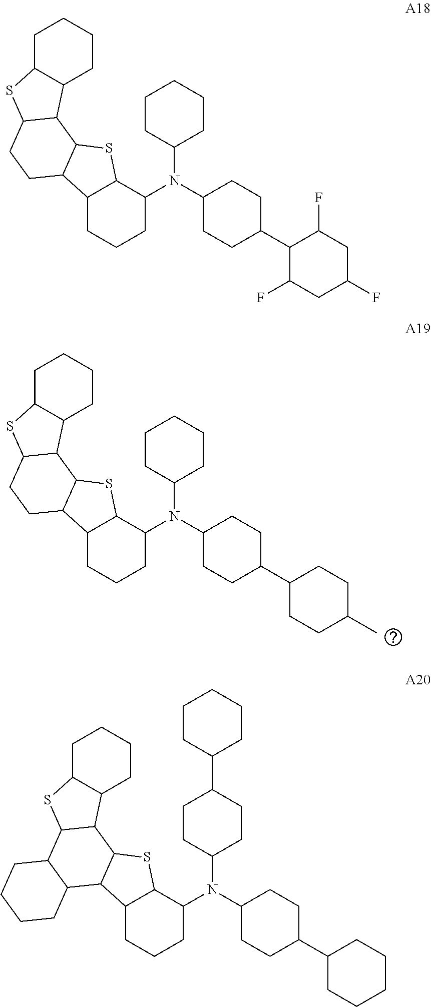 [SMILES: FC1CC(F)C(C2CCC(N(C3CCCCC3)C3CCCC4C5CCC6SC7CCCCC7C6C5SC43)CC2)C(F)C1 ; CC1CCC(C2CCC(N(C3CCCCC3)C3CCCC4C5CCC6SC7CCCCC7C6C5SC43)CC2)CC1 ; C1CCC(C2CCC(N(C3CCC(C4CCCCC4)CC3)C3CCCC4C3SC3C4C4CCCCC4C4SC5CCCCC5C43)CC2)CC1]

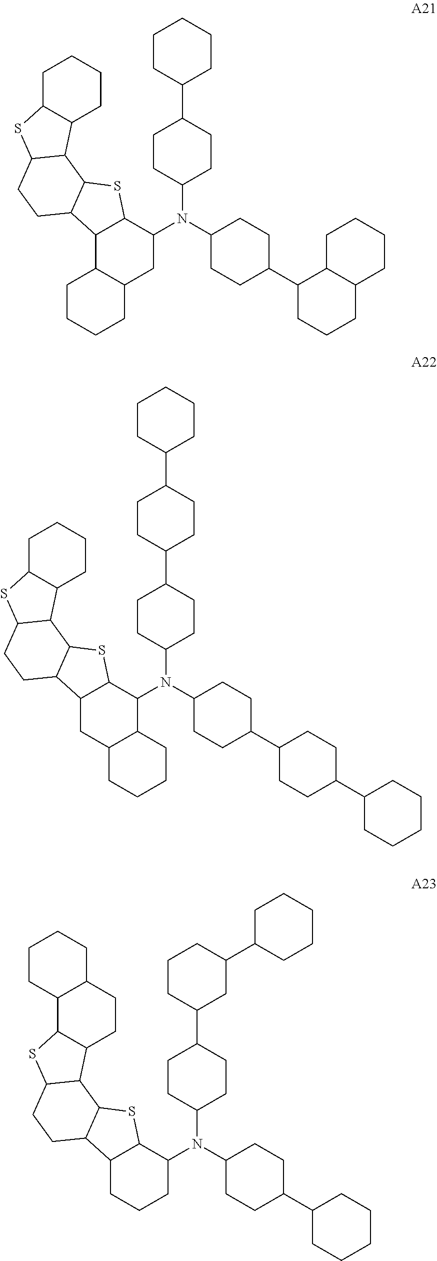 [SMILES: C1CCC(C2CCC(N(C3CCC(C4CCCC5CCCCC54)CC3)C3CC4CCCCC4C4C5CCC6SC7CCCCC7C6C5SC43)CC2)CC1 ; C1CCC(C2CCC(C3CCC(N(C4CCC(C5CCC(C6CCCCC6)CC5)CC4)C4C5CCCCC5CC5C6CCC7SC8CCCCC8C7C6SC54)CC3)CC2)CC1 ; C1CCC(C2CCC(N(C3CCC(C4CCCC(C5CCCCC5)C4)CC3)C3CCCC4C5CCC6SC7C8CCCCC8CCC7C6C5SC43)CC2)CC1]

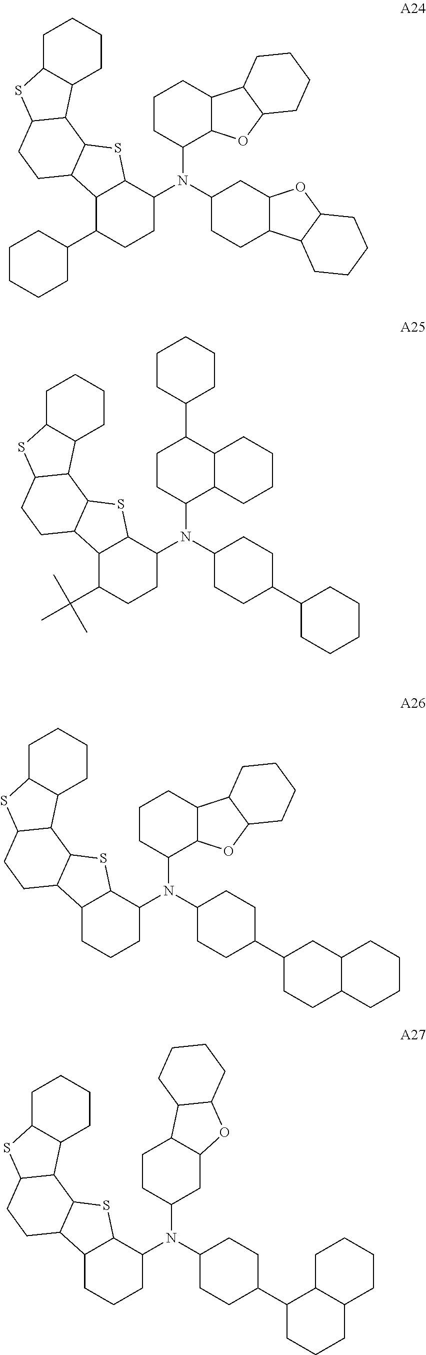 [SMILES: C1CCC(C2CCC(N(C3CCC4C(C3)OC3CCCCC34)C3CCCC4C5CCCCC5OC43)C3SC4C(CCC5SC6CCCCC6C54)C23)CC1 ; CC(C)(C)C1CCC(N(C2CCC(C3CCCCC3)CC2)C2CCC(C3CCCCC3)C3CCCCC32)C2SC3C(CCC4SC5CCCCC5C43)C21 ; C1CCC2CC(C3CCC(N(C4CCCC5C6CCCCC6OC54)C4CCCC5C6CCC7SC8CCCCC8C7C6SC54)CC3)CCC2C1 ; C1CCC2C(C1)CCCC2C1CCC(N(C2CCC3C(C2)OC2CCCCC23)C2CCCC3C4CCC5SC6CCCCC6C5C4SC32)CC1]

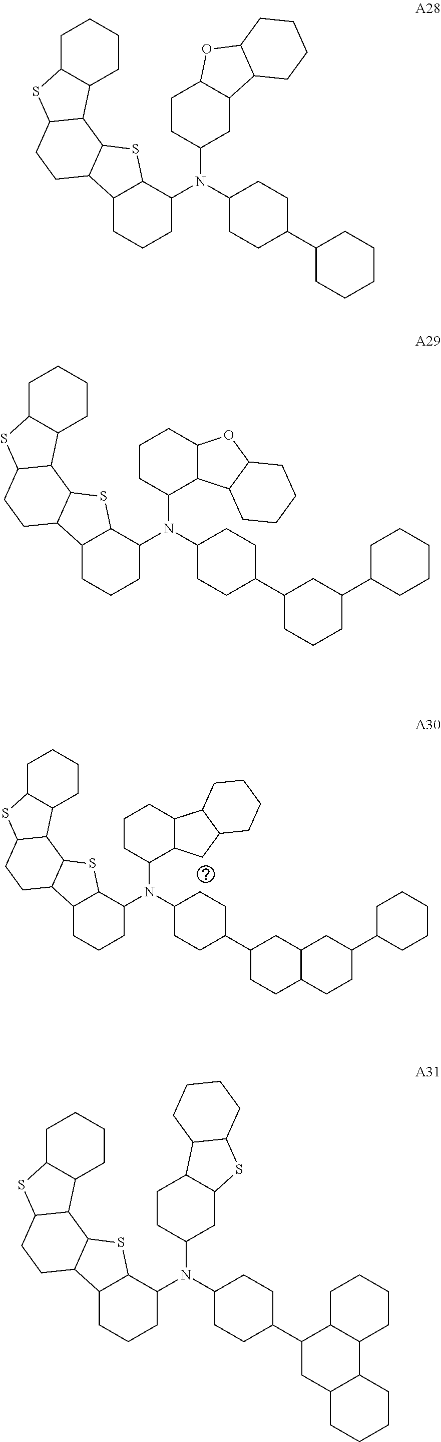 [SMILES: C1CCC(C2CCC(N(C3CCC4OC5CCCCC5C4C3)C3CCCC4C5CCC6SC7CCCCC7C6C5SC43)CC2)CC1 ; C1CCC(C2CCCC(C3CCC(N(C4CCCC5C6CCC7SC8CCCCC8C7C6SC54)C4CCCC5OC6CCCCC6C54)CC3)C2)CC1 ; C1CCC(C2CCC3CCC(C4CCC(N(C5CCCC6C7CCCCC7CC65)C5CCCC6C7CCC8SC9CCCCC9C8C7SC65)CC4)CC3C2)CC1 ; C1CCC2C(C1)CC(C1CCC(N(C3CCC4C(C3)SC3CCCCC34)C3CCCC4C5CCC6SC7CCCCC7C6C5SC43)CC1)C1CCCCC21]

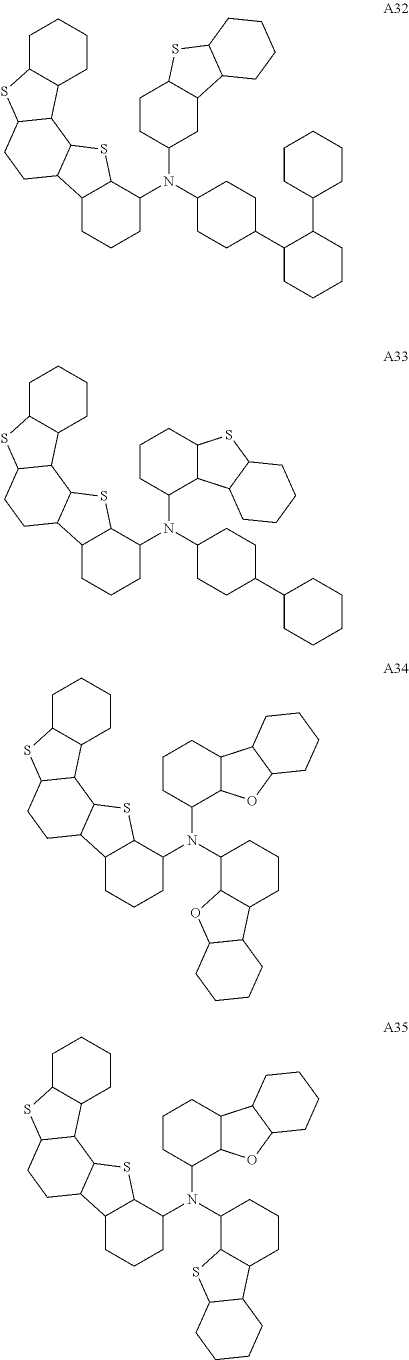 [SMILES: C1CCC(C2CCCCC2C2CCC(N(C3CCC4SC5CCCCC5C4C3)C3CCCC4C5CCC6SC7CCCCC7C6C5SC43)CC2)CC1 ; C1CCC(C2CCC(N(C3CCCC4C5CCC6SC7CCCCC7C6C5SC43)C3CCCC4SC5CCCCC5C43)CC2)CC1 ; C1CCC2C(C1)OC1C2CCCC1N(C1CCCC2C3CCCCC3OC21)C1CCCC2C3CCC4SC5CCCCC5C4C3SC21 ; C1CCC2C(C1)OC1C2CCCC1N(C1CCCC2C3CCCCC3SC21)C1CCCC2C3CCC4SC5CCCCC5C4C3SC21]

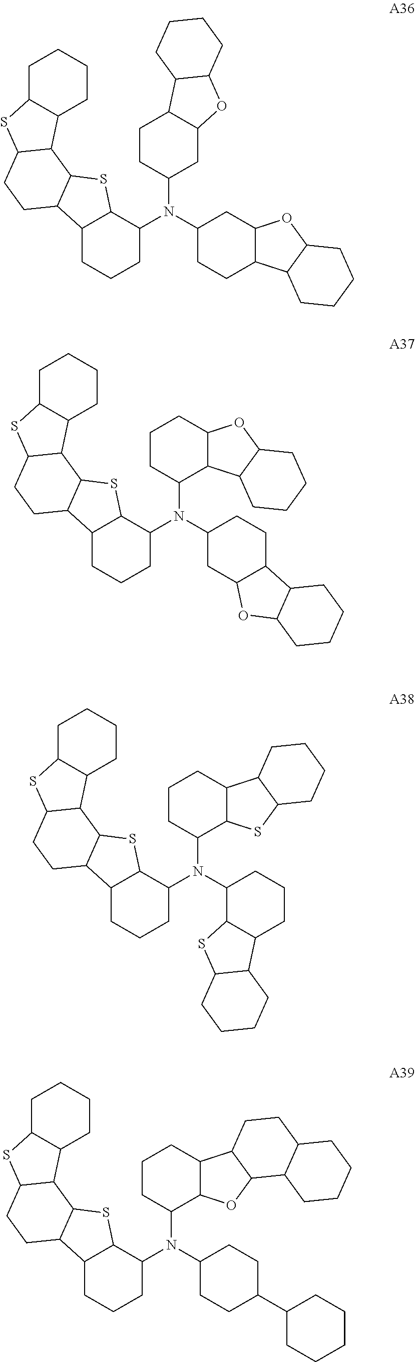 [SMILES: C1CCC2C(C1)OC1CC(N(C3CCC4C(C3)OC3CCCCC34)C3CCCC4C5CCC6SC7CCCCC7C6C5SC43)CCC12 ; C1CCC2C(C1)OC1CC(N(C3CCCC4C5CCC6SC7CCCCC7C6C5SC43)C3CCCC4OC5CCCCC5C43)CCC12 ; C1CCC2C(C1)SC1C2CCCC1N(C1CCCC2C3CCCCC3SC21)C1CCCC2C3CCC4SC5CCCCC5C4C3SC21 ; C1CCC(C2CCC(N(C3CCCC4C5CCC6CCCCC6C5OC43)C3CCCC4C5CCC6SC7CCCCC7C6C5SC43)CC2)CC1]

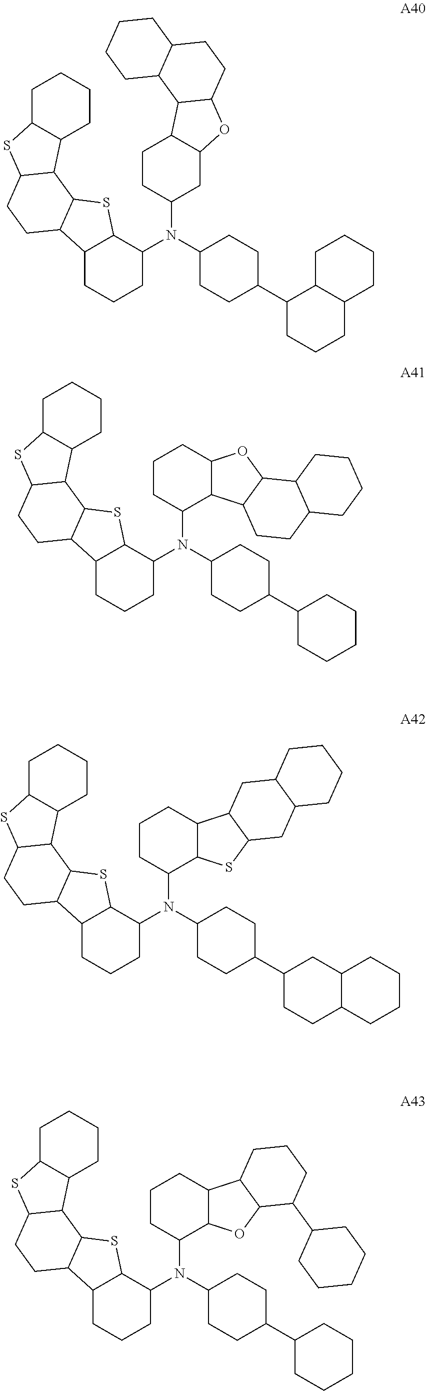 [SMILES: C1CCC2C(C1)CCCC2C1CCC(N(C2CCC3C(C2)OC2CCC4CCCCC4C23)C2CCCC3C4CCC5SC6CCCCC6C5C4SC32)CC1 ; C1CCC(C2CCC(N(C3CCCC4C5CCC6SC7CCCCC7C6C5SC43)C3CCCC4OC5C6CCCCC6CCC5C43)CC2)CC1 ; C1CCC2CC(C3CCC(N(C4CCCC5C6CC7CCCCC7CC6SC54)C4CCCC5C6CCC7SC8CCCCC8C7C6SC54)CC3)CCC2C1 ; C1CCC(C2CCC(N(C3CCCC4C5CCCC(C6CCCCC6)C5OC43)C3CCCC4C5CCC6SC7CCCCC7C6C5SC43)CC2)CC1]

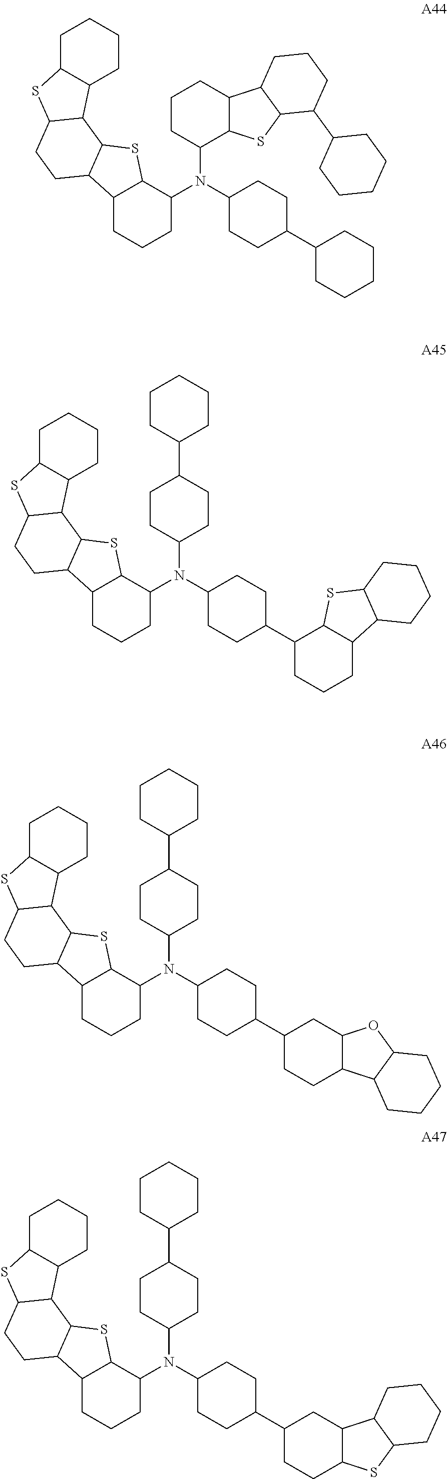 [SMILES: C1CCC(C2CCC(N(C3CCCC4C5CCCC(C6CCCCC6)C5SC43)C3CCCC4C5CCC6SC7CCCCC7C6C5SC43)CC2)CC1 ; C1CCC(C2CCC(N(C3CCC(C4CCCC5C6CCCCC6SC45)CC3)C3CCCC4C5CCC6SC7CCCCC7C6C5SC43)CC2)CC1 ; C1CCC(C2CCC(N(C3CCC(C4CCC5C(C4)OC4CCCCC45)CC3)C3CCCC4C5CCC6SC7CCCCC7C6C5SC43)CC2)CC1 ; C1CCC(C2CCC(N(C3CCC(C4CCC5SC6CCCCC6C5C4)CC3)C3CCCC4C5CCC6SC7CCCCC7C6C5SC43)CC2)CC1]

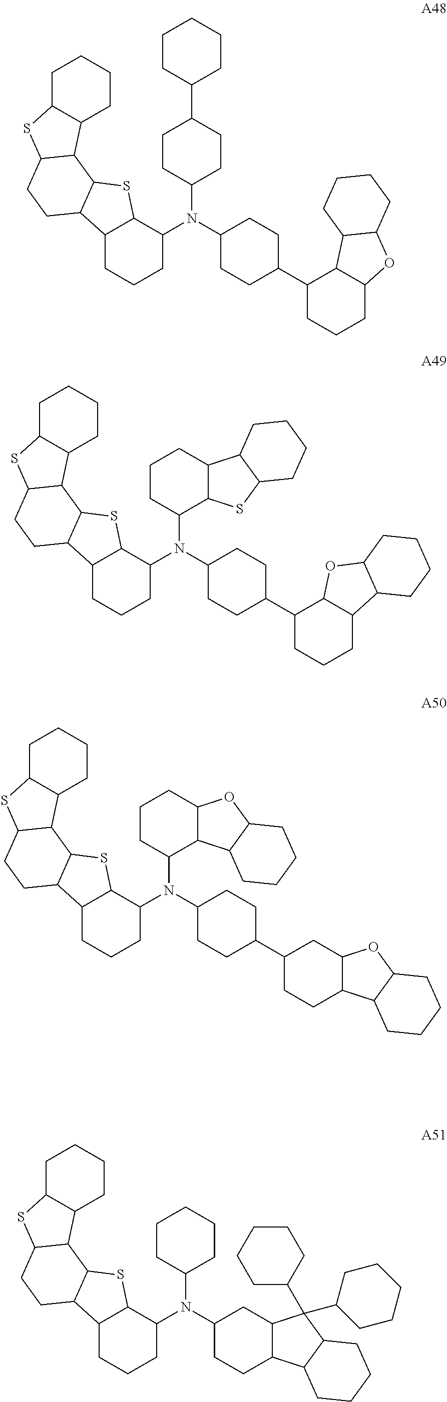 [SMILES: C1CCC(C2CCC(N(C3CCC(C4CCCC5OC6CCCCC6C54)CC3)C3CCCC4C5CCC6SC7CCCCC7C6C5SC43)CC2)CC1 ; C1CCC2C(C1)OC1C(C3CCC(N(C4CCCC5C6CCCCC6SC54)C4CCCC5C6CCC7SC8CCCCC8C7C6SC54)CC3)CCCC21 ; C1CCC2C(C1)OC1CC(C3CCC(N(C4CCCC5C6CCC7SC8CCCCC8C7C6SC54)C4CCCC5OC6CCCCC6C54)CC3)CCC12 ; C1CCC(N(C2CCC3C4CCCCC4C(C4CCCCC4)(C4CCCCC4)C3C2)C2CCCC3C4CCC5SC6CCCCC6C5C4SC32)CC1]

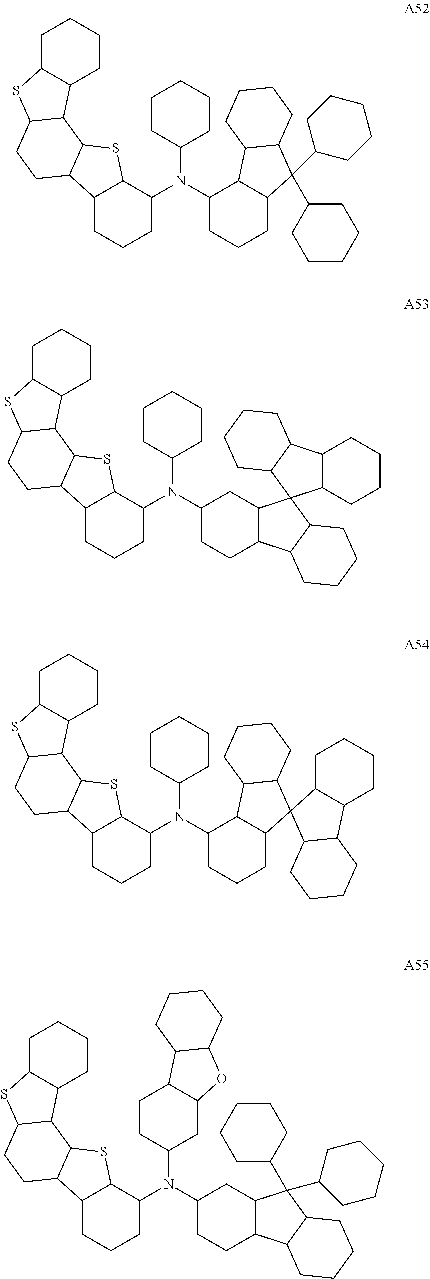 [SMILES: C1CCC(N(C2CCCC3C4CCC5SC6CCCCC6C5C4SC32)C2CCCC3C2C2CCCCC2C3(C2CCCCC2)C2CCCCC2)CC1 ; C1CCC(N(C2CCC3C4CCCCC4C4(C5CCCCC5C5CCCCC54)C3C2)C2CCCC3C4CCC5SC6CCCCC6C5C4SC32)CC1 ; C1CCC(N(C2CCCC3C4CCC5SC6CCCCC6C5C4SC32)C2CCCC3C2C2CCCCC2C32C3CCCCC3C3CCCCC32)CC1 ; C1CCC(C2(C3CCCCC3)C3CCCCC3C3CCC(N(C4CCC5C(C4)OC4CCCCC45)C4CCCC5C6CCC7SC8CCCCC8C7C6SC54)CC32)CC1]

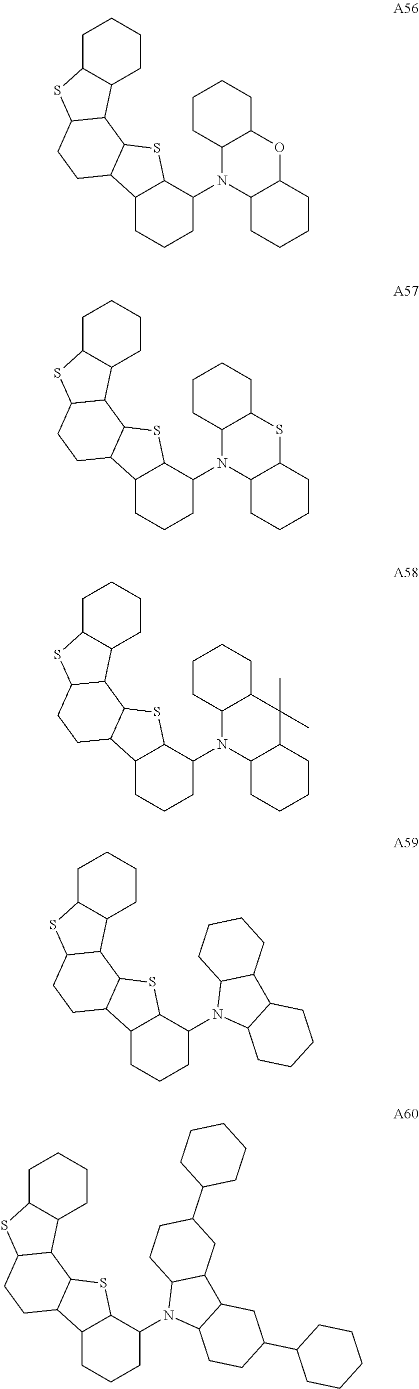 [SMILES: C1CCC2C(C1)OC1CCCCC1N2C1CCCC2C3CCC4SC5CCCCC5C4C3SC21 ; C1CCC2C(C1)SC1CCC3C4CCCC(N5C6CCCCC6SC6CCCCC65)C4SC3C12 ; CC1(C)C2CCCCC2N(C2CCCC3C4CCC5SC6CCCCC6C5C4SC32)C2CCCCC21 ; C1CCC2C(C1)SC1CCC3C4CCCC(N5C6CCCCC6C6CCCCC65)C4SC3C12 ; C1CCC(C2CCC3C(C2)C2CC(C4CCCCC4)CCC2N3C2CCCC3C4CCC5SC6CCCCC6C5C4SC32)CC1]

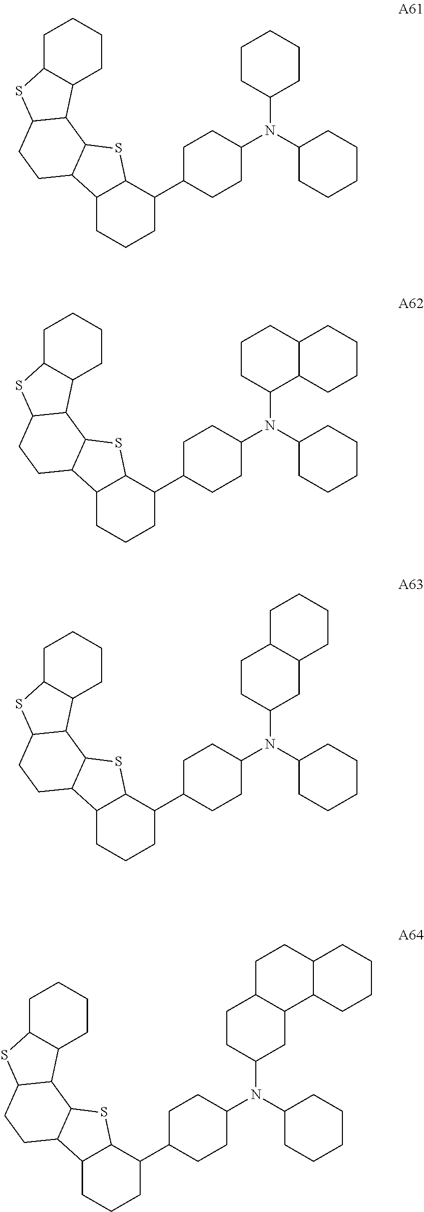 [SMILES: C1CCC(N(C2CCCCC2)C2CCC(C3CCCC4C5CCC6SC7CCCCC7C6C5SC34)CC2)CC1 ; C1CCC(N(C2CCC(C3CCCC4C5CCC6SC7CCCCC7C6C5SC34)CC2)C2CCCC3CCCCC32)CC1 ; C1CCC(N(C2CCC(C3CCCC4C5CCC6SC7CCCCC7C6C5SC34)CC2)C2CCC3CCCCC3C2)CC1 ; C1CCC(N(C2CCC(C3CCCC4C5CCC6SC7CCCCC7C6C5SC34)CC2)C2CCC3CCC4CCCCC4C3C2)CC1]

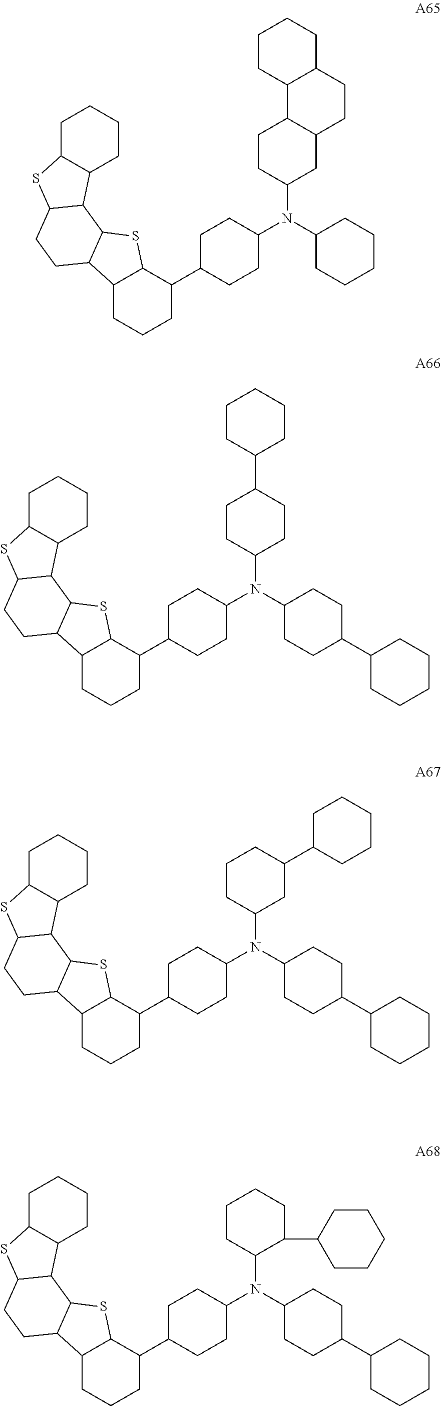 [SMILES: C1CCC(N(C2CCC(C3CCCC4C5CCC6SC7CCCCC7C6C5SC34)CC2)C2CCC3C(CCC4CCCCC43)C2)CC1 ; C1CCC(C2CCC(N(C3CCC(C4CCCCC4)CC3)C3CCC(C4CCCC5C6CCC7SC8CCCCC8C7C6SC45)CC3)CC2)CC1 ; C1CCC(C2CCC(N(C3CCC(C4CCCC5C6CCC7SC8CCCCC8C7C6SC45)CC3)C3CCCC(C4CCCCC4)C3)CC2)CC1 ; C1CCC(C2CCC(N(C3CCC(C4CCCC5C6CCC7SC8CCCCC8C7C6SC45)CC3)C3CCCCC3C3CCCCC3)CC2)CC1]

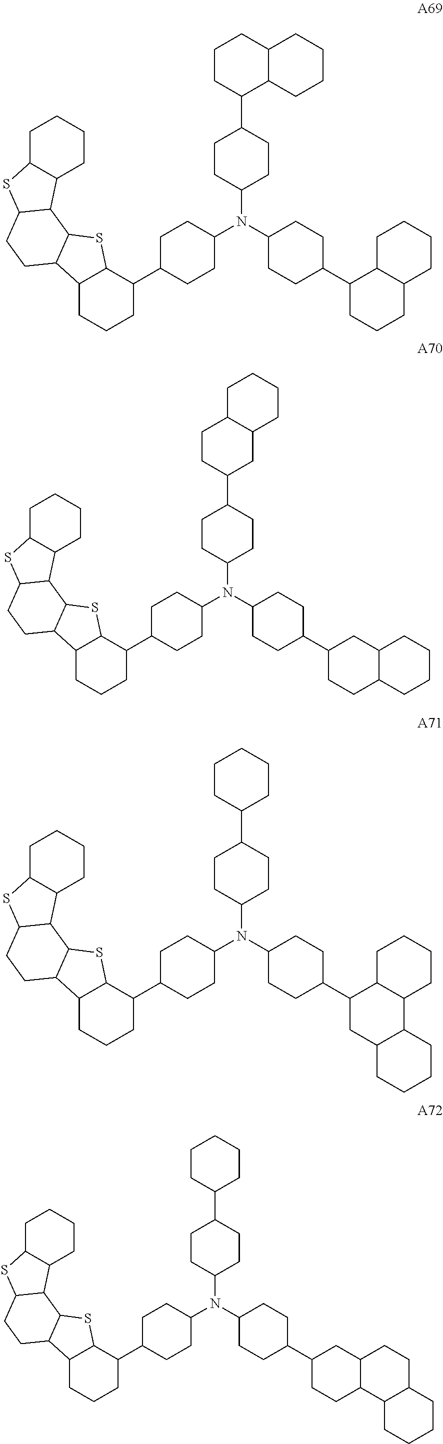 [SMILES: C1CCC2C(C1)CCCC2C1CCC(N(C2CCC(C3CCCC4CCCCC43)CC2)C2CCC(C3CCCC4C5CCC6SC7CCCCC7C6C5SC34)CC2)CC1 ; C1CCC2CC(C3CCC(N(C4CCC(C5CCC6CCCCC6C5)CC4)C4CCC(C5CCCC6C7CCC8SC9CCCCC9C8C7SC56)CC4)CC3)CCC2C1 ; C1CCC(C2CCC(N(C3CCC(C4CC5CCCCC5C5CCCCC45)CC3)C3CCC(C4CCCC5C6CCC7SC8CCCCC8C7C6SC45)CC3)CC2)CC1 ; C1CCC(C2CCC(N(C3CCC(C4CCC5C(CCC6CCCCC65)C4)CC3)C3CCC(C4CCCC5C6CCC7SC8CCCCC8C7C6SC45)CC3)CC2)CC1]

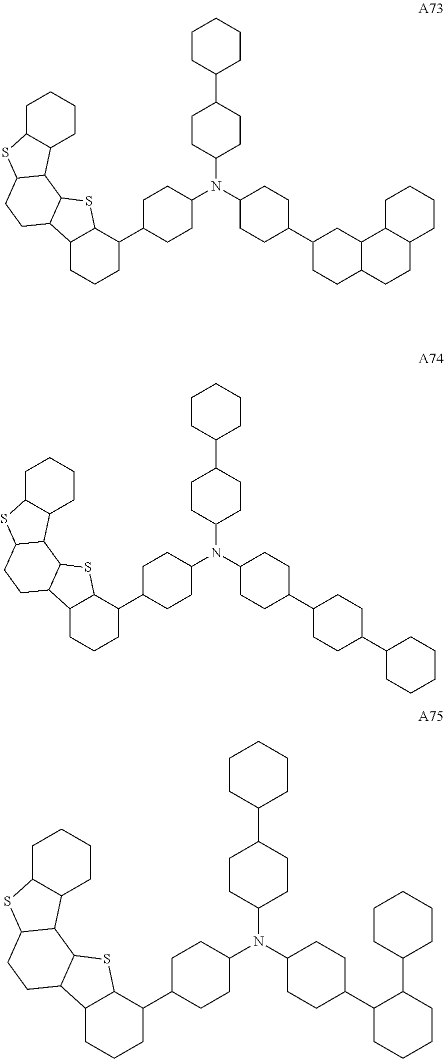 [SMILES: C1CCC(C2CCC(N(C3CCC(C4CCC5CCC6CCCCC6C5C4)CC3)C3CCC(C4CCCC5C6CCC7SC8CCCCC8C7C6SC45)CC3)CC2)CC1 ; C1CCC(C2CCC(C3CCC(N(C4CCC(C5CCCCC5)CC4)C4CCC(C5CCCC6C7CCC8SC9CCCCC9C8C7SC56)CC4)CC3)CC2)CC1 ; C1CCC(C2CCC(N(C3CCC(C4CCCCC4C4CCCCC4)CC3)C3CCC(C4CCCC5C6CCC7SC8CCCCC8C7C6SC45)CC3)CC2)CC1]

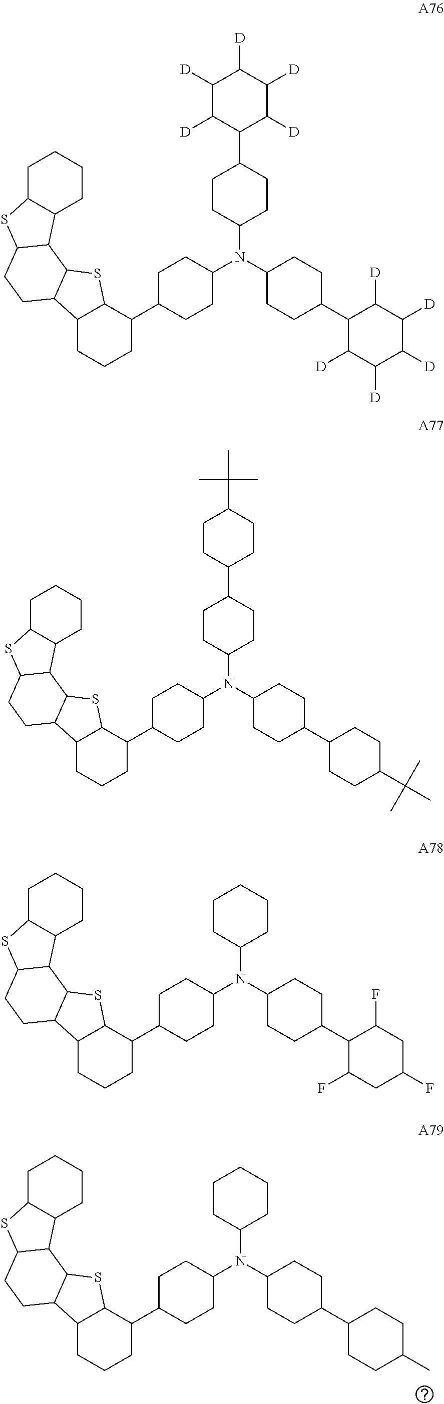 [SMILES: [2H]C1C([2H])C([2H])C(C2CCC(N(C3CCC(C4CCCC5C6CCC7SC8CCCCC8C7C6SC45)CC3)C3CCC(C4C([2H])C([2H])C([2H])C([2H])C4[2H])CC3)CC2)C([2H])C1[2H] ; CC(C)(C)C1CCC(C2CCC(N(C3CCC(C4CCC(C(C)(C)C)CC4)CC3)C3CCC(C4CCCC5C6CCC7SC8CCCCC8C7C6SC45)CC3)CC2)CC1 ; FC1CC(F)C(C2CCC(N(C3CCCCC3)C3CCC(C4CCCC5C6CCC7SC8CCCCC8C7C6SC45)CC3)CC2)C(F)C1 ; CC1CCC(C2CCC(N(C3CCCCC3)C3CCC(C4CCCC5C6CCC7SC8CCCCC8C7C6SC45)CC3)CC2)CC1]

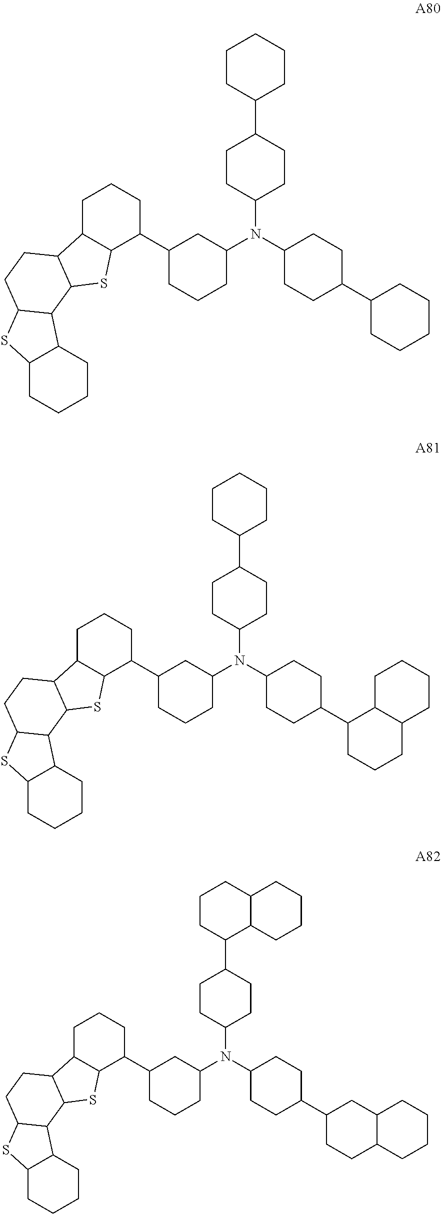 [SMILES: C1CCC(C2CCC(N(C3CCC(C4CCCCC4)CC3)C3CCCC(C4CCCC5C6CCC7SC8CCCCC8C7C6SC45)C3)CC2)CC1 ; C1CCC(C2CCC(N(C3CCC(C4CCCC5CCCCC54)CC3)C3CCCC(C4CCCC5C6CCC7SC8CCCCC8C7C6SC45)C3)CC2)CC1 ; C1CCC2CC(C3CCC(N(C4CCC(C5CCCC6CCCCC65)CC4)C4CCCC(C5CCCC6C7CCC8SC9CCCCC9C8C7SC56)C4)CC3)CCC2C1]

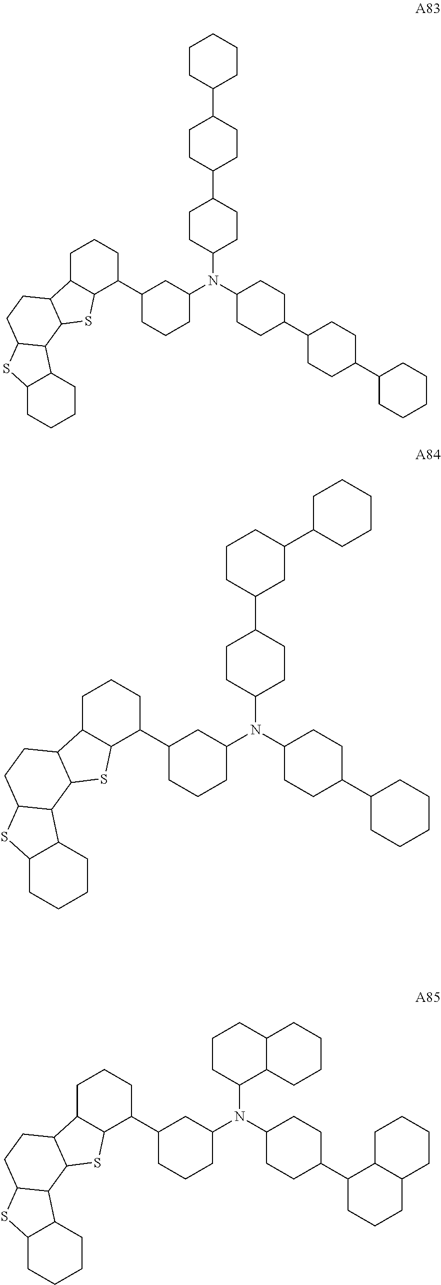 [SMILES: C1CCC(C2CCC(C3CCC(N(C4CCC(C5CCC(C6CCCCC6)CC5)CC4)C4CCCC(C5CCCC6C7CCC8SC9CCCCC9C8C7SC56)C4)CC3)CC2)CC1 ; C1CCC(C2CCC(N(C3CCC(C4CCCC(C5CCCCC5)C4)CC3)C3CCCC(C4CCCC5C6CCC7SC8CCCCC8C7C6SC45)C3)CC2)CC1 ; C1CCC2C(C1)CCCC2C1CCC(N(C2CCCC(C3CCCC4C5CCC6SC7CCCCC7C6C5SC34)C2)C2CCCC3CCCCC32)CC1]

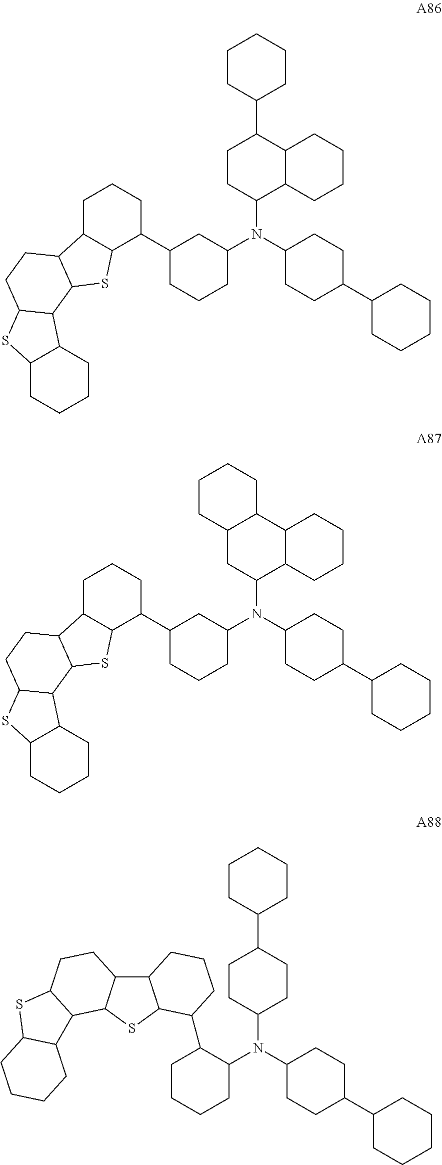 [SMILES: C1CCC(C2CCC(N(C3CCCC(C4CCCC5C6CCC7SC8CCCCC8C7C6SC45)C3)C3CCC(C4CCCCC4)C4CCCCC43)CC2)CC1 ; C1CCC(C2CCC(N(C3CCCC(C4CCCC5C6CCC7SC8CCCCC8C7C6SC45)C3)C3CC4CCCCC4C4CCCCC43)CC2)CC1 ; C1CCC(C2CCC(N(C3CCC(C4CCCCC4)CC3)C3CCCCC3C3CCCC4C5CCC6SC7CCCCC7C6C5SC43)CC2)CC1]

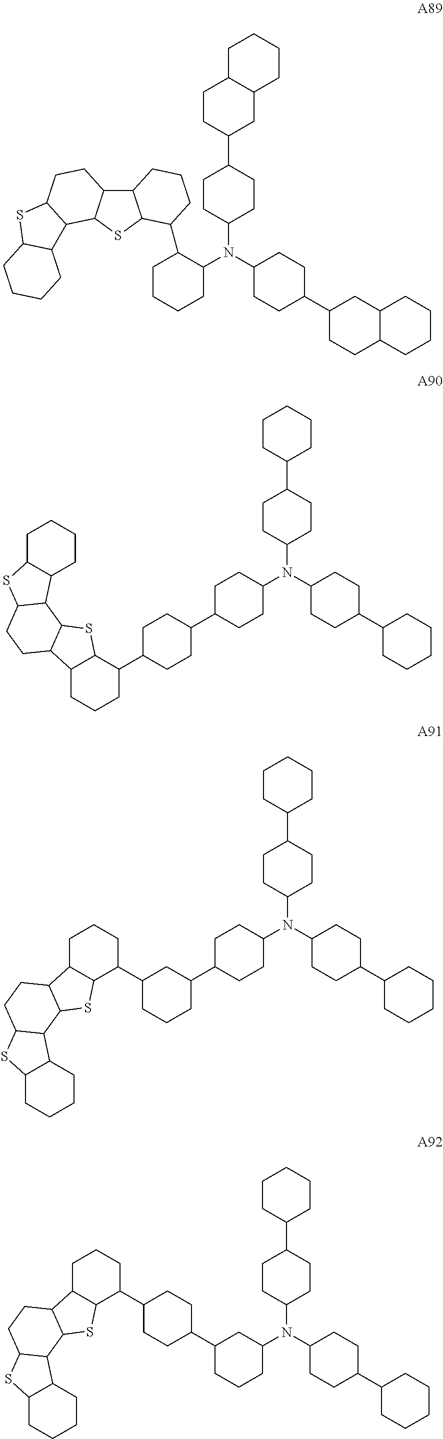 [SMILES: C1CCC2CC(C3CCC(N(C4CCC(C5CCC6CCCCC6C5)CC4)C4CCCCC4C4CCCC5C6CCC7SC8CCCCC8C7C6SC54)CC3)CCC2C1 ; C1CCC(C2CCC(N(C3CCC(C4CCCCC4)CC3)C3CCC(C4CCC(C5CCCC6C7CCC8SC9CCCCC9C8C7SC56)CC4)CC3)CC2)CC1 ; C1CCC(C2CCC(N(C3CCC(C4CCCCC4)CC3)C3CCC(C4CCCC(C5CCCC6C7CCC8SC9CCCCC9C8C7SC56)C4)CC3)CC2)CC1 ; C1CCC(C2CCC(N(C3CCC(C4CCCCC4)CC3)C3CCCC(C4CCC(C5CCCC6C7CCC8SC9CCCCC9C8C7SC56)CC4)C3)CC2)CC1]

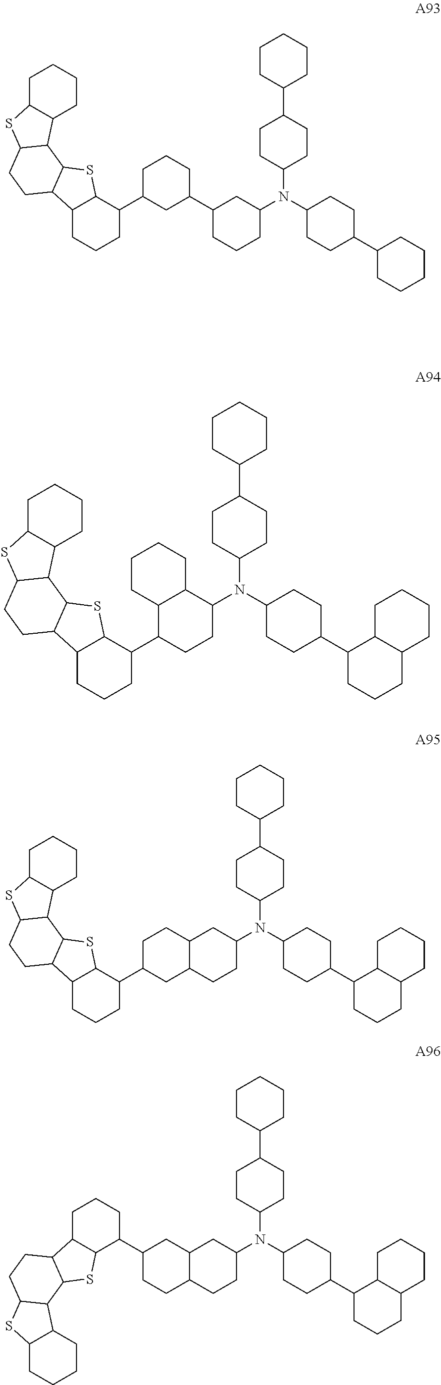 [SMILES: C1CCC(C2CCC(N(C3CCC(C4CCCCC4)CC3)C3CCCC(C4CCCC(C5CCCC6C7CCC8SC9CCCCC9C8C7SC56)C4)C3)CC2)CC1 ; C1CCC(C2CCC(N(C3CCC(C4CCCC5CCCCC54)CC3)C3CCC(C4CCCC5C6CCC7SC8CCCCC8C7C6SC45)C4CCCCC43)CC2)CC1 ; C1CCC(C2CCC(N(C3CCC(C4CCCC5CCCCC54)CC3)C3CCC4CC(C5CCCC6C7CCC8SC9CCCCC9C8C7SC56)CCC4C3)CC2)CC1 ; C1CCC(C2CCC(N(C3CCC(C4CCCC5CCCCC54)CC3)C3CCC4CCC(C5CCCC6C7CCC8SC9CCCCC9C8C7SC56)CC4C3)CC2)CC1]

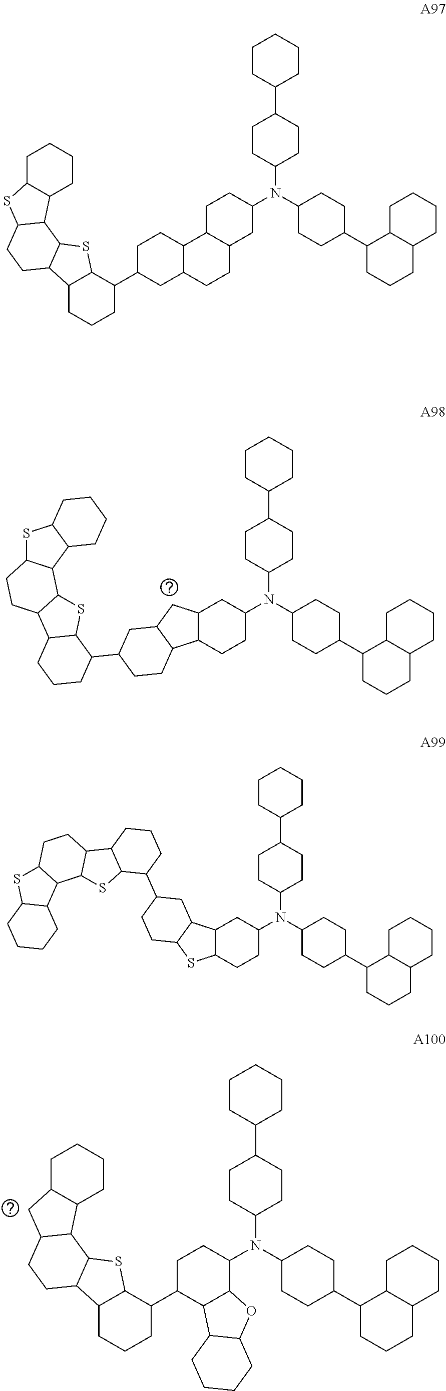 [SMILES: C1CCC(C2CCC(N(C3CCC(C4CCCC5CCCCC54)CC3)C3CCC4C(CCC5CC(C6CCCC7C8CCC9SC%10CCCCC%10C9C8SC67)CCC54)C3)CC2)CC1 ; C1CCC(C2CCC(N(C3CCC(C4CCCC5CCCCC54)CC3)C3CCC4C(CC5CC(C6CCCC7C8CCC9SC%10CCCCC%10C9C8SC67)CCC54)C3)CC2)CC1 ; C1CCC(C2CCC(N(C3CCC(C4CCCC5CCCCC54)CC3)C3CCC4SC5CCC(C6CCCC7C8CCC9SC%10CCCCC%10C9C8SC67)CC5C4C3)CC2)CC1 ; C1CCC(C2CCC(N(C3CCC(C4CCCC5CCCCC54)CC3)C3CCC(C4CCCC5C6CCC7CC8CCCCC8C7C6SC54)C4C5CCCCC5OC43)CC2)CC1]

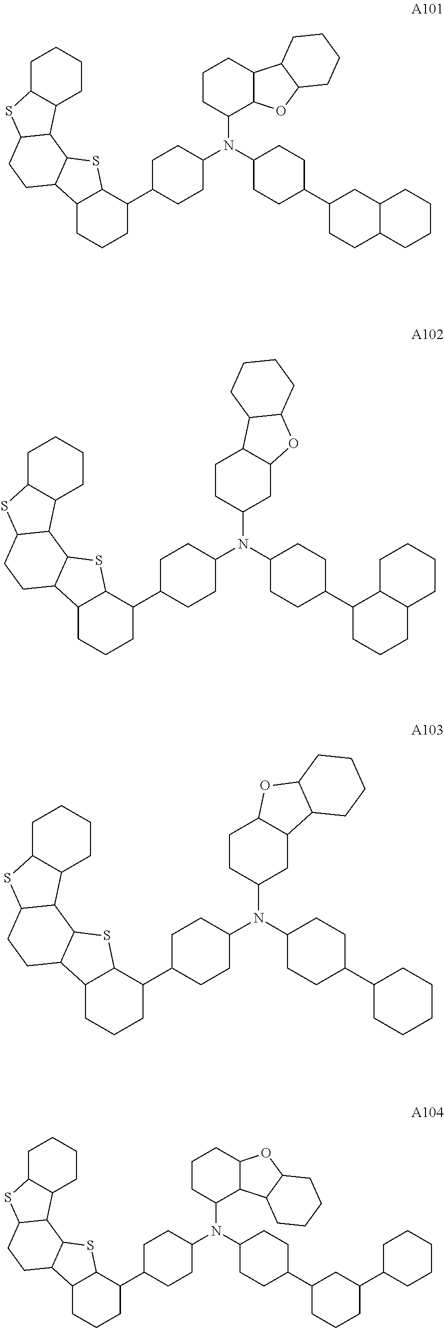 [SMILES: C1CCC2CC(C3CCC(N(C4CCC(C5CCCC6C7CCC8SC9CCCCC9C8C7SC56)CC4)C4CCCC5C6CCCCC6OC54)CC3)CCC2C1 ; C1CCC2C(C1)CCCC2C1CCC(N(C2CCC(C3CCCC4C5CCC6SC7CCCCC7C6C5SC34)CC2)C2CCC3C(C2)OC2CCCCC23)CC1 ; C1CCC(C2CCC(N(C3CCC(C4CCCC5C6CCC7SC8CCCCC8C7C6SC45)CC3)C3CCC4OC5CCCCC5C4C3)CC2)CC1 ; C1CCC(C2CCCC(C3CCC(N(C4CCC(C5CCCC6C7CCC8SC9CCCCC9C8C7SC56)CC4)C4CCCC5OC6CCCCC6C54)CC3)C2)CC1]

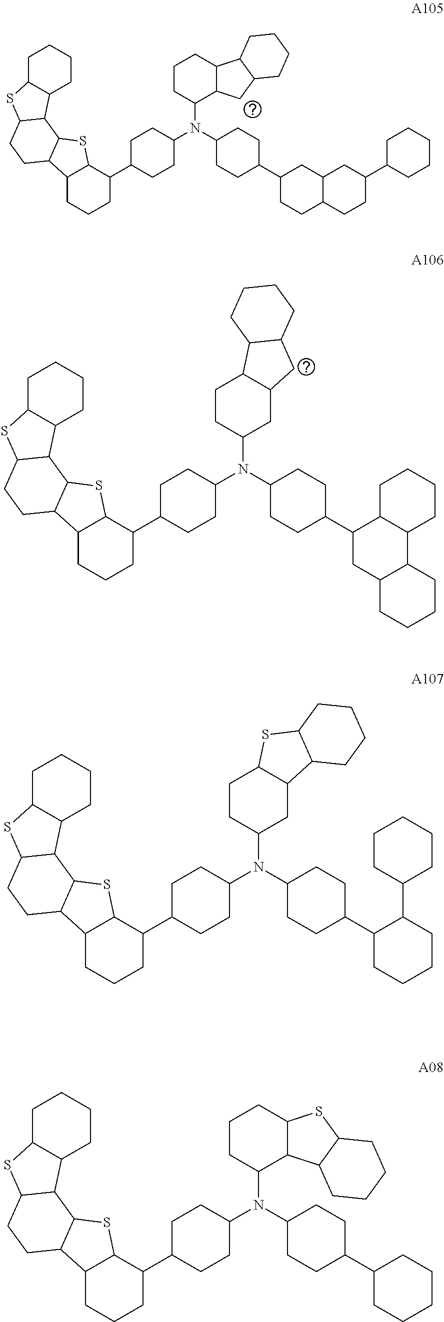 [SMILES: C1CCC(C2CCC3CCC(C4CCC(N(C5CCC(C6CCCC7C8CCC9SC%10CCCCC%10C9C8SC67)CC5)C5CCCC6C7CCCCC7CC65)CC4)CC3C2)CC1 ; C1CCC2C(C1)CC1CC(N(C3CCC(C4CC5CCCCC5C5CCCCC45)CC3)C3CCC(C4CCCC5C6CCC7SC8CCCCC8C7C6SC45)CC3)CCC12 ; C1CCC(C2CCCCC2C2CCC(N(C3CCC(C4CCCC5C6CCC7SC8CCCCC8C7C6SC45)CC3)C3CCC4SC5CCCCC5C4C3)CC2)CC1 ; C1CCC(C2CCC(N(C3CCC(C4CCCC5C6CCC7SC8CCCCC8C7C6SC45)CC3)C3CCCC4SC5CCCCC5C43)CC2)CC1]

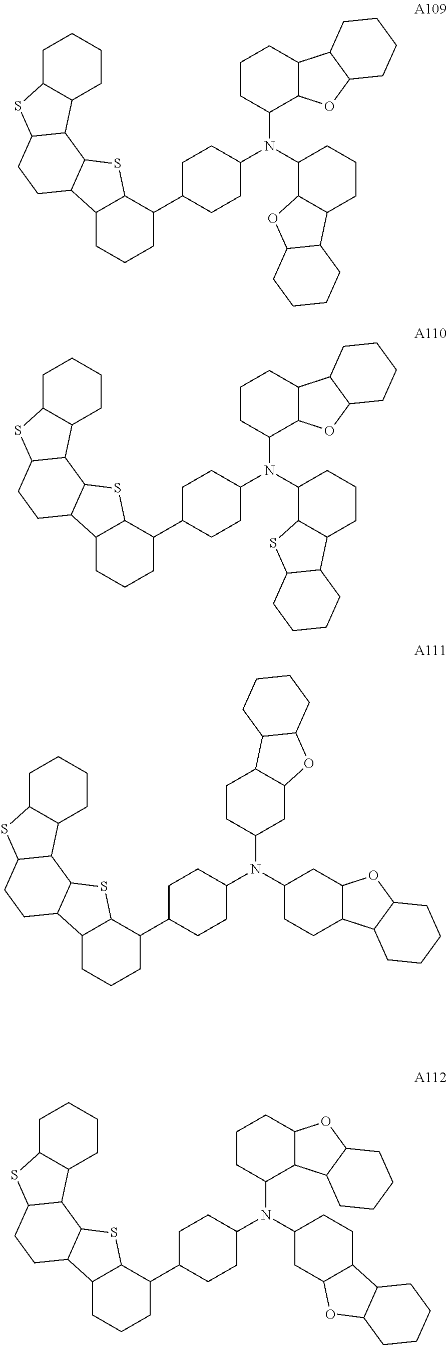

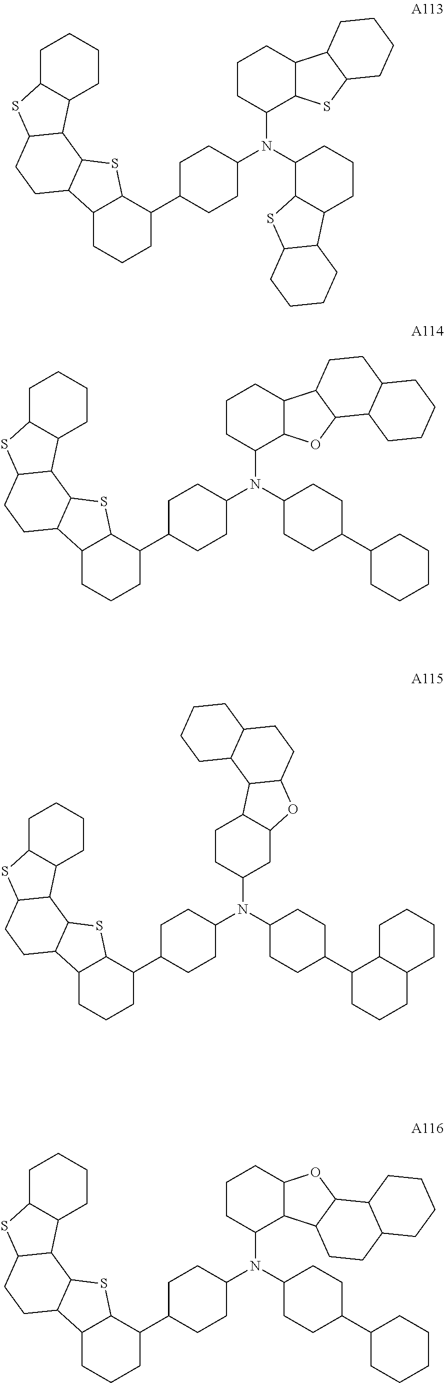 [SMILES: C1CCC2C(C1)SC1C2CCCC1N(C1CCC(C2CCCC3C4CCC5SC6CCCCC6C5C4SC23)CC1)C1CCCC2C3CCCCC3SC21 ; C1CCC(C2CCC(N(C3CCC(C4CCCC5C6CCC7SC8CCCCC8C7C6SC45)CC3)C3CCCC4C5CCC6CCCCC6C5OC43)CC2)CC1 ; C1CCC2C(C1)CCCC2C1CCC(N(C2CCC(C3CCCC4C5CCC6SC7CCCCC7C6C5SC34)CC2)C2CCC3C(C2)OC2CCC4CCCCC4C23)CC1 ; C1CCC(C2CCC(N(C3CCC(C4CCCC5C6CCC7SC8CCCCC8C7C6SC45)CC3)C3CCCC4OC5C6CCCCC6CCC5C43)CC2)CC1]

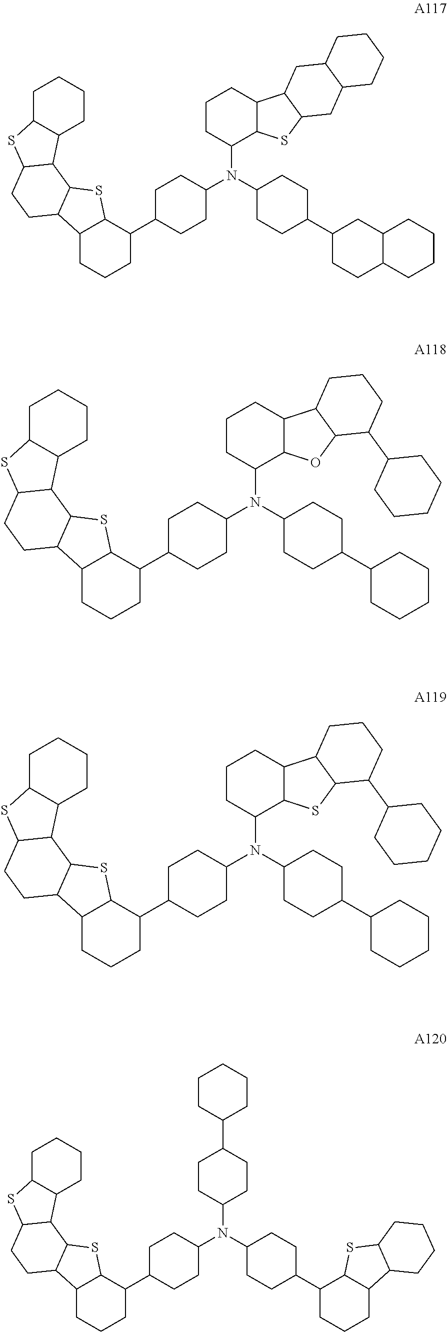 [SMILES: C1CCC2CC(C3CCC(N(C4CCC(C5CCCC6C7CCC8SC9CCCCC9C8C7SC56)CC4)C4CCCC5C6CC7CCCCC7CC6SC54)CC3)CCC2C1 ; C1CCC(C2CCC(N(C3CCC(C4CCCC5C6CCC7SC8CCCCC8C7C6SC45)CC3)C3CCCC4C5CCCC(C6CCCCC6)C5OC43)CC2)CC1 ; C1CCC(C2CCC(N(C3CCC(C4CCCC5C6CCC7SC8CCCCC8C7C6SC45)CC3)C3CCCC4C5CCCC(C6CCCCC6)C5SC43)CC2)CC1 ; C1CCC(C2CCC(N(C3CCC(C4CCCC5C6CCCCC6SC45)CC3)C3CCC(C4CCCC5C6CCC7SC8CCCCC8C7C6SC45)CC3)CC2)CC1]

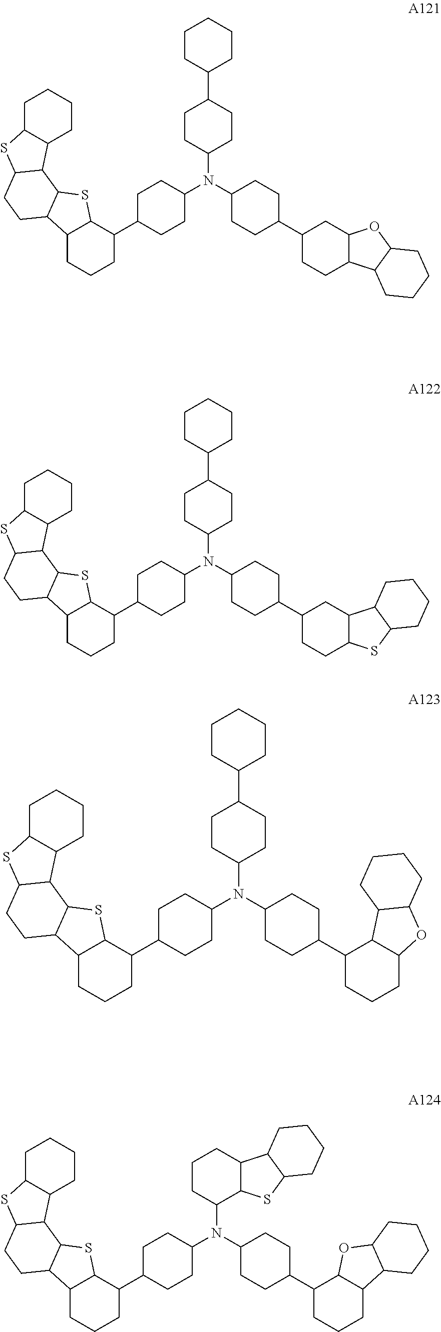 [SMILES: C1CCC(C2CCC(N(C3CCC(C4CCC5C(C4)OC4CCCCC45)CC3)C3CCC(C4CCCC5C6CCC7SC8CCCCC8C7C6SC45)CC3)CC2)CC1 ; C1CCC(C2CCC(N(C3CCC(C4CCC5SC6CCCCC6C5C4)CC3)C3CCC(C4CCCC5C6CCC7SC8CCCCC8C7C6SC45)CC3)CC2)CC1 ; C1CCC(C2CCC(N(C3CCC(C4CCCC5C6CCC7SC8CCCCC8C7C6SC45)CC3)C3CCC(C4CCCC5OC6CCCCC6C54)CC3)CC2)CC1 ; C1CCC2C(C1)OC1C(C3CCC(N(C4CCC(C5CCCC6C7CCC8SC9CCCCC9C8C7SC56)CC4)C4CCCC5C6CCCCC6SC54)CC3)CCCC21]

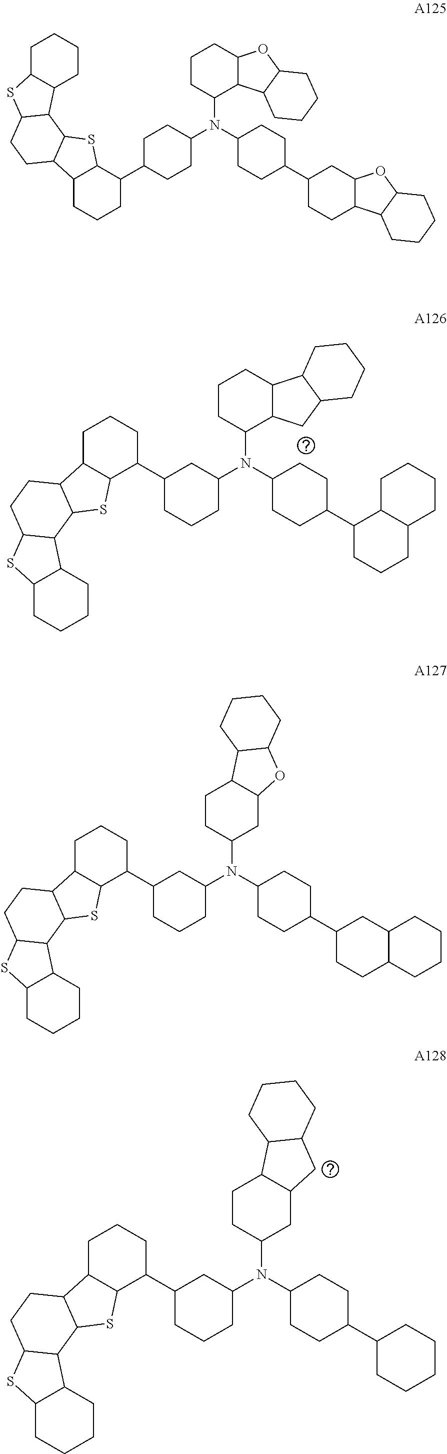 [SMILES: C1CCC2C(C1)OC1CC(C3CCC(N(C4CCC(C5CCCC6C7CCC8SC9CCCCC9C8C7SC56)CC4)C4CCCC5OC6CCCCC6C54)CC3)CCC12 ; C1CCC2C(C1)CCCC2C1CCC(N(C2CCCC(C3CCCC4C5CCC6SC7CCCCC7C6C5SC34)C2)C2CCCC3C4CCCCC4CC32)CC1 ; C1CCC2CC(C3CCC(N(C4CCCC(C5CCCC6C7CCC8SC9CCCCC9C8C7SC56)C4)C4CCC5C(C4)OC4CCCCC45)CC3)CCC2C1 ; C1CCC(C2CCC(N(C3CCC4C(CC5CCCCC54)C3)C3CCCC(C4CCCC5C6CCC7SC8CCCCC8C7C6SC45)C3)CC2)CC1]

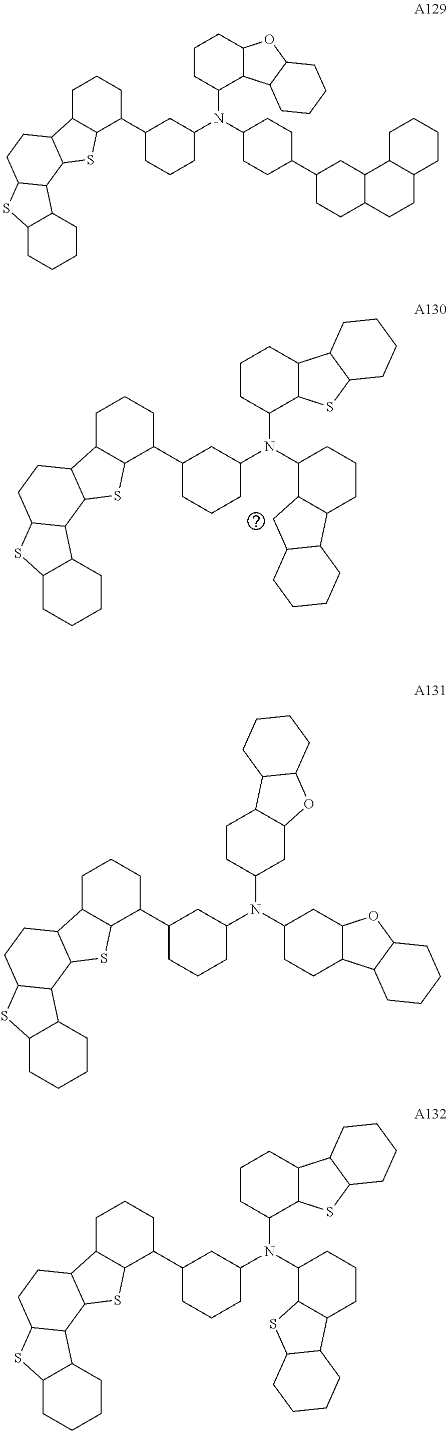 [SMILES: C1CCC2C(C1)CCC1CCC(C3CCC(N(C4CCCC(C5CCCC6C7CCC8SC9CCCCC9C8C7SC56)C4)C4CCCC5OC6CCCCC6C54)CC3)CC12 ; C1CCC2C(C1)CC1C2CCCC1N(C1CCCC(C2CCCC3C4CCC5SC6CCCCC6C5C4SC23)C1)C1CCCC2C3CCCCC3SC21 ; C1CCC2C(C1)OC1CC(N(C3CCCC(C4CCCC5C6CCC7SC8CCCCC8C7C6SC45)C3)C3CCC4C(C3)OC3CCCCC34)CCC12 ; C1CCC2C(C1)SC1C2CCCC1N(C1CCCC(C2CCCC3C4CCC5SC6CCCCC6C5C4SC23)C1)C1CCCC2C3CCCCC3SC21]

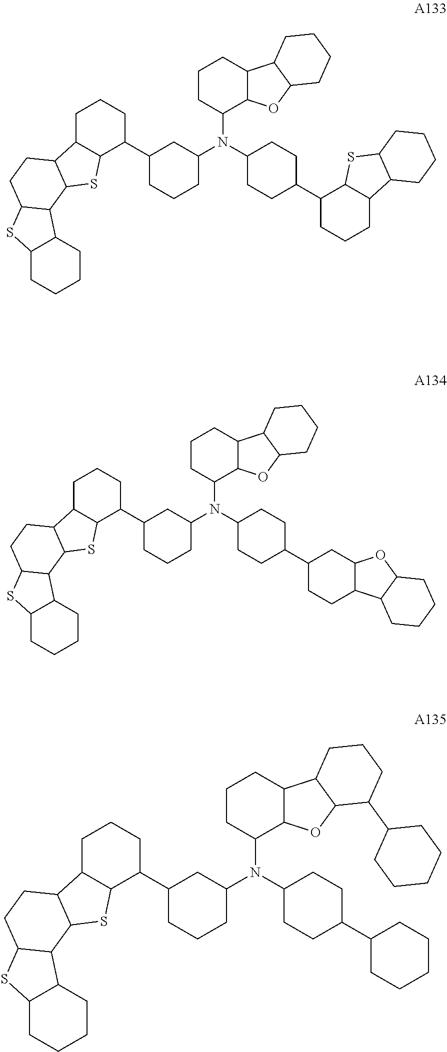 [SMILES: C1CCC2C(C1)OC1C2CCCC1N(C1CCC(C2CCCC3C4CCCCC4SC23)CC1)C1CCCC(C2CCCC3C4CCC5SC6CCCCC6C5C4SC23)C1 ; C1CCC2C(C1)OC1CC(C3CCC(N(C4CCCC(C5CCCC6C7CCC8SC9CCCCC9C8C7SC56)C4)C4CCCC5C6CCCCC6OC54)CC3)CCC12 ; C1CCC(C2CCC(N(C3CCCC(C4CCCC5C6CCC7SC8CCCCC8C7C6SC45)C3)C3CCCC4C5CCCC(C6CCCCC6)C5OC43)CC2)CC1]

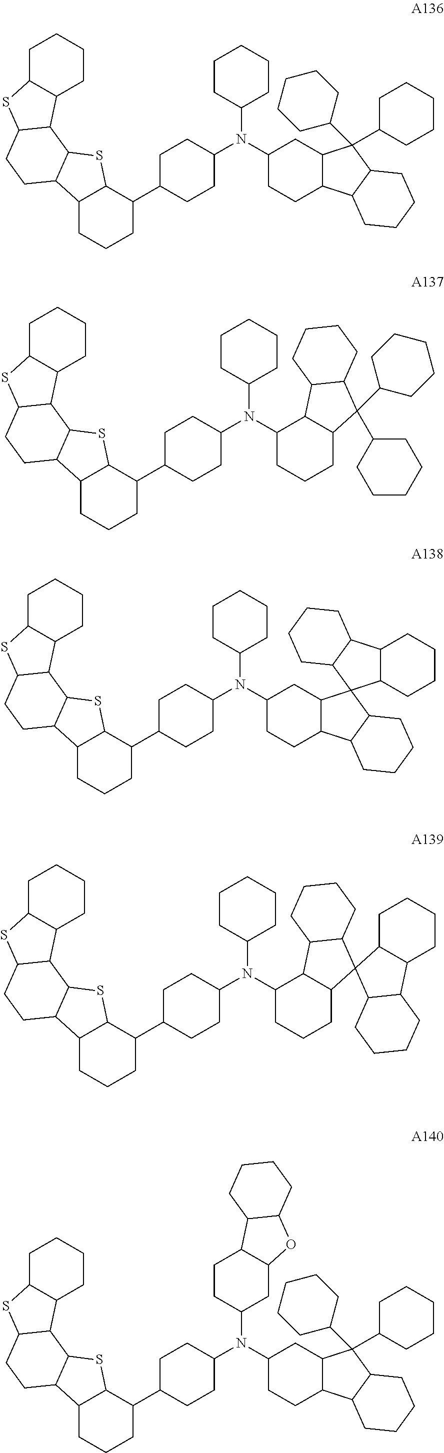 [SMILES: C1CCC(N(C2CCC(C3CCCC4C5CCC6SC7CCCCC7C6C5SC34)CC2)C2CCC3C4CCCCC4C(C4CCCCC4)(C4CCCCC4)C3C2)CC1 ; C1CCC(N(C2CCC(C3CCCC4C5CCC6SC7CCCCC7C6C5SC34)CC2)C2CCCC3C2C2CCCCC2C3(C2CCCCC2)C2CCCCC2)CC1 ; C1CCC(N(C2CCC(C3CCCC4C5CCC6SC7CCCCC7C6C5SC34)CC2)C2CCC3C4CCCCC4C4(C5CCCCC5C5CCCCC54)C3C2)CC1 ; C1CCC(N(C2CCC(C3CCCC4C5CCC6SC7CCCCC7C6C5SC34)CC2)C2CCCC3C2C2CCCCC2C32C3CCCCC3C3CCCCC32)CC1 ; C1CCC(C2(C3CCCCC3)C3CCCCC3C3CCC(N(C4CCC(C5CCCC6C7CCC8SC9CCCCC9C8C7SC56)CC4)C4CCC5C(C4)OC4CCCCC45)CC32)CC1]

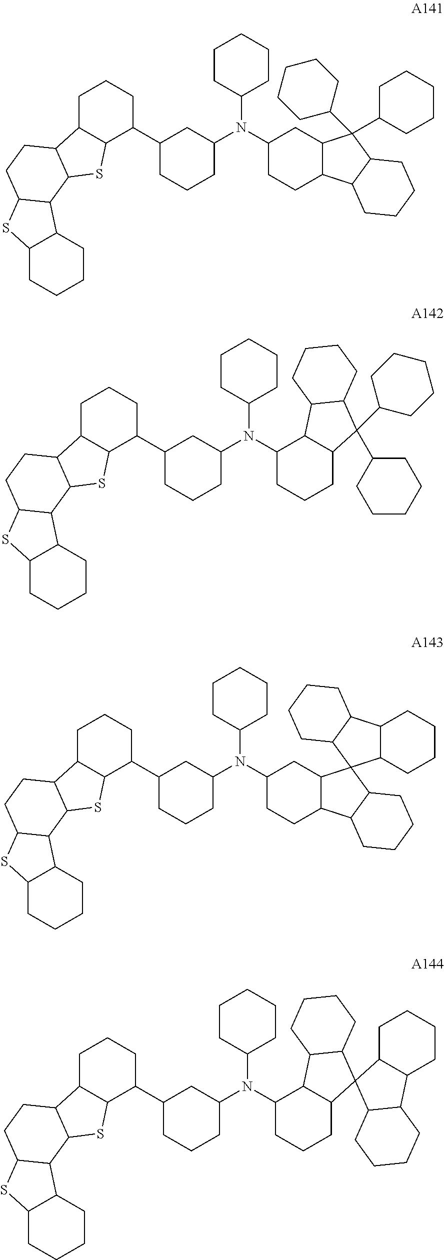 [SMILES: C1CCC(N(C2CCCC(C3CCCC4C5CCC6SC7CCCCC7C6C5SC34)C2)C2CCC3C4CCCCC4C(C4CCCCC4)(C4CCCCC4)C3C2)CC1 ; C1CCC(N(C2CCCC(C3CCCC4C5CCC6SC7CCCCC7C6C5SC34)C2)C2CCCC3C2C2CCCCC2C3(C2CCCCC2)C2CCCCC2)CC1 ; C1CCC(N(C2CCCC(C3CCCC4C5CCC6SC7CCCCC7C6C5SC34)C2)C2CCC3C4CCCCC4C4(C5CCCCC5C5CCCCC54)C3C2)CC1 ; C1CCC(N(C2CCCC(C3CCCC4C5CCC6SC7CCCCC7C6C5SC34)C2)C2CCCC3C2C2CCCCC2C32C3CCCCC3C3CCCCC32)CC1]

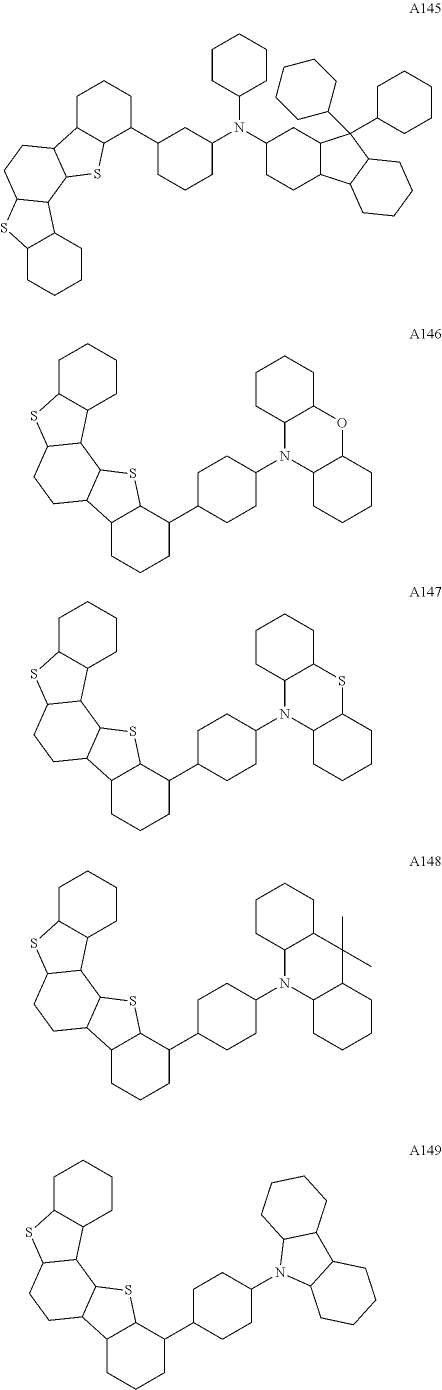 [SMILES: C1CCC(N(C2CCCC(C3CCCC4C5CCC6SC7CCCCC7C6C5SC34)C2)C2CCC3C4CCCCC4C(C4CCCCC4)(C4CCCCC4)C3C2)CC1 ; C1CCC2C(C1)OC1CCCCC1N2C1CCC(C2CCCC3C4CCC5SC6CCCCC6C5C4SC23)CC1 ; C1CCC2C(C1)SC1CCC3C4CCCC(C5CCC(N6C7CCCCC7SC7CCCCC76)CC5)C4SC3C12 ; CC1(C)C2CCCCC2N(C2CCC(C3CCCC4C5CCC6SC7CCCCC7C6C5SC34)CC2)C2CCCCC21 ; C1CCC2C(C1)SC1CCC3C4CCCC(C5CCC(N6C7CCCCC7C7CCCCC76)CC5)C4SC3C12]

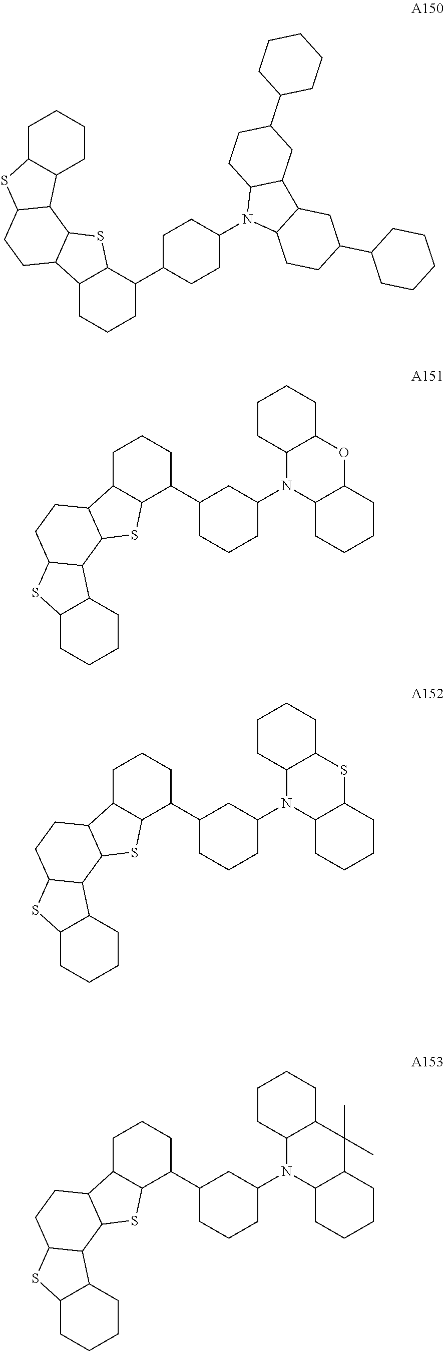 [SMILES: C1CCC(C2CCC3C(C2)C2CC(C4CCCCC4)CCC2N3C2CCC(C3CCCC4C5CCC6SC7CCCCC7C6C5SC34)CC2)CC1 ; C1CCC2C(C1)OC1CCCCC1N2C1CCCC(C2CCCC3C4CCC5SC6CCCCC6C5C4SC23)C1 ; C1CCC2C(C1)SC1CCC3C4CCCC(C5CCCC(N6C7CCCCC7SC7CCCCC76)C5)C4SC3C12 ; CC1(C)C2CCCCC2N(C2CCCC(C3CCCC4C5CCC6SC7CCCCC7C6C5SC34)C2)C2CCCCC21]

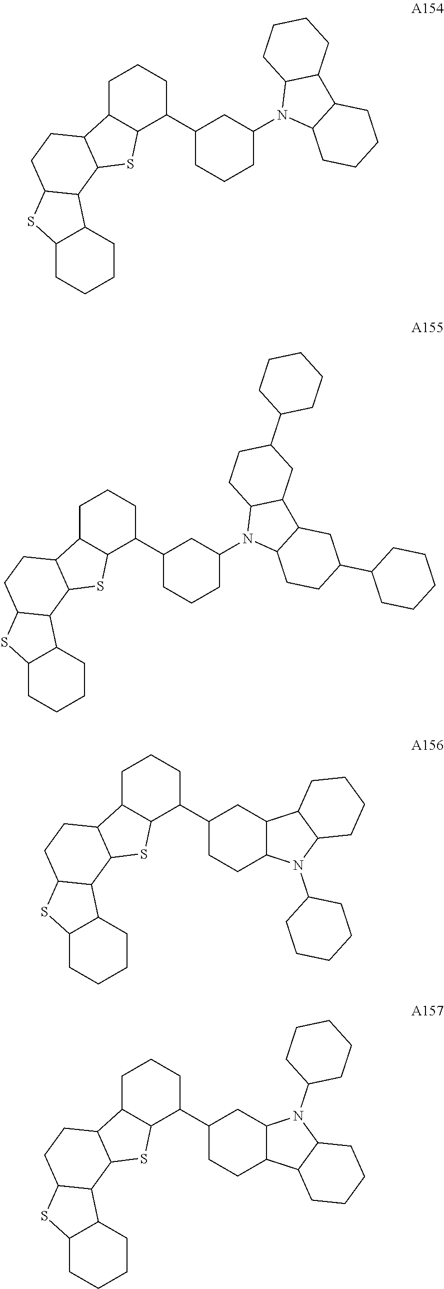 [SMILES: C1CCC2C(C1)SC1CCC3C4CCCC(C5CCCC(N6C7CCCCC7C7CCCCC76)C5)C4SC3C12 ; C1CCC(C2CCC3C(C2)C2CC(C4CCCCC4)CCC2N3C2CCCC(C3CCCC4C5CCC6SC7CCCCC7C6C5SC34)C2)CC1 ; C1CCC(N2C3CCCCC3C3CC(C4CCCC5C6CCC7SC8CCCCC8C7C6SC45)CCC32)CC1 ; C1CCC(N2C3CCCCC3C3CCC(C4CCCC5C6CCC7SC8CCCCC8C7C6SC45)CC32)CC1]

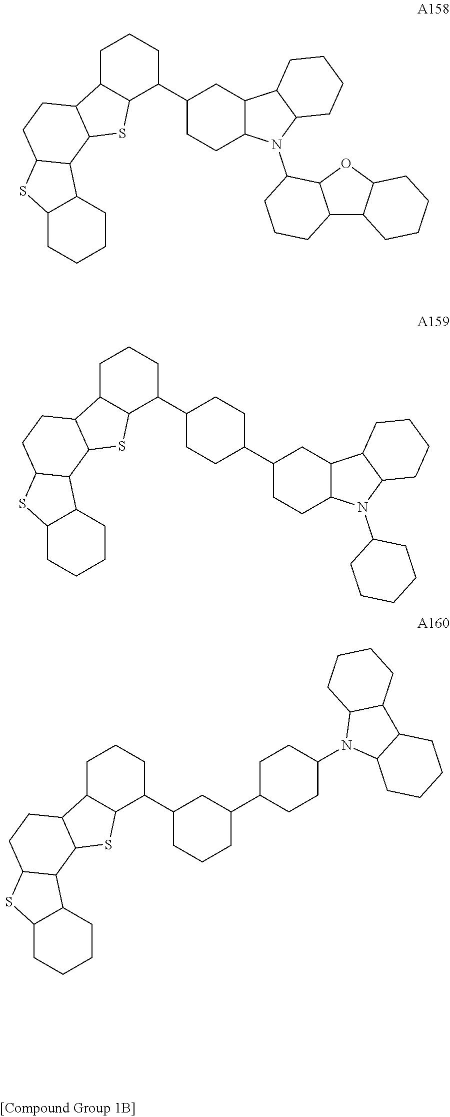 [SMILES: C1CCC2C(C1)OC1C2CCCC1N1C2CCCCC2C2CC(C3CCCC4C5CCC6SC7CCCCC7C6C5SC34)CCC21 ; C1CCC(N2C3CCCCC3C3CC(C4CCC(C5CCCC6C7CCC8SC9CCCCC9C8C7SC56)CC4)CCC32)CC1 ; C1CC(C2CCC(N3C4CCCCC4C4CCCCC43)CC2)CC(C2CCCC3C4CCC5SC6CCCCC6C5C4SC23)C1]

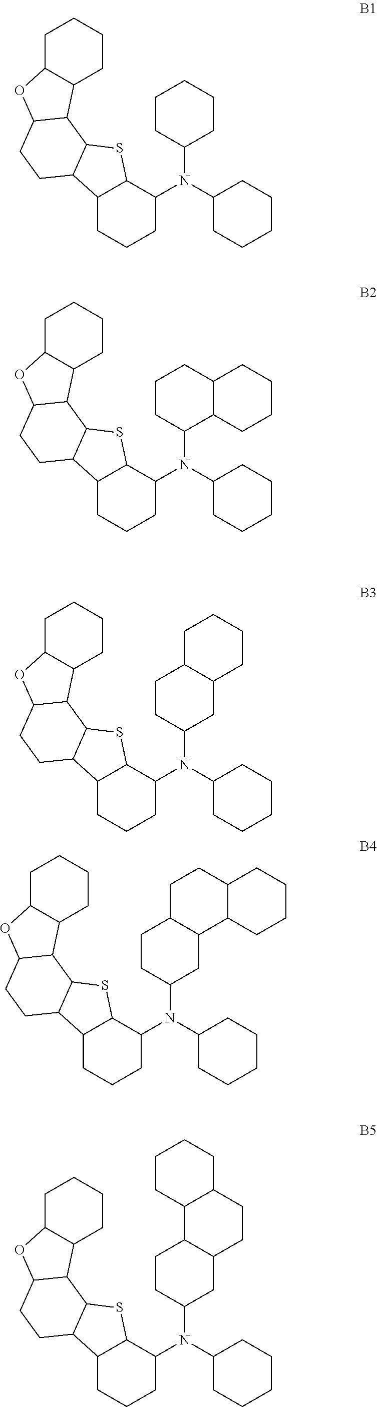

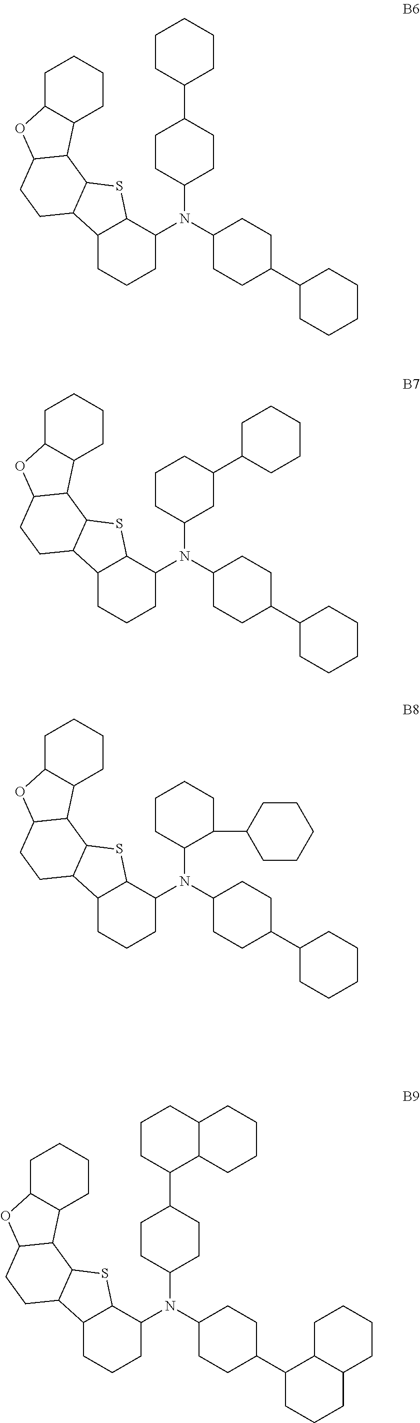 [SMILES: C1CCC(C2CCC(N(C3CCC(C4CCCCC4)CC3)C3CCCC4C5CCC6OC7CCCCC7C6C5SC43)CC2)CC1 ; C1CCC(C2CCC(N(C3CCCC(C4CCCCC4)C3)C3CCCC4C5CCC6OC7CCCCC7C6C5SC43)CC2)CC1 ; C1CCC(C2CCC(N(C3CCCCC3C3CCCCC3)C3CCCC4C5CCC6OC7CCCCC7C6C5SC43)CC2)CC1 ; C1CCC2C(C1)CCCC2C1CCC(N(C2CCC(C3CCCC4CCCCC43)CC2)C2CCCC3C4CCC5OC6CCCCC6C5C4SC32)CC1]

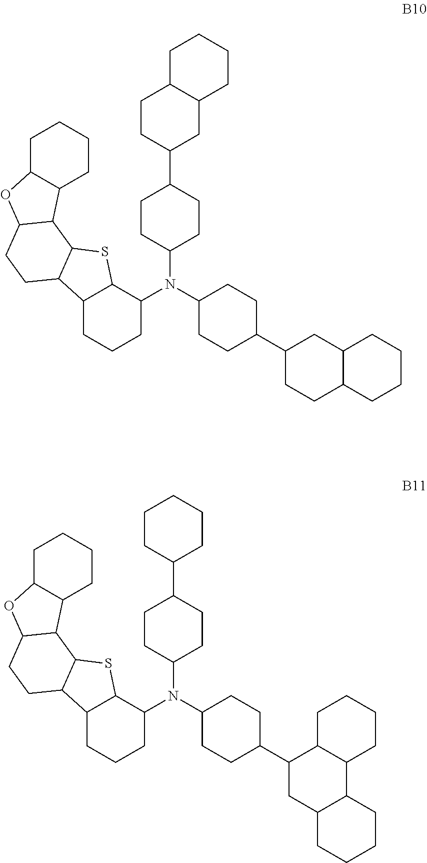 [SMILES: C1CCC2CC(C3CCC(N(C4CCC(C5CCC6CCCCC6C5)CC4)C4CCCC5C6CCC7OC8CCCCC8C7C6SC54)CC3)CCC2C1 ; C1CCC(C2CCC(N(C3CCC(C4CC5CCCCC5C5CCCCC45)CC3)C3CCCC4C5CCC6OC7CCCCC7C6C5SC43)CC2)CC1]

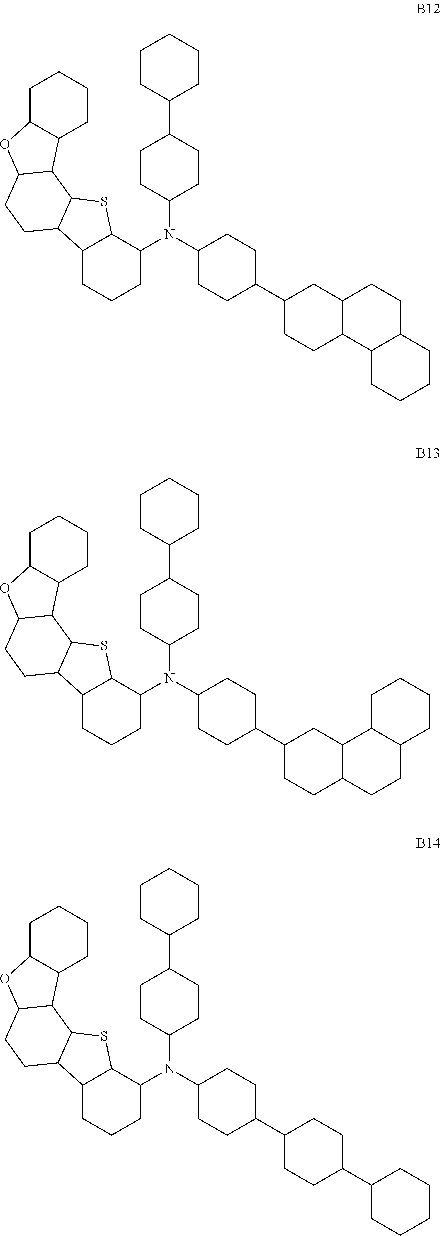 [SMILES: C1CCC(C2CCC(N(C3CCC(C4CCC5C(CCC6CCCCC65)C4)CC3)C3CCCC4C5CCC6OC7CCCCC7C6C5SC43)CC2)CC1 ; C1CCC(C2CCC(N(C3CCC(C4CCC5CCC6CCCCC6C5C4)CC3)C3CCCC4C5CCC6OC7CCCCC7C6C5SC43)CC2)CC1 ; C1CCC(C2CCC(C3CCC(N(C4CCC(C5CCCCC5)CC4)C4CCCC5C6CCC7OC8CCCCC8C7C6SC54)CC3)CC2)CC1]

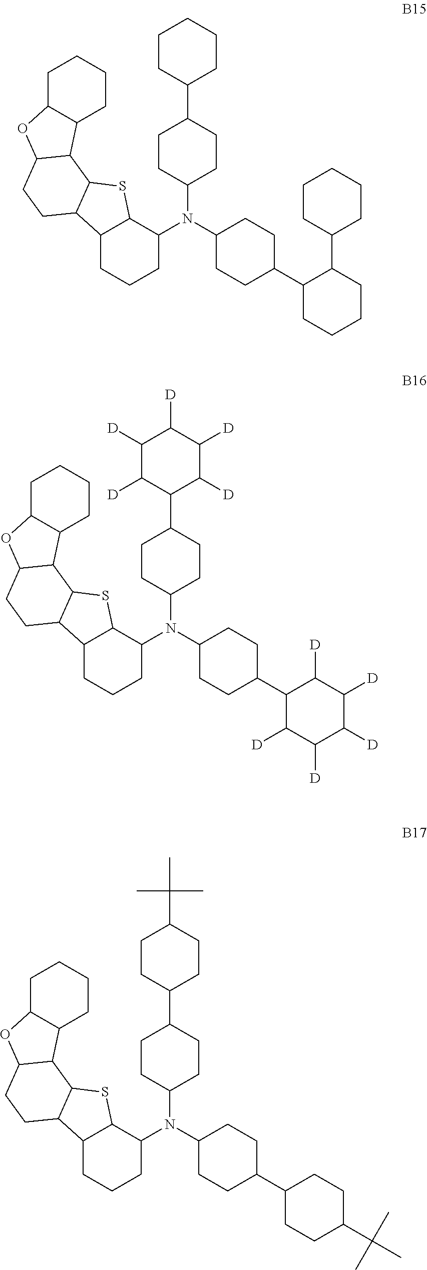 [SMILES: C1CCC(C2CCC(N(C3CCC(C4CCCCC4C4CCCCC4)CC3)C3CCCC4C5CCC6OC7CCCCC7C6C5SC43)CC2)CC1 ; [2H]C1C([2H])C([2H])C(C2CCC(N(C3CCC(C4C([2H])C([2H])C([2H])C([2H])C4[2H])CC3)C3CCCC4C5CCC6OC7CCCCC7C6C5SC43)CC2)C([2H])C1[2H] ; CC(C)(C)C1CCC(C2CCC(N(C3CCC(C4CCC(C(C)(C)C)CC4)CC3)C3CCCC4C5CCC6OC7CCCCC7C6C5SC43)CC2)CC1]

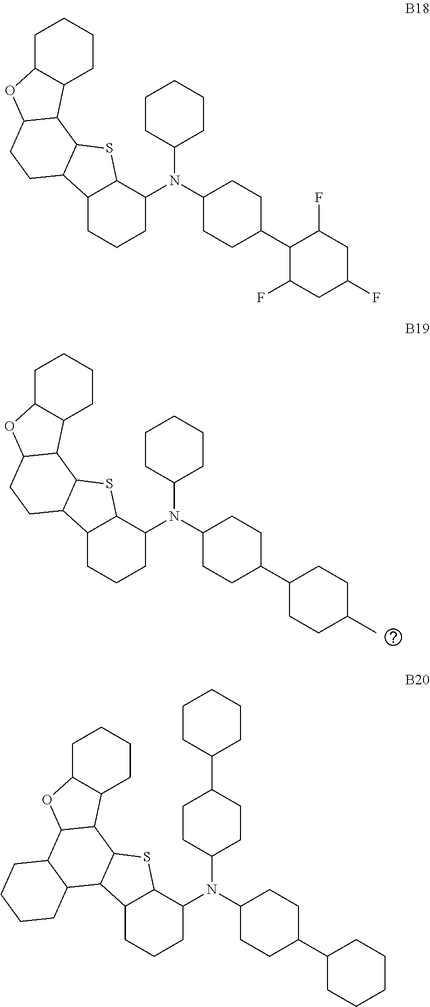 [SMILES: FC1CC(F)C(C2CCC(N(C3CCCCC3)C3CCCC4C5CCC6OC7CCCCC7C6C5SC43)CC2)C(F)C1 ; CC1CCC(C2CCC(N(C3CCCCC3)C3CCCC4C5CCC6OC7CCCCC7C6C5SC43)CC2)CC1 ; C1CCC(C2CCC(N(C3CCC(C4CCCCC4)CC3)C3CCCC4C3SC3C4C4CCCCC4C4OC5CCCCC5C43)CC2)CC1]

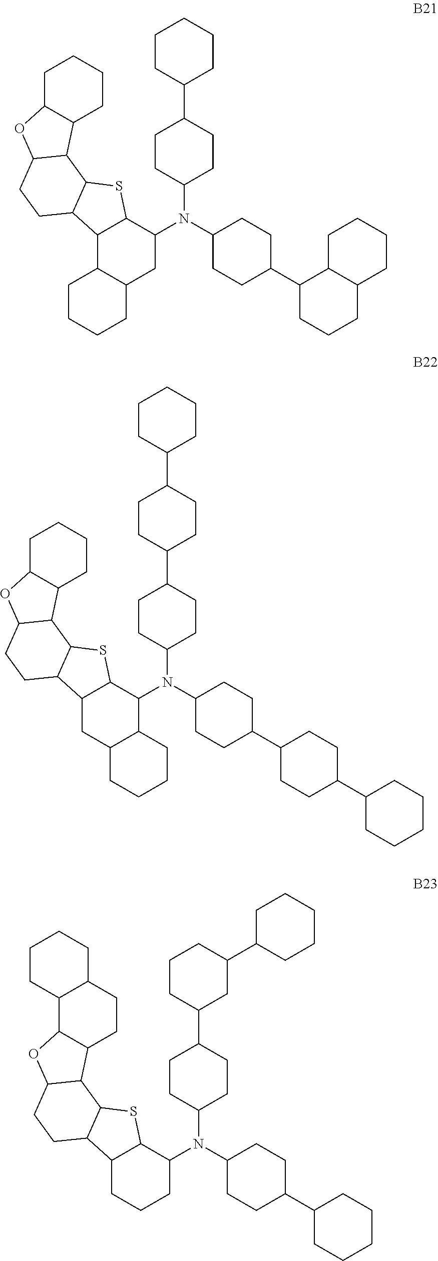 [SMILES: C1CCC(C2CCC(N(C3CCC(C4CCCC5CCCCC54)CC3)C3CC4CCCCC4C4C5CCC6OC7CCCCC7C6C5SC43)CC2)CC1 ; C1CCC(C2CCC(C3CCC(N(C4CCC(C5CCC(C6CCCCC6)CC5)CC4)C4C5CCCCC5CC5C6CCC7OC8CCCCC8C7C6SC54)CC3)CC2)CC1 ; C1CCC(C2CCC(N(C3CCC(C4CCCC(C5CCCCC5)C4)CC3)C3CCCC4C5CCC6OC7C8CCCCC8CCC7C6C5SC43)CC2)CC1]

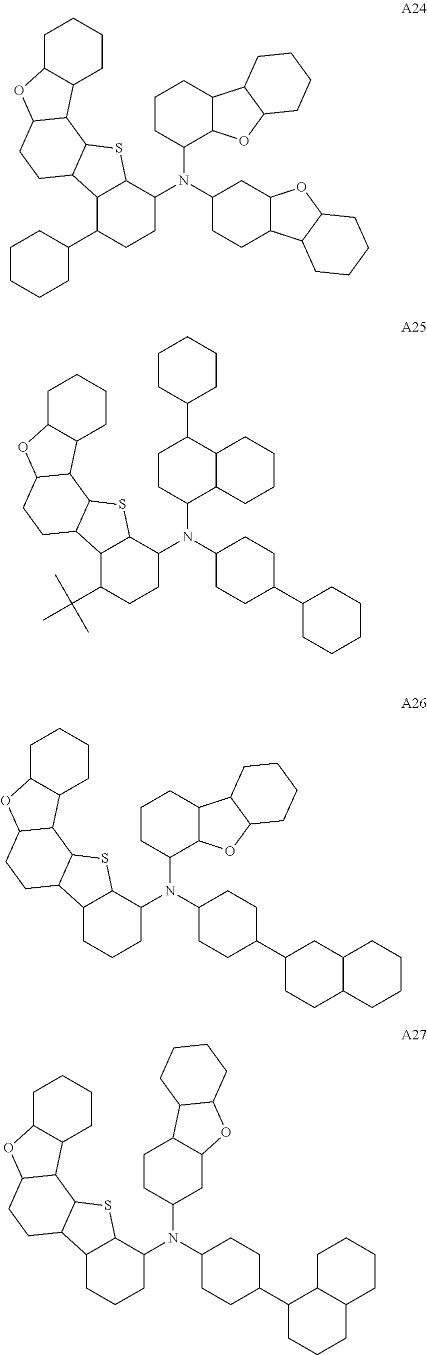 [SMILES: C1CCC(C2CCC(N(C3CCC4C(C3)OC3CCCCC34)C3CCCC4C5CCCCC5OC43)C3SC4C(CCC5OC6CCCCC6C54)C23)CC1 ; CC(C)(C)C1CCC(N(C2CCC(C3CCCCC3)CC2)C2CCC(C3CCCCC3)C3CCCCC32)C2SC3C(CCC4OC5CCCCC5C43)C21 ; C1CCC2CC(C3CCC(N(C4CCCC5C6CCCCC6OC54)C4CCCC5C6CCC7OC8CCCCC8C7C6SC54)CC3)CCC2C1 ; C1CCC2C(C1)CCCC2C1CCC(N(C2CCC3C(C2)OC2CCCCC23)C2CCCC3C4CCC5OC6CCCCC6C5C4SC32)CC1]

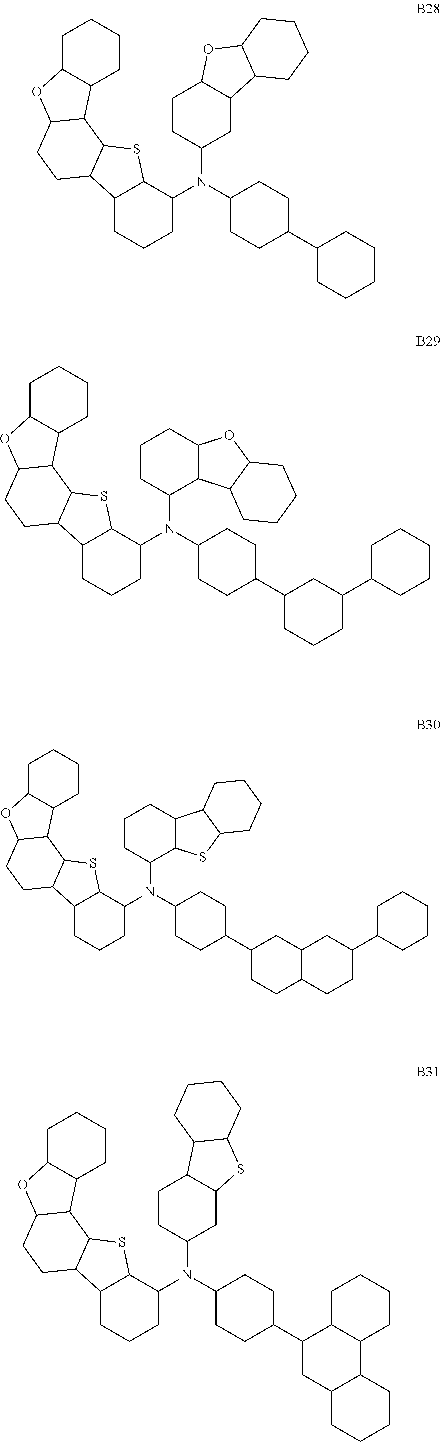 [SMILES: C1CCC(C2CCC(N(C3CCC4OC5CCCCC5C4C3)C3CCCC4C5CCC6OC7CCCCC7C6C5SC43)CC2)CC1 ; C1CCC(C2CCCC(C3CCC(N(C4CCCC5C6CCC7OC8CCCCC8C7C6SC54)C4CCCC5OC6CCCCC6C54)CC3)C2)CC1 ; C1CCC(C2CCC3CCC(C4CCC(N(C5CCCC6C7CCCCC7SC65)C5CCCC6C7CCC8OC9CCCCC9C8C7SC65)CC4)CC3C2)CC1 ; C1CCC2C(C1)CC(C1CCC(N(C3CCC4C(C3)SC3CCCCC34)C3CCCC4C5CCC6OC7CCCCC7C6C5SC43)CC1)C1CCCCC21]

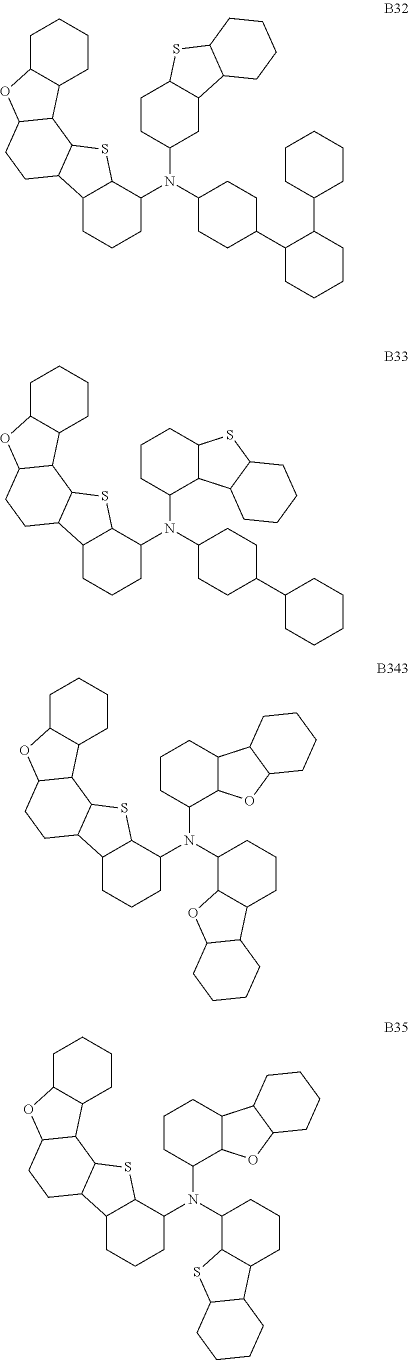 [SMILES: C1CCC(C2CCCCC2C2CCC(N(C3CCC4SC5CCCCC5C4C3)C3CCCC4C5CCC6OC7CCCCC7C6C5SC43)CC2)CC1 ; C1CCC(C2CCC(N(C3CCCC4C5CCC6OC7CCCCC7C6C5SC43)C3CCCC4SC5CCCCC5C43)CC2)CC1 ; C1CCC2C(C1)OC1C2CCCC1N(C1CCCC2C3CCCCC3OC21)C1CCCC2C3CCC4OC5CCCCC5C4C3SC21 ; C1CCC2C(C1)OC1C2CCCC1N(C1CCCC2C3CCCCC3SC21)C1CCCC2C3CCC4OC5CCCCC5C4C3SC21]

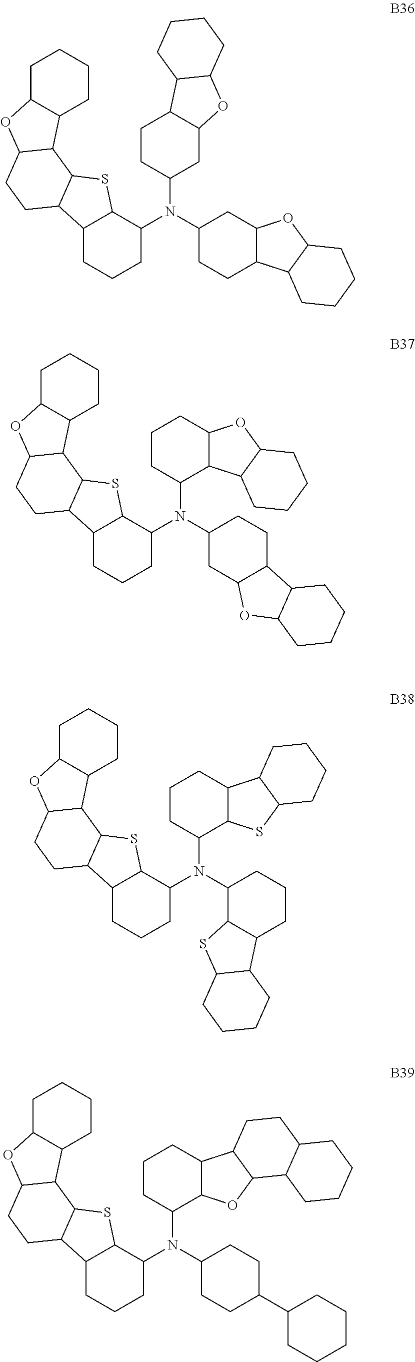 [SMILES: C1CCC2C(C1)OC1CC(N(C3CCC4C(C3)OC3CCCCC34)C3CCCC4C5CCC6OC7CCCCC7C6C5SC43)CCC12 ; C1CCC2C(C1)OC1CC(N(C3CCCC4C5CCC6OC7CCCCC7C6C5SC43)C3CCCC4OC5CCCCC5C43)CCC12 ; C1CCC2C(C1)OC1CCC3C4CCCC(N(C5CCCC6C7CCCCC7SC65)C5CCCC6C7CCCCC7SC65)C4SC3C12 ; C1CCC(C2CCC(N(C3CCCC4C5CCC6CCCCC6C5OC43)C3CCCC4C5CCC6OC7CCCCC7C6C5SC43)CC2)CC1]

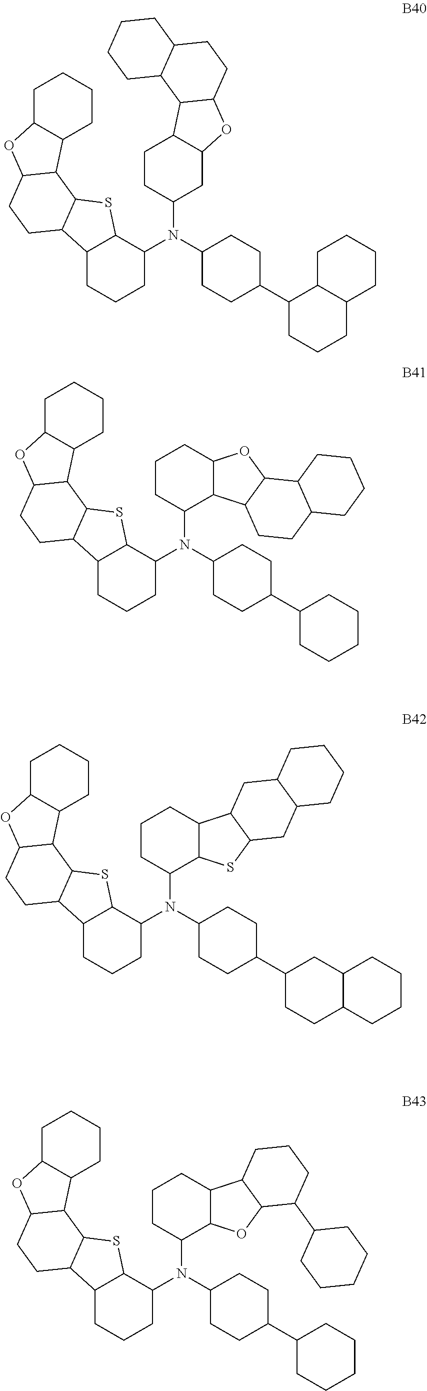 [SMILES: C1CCC2C(C1)CCCC2C1CCC(N(C2CCC3C(C2)OC2CCC4CCCCC4C23)C2CCCC3C4CCC5OC6CCCCC6C5C4SC32)CC1 ; C1CCC(C2CCC(N(C3CCCC4C5CCC6OC7CCCCC7C6C5SC43)C3CCCC4OC5C6CCCCC6CCC5C43)CC2)CC1 ; C1CCC2CC(C3CCC(N(C4CCCC5C6CC7CCCCC7CC6SC54)C4CCCC5C6CCC7OC8CCCCC8C7C6SC54)CC3)CCC2C1 ; C1CCC(C2CCC(N(C3CCCC4C5CCCC(C6CCCCC6)C5OC43)C3CCCC4C5CCC6OC7CCCCC7C6C5SC43)CC2)CC1]

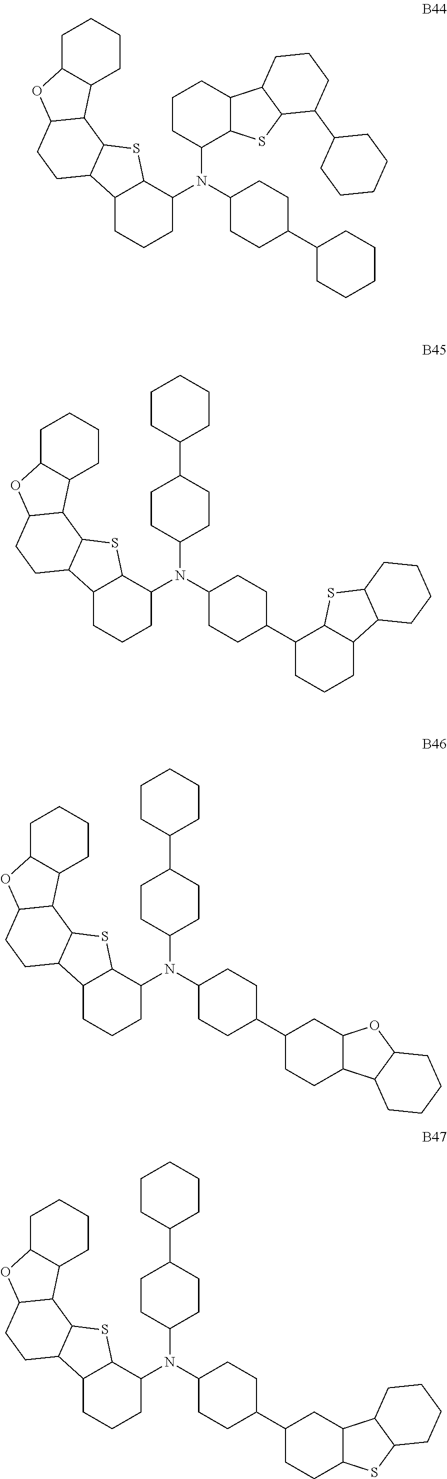 [SMILES: C1CCC(C2CCC(N(C3CCCC4C5CCCC(C6CCCCC6)C5SC43)C3CCCC4C5CCC6OC7CCCCC7C6C5SC43)CC2)CC1 ; C1CCC(C2CCC(N(C3CCC(C4CCCC5C6CCCCC6SC45)CC3)C3CCCC4C5CCC6OC7CCCCC7C6C5SC43)CC2)CC1 ; C1CCC(C2CCC(N(C3CCC(C4CCC5C(C4)OC4CCCCC45)CC3)C3CCCC4C5CCC6OC7CCCCC7C6C5SC43)CC2)CC1 ; C1CCC(C2CCC(N(C3CCC(C4CCC5SC6CCCCC6C5C4)CC3)C3CCCC4C5CCC6OC7CCCCC7C6C5SC43)CC2)CC1]

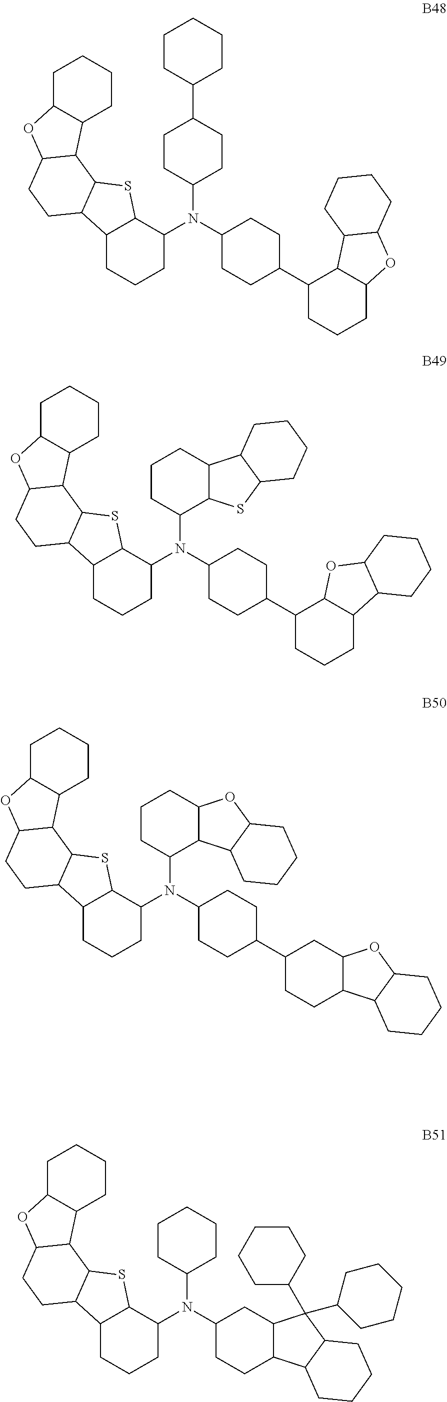 [SMILES: C1CCC(C2CCC(N(C3CCC(C4CCCC5OC6CCCCC6C54)CC3)C3CCCC4C5CCC6OC7CCCCC7C6C5SC43)CC2)CC1 ; C1CCC2C(C1)OC1C(C3CCC(N(C4CCCC5C6CCCCC6SC54)C4CCCC5C6CCC7OC8CCCCC8C7C6SC54)CC3)CCCC21 ; C1CCC2C(C1)OC1CC(C3CCC(N(C4CCCC5C6CCC7OC8CCCCC8C7C6SC54)C4CCCC5OC6CCCCC6C54)CC3)CCC12 ; C1CCC(N(C2CCC3C4CCCCC4C(C4CCCCC4)(C4CCCCC4)C3C2)C2CCCC3C4CCC5OC6CCCCC6C5C4SC32)CC1]

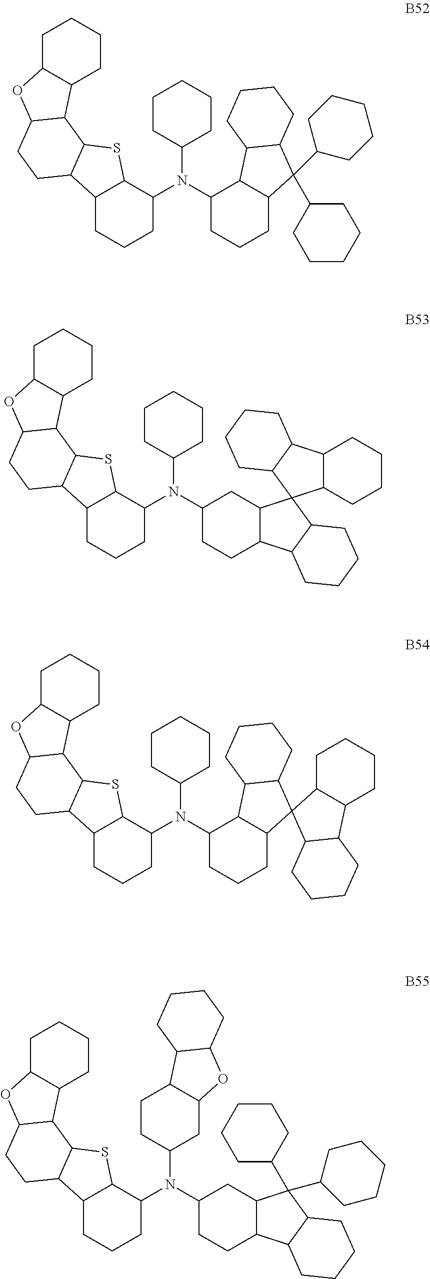 [SMILES: C1CCC(N(C2CCCC3C4CCC5OC6CCCCC6C5C4SC32)C2CCCC3C2C2CCCCC2C3(C2CCCCC2)C2CCCCC2)CC1 ; C1CCC(N(C2CCC3C4CCCCC4C4(C5CCCCC5C5CCCCC54)C3C2)C2CCCC3C4CCC5OC6CCCCC6C5C4SC32)CC1 ; C1CCC(N(C2CCCC3C4CCC5OC6CCCCC6C5C4SC32)C2CCCC3C2C2CCCCC2C32C3CCCCC3C3CCCCC32)CC1 ; C1CCC(C2(C3CCCCC3)C3CCCCC3C3CCC(N(C4CCC5C(C4)OC4CCCCC45)C4CCCC5C6CCC7OC8CCCCC8C7C6SC54)CC32)CC1]

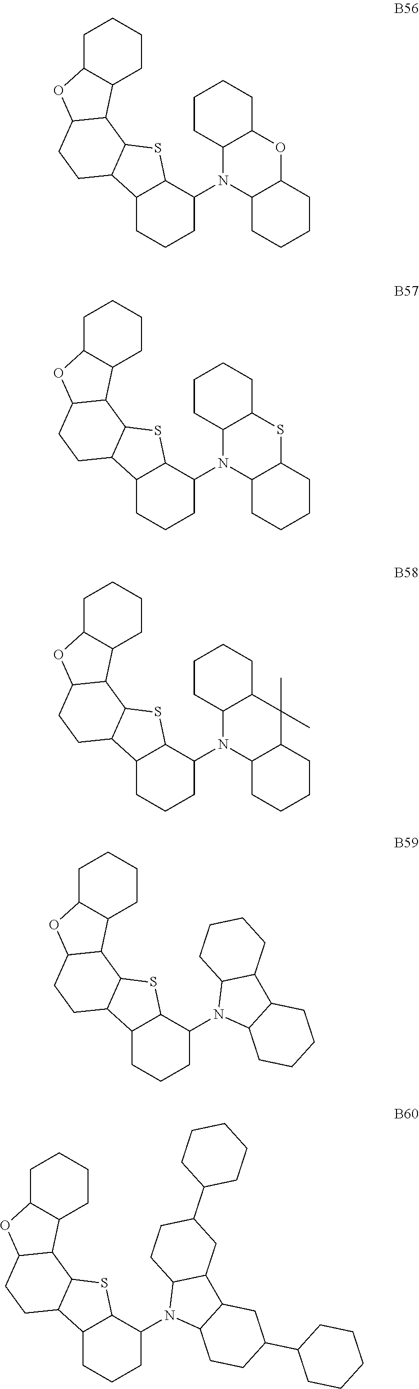 [SMILES: C1CCC2C(C1)OC1CCC3C4CCCC(N5C6CCCCC6OC6CCCCC65)C4SC3C12 ; C1CCC2C(C1)OC1CCC3C4CCCC(N5C6CCCCC6SC6CCCCC65)C4SC3C12 ; CC1(C)C2CCCCC2N(C2CCCC3C4CCC5OC6CCCCC6C5C4SC32)C2CCCCC21 ; C1CCC2C(C1)OC1CCC3C4CCCC(N5C6CCCCC6C6CCCCC65)C4SC3C12 ; C1CCC(C2CCC3C(C2)C2CC(C4CCCCC4)CCC2N3C2CCCC3C4CCC5OC6CCCCC6C5C4SC32)CC1]

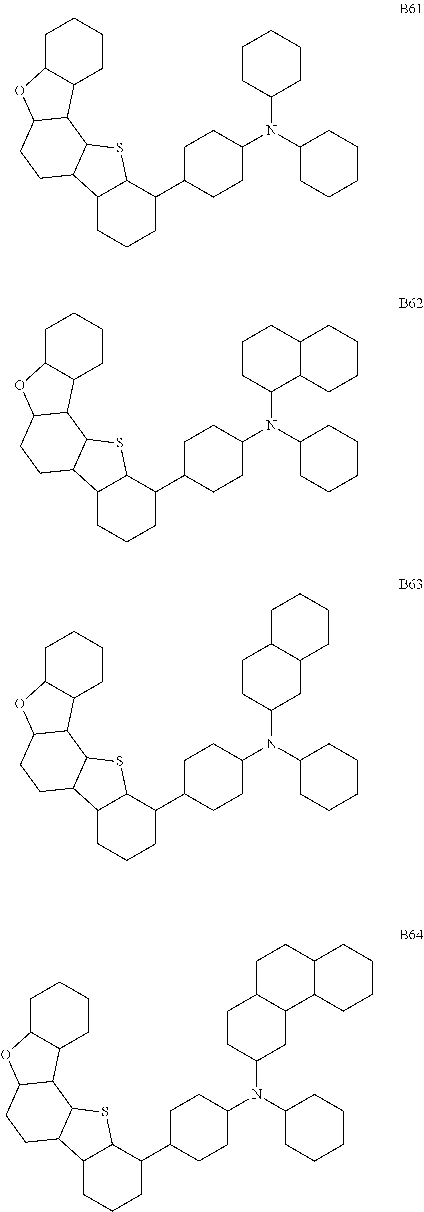 [SMILES: C1CCC(N(C2CCCCC2)C2CCC(C3CCCC4C5CCC6OC7CCCCC7C6C5SC34)CC2)CC1 ; C1CCC(N(C2CCC(C3CCCC4C5CCC6OC7CCCCC7C6C5SC34)CC2)C2CCCC3CCCCC32)CC1 ; C1CCC(N(C2CCC(C3CCCC4C5CCC6OC7CCCCC7C6C5SC34)CC2)C2CCC3CCCCC3C2)CC1 ; C1CCC(N(C2CCC(C3CCCC4C5CCC6OC7CCCCC7C6C5SC34)CC2)C2CCC3CCC4CCCCC4C3C2)CC1]

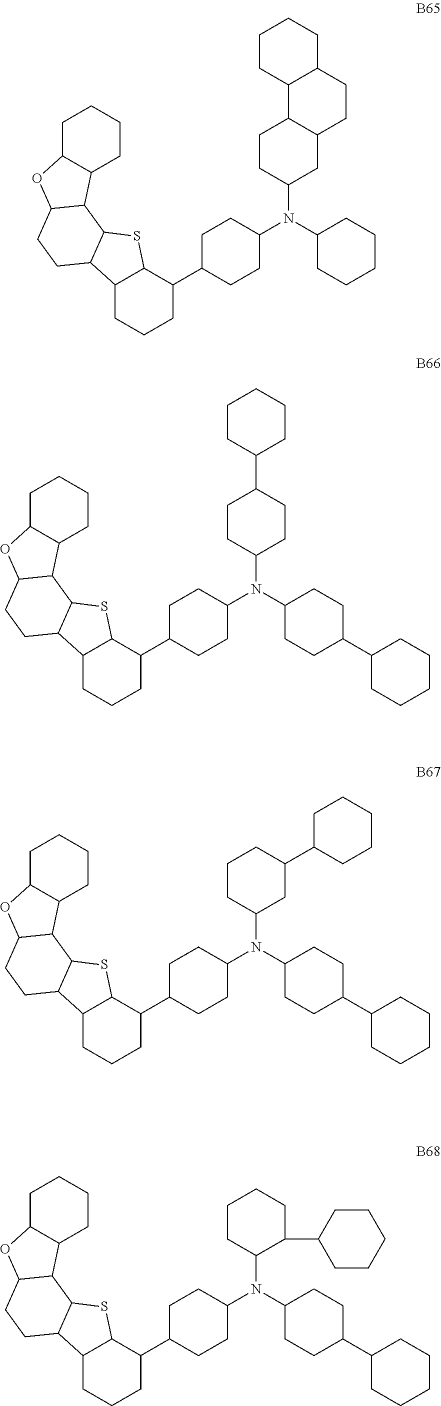 [SMILES: C1CCC(N(C2CCC(C3CCCC4C5CCC6OC7CCCCC7C6C5SC34)CC2)C2CCC3C(CCC4CCCCC43)C2)CC1 ; C1CCC(C2CCC(N(C3CCC(C4CCCCC4)CC3)C3CCC(C4CCCC5C6CCC7OC8CCCCC8C7C6SC45)CC3)CC2)CC1 ; C1CCC(C2CCC(N(C3CCC(C4CCCC5C6CCC7OC8CCCCC8C7C6SC45)CC3)C3CCCC(C4CCCCC4)C3)CC2)CC1 ; C1CCC(C2CCC(N(C3CCC(C4CCCC5C6CCC7OC8CCCCC8C7C6SC45)CC3)C3CCCCC3C3CCCCC3)CC2)CC1]

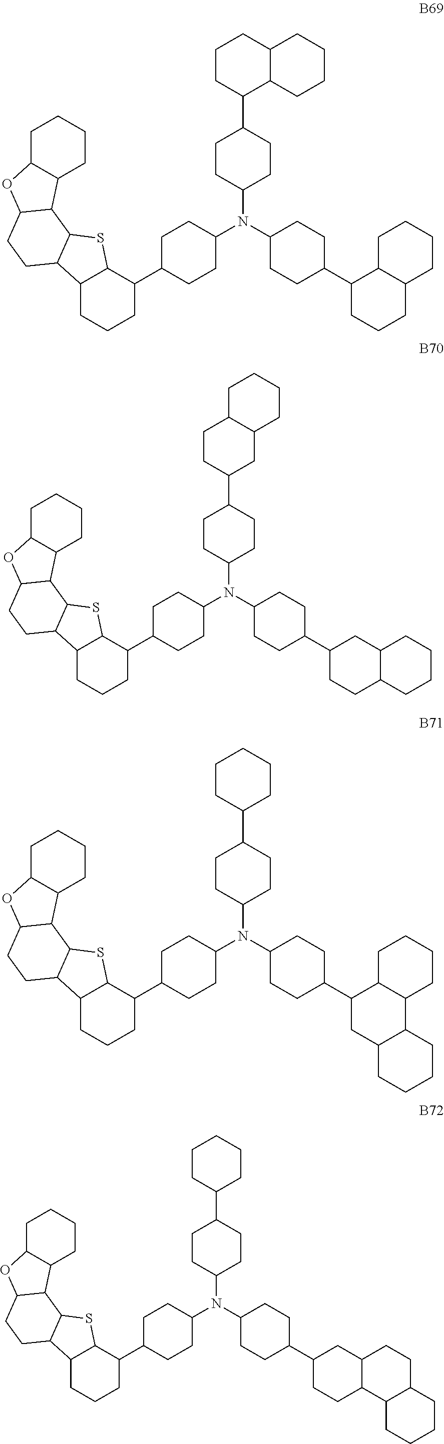 [SMILES: C1CCC2C(C1)CCCC2C1CCC(N(C2CCC(C3CCCC4CCCCC43)CC2)C2CCC(C3CCCC4C5CCC6OC7CCCCC7C6C5SC34)CC2)CC1 ; C1CCC2CC(C3CCC(N(C4CCC(C5CCC6CCCCC6C5)CC4)C4CCC(C5CCCC6C7CCC8OC9CCCCC9C8C7SC56)CC4)CC3)CCC2C1 ; C1CCC(C2CCC(N(C3CCC(C4CC5CCCCC5C5CCCCC45)CC3)C3CCC(C4CCCC5C6CCC7OC8CCCCC8C7C6SC45)CC3)CC2)CC1 ; C1CCC(C2CCC(N(C3CCC(C4CCC5C(CCC6CCCCC65)C4)CC3)C3CCC(C4CCCC5C6CCC7OC8CCCCC8C7C6SC45)CC3)CC2)CC1]

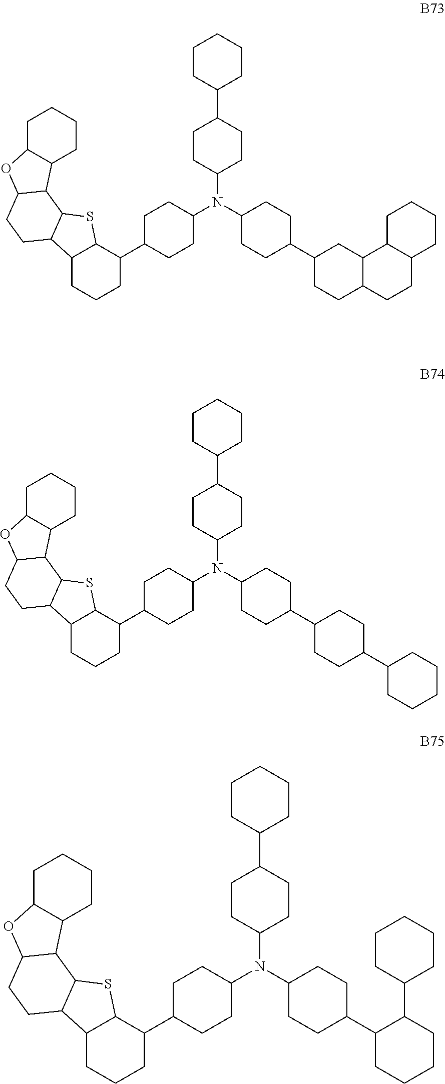 [SMILES: C1CCC(C2CCC(N(C3CCC(C4CCC5CCC6CCCCC6C5C4)CC3)C3CCC(C4CCCC5C6CCC7OC8CCCCC8C7C6SC45)CC3)CC2)CC1 ; C1CCC(C2CCC(C3CCC(N(C4CCC(C5CCCCC5)CC4)C4CCC(C5CCCC6C7CCC8OC9CCCCC9C8C7SC56)CC4)CC3)CC2)CC1 ; C1CCC(C2CCC(N(C3CCC(C4CCCCC4C4CCCCC4)CC3)C3CCC(C4CCCC5C6CCC7OC8CCCCC8C7C6SC45)CC3)CC2)CC1]

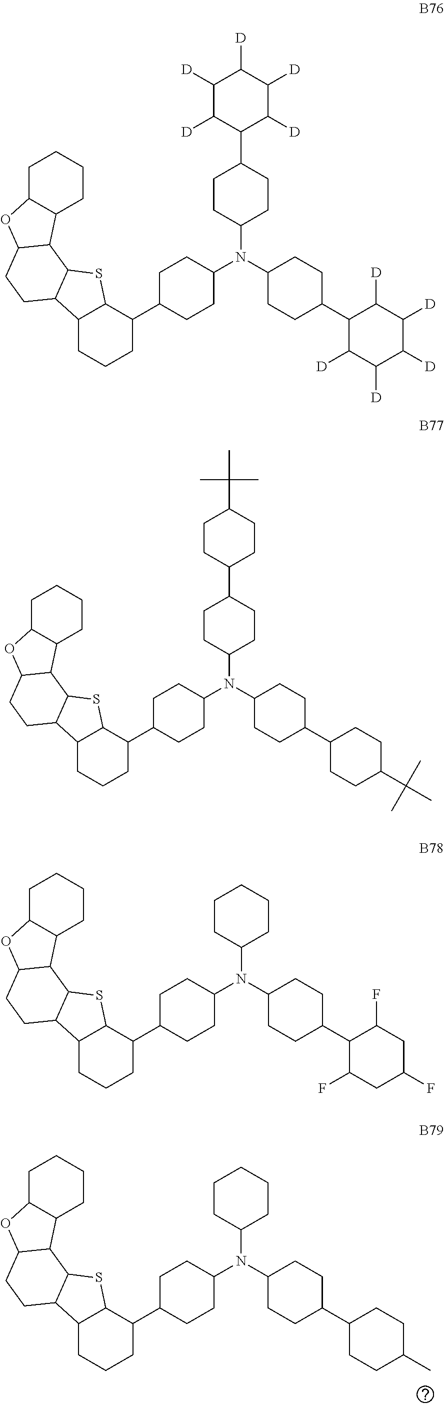 [SMILES: [2H]C1C([2H])C([2H])C(C2CCC(N(C3CCC(C4CCCC5C6CCC7OC8CCCCC8C7C6SC45)CC3)C3CCC(C4C([2H])C([2H])C([2H])C([2H])C4[2H])CC3)CC2)C([2H])C1[2H] ; CC(C)(C)C1CCC(C2CCC(N(C3CCC(C4CCC(C(C)(C)C)CC4)CC3)C3CCC(C4CCCC5C6CCC7OC8CCCCC8C7C6SC45)CC3)CC2)CC1 ; FC1CC(F)C(C2CCC(N(C3CCCCC3)C3CCC(C4CCCC5C6CCC7OC8CCCCC8C7C6SC45)CC3)CC2)C(F)C1 ; CC1CCC(C2CCC(N(C3CCCCC3)C3CCC(C4CCCC5C6CCC7OC8CCCCC8C7C6SC45)CC3)CC2)CC1]

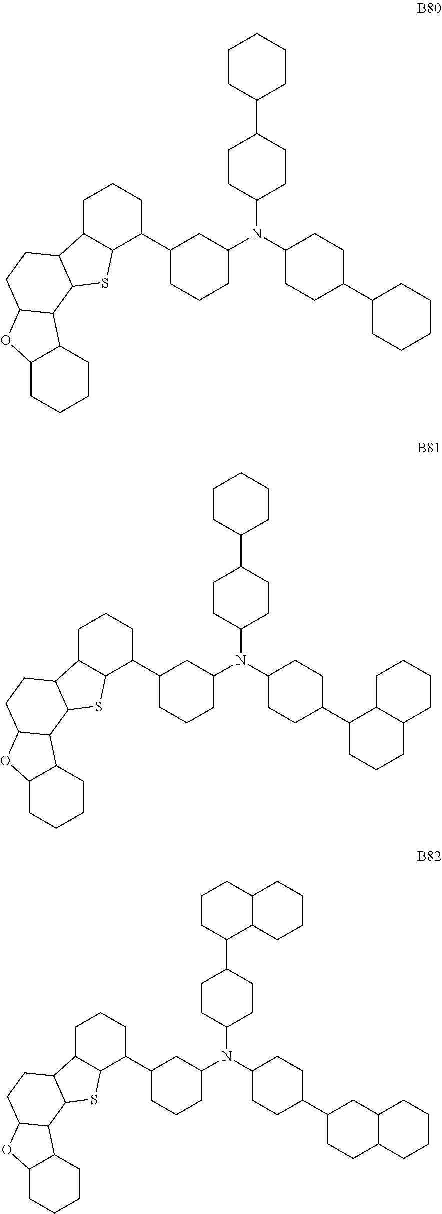 [SMILES: C1CCC(C2CCC(N(C3CCC(C4CCCCC4)CC3)C3CCCC(C4CCCC5C6CCC7OC8CCCCC8C7C6SC45)C3)CC2)CC1 ; C1CCC(C2CCC(N(C3CCC(C4CCCC5CCCCC54)CC3)C3CCCC(C4CCCC5C6CCC7OC8CCCCC8C7C6SC45)C3)CC2)CC1 ; C1CCC2CC(C3CCC(N(C4CCC(C5CCCC6CCCCC65)CC4)C4CCCC(C5CCCC6C7CCC8OC9CCCCC9C8C7SC56)C4)CC3)CCC2C1]

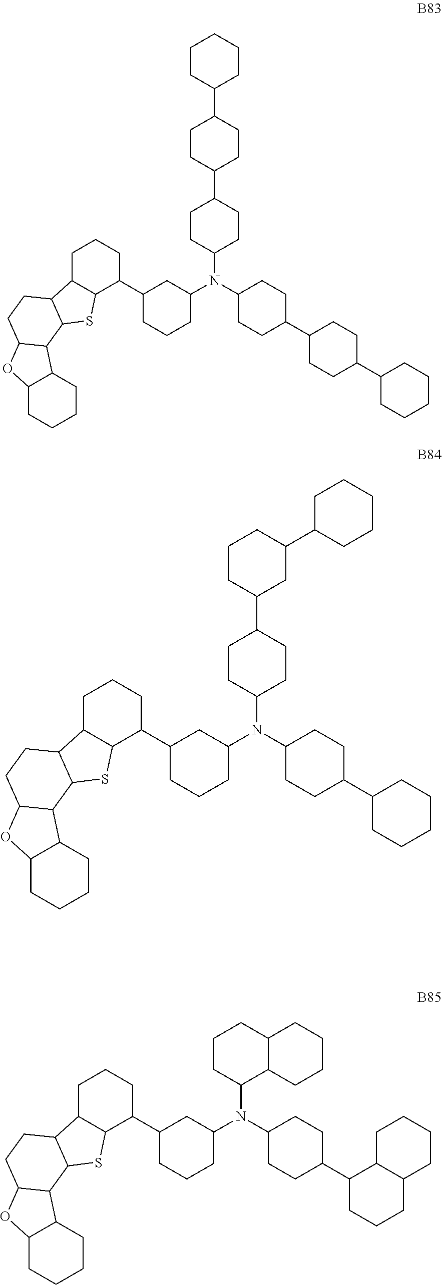 [SMILES: C1CCC(C2CCC(C3CCC(N(C4CCC(C5CCC(C6CCCCC6)CC5)CC4)C4CCCC(C5CCCC6C7CCC8OC9CCCCC9C8C7SC56)C4)CC3)CC2)CC1 ; C1CCC(C2CCC(N(C3CCC(C4CCCC(C5CCCCC5)C4)CC3)C3CCCC(C4CCCC5C6CCC7OC8CCCCC8C7C6SC45)C3)CC2)CC1 ; C1CCC2C(C1)CCCC2C1CCC(N(C2CCCC(C3CCCC4C5CCC6OC7CCCCC7C6C5SC34)C2)C2CCCC3CCCCC32)CC1]

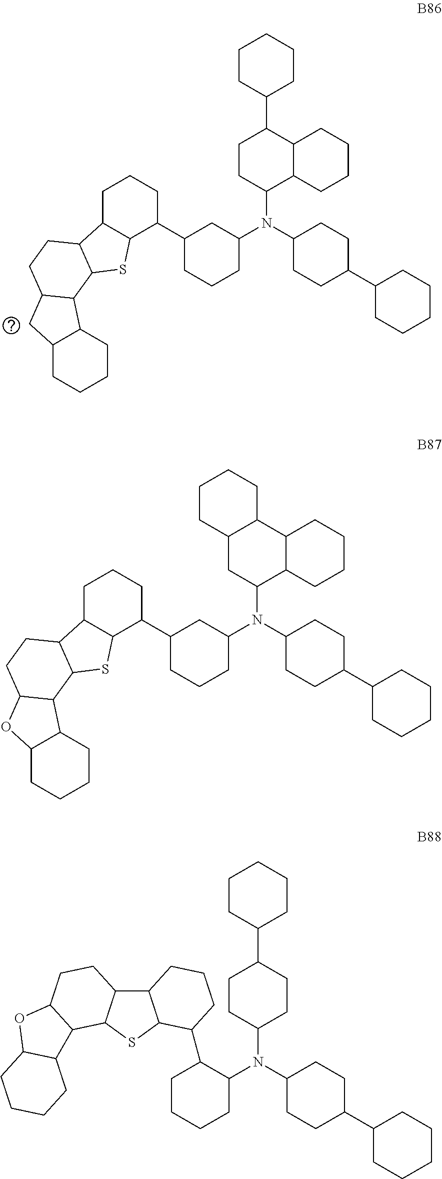 [SMILES: C1CCC(C2CCC(N(C3CCCC(C4CCCC5C6CCC7CC8CCCCC8C7C6SC45)C3)C3CCC(C4CCCCC4)C4CCCCC43)CC2)CC1 ; C1CCC(C2CCC(N(C3CCCC(C4CCCC5C6CCC7OC8CCCCC8C7C6SC45)C3)C3CC4CCCCC4C4CCCCC43)CC2)CC1 ; C1CCC(C2CCC(N(C3CCC(C4CCCCC4)CC3)C3CCCCC3C3CCCC4C5CCC6OC7CCCCC7C6C5SC43)CC2)CC1]

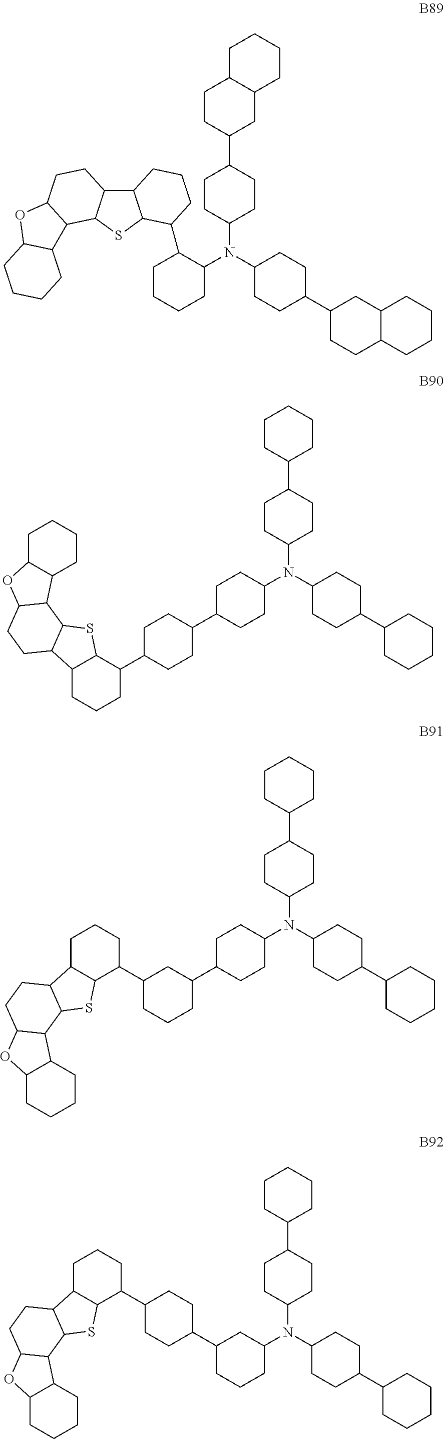 [SMILES: C1CCC2CC(C3CCC(N(C4CCC(C5CCC6CCCCC6C5)CC4)C4CCCCC4C4CCCC5C6CCC7OC8CCCCC8C7C6SC54)CC3)CCC2C1 ; C1CCC(C2CCC(N(C3CCC(C4CCCCC4)CC3)C3CCC(C4CCC(C5CCCC6C7CCC8OC9CCCCC9C8C7SC56)CC4)CC3)CC2)CC1 ; C1CCC(C2CCC(N(C3CCC(C4CCCCC4)CC3)C3CCC(C4CCCC(C5CCCC6C7CCC8OC9CCCCC9C8C7SC56)C4)CC3)CC2)CC1 ; C1CCC(C2CCC(N(C3CCC(C4CCCCC4)CC3)C3CCCC(C4CCC(C5CCCC6C7CCC8OC9CCCCC9C8C7SC56)CC4)C3)CC2)CC1]

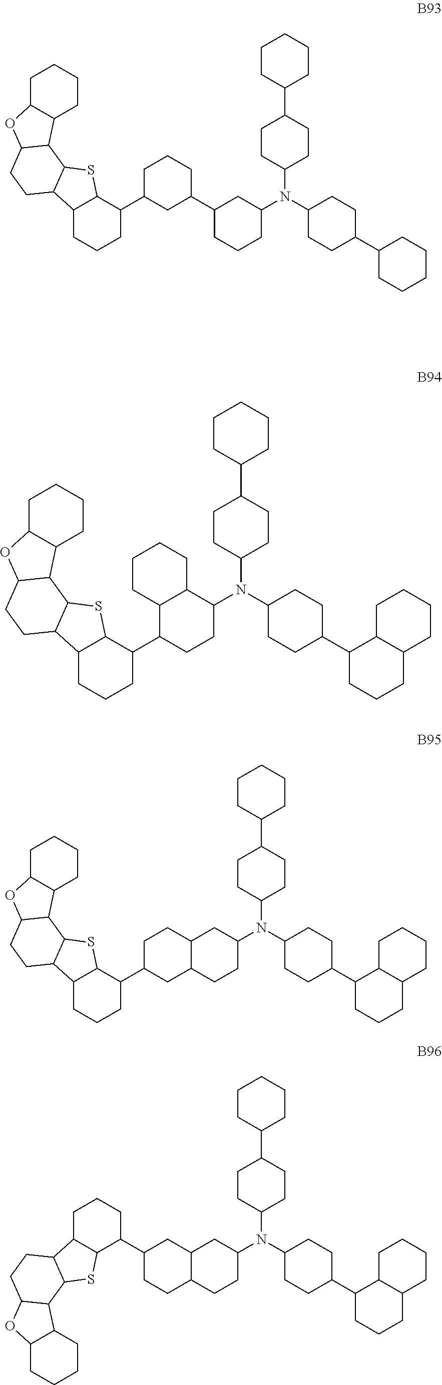 [SMILES: C1CCC(C2CCC(N(C3CCC(C4CCCCC4)CC3)C3CCCC(C4CCCC(C5CCCC6C7CCC8OC9CCCCC9C8C7SC56)C4)C3)CC2)CC1 ; C1CCC(C2CCC(N(C3CCC(C4CCCC5CCCCC54)CC3)C3CCC(C4CCCC5C6CCC7OC8CCCCC8C7C6SC45)C4CCCCC43)CC2)CC1 ; C1CCC(C2CCC(N(C3CCC(C4CCCC5CCCCC54)CC3)C3CCC4CC(C5CCCC6C7CCC8OC9CCCCC9C8C7SC56)CCC4C3)CC2)CC1 ; C1CCC(C2CCC(N(C3CCC(C4CCCC5CCCCC54)CC3)C3CCC4CCC(C5CCCC6C7CCC8OC9CCCCC9C8C7SC56)CC4C3)CC2)CC1]

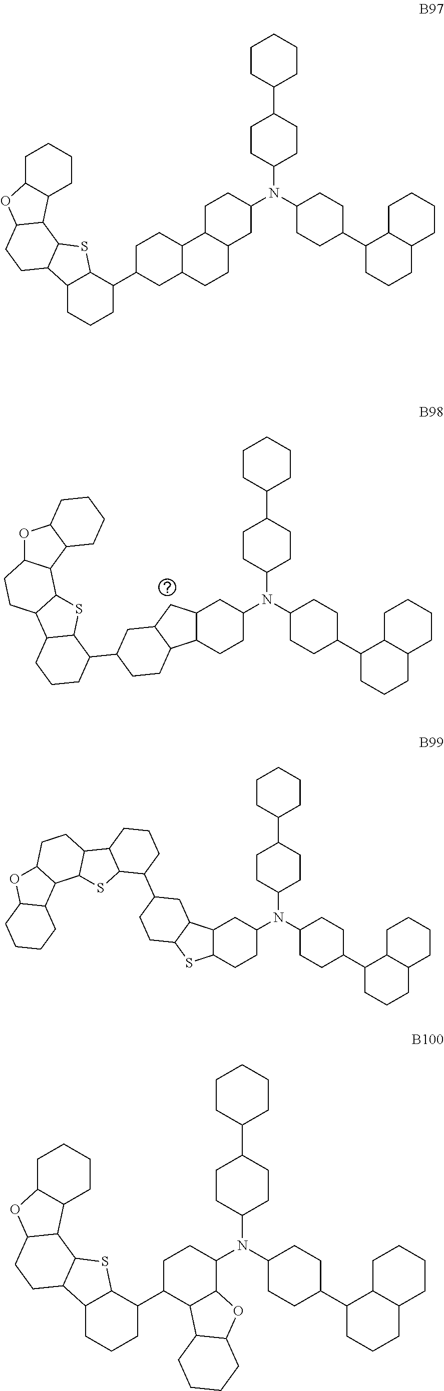 [SMILES: C1CCC(C2CCC(N(C3CCC(C4CCCC5CCCCC54)CC3)C3CCC4C(CCC5CC(C6CCCC7C8CCC9OC%10CCCCC%10C9C8SC67)CCC54)C3)CC2)CC1 ; C1CCC(C2CCC(N(C3CCC(C4CCCC5CCCCC54)CC3)C3CCC4C(CC5CC(C6CCCC7C8CCC9OC%10CCCCC%10C9C8SC67)CCC54)C3)CC2)CC1 ; C1CCC(C2CCC(N(C3CCC(C4CCCC5CCCCC54)CC3)C3CCC4SC5CCC(C6CCCC7C8CCC9OC%10CCCCC%10C9C8SC67)CC5C4C3)CC2)CC1 ; C1CCC(C2CCC(N(C3CCC(C4CCCC5CCCCC54)CC3)C3CCC(C4CCCC5C6CCC7OC8CCCCC8C7C6SC54)C4C5CCCCC5OC43)CC2)CC1]

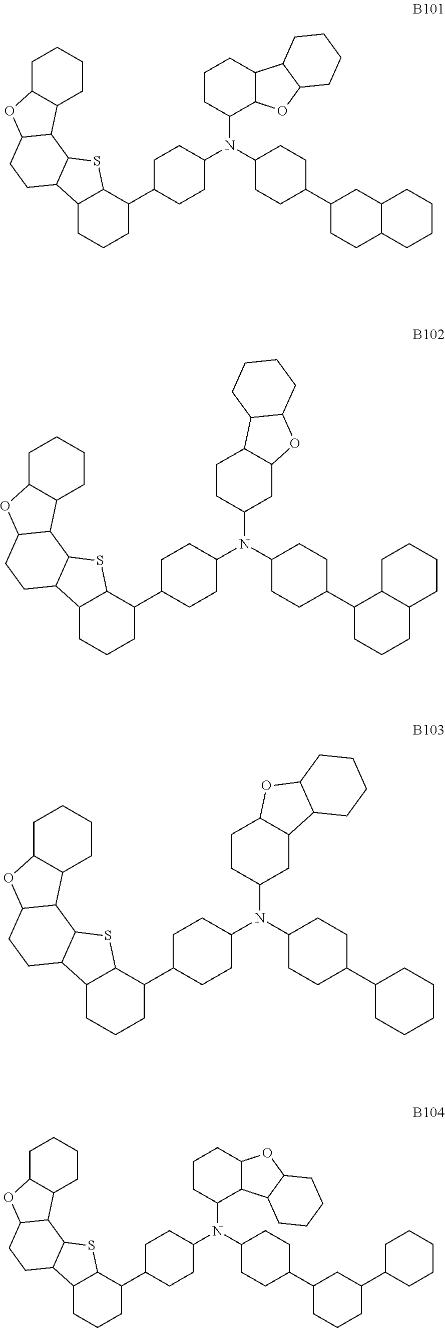 [SMILES: C1CCC2CC(C3CCC(N(C4CCC(C5CCCC6C7CCC8OC9CCCCC9C8C7SC56)CC4)C4CCCC5C6CCCCC6OC54)CC3)CCC2C1 ; C1CCC2C(C1)CCCC2C1CCC(N(C2CCC(C3CCCC4C5CCC6OC7CCCCC7C6C5SC34)CC2)C2CCC3C(C2)OC2CCCCC23)CC1 ; C1CCC(C2CCC(N(C3CCC(C4CCCC5C6CCC7OC8CCCCC8C7C6SC45)CC3)C3CCC4OC5CCCCC5C4C3)CC2)CC1 ; C1CCC(C2CCCC(C3CCC(N(C4CCC(C5CCCC6C7CCC8OC9CCCCC9C8C7SC56)CC4)C4CCCC5OC6CCCCC6C54)CC3)C2)CC1]

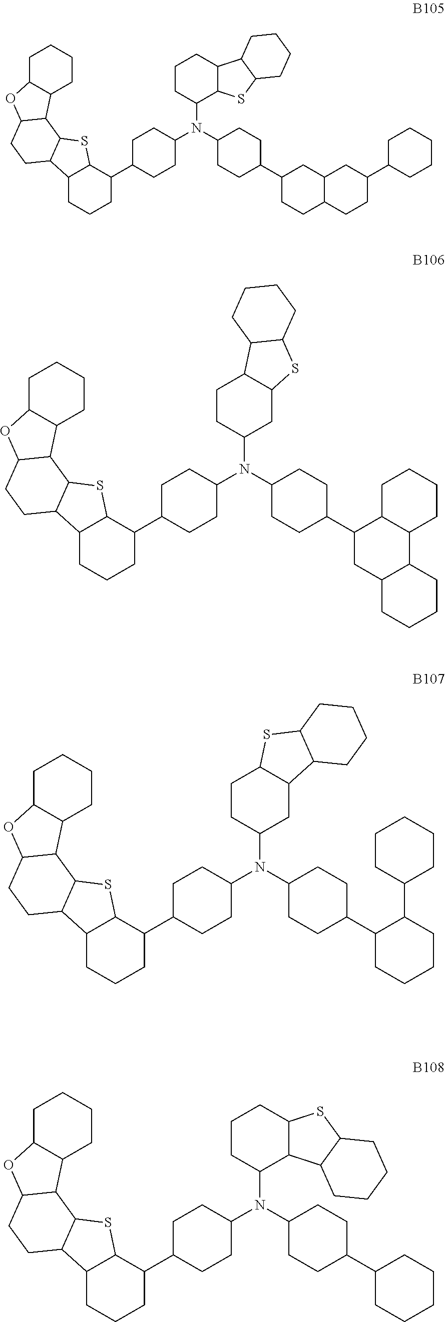 [SMILES: C1CCC(C2CCC3CCC(C4CCC(N(C5CCC(C6CCCC7C8CCC9OC%10CCCCC%10C9C8SC67)CC5)C5CCCC6C7CCCCC7SC65)CC4)CC3C2)CC1 ; C1CCC2C(C1)CC(C1CCC(N(C3CCC(C4CCCC5C6CCC7OC8CCCCC8C7C6SC45)CC3)C3CCC4C(C3)SC3CCCCC34)CC1)C1CCCCC21 ; C1CCC(C2CCCCC2C2CCC(N(C3CCC(C4CCCC5C6CCC7OC8CCCCC8C7C6SC45)CC3)C3CCC4SC5CCCCC5C4C3)CC2)CC1 ; C1CCC(C2CCC(N(C3CCC(C4CCCC5C6CCC7OC8CCCCC8C7C6SC45)CC3)C3CCCC4SC5CCCCC5C43)CC2)CC1]

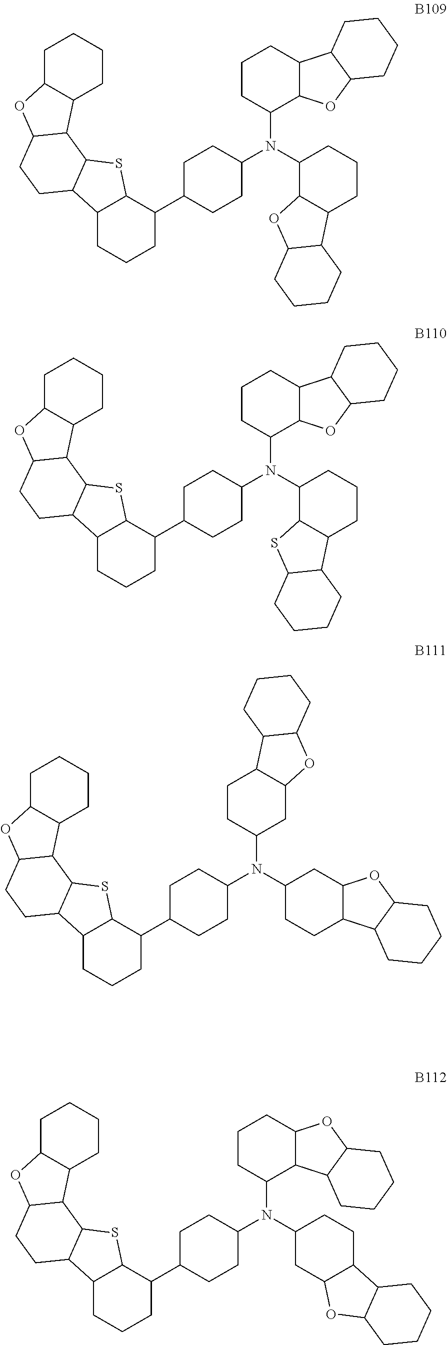

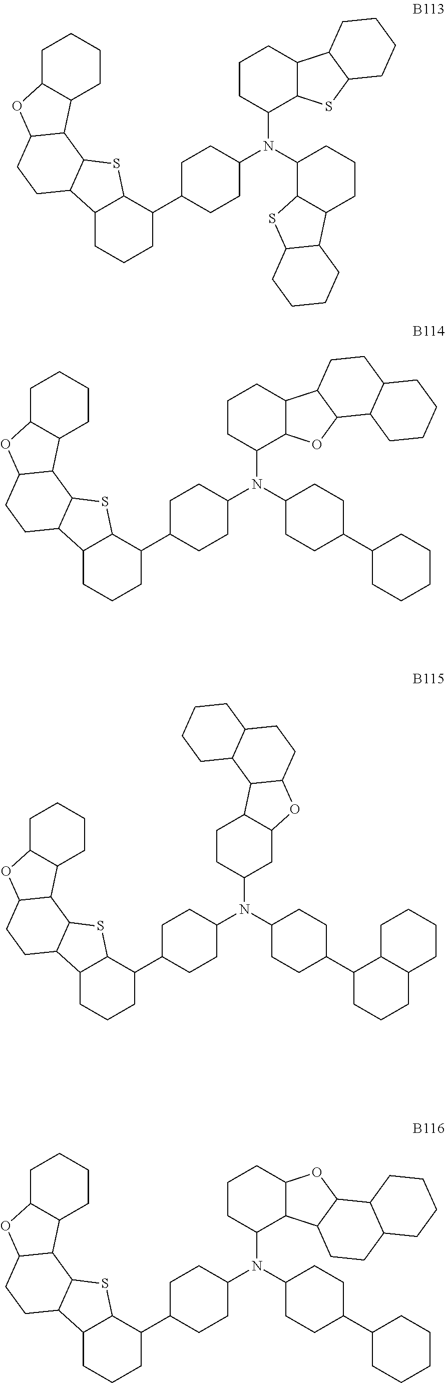 [SMILES: C1CCC2C(C1)OC1CCC3C4CCCC(C5CCC(N(C6CCCC7C8CCCCC8SC76)C6CCCC7C8CCCCC8SC76)CC5)C4SC3C12 ; C1CCC(C2CCC(N(C3CCC(C4CCCC5C6CCC7OC8CCCCC8C7C6SC45)CC3)C3CCCC4C5CCC6CCCCC6C5OC43)CC2)CC1 ; C1CCC2C(C1)CCCC2C1CCC(N(C2CCC(C3CCCC4C5CCC6OC7CCCCC7C6C5SC34)CC2)C2CCC3C(C2)OC2CCC4CCCCC4C23)CC1 ; C1CCC(C2CCC(N(C3CCC(C4CCCC5C6CCC7OC8CCCCC8C7C6SC45)CC3)C3CCCC4OC5C6CCCCC6CCC5C43)CC2)CC1]

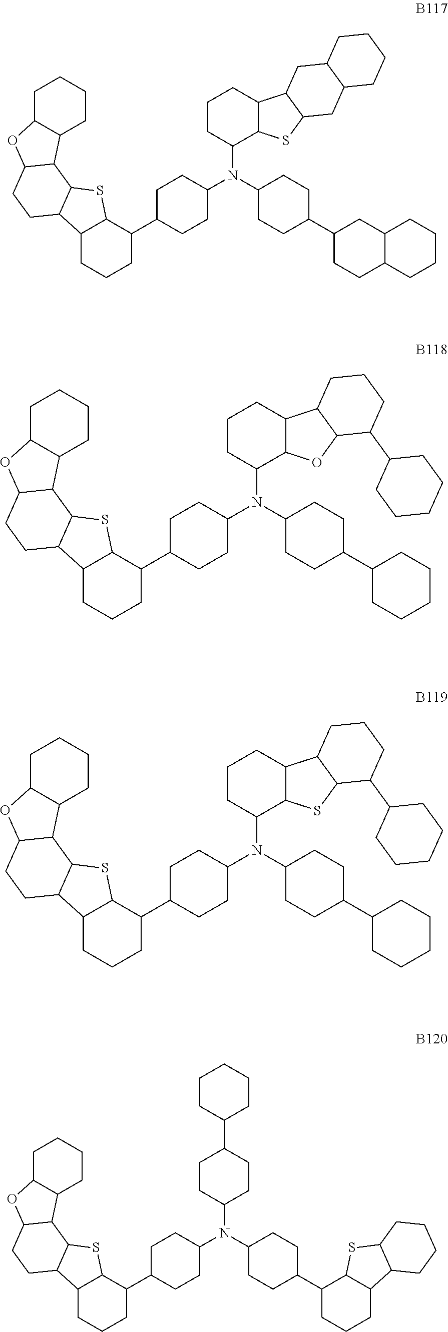 [SMILES: C1CCC2CC(C3CCC(N(C4CCC(C5CCCC6C7CCC8OC9CCCCC9C8C7SC56)CC4)C4CCCC5C6CC7CCCCC7CC6SC54)CC3)CCC2C1 ; C1CCC(C2CCC(N(C3CCC(C4CCCC5C6CCC7OC8CCCCC8C7C6SC45)CC3)C3CCCC4C5CCCC(C6CCCCC6)C5OC43)CC2)CC1 ; C1CCC(C2CCC(N(C3CCC(C4CCCC5C6CCC7OC8CCCCC8C7C6SC45)CC3)C3CCCC4C5CCCC(C6CCCCC6)C5SC43)CC2)CC1 ; C1CCC(C2CCC(N(C3CCC(C4CCCC5C6CCCCC6SC45)CC3)C3CCC(C4CCCC5C6CCC7OC8CCCCC8C7C6SC45)CC3)CC2)CC1]

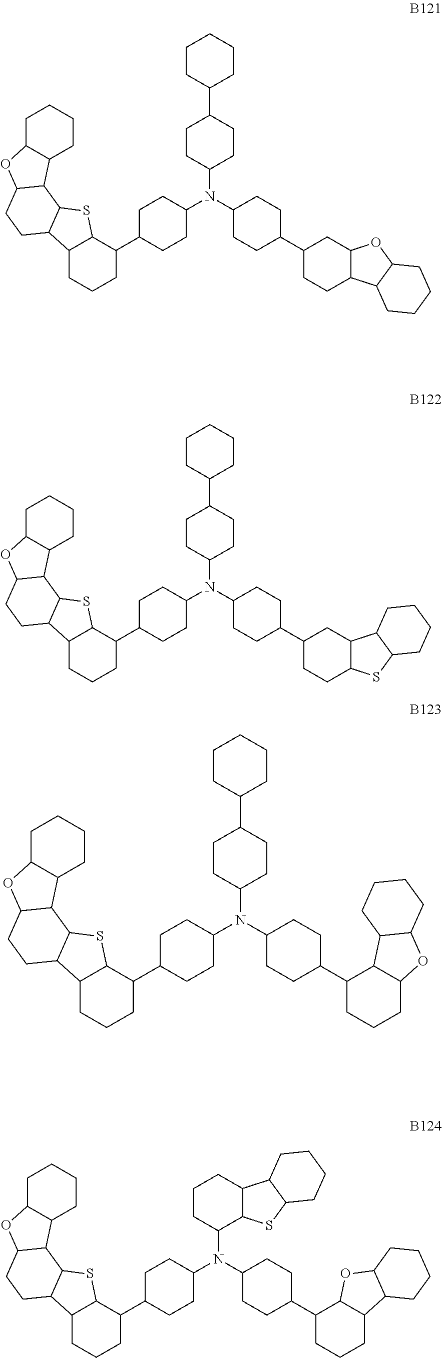 [SMILES: C1CCC(C2CCC(N(C3CCC(C4CCC5C(C4)OC4CCCCC45)CC3)C3CCC(C4CCCC5C6CCC7OC8CCCCC8C7C6SC45)CC3)CC2)CC1 ; C1CCC(C2CCC(N(C3CCC(C4CCC5SC6CCCCC6C5C4)CC3)C3CCC(C4CCCC5C6CCC7OC8CCCCC8C7C6SC45)CC3)CC2)CC1 ; C1CCC(C2CCC(N(C3CCC(C4CCCC5C6CCC7OC8CCCCC8C7C6SC45)CC3)C3CCC(C4CCCC5OC6CCCCC6C54)CC3)CC2)CC1 ; C1CCC2C(C1)OC1C(C3CCC(N(C4CCC(C5CCCC6C7CCC8OC9CCCCC9C8C7SC56)CC4)C4CCCC5C6CCCCC6SC54)CC3)CCCC21]

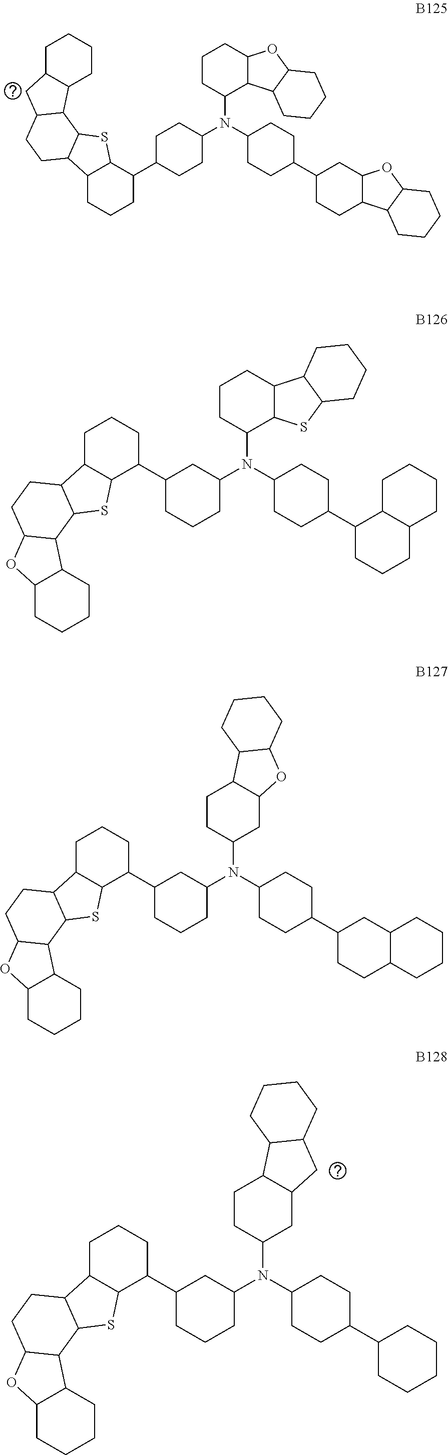 [SMILES: C1CCC2C(C1)CC1CCC3C4CCCC(C5CCC(N(C6CCC(C7CCC8C(C7)OC7CCCCC78)CC6)C6CCCC7OC8CCCCC8C76)CC5)C4SC3C12 ; C1CCC2C(C1)CCCC2C1CCC(N(C2CCCC(C3CCCC4C5CCC6OC7CCCCC7C6C5SC34)C2)C2CCCC3C4CCCCC4SC32)CC1 ; C1CCC2CC(C3CCC(N(C4CCCC(C5CCCC6C7CCC8OC9CCCCC9C8C7SC56)C4)C4CCC5C(C4)OC4CCCCC45)CC3)CCC2C1 ; C1CCC(C2CCC(N(C3CCC4C(CC5CCCCC54)C3)C3CCCC(C4CCCC5C6CCC7OC8CCCCC8C7C6SC45)C3)CC2)CC1]

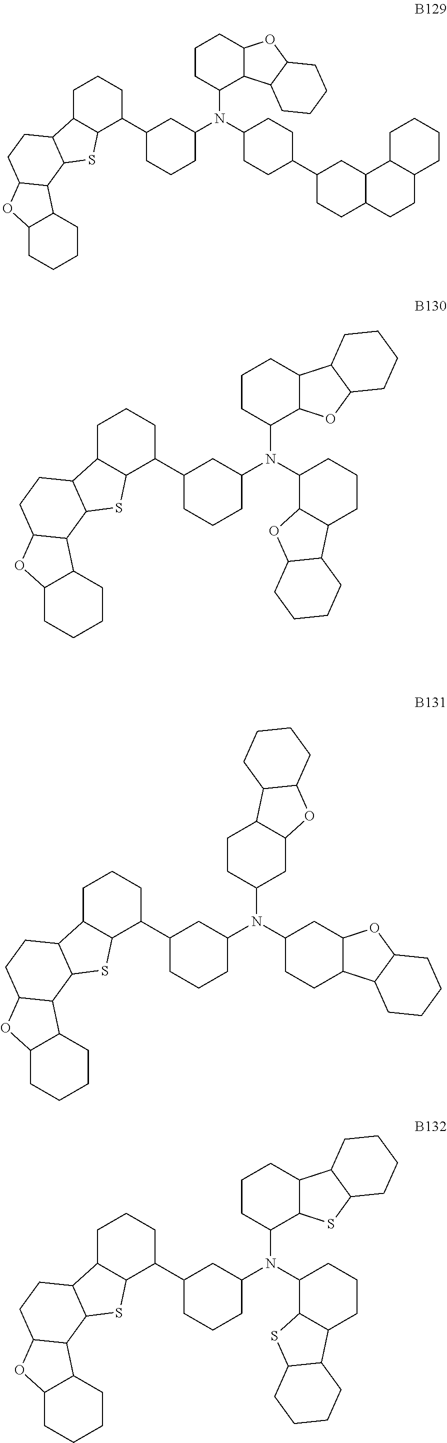 [SMILES: C1CCC2C(C1)CCC1CCC(C3CCC(N(C4CCCC(C5CCCC6C7CCC8OC9CCCCC9C8C7SC56)C4)C4CCCC5OC6CCCCC6C54)CC3)CC12 ; C1CCC2C(C1)OC1C2CCCC1N(C1CCCC(C2CCCC3C4CCC5OC6CCCCC6C5C4SC23)C1)C1CCCC2C3CCCCC3OC21 ; C1CCC2C(C1)OC1CC(N(C3CCCC(C4CCCC5C6CCC7OC8CCCCC8C7C6SC45)C3)C3CCC4C(C3)OC3CCCCC34)CCC12 ; C1CCC2C(C1)OC1CCC3C4CCCC(C5CCCC(N(C6CCCC7C8CCCCC8SC76)C6CCCC7C8CCCCC8SC76)C5)C4SC3C12]

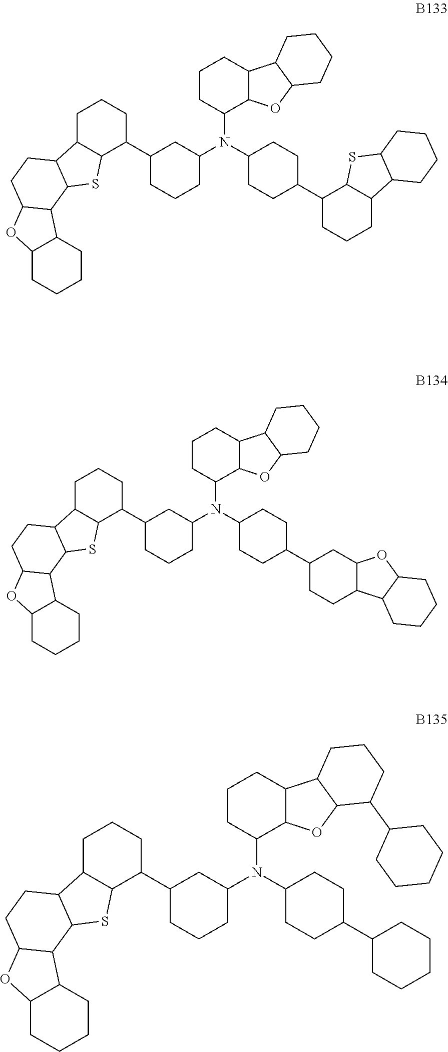 [SMILES: C1CCC2C(C1)OC1C2CCCC1N(C1CCC(C2CCCC3C4CCCCC4SC23)CC1)C1CCCC(C2CCCC3C4CCC5OC6CCCCC6C5C4SC23)C1 ; C1CCC2C(C1)OC1CC(C3CCC(N(C4CCCC(C5CCCC6C7CCC8OC9CCCCC9C8C7SC56)C4)C4CCCC5C6CCCCC6OC54)CC3)CCC12 ; C1CCC(C2CCC(N(C3CCCC(C4CCCC5C6CCC7OC8CCCCC8C7C6SC45)C3)C3CCCC4C5CCCC(C6CCCCC6)C5OC43)CC2)CC1]

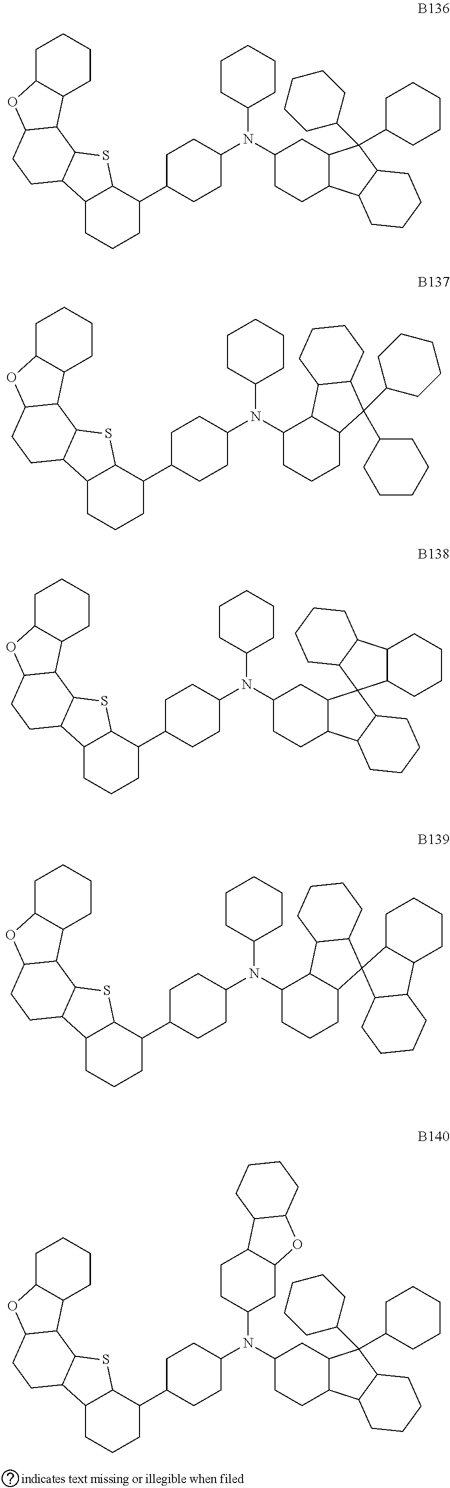 [SMILES: C1CCC(N(C2CCC(C3CCCC4C5CCC6OC7CCCCC7C6C5SC34)CC2)C2CCC3C4CCCCC4C(C4CCCCC4)(C4CCCCC4)C3C2)CC1 ; C1CCC(N(C2CCC(C3CCCC4C5CCC6OC7CCCCC7C6C5SC34)CC2)C2CCCC3C2C2CCCCC2C3(C2CCCCC2)C2CCCCC2)CC1 ; C1CCC(N(C2CCC(C3CCCC4C5CCC6OC7CCCCC7C6C5SC34)CC2)C2CCC3C4CCCCC4C4(C5CCCCC5C5CCCCC54)C3C2)CC1 ; C1CCC(N(C2CCC(C3CCCC4C5CCC6OC7CCCCC7C6C5SC34)CC2)C2CCCC3C2C2CCCCC2C32C3CCCCC3C3CCCCC32)CC1 ; C1CCC(C2(C3CCCCC3)C3CCCCC3C3CCC(N(C4CCC(C5CCCC6C7CCC8OC9CCCCC9C8C7SC56)CC4)C4CCC5C(C4)OC4CCCCC45)CC32)CC1]

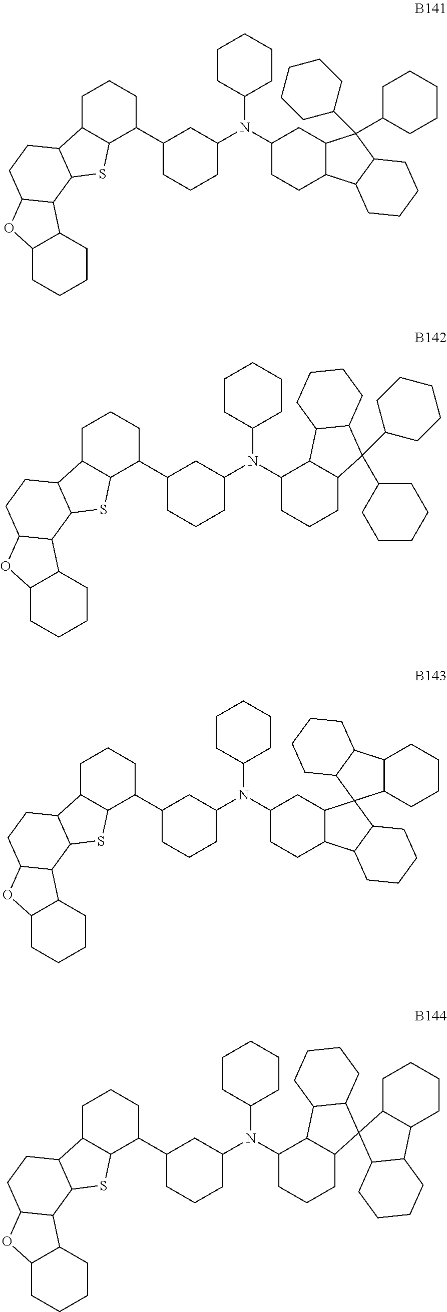 [SMILES: C1CCC(N(C2CCCC(C3CCCC4C5CCC6OC7CCCCC7C6C5SC34)C2)C2CCC3C4CCCCC4C(C4CCCCC4)(C4CCCCC4)C3C2)CC1 ; C1CCC(N(C2CCCC(C3CCCC4C5CCC6OC7CCCCC7C6C5SC34)C2)C2CCCC3C2C2CCCCC2C3(C2CCCCC2)C2CCCCC2)CC1 ; C1CCC(N(C2CCCC(C3CCCC4C5CCC6OC7CCCCC7C6C5SC34)C2)C2CCC3C4CCCCC4C4(C5CCCCC5C5CCCCC54)C3C2)CC1 ; C1CCC(N(C2CCCC(C3CCCC4C5CCC6OC7CCCCC7C6C5SC34)C2)C2CCCC3C2C2CCCCC2C32C3CCCCC3C3CCCCC32)CC1]

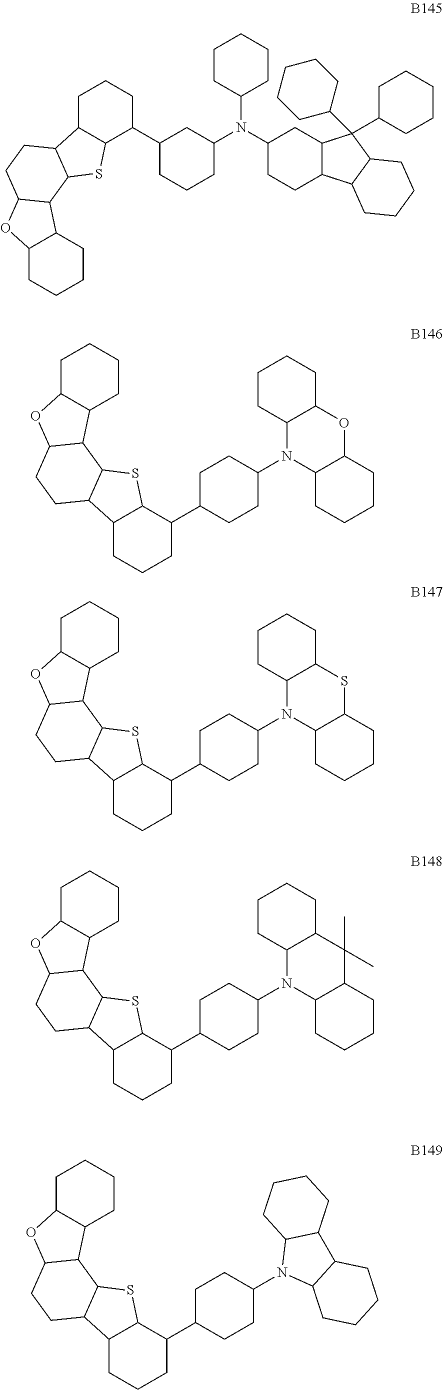 [SMILES: C1CCC(N(C2CCCC(C3CCCC4C5CCC6OC7CCCCC7C6C5SC34)C2)C2CCC3C4CCCCC4C(C4CCCCC4)(C4CCCCC4)C3C2)CC1 ; C1CCC2C(C1)OC1CCC3C4CCCC(C5CCC(N6C7CCCCC7OC7CCCCC76)CC5)C4SC3C12 ; C1CCC2C(C1)OC1CCC3C4CCCC(C5CCC(N6C7CCCCC7SC7CCCCC76)CC5)C4SC3C12 ; CC1(C)C2CCCCC2N(C2CCC(C3CCCC4C5CCC6OC7CCCCC7C6C5SC34)CC2)C2CCCCC21 ; C1CCC2C(C1)OC1CCC3C4CCCC(C5CCC(N6C7CCCCC7C7CCCCC76)CC5)C4SC3C12]

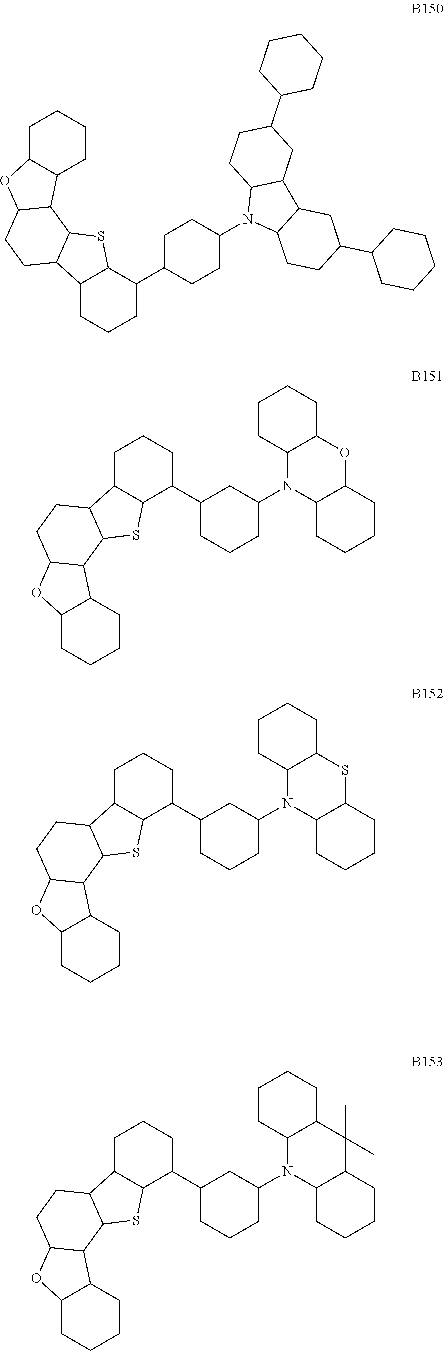 [SMILES: C1CCC(C2CCC3C(C2)C2CC(C4CCCCC4)CCC2N3C2CCC(C3CCCC4C5CCC6OC7CCCCC7C6C5SC34)CC2)CC1 ; C1CCC2C(C1)OC1CCC3C4CCCC(C5CCCC(N6C7CCCCC7OC7CCCCC76)C5)C4SC3C12 ; C1CCC2C(C1)OC1CCC3C4CCCC(C5CCCC(N6C7CCCCC7SC7CCCCC76)C5)C4SC3C12 ; CC1(C)C2CCCCC2N(C2CCCC(C3CCCC4C5CCC6OC7CCCCC7C6C5SC34)C2)C2CCCCC21]

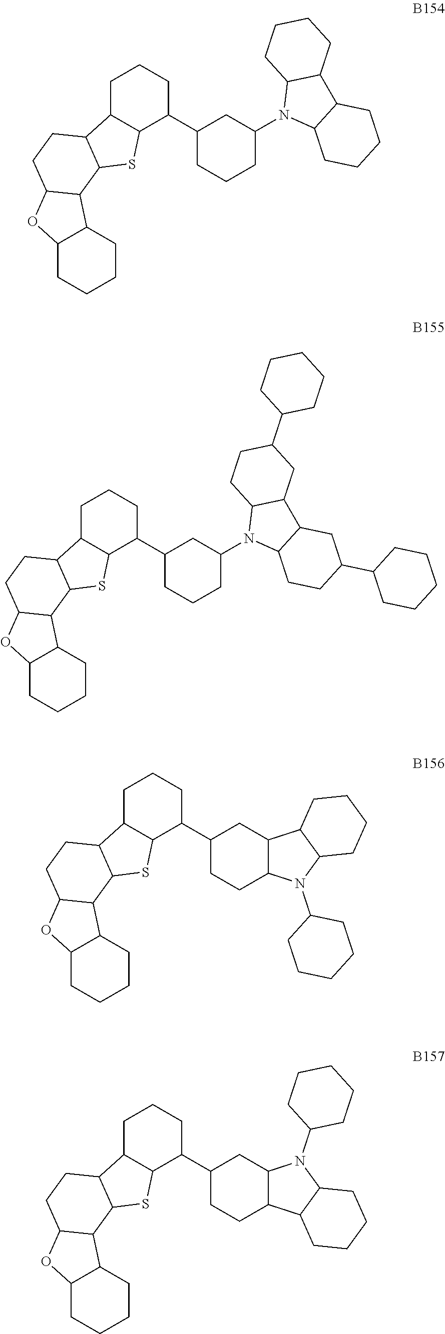 [SMILES: C1CCC2C(C1)OC1CCC3C4CCCC(C5CCCC(N6C7CCCCC7C7CCCCC76)C5)C4SC3C12 ; C1CCC(C2CCC3C(C2)C2CC(C4CCCCC4)CCC2N3C2CCCC(C3CCCC4C5CCC6OC7CCCCC7C6C5SC34)C2)CC1 ; C1CCC(N2C3CCCCC3C3CC(C4CCCC5C6CCC7OC8CCCCC8C7C6SC45)CCC32)CC1 ; C1CCC(N2C3CCCCC3C3CCC(C4CCCC5C6CCC7OC8CCCCC8C7C6SC45)CC32)CC1]

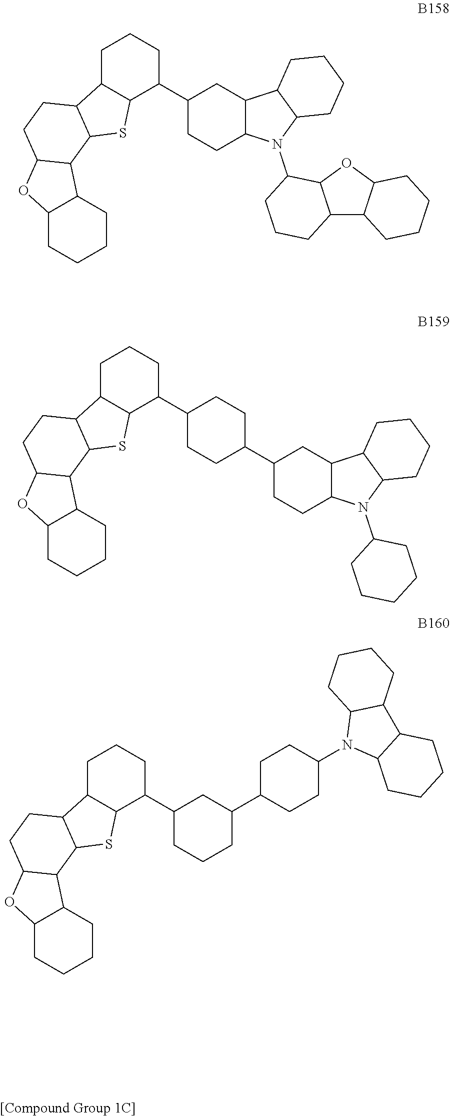 [SMILES: C1CCC2C(C1)OC1C2CCCC1N1C2CCCCC2C2CC(C3CCCC4C5CCC6OC7CCCCC7C6C5SC34)CCC21 ; C1CCC(N2C3CCCCC3C3CC(C4CCC(C5CCCC6C7CCC8OC9CCCCC9C8C7SC56)CC4)CCC32)CC1 ; C1CC(C2CCC(N3C4CCCCC4C4CCCCC43)CC2)CC(C2CCCC3C4CCC5OC6CCCCC6C5C4SC23)C1]

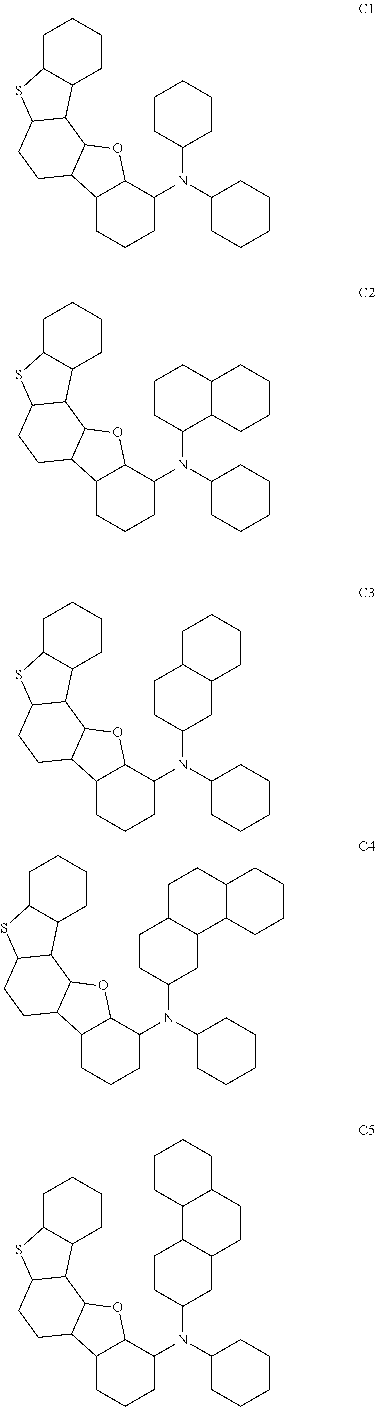

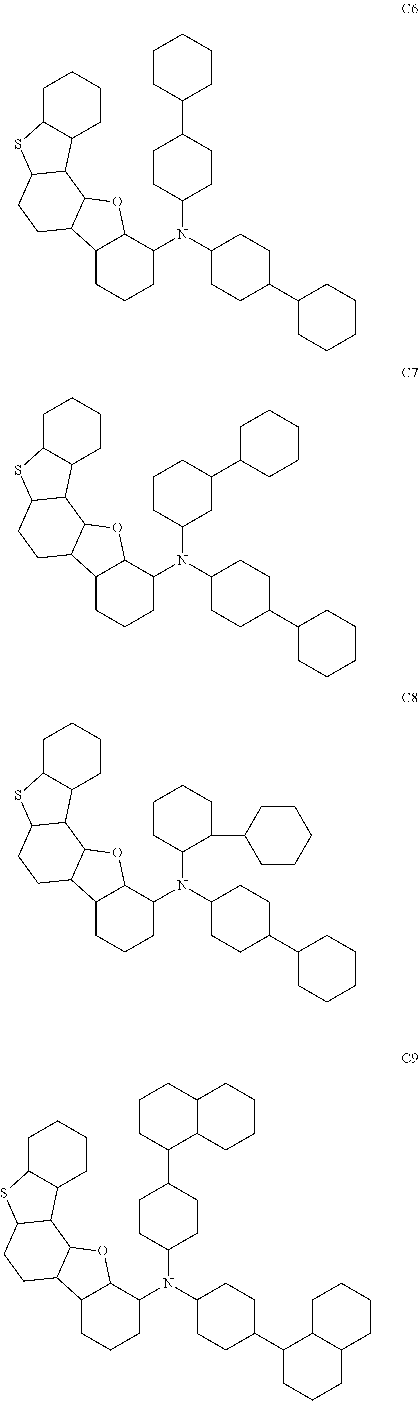 [SMILES: C1CCC(C2CCC(N(C3CCC(C4CCCCC4)CC3)C3CCCC4C5CCC6SC7CCCCC7C6C5OC43)CC2)CC1 ; C1CCC(C2CCC(N(C3CCCC(C4CCCCC4)C3)C3CCCC4C5CCC6SC7CCCCC7C6C5OC43)CC2)CC1 ; C1CCC(C2CCC(N(C3CCCCC3C3CCCCC3)C3CCCC4C5CCC6SC7CCCCC7C6C5OC43)CC2)CC1 ; C1CCC2C(C1)CCCC2C1CCC(N(C2CCC(C3CCCC4CCCCC43)CC2)C2CCCC3C4CCC5SC6CCCCC6C5C4OC32)CC1]

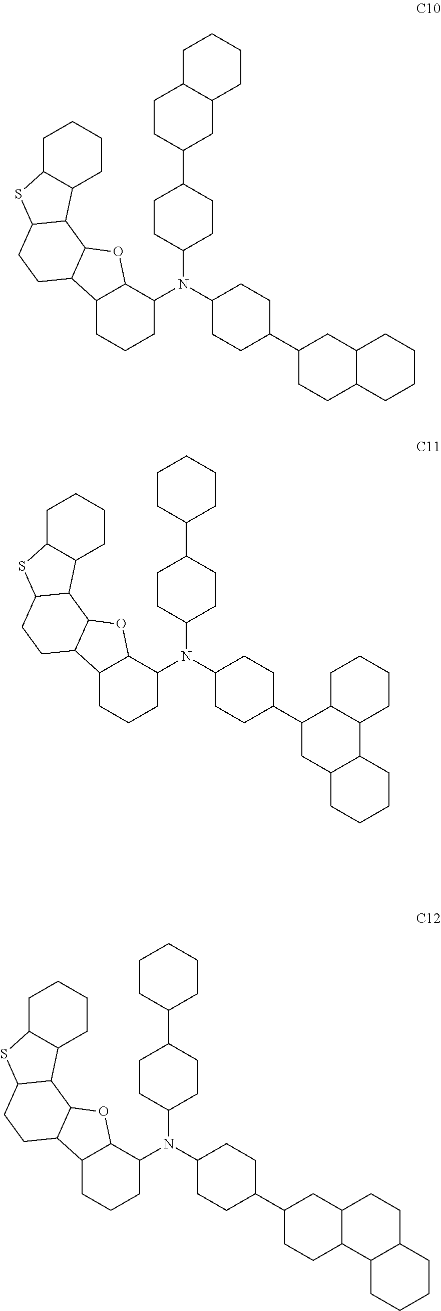 [SMILES: C1CCC2CC(C3CCC(N(C4CCC(C5CCC6CCCCC6C5)CC4)C4CCCC5C6CCC7SC8CCCCC8C7C6OC54)CC3)CCC2C1 ; C1CCC(C2CCC(N(C3CCC(C4CC5CCCCC5C5CCCCC45)CC3)C3CCCC4C5CCC6SC7CCCCC7C6C5OC43)CC2)CC1 ; C1CCC(C2CCC(N(C3CCC(C4CCC5C(CCC6CCCCC65)C4)CC3)C3CCCC4C5CCC6SC7CCCCC7C6C5OC43)CC2)CC1]

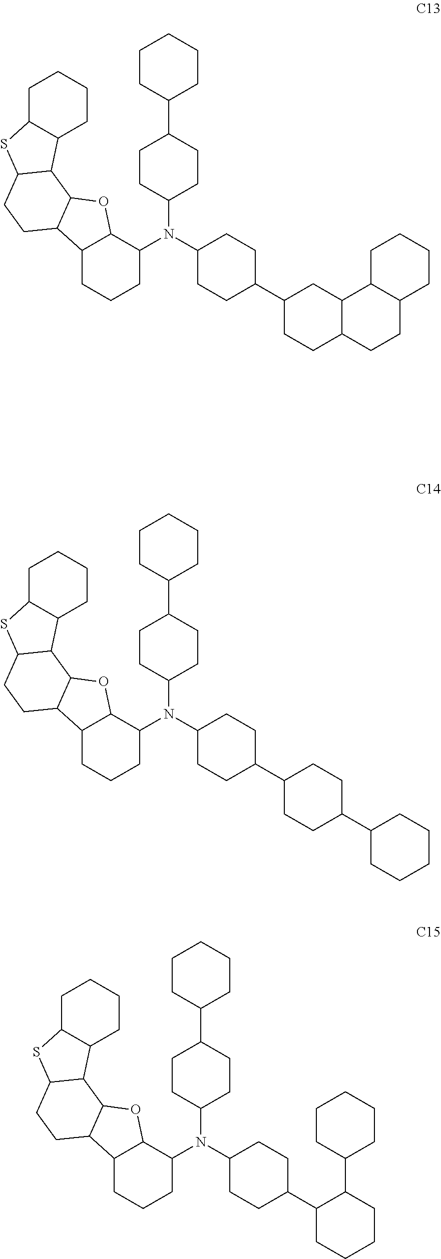 [SMILES: C1CCC(C2CCC(N(C3CCC(C4CCC5CCC6CCCCC6C5C4)CC3)C3CCCC4C5CCC6SC7CCCCC7C6C5OC43)CC2)CC1 ; C1CCC(C2CCC(C3CCC(N(C4CCC(C5CCCCC5)CC4)C4CCCC5C6CCC7SC8CCCCC8C7C6OC54)CC3)CC2)CC1 ; C1CCC(C2CCC(N(C3CCC(C4CCCCC4C4CCCCC4)CC3)C3CCCC4C5CCC6SC7CCCCC7C6C5OC43)CC2)CC1]

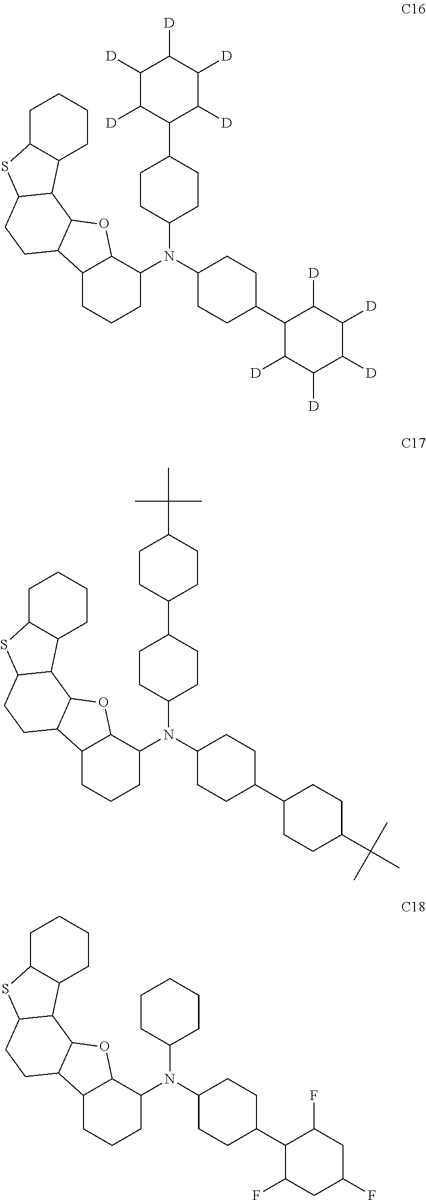 [SMILES: [2H]C1C([2H])C([2H])C(C2CCC(N(C3CCC(C4C([2H])C([2H])C([2H])C([2H])C4[2H])CC3)C3CCCC4C5CCC6SC7CCCCC7C6C5OC43)CC2)C([2H])C1[2H] ; CC(C)(C)C1CCC(C2CCC(N(C3CCC(C4CCC(C(C)(C)C)CC4)CC3)C3CCCC4C5CCC6SC7CCCCC7C6C5OC43)CC2)CC1 ; FC1CC(F)C(C2CCC(N(C3CCCCC3)C3CCCC4C5CCC6SC7CCCCC7C6C5OC43)CC2)C(F)C1]

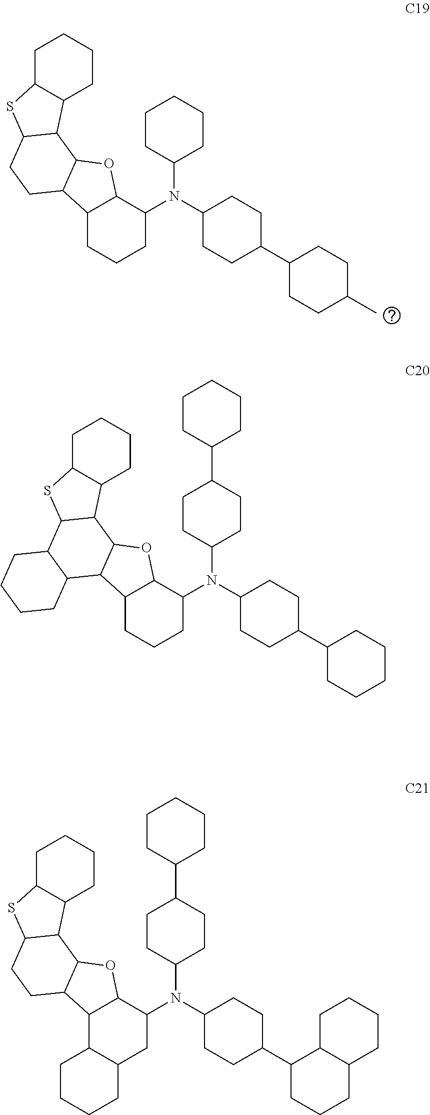 [SMILES: CC1CCC(C2CCC(N(C3CCCCC3)C3CCCC4C5CCC6SC7CCCCC7C6C5OC43)CC2)CC1 ; C1CCC(C2CCC(N(C3CCC(C4CCCCC4)CC3)C3CCCC4C3OC3C4C4CCCCC4C4SC5CCCCC5C34)CC2)CC1 ; C1CCC(C2CCC(N(C3CCC(C4CCCC5CCCCC54)CC3)C3CC4CCCCC4C4C5CCC6SC7CCCCC7C6C5OC43)CC2)CC1]

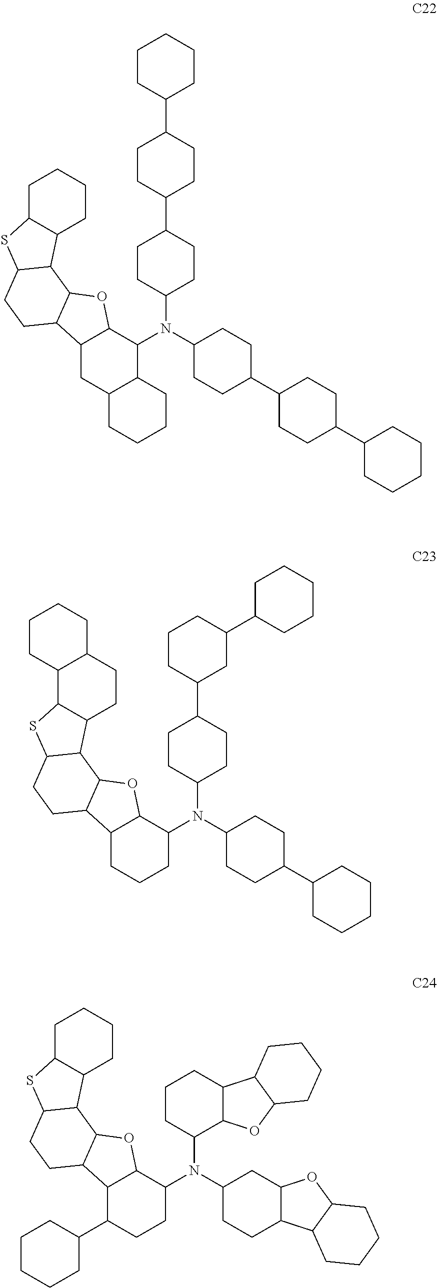 [SMILES: C1CCC(C2CCC(C3CCC(N(C4CCC(C5CCC(C6CCCCC6)CC5)CC4)C4C5CCCCC5CC5C6CCC7SC8CCCCC8C7C6OC54)CC3)CC2)CC1 ; C1CCC(C2CCC(N(C3CCC(C4CCCC(C5CCCCC5)C4)CC3)C3CCCC4C5CCC6SC7C8CCCCC8CCC7C6C5OC43)CC2)CC1 ; C1CCC(C2CCC(N(C3CCC4C(C3)OC3CCCCC34)C3CCCC4C5CCCCC5OC43)C3OC4C(CCC5SC6CCCCC6C54)C23)CC1]

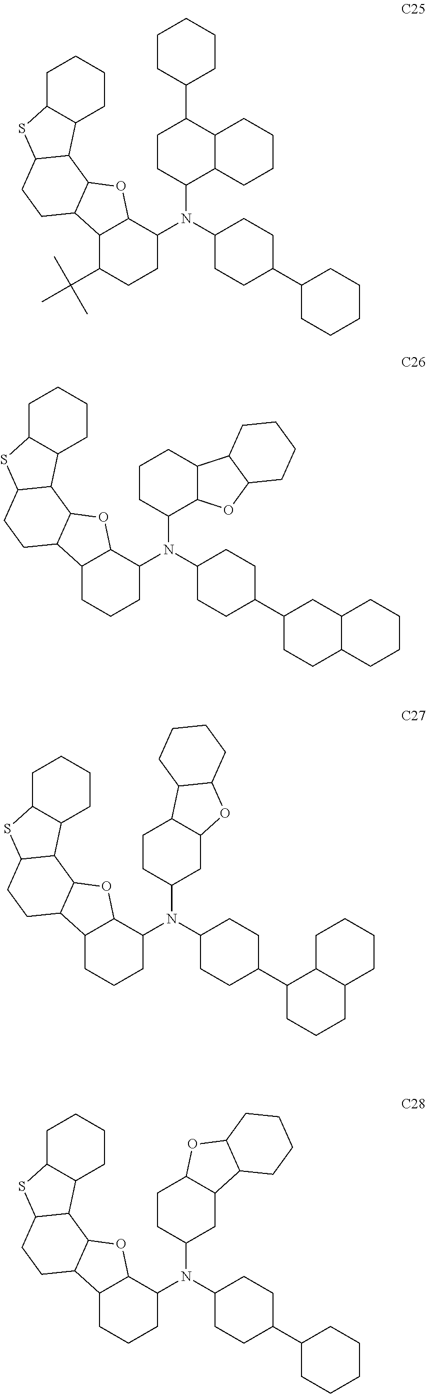 [SMILES: CC(C)(C)C1CCC(N(C2CCC(C3CCCCC3)CC2)C2CCC(C3CCCCC3)C3CCCCC32)C2OC3C(CCC4SC5CCCCC5C43)C21 ; C1CCC2CC(C3CCC(N(C4CCCC5C6CCCCC6OC54)C4CCCC5C6CCC7SC8CCCCC8C7C6OC54)CC3)CCC2C1 ; C1CCC2C(C1)CCCC2C1CCC(N(C2CCC3C(C2)OC2CCCCC23)C2CCCC3C4CCC5SC6CCCCC6C5C4OC32)CC1 ; C1CCC(C2CCC(N(C3CCC4OC5CCCCC5C4C3)C3CCCC4C5CCC6SC7CCCCC7C6C5OC43)CC2)CC1]

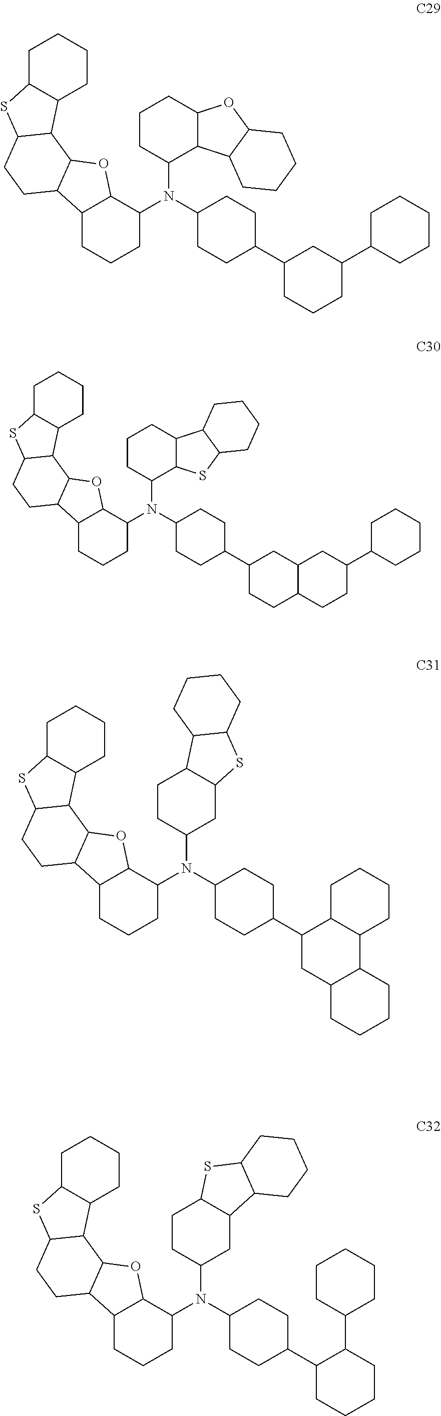 [SMILES: C1CCC(C2CCCC(C3CCC(N(C4CCCC5C6CCC7SC8CCCCC8C7C6OC54)C4CCCC5OC6CCCCC6C54)CC3)C2)CC1 ; C1CCC(C2CCC3CCC(C4CCC(N(C5CCCC6C7CCC8SC9CCCCC9C8C7OC65)C5CCCC6C7CCCCC7SC65)CC4)CC3C2)CC1 ; C1CCC2C(C1)CC(C1CCC(N(C3CCC4C(C3)SC3CCCCC34)C3CCCC4C5CCC6SC7CCCCC7C6C5OC43)CC1)C1CCCCC21 ; C1CCC(C2CCCCC2C2CCC(N(C3CCC4SC5CCCCC5C4C3)C3CCCC4C5CCC6SC7CCCCC7C6C5OC43)CC2)CC1]

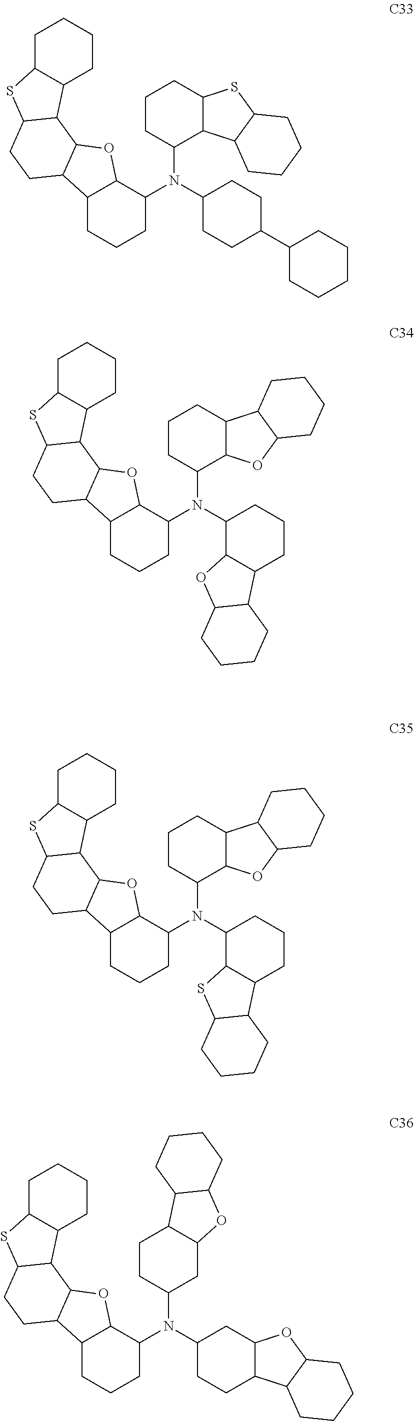 [SMILES: C1CCC(C2CCC(N(C3CCCC4C5CCC6SC7CCCCC7C6C5OC43)C3CCCC4SC5CCCCC5C43)CC2)CC1 ; C1CCC2C(C1)OC1C2CCCC1N(C1CCCC2C3CCCCC3OC21)C1CCCC2C3CCC4SC5CCCCC5C4C3OC21 ; C1CCC2C(C1)OC1C2CCCC1N(C1CCCC2C3CCC4SC5CCCCC5C4C3OC21)C1CCCC2C3CCCCC3SC21 ; C1CCC2C(C1)OC1CC(N(C3CCC4C(C3)OC3CCCCC34)C3CCCC4C5CCC6SC7CCCCC7C6C5OC43)CCC12]

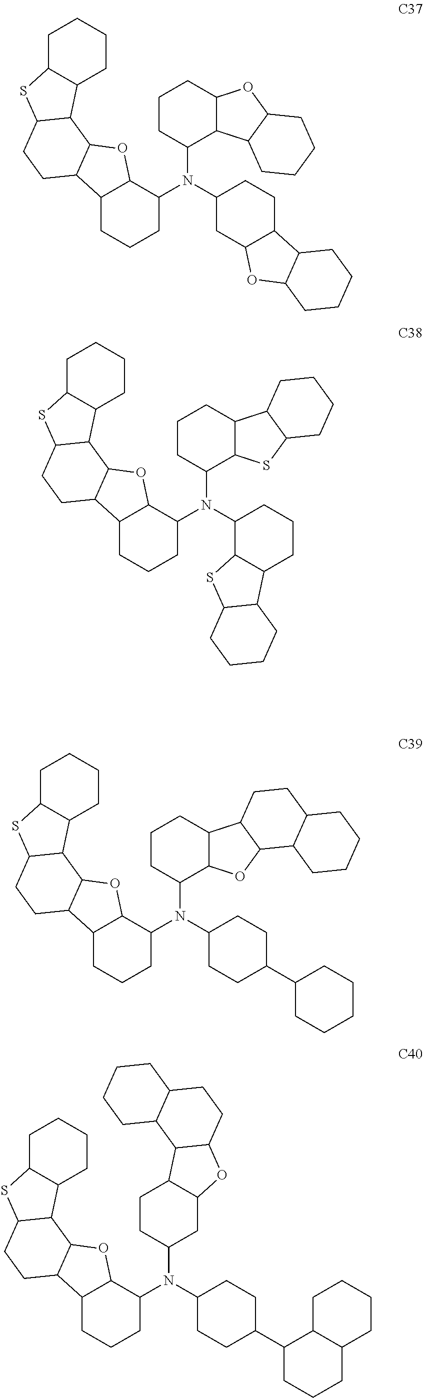 [SMILES: C1CCC2C(C1)OC1CC(N(C3CCCC4C5CCC6SC7CCCCC7C6C5OC43)C3CCCC4OC5CCCCC5C43)CCC12 ; C1CCC2C(C1)SC1C2CCCC1N(C1CCCC2C3CCC4SC5CCCCC5C4C3OC21)C1CCCC2C3CCCCC3SC21 ; C1CCC(C2CCC(N(C3CCCC4C5CCC6CCCCC6C5OC43)C3CCCC4C5CCC6SC7CCCCC7C6C5OC43)CC2)CC1 ; C1CCC2C(C1)CCCC2C1CCC(N(C2CCC3C(C2)OC2CCC4CCCCC4C23)C2CCCC3C4CCC5SC6CCCCC6C5C4OC32)CC1]

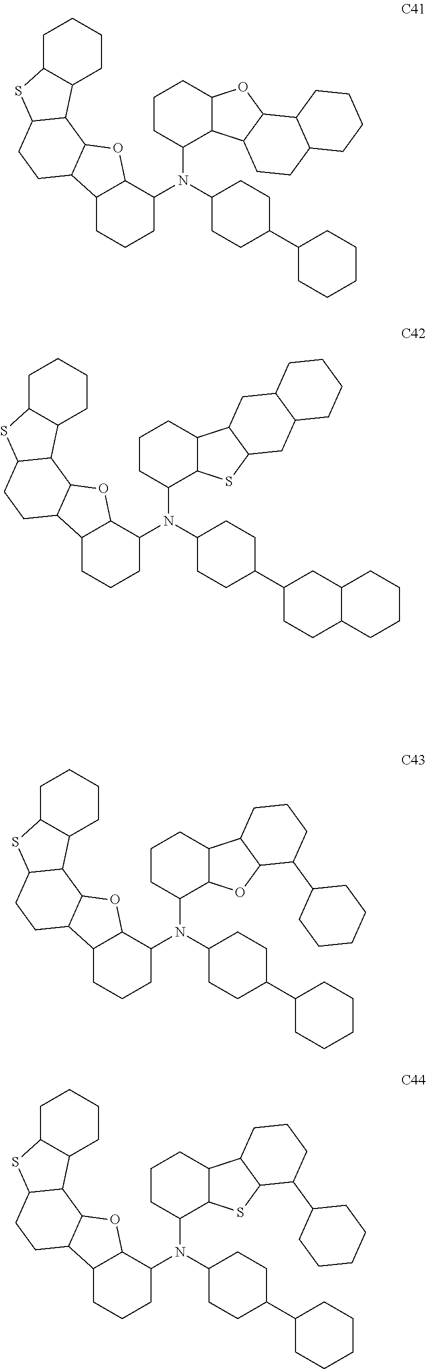 [SMILES: C1CCC(C2CCC(N(C3CCCC4C5CCC6SC7CCCCC7C6C5OC43)C3CCCC4OC5C6CCCCC6CCC5C43)CC2)CC1 ; C1CCC2CC(C3CCC(N(C4CCCC5C6CCC7SC8CCCCC8C7C6OC54)C4CCCC5C6CC7CCCCC7CC6SC54)CC3)CCC2C1 ; C1CCC(C2CCC(N(C3CCCC4C5CCCC(C6CCCCC6)C5OC43)C3CCCC4C5CCC6SC7CCCCC7C6C5OC43)CC2)CC1 ; C1CCC(C2CCC(N(C3CCCC4C5CCC6SC7CCCCC7C6C5OC43)C3CCCC4C5CCCC(C6CCCCC6)C5SC43)CC2)CC1]

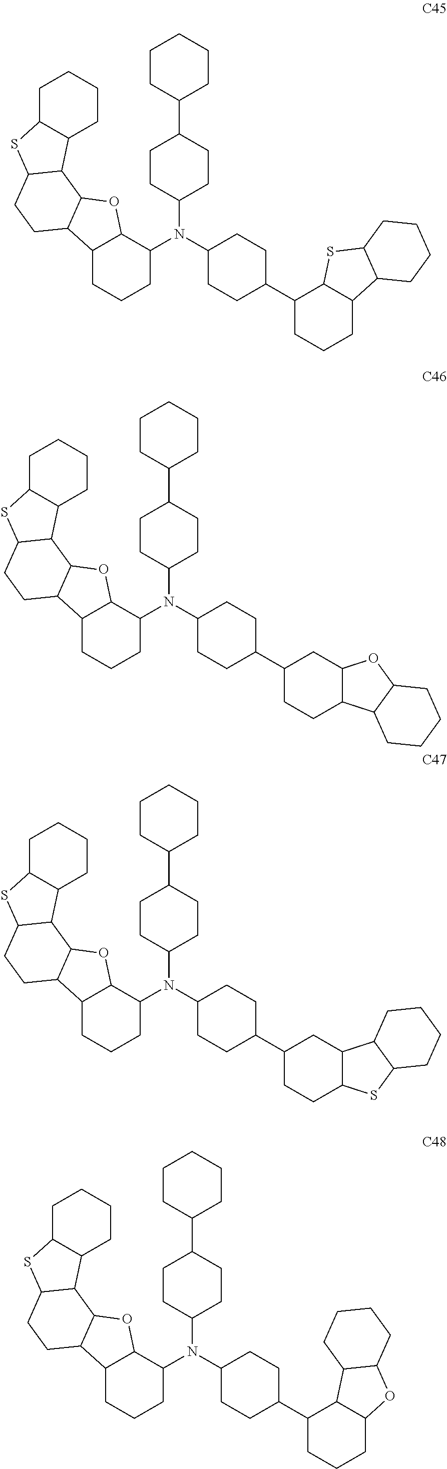 [SMILES: C1CCC(C2CCC(N(C3CCC(C4CCCC5C6CCCCC6SC45)CC3)C3CCCC4C5CCC6SC7CCCCC7C6C5OC43)CC2)CC1 ; C1CCC(C2CCC(N(C3CCC(C4CCC5C(C4)OC4CCCCC45)CC3)C3CCCC4C5CCC6SC7CCCCC7C6C5OC43)CC2)CC1 ; C1CCC(C2CCC(N(C3CCC(C4CCC5SC6CCCCC6C5C4)CC3)C3CCCC4C5CCC6SC7CCCCC7C6C5OC43)CC2)CC1 ; C1CCC(C2CCC(N(C3CCC(C4CCCC5OC6CCCCC6C54)CC3)C3CCCC4C5CCC6SC7CCCCC7C6C5OC43)CC2)CC1]

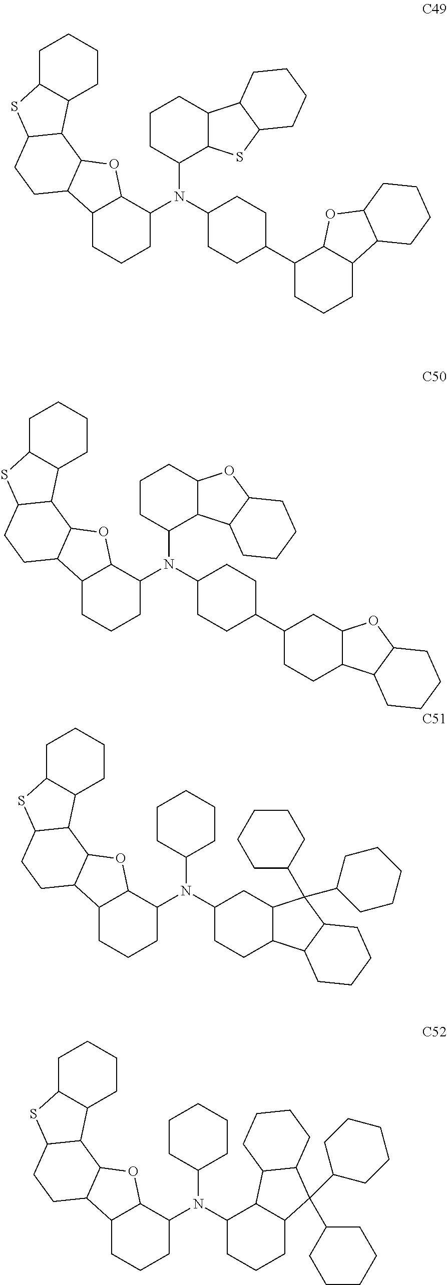 [SMILES: C1CCC2C(C1)OC1C(C3CCC(N(C4CCCC5C6CCC7SC8CCCCC8C7C6OC54)C4CCCC5C6CCCCC6SC54)CC3)CCCC21 ; C1CCC2C(C1)OC1CC(C3CCC(N(C4CCCC5C6CCC7SC8CCCCC8C7C6OC54)C4CCCC5OC6CCCCC6C54)CC3)CCC12 ; C1CCC(N(C2CCC3C4CCCCC4C(C4CCCCC4)(C4CCCCC4)C3C2)C2CCCC3C4CCC5SC6CCCCC6C5C4OC32)CC1 ; C1CCC(N(C2CCCC3C4CCC5SC6CCCCC6C5C4OC32)C2CCCC3C2C2CCCCC2C3(C2CCCCC2)C2CCCCC2)CC1]

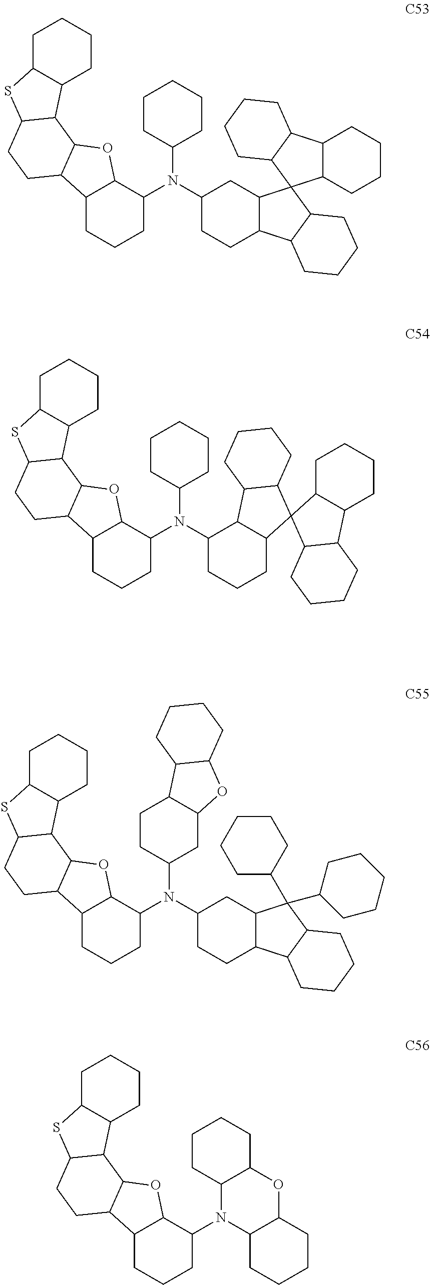

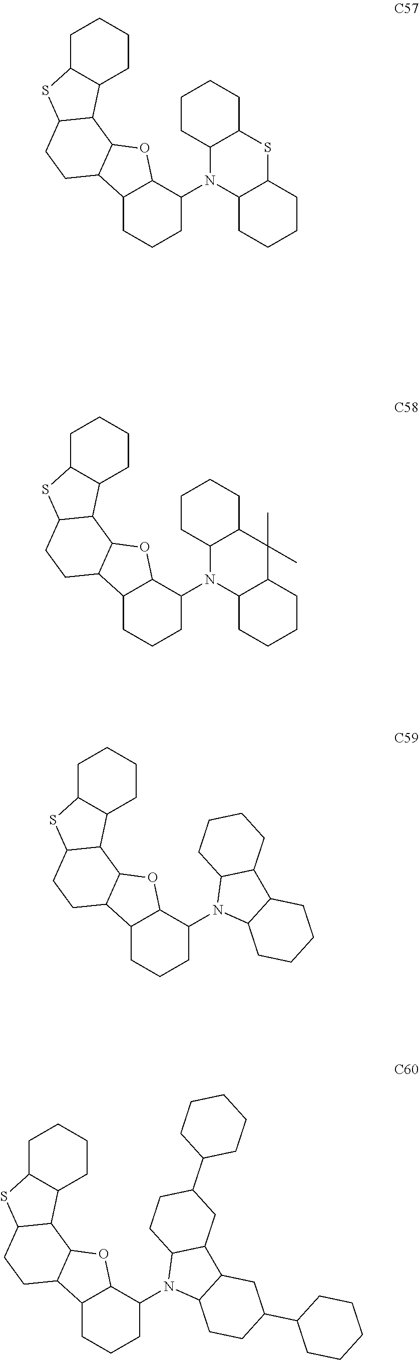

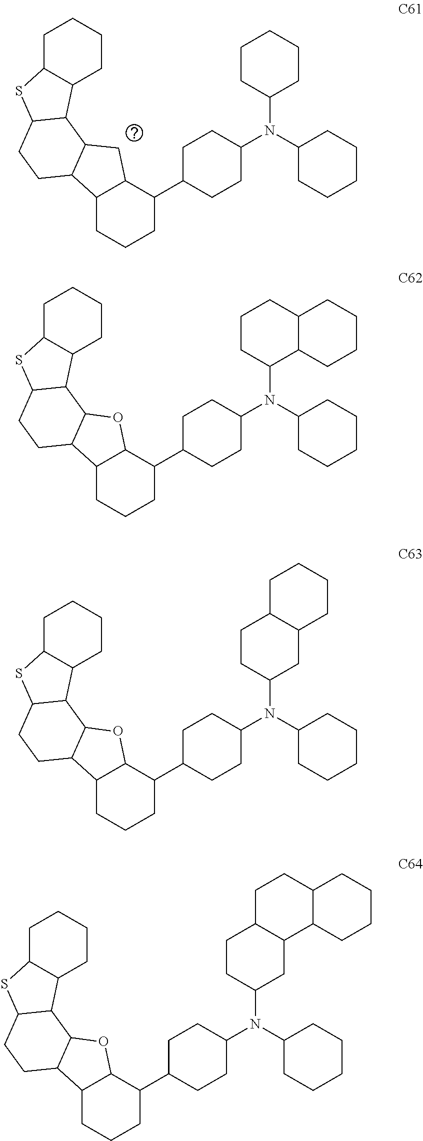 [SMILES: C1CCC(N(C2CCCCC2)C2CCC(C3CCCC4C3CC3C4CCC4SC5CCCCC5C43)CC2)CC1 ; C1CCC(N(C2CCC(C3CCCC4C5CCC6SC7CCCCC7C6C5OC34)CC2)C2CCCC3CCCCC32)CC1 ; C1CCC(N(C2CCC(C3CCCC4C5CCC6SC7CCCCC7C6C5OC34)CC2)C2CCC3CCCCC3C2)CC1 ; C1CCC(N(C2CCC(C3CCCC4C5CCC6SC7CCCCC7C6C5OC34)CC2)C2CCC3CCC4CCCCC4C3C2)CC1]

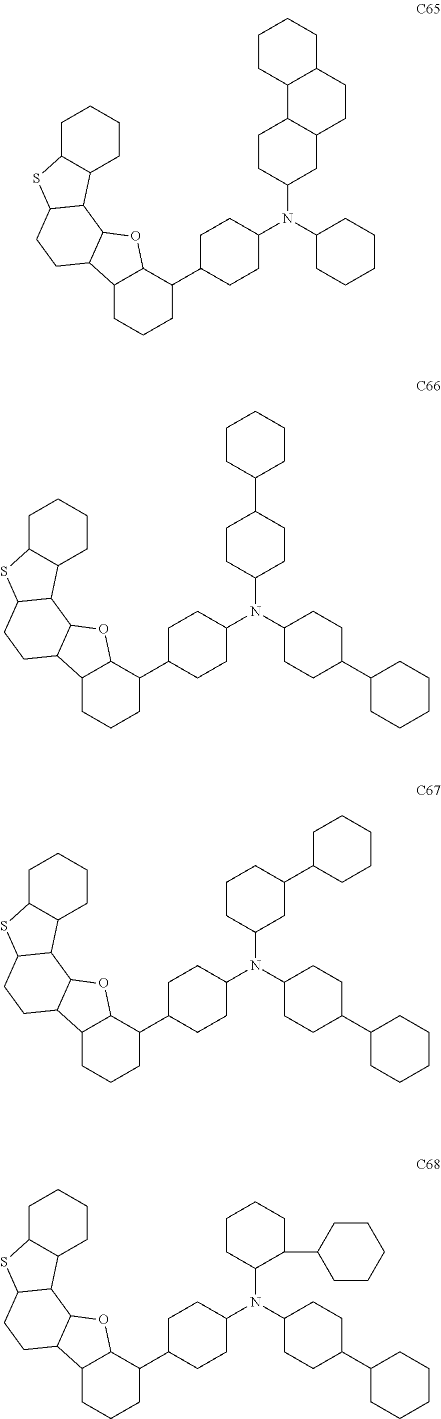 [SMILES: C1CCC(N(C2CCC(C3CCCC4C5CCC6SC7CCCCC7C6C5OC34)CC2)C2CCC3C(CCC4CCCCC43)C2)CC1 ; C1CCC(C2CCC(N(C3CCC(C4CCCCC4)CC3)C3CCC(C4CCCC5C6CCC7SC8CCCCC8C7C6OC45)CC3)CC2)CC1 ; C1CCC(C2CCC(N(C3CCC(C4CCCC5C6CCC7SC8CCCCC8C7C6OC45)CC3)C3CCCC(C4CCCCC4)C3)CC2)CC1 ; C1CCC(C2CCC(N(C3CCC(C4CCCC5C6CCC7SC8CCCCC8C7C6OC45)CC3)C3CCCCC3C3CCCCC3)CC2)CC1]

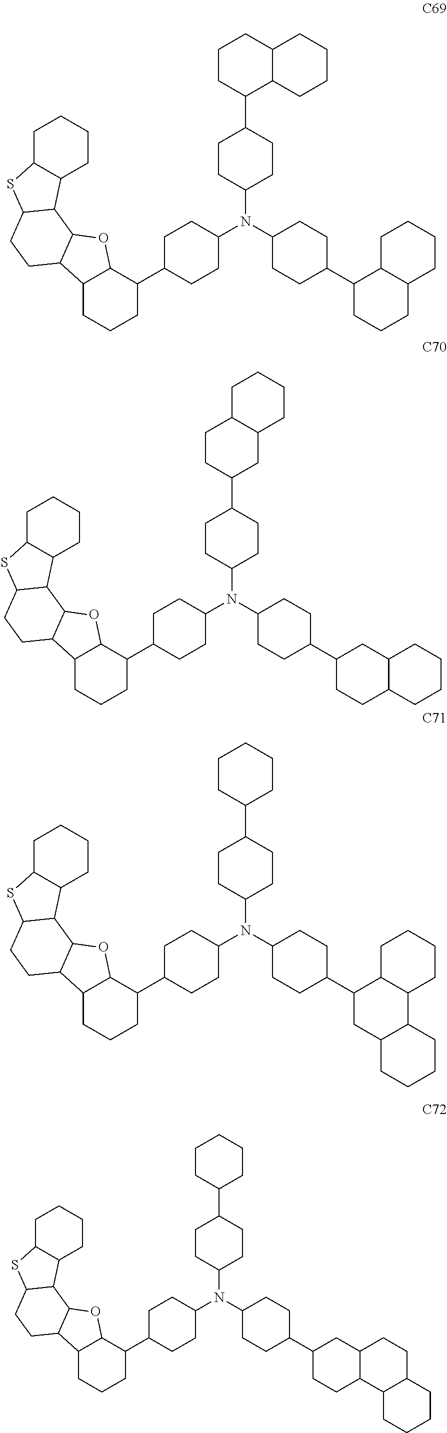 [SMILES: C1CCC2C(C1)CCCC2C1CCC(N(C2CCC(C3CCCC4CCCCC43)CC2)C2CCC(C3CCCC4C5CCC6SC7CCCCC7C6C5OC34)CC2)CC1 ; C1CCC2CC(C3CCC(N(C4CCC(C5CCC6CCCCC6C5)CC4)C4CCC(C5CCCC6C7CCC8SC9CCCCC9C8C7OC56)CC4)CC3)CCC2C1 ; C1CCC(C2CCC(N(C3CCC(C4CC5CCCCC5C5CCCCC45)CC3)C3CCC(C4CCCC5C6CCC7SC8CCCCC8C7C6OC45)CC3)CC2)CC1 ; C1CCC(C2CCC(N(C3CCC(C4CCC5C(CCC6CCCCC65)C4)CC3)C3CCC(C4CCCC5C6CCC7SC8CCCCC8C7C6OC45)CC3)CC2)CC1]

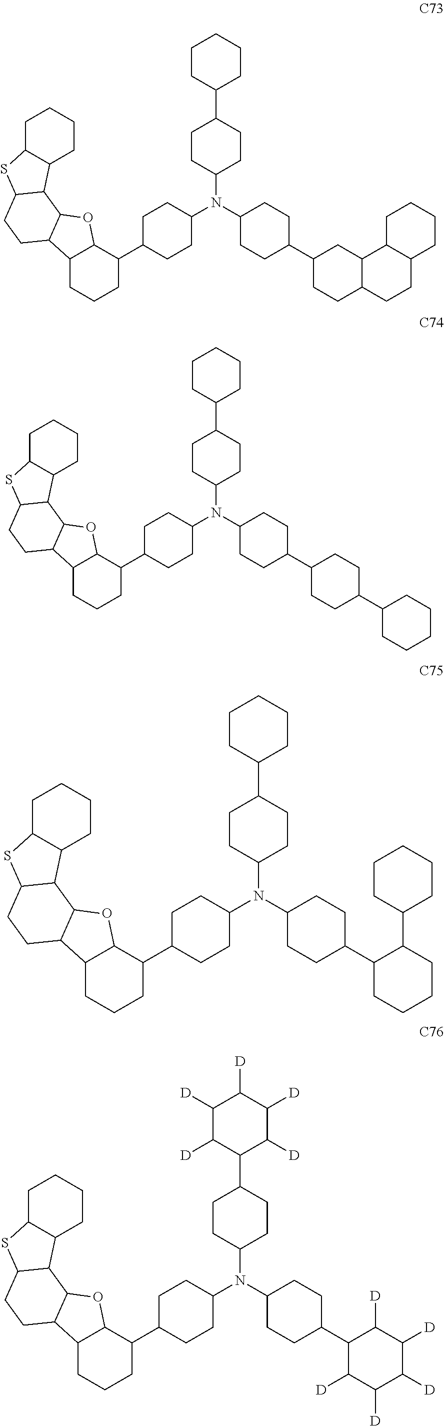 [SMILES: C1CCC(C2CCC(N(C3CCC(C4CCC5CCC6CCCCC6C5C4)CC3)C3CCC(C4CCCC5C6CCC7SC8CCCCC8C7C6OC45)CC3)CC2)CC1 ; C1CCC(C2CCC(C3CCC(N(C4CCC(C5CCCCC5)CC4)C4CCC(C5CCCC6C7CCC8SC9CCCCC9C8C7OC56)CC4)CC3)CC2)CC1 ; C1CCC(C2CCC(N(C3CCC(C4CCCCC4C4CCCCC4)CC3)C3CCC(C4CCCC5C6CCC7SC8CCCCC8C7C6OC45)CC3)CC2)CC1 ; [2H]C1C([2H])C([2H])C(C2CCC(N(C3CCC(C4CCCC5C6CCC7SC8CCCCC8C7C6OC45)CC3)C3CCC(C4C([2H])C([2H])C([2H])C([2H])C4[2H])CC3)CC2)C([2H])C1[2H]]

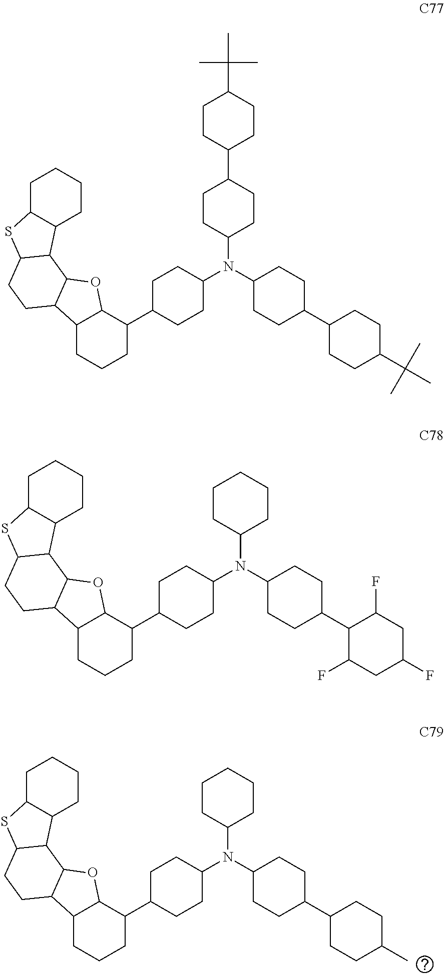 [SMILES: CC(C)(C)C1CCC(C2CCC(N(C3CCC(C4CCC(C(C)(C)C)CC4)CC3)C3CCC(C4CCCC5C6CCC7SC8CCCCC8C7C6OC45)CC3)CC2)CC1 ; FC1CC(F)C(C2CCC(N(C3CCCCC3)C3CCC(C4CCCC5C6CCC7SC8CCCCC8C7C6OC45)CC3)CC2)C(F)C1 ; CC1CCC(C2CCC(N(C3CCCCC3)C3CCC(C4CCCC5C6CCC7SC8CCCCC8C7C6OC45)CC3)CC2)CC1]

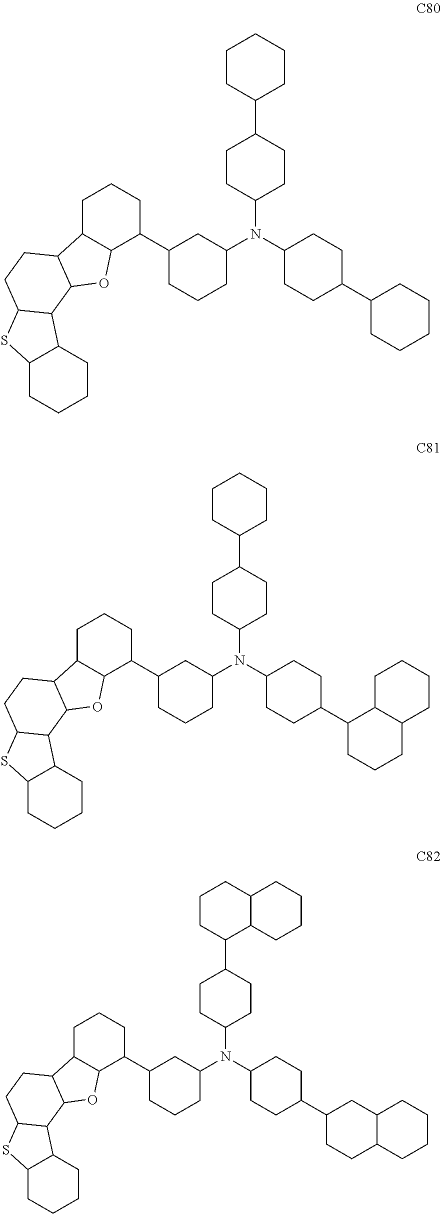 [SMILES: C1CCC(C2CCC(N(C3CCC(C4CCCCC4)CC3)C3CCCC(C4CCCC5C6CCC7SC8CCCCC8C7C6OC45)C3)CC2)CC1 ; C1CCC(C2CCC(N(C3CCC(C4CCCC5CCCCC54)CC3)C3CCCC(C4CCCC5C6CCC7SC8CCCCC8C7C6OC45)C3)CC2)CC1 ; C1CCC2CC(C3CCC(N(C4CCC(C5CCCC6CCCCC65)CC4)C4CCCC(C5CCCC6C7CCC8SC9CCCCC9C8C7OC56)C4)CC3)CCC2C1]

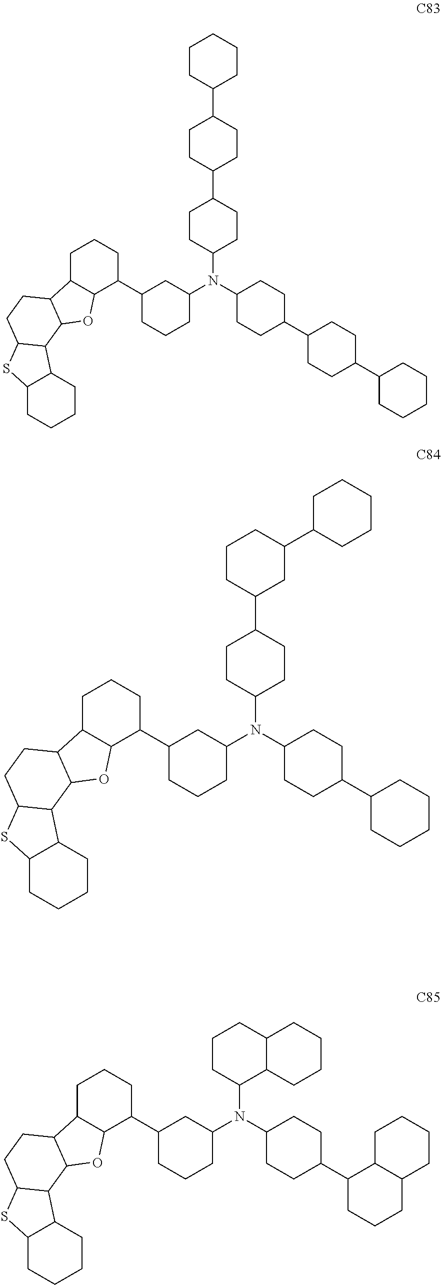 [SMILES: C1CCC(C2CCC(C3CCC(N(C4CCC(C5CCC(C6CCCCC6)CC5)CC4)C4CCCC(C5CCCC6C7CCC8SC9CCCCC9C8C7OC56)C4)CC3)CC2)CC1 ; C1CCC(C2CCC(N(C3CCC(C4CCCC(C5CCCCC5)C4)CC3)C3CCCC(C4CCCC5C6CCC7SC8CCCCC8C7C6OC45)C3)CC2)CC1 ; C1CCC2C(C1)CCCC2C1CCC(N(C2CCCC(C3CCCC4C5CCC6SC7CCCCC7C6C5OC34)C2)C2CCCC3CCCCC32)CC1]

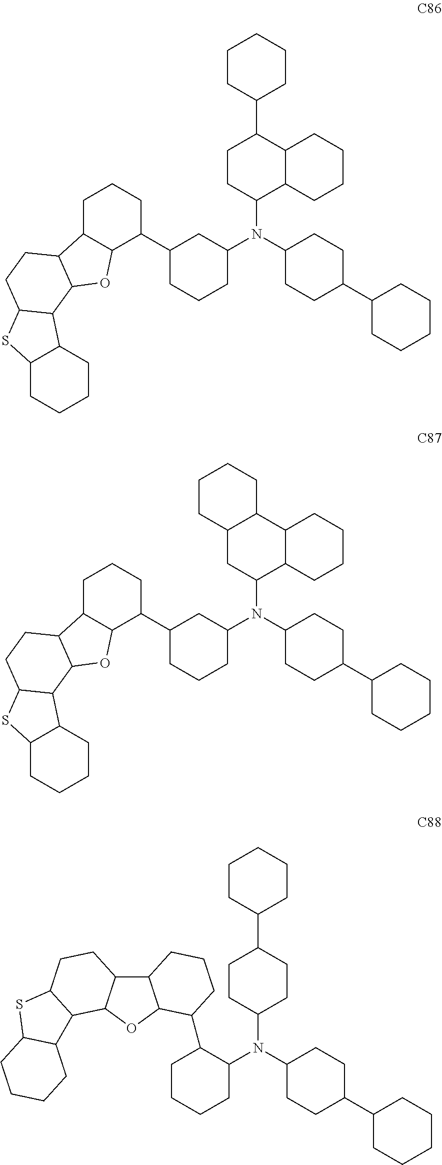 [SMILES: C1CCC(C2CCC(N(C3CCCC(C4CCCC5C6CCC7SC8CCCCC8C7C6OC45)C3)C3CCC(C4CCCCC4)C4CCCCC43)CC2)CC1 ; C1CCC(C2CCC(N(C3CCCC(C4CCCC5C6CCC7SC8CCCCC8C7C6OC45)C3)C3CC4CCCCC4C4CCCCC43)CC2)CC1 ; C1CCC(C2CCC(N(C3CCC(C4CCCCC4)CC3)C3CCCCC3C3CCCC4C5CCC6SC7CCCCC7C6C5OC43)CC2)CC1]

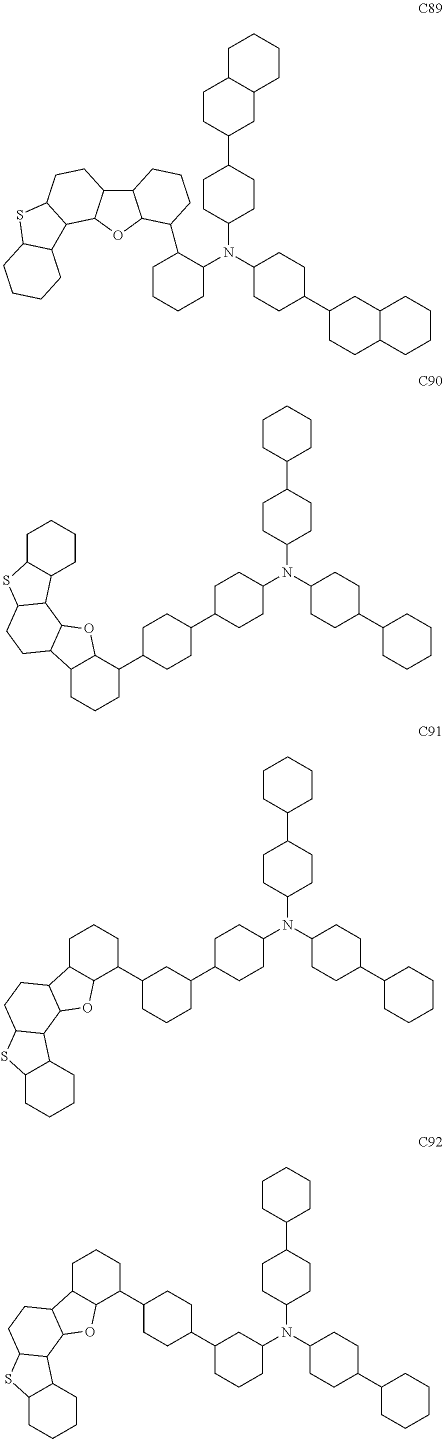 [SMILES: C1CCC2CC(C3CCC(N(C4CCC(C5CCC6CCCCC6C5)CC4)C4CCCCC4C4CCCC5C6CCC7SC8CCCCC8C7C6OC54)CC3)CCC2C1 ; C1CCC(C2CCC(N(C3CCC(C4CCCCC4)CC3)C3CCC(C4CCC(C5CCCC6C7CCC8SC9CCCCC9C8C7OC56)CC4)CC3)CC2)CC1 ; C1CCC(C2CCC(N(C3CCC(C4CCCCC4)CC3)C3CCC(C4CCCC(C5CCCC6C7CCC8SC9CCCCC9C8C7OC56)C4)CC3)CC2)CC1 ; C1CCC(C2CCC(N(C3CCC(C4CCCCC4)CC3)C3CCCC(C4CCC(C5CCCC6C7CCC8SC9CCCCC9C8C7OC56)CC4)C3)CC2)CC1]

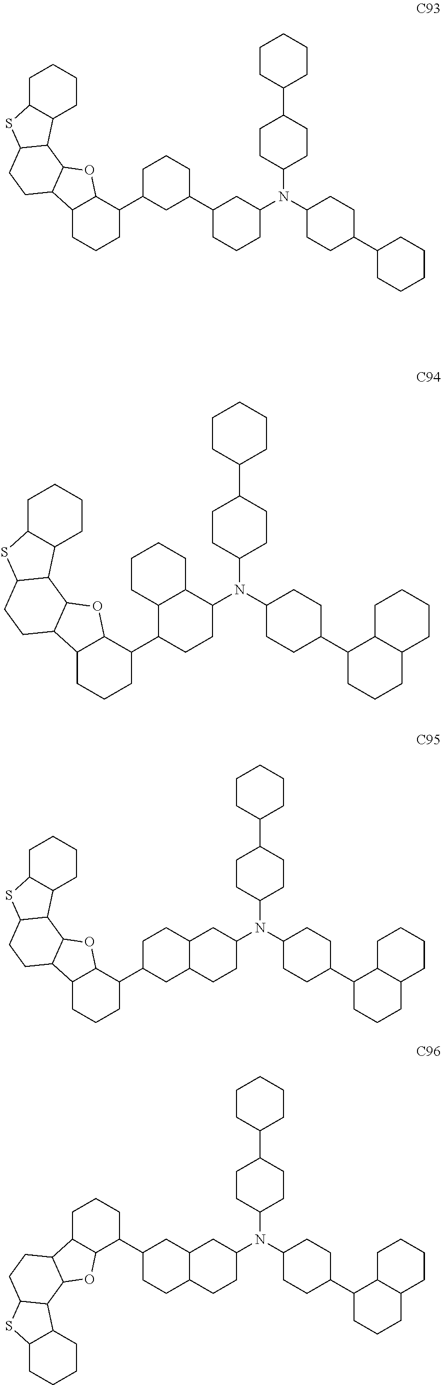 [SMILES: C1CCC(C2CCC(N(C3CCC(C4CCCCC4)CC3)C3CCCC(C4CCCC(C5CCCC6C7CCC8SC9CCCCC9C8C7OC56)C4)C3)CC2)CC1 ; C1CCC(C2CCC(N(C3CCC(C4CCCC5CCCCC54)CC3)C3CCC(C4CCCC5C6CCC7SC8CCCCC8C7C6OC45)C4CCCCC43)CC2)CC1 ; C1CCC(C2CCC(N(C3CCC(C4CCCC5CCCCC54)CC3)C3CCC4CC(C5CCCC6C7CCC8SC9CCCCC9C8C7OC56)CCC4C3)CC2)CC1 ; C1CCC(C2CCC(N(C3CCC(C4CCCC5CCCCC54)CC3)C3CCC4CCC(C5CCCC6C7CCC8SC9CCCCC9C8C7OC56)CC4C3)CC2)CC1]

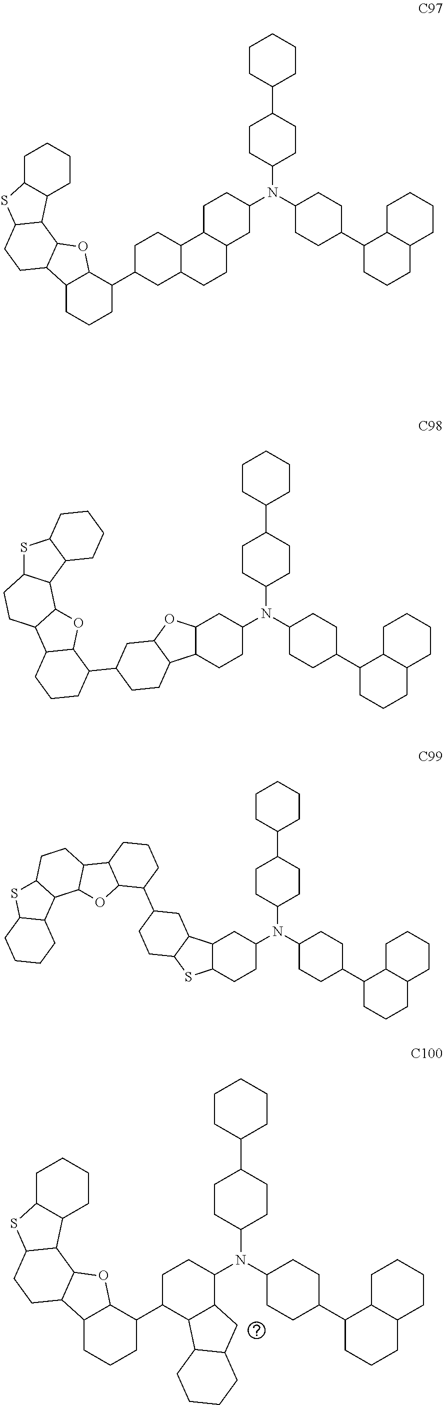 [SMILES: C1CCC(C2CCC(N(C3CCC(C4CCCC5CCCCC54)CC3)C3CCC4C(CCC5CC(C6CCCC7C8CCC9SC%10CCCCC%10C9C8OC67)CCC54)C3)CC2)CC1 ; C1CCC(C2CCC(N(C3CCC(C4CCCC5CCCCC54)CC3)C3CCC4C(C3)OC3CC(C5CCCC6C7CCC8SC9CCCCC9C8C7OC56)CCC34)CC2)CC1 ; C1CCC(C2CCC(N(C3CCC(C4CCCC5CCCCC54)CC3)C3CCC4SC5CCC(C6CCCC7C8CCC9SC%10CCCCC%10C9C8OC67)CC5C4C3)CC2)CC1 ; C1CCC(C2CCC(N(C3CCC(C4CCCC5CCCCC54)CC3)C3CCC(C4CCCC5C6CCC7SC8CCCCC8C7C6OC54)C4C5CCCCC5CC43)CC2)CC1]

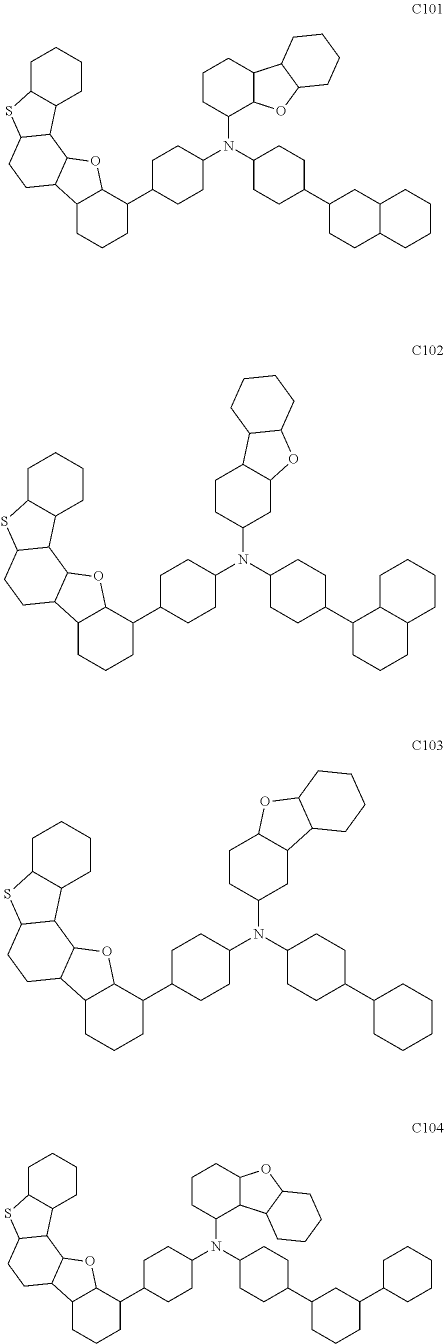 [SMILES: C1CCC2CC(C3CCC(N(C4CCC(C5CCCC6C7CCC8SC9CCCCC9C8C7OC56)CC4)C4CCCC5C6CCCCC6OC54)CC3)CCC2C1 ; C1CCC2C(C1)CCCC2C1CCC(N(C2CCC(C3CCCC4C5CCC6SC7CCCCC7C6C5OC34)CC2)C2CCC3C(C2)OC2CCCCC23)CC1 ; C1CCC(C2CCC(N(C3CCC(C4CCCC5C6CCC7SC8CCCCC8C7C6OC45)CC3)C3CCC4OC5CCCCC5C4C3)CC2)CC1 ; C1CCC(C2CCCC(C3CCC(N(C4CCC(C5CCCC6C7CCC8SC9CCCCC9C8C7OC56)CC4)C4CCCC5OC6CCCCC6C54)CC3)C2)CC1]

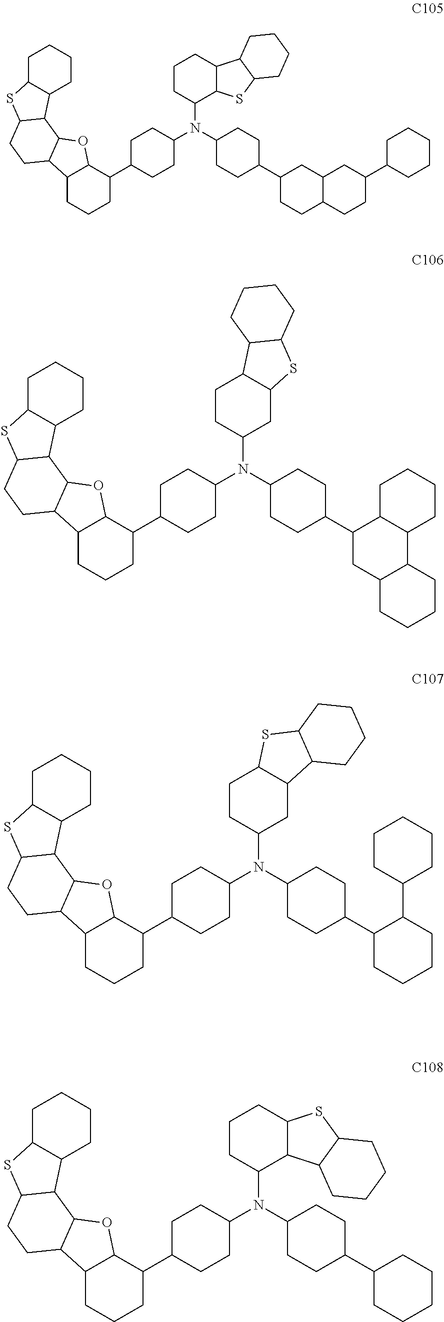 [SMILES: C1CCC(C2CCC3CCC(C4CCC(N(C5CCC(C6CCCC7C8CCC9SC%10CCCCC%10C9C8OC67)CC5)C5CCCC6C7CCCCC7SC65)CC4)CC3C2)CC1 ; C1CCC2C(C1)CC(C1CCC(N(C3CCC(C4CCCC5C6CCC7SC8CCCCC8C7C6OC45)CC3)C3CCC4C(C3)SC3CCCCC34)CC1)C1CCCCC21 ; C1CCC(C2CCCCC2C2CCC(N(C3CCC(C4CCCC5C6CCC7SC8CCCCC8C7C6OC45)CC3)C3CCC4SC5CCCCC5C4C3)CC2)CC1 ; C1CCC(C2CCC(N(C3CCC(C4CCCC5C6CCC7SC8CCCCC8C7C6OC45)CC3)C3CCCC4SC5CCCCC5C43)CC2)CC1]

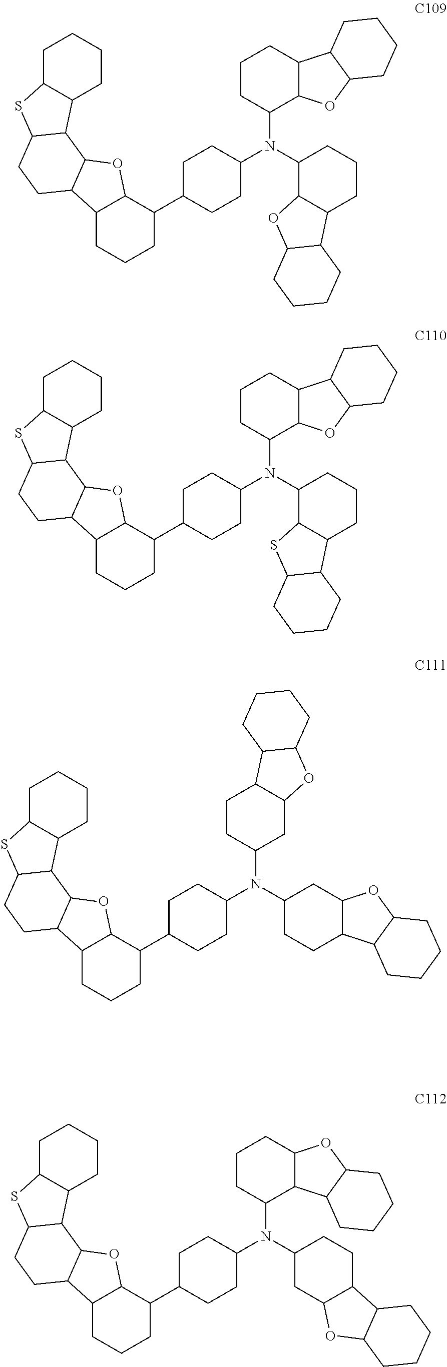 [SMILES: C1CCC2C(C1)OC1C2CCCC1N(C1CCC(C2CCCC3C4CCC5SC6CCCCC6C5C4OC23)CC1)C1CCCC2C3CCCCC3OC21 ; C1CCC2C(C1)OC1C2CCCC1N(C1CCC(C2CCCC3C4CCC5SC6CCCCC6C5C4OC23)CC1)C1CCCC2C3CCCCC3SC21 ; C1CCC2C(C1)OC1CC(N(C3CCC(C4CCCC5C6CCC7SC8CCCCC8C7C6OC45)CC3)C3CCC4C(C3)OC3CCCCC34)CCC12 ; C1CCC2C(C1)OC1CC(N(C3CCC(C4CCCC5C6CCC7SC8CCCCC8C7C6OC45)CC3)C3CCCC4OC5CCCCC5C43)CCC12]

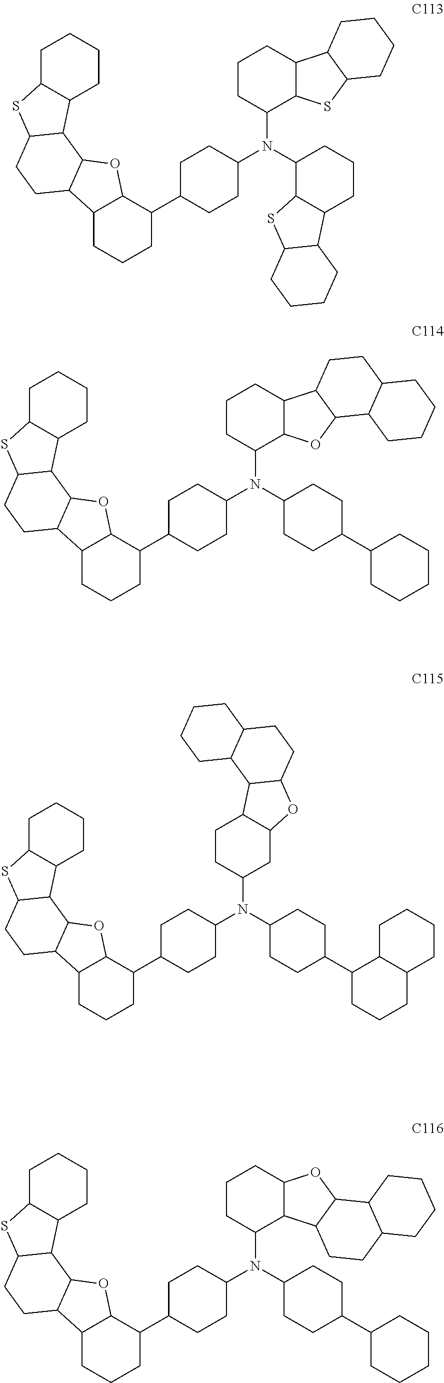 [SMILES: C1CCC2C(C1)SC1C2CCCC1N(C1CCC(C2CCCC3C4CCC5SC6CCCCC6C5C4OC23)CC1)C1CCCC2C3CCCCC3SC21 ; C1CCC(C2CCC(N(C3CCC(C4CCCC5C6CCC7SC8CCCCC8C7C6OC45)CC3)C3CCCC4C5CCC6CCCCC6C5OC43)CC2)CC1 ; C1CCC2C(C1)CCCC2C1CCC(N(C2CCC(C3CCCC4C5CCC6SC7CCCCC7C6C5OC34)CC2)C2CCC3C(C2)OC2CCC4CCCCC4C23)CC1 ; C1CCC(C2CCC(N(C3CCC(C4CCCC5C6CCC7SC8CCCCC8C7C6OC45)CC3)C3CCCC4OC5C6CCCCC6CCC5C43)CC2)CC1]

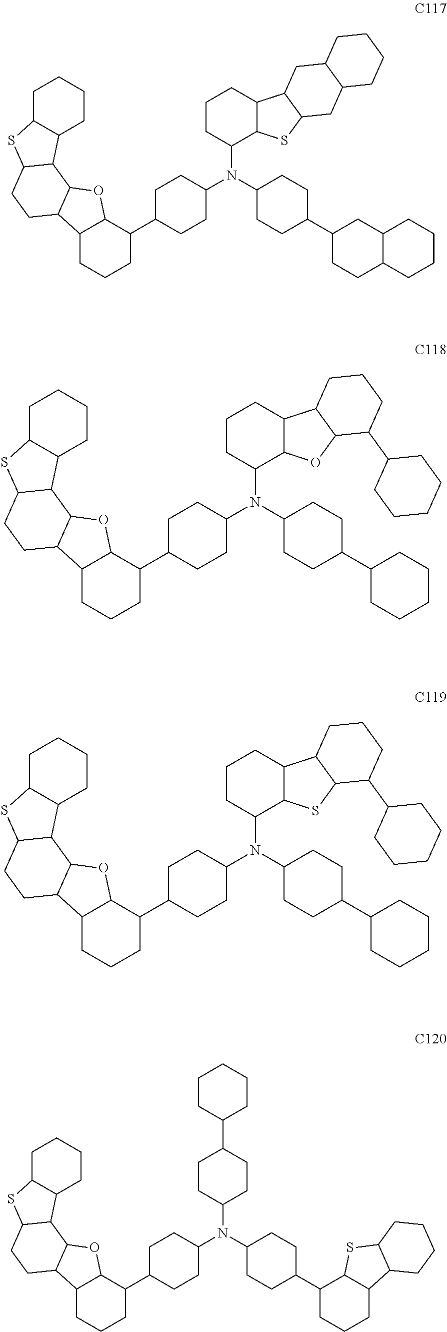 [SMILES: C1CCC2CC(C3CCC(N(C4CCC(C5CCCC6C7CCC8SC9CCCCC9C8C7OC56)CC4)C4CCCC5C6CC7CCCCC7CC6SC54)CC3)CCC2C1 ; C1CCC(C2CCC(N(C3CCC(C4CCCC5C6CCC7SC8CCCCC8C7C6OC45)CC3)C3CCCC4C5CCCC(C6CCCCC6)C5OC43)CC2)CC1 ; C1CCC(C2CCC(N(C3CCC(C4CCCC5C6CCC7SC8CCCCC8C7C6OC45)CC3)C3CCCC4C5CCCC(C6CCCCC6)C5SC43)CC2)CC1 ; C1CCC(C2CCC(N(C3CCC(C4CCCC5C6CCC7SC8CCCCC8C7C6OC45)CC3)C3CCC(C4CCCC5C6CCCCC6SC45)CC3)CC2)CC1]

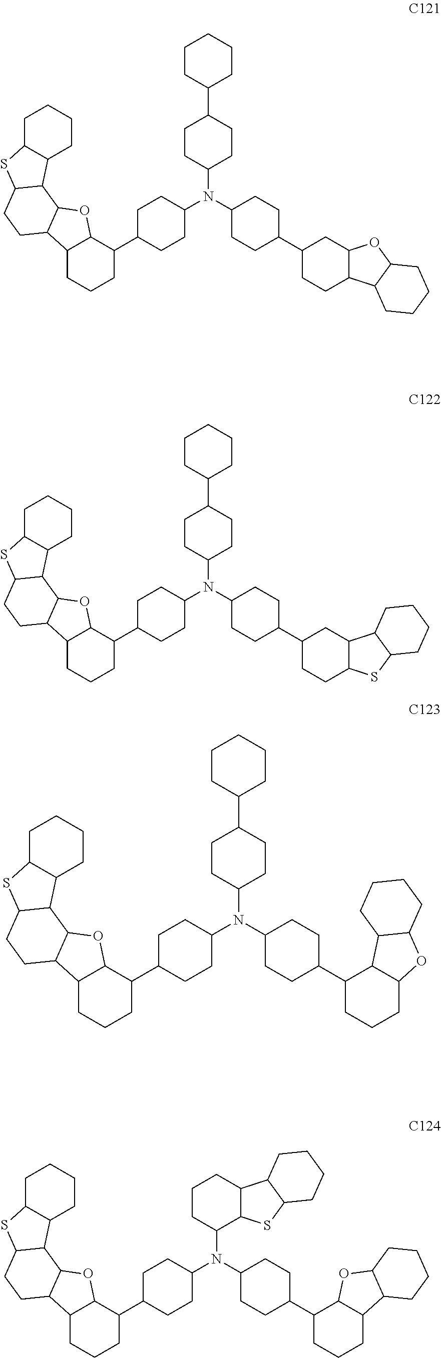 [SMILES: C1CCC(C2CCC(N(C3CCC(C4CCC5C(C4)OC4CCCCC45)CC3)C3CCC(C4CCCC5C6CCC7SC8CCCCC8C7C6OC45)CC3)CC2)CC1 ; C1CCC(C2CCC(N(C3CCC(C4CCC5SC6CCCCC6C5C4)CC3)C3CCC(C4CCCC5C6CCC7SC8CCCCC8C7C6OC45)CC3)CC2)CC1 ; C1CCC(C2CCC(N(C3CCC(C4CCCC5C6CCC7SC8CCCCC8C7C6OC45)CC3)C3CCC(C4CCCC5OC6CCCCC6C54)CC3)CC2)CC1 ; C1CCC2C(C1)OC1C(C3CCC(N(C4CCC(C5CCCC6C7CCC8SC9CCCCC9C8C7OC56)CC4)C4CCCC5C6CCCCC6SC54)CC3)CCCC21]

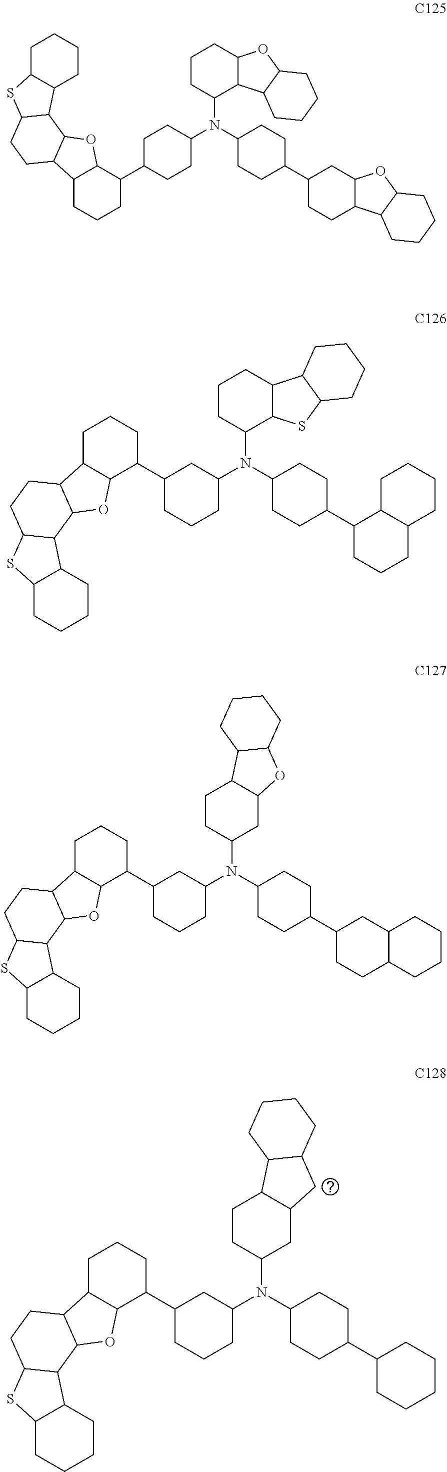 [SMILES: C1CCC2C(C1)OC1CC(C3CCC(N(C4CCC(C5CCCC6C7CCC8SC9CCCCC9C8C7OC56)CC4)C4CCCC5OC6CCCCC6C54)CC3)CCC12 ; C1CCC2C(C1)CCCC2C1CCC(N(C2CCCC(C3CCCC4C5CCC6SC7CCCCC7C6C5OC34)C2)C2CCCC3C4CCCCC4SC32)CC1 ; C1CCC2CC(C3CCC(N(C4CCCC(C5CCCC6C7CCC8SC9CCCCC9C8C7OC56)C4)C4CCC5C(C4)OC4CCCCC45)CC3)CCC2C1 ; C1CCC(C2CCC(N(C3CCC4C(CC5CCCCC54)C3)C3CCCC(C4CCCC5C6CCC7SC8CCCCC8C7C6OC45)C3)CC2)CC1]

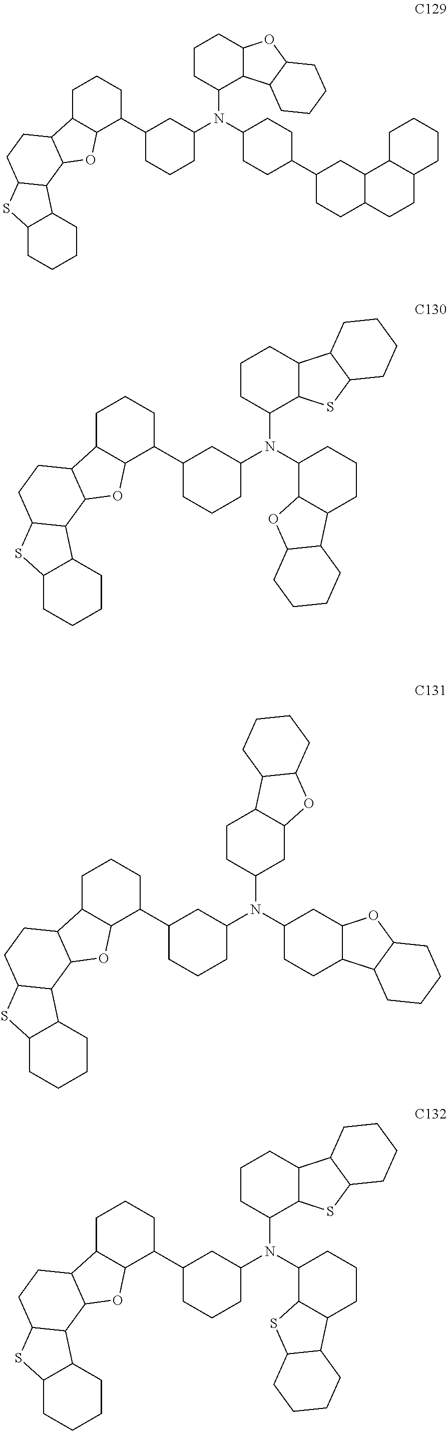 [SMILES: C1CCC2C(C1)CCC1CCC(C3CCC(N(C4CCCC(C5CCCC6C7CCC8SC9CCCCC9C8C7OC56)C4)C4CCCC5OC6CCCCC6C54)CC3)CC12 ; C1CCC2C(C1)OC1C2CCCC1N(C1CCCC(C2CCCC3C4CCC5SC6CCCCC6C5C4OC23)C1)C1CCCC2C3CCCCC3SC21 ; C1CCC2C(C1)OC1CC(N(C3CCCC(C4CCCC5C6CCC7SC8CCCCC8C7C6OC45)C3)C3CCC4C(C3)OC3CCCCC34)CCC12 ; C1CCC2C(C1)SC1C2CCCC1N(C1CCCC(C2CCCC3C4CCC5SC6CCCCC6C5C4OC23)C1)C1CCCC2C3CCCCC3SC21]

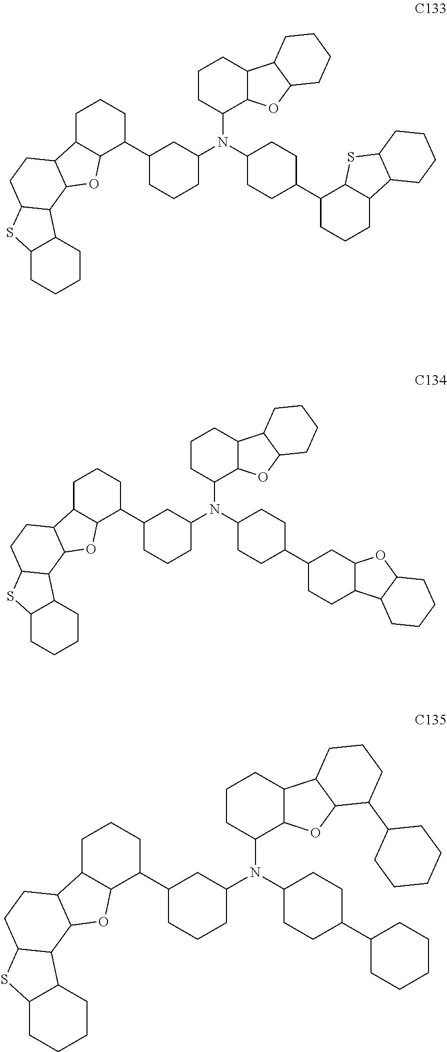 [SMILES: C1CCC2C(C1)OC1C2CCCC1N(C1CCC(C2CCCC3C4CCCCC4SC23)CC1)C1CCCC(C2CCCC3C4CCC5SC6CCCCC6C5C4OC23)C1 ; C1CCC2C(C1)OC1CC(C3CCC(N(C4CCCC(C5CCCC6C7CCC8SC9CCCCC9C8C7OC56)C4)C4CCCC5C6CCCCC6OC54)CC3)CCC12 ; C1CCC(C2CCC(N(C3CCCC(C4CCCC5C6CCC7SC8CCCCC8C7C6OC45)C3)C3CCCC4C5CCCC(C6CCCCC6)C5OC43)CC2)CC1]

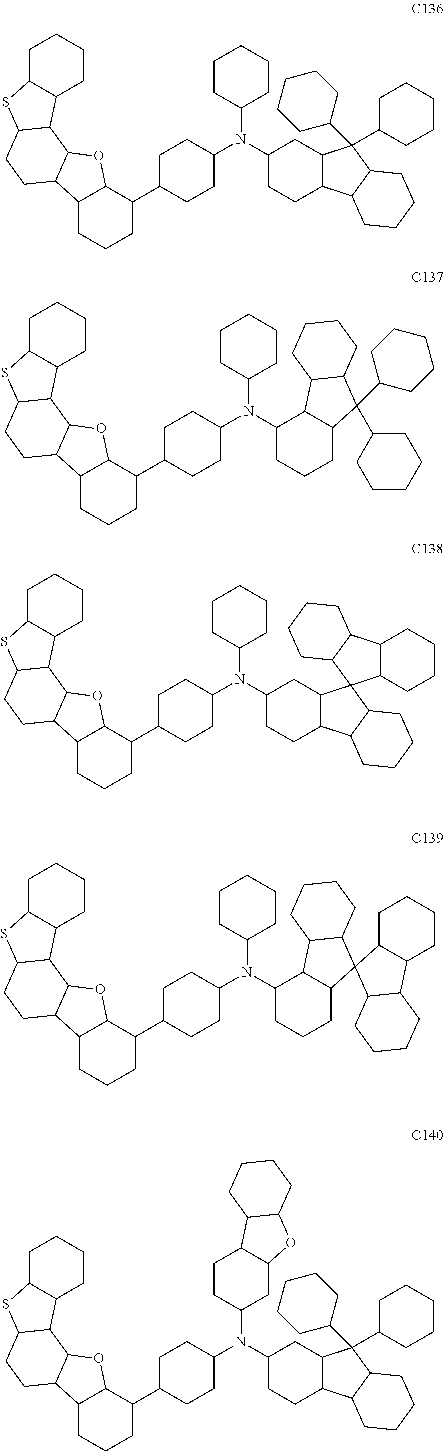 [SMILES: C1CCC(N(C2CCC(C3CCCC4C5CCC6SC7CCCCC7C6C5OC34)CC2)C2CCC3C4CCCCC4C(C4CCCCC4)(C4CCCCC4)C3C2)CC1 ; C1CCC(N(C2CCC(C3CCCC4C5CCC6SC7CCCCC7C6C5OC34)CC2)C2CCCC3C2C2CCCCC2C3(C2CCCCC2)C2CCCCC2)CC1 ; C1CCC(N(C2CCC(C3CCCC4C5CCC6SC7CCCCC7C6C5OC34)CC2)C2CCC3C4CCCCC4C4(C5CCCCC5C5CCCCC54)C3C2)CC1 ; C1CCC(N(C2CCC(C3CCCC4C5CCC6SC7CCCCC7C6C5OC34)CC2)C2CCCC3C2C2CCCCC2C32C3CCCCC3C3CCCCC32)CC1 ; C1CCC(C2(C3CCCCC3)C3CCCCC3C3CCC(N(C4CCC(C5CCCC6C7CCC8SC9CCCCC9C8C7OC56)CC4)C4CCC5C(C4)OC4CCCCC45)CC32)CC1]

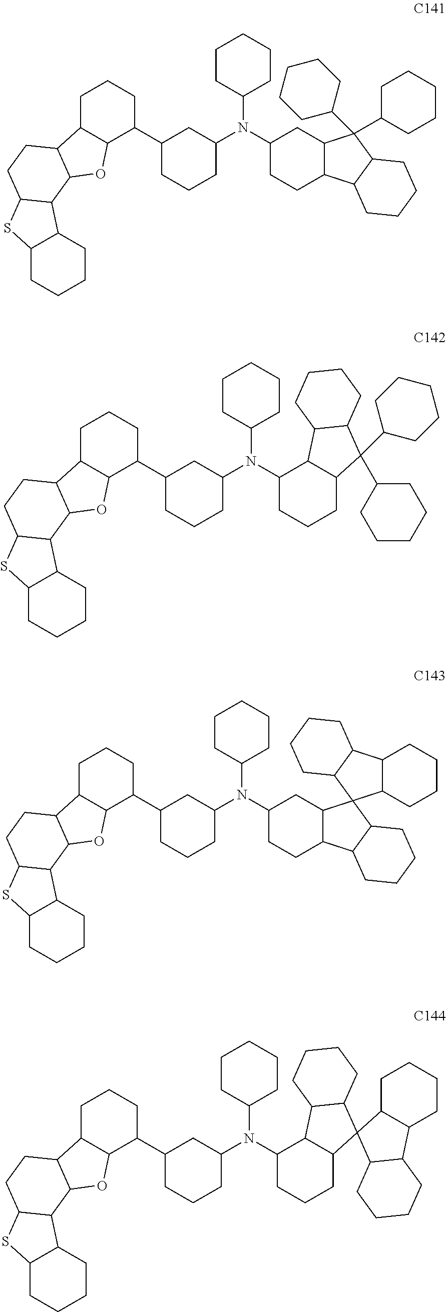 [SMILES: C1CCC(N(C2CCCC(C3CCCC4C5CCC6SC7CCCCC7C6C5OC34)C2)C2CCC3C4CCCCC4C(C4CCCCC4)(C4CCCCC4)C3C2)CC1 ; C1CCC(N(C2CCCC(C3CCCC4C5CCC6SC7CCCCC7C6C5OC34)C2)C2CCCC3C2C2CCCCC2C3(C2CCCCC2)C2CCCCC2)CC1 ; C1CCC(N(C2CCCC(C3CCCC4C5CCC6SC7CCCCC7C6C5OC34)C2)C2CCC3C4CCCCC4C4(C5CCCCC5C5CCCCC54)C3C2)CC1 ; C1CCC(N(C2CCCC(C3CCCC4C5CCC6SC7CCCCC7C6C5OC34)C2)C2CCCC3C2C2CCCCC2C32C3CCCCC3C3CCCCC32)CC1]

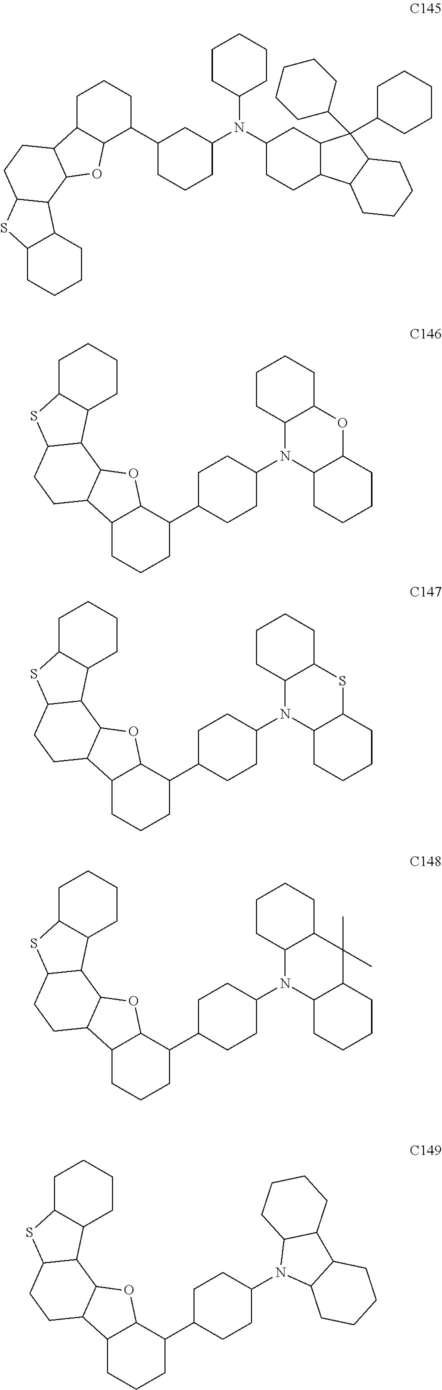 [SMILES: C1CCC(N(C2CCCC(C3CCCC4C5CCC6SC7CCCCC7C6C5OC34)C2)C2CCC3C4CCCCC4C(C4CCCCC4)(C4CCCCC4)C3C2)CC1 ; C1CCC2C(C1)OC1CCCCC1N2C1CCC(C2CCCC3C4CCC5SC6CCCCC6C5C4OC23)CC1 ; C1CCC2C(C1)SC1CCC3C4CCCC(C5CCC(N6C7CCCCC7SC7CCCCC76)CC5)C4OC3C12 ; CC1(C)C2CCCCC2N(C2CCC(C3CCCC4C5CCC6SC7CCCCC7C6C5OC34)CC2)C2CCCCC21 ; C1CCC2C(C1)SC1CCC3C4CCCC(C5CCC(N6C7CCCCC7C7CCCCC76)CC5)C4OC3C12]

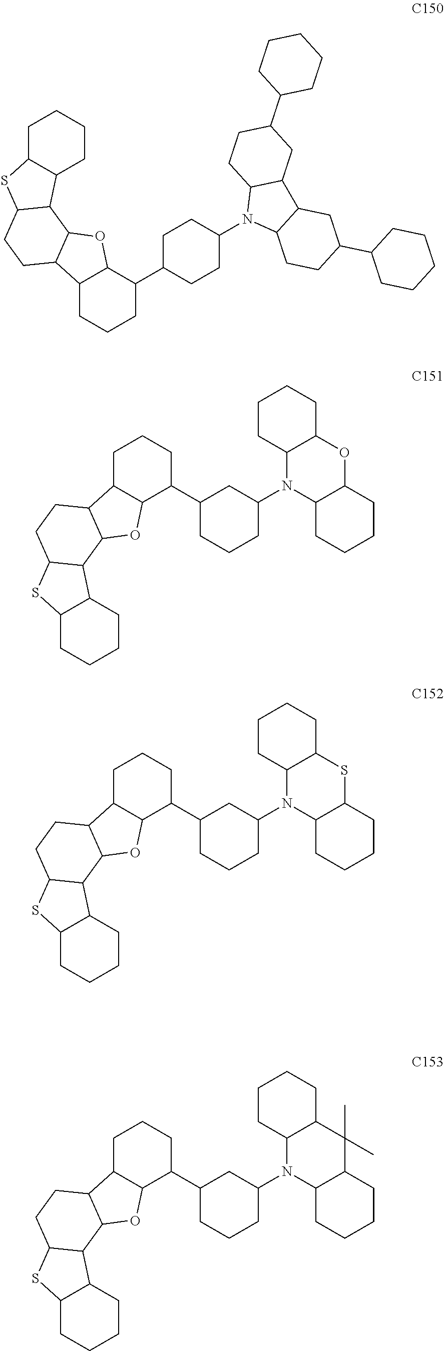 [SMILES: C1CCC(C2CCC3C(C2)C2CC(C4CCCCC4)CCC2N3C2CCC(C3CCCC4C5CCC6SC7CCCCC7C6C5OC34)CC2)CC1 ; C1CCC2C(C1)OC1CCCCC1N2C1CCCC(C2CCCC3C4CCC5SC6CCCCC6C5C4OC23)C1 ; C1CCC2C(C1)SC1CCC3C4CCCC(C5CCCC(N6C7CCCCC7SC7CCCCC76)C5)C4OC3C12 ; CC1(C)C2CCCCC2N(C2CCCC(C3CCCC4C5CCC6SC7CCCCC7C6C5OC34)C2)C2CCCCC21]

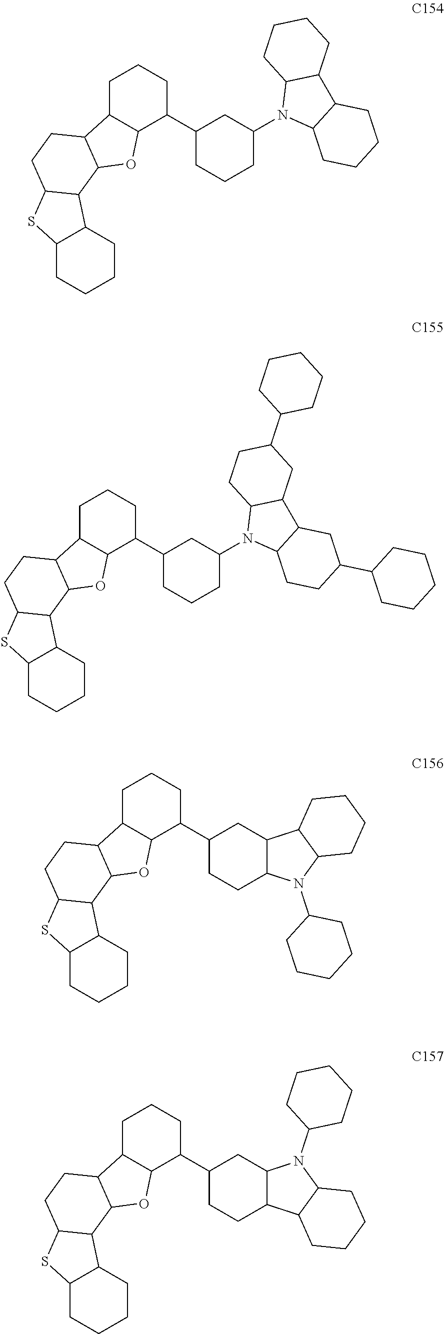 [SMILES: C1CCC2C(C1)SC1CCC3C4CCCC(C5CCCC(N6C7CCCCC7C7CCCCC76)C5)C4OC3C12 ; C1CCC(C2CCC3C(C2)C2CC(C4CCCCC4)CCC2N3C2CCCC(C3CCCC4C5CCC6SC7CCCCC7C6C5OC34)C2)CC1 ; C1CCC(N2C3CCCCC3C3CC(C4CCCC5C6CCC7SC8CCCCC8C7C6OC45)CCC32)CC1 ; C1CCC(N2C3CCCCC3C3CCC(C4CCCC5C6CCC7SC8CCCCC8C7C6OC45)CC32)CC1]

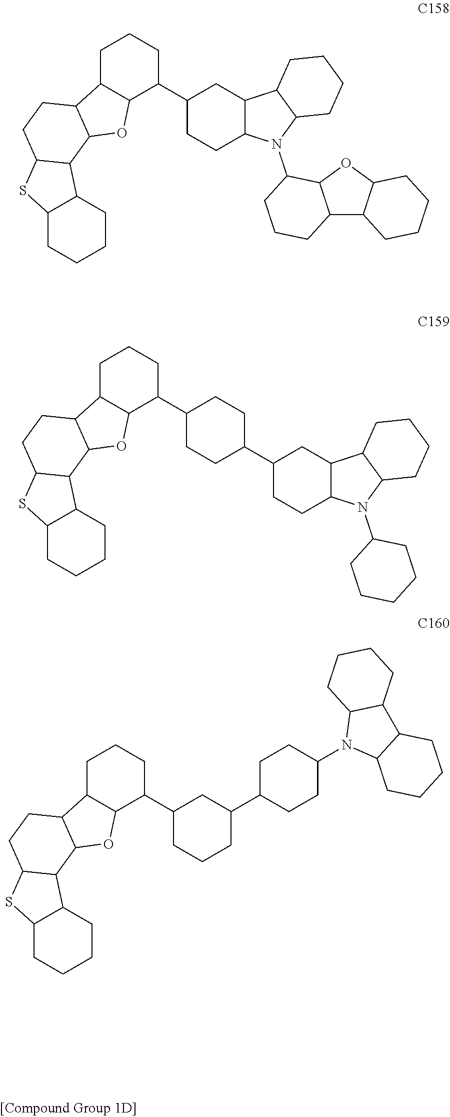 [SMILES: C1CCC2C(C1)OC1C2CCCC1N1C2CCCCC2C2CC(C3CCCC4C5CCC6SC7CCCCC7C6C5OC34)CCC21 ; C1CCC(N2C3CCCCC3C3CC(C4CCC(C5CCCC6C7CCC8SC9CCCCC9C8C7OC56)CC4)CCC32)CC1 ; C1CC(C2CCC(N3C4CCCCC4C4CCCCC43)CC2)CC(C2CCCC3C4CCC5SC6CCCCC6C5C4OC23)C1]

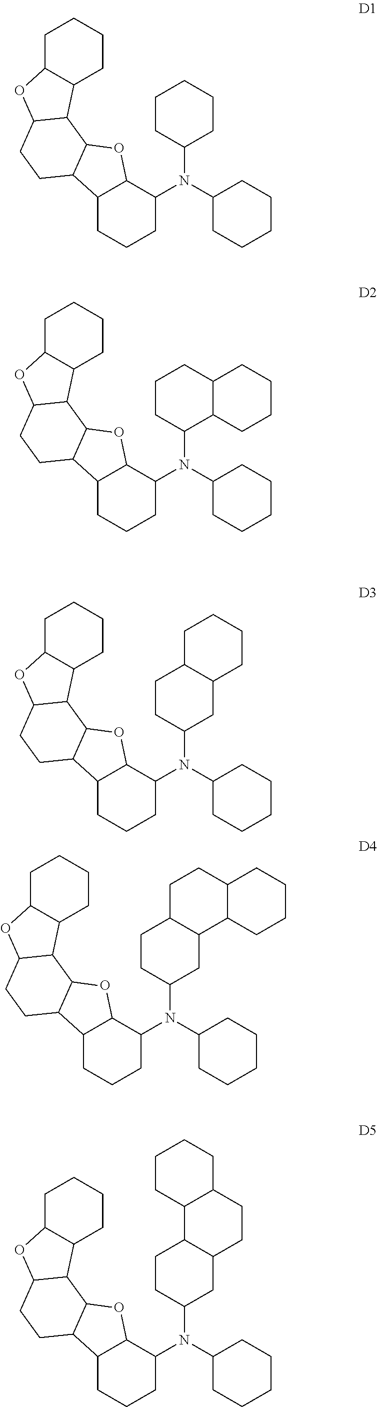

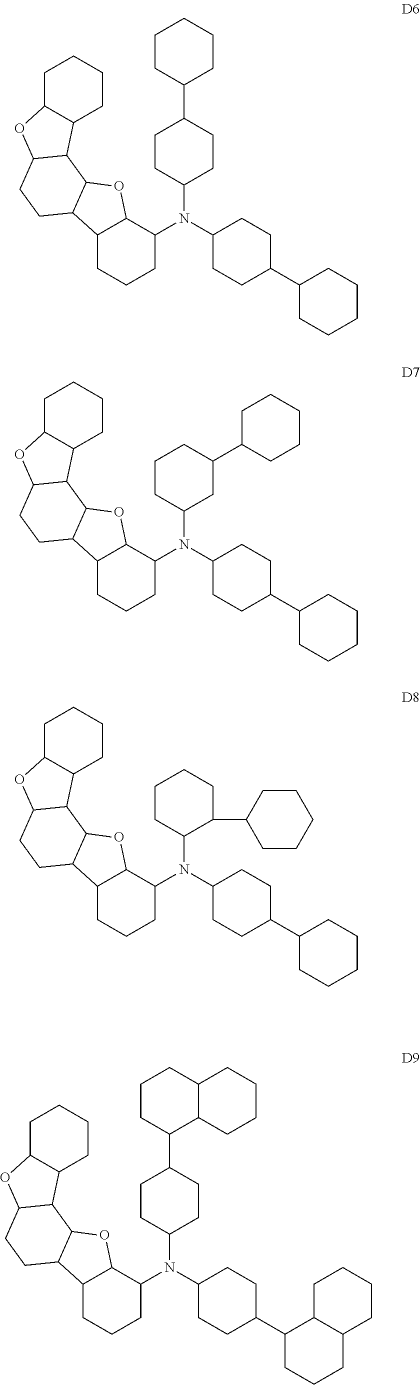 [SMILES: C1CCC(C2CCC(N(C3CCC(C4CCCCC4)CC3)C3CCCC4C5CCC6OC7CCCCC7C6C5OC43)CC2)CC1 ; C1CCC(C2CCC(N(C3CCCC(C4CCCCC4)C3)C3CCCC4C5CCC6OC7CCCCC7C6C5OC43)CC2)CC1 ; C1CCC(C2CCC(N(C3CCCCC3C3CCCCC3)C3CCCC4C5CCC6OC7CCCCC7C6C5OC43)CC2)CC1 ; C1CCC2C(C1)CCCC2C1CCC(N(C2CCC(C3CCCC4CCCCC43)CC2)C2CCCC3C4CCC5OC6CCCCC6C5C4OC32)CC1]

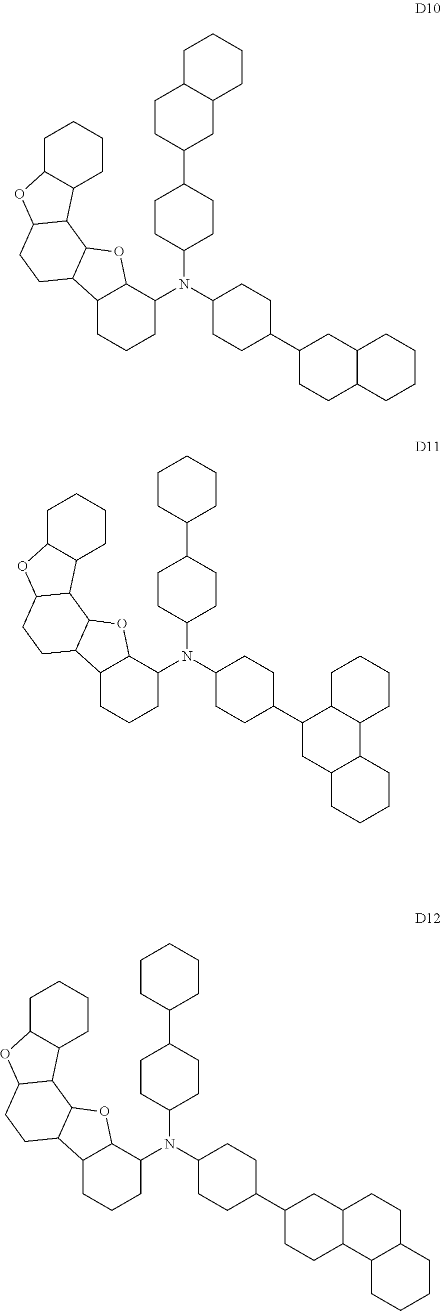 [SMILES: C1CCC2CC(C3CCC(N(C4CCC(C5CCC6CCCCC6C5)CC4)C4CCCC5C6CCC7OC8CCCCC8C7C6OC54)CC3)CCC2C1 ; C1CCC(C2CCC(N(C3CCC(C4CC5CCCCC5C5CCCCC45)CC3)C3CCCC4C5CCC6OC7CCCCC7C6C5OC43)CC2)CC1 ; C1CCC(C2CCC(N(C3CCC(C4CCC5C(CCC6CCCCC65)C4)CC3)C3CCCC4C5CCC6OC7CCCCC7C6C5OC43)CC2)CC1]

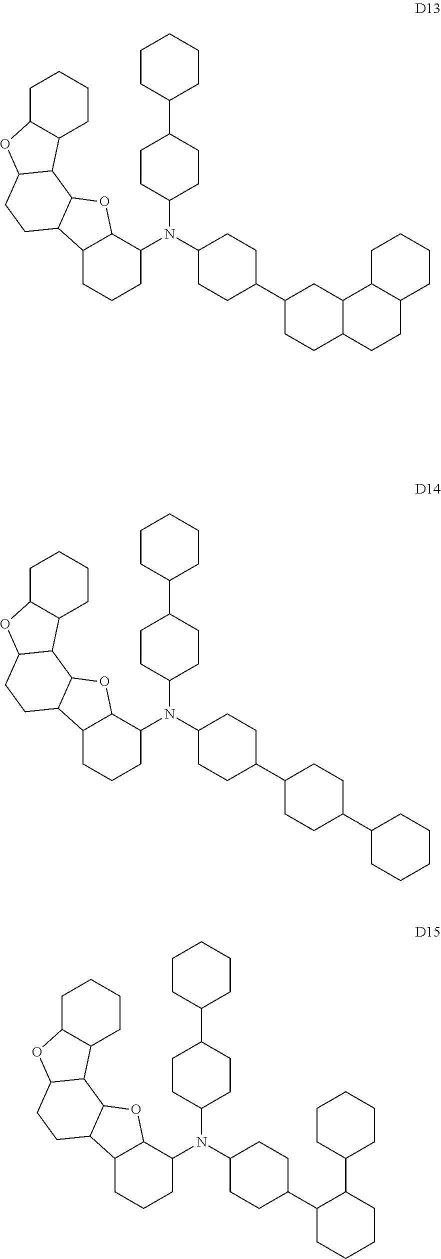 [SMILES: C1CCC(C2CCC(N(C3CCC(C4CCC5CCC6CCCCC6C5C4)CC3)C3CCCC4C5CCC6OC7CCCCC7C6C5OC43)CC2)CC1 ; C1CCC(C2CCC(C3CCC(N(C4CCC(C5CCCCC5)CC4)C4CCCC5C6CCC7OC8CCCCC8C7C6OC54)CC3)CC2)CC1 ; C1CCC(C2CCC(N(C3CCC(C4CCCCC4C4CCCCC4)CC3)C3CCCC4C5CCC6OC7CCCCC7C6C5OC43)CC2)CC1]

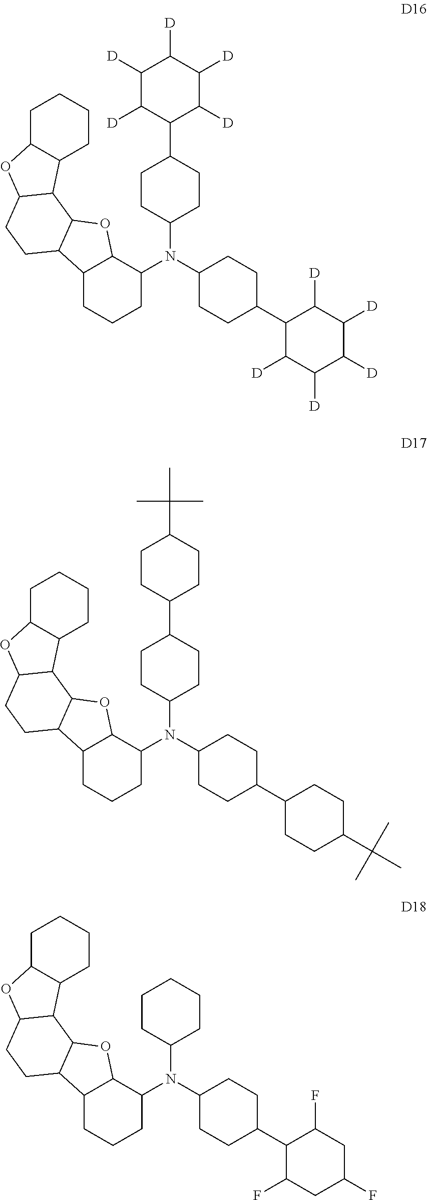 [SMILES: [2H]C1C([2H])C([2H])C(C2CCC(N(C3CCC(C4C([2H])C([2H])C([2H])C([2H])C4[2H])CC3)C3CCCC4C5CCC6OC7CCCCC7C6C5OC43)CC2)C([2H])C1[2H] ; CC(C)(C)C1CCC(C2CCC(N(C3CCC(C4CCC(C(C)(C)C)CC4)CC3)C3CCCC4C5CCC6OC7CCCCC7C6C5OC43)CC2)CC1 ; FC1CC(F)C(C2CCC(N(C3CCCCC3)C3CCCC4C5CCC6OC7CCCCC7C6C5OC43)CC2)C(F)C1]

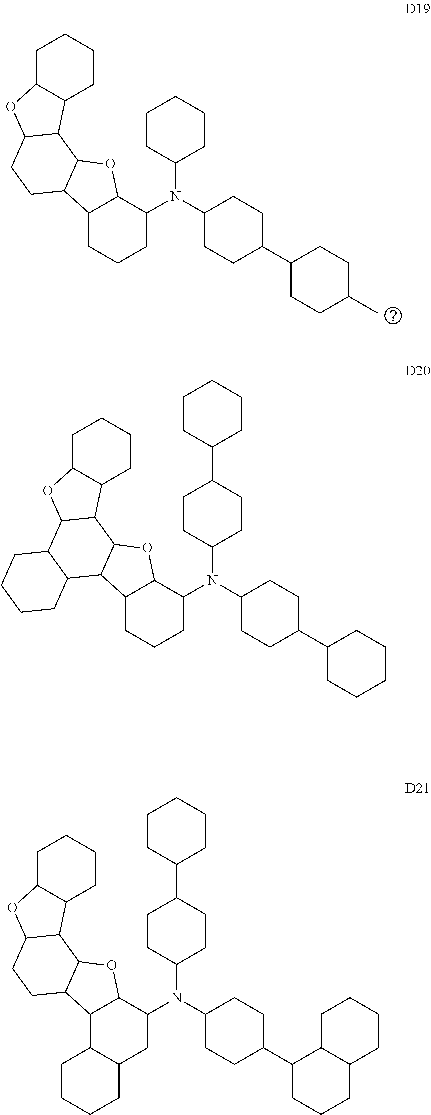 [SMILES: CC1CCC(C2CCC(N(C3CCCCC3)C3CCCC4C5CCC6OC7CCCCC7C6C5OC43)CC2)CC1 ; C1CCC(C2CCC(N(C3CCC(C4CCCCC4)CC3)C3CCCC4C3OC3C4C4CCCCC4C4OC5CCCCC5C43)CC2)CC1 ; C1CCC(C2CCC(N(C3CCC(C4CCCC5CCCCC54)CC3)C3CC4CCCCC4C4C5CCC6OC7CCCCC7C6C5OC43)CC2)CC1]

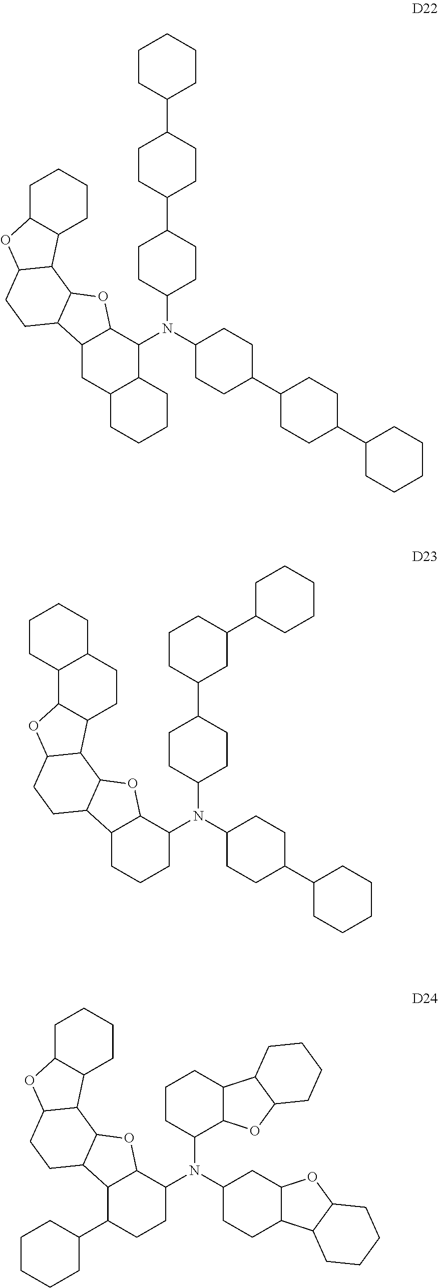 [SMILES: C1CCC(C2CCC(C3CCC(N(C4CCC(C5CCC(C6CCCCC6)CC5)CC4)C4C5CCCCC5CC5C6CCC7OC8CCCCC8C7C6OC54)CC3)CC2)CC1 ; C1CCC(C2CCC(N(C3CCC(C4CCCC(C5CCCCC5)C4)CC3)C3CCCC4C5CCC6OC7C8CCCCC8CCC7C6C5OC43)CC2)CC1 ; C1CCC(C2CCC(N(C3CCC4C(C3)OC3CCCCC34)C3CCCC4C5CCCCC5OC43)C3OC4C(CCC5OC6CCCCC6C54)C23)CC1]

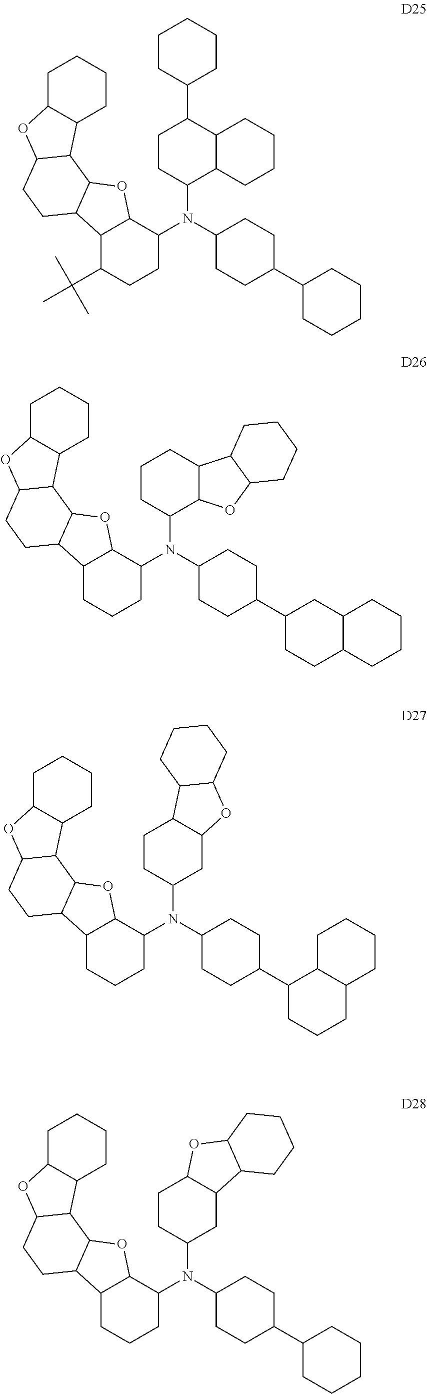 [SMILES: CC(C)(C)C1CCC(N(C2CCC(C3CCCCC3)CC2)C2CCC(C3CCCCC3)C3CCCCC32)C2OC3C(CCC4OC5CCCCC5C43)C21 ; C1CCC2CC(C3CCC(N(C4CCCC5C6CCCCC6OC54)C4CCCC5C6CCC7OC8CCCCC8C7C6OC54)CC3)CCC2C1 ; C1CCC2C(C1)CCCC2C1CCC(N(C2CCC3C(C2)OC2CCCCC23)C2CCCC3C4CCC5OC6CCCCC6C5C4OC32)CC1 ; C1CCC(C2CCC(N(C3CCC4OC5CCCCC5C4C3)C3CCCC4C5CCC6OC7CCCCC7C6C5OC43)CC2)CC1]

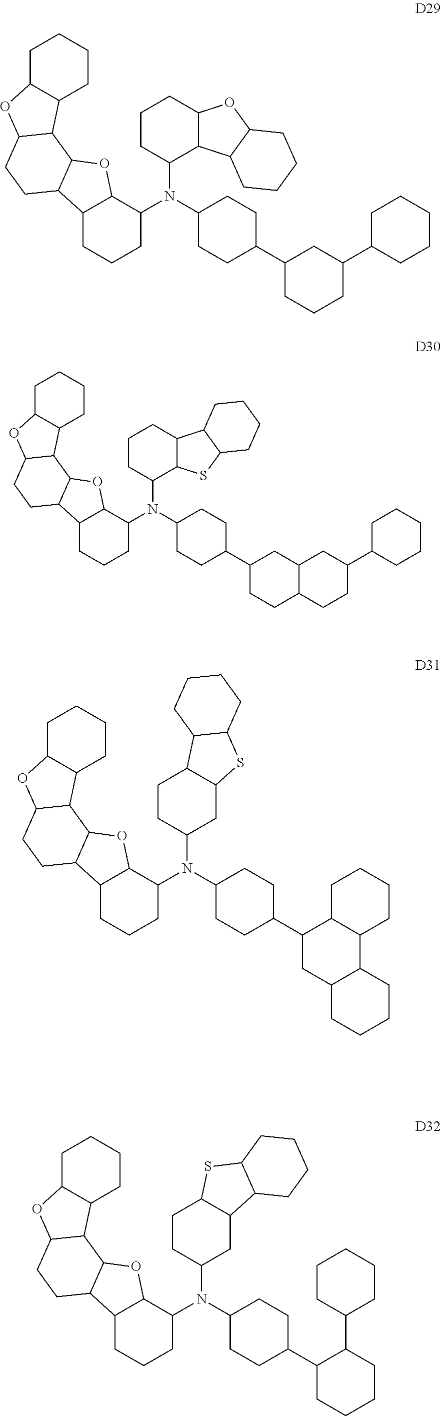 [SMILES: C1CCC(C2CCCC(C3CCC(N(C4CCCC5C6CCC7OC8CCCCC8C7C6OC54)C4CCCC5OC6CCCCC6C54)CC3)C2)CC1 ; C1CCC(C2CCC3CCC(C4CCC(N(C5CCCC6C7CCC8OC9CCCCC9C8C7OC65)C5CCCC6C7CCCCC7SC65)CC4)CC3C2)CC1 ; C1CCC2C(C1)CC(C1CCC(N(C3CCC4C(C3)SC3CCCCC34)C3CCCC4C5CCC6OC7CCCCC7C6C5OC43)CC1)C1CCCCC21 ; C1CCC(C2CCCCC2C2CCC(N(C3CCC4SC5CCCCC5C4C3)C3CCCC4C5CCC6OC7CCCCC7C6C5OC43)CC2)CC1]

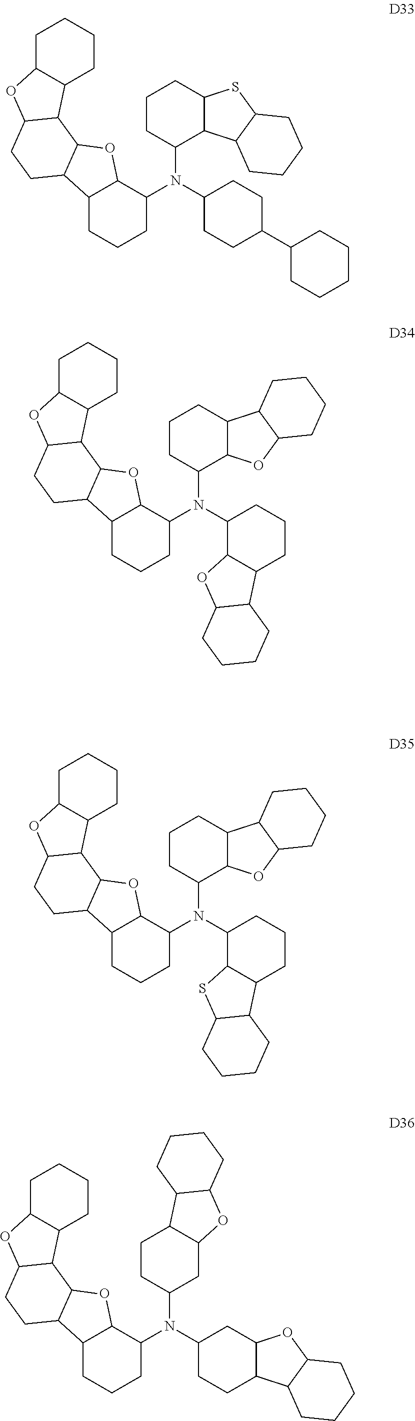 [SMILES: C1CCC(C2CCC(N(C3CCCC4C5CCC6OC7CCCCC7C6C5OC43)C3CCCC4SC5CCCCC5C43)CC2)CC1 ; C1CCC2C(C1)OC1C2CCCC1N(C1CCCC2C3CCCCC3OC21)C1CCCC2C3CCC4OC5CCCCC5C4C3OC21 ; C1CCC2C(C1)OC1C2CCCC1N(C1CCCC2C3CCC4OC5CCCCC5C4C3OC21)C1CCCC2C3CCCCC3SC21 ; C1CCC2C(C1)OC1CC(N(C3CCC4C(C3)OC3CCCCC34)C3CCCC4C5CCC6OC7CCCCC7C6C5OC43)CCC12]

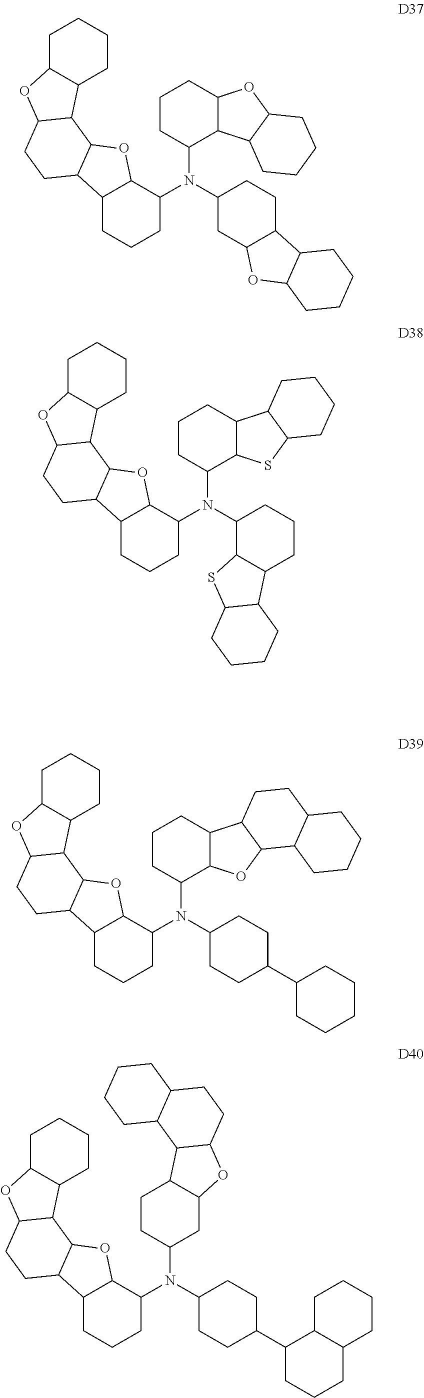 [SMILES: C1CCC2C(C1)OC1CC(N(C3CCCC4C5CCC6OC7CCCCC7C6C5OC43)C3CCCC4OC5CCCCC5C43)CCC12 ; C1CCC2C(C1)OC1CCC3C4CCCC(N(C5CCCC6C7CCCCC7SC65)C5CCCC6C7CCCCC7SC65)C4OC3C12 ; C1CCC(C2CCC(N(C3CCCC4C5CCC6CCCCC6C5OC43)C3CCCC4C5CCC6OC7CCCCC7C6C5OC43)CC2)CC1 ; C1CCC2C(C1)CCCC2C1CCC(N(C2CCC3C(C2)OC2CCC4CCCCC4C23)C2CCCC3C4CCC5OC6CCCCC6C5C4OC32)CC1]

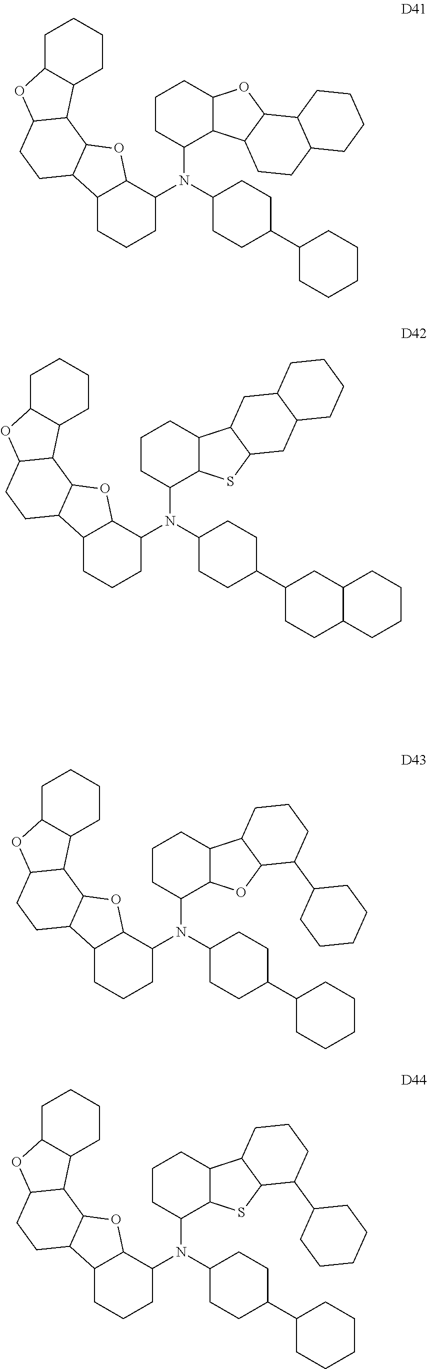 [SMILES: C1CCC(C2CCC(N(C3CCCC4C5CCC6OC7CCCCC7C6C5OC43)C3CCCC4OC5C6CCCCC6CCC5C43)CC2)CC1 ; C1CCC2CC(C3CCC(N(C4CCCC5C6CCC7OC8CCCCC8C7C6OC54)C4CCCC5C6CC7CCCCC7CC6SC54)CC3)CCC2C1 ; C1CCC(C2CCC(N(C3CCCC4C5CCCC(C6CCCCC6)C5OC43)C3CCCC4C5CCC6OC7CCCCC7C6C5OC43)CC2)CC1 ; C1CCC(C2CCC(N(C3CCCC4C5CCC6OC7CCCCC7C6C5OC43)C3CCCC4C5CCCC(C6CCCCC6)C5SC43)CC2)CC1]

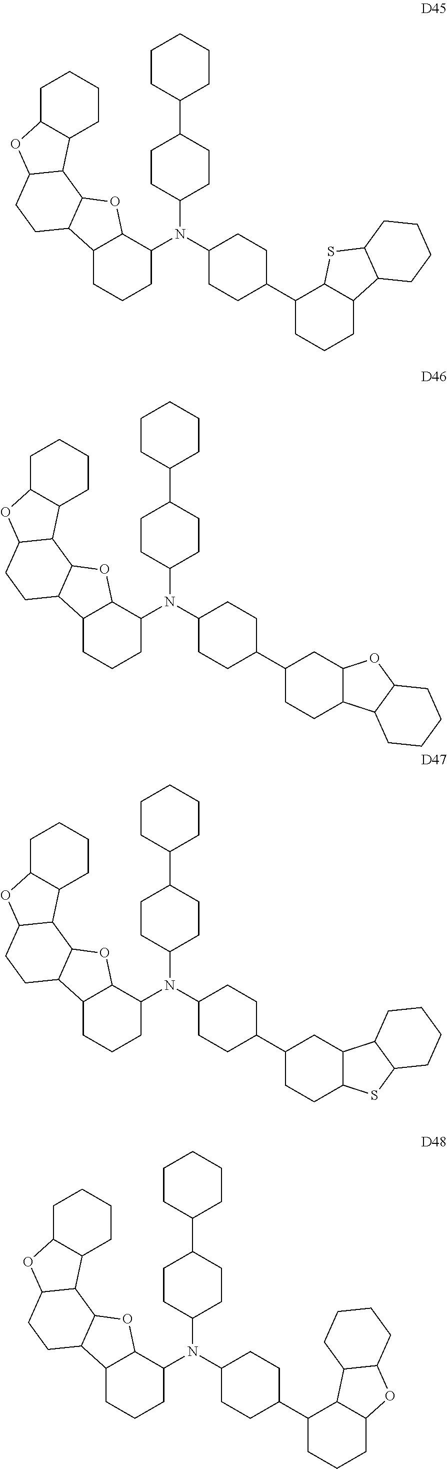 [SMILES: C1CCC(C2CCC(N(C3CCC(C4CCCC5C6CCCCC6SC45)CC3)C3CCCC4C5CCC6OC7CCCCC7C6C5OC43)CC2)CC1 ; C1CCC(C2CCC(N(C3CCC(C4CCC5C(C4)OC4CCCCC45)CC3)C3CCCC4C5CCC6OC7CCCCC7C6C5OC43)CC2)CC1 ; C1CCC(C2CCC(N(C3CCC(C4CCC5SC6CCCCC6C5C4)CC3)C3CCCC4C5CCC6OC7CCCCC7C6C5OC43)CC2)CC1 ; C1CCC(C2CCC(N(C3CCC(C4CCCC5OC6CCCCC6C54)CC3)C3CCCC4C5CCC6OC7CCCCC7C6C5OC43)CC2)CC1]

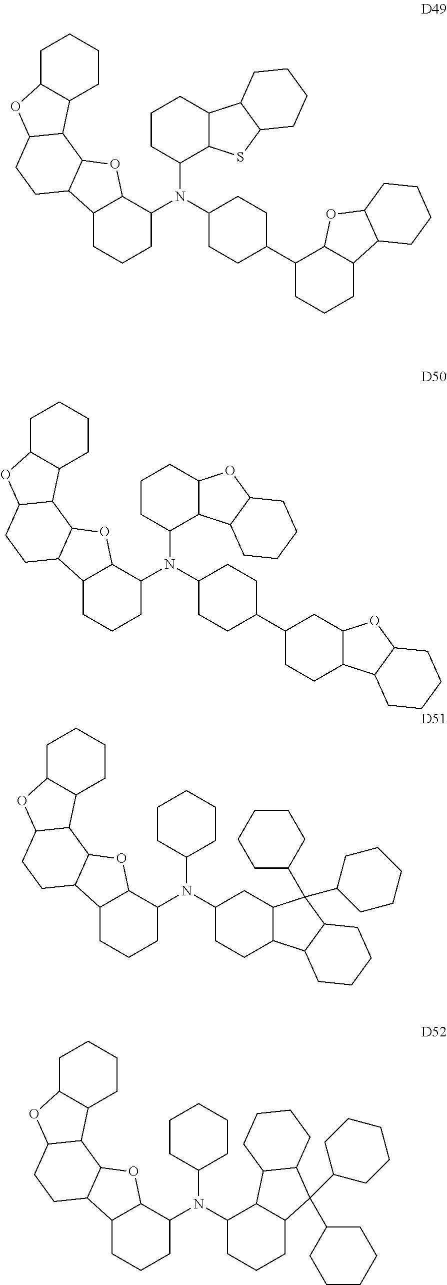 [SMILES: C1CCC2C(C1)OC1C(C3CCC(N(C4CCCC5C6CCC7OC8CCCCC8C7C6OC54)C4CCCC5C6CCCCC6SC54)CC3)CCCC21 ; C1CCC2C(C1)OC1CC(C3CCC(N(C4CCCC5C6CCC7OC8CCCCC8C7C6OC54)C4CCCC5OC6CCCCC6C54)CC3)CCC12 ; C1CCC(N(C2CCC3C4CCCCC4C(C4CCCCC4)(C4CCCCC4)C3C2)C2CCCC3C4CCC5OC6CCCCC6C5C4OC32)CC1 ; C1CCC(N(C2CCCC3C4CCC5OC6CCCCC6C5C4OC32)C2CCCC3C2C2CCCCC2C3(C2CCCCC2)C2CCCCC2)CC1]

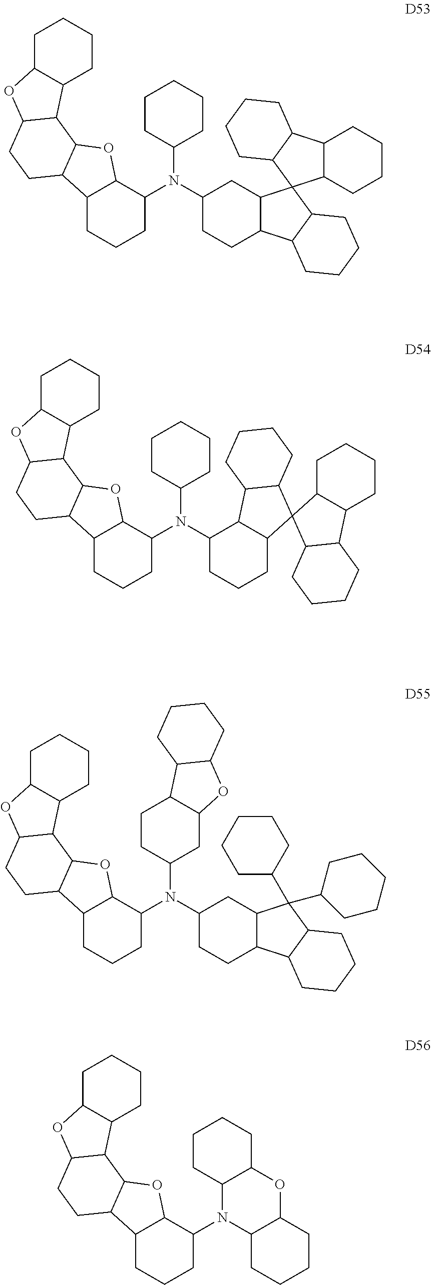

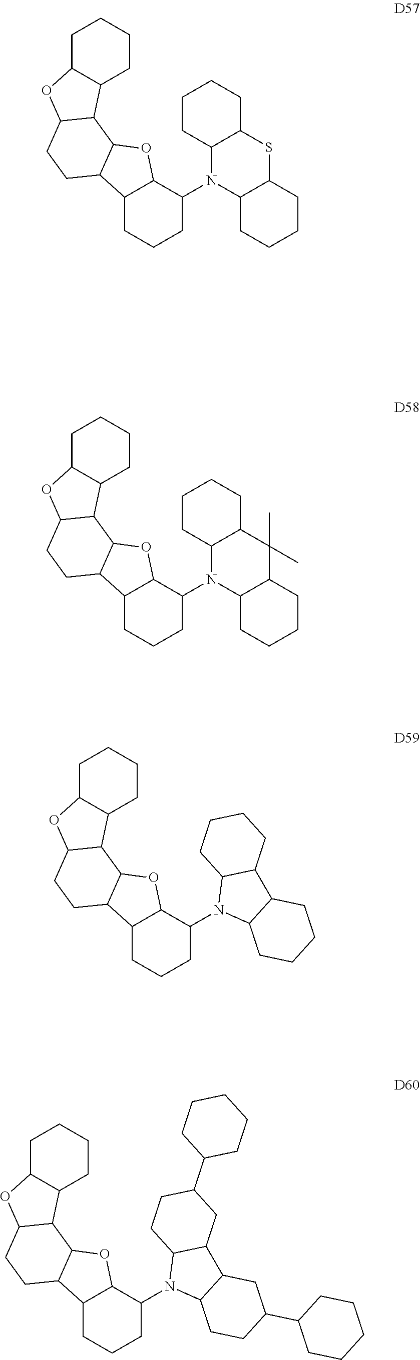

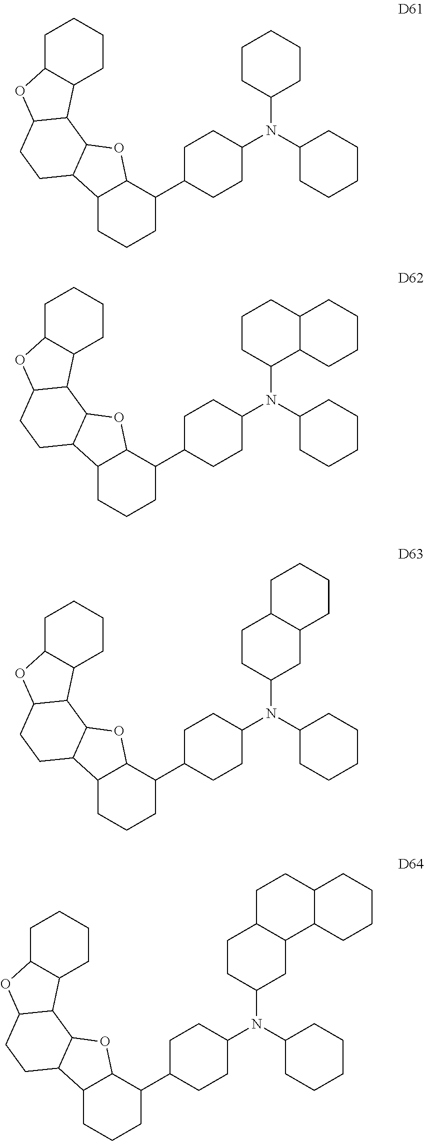 [SMILES: C1CCC(N(C2CCCCC2)C2CCC(C3CCCC4C5CCC6OC7CCCCC7C6C5OC34)CC2)CC1 ; C1CCC(N(C2CCC(C3CCCC4C5CCC6OC7CCCCC7C6C5OC34)CC2)C2CCCC3CCCCC32)CC1 ; C1CCC(N(C2CCC(C3CCCC4C5CCC6OC7CCCCC7C6C5OC34)CC2)C2CCC3CCCCC3C2)CC1 ; C1CCC(N(C2CCC(C3CCCC4C5CCC6OC7CCCCC7C6C5OC34)CC2)C2CCC3CCC4CCCCC4C3C2)CC1]

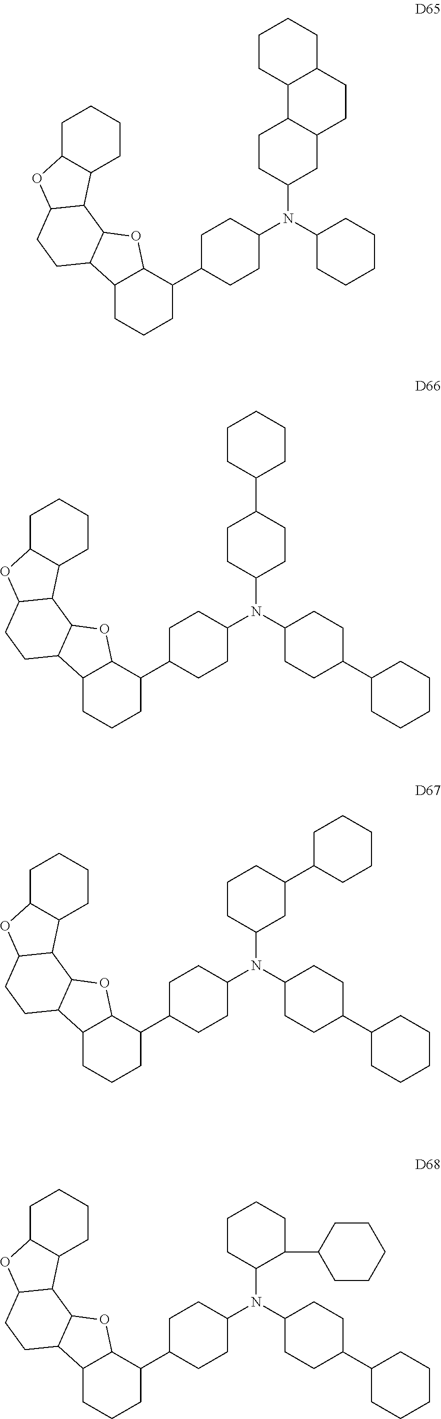 [SMILES: C1CCC(N(C2CCC(C3CCCC4C5CCC6OC7CCCCC7C6C5OC34)CC2)C2CCC3C(CCC4CCCCC43)C2)CC1 ; C1CCC(C2CCC(N(C3CCC(C4CCCCC4)CC3)C3CCC(C4CCCC5C6CCC7OC8CCCCC8C7C6OC45)CC3)CC2)CC1 ; C1CCC(C2CCC(N(C3CCC(C4CCCC5C6CCC7OC8CCCCC8C7C6OC45)CC3)C3CCCC(C4CCCCC4)C3)CC2)CC1 ; C1CCC(C2CCC(N(C3CCC(C4CCCC5C6CCC7OC8CCCCC8C7C6OC45)CC3)C3CCCCC3C3CCCCC3)CC2)CC1]

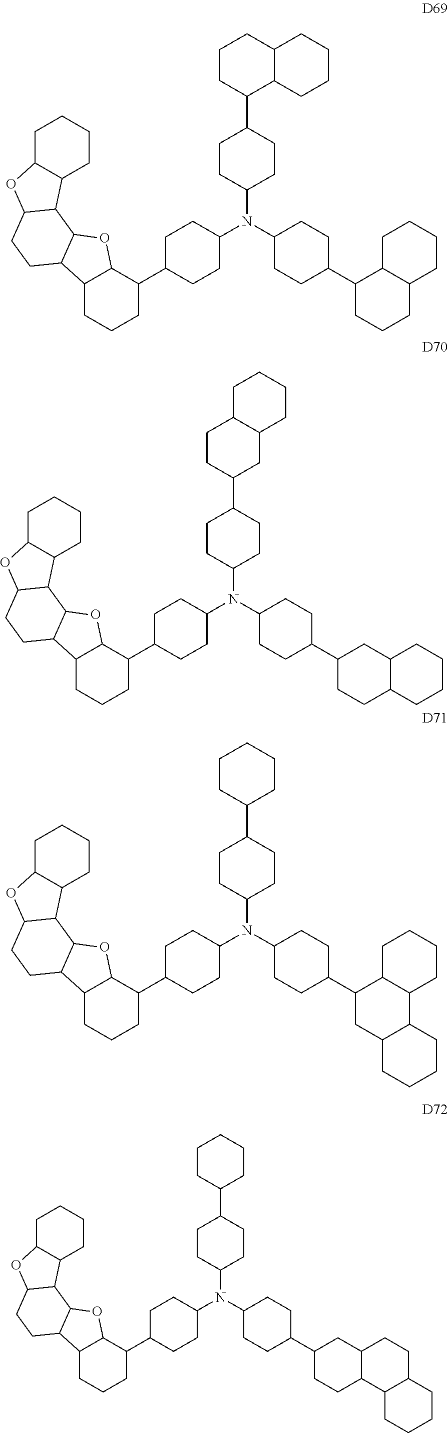 [SMILES: C1CCC2C(C1)CCCC2C1CCC(N(C2CCC(C3CCCC4CCCCC43)CC2)C2CCC(C3CCCC4C5CCC6OC7CCCCC7C6C5OC34)CC2)CC1 ; C1CCC2CC(C3CCC(N(C4CCC(C5CCC6CCCCC6C5)CC4)C4CCC(C5CCCC6C7CCC8OC9CCCCC9C8C7OC56)CC4)CC3)CCC2C1 ; C1CCC(C2CCC(N(C3CCC(C4CC5CCCCC5C5CCCCC45)CC3)C3CCC(C4CCCC5C6CCC7OC8CCCCC8C7C6OC45)CC3)CC2)CC1 ; C1CCC(C2CCC(N(C3CCC(C4CCC5C(CCC6CCCCC65)C4)CC3)C3CCC(C4CCCC5C6CCC7OC8CCCCC8C7C6OC45)CC3)CC2)CC1]

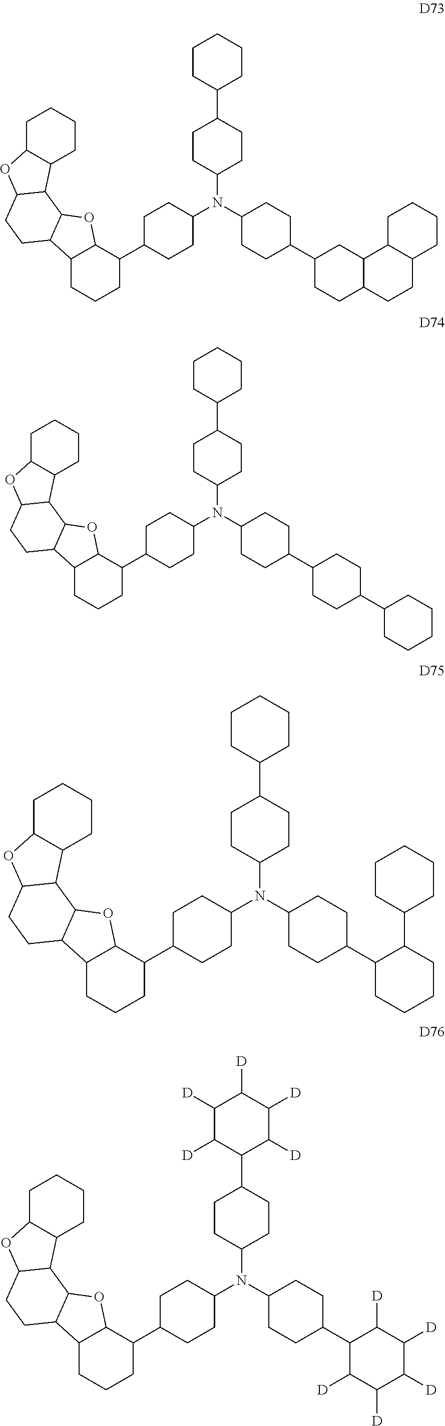 [SMILES: C1CCC(C2CCC(N(C3CCC(C4CCC5CCC6CCCCC6C5C4)CC3)C3CCC(C4CCCC5C6CCC7OC8CCCCC8C7C6OC45)CC3)CC2)CC1 ; C1CCC(C2CCC(C3CCC(N(C4CCC(C5CCCCC5)CC4)C4CCC(C5CCCC6C7CCC8OC9CCCCC9C8C7OC56)CC4)CC3)CC2)CC1 ; C1CCC(C2CCC(N(C3CCC(C4CCCCC4C4CCCCC4)CC3)C3CCC(C4CCCC5C6CCC7OC8CCCCC8C7C6OC45)CC3)CC2)CC1 ; [2H]C1C([2H])C([2H])C(C2CCC(N(C3CCC(C4CCCC5C6CCC7OC8CCCCC8C7C6OC45)CC3)C3CCC(C4C([2H])C([2H])C([2H])C([2H])C4[2H])CC3)CC2)C([2H])C1[2H]]

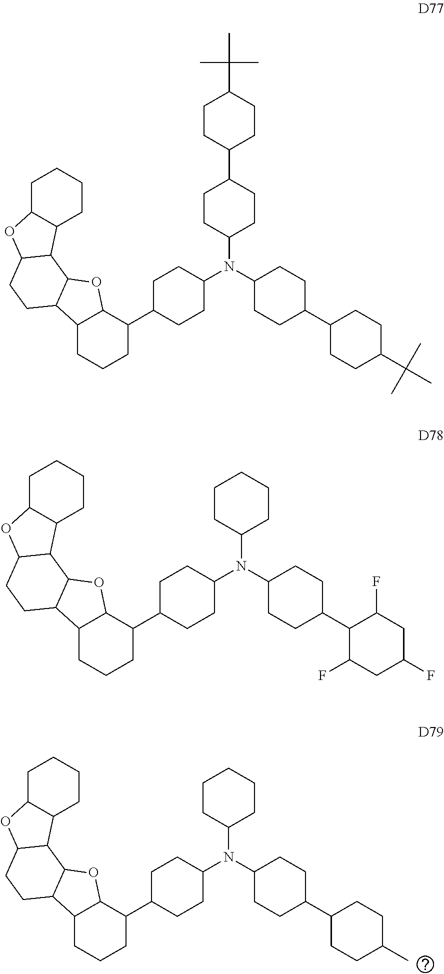 [SMILES: CC(C)(C)C1CCC(C2CCC(N(C3CCC(C4CCC(C(C)(C)C)CC4)CC3)C3CCC(C4CCCC5C6CCC7OC8CCCCC8C7C6OC45)CC3)CC2)CC1 ; FC1CC(F)C(C2CCC(N(C3CCCCC3)C3CCC(C4CCCC5C6CCC7OC8CCCCC8C7C6OC45)CC3)CC2)C(F)C1 ; CC1CCC(C2CCC(N(C3CCCCC3)C3CCC(C4CCCC5C6CCC7OC8CCCCC8C7C6OC45)CC3)CC2)CC1]

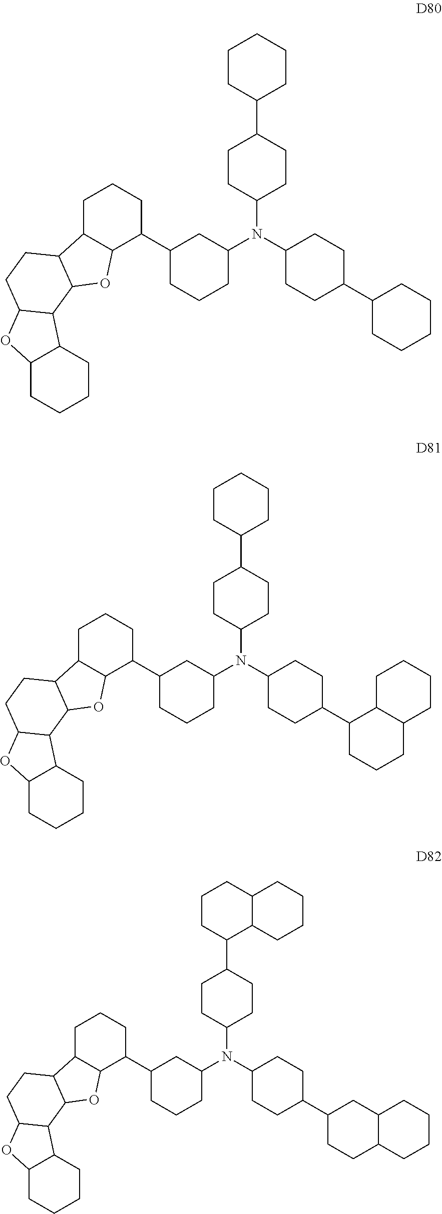 [SMILES: C1CCC(C2CCC(N(C3CCC(C4CCCCC4)CC3)C3CCCC(C4CCCC5C6CCC7OC8CCCCC8C7C6OC45)C3)CC2)CC1 ; C1CCC(C2CCC(N(C3CCC(C4CCCC5CCCCC54)CC3)C3CCCC(C4CCCC5C6CCC7OC8CCCCC8C7C6OC45)C3)CC2)CC1 ; C1CCC2CC(C3CCC(N(C4CCC(C5CCCC6CCCCC65)CC4)C4CCCC(C5CCCC6C7CCC8OC9CCCCC9C8C7OC56)C4)CC3)CCC2C1]

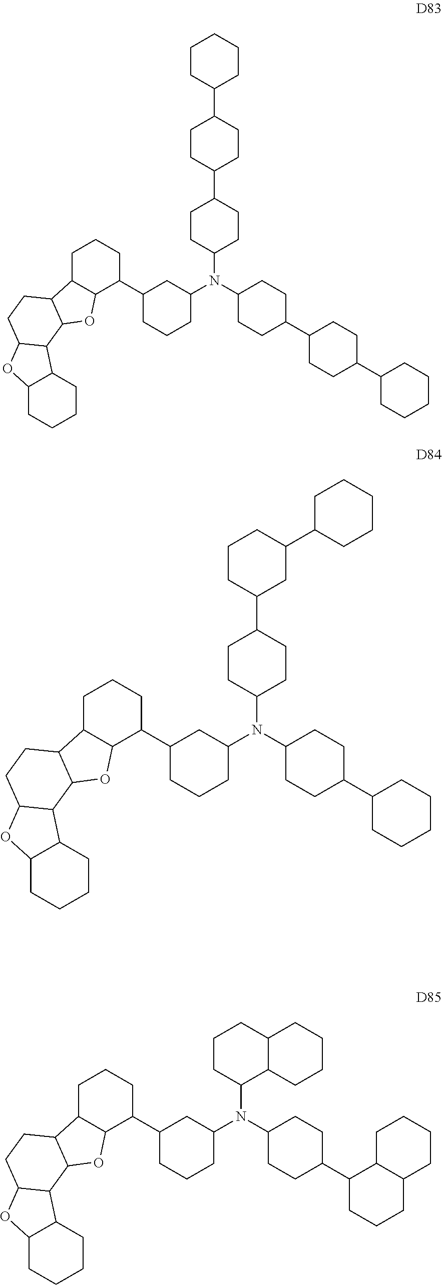 [SMILES: C1CCC(C2CCC(C3CCC(N(C4CCC(C5CCC(C6CCCCC6)CC5)CC4)C4CCCC(C5CCCC6C7CCC8OC9CCCCC9C8C7OC56)C4)CC3)CC2)CC1 ; C1CCC(C2CCC(N(C3CCC(C4CCCC(C5CCCCC5)C4)CC3)C3CCCC(C4CCCC5C6CCC7OC8CCCCC8C7C6OC45)C3)CC2)CC1 ; C1CCC2C(C1)CCCC2C1CCC(N(C2CCCC(C3CCCC4C5CCC6OC7CCCCC7C6C5OC34)C2)C2CCCC3CCCCC32)CC1]

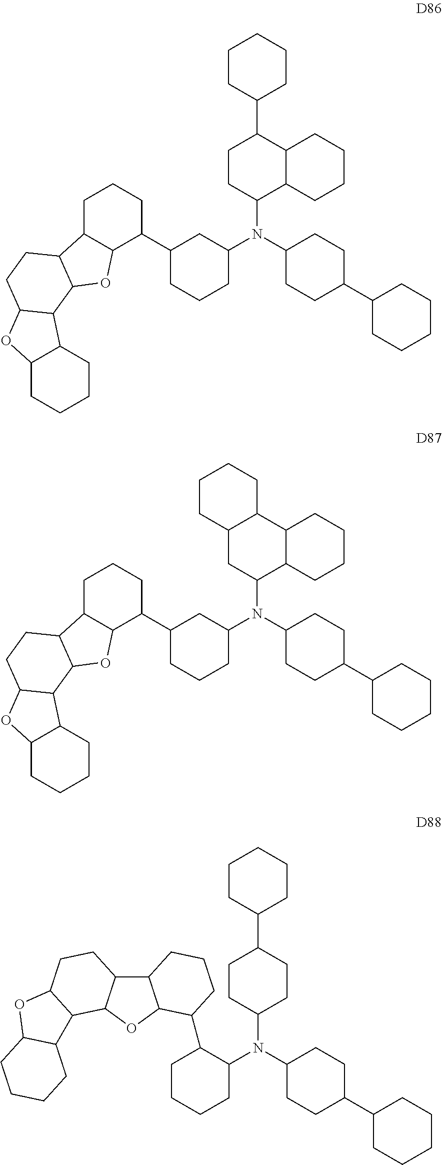 [SMILES: C1CCC(C2CCC(N(C3CCCC(C4CCCC5C6CCC7OC8CCCCC8C7C6OC45)C3)C3CCC(C4CCCCC4)C4CCCCC43)CC2)CC1 ; C1CCC(C2CCC(N(C3CCCC(C4CCCC5C6CCC7OC8CCCCC8C7C6OC45)C3)C3CC4CCCCC4C4CCCCC43)CC2)CC1 ; C1CCC(C2CCC(N(C3CCC(C4CCCCC4)CC3)C3CCCCC3C3CCCC4C5CCC6OC7CCCCC7C6C5OC43)CC2)CC1]

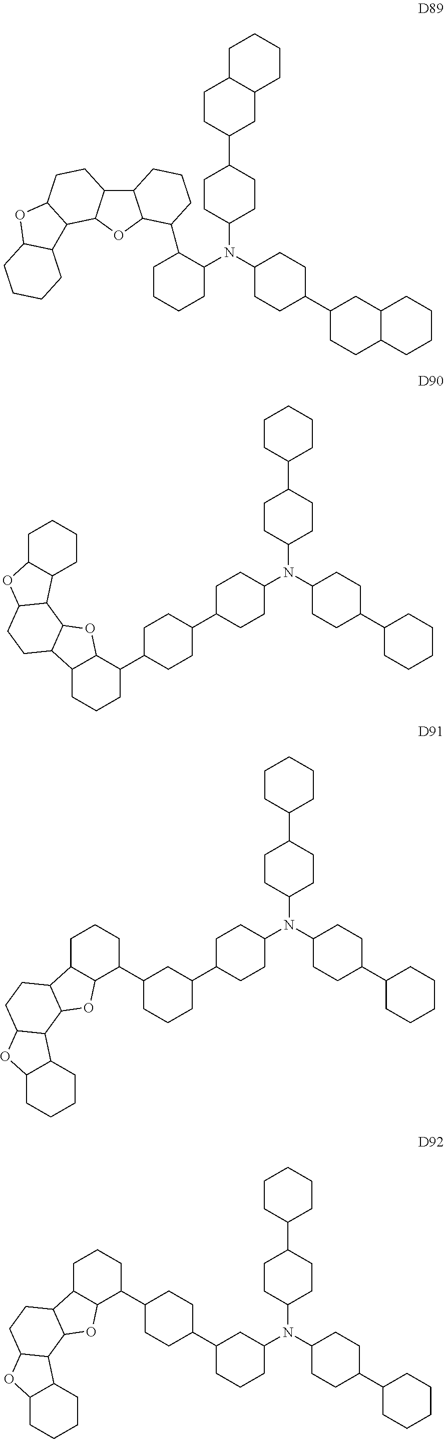 [SMILES: C1CCC2CC(C3CCC(N(C4CCC(C5CCC6CCCCC6C5)CC4)C4CCCCC4C4CCCC5C6CCC7OC8CCCCC8C7C6OC54)CC3)CCC2C1 ; C1CCC(C2CCC(N(C3CCC(C4CCCCC4)CC3)C3CCC(C4CCC(C5CCCC6C7CCC8OC9CCCCC9C8C7OC56)CC4)CC3)CC2)CC1 ; C1CCC(C2CCC(N(C3CCC(C4CCCCC4)CC3)C3CCC(C4CCCC(C5CCCC6C7CCC8OC9CCCCC9C8C7OC56)C4)CC3)CC2)CC1 ; C1CCC(C2CCC(N(C3CCC(C4CCCCC4)CC3)C3CCCC(C4CCC(C5CCCC6C7CCC8OC9CCCCC9C8C7OC56)CC4)C3)CC2)CC1]

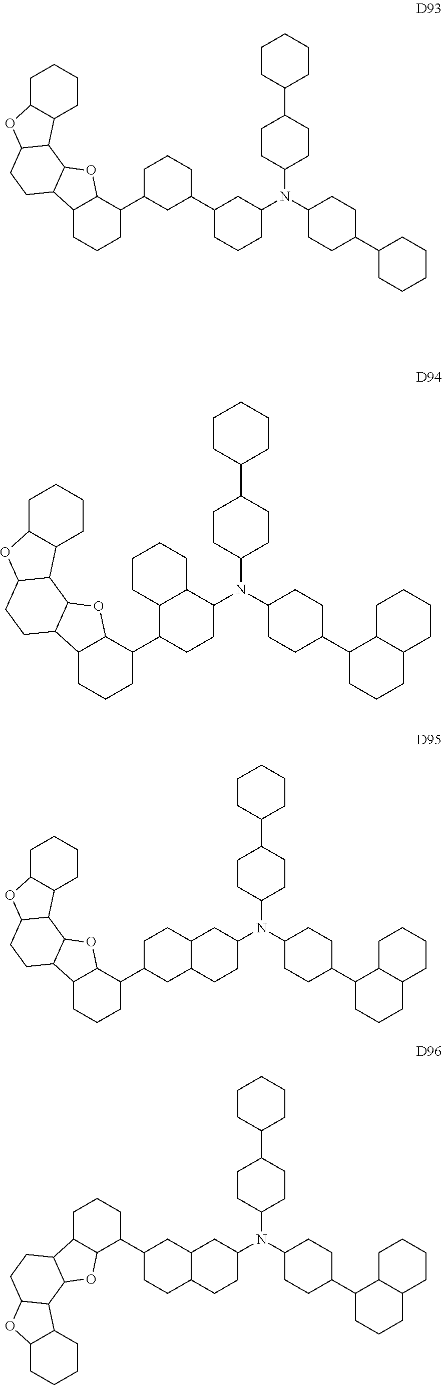 [SMILES: C1CCC(C2CCC(N(C3CCC(C4CCCCC4)CC3)C3CCCC(C4CCCC(C5CCCC6C7CCC8OC9CCCCC9C8C7OC56)C4)C3)CC2)CC1 ; C1CCC(C2CCC(N(C3CCC(C4CCCC5CCCCC54)CC3)C3CCC(C4CCCC5C6CCC7OC8CCCCC8C7C6OC45)C4CCCCC43)CC2)CC1 ; C1CCC(C2CCC(N(C3CCC(C4CCCC5CCCCC54)CC3)C3CCC4CC(C5CCCC6C7CCC8OC9CCCCC9C8C7OC56)CCC4C3)CC2)CC1 ; C1CCC(C2CCC(N(C3CCC(C4CCCC5CCCCC54)CC3)C3CCC4CCC(C5CCCC6C7CCC8OC9CCCCC9C8C7OC56)CC4C3)CC2)CC1]

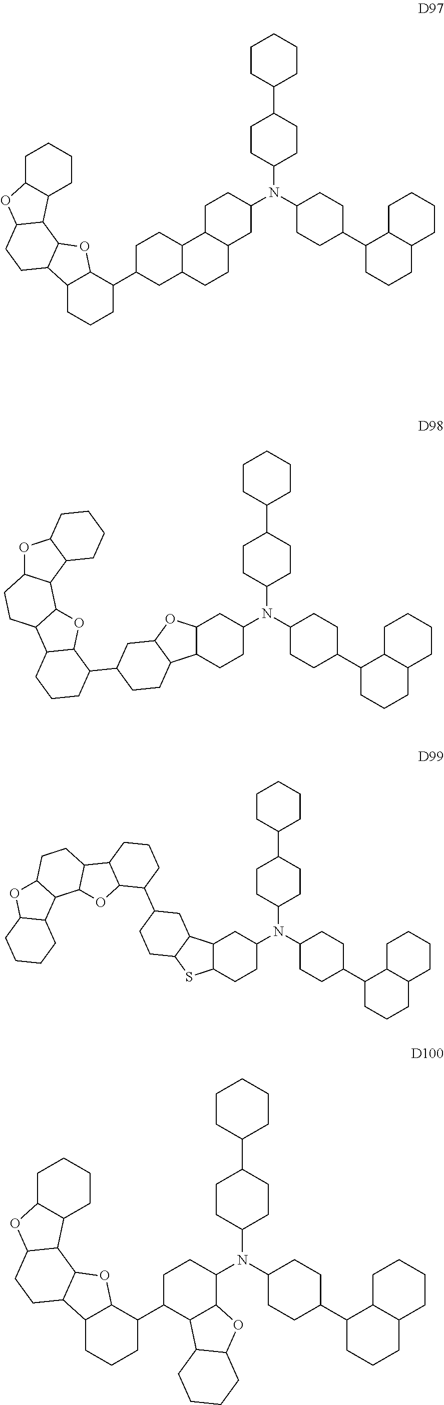 [SMILES: C1CCC(C2CCC(N(C3CCC(C4CCCC5CCCCC54)CC3)C3CCC4C(CCC5CC(C6CCCC7C8CCC9OC%10CCCCC%10C9C8OC67)CCC54)C3)CC2)CC1 ; C1CCC(C2CCC(N(C3CCC(C4CCCC5CCCCC54)CC3)C3CCC4C(C3)OC3CC(C5CCCC6C7CCC8OC9CCCCC9C8C7OC56)CCC34)CC2)CC1 ; C1CCC(C2CCC(N(C3CCC(C4CCCC5CCCCC54)CC3)C3CCC4SC5CCC(C6CCCC7C8CCC9OC%10CCCCC%10C9C8OC67)CC5C4C3)CC2)CC1 ; C1CCC(C2CCC(N(C3CCC(C4CCCC5CCCCC54)CC3)C3CCC(C4CCCC5C6CCC7OC8CCCCC8C7C6OC54)C4C5CCCCC5OC43)CC2)CC1]

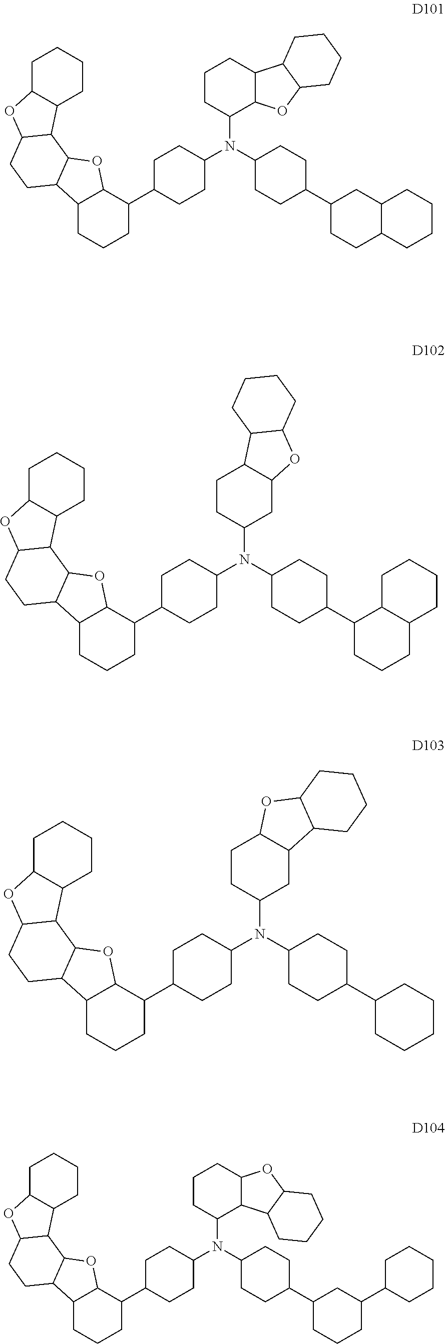 [SMILES: C1CCC2CC(C3CCC(N(C4CCC(C5CCCC6C7CCC8OC9CCCCC9C8C7OC56)CC4)C4CCCC5C6CCCCC6OC54)CC3)CCC2C1 ; C1CCC2C(C1)CCCC2C1CCC(N(C2CCC(C3CCCC4C5CCC6OC7CCCCC7C6C5OC34)CC2)C2CCC3C(C2)OC2CCCCC23)CC1 ; C1CCC(C2CCC(N(C3CCC(C4CCCC5C6CCC7OC8CCCCC8C7C6OC45)CC3)C3CCC4OC5CCCCC5C4C3)CC2)CC1 ; C1CCC(C2CCCC(C3CCC(N(C4CCC(C5CCCC6C7CCC8OC9CCCCC9C8C7OC56)CC4)C4CCCC5OC6CCCCC6C54)CC3)C2)CC1]

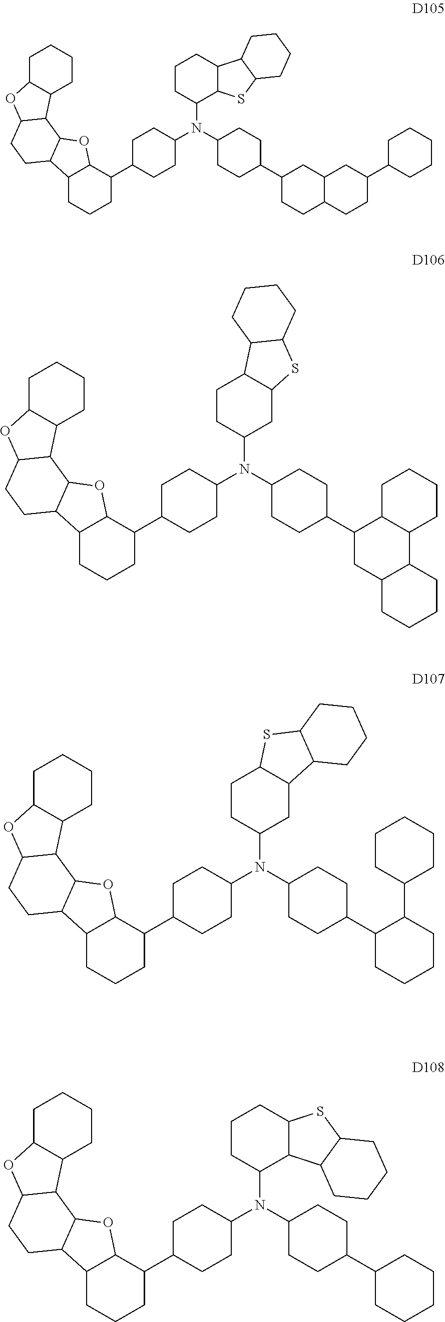 [SMILES: C1CCC(C2CCC3CCC(C4CCC(N(C5CCC(C6CCCC7C8CCC9OC%10CCCCC%10C9C8OC67)CC5)C5CCCC6C7CCCCC7SC65)CC4)CC3C2)CC1 ; C1CCC2C(C1)CC(C1CCC(N(C3CCC(C4CCCC5C6CCC7OC8CCCCC8C7C6OC45)CC3)C3CCC4C(C3)SC3CCCCC34)CC1)C1CCCCC21 ; C1CCC(C2CCCCC2C2CCC(N(C3CCC(C4CCCC5C6CCC7OC8CCCCC8C7C6OC45)CC3)C3CCC4SC5CCCCC5C4C3)CC2)CC1 ; C1CCC(C2CCC(N(C3CCC(C4CCCC5C6CCC7OC8CCCCC8C7C6OC45)CC3)C3CCCC4SC5CCCCC5C43)CC2)CC1]

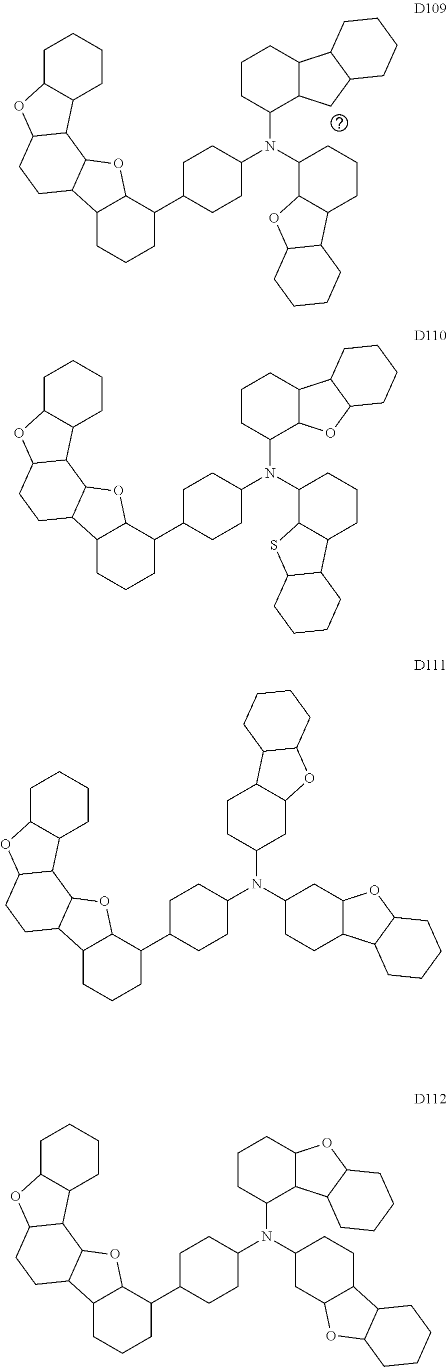 [SMILES: C1CCC2C(C1)CC1C2CCCC1N(C1CCC(C2CCCC3C4CCC5OC6CCCCC6C5C4OC23)CC1)C1CCCC2C3CCCCC3OC21 ; C1CCC2C(C1)OC1C2CCCC1N(C1CCC(C2CCCC3C4CCC5OC6CCCCC6C5C4OC23)CC1)C1CCCC2C3CCCCC3SC21 ; C1CCC2C(C1)OC1CC(N(C3CCC(C4CCCC5C6CCC7OC8CCCCC8C7C6OC45)CC3)C3CCC4C(C3)OC3CCCCC34)CCC12 ; C1CCC2C(C1)OC1CC(N(C3CCC(C4CCCC5C6CCC7OC8CCCCC8C7C6OC45)CC3)C3CCCC4OC5CCCCC5C43)CCC12]

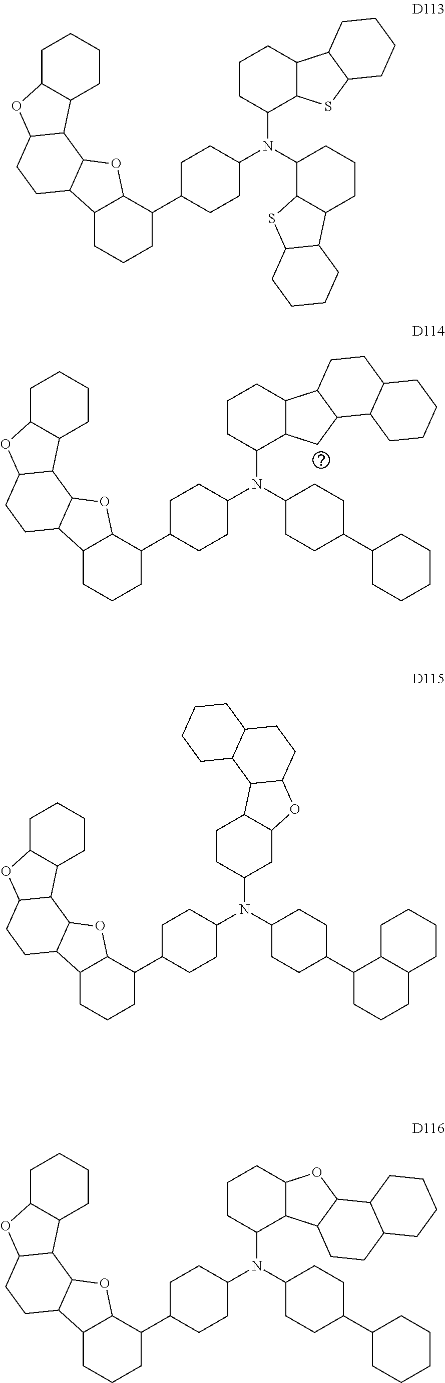 [SMILES: C1CCC2C(C1)OC1CCC3C4CCCC(C5CCC(N(C6CCCC7C8CCCCC8SC76)C6CCCC7C8CCCCC8SC76)CC5)C4OC3C12 ; C1CCC(C2CCC(N(C3CCC(C4CCCC5C6CCC7OC8CCCCC8C7C6OC45)CC3)C3CCCC4C5CCC6CCCCC6C5CC43)CC2)CC1 ; C1CCC2C(C1)CCCC2C1CCC(N(C2CCC(C3CCCC4C5CCC6OC7CCCCC7C6C5OC34)CC2)C2CCC3C(C2)OC2CCC4CCCCC4C23)CC1 ; C1CCC(C2CCC(N(C3CCC(C4CCCC5C6CCC7OC8CCCCC8C7C6OC45)CC3)C3CCCC4OC5C6CCCCC6CCC5C43)CC2)CC1]

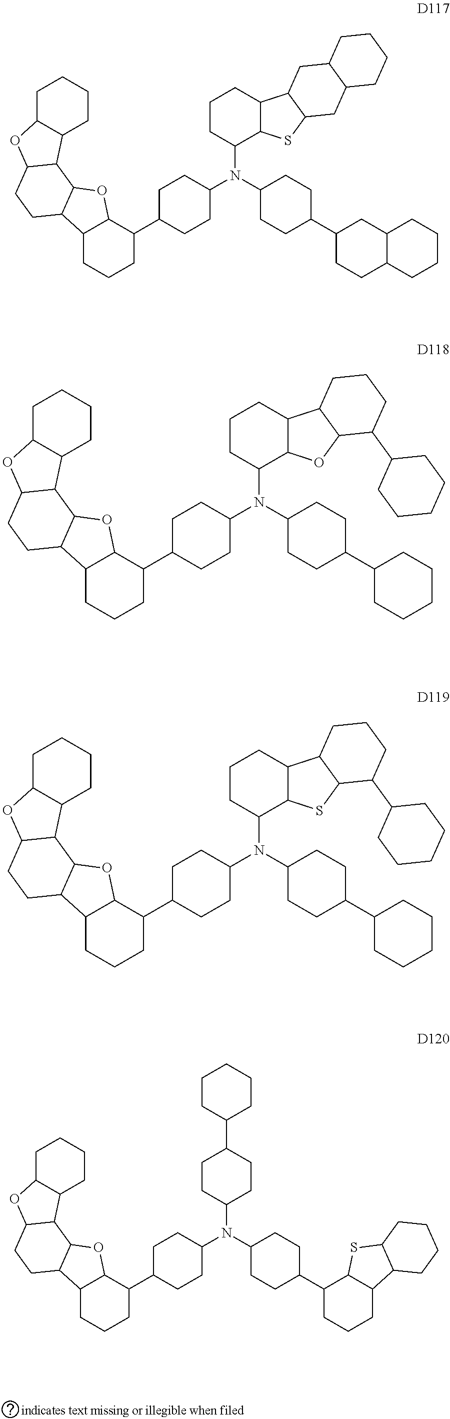 [SMILES: C1CCC2CC(C3CCC(N(C4CCC(C5CCCC6C7CCC8OC9CCCCC9C8C7OC56)CC4)C4CCCC5C6CC7CCCCC7CC6SC54)CC3)CCC2C1 ; C1CCC(C2CCC(N(C3CCC(C4CCCC5C6CCC7OC8CCCCC8C7C6OC45)CC3)C3CCCC4C5CCCC(C6CCCCC6)C5OC43)CC2)CC1 ; C1CCC(C2CCC(N(C3CCC(C4CCCC5C6CCC7OC8CCCCC8C7C6OC45)CC3)C3CCCC4C5CCCC(C6CCCCC6)C5SC43)CC2)CC1 ; C1CCC(C2CCC(N(C3CCC(C4CCCC5C6CCC7OC8CCCCC8C7C6OC45)CC3)C3CCC(C4CCCC5C6CCCCC6SC45)CC3)CC2)CC1]

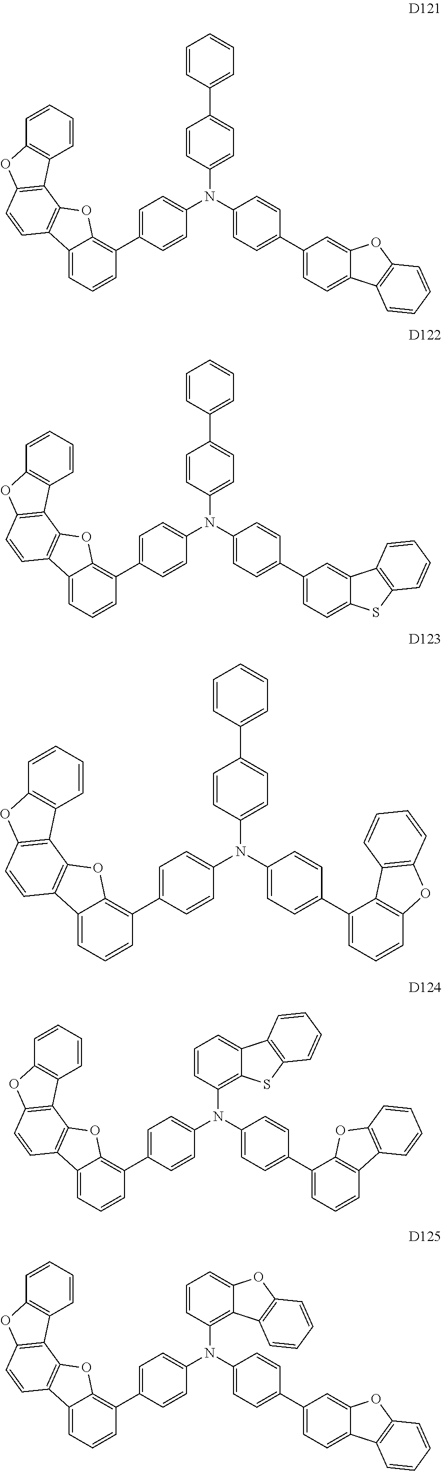 [SMILES: c1ccc(-c2ccc(N(c3ccc(-c4ccc5c(c4)oc4ccccc45)cc3)c3ccc(-c4cccc5c4oc4c5ccc5oc6ccccc6c54)cc3)cc2)cc1 ; c1ccc(-c2ccc(N(c3ccc(-c4ccc5sc6ccccc6c5c4)cc3)c3ccc(-c4cccc5c4oc4c5ccc5oc6ccccc6c54)cc3)cc2)cc1 ; c1ccc(-c2ccc(N(c3ccc(-c4cccc5c4oc4c5ccc5oc6ccccc6c54)cc3)c3ccc(-c4cccc5oc6ccccc6c45)cc3)cc2)cc1 ; c1ccc2c(c1)oc1c(-c3ccc(N(c4ccc(-c5cccc6c5oc5c6ccc6oc7ccccc7c65)cc4)c4cccc5c4sc4ccccc45)cc3)cccc12 ; c1ccc2c(c1)oc1cc(-c3ccc(N(c4ccc(-c5cccc6c5oc5c6ccc6oc7ccccc7c65)cc4)c4cccc5oc6ccccc6c45)cc3)ccc12]

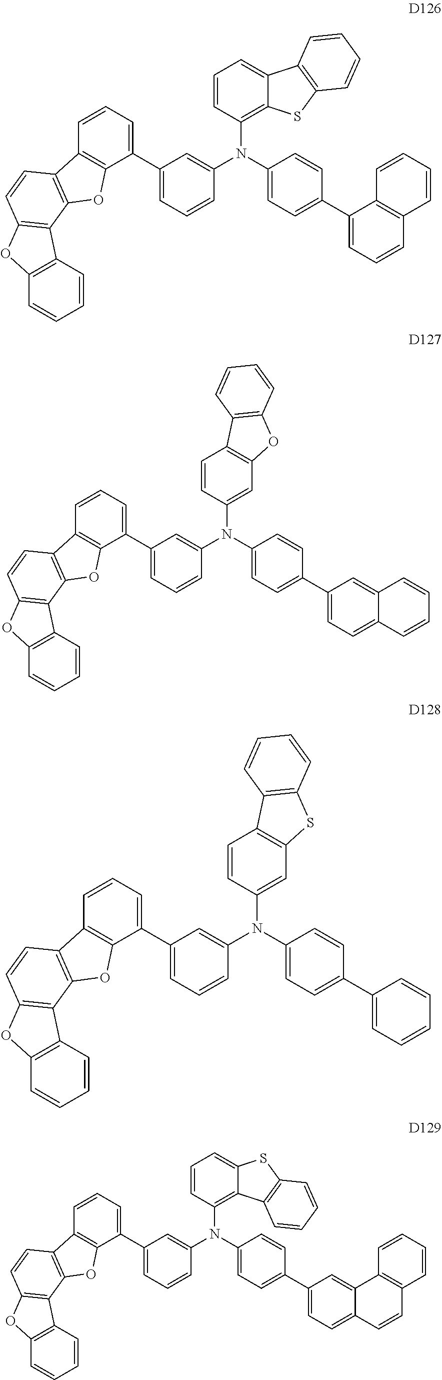 [SMILES: c1cc(-c2cccc3c2oc2c3ccc3oc4ccccc4c32)cc(N(c2ccc(-c3cccc4ccccc34)cc2)c2cccc3c2sc2ccccc23)c1 ; c1cc(-c2cccc3c2oc2c3ccc3oc4ccccc4c32)cc(N(c2ccc(-c3ccc4ccccc4c3)cc2)c2ccc3c(c2)oc2ccccc23)c1 ; c1ccc(-c2ccc(N(c3cccc(-c4cccc5c4oc4c5ccc5oc6ccccc6c54)c3)c3ccc4c(c3)sc3ccccc34)cc2)cc1 ; c1cc(-c2cccc3c2oc2c3ccc3oc4ccccc4c32)cc(N(c2ccc(-c3ccc4ccc5ccccc5c4c3)cc2)c2cccc3sc4ccccc4c23)c1]

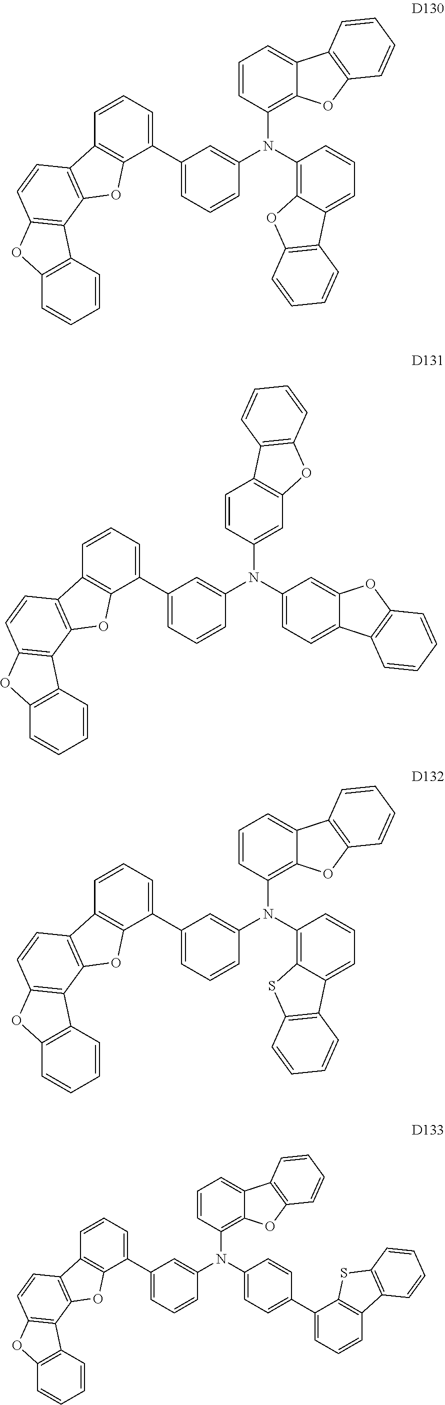 [SMILES: c1cc(-c2cccc3c2oc2c3ccc3oc4ccccc4c32)cc(N(c2cccc3c2oc2ccccc23)c2cccc3c2oc2ccccc23)c1 ; c1cc(-c2cccc3c2oc2c3ccc3oc4ccccc4c32)cc(N(c2ccc3c(c2)oc2ccccc23)c2ccc3c(c2)oc2ccccc23)c1 ; c1cc(-c2cccc3c2oc2c3ccc3oc4ccccc4c32)cc(N(c2cccc3c2oc2ccccc23)c2cccc3c2sc2ccccc23)c1 ; c1cc(-c2cccc3c2oc2c3ccc3oc4ccccc4c32)cc(N(c2ccc(-c3cccc4c3sc3ccccc34)cc2)c2cccc3c2oc2ccccc23)c1]

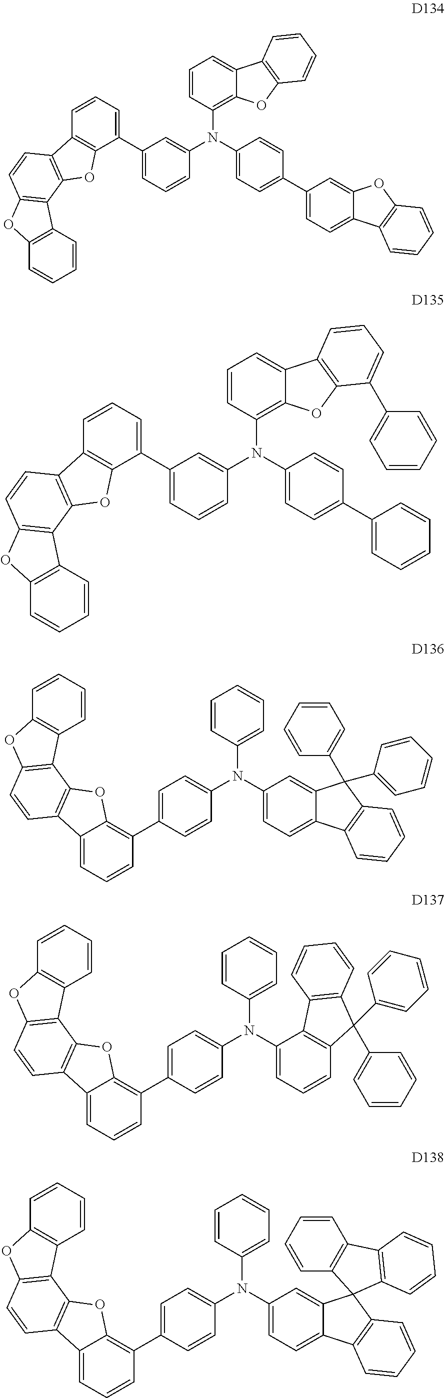

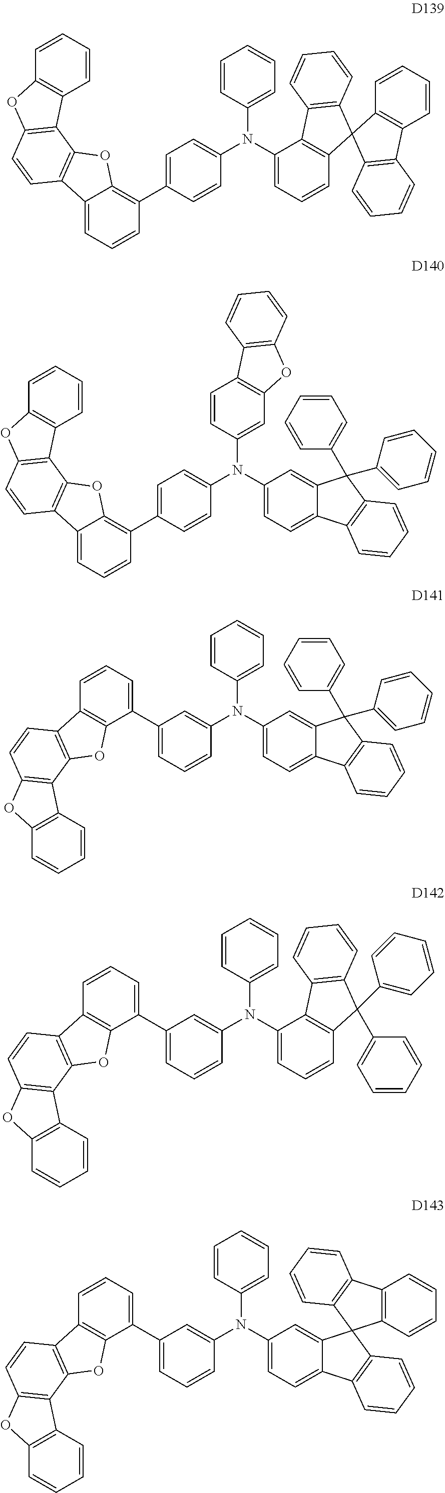 [SMILES: c1ccc(N(c2ccc(-c3cccc4c3oc3c4ccc4oc5ccccc5c43)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6c5oc5c6ccc6oc7ccccc7c65)cc4)c4ccc5c(c4)oc4ccccc45)cc32)cc1 ; c1ccc(N(c2cccc(-c3cccc4c3oc3c4ccc4oc5ccccc5c43)c2)c2ccc3c(c2)C(c2ccccc2)(c2ccccc2)c2ccccc2-3)cc1 ; c1ccc(N(c2cccc(-c3cccc4c3oc3c4ccc4oc5ccccc5c43)c2)c2cccc3c2-c2ccccc2C3(c2ccccc2)c2ccccc2)cc1 ; c1ccc(N(c2cccc(-c3cccc4c3oc3c4ccc4oc5ccccc5c43)c2)c2ccc3c(c2)C2(c4ccccc4-c4ccccc42)c2ccccc2-3)cc1]

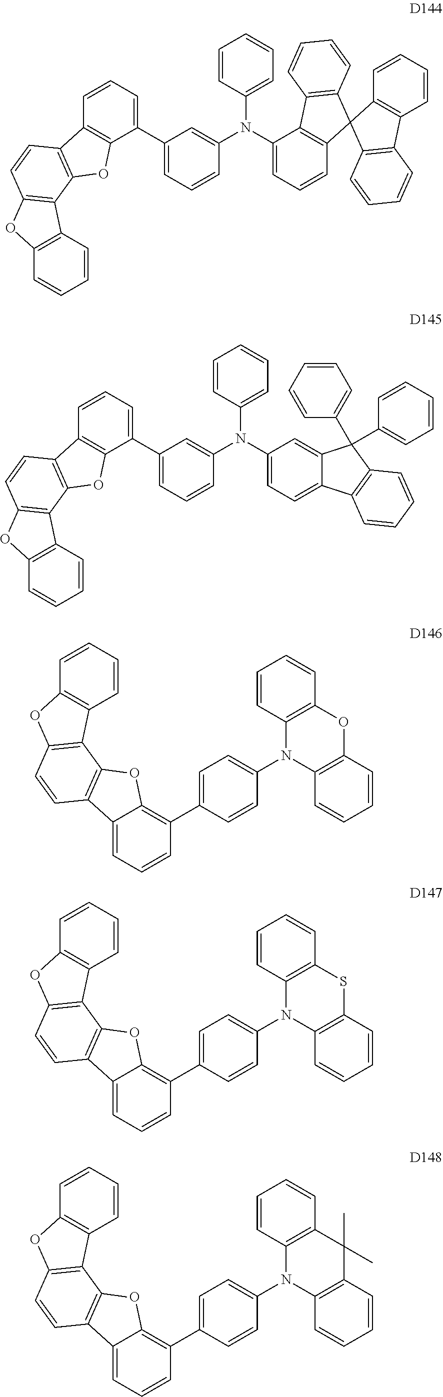 [SMILES: c1ccc(N(c2cccc(-c3cccc4c3oc3c4ccc4oc5ccccc5c43)c2)c2cccc3c2-c2ccccc2C32c3ccccc3-c3ccccc32)cc1 ; c1ccc(N(c2cccc(-c3cccc4c3oc3c4ccc4oc5ccccc5c43)c2)c2ccc3c(c2)C(c2ccccc2)(c2ccccc2)c2ccccc2-3)cc1 ; c1ccc2c(c1)Oc1ccccc1N2c1ccc(-c2cccc3c2oc2c3ccc3oc4ccccc4c32)cc1 ; c1ccc2c(c1)Sc1ccccc1N2c1ccc(-c2cccc3c2oc2c3ccc3oc4ccccc4c32)cc1 ; CC1(C)c2ccccc2N(c2ccc(-c3cccc4c3oc3c4ccc4oc5ccccc5c43)cc2)c2ccccc21]

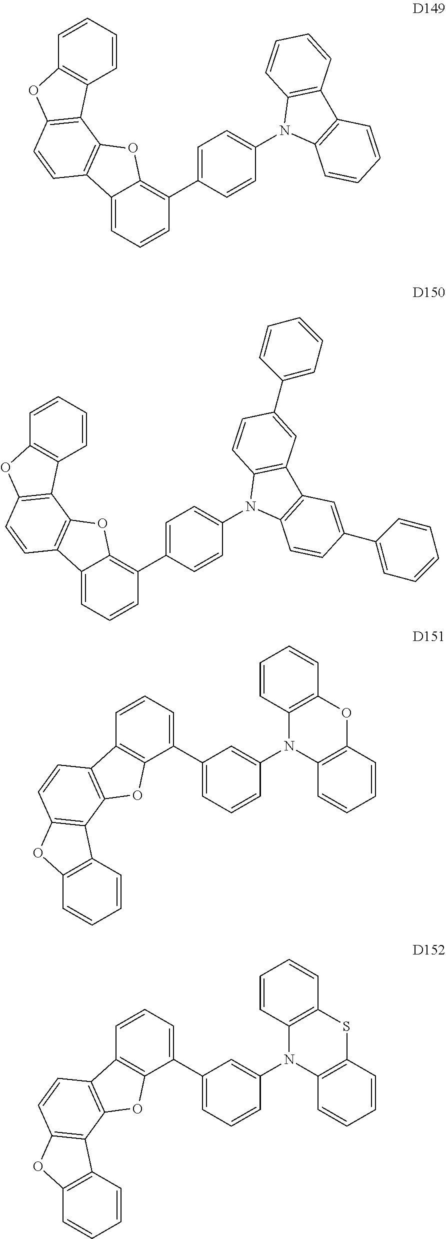 [SMILES: c1ccc2c(c1)oc1ccc3c4cccc(-c5ccc(-n6c7ccccc7c7ccccc76)cc5)c4oc3c12 ; c1ccc(-c2ccc3c(c2)c2cc(-c4ccccc4)ccc2n3-c2ccc(-c3cccc4c3oc3c4ccc4oc5ccccc5c43)cc2)cc1 ; c1cc(-c2cccc3c2oc2c3ccc3oc4ccccc4c32)cc(N2c3ccccc3Oc3ccccc32)c1 ; c1cc(-c2cccc3c2oc2c3ccc3oc4ccccc4c32)cc(N2c3ccccc3Sc3ccccc32)c1]

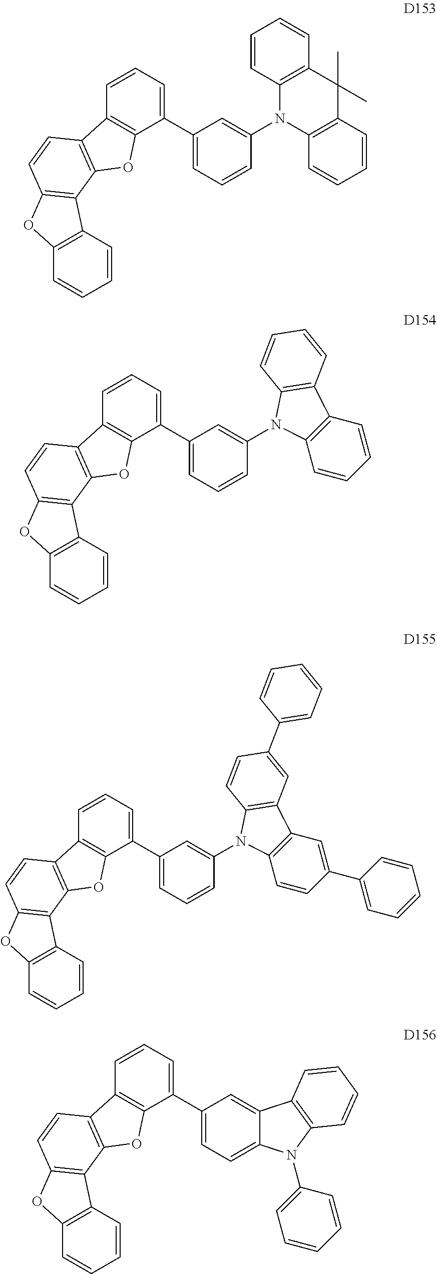 [SMILES: CC1(C)c2ccccc2N(c2cccc(-c3cccc4c3oc3c4ccc4oc5ccccc5c43)c2)c2ccccc21 ; c1cc(-c2cccc3c2oc2c3ccc3oc4ccccc4c32)cc(-n2c3ccccc3c3ccccc32)c1 ; c1ccc(-c2ccc3c(c2)c2cc(-c4ccccc4)ccc2n3-c2cccc(-c3cccc4c3oc3c4ccc4oc5ccccc5c43)c2)cc1 ; c1ccc(-n2c3ccccc3c3cc(-c4cccc5c4oc4c5ccc5oc6ccccc6c54)ccc32)cc1]

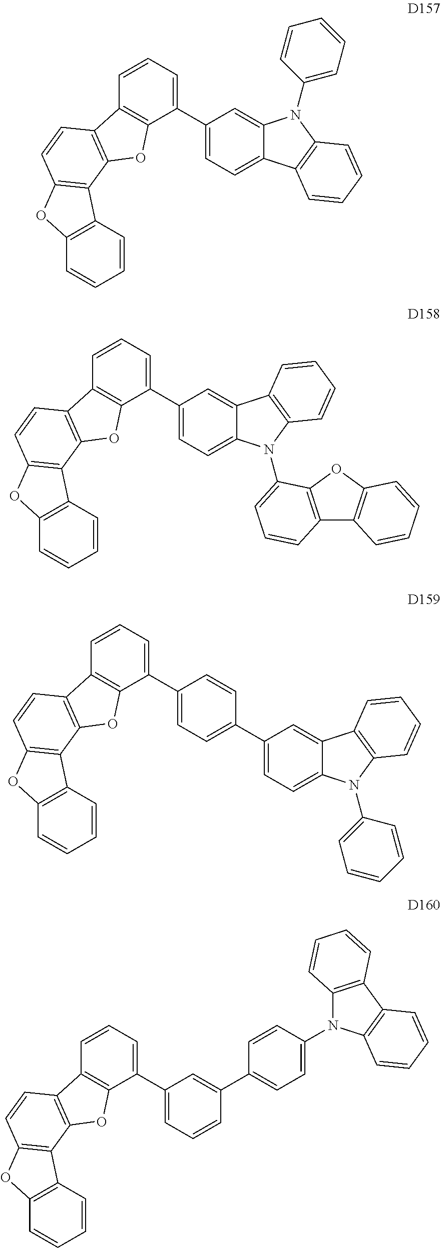 [SMILES: c1ccc(-n2c3ccccc3c3ccc(-c4cccc5c4oc4c5ccc5oc6ccccc6c54)cc32)cc1 ; c1ccc2c(c1)oc1c(-n3c4ccccc4c4cc(-c5cccc6c5oc5c6ccc6oc7ccccc7c65)ccc43)cccc12 ; c1ccc(-n2c3ccccc3c3cc(-c4ccc(-c5cccc6c5oc5c6ccc6oc7ccccc7c65)cc4)ccc32)cc1 ; c1cc(-c2ccc(-n3c4ccccc4c4ccccc43)cc2)cc(-c2cccc3c2oc2c3ccc3oc4ccccc4c32)c1]

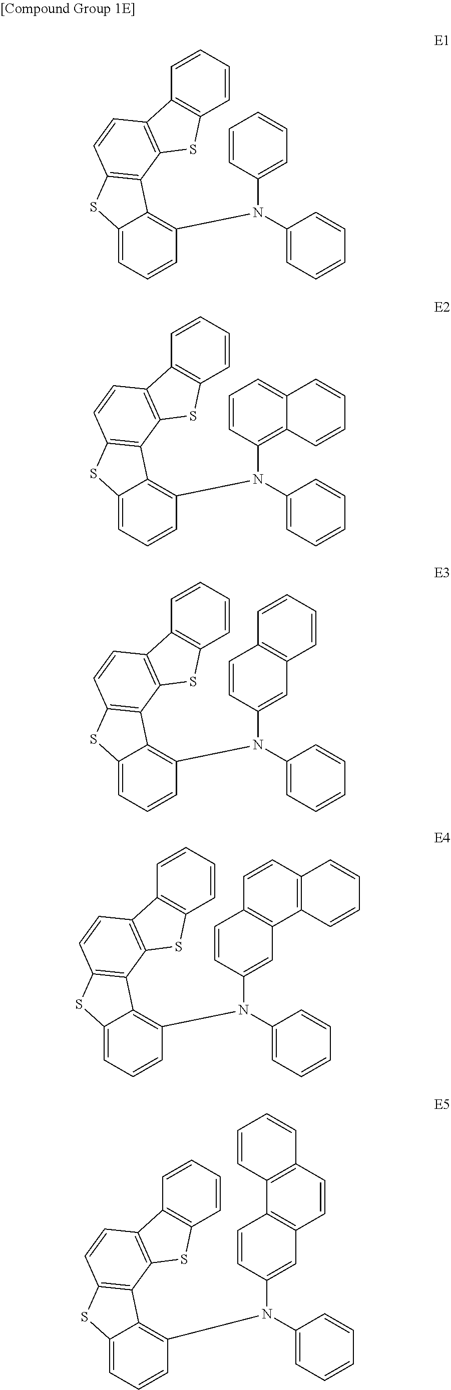 [SMILES: c1ccc(N(c2ccccc2)c2cccc3sc4ccc5c6ccccc6sc5c4c23)cc1 ; c1ccc(N(c2cccc3ccccc23)c2cccc3sc4ccc5c6ccccc6sc5c4c23)cc1 ; c1ccc(N(c2ccc3ccccc3c2)c2cccc3sc4ccc5c6ccccc6sc5c4c23)cc1 ; c1ccc(N(c2ccc3ccc4ccccc4c3c2)c2cccc3sc4ccc5c6ccccc6sc5c4c23)cc1 ; c1ccc(N(c2ccc3c(ccc4ccccc43)c2)c2cccc3sc4ccc5c6ccccc6sc5c4c23)cc1]

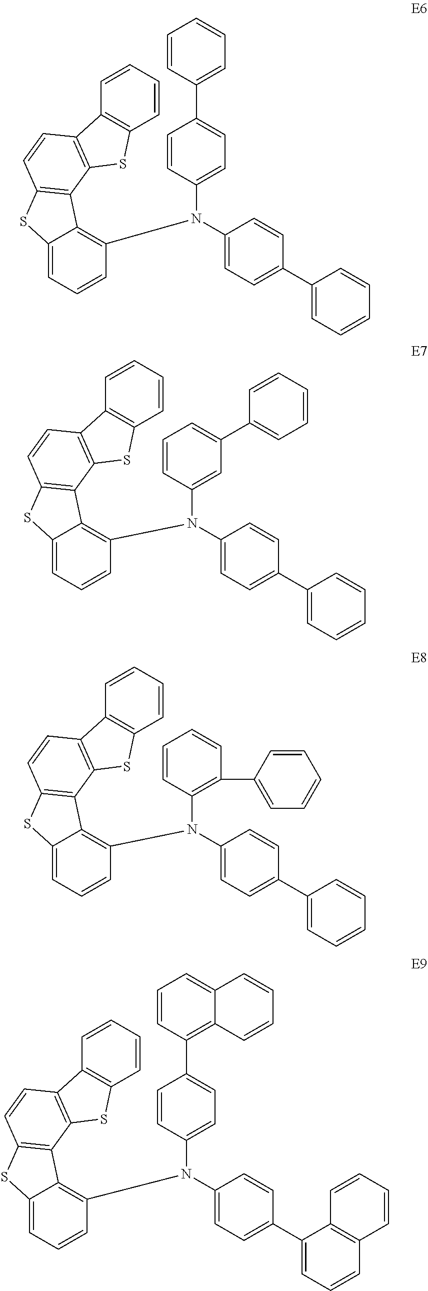 [SMILES: c1ccc(-c2ccc(N(c3ccc(-c4ccccc4)cc3)c3cccc4sc5ccc6c7ccccc7sc6c5c34)cc2)cc1 ; c1ccc(-c2ccc(N(c3cccc(-c4ccccc4)c3)c3cccc4sc5ccc6c7ccccc7sc6c5c34)cc2)cc1 ; c1ccc(-c2ccc(N(c3ccccc3-c3ccccc3)c3cccc4sc5ccc6c7ccccc7sc6c5c34)cc2)cc1 ; c1ccc2c(-c3ccc(N(c4ccc(-c5cccc6ccccc56)cc4)c4cccc5sc6ccc7c8ccccc8sc7c6c45)cc3)cccc2c1]

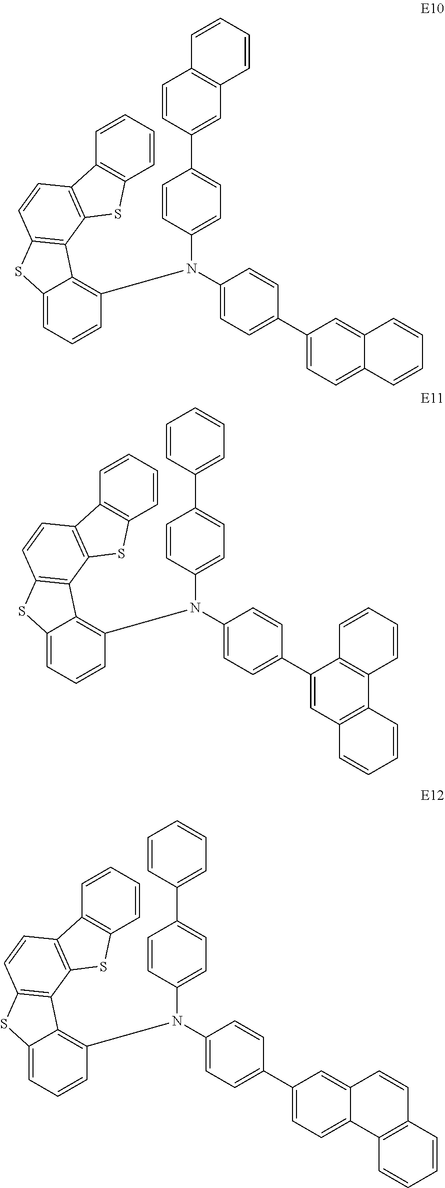 [SMILES: c1ccc2cc(-c3ccc(N(c4ccc(-c5ccc6ccccc6c5)cc4)c4cccc5sc6ccc7c8ccccc8sc7c6c45)cc3)ccc2c1 ; c1ccc(-c2ccc(N(c3ccc(-c4cc5ccccc5c5ccccc45)cc3)c3cccc4sc5ccc6c7ccccc7sc6c5c34)cc2)cc1 ; c1ccc(-c2ccc(N(c3ccc(-c4ccc5c(ccc6ccccc65)c4)cc3)c3cccc4sc5ccc6c7ccccc7sc6c5c34)cc2)cc1]

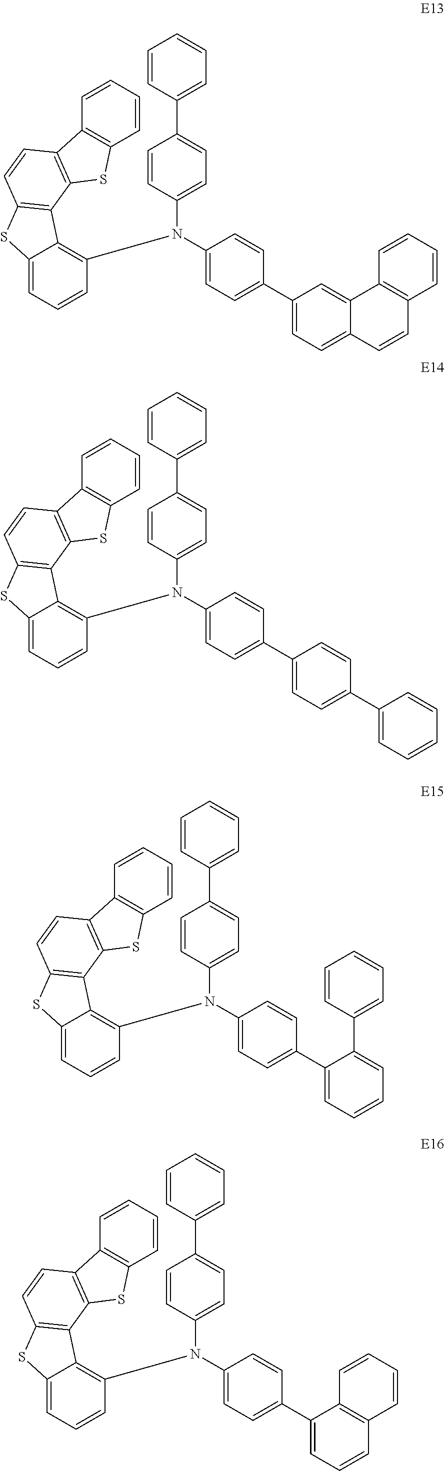 [SMILES: c1ccc(-c2ccc(N(c3ccc(-c4ccc5ccc6ccccc6c5c4)cc3)c3cccc4sc5ccc6c7ccccc7sc6c5c34)cc2)cc1 ; c1ccc(-c2ccc(-c3ccc(N(c4ccc(-c5ccccc5)cc4)c4cccc5sc6ccc7c8ccccc8sc7c6c45)cc3)cc2)cc1 ; c1ccc(-c2ccc(N(c3ccc(-c4ccccc4-c4ccccc4)cc3)c3cccc4sc5ccc6c7ccccc7sc6c5c34)cc2)cc1 ; c1ccc(-c2ccc(N(c3ccc(-c4cccc5ccccc45)cc3)c3cccc4sc5ccc6c7ccccc7sc6c5c34)cc2)cc1]

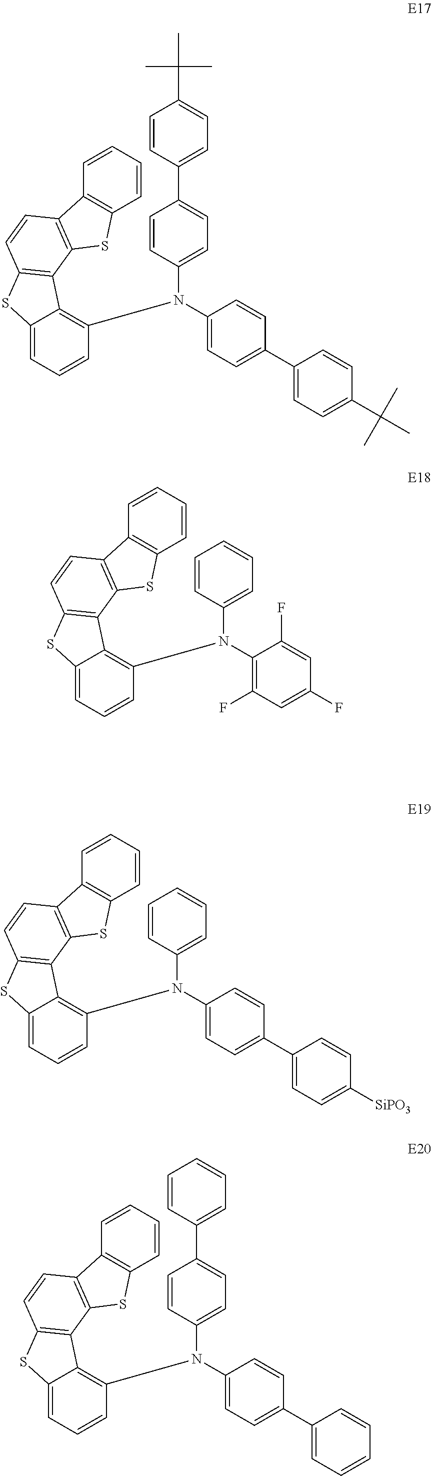

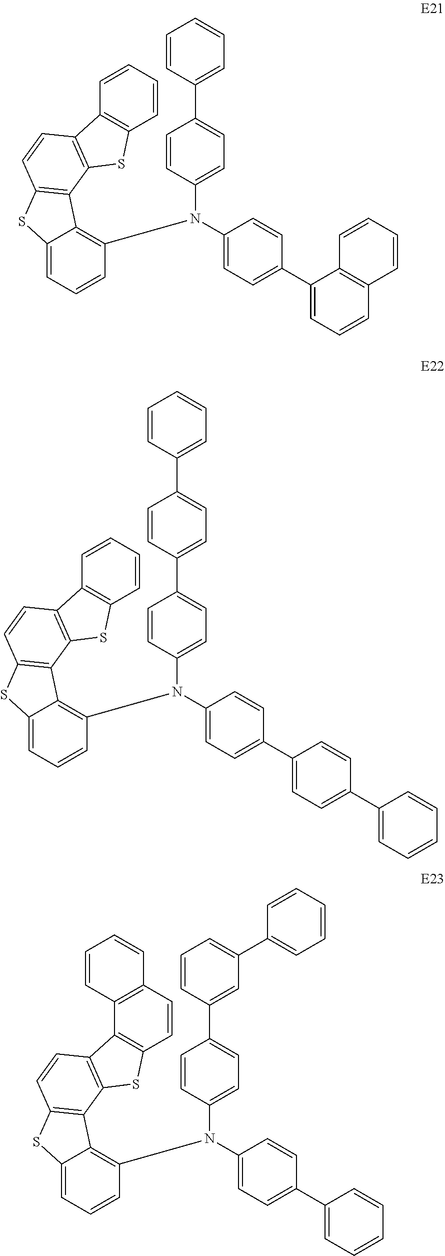 [SMILES: c1ccc(-c2ccc(N(c3ccc(-c4cccc5ccccc45)cc3)c3cccc4sc5ccc6c7ccccc7sc6c5c34)cc2)cc1 ; c1ccc(-c2ccc(-c3ccc(N(c4ccc(-c5ccc(-c6ccccc6)cc5)cc4)c4cccc5sc6ccc7c8ccccc8sc7c6c45)cc3)cc2)cc1 ; c1ccc(-c2ccc(N(c3ccc(-c4cccc(-c5ccccc5)c4)cc3)c3cccc4sc5ccc6c(sc7ccc8ccccc8c76)c5c34)cc2)cc1]

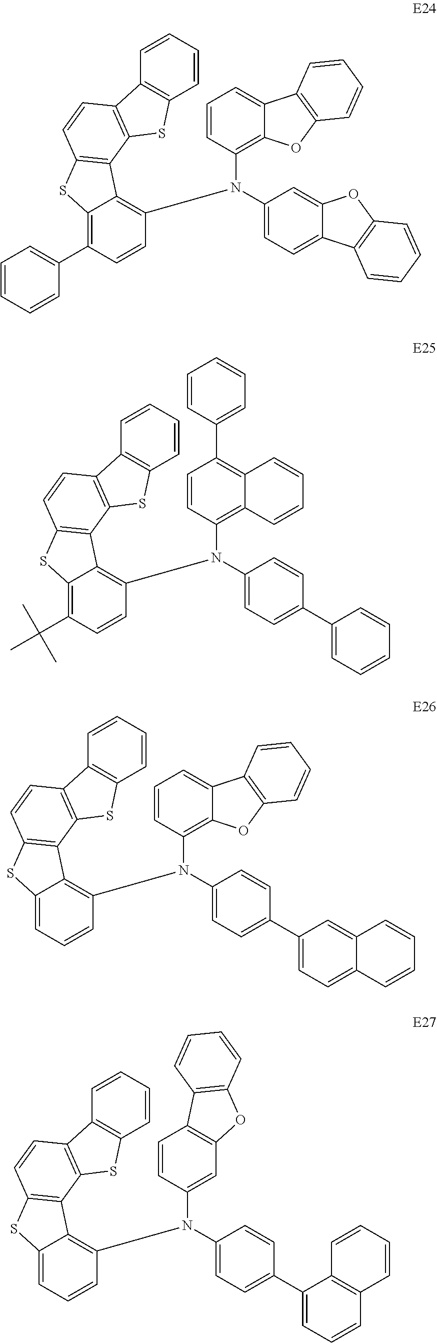 [SMILES: c1ccc(-c2ccc(N(c3ccc4c(c3)oc3ccccc34)c3cccc4c3oc3ccccc34)c3c2sc2ccc4c5ccccc5sc4c23)cc1 ; CC(C)(C)c1ccc(N(c2ccc(-c3ccccc3)cc2)c2ccc(-c3ccccc3)c3ccccc23)c2c1sc1ccc3c4ccccc4sc3c12 ; c1ccc2cc(-c3ccc(N(c4cccc5c4oc4ccccc45)c4cccc5sc6ccc7c8ccccc8sc7c6c45)cc3)ccc2c1 ; c1ccc2c(-c3ccc(N(c4ccc5c(c4)oc4ccccc45)c4cccc5sc6ccc7c8ccccc8sc7c6c45)cc3)cccc2c1]

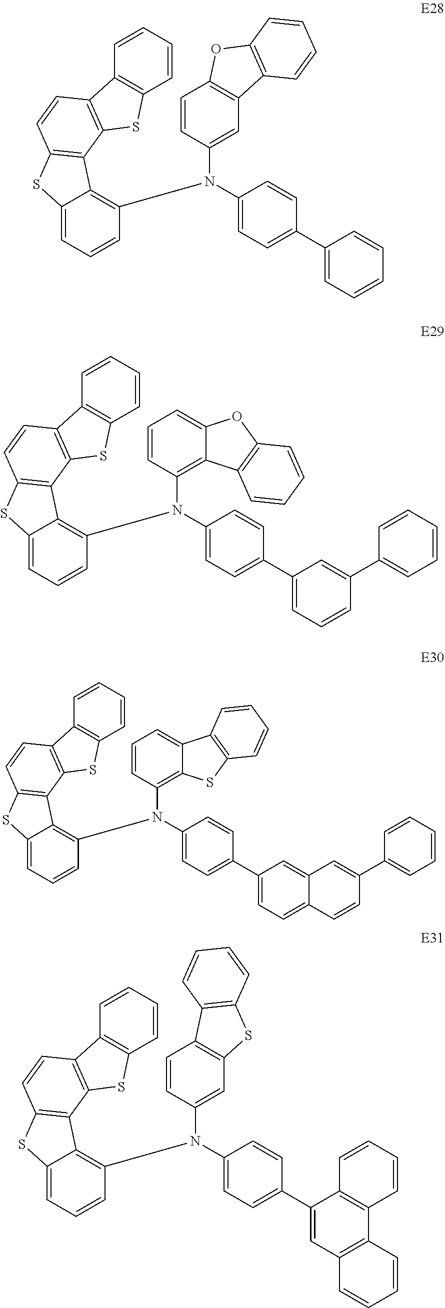 [SMILES: c1ccc(-c2ccc(N(c3ccc4oc5ccccc5c4c3)c3cccc4sc5ccc6c7ccccc7sc6c5c34)cc2)cc1 ; c1ccc(-c2cccc(-c3ccc(N(c4cccc5oc6ccccc6c45)c4cccc5sc6ccc7c8ccccc8sc7c6c45)cc3)c2)cc1 ; c1ccc(-c2ccc3ccc(-c4ccc(N(c5cccc6c5sc5ccccc56)c5cccc6sc7ccc8c9ccccc9sc8c7c56)cc4)cc3c2)cc1 ; c1ccc2c(c1)cc(-c1ccc(N(c3ccc4c(c3)sc3ccccc34)c3cccc4sc5ccc6c7ccccc7sc6c5c34)cc1)c1ccccc12]

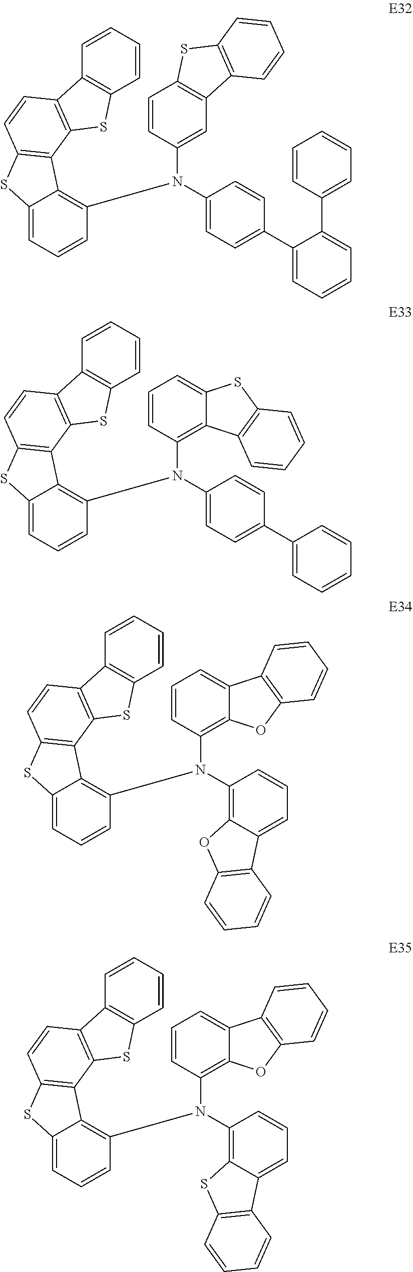 [SMILES: c1ccc(-c2ccccc2-c2ccc(N(c3ccc4sc5ccccc5c4c3)c3cccc4sc5ccc6c7ccccc7sc6c5c34)cc2)cc1 ; c1ccc(-c2ccc(N(c3cccc4sc5ccccc5c34)c3cccc4sc5ccc6c7ccccc7sc6c5c34)cc2)cc1 ; c1ccc2c(c1)oc1c(N(c3cccc4c3oc3ccccc34)c3cccc4sc5ccc6c7ccccc7sc6c5c34)cccc12 ; c1ccc2c(c1)oc1c(N(c3cccc4c3sc3ccccc34)c3cccc4sc5ccc6c7ccccc7sc6c5c34)cccc12]

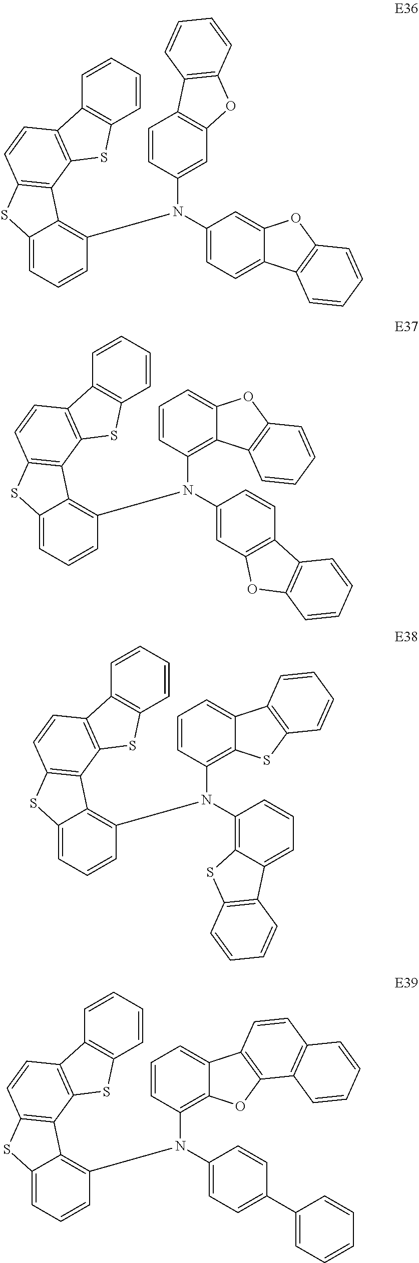 [SMILES: c1ccc2c(c1)oc1cc(N(c3ccc4c(c3)oc3ccccc34)c3cccc4sc5ccc6c7ccccc7sc6c5c34)ccc12 ; c1ccc2c(c1)oc1cc(N(c3cccc4oc5ccccc5c34)c3cccc4sc5ccc6c7ccccc7sc6c5c34)ccc12 ; c1ccc2c(c1)sc1c(N(c3cccc4c3sc3ccccc34)c3cccc4sc5ccc6c7ccccc7sc6c5c34)cccc12 ; c1ccc(-c2ccc(N(c3cccc4c3oc3c5ccccc5ccc43)c3cccc4sc5ccc6c7ccccc7sc6c5c34)cc2)cc1]

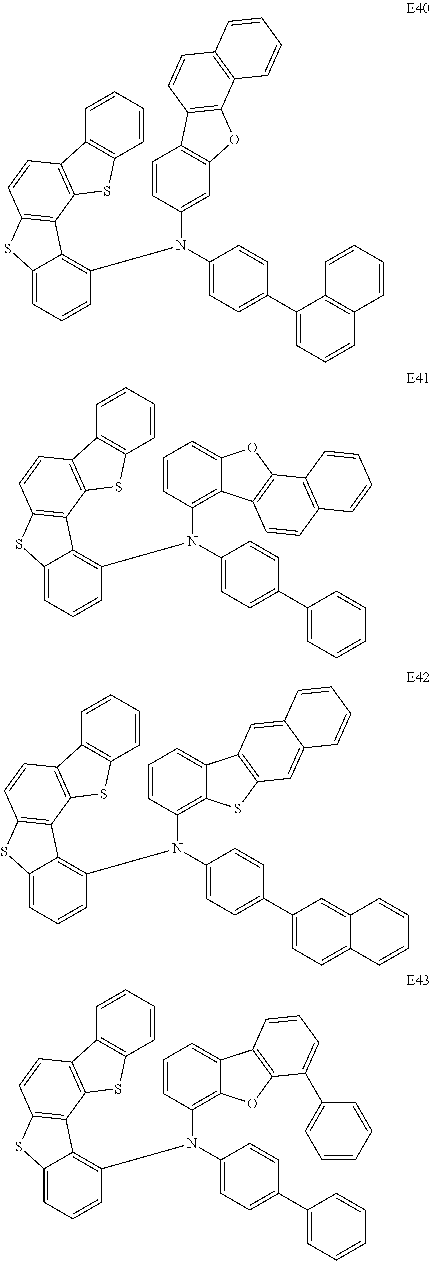

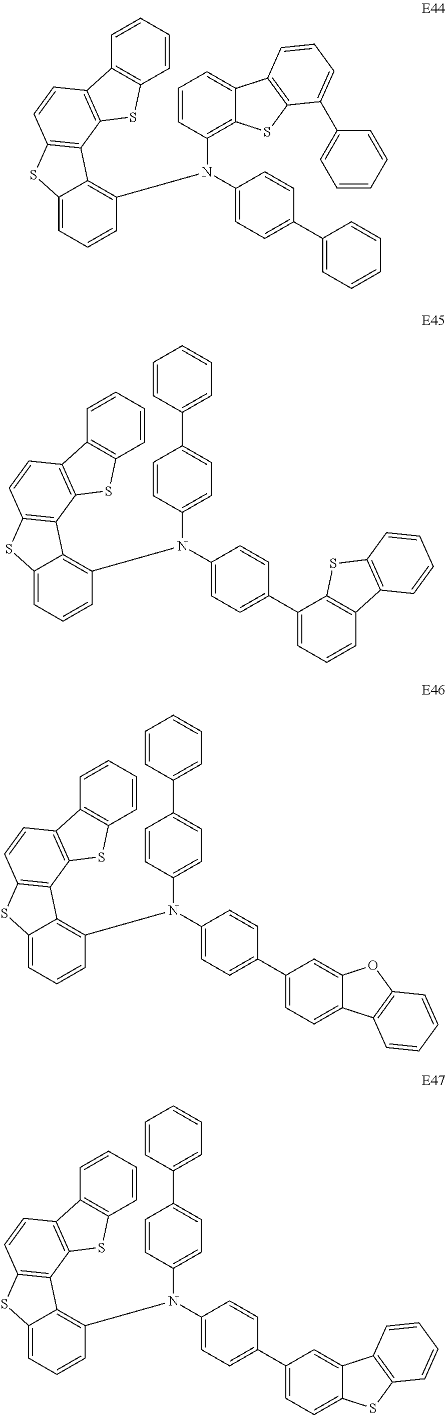 [SMILES: c1ccc(-c2ccc(N(c3cccc4c3sc3c(-c5ccccc5)cccc34)c3cccc4sc5ccc6c7ccccc7sc6c5c34)cc2)cc1 ; c1ccc(-c2ccc(N(c3ccc(-c4cccc5c4sc4ccccc45)cc3)c3cccc4sc5ccc6c7ccccc7sc6c5c34)cc2)cc1 ; c1ccc(-c2ccc(N(c3ccc(-c4ccc5c(c4)oc4ccccc45)cc3)c3cccc4sc5ccc6c7ccccc7sc6c5c34)cc2)cc1 ; c1ccc(-c2ccc(N(c3ccc(-c4ccc5sc6ccccc6c5c4)cc3)c3cccc4sc5ccc6c7ccccc7sc6c5c34)cc2)cc1]

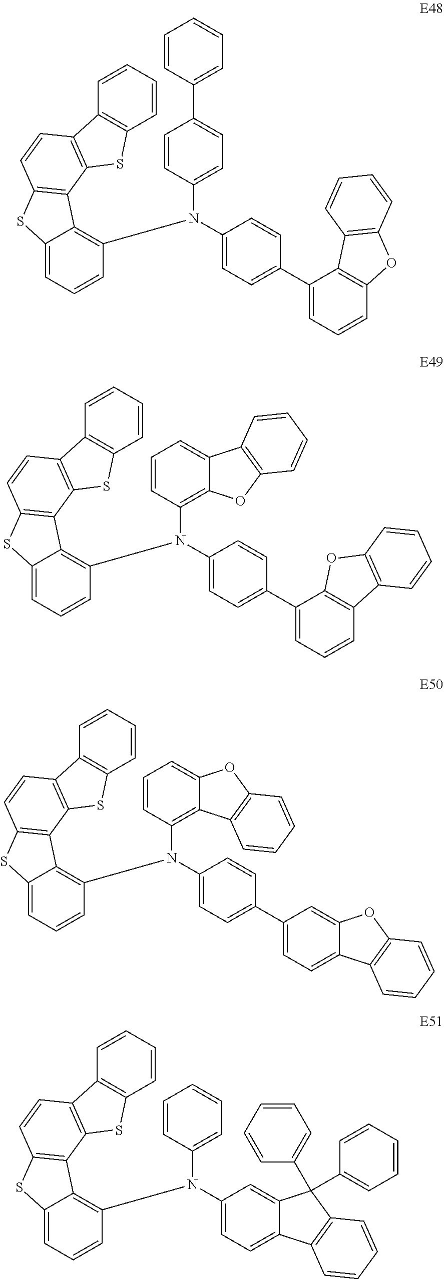 [SMILES: c1ccc(-c2ccc(N(c3ccc(-c4cccc5oc6ccccc6c45)cc3)c3cccc4sc5ccc6c7ccccc7sc6c5c34)cc2)cc1 ; c1ccc2c(c1)oc1c(-c3ccc(N(c4cccc5c4oc4ccccc45)c4cccc5sc6ccc7c8ccccc8sc7c6c45)cc3)cccc12 ; c1ccc2c(c1)oc1cc(-c3ccc(N(c4cccc5oc6ccccc6c45)c4cccc5sc6ccc7c8ccccc8sc7c6c45)cc3)ccc12 ; c1ccc(N(c2ccc3c(c2)C(c2ccccc2)(c2ccccc2)c2ccccc2-3)c2cccc3sc4ccc5c6ccccc6sc5c4c23)cc1]

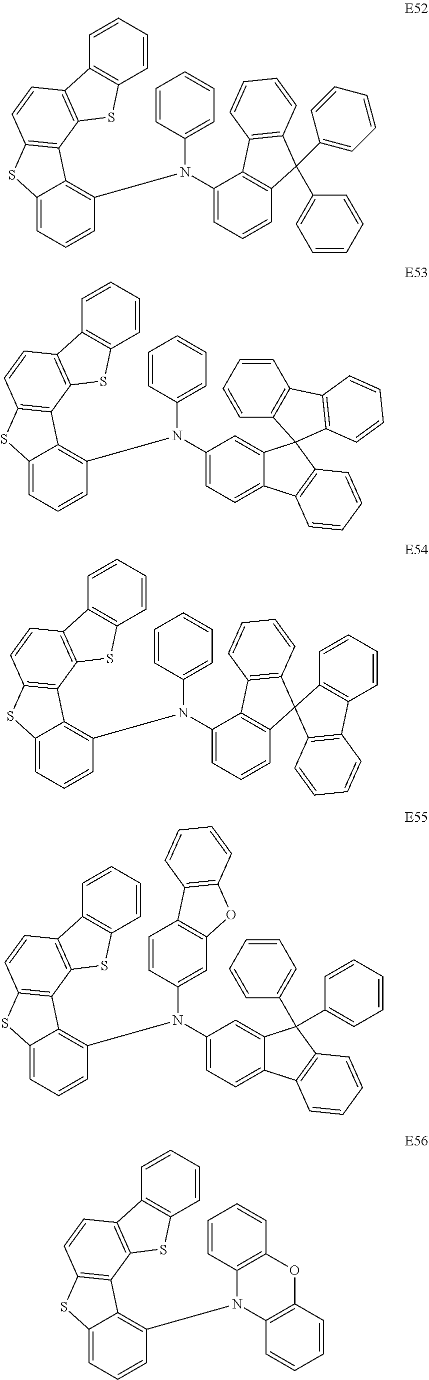 [SMILES: c1ccc(N(c2cccc3c2-c2ccccc2C3(c2ccccc2)c2ccccc2)c2cccc3sc4ccc5c6ccccc6sc5c4c23)cc1 ; c1ccc(N(c2ccc3c(c2)C2(c4ccccc4-c4ccccc42)c2ccccc2-3)c2cccc3sc4ccc5c6ccccc6sc5c4c23)cc1 ; c1ccc(N(c2cccc3c2-c2ccccc2C32c3ccccc3-c3ccccc32)c2cccc3sc4ccc5c6ccccc6sc5c4c23)cc1 ; c1ccc(C2(c3ccccc3)c3ccccc3-c3ccc(N(c4ccc5c(c4)oc4ccccc45)c4cccc5sc6ccc7c8ccccc8sc7c6c45)cc32)cc1 ; c1ccc2c(c1)Oc1ccccc1N2c1cccc2sc3ccc4c5ccccc5sc4c3c12]

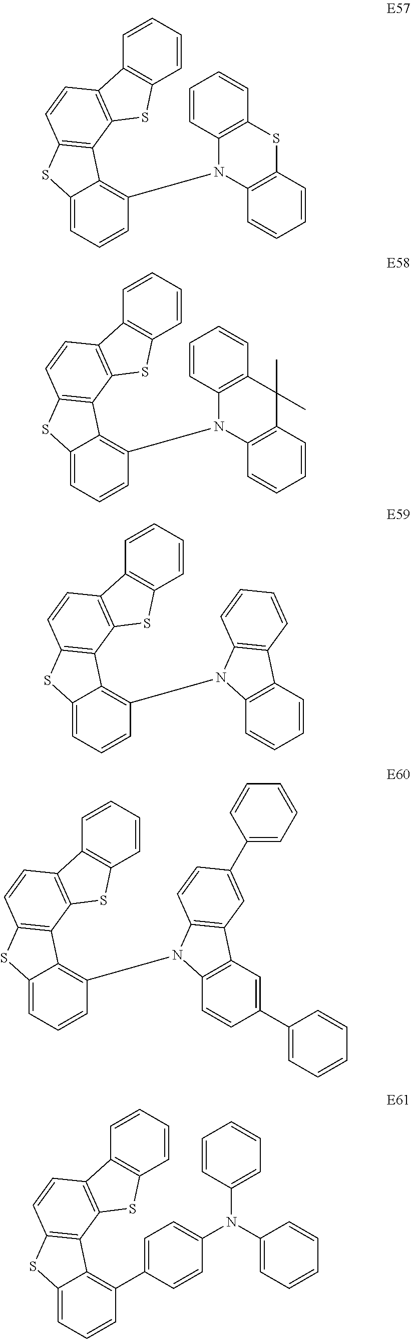

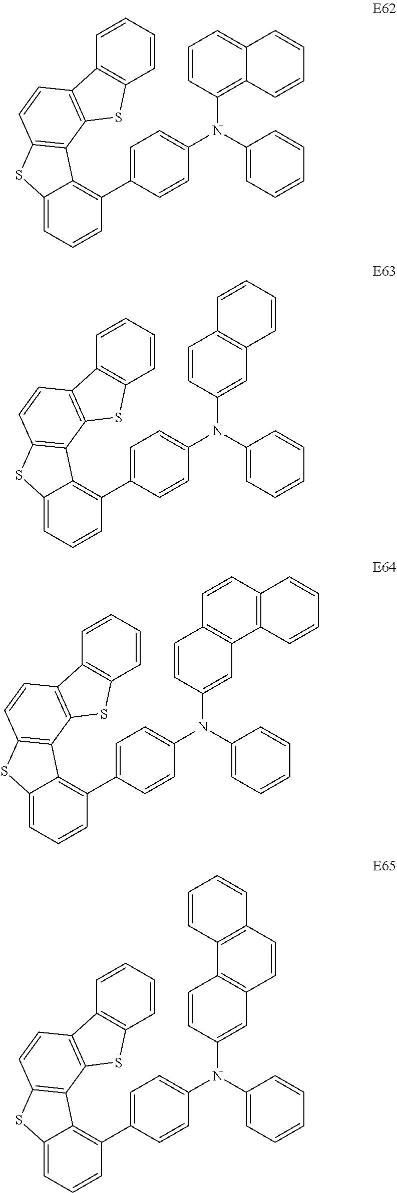 [SMILES: c1ccc(N(c2ccc(-c3cccc4sc5ccc6c7ccccc7sc6c5c34)cc2)c2cccc3ccccc23)cc1 ; c1ccc(N(c2ccc(-c3cccc4sc5ccc6c7ccccc7sc6c5c34)cc2)c2ccc3ccccc3c2)cc1 ; c1ccc(N(c2ccc(-c3cccc4sc5ccc6c7ccccc7sc6c5c34)cc2)c2ccc3ccc4ccccc4c3c2)cc1 ; c1ccc(N(c2ccc(-c3cccc4sc5ccc6c7ccccc7sc6c5c34)cc2)c2ccc3c(ccc4ccccc43)c2)cc1]

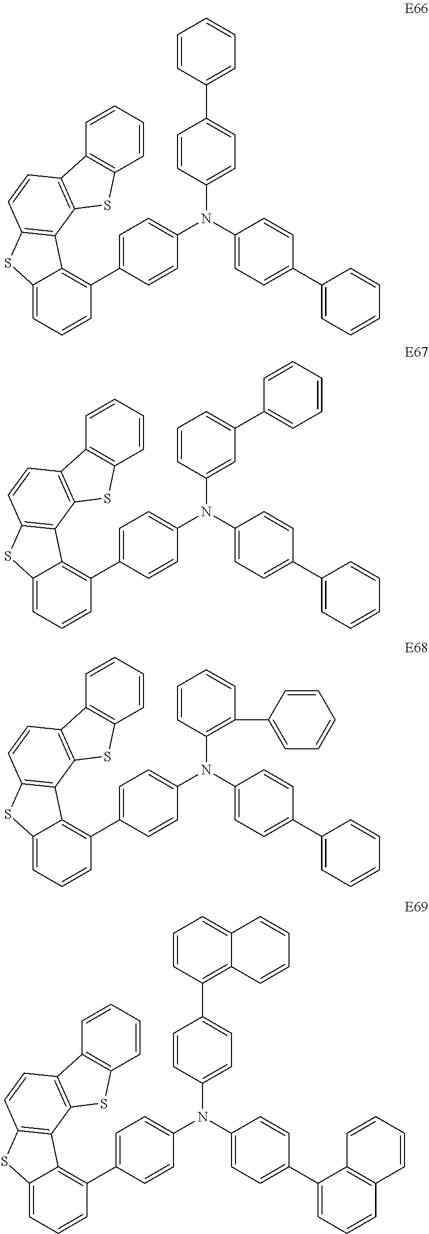 [SMILES: c1ccc(-c2ccc(N(c3ccc(-c4ccccc4)cc3)c3ccc(-c4cccc5sc6ccc7c8ccccc8sc7c6c45)cc3)cc2)cc1 ; c1ccc(-c2ccc(N(c3ccc(-c4cccc5sc6ccc7c8ccccc8sc7c6c45)cc3)c3cccc(-c4ccccc4)c3)cc2)cc1 ; c1ccc(-c2ccc(N(c3ccc(-c4cccc5sc6ccc7c8ccccc8sc7c6c45)cc3)c3ccccc3-c3ccccc3)cc2)cc1 ; c1ccc2c(-c3ccc(N(c4ccc(-c5cccc6ccccc56)cc4)c4ccc(-c5cccc6sc7ccc8c9ccccc9sc8c7c56)cc4)cc3)cccc2c1]

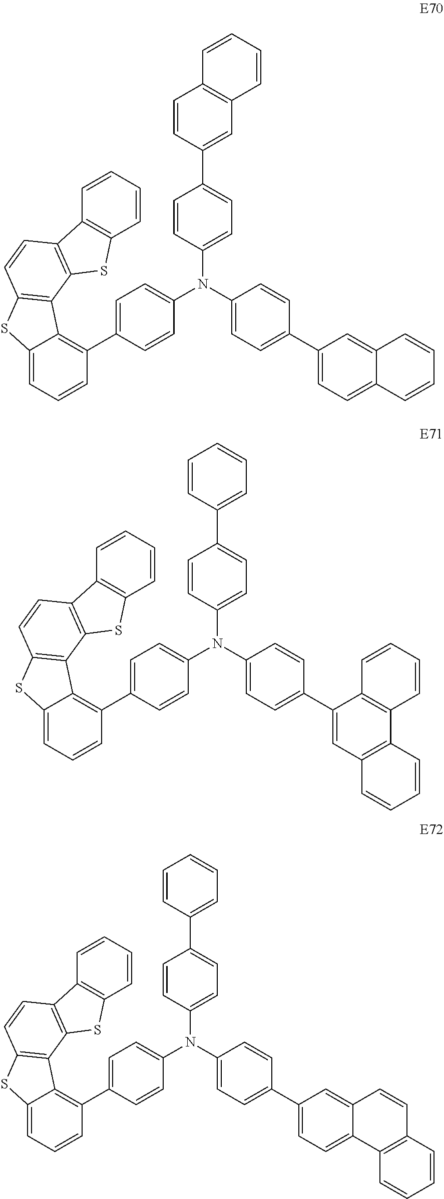 [SMILES: c1ccc2cc(-c3ccc(N(c4ccc(-c5ccc6ccccc6c5)cc4)c4ccc(-c5cccc6sc7ccc8c9ccccc9sc8c7c56)cc4)cc3)ccc2c1 ; c1ccc(-c2ccc(N(c3ccc(-c4cc5ccccc5c5ccccc45)cc3)c3ccc(-c4cccc5sc6ccc7c8ccccc8sc7c6c45)cc3)cc2)cc1 ; c1ccc(-c2ccc(N(c3ccc(-c4ccc5c(ccc6ccccc65)c4)cc3)c3ccc(-c4cccc5sc6ccc7c8ccccc8sc7c6c45)cc3)cc2)cc1]

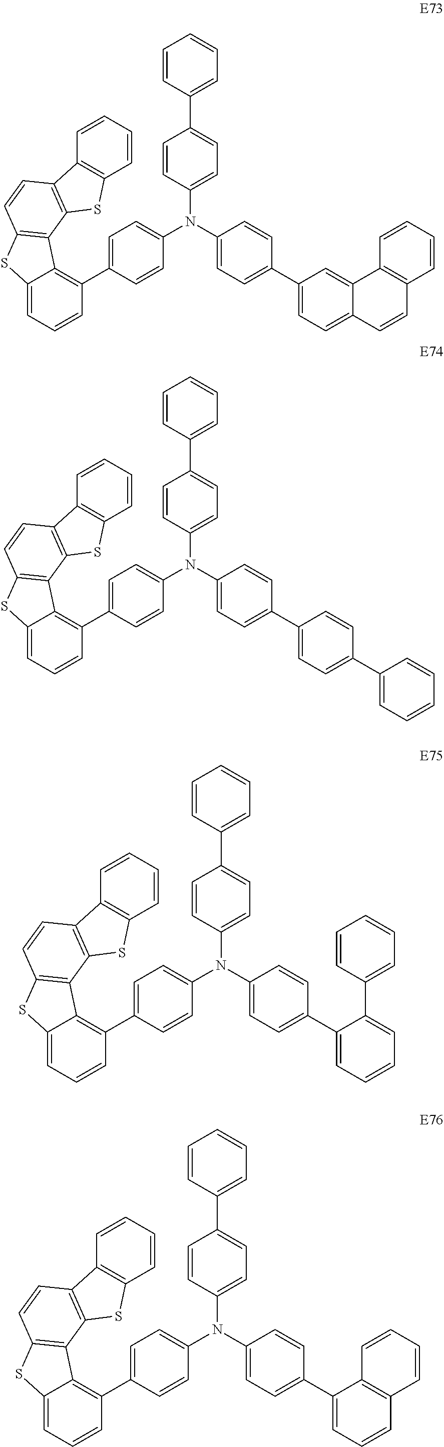 [SMILES: c1ccc(-c2ccc(N(c3ccc(-c4ccc5ccc6ccccc6c5c4)cc3)c3ccc(-c4cccc5sc6ccc7c8ccccc8sc7c6c45)cc3)cc2)cc1 ; c1ccc(-c2ccc(-c3ccc(N(c4ccc(-c5ccccc5)cc4)c4ccc(-c5cccc6sc7ccc8c9ccccc9sc8c7c56)cc4)cc3)cc2)cc1 ; c1ccc(-c2ccc(N(c3ccc(-c4ccccc4-c4ccccc4)cc3)c3ccc(-c4cccc5sc6ccc7c8ccccc8sc7c6c45)cc3)cc2)cc1 ; c1ccc(-c2ccc(N(c3ccc(-c4cccc5ccccc45)cc3)c3ccc(-c4cccc5sc6ccc7c8ccccc8sc7c6c45)cc3)cc2)cc1]

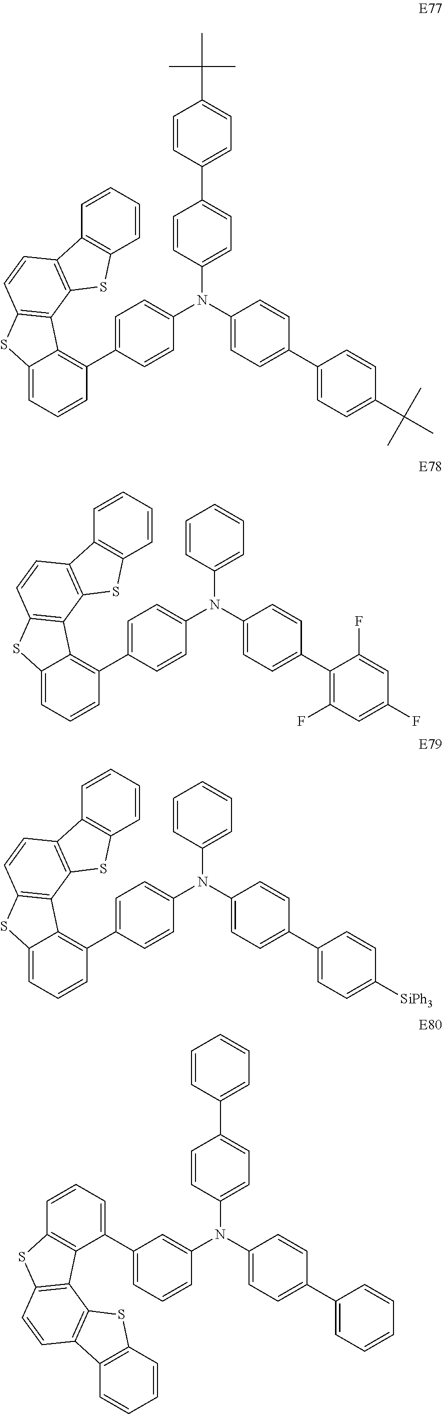

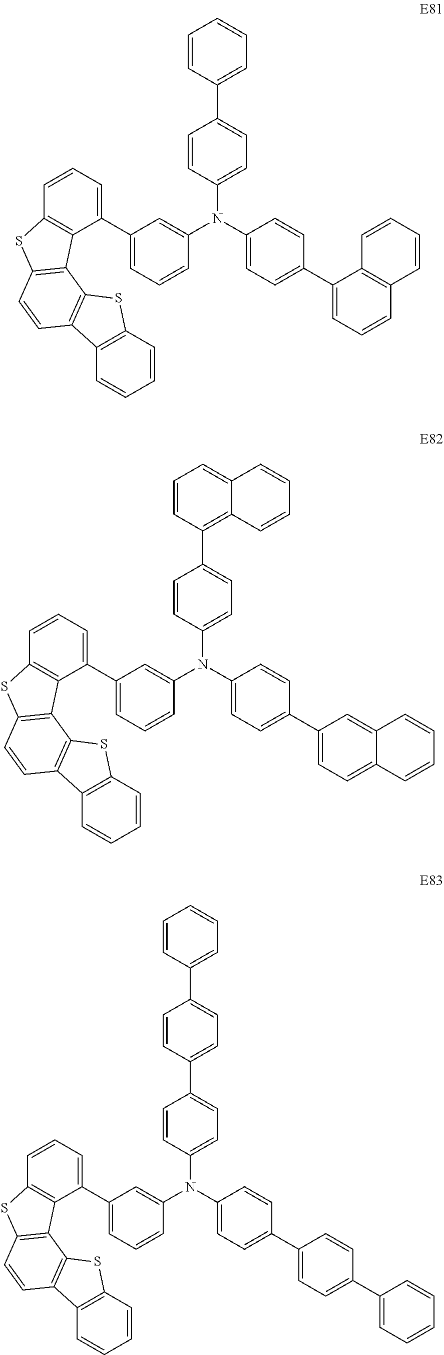 [SMILES: c1ccc(-c2ccc(N(c3ccc(-c4cccc5ccccc45)cc3)c3cccc(-c4cccc5sc6ccc7c8ccccc8sc7c6c45)c3)cc2)cc1 ; c1cc(-c2cccc3sc4ccc5c6ccccc6sc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6sc7ccc8c9ccccc9sc8c7c56)c4)cc3)cc2)cc1]

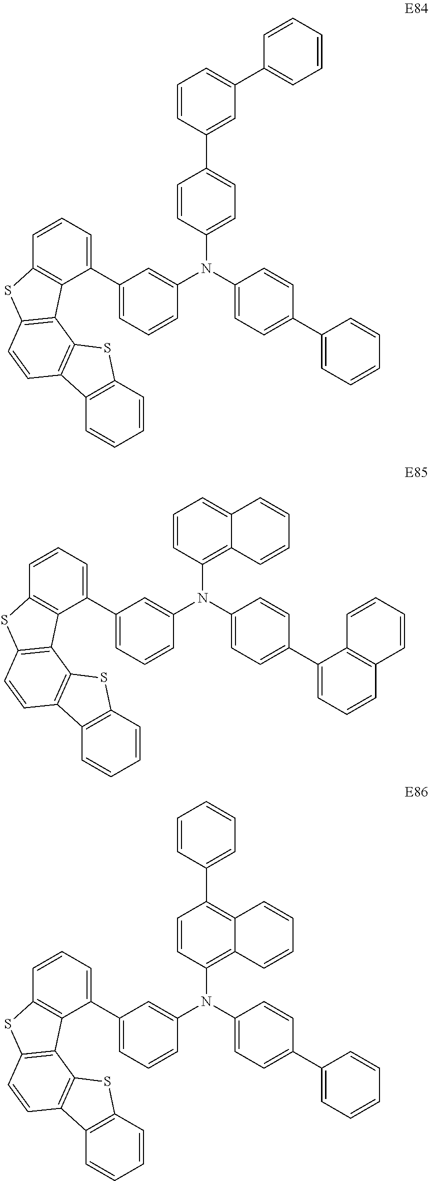 [SMILES: c1ccc(-c2ccc(N(c3ccc(-c4cccc(-c5ccccc5)c4)cc3)c3cccc(-c4cccc5sc6ccc7c8ccccc8sc7c6c45)c3)cc2)cc1 ; c1cc(-c2cccc3sc4ccc5c6ccccc6sc5c4c23)cc(N(c2ccc(-c3cccc4ccccc34)cc2)c2cccc3ccccc23)c1 ; c1ccc(-c2ccc(N(c3cccc(-c4cccc5sc6ccc7c8ccccc8sc7c6c45)c3)c3ccc(-c4ccccc4)c4ccccc34)cc2)cc1]

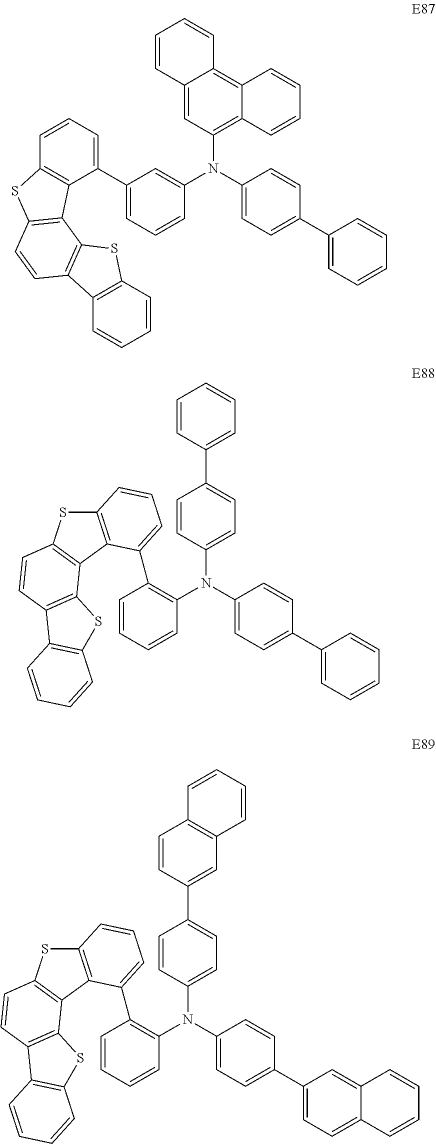 [SMILES: c1ccc(-c2ccc(N(c3cccc(-c4cccc5sc6ccc7c8ccccc8sc7c6c45)c3)c3cc4ccccc4c4ccccc34)cc2)cc1 ; c1ccc(-c2ccc(N(c3ccc(-c4ccccc4)cc3)c3ccccc3-c3cccc4sc5ccc6c7ccccc7sc6c5c34)cc2)cc1 ; c1ccc(N(c2ccc(-c3ccc4ccccc4c3)cc2)c2ccc(-c3ccc4ccccc4c3)cc2)c(-c2cccc3sc4ccc5c6ccccc6sc5c4c23)c1]

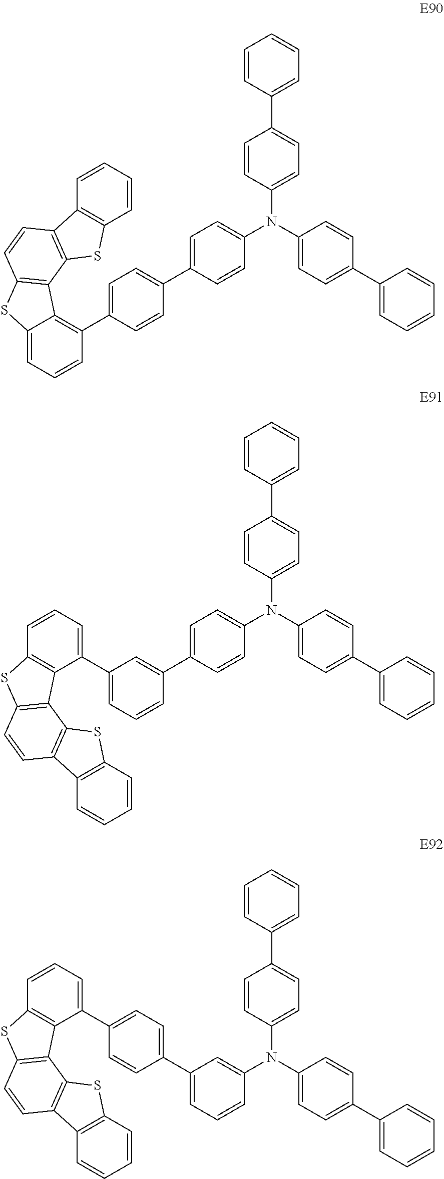 [SMILES: c1ccc(-c2ccc(N(c3ccc(-c4ccccc4)cc3)c3ccc(-c4ccc(-c5cccc6sc7ccc8c9ccccc9sc8c7c56)cc4)cc3)cc2)cc1 ; c1ccc(-c2ccc(N(c3ccc(-c4ccccc4)cc3)c3ccc(-c4cccc(-c5cccc6sc7ccc8c9ccccc9sc8c7c56)c4)cc3)cc2)cc1 ; c1ccc(-c2ccc(N(c3ccc(-c4ccccc4)cc3)c3cccc(-c4ccc(-c5cccc6sc7ccc8c9ccccc9sc8c7c56)cc4)c3)cc2)cc1]

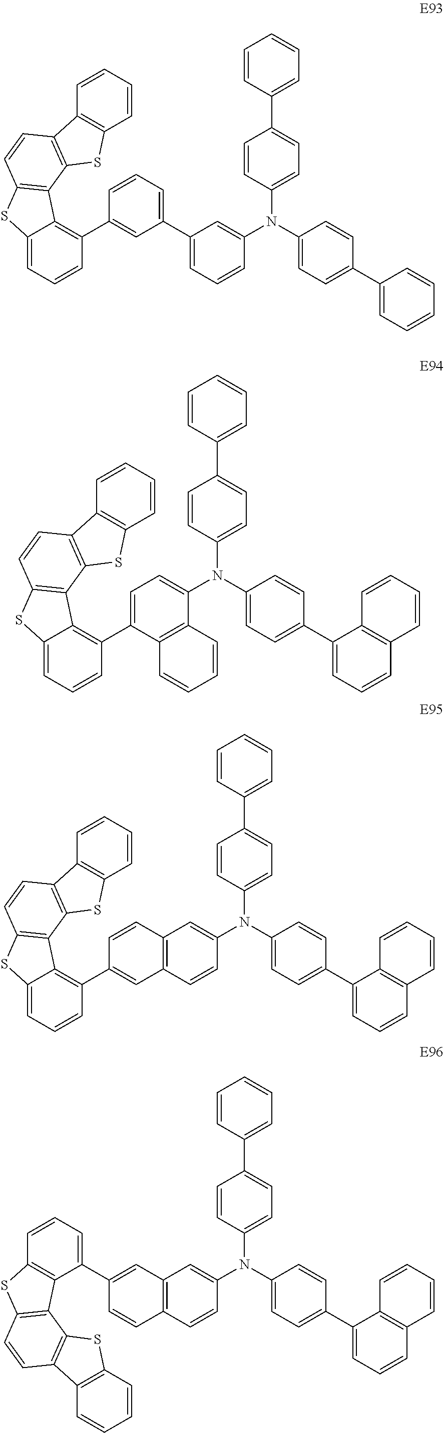 [SMILES: c1ccc(-c2ccc(N(c3ccc(-c4ccccc4)cc3)c3cccc(-c4cccc(-c5cccc6sc7ccc8c9ccccc9sc8c7c56)c4)c3)cc2)cc1 ; c1ccc(-c2ccc(N(c3ccc(-c4cccc5ccccc45)cc3)c3ccc(-c4cccc5sc6ccc7c8ccccc8sc7c6c45)c4ccccc34)cc2)cc1 ; c1ccc(-c2ccc(N(c3ccc(-c4cccc5ccccc45)cc3)c3ccc4cc(-c5cccc6sc7ccc8c9ccccc9sc8c7c56)ccc4c3)cc2)cc1 ; c1ccc(-c2ccc(N(c3ccc(-c4cccc5ccccc45)cc3)c3ccc4ccc(-c5cccc6sc7ccc8c9ccccc9sc8c7c56)cc4c3)cc2)cc1]

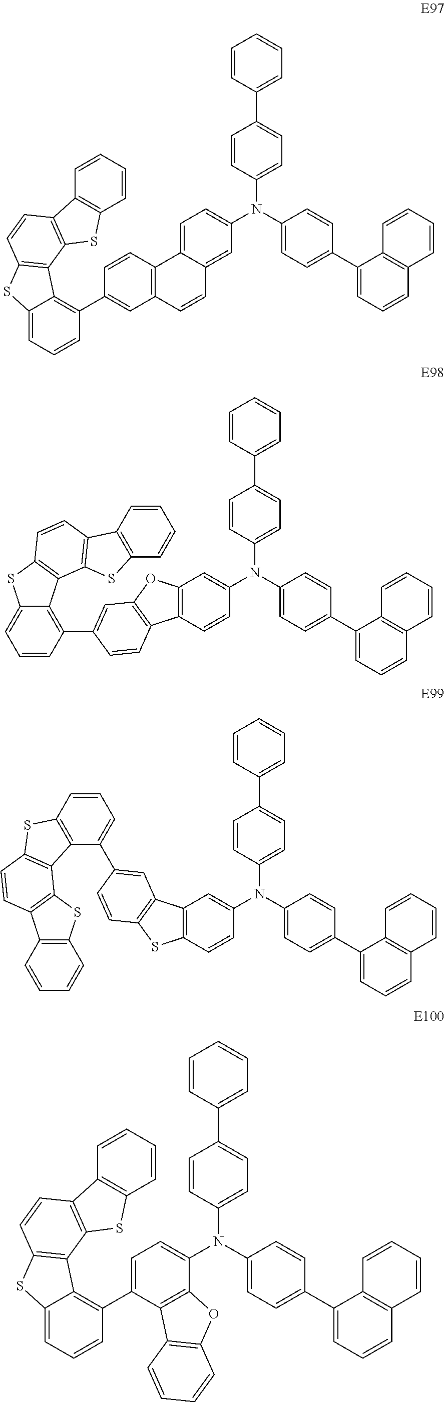 [SMILES: c1ccc(-c2ccc(N(c3ccc(-c4cccc5ccccc45)cc3)c3ccc4c(ccc5cc(-c6cccc7sc8ccc9c%10ccccc%10sc9c8c67)ccc54)c3)cc2)cc1 ; c1ccc(-c2ccc(N(c3ccc(-c4cccc5ccccc45)cc3)c3ccc4c(c3)oc3cc(-c5cccc6sc7ccc8c9ccccc9sc8c7c56)ccc34)cc2)cc1 ; c1ccc(-c2ccc(N(c3ccc(-c4cccc5ccccc45)cc3)c3ccc4sc5ccc(-c6cccc7sc8ccc9c%10ccccc%10sc9c8c67)cc5c4c3)cc2)cc1 ; c1ccc(-c2ccc(N(c3ccc(-c4cccc5ccccc45)cc3)c3ccc(-c4cccc5sc6ccc7c8ccccc8sc7c6c45)c4c3oc3ccccc34)cc2)cc1]

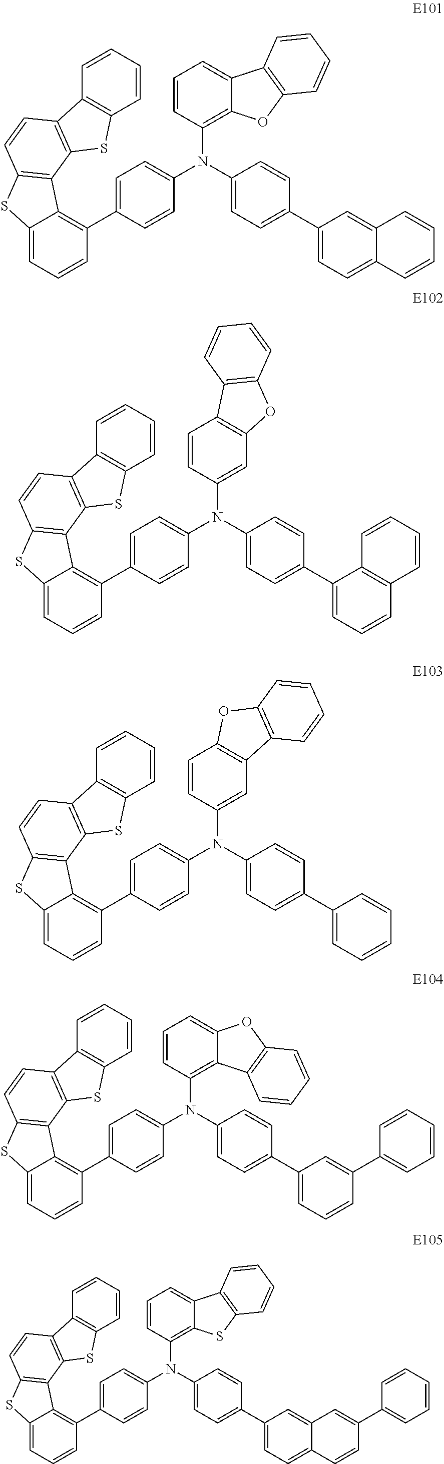 [SMILES: c1ccc2cc(-c3ccc(N(c4ccc(-c5cccc6sc7ccc8c9ccccc9sc8c7c56)cc4)c4cccc5c4oc4ccccc45)cc3)ccc2c1 ; c1ccc2c(-c3ccc(N(c4ccc(-c5cccc6sc7ccc8c9ccccc9sc8c7c56)cc4)c4ccc5c(c4)oc4ccccc45)cc3)cccc2c1 ; c1ccc(-c2ccc(N(c3ccc(-c4cccc5sc6ccc7c8ccccc8sc7c6c45)cc3)c3ccc4oc5ccccc5c4c3)cc2)cc1 ; c1ccc(-c2cccc(-c3ccc(N(c4ccc(-c5cccc6sc7ccc8c9ccccc9sc8c7c56)cc4)c4cccc5oc6ccccc6c45)cc3)c2)cc1 ; c1ccc(-c2ccc3ccc(-c4ccc(N(c5ccc(-c6cccc7sc8ccc9c%10ccccc%10sc9c8c67)cc5)c5cccc6c5sc5ccccc56)cc4)cc3c2)cc1]

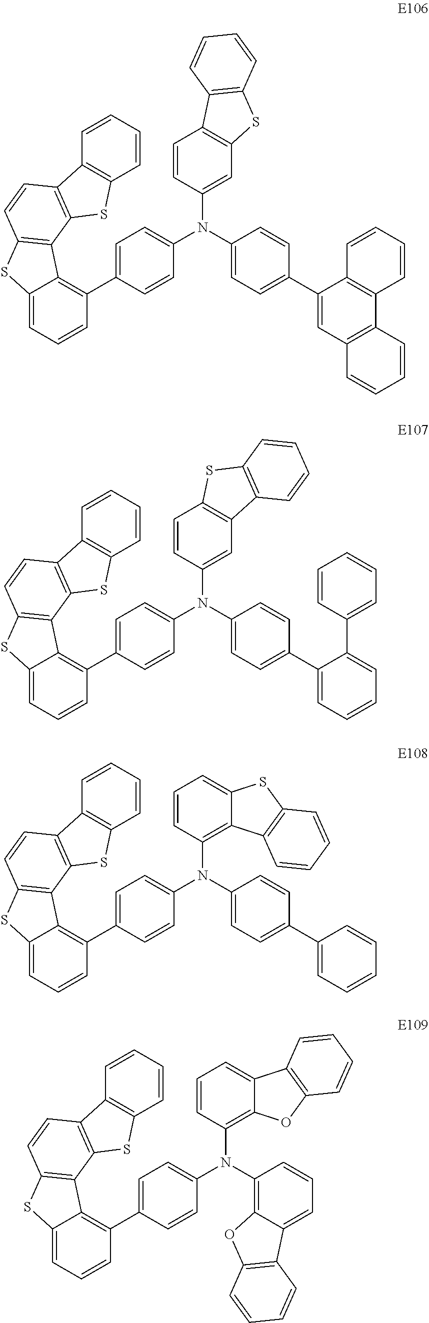

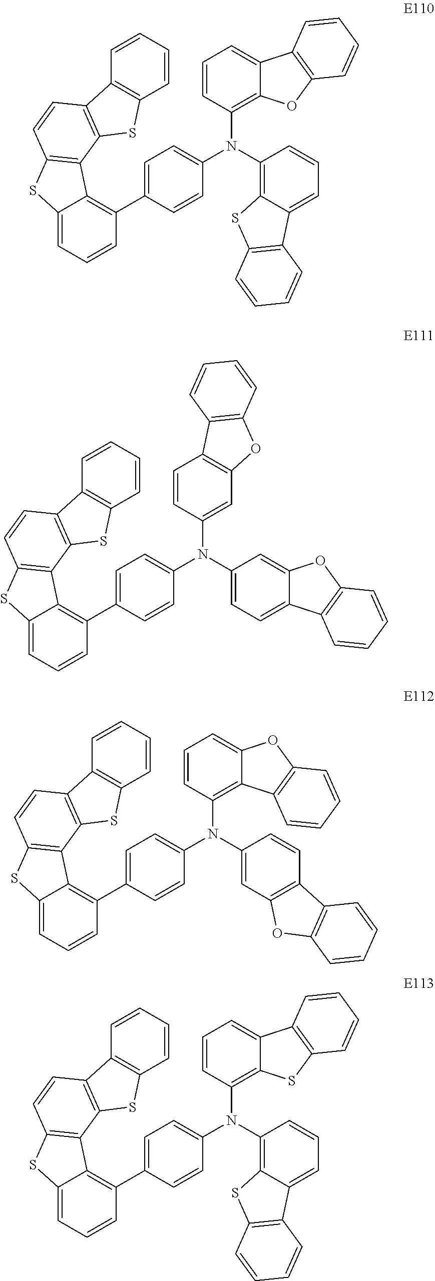

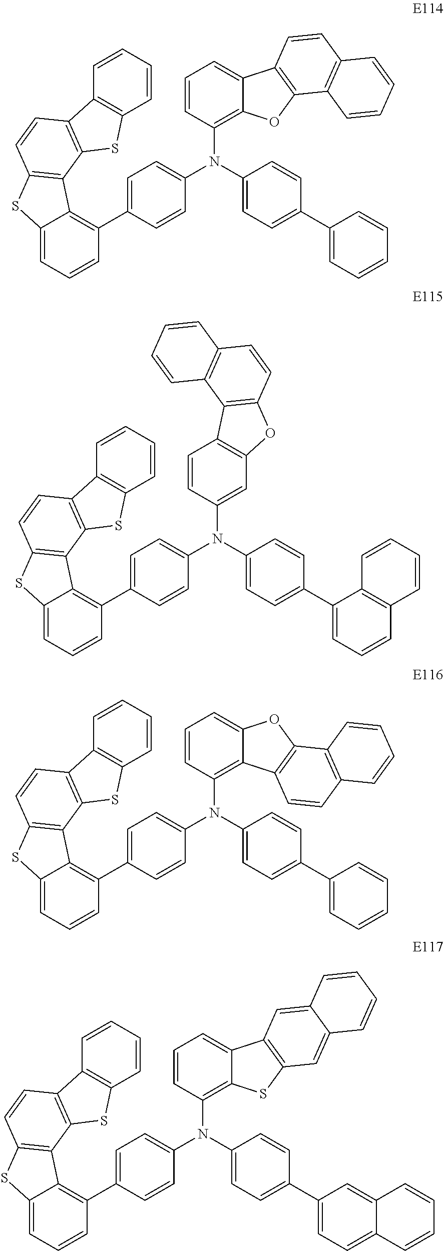

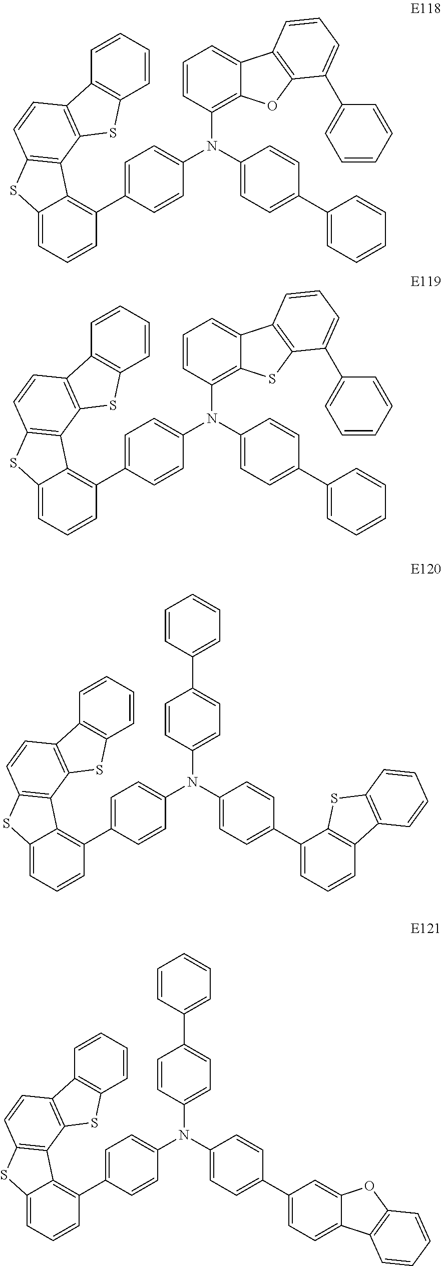 [SMILES: c1ccc(-c2ccc(N(c3ccc(-c4cccc5sc6ccc7c8ccccc8sc7c6c45)cc3)c3cccc4c3oc3c(-c5ccccc5)cccc34)cc2)cc1 ; c1ccc(-c2ccc(N(c3ccc(-c4cccc5sc6ccc7c8ccccc8sc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5sc6ccc7c8ccccc8sc7c6c45)cc3)cc2)cc1 ; c1ccc(-c2ccc(N(c3ccc(-c4ccc5c(c4)oc4ccccc45)cc3)c3ccc(-c4cccc5sc6ccc7c8ccccc8sc7c6c45)cc3)cc2)cc1]

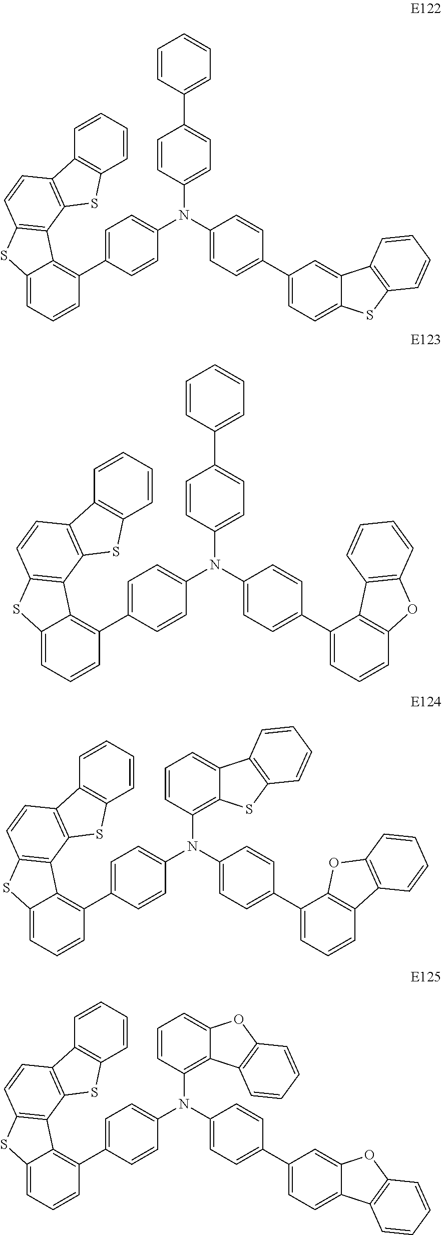 [SMILES: c1ccc(-c2ccc(N(c3ccc(-c4ccc5sc6ccccc6c5c4)cc3)c3ccc(-c4cccc5sc6ccc7c8ccccc8sc7c6c45)cc3)cc2)cc1 ; c1ccc(-c2ccc(N(c3ccc(-c4cccc5oc6ccccc6c45)cc3)c3ccc(-c4cccc5sc6ccc7c8ccccc8sc7c6c45)cc3)cc2)cc1 ; c1ccc2c(c1)oc1c(-c3ccc(N(c4ccc(-c5cccc6sc7ccc8c9ccccc9sc8c7c56)cc4)c4cccc5c4sc4ccccc45)cc3)cccc12 ; c1ccc2c(c1)oc1cc(-c3ccc(N(c4ccc(-c5cccc6sc7ccc8c9ccccc9sc8c7c56)cc4)c4cccc5oc6ccccc6c45)cc3)ccc12]

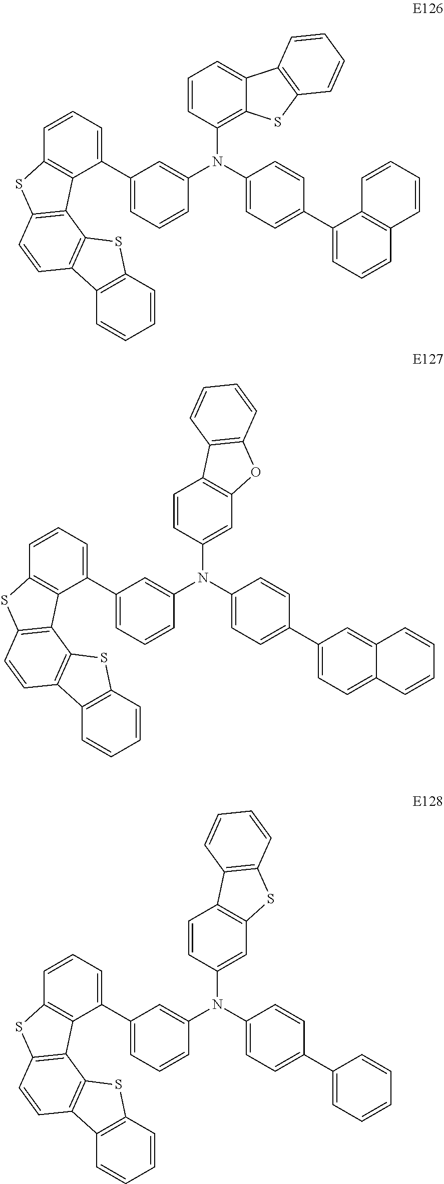 [SMILES: c1cc(-c2cccc3sc4ccc5c6ccccc6sc5c4c23)cc(N(c2ccc(-c3cccc4ccccc34)cc2)c2cccc3c2sc2ccccc23)c1 ; c1cc(-c2cccc3sc4ccc5c6ccccc6sc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5sc6ccc7c8ccccc8sc7c6c45)c3)c3ccc4c(c3)sc3ccccc34)cc2)cc1]

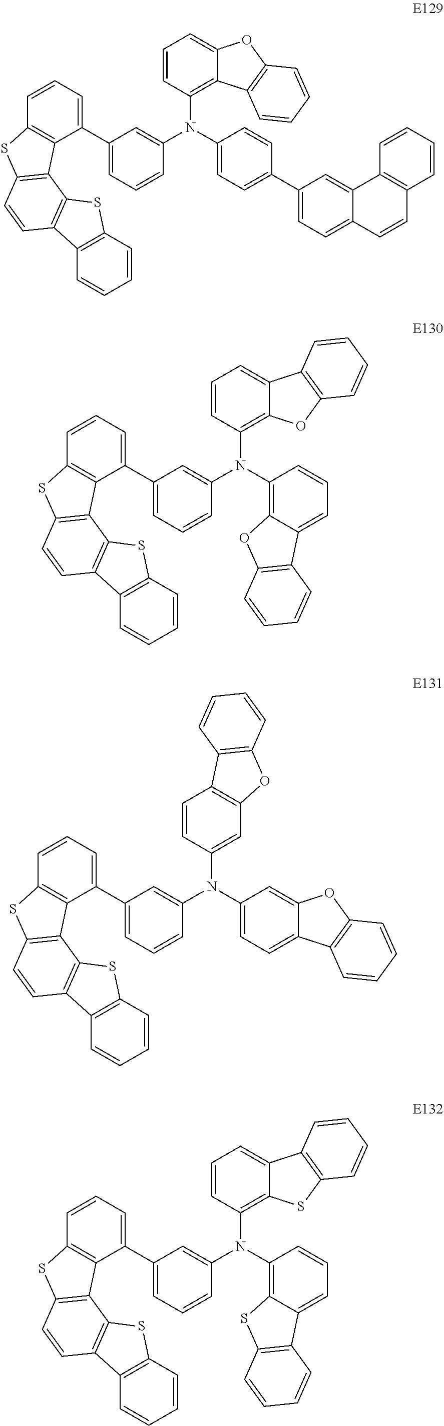 [SMILES: c1cc(-c2cccc3sc4ccc5c6ccccc6sc5c4c23)cc(N(c2ccc(-c3ccc4ccc5ccccc5c4c3)cc2)c2cccc3oc4ccccc4c23)c1 ; c1cc(-c2cccc3sc4ccc5c6ccccc6sc5c4c23)cc(N(c2cccc3c2oc2ccccc23)c2cccc3c2oc2ccccc23)c1 ; c1cc(-c2cccc3sc4ccc5c6ccccc6sc5c4c23)cc(N(c2ccc3c(c2)oc2ccccc23)c2ccc3c(c2)oc2ccccc23)c1 ; c1cc(-c2cccc3sc4ccc5c6ccccc6sc5c4c23)cc(N(c2cccc3c2sc2ccccc23)c2cccc3c2sc2ccccc23)c1]

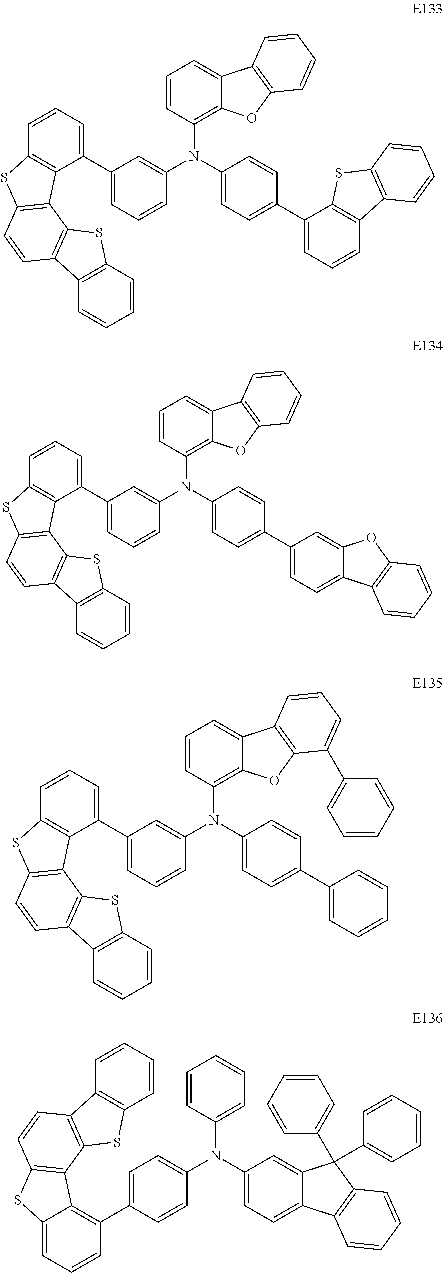

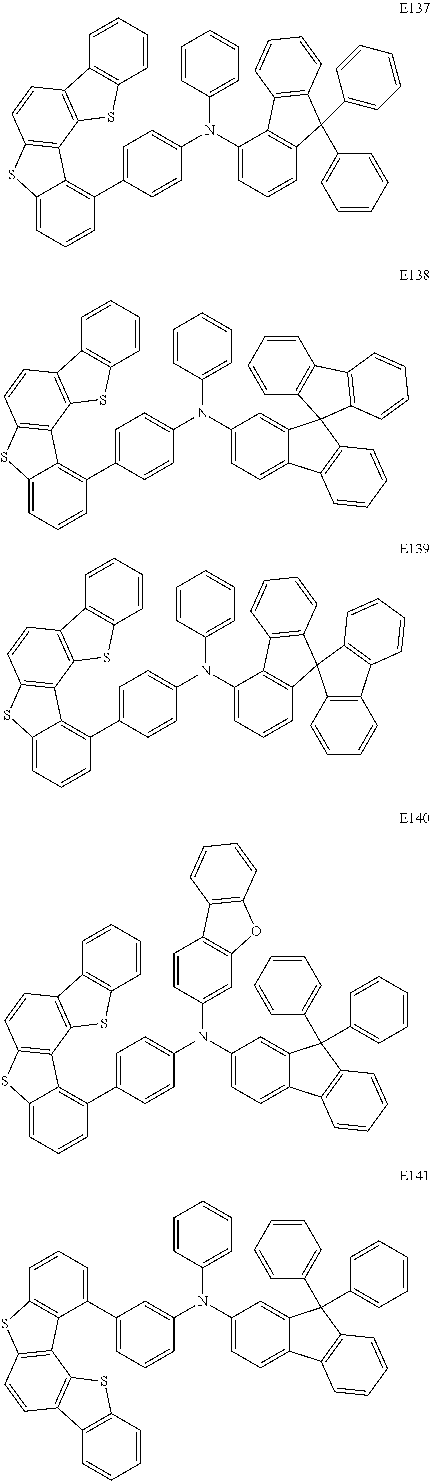 [SMILES: c1ccc(N(c2ccc(-c3cccc4sc5ccc6c7ccccc7sc6c5c34)cc2)c2cccc3c2-c2ccccc2C3(c2ccccc2)c2ccccc2)cc1 ; c1ccc(N(c2ccc(-c3cccc4sc5ccc6c7ccccc7sc6c5c34)cc2)c2ccc3c(c2)C2(c4ccccc4-c4ccccc42)c2ccccc2-3)cc1 ; c1ccc(N(c2ccc(-c3cccc4sc5ccc6c7ccccc7sc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6sc7ccc8c9ccccc9sc8c7c56)cc4)c4ccc5c(c4)oc4ccccc45)cc32)cc1 ; c1ccc(N(c2cccc(-c3cccc4sc5ccc6c7ccccc7sc6c5c34)c2)c2ccc3c(c2)C(c2ccccc2)(c2ccccc2)c2ccccc2-3)cc1]

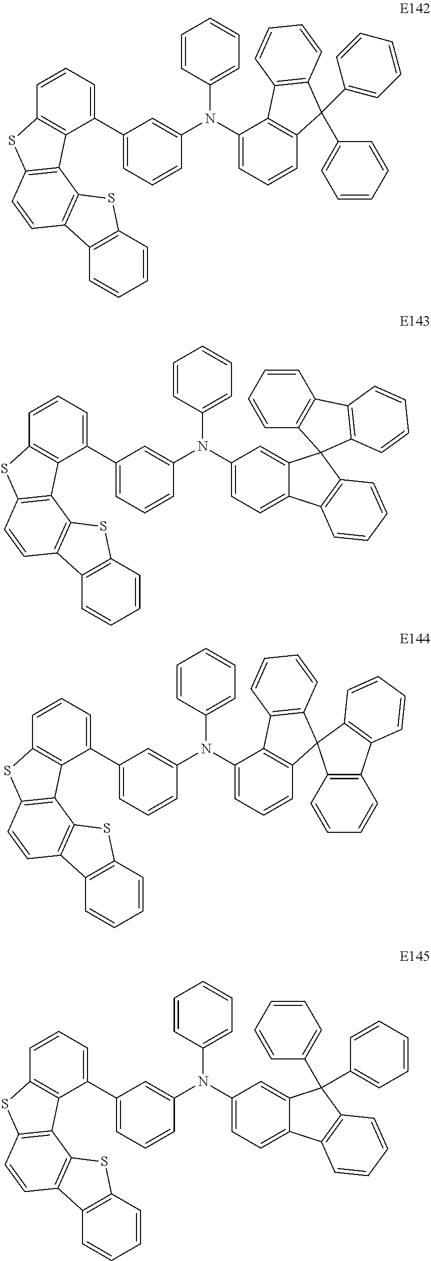 [SMILES: c1ccc(N(c2cccc(-c3cccc4sc5ccc6c7ccccc7sc6c5c34)c2)c2cccc3c2-c2ccccc2C3(c2ccccc2)c2ccccc2)cc1 ; c1ccc(N(c2cccc(-c3cccc4sc5ccc6c7ccccc7sc6c5c34)c2)c2ccc3c(c2)C2(c4ccccc4-c4ccccc42)c2ccccc2-3)cc1 ; c1ccc(N(c2cccc(-c3cccc4sc5ccc6c7ccccc7sc6c5c34)c2)c2cccc3c2-c2ccccc2C32c3ccccc3-c3ccccc32)cc1 ; c1ccc(N(c2cccc(-c3cccc4sc5ccc6c7ccccc7sc6c5c34)c2)c2ccc3c(c2)C(c2ccccc2)(c2ccccc2)c2ccccc2-3)cc1]

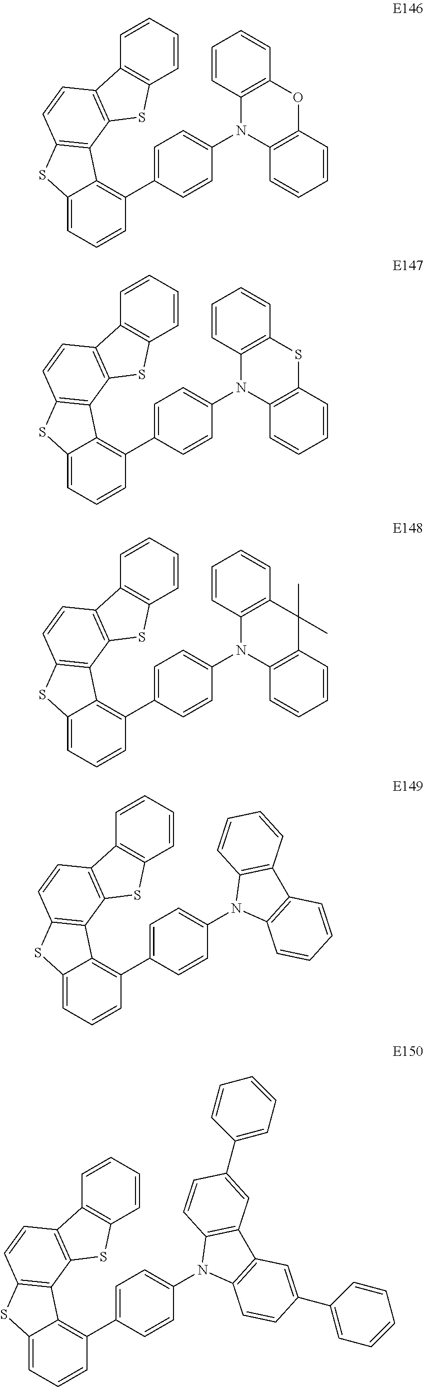 [SMILES: c1ccc2c(c1)Oc1ccccc1N2c1ccc(-c2cccc3sc4ccc5c6ccccc6sc5c4c23)cc1 ; c1ccc2c(c1)Sc1ccccc1N2c1ccc(-c2cccc3sc4ccc5c6ccccc6sc5c4c23)cc1 ; CC1(C)c2ccccc2N(c2ccc(-c3cccc4sc5ccc6c7ccccc7sc6c5c34)cc2)c2ccccc21 ; c1ccc2c(c1)sc1c2ccc2sc3cccc(-c4ccc(-n5c6ccccc6c6ccccc65)cc4)c3c21 ; c1ccc(-c2ccc3c(c2)c2cc(-c4ccccc4)ccc2n3-c2ccc(-c3cccc4sc5ccc6c7ccccc7sc6c5c34)cc2)cc1]

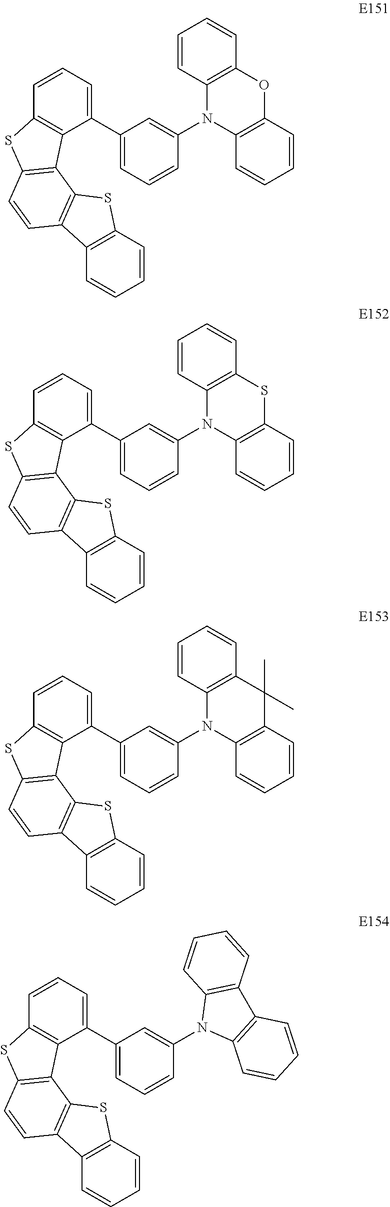 [SMILES: c1cc(-c2cccc3sc4ccc5c6ccccc6sc5c4c23)cc(N2c3ccccc3Oc3ccccc32)c1 ; c1cc(-c2cccc3sc4ccc5c6ccccc6sc5c4c23)cc(N2c3ccccc3Sc3ccccc32)c1 ; CC1(C)c2ccccc2N(c2cccc(-c3cccc4sc5ccc6c7ccccc7sc6c5c34)c2)c2ccccc21 ; c1cc(-c2cccc3sc4ccc5c6ccccc6sc5c4c23)cc(-n2c3ccccc3c3ccccc32)c1]

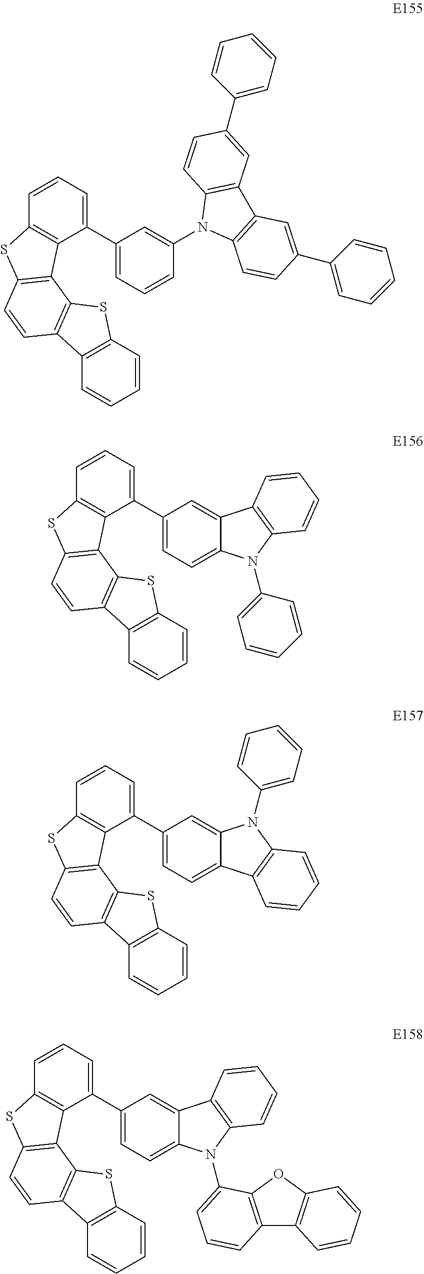

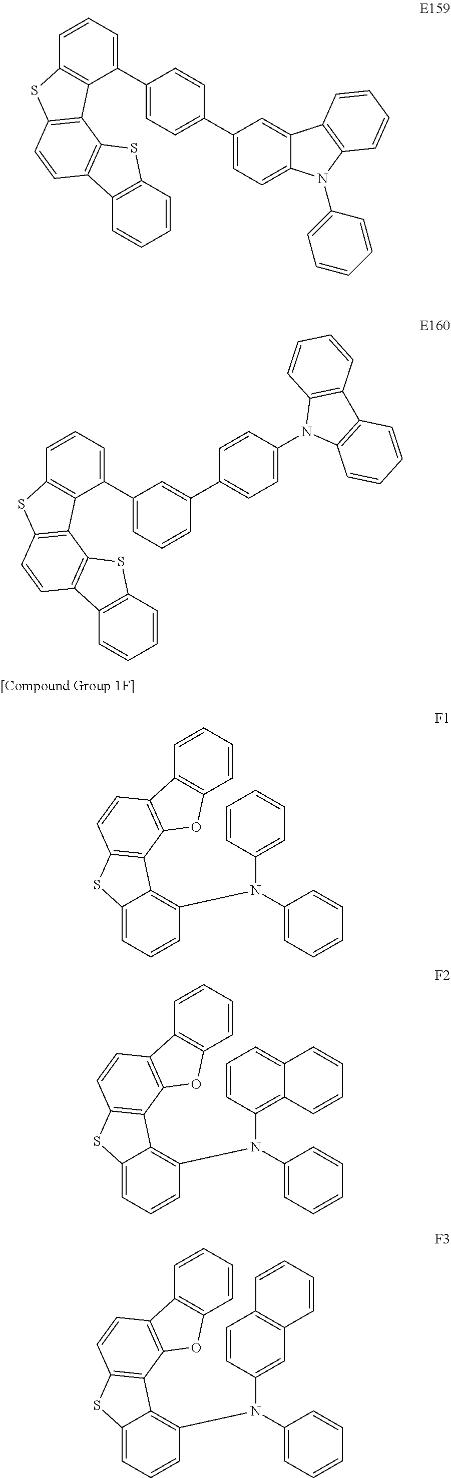 [SMILES: c1ccc(-n2c3ccccc3c3cc(-c4ccc(-c5cccc6sc7ccc8c9ccccc9sc8c7c56)cc4)ccc32)cc1 ; c1cc(-c2ccc(-n3c4ccccc4c4ccccc43)cc2)cc(-c2cccc3sc4ccc5c6ccccc6sc5c4c23)c1 ; c1ccc(N(c2ccccc2)c2cccc3sc4ccc5c6ccccc6oc5c4c23)cc1 ; c1ccc(N(c2cccc3ccccc23)c2cccc3sc4ccc5c6ccccc6oc5c4c23)cc1 ; c1ccc(N(c2ccc3ccccc3c2)c2cccc3sc4ccc5c6ccccc6oc5c4c23)cc1]

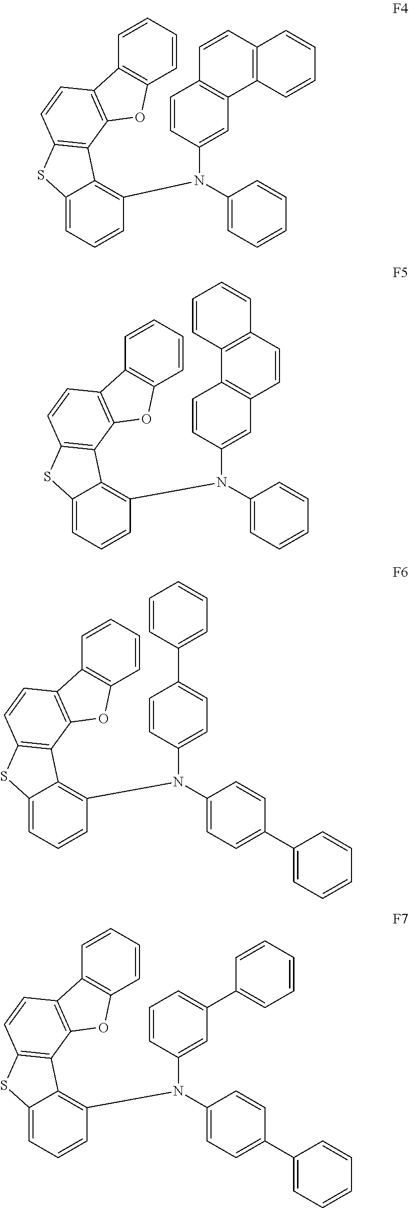 [SMILES: c1ccc(N(c2ccc3ccc4ccccc4c3c2)c2cccc3sc4ccc5c6ccccc6oc5c4c23)cc1 ; c1ccc(N(c2ccc3c(ccc4ccccc43)c2)c2cccc3sc4ccc5c6ccccc6oc5c4c23)cc1 ; c1ccc(-c2ccc(N(c3ccc(-c4ccccc4)cc3)c3cccc4sc5ccc6c7ccccc7oc6c5c34)cc2)cc1 ; c1ccc(-c2ccc(N(c3cccc(-c4ccccc4)c3)c3cccc4sc5ccc6c7ccccc7oc6c5c34)cc2)cc1]

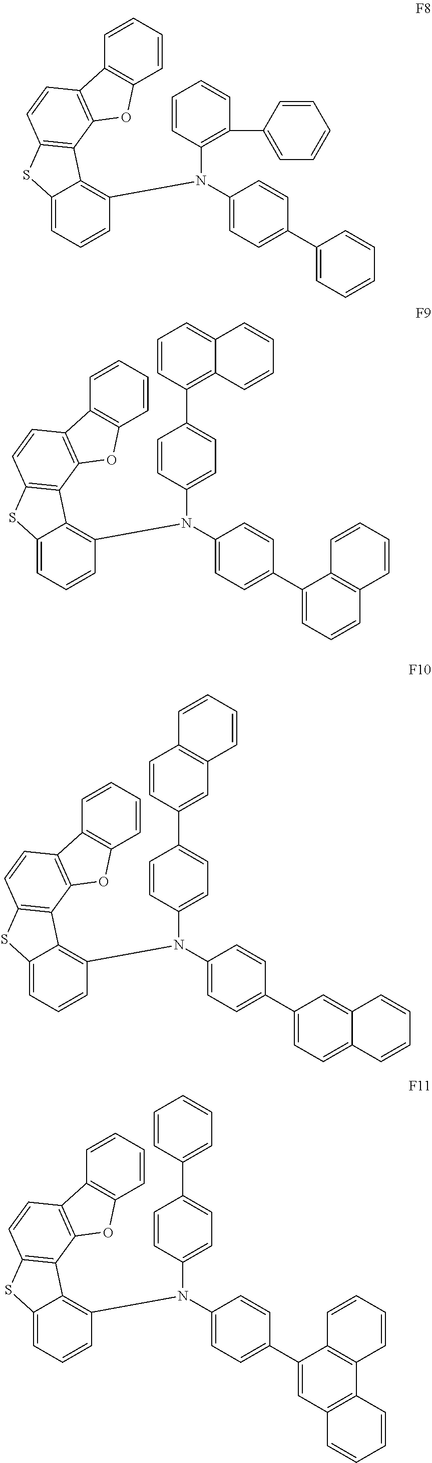 [SMILES: c1ccc(-c2ccc(N(c3ccccc3-c3ccccc3)c3cccc4sc5ccc6c7ccccc7oc6c5c34)cc2)cc1 ; c1ccc2c(-c3ccc(N(c4ccc(-c5cccc6ccccc56)cc4)c4cccc5sc6ccc7c8ccccc8oc7c6c45)cc3)cccc2c1 ; c1ccc2cc(-c3ccc(N(c4ccc(-c5ccc6ccccc6c5)cc4)c4cccc5sc6ccc7c8ccccc8oc7c6c45)cc3)ccc2c1 ; c1ccc(-c2ccc(N(c3ccc(-c4cc5ccccc5c5ccccc45)cc3)c3cccc4sc5ccc6c7ccccc7oc6c5c34)cc2)cc1]

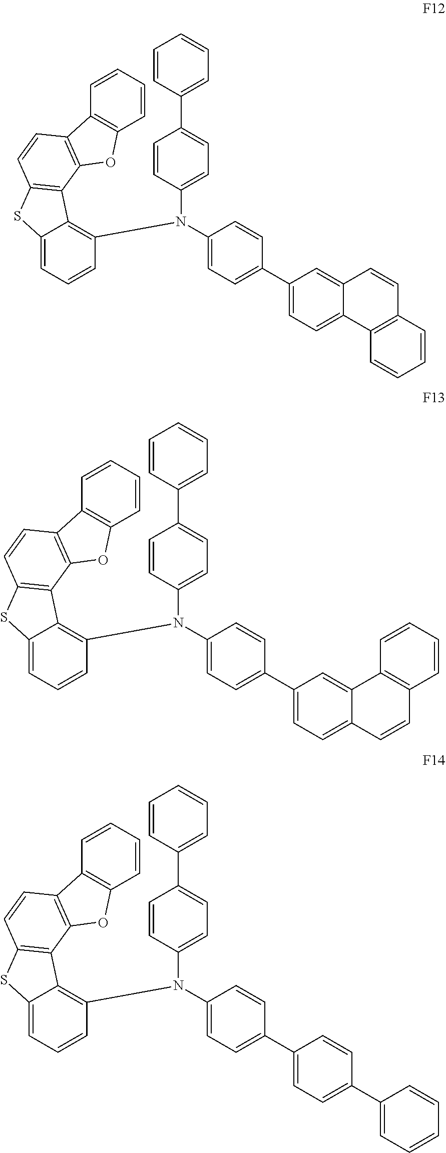 [SMILES: c1ccc(-c2ccc(N(c3ccc(-c4ccc5c(ccc6ccccc65)c4)cc3)c3cccc4sc5ccc6c7ccccc7oc6c5c34)cc2)cc1 ; c1ccc(-c2ccc(N(c3ccc(-c4ccc5ccc6ccccc6c5c4)cc3)c3cccc4sc5ccc6c7ccccc7oc6c5c34)cc2)cc1 ; c1ccc(-c2ccc(-c3ccc(N(c4ccc(-c5ccccc5)cc4)c4cccc5sc6ccc7c8ccccc8oc7c6c45)cc3)cc2)cc1]

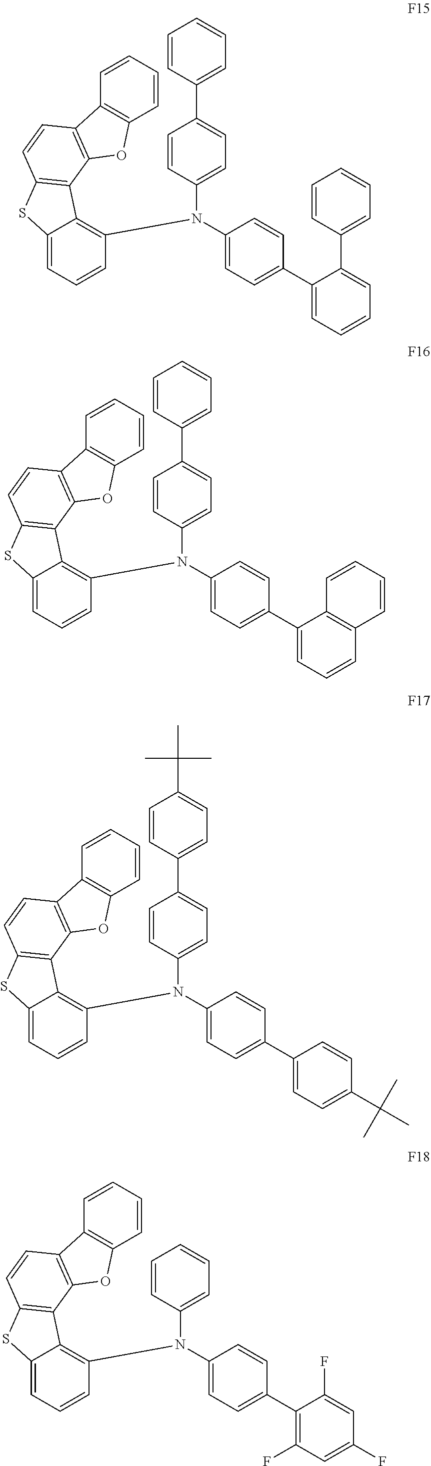 [SMILES: c1ccc(-c2ccc(N(c3ccc(-c4ccccc4-c4ccccc4)cc3)c3cccc4sc5ccc6c7ccccc7oc6c5c34)cc2)cc1 ; c1ccc(-c2ccc(N(c3ccc(-c4cccc5ccccc45)cc3)c3cccc4sc5ccc6c7ccccc7oc6c5c34)cc2)cc1 ; CC(C)(C)c1ccc(-c2ccc(N(c3ccc(-c4ccc(C(C)(C)C)cc4)cc3)c3cccc4sc5ccc6c7ccccc7oc6c5c34)cc2)cc1 ; Fc1cc(F)c(-c2ccc(N(c3ccccc3)c3cccc4sc5ccc6c7ccccc7oc6c5c34)cc2)c(F)c1]

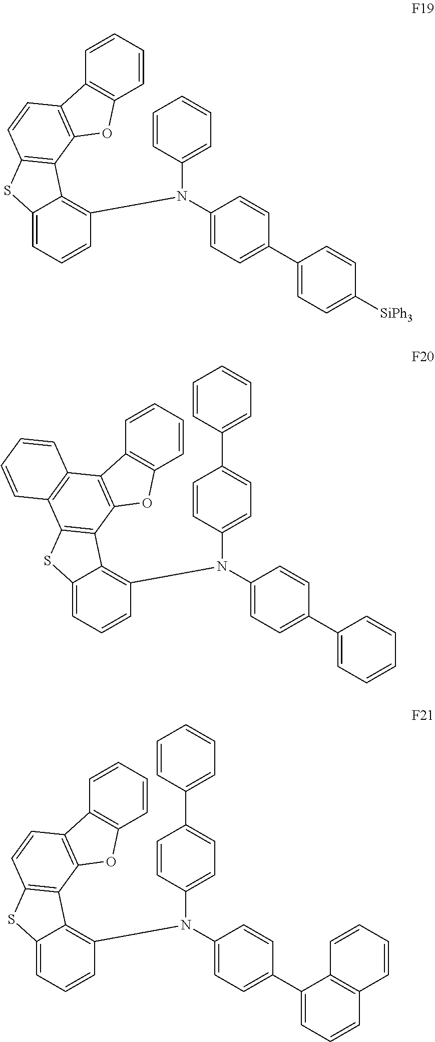

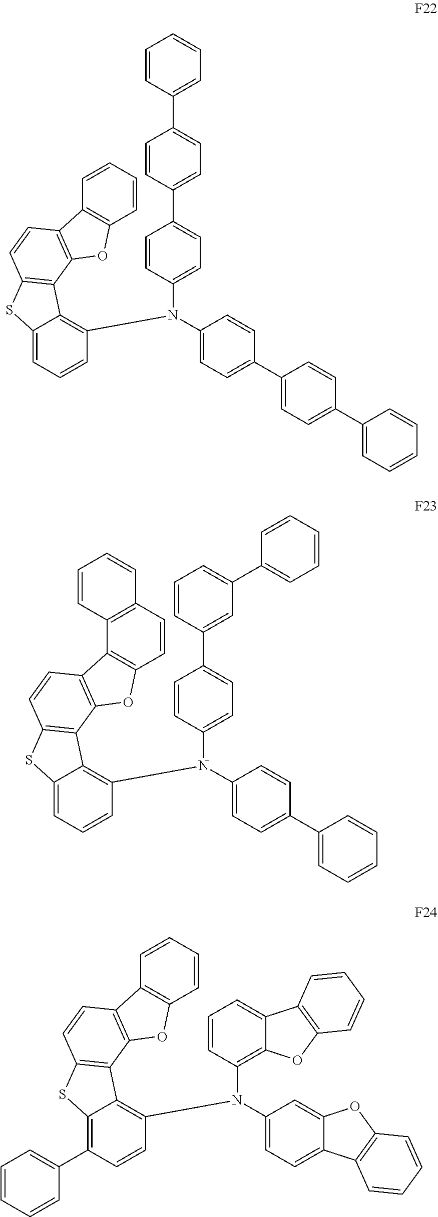 [SMILES: c1ccc(-c2ccc(-c3ccc(N(c4ccc(-c5ccc(-c6ccccc6)cc5)cc4)c4cccc5sc6ccc7c8ccccc8oc7c6c45)cc3)cc2)cc1 ; c1ccc(-c2ccc(N(c3ccc(-c4cccc(-c5ccccc5)c4)cc3)c3cccc4sc5ccc6c(oc7ccc8ccccc8c76)c5c34)cc2)cc1 ; c1ccc(-c2ccc(N(c3ccc4c(c3)oc3ccccc34)c3cccc4c3oc3ccccc34)c3c2sc2ccc4c5ccccc5oc4c23)cc1]

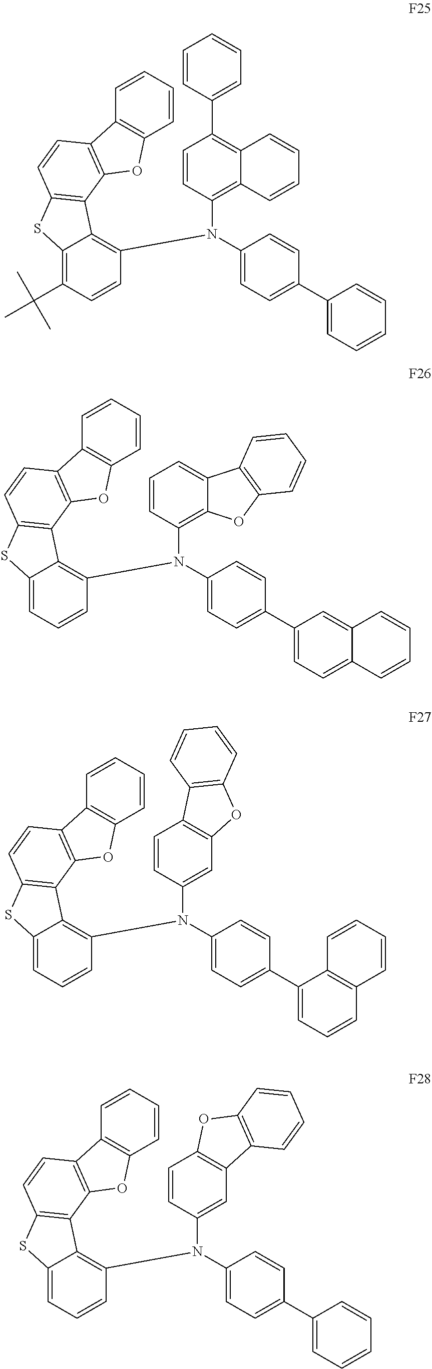 [SMILES: CC(C)(C)c1ccc(N(c2ccc(-c3ccccc3)cc2)c2ccc(-c3ccccc3)c3ccccc23)c2c1sc1ccc3c4ccccc4oc3c12 ; c1ccc2cc(-c3ccc(N(c4cccc5c4oc4ccccc45)c4cccc5sc6ccc7c8ccccc8oc7c6c45)cc3)ccc2c1 ; c1ccc2c(-c3ccc(N(c4ccc5c(c4)oc4ccccc45)c4cccc5sc6ccc7c8ccccc8oc7c6c45)cc3)cccc2c1 ; c1ccc(-c2ccc(N(c3ccc4oc5ccccc5c4c3)c3cccc4sc5ccc6c7ccccc7oc6c5c34)cc2)cc1]

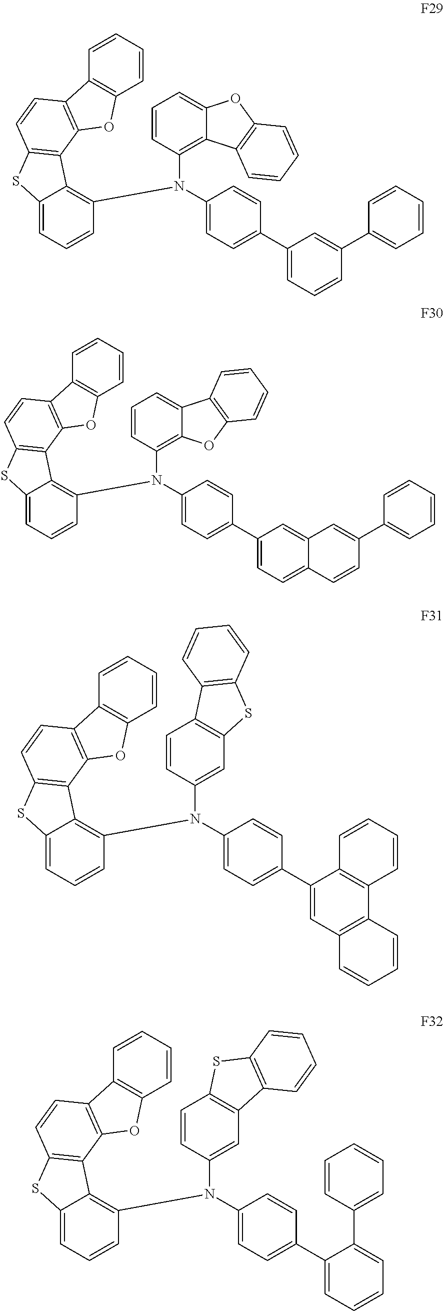 [SMILES: c1ccc(-c2cccc(-c3ccc(N(c4cccc5oc6ccccc6c45)c4cccc5sc6ccc7c8ccccc8oc7c6c45)cc3)c2)cc1 ; c1ccc(-c2ccc3ccc(-c4ccc(N(c5cccc6c5oc5ccccc56)c5cccc6sc7ccc8c9ccccc9oc8c7c56)cc4)cc3c2)cc1 ; c1ccc2c(c1)cc(-c1ccc(N(c3ccc4c(c3)sc3ccccc34)c3cccc4sc5ccc6c7ccccc7oc6c5c34)cc1)c1ccccc12 ; c1ccc(-c2ccccc2-c2ccc(N(c3ccc4sc5ccccc5c4c3)c3cccc4sc5ccc6c7ccccc7oc6c5c34)cc2)cc1]

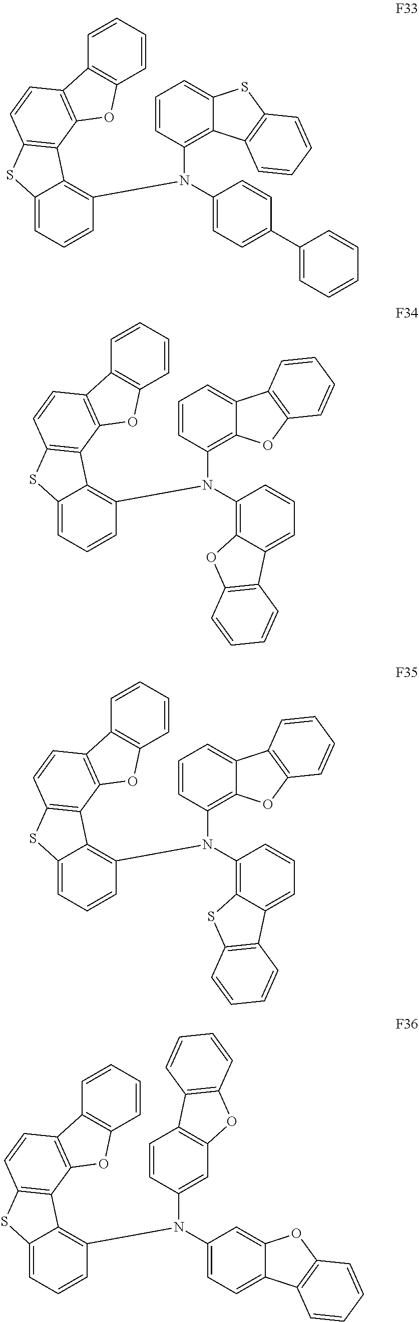

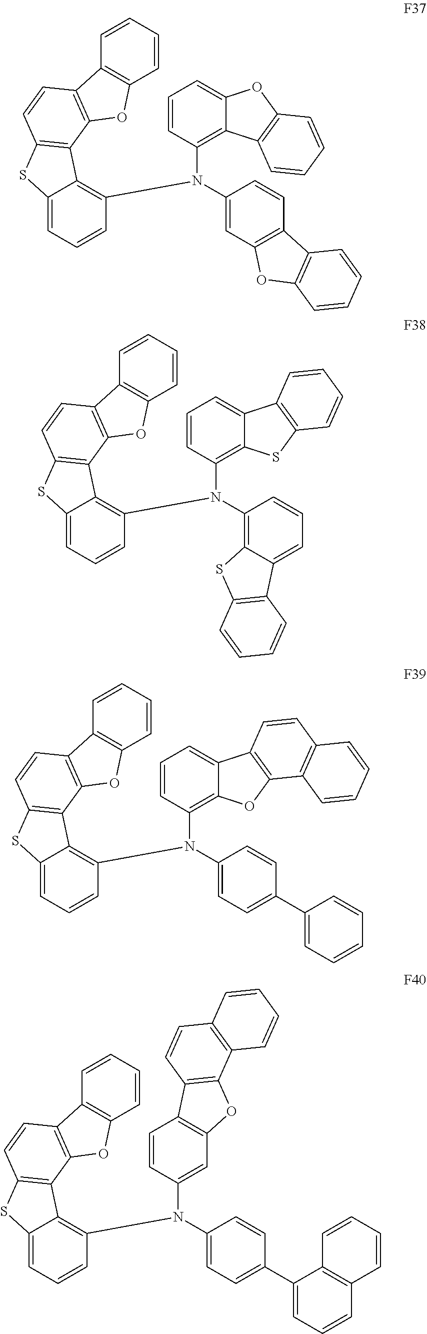 [SMILES: c1ccc2c(c1)oc1cc(N(c3cccc4oc5ccccc5c34)c3cccc4sc5ccc6c7ccccc7oc6c5c34)ccc12 ; c1ccc2c(c1)oc1c2ccc2sc3cccc(N(c4cccc5c4sc4ccccc45)c4cccc5c4sc4ccccc45)c3c21 ; c1ccc(-c2ccc(N(c3cccc4c3oc3c5ccccc5ccc43)c3cccc4sc5ccc6c7ccccc7oc6c5c34)cc2)cc1 ; c1ccc2c(-c3ccc(N(c4ccc5c(c4)oc4c6ccccc6ccc54)c4cccc5sc6ccc7c8ccccc8oc7c6c45)cc3)cccc2c1]

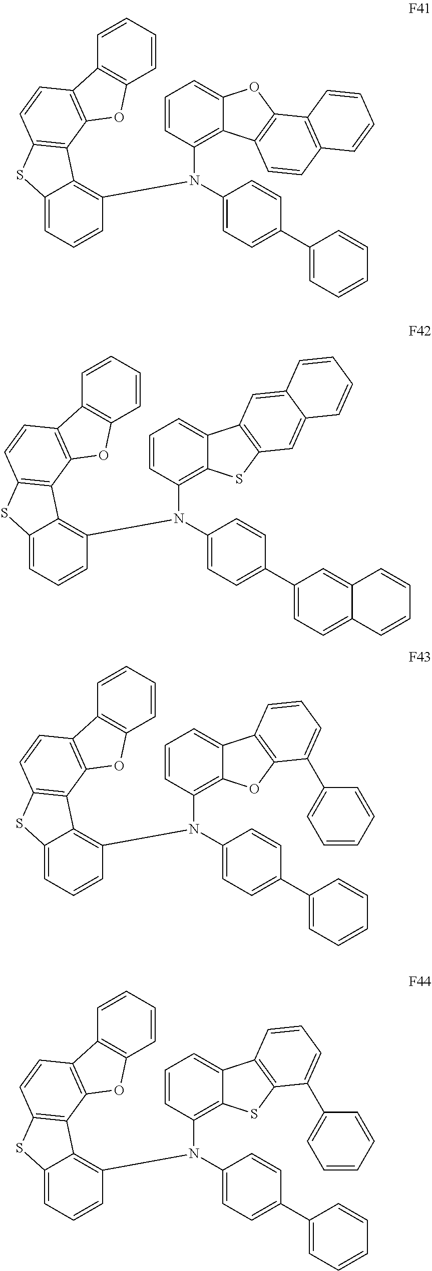 [SMILES: c1ccc(-c2ccc(N(c3cccc4oc5c6ccccc6ccc5c34)c3cccc4sc5ccc6c7ccccc7oc6c5c34)cc2)cc1 ; c1ccc2cc(-c3ccc(N(c4cccc5c4sc4cc6ccccc6cc45)c4cccc5sc6ccc7c8ccccc8oc7c6c45)cc3)ccc2c1 ; c1ccc(-c2ccc(N(c3cccc4c3oc3c(-c5ccccc5)cccc34)c3cccc4sc5ccc6c7ccccc7oc6c5c34)cc2)cc1 ; c1ccc(-c2ccc(N(c3cccc4c3sc3c(-c5ccccc5)cccc34)c3cccc4sc5ccc6c7ccccc7oc6c5c34)cc2)cc1]

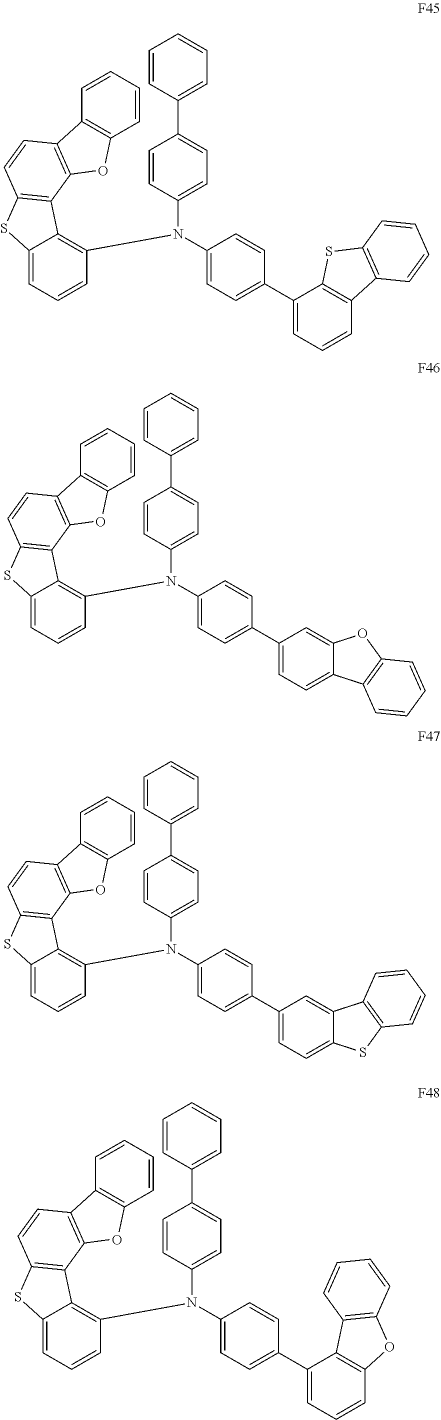 [SMILES: c1ccc(-c2ccc(N(c3ccc(-c4cccc5c4sc4ccccc45)cc3)c3cccc4sc5ccc6c7ccccc7oc6c5c34)cc2)cc1 ; c1ccc(-c2ccc(N(c3ccc(-c4ccc5c(c4)oc4ccccc45)cc3)c3cccc4sc5ccc6c7ccccc7oc6c5c34)cc2)cc1 ; c1ccc(-c2ccc(N(c3ccc(-c4ccc5sc6ccccc6c5c4)cc3)c3cccc4sc5ccc6c7ccccc7oc6c5c34)cc2)cc1 ; c1ccc(-c2ccc(N(c3ccc(-c4cccc5oc6ccccc6c45)cc3)c3cccc4sc5ccc6c7ccccc7oc6c5c34)cc2)cc1]

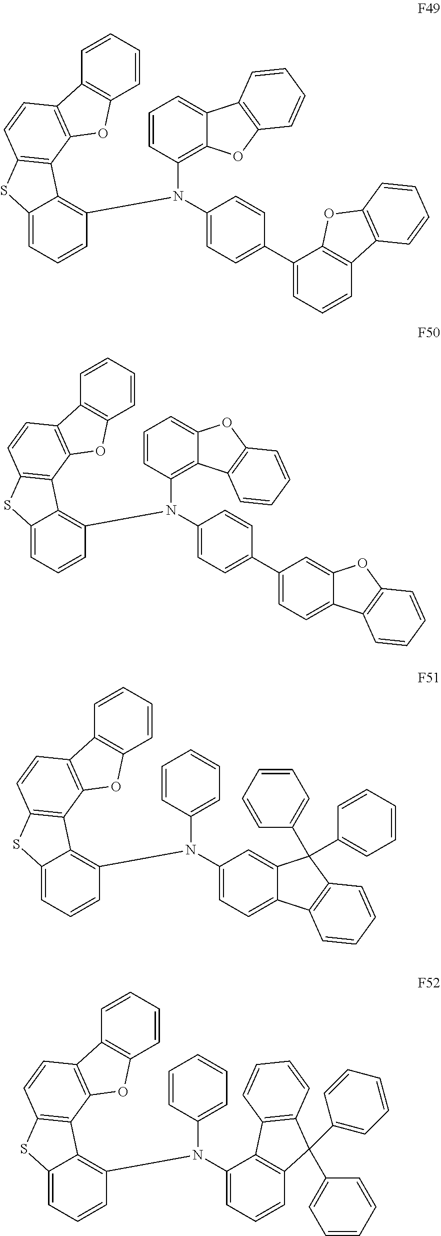 [SMILES: c1ccc2c(c1)oc1c(-c3ccc(N(c4cccc5c4oc4ccccc45)c4cccc5sc6ccc7c8ccccc8oc7c6c45)cc3)cccc12 ; c1ccc2c(c1)oc1cc(-c3ccc(N(c4cccc5oc6ccccc6c45)c4cccc5sc6ccc7c8ccccc8oc7c6c45)cc3)ccc12 ; c1ccc(N(c2ccc3c(c2)C(c2ccccc2)(c2ccccc2)c2ccccc2-3)c2cccc3sc4ccc5c6ccccc6oc5c4c23)cc1 ; c1ccc(N(c2cccc3c2-c2ccccc2C3(c2ccccc2)c2ccccc2)c2cccc3sc4ccc5c6ccccc6oc5c4c23)cc1]

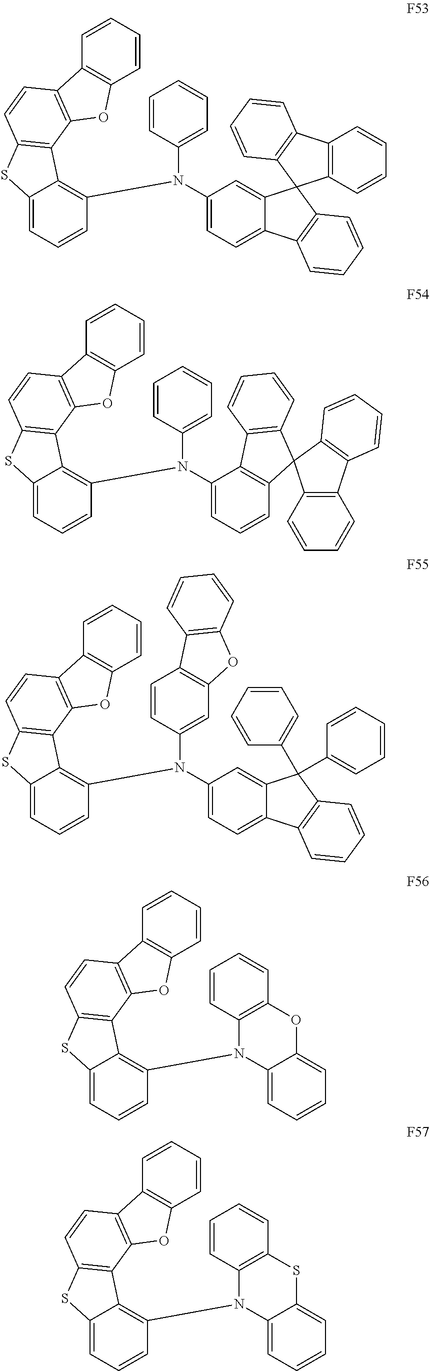

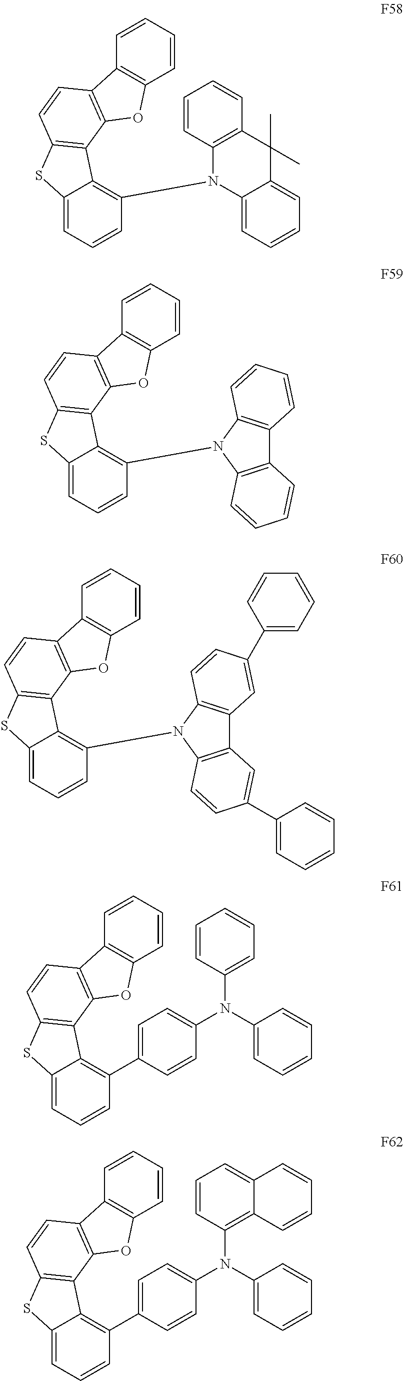

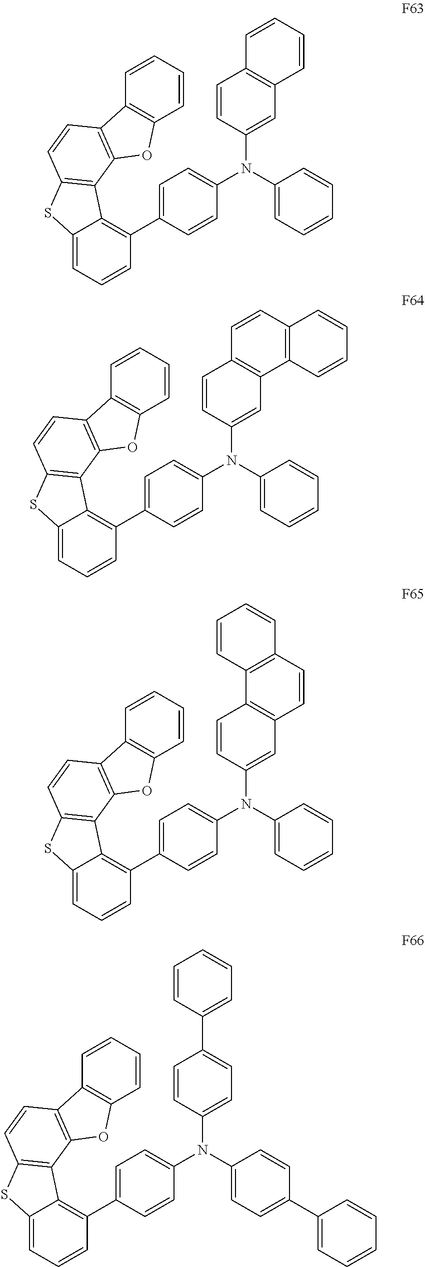 [SMILES: c1ccc(N(c2ccc(-c3cccc4sc5ccc6c7ccccc7oc6c5c34)cc2)c2ccc3ccccc3c2)cc1 ; c1ccc(N(c2ccc(-c3cccc4sc5ccc6c7ccccc7oc6c5c34)cc2)c2ccc3ccc4ccccc4c3c2)cc1 ; c1ccc(N(c2ccc(-c3cccc4sc5ccc6c7ccccc7oc6c5c34)cc2)c2ccc3c(ccc4ccccc43)c2)cc1 ; c1ccc(-c2ccc(N(c3ccc(-c4ccccc4)cc3)c3ccc(-c4cccc5sc6ccc7c8ccccc8oc7c6c45)cc3)cc2)cc1]

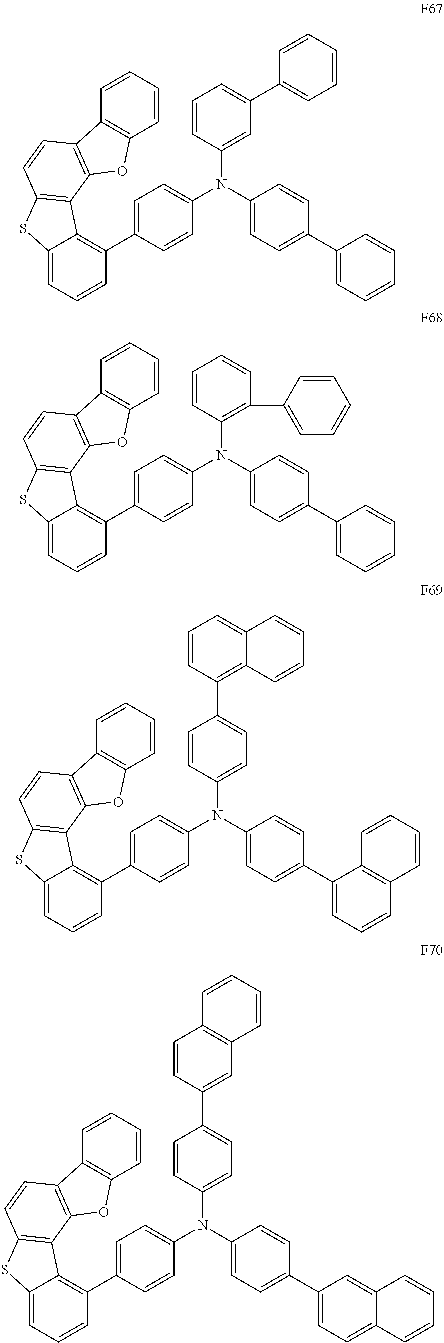 [SMILES: c1ccc(-c2ccc(N(c3ccc(-c4cccc5sc6ccc7c8ccccc8oc7c6c45)cc3)c3cccc(-c4ccccc4)c3)cc2)cc1 ; c1ccc(-c2ccc(N(c3ccc(-c4cccc5sc6ccc7c8ccccc8oc7c6c45)cc3)c3ccccc3-c3ccccc3)cc2)cc1 ; c1ccc2c(-c3ccc(N(c4ccc(-c5cccc6ccccc56)cc4)c4ccc(-c5cccc6sc7ccc8c9ccccc9oc8c7c56)cc4)cc3)cccc2c1 ; c1ccc2cc(-c3ccc(N(c4ccc(-c5ccc6ccccc6c5)cc4)c4ccc(-c5cccc6sc7ccc8c9ccccc9oc8c7c56)cc4)cc3)ccc2c1]

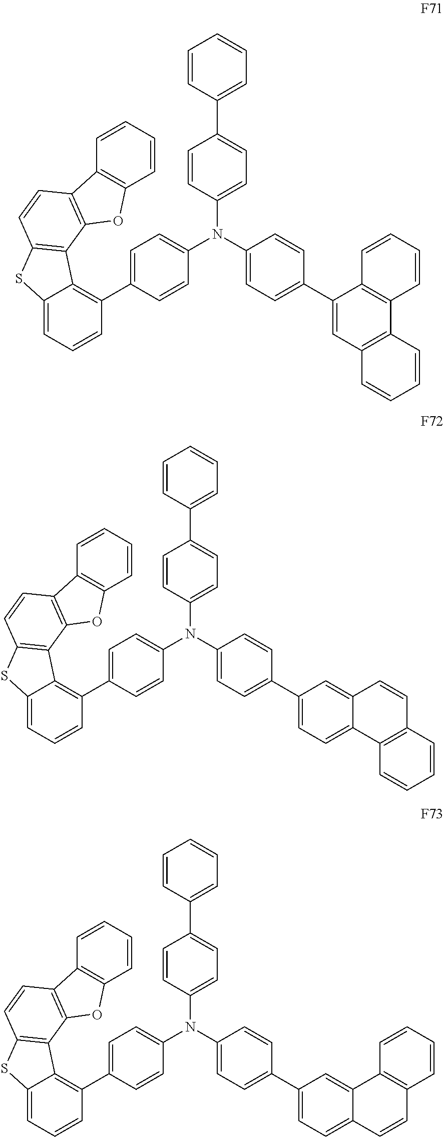 [SMILES: c1ccc(-c2ccc(N(c3ccc(-c4cc5ccccc5c5ccccc45)cc3)c3ccc(-c4cccc5sc6ccc7c8ccccc8oc7c6c45)cc3)cc2)cc1 ; c1ccc(-c2ccc(N(c3ccc(-c4ccc5c(ccc6ccccc65)c4)cc3)c3ccc(-c4cccc5sc6ccc7c8ccccc8oc7c6c45)cc3)cc2)cc1 ; c1ccc(-c2ccc(N(c3ccc(-c4ccc5ccc6ccccc6c5c4)cc3)c3ccc(-c4cccc5sc6ccc7c8ccccc8oc7c6c45)cc3)cc2)cc1]

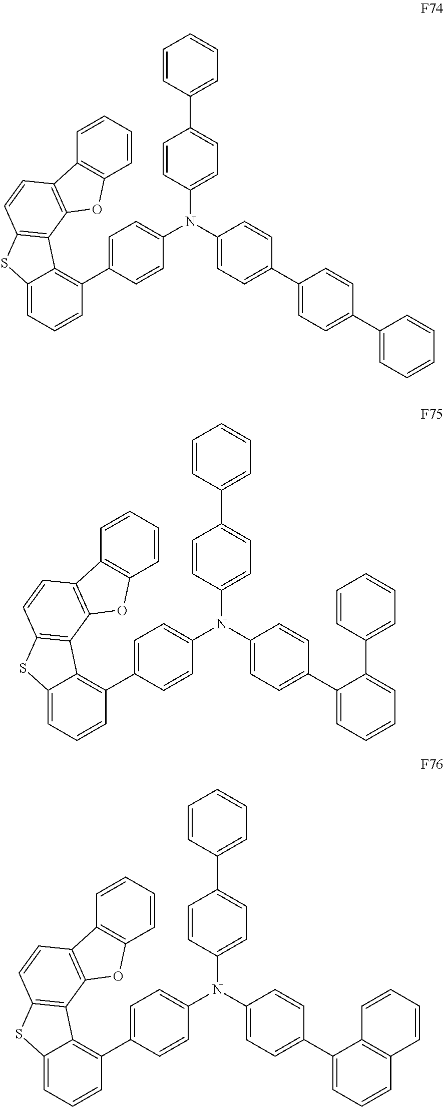 [SMILES: c1ccc(-c2ccc(-c3ccc(N(c4ccc(-c5ccccc5)cc4)c4ccc(-c5cccc6sc7ccc8c9ccccc9oc8c7c56)cc4)cc3)cc2)cc1 ; c1ccc(-c2ccc(N(c3ccc(-c4ccccc4-c4ccccc4)cc3)c3ccc(-c4cccc5sc6ccc7c8ccccc8oc7c6c45)cc3)cc2)cc1 ; c1ccc(-c2ccc(N(c3ccc(-c4cccc5ccccc45)cc3)c3ccc(-c4cccc5sc6ccc7c8ccccc8oc7c6c45)cc3)cc2)cc1]

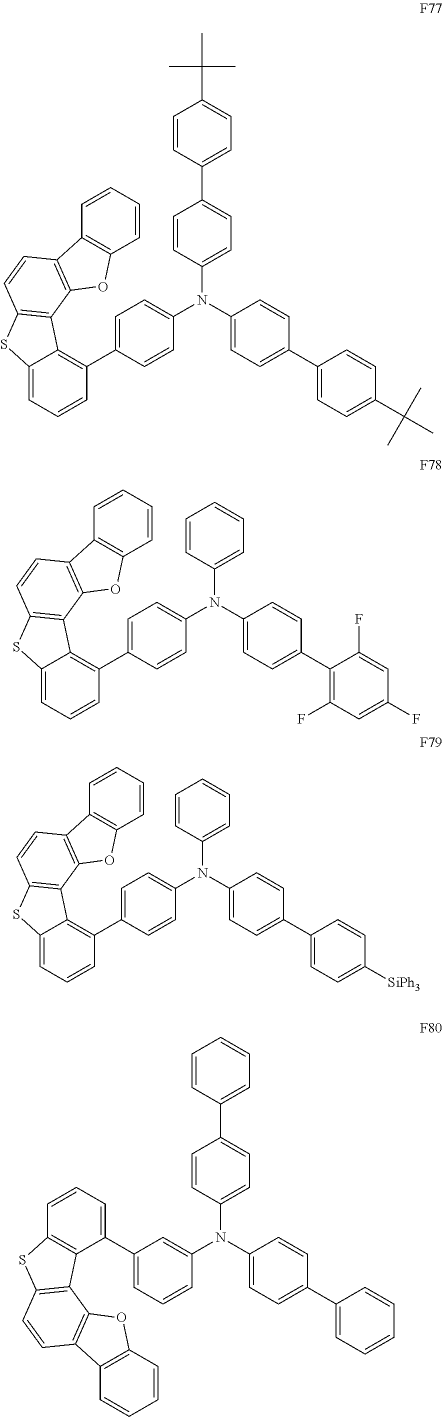

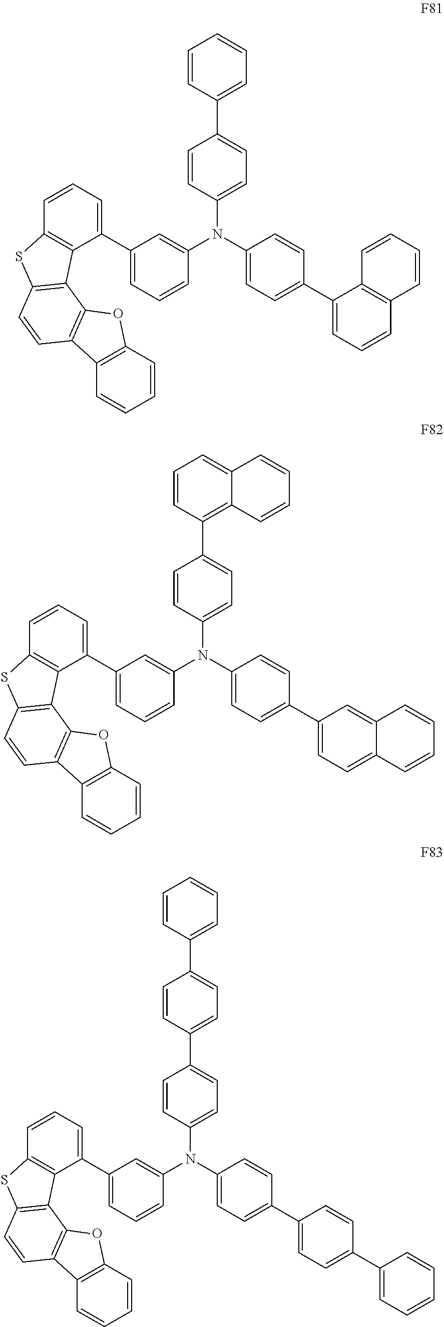 [SMILES: c1ccc(-c2ccc(N(c3ccc(-c4cccc5ccccc45)cc3)c3cccc(-c4cccc5sc6ccc7c8ccccc8oc7c6c45)c3)cc2)cc1 ; c1cc(-c2cccc3sc4ccc5c6ccccc6oc5c4c23)cc(N(c2ccc(-c3ccc4ccccc4c3)cc2)c2ccc(-c3cccc4ccccc34)cc2)c1 ; c1ccc(-c2ccc(-c3ccc(N(c4ccc(-c5ccc(-c6ccccc6)cc5)cc4)c4cccc(-c5cccc6sc7ccc8c9ccccc9oc8c7c56)c4)cc3)cc2)cc1]

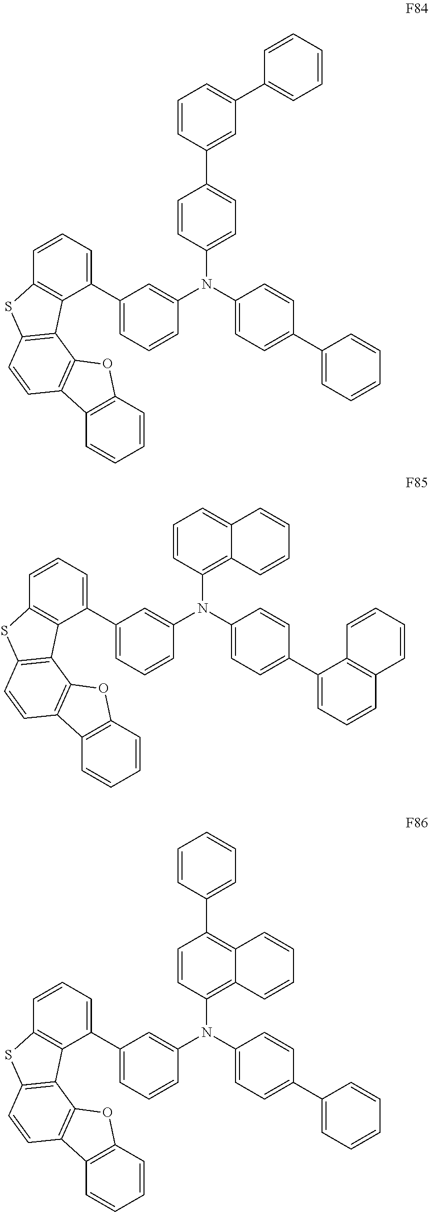 [SMILES: c1ccc(-c2ccc(N(c3ccc(-c4cccc(-c5ccccc5)c4)cc3)c3cccc(-c4cccc5sc6ccc7c8ccccc8oc7c6c45)c3)cc2)cc1 ; c1cc(-c2cccc3sc4ccc5c6ccccc6oc5c4c23)cc(N(c2ccc(-c3cccc4ccccc34)cc2)c2cccc3ccccc23)c1 ; c1ccc(-c2ccc(N(c3cccc(-c4cccc5sc6ccc7c8ccccc8oc7c6c45)c3)c3ccc(-c4ccccc4)c4ccccc34)cc2)cc1]

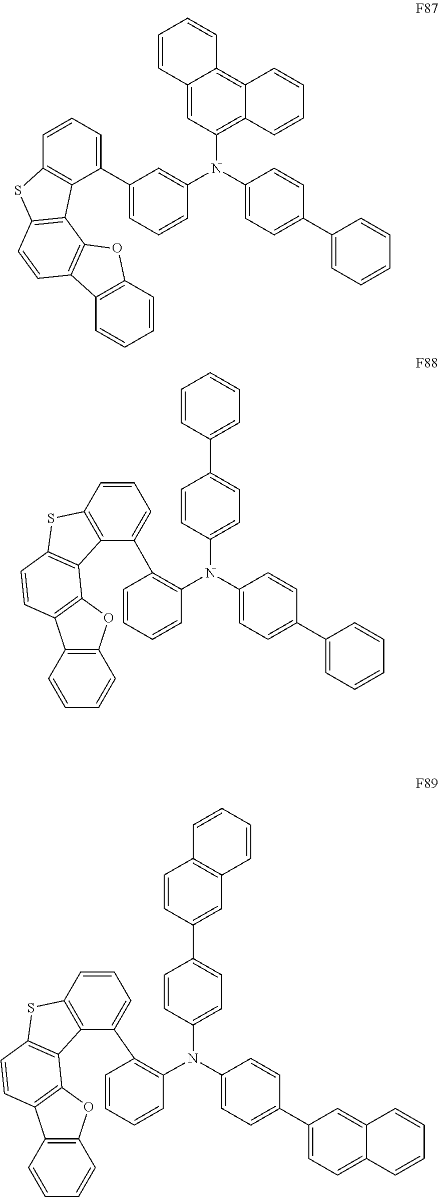 [SMILES: c1ccc(-c2ccc(N(c3cccc(-c4cccc5sc6ccc7c8ccccc8oc7c6c45)c3)c3cc4ccccc4c4ccccc34)cc2)cc1 ; c1ccc(-c2ccc(N(c3ccc(-c4ccccc4)cc3)c3ccccc3-c3cccc4sc5ccc6c7ccccc7oc6c5c34)cc2)cc1 ; c1ccc(N(c2ccc(-c3ccc4ccccc4c3)cc2)c2ccc(-c3ccc4ccccc4c3)cc2)c(-c2cccc3sc4ccc5c6ccccc6oc5c4c23)c1]

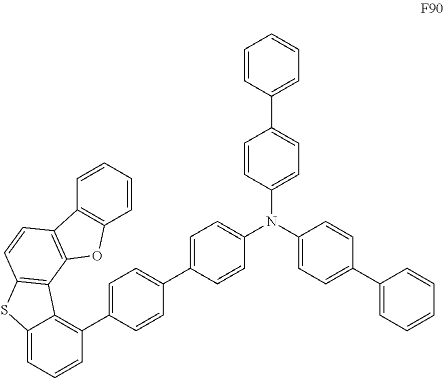 [SMILES: c1ccc(-c2ccc(N(c3ccc(-c4ccccc4)cc3)c3ccc(-c4ccc(-c5cccc6sc7ccc8c9ccccc9oc8c7c56)cc4)cc3)cc2)cc1]

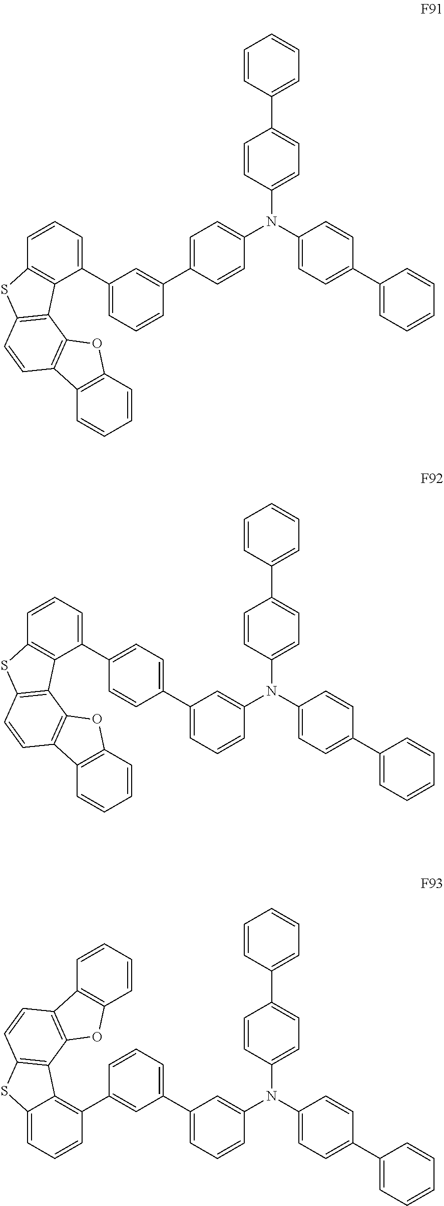 [SMILES: c1ccc(-c2ccc(N(c3ccc(-c4ccccc4)cc3)c3ccc(-c4cccc(-c5cccc6sc7ccc8c9ccccc9oc8c7c56)c4)cc3)cc2)cc1 ; c1ccc(-c2ccc(N(c3ccc(-c4ccccc4)cc3)c3cccc(-c4ccc(-c5cccc6sc7ccc8c9ccccc9oc8c7c56)cc4)c3)cc2)cc1 ; c1ccc(-c2ccc(N(c3ccc(-c4ccccc4)cc3)c3cccc(-c4cccc(-c5cccc6sc7ccc8c9ccccc9oc8c7c56)c4)c3)cc2)cc1]

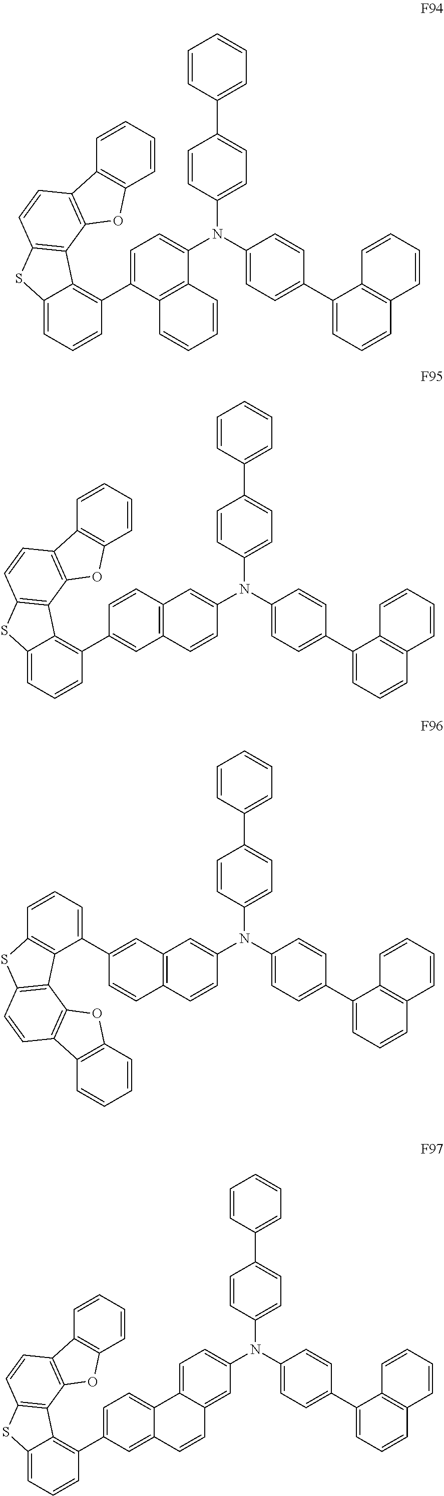 [SMILES: c1ccc(-c2ccc(N(c3ccc(-c4cccc5ccccc45)cc3)c3ccc(-c4cccc5sc6ccc7c8ccccc8oc7c6c45)c4ccccc34)cc2)cc1 ; c1ccc(-c2ccc(N(c3ccc(-c4cccc5ccccc45)cc3)c3ccc4cc(-c5cccc6sc7ccc8c9ccccc9oc8c7c56)ccc4c3)cc2)cc1 ; c1ccc(-c2ccc(N(c3ccc(-c4cccc5ccccc45)cc3)c3ccc4ccc(-c5cccc6sc7ccc8c9ccccc9oc8c7c56)cc4c3)cc2)cc1 ; c1ccc(-c2ccc(N(c3ccc(-c4cccc5ccccc45)cc3)c3ccc4c(ccc5cc(-c6cccc7sc8ccc9c%10ccccc%10oc9c8c67)ccc54)c3)cc2)cc1]

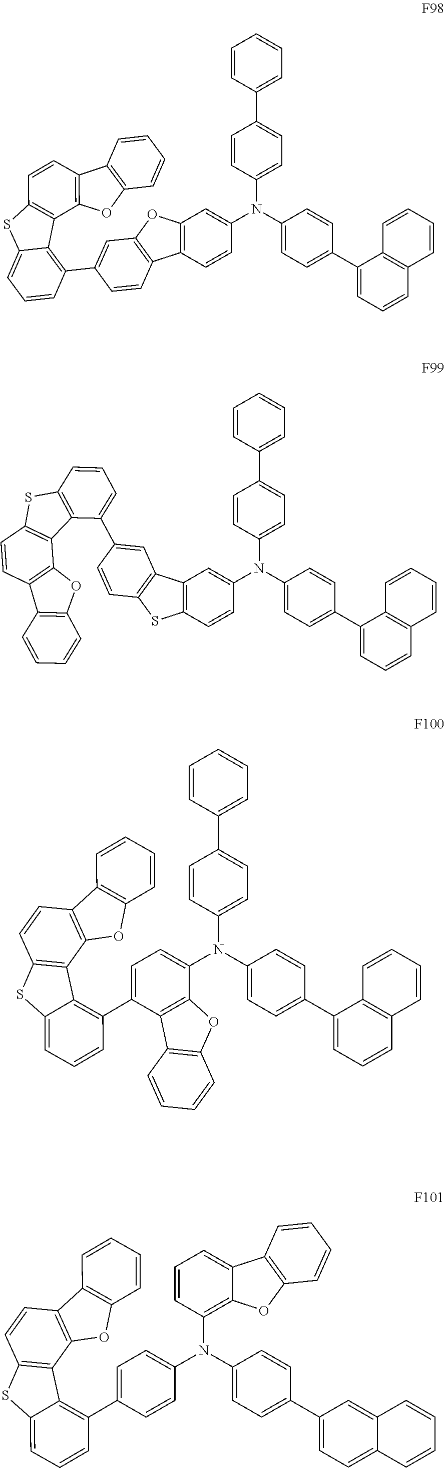 [SMILES: c1ccc(-c2ccc(N(c3ccc(-c4cccc5ccccc45)cc3)c3ccc4c(c3)oc3cc(-c5cccc6sc7ccc8c9ccccc9oc8c7c56)ccc34)cc2)cc1 ; c1ccc(-c2ccc(N(c3ccc(-c4cccc5ccccc45)cc3)c3ccc4sc5ccc(-c6cccc7sc8ccc9c%10ccccc%10oc9c8c67)cc5c4c3)cc2)cc1 ; c1ccc(-c2ccc(N(c3ccc(-c4cccc5ccccc45)cc3)c3ccc(-c4cccc5sc6ccc7c8ccccc8oc7c6c45)c4c3oc3ccccc34)cc2)cc1 ; c1ccc2cc(-c3ccc(N(c4ccc(-c5cccc6sc7ccc8c9ccccc9oc8c7c56)cc4)c4cccc5c4oc4ccccc45)cc3)ccc2c1]

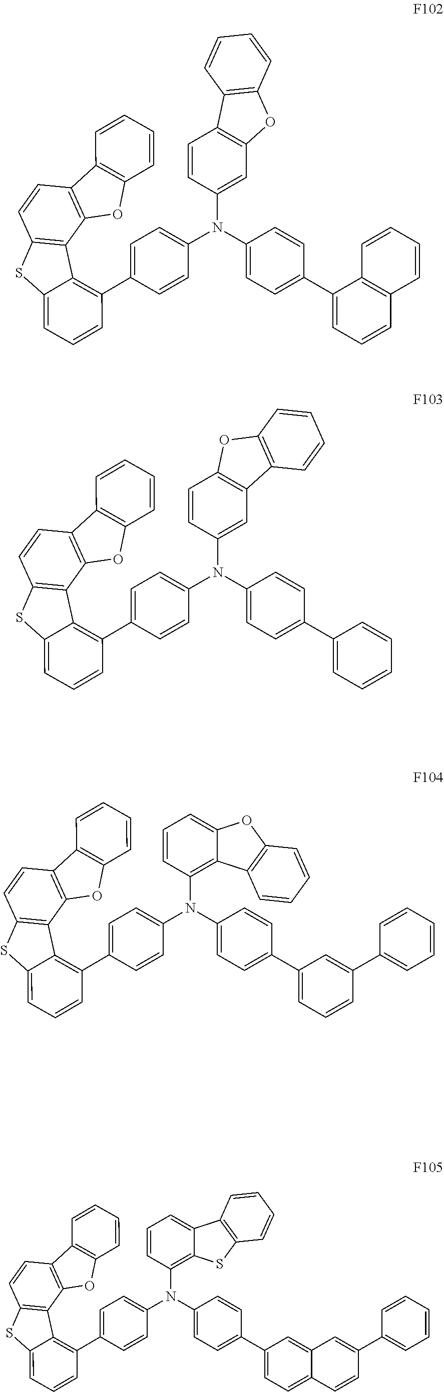 [SMILES: c1ccc2c(-c3ccc(N(c4ccc(-c5cccc6sc7ccc8c9ccccc9oc8c7c56)cc4)c4ccc5c(c4)oc4ccccc45)cc3)cccc2c1 ; c1ccc(-c2ccc(N(c3ccc(-c4cccc5sc6ccc7c8ccccc8oc7c6c45)cc3)c3ccc4oc5ccccc5c4c3)cc2)cc1 ; c1ccc(-c2cccc(-c3ccc(N(c4ccc(-c5cccc6sc7ccc8c9ccccc9oc8c7c56)cc4)c4cccc5oc6ccccc6c45)cc3)c2)cc1 ; c1ccc(-c2ccc3ccc(-c4ccc(N(c5ccc(-c6cccc7sc8ccc9c%10ccccc%10oc9c8c67)cc5)c5cccc6c5sc5ccccc56)cc4)cc3c2)cc1]

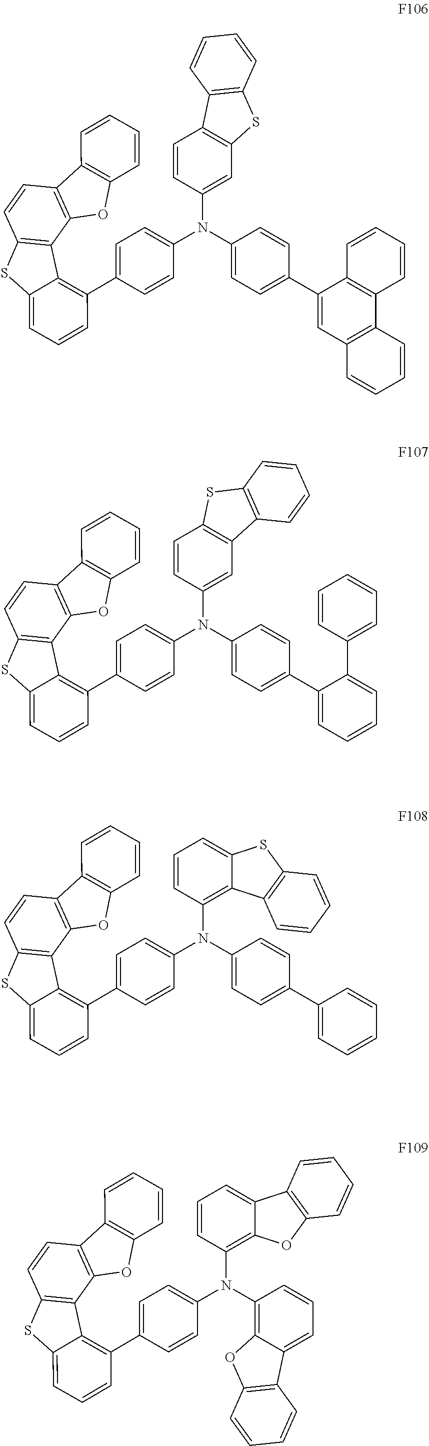 [SMILES: c1ccc2c(c1)cc(-c1ccc(N(c3ccc(-c4cccc5sc6ccc7c8ccccc8oc7c6c45)cc3)c3ccc4c(c3)sc3ccccc34)cc1)c1ccccc12 ; c1ccc(-c2ccccc2-c2ccc(N(c3ccc(-c4cccc5sc6ccc7c8ccccc8oc7c6c45)cc3)c3ccc4sc5ccccc5c4c3)cc2)cc1 ; c1ccc(-c2ccc(N(c3ccc(-c4cccc5sc6ccc7c8ccccc8oc7c6c45)cc3)c3cccc4sc5ccccc5c34)cc2)cc1 ; c1ccc2c(c1)oc1c(N(c3ccc(-c4cccc5sc6ccc7c8ccccc8oc7c6c45)cc3)c3cccc4c3oc3ccccc34)cccc12]

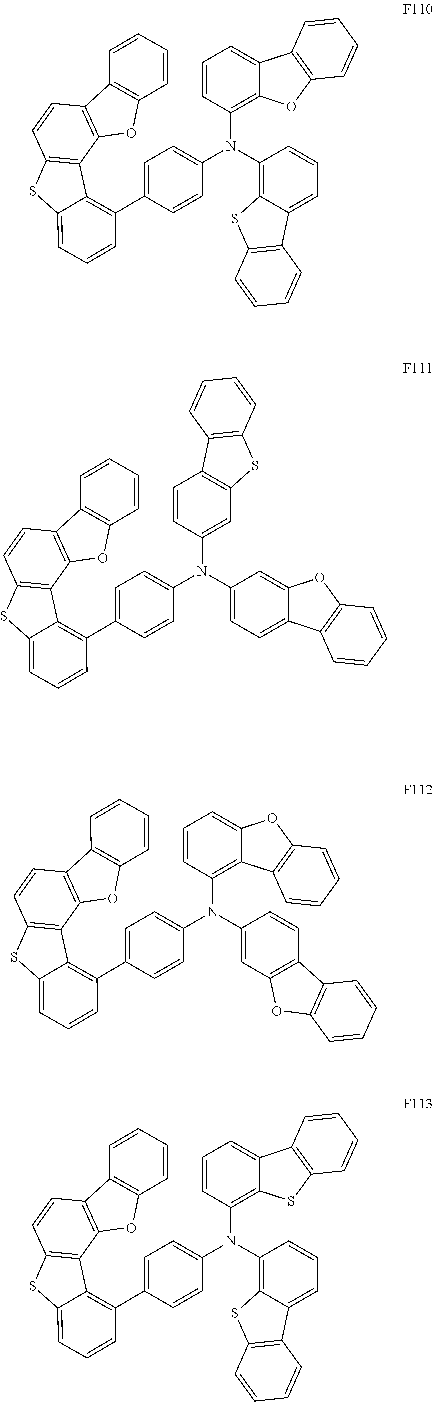 [SMILES: c1ccc2c(c1)oc1c(N(c3ccc(-c4cccc5sc6ccc7c8ccccc8oc7c6c45)cc3)c3cccc4c3sc3ccccc34)cccc12 ; c1ccc2c(c1)oc1cc(N(c3ccc(-c4cccc5sc6ccc7c8ccccc8oc7c6c45)cc3)c3ccc4c(c3)sc3ccccc34)ccc12 ; c1ccc2c(c1)oc1cc(N(c3ccc(-c4cccc5sc6ccc7c8ccccc8oc7c6c45)cc3)c3cccc4oc5ccccc5c34)ccc12 ; c1ccc2c(c1)oc1c2ccc2sc3cccc(-c4ccc(N(c5cccc6c5sc5ccccc56)c5cccc6c5sc5ccccc56)cc4)c3c21]

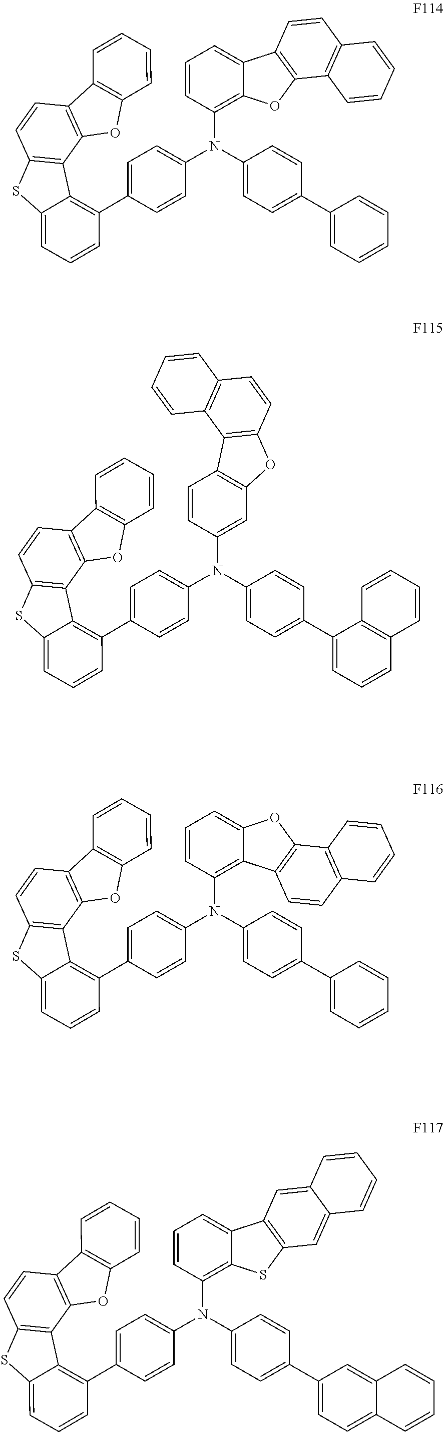 [SMILES: c1ccc(-c2ccc(N(c3ccc(-c4cccc5sc6ccc7c8ccccc8oc7c6c45)cc3)c3cccc4c3oc3c5ccccc5ccc43)cc2)cc1 ; c1ccc2c(-c3ccc(N(c4ccc(-c5cccc6sc7ccc8c9ccccc9oc8c7c56)cc4)c4ccc5c(c4)oc4ccc6ccccc6c45)cc3)cccc2c1 ; c1ccc(-c2ccc(N(c3ccc(-c4cccc5sc6ccc7c8ccccc8oc7c6c45)cc3)c3cccc4oc5c6ccccc6ccc5c34)cc2)cc1 ; c1ccc2cc(-c3ccc(N(c4ccc(-c5cccc6sc7ccc8c9ccccc9oc8c7c56)cc4)c4cccc5c4sc4cc6ccccc6cc45)cc3)ccc2c1]

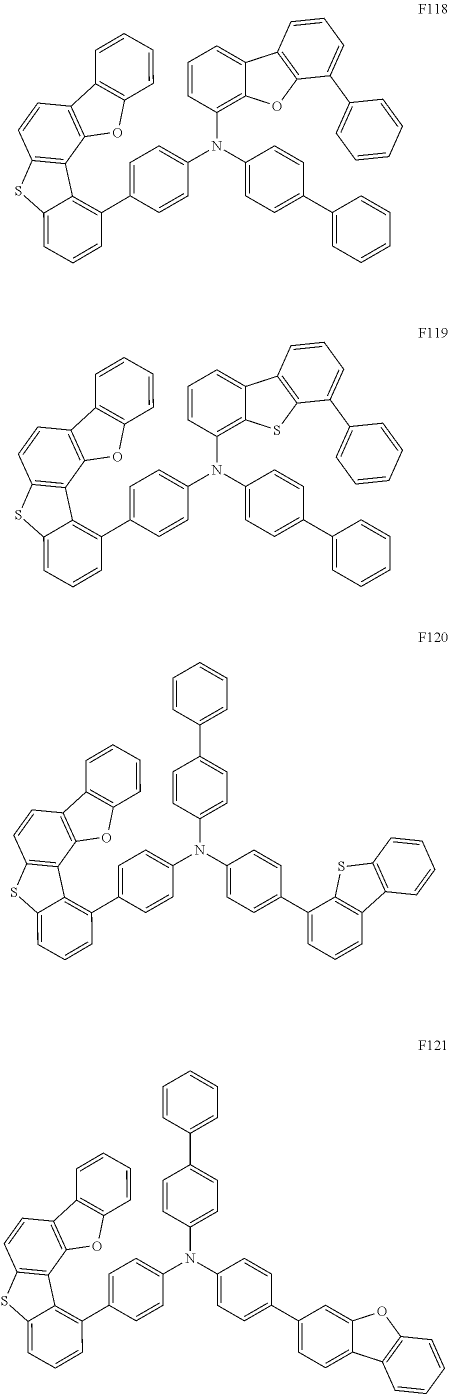 [SMILES: c1ccc(-c2ccc(N(c3ccc(-c4cccc5sc6ccc7c8ccccc8oc7c6c45)cc3)c3cccc4c3oc3c(-c5ccccc5)cccc34)cc2)cc1 ; c1ccc(-c2ccc(N(c3ccc(-c4cccc5sc6ccc7c8ccccc8oc7c6c45)cc3)c3cccc4c3sc3c(-c5ccccc5)cccc34)cc2)cc1 ; c1ccc(-c2ccc(N(c3ccc(-c4cccc5c4sc4ccccc45)cc3)c3ccc(-c4cccc5sc6ccc7c8ccccc8oc7c6c45)cc3)cc2)cc1 ; c1ccc(-c2ccc(N(c3ccc(-c4ccc5c(c4)oc4ccccc45)cc3)c3ccc(-c4cccc5sc6ccc7c8ccccc8oc7c6c45)cc3)cc2)cc1]

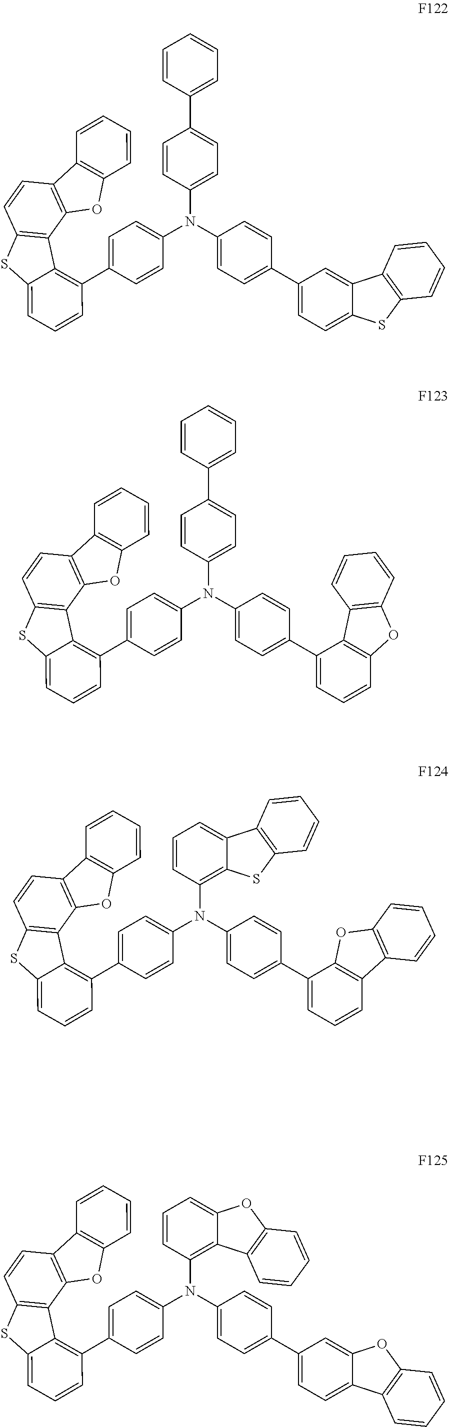 [SMILES: c1ccc(-c2ccc(N(c3ccc(-c4ccc5sc6ccccc6c5c4)cc3)c3ccc(-c4cccc5sc6ccc7c8ccccc8oc7c6c45)cc3)cc2)cc1 ; c1ccc(-c2ccc(N(c3ccc(-c4cccc5oc6ccccc6c45)cc3)c3ccc(-c4cccc5sc6ccc7c8ccccc8oc7c6c45)cc3)cc2)cc1 ; c1ccc2c(c1)oc1c(-c3ccc(N(c4ccc(-c5cccc6sc7ccc8c9ccccc9oc8c7c56)cc4)c4cccc5c4sc4ccccc45)cc3)cccc12 ; c1ccc2c(c1)oc1cc(-c3ccc(N(c4ccc(-c5cccc6sc7ccc8c9ccccc9oc8c7c56)cc4)c4cccc5oc6ccccc6c45)cc3)ccc12]

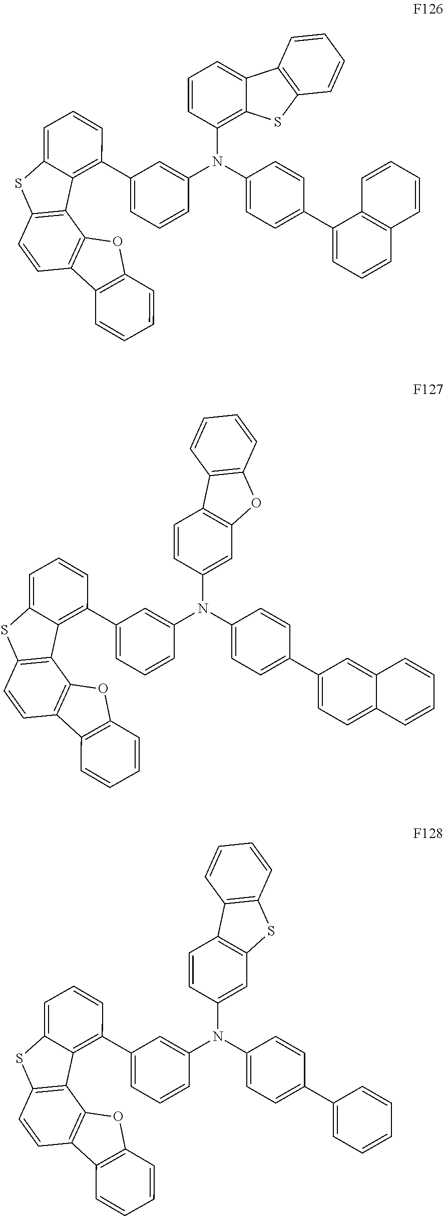 [SMILES: c1cc(-c2cccc3sc4ccc5c6ccccc6oc5c4c23)cc(N(c2ccc(-c3cccc4ccccc34)cc2)c2cccc3c2sc2ccccc23)c1 ; c1cc(-c2cccc3sc4ccc5c6ccccc6oc5c4c23)cc(N(c2ccc(-c3ccc4ccccc4c3)cc2)c2ccc3c(c2)oc2ccccc23)c1 ; c1ccc(-c2ccc(N(c3cccc(-c4cccc5sc6ccc7c8ccccc8oc7c6c45)c3)c3ccc4c(c3)sc3ccccc34)cc2)cc1]

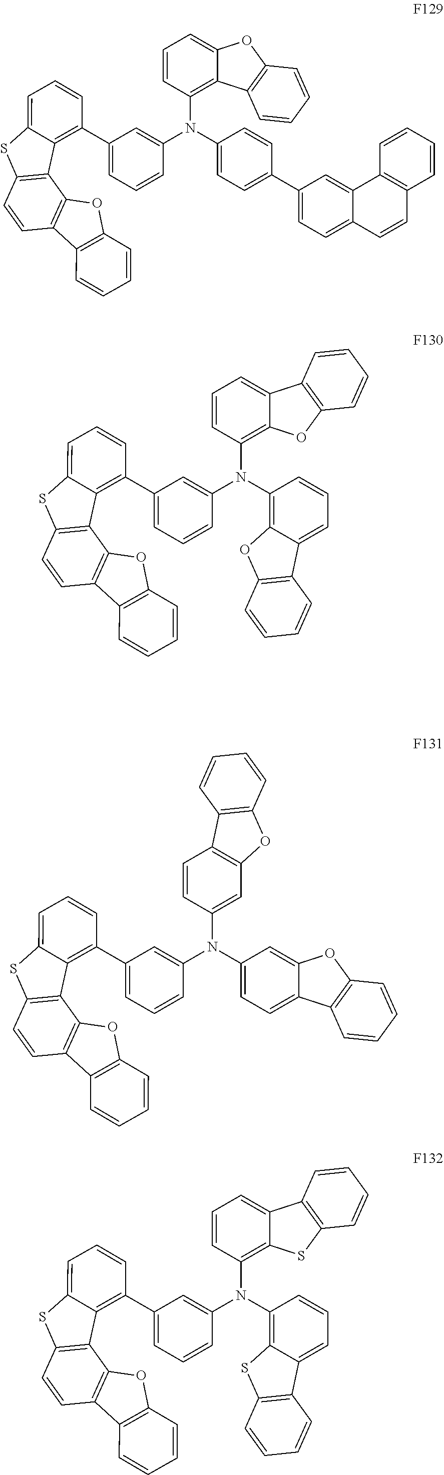 [SMILES: c1cc(-c2cccc3sc4ccc5c6ccccc6oc5c4c23)cc(N(c2ccc(-c3ccc4ccc5ccccc5c4c3)cc2)c2cccc3oc4ccccc4c23)c1 ; c1cc(-c2cccc3sc4ccc5c6ccccc6oc5c4c23)cc(N(c2cccc3c2oc2ccccc23)c2cccc3c2oc2ccccc23)c1 ; c1cc(-c2cccc3sc4ccc5c6ccccc6oc5c4c23)cc(N(c2ccc3c(c2)oc2ccccc23)c2ccc3c(c2)oc2ccccc23)c1 ; c1cc(-c2cccc3sc4ccc5c6ccccc6oc5c4c23)cc(N(c2cccc3c2sc2ccccc23)c2cccc3c2sc2ccccc23)c1]

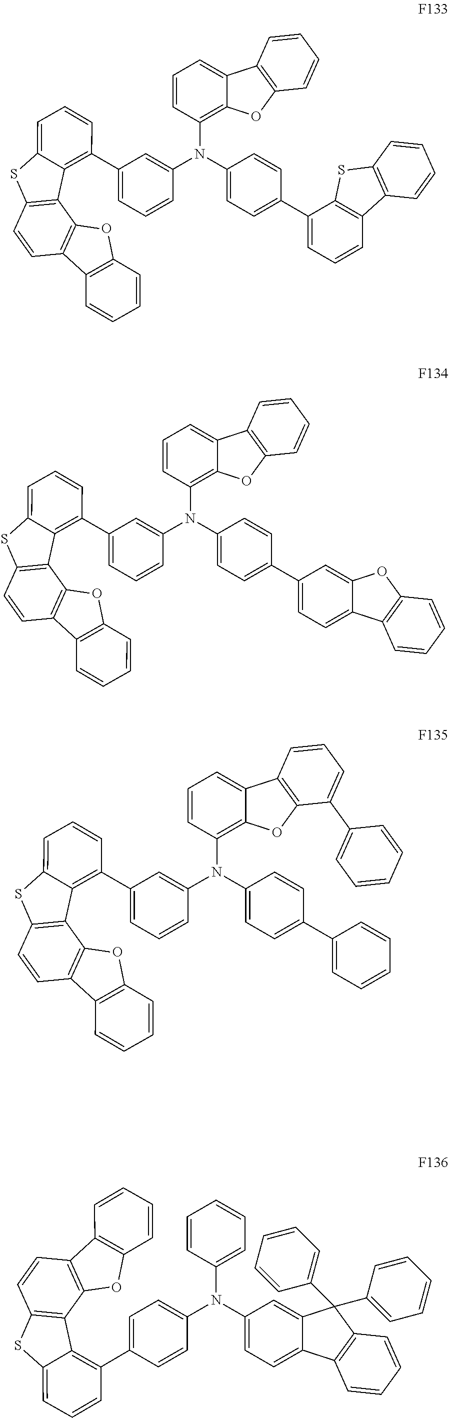 [SMILES: c1cc(-c2cccc3sc4ccc5c6ccccc6oc5c4c23)cc(N(c2ccc(-c3cccc4c3sc3ccccc34)cc2)c2cccc3c2oc2ccccc23)c1 ; c1cc(-c2cccc3sc4ccc5c6ccccc6oc5c4c23)cc(N(c2ccc(-c3ccc4c(c3)oc3ccccc34)cc2)c2cccc3c2oc2ccccc23)c1 ; c1ccc(-c2ccc(N(c3cccc(-c4cccc5sc6ccc7c8ccccc8oc7c6c45)c3)c3cccc4c3oc3c(-c5ccccc5)cccc34)cc2)cc1 ; c1ccc(N(c2ccc(-c3cccc4sc5ccc6c7ccccc7oc6c5c34)cc2)c2ccc3c(c2)C(c2ccccc2)(c2ccccc2)c2ccccc2-3)cc1]

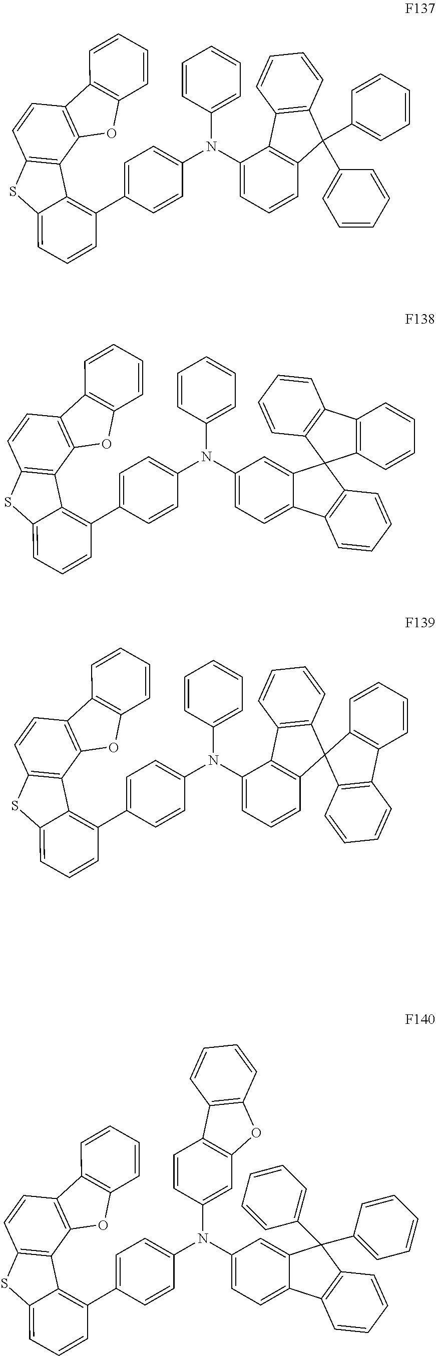 [SMILES: c1ccc(N(c2ccc(-c3cccc4sc5ccc6c7ccccc7oc6c5c34)cc2)c2cccc3c2-c2ccccc2C3(c2ccccc2)c2ccccc2)cc1 ; c1ccc(N(c2ccc(-c3cccc4sc5ccc6c7ccccc7oc6c5c34)cc2)c2ccc3c(c2)C2(c4ccccc4-c4ccccc42)c2ccccc2-3)cc1 ; c1ccc(N(c2ccc(-c3cccc4sc5ccc6c7ccccc7oc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6sc7ccc8c9ccccc9oc8c7c56)cc4)c4ccc5c(c4)oc4ccccc45)cc32)cc1]

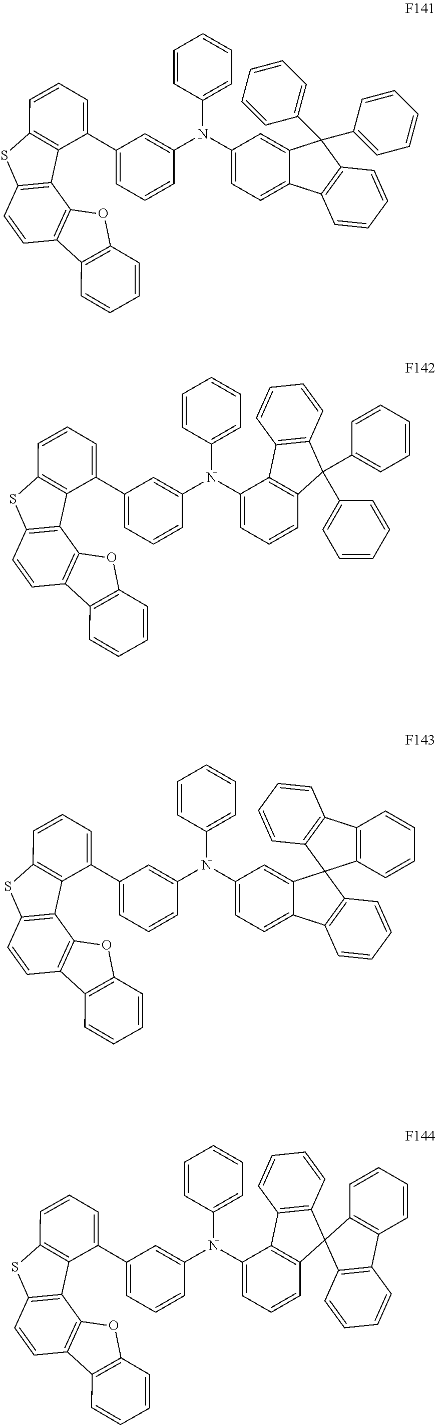 [SMILES: c1ccc(N(c2cccc(-c3cccc4sc5ccc6c7ccccc7oc6c5c34)c2)c2ccc3c(c2)C(c2ccccc2)(c2ccccc2)c2ccccc2-3)cc1 ; c1ccc(N(c2cccc(-c3cccc4sc5ccc6c7ccccc7oc6c5c34)c2)c2cccc3c2-c2ccccc2C3(c2ccccc2)c2ccccc2)cc1 ; c1ccc(N(c2cccc(-c3cccc4sc5ccc6c7ccccc7oc6c5c34)c2)c2ccc3c(c2)C2(c4ccccc4-c4ccccc42)c2ccccc2-3)cc1 ; c1ccc(N(c2cccc(-c3cccc4sc5ccc6c7ccccc7oc6c5c34)c2)c2cccc3c2-c2ccccc2C32c3ccccc3-c3ccccc32)cc1]

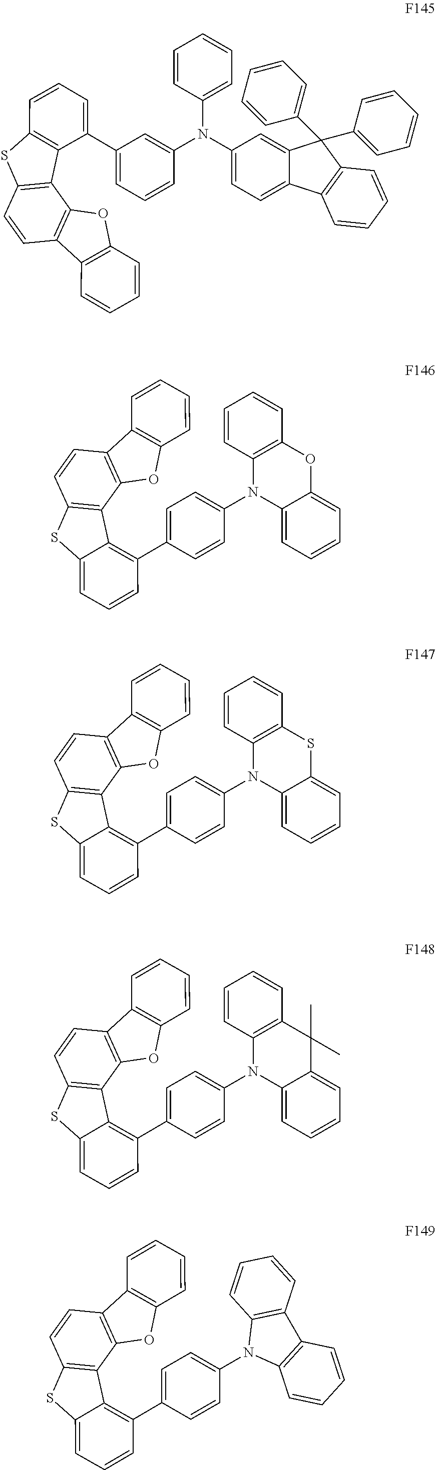 [SMILES: c1ccc(N(c2cccc(-c3cccc4sc5ccc6c7ccccc7oc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3sc4ccc5c6ccccc6oc5c4c23)cc1 ; c1ccc2c(c1)Sc1ccccc1N2c1ccc(-c2cccc3sc4ccc5c6ccccc6oc5c4c23)cc1 ; CC1(C)c2ccccc2N(c2ccc(-c3cccc4sc5ccc6c7ccccc7oc6c5c34)cc2)c2ccccc21 ; c1ccc2c(c1)oc1c2ccc2sc3cccc(-c4ccc(-n5c6ccccc6c6ccccc65)cc4)c3c21]

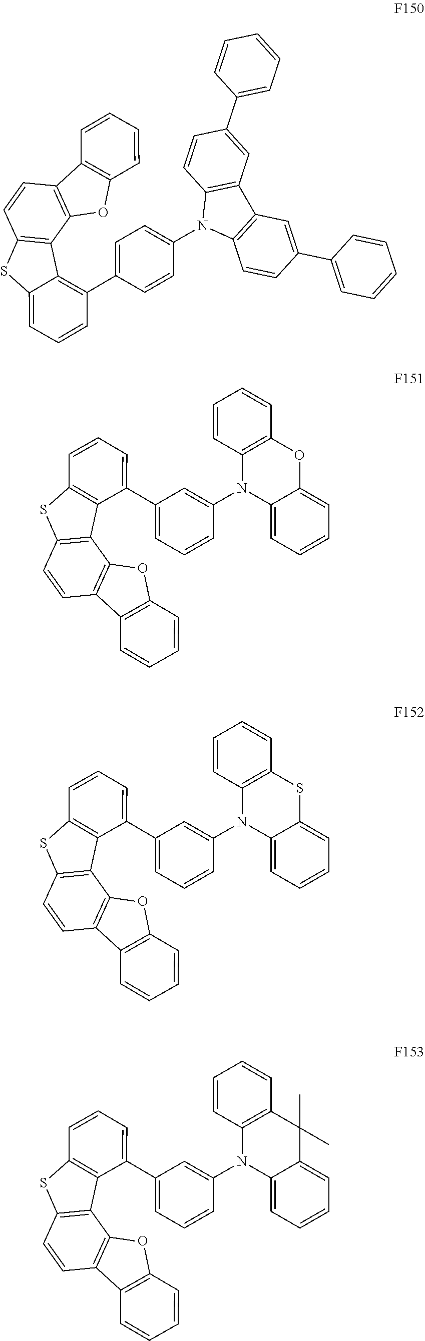 [SMILES: c1ccc(-c2ccc3c(c2)c2cc(-c4ccccc4)ccc2n3-c2ccc(-c3cccc4sc5ccc6c7ccccc7oc6c5c34)cc2)cc1 ; c1cc(-c2cccc3sc4ccc5c6ccccc6oc5c4c23)cc(N2c3ccccc3Oc3ccccc32)c1 ; c1cc(-c2cccc3sc4ccc5c6ccccc6oc5c4c23)cc(N2c3ccccc3Sc3ccccc32)c1 ; CC1(C)c2ccccc2N(c2cccc(-c3cccc4sc5ccc6c7ccccc7oc6c5c34)c2)c2ccccc21]

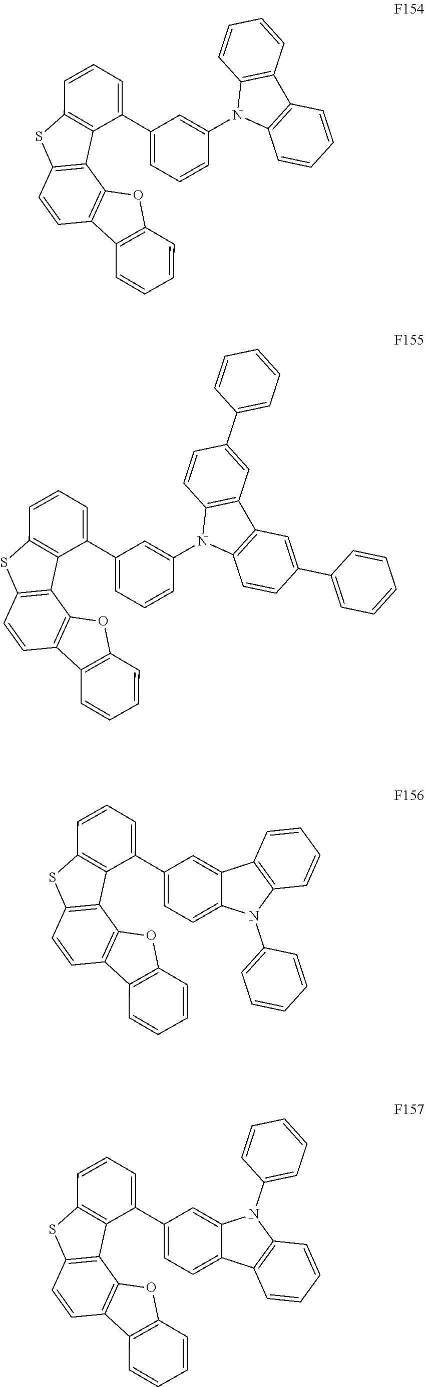 [SMILES: c1cc(-c2cccc3sc4ccc5c6ccccc6oc5c4c23)cc(-n2c3ccccc3c3ccccc32)c1 ; c1ccc(-c2ccc3c(c2)c2cc(-c4ccccc4)ccc2n3-c2cccc(-c3cccc4sc5ccc6c7ccccc7oc6c5c34)c2)cc1 ; c1ccc(-n2c3ccccc3c3cc(-c4cccc5sc6ccc7c8ccccc8oc7c6c45)ccc32)cc1 ; c1ccc(-n2c3ccccc3c3ccc(-c4cccc5sc6ccc7c8ccccc8oc7c6c45)cc32)cc1]

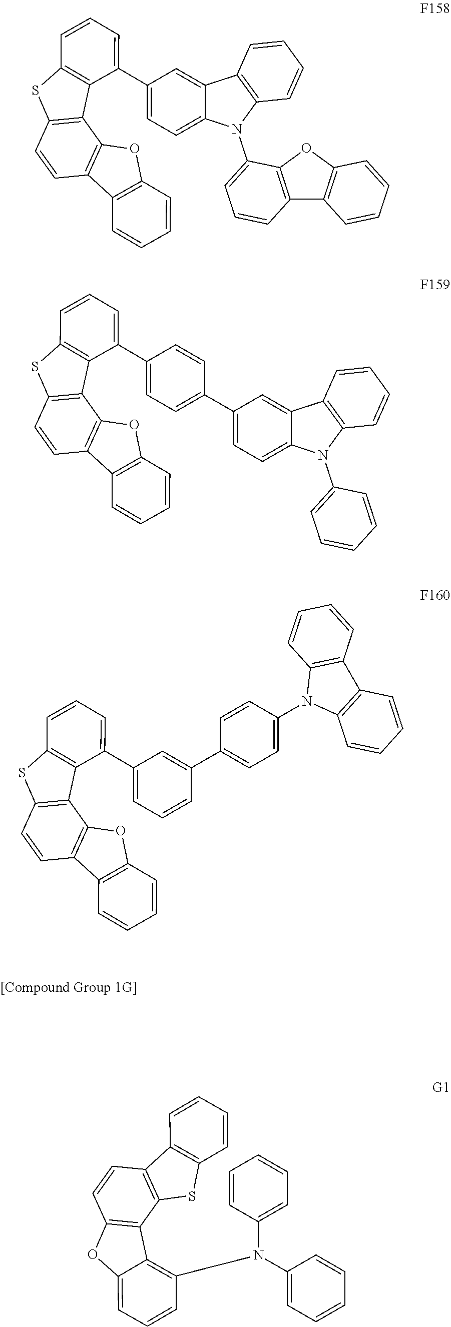 [SMILES: c1ccc2c(c1)oc1c(-n3c4ccccc4c4cc(-c5cccc6sc7ccc8c9ccccc9oc8c7c56)ccc43)cccc12 ; c1ccc(-n2c3ccccc3c3cc(-c4ccc(-c5cccc6sc7ccc8c9ccccc9oc8c7c56)cc4)ccc32)cc1 ; c1cc(-c2ccc(-n3c4ccccc4c4ccccc43)cc2)cc(-c2cccc3sc4ccc5c6ccccc6oc5c4c23)c1 ; c1ccc(N(c2ccccc2)c2cccc3oc4ccc5c6ccccc6sc5c4c23)cc1]

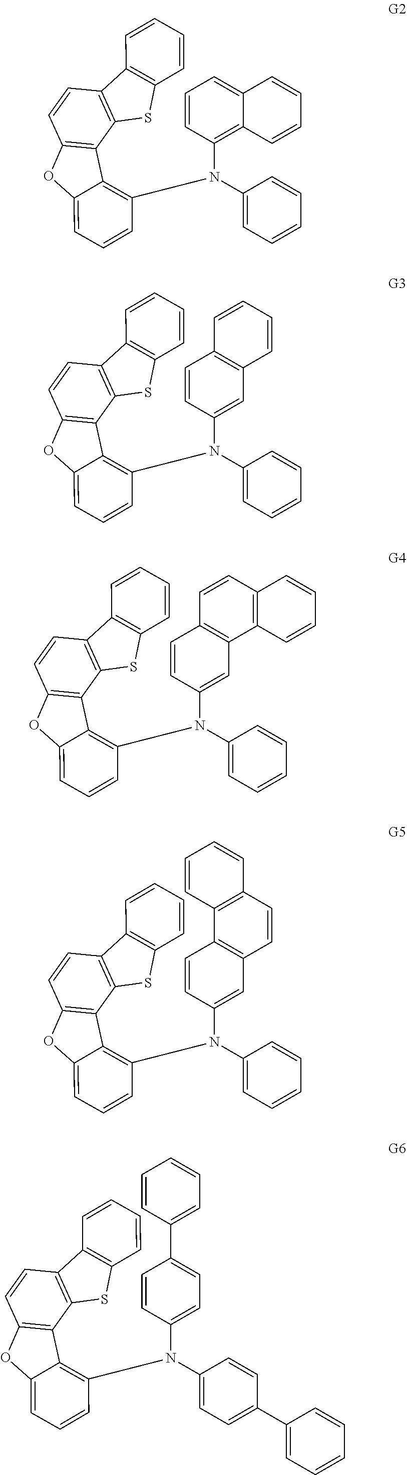 [SMILES: c1ccc(N(c2cccc3ccccc23)c2cccc3oc4ccc5c6ccccc6sc5c4c23)cc1 ; c1ccc(N(c2ccc3ccccc3c2)c2cccc3oc4ccc5c6ccccc6sc5c4c23)cc1 ; c1ccc(N(c2ccc3ccc4ccccc4c3c2)c2cccc3oc4ccc5c6ccccc6sc5c4c23)cc1 ; c1ccc(N(c2ccc3c(ccc4ccccc43)c2)c2cccc3oc4ccc5c6ccccc6sc5c4c23)cc1 ; c1ccc(-c2ccc(N(c3ccc(-c4ccccc4)cc3)c3cccc4oc5ccc6c7ccccc7sc6c5c34)cc2)cc1]

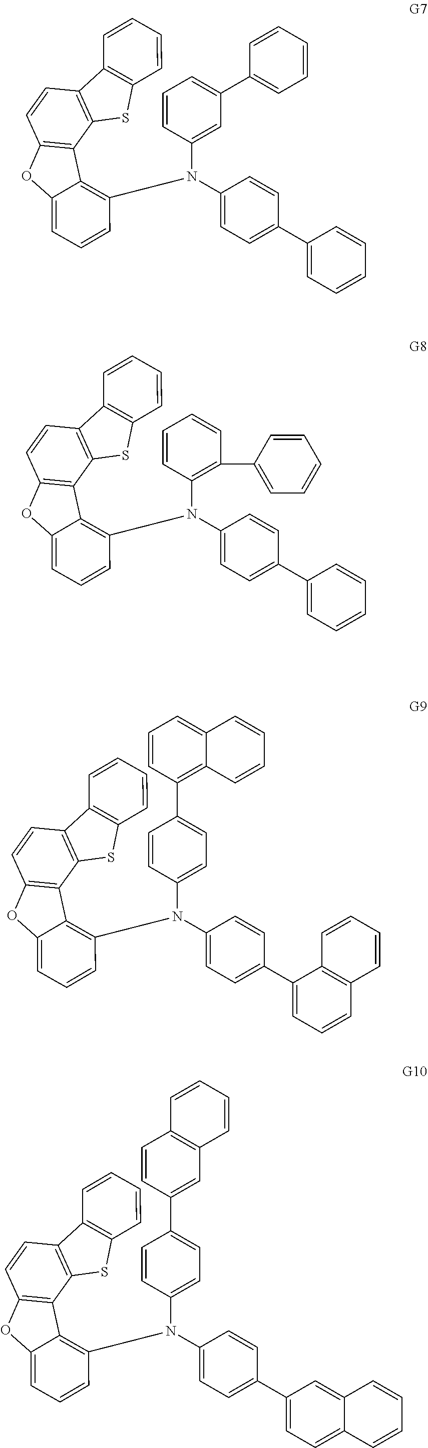 [SMILES: c1ccc(-c2ccc(N(c3cccc(-c4ccccc4)c3)c3cccc4oc5ccc6c7ccccc7sc6c5c34)cc2)cc1 ; c1ccc(-c2ccc(N(c3ccccc3-c3ccccc3)c3cccc4oc5ccc6c7ccccc7sc6c5c34)cc2)cc1 ; c1ccc2c(-c3ccc(N(c4ccc(-c5cccc6ccccc56)cc4)c4cccc5oc6ccc7c8ccccc8sc7c6c45)cc3)cccc2c1 ; c1ccc2cc(-c3ccc(N(c4ccc(-c5ccc6ccccc6c5)cc4)c4cccc5oc6ccc7c8ccccc8sc7c6c45)cc3)ccc2c1]

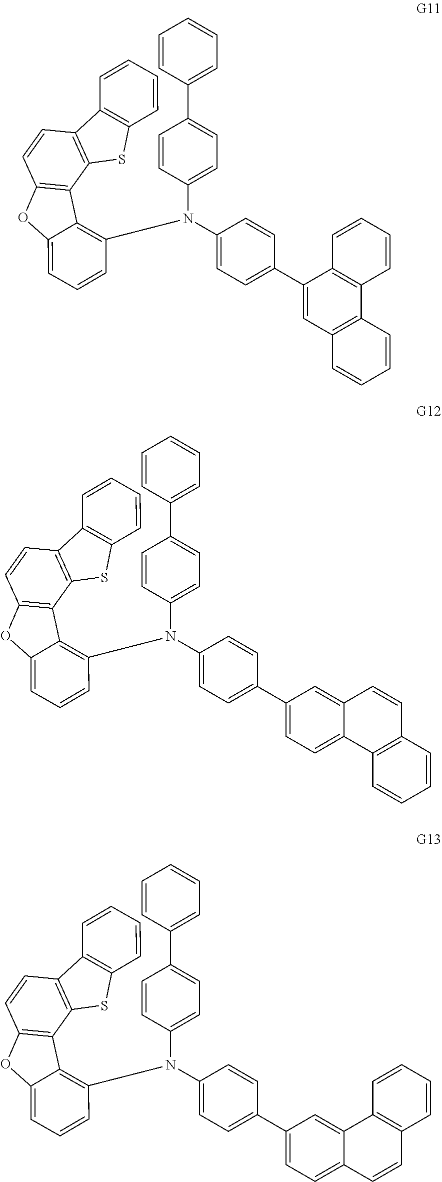 [SMILES: c1ccc(-c2ccc(N(c3ccc(-c4cc5ccccc5c5ccccc45)cc3)c3cccc4oc5ccc6c7ccccc7sc6c5c34)cc2)cc1 ; c1ccc(-c2ccc(N(c3ccc(-c4ccc5c(ccc6ccccc65)c4)cc3)c3cccc4oc5ccc6c7ccccc7sc6c5c34)cc2)cc1 ; c1ccc(-c2ccc(N(c3ccc(-c4ccc5ccc6ccccc6c5c4)cc3)c3cccc4oc5ccc6c7ccccc7sc6c5c34)cc2)cc1]

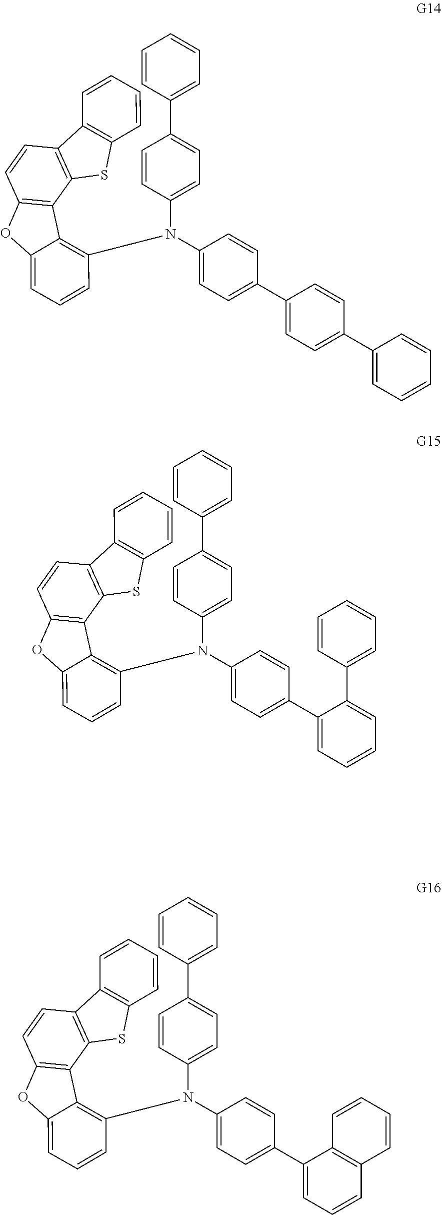 [SMILES: c1ccc(-c2ccc(-c3ccc(N(c4ccc(-c5ccccc5)cc4)c4cccc5oc6ccc7c8ccccc8sc7c6c45)cc3)cc2)cc1 ; c1ccc(-c2ccc(N(c3ccc(-c4ccccc4-c4ccccc4)cc3)c3cccc4oc5ccc6c7ccccc7sc6c5c34)cc2)cc1 ; c1ccc(-c2ccc(N(c3ccc(-c4cccc5ccccc45)cc3)c3cccc4oc5ccc6c7ccccc7sc6c5c34)cc2)cc1]

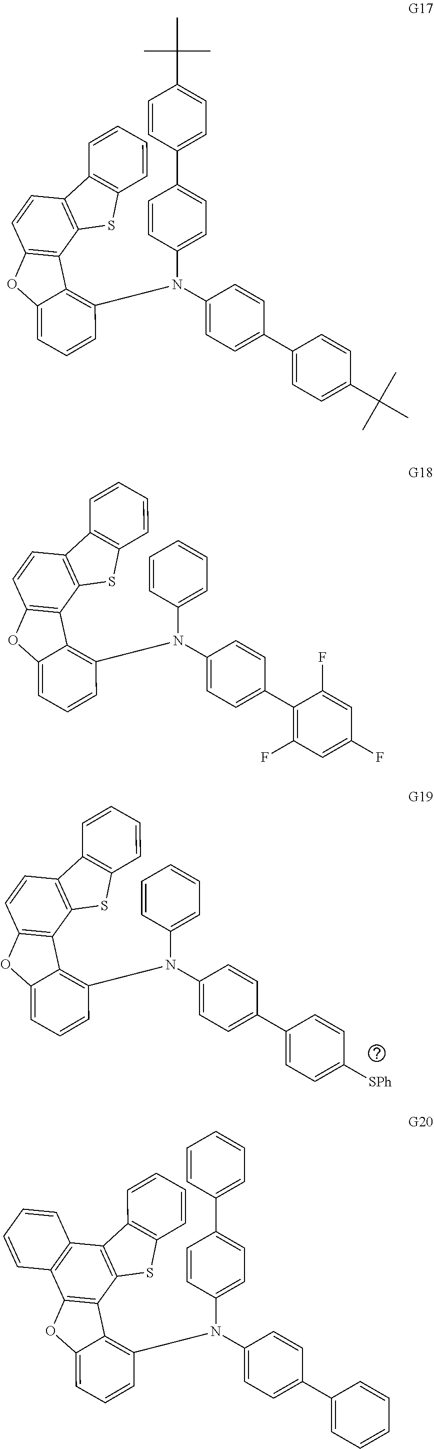

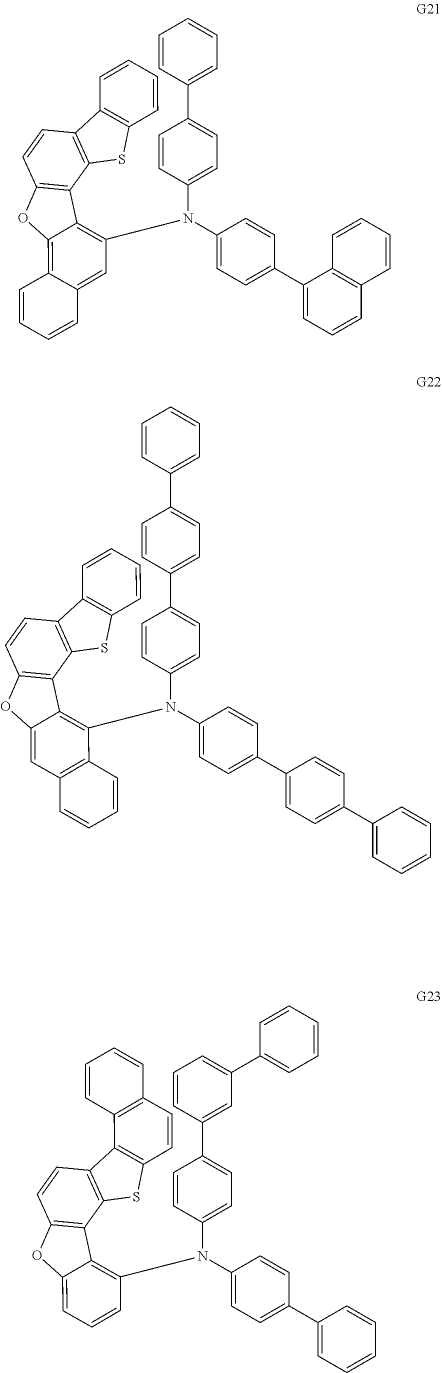 [SMILES: c1ccc(-c2ccc(N(c3ccc(-c4cccc5ccccc45)cc3)c3cc4ccccc4c4oc5ccc6c7ccccc7sc6c5c34)cc2)cc1 ; c1ccc(-c2ccc(-c3ccc(N(c4ccc(-c5ccc(-c6ccccc6)cc5)cc4)c4c5ccccc5cc5oc6ccc7c8ccccc8sc7c6c45)cc3)cc2)cc1 ; c1ccc(-c2ccc(N(c3ccc(-c4cccc(-c5ccccc5)c4)cc3)c3cccc4oc5ccc6c(sc7ccc8ccccc8c76)c5c34)cc2)cc1]

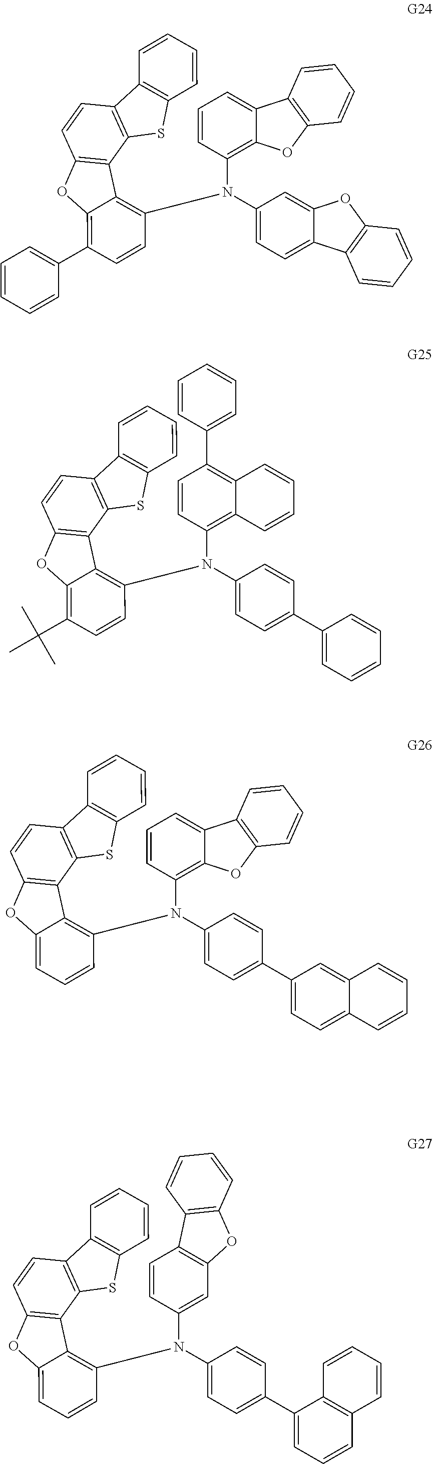 [SMILES: c1ccc(-c2ccc(N(c3ccc4c(c3)oc3ccccc34)c3cccc4c3oc3ccccc34)c3c2oc2ccc4c5ccccc5sc4c23)cc1 ; CC(C)(C)c1ccc(N(c2ccc(-c3ccccc3)cc2)c2ccc(-c3ccccc3)c3ccccc23)c2c1oc1ccc3c4ccccc4sc3c12 ; c1ccc2cc(-c3ccc(N(c4cccc5c4oc4ccccc45)c4cccc5oc6ccc7c8ccccc8sc7c6c45)cc3)ccc2c1 ; c1ccc2c(-c3ccc(N(c4ccc5c(c4)oc4ccccc45)c4cccc5oc6ccc7c8ccccc8sc7c6c45)cc3)cccc2c1]

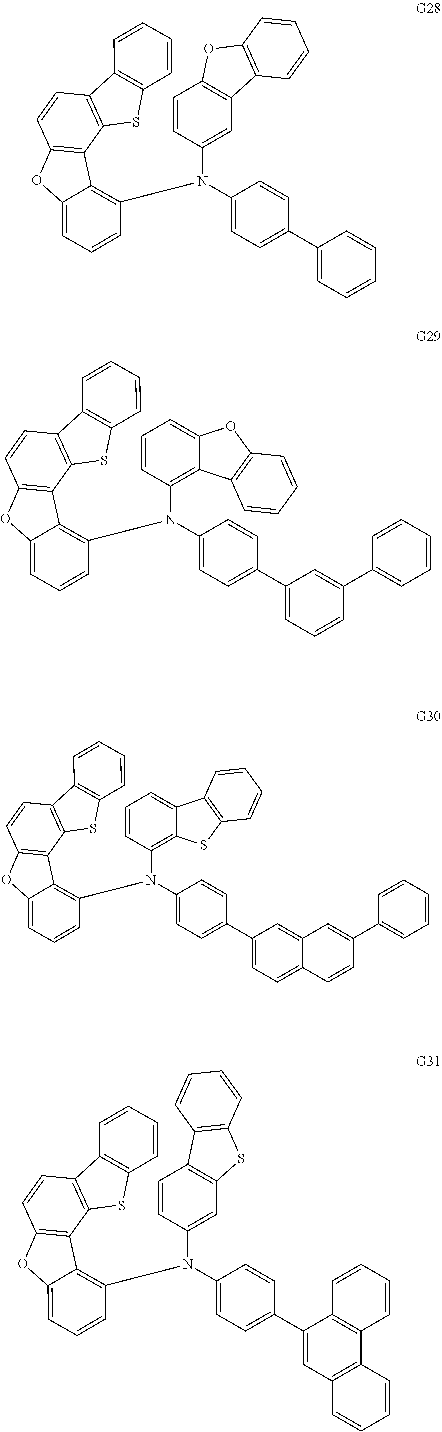 [SMILES: c1ccc(-c2ccc(N(c3ccc4oc5ccccc5c4c3)c3cccc4oc5ccc6c7ccccc7sc6c5c34)cc2)cc1 ; c1ccc(-c2cccc(-c3ccc(N(c4cccc5oc6ccccc6c45)c4cccc5oc6ccc7c8ccccc8sc7c6c45)cc3)c2)cc1 ; c1ccc(-c2ccc3ccc(-c4ccc(N(c5cccc6c5sc5ccccc56)c5cccc6oc7ccc8c9ccccc9sc8c7c56)cc4)cc3c2)cc1 ; c1ccc2c(c1)cc(-c1ccc(N(c3ccc4c(c3)sc3ccccc34)c3cccc4oc5ccc6c7ccccc7sc6c5c34)cc1)c1ccccc12]

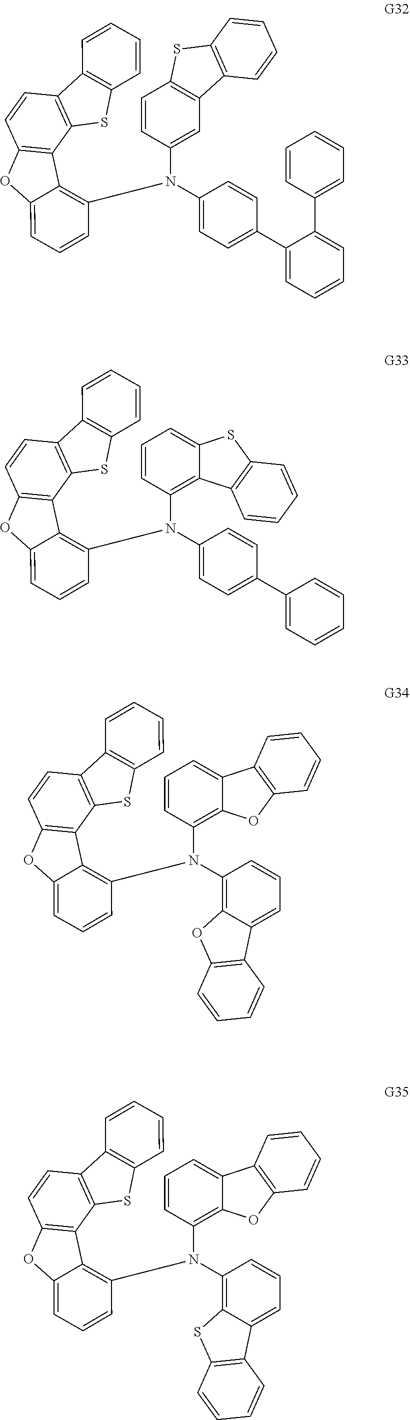 [SMILES: c1ccc(-c2ccccc2-c2ccc(N(c3ccc4sc5ccccc5c4c3)c3cccc4oc5ccc6c7ccccc7sc6c5c34)cc2)cc1 ; c1ccc(-c2ccc(N(c3cccc4sc5ccccc5c34)c3cccc4oc5ccc6c7ccccc7sc6c5c34)cc2)cc1 ; c1ccc2c(c1)oc1c(N(c3cccc4c3oc3ccccc34)c3cccc4oc5ccc6c7ccccc7sc6c5c34)cccc12 ; c1ccc2c(c1)oc1c(N(c3cccc4c3sc3ccccc34)c3cccc4oc5ccc6c7ccccc7sc6c5c34)cccc12]

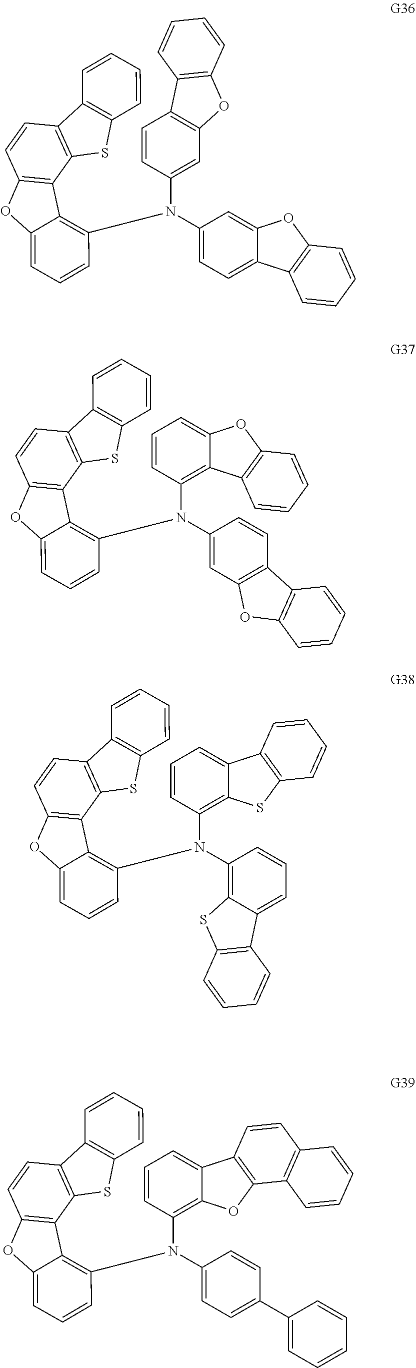 [SMILES: c1ccc2c(c1)oc1cc(N(c3ccc4c(c3)oc3ccccc34)c3cccc4oc5ccc6c7ccccc7sc6c5c34)ccc12 ; c1ccc2c(c1)oc1cc(N(c3cccc4oc5ccccc5c34)c3cccc4oc5ccc6c7ccccc7sc6c5c34)ccc12 ; c1cc(N(c2cccc3c2sc2ccccc23)c2cccc3c2sc2ccccc23)c2c(c1)oc1ccc3c4ccccc4sc3c12 ; c1ccc(-c2ccc(N(c3cccc4c3oc3c5ccccc5ccc43)c3cccc4oc5ccc6c7ccccc7sc6c5c34)cc2)cc1]

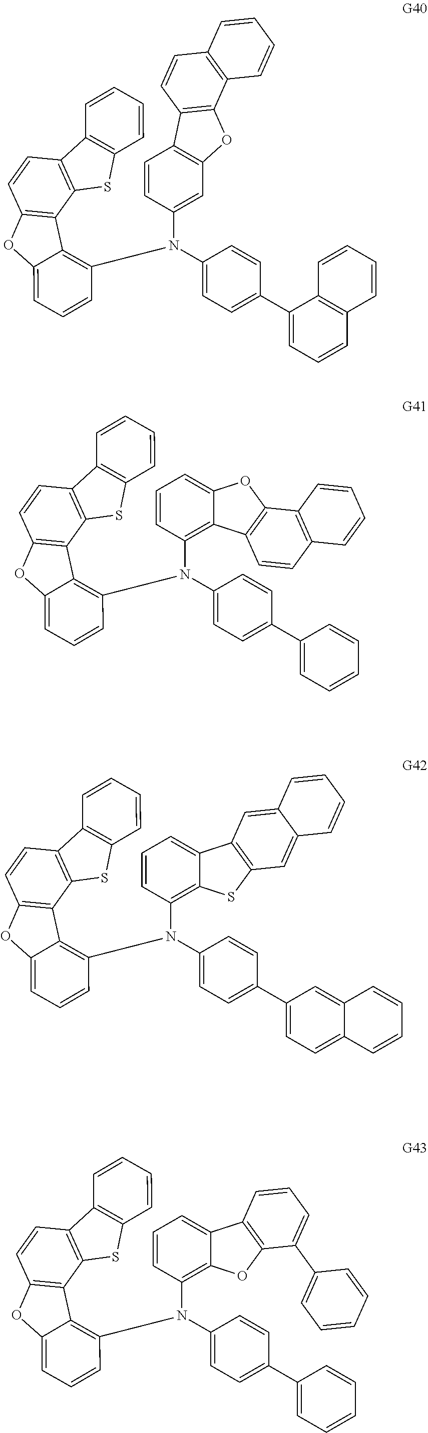 [SMILES: c1ccc2c(-c3ccc(N(c4ccc5c(c4)oc4c6ccccc6ccc54)c4cccc5oc6ccc7c8ccccc8sc7c6c45)cc3)cccc2c1 ; c1ccc(-c2ccc(N(c3cccc4oc5c6ccccc6ccc5c34)c3cccc4oc5ccc6c7ccccc7sc6c5c34)cc2)cc1 ; c1ccc2cc(-c3ccc(N(c4cccc5c4sc4cc6ccccc6cc45)c4cccc5oc6ccc7c8ccccc8sc7c6c45)cc3)ccc2c1 ; c1ccc(-c2ccc(N(c3cccc4c3oc3c(-c5ccccc5)cccc34)c3cccc4oc5ccc6c7ccccc7sc6c5c34)cc2)cc1]

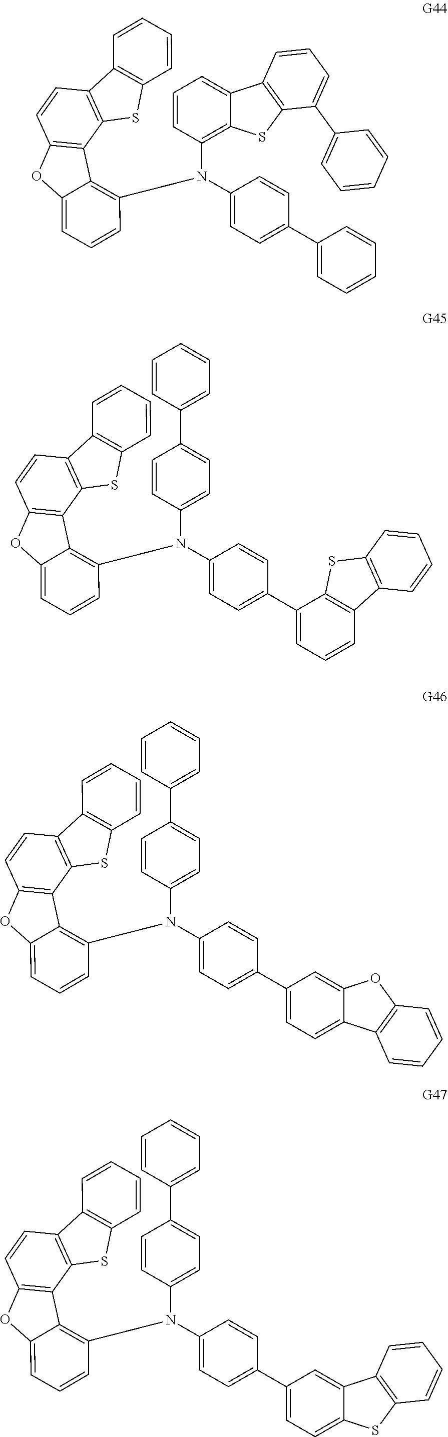 [SMILES: c1ccc(-c2ccc(N(c3cccc4c3sc3c(-c5ccccc5)cccc34)c3cccc4oc5ccc6c7ccccc7sc6c5c34)cc2)cc1 ; c1ccc(-c2ccc(N(c3ccc(-c4cccc5c4sc4ccccc45)cc3)c3cccc4oc5ccc6c7ccccc7sc6c5c34)cc2)cc1 ; c1ccc(-c2ccc(N(c3ccc(-c4ccc5c(c4)oc4ccccc45)cc3)c3cccc4oc5ccc6c7ccccc7sc6c5c34)cc2)cc1 ; c1ccc(-c2ccc(N(c3ccc(-c4ccc5sc6ccccc6c5c4)cc3)c3cccc4oc5ccc6c7ccccc7sc6c5c34)cc2)cc1]

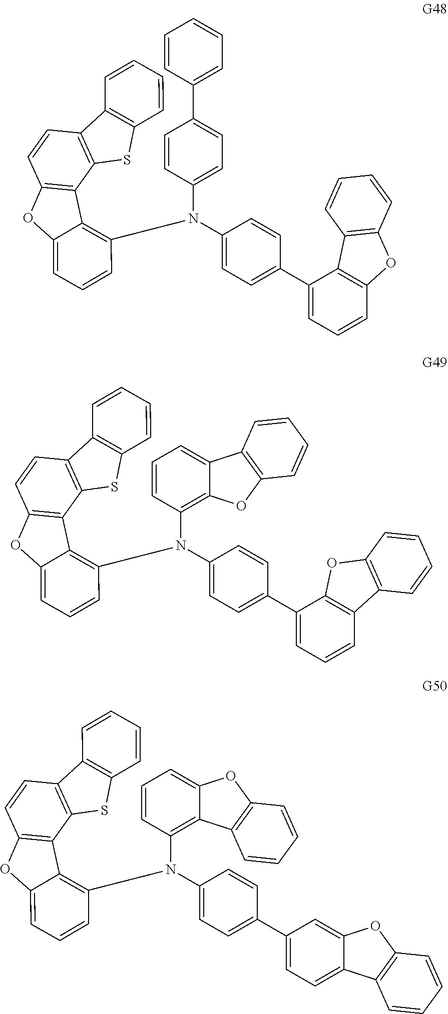 [SMILES: c1ccc(-c2ccc(N(c3ccc(-c4cccc5oc6ccccc6c45)cc3)c3cccc4oc5ccc6c7ccccc7sc6c5c34)cc2)cc1 ; c1ccc2c(c1)oc1c(-c3ccc(N(c4cccc5c4oc4ccccc45)c4cccc5oc6ccc7c8ccccc8sc7c6c45)cc3)cccc12 ; c1ccc2c(c1)oc1cc(-c3ccc(N(c4cccc5oc6ccccc6c45)c4cccc5oc6ccc7c8ccccc8sc7c6c45)cc3)ccc12]

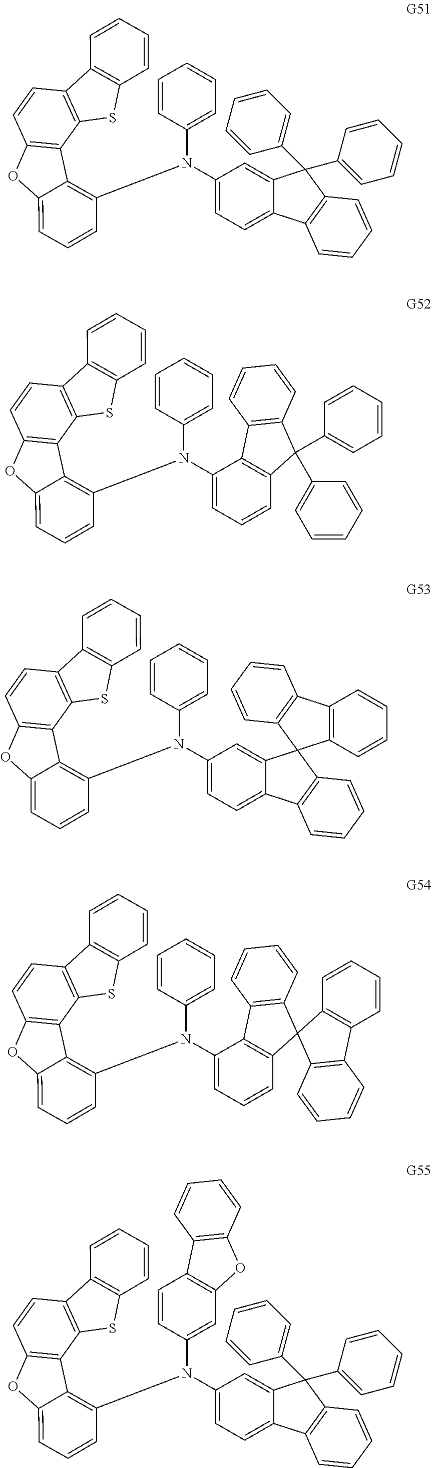 [SMILES: c1ccc(N(c2ccc3c(c2)C(c2ccccc2)(c2ccccc2)c2ccccc2-3)c2cccc3oc4ccc5c6ccccc6sc5c4c23)cc1 ; c1ccc(N(c2cccc3c2-c2ccccc2C3(c2ccccc2)c2ccccc2)c2cccc3oc4ccc5c6ccccc6sc5c4c23)cc1 ; c1ccc(N(c2ccc3c(c2)C2(c4ccccc4-c4ccccc42)c2ccccc2-3)c2cccc3oc4ccc5c6ccccc6sc5c4c23)cc1 ; c1ccc(N(c2cccc3c2-c2ccccc2C32c3ccccc3-c3ccccc32)c2cccc3oc4ccc5c6ccccc6sc5c4c23)cc1 ; c1ccc(C2(c3ccccc3)c3ccccc3-c3ccc(N(c4ccc5c(c4)oc4ccccc45)c4cccc5oc6ccc7c8ccccc8sc7c6c45)cc32)cc1]

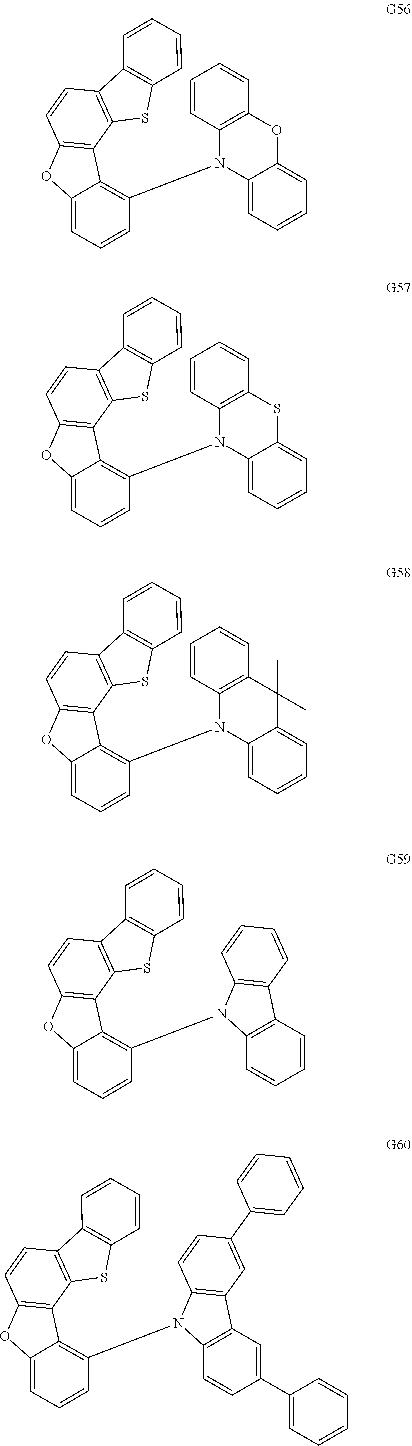 [SMILES: c1ccc2c(c1)Oc1ccccc1N2c1cccc2oc3ccc4c5ccccc5sc4c3c12 ; c1ccc2c(c1)Sc1ccccc1N2c1cccc2oc3ccc4c5ccccc5sc4c3c12 ; CC1(C)c2ccccc2N(c2cccc3oc4ccc5c6ccccc6sc5c4c23)c2ccccc21 ; c1cc(-n2c3ccccc3c3ccccc32)c2c(c1)oc1ccc3c4ccccc4sc3c12 ; c1ccc(-c2ccc3c(c2)c2cc(-c4ccccc4)ccc2n3-c2cccc3oc4ccc5c6ccccc6sc5c4c23)cc1]

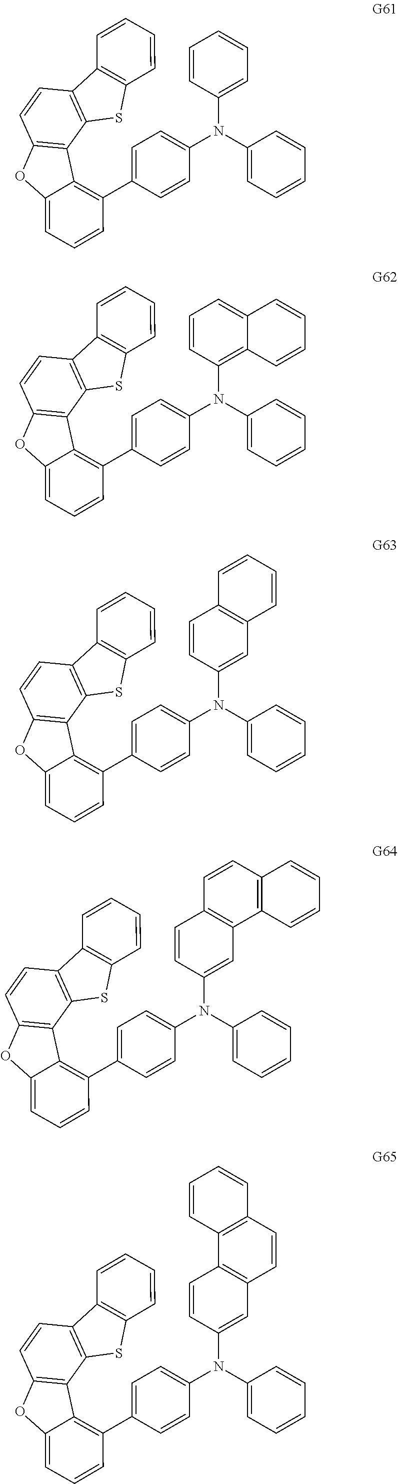 [SMILES: c1ccc(N(c2ccccc2)c2ccc(-c3cccc4oc5ccc6c7ccccc7sc6c5c34)cc2)cc1 ; c1ccc(N(c2ccc(-c3cccc4oc5ccc6c7ccccc7sc6c5c34)cc2)c2cccc3ccccc23)cc1 ; c1ccc(N(c2ccc(-c3cccc4oc5ccc6c7ccccc7sc6c5c34)cc2)c2ccc3ccccc3c2)cc1 ; c1ccc(N(c2ccc(-c3cccc4oc5ccc6c7ccccc7sc6c5c34)cc2)c2ccc3ccc4ccccc4c3c2)cc1 ; c1ccc(N(c2ccc(-c3cccc4oc5ccc6c7ccccc7sc6c5c34)cc2)c2ccc3c(ccc4ccccc43)c2)cc1]

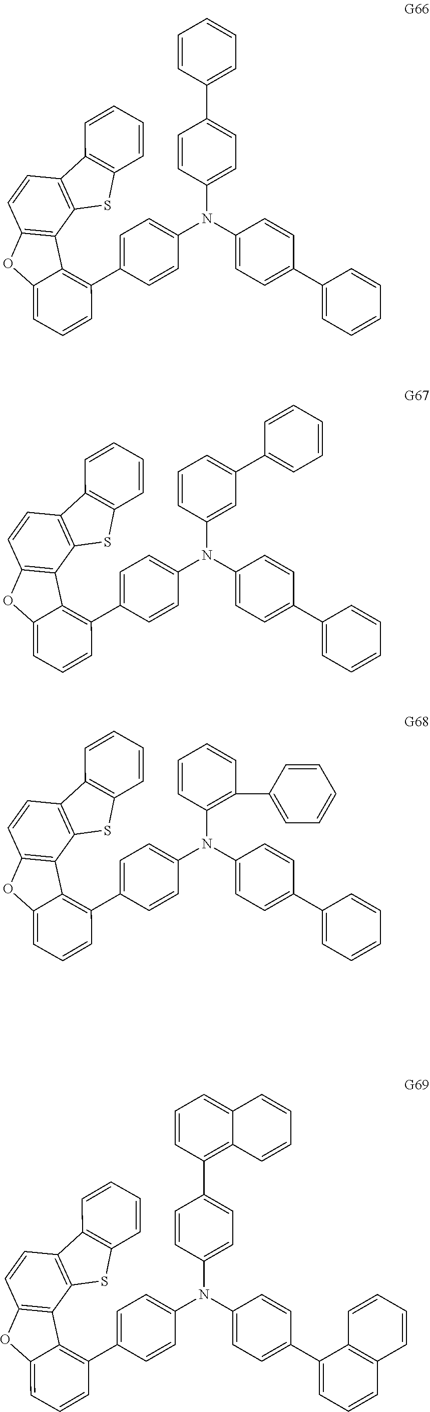 [SMILES: c1ccc(-c2ccc(N(c3ccc(-c4ccccc4)cc3)c3ccc(-c4cccc5oc6ccc7c8ccccc8sc7c6c45)cc3)cc2)cc1 ; c1ccc(-c2ccc(N(c3ccc(-c4cccc5oc6ccc7c8ccccc8sc7c6c45)cc3)c3cccc(-c4ccccc4)c3)cc2)cc1 ; c1ccc(-c2ccc(N(c3ccc(-c4cccc5oc6ccc7c8ccccc8sc7c6c45)cc3)c3ccccc3-c3ccccc3)cc2)cc1 ; c1ccc2c(-c3ccc(N(c4ccc(-c5cccc6ccccc56)cc4)c4ccc(-c5cccc6oc7ccc8c9ccccc9sc8c7c56)cc4)cc3)cccc2c1]

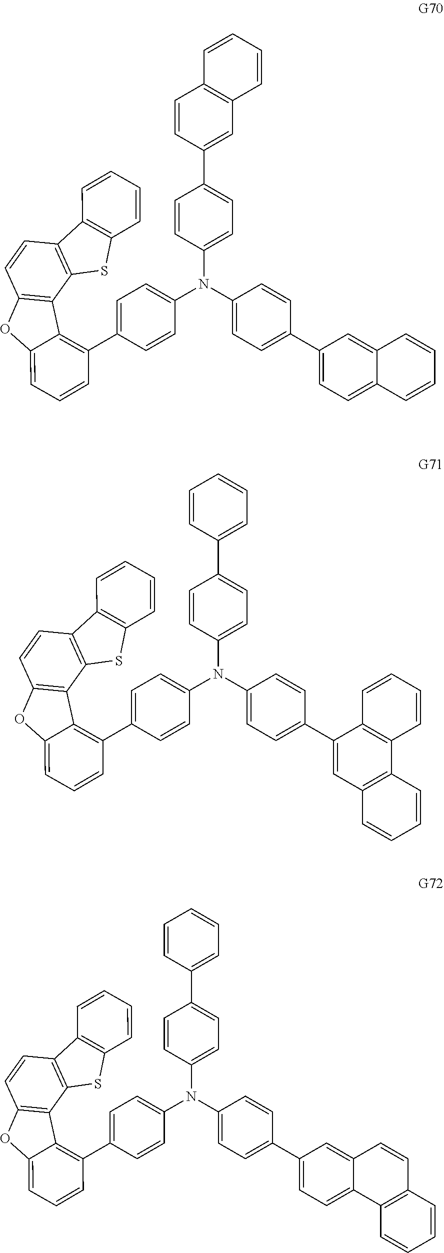 [SMILES: c1ccc2cc(-c3ccc(N(c4ccc(-c5ccc6ccccc6c5)cc4)c4ccc(-c5cccc6oc7ccc8c9ccccc9sc8c7c56)cc4)cc3)ccc2c1 ; c1ccc(-c2ccc(N(c3ccc(-c4cc5ccccc5c5ccccc45)cc3)c3ccc(-c4cccc5oc6ccc7c8ccccc8sc7c6c45)cc3)cc2)cc1 ; c1ccc(-c2ccc(N(c3ccc(-c4ccc5c(ccc6ccccc65)c4)cc3)c3ccc(-c4cccc5oc6ccc7c8ccccc8sc7c6c45)cc3)cc2)cc1]

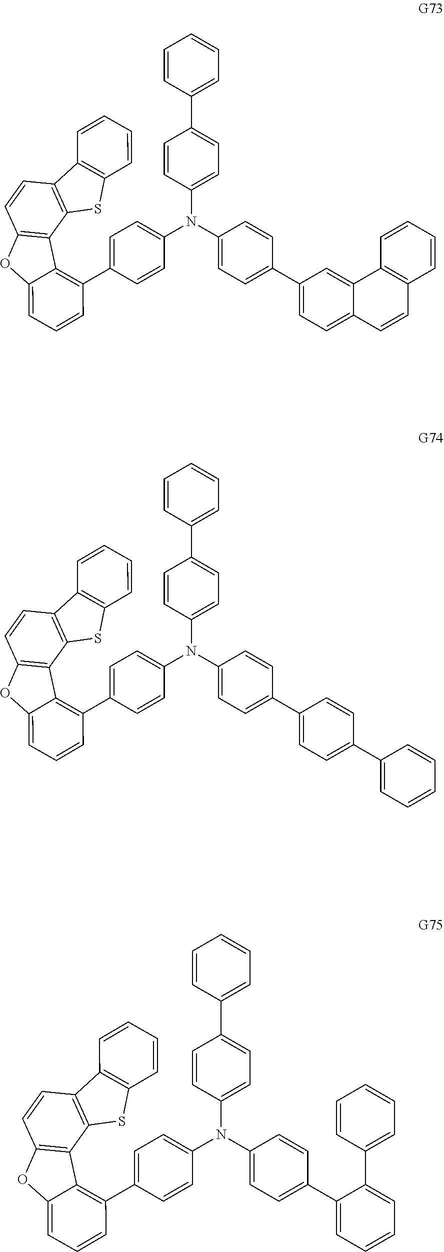 [SMILES: c1ccc(-c2ccc(N(c3ccc(-c4ccc5ccc6ccccc6c5c4)cc3)c3ccc(-c4cccc5oc6ccc7c8ccccc8sc7c6c45)cc3)cc2)cc1 ; c1ccc(-c2ccc(-c3ccc(N(c4ccc(-c5ccccc5)cc4)c4ccc(-c5cccc6oc7ccc8c9ccccc9sc8c7c56)cc4)cc3)cc2)cc1 ; c1ccc(-c2ccc(N(c3ccc(-c4ccccc4-c4ccccc4)cc3)c3ccc(-c4cccc5oc6ccc7c8ccccc8sc7c6c45)cc3)cc2)cc1]

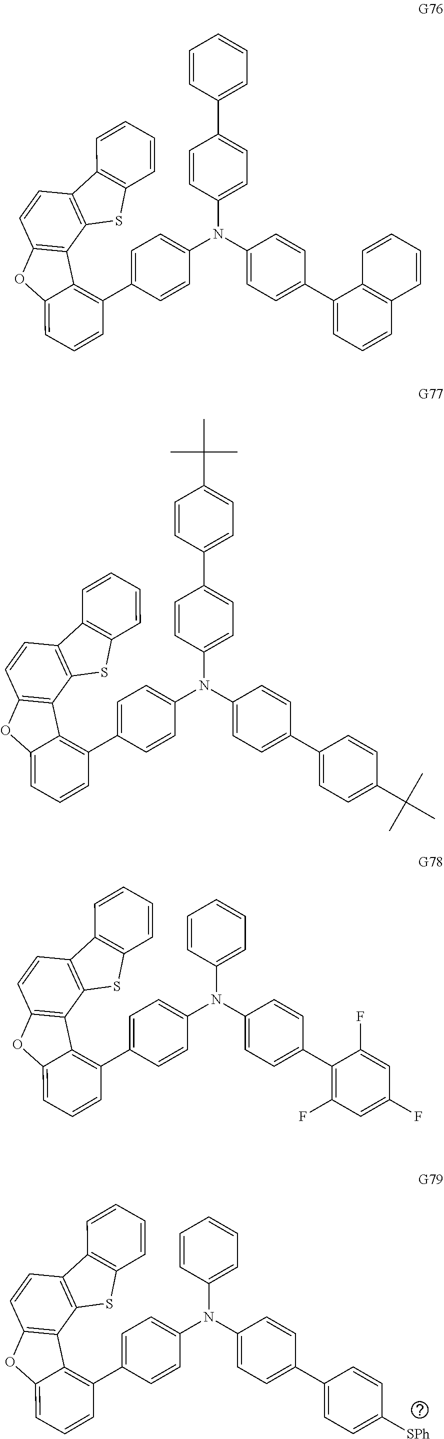

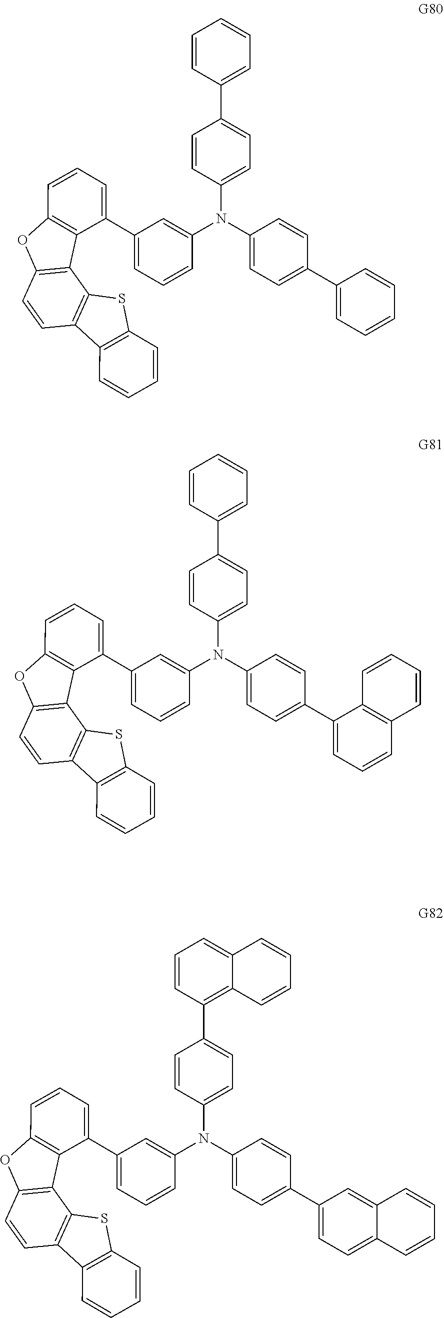 [SMILES: c1ccc(-c2ccc(N(c3ccc(-c4ccccc4)cc3)c3cccc(-c4cccc5oc6ccc7c8ccccc8sc7c6c45)c3)cc2)cc1 ; c1ccc(-c2ccc(N(c3ccc(-c4cccc5ccccc45)cc3)c3cccc(-c4cccc5oc6ccc7c8ccccc8sc7c6c45)c3)cc2)cc1 ; c1cc(-c2cccc3oc4ccc5c6ccccc6sc5c4c23)cc(N(c2ccc(-c3ccc4ccccc4c3)cc2)c2ccc(-c3cccc4ccccc34)cc2)c1]

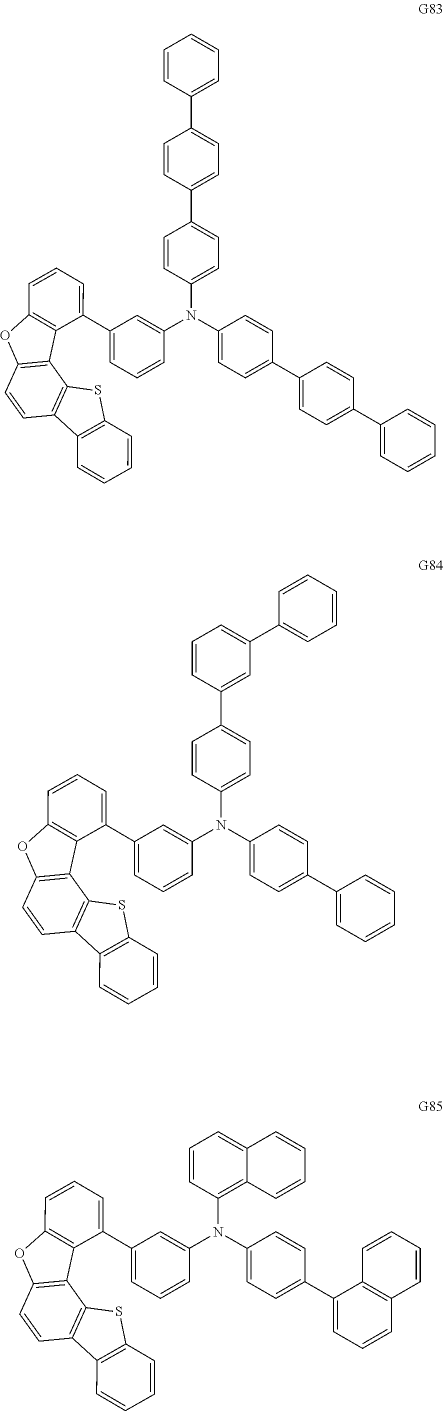 [SMILES: c1ccc(-c2ccc(-c3ccc(N(c4ccc(-c5ccc(-c6ccccc6)cc5)cc4)c4cccc(-c5cccc6oc7ccc8c9ccccc9sc8c7c56)c4)cc3)cc2)cc1 ; c1ccc(-c2ccc(N(c3ccc(-c4cccc(-c5ccccc5)c4)cc3)c3cccc(-c4cccc5oc6ccc7c8ccccc8sc7c6c45)c3)cc2)cc1 ; c1cc(-c2cccc3oc4ccc5c6ccccc6sc5c4c23)cc(N(c2ccc(-c3cccc4ccccc34)cc2)c2cccc3ccccc23)c1]

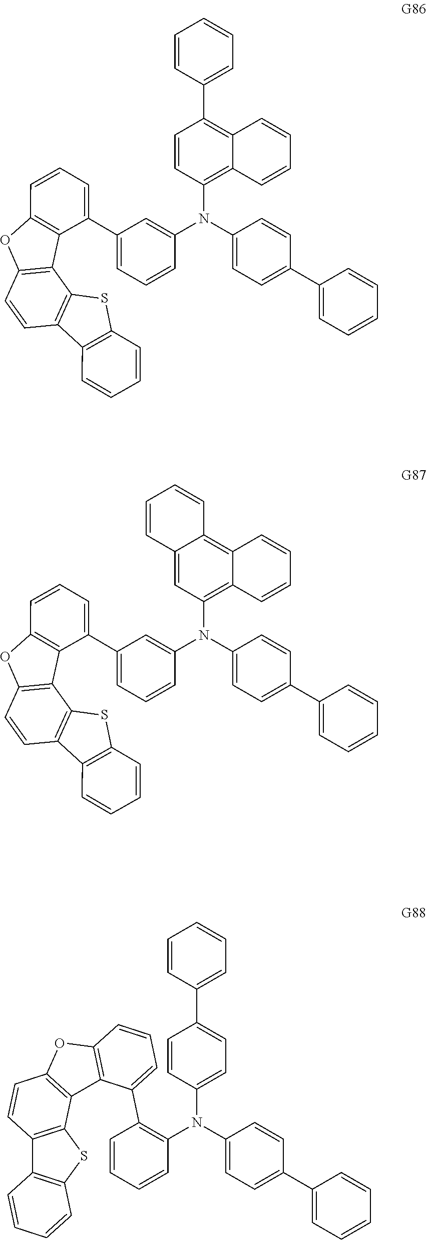 [SMILES: c1ccc(-c2ccc(N(c3cccc(-c4cccc5oc6ccc7c8ccccc8sc7c6c45)c3)c3ccc(-c4ccccc4)c4ccccc34)cc2)cc1 ; c1ccc(-c2ccc(N(c3cccc(-c4cccc5oc6ccc7c8ccccc8sc7c6c45)c3)c3cc4ccccc4c4ccccc34)cc2)cc1 ; c1ccc(-c2ccc(N(c3ccc(-c4ccccc4)cc3)c3ccccc3-c3cccc4oc5ccc6c7ccccc7sc6c5c34)cc2)cc1]

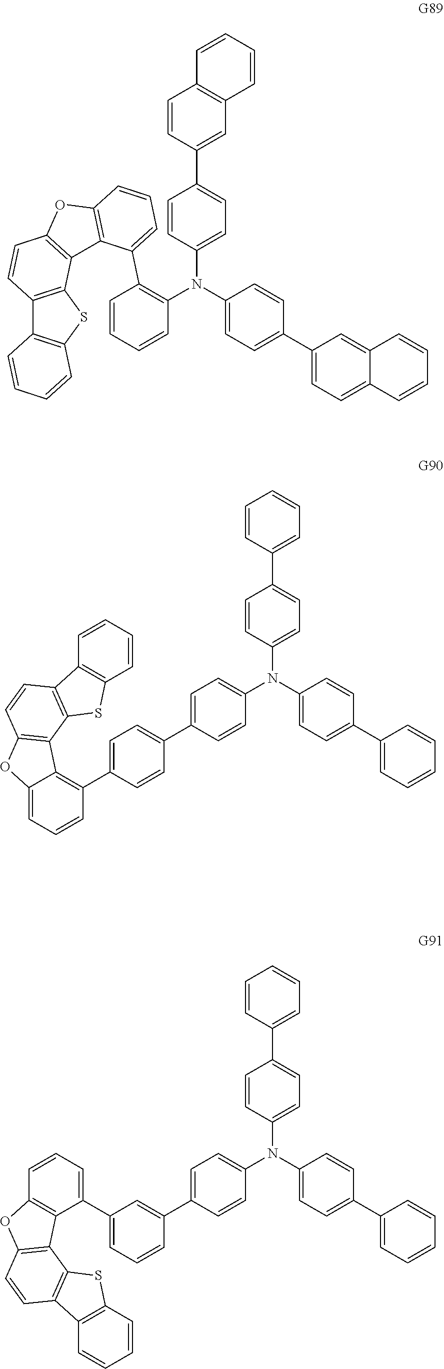 [SMILES: c1ccc(N(c2ccc(-c3ccc4ccccc4c3)cc2)c2ccc(-c3ccc4ccccc4c3)cc2)c(-c2cccc3oc4ccc5c6ccccc6sc5c4c23)c1 ; c1ccc(-c2ccc(N(c3ccc(-c4ccccc4)cc3)c3ccc(-c4ccc(-c5cccc6oc7ccc8c9ccccc9sc8c7c56)cc4)cc3)cc2)cc1 ; c1ccc(-c2ccc(N(c3ccc(-c4ccccc4)cc3)c3ccc(-c4cccc(-c5cccc6oc7ccc8c9ccccc9sc8c7c56)c4)cc3)cc2)cc1]

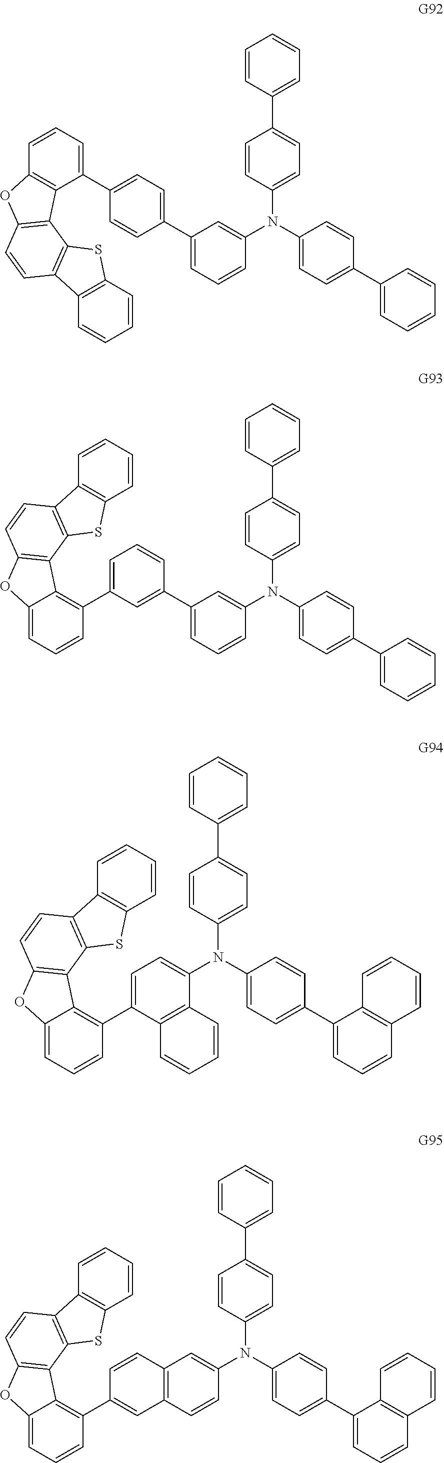 [SMILES: c1ccc(-c2ccc(N(c3ccc(-c4ccccc4)cc3)c3cccc(-c4ccc(-c5cccc6oc7ccc8c9ccccc9sc8c7c56)cc4)c3)cc2)cc1 ; c1ccc(-c2ccc(N(c3ccc(-c4ccccc4)cc3)c3cccc(-c4cccc(-c5cccc6oc7ccc8c9ccccc9sc8c7c56)c4)c3)cc2)cc1 ; c1ccc(-c2ccc(N(c3ccc(-c4cccc5ccccc45)cc3)c3ccc(-c4cccc5oc6ccc7c8ccccc8sc7c6c45)c4ccccc34)cc2)cc1 ; c1ccc(-c2ccc(N(c3ccc(-c4cccc5ccccc45)cc3)c3ccc4cc(-c5cccc6oc7ccc8c9ccccc9sc8c7c56)ccc4c3)cc2)cc1]

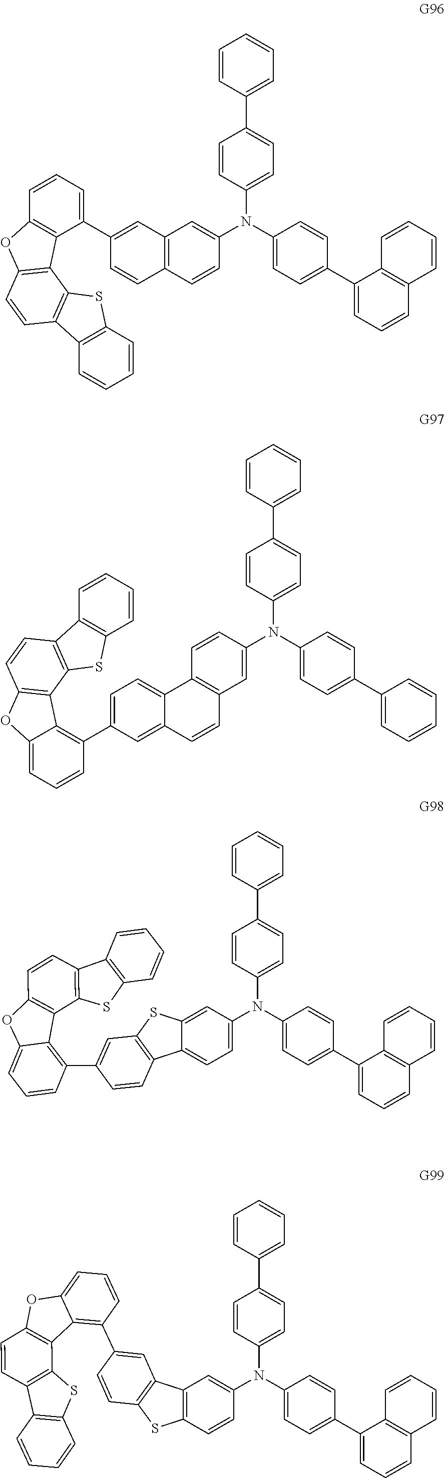 [SMILES: c1ccc(-c2ccc(N(c3ccc(-c4cccc5ccccc45)cc3)c3ccc4ccc(-c5cccc6oc7ccc8c9ccccc9sc8c7c56)cc4c3)cc2)cc1 ; c1ccc(-c2ccc(N(c3ccc(-c4ccccc4)cc3)c3ccc4c(ccc5cc(-c6cccc7oc8ccc9c%10ccccc%10sc9c8c67)ccc54)c3)cc2)cc1 ; c1ccc(-c2ccc(N(c3ccc(-c4cccc5ccccc45)cc3)c3ccc4c(c3)sc3cc(-c5cccc6oc7ccc8c9ccccc9sc8c7c56)ccc34)cc2)cc1 ; c1ccc(-c2ccc(N(c3ccc(-c4cccc5ccccc45)cc3)c3ccc4sc5ccc(-c6cccc7oc8ccc9c%10ccccc%10sc9c8c67)cc5c4c3)cc2)cc1]

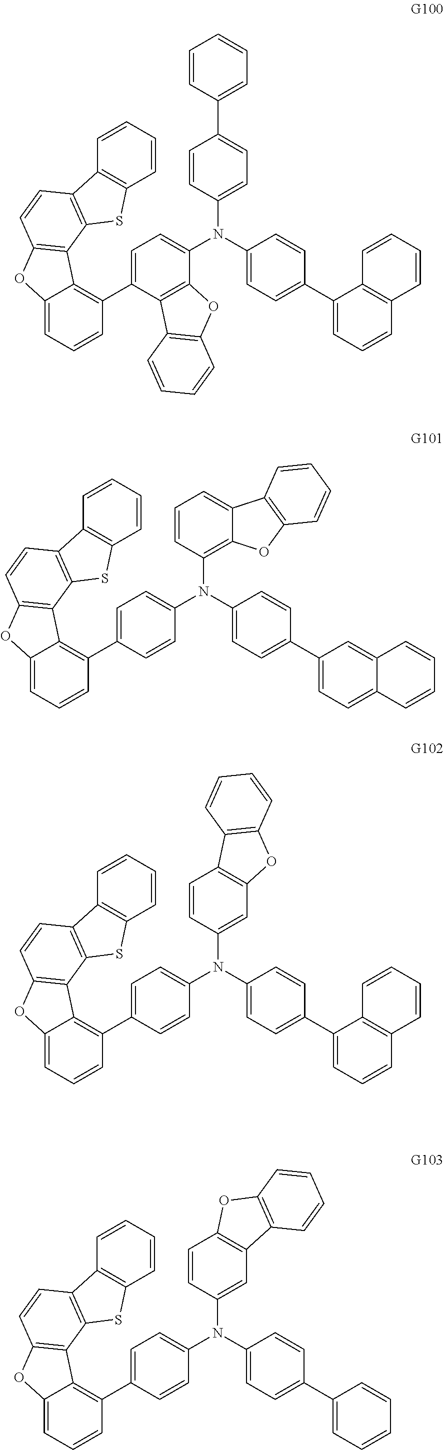 [SMILES: c1ccc(-c2ccc(N(c3ccc(-c4cccc5ccccc45)cc3)c3ccc(-c4cccc5oc6ccc7c8ccccc8sc7c6c45)c4c3oc3ccccc34)cc2)cc1 ; c1ccc2cc(-c3ccc(N(c4ccc(-c5cccc6oc7ccc8c9ccccc9sc8c7c56)cc4)c4cccc5c4oc4ccccc45)cc3)ccc2c1 ; c1ccc2c(-c3ccc(N(c4ccc(-c5cccc6oc7ccc8c9ccccc9sc8c7c56)cc4)c4ccc5c(c4)oc4ccccc45)cc3)cccc2c1 ; c1ccc(-c2ccc(N(c3ccc(-c4cccc5oc6ccc7c8ccccc8sc7c6c45)cc3)c3ccc4oc5ccccc5c4c3)cc2)cc1]

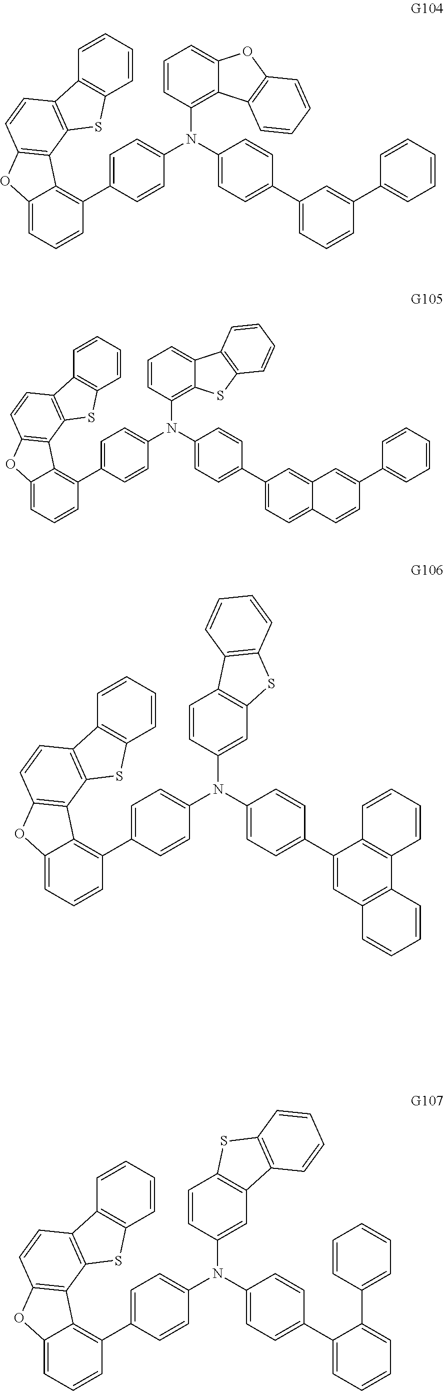 [SMILES: c1ccc(-c2cccc(-c3ccc(N(c4ccc(-c5cccc6oc7ccc8c9ccccc9sc8c7c56)cc4)c4cccc5oc6ccccc6c45)cc3)c2)cc1 ; c1ccc(-c2ccc3ccc(-c4ccc(N(c5ccc(-c6cccc7oc8ccc9c%10ccccc%10sc9c8c67)cc5)c5cccc6c5sc5ccccc56)cc4)cc3c2)cc1 ; c1ccc2c(c1)cc(-c1ccc(N(c3ccc(-c4cccc5oc6ccc7c8ccccc8sc7c6c45)cc3)c3ccc4c(c3)sc3ccccc34)cc1)c1ccccc12 ; c1ccc(-c2ccccc2-c2ccc(N(c3ccc(-c4cccc5oc6ccc7c8ccccc8sc7c6c45)cc3)c3ccc4sc5ccccc5c4c3)cc2)cc1]

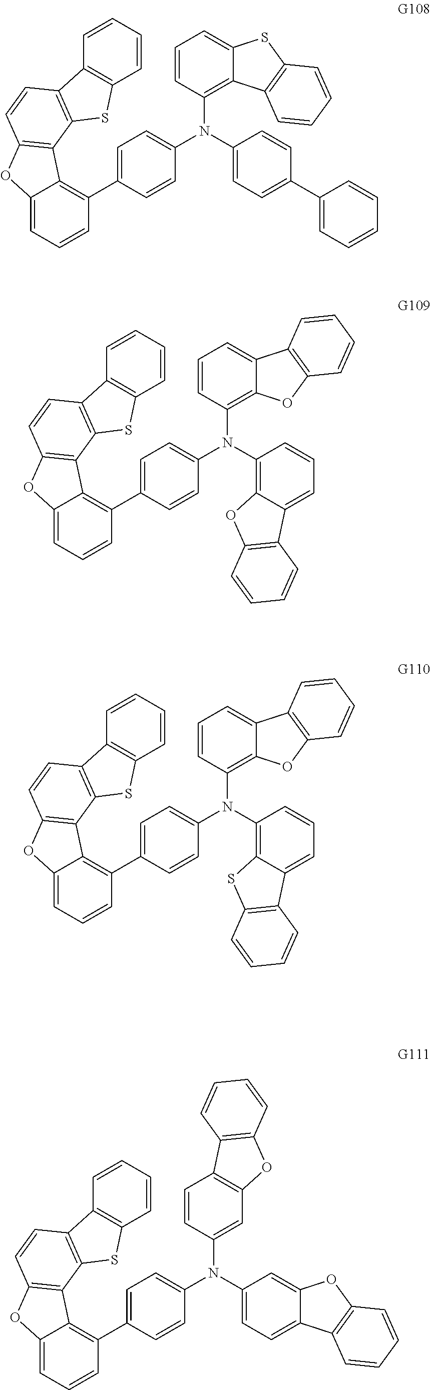 [SMILES: c1ccc(-c2ccc(N(c3ccc(-c4cccc5oc6ccc7c8ccccc8sc7c6c45)cc3)c3cccc4sc5ccccc5c34)cc2)cc1 ; c1ccc2c(c1)oc1c(N(c3ccc(-c4cccc5oc6ccc7c8ccccc8sc7c6c45)cc3)c3cccc4c3oc3ccccc34)cccc12 ; c1ccc2c(c1)oc1c(N(c3ccc(-c4cccc5oc6ccc7c8ccccc8sc7c6c45)cc3)c3cccc4c3sc3ccccc34)cccc12 ; c1ccc2c(c1)oc1cc(N(c3ccc(-c4cccc5oc6ccc7c8ccccc8sc7c6c45)cc3)c3ccc4c(c3)oc3ccccc34)ccc12]

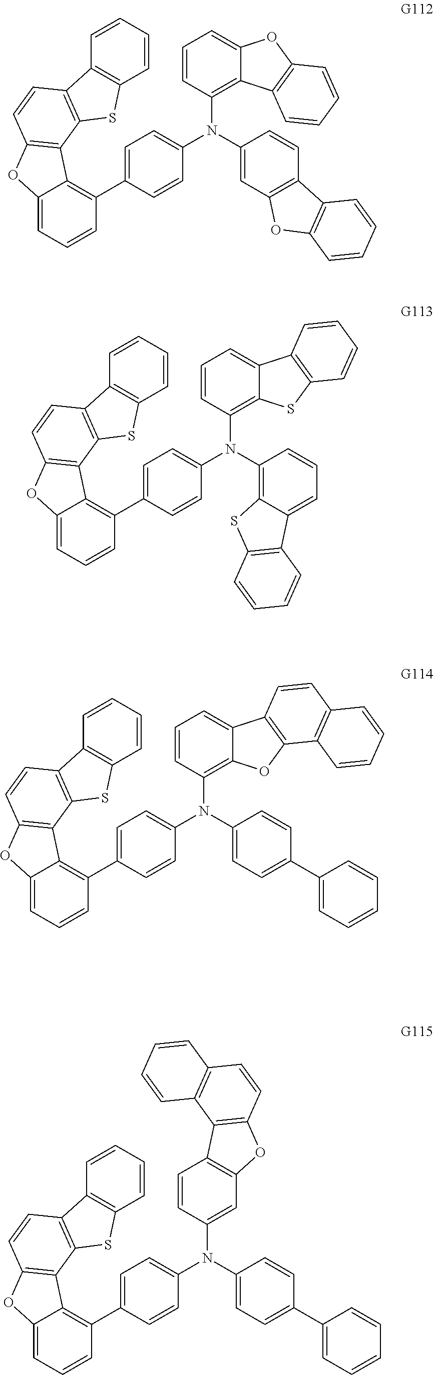 [SMILES: c1ccc2c(c1)oc1cc(N(c3ccc(-c4cccc5oc6ccc7c8ccccc8sc7c6c45)cc3)c3cccc4oc5ccccc5c34)ccc12 ; c1cc(-c2ccc(N(c3cccc4c3sc3ccccc34)c3cccc4c3sc3ccccc34)cc2)c2c(c1)oc1ccc3c4ccccc4sc3c12 ; c1ccc(-c2ccc(N(c3ccc(-c4cccc5oc6ccc7c8ccccc8sc7c6c45)cc3)c3cccc4c3oc3c5ccccc5ccc43)cc2)cc1 ; c1ccc(-c2ccc(N(c3ccc(-c4cccc5oc6ccc7c8ccccc8sc7c6c45)cc3)c3ccc4c(c3)oc3ccc5ccccc5c34)cc2)cc1]

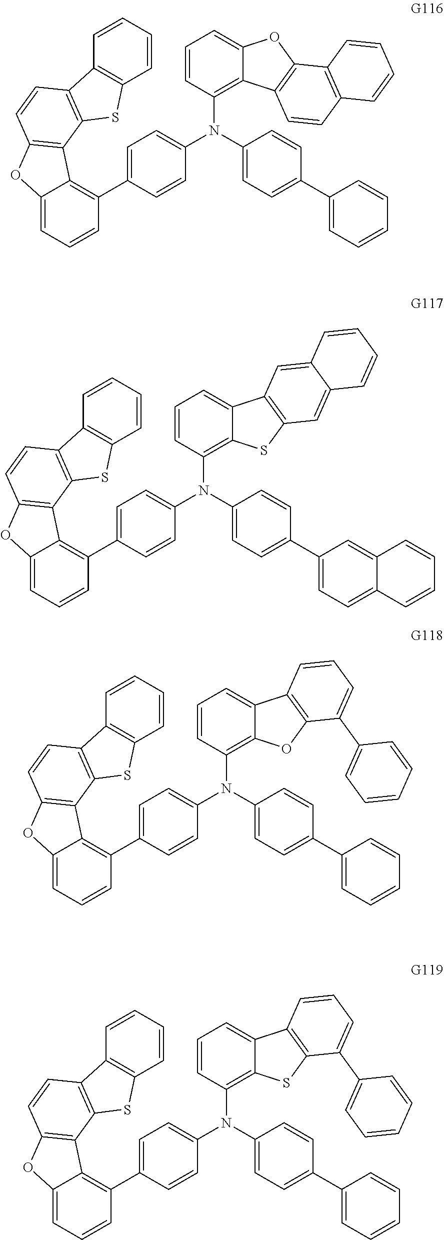 [SMILES: c1ccc(-c2ccc(N(c3ccc(-c4cccc5oc6ccc7c8ccccc8sc7c6c45)cc3)c3cccc4oc5c6ccccc6ccc5c34)cc2)cc1 ; c1ccc2cc(-c3ccc(N(c4ccc(-c5cccc6oc7ccc8c9ccccc9sc8c7c56)cc4)c4cccc5c4sc4cc6ccccc6cc45)cc3)ccc2c1 ; c1ccc(-c2ccc(N(c3ccc(-c4cccc5oc6ccc7c8ccccc8sc7c6c45)cc3)c3cccc4c3oc3c(-c5ccccc5)cccc34)cc2)cc1 ; c1ccc(-c2ccc(N(c3ccc(-c4cccc5oc6ccc7c8ccccc8sc7c6c45)cc3)c3cccc4c3sc3c(-c5ccccc5)cccc34)cc2)cc1]

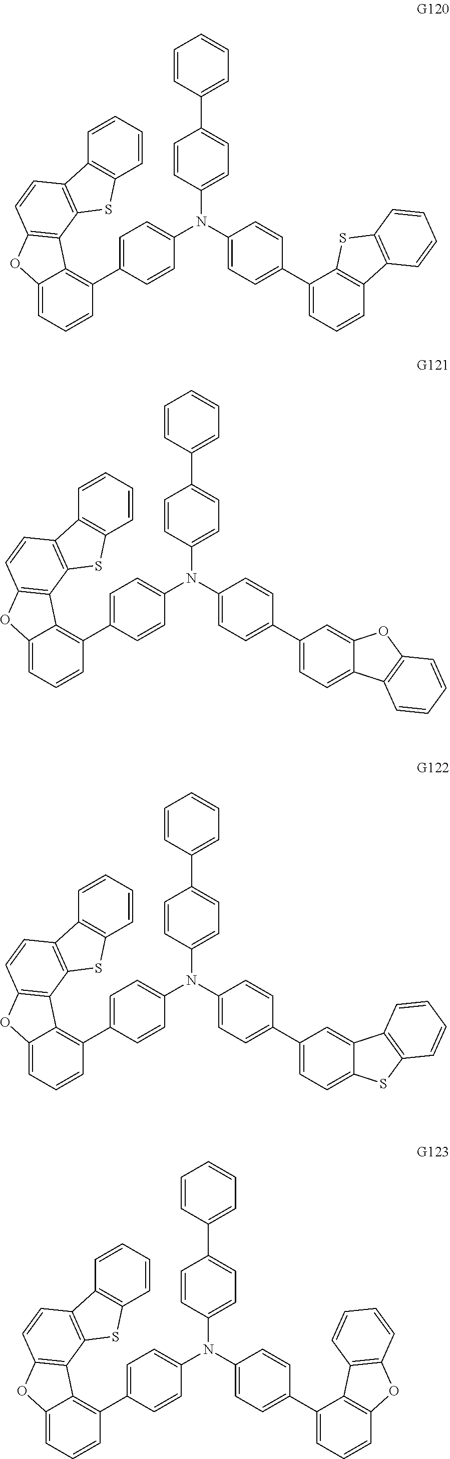 [SMILES: c1ccc(-c2ccc(N(c3ccc(-c4cccc5c4sc4ccccc45)cc3)c3ccc(-c4cccc5oc6ccc7c8ccccc8sc7c6c45)cc3)cc2)cc1 ; c1ccc(-c2ccc(N(c3ccc(-c4ccc5c(c4)oc4ccccc45)cc3)c3ccc(-c4cccc5oc6ccc7c8ccccc8sc7c6c45)cc3)cc2)cc1 ; c1ccc(-c2ccc(N(c3ccc(-c4ccc5sc6ccccc6c5c4)cc3)c3ccc(-c4cccc5oc6ccc7c8ccccc8sc7c6c45)cc3)cc2)cc1 ; c1ccc(-c2ccc(N(c3ccc(-c4cccc5oc6ccccc6c45)cc3)c3ccc(-c4cccc5oc6ccc7c8ccccc8sc7c6c45)cc3)cc2)cc1]

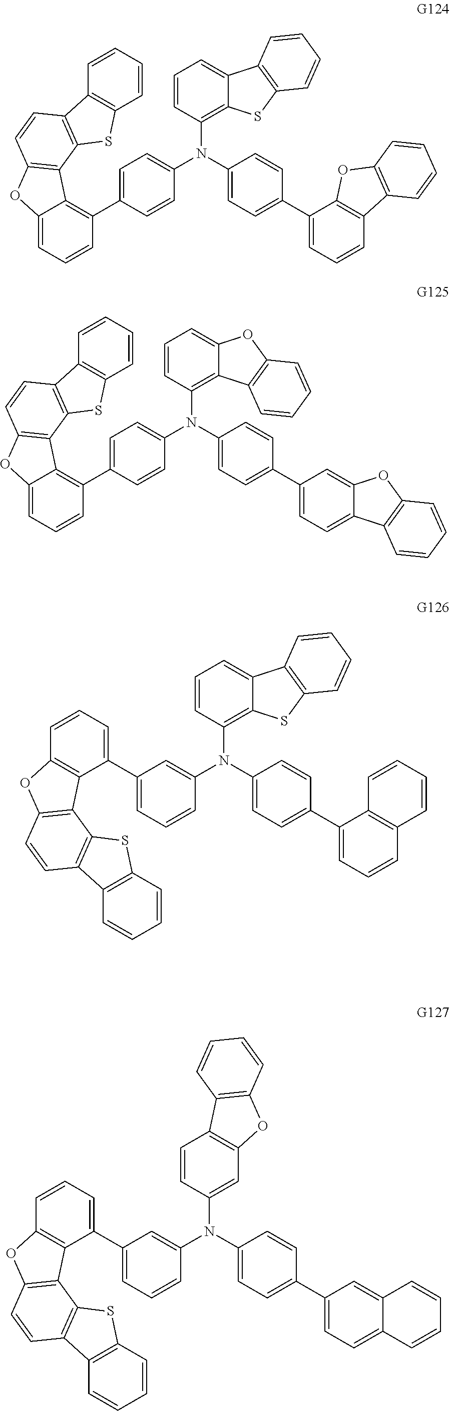 [SMILES: c1ccc2c(c1)oc1c(-c3ccc(N(c4ccc(-c5cccc6oc7ccc8c9ccccc9sc8c7c56)cc4)c4cccc5c4sc4ccccc45)cc3)cccc12 ; c1ccc2c(c1)oc1cc(-c3ccc(N(c4ccc(-c5cccc6oc7ccc8c9ccccc9sc8c7c56)cc4)c4cccc5oc6ccccc6c45)cc3)ccc12 ; c1cc(-c2cccc3oc4ccc5c6ccccc6sc5c4c23)cc(N(c2ccc(-c3cccc4ccccc34)cc2)c2cccc3c2sc2ccccc23)c1 ; c1cc(-c2cccc3oc4ccc5c6ccccc6sc5c4c23)cc(N(c2ccc(-c3ccc4ccccc4c3)cc2)c2ccc3c(c2)oc2ccccc23)c1]

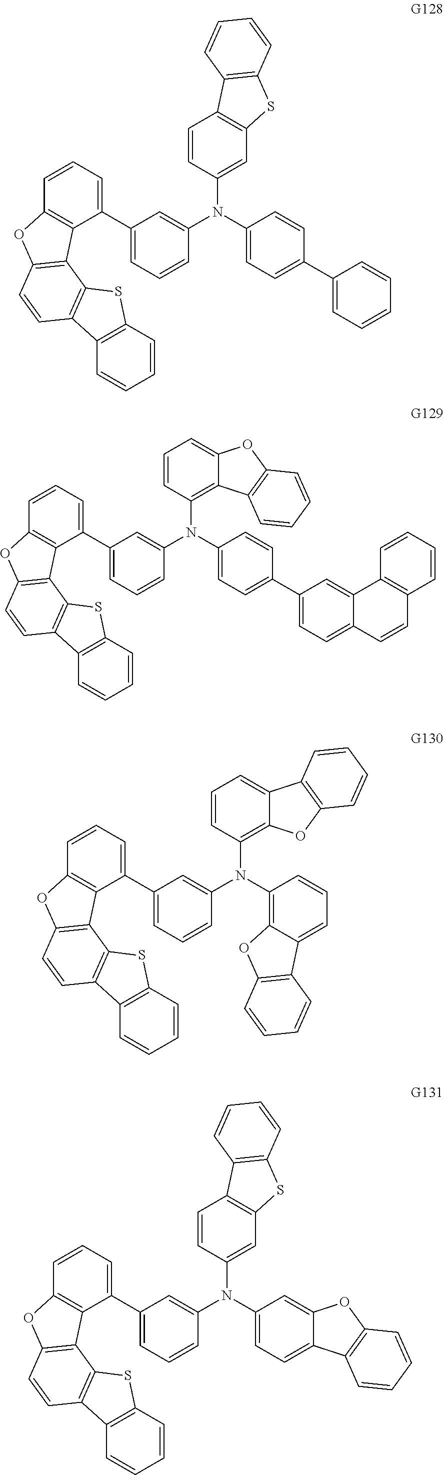 [SMILES: c1ccc(-c2ccc(N(c3cccc(-c4cccc5oc6ccc7c8ccccc8sc7c6c45)c3)c3ccc4c(c3)sc3ccccc34)cc2)cc1 ; c1cc(-c2cccc3oc4ccc5c6ccccc6sc5c4c23)cc(N(c2ccc(-c3ccc4ccc5ccccc5c4c3)cc2)c2cccc3oc4ccccc4c23)c1 ; c1cc(-c2cccc3oc4ccc5c6ccccc6sc5c4c23)cc(N(c2cccc3c2oc2ccccc23)c2cccc3c2oc2ccccc23)c1 ; c1cc(-c2cccc3oc4ccc5c6ccccc6sc5c4c23)cc(N(c2ccc3c(c2)oc2ccccc23)c2ccc3c(c2)sc2ccccc23)c1]

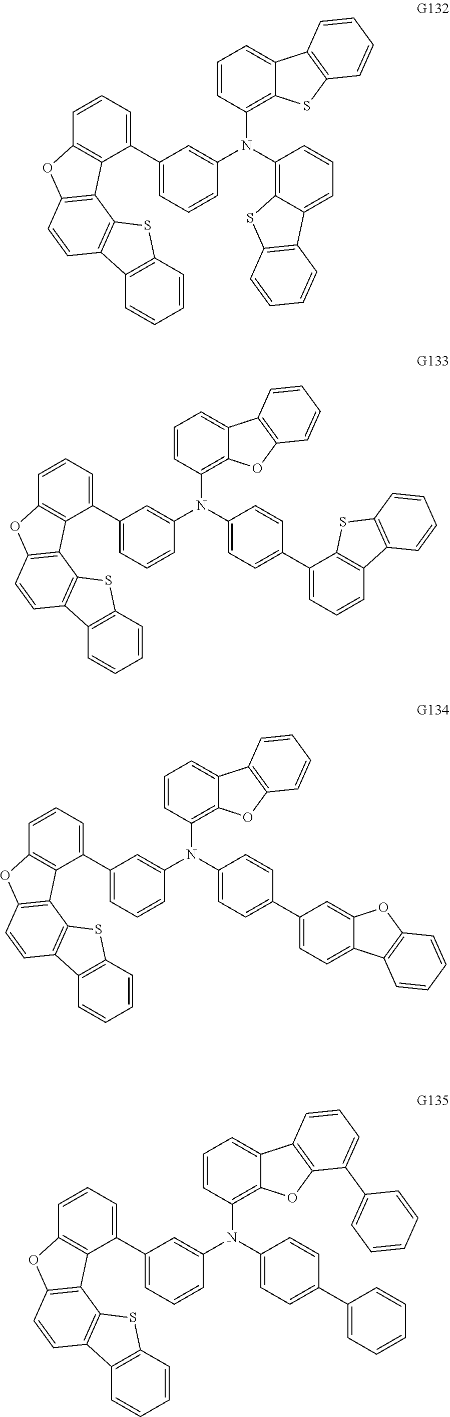 [SMILES: c1cc(-c2cccc3oc4ccc5c6ccccc6sc5c4c23)cc(N(c2cccc3c2sc2ccccc23)c2cccc3c2sc2ccccc23)c1 ; c1cc(-c2cccc3oc4ccc5c6ccccc6sc5c4c23)cc(N(c2ccc(-c3cccc4c3sc3ccccc34)cc2)c2cccc3c2oc2ccccc23)c1 ; c1cc(-c2cccc3oc4ccc5c6ccccc6sc5c4c23)cc(N(c2ccc(-c3ccc4c(c3)oc3ccccc34)cc2)c2cccc3c2oc2ccccc23)c1 ; c1ccc(-c2ccc(N(c3cccc(-c4cccc5oc6ccc7c8ccccc8sc7c6c45)c3)c3cccc4c3oc3c(-c5ccccc5)cccc34)cc2)cc1]

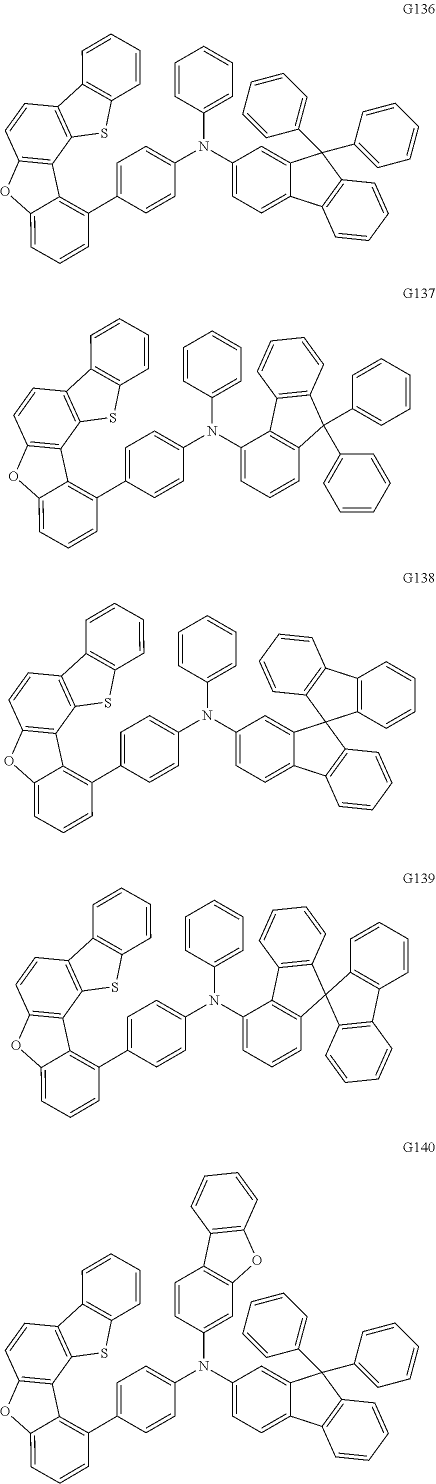 [SMILES: c1ccc(N(c2ccc(-c3cccc4oc5ccc6c7ccccc7sc6c5c34)cc2)c2ccc3c(c2)C(c2ccccc2)(c2ccccc2)c2ccccc2-3)cc1 ; c1ccc(N(c2ccc(-c3cccc4oc5ccc6c7ccccc7sc6c5c34)cc2)c2cccc3c2-c2ccccc2C3(c2ccccc2)c2ccccc2)cc1 ; c1ccc(N(c2ccc(-c3cccc4oc5ccc6c7ccccc7sc6c5c34)cc2)c2ccc3c(c2)C2(c4ccccc4-c4ccccc42)c2ccccc2-3)cc1 ; c1ccc(N(c2ccc(-c3cccc4oc5ccc6c7ccccc7sc6c5c34)cc2)c2cccc3c2-c2ccccc2C32c3ccccc3-c3ccccc32)cc1 ; c1ccc(C2(c3ccccc3)c3ccccc3-c3ccc(N(c4ccc(-c5cccc6oc7ccc8c9ccccc9sc8c7c56)cc4)c4ccc5c(c4)oc4ccccc45)cc32)cc1]

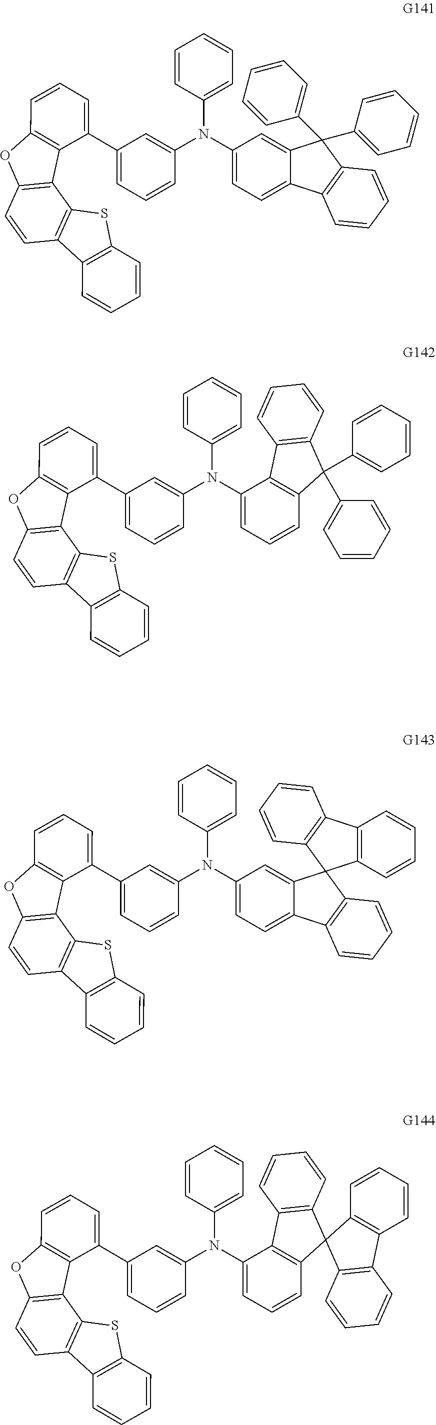 [SMILES: c1ccc(N(c2cccc(-c3cccc4oc5ccc6c7ccccc7sc6c5c34)c2)c2ccc3c(c2)C(c2ccccc2)(c2ccccc2)c2ccccc2-3)cc1 ; c1ccc(N(c2cccc(-c3cccc4oc5ccc6c7ccccc7sc6c5c34)c2)c2cccc3c2-c2ccccc2C3(c2ccccc2)c2ccccc2)cc1 ; c1ccc(N(c2cccc(-c3cccc4oc5ccc6c7ccccc7sc6c5c34)c2)c2ccc3c(c2)C2(c4ccccc4-c4ccccc42)c2ccccc2-3)cc1 ; c1ccc(N(c2cccc(-c3cccc4oc5ccc6c7ccccc7sc6c5c34)c2)c2cccc3c2-c2ccccc2C32c3ccccc3-c3ccccc32)cc1]

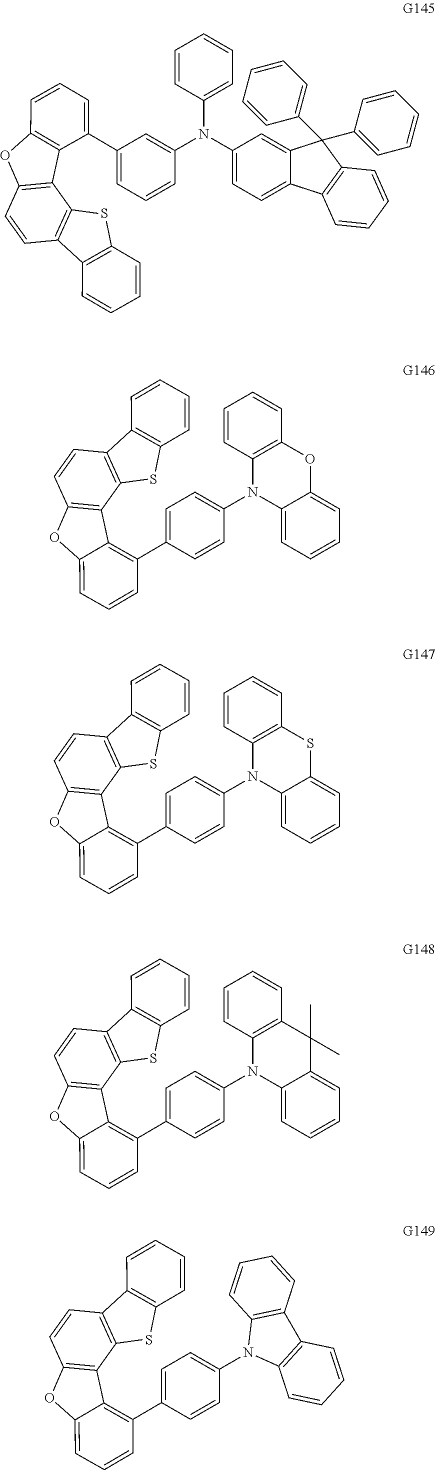 [SMILES: c1ccc(N(c2cccc(-c3cccc4oc5ccc6c7ccccc7sc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3oc4ccc5c6ccccc6sc5c4c23)cc1 ; c1ccc2c(c1)Sc1ccccc1N2c1ccc(-c2cccc3oc4ccc5c6ccccc6sc5c4c23)cc1 ; CC1(C)c2ccccc2N(c2ccc(-c3cccc4oc5ccc6c7ccccc7sc6c5c34)cc2)c2ccccc21 ; c1cc(-c2ccc(-n3c4ccccc4c4ccccc43)cc2)c2c(c1)oc1ccc3c4ccccc4sc3c12]

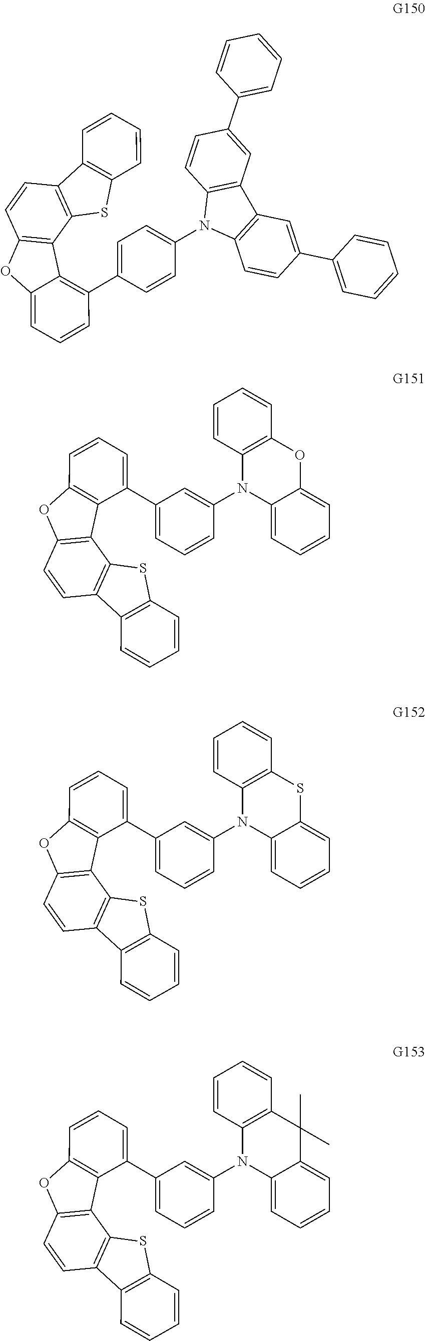 [SMILES: c1ccc(-c2ccc3c(c2)c2cc(-c4ccccc4)ccc2n3-c2ccc(-c3cccc4oc5ccc6c7ccccc7sc6c5c34)cc2)cc1 ; c1cc(-c2cccc3oc4ccc5c6ccccc6sc5c4c23)cc(N2c3ccccc3Oc3ccccc32)c1 ; c1cc(-c2cccc3oc4ccc5c6ccccc6sc5c4c23)cc(N2c3ccccc3Sc3ccccc32)c1 ; CC1(C)c2ccccc2N(c2cccc(-c3cccc4oc5ccc6c7ccccc7sc6c5c34)c2)c2ccccc21]

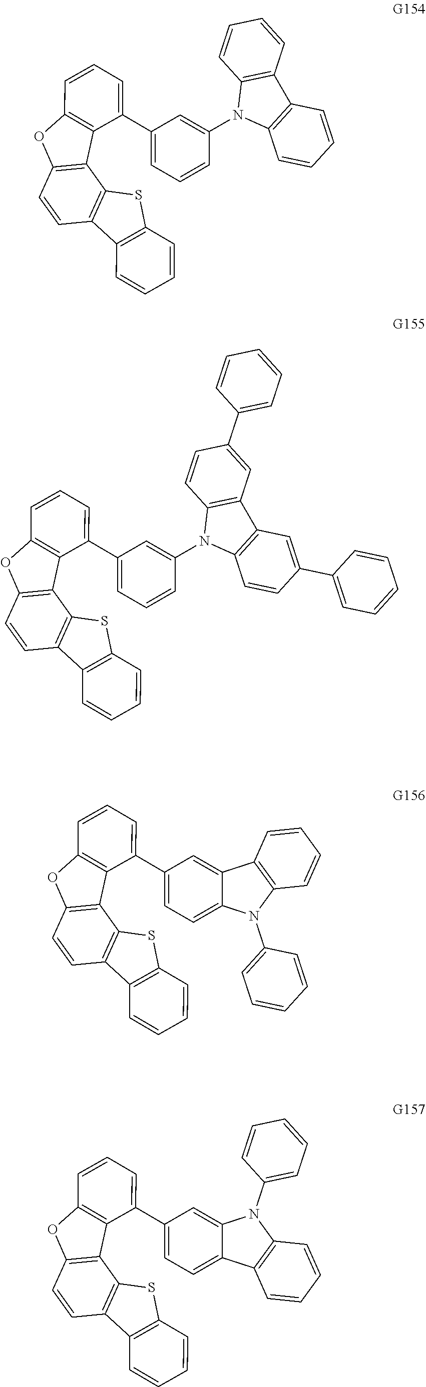 [SMILES: c1cc(-c2cccc3oc4ccc5c6ccccc6sc5c4c23)cc(-n2c3ccccc3c3ccccc32)c1 ; c1ccc(-c2ccc3c(c2)c2cc(-c4ccccc4)ccc2n3-c2cccc(-c3cccc4oc5ccc6c7ccccc7sc6c5c34)c2)cc1 ; c1ccc(-n2c3ccccc3c3cc(-c4cccc5oc6ccc7c8ccccc8sc7c6c45)ccc32)cc1 ; c1ccc(-n2c3ccccc3c3ccc(-c4cccc5oc6ccc7c8ccccc8sc7c6c45)cc32)cc1]

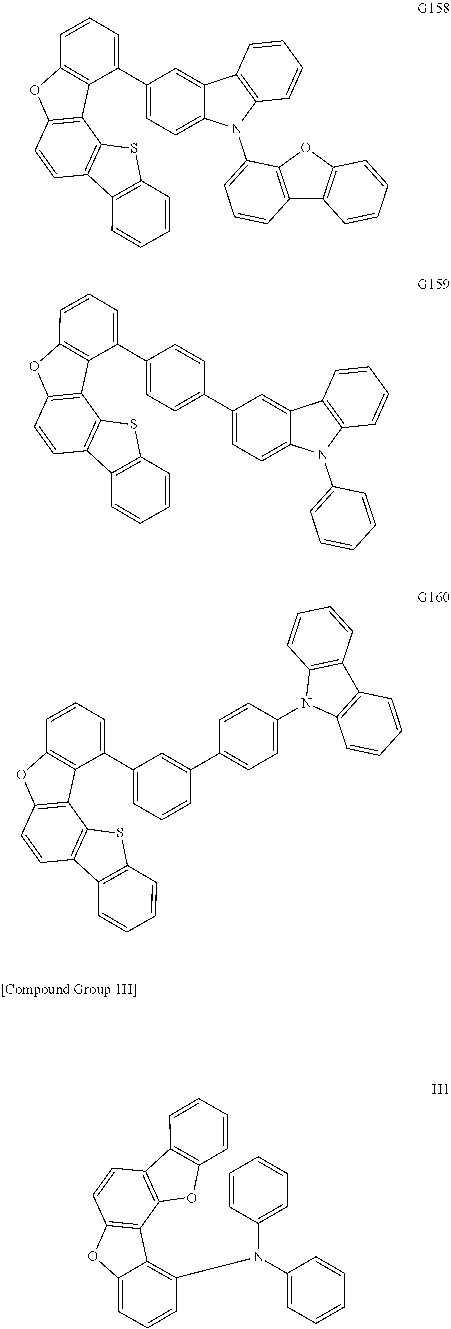 [SMILES: c1ccc2c(c1)oc1c(-n3c4ccccc4c4cc(-c5cccc6oc7ccc8c9ccccc9sc8c7c56)ccc43)cccc12 ; c1ccc(-n2c3ccccc3c3cc(-c4ccc(-c5cccc6oc7ccc8c9ccccc9sc8c7c56)cc4)ccc32)cc1 ; c1cc(-c2ccc(-n3c4ccccc4c4ccccc43)cc2)cc(-c2cccc3oc4ccc5c6ccccc6sc5c4c23)c1 ; c1ccc(N(c2ccccc2)c2cccc3oc4ccc5c6ccccc6oc5c4c23)cc1]

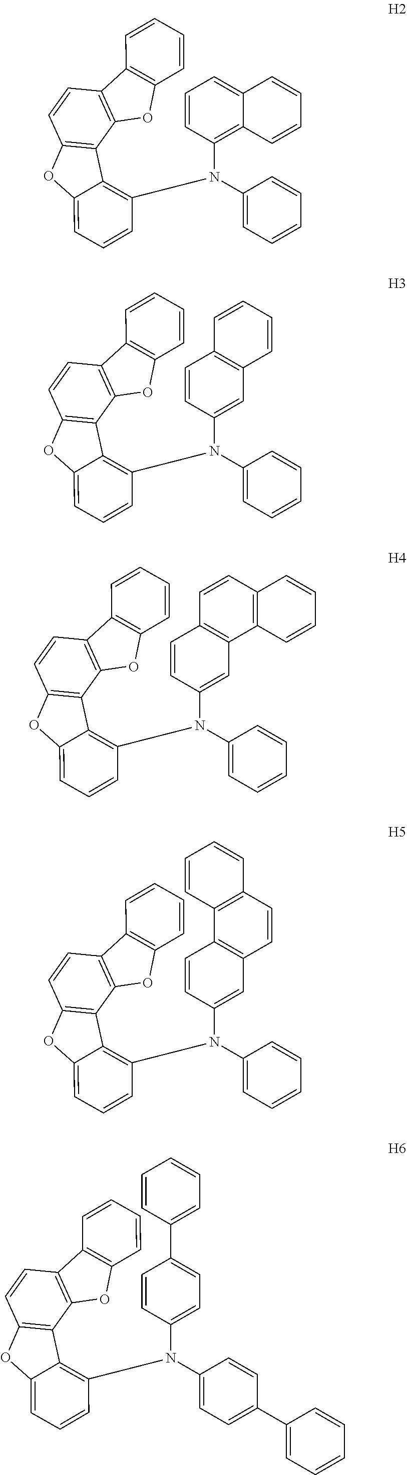 [SMILES: c1ccc(N(c2cccc3ccccc23)c2cccc3oc4ccc5c6ccccc6oc5c4c23)cc1 ; c1ccc(N(c2ccc3ccccc3c2)c2cccc3oc4ccc5c6ccccc6oc5c4c23)cc1 ; c1ccc(N(c2ccc3ccc4ccccc4c3c2)c2cccc3oc4ccc5c6ccccc6oc5c4c23)cc1 ; c1ccc(N(c2ccc3c(ccc4ccccc43)c2)c2cccc3oc4ccc5c6ccccc6oc5c4c23)cc1 ; c1ccc(-c2ccc(N(c3ccc(-c4ccccc4)cc3)c3cccc4oc5ccc6c7ccccc7oc6c5c34)cc2)cc1]

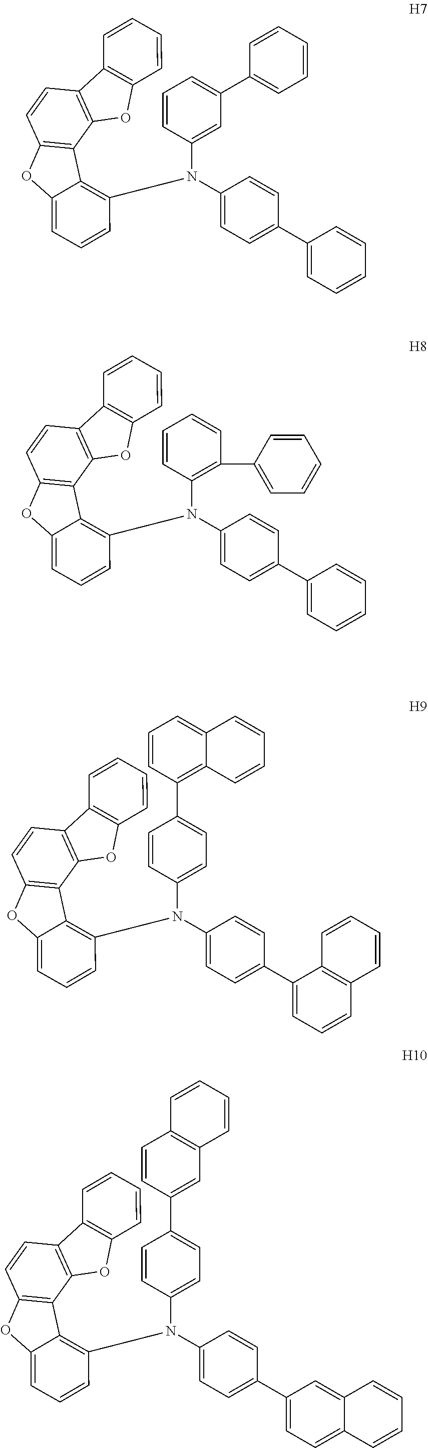 [SMILES: c1ccc(-c2ccc(N(c3cccc(-c4ccccc4)c3)c3cccc4oc5ccc6c7ccccc7oc6c5c34)cc2)cc1 ; c1ccc(-c2ccc(N(c3ccccc3-c3ccccc3)c3cccc4oc5ccc6c7ccccc7oc6c5c34)cc2)cc1 ; c1ccc2c(-c3ccc(N(c4ccc(-c5cccc6ccccc56)cc4)c4cccc5oc6ccc7c8ccccc8oc7c6c45)cc3)cccc2c1 ; c1ccc2cc(-c3ccc(N(c4ccc(-c5ccc6ccccc6c5)cc4)c4cccc5oc6ccc7c8ccccc8oc7c6c45)cc3)ccc2c1]

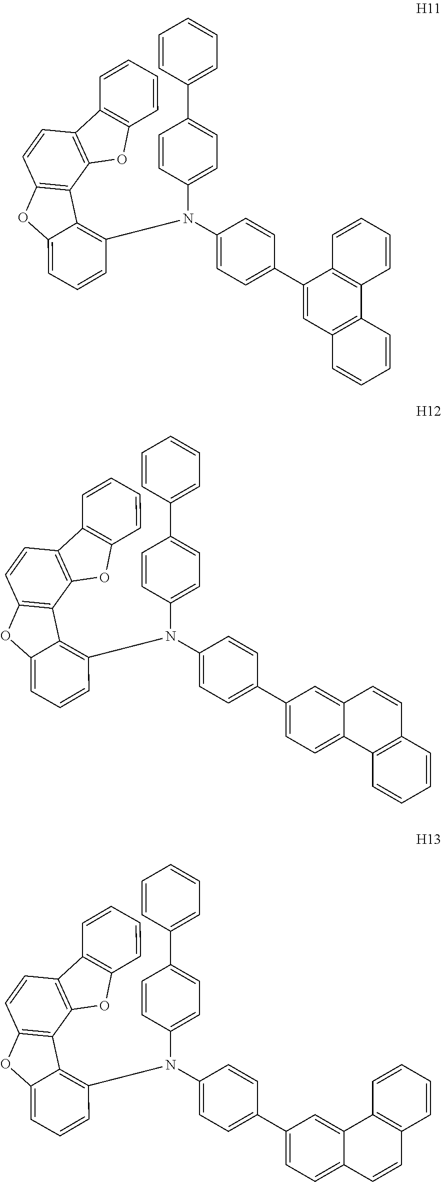 [SMILES: c1ccc(-c2ccc(N(c3ccc(-c4cc5ccccc5c5ccccc45)cc3)c3cccc4oc5ccc6c7ccccc7oc6c5c34)cc2)cc1 ; c1ccc(-c2ccc(N(c3ccc(-c4ccc5c(ccc6ccccc65)c4)cc3)c3cccc4oc5ccc6c7ccccc7oc6c5c34)cc2)cc1 ; c1ccc(-c2ccc(N(c3ccc(-c4ccc5ccc6ccccc6c5c4)cc3)c3cccc4oc5ccc6c7ccccc7oc6c5c34)cc2)cc1]

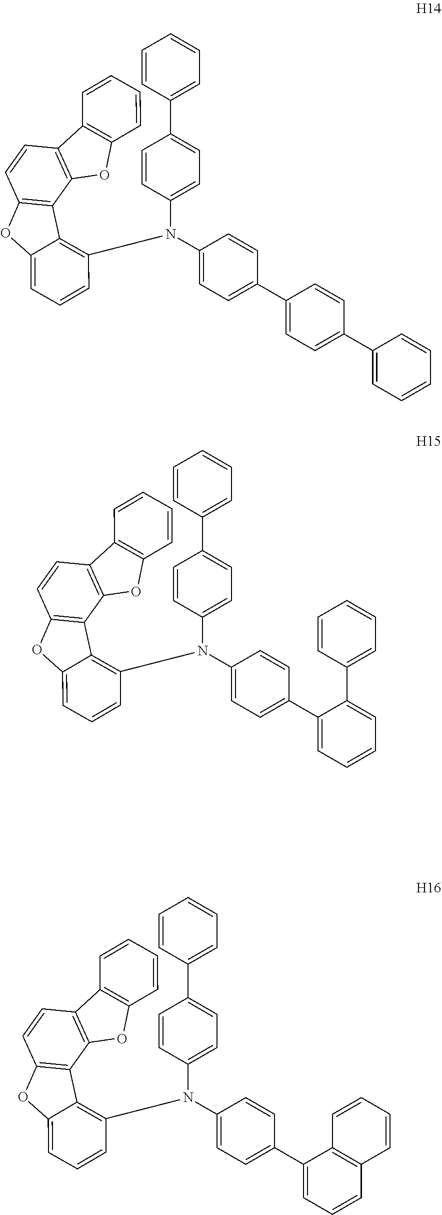 [SMILES: c1ccc(-c2ccc(-c3ccc(N(c4ccc(-c5ccccc5)cc4)c4cccc5oc6ccc7c8ccccc8oc7c6c45)cc3)cc2)cc1 ; c1ccc(-c2ccc(N(c3ccc(-c4ccccc4-c4ccccc4)cc3)c3cccc4oc5ccc6c7ccccc7oc6c5c34)cc2)cc1 ; c1ccc(-c2ccc(N(c3ccc(-c4cccc5ccccc45)cc3)c3cccc4oc5ccc6c7ccccc7oc6c5c34)cc2)cc1]

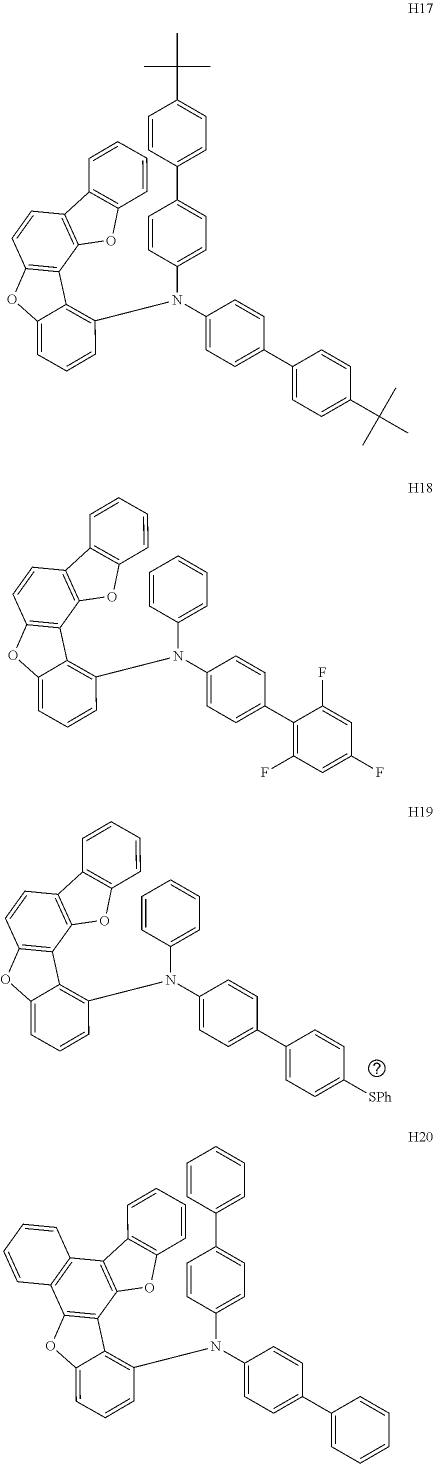

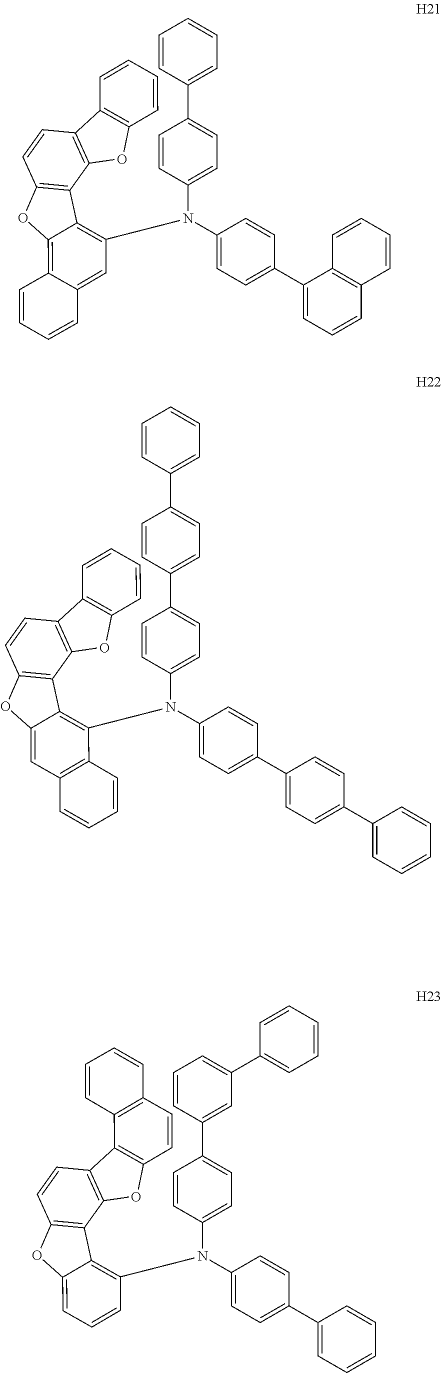 [SMILES: c1ccc(-c2ccc(N(c3ccc(-c4cccc5ccccc45)cc3)c3cc4ccccc4c4oc5ccc6c7ccccc7oc6c5c34)cc2)cc1 ; c1ccc(-c2ccc(-c3ccc(N(c4ccc(-c5ccc(-c6ccccc6)cc5)cc4)c4c5ccccc5cc5oc6ccc7c8ccccc8oc7c6c45)cc3)cc2)cc1 ; c1ccc(-c2ccc(N(c3ccc(-c4cccc(-c5ccccc5)c4)cc3)c3cccc4oc5ccc6c(oc7ccc8ccccc8c76)c5c34)cc2)cc1]

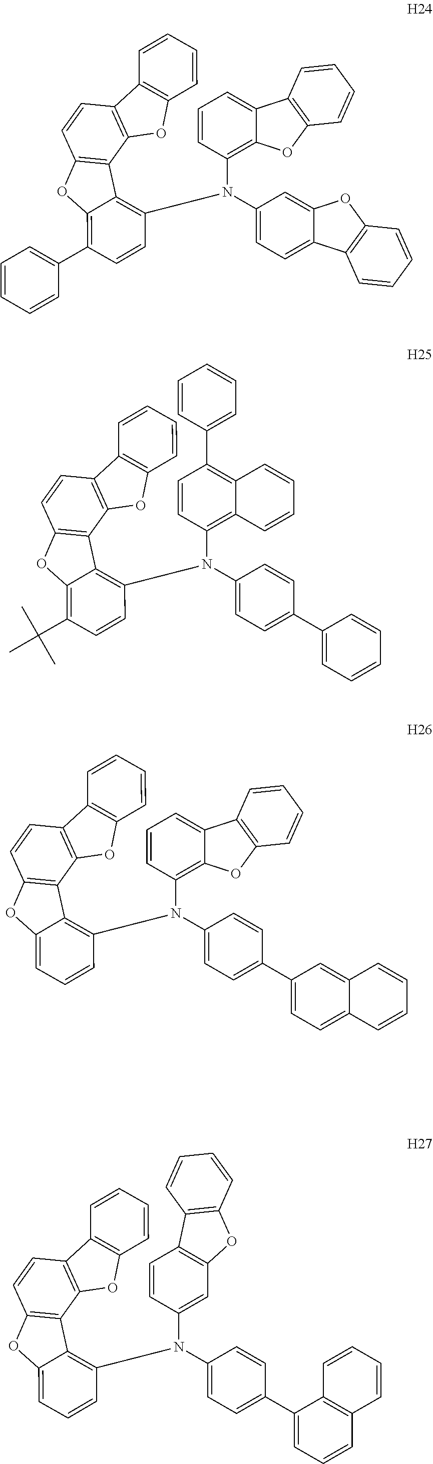 [SMILES: c1ccc(-c2ccc(N(c3ccc4c(c3)oc3ccccc34)c3cccc4c3oc3ccccc34)c3c2oc2ccc4c5ccccc5oc4c23)cc1 ; CC(C)(C)c1ccc(N(c2ccc(-c3ccccc3)cc2)c2ccc(-c3ccccc3)c3ccccc23)c2c1oc1ccc3c4ccccc4oc3c12 ; c1ccc2cc(-c3ccc(N(c4cccc5c4oc4ccccc45)c4cccc5oc6ccc7c8ccccc8oc7c6c45)cc3)ccc2c1 ; c1ccc2c(-c3ccc(N(c4ccc5c(c4)oc4ccccc45)c4cccc5oc6ccc7c8ccccc8oc7c6c45)cc3)cccc2c1]

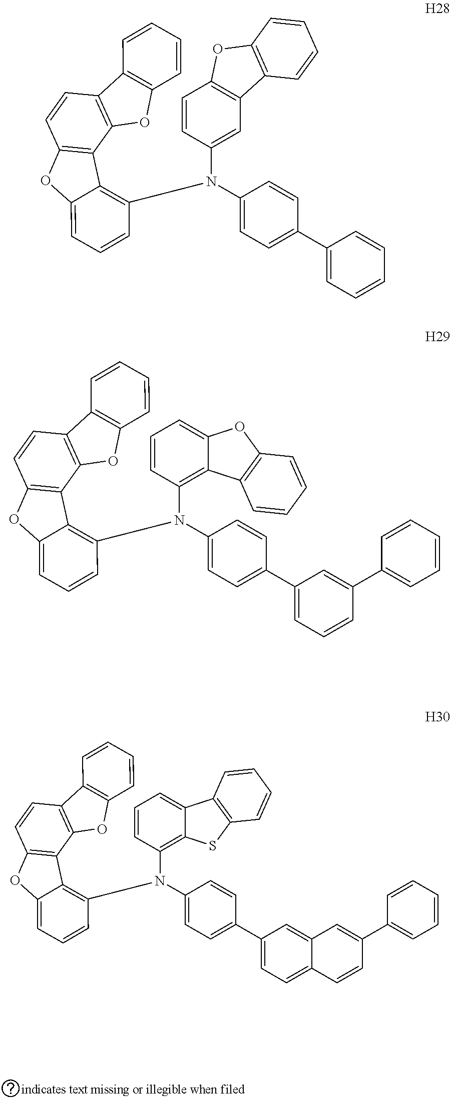 [SMILES: c1ccc(-c2ccc(N(c3ccc4oc5ccccc5c4c3)c3cccc4oc5ccc6c7ccccc7oc6c5c34)cc2)cc1 ; c1ccc(-c2cccc(-c3ccc(N(c4cccc5oc6ccccc6c45)c4cccc5oc6ccc7c8ccccc8oc7c6c45)cc3)c2)cc1 ; c1ccc(-c2ccc3ccc(-c4ccc(N(c5cccc6c5sc5ccccc56)c5cccc6oc7ccc8c9ccccc9oc8c7c56)cc4)cc3c2)cc1]

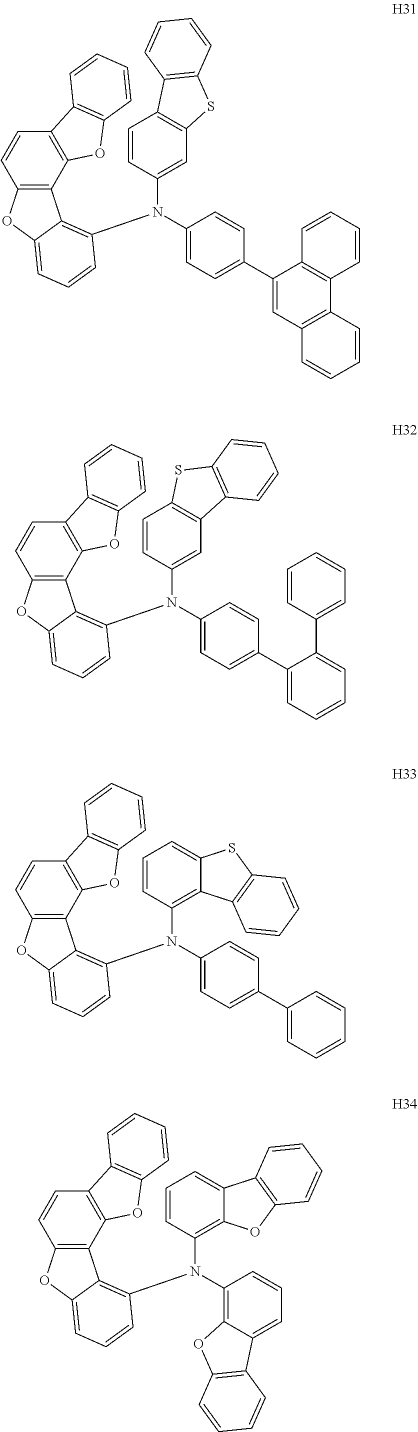 [SMILES: c1ccc2c(c1)cc(-c1ccc(N(c3ccc4c(c3)sc3ccccc34)c3cccc4oc5ccc6c7ccccc7oc6c5c34)cc1)c1ccccc12 ; c1ccc(-c2ccccc2-c2ccc(N(c3ccc4sc5ccccc5c4c3)c3cccc4oc5ccc6c7ccccc7oc6c5c34)cc2)cc1 ; c1ccc(-c2ccc(N(c3cccc4sc5ccccc5c34)c3cccc4oc5ccc6c7ccccc7oc6c5c34)cc2)cc1 ; c1ccc2c(c1)oc1c(N(c3cccc4c3oc3ccccc34)c3cccc4oc5ccc6c7ccccc7oc6c5c34)cccc12]

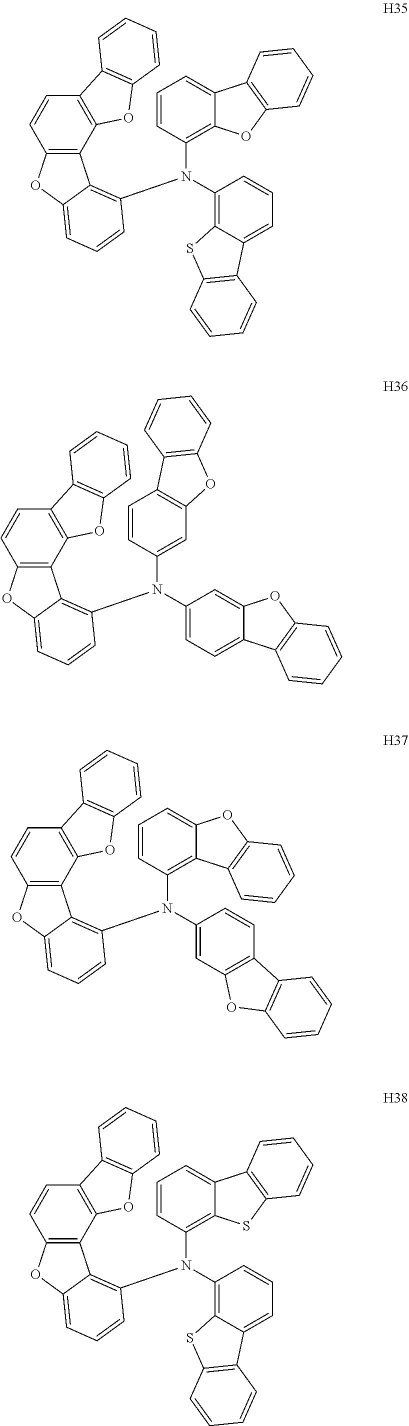 [SMILES: c1ccc2c(c1)oc1c(N(c3cccc4c3sc3ccccc34)c3cccc4oc5ccc6c7ccccc7oc6c5c34)cccc12 ; c1ccc2c(c1)oc1cc(N(c3ccc4c(c3)oc3ccccc34)c3cccc4oc5ccc6c7ccccc7oc6c5c34)ccc12 ; c1ccc2c(c1)oc1cc(N(c3cccc4oc5ccccc5c34)c3cccc4oc5ccc6c7ccccc7oc6c5c34)ccc12 ; c1ccc2c(c1)oc1c2ccc2oc3cccc(N(c4cccc5c4sc4ccccc45)c4cccc5c4sc4ccccc45)c3c21]

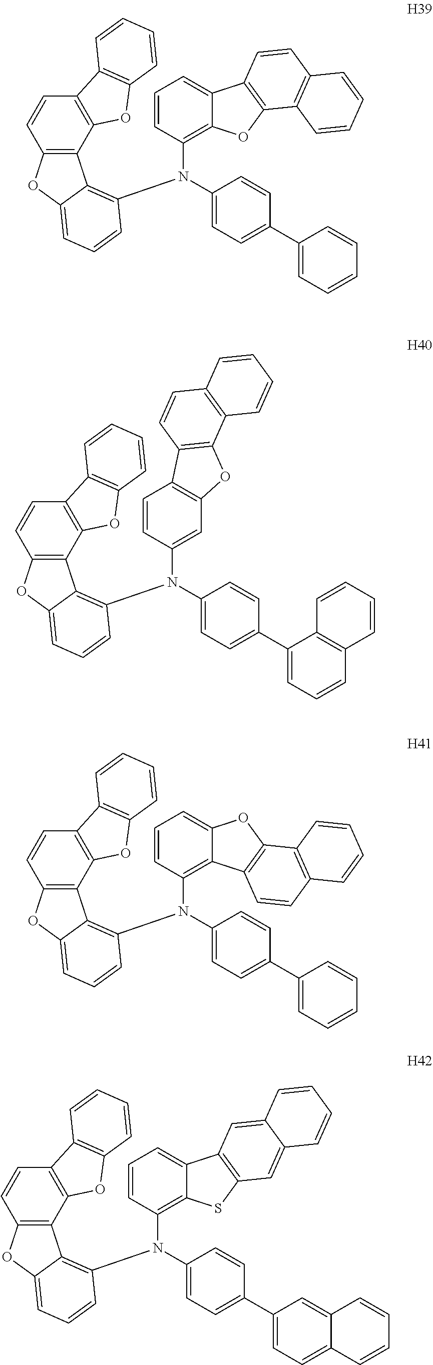 [SMILES: c1ccc(-c2ccc(N(c3cccc4c3oc3c5ccccc5ccc43)c3cccc4oc5ccc6c7ccccc7oc6c5c34)cc2)cc1 ; c1ccc2c(-c3ccc(N(c4ccc5c(c4)oc4c6ccccc6ccc54)c4cccc5oc6ccc7c8ccccc8oc7c6c45)cc3)cccc2c1 ; c1ccc(-c2ccc(N(c3cccc4oc5c6ccccc6ccc5c34)c3cccc4oc5ccc6c7ccccc7oc6c5c34)cc2)cc1 ; c1ccc2cc(-c3ccc(N(c4cccc5c4sc4cc6ccccc6cc45)c4cccc5oc6ccc7c8ccccc8oc7c6c45)cc3)ccc2c1]

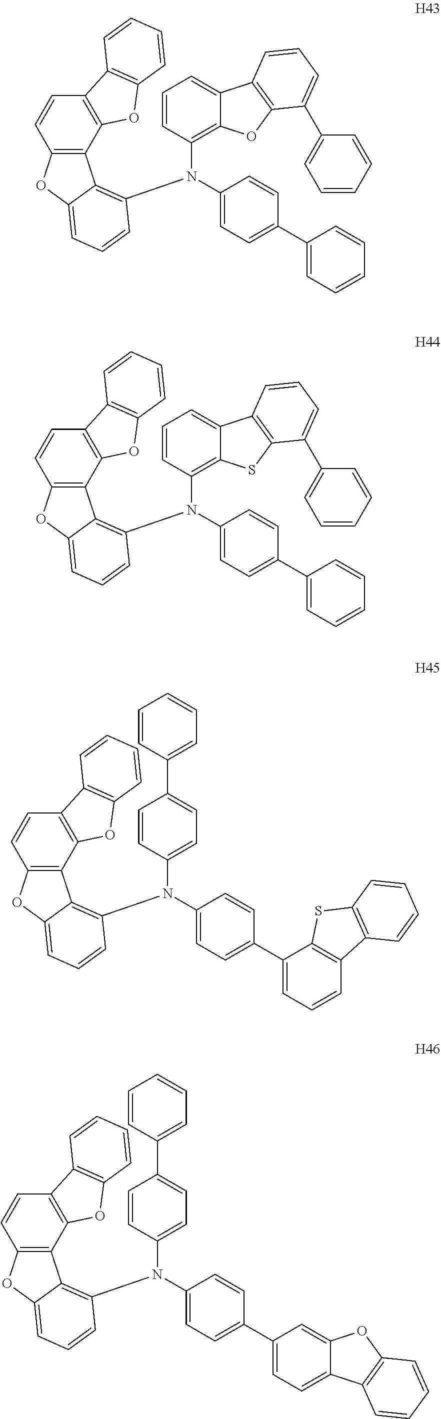 [SMILES: c1ccc(-c2ccc(N(c3cccc4c3oc3c(-c5ccccc5)cccc34)c3cccc4oc5ccc6c7ccccc7oc6c5c34)cc2)cc1 ; c1ccc(-c2ccc(N(c3cccc4c3sc3c(-c5ccccc5)cccc34)c3cccc4oc5ccc6c7ccccc7oc6c5c34)cc2)cc1 ; c1ccc(-c2ccc(N(c3ccc(-c4cccc5c4sc4ccccc45)cc3)c3cccc4oc5ccc6c7ccccc7oc6c5c34)cc2)cc1 ; c1ccc(-c2ccc(N(c3ccc(-c4ccc5c(c4)oc4ccccc45)cc3)c3cccc4oc5ccc6c7ccccc7oc6c5c34)cc2)cc1]

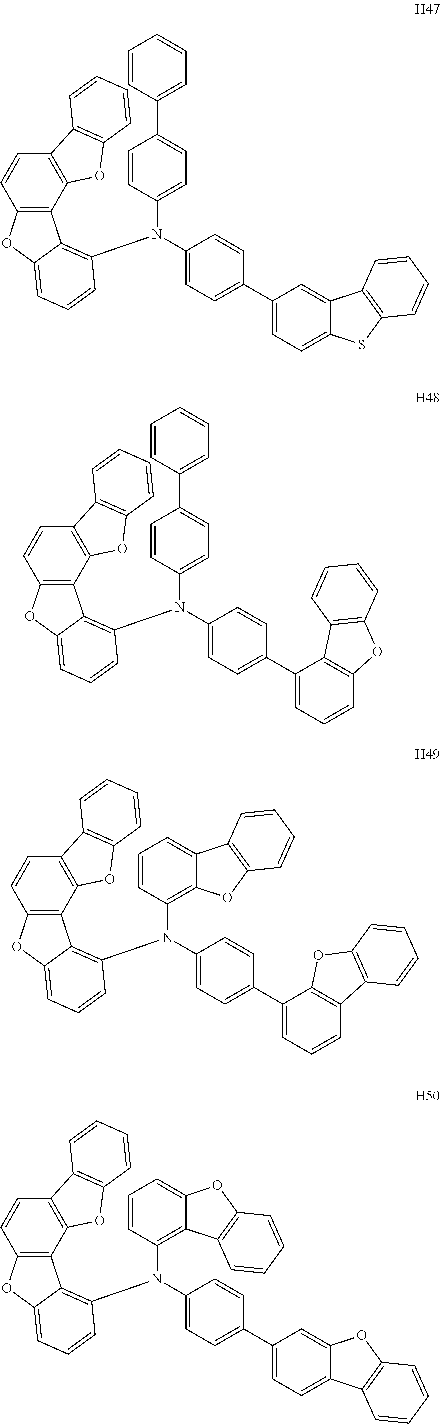 [SMILES: c1ccc(-c2ccc(N(c3ccc(-c4ccc5sc6ccccc6c5c4)cc3)c3cccc4oc5ccc6c7ccccc7oc6c5c34)cc2)cc1 ; c1ccc(-c2ccc(N(c3ccc(-c4cccc5oc6ccccc6c45)cc3)c3cccc4oc5ccc6c7ccccc7oc6c5c34)cc2)cc1 ; c1ccc2c(c1)oc1c(-c3ccc(N(c4cccc5c4oc4ccccc45)c4cccc5oc6ccc7c8ccccc8oc7c6c45)cc3)cccc12 ; c1ccc2c(c1)oc1cc(-c3ccc(N(c4cccc5oc6ccccc6c45)c4cccc5oc6ccc7c8ccccc8oc7c6c45)cc3)ccc12]

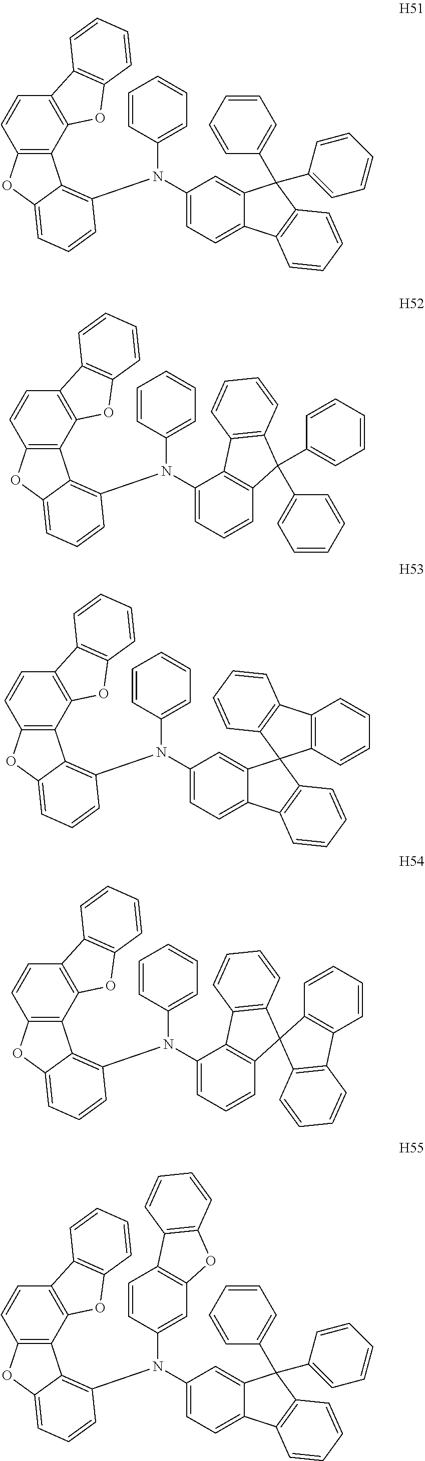 [SMILES: c1ccc(N(c2ccc3c(c2)C(c2ccccc2)(c2ccccc2)c2ccccc2-3)c2cccc3oc4ccc5c6ccccc6oc5c4c23)cc1 ; c1ccc(N(c2cccc3c2-c2ccccc2C3(c2ccccc2)c2ccccc2)c2cccc3oc4ccc5c6ccccc6oc5c4c23)cc1 ; c1ccc(N(c2ccc3c(c2)C2(c4ccccc4-c4ccccc42)c2ccccc2-3)c2cccc3oc4ccc5c6ccccc6oc5c4c23)cc1 ; c1ccc(N(c2cccc3c2-c2ccccc2C32c3ccccc3-c3ccccc32)c2cccc3oc4ccc5c6ccccc6oc5c4c23)cc1 ; c1ccc(C2(c3ccccc3)c3ccccc3-c3ccc(N(c4ccc5c(c4)oc4ccccc45)c4cccc5oc6ccc7c8ccccc8oc7c6c45)cc32)cc1]

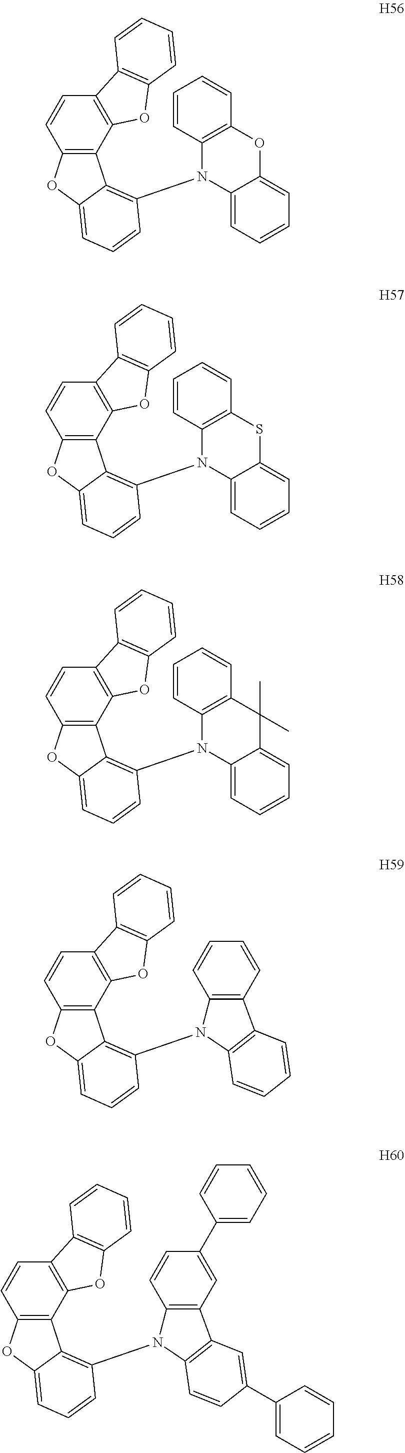 [SMILES: c1ccc2c(c1)Oc1ccccc1N2c1cccc2oc3ccc4c5ccccc5oc4c3c12 ; c1ccc2c(c1)Sc1ccccc1N2c1cccc2oc3ccc4c5ccccc5oc4c3c12 ; CC1(C)c2ccccc2N(c2cccc3oc4ccc5c6ccccc6oc5c4c23)c2ccccc21 ; c1ccc2c(c1)oc1c2ccc2oc3cccc(-n4c5ccccc5c5ccccc54)c3c21 ; c1ccc(-c2ccc3c(c2)c2cc(-c4ccccc4)ccc2n3-c2cccc3oc4ccc5c6ccccc6oc5c4c23)cc1]

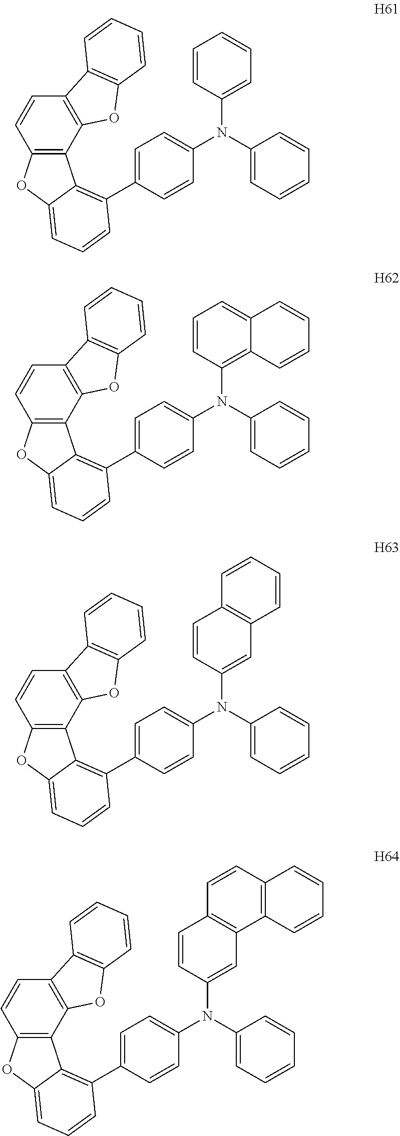

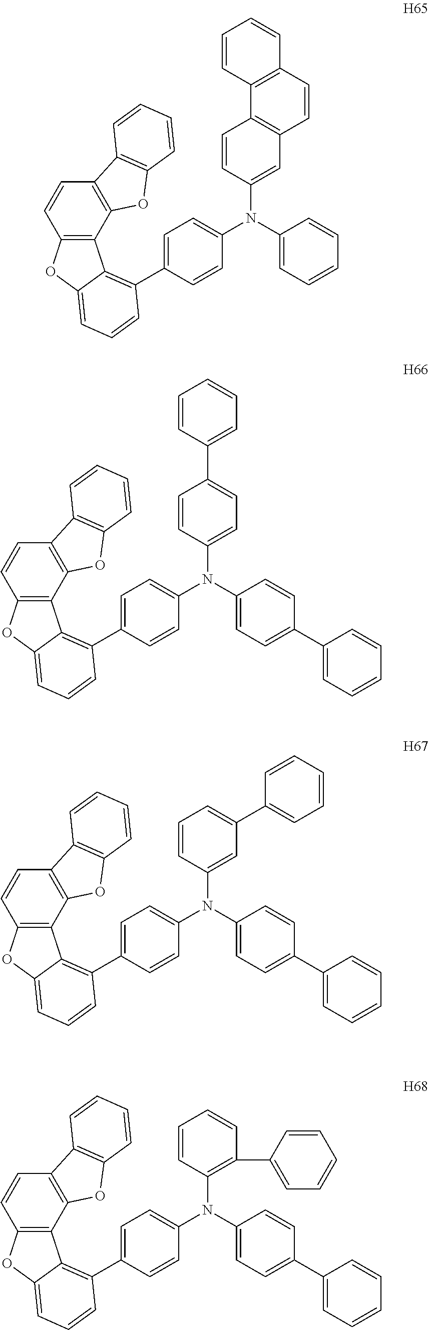 [SMILES: c1ccc(N(c2ccc(-c3cccc4oc5ccc6c7ccccc7oc6c5c34)cc2)c2ccc3c(ccc4ccccc43)c2)cc1 ; c1ccc(-c2ccc(N(c3ccc(-c4ccccc4)cc3)c3ccc(-c4cccc5oc6ccc7c8ccccc8oc7c6c45)cc3)cc2)cc1 ; c1ccc(-c2ccc(N(c3ccc(-c4cccc5oc6ccc7c8ccccc8oc7c6c45)cc3)c3cccc(-c4ccccc4)c3)cc2)cc1 ; c1ccc(-c2ccc(N(c3ccc(-c4cccc5oc6ccc7c8ccccc8oc7c6c45)cc3)c3ccccc3-c3ccccc3)cc2)cc1]

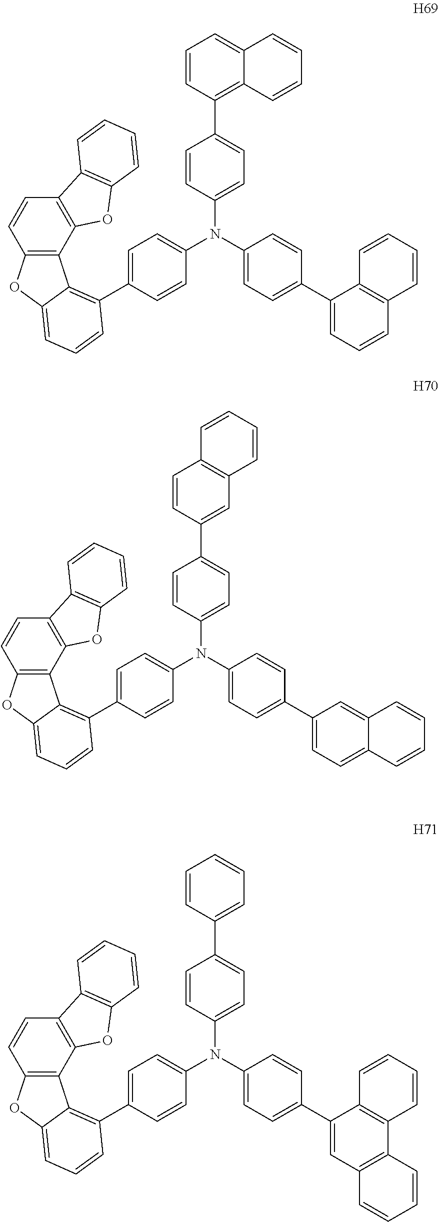 [SMILES: c1ccc2c(-c3ccc(N(c4ccc(-c5cccc6ccccc56)cc4)c4ccc(-c5cccc6oc7ccc8c9ccccc9oc8c7c56)cc4)cc3)cccc2c1 ; c1ccc2cc(-c3ccc(N(c4ccc(-c5ccc6ccccc6c5)cc4)c4ccc(-c5cccc6oc7ccc8c9ccccc9oc8c7c56)cc4)cc3)ccc2c1 ; c1ccc(-c2ccc(N(c3ccc(-c4cc5ccccc5c5ccccc45)cc3)c3ccc(-c4cccc5oc6ccc7c8ccccc8oc7c6c45)cc3)cc2)cc1]

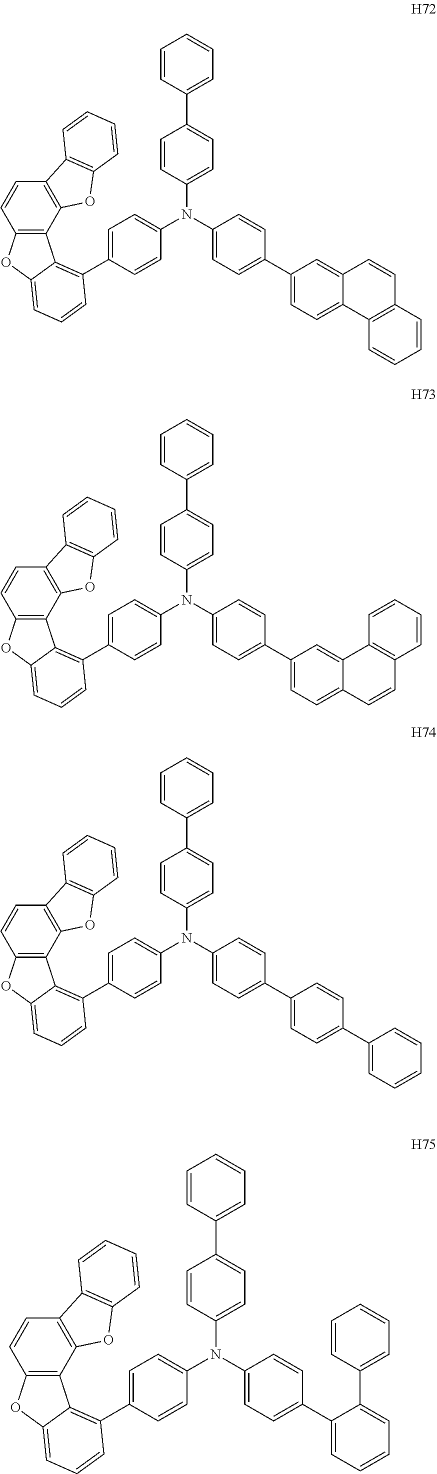 [SMILES: c1ccc(-c2ccc(N(c3ccc(-c4ccc5c(ccc6ccccc65)c4)cc3)c3ccc(-c4cccc5oc6ccc7c8ccccc8oc7c6c45)cc3)cc2)cc1 ; c1ccc(-c2ccc(N(c3ccc(-c4ccc5ccc6ccccc6c5c4)cc3)c3ccc(-c4cccc5oc6ccc7c8ccccc8oc7c6c45)cc3)cc2)cc1 ; c1ccc(-c2ccc(-c3ccc(N(c4ccc(-c5ccccc5)cc4)c4ccc(-c5cccc6oc7ccc8c9ccccc9oc8c7c56)cc4)cc3)cc2)cc1 ; c1ccc(-c2ccc(N(c3ccc(-c4ccccc4-c4ccccc4)cc3)c3ccc(-c4cccc5oc6ccc7c8ccccc8oc7c6c45)cc3)cc2)cc1]

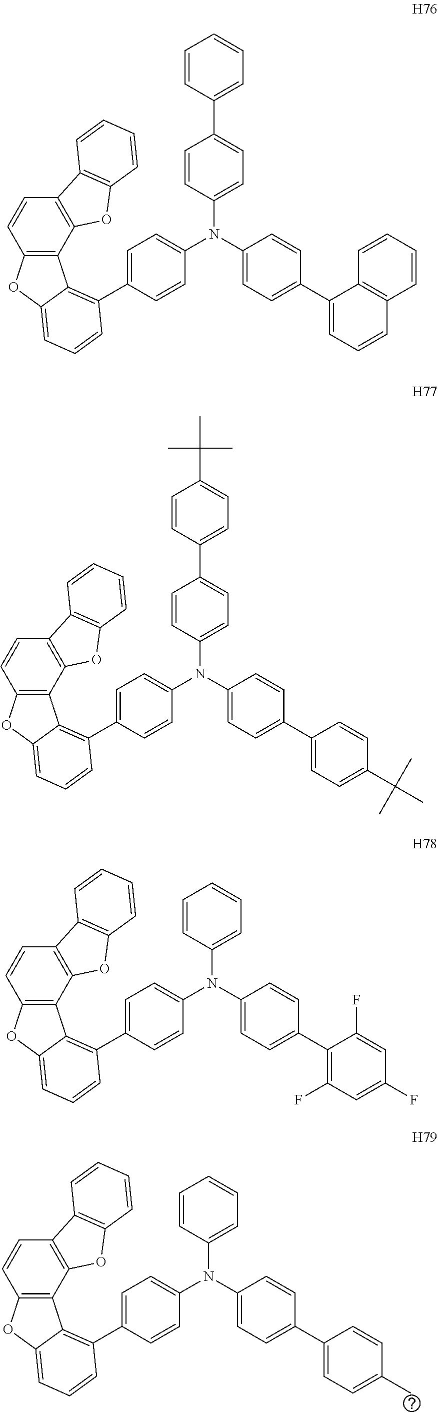 [SMILES: c1ccc(-c2ccc(N(c3ccc(-c4cccc5ccccc45)cc3)c3ccc(-c4cccc5oc6ccc7c8ccccc8oc7c6c45)cc3)cc2)cc1 ; CC(C)(C)c1ccc(-c2ccc(N(c3ccc(-c4ccc(C(C)(C)C)cc4)cc3)c3ccc(-c4cccc5oc6ccc7c8ccccc8oc7c6c45)cc3)cc2)cc1 ; Fc1cc(F)c(-c2ccc(N(c3ccccc3)c3ccc(-c4cccc5oc6ccc7c8ccccc8oc7c6c45)cc3)cc2)c(F)c1 ; Cc1ccc(-c2ccc(N(c3ccccc3)c3ccc(-c4cccc5oc6ccc7c8ccccc8oc7c6c45)cc3)cc2)cc1]

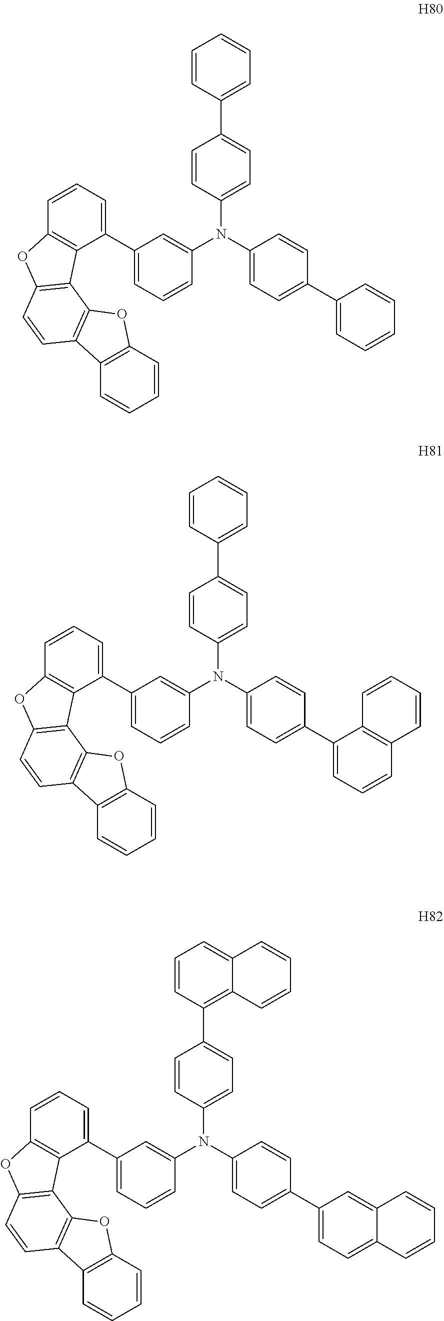 [SMILES: c1ccc(-c2ccc(N(c3ccc(-c4ccccc4)cc3)c3cccc(-c4cccc5oc6ccc7c8ccccc8oc7c6c45)c3)cc2)cc1 ; c1ccc(-c2ccc(N(c3ccc(-c4cccc5ccccc45)cc3)c3cccc(-c4cccc5oc6ccc7c8ccccc8oc7c6c45)c3)cc2)cc1 ; c1cc(-c2cccc3oc4ccc5c6ccccc6oc5c4c23)cc(N(c2ccc(-c3ccc4ccccc4c3)cc2)c2ccc(-c3cccc4ccccc34)cc2)c1]

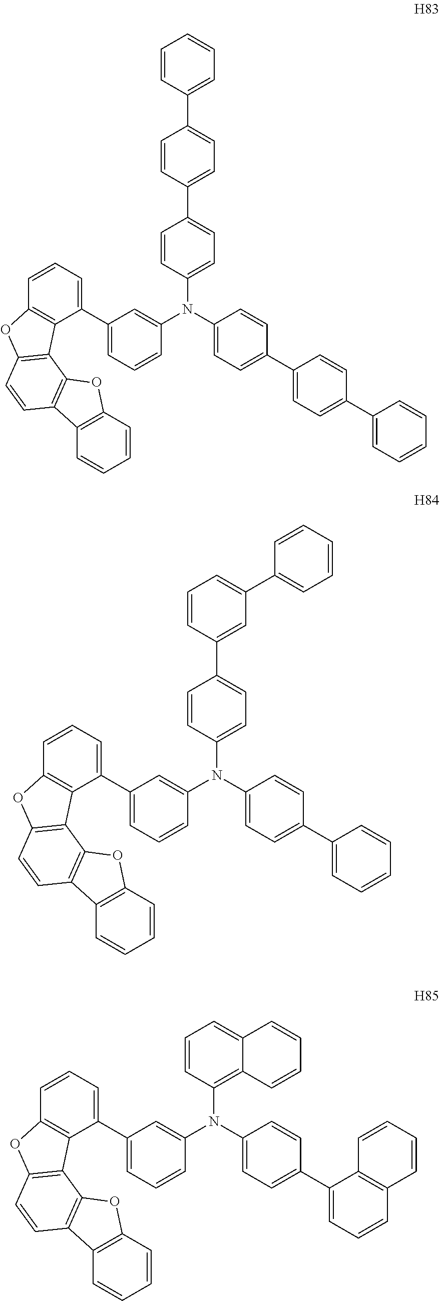 [SMILES: c1ccc(-c2ccc(-c3ccc(N(c4ccc(-c5ccc(-c6ccccc6)cc5)cc4)c4cccc(-c5cccc6oc7ccc8c9ccccc9oc8c7c56)c4)cc3)cc2)cc1 ; c1ccc(-c2ccc(N(c3ccc(-c4cccc(-c5ccccc5)c4)cc3)c3cccc(-c4cccc5oc6ccc7c8ccccc8oc7c6c45)c3)cc2)cc1 ; c1cc(-c2cccc3oc4ccc5c6ccccc6oc5c4c23)cc(N(c2ccc(-c3cccc4ccccc34)cc2)c2cccc3ccccc23)c1]

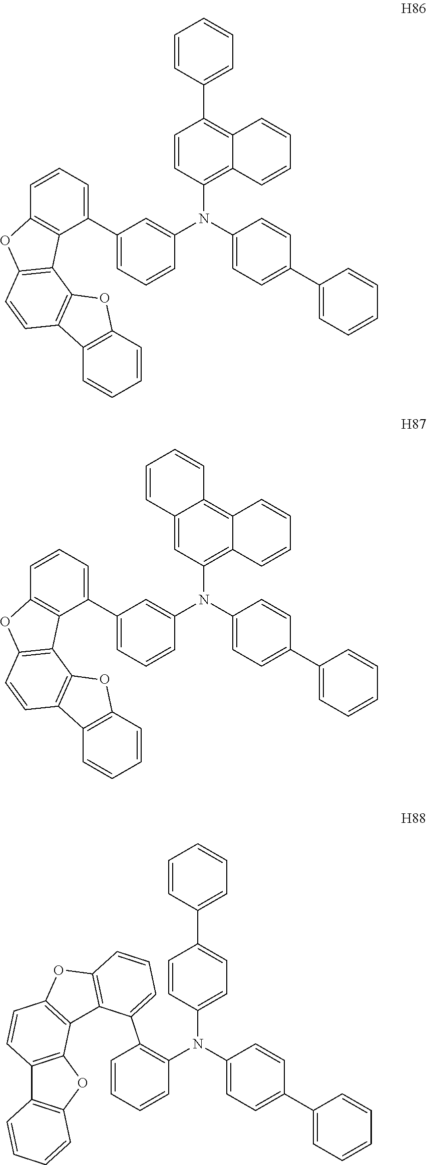 [SMILES: c1ccc(-c2ccc(N(c3cccc(-c4cccc5oc6ccc7c8ccccc8oc7c6c45)c3)c3ccc(-c4ccccc4)c4ccccc34)cc2)cc1 ; c1ccc(-c2ccc(N(c3cccc(-c4cccc5oc6ccc7c8ccccc8oc7c6c45)c3)c3cc4ccccc4c4ccccc34)cc2)cc1 ; c1ccc(-c2ccc(N(c3ccc(-c4ccccc4)cc3)c3ccccc3-c3cccc4oc5ccc6c7ccccc7oc6c5c34)cc2)cc1]

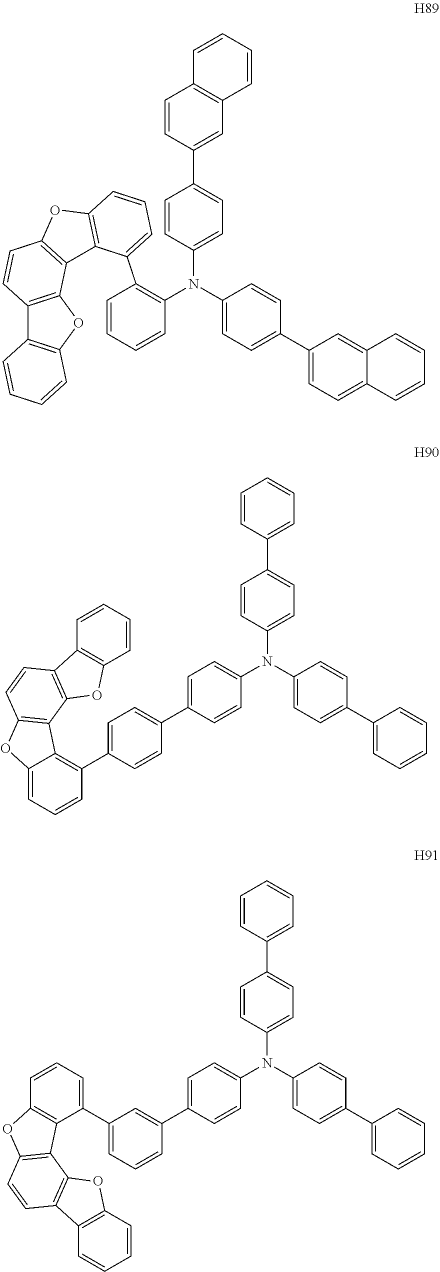 [SMILES: c1ccc(N(c2ccc(-c3ccc4ccccc4c3)cc2)c2ccc(-c3ccc4ccccc4c3)cc2)c(-c2cccc3oc4ccc5c6ccccc6oc5c4c23)c1 ; c1ccc(-c2ccc(N(c3ccc(-c4ccccc4)cc3)c3ccc(-c4ccc(-c5cccc6oc7ccc8c9ccccc9oc8c7c56)cc4)cc3)cc2)cc1 ; c1ccc(-c2ccc(N(c3ccc(-c4ccccc4)cc3)c3ccc(-c4cccc(-c5cccc6oc7ccc8c9ccccc9oc8c7c56)c4)cc3)cc2)cc1]

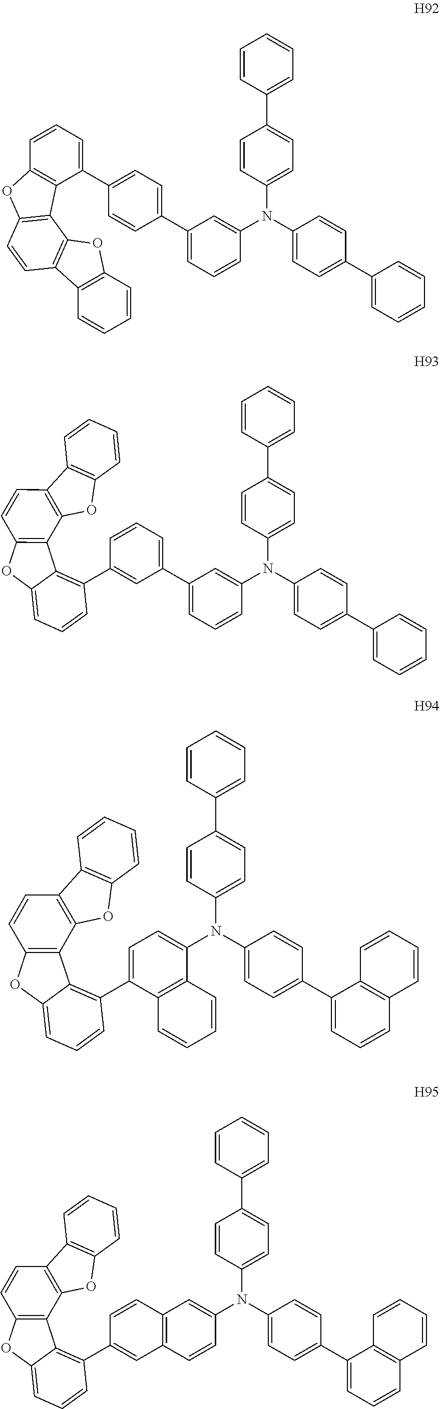 [SMILES: c1ccc(-c2ccc(N(c3ccc(-c4ccccc4)cc3)c3cccc(-c4ccc(-c5cccc6oc7ccc8c9ccccc9oc8c7c56)cc4)c3)cc2)cc1 ; c1ccc(-c2ccc(N(c3ccc(-c4ccccc4)cc3)c3cccc(-c4cccc(-c5cccc6oc7ccc8c9ccccc9oc8c7c56)c4)c3)cc2)cc1 ; c1ccc(-c2ccc(N(c3ccc(-c4cccc5ccccc45)cc3)c3ccc(-c4cccc5oc6ccc7c8ccccc8oc7c6c45)c4ccccc34)cc2)cc1 ; c1ccc(-c2ccc(N(c3ccc(-c4cccc5ccccc45)cc3)c3ccc4cc(-c5cccc6oc7ccc8c9ccccc9oc8c7c56)ccc4c3)cc2)cc1]

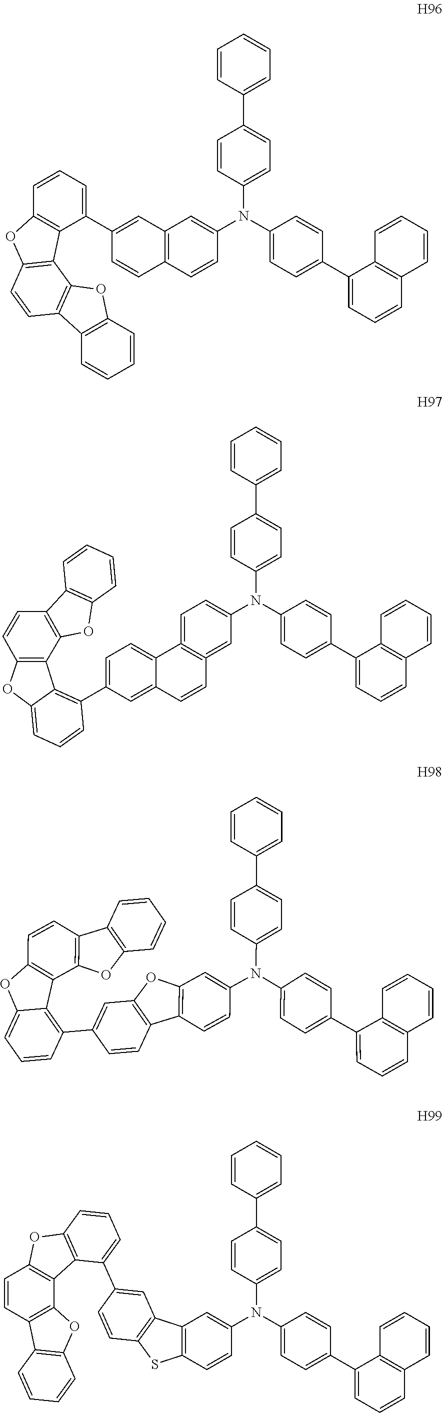

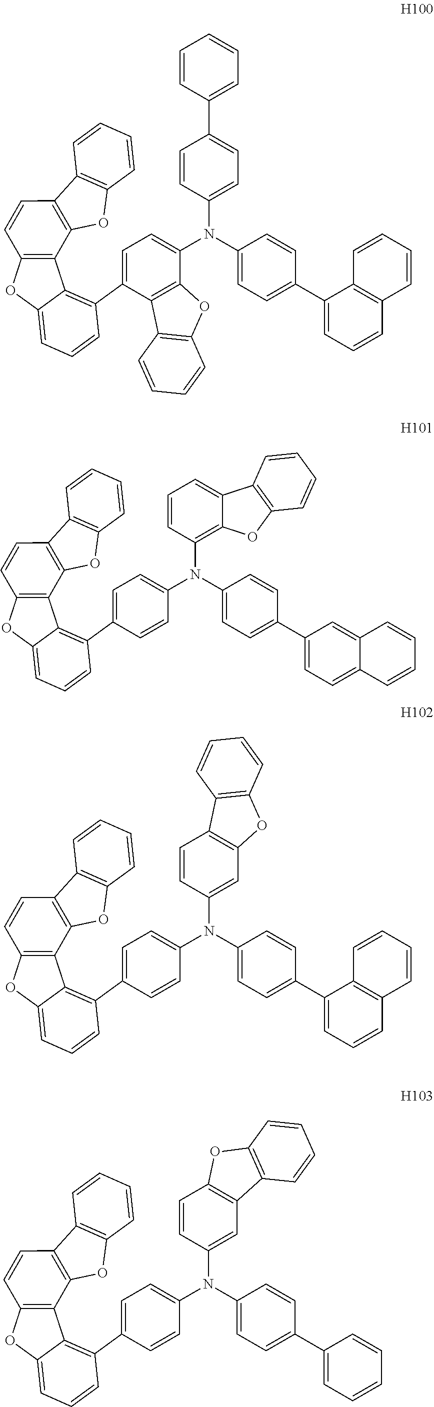 [SMILES: c1ccc(-c2ccc(N(c3ccc(-c4cccc5ccccc45)cc3)c3ccc(-c4cccc5oc6ccc7c8ccccc8oc7c6c45)c4c3oc3ccccc34)cc2)cc1 ; c1ccc2cc(-c3ccc(N(c4ccc(-c5cccc6oc7ccc8c9ccccc9oc8c7c56)cc4)c4cccc5c4oc4ccccc45)cc3)ccc2c1 ; c1ccc2c(-c3ccc(N(c4ccc(-c5cccc6oc7ccc8c9ccccc9oc8c7c56)cc4)c4ccc5c(c4)oc4ccccc45)cc3)cccc2c1 ; c1ccc(-c2ccc(N(c3ccc(-c4cccc5oc6ccc7c8ccccc8oc7c6c45)cc3)c3ccc4oc5ccccc5c4c3)cc2)cc1]

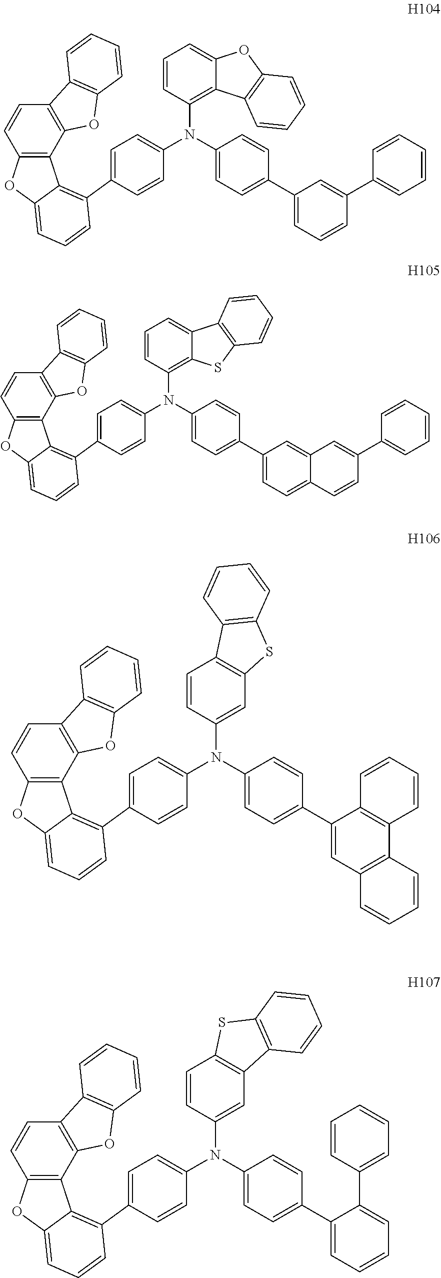 [SMILES: c1ccc(-c2cccc(-c3ccc(N(c4ccc(-c5cccc6oc7ccc8c9ccccc9oc8c7c56)cc4)c4cccc5oc6ccccc6c45)cc3)c2)cc1 ; c1ccc(-c2ccc3ccc(-c4ccc(N(c5ccc(-c6cccc7oc8ccc9c%10ccccc%10oc9c8c67)cc5)c5cccc6c5sc5ccccc56)cc4)cc3c2)cc1 ; c1ccc2c(c1)cc(-c1ccc(N(c3ccc(-c4cccc5oc6ccc7c8ccccc8oc7c6c45)cc3)c3ccc4c(c3)sc3ccccc34)cc1)c1ccccc12 ; c1ccc(-c2ccccc2-c2ccc(N(c3ccc(-c4cccc5oc6ccc7c8ccccc8oc7c6c45)cc3)c3ccc4sc5ccccc5c4c3)cc2)cc1]

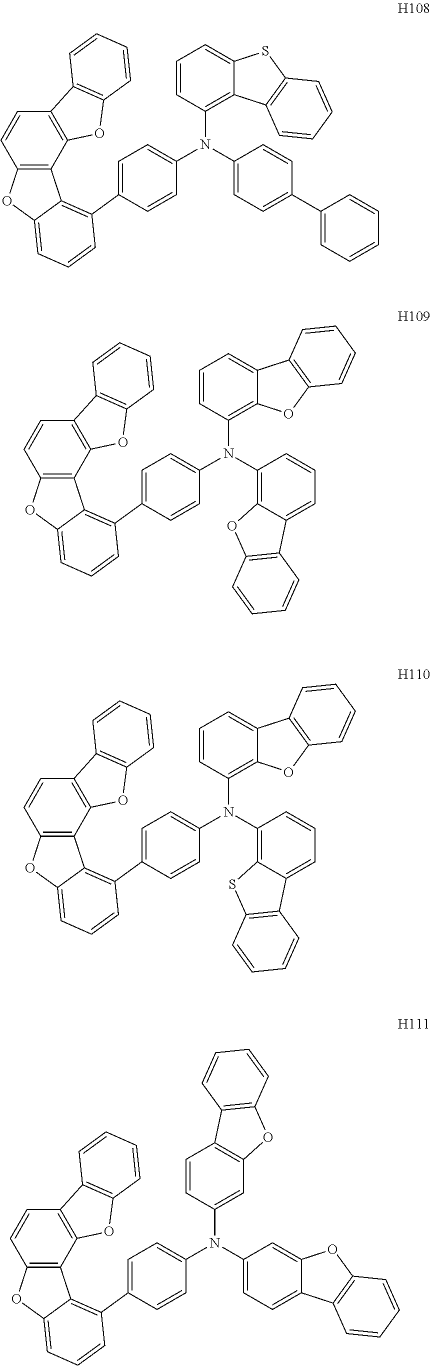 [SMILES: c1ccc(-c2ccc(N(c3ccc(-c4cccc5oc6ccc7c8ccccc8oc7c6c45)cc3)c3cccc4sc5ccccc5c34)cc2)cc1 ; c1ccc2c(c1)oc1c(N(c3ccc(-c4cccc5oc6ccc7c8ccccc8oc7c6c45)cc3)c3cccc4c3oc3ccccc34)cccc12 ; c1ccc2c(c1)oc1c(N(c3ccc(-c4cccc5oc6ccc7c8ccccc8oc7c6c45)cc3)c3cccc4c3sc3ccccc34)cccc12 ; c1ccc2c(c1)oc1cc(N(c3ccc(-c4cccc5oc6ccc7c8ccccc8oc7c6c45)cc3)c3ccc4c(c3)oc3ccccc34)ccc12]

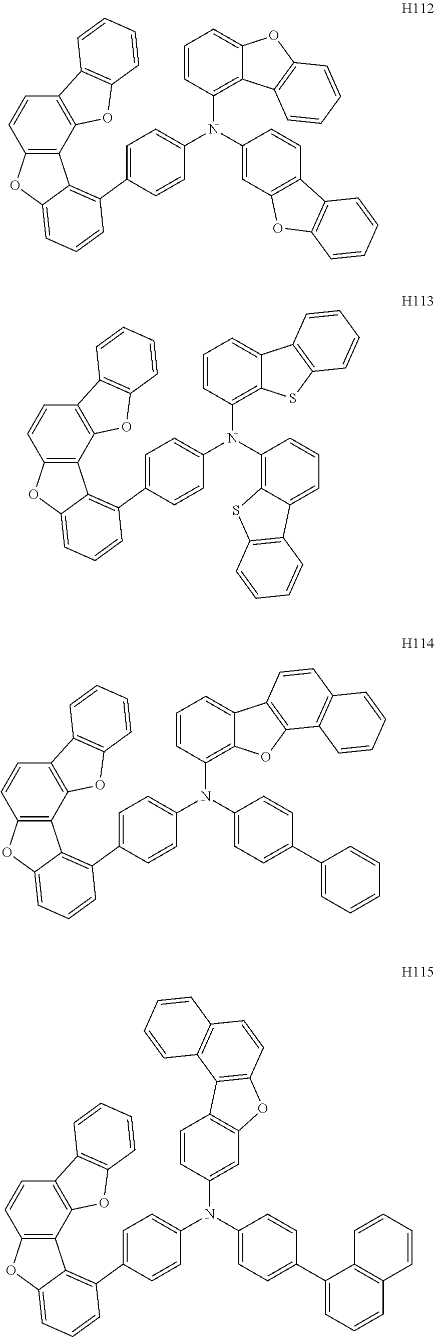 [SMILES: c1ccc2c(c1)oc1cc(N(c3ccc(-c4cccc5oc6ccc7c8ccccc8oc7c6c45)cc3)c3cccc4oc5ccccc5c34)ccc12 ; c1ccc2c(c1)oc1c2ccc2oc3cccc(-c4ccc(N(c5cccc6c5sc5ccccc56)c5cccc6c5sc5ccccc56)cc4)c3c21 ; c1ccc(-c2ccc(N(c3ccc(-c4cccc5oc6ccc7c8ccccc8oc7c6c45)cc3)c3cccc4c3oc3c5ccccc5ccc43)cc2)cc1 ; c1ccc2c(-c3ccc(N(c4ccc(-c5cccc6oc7ccc8c9ccccc9oc8c7c56)cc4)c4ccc5c(c4)oc4ccc6ccccc6c45)cc3)cccc2c1]

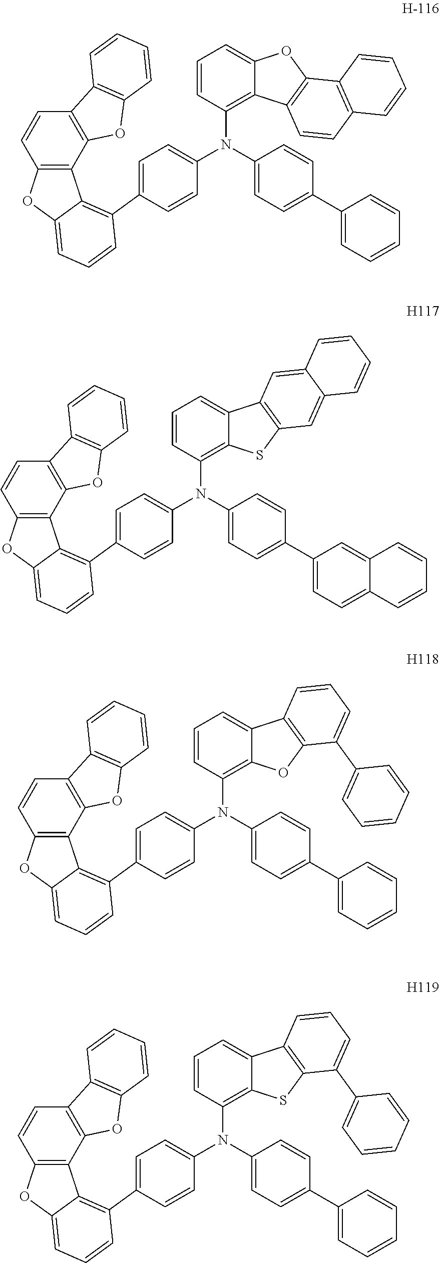 [SMILES: c1ccc(-c2ccc(N(c3ccc(-c4cccc5oc6ccc7c8ccccc8oc7c6c45)cc3)c3cccc4oc5c6ccccc6ccc5c34)cc2)cc1 ; c1ccc2cc(-c3ccc(N(c4ccc(-c5cccc6oc7ccc8c9ccccc9oc8c7c56)cc4)c4cccc5c4sc4cc6ccccc6cc45)cc3)ccc2c1 ; c1ccc(-c2ccc(N(c3ccc(-c4cccc5oc6ccc7c8ccccc8oc7c6c45)cc3)c3cccc4c3oc3c(-c5ccccc5)cccc34)cc2)cc1 ; c1ccc(-c2ccc(N(c3ccc(-c4cccc5oc6ccc7c8ccccc8oc7c6c45)cc3)c3cccc4c3sc3c(-c5ccccc5)cccc34)cc2)cc1]

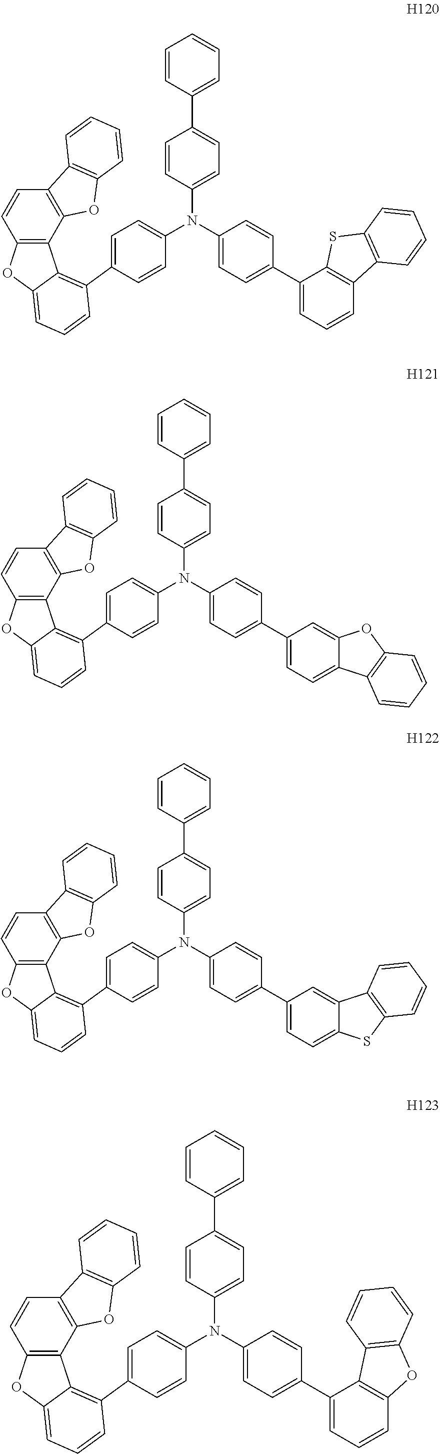 [SMILES: c1ccc(-c2ccc(N(c3ccc(-c4cccc5c4sc4ccccc45)cc3)c3ccc(-c4cccc5oc6ccc7c8ccccc8oc7c6c45)cc3)cc2)cc1 ; c1ccc(-c2ccc(N(c3ccc(-c4ccc5c(c4)oc4ccccc45)cc3)c3ccc(-c4cccc5oc6ccc7c8ccccc8oc7c6c45)cc3)cc2)cc1 ; c1ccc(-c2ccc(N(c3ccc(-c4ccc5sc6ccccc6c5c4)cc3)c3ccc(-c4cccc5oc6ccc7c8ccccc8oc7c6c45)cc3)cc2)cc1 ; c1ccc(-c2ccc(N(c3ccc(-c4cccc5oc6ccccc6c45)cc3)c3ccc(-c4cccc5oc6ccc7c8ccccc8oc7c6c45)cc3)cc2)cc1]

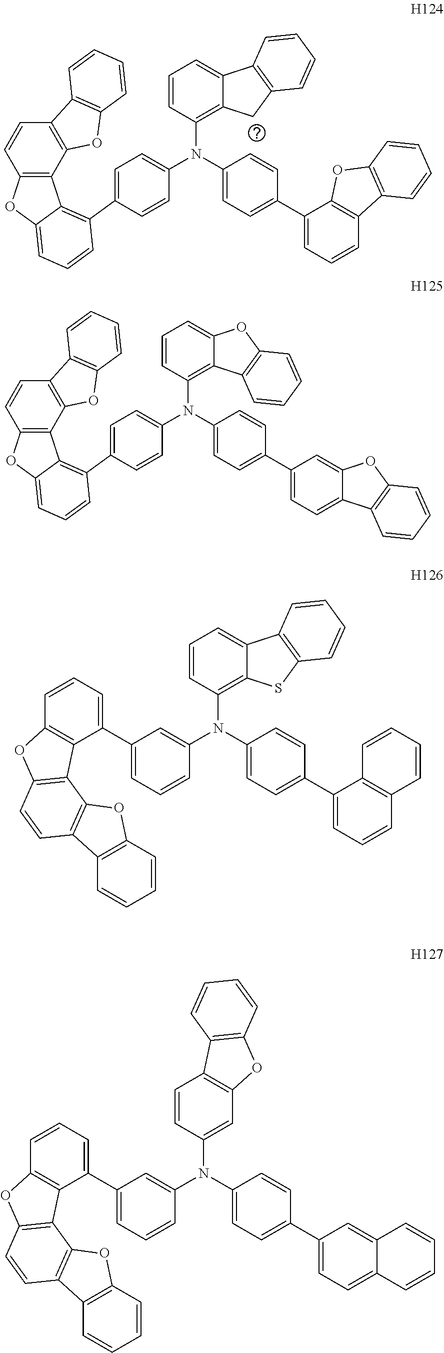 [SMILES: c1ccc2c(c1)Cc1c-2cccc1N(c1ccc(-c2cccc3c2oc2ccccc23)cc1)c1ccc(-c2cccc3oc4ccc5c6ccccc6oc5c4c23)cc1 ; c1ccc2c(c1)oc1cc(-c3ccc(N(c4ccc(-c5cccc6oc7ccc8c9ccccc9oc8c7c56)cc4)c4cccc5oc6ccccc6c45)cc3)ccc12 ; c1cc(-c2cccc3oc4ccc5c6ccccc6oc5c4c23)cc(N(c2ccc(-c3cccc4ccccc34)cc2)c2cccc3c2sc2ccccc23)c1 ; c1cc(-c2cccc3oc4ccc5c6ccccc6oc5c4c23)cc(N(c2ccc(-c3ccc4ccccc4c3)cc2)c2ccc3c(c2)oc2ccccc23)c1]

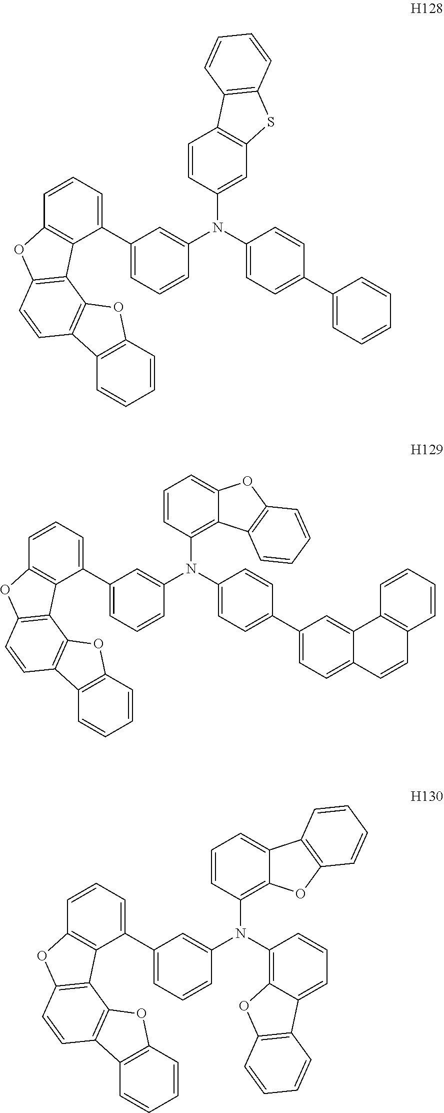 [SMILES: c1ccc(-c2ccc(N(c3cccc(-c4cccc5oc6ccc7c8ccccc8oc7c6c45)c3)c3ccc4c(c3)sc3ccccc34)cc2)cc1 ; c1cc(-c2cccc3oc4ccc5c6ccccc6oc5c4c23)cc(N(c2ccc(-c3ccc4ccc5ccccc5c4c3)cc2)c2cccc3oc4ccccc4c23)c1 ; c1cc(-c2cccc3oc4ccc5c6ccccc6oc5c4c23)cc(N(c2cccc3c2oc2ccccc23)c2cccc3c2oc2ccccc23)c1]

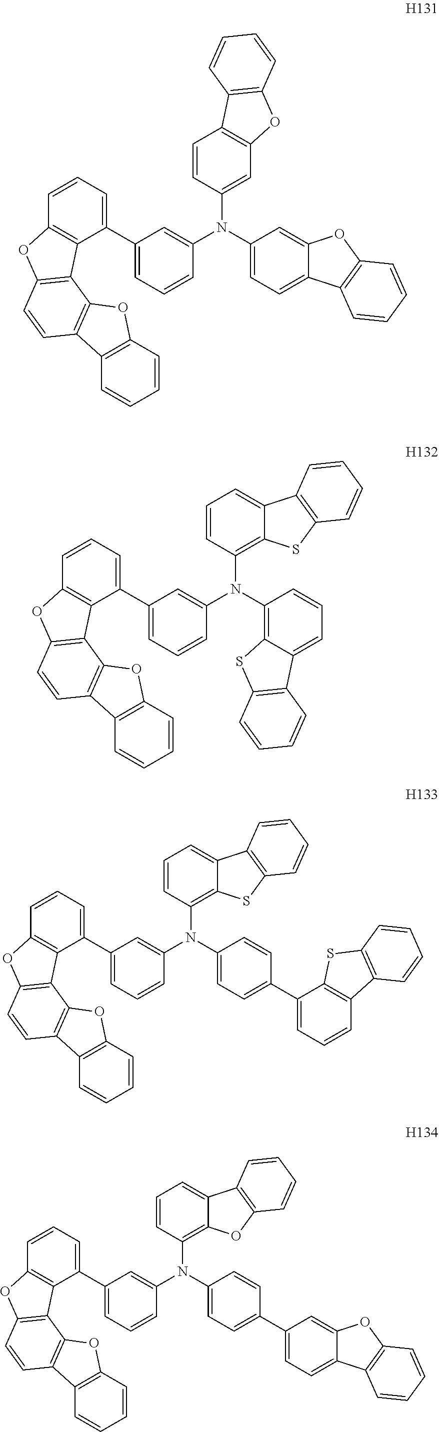 [SMILES: c1cc(-c2cccc3oc4ccc5c6ccccc6oc5c4c23)cc(N(c2ccc3c(c2)oc2ccccc23)c2ccc3c(c2)oc2ccccc23)c1 ; c1cc(-c2cccc3oc4ccc5c6ccccc6oc5c4c23)cc(N(c2cccc3c2sc2ccccc23)c2cccc3c2sc2ccccc23)c1 ; c1cc(-c2cccc3oc4ccc5c6ccccc6oc5c4c23)cc(N(c2ccc(-c3cccc4c3sc3ccccc34)cc2)c2cccc3c2sc2ccccc23)c1 ; c1cc(-c2cccc3oc4ccc5c6ccccc6oc5c4c23)cc(N(c2ccc(-c3ccc4c(c3)oc3ccccc34)cc2)c2cccc3c2oc2ccccc23)c1]

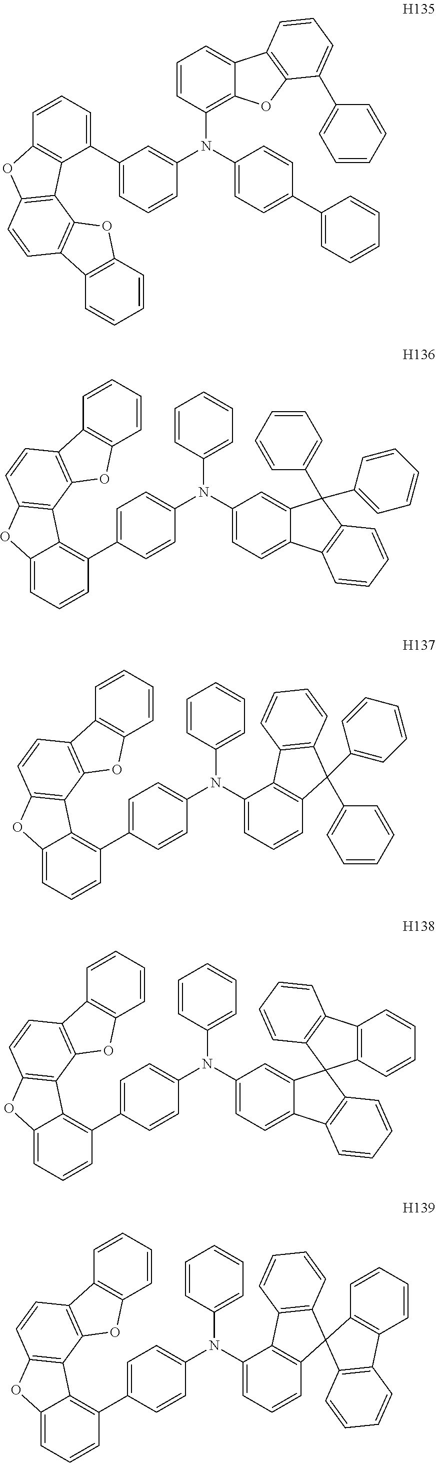 [SMILES: c1ccc(-c2ccc(N(c3cccc(-c4cccc5oc6ccc7c8ccccc8oc7c6c45)c3)c3cccc4c3oc3c(-c5ccccc5)cccc34)cc2)cc1 ; c1ccc(N(c2ccc(-c3cccc4oc5ccc6c7ccccc7oc6c5c34)cc2)c2ccc3c(c2)C(c2ccccc2)(c2ccccc2)c2ccccc2-3)cc1 ; c1ccc(N(c2ccc(-c3cccc4oc5ccc6c7ccccc7oc6c5c34)cc2)c2cccc3c2-c2ccccc2C3(c2ccccc2)c2ccccc2)cc1 ; c1ccc(N(c2ccc(-c3cccc4oc5ccc6c7ccccc7oc6c5c34)cc2)c2ccc3c(c2)C2(c4ccccc4-c4ccccc42)c2ccccc2-3)cc1 ; c1ccc(N(c2ccc(-c3cccc4oc5ccc6c7ccccc7oc6c5c34)cc2)c2cccc3c2-c2ccccc2C32c3ccccc3-c3ccccc32)cc1]

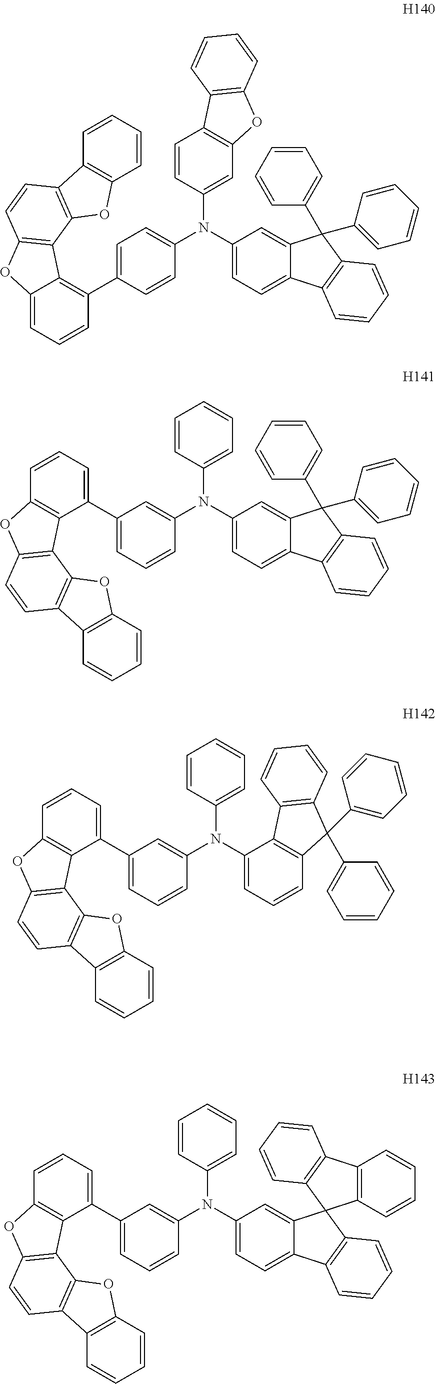 [SMILES: c1ccc(C2(c3ccccc3)c3ccccc3-c3ccc(N(c4ccc(-c5cccc6oc7ccc8c9ccccc9oc8c7c56)cc4)c4ccc5c(c4)oc4ccccc45)cc32)cc1 ; c1ccc(N(c2cccc(-c3cccc4oc5ccc6c7ccccc7oc6c5c34)c2)c2ccc3c(c2)C(c2ccccc2)(c2ccccc2)c2ccccc2-3)cc1 ; c1ccc(N(c2cccc(-c3cccc4oc5ccc6c7ccccc7oc6c5c34)c2)c2cccc3c2-c2ccccc2C3(c2ccccc2)c2ccccc2)cc1 ; c1ccc(N(c2cccc(-c3cccc4oc5ccc6c7ccccc7oc6c5c34)c2)c2ccc3c(c2)C2(c4ccccc4-c4ccccc42)c2ccccc2-3)cc1]

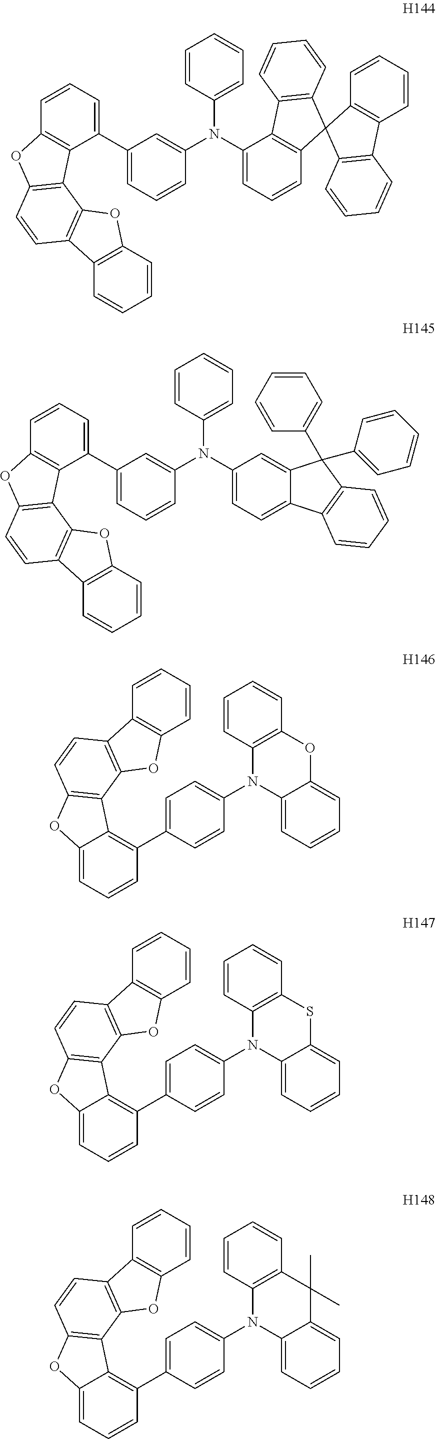 [SMILES: c1ccc(N(c2cccc(-c3cccc4oc5ccc6c7ccccc7oc6c5c34)c2)c2cccc3c2-c2ccccc2C32c3ccccc3-c3ccccc32)cc1 ; c1ccc(N(c2cccc(-c3cccc4oc5ccc6c7ccccc7oc6c5c34)c2)c2ccc3c(c2)C(c2ccccc2)(c2ccccc2)c2ccccc2-3)cc1 ; c1ccc2c(c1)Oc1ccccc1N2c1ccc(-c2cccc3oc4ccc5c6ccccc6oc5c4c23)cc1 ; c1ccc2c(c1)Sc1ccccc1N2c1ccc(-c2cccc3oc4ccc5c6ccccc6oc5c4c23)cc1 ; CC1(C)c2ccccc2N(c2ccc(-c3cccc4oc5ccc6c7ccccc7oc6c5c34)cc2)c2ccccc21]

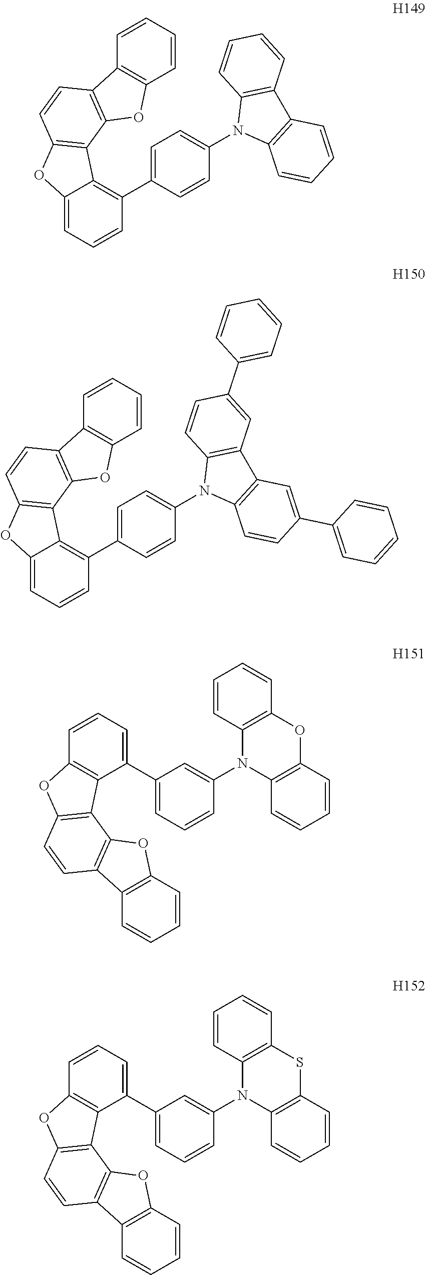 [SMILES: c1ccc2c(c1)oc1c2ccc2oc3cccc(-c4ccc(-n5c6ccccc6c6ccccc65)cc4)c3c21 ; c1ccc(-c2ccc3c(c2)c2cc(-c4ccccc4)ccc2n3-c2ccc(-c3cccc4oc5ccc6c7ccccc7oc6c5c34)cc2)cc1 ; c1cc(-c2cccc3oc4ccc5c6ccccc6oc5c4c23)cc(N2c3ccccc3Oc3ccccc32)c1 ; c1cc(-c2cccc3oc4ccc5c6ccccc6oc5c4c23)cc(N2c3ccccc3Sc3ccccc32)c1]

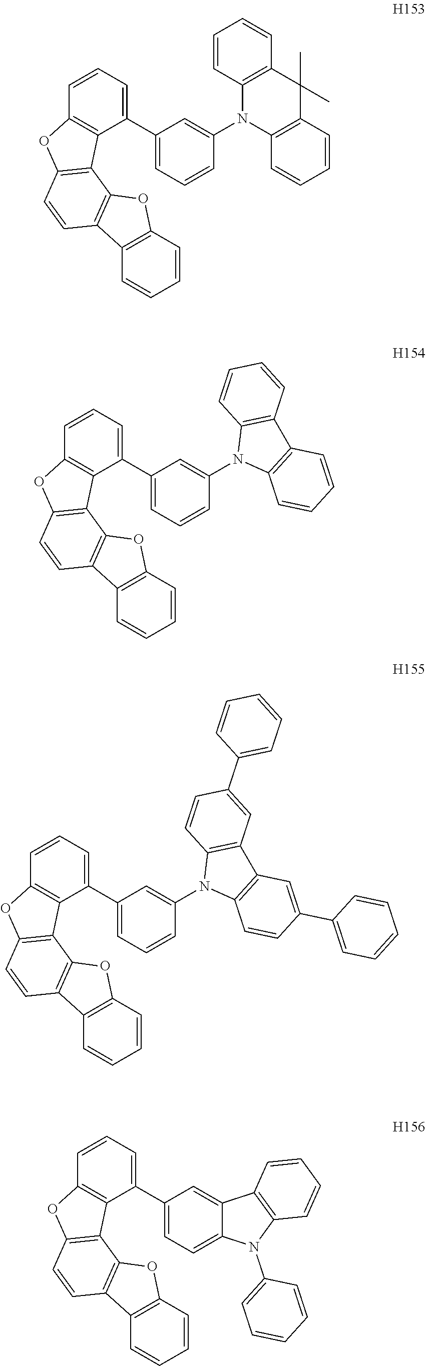 [SMILES: CC1(C)c2ccccc2N(c2cccc(-c3cccc4oc5ccc6c7ccccc7oc6c5c34)c2)c2ccccc21 ; c1cc(-c2cccc3oc4ccc5c6ccccc6oc5c4c23)cc(-n2c3ccccc3c3ccccc32)c1 ; c1ccc(-c2ccc3c(c2)c2cc(-c4ccccc4)ccc2n3-c2cccc(-c3cccc4oc5ccc6c7ccccc7oc6c5c34)c2)cc1 ; c1ccc(-n2c3ccccc3c3cc(-c4cccc5oc6ccc7c8ccccc8oc7c6c45)ccc32)cc1]

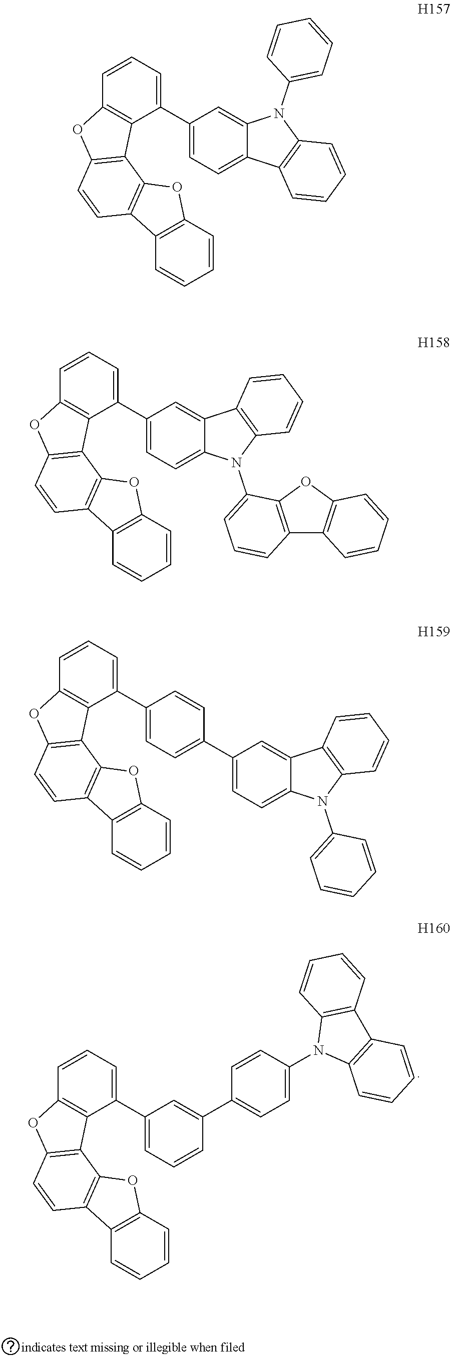 [SMILES: c1ccc(-n2c3ccccc3c3ccc(-c4cccc5oc6ccc7c8ccccc8oc7c6c45)cc32)cc1 ; c1ccc2c(c1)oc1c(-n3c4ccccc4c4cc(-c5cccc6oc7ccc8c9ccccc9oc8c7c56)ccc43)cccc12 ; c1ccc(-n2c3ccccc3c3cc(-c4ccc(-c5cccc6oc7ccc8c9ccccc9oc8c7c56)cc4)ccc32)cc1 ; c1cc(-c2ccc(-n3c4ccccc4c4ccccc43)cc2)cc(-c2cccc3oc4ccc5c6ccccc6oc5c4c23)c1]

21. A polycyclic compound represented by Formula A: 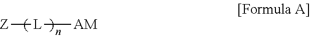
wherein in Formula A,
n is an integer from 0 to 3,
L is a substituted or unsubstituted arylene group having 6 to 40 ring-forming carbon atoms and excluding fluorene, or a substituted or unsubstituted heteroarylene group having 2 to 40 ring-forming carbon atoms and excluding N as a ring-forming atom, and
AM is a substituted or unsubstituted amine group, or a substituted or unsubstituted heterocyclic group including N as a ring-forming atom, and
Z is a group represented by Formula 2-1 or Formula 2-2: 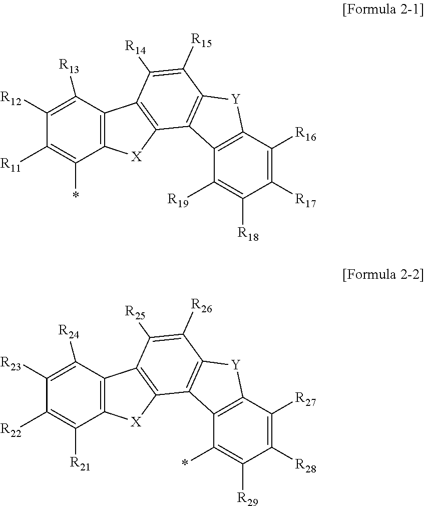
wherein in Formula 2-1 and Formula 2-2,
X and Y are each independently O or S,
R₁₁ to R₁₉ and R₂₁ to R₂₉ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, a substituted or unsubstituted aryl group having 6 to 40 ring-forming carbon atoms, or a substituted or unsubstituted heteroaryl group having 2 to 40 ring-forming carbon atoms and excluding a carbazole group, or are bonded to an adjacent group to form a ring, and
* indicates a binding site to a neighboring atom.

22. The polycyclic compound of claim 21, wherein the heterocyclic group is a substituted or unsubstituted carbazole group, a substituted or unsubstituted phenoxazine group, a substituted or unsubstituted phenothiazine group, or substituted or unsubstituted acridine group.

23. The polycyclic compound of claim 22, wherein AM is a group represented by one of Formulae A1 to A3: 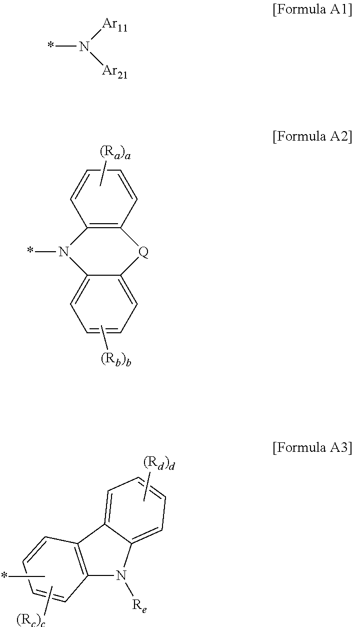
wherein in Formula A1,
Ar₁₁ and Ar₂₁ are each independently a substituted or unsubstituted aryl group having 6 to 40 ring-forming carbon atoms and excluding triphenylene, or a substituted or unsubstituted heteroaryl group having 2 to 40 ring-forming carbon atoms and excluding N as a ring-forming atom,
in Formula A2,
Q is a single bond, O, S, or C(R₁)(R₂), and
R₁ and R₂ are each independently a substituted or unsubstituted alkyl group having 1 to 20 carbon atoms, or a substituted or unsubstituted aryl group having 6 to 40 ring-forming carbon atoms, and
in Formulae A2 and A3,
a, b, and d are each independently an integer from 0 to 4, and
c is an integer from 0 to 3, and
Rₐ, R_{b}, R_{c}, R_{d}, and Rₑ are each independently a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms, or are bonded to an adjacent group to form an aromatic ring, and
in Formulae A1 to A3,
* indicates a binding site to a neighboring atom.

24. The polycyclic compound of claim 21, wherein in Formula A,
L is a direct linkage, a substituted or unsubstituted phenylene group, a substituted or unsubstituted divalent biphenyl group, a substituted or unsubstituted naphthalene group, a substituted or unsubstituted phenanthrene group, a substituted or unsubstituted dibenzofuranylene group, or a substituted or unsubstituted dibenzothiophenylene group.
